(19) United States
(12) Patent Application Publication
(10) Pub. No.: US 2025/0031569 A1
(43) Pub. Date: Jan. 23, 2025

(54) ORGANIC ELECTROLUMINESCENT HOST MATERIAL COMPOSITION, LIGHT EMITTING DEVICE AND APPLICATION

(71) Applicant: NINGBO LUMILAN ADVANCED MATERIALS CO., LTD., Ningbo (CN)

(72) Inventors: Ting-Wei WEI, Ningbo (CN); Fengjiao LIU, Ningbo (CN); Qinghua ZOU, Ningbo (CN); Cong TANG, Ningbo (CN); Xiaoxin RAN, Ningbo (CN); Binyu XU, Ningbo (CN); Xiaoyun XU, Ningbo (CN); Renzhou WANG, Ningbo (CN); Qixiang SONG, Ningbo (CN); Huanda DING, Ningbo (CN); Zhi-Kuan CHEN, Ningbo (CN)

(73) Assignee: NINGBO LUMILAN ADVANCED MATERIALS CO., LTD., Ningbo (CN)

(21) Appl. No.: 18/418,057

(22) Filed: Jan. 19, 2024

(30) Foreign Application Priority Data
Jan. 20, 2023 (CN) 202310110966.X
Jun. 30, 2023 (CN) 202310800187.2

(51) Int. Cl. H10K 85/60

(52) U.S. Cl. CPC H10K 85/654; H10K 85/622; H10K 85/633; H10K 85/6572; H10K 85/6574; H10K 85/6576

(57) ABSTRACT

The present disclosure discloses an organic electroluminescent compound, a composition, a light emitting device and an application. The organic electroluminescent compound is a compound M having a structure shown in Formula (2), and Formula (2) is:

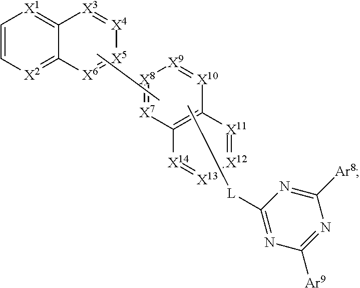

in Formula (2), X¹-X¹⁴ are selected from N or CR⁸, and R⁸ is selected from hydrogen or deuterium; L is selected from a connecting bond, substituted or unsubstituted C6-C30 arylenyl, and substituted or unsubstituted C3-C30 heteroarylenyl; Ar⁸-Ar⁹ are each independently selected from substituted or unsubstituted C6-C30 aryl and substituted or unsubstituted C3-C30 heteroaryl; and substituents are selected from one of or a combination of two of deuterium, halogen, cyano, C1-C6 alkyl, C3-C30 cycloalkyl, C6-C30 aryl and C3-C30 heteroaryl. When the compound M in the present disclosure and a compound N in the present disclosure match with each other to be used as an organic electroluminescent host material of an organic light emitting device, a lighting voltage of the organic light emitting device can be lowered obviously, the light emitting efficiency is improved remarkably, and the service life is prolonged remarkably.

### TECHNICAL FIELD

The present disclosure relates to the organic electroluminescent field, in particular to an organic electroluminescent host material composition, a light emitting device and an application.

### BACKGROUND

An organic light emitting device (OLED) converts electrical energy into light by applying electricity to an organic electroluminescent material, and typically includes an anode, a cathode, and an organic layer formed between these two electrodes. The organic layer of the organic electroluminescent device may contain a hole injection layer, a hole transport layer, a hole auxiliary layer, a light emitting auxiliary layer, an electron blocking layer, a light emitting layer (containing a host material and a dopant material), an electron buffer layer, a hole blocking layer, an electron transport layer, an electron injection layer, etc. Based on the functions achieved by each layer, various materials used in the organic layer are divided into hole injection materials, hole transport materials, hole auxiliary materials, light emitting auxiliary materials, electron blocking materials, light emitting materials, electron buffer materials, hole blocking materials, electron transport materials, electron injection materials, etc. In the organic electroluminescent device, holes from the anode and electrons from the cathode are injected into the light emitting layer by applying voltages, and high-energy excitons are produced by the recombination of the holes and the electrons. An organic light emitting compound emits light from energy moving to an excited state and energy at the time when the organic light emitting compound returns to a ground state from the excited state.

Organic light emitting compounds disclosed currently include amine derivatives of indenotriphenylene, triazine electron transport materials, etc., such as an indenotriphenylene-based amine derivative disclosed in the US patent application US20200115369A1, and a triazine electron transport material, and a preparation method and application thereof disclosed in the Chinese patent application CN113004295A. The currently disclosed indenotriphenylene-based amine derivative can serve as a charge transport layer and an electron blocking layer in an organic electroluminescent device.

However, functional materials composed of existing organic light emitting compounds have low stability and imbalanced carrier mobility, as a result, the problems of high driving voltage, low light emitting efficiency, and short service life of organic electroluminescent diodes are caused, which severely limits applications of the organic electroluminescent diodes.

### SUMMARY OF THE INVENTION

Therefore, the technical problem to be solved by the present disclosure is to solve the problem of high driving voltage of light emitting equipment caused by low stability and imbalanced carrier mobility of organic electroluminescent materials in the prior art, thereby providing an organic electroluminescent host material composition, a light emitting device and an application to solve the above problem.

An organic electroluminescent compound is a compound M having a structure shown in Formula (2),

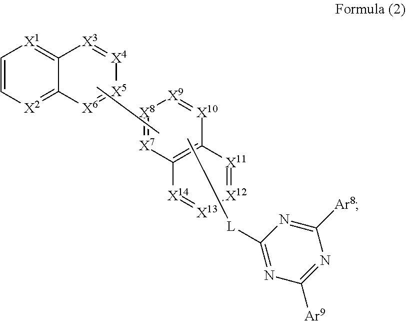

in Formula (2), X¹-X¹⁴ are each independently selected from N or CR⁸, and R⁸ is selected from hydrogen or deuterium;
L is selected from a connecting bond, substituted or unsubstituted C6-C30 arylenyl, and substituted or unsubstituted C3-C30 heteroarylenyl;
Ar⁸-Ar⁹ are each independently selected from substituted or unsubstituted C6-C30 aryl and substituted or unsubstituted C3-C30 heteroaryl;
substituents in the substituted C6-C30 arylenyl, the substituted C3-C30 heteroarylenyl, the substituted C6-C30 aryl and the substituted C3-C30 heteroaryl are selected from one of or a combination of two of deuterium, halogen, cyano, C1-C6 alkyl, C3-C30 cycloalkyl, C6-C30 aryl, and C3-C30 heteroaryl; and
when X¹-X¹⁴ are all CR⁸, Ar⁸-Ar⁹ are not substituted or unsubstituted naphthyl at the same time.

In Formula (2), any one of X¹-X¹⁴ is selected from N, and the remaining is CR⁸; or any one of X¹-X⁶ is selected from N, the remaining is CR⁸, any one of X⁷-X¹⁴ is selected from N, and the remaining is CR⁸; or, X¹-X¹⁴ are all selected from CR⁸; and when a plurality of R⁸ are present, R⁸ is each present independently, and the plurality of R⁸ can be the same or different.

Ar⁸-Ar⁹ are each independently selected from hydrogen, deuterium and the following substituted or unsubstituted groups: phenyl, naphthyl, biphenyl, triphenyl, phenanthryl, fluoranthenyl, triphenylenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, benzothienyl, dibenzothienyl, carbazolyl, benzocarbazolyl, and dibenzocarbazolyl.

Ar⁸-Ar⁹ are each independently selected from hydrogen, deuterium and the following substituted or unsubstituted groups: phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, naphthobenzofuryl, benzothienyl, dibenzothienyl, naphthobenzothienyl, carbazolyl, benzocarbazolyl, benzocarbazolyl, and dibenzocarbazolyl.

The structure of the compound M is as shown in any one of Formula 2-1 to Formula 2-5:

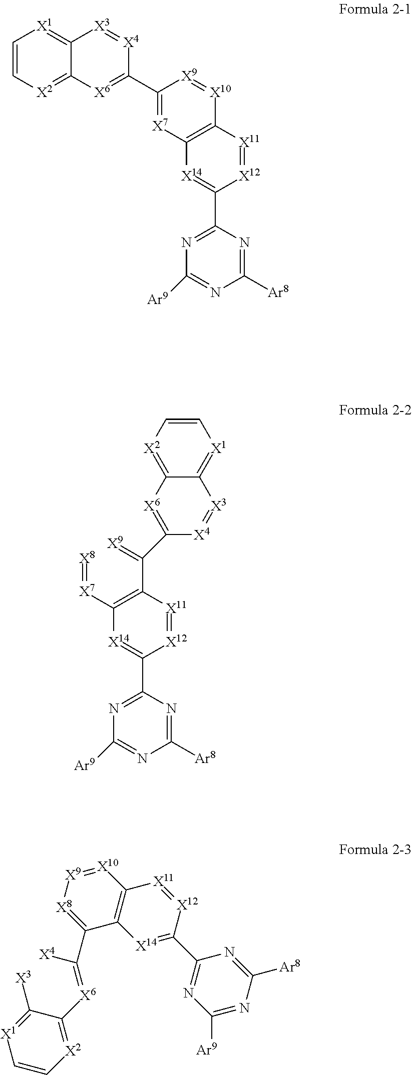

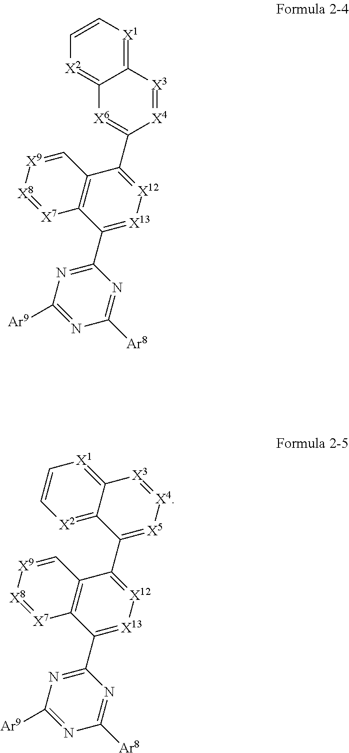

X¹-X¹⁴, Ar⁸-Ar⁹ and L are defined the same as the above.

The structure of the compound M is as shown in any one of Formula 2-6 to Formula 2-28:

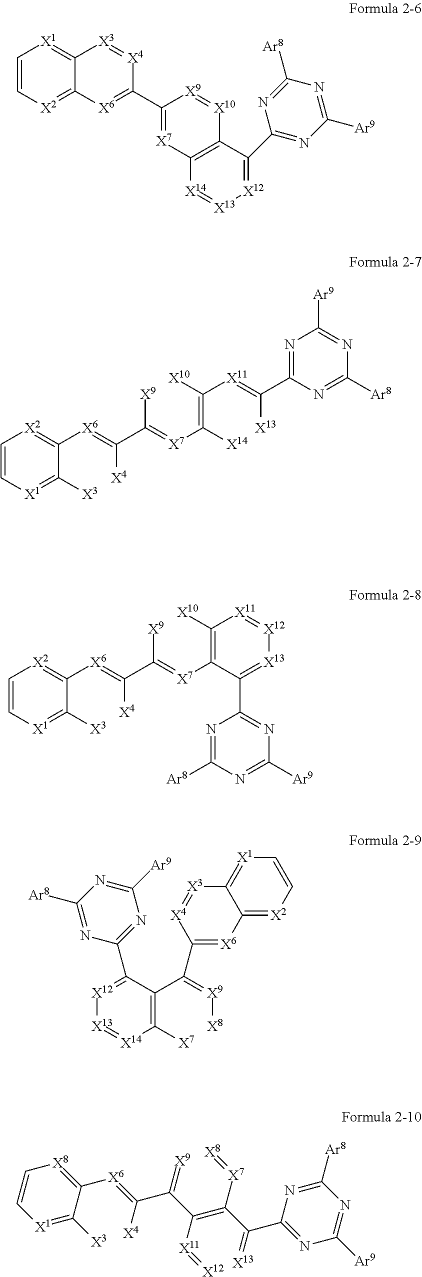

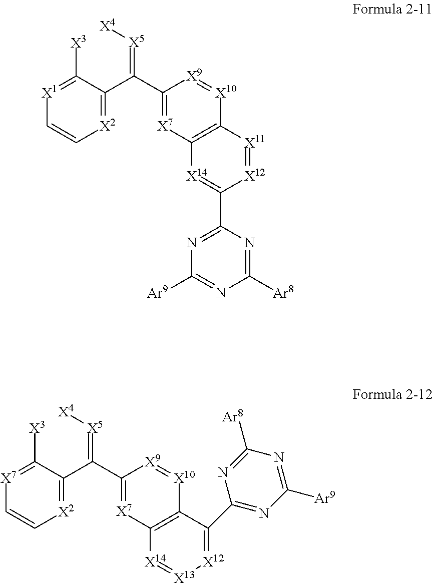

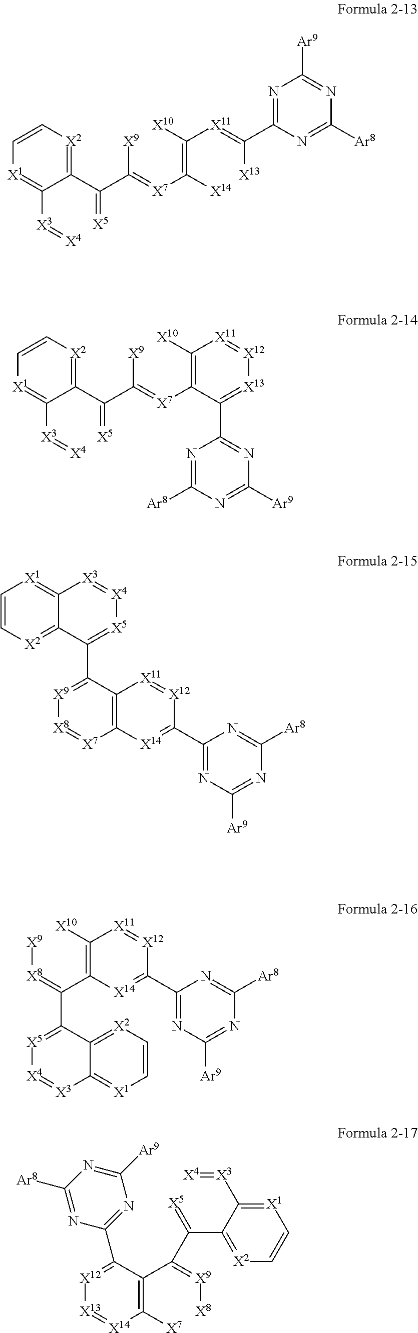

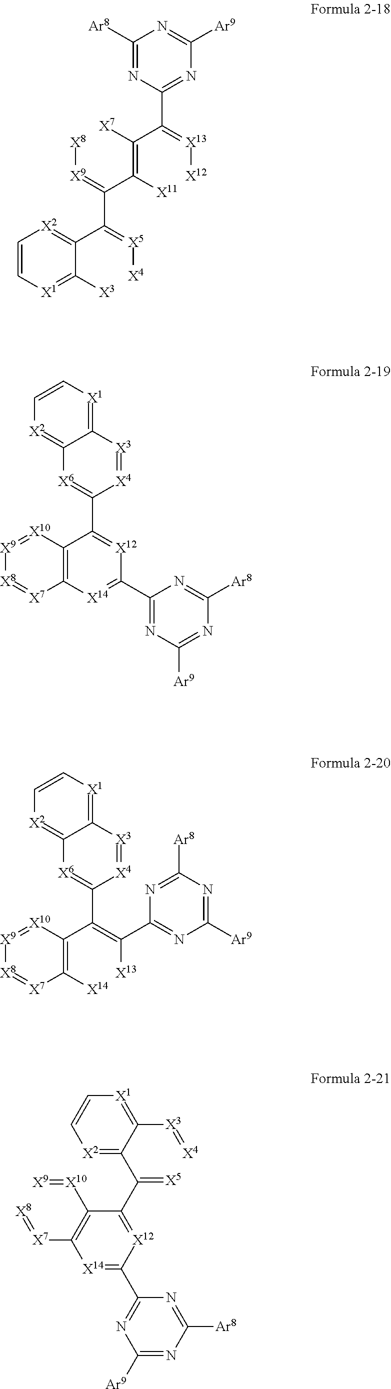

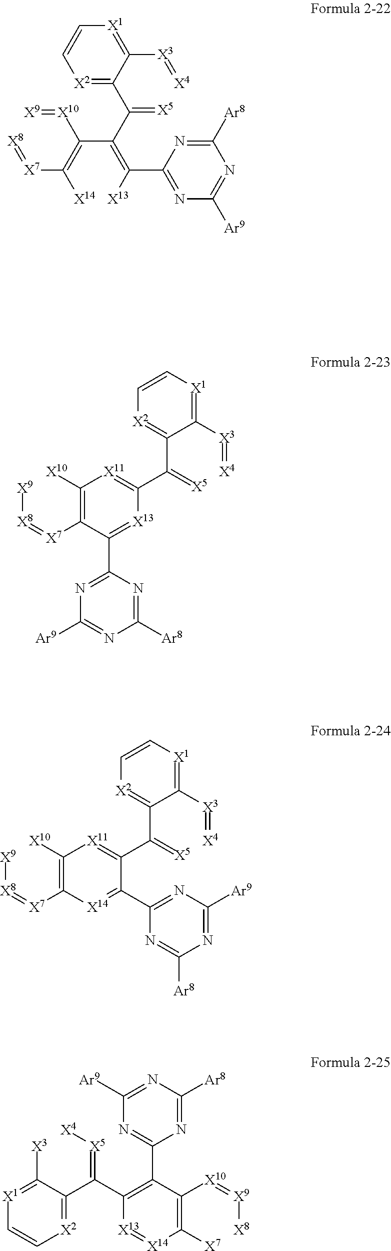

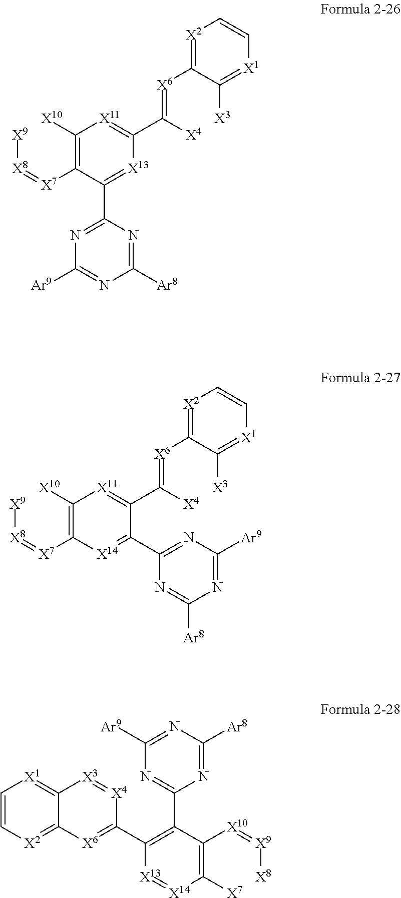

X¹-X¹⁴, Ar⁸-Ar⁹ and L are defined the same as the above.

The structure of the compound M is as shown in any one of M-1 to M-388 below.

The structures of M-1 to M-388 are as follows:

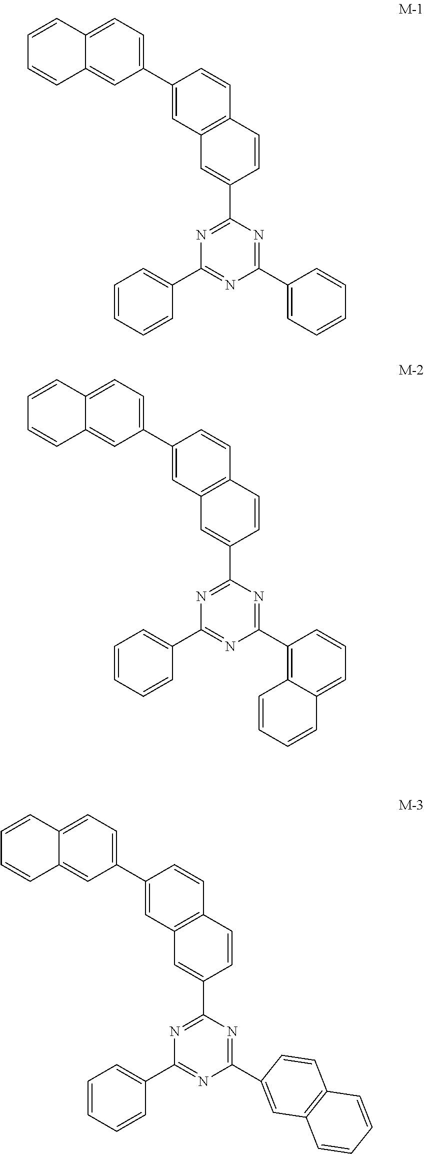 [SMILES: c1ccc(-c2nc(-c3ccccc3)nc(-c3ccc4ccc(-c5ccc6ccccc6c5)cc4c3)n2)cc1 ; c1ccc(-c2nc(-c3ccc4ccc(-c5ccc6ccccc6c5)cc4c3)nc(-c3cccc4ccccc34)n2)cc1 ; c1ccc(-c2nc(-c3ccc4ccccc4c3)nc(-c3ccc4ccc(-c5ccc6ccccc6c5)cc4c3)n2)cc1]

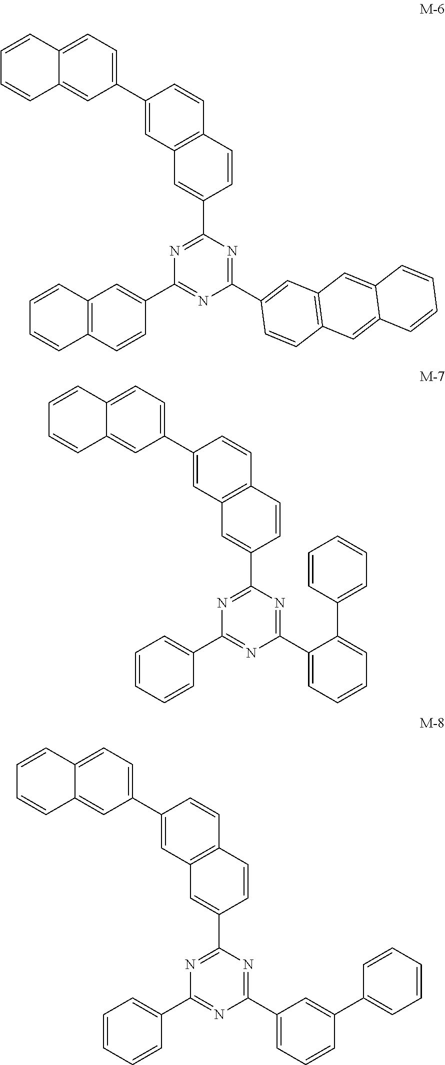

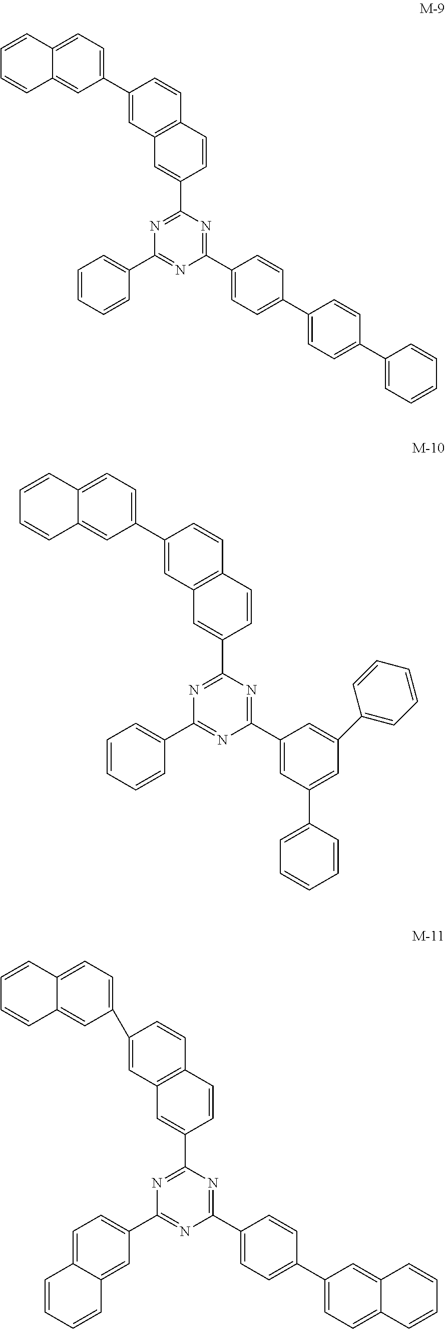 [SMILES: c1ccc(-c2ccc(-c3ccc(-c4nc(-c5ccccc5)nc(-c5ccc6ccc(-c7ccc8ccccc8c7)cc6c5)n4)cc3)cc2)cc1 ; c1ccc(-c2cc(-c3ccccc3)cc(-c3nc(-c4ccccc4)nc(-c4ccc5ccc(-c6ccc7ccccc7c6)cc5c4)n3)c2)cc1 ; c1ccc2cc(-c3ccc(-c4nc(-c5ccc6ccccc6c5)nc(-c5ccc6ccc(-c7ccc8ccccc8c7)cc6c5)n4)cc3)ccc2c1]

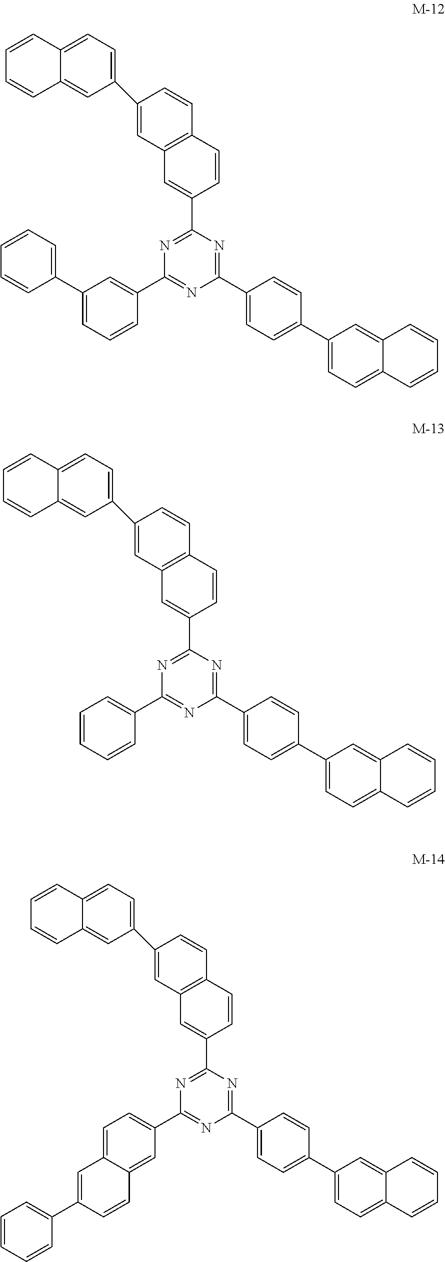 [SMILES: c1ccc(-c2cccc(-c3nc(-c4ccc(-c5ccc6ccccc6c5)cc4)nc(-c4ccc5ccc(-c6ccc7ccccc7c6)cc5c4)n3)c2)cc1 ; c1ccc(-c2nc(-c3ccc(-c4ccc5ccccc5c4)cc3)nc(-c3ccc4ccc(-c5ccc6ccccc6c5)cc4c3)n2)cc1 ; c1ccc(-c2ccc3cc(-c4nc(-c5ccc(-c6ccc7ccccc7c6)cc5)nc(-c5ccc6ccc(-c7ccc8ccccc8c7)cc6c5)n4)ccc3c2)cc1]

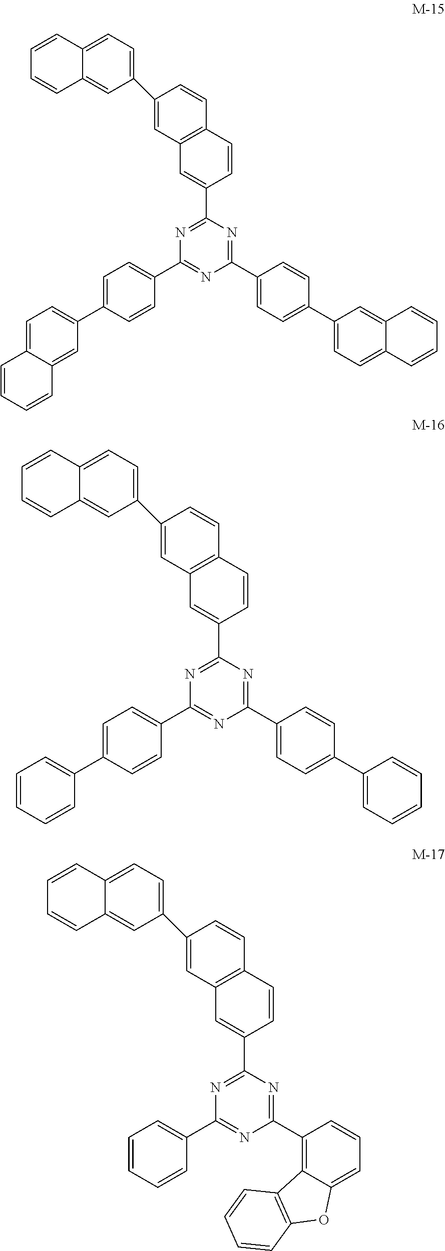 [SMILES: c1ccc2cc(-c3ccc(-c4nc(-c5ccc(-c6ccc7ccccc7c6)cc5)nc(-c5ccc6ccc(-c7ccc8ccccc8c7)cc6c5)n4)cc3)ccc2c1 ; c1ccc(-c2ccc(-c3nc(-c4ccc(-c5ccccc5)cc4)nc(-c4ccc5ccc(-c6ccc7ccccc7c6)cc5c4)n3)cc2)cc1 ; c1ccc(-c2nc(-c3ccc4ccc(-c5ccc6ccccc6c5)cc4c3)nc(-c3cccc4oc5ccccc5c34)n2)cc1]

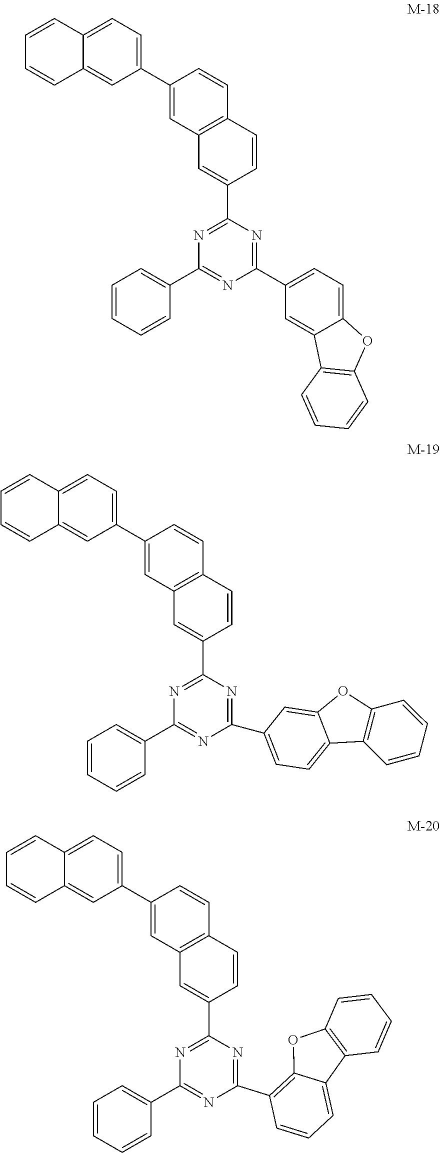 [SMILES: c1ccc(-c2nc(-c3ccc4ccc(-c5ccc6ccccc6c5)cc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4c(c3)oc3ccccc34)n2)cc1 ; c1ccc(-c2nc(-c3ccc4ccc(-c5ccc6ccccc6c5)cc4c3)nc(-c3cccc4c3oc3ccccc34)n2)cc1]

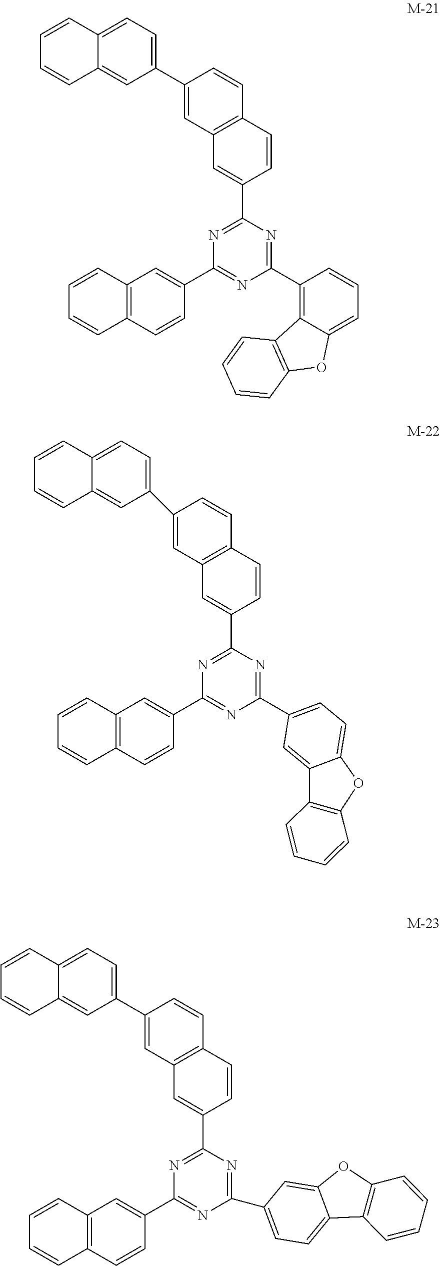 [SMILES: c1ccc2cc(-c3ccc4ccc(-c5nc(-c6ccc7ccccc7c6)nc(-c6cccc7oc8ccccc8c67)n5)cc4c3)ccc2c1 ; c1ccc2cc(-c3ccc4ccc(-c5nc(-c6ccc7ccccc7c6)nc(-c6ccc7oc8ccccc8c7c6)n5)cc4c3)ccc2c1 ; c1ccc2cc(-c3ccc4ccc(-c5nc(-c6ccc7ccccc7c6)nc(-c6ccc7c(c6)oc6ccccc67)n5)cc4c3)ccc2c1]

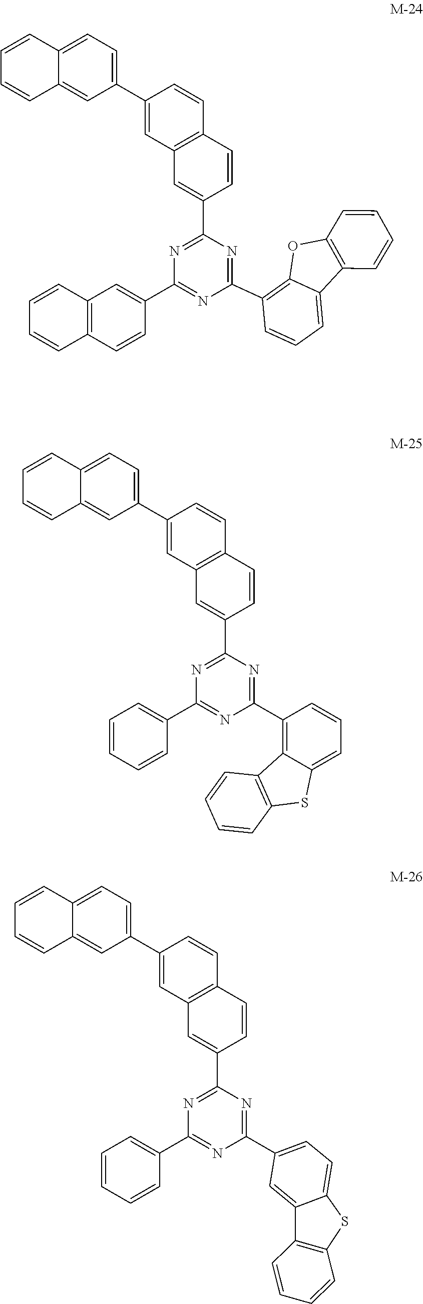 [SMILES: c1ccc2cc(-c3ccc4ccc(-c5nc(-c6ccc7ccccc7c6)nc(-c6cccc7c6oc6ccccc67)n5)cc4c3)ccc2c1 ; c1ccc(-c2nc(-c3ccc4ccc(-c5ccc6ccccc6c5)cc4c3)nc(-c3cccc4sc5ccccc5c34)n2)cc1 ; c1ccc(-c2nc(-c3ccc4ccc(-c5ccc6ccccc6c5)cc4c3)nc(-c3ccc4sc5ccccc5c4c3)n2)cc1]

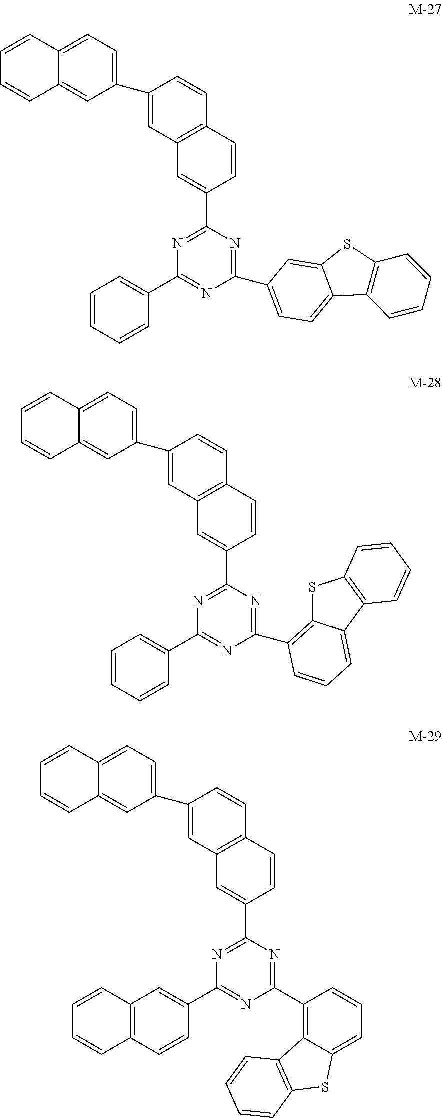

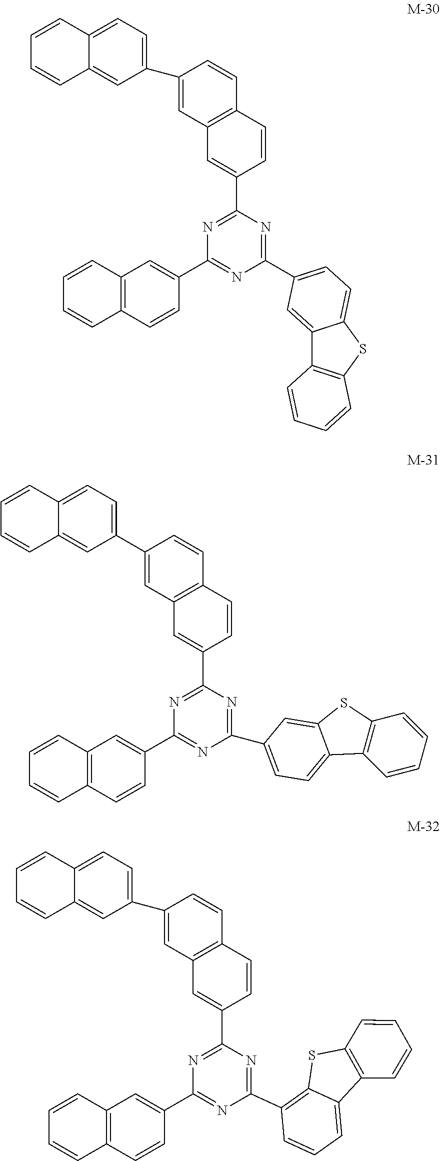 [SMILES: c1ccc2cc(-c3ccc4ccc(-c5nc(-c6ccc7ccccc7c6)nc(-c6ccc7sc8ccccc8c7c6)n5)cc4c3)ccc2c1 ; c1ccc2cc(-c3ccc4ccc(-c5nc(-c6ccc7ccccc7c6)nc(-c6ccc7c(c6)sc6ccccc67)n5)cc4c3)ccc2c1 ; c1ccc2cc(-c3ccc4ccc(-c5nc(-c6ccc7ccccc7c6)nc(-c6cccc7c6sc6ccccc67)n5)cc4c3)ccc2c1]

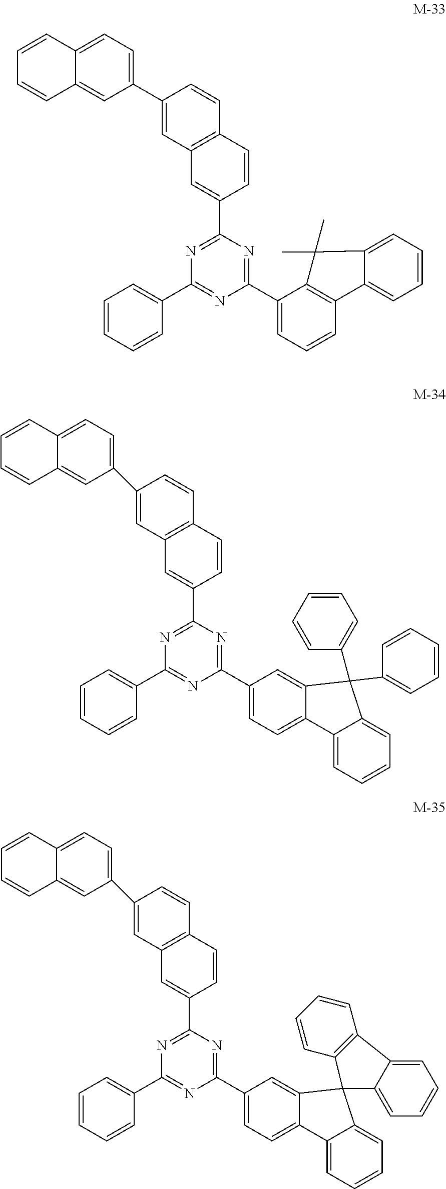 [SMILES: CC1(C)c2ccccc2-c2cccc(-c3nc(-c4ccccc4)nc(-c4ccc5ccc(-c6ccc7ccccc7c6)cc5c4)n3)c21 ; c1ccc(-c2nc(-c3ccc4c(c3)C(c3ccccc3)(c3ccccc3)c3ccccc3-4)nc(-c3ccc4ccc(-c5ccc6ccccc6c5)cc4c3)n2)cc1 ; c1ccc(-c2nc(-c3ccc4c(c3)C3(c5ccccc5-c5ccccc53)c3ccccc3-4)nc(-c3ccc4ccc(-c5ccc6ccccc6c5)cc4c3)n2)cc1]

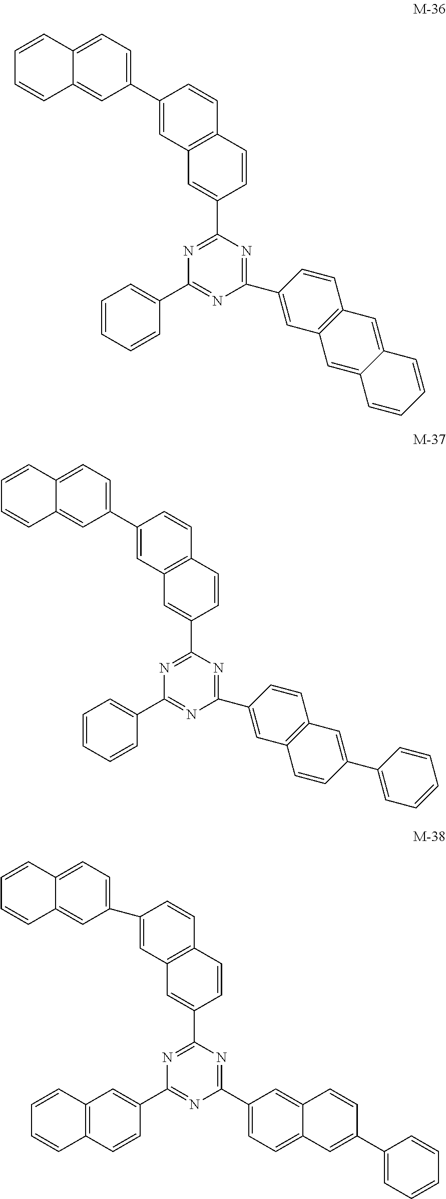 [SMILES: c1ccc(-c2nc(-c3ccc4ccc(-c5ccc6ccccc6c5)cc4c3)nc(-c3ccc4cc5ccccc5cc4c3)n2)cc1 ; c1ccc(-c2ccc3cc(-c4nc(-c5ccccc5)nc(-c5ccc6ccc(-c7ccc8ccccc8c7)cc6c5)n4)ccc3c2)cc1 ; c1ccc(-c2ccc3cc(-c4nc(-c5ccc6ccccc6c5)nc(-c5ccc6ccc(-c7ccc8ccccc8c7)cc6c5)n4)ccc3c2)cc1]

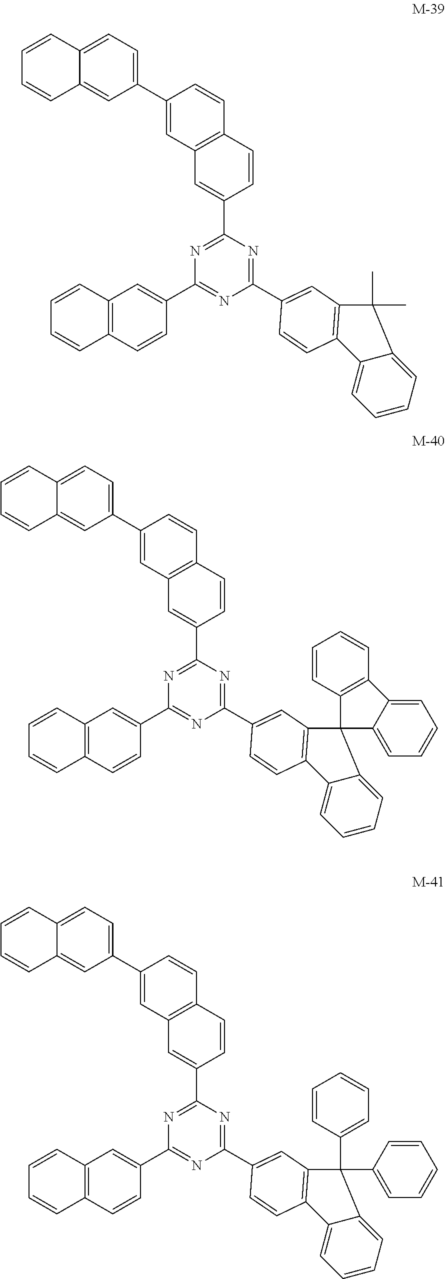 [SMILES: CC1(C)c2ccccc2-c2ccc(-c3nc(-c4ccc5ccccc5c4)nc(-c4ccc5ccc(-c6ccc7ccccc7c6)cc5c4)n3)cc21 ; c1ccc2c(c1)-c1ccccc1C21c2ccccc2-c2ccc(-c3nc(-c4ccc5ccccc5c4)nc(-c4ccc5ccc(-c6ccc7ccccc7c6)cc5c4)n3)cc21 ; c1ccc(C2(c3ccccc3)c3ccccc3-c3ccc(-c4nc(-c5ccc6ccccc6c5)nc(-c5ccc6ccc(-c7ccc8ccccc8c7)cc6c5)n4)cc32)cc1]

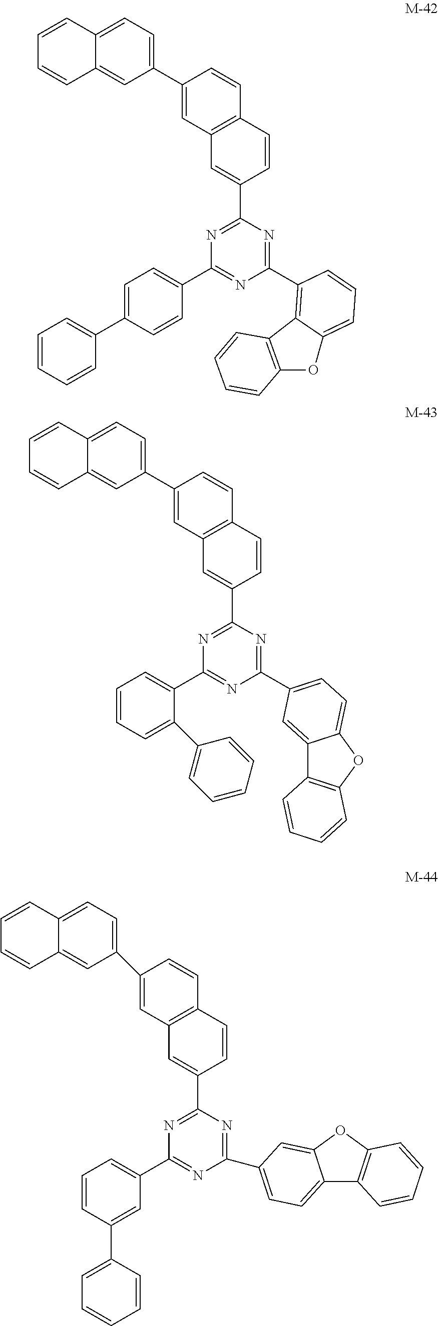

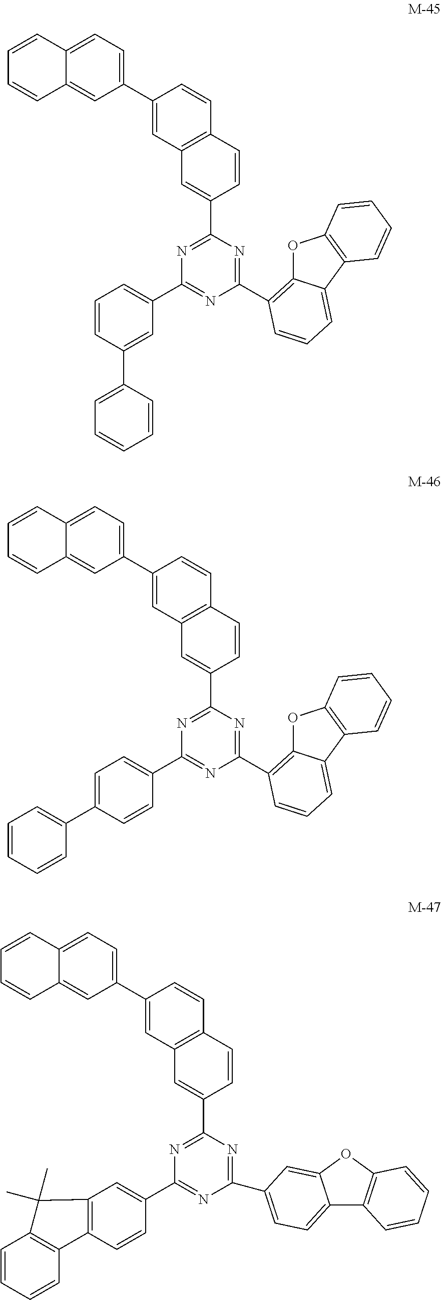 [SMILES: c1ccc(-c2cccc(-c3nc(-c4ccc5ccc(-c6ccc7ccccc7c6)cc5c4)nc(-c4cccc5c4oc4ccccc45)n3)c2)cc1 ; c1ccc(-c2ccc(-c3nc(-c4ccc5ccc(-c6ccc7ccccc7c6)cc5c4)nc(-c4cccc5c4oc4ccccc45)n3)cc2)cc1 ; CC1(C)c2ccccc2-c2ccc(-c3nc(-c4ccc5ccc(-c6ccc7ccccc7c6)cc5c4)nc(-c4ccc5c(c4)oc4ccccc45)n3)cc21]

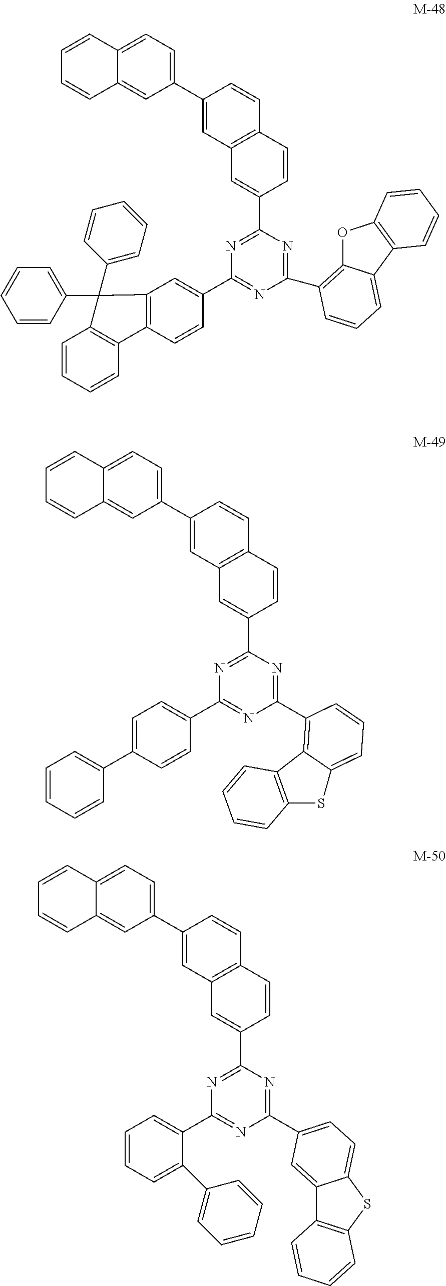 [SMILES: c1ccc(C2(c3ccccc3)c3ccccc3-c3ccc(-c4nc(-c5ccc6ccc(-c7ccc8ccccc8c7)cc6c5)nc(-c5cccc6c5oc5ccccc56)n4)cc32)cc1 ; c1ccc(-c2ccc(-c3nc(-c4ccc5ccc(-c6ccc7ccccc7c6)cc5c4)nc(-c4cccc5sc6ccccc6c45)n3)cc2)cc1 ; c1ccc(-c2ccccc2-c2nc(-c3ccc4ccc(-c5ccc6ccccc6c5)cc4c3)nc(-c3ccc4sc5ccccc5c4c3)n2)cc1]

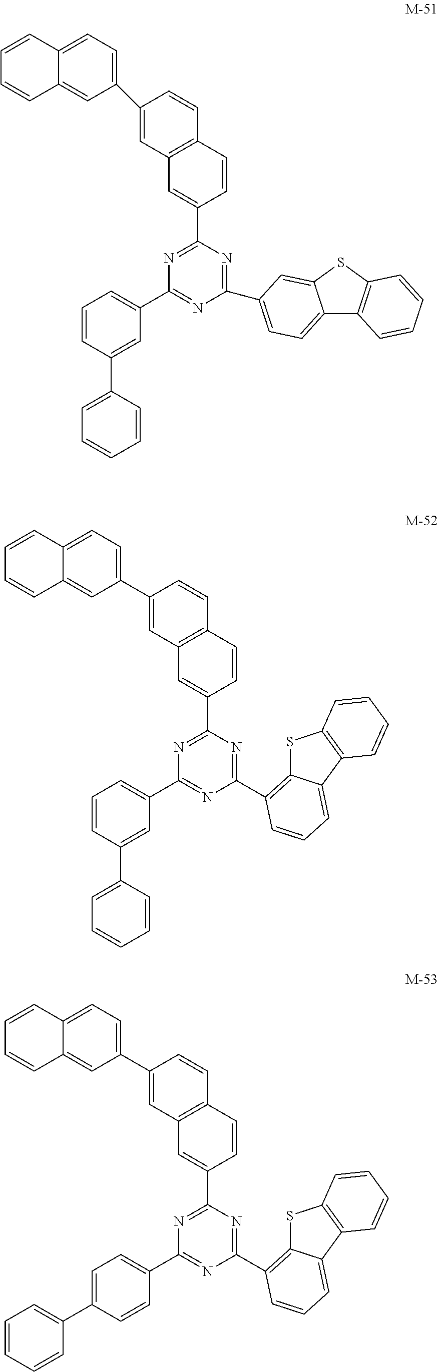 [SMILES: c1ccc(-c2cccc(-c3nc(-c4ccc5ccc(-c6ccc7ccccc7c6)cc5c4)nc(-c4ccc5c(c4)sc4ccccc45)n3)c2)cc1 ; c1ccc(-c2cccc(-c3nc(-c4ccc5ccc(-c6ccc7ccccc7c6)cc5c4)nc(-c4cccc5c4sc4ccccc45)n3)c2)cc1 ; c1ccc(-c2ccc(-c3nc(-c4ccc5ccc(-c6ccc7ccccc7c6)cc5c4)nc(-c4cccc5c4sc4ccccc45)n3)cc2)cc1]

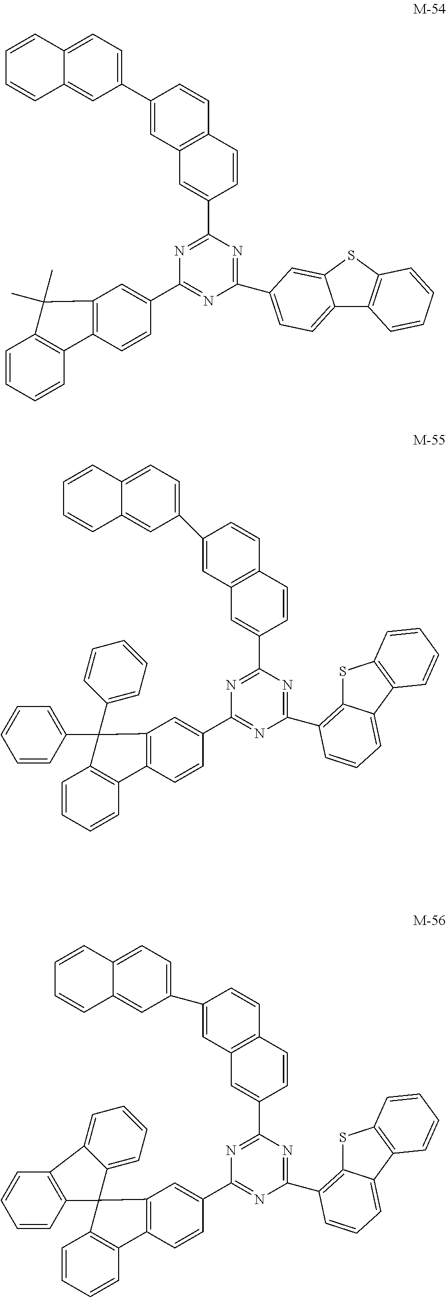 [SMILES: CC1(C)c2ccccc2-c2ccc(-c3nc(-c4ccc5ccc(-c6ccc7ccccc7c6)cc5c4)nc(-c4ccc5c(c4)sc4ccccc45)n3)cc21 ; c1ccc(C2(c3ccccc3)c3ccccc3-c3ccc(-c4nc(-c5ccc6ccc(-c7ccc8ccccc8c7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6ccc7ccccc7c6)cc5c4)nc(-c4cccc5c4sc4ccccc45)n3)cc21]

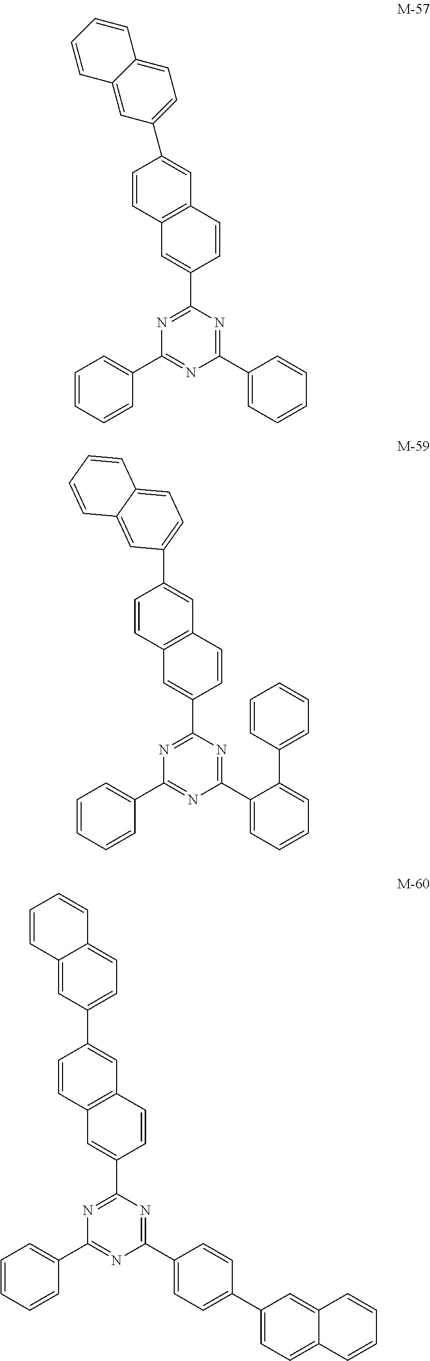

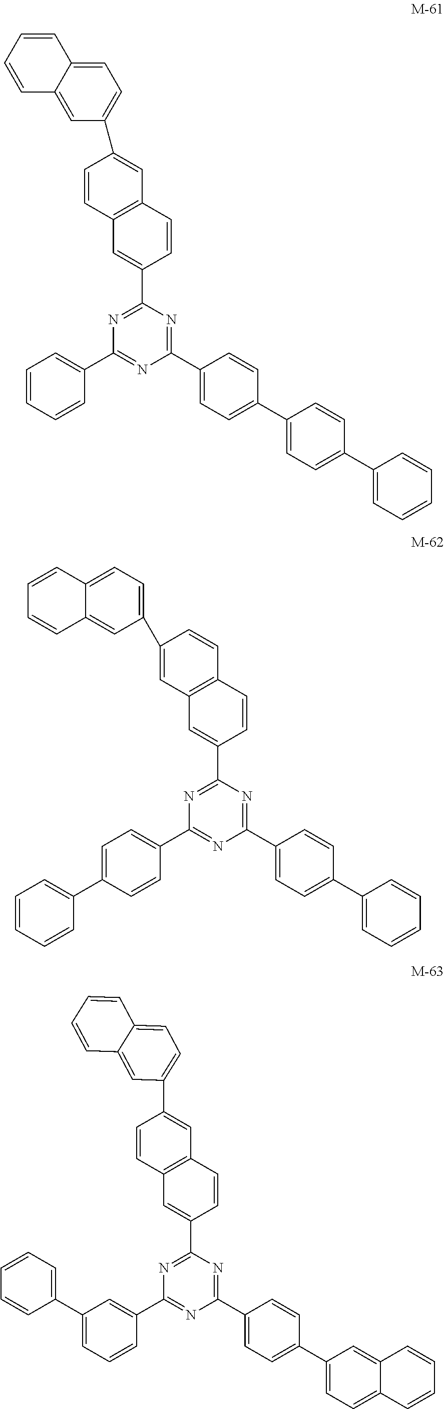 [SMILES: c1ccc(-c2ccc(-c3ccc(-c4nc(-c5ccccc5)nc(-c5ccc6cc(-c7ccc8ccccc8c7)ccc6c5)n4)cc3)cc2)cc1 ; c1ccc(-c2ccc(-c3nc(-c4ccc(-c5ccccc5)cc4)nc(-c4ccc5ccc(-c6ccc7ccccc7c6)cc5c4)n3)cc2)cc1 ; c1ccc(-c2cccc(-c3nc(-c4ccc(-c5ccc6ccccc6c5)cc4)nc(-c4ccc5cc(-c6ccc7ccccc7c6)ccc5c4)n3)c2)cc1]

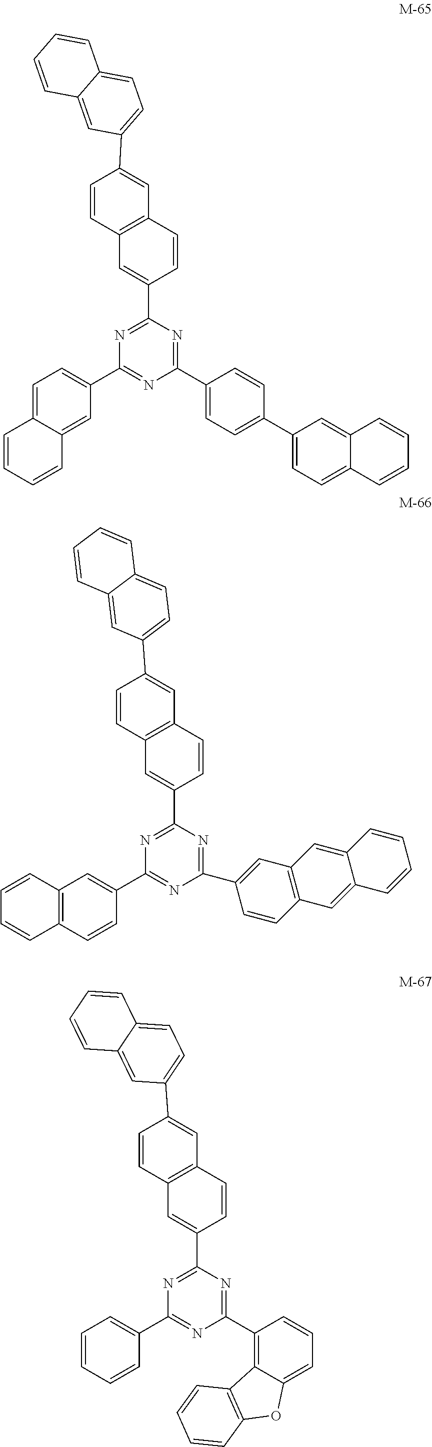 [SMILES: c1ccc2cc(-c3ccc(-c4nc(-c5ccc6ccccc6c5)nc(-c5ccc6cc(-c7ccc8ccccc8c7)ccc6c5)n4)cc3)ccc2c1 ; c1ccc2cc(-c3ccc4cc(-c5nc(-c6ccc7ccccc7c6)nc(-c6ccc7cc8ccccc8cc7c6)n5)ccc4c3)ccc2c1 ; c1ccc(-c2nc(-c3ccc4cc(-c5ccc6ccccc6c5)ccc4c3)nc(-c3cccc4oc5ccccc5c34)n2)cc1]

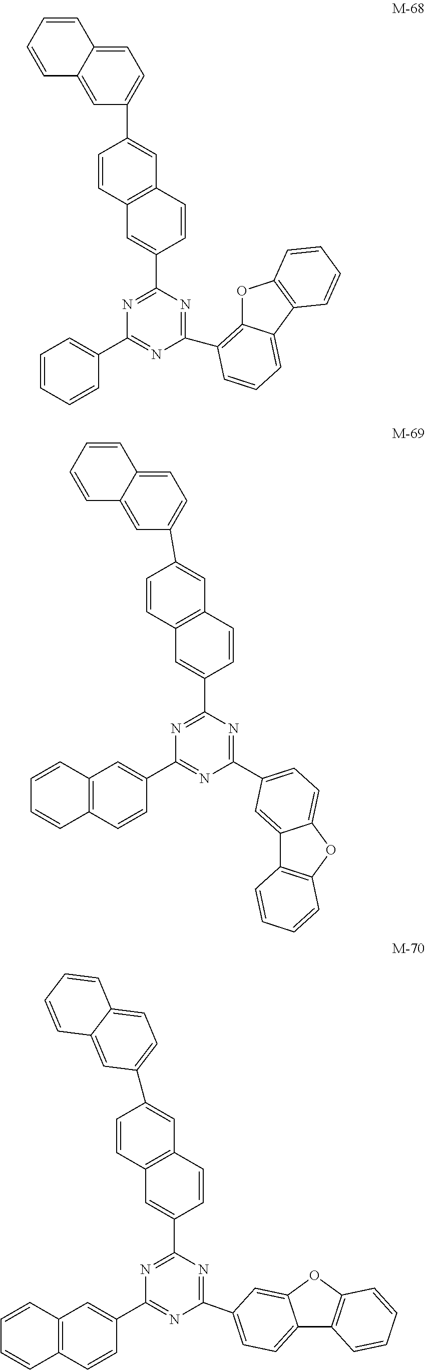

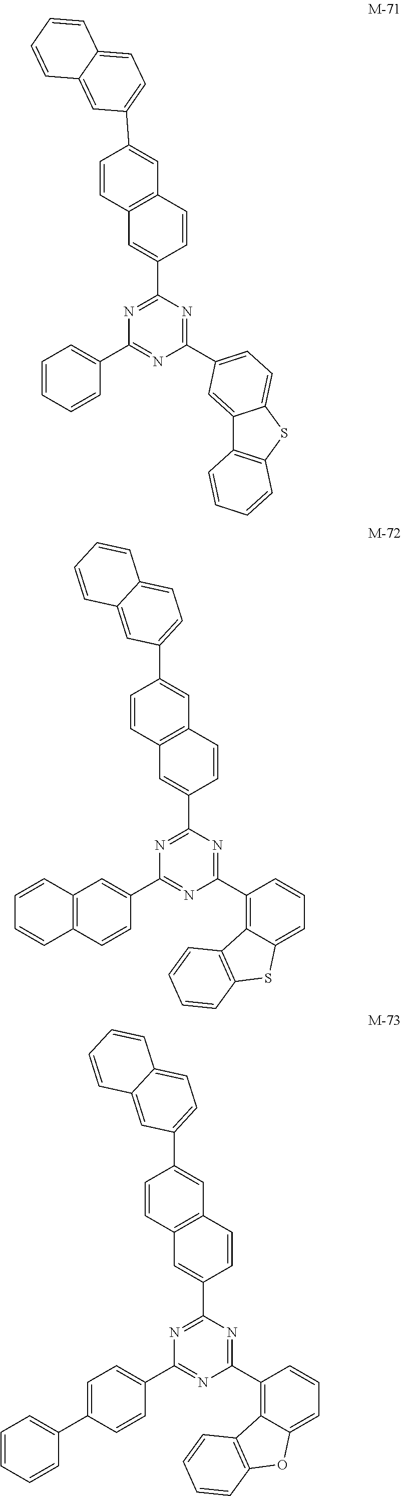 [SMILES: c1ccc(-c2nc(-c3ccc4cc(-c5ccc6ccccc6c5)ccc4c3)nc(-c3ccc4sc5ccccc5c4c3)n2)cc1 ; c1ccc2cc(-c3ccc4cc(-c5nc(-c6ccc7ccccc7c6)nc(-c6cccc7sc8ccccc8c67)n5)ccc4c3)ccc2c1 ; c1ccc(-c2ccc(-c3nc(-c4ccc5cc(-c6ccc7ccccc7c6)ccc5c4)nc(-c4cccc5oc6ccccc6c45)n3)cc2)cc1]

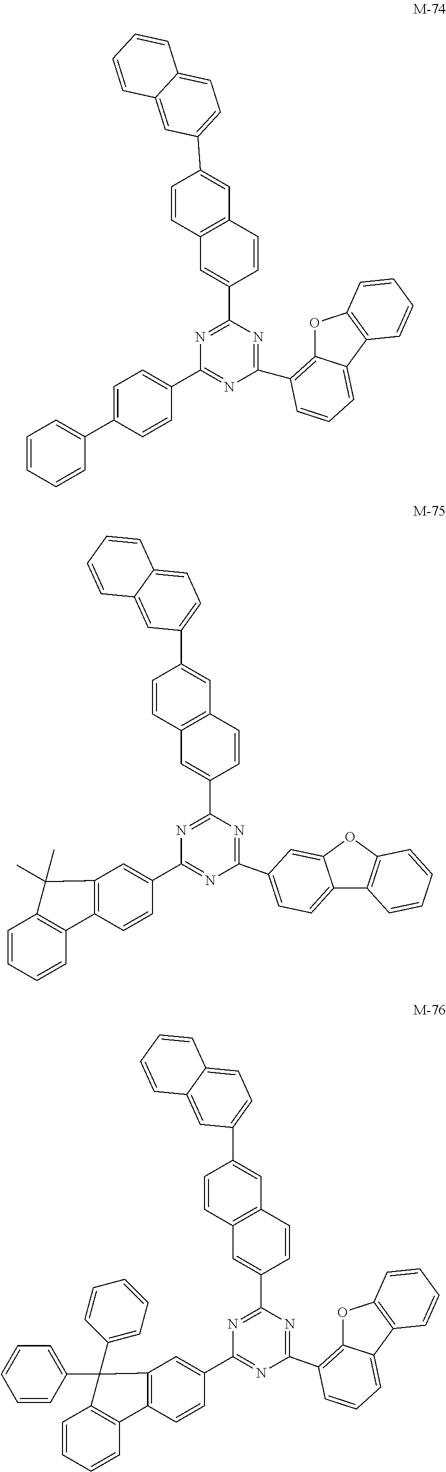 [SMILES: c1ccc(-c2ccc(-c3nc(-c4ccc5cc(-c6ccc7ccccc7c6)ccc5c4)nc(-c4cccc5c4oc4ccccc45)n3)cc2)cc1 ; CC1(C)c2ccccc2-c2ccc(-c3nc(-c4ccc5cc(-c6ccc7ccccc7c6)ccc5c4)nc(-c4ccc5c(c4)oc4ccccc45)n3)cc21 ; c1ccc(C2(c3ccccc3)c3ccccc3-c3ccc(-c4nc(-c5ccc6cc(-c7ccc8ccccc8c7)ccc6c5)nc(-c5cccc6c5oc5ccccc56)n4)cc32)cc1]

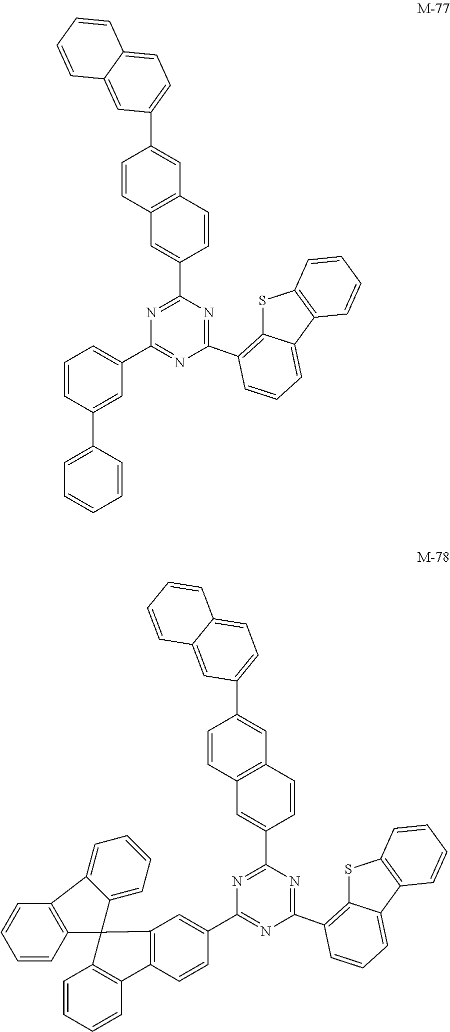 [SMILES: c1ccc(-c2cccc(-c3nc(-c4ccc5cc(-c6ccc7ccccc7c6)ccc5c4)nc(-c4cccc5c4sc4ccccc45)n3)c2)cc1 ; c1ccc2c(c1)-c1ccccc1C21c2ccccc2-c2ccc(-c3nc(-c4ccc5cc(-c6ccc7ccccc7c6)ccc5c4)nc(-c4cccc5c4sc4ccccc45)n3)cc21]

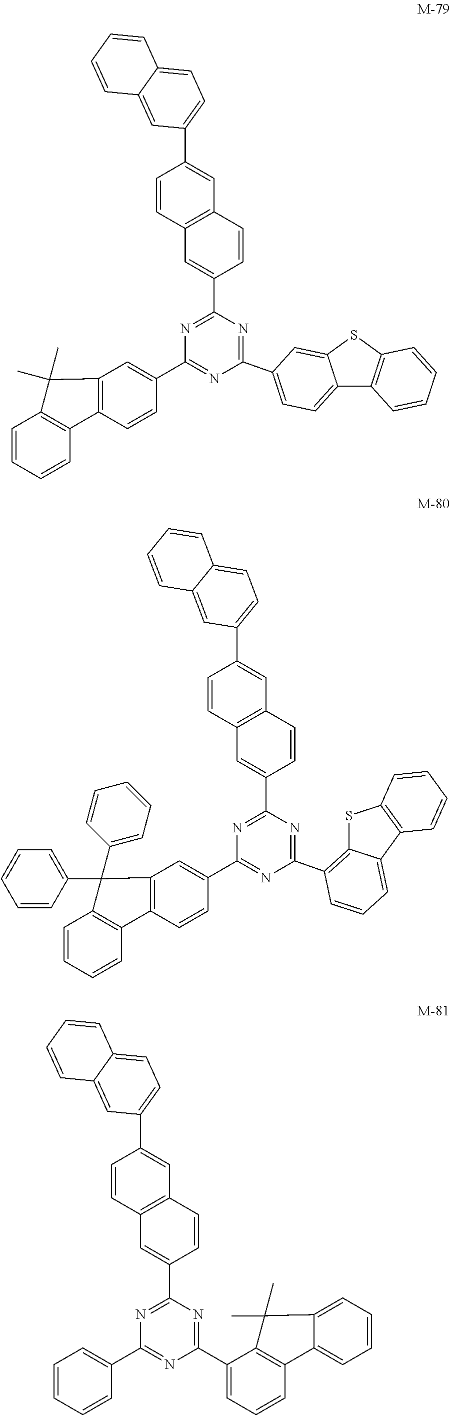

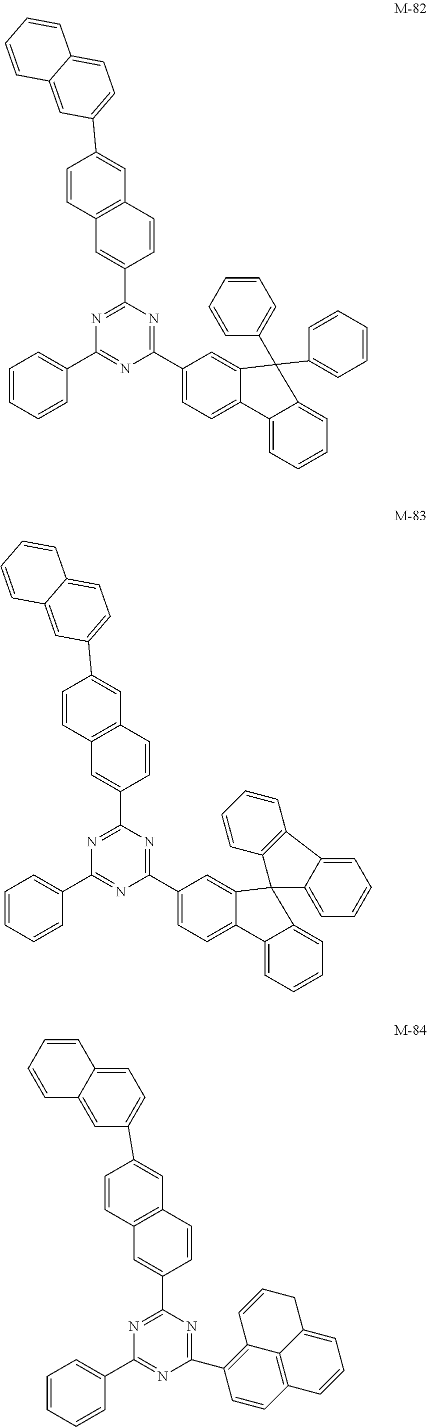 [SMILES: c1ccc(-c2nc(-c3ccc4c(c3)C(c3ccccc3)(c3ccccc3)c3ccccc3-4)nc(-c3ccc4cc(-c5ccc6ccccc6c5)ccc4c3)n2)cc1 ; c1ccc(-c2nc(-c3ccc4c(c3)C3(c5ccccc5-c5ccccc53)c3ccccc3-4)nc(-c3ccc4cc(-c5ccc6ccccc6c5)ccc4c3)n2)cc1 ; C1=Cc2c(-c3nc(-c4ccccc4)nc(-c4ccc5cc(-c6ccc7ccccc7c6)ccc5c4)n3)ccc3cccc(c23)C1]

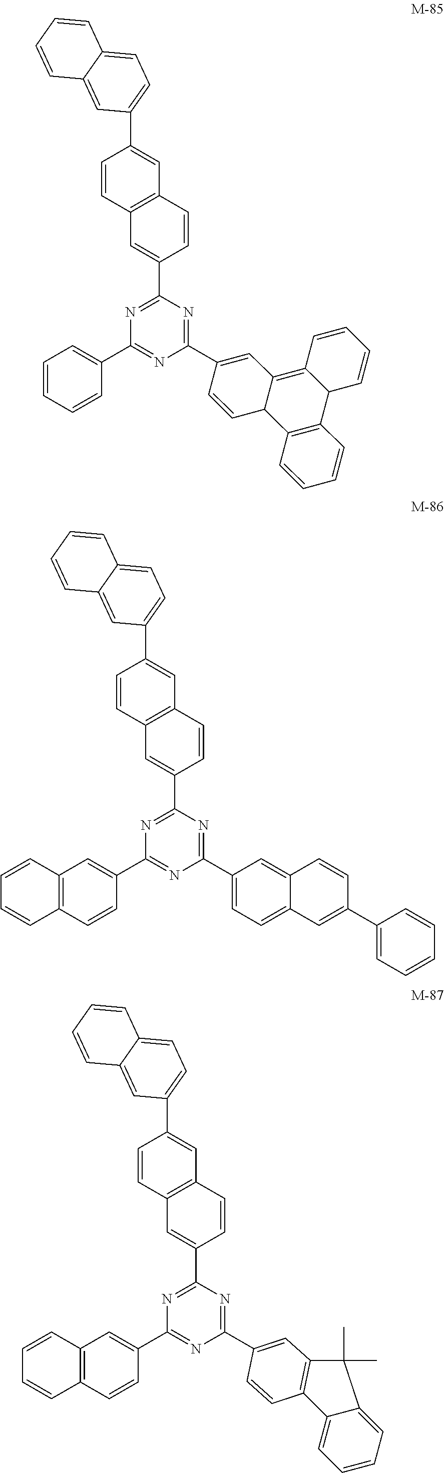 [SMILES: C1=CC2=C3C=C(c4nc(-c5ccccc5)nc(-c5ccc6cc(-c7ccc8ccccc8c7)ccc6c5)n4)C=CC3c3ccccc3C2C=C1 ; c1ccc(-c2ccc3cc(-c4nc(-c5ccc6ccccc6c5)nc(-c5ccc6cc(-c7ccc8ccccc8c7)ccc6c5)n4)ccc3c2)cc1 ; CC1(C)c2ccccc2-c2ccc(-c3nc(-c4ccc5ccccc5c4)nc(-c4ccc5cc(-c6ccc7ccccc7c6)ccc5c4)n3)cc21]

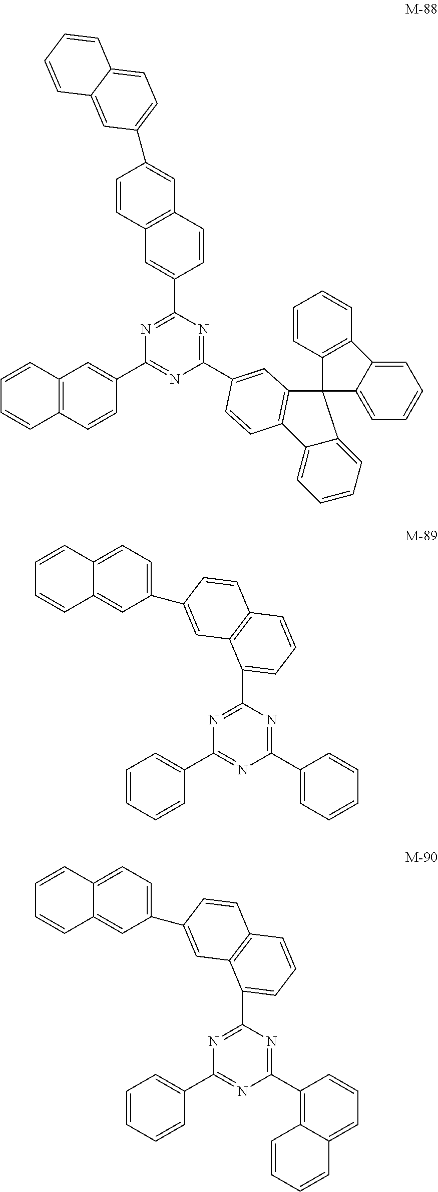 [SMILES: c1ccc2c(c1)-c1ccccc1C21c2ccccc2-c2ccc(-c3nc(-c4ccc5ccccc5c4)nc(-c4ccc5cc(-c6ccc7ccccc7c6)ccc5c4)n3)cc21 ; c1ccc(-c2nc(-c3ccccc3)nc(-c3cccc4ccc(-c5ccc6ccccc6c5)cc34)n2)cc1 ; c1ccc(-c2nc(-c3cccc4ccccc34)nc(-c3cccc4ccc(-c5ccc6ccccc6c5)cc34)n2)cc1]

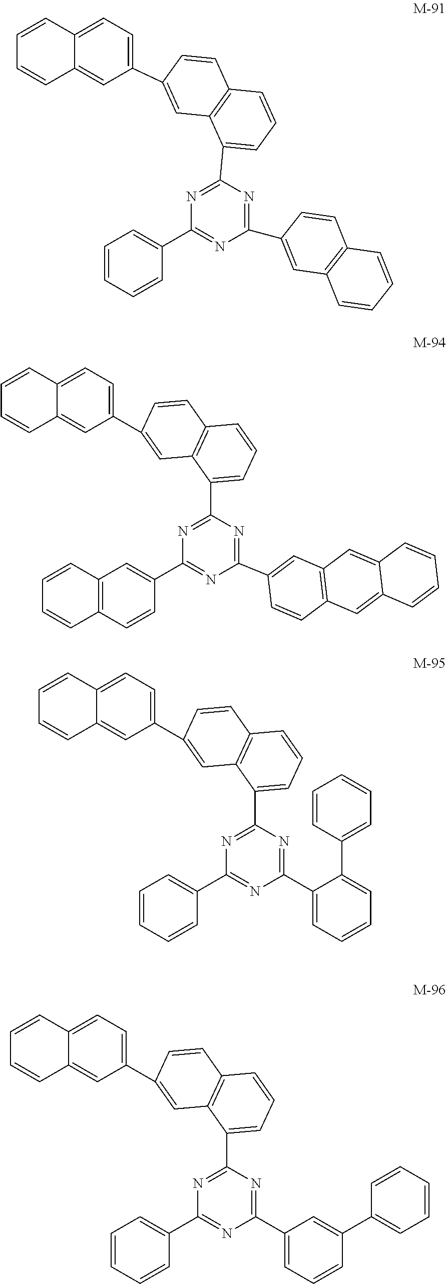

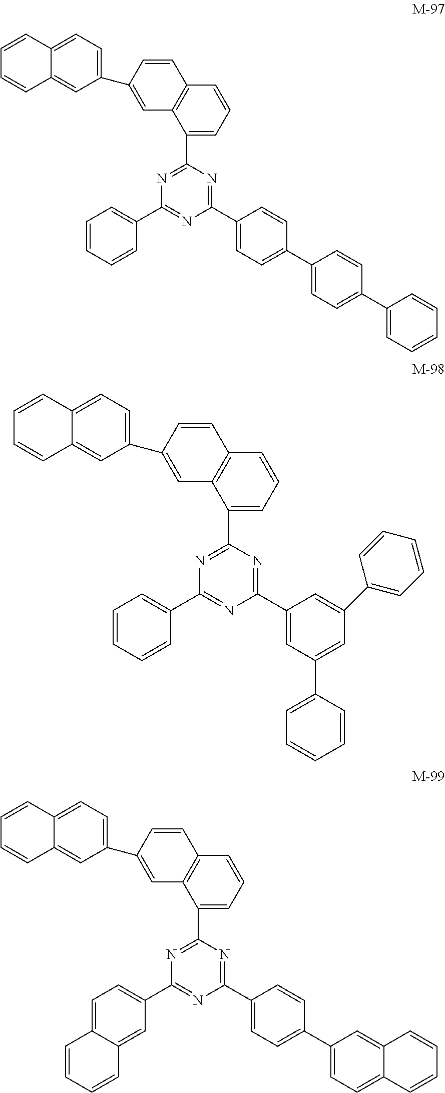 [SMILES: c1ccc(-c2ccc(-c3ccc(-c4nc(-c5ccccc5)nc(-c5cccc6ccc(-c7ccc8ccccc8c7)cc56)n4)cc3)cc2)cc1 ; c1ccc(-c2cc(-c3ccccc3)cc(-c3nc(-c4ccccc4)nc(-c4cccc5ccc(-c6ccc7ccccc7c6)cc45)n3)c2)cc1 ; c1ccc2cc(-c3ccc(-c4nc(-c5ccc6ccccc6c5)nc(-c5cccc6ccc(-c7ccc8ccccc8c7)cc56)n4)cc3)ccc2c1]

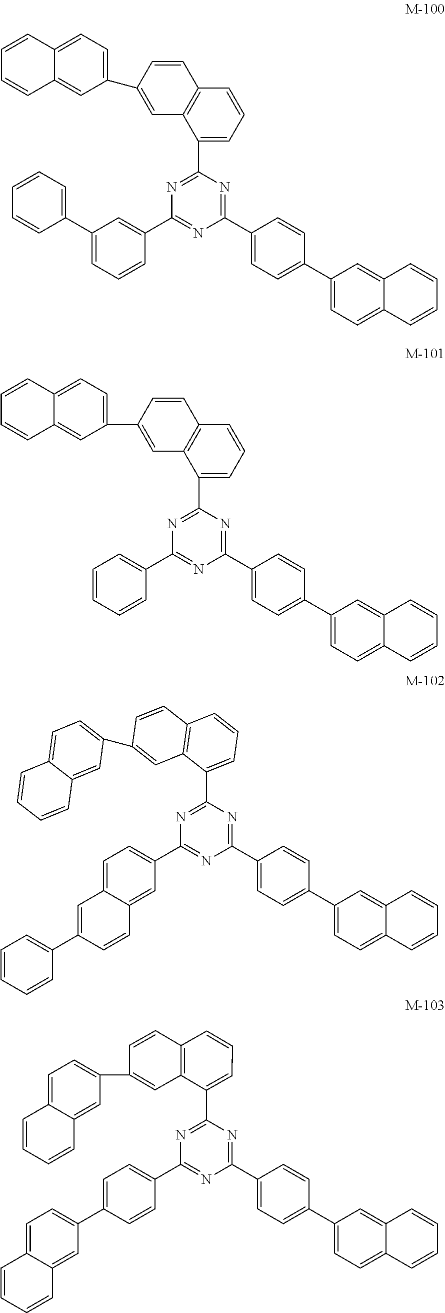 [SMILES: c1ccc(-c2cccc(-c3nc(-c4ccc(-c5ccc6ccccc6c5)cc4)nc(-c4cccc5ccc(-c6ccc7ccccc7c6)cc45)n3)c2)cc1 ; c1ccc(-c2nc(-c3ccc(-c4ccc5ccccc5c4)cc3)nc(-c3cccc4ccc(-c5ccc6ccccc6c5)cc34)n2)cc1 ; c1ccc(-c2ccc3cc(-c4nc(-c5ccc(-c6ccc7ccccc7c6)cc5)nc(-c5cccc6ccc(-c7ccc8ccccc8c7)cc56)n4)ccc3c2)cc1 ; c1ccc2cc(-c3ccc(-c4nc(-c5ccc(-c6ccc7ccccc7c6)cc5)nc(-c5cccc6ccc(-c7ccc8ccccc8c7)cc56)n4)cc3)ccc2c1]

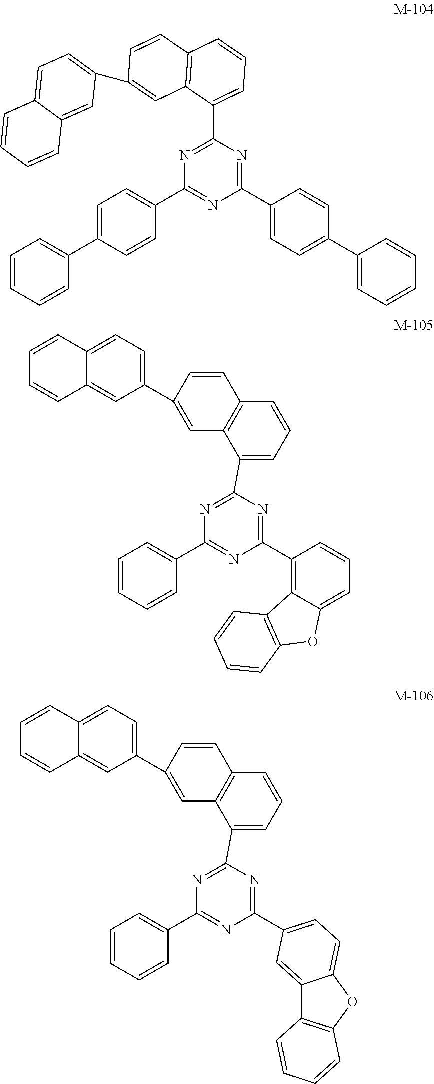

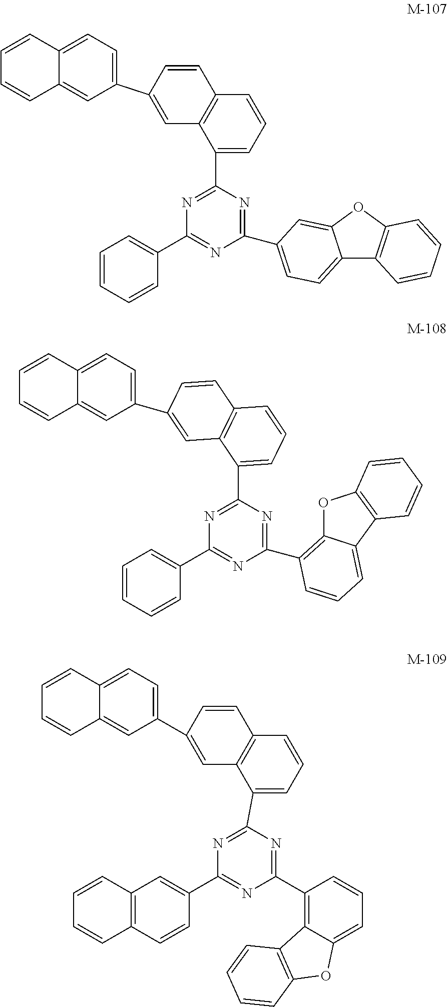 [SMILES: c1ccc(-c2nc(-c3ccc4c(c3)oc3ccccc34)nc(-c3cccc4ccc(-c5ccc6ccccc6c5)cc34)n2)cc1 ; c1ccc(-c2nc(-c3cccc4ccc(-c5ccc6ccccc6c5)cc34)nc(-c3cccc4c3oc3ccccc34)n2)cc1 ; c1ccc2cc(-c3ccc4cccc(-c5nc(-c6ccc7ccccc7c6)nc(-c6cccc7oc8ccccc8c67)n5)c4c3)ccc2c1]

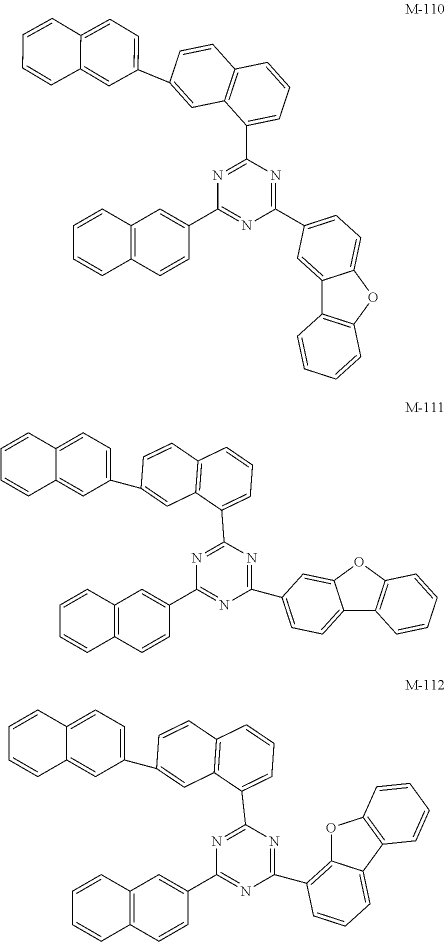 [SMILES: c1ccc2cc(-c3ccc4cccc(-c5nc(-c6ccc7ccccc7c6)nc(-c6ccc7oc8ccccc8c7c6)n5)c4c3)ccc2c1 ; c1ccc2cc(-c3ccc4cccc(-c5nc(-c6ccc7ccccc7c6)nc(-c6ccc7c(c6)oc6ccccc67)n5)c4c3)ccc2c1 ; c1ccc2cc(-c3ccc4cccc(-c5nc(-c6ccc7ccccc7c6)nc(-c6cccc7c6oc6ccccc67)n5)c4c3)ccc2c1]

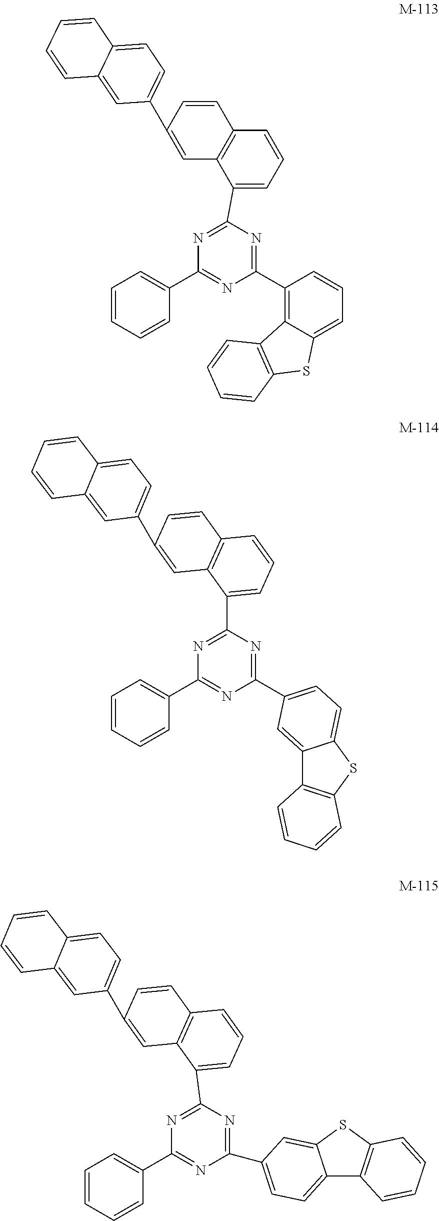 [SMILES: c1ccc(-c2nc(-c3cccc4ccc(-c5ccc6ccccc6c5)cc34)nc(-c3cccc4sc5ccccc5c34)n2)cc1 ; c1ccc(-c2nc(-c3ccc4sc5ccccc5c4c3)nc(-c3cccc4ccc(-c5ccc6ccccc6c5)cc34)n2)cc1 ; c1ccc(-c2nc(-c3ccc4c(c3)sc3ccccc34)nc(-c3cccc4ccc(-c5ccc6ccccc6c5)cc34)n2)cc1]

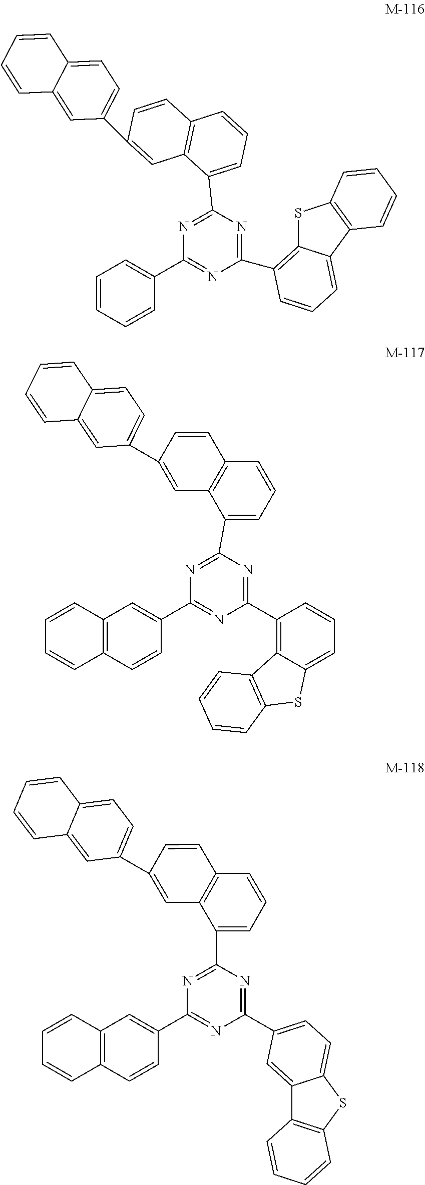 [SMILES: c1ccc(-c2nc(-c3cccc4ccc(-c5ccc6ccccc6c5)cc34)nc(-c3cccc4c3sc3ccccc34)n2)cc1 ; c1ccc2cc(-c3ccc4cccc(-c5nc(-c6ccc7ccccc7c6)nc(-c6cccc7sc8ccccc8c67)n5)c4c3)ccc2c1 ; c1ccc2cc(-c3ccc4cccc(-c5nc(-c6ccc7ccccc7c6)nc(-c6ccc7sc8ccccc8c7c6)n5)c4c3)ccc2c1]

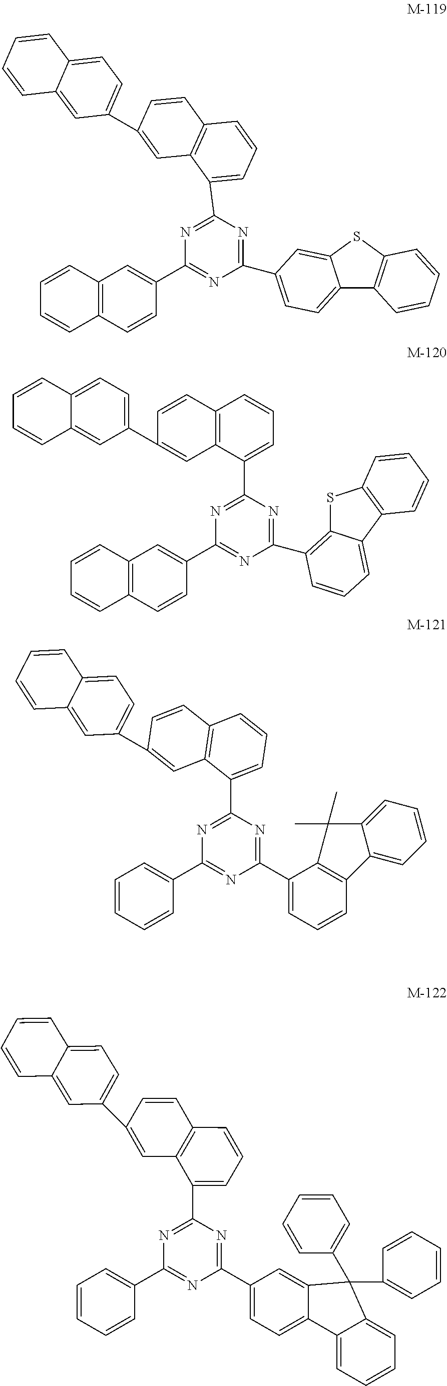 [SMILES: c1ccc2cc(-c3ccc4cccc(-c5nc(-c6ccc7ccccc7c6)nc(-c6ccc7c(c6)sc6ccccc67)n5)c4c3)ccc2c1 ; c1ccc2cc(-c3ccc4cccc(-c5nc(-c6ccc7ccccc7c6)nc(-c6cccc7c6sc6ccccc67)n5)c4c3)ccc2c1 ; CC1(C)c2ccccc2-c2cccc(-c3nc(-c4ccccc4)nc(-c4cccc5ccc(-c6ccc7ccccc7c6)cc45)n3)c21 ; c1ccc(-c2nc(-c3ccc4c(c3)C(c3ccccc3)(c3ccccc3)c3ccccc3-4)nc(-c3cccc4ccc(-c5ccc6ccccc6c5)cc34)n2)cc1]

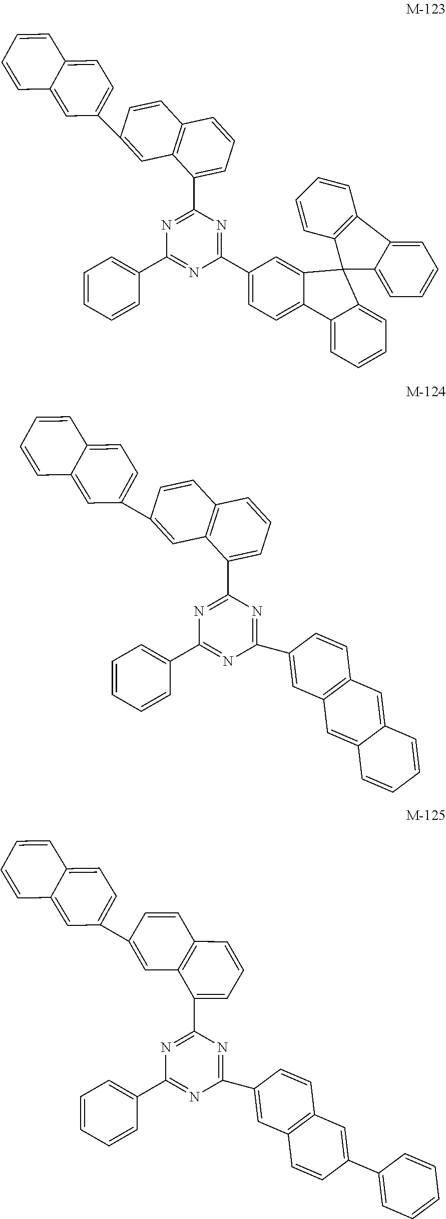 [SMILES: c1ccc(-c2nc(-c3ccc4c(c3)C3(c5ccccc5-c5ccccc53)c3ccccc3-4)nc(-c3cccc4ccc(-c5ccc6ccccc6c5)cc34)n2)cc1 ; c1ccc(-c2nc(-c3ccc4cc5ccccc5cc4c3)nc(-c3cccc4ccc(-c5ccc6ccccc6c5)cc34)n2)cc1 ; c1ccc(-c2ccc3cc(-c4nc(-c5ccccc5)nc(-c5cccc6ccc(-c7ccc8ccccc8c7)cc56)n4)ccc3c2)cc1]

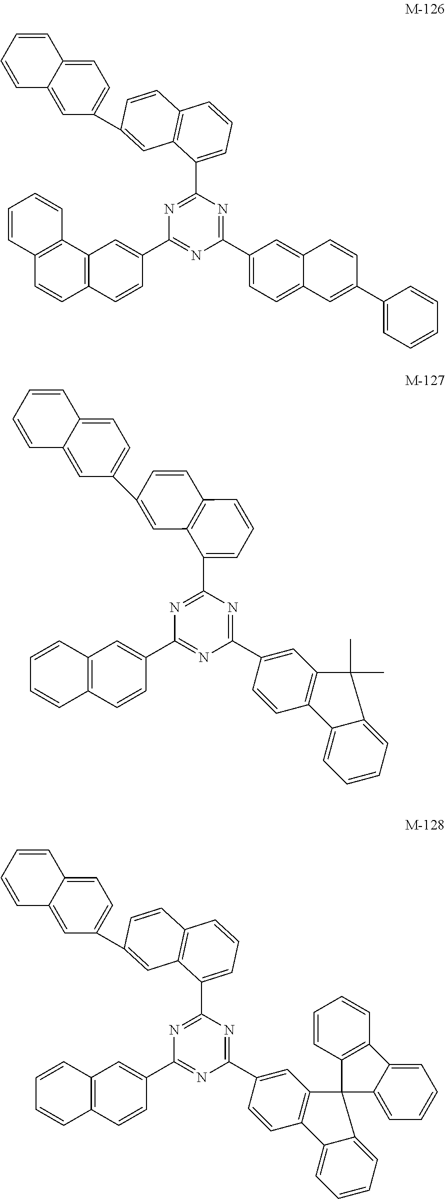 [SMILES: c1ccc(-c2ccc3cc(-c4nc(-c5ccc6ccc7ccccc7c6c5)nc(-c5cccc6ccc(-c7ccc8ccccc8c7)cc56)n4)ccc3c2)cc1 ; CC1(C)c2ccccc2-c2ccc(-c3nc(-c4ccc5ccccc5c4)nc(-c4cccc5ccc(-c6ccc7ccccc7c6)cc45)n3)cc21 ; c1ccc2c(c1)-c1ccccc1C21c2ccccc2-c2ccc(-c3nc(-c4ccc5ccccc5c4)nc(-c4cccc5ccc(-c6ccc7ccccc7c6)cc45)n3)cc21]

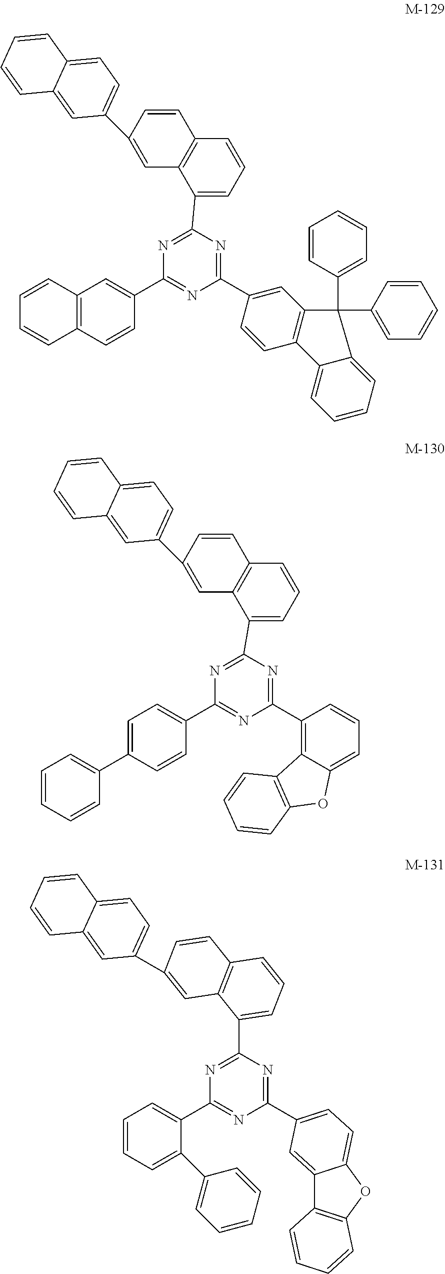 [SMILES: c1ccc(C2(c3ccccc3)c3ccccc3-c3ccc(-c4nc(-c5ccc6ccccc6c5)nc(-c5cccc6ccc(-c7ccc8ccccc8c7)cc56)n4)cc32)cc1 ; c1ccc(-c2ccc(-c3nc(-c4cccc5ccc(-c6ccc7ccccc7c6)cc45)nc(-c4cccc5oc6ccccc6c45)n3)cc2)cc1 ; c1ccc(-c2ccccc2-c2nc(-c3ccc4oc5ccccc5c4c3)nc(-c3cccc4ccc(-c5ccc6ccccc6c5)cc34)n2)cc1]

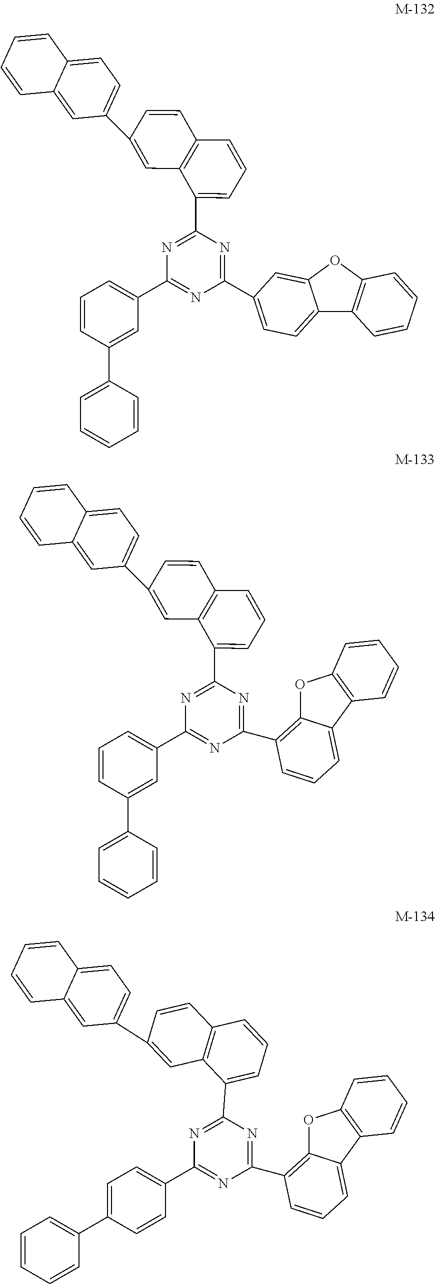 [SMILES: c1ccc(-c2cccc(-c3nc(-c4ccc5c(c4)oc4ccccc45)nc(-c4cccc5ccc(-c6ccc7ccccc7c6)cc45)n3)c2)cc1 ; c1ccc(-c2cccc(-c3nc(-c4cccc5ccc(-c6ccc7ccccc7c6)cc45)nc(-c4cccc5c4oc4ccccc45)n3)c2)cc1 ; c1ccc(-c2ccc(-c3nc(-c4cccc5ccc(-c6ccc7ccccc7c6)cc45)nc(-c4cccc5c4oc4ccccc45)n3)cc2)cc1]

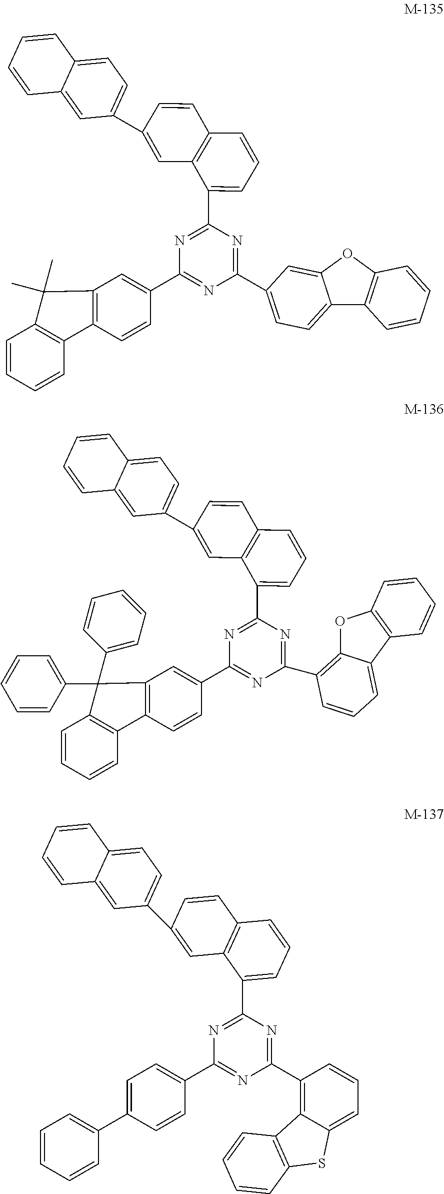

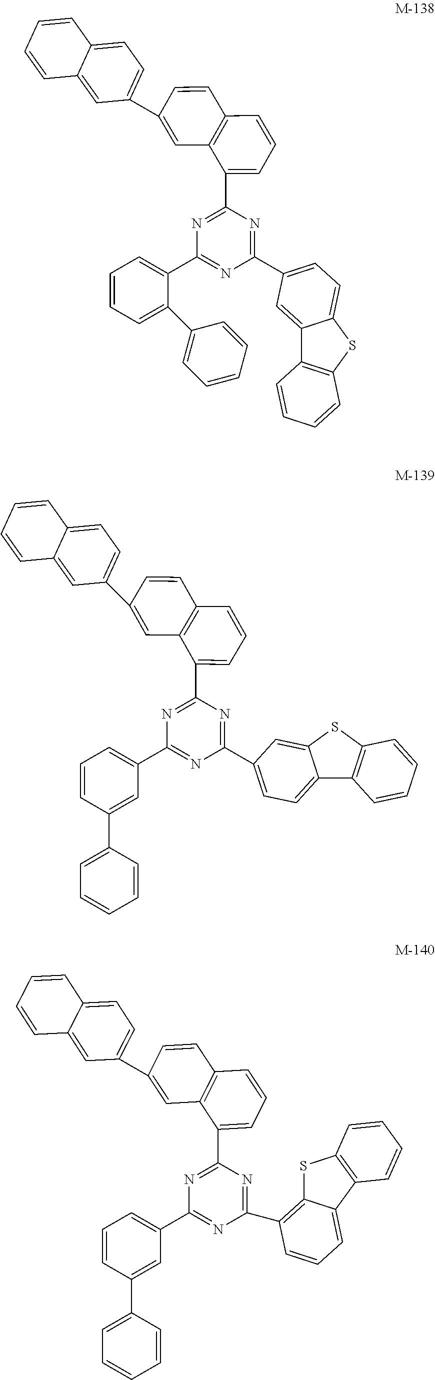 [SMILES: c1ccc(-c2ccccc2-c2nc(-c3ccc4sc5ccccc5c4c3)nc(-c3cccc4ccc(-c5ccc6ccccc6c5)cc34)n2)cc1 ; c1ccc(-c2cccc(-c3nc(-c4ccc5c(c4)sc4ccccc45)nc(-c4cccc5ccc(-c6ccc7ccccc7c6)cc45)n3)c2)cc1 ; c1ccc(-c2cccc(-c3nc(-c4cccc5ccc(-c6ccc7ccccc7c6)cc45)nc(-c4cccc5c4sc4ccccc45)n3)c2)cc1]

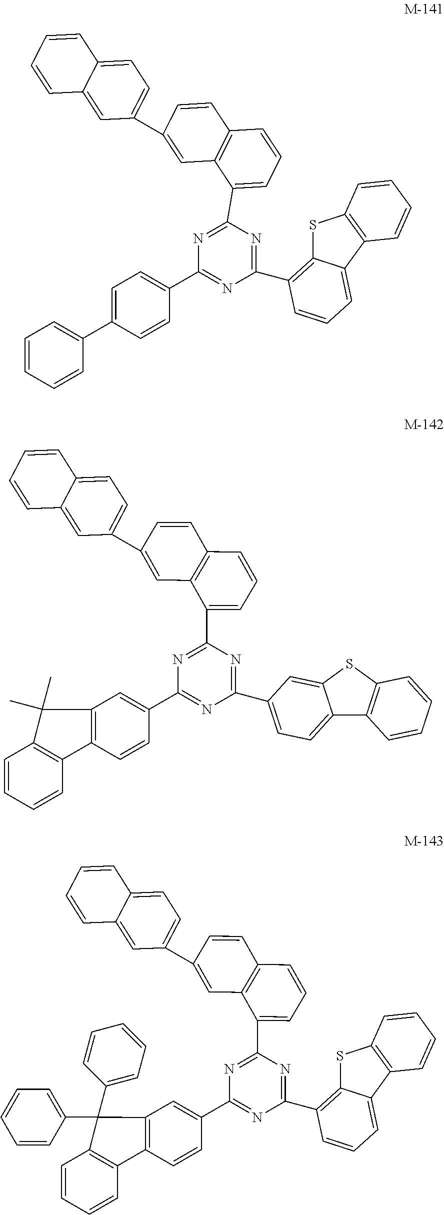 [SMILES: c1ccc(-c2ccc(-c3nc(-c4cccc5ccc(-c6ccc7ccccc7c6)cc45)nc(-c4cccc5c4sc4ccccc45)n3)cc2)cc1 ; CC1(C)c2ccccc2-c2ccc(-c3nc(-c4ccc5c(c4)sc4ccccc45)nc(-c4cccc5ccc(-c6ccc7ccccc7c6)cc45)n3)cc21 ; c1ccc(C2(c3ccccc3)c3ccccc3-c3ccc(-c4nc(-c5cccc6ccc(-c7ccc8ccccc8c7)cc56)nc(-c5cccc6c5sc5ccccc56)n4)cc32)cc1]

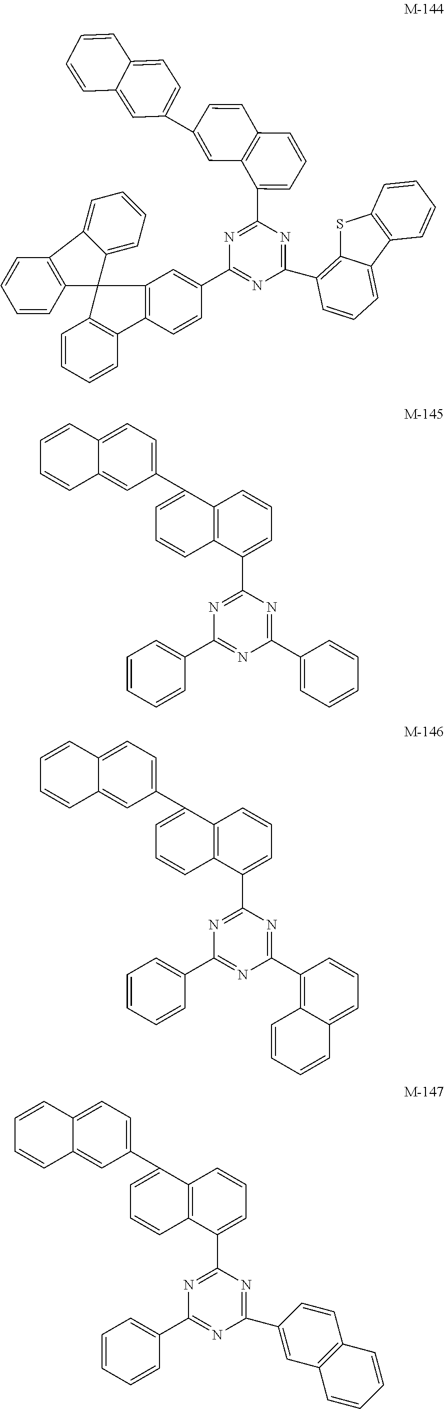

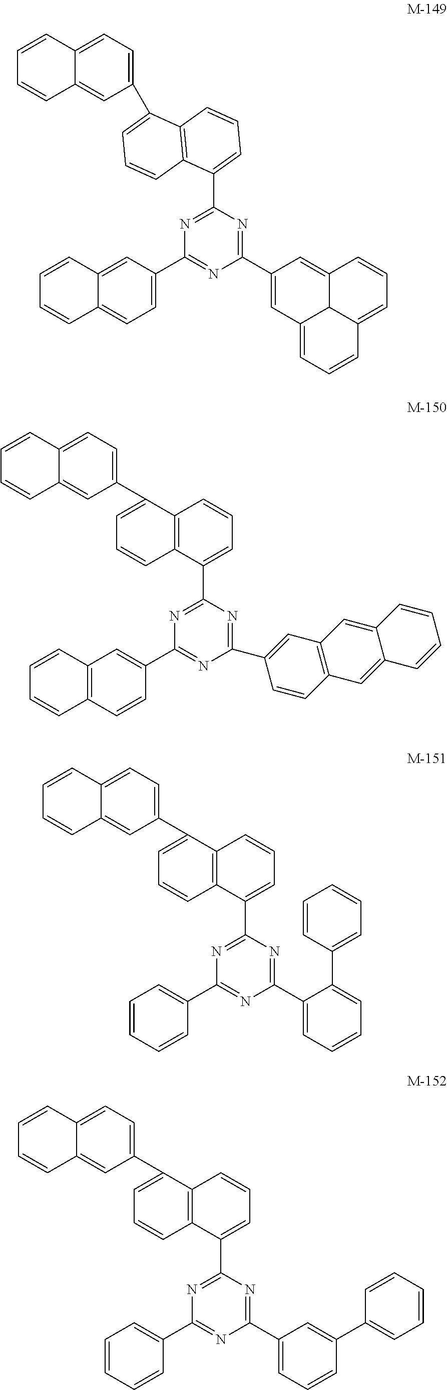 [SMILES: C1=CC2=CC=CC3=CC(c4nc(-c5ccc6ccccc6c5)nc(-c5cccc6c(-c7ccc8ccccc8c7)cccc56)n4)=CC(=C1)C23 ; c1ccc2cc(-c3nc(-c4ccc5cc6ccccc6cc5c4)nc(-c4cccc5c(-c6ccc7ccccc7c6)cccc45)n3)ccc2c1 ; c1ccc(-c2nc(-c3ccccc3-c3ccccc3)nc(-c3cccc4c(-c5ccc6ccccc6c5)cccc34)n2)cc1 ; c1ccc(-c2cccc(-c3nc(-c4ccccc4)nc(-c4cccc5c(-c6ccc7ccccc7c6)cccc45)n3)c2)cc1]

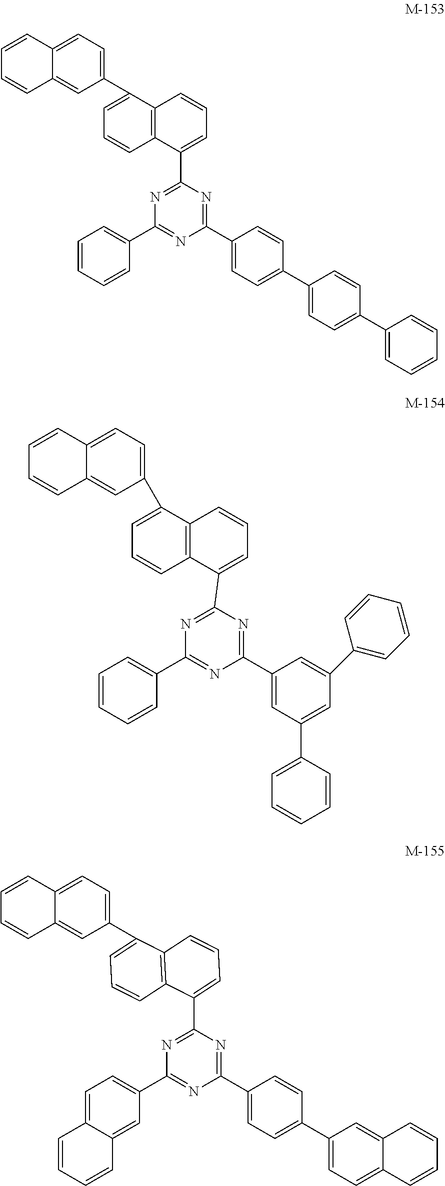 [SMILES: c1ccc(-c2ccc(-c3ccc(-c4nc(-c5ccccc5)nc(-c5cccc6c(-c7ccc8ccccc8c7)cccc56)n4)cc3)cc2)cc1 ; c1ccc(-c2cc(-c3ccccc3)cc(-c3nc(-c4ccccc4)nc(-c4cccc5c(-c6ccc7ccccc7c6)cccc45)n3)c2)cc1 ; c1ccc2cc(-c3ccc(-c4nc(-c5ccc6ccccc6c5)nc(-c5cccc6c(-c7ccc8ccccc8c7)cccc56)n4)cc3)ccc2c1]

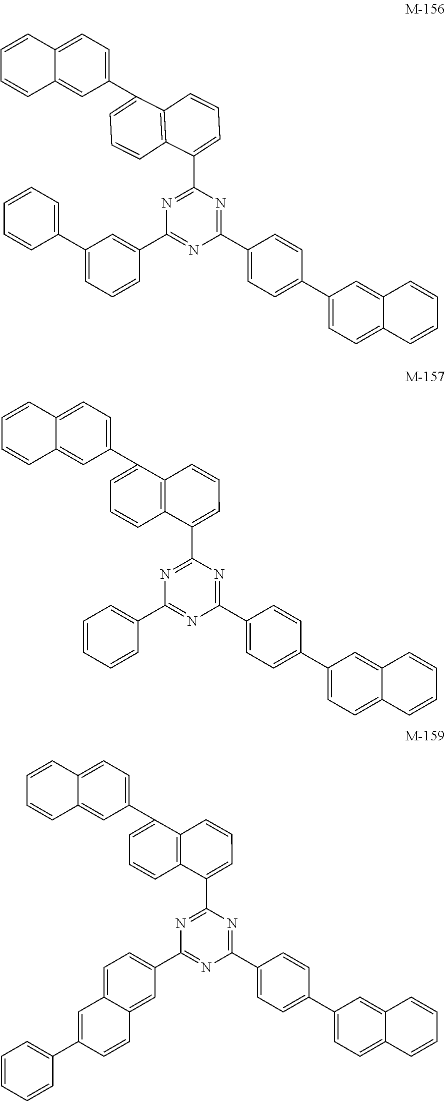 [SMILES: c1ccc(-c2cccc(-c3nc(-c4ccc(-c5ccc6ccccc6c5)cc4)nc(-c4cccc5c(-c6ccc7ccccc7c6)cccc45)n3)c2)cc1 ; c1ccc(-c2nc(-c3ccc(-c4ccc5ccccc5c4)cc3)nc(-c3cccc4c(-c5ccc6ccccc6c5)cccc34)n2)cc1 ; c1ccc(-c2ccc3cc(-c4nc(-c5ccc(-c6ccc7ccccc7c6)cc5)nc(-c5cccc6c(-c7ccc8ccccc8c7)cccc56)n4)ccc3c2)cc1]

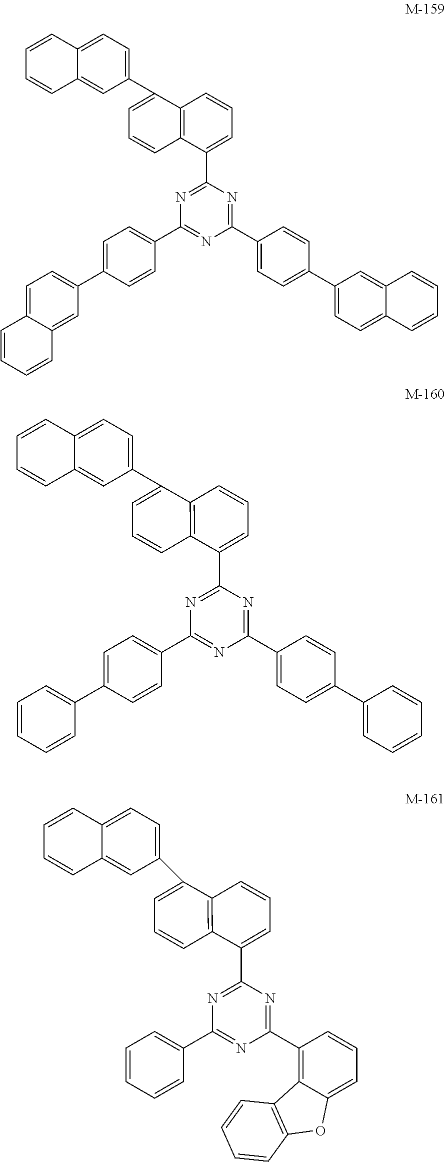 [SMILES: c1ccc2cc(-c3ccc(-c4nc(-c5ccc(-c6ccc7ccccc7c6)cc5)nc(-c5cccc6c(-c7ccc8ccccc8c7)cccc56)n4)cc3)ccc2c1 ; c1ccc(-c2ccc(-c3nc(-c4ccc(-c5ccccc5)cc4)nc(-c4cccc5c(-c6ccc7ccccc7c6)cccc45)n3)cc2)cc1 ; c1ccc(-c2nc(-c3cccc4c(-c5ccc6ccccc6c5)cccc34)nc(-c3cccc4oc5ccccc5c34)n2)cc1]

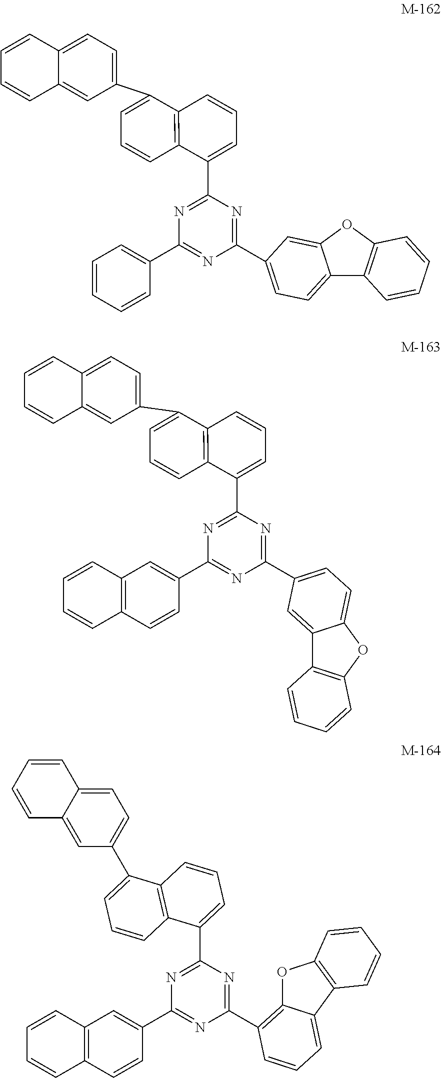

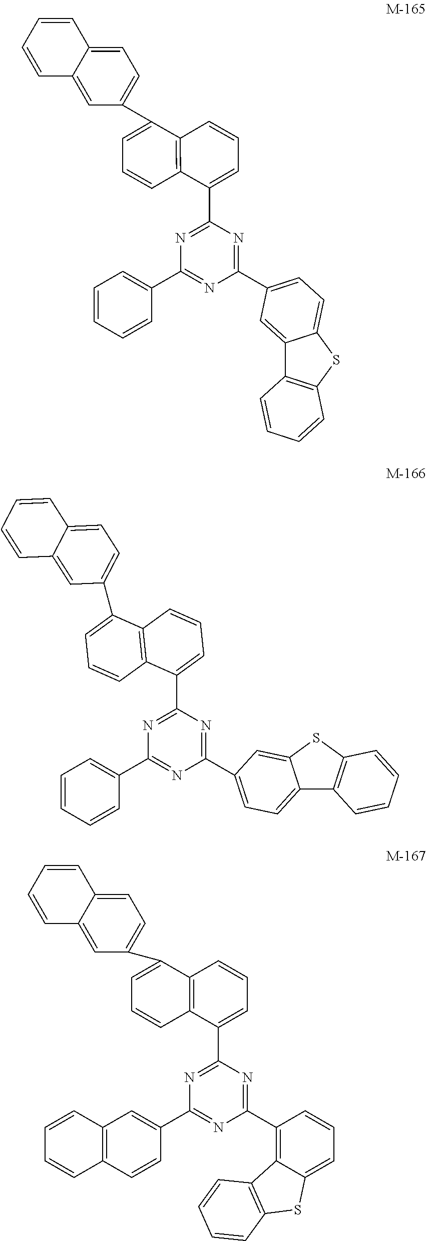

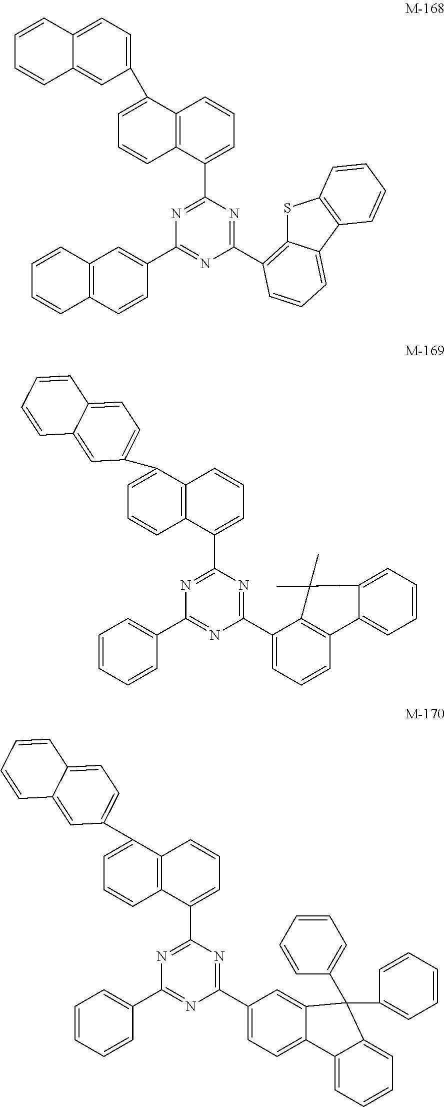 [SMILES: c1ccc2cc(-c3nc(-c4cccc5c(-c6ccc7ccccc7c6)cccc45)nc(-c4cccc5c4sc4ccccc45)n3)ccc2c1 ; CC1(C)c2ccccc2-c2cccc(-c3nc(-c4ccccc4)nc(-c4cccc5c(-c6ccc7ccccc7c6)cccc45)n3)c21 ; c1ccc(-c2nc(-c3ccc4c(c3)C(c3ccccc3)(c3ccccc3)c3ccccc3-4)nc(-c3cccc4c(-c5ccc6ccccc6c5)cccc34)n2)cc1]

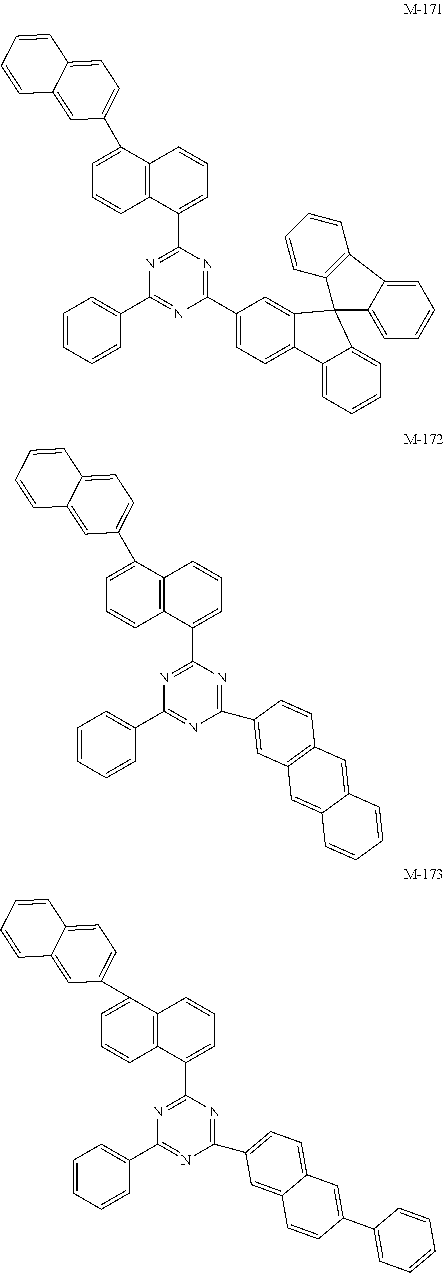 [SMILES: c1ccc(-c2nc(-c3ccc4c(c3)C3(c5ccccc5-c5ccccc53)c3ccccc3-4)nc(-c3cccc4c(-c5ccc6ccccc6c5)cccc34)n2)cc1 ; c1ccc(-c2nc(-c3ccc4cc5ccccc5cc4c3)nc(-c3cccc4c(-c5ccc6ccccc6c5)cccc34)n2)cc1 ; c1ccc(-c2ccc3cc(-c4nc(-c5ccccc5)nc(-c5cccc6c(-c7ccc8ccccc8c7)cccc56)n4)ccc3c2)cc1]

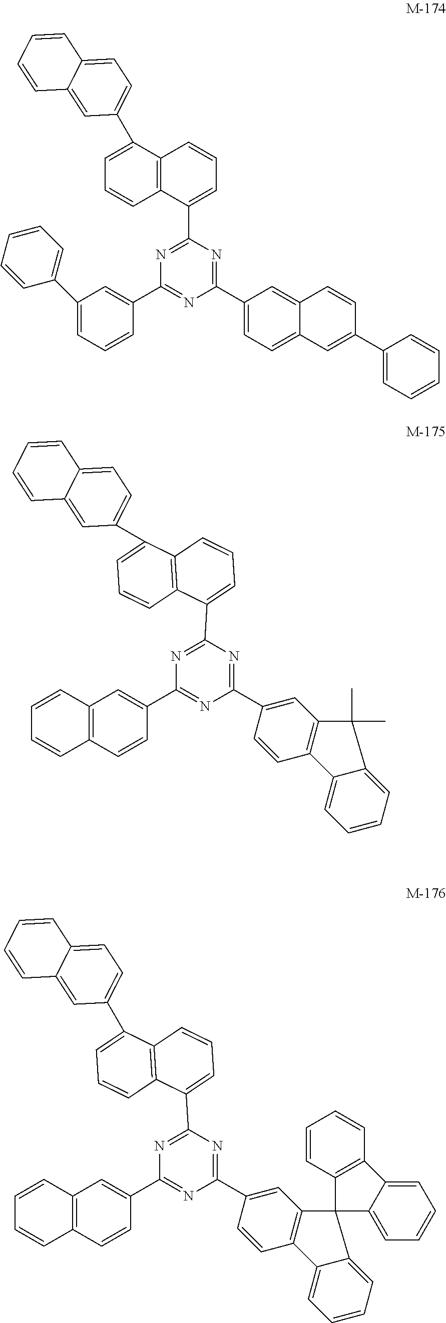 [SMILES: c1ccc(-c2cccc(-c3nc(-c4ccc5cc(-c6ccccc6)ccc5c4)nc(-c4cccc5c(-c6ccc7ccccc7c6)cccc45)n3)c2)cc1 ; CC1(C)c2ccccc2-c2ccc(-c3nc(-c4ccc5ccccc5c4)nc(-c4cccc5c(-c6ccc7ccccc7c6)cccc45)n3)cc21 ; c1ccc2c(c1)-c1ccccc1C21c2ccccc2-c2ccc(-c3nc(-c4ccc5ccccc5c4)nc(-c4cccc5c(-c6ccc7ccccc7c6)cccc45)n3)cc21]

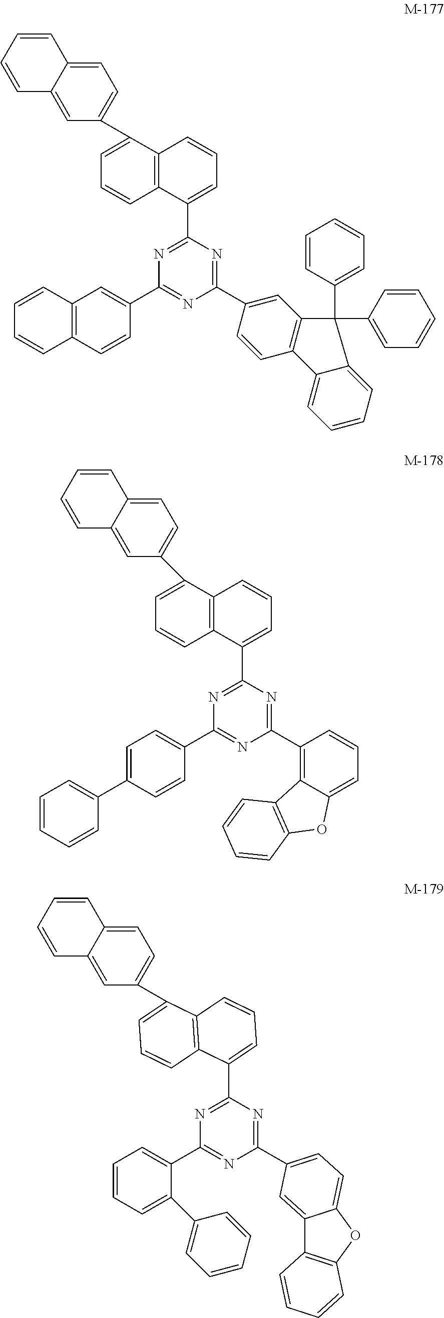 [SMILES: c1ccc(C2(c3ccccc3)c3ccccc3-c3ccc(-c4nc(-c5ccc6ccccc6c5)nc(-c5cccc6c(-c7ccc8ccccc8c7)cccc56)n4)cc32)cc1 ; c1ccc(-c2ccc(-c3nc(-c4cccc5c(-c6ccc7ccccc7c6)cccc45)nc(-c4cccc5oc6ccccc6c45)n3)cc2)cc1 ; c1ccc(-c2ccccc2-c2nc(-c3ccc4oc5ccccc5c4c3)nc(-c3cccc4c(-c5ccc6ccccc6c5)cccc34)n2)cc1]

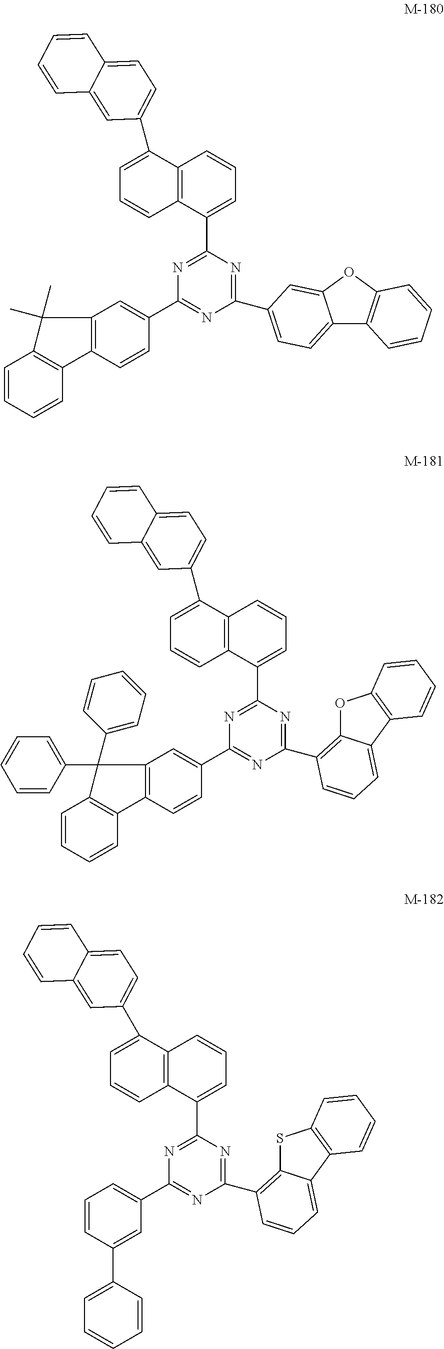 [SMILES: CC1(C)c2ccccc2-c2ccc(-c3nc(-c4ccc5c(c4)oc4ccccc45)nc(-c4cccc5c(-c6ccc7ccccc7c6)cccc45)n3)cc21 ; c1ccc(C2(c3ccccc3)c3ccccc3-c3ccc(-c4nc(-c5cccc6c(-c7ccc8ccccc8c7)cccc56)nc(-c5cccc6c5oc5ccccc56)n4)cc32)cc1 ; c1ccc(-c2cccc(-c3nc(-c4cccc5c(-c6ccc7ccccc7c6)cccc45)nc(-c4cccc5c4sc4ccccc45)n3)c2)cc1]

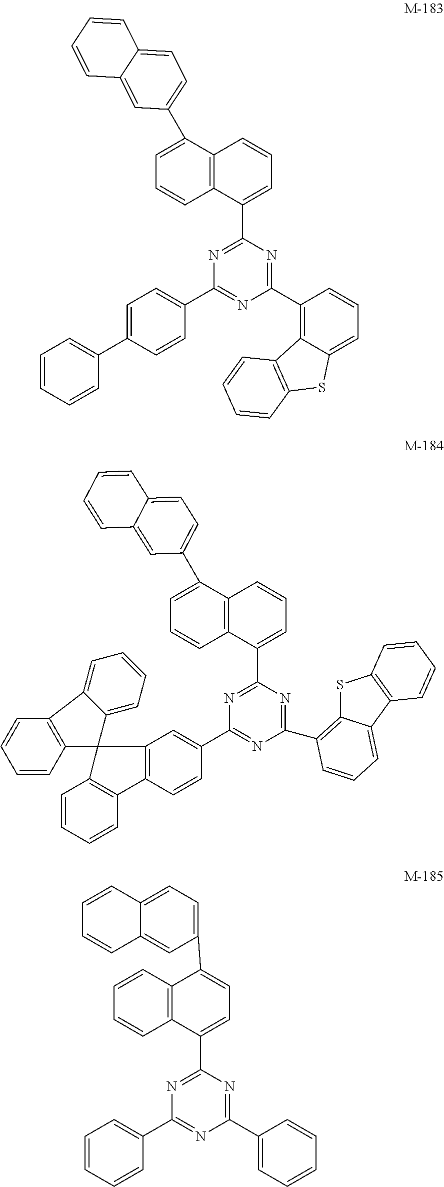 [SMILES: c1ccc(-c2ccc(-c3nc(-c4cccc5c(-c6ccc7ccccc7c6)cccc45)nc(-c4cccc5sc6ccccc6c45)n3)cc2)cc1 ; c1ccc2c(c1)-c1ccccc1C21c2ccccc2-c2ccc(-c3nc(-c4cccc5c(-c6ccc7ccccc7c6)cccc45)nc(-c4cccc5c4sc4ccccc45)n3)cc21 ; c1ccc(-c2nc(-c3ccccc3)nc(-c3ccc(-c4ccc5ccccc5c4)c4ccccc34)n2)cc1]

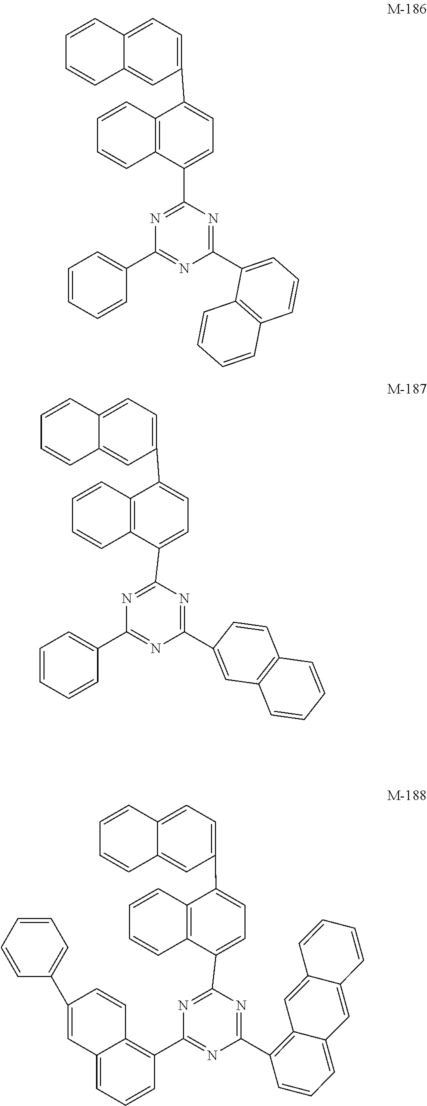 [SMILES: c1ccc(-c2nc(-c3cccc4ccccc34)nc(-c3ccc(-c4ccc5ccccc5c4)c4ccccc34)n2)cc1 ; c1ccc(-c2nc(-c3ccc4ccccc4c3)nc(-c3ccc(-c4ccc5ccccc5c4)c4ccccc34)n2)cc1 ; c1ccc(-c2ccc3c(-c4nc(-c5cccc6cc7ccccc7cc56)nc(-c5ccc(-c6ccc7ccccc7c6)c6ccccc56)n4)cccc3c2)cc1]

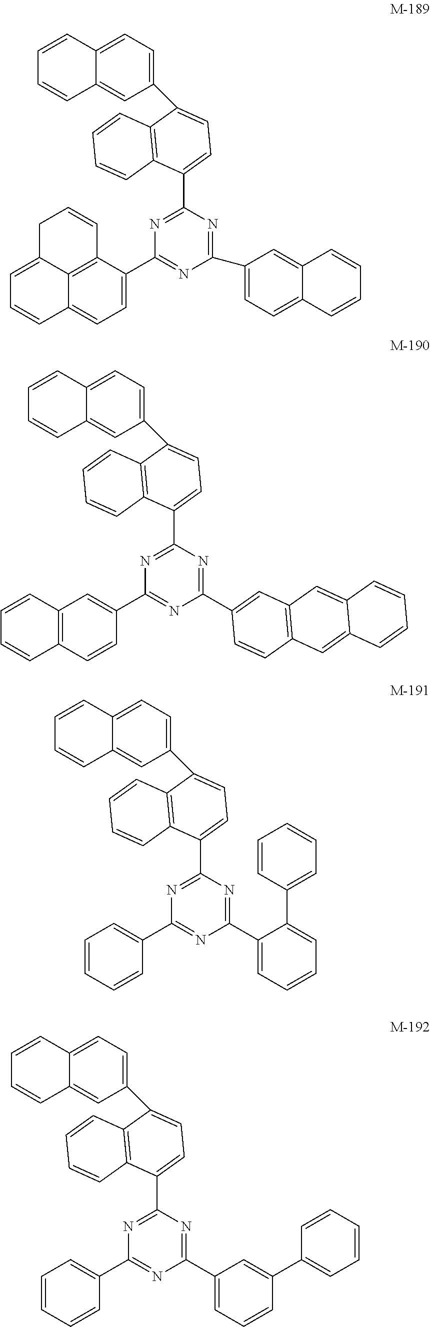

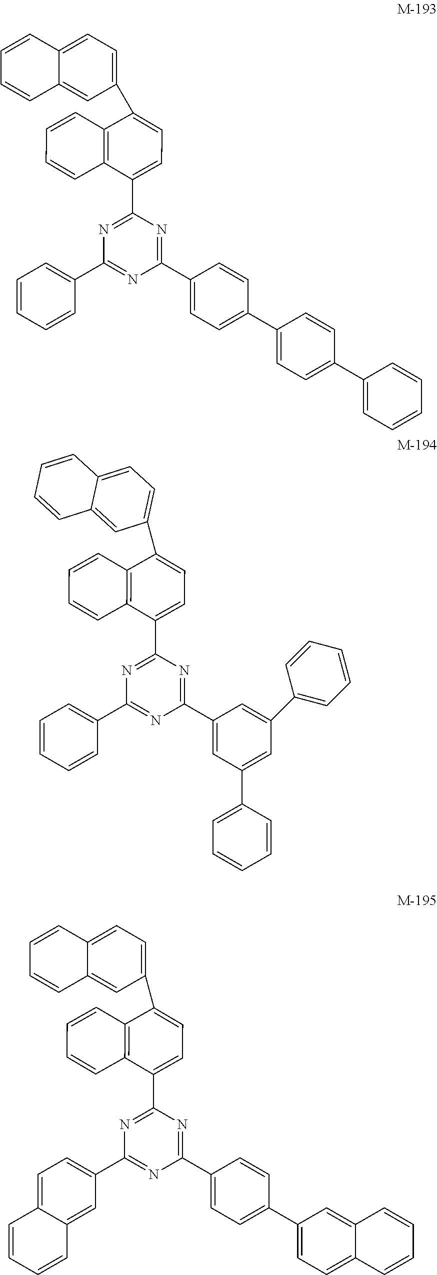 [SMILES: c1ccc(-c2ccc(-c3ccc(-c4nc(-c5ccccc5)nc(-c5ccc(-c6ccc7ccccc7c6)c6ccccc56)n4)cc3)cc2)cc1 ; c1ccc(-c2cc(-c3ccccc3)cc(-c3nc(-c4ccccc4)nc(-c4ccc(-c5ccc6ccccc6c5)c5ccccc45)n3)c2)cc1 ; c1ccc2cc(-c3ccc(-c4nc(-c5ccc6ccccc6c5)nc(-c5ccc(-c6ccc7ccccc7c6)c6ccccc56)n4)cc3)ccc2c1]

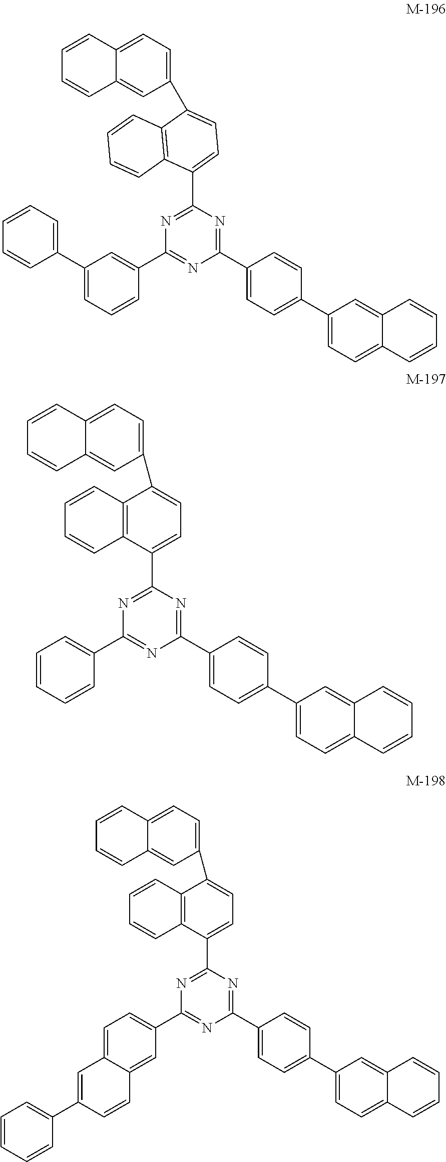 [SMILES: c1ccc(-c2cccc(-c3nc(-c4ccc(-c5ccc6ccccc6c5)cc4)nc(-c4ccc(-c5ccc6ccccc6c5)c5ccccc45)n3)c2)cc1 ; c1ccc(-c2nc(-c3ccc(-c4ccc5ccccc5c4)cc3)nc(-c3ccc(-c4ccc5ccccc5c4)c4ccccc34)n2)cc1 ; c1ccc(-c2ccc3cc(-c4nc(-c5ccc(-c6ccc7ccccc7c6)cc5)nc(-c5ccc(-c6ccc7ccccc7c6)c6ccccc56)n4)ccc3c2)cc1]

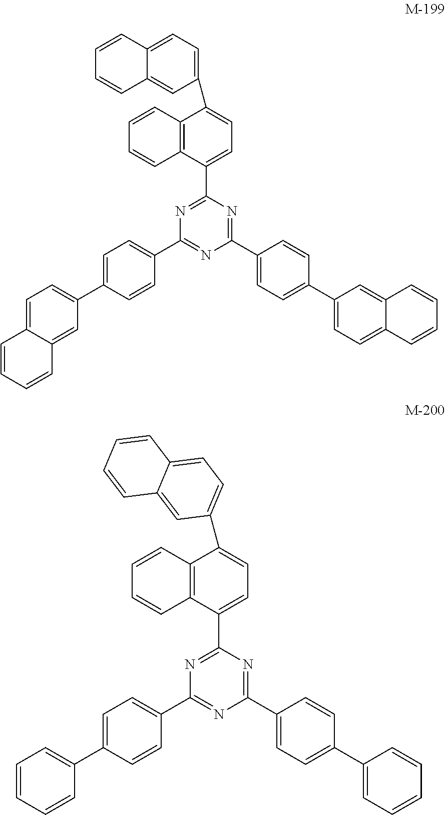 [SMILES: c1ccc2cc(-c3ccc(-c4nc(-c5ccc(-c6ccc7ccccc7c6)cc5)nc(-c5ccc(-c6ccc7ccccc7c6)c6ccccc56)n4)cc3)ccc2c1 ; c1ccc(-c2ccc(-c3nc(-c4ccc(-c5ccccc5)cc4)nc(-c4ccc(-c5ccc6ccccc6c5)c5ccccc45)n3)cc2)cc1]

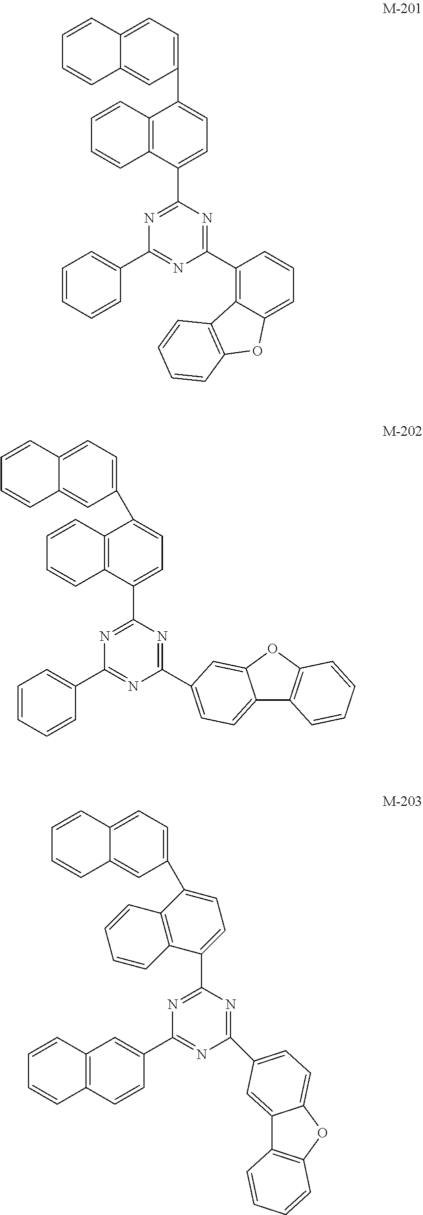 [SMILES: c1ccc(-c2nc(-c3ccc(-c4ccc5ccccc5c4)c4ccccc34)nc(-c3cccc4oc5ccccc5c34)n2)cc1 ; c1ccc(-c2nc(-c3ccc4c(c3)oc3ccccc34)nc(-c3ccc(-c4ccc5ccccc5c4)c4ccccc34)n2)cc1 ; c1ccc2cc(-c3nc(-c4ccc5oc6ccccc6c5c4)nc(-c4ccc(-c5ccc6ccccc6c5)c5ccccc45)n3)ccc2c1]

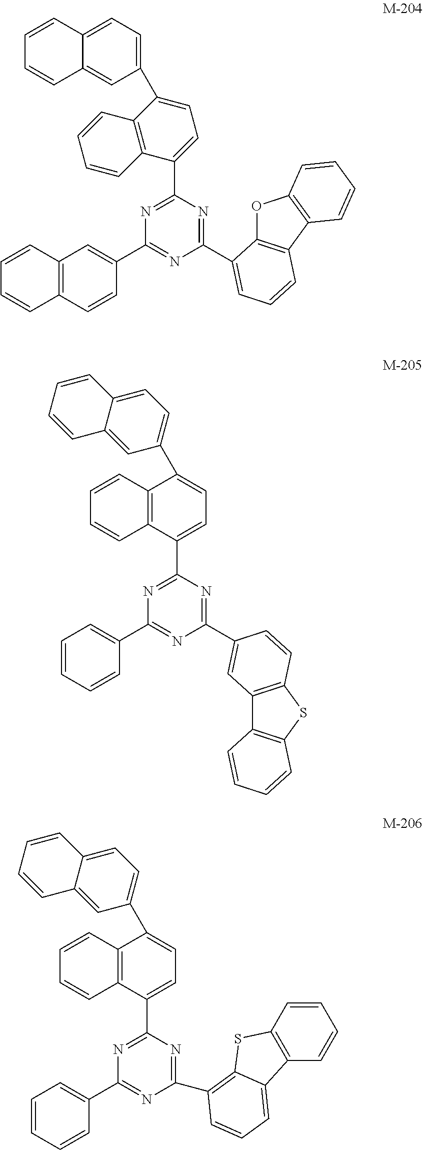 [SMILES: c1ccc2cc(-c3nc(-c4ccc(-c5ccc6ccccc6c5)c5ccccc45)nc(-c4cccc5c4oc4ccccc45)n3)ccc2c1 ; c1ccc(-c2nc(-c3ccc4sc5ccccc5c4c3)nc(-c3ccc(-c4ccc5ccccc5c4)c4ccccc34)n2)cc1 ; c1ccc(-c2nc(-c3ccc(-c4ccc5ccccc5c4)c4ccccc34)nc(-c3cccc4c3sc3ccccc34)n2)cc1]

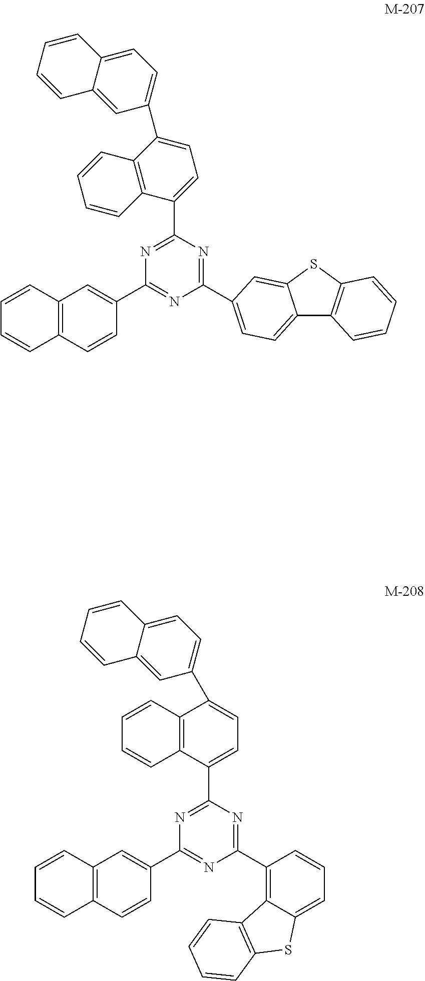 [SMILES: c1ccc2cc(-c3nc(-c4ccc5c(c4)sc4ccccc45)nc(-c4ccc(-c5ccc6ccccc6c5)c5ccccc45)n3)ccc2c1 ; c1ccc2cc(-c3nc(-c4ccc(-c5ccc6ccccc6c5)c5ccccc45)nc(-c4cccc5sc6ccccc6c45)n3)ccc2c1]

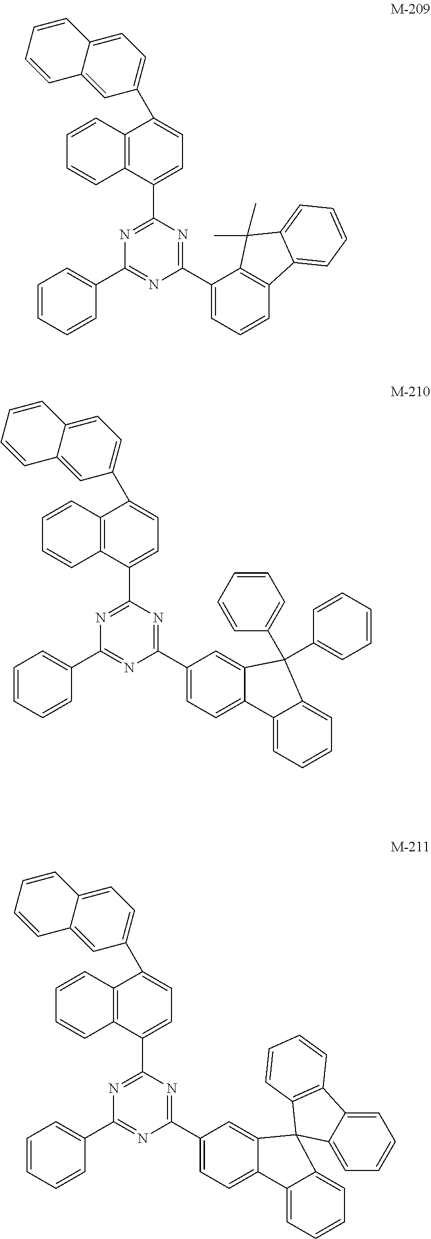 [SMILES: CC1(C)c2ccccc2-c2cccc(-c3nc(-c4ccccc4)nc(-c4ccc(-c5ccc6ccccc6c5)c5ccccc45)n3)c21 ; c1ccc(-c2nc(-c3ccc4c(c3)C(c3ccccc3)(c3ccccc3)c3ccccc3-4)nc(-c3ccc(-c4ccc5ccccc5c4)c4ccccc34)n2)cc1 ; c1ccc(-c2nc(-c3ccc4c(c3)C3(c5ccccc5-c5ccccc53)c3ccccc3-4)nc(-c3ccc(-c4ccc5ccccc5c4)c4ccccc34)n2)cc1]

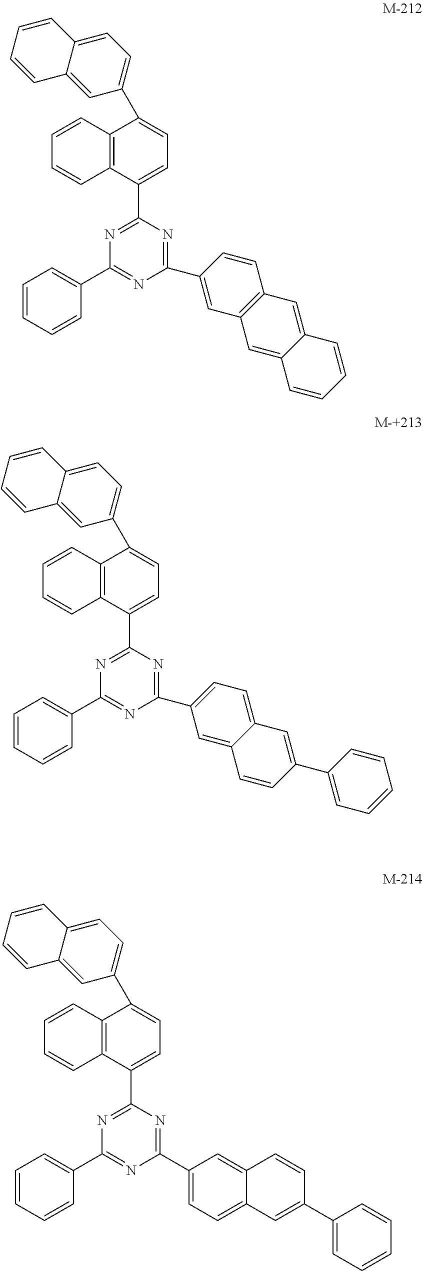 [SMILES: c1ccc(-c2nc(-c3ccc4cc5ccccc5cc4c3)nc(-c3ccc(-c4ccc5ccccc5c4)c4ccccc34)n2)cc1 ; c1ccc(-c2ccc3cc(-c4nc(-c5ccccc5)nc(-c5ccc(-c6ccc7ccccc7c6)c6ccccc56)n4)ccc3c2)cc1 ; c1ccc(-c2ccc3cc(-c4nc(-c5ccccc5)nc(-c5ccc(-c6ccc7ccccc7c6)c6ccccc56)n4)ccc3c2)cc1]

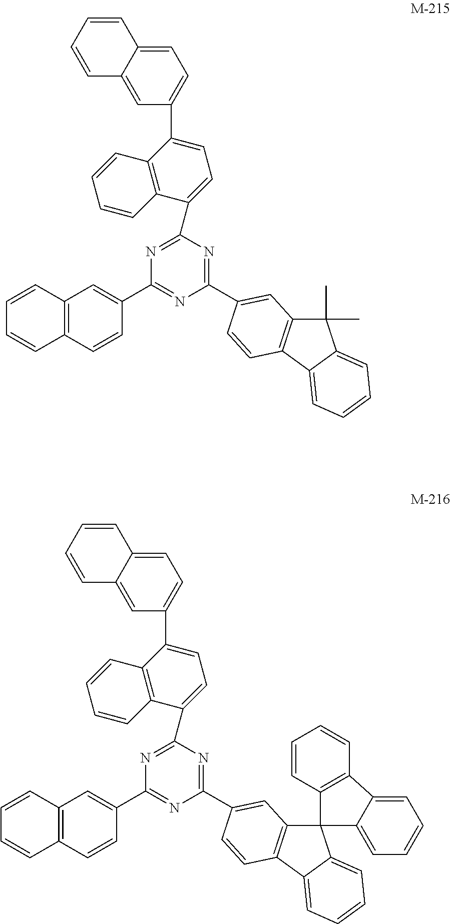 [SMILES: CC1(C)c2ccccc2-c2ccc(-c3nc(-c4ccc5ccccc5c4)nc(-c4ccc(-c5ccc6ccccc6c5)c5ccccc45)n3)cc21 ; c1ccc2c(c1)-c1ccccc1C21c2ccccc2-c2ccc(-c3nc(-c4ccc5ccccc5c4)nc(-c4ccc(-c5ccc6ccccc6c5)c5ccccc45)n3)cc21]

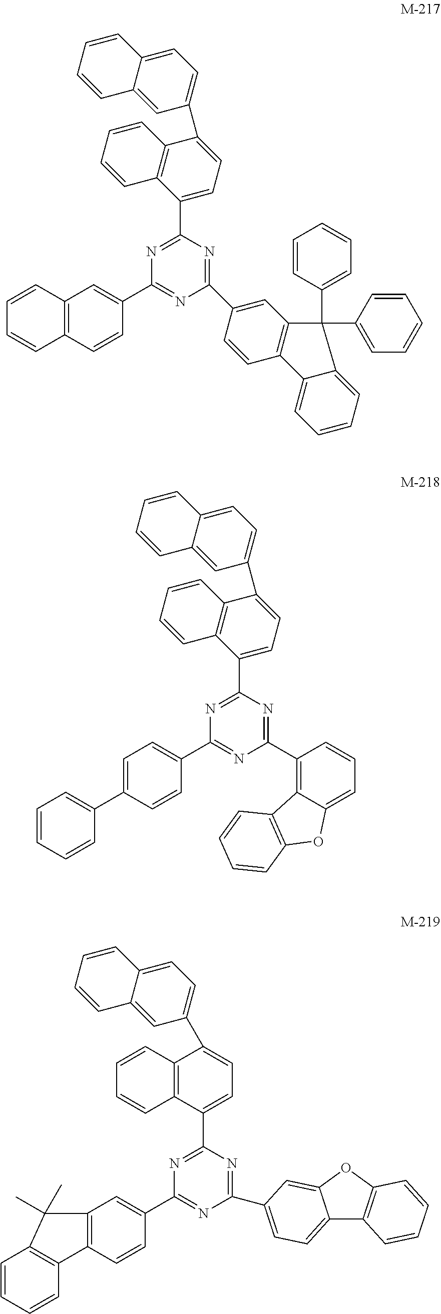 [SMILES: c1ccc(C2(c3ccccc3)c3ccccc3-c3ccc(-c4nc(-c5ccc6ccccc6c5)nc(-c5ccc(-c6ccc7ccccc7c6)c6ccccc56)n4)cc32)cc1 ; c1ccc(-c2ccc(-c3nc(-c4ccc(-c5ccc6ccccc6c5)c5ccccc45)nc(-c4cccc5oc6ccccc6c45)n3)cc2)cc1 ; CC1(C)c2ccccc2-c2ccc(-c3nc(-c4ccc5c(c4)oc4ccccc45)nc(-c4ccc(-c5ccc6ccccc6c5)c5ccccc45)n3)cc21]

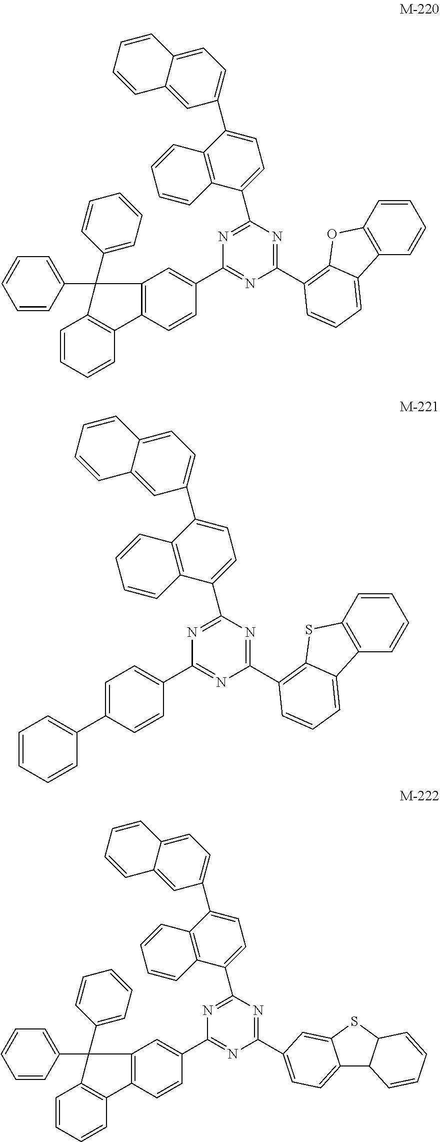 [SMILES: c1ccc(C2(c3ccccc3)c3ccccc3-c3ccc(-c4nc(-c5ccc(-c6ccc7ccccc7c6)c6ccccc56)nc(-c5cccc6c5oc5ccccc56)n4)cc32)cc1 ; c1ccc(-c2ccc(-c3nc(-c4ccc(-c5ccc6ccccc6c5)c5ccccc45)nc(-c4cccc5c4sc4ccccc45)n3)cc2)cc1 ; C1=CC2Sc3cc(-c4nc(-c5ccc6c(c5)C(c5ccccc5)(c5ccccc5)c5ccccc5-6)nc(-c5ccc(-c6ccc7ccccc7c6)c6ccccc56)n4)ccc3C2C=C1]

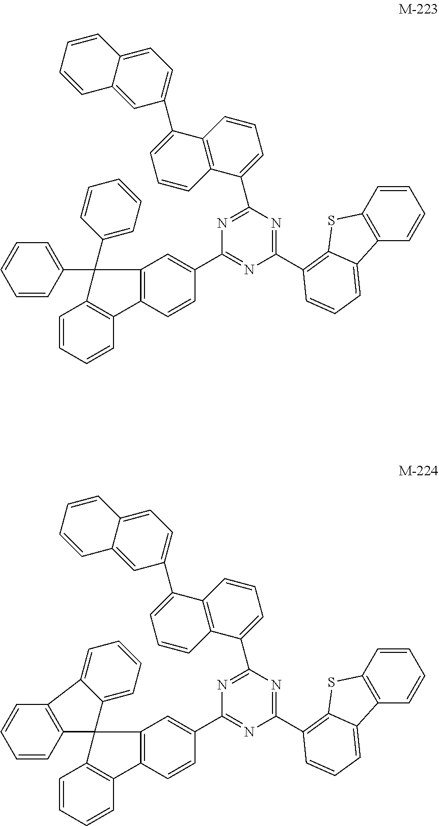 [SMILES: c1ccc(C2(c3ccccc3)c3ccccc3-c3ccc(-c4nc(-c5cccc6c(-c7ccc8ccccc8c7)cccc56)nc(-c5cccc6c5sc5ccccc56)n4)cc32)cc1 ; c1ccc2c(c1)-c1ccccc1C21c2ccccc2-c2ccc(-c3nc(-c4cccc5c(-c6ccc7ccccc7c6)cccc45)nc(-c4cccc5c4sc4ccccc45)n3)cc21]

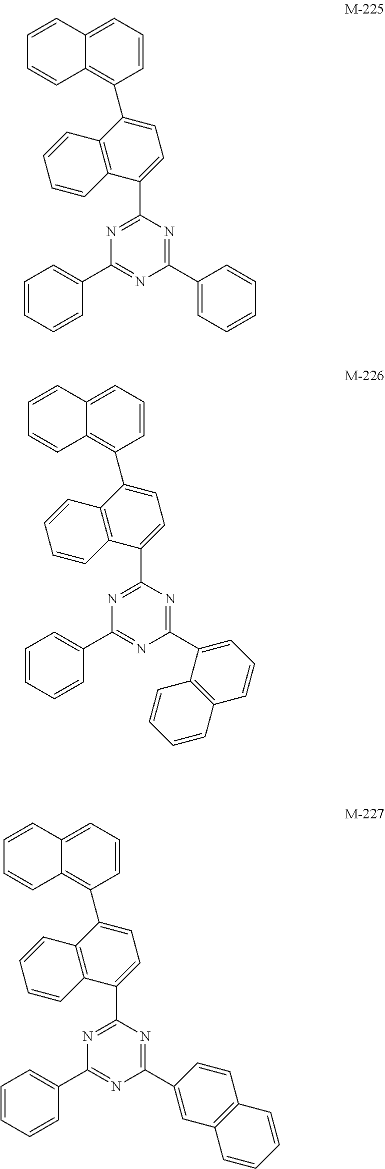 [SMILES: c1ccc(-c2nc(-c3ccccc3)nc(-c3ccc(-c4cccc5ccccc45)c4ccccc34)n2)cc1 ; c1ccc(-c2nc(-c3cccc4ccccc34)nc(-c3ccc(-c4cccc5ccccc45)c4ccccc34)n2)cc1 ; c1ccc(-c2nc(-c3ccc4ccccc4c3)nc(-c3ccc(-c4cccc5ccccc45)c4ccccc34)n2)cc1]

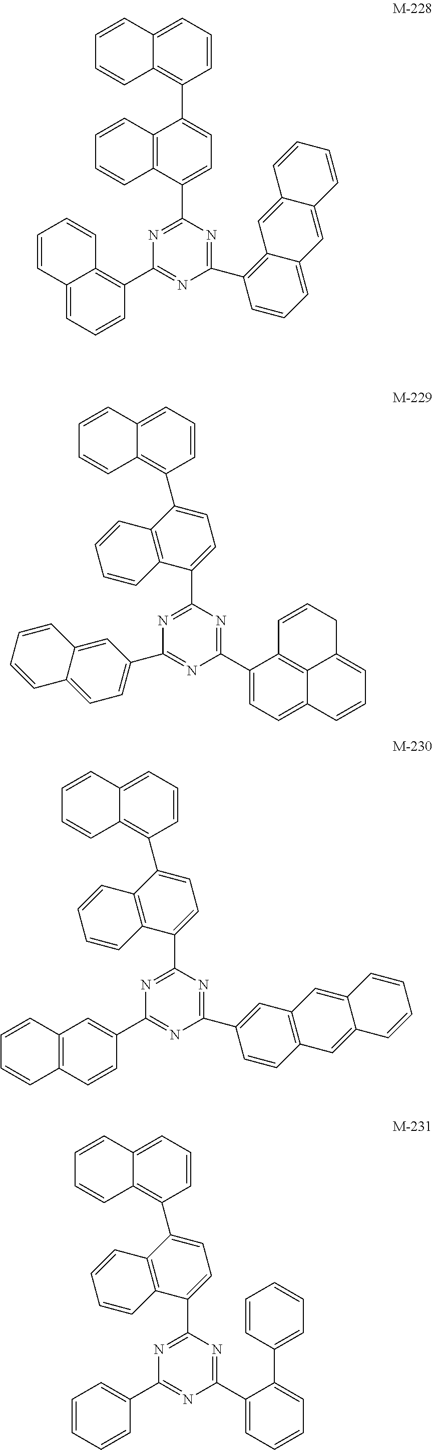 [SMILES: c1ccc2cc3c(-c4nc(-c5cccc6ccccc56)nc(-c5ccc(-c6cccc7ccccc67)c6ccccc56)n4)cccc3cc2c1 ; C1=Cc2c(-c3nc(-c4ccc5ccccc5c4)nc(-c4ccc(-c5cccc6ccccc56)c5ccccc45)n3)ccc3cccc(c23)C1 ; c1ccc2cc(-c3nc(-c4ccc5cc6ccccc6cc5c4)nc(-c4ccc(-c5cccc6ccccc56)c5ccccc45)n3)ccc2c1 ; c1ccc(-c2nc(-c3ccccc3-c3ccccc3)nc(-c3ccc(-c4cccc5ccccc45)c4ccccc34)n2)cc1]

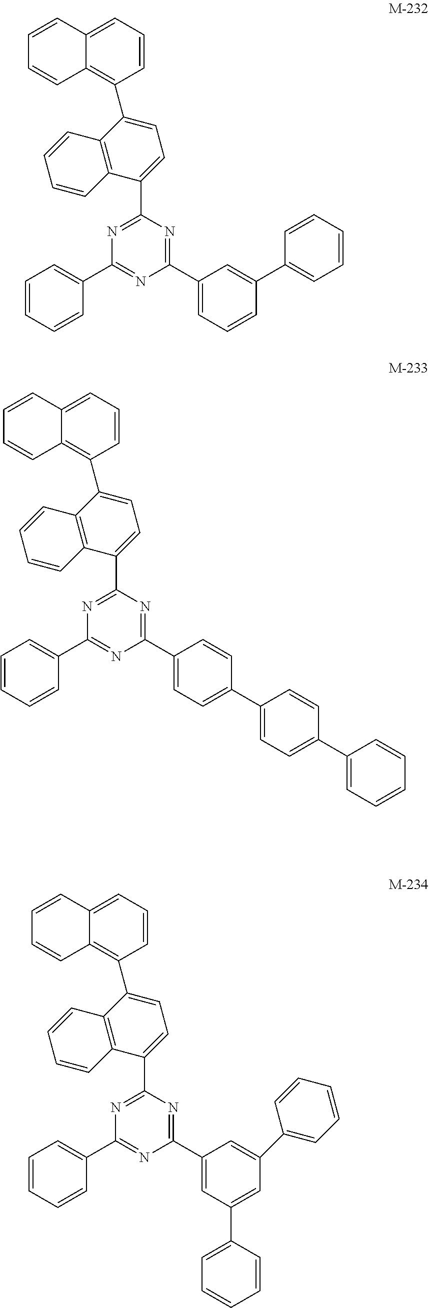 [SMILES: c1ccc(-c2cccc(-c3nc(-c4ccccc4)nc(-c4ccc(-c5cccc6ccccc56)c5ccccc45)n3)c2)cc1 ; c1ccc(-c2ccc(-c3ccc(-c4nc(-c5ccccc5)nc(-c5ccc(-c6cccc7ccccc67)c6ccccc56)n4)cc3)cc2)cc1 ; c1ccc(-c2cc(-c3ccccc3)cc(-c3nc(-c4ccccc4)nc(-c4ccc(-c5cccc6ccccc56)c5ccccc45)n3)c2)cc1]

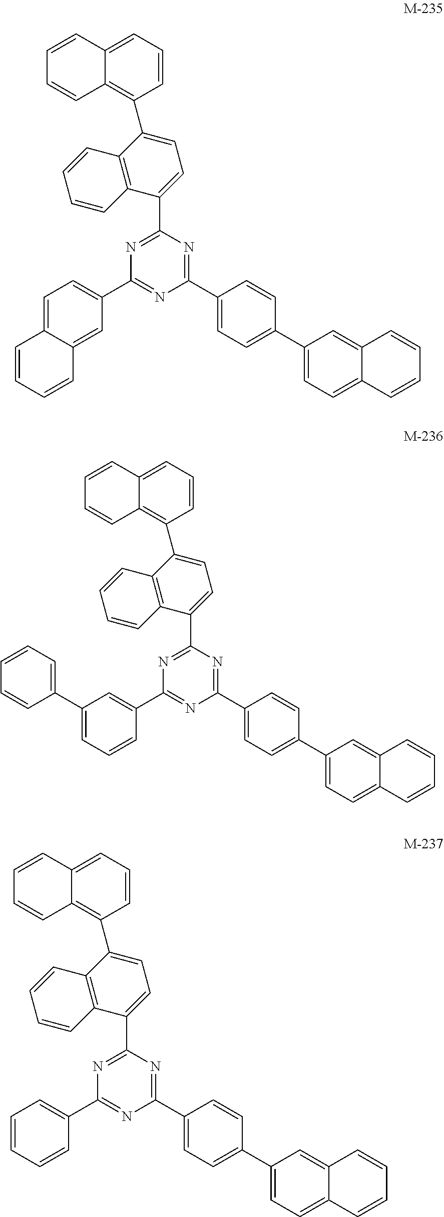 [SMILES: c1ccc2cc(-c3ccc(-c4nc(-c5ccc6ccccc6c5)nc(-c5ccc(-c6cccc7ccccc67)c6ccccc56)n4)cc3)ccc2c1 ; c1ccc(-c2cccc(-c3nc(-c4ccc(-c5ccc6ccccc6c5)cc4)nc(-c4ccc(-c5cccc6ccccc56)c5ccccc45)n3)c2)cc1 ; c1ccc(-c2nc(-c3ccc(-c4ccc5ccccc5c4)cc3)nc(-c3ccc(-c4cccc5ccccc45)c4ccccc34)n2)cc1]

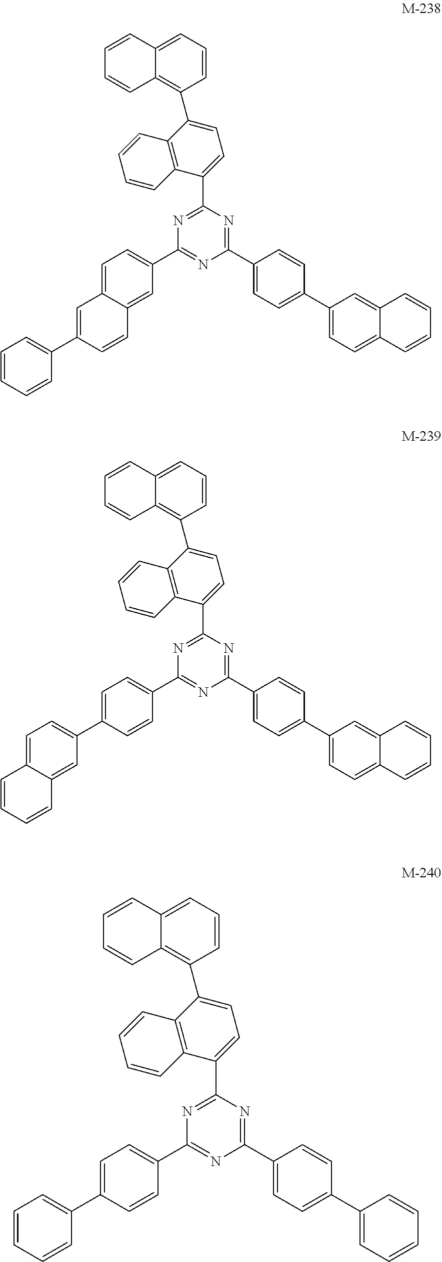 [SMILES: c1ccc(-c2ccc3cc(-c4nc(-c5ccc(-c6ccc7ccccc7c6)cc5)nc(-c5ccc(-c6cccc7ccccc67)c6ccccc56)n4)ccc3c2)cc1 ; c1ccc2cc(-c3ccc(-c4nc(-c5ccc(-c6ccc7ccccc7c6)cc5)nc(-c5ccc(-c6cccc7ccccc67)c6ccccc56)n4)cc3)ccc2c1 ; c1ccc(-c2ccc(-c3nc(-c4ccc(-c5ccccc5)cc4)nc(-c4ccc(-c5cccc6ccccc56)c5ccccc45)n3)cc2)cc1]

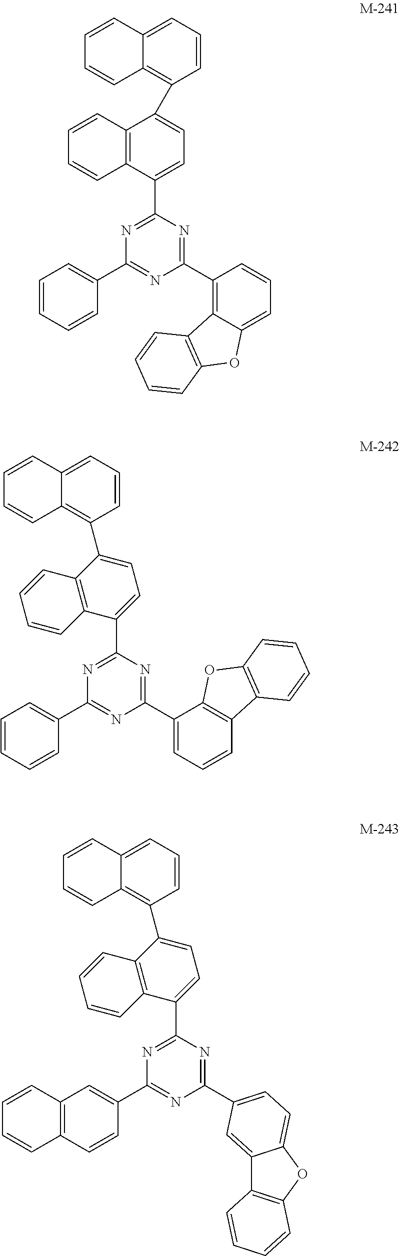 [SMILES: c1ccc(-c2nc(-c3ccc(-c4cccc5ccccc45)c4ccccc34)nc(-c3cccc4oc5ccccc5c34)n2)cc1 ; c1ccc(-c2nc(-c3ccc(-c4cccc5ccccc45)c4ccccc34)nc(-c3cccc4c3oc3ccccc34)n2)cc1 ; c1ccc2cc(-c3nc(-c4ccc5oc6ccccc6c5c4)nc(-c4ccc(-c5cccc6ccccc56)c5ccccc45)n3)ccc2c1]

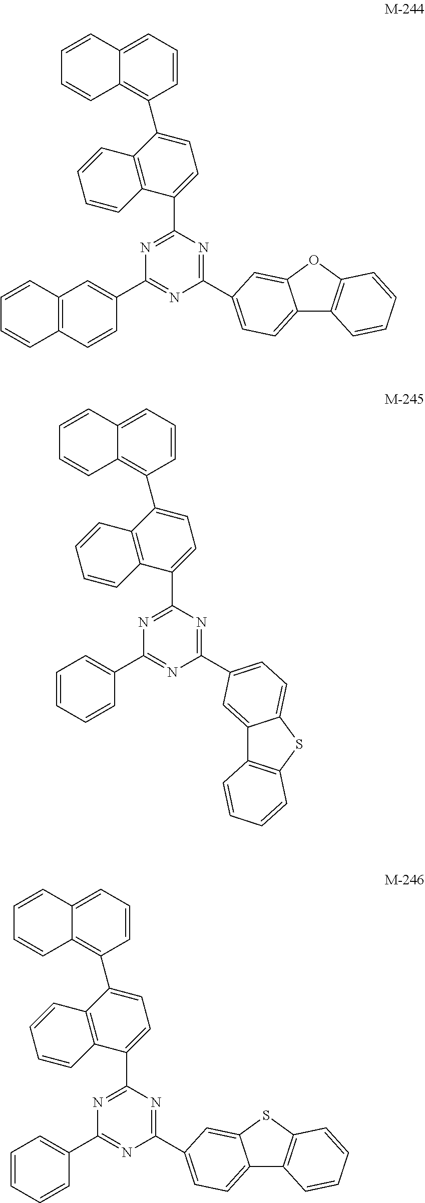 [SMILES: c1ccc2cc(-c3nc(-c4ccc5c(c4)oc4ccccc45)nc(-c4ccc(-c5cccc6ccccc56)c5ccccc45)n3)ccc2c1 ; c1ccc(-c2nc(-c3ccc4sc5ccccc5c4c3)nc(-c3ccc(-c4cccc5ccccc45)c4ccccc34)n2)cc1 ; c1ccc(-c2nc(-c3ccc4c(c3)sc3ccccc34)nc(-c3ccc(-c4cccc5ccccc45)c4ccccc34)n2)cc1]

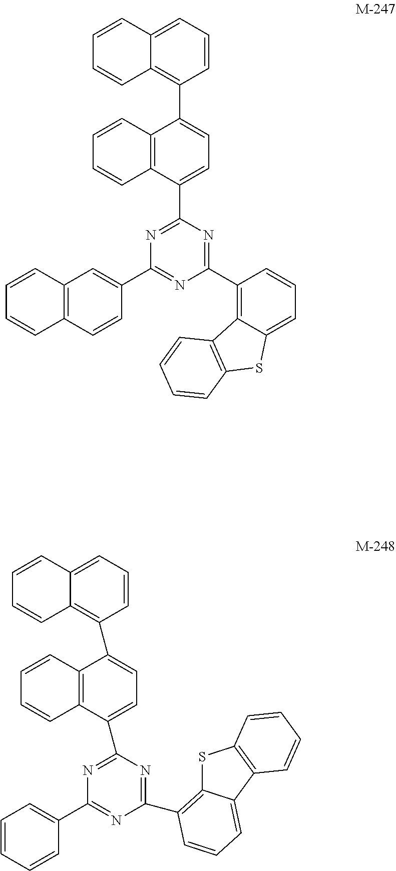 [SMILES: c1ccc2cc(-c3nc(-c4ccc(-c5cccc6ccccc56)c5ccccc45)nc(-c4cccc5sc6ccccc6c45)n3)ccc2c1 ; c1ccc(-c2nc(-c3ccc(-c4cccc5ccccc45)c4ccccc34)nc(-c3cccc4c3sc3ccccc34)n2)cc1]

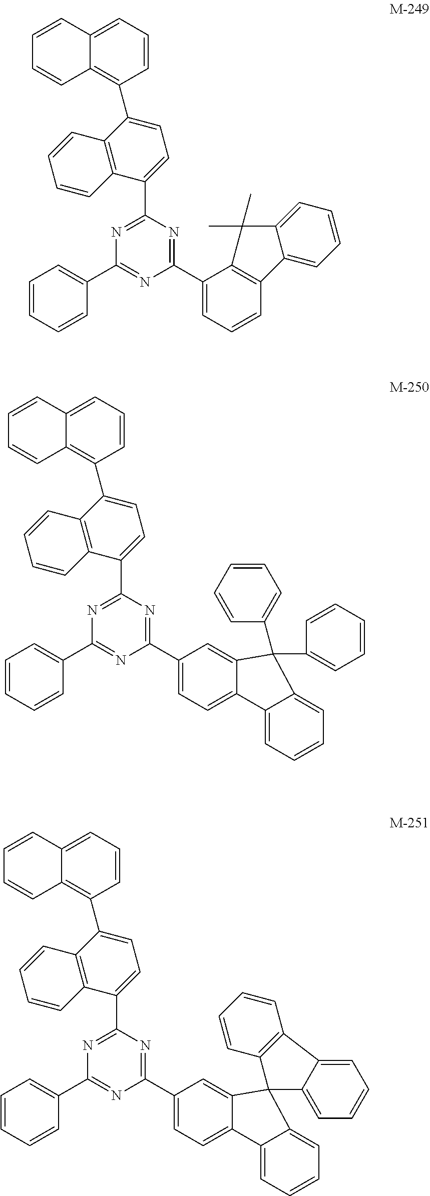 [SMILES: CC1(C)c2ccccc2-c2cccc(-c3nc(-c4ccccc4)nc(-c4ccc(-c5cccc6ccccc56)c5ccccc45)n3)c21 ; c1ccc(-c2nc(-c3ccc4c(c3)C(c3ccccc3)(c3ccccc3)c3ccccc3-4)nc(-c3ccc(-c4cccc5ccccc45)c4ccccc34)n2)cc1 ; c1ccc(-c2nc(-c3ccc4c(c3)C3(c5ccccc5-c5ccccc53)c3ccccc3-4)nc(-c3ccc(-c4cccc5ccccc45)c4ccccc34)n2)cc1]

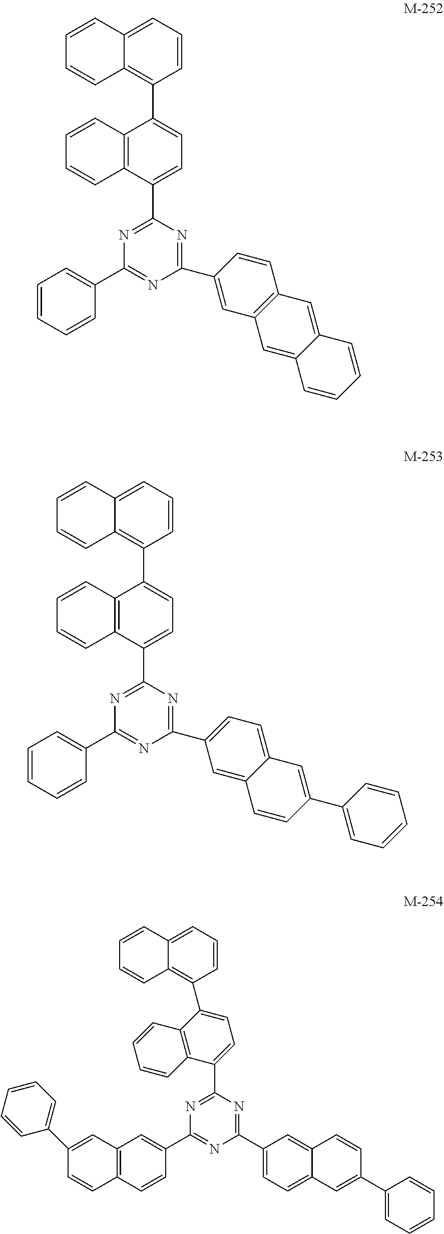 [SMILES: c1ccc(-c2nc(-c3ccc4cc5ccccc5cc4c3)nc(-c3ccc(-c4cccc5ccccc45)c4ccccc34)n2)cc1 ; c1ccc(-c2ccc3cc(-c4nc(-c5ccccc5)nc(-c5ccc(-c6cccc7ccccc67)c6ccccc56)n4)ccc3c2)cc1 ; c1ccc(-c2ccc3cc(-c4nc(-c5ccc6ccc(-c7ccccc7)cc6c5)nc(-c5ccc(-c6cccc7ccccc67)c6ccccc56)n4)ccc3c2)cc1]

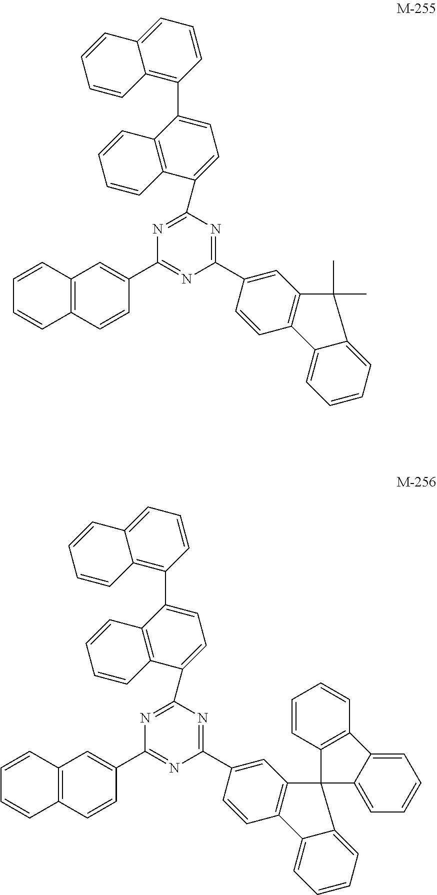 [SMILES: CC1(C)c2ccccc2-c2ccc(-c3nc(-c4ccc5ccccc5c4)nc(-c4ccc(-c5cccc6ccccc56)c5ccccc45)n3)cc21 ; c1ccc2c(c1)-c1ccccc1C21c2ccccc2-c2ccc(-c3nc(-c4ccc5ccccc5c4)nc(-c4ccc(-c5cccc6ccccc56)c5ccccc45)n3)cc21]

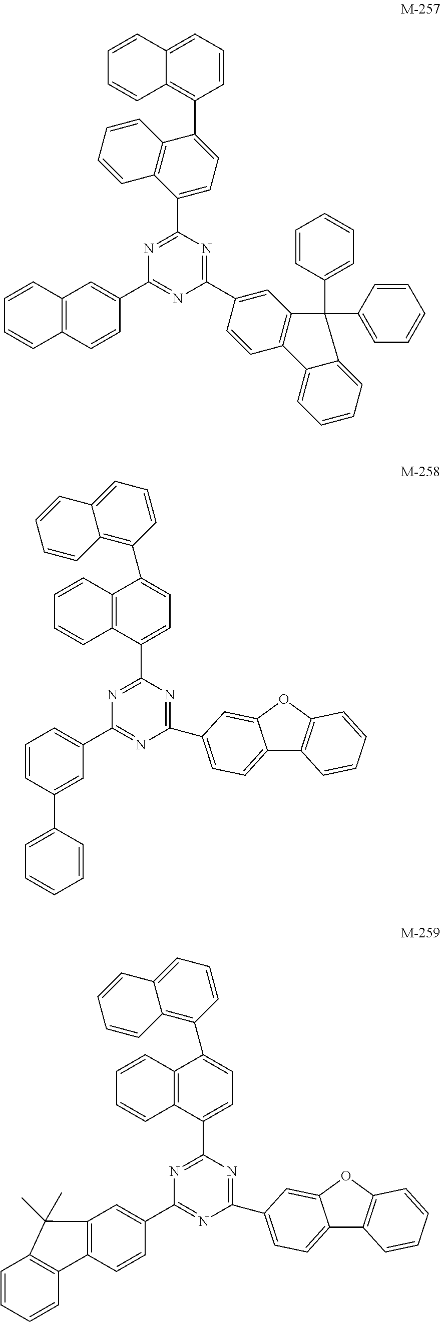 [SMILES: c1ccc(C2(c3ccccc3)c3ccccc3-c3ccc(-c4nc(-c5ccc6ccccc6c5)nc(-c5ccc(-c6cccc7ccccc67)c6ccccc56)n4)cc32)cc1 ; c1ccc(-c2cccc(-c3nc(-c4ccc5c(c4)oc4ccccc45)nc(-c4ccc(-c5cccc6ccccc56)c5ccccc45)n3)c2)cc1 ; CC1(C)c2ccccc2-c2ccc(-c3nc(-c4ccc5c(c4)oc4ccccc45)nc(-c4ccc(-c5cccc6ccccc56)c5ccccc45)n3)cc21]

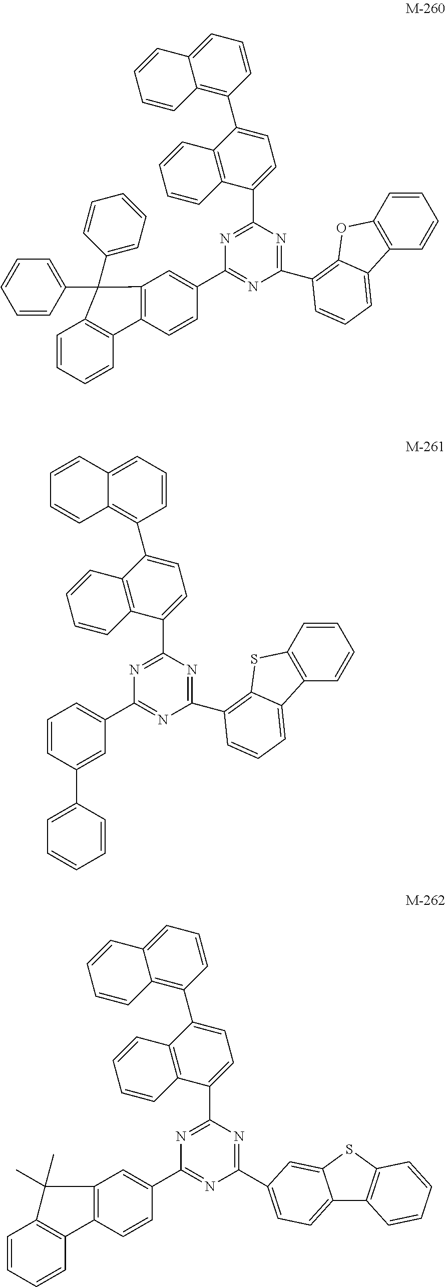 [SMILES: c1ccc(C2(c3ccccc3)c3ccccc3-c3ccc(-c4nc(-c5ccc(-c6cccc7ccccc67)c6ccccc56)nc(-c5cccc6c5oc5ccccc56)n4)cc32)cc1 ; c1ccc(-c2cccc(-c3nc(-c4ccc(-c5cccc6ccccc56)c5ccccc45)nc(-c4cccc5c4sc4ccccc45)n3)c2)cc1 ; CC1(C)c2ccccc2-c2ccc(-c3nc(-c4ccc5c(c4)sc4ccccc45)nc(-c4ccc(-c5cccc6ccccc56)c5ccccc45)n3)cc21]

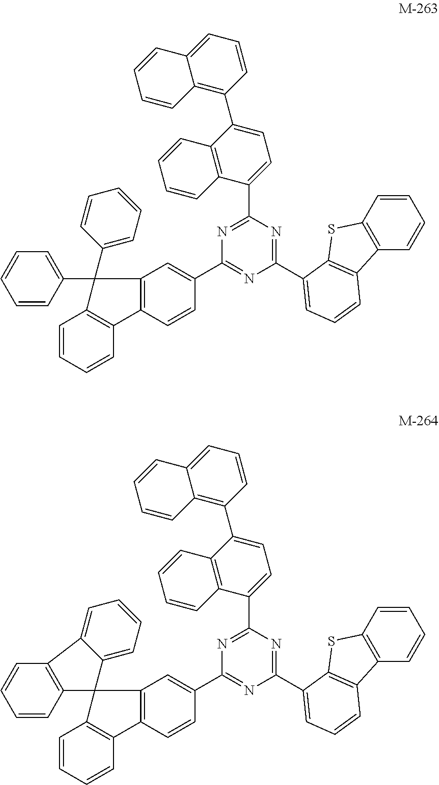 [SMILES: c1ccc(C2(c3ccccc3)c3ccccc3-c3ccc(-c4nc(-c5ccc(-c6cccc7ccccc67)c6ccccc56)nc(-c5cccc6c5sc5ccccc56)n4)cc32)cc1 ; c1ccc2c(c1)-c1ccccc1C21c2ccccc2-c2ccc(-c3nc(-c4ccc(-c5cccc6ccccc56)c5ccccc45)nc(-c4cccc5c4sc4ccccc45)n3)cc21]

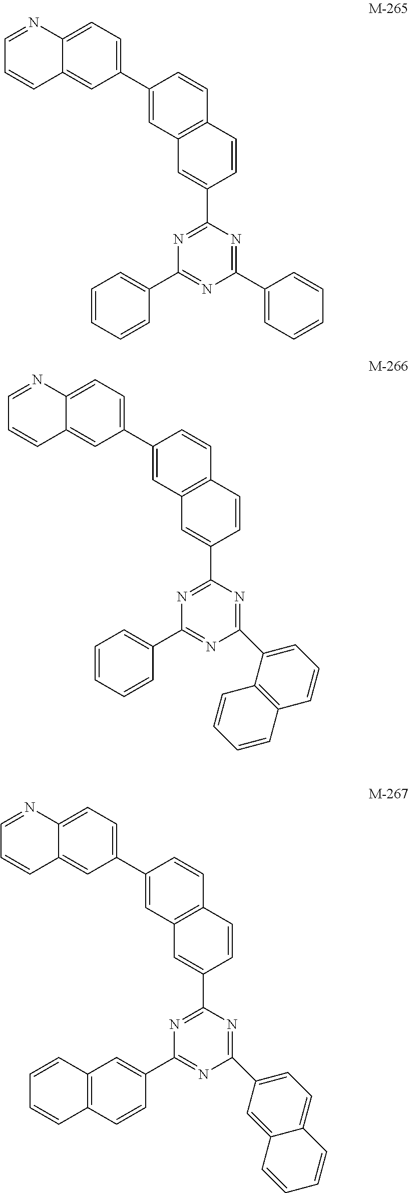 [SMILES: c1ccc(-c2nc(-c3ccccc3)nc(-c3ccc4ccc(-c5ccc6ncccc6c5)cc4c3)n2)cc1 ; c1ccc(-c2nc(-c3ccc4ccc(-c5ccc6ncccc6c5)cc4c3)nc(-c3cccc4ccccc34)n2)cc1 ; c1ccc2cc(-c3nc(-c4ccc5ccccc5c4)nc(-c4ccc5ccc(-c6ccc7ncccc7c6)cc5c4)n3)ccc2c1]

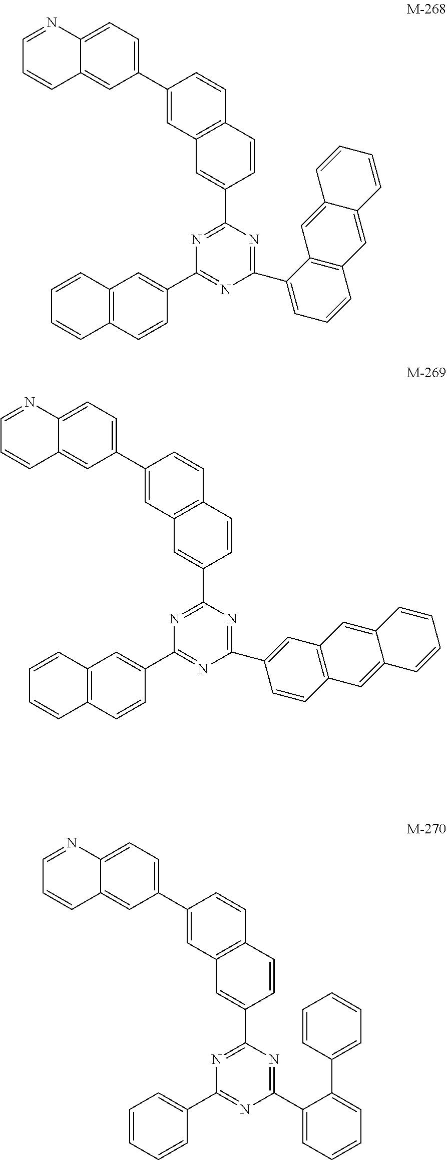 [SMILES: c1ccc2cc(-c3nc(-c4ccc5ccc(-c6ccc7ncccc7c6)cc5c4)nc(-c4cccc5cc6ccccc6cc45)n3)ccc2c1 ; c1ccc2cc(-c3nc(-c4ccc5ccc(-c6ccc7ncccc7c6)cc5c4)nc(-c4ccc5cc6ccccc6cc5c4)n3)ccc2c1 ; c1ccc(-c2nc(-c3ccc4ccc(-c5ccc6ncccc6c5)cc4c3)nc(-c3ccccc3-c3ccccc3)n2)cc1]

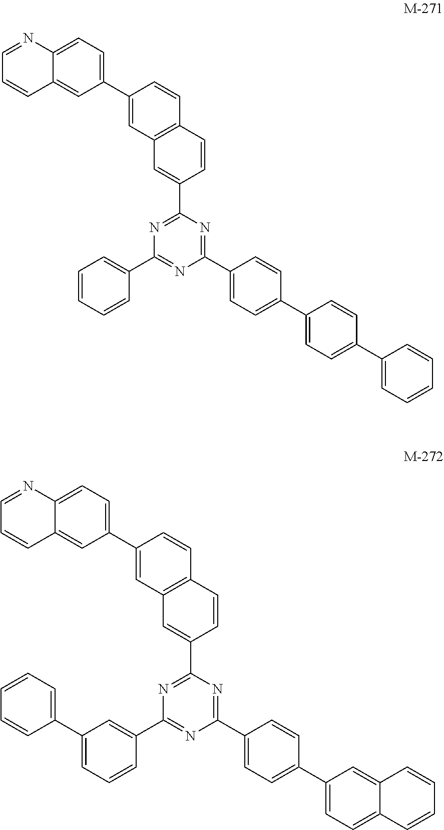 [SMILES: c1ccc(-c2ccc(-c3ccc(-c4nc(-c5ccccc5)nc(-c5ccc6ccc(-c7ccc8ncccc8c7)cc6c5)n4)cc3)cc2)cc1 ; c1ccc(-c2cccc(-c3nc(-c4ccc(-c5ccc6ccccc6c5)cc4)nc(-c4ccc5ccc(-c6ccc7ncccc7c6)cc5c4)n3)c2)cc1]

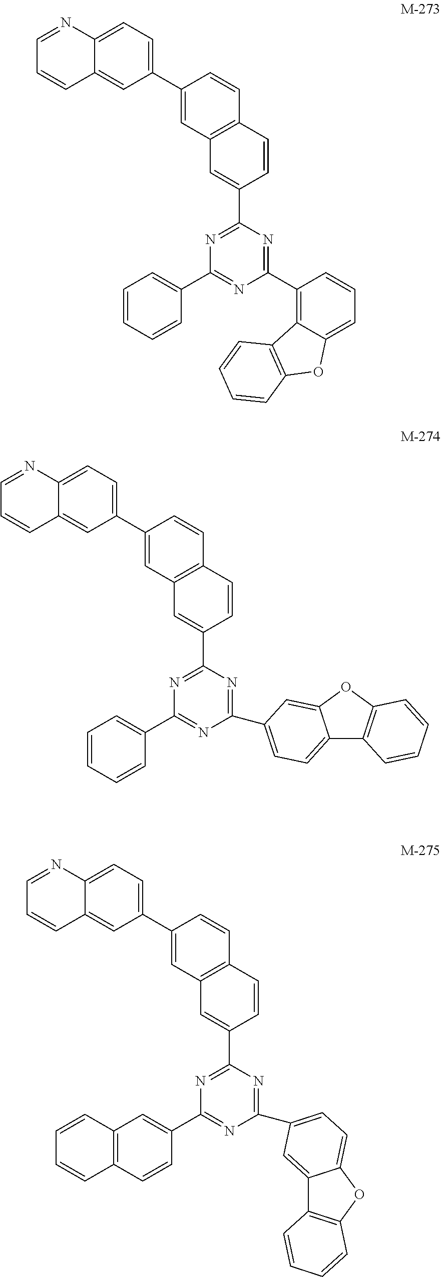 [SMILES: c1ccc(-c2nc(-c3ccc4ccc(-c5ccc6ncccc6c5)cc4c3)nc(-c3cccc4oc5ccccc5c34)n2)cc1 ; c1ccc(-c2nc(-c3ccc4ccc(-c5ccc6ncccc6c5)cc4c3)nc(-c3ccc4c(c3)oc3ccccc34)n2)cc1 ; c1ccc2cc(-c3nc(-c4ccc5ccc(-c6ccc7ncccc7c6)cc5c4)nc(-c4ccc5oc6ccccc6c5c4)n3)ccc2c1]

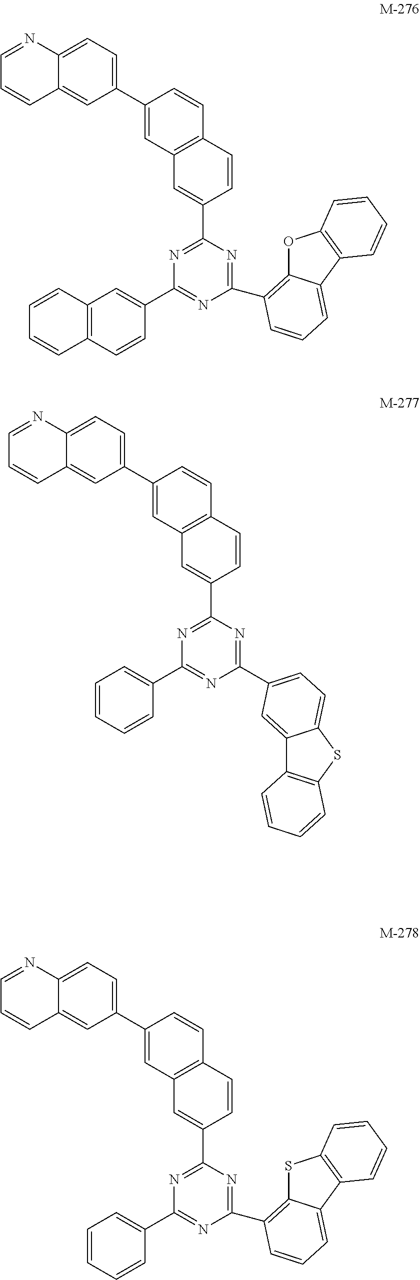 [SMILES: c1ccc2cc(-c3nc(-c4ccc5ccc(-c6ccc7ncccc7c6)cc5c4)nc(-c4cccc5c4oc4ccccc45)n3)ccc2c1 ; c1ccc(-c2nc(-c3ccc4ccc(-c5ccc6ncccc6c5)cc4c3)nc(-c3ccc4sc5ccccc5c4c3)n2)cc1 ; c1ccc(-c2nc(-c3ccc4ccc(-c5ccc6ncccc6c5)cc4c3)nc(-c3cccc4c3sc3ccccc34)n2)cc1]

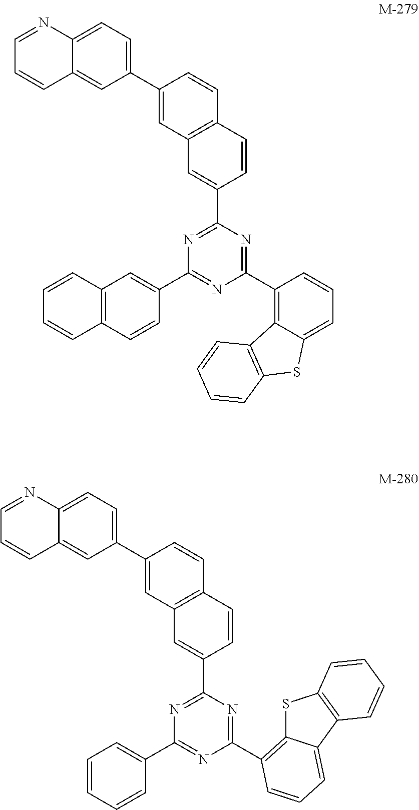 [SMILES: c1ccc2cc(-c3nc(-c4ccc5ccc(-c6ccc7ncccc7c6)cc5c4)nc(-c4cccc5sc6ccccc6c45)n3)ccc2c1 ; c1ccc(-c2nc(-c3ccc4ccc(-c5ccc6ncccc6c5)cc4c3)nc(-c3cccc4c3sc3ccccc34)n2)cc1]

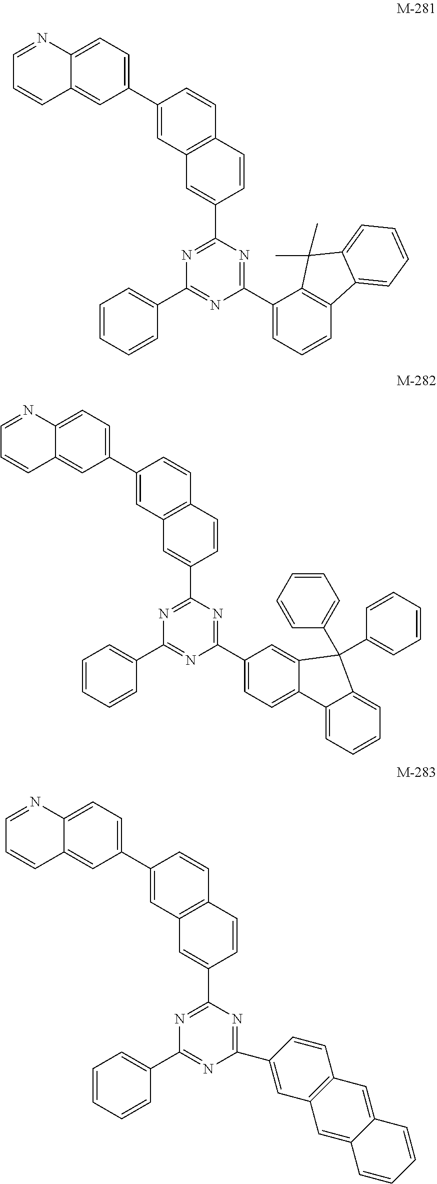 [SMILES: CC1(C)c2ccccc2-c2cccc(-c3nc(-c4ccccc4)nc(-c4ccc5ccc(-c6ccc7ncccc7c6)cc5c4)n3)c21 ; c1ccc(-c2nc(-c3ccc4c(c3)C(c3ccccc3)(c3ccccc3)c3ccccc3-4)nc(-c3ccc4ccc(-c5ccc6ncccc6c5)cc4c3)n2)cc1 ; c1ccc(-c2nc(-c3ccc4ccc(-c5ccc6ncccc6c5)cc4c3)nc(-c3ccc4cc5ccccc5cc4c3)n2)cc1]

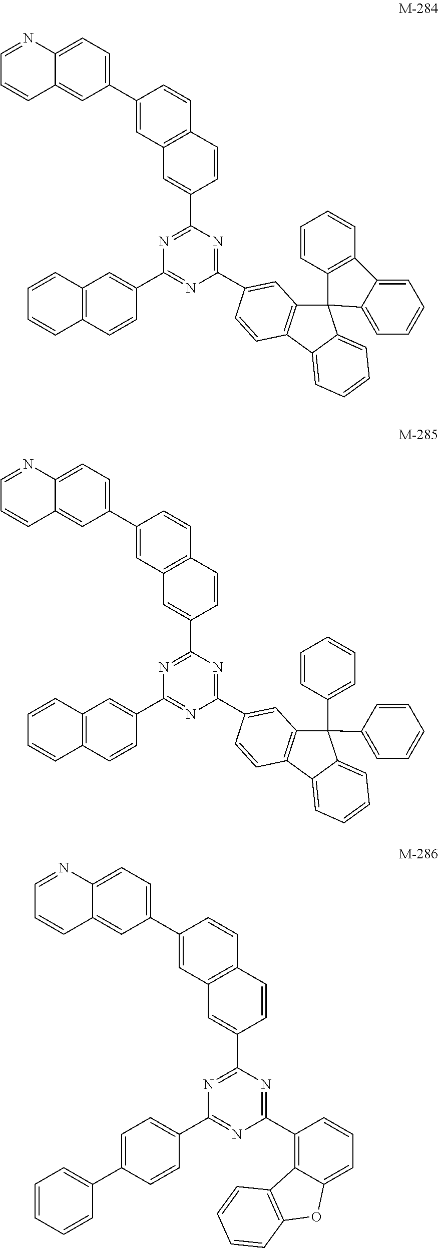 [SMILES: c1ccc2c(c1)-c1ccccc1C21c2ccccc2-c2ccc(-c3nc(-c4ccc5ccccc5c4)nc(-c4ccc5ccc(-c6ccc7ncccc7c6)cc5c4)n3)cc21 ; c1ccc(C2(c3ccccc3)c3ccccc3-c3ccc(-c4nc(-c5ccc6ccccc6c5)nc(-c5ccc6ccc(-c7ccc8ncccc8c7)cc6c5)n4)cc32)cc1 ; c1ccc(-c2ccc(-c3nc(-c4ccc5ccc(-c6ccc7ncccc7c6)cc5c4)nc(-c4cccc5oc6ccccc6c45)n3)cc2)cc1]

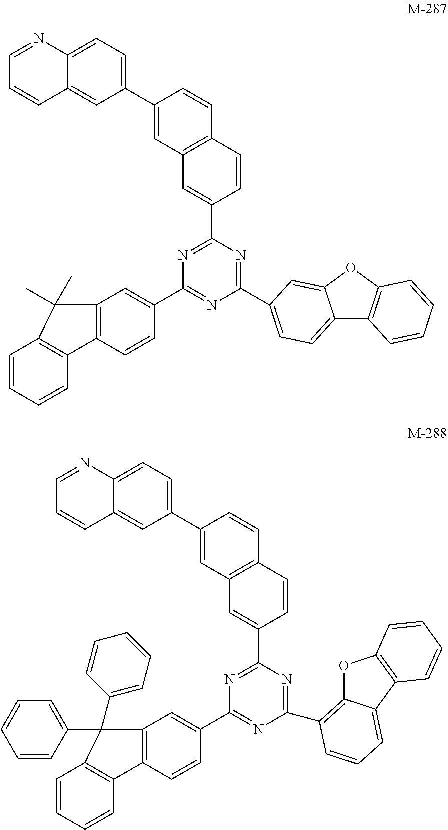 [SMILES: CC1(C)c2ccccc2-c2ccc(-c3nc(-c4ccc5ccc(-c6ccc7ncccc7c6)cc5c4)nc(-c4ccc5c(c4)oc4ccccc45)n3)cc21 ; c1ccc(C2(c3ccccc3)c3ccccc3-c3ccc(-c4nc(-c5ccc6ccc(-c7ccc8ncccc8c7)cc6c5)nc(-c5cccc6c5oc5ccccc56)n4)cc32)cc1]

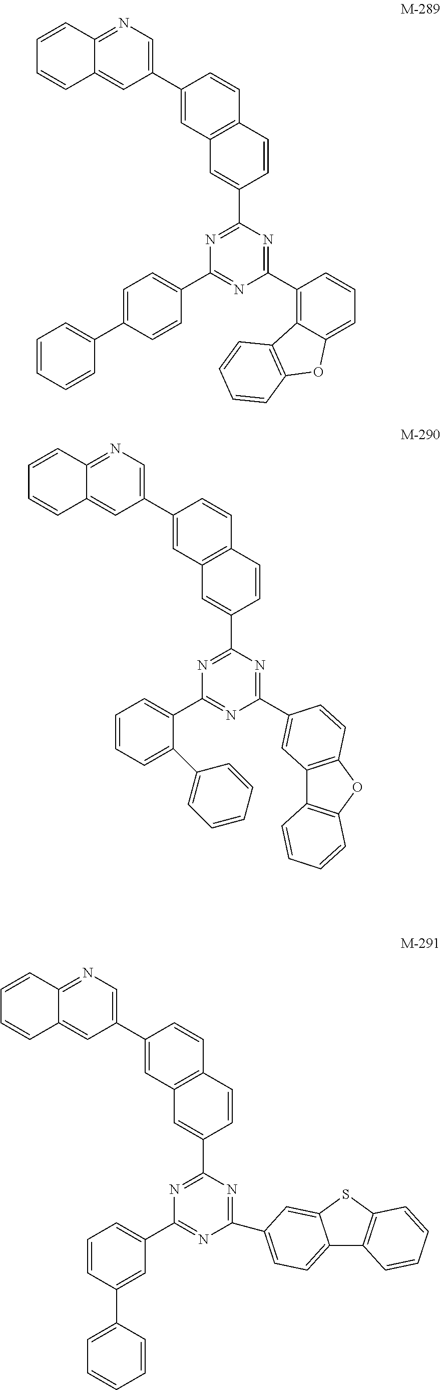 [SMILES: c1ccc(-c2ccc(-c3nc(-c4ccc5ccc(-c6cnc7ccccc7c6)cc5c4)nc(-c4cccc5oc6ccccc6c45)n3)cc2)cc1 ; c1ccc(-c2ccccc2-c2nc(-c3ccc4ccc(-c5cnc6ccccc6c5)cc4c3)nc(-c3ccc4oc5ccccc5c4c3)n2)cc1 ; c1ccc(-c2cccc(-c3nc(-c4ccc5ccc(-c6cnc7ccccc7c6)cc5c4)nc(-c4ccc5c(c4)sc4ccccc45)n3)c2)cc1]

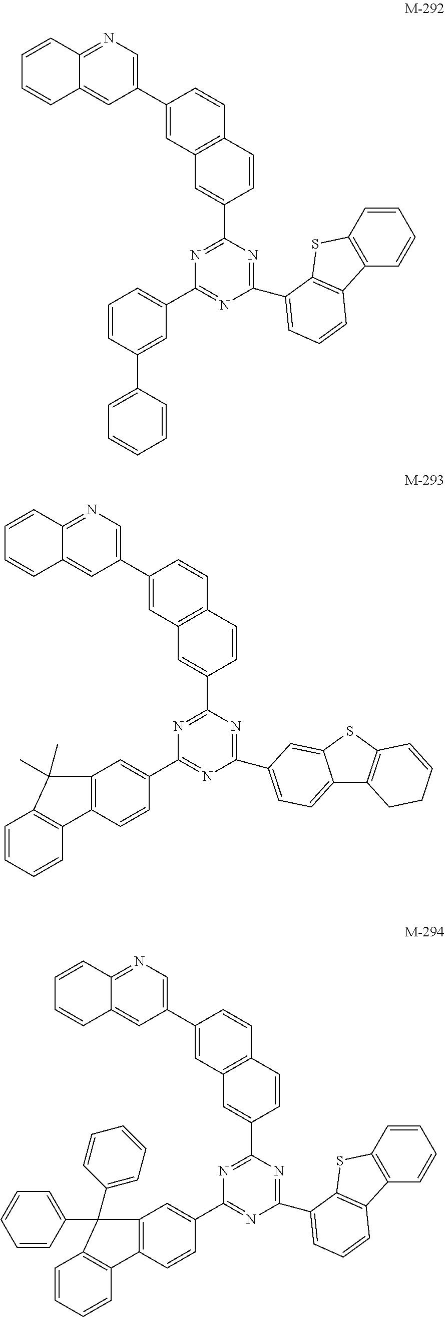 [SMILES: c1ccc(-c2cccc(-c3nc(-c4ccc5ccc(-c6cnc7ccccc7c6)cc5c4)nc(-c4cccc5c4sc4ccccc45)n3)c2)cc1 ; CC1(C)c2ccccc2-c2ccc(-c3nc(-c4ccc5ccc(-c6cnc7ccccc7c6)cc5c4)nc(-c4ccc5c6c(sc5c4)C=CCC6)n3)cc21 ; c1ccc(C2(c3ccccc3)c3ccccc3-c3ccc(-c4nc(-c5ccc6ccc(-c7cnc8ccccc8c7)cc6c5)nc(-c5cccc6c5sc5ccccc56)n4)cc32)cc1]

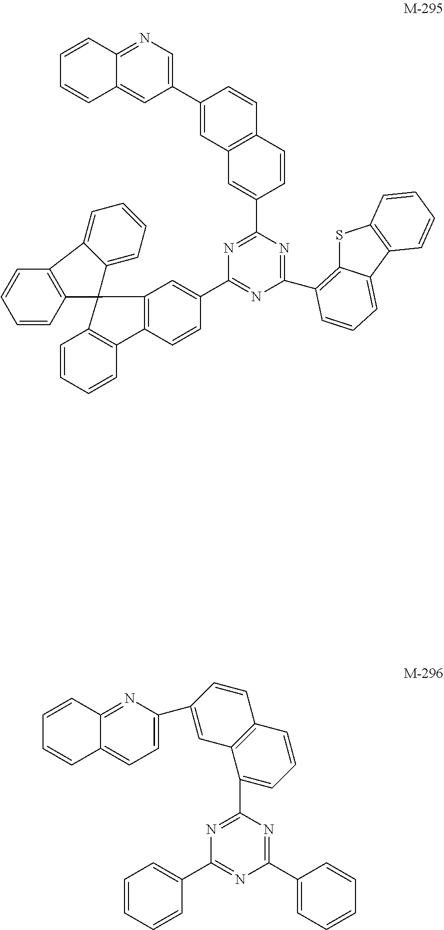 [SMILES: c1ccc2c(c1)-c1ccccc1C21c2ccccc2-c2ccc(-c3nc(-c4ccc5ccc(-c6cnc7ccccc7c6)cc5c4)nc(-c4cccc5c4sc4ccccc45)n3)cc21 ; c1ccc(-c2nc(-c3ccccc3)nc(-c3cccc4ccc(-c5ccc6ccccc6n5)cc34)n2)cc1]

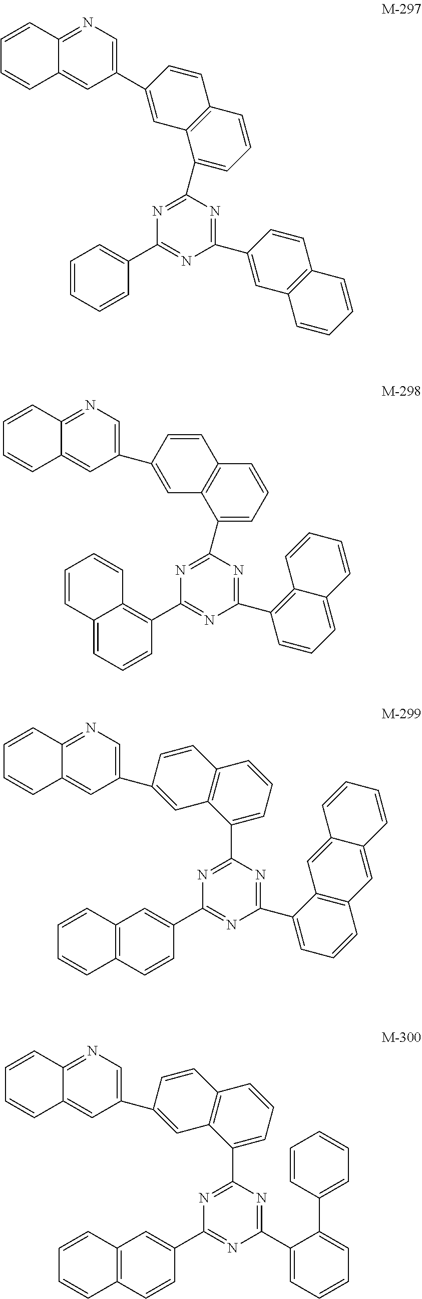 [SMILES: c1ccc(-c2nc(-c3ccc4ccccc4c3)nc(-c3cccc4ccc(-c5cnc6ccccc6c5)cc34)n2)cc1 ; c1ccc2ncc(-c3ccc4cccc(-c5nc(-c6cccc7ccccc67)nc(-c6cccc7ccccc67)n5)c4c3)cc2c1 ; c1ccc2cc(-c3nc(-c4cccc5ccc(-c6cnc7ccccc7c6)cc45)nc(-c4cccc5cc6ccccc6cc45)n3)ccc2c1 ; c1ccc(-c2ccccc2-c2nc(-c3ccc4ccccc4c3)nc(-c3cccc4ccc(-c5cnc6ccccc6c5)cc34)n2)cc1]

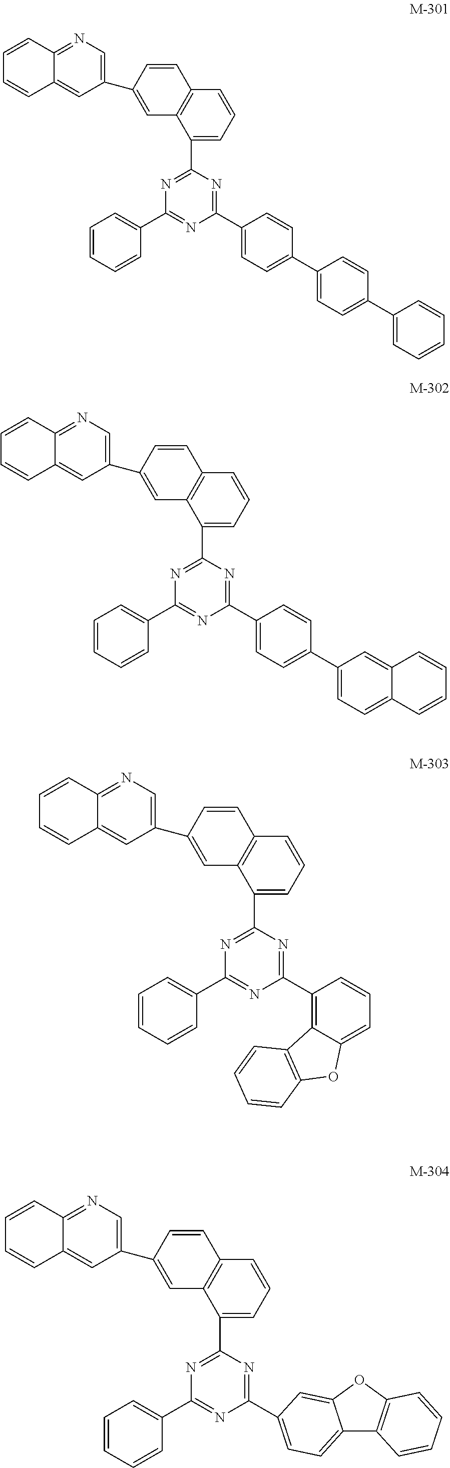 [SMILES: c1ccc(-c2ccc(-c3ccc(-c4nc(-c5ccccc5)nc(-c5cccc6ccc(-c7cnc8ccccc8c7)cc56)n4)cc3)cc2)cc1 ; c1ccc(-c2nc(-c3ccc(-c4ccc5ccccc5c4)cc3)nc(-c3cccc4ccc(-c5cnc6ccccc6c5)cc34)n2)cc1 ; c1ccc(-c2nc(-c3cccc4ccc(-c5cnc6ccccc6c5)cc34)nc(-c3cccc4oc5ccccc5c34)n2)cc1 ; c1ccc(-c2nc(-c3ccc4c(c3)oc3ccccc34)nc(-c3cccc4ccc(-c5cnc6ccccc6c5)cc34)n2)cc1]

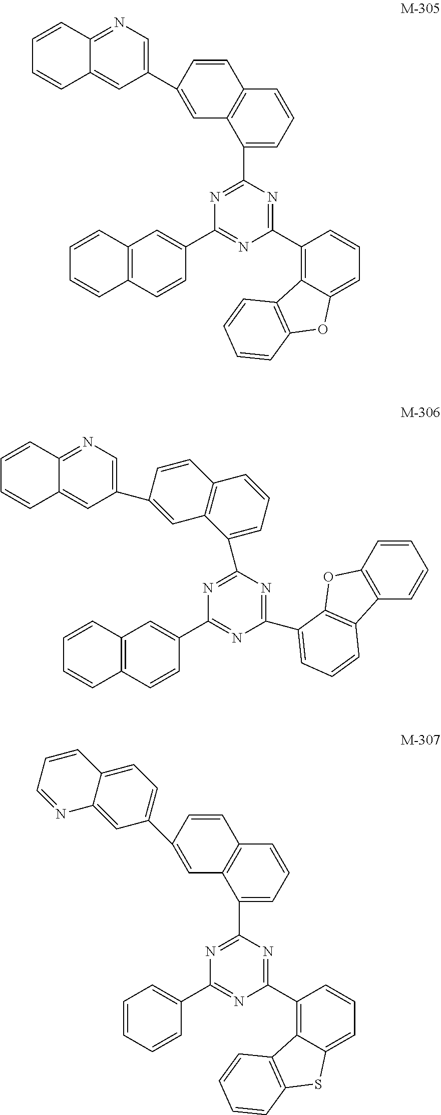 [SMILES: c1ccc2cc(-c3nc(-c4cccc5ccc(-c6cnc7ccccc7c6)cc45)nc(-c4cccc5oc6ccccc6c45)n3)ccc2c1 ; c1ccc2cc(-c3nc(-c4cccc5ccc(-c6cnc7ccccc7c6)cc45)nc(-c4cccc5c4oc4ccccc45)n3)ccc2c1 ; c1ccc(-c2nc(-c3cccc4ccc(-c5ccc6cccnc6c5)cc34)nc(-c3cccc4sc5ccccc5c34)n2)cc1]

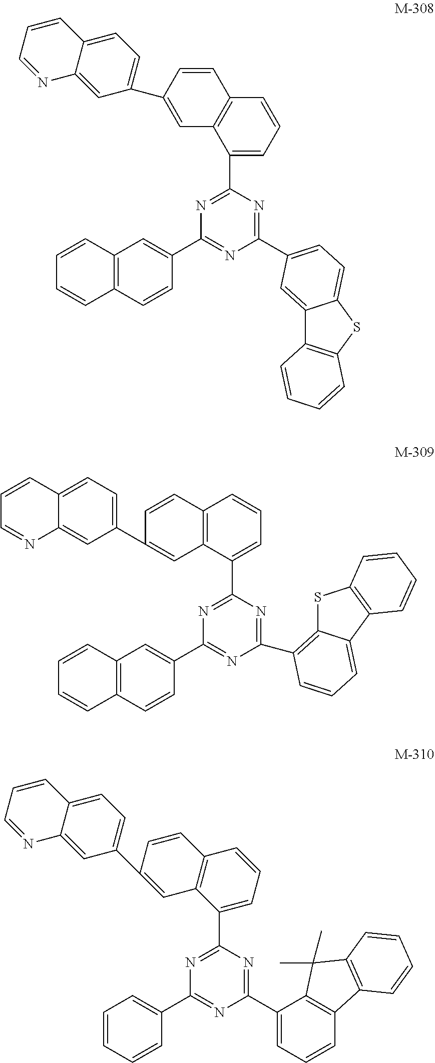 [SMILES: c1ccc2cc(-c3nc(-c4ccc5sc6ccccc6c5c4)nc(-c4cccc5ccc(-c6ccc7cccnc7c6)cc45)n3)ccc2c1 ; c1ccc2cc(-c3nc(-c4cccc5ccc(-c6ccc7cccnc7c6)cc45)nc(-c4cccc5c4sc4ccccc45)n3)ccc2c1 ; CC1(C)c2ccccc2-c2cccc(-c3nc(-c4ccccc4)nc(-c4cccc5ccc(-c6ccc7cccnc7c6)cc45)n3)c21]

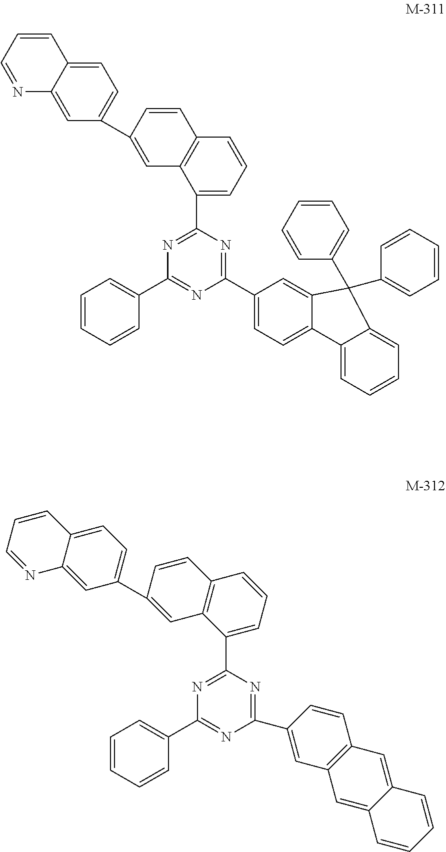 [SMILES: c1ccc(-c2nc(-c3ccc4c(c3)C(c3ccccc3)(c3ccccc3)c3ccccc3-4)nc(-c3cccc4ccc(-c5ccc6cccnc6c5)cc34)n2)cc1 ; c1ccc(-c2nc(-c3ccc4cc5ccccc5cc4c3)nc(-c3cccc4ccc(-c5ccc6cccnc6c5)cc34)n2)cc1]

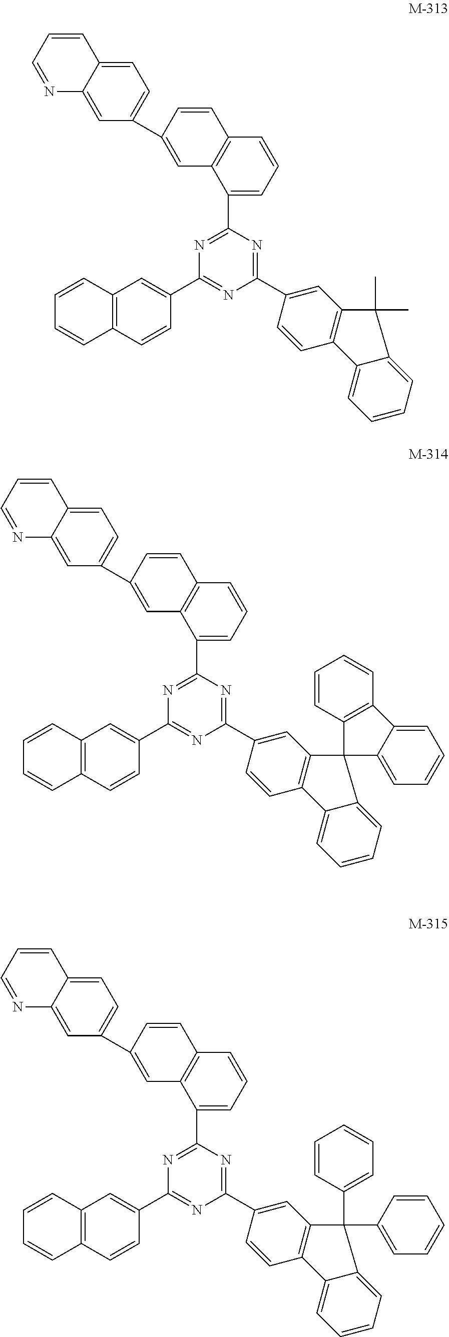 [SMILES: CC1(C)c2ccccc2-c2ccc(-c3nc(-c4ccc5ccccc5c4)nc(-c4cccc5ccc(-c6ccc7cccnc7c6)cc45)n3)cc21 ; c1ccc2c(c1)-c1ccccc1C21c2ccccc2-c2ccc(-c3nc(-c4ccc5ccccc5c4)nc(-c4cccc5ccc(-c6ccc7cccnc7c6)cc45)n3)cc21 ; c1ccc(C2(c3ccccc3)c3ccccc3-c3ccc(-c4nc(-c5ccc6ccccc6c5)nc(-c5cccc6ccc(-c7ccc8cccnc8c7)cc56)n4)cc32)cc1]

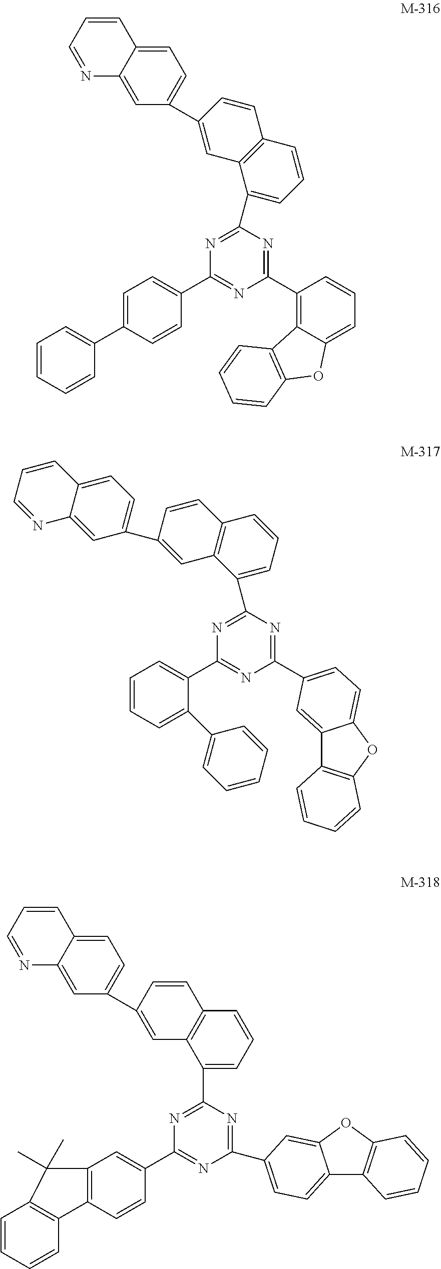 [SMILES: c1ccc(-c2ccc(-c3nc(-c4cccc5ccc(-c6ccc7cccnc7c6)cc45)nc(-c4cccc5oc6ccccc6c45)n3)cc2)cc1 ; c1ccc(-c2ccccc2-c2nc(-c3ccc4oc5ccccc5c4c3)nc(-c3cccc4ccc(-c5ccc6cccnc6c5)cc34)n2)cc1 ; CC1(C)c2ccccc2-c2ccc(-c3nc(-c4ccc5c(c4)oc4ccccc45)nc(-c4cccc5ccc(-c6ccc7cccnc7c6)cc45)n3)cc21]

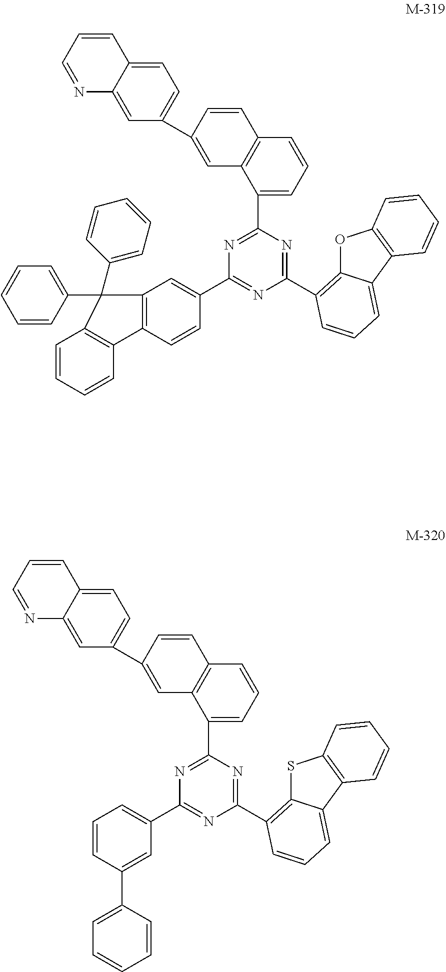 [SMILES: c1ccc(C2(c3ccccc3)c3ccccc3-c3ccc(-c4nc(-c5cccc6ccc(-c7ccc8cccnc8c7)cc56)nc(-c5cccc6c5oc5ccccc56)n4)cc32)cc1 ; c1ccc(-c2cccc(-c3nc(-c4cccc5ccc(-c6ccc7cccnc7c6)cc45)nc(-c4cccc5c4sc4ccccc45)n3)c2)cc1]

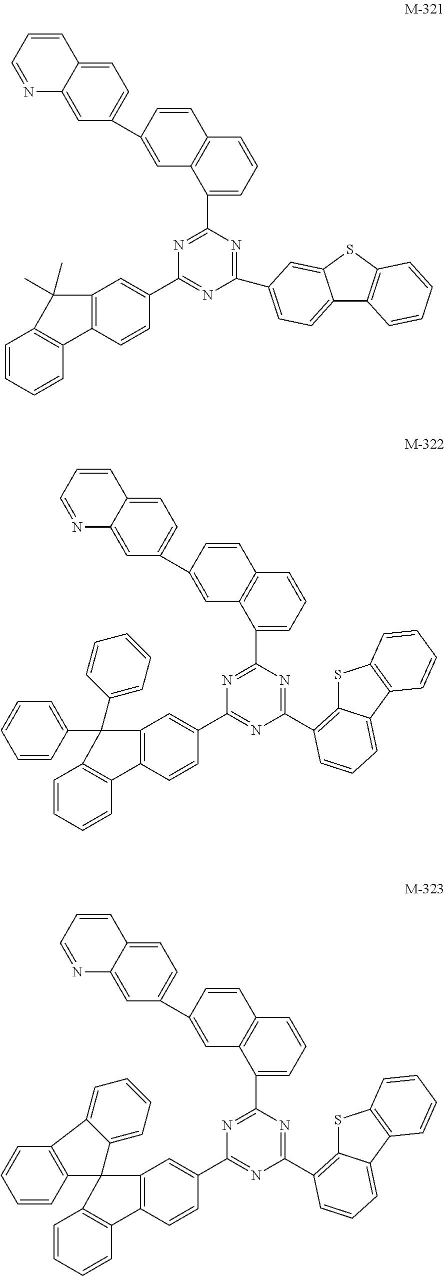 [SMILES: CC1(C)c2ccccc2-c2ccc(-c3nc(-c4ccc5c(c4)sc4ccccc45)nc(-c4cccc5ccc(-c6ccc7cccnc7c6)cc45)n3)cc21 ; c1ccc(C2(c3ccccc3)c3ccccc3-c3ccc(-c4nc(-c5cccc6ccc(-c7ccc8cccnc8c7)cc56)nc(-c5cccc6c5sc5ccccc56)n4)cc32)cc1 ; c1ccc2c(c1)-c1ccccc1C21c2ccccc2-c2ccc(-c3nc(-c4cccc5ccc(-c6ccc7cccnc7c6)cc45)nc(-c4cccc5c4sc4ccccc45)n3)cc21]

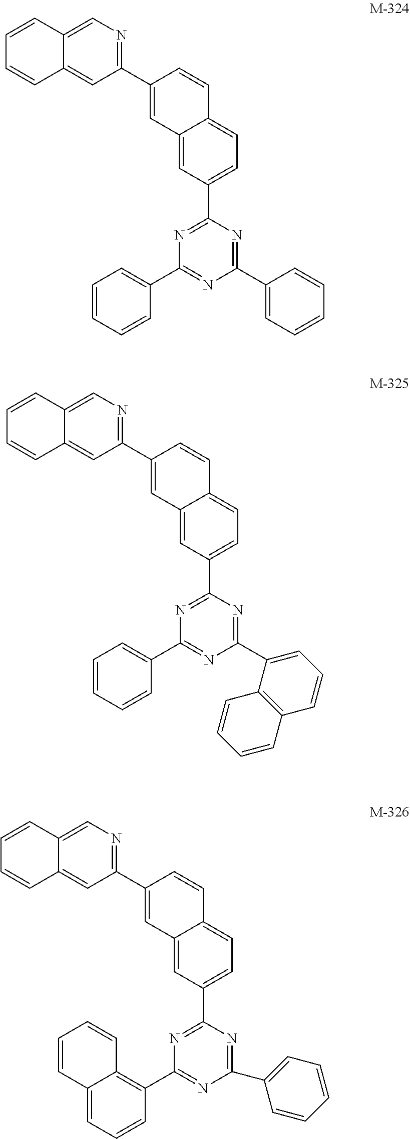 [SMILES: c1ccc(-c2nc(-c3ccccc3)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3cccc4ccccc34)n2)cc1 ; c1ccc(-c2nc(-c3ccc4ccc(-c5cc6ccccc6cn5)cc4c3)nc(-c3cccc4ccccc34)n2)cc1]

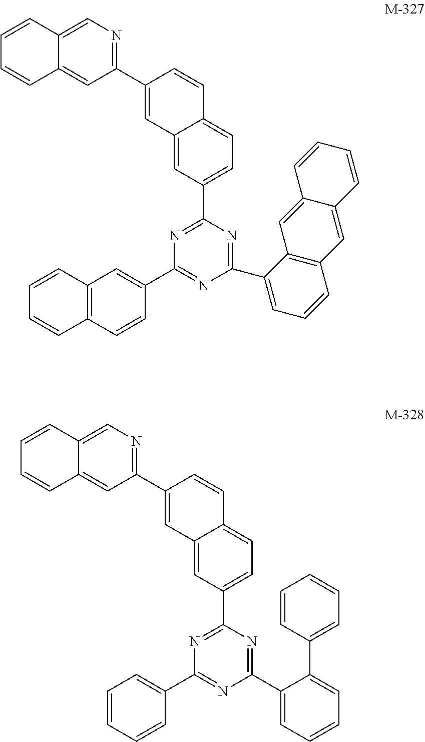 [SMILES: c1ccc2cc(-c3nc(-c4ccc5ccc(-c6cc7ccccc7cn6)cc5c4)nc(-c4cccc5cc6ccccc6cc45)n3)ccc2c1 ; c1ccc(-c2nc(-c3ccc4ccc(-c5cc6ccccc6cn5)cc4c3)nc(-c3ccccc3-c3ccccc3)n2)cc1]

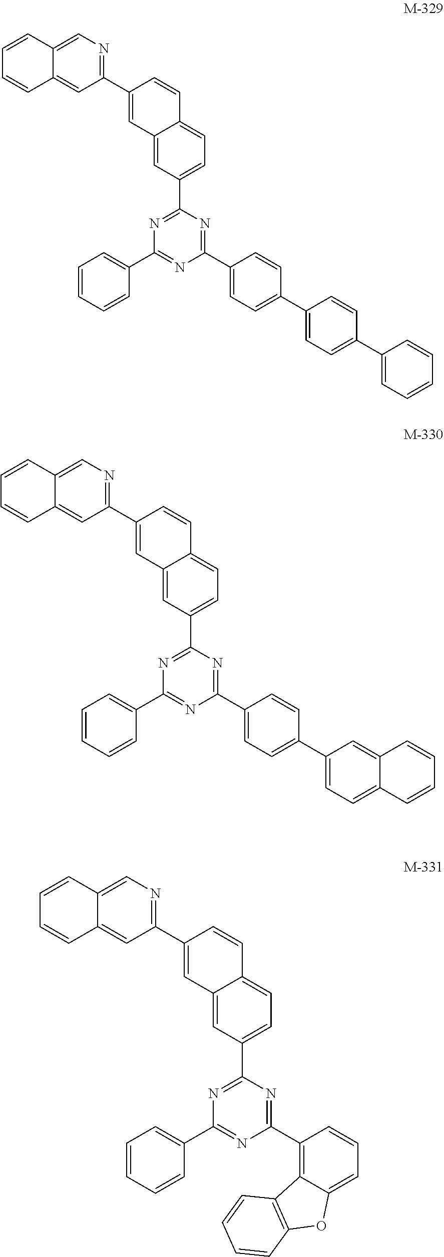 [SMILES: c1ccc(-c2ccc(-c3ccc(-c4nc(-c5ccccc5)nc(-c5ccc6ccc(-c7cc8ccccc8cn7)cc6c5)n4)cc3)cc2)cc1 ; c1ccc(-c2nc(-c3ccc(-c4ccc5ccccc5c4)cc3)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3cccc4oc5ccccc5c34)n2)cc1]

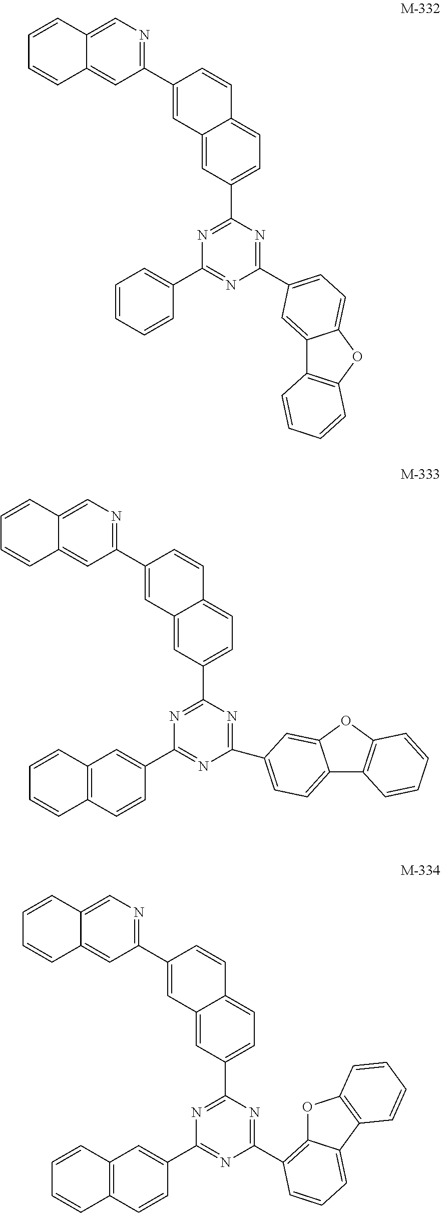 [SMILES: c1ccc(-c2nc(-c3ccc4ccc(-c5cc6ccccc6cn5)cc4c3)nc(-c3ccc4oc5ccccc5c4c3)n2)cc1 ; c1ccc2cc(-c3nc(-c4ccc5ccc(-c6cc7ccccc7cn6)cc5c4)nc(-c4ccc5c(c4)oc4ccccc45)n3)ccc2c1 ; c1ccc2cc(-c3nc(-c4ccc5ccc(-c6cc7ccccc7cn6)cc5c4)nc(-c4cccc5c4oc4ccccc45)n3)ccc2c1]

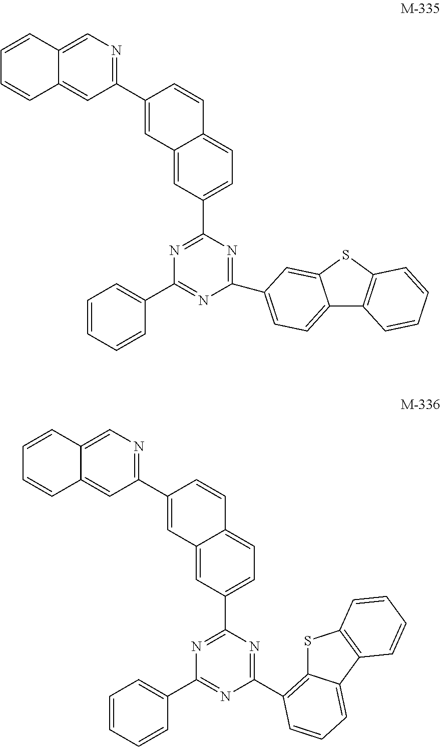 [SMILES: c1ccc(-c2nc(-c3ccc4ccc(-c5cc6ccccc6cn5)cc4c3)nc(-c3ccc4c(c3)sc3ccccc34)n2)cc1 ; c1ccc(-c2nc(-c3ccc4ccc(-c5cc6ccccc6cn5)cc4c3)nc(-c3cccc4c3sc3ccccc34)n2)cc1]

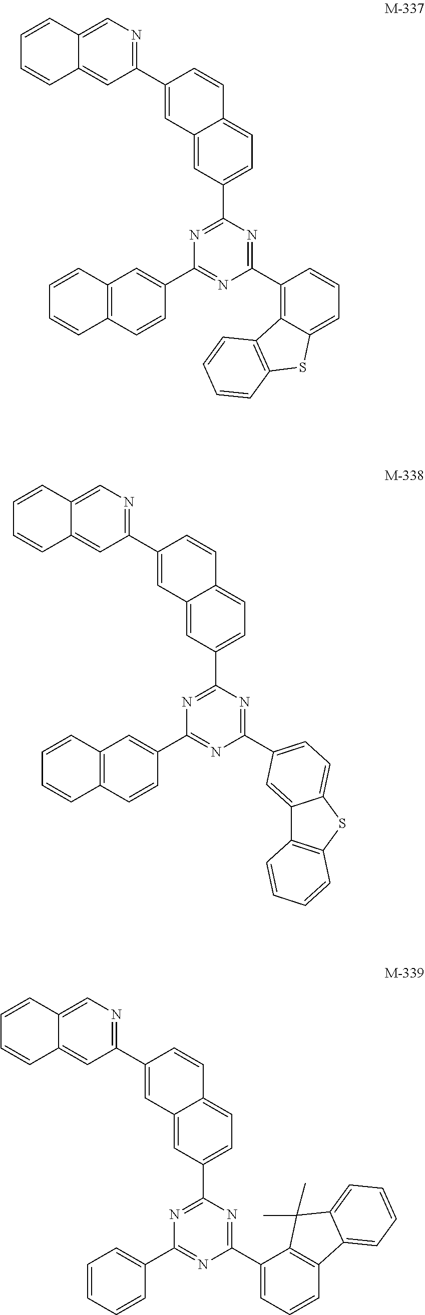 [SMILES: c1ccc2cc(-c3nc(-c4ccc5ccc(-c6cc7ccccc7cn6)cc5c4)nc(-c4cccc5sc6ccccc6c45)n3)ccc2c1 ; c1ccc2cc(-c3nc(-c4ccc5ccc(-c6cc7ccccc7cn6)cc5c4)nc(-c4ccc5sc6ccccc6c5c4)n3)ccc2c1 ; CC1(C)c2ccccc2-c2cccc(-c3nc(-c4ccccc4)nc(-c4ccc5ccc(-c6cc7ccccc7cn6)cc5c4)n3)c21]

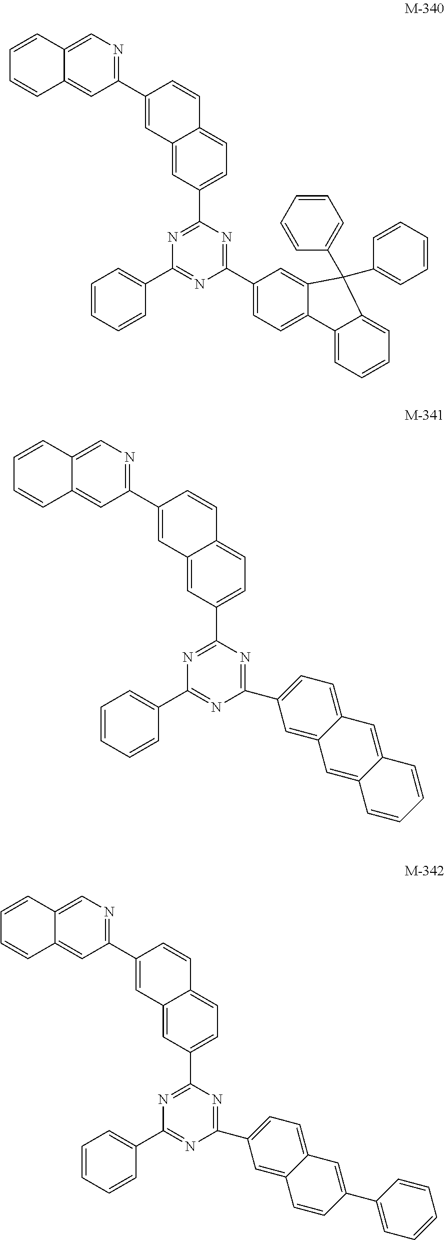 [SMILES: c1ccc(-c2nc(-c3ccc4c(c3)C(c3ccccc3)(c3ccccc3)c3ccccc3-4)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3ccc4cc5ccccc5cc4c3)n2)cc1 ; c1ccc(-c2ccc3cc(-c4nc(-c5ccccc5)nc(-c5ccc6ccc(-c7cc8ccccc8cn7)cc6c5)n4)ccc3c2)cc1]

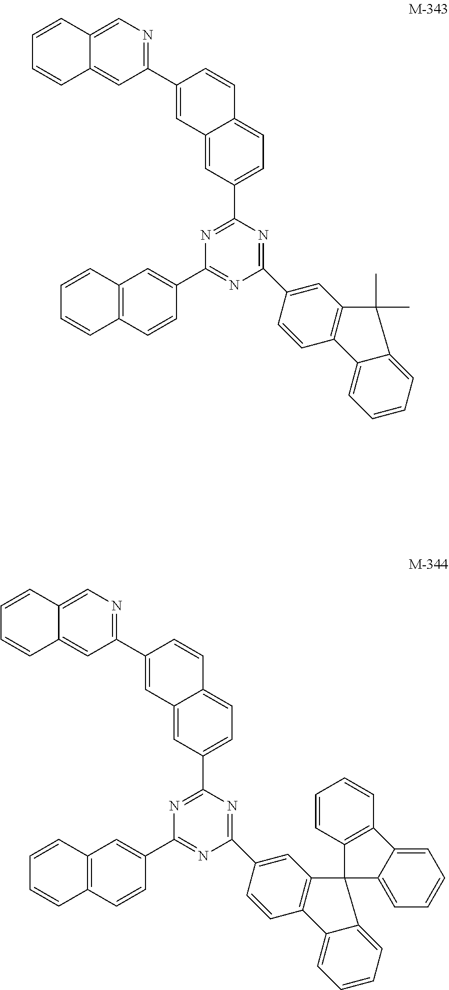 [SMILES: CC1(C)c2ccccc2-c2ccc(-c3nc(-c4ccc5ccccc5c4)nc(-c4ccc5ccc(-c6cc7ccccc7cn6)cc5c4)n3)cc21 ; c1ccc2c(c1)-c1ccccc1C21c2ccccc2-c2ccc(-c3nc(-c4ccc5ccccc5c4)nc(-c4ccc5ccc(-c6cc7ccccc7cn6)cc5c4)n3)cc21]

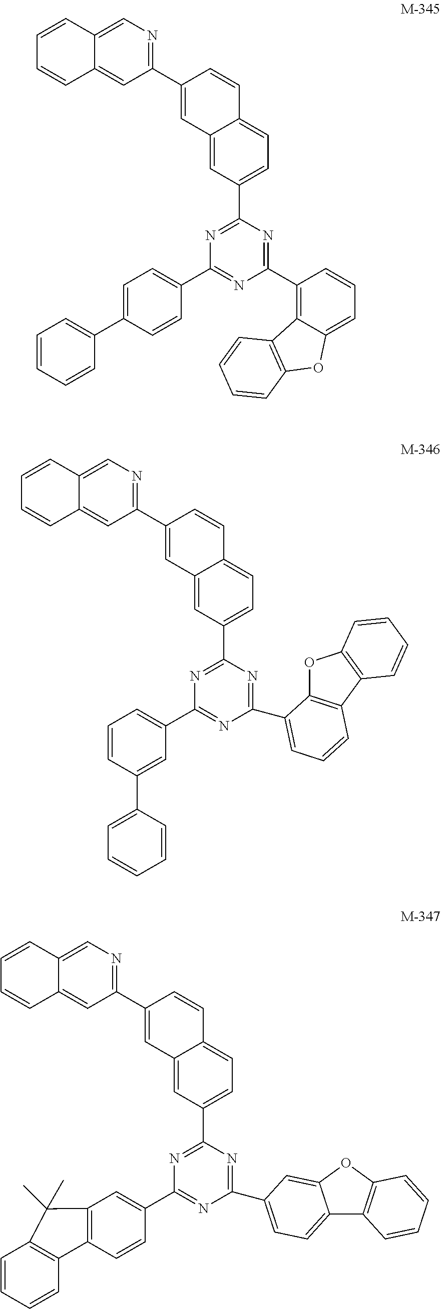 [SMILES: c1ccc(-c2ccc(-c3nc(-c4ccc5ccc(-c6cc7ccccc7cn6)cc5c4)nc(-c4cccc5oc6ccccc6c45)n3)cc2)cc1 ; c1ccc(-c2cccc(-c3nc(-c4ccc5ccc(-c6cc7ccccc7cn6)cc5c4)nc(-c4cccc5c4oc4ccccc45)n3)c2)cc1 ; CC1(C)c2ccccc2-c2ccc(-c3nc(-c4ccc5ccc(-c6cc7ccccc7cn6)cc5c4)nc(-c4ccc5c(c4)oc4ccccc45)n3)cc21]

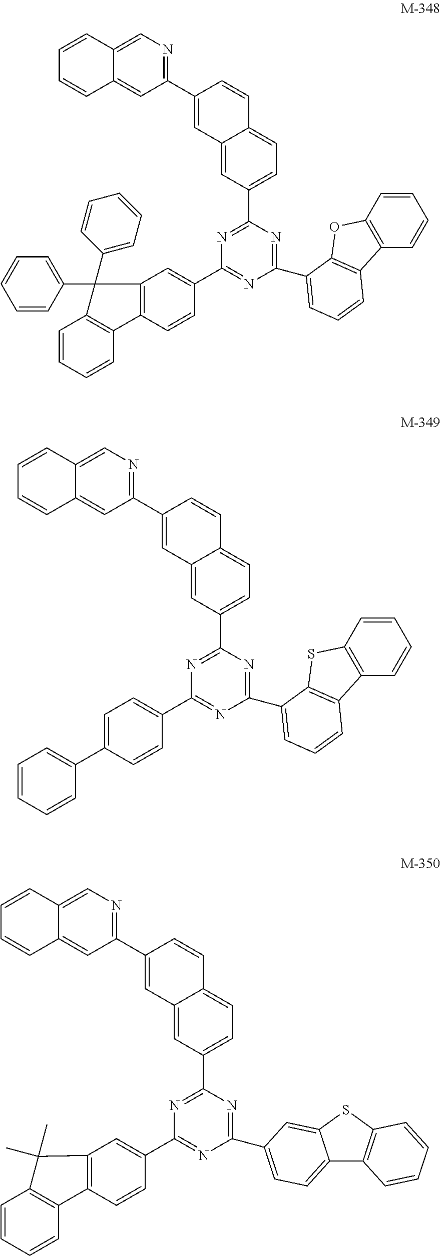 [SMILES: c1ccc(C2(c3ccccc3)c3ccccc3-c3ccc(-c4nc(-c5ccc6ccc(-c7cc8ccccc8cn7)cc6c5)nc(-c5cccc6c5oc5ccccc56)n4)cc32)cc1 ; c1ccc(-c2ccc(-c3nc(-c4ccc5ccc(-c6cc7ccccc7cn6)cc5c4)nc(-c4cccc5c4sc4ccccc45)n3)cc2)cc1 ; CC1(C)c2ccccc2-c2ccc(-c3nc(-c4ccc5ccc(-c6cc7ccccc7cn6)cc5c4)nc(-c4ccc5c(c4)sc4ccccc45)n3)cc21]

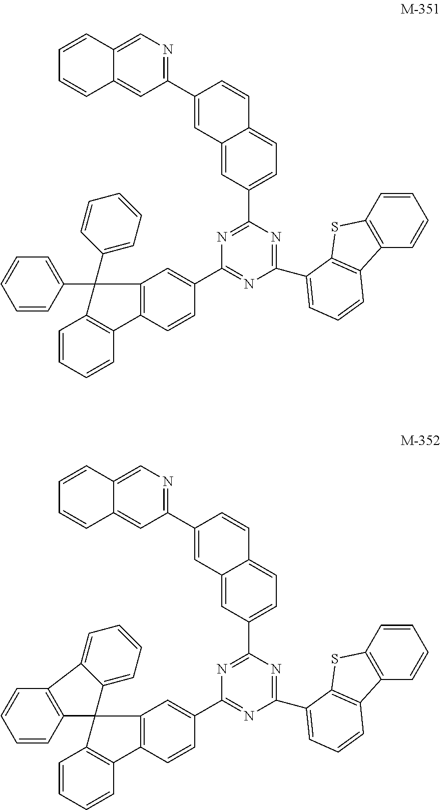 [SMILES: c1ccc(C2(c3ccccc3)c3ccccc3-c3ccc(-c4nc(-c5ccc6ccc(-c7cc8ccccc8cn7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6cc7ccccc7cn6)cc5c4)nc(-c4cccc5c4sc4ccccc45)n3)cc21]

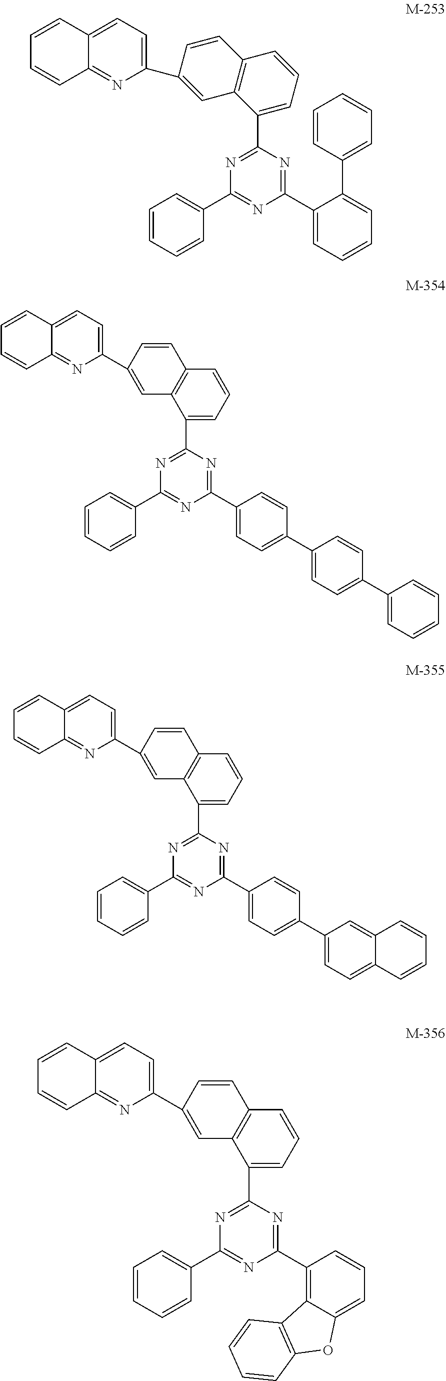 [SMILES: c1ccc(-c2nc(-c3ccccc3-c3ccccc3)nc(-c3cccc4ccc(-c5ccc6ccccc6n5)cc34)n2)cc1 ; c1ccc(-c2ccc(-c3ccc(-c4nc(-c5ccccc5)nc(-c5cccc6ccc(-c7ccc8ccccc8n7)cc56)n4)cc3)cc2)cc1 ; c1ccc(-c2nc(-c3ccc(-c4ccc5ccccc5c4)cc3)nc(-c3cccc4ccc(-c5ccc6ccccc6n5)cc34)n2)cc1 ; c1ccc(-c2nc(-c3cccc4ccc(-c5ccc6ccccc6n5)cc34)nc(-c3cccc4oc5ccccc5c34)n2)cc1]

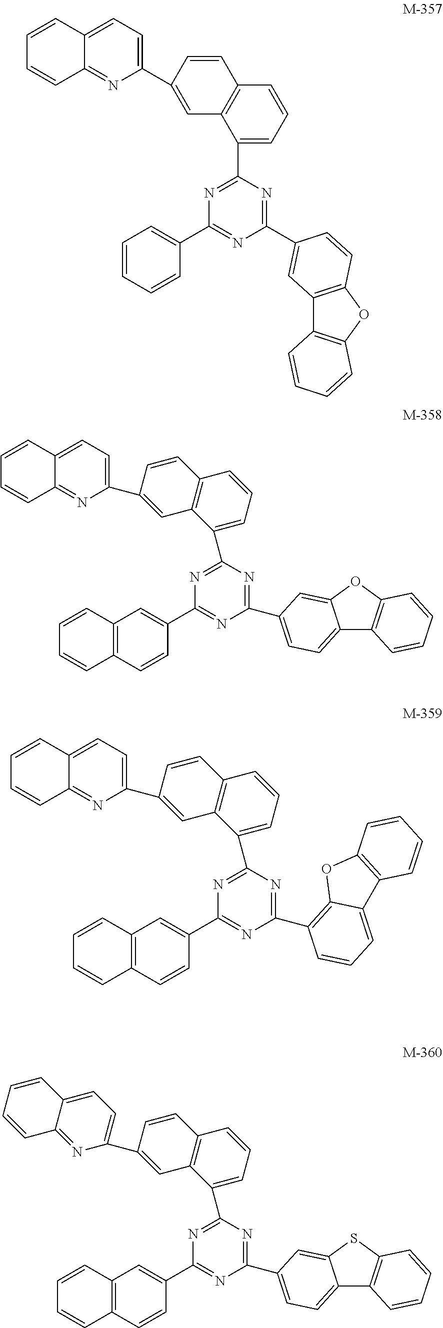 [SMILES: c1ccc(-c2nc(-c3ccc4oc5ccccc5c4c3)nc(-c3cccc4ccc(-c5ccc6ccccc6n5)cc34)n2)cc1 ; c1ccc2cc(-c3nc(-c4ccc5c(c4)oc4ccccc45)nc(-c4cccc5ccc(-c6ccc7ccccc7n6)cc45)n3)ccc2c1 ; c1ccc2cc(-c3nc(-c4cccc5ccc(-c6ccc7ccccc7n6)cc45)nc(-c4cccc5c4oc4ccccc45)n3)ccc2c1 ; c1ccc2cc(-c3nc(-c4ccc5c(c4)sc4ccccc45)nc(-c4cccc5ccc(-c6ccc7ccccc7n6)cc45)n3)ccc2c1]

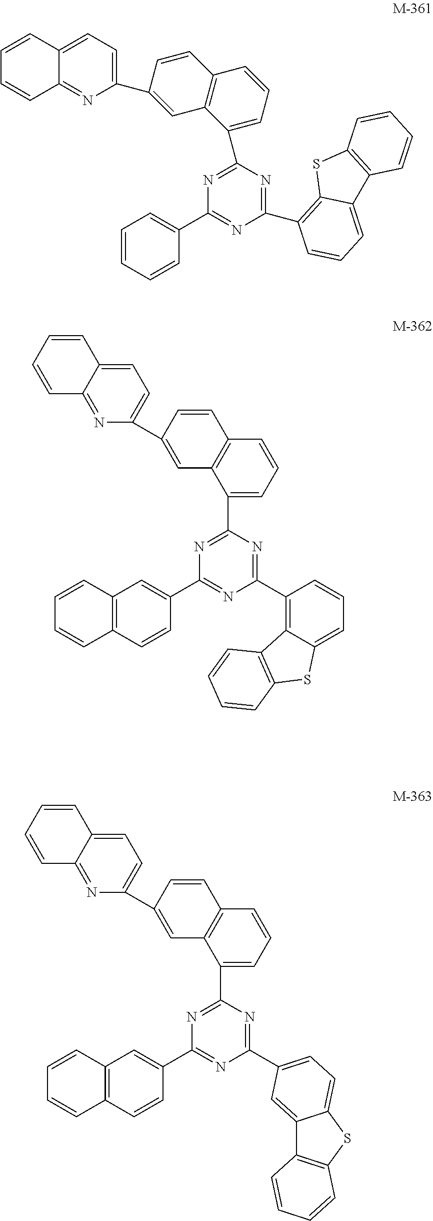 [SMILES: c1ccc(-c2nc(-c3cccc4ccc(-c5ccc6ccccc6n5)cc34)nc(-c3cccc4c3sc3ccccc34)n2)cc1 ; c1ccc2cc(-c3nc(-c4cccc5ccc(-c6ccc7ccccc7n6)cc45)nc(-c4cccc5sc6ccccc6c45)n3)ccc2c1 ; c1ccc2cc(-c3nc(-c4ccc5sc6ccccc6c5c4)nc(-c4cccc5ccc(-c6ccc7ccccc7n6)cc45)n3)ccc2c1]

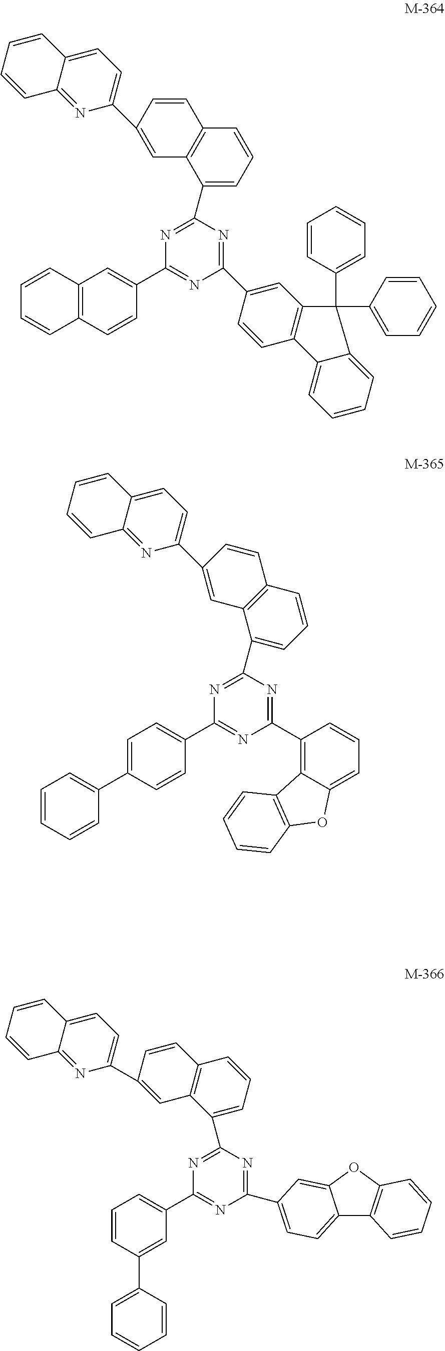 [SMILES: c1ccc(C2(c3ccccc3)c3ccccc3-c3ccc(-c4nc(-c5ccc6ccccc6c5)nc(-c5cccc6ccc(-c7ccc8ccccc8n7)cc56)n4)cc32)cc1 ; c1ccc(-c2ccc(-c3nc(-c4cccc5ccc(-c6ccc7ccccc7n6)cc45)nc(-c4cccc5oc6ccccc6c45)n3)cc2)cc1 ; c1ccc(-c2cccc(-c3nc(-c4ccc5c(c4)oc4ccccc45)nc(-c4cccc5ccc(-c6ccc7ccccc7n6)cc45)n3)c2)cc1]

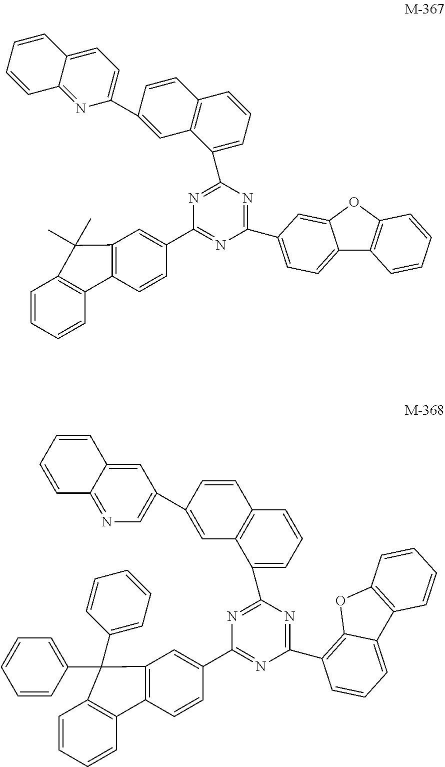 [SMILES: CC1(C)c2ccccc2-c2ccc(-c3nc(-c4ccc5c(c4)oc4ccccc45)nc(-c4cccc5ccc(-c6ccc7ccccc7n6)cc45)n3)cc21 ; c1ccc(C2(c3ccccc3)c3ccccc3-c3ccc(-c4nc(-c5cccc6ccc(-c7cnc8ccccc8c7)cc56)nc(-c5cccc6c5oc5ccccc56)n4)cc32)cc1]

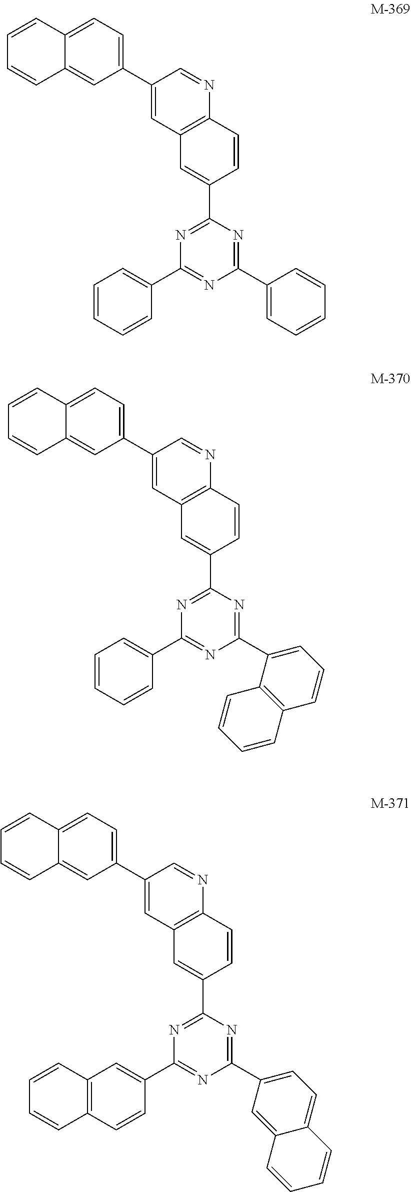 [SMILES: c1ccc(-c2nc(-c3ccccc3)nc(-c3ccc4ncc(-c5ccc6ccccc6c5)cc4c3)n2)cc1 ; c1ccc(-c2nc(-c3ccc4ncc(-c5ccc6ccccc6c5)cc4c3)nc(-c3cccc4ccccc34)n2)cc1 ; c1ccc2cc(-c3cnc4ccc(-c5nc(-c6ccc7ccccc7c6)nc(-c6ccc7ccccc7c6)n5)cc4c3)ccc2c1]

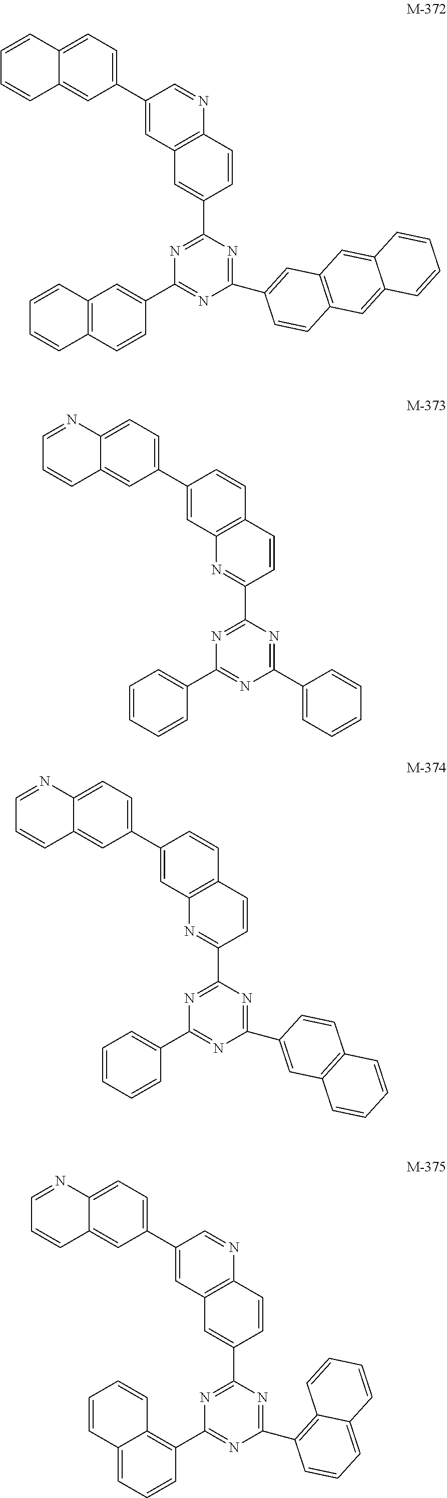 [SMILES: c1ccc2cc(-c3cnc4ccc(-c5nc(-c6ccc7ccccc7c6)nc(-c6ccc7cc8ccccc8cc7c6)n5)cc4c3)ccc2c1 ; c1ccc(-c2nc(-c3ccccc3)nc(-c3ccc4ccc(-c5ccc6ncccc6c5)cc4n3)n2)cc1 ; c1ccc(-c2nc(-c3ccc4ccccc4c3)nc(-c3ccc4ccc(-c5ccc6ncccc6c5)cc4n3)n2)cc1 ; c1cnc2ccc(-c3cnc4ccc(-c5nc(-c6cccc7ccccc67)nc(-c6cccc7ccccc67)n5)cc4c3)cc2c1]

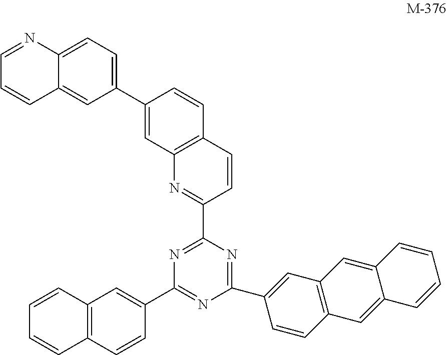 [SMILES: c1ccc2cc(-c3nc(-c4ccc5cc6ccccc6cc5c4)nc(-c4ccc5ccc(-c6ccc7ncccc7c6)cc5n4)n3)ccc2c1]

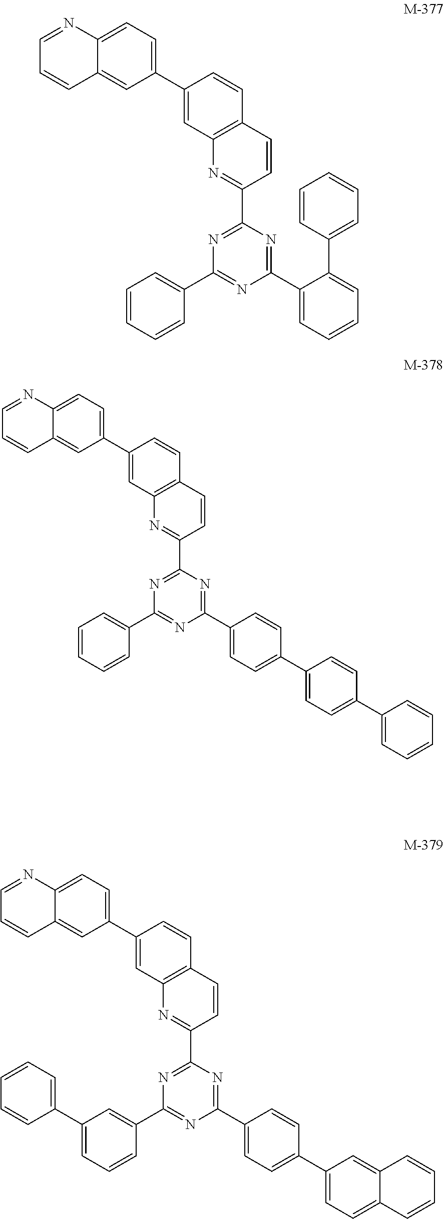 [SMILES: c1ccc(-c2nc(-c3ccc4ccc(-c5ccc6ncccc6c5)cc4n3)nc(-c3ccccc3-c3ccccc3)n2)cc1 ; c1ccc(-c2ccc(-c3ccc(-c4nc(-c5ccccc5)nc(-c5ccc6ccc(-c7ccc8ncccc8c7)cc6n5)n4)cc3)cc2)cc1 ; c1ccc(-c2cccc(-c3nc(-c4ccc(-c5ccc6ccccc6c5)cc4)nc(-c4ccc5ccc(-c6ccc7ncccc7c6)cc5n4)n3)c2)cc1]

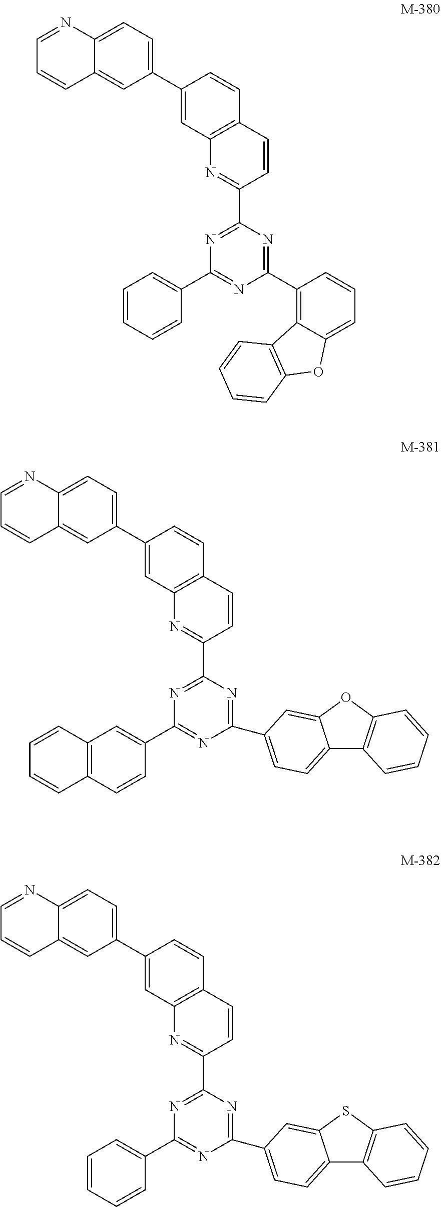 [SMILES: c1ccc(-c2nc(-c3ccc4ccc(-c5ccc6ncccc6c5)cc4n3)nc(-c3cccc4oc5ccccc5c34)n2)cc1 ; c1ccc2cc(-c3nc(-c4ccc5c(c4)oc4ccccc45)nc(-c4ccc5ccc(-c6ccc7ncccc7c6)cc5n4)n3)ccc2c1 ; c1ccc(-c2nc(-c3ccc4c(c3)sc3ccccc34)nc(-c3ccc4ccc(-c5ccc6ncccc6c5)cc4n3)n2)cc1]

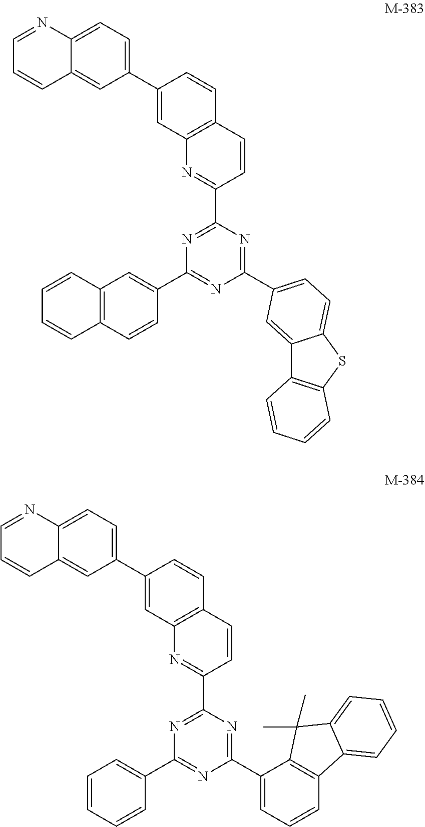 [SMILES: c1ccc2cc(-c3nc(-c4ccc5sc6ccccc6c5c4)nc(-c4ccc5ccc(-c6ccc7ncccc7c6)cc5n4)n3)ccc2c1 ; CC1(C)c2ccccc2-c2cccc(-c3nc(-c4ccccc4)nc(-c4ccc5ccc(-c6ccc7ncccc7c6)cc5n4)n3)c21]

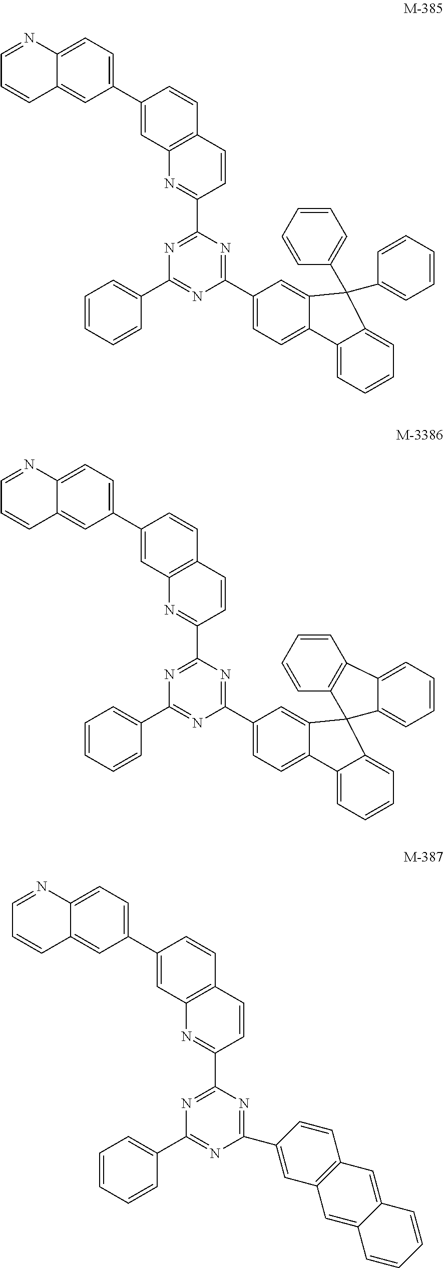 [SMILES: c1ccc(-c2nc(-c3ccc4c(c3)C(c3ccccc3)(c3ccccc3)c3ccccc3-4)nc(-c3ccc4ccc(-c5ccc6ncccc6c5)cc4n3)n2)cc1 ; c1ccc(-c2nc(-c3ccc4c(c3)C3(c5ccccc5-c5ccccc53)c3ccccc3-4)nc(-c3ccc4ccc(-c5ccc6ncccc6c5)cc4n3)n2)cc1 ; c1ccc(-c2nc(-c3ccc4cc5ccccc5cc4c3)nc(-c3ccc4ccc(-c5ccc6ncccc6c5)cc4n3)n2)cc1]

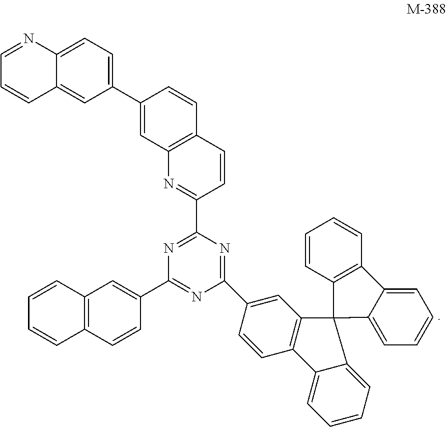 [SMILES: c1ccc2c(c1)-c1ccccc1C21c2ccccc2-c2ccc(-c3nc(-c4ccc5ccccc5c4)nc(-c4ccc5ccc(-c6ccc7ncccc7c6)cc5n4)n3)cc21]

The structure of the compound M is as shown in any one of M-389 to M-619 below.

The structures of M-389 to M-619 are as follows:

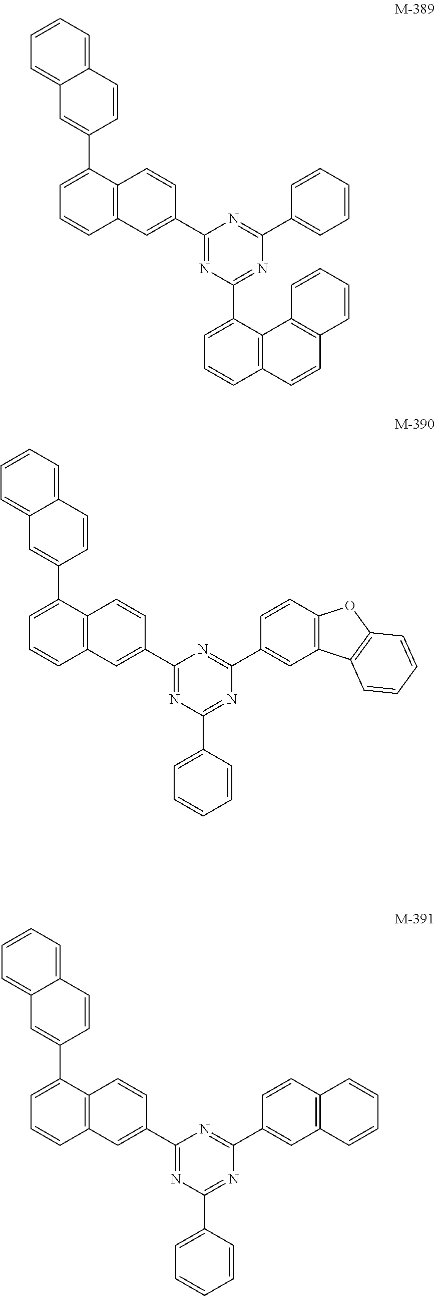 [SMILES: c1ccc(-c2nc(-c3ccc4c(-c5ccc6ccccc6c5)cccc4c3)nc(-c3cccc4ccc5ccccc5c34)n2)cc1 ; c1ccc(-c2nc(-c3ccc4c(-c5ccc6ccccc6c5)cccc4c3)nc(-c3ccc4oc5ccccc5c4c3)n2)cc1 ; c1ccc(-c2nc(-c3ccc4ccccc4c3)nc(-c3ccc4c(-c5ccc6ccccc6c5)cccc4c3)n2)cc1]

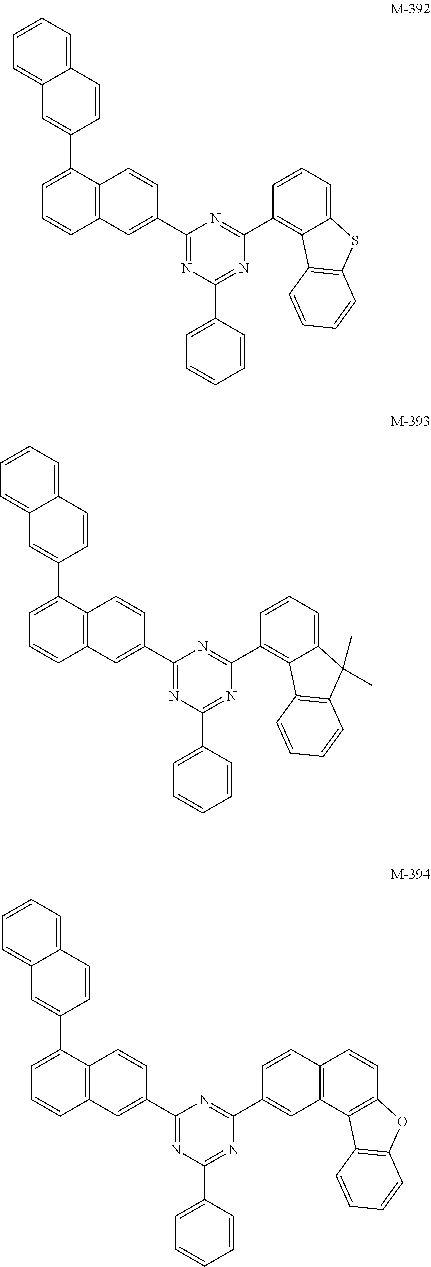 [SMILES: c1ccc(-c2nc(-c3ccc4c(-c5ccc6ccccc6c5)cccc4c3)nc(-c3cccc4sc5ccccc5c34)n2)cc1 ; CC1(C)c2ccccc2-c2c(-c3nc(-c4ccccc4)nc(-c4ccc5c(-c6ccc7ccccc7c6)cccc5c4)n3)cccc21 ; c1ccc(-c2nc(-c3ccc4c(-c5ccc6ccccc6c5)cccc4c3)nc(-c3ccc4ccc5oc6ccccc6c5c4c3)n2)cc1]

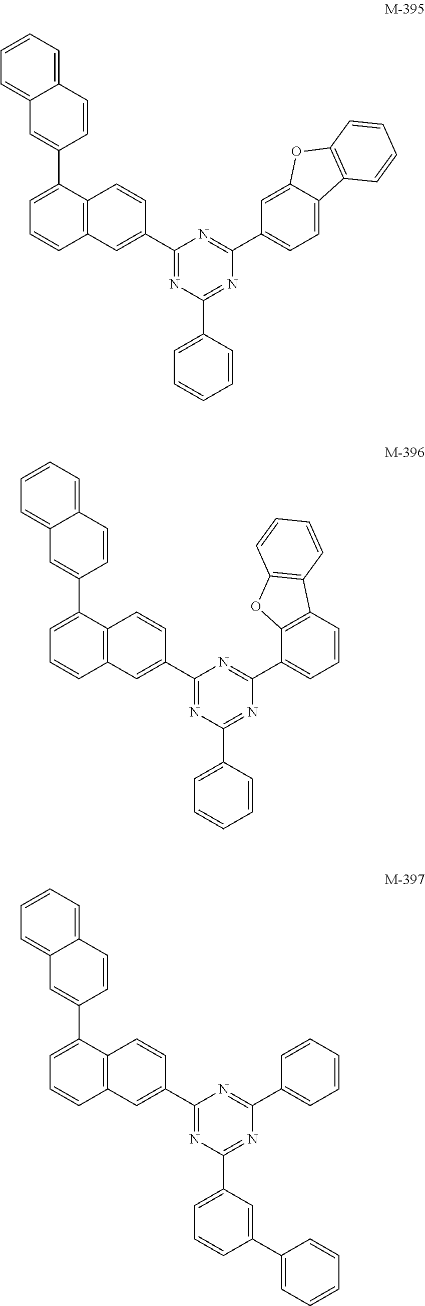 [SMILES: c1ccc(-c2nc(-c3ccc4c(-c5ccc6ccccc6c5)cccc4c3)nc(-c3ccc4c(c3)oc3ccccc34)n2)cc1 ; c1ccc(-c2nc(-c3ccc4c(-c5ccc6ccccc6c5)cccc4c3)nc(-c3cccc4c3oc3ccccc34)n2)cc1 ; c1ccc(-c2cccc(-c3nc(-c4ccccc4)nc(-c4ccc5c(-c6ccc7ccccc7c6)cccc5c4)n3)c2)cc1]

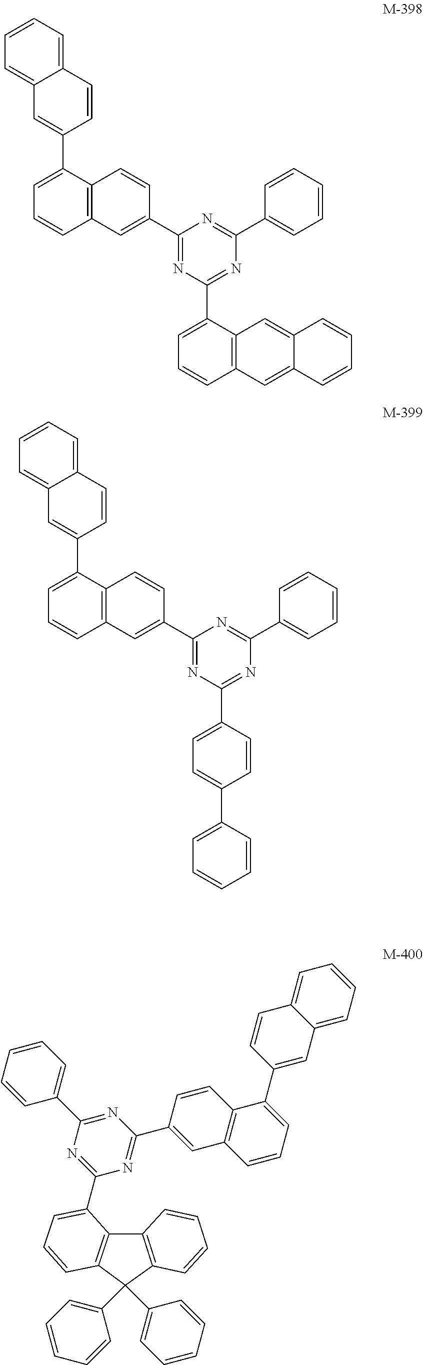 [SMILES: c1ccc(-c2nc(-c3ccc4c(-c5ccc6ccccc6c5)cccc4c3)nc(-c3cccc4cc5ccccc5cc34)n2)cc1 ; c1ccc(-c2ccc(-c3nc(-c4ccccc4)nc(-c4ccc5c(-c6ccc7ccccc7c6)cccc5c4)n3)cc2)cc1 ; c1ccc(-c2nc(-c3ccc4c(-c5ccc6ccccc6c5)cccc4c3)nc(-c3cccc4c3-c3ccccc3C4(c3ccccc3)c3ccccc3)n2)cc1]

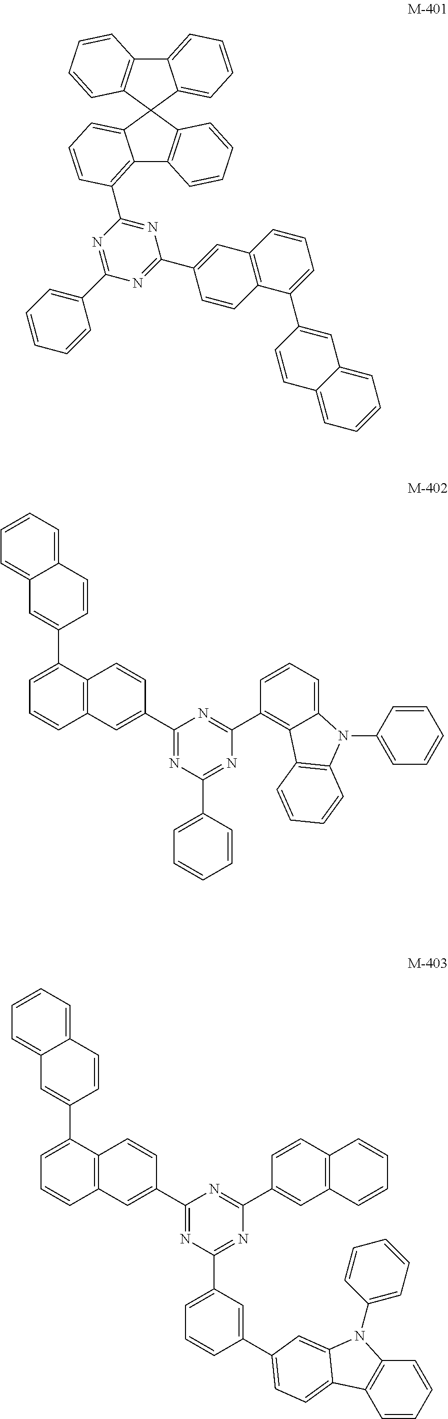 [SMILES: c1ccc(-c2nc(-c3ccc4c(-c5ccc6ccccc6c5)cccc4c3)nc(-c3cccc4c3-c3ccccc3C43c4ccccc4-c4ccccc43)n2)cc1 ; c1ccc(-c2nc(-c3ccc4c(-c5ccc6ccccc6c5)cccc4c3)nc(-c3cccc4c3c3ccccc3n4-c3ccccc3)n2)cc1 ; c1ccc(-n2c3ccccc3c3ccc(-c4cccc(-c5nc(-c6ccc7ccccc7c6)nc(-c6ccc7c(-c8ccc9ccccc9c8)cccc7c6)n5)c4)cc32)cc1]

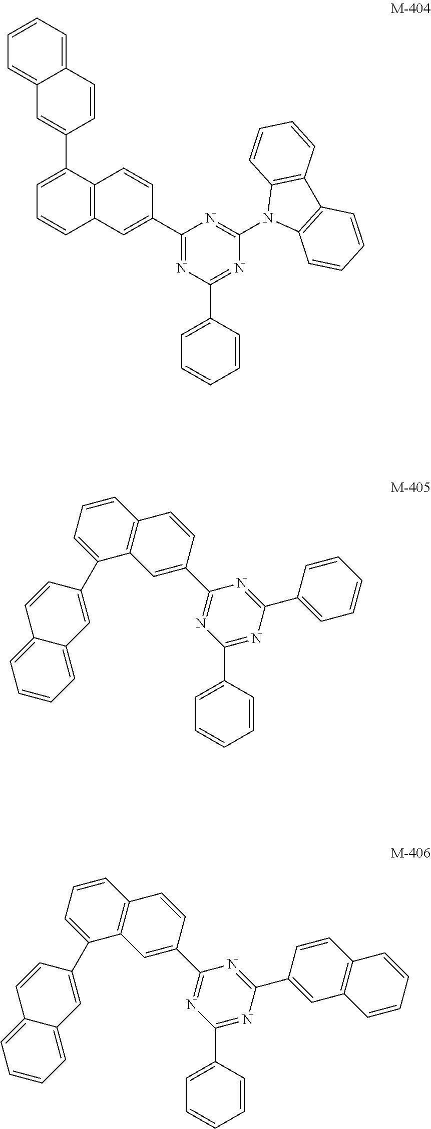 [SMILES: c1ccc(-c2nc(-c3ccc4c(-c5ccc6ccccc6c5)cccc4c3)nc(-n3c4ccccc4c4ccccc43)n2)cc1 ; c1ccc(-c2nc(-c3ccccc3)nc(-c3ccc4cccc(-c5ccc6ccccc6c5)c4c3)n2)cc1 ; c1ccc(-c2nc(-c3ccc4ccccc4c3)nc(-c3ccc4cccc(-c5ccc6ccccc6c5)c4c3)n2)cc1]

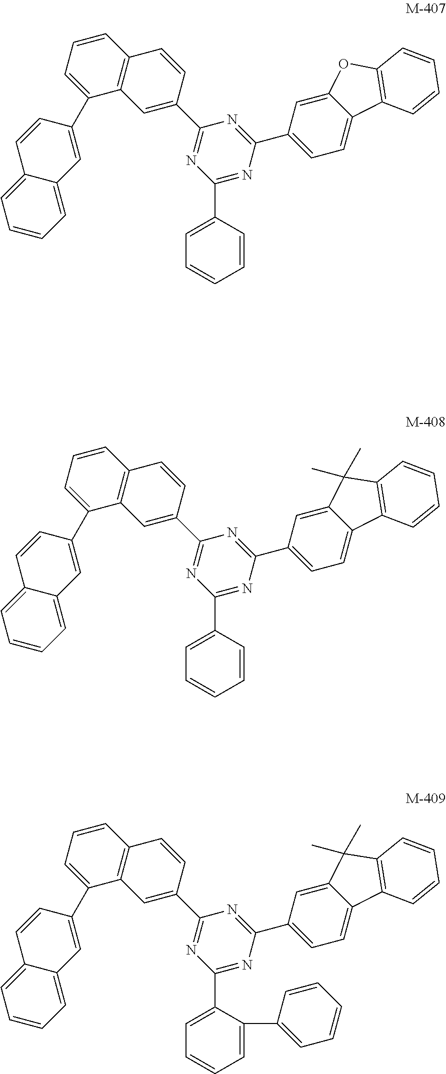 [SMILES: c1ccc(-c2nc(-c3ccc4c(c3)oc3ccccc34)nc(-c3ccc4cccc(-c5ccc6ccccc6c5)c4c3)n2)cc1 ; CC1(C)c2ccccc2-c2ccc(-c3nc(-c4ccccc4)nc(-c4ccc5cccc(-c6ccc7ccccc7c6)c5c4)n3)cc21 ; CC1(C)c2ccccc2-c2ccc(-c3nc(-c4ccc5cccc(-c6ccc7ccccc7c6)c5c4)nc(-c4ccccc4-c4ccccc4)n3)cc21]

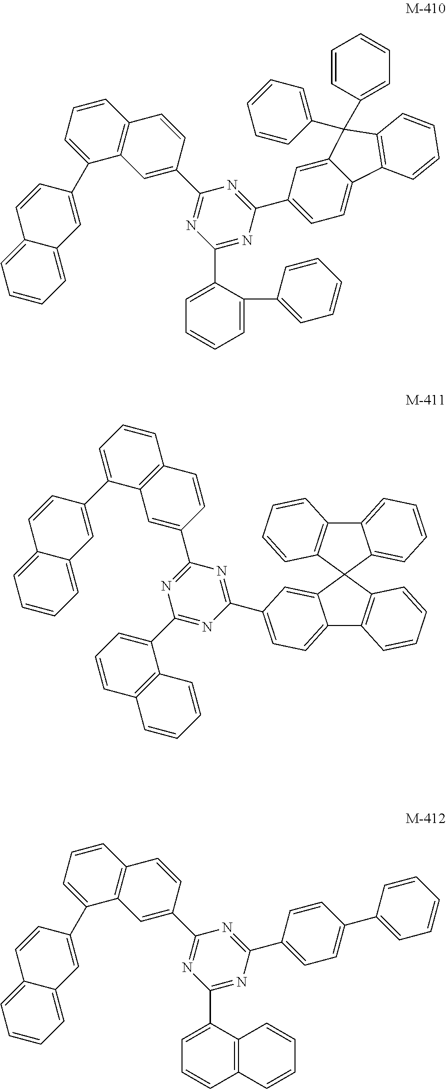 [SMILES: c1ccc(-c2ccccc2-c2nc(-c3ccc4c(c3)C(c3ccccc3)(c3ccccc3)c3ccccc3-4)nc(-c3ccc4cccc(-c5ccc6ccccc6c5)c4c3)n2)cc1 ; c1ccc2c(c1)-c1ccccc1C21c2ccccc2-c2ccc(-c3nc(-c4ccc5cccc(-c6ccc7ccccc7c6)c5c4)nc(-c4cccc5ccccc45)n3)cc21 ; c1ccc(-c2ccc(-c3nc(-c4ccc5cccc(-c6ccc7ccccc7c6)c5c4)nc(-c4cccc5ccccc45)n3)cc2)cc1]

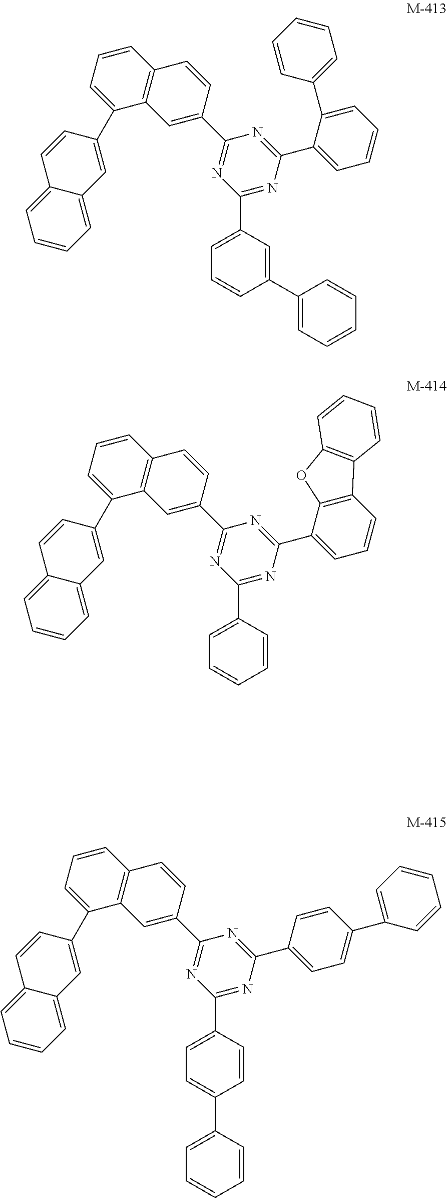 [SMILES: c1ccc(-c2cccc(-c3nc(-c4ccc5cccc(-c6ccc7ccccc7c6)c5c4)nc(-c4ccccc4-c4ccccc4)n3)c2)cc1 ; c1ccc(-c2nc(-c3ccc4cccc(-c5ccc6ccccc6c5)c4c3)nc(-c3cccc4c3oc3ccccc34)n2)cc1 ; c1ccc(-c2ccc(-c3nc(-c4ccc(-c5ccccc5)cc4)nc(-c4ccc5cccc(-c6ccc7ccccc7c6)c5c4)n3)cc2)cc1]

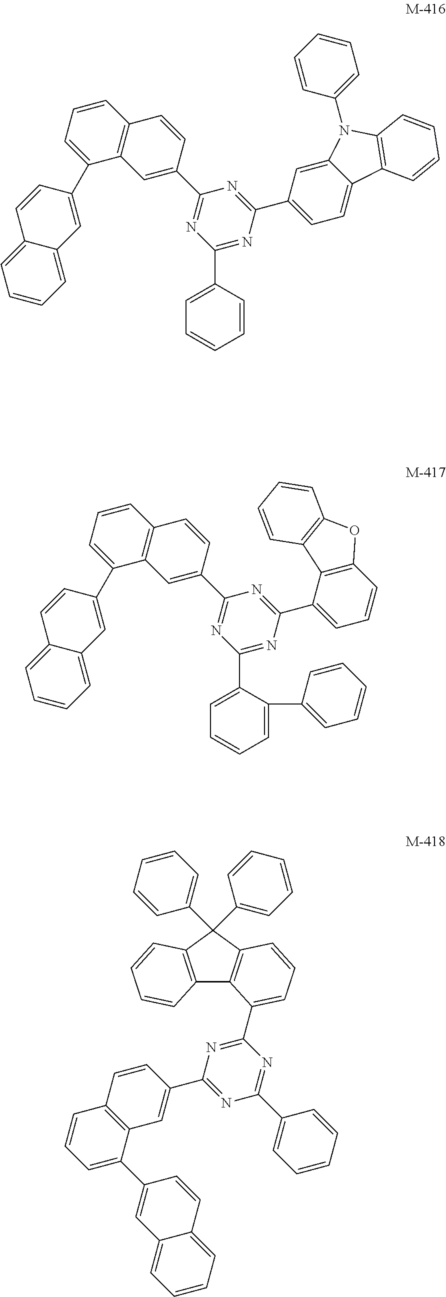 [SMILES: c1ccc(-c2nc(-c3ccc4cccc(-c5ccc6ccccc6c5)c4c3)nc(-c3ccc4c5ccccc5n(-c5ccccc5)c4c3)n2)cc1 ; c1ccc(-c2ccccc2-c2nc(-c3ccc4cccc(-c5ccc6ccccc6c5)c4c3)nc(-c3cccc4oc5ccccc5c34)n2)cc1 ; c1ccc(-c2nc(-c3ccc4cccc(-c5ccc6ccccc6c5)c4c3)nc(-c3cccc4c3-c3ccccc3C4(c3ccccc3)c3ccccc3)n2)cc1]

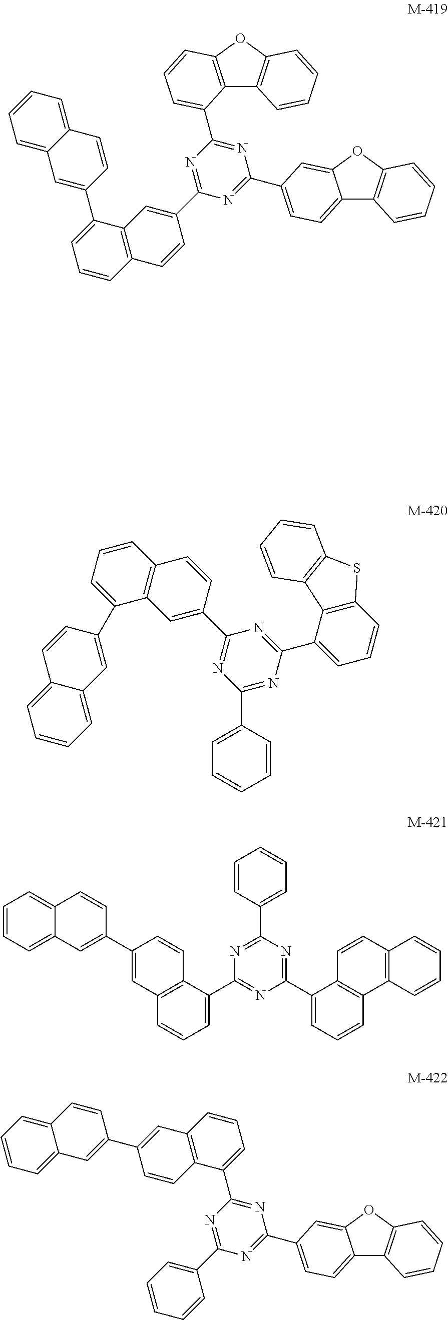 [SMILES: c1ccc2cc(-c3cccc4ccc(-c5nc(-c6ccc7c(c6)oc6ccccc67)nc(-c6cccc7oc8ccccc8c67)n5)cc34)ccc2c1 ; c1ccc(-c2nc(-c3ccc4cccc(-c5ccc6ccccc6c5)c4c3)nc(-c3cccc4sc5ccccc5c34)n2)cc1 ; c1ccc(-c2nc(-c3cccc4cc(-c5ccc6ccccc6c5)ccc34)nc(-c3cccc4c3ccc3ccccc34)n2)cc1 ; c1ccc(-c2nc(-c3ccc4c(c3)oc3ccccc34)nc(-c3cccc4cc(-c5ccc6ccccc6c5)ccc34)n2)cc1]

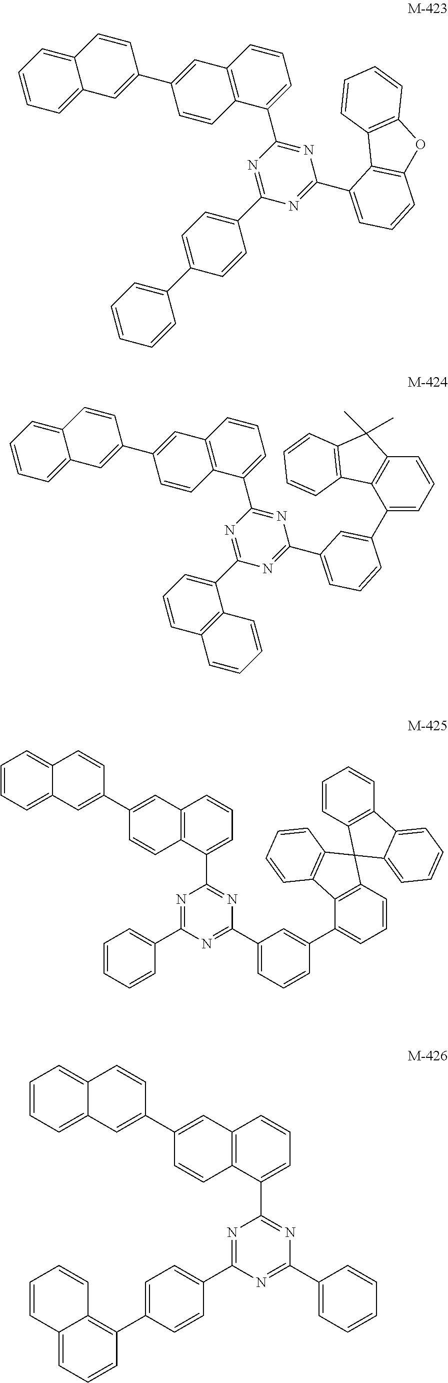 [SMILES: c1ccc(-c2ccc(-c3nc(-c4cccc5cc(-c6ccc7ccccc7c6)ccc45)nc(-c4cccc5oc6ccccc6c45)n3)cc2)cc1 ; CC1(C)c2ccccc2-c2c(-c3cccc(-c4nc(-c5cccc6ccccc56)nc(-c5cccc6cc(-c7ccc8ccccc8c7)ccc56)n4)c3)cccc21 ; c1ccc(-c2nc(-c3cccc(-c4cccc5c4-c4ccccc4C54c5ccccc5-c5ccccc54)c3)nc(-c3cccc4cc(-c5ccc6ccccc6c5)ccc34)n2)cc1 ; c1ccc(-c2nc(-c3ccc(-c4cccc5ccccc45)cc3)nc(-c3cccc4cc(-c5ccc6ccccc6c5)ccc34)n2)cc1]

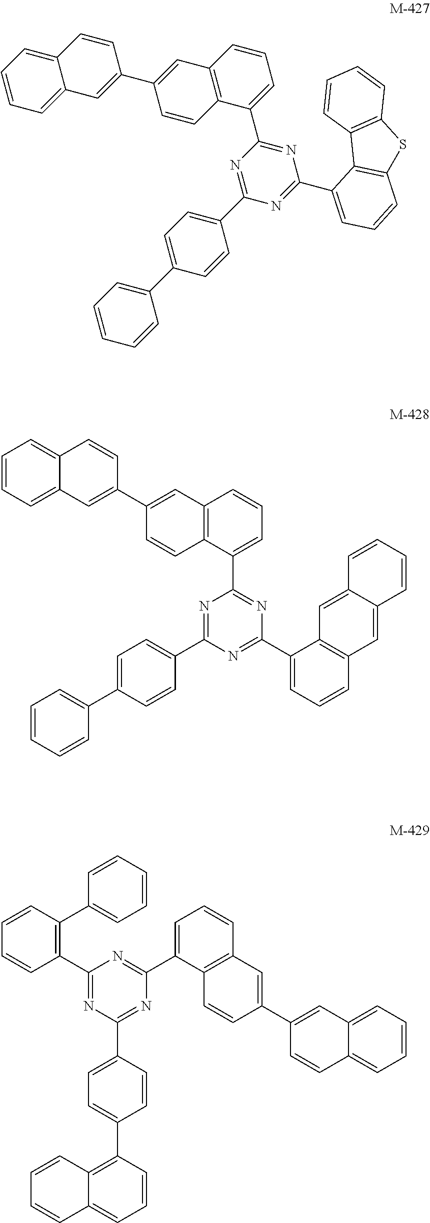 [SMILES: c1ccc(-c2ccc(-c3nc(-c4cccc5cc(-c6ccc7ccccc7c6)ccc45)nc(-c4cccc5sc6ccccc6c45)n3)cc2)cc1 ; c1ccc(-c2ccc(-c3nc(-c4cccc5cc(-c6ccc7ccccc7c6)ccc45)nc(-c4cccc5cc6ccccc6cc45)n3)cc2)cc1 ; c1ccc(-c2ccccc2-c2nc(-c3ccc(-c4cccc5ccccc45)cc3)nc(-c3cccc4cc(-c5ccc6ccccc6c5)ccc34)n2)cc1]

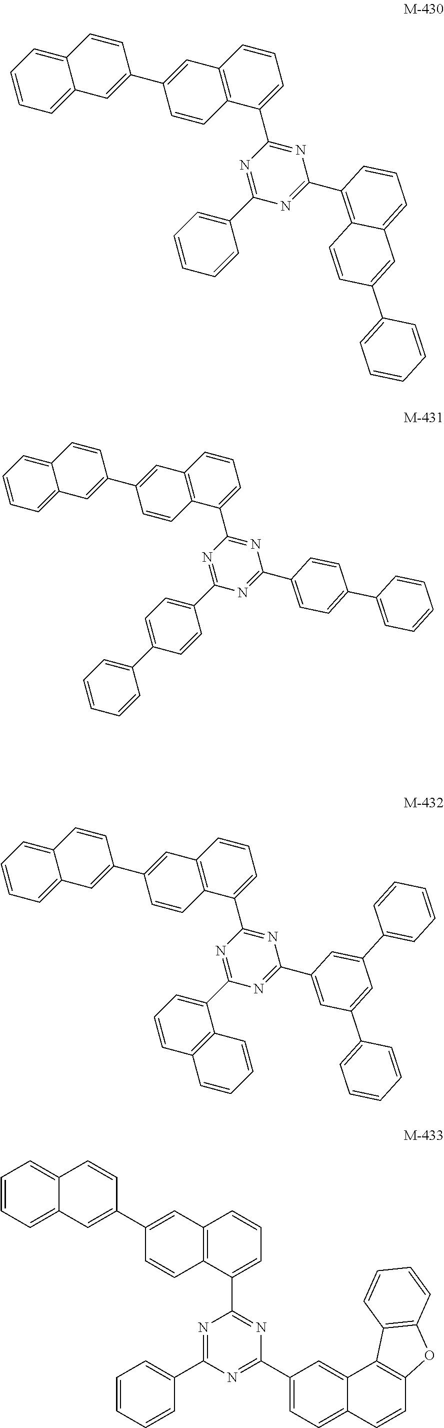 [SMILES: c1ccc(-c2ccc3c(-c4nc(-c5ccccc5)nc(-c5cccc6cc(-c7ccc8ccccc8c7)ccc56)n4)cccc3c2)cc1 ; c1ccc(-c2ccc(-c3nc(-c4ccc(-c5ccccc5)cc4)nc(-c4cccc5cc(-c6ccc7ccccc7c6)ccc45)n3)cc2)cc1 ; c1ccc(-c2cc(-c3ccccc3)cc(-c3nc(-c4cccc5ccccc45)nc(-c4cccc5cc(-c6ccc7ccccc7c6)ccc45)n3)c2)cc1 ; c1ccc(-c2nc(-c3ccc4ccc5oc6ccccc6c5c4c3)nc(-c3cccc4cc(-c5ccc6ccccc6c5)ccc34)n2)cc1]

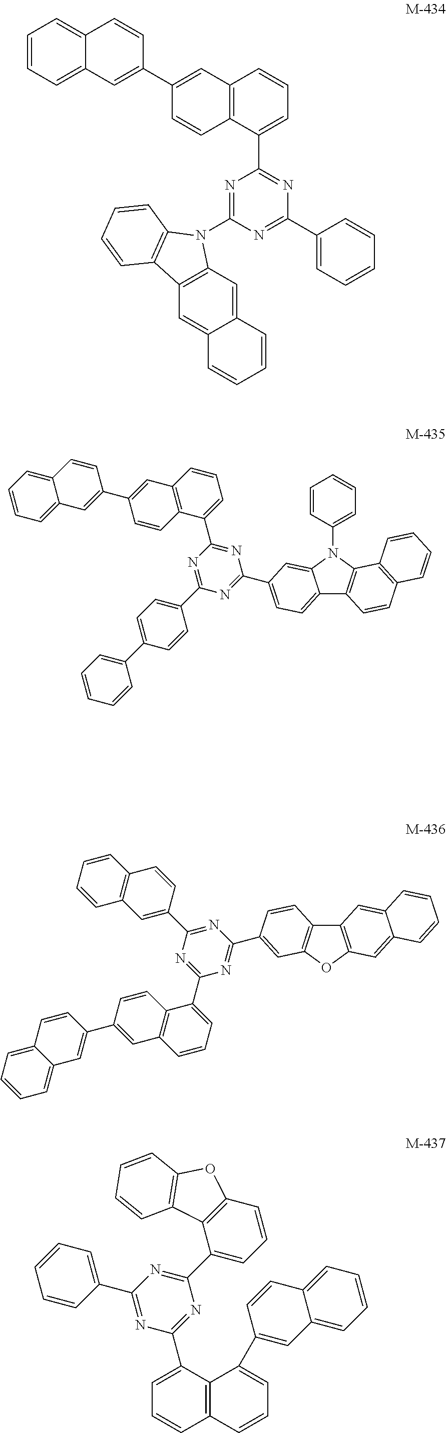 [SMILES: c1ccc(-c2nc(-c3cccc4cc(-c5ccc6ccccc6c5)ccc34)nc(-n3c4ccccc4c4cc5ccccc5cc43)n2)cc1 ; c1ccc(-c2ccc(-c3nc(-c4ccc5c6ccc7ccccc7c6n(-c6ccccc6)c5c4)nc(-c4cccc5cc(-c6ccc7ccccc7c6)ccc45)n3)cc2)cc1 ; c1ccc2cc(-c3ccc4c(-c5nc(-c6ccc7ccccc7c6)nc(-c6ccc7c(c6)oc6cc8ccccc8cc67)n5)cccc4c3)ccc2c1 ; c1ccc(-c2nc(-c3cccc4cccc(-c5ccc6ccccc6c5)c34)nc(-c3cccc4oc5ccccc5c34)n2)cc1]

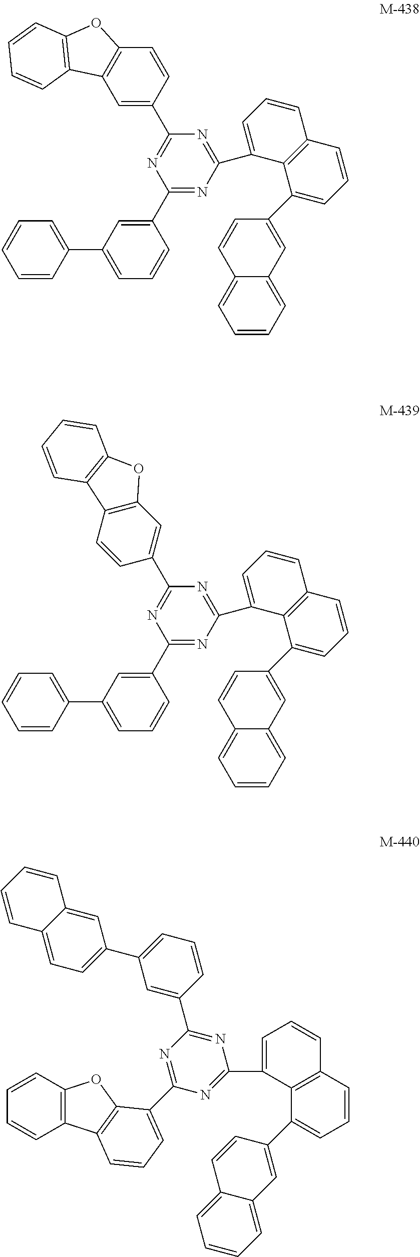 [SMILES: c1ccc(-c2cccc(-c3nc(-c4ccc5oc6ccccc6c5c4)nc(-c4cccc5cccc(-c6ccc7ccccc7c6)c45)n3)c2)cc1 ; c1ccc(-c2cccc(-c3nc(-c4ccc5c(c4)oc4ccccc45)nc(-c4cccc5cccc(-c6ccc7ccccc7c6)c45)n3)c2)cc1 ; c1cc(-c2ccc3ccccc3c2)cc(-c2nc(-c3cccc4c3oc3ccccc34)nc(-c3cccc4cccc(-c5ccc6ccccc6c5)c34)n2)c1]

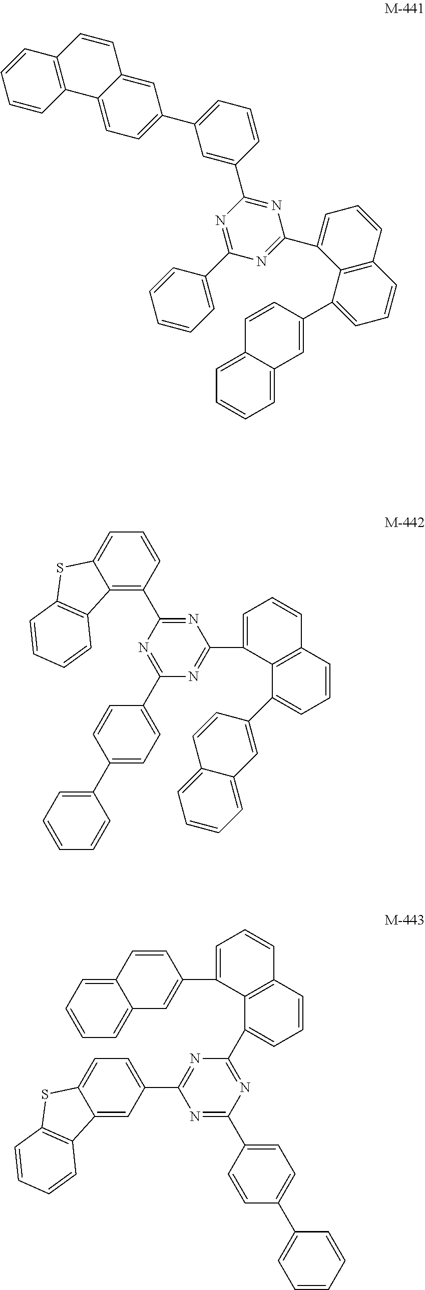 [SMILES: c1ccc(-c2nc(-c3cccc(-c4ccc5c(ccc6ccccc65)c4)c3)nc(-c3cccc4cccc(-c5ccc6ccccc6c5)c34)n2)cc1 ; c1ccc(-c2ccc(-c3nc(-c4cccc5cccc(-c6ccc7ccccc7c6)c45)nc(-c4cccc5sc6ccccc6c45)n3)cc2)cc1 ; c1ccc(-c2ccc(-c3nc(-c4ccc5sc6ccccc6c5c4)nc(-c4cccc5cccc(-c6ccc7ccccc7c6)c45)n3)cc2)cc1]

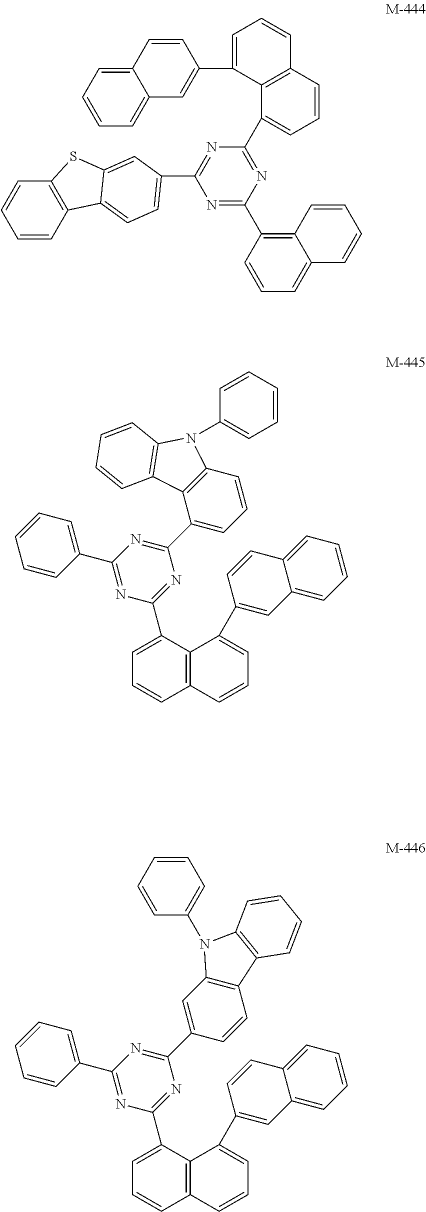 [SMILES: c1ccc2cc(-c3cccc4cccc(-c5nc(-c6ccc7c(c6)sc6ccccc67)nc(-c6cccc7ccccc67)n5)c34)ccc2c1 ; c1ccc(-c2nc(-c3cccc4cccc(-c5ccc6ccccc6c5)c34)nc(-c3cccc4c3c3ccccc3n4-c3ccccc3)n2)cc1 ; c1ccc(-c2nc(-c3ccc4c5ccccc5n(-c5ccccc5)c4c3)nc(-c3cccc4cccc(-c5ccc6ccccc6c5)c34)n2)cc1]

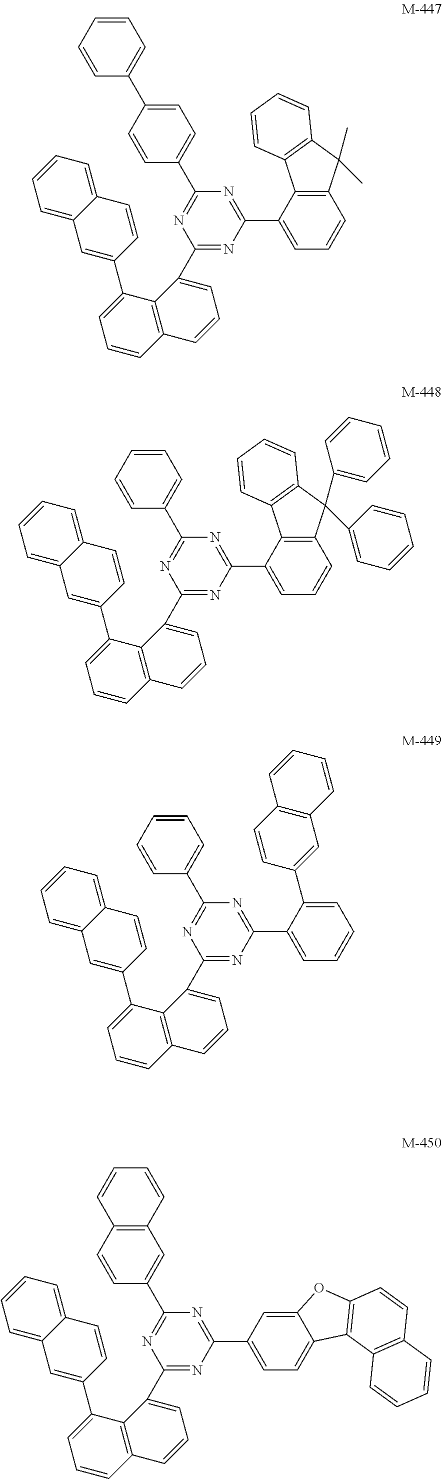 [SMILES: CC1(C)c2ccccc2-c2c(-c3nc(-c4ccc(-c5ccccc5)cc4)nc(-c4cccc5cccc(-c6ccc7ccccc7c6)c45)n3)cccc21 ; c1ccc(-c2nc(-c3cccc4c3-c3ccccc3C4(c3ccccc3)c3ccccc3)nc(-c3cccc4cccc(-c5ccc6ccccc6c5)c34)n2)cc1 ; c1ccc(-c2nc(-c3ccccc3-c3ccc4ccccc4c3)nc(-c3cccc4cccc(-c5ccc6ccccc6c5)c34)n2)cc1 ; c1ccc2cc(-c3nc(-c4ccc5c(c4)oc4ccc6ccccc6c45)nc(-c4cccc5cccc(-c6ccc7ccccc7c6)c45)n3)ccc2c1]

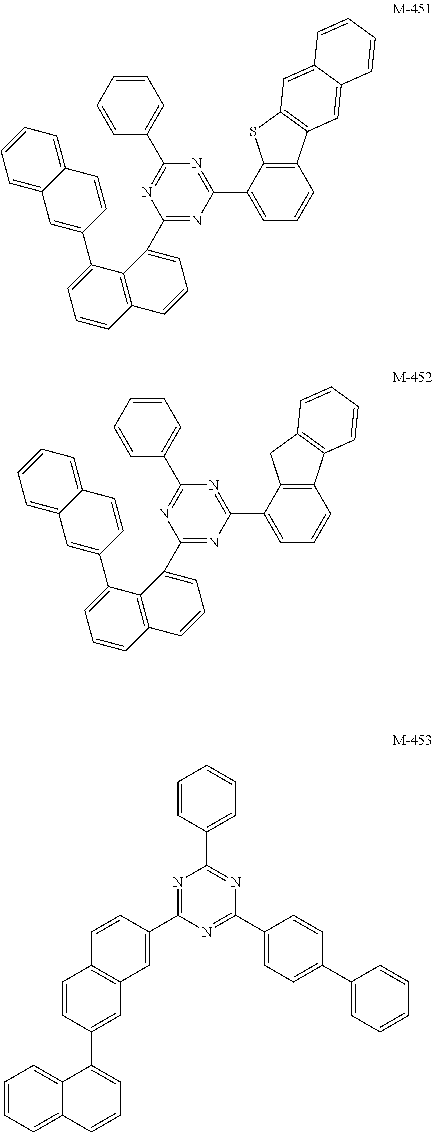 [SMILES: c1ccc(-c2nc(-c3cccc4c3sc3cc5ccccc5cc34)nc(-c3cccc4cccc(-c5ccc6ccccc6c5)c34)n2)cc1 ; c1ccc(-c2nc(-c3cccc4c3Cc3ccccc3-4)nc(-c3cccc4cccc(-c5ccc6ccccc6c5)c34)n2)cc1 ; c1ccc(-c2ccc(-c3nc(-c4ccccc4)nc(-c4ccc5ccc(-c6cccc7ccccc67)cc5c4)n3)cc2)cc1]

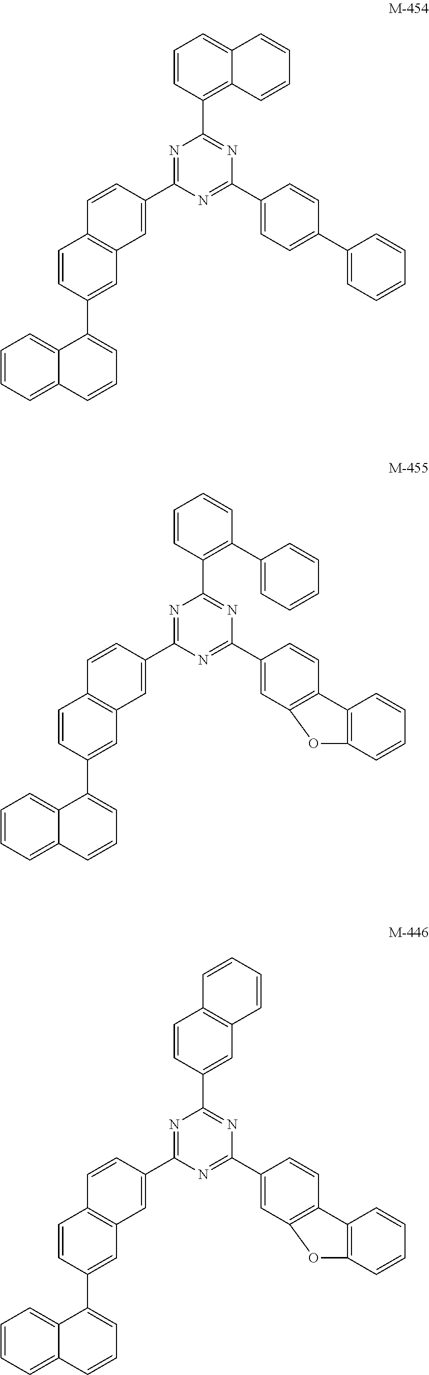 [SMILES: c1ccc(-c2ccc(-c3nc(-c4ccc5ccc(-c6cccc7ccccc67)cc5c4)nc(-c4cccc5ccccc45)n3)cc2)cc1 ; c1ccc(-c2ccccc2-c2nc(-c3ccc4ccc(-c5cccc6ccccc56)cc4c3)nc(-c3ccc4c(c3)oc3ccccc34)n2)cc1 ; c1ccc2cc(-c3nc(-c4ccc5ccc(-c6cccc7ccccc67)cc5c4)nc(-c4ccc5c(c4)oc4ccccc45)n3)ccc2c1]

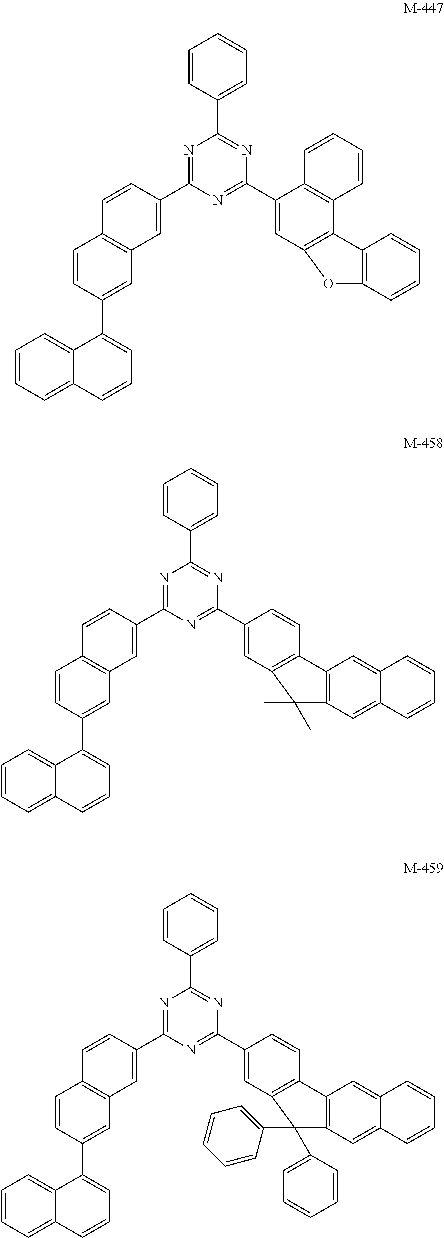 [SMILES: c1ccc(-c2nc(-c3ccc4ccc(-c5cccc6ccccc56)cc4c3)nc(-c3cc4oc5ccccc5c4c4ccccc34)n2)cc1 ; CC1(C)c2cc(-c3nc(-c4ccccc4)nc(-c4ccc5ccc(-c6cccc7ccccc67)cc5c4)n3)ccc2-c2cc3ccccc3cc21 ; c1ccc(-c2nc(-c3ccc4c(c3)C(c3ccccc3)(c3ccccc3)c3cc5ccccc5cc3-4)nc(-c3ccc4ccc(-c5cccc6ccccc56)cc4c3)n2)cc1]

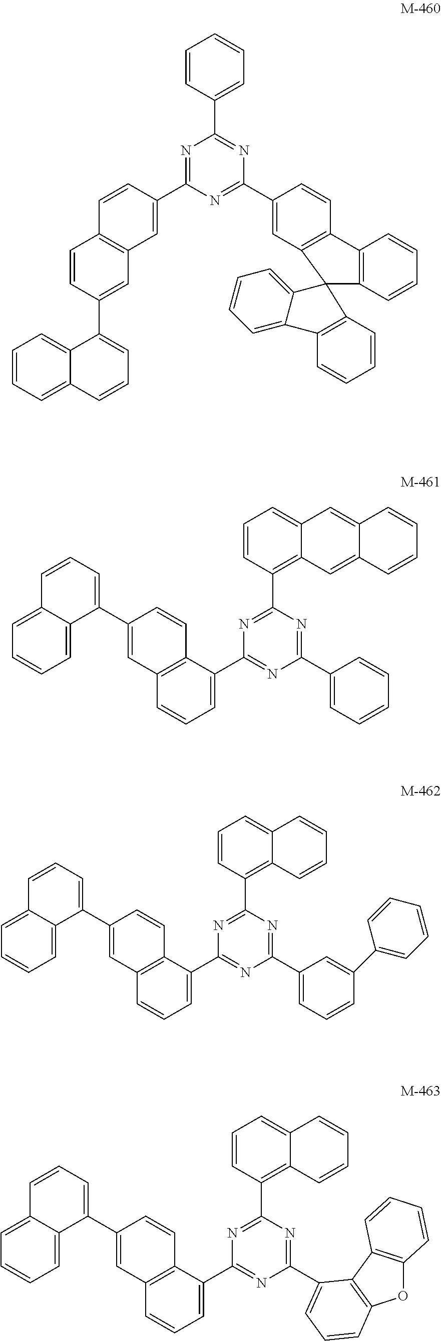 [SMILES: c1ccc(-c2nc(-c3ccc4c(c3)C3(c5ccccc5-c5ccccc53)c3ccccc3-4)nc(-c3ccc4ccc(-c5cccc6ccccc56)cc4c3)n2)cc1 ; c1ccc(-c2nc(-c3cccc4cc(-c5cccc6ccccc56)ccc34)nc(-c3cccc4cc5ccccc5cc34)n2)cc1 ; c1ccc(-c2cccc(-c3nc(-c4cccc5ccccc45)nc(-c4cccc5cc(-c6cccc7ccccc67)ccc45)n3)c2)cc1 ; c1ccc2c(-c3ccc4c(-c5nc(-c6cccc7ccccc67)nc(-c6cccc7oc8ccccc8c67)n5)cccc4c3)cccc2c1]

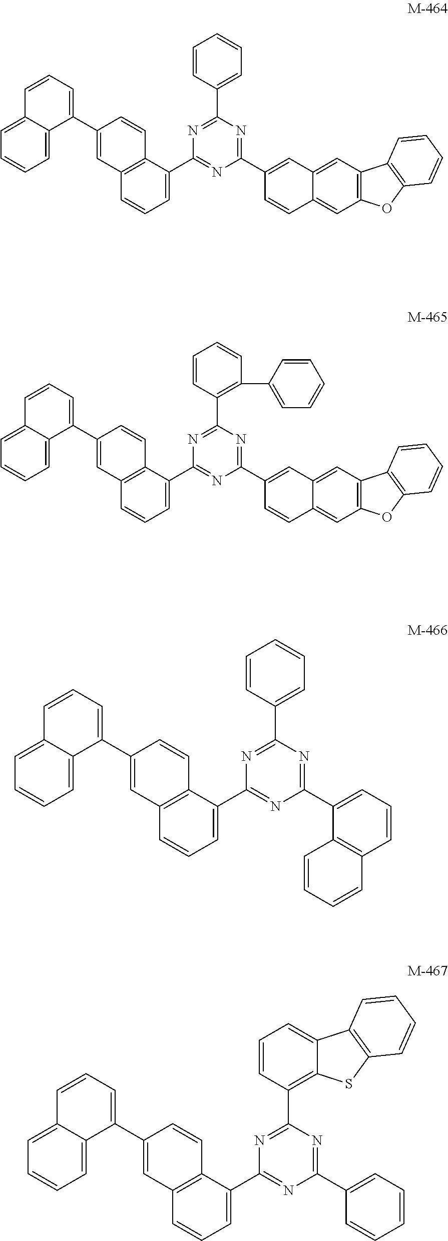 [SMILES: c1ccc(-c2nc(-c3ccc4cc5oc6ccccc6c5cc4c3)nc(-c3cccc4cc(-c5cccc6ccccc56)ccc34)n2)cc1 ; c1ccc(-c2ccccc2-c2nc(-c3ccc4cc5oc6ccccc6c5cc4c3)nc(-c3cccc4cc(-c5cccc6ccccc56)ccc34)n2)cc1 ; c1ccc(-c2nc(-c3cccc4ccccc34)nc(-c3cccc4cc(-c5cccc6ccccc56)ccc34)n2)cc1 ; c1ccc(-c2nc(-c3cccc4cc(-c5cccc6ccccc56)ccc34)nc(-c3cccc4c3sc3ccccc34)n2)cc1]

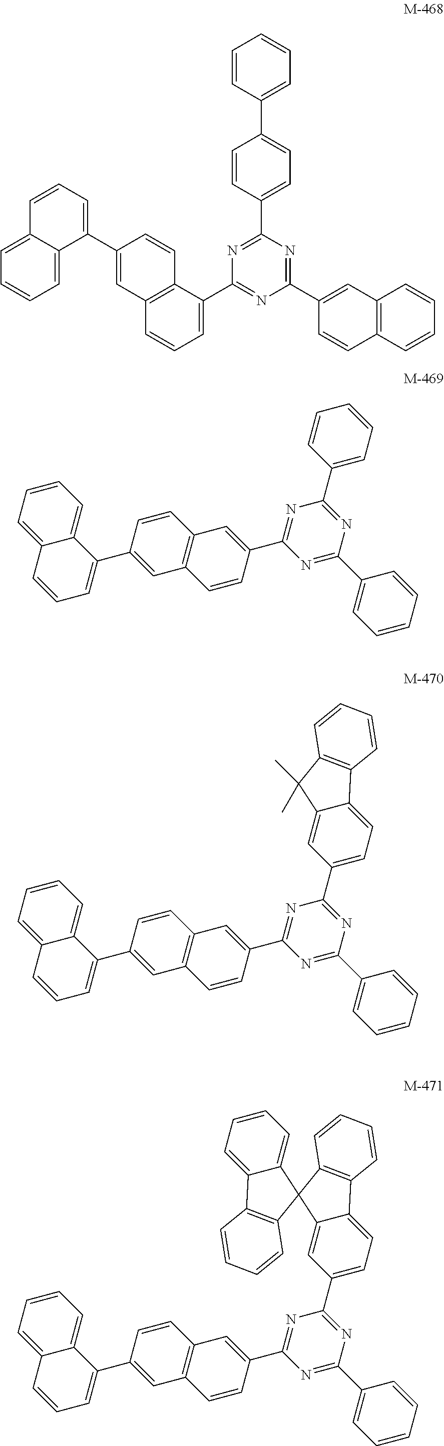 [SMILES: c1ccc(-c2ccc(-c3nc(-c4ccc5ccccc5c4)nc(-c4cccc5cc(-c6cccc7ccccc67)ccc45)n3)cc2)cc1 ; c1ccc(-c2nc(-c3ccccc3)nc(-c3ccc4cc(-c5cccc6ccccc56)ccc4c3)n2)cc1 ; CC1(C)c2ccccc2-c2ccc(-c3nc(-c4ccccc4)nc(-c4ccc5cc(-c6cccc7ccccc67)ccc5c4)n3)cc21 ; c1ccc(-c2nc(-c3ccc4c(c3)C3(c5ccccc5-c5ccccc53)c3ccccc3-4)nc(-c3ccc4cc(-c5cccc6ccccc56)ccc4c3)n2)cc1]

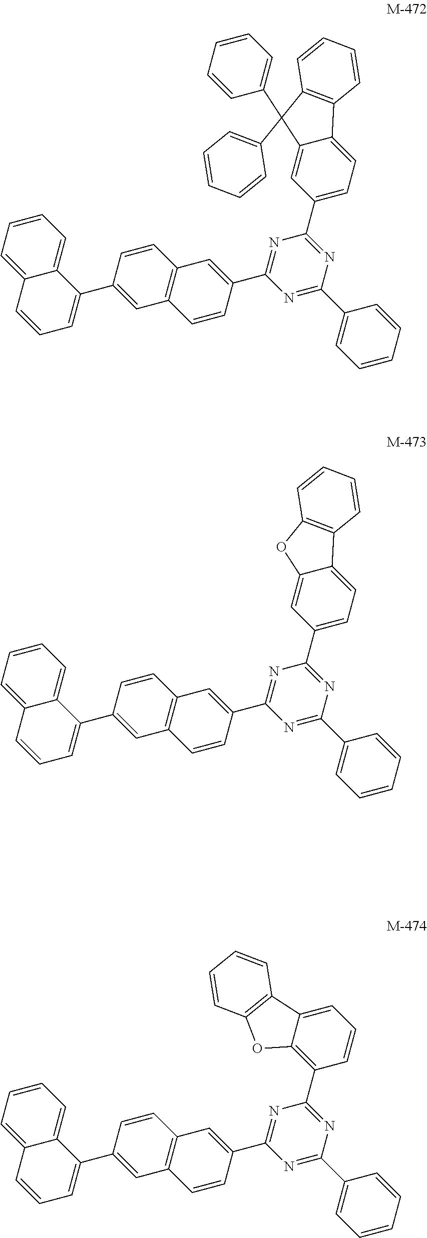 [SMILES: c1ccc(-c2nc(-c3ccc4c(c3)C(c3ccccc3)(c3ccccc3)c3ccccc3-4)nc(-c3ccc4cc(-c5cccc6ccccc56)ccc4c3)n2)cc1 ; c1ccc(-c2nc(-c3ccc4cc(-c5cccc6ccccc56)ccc4c3)nc(-c3ccc4c(c3)oc3ccccc34)n2)cc1 ; c1ccc(-c2nc(-c3ccc4cc(-c5cccc6ccccc56)ccc4c3)nc(-c3cccc4c3oc3ccccc34)n2)cc1]

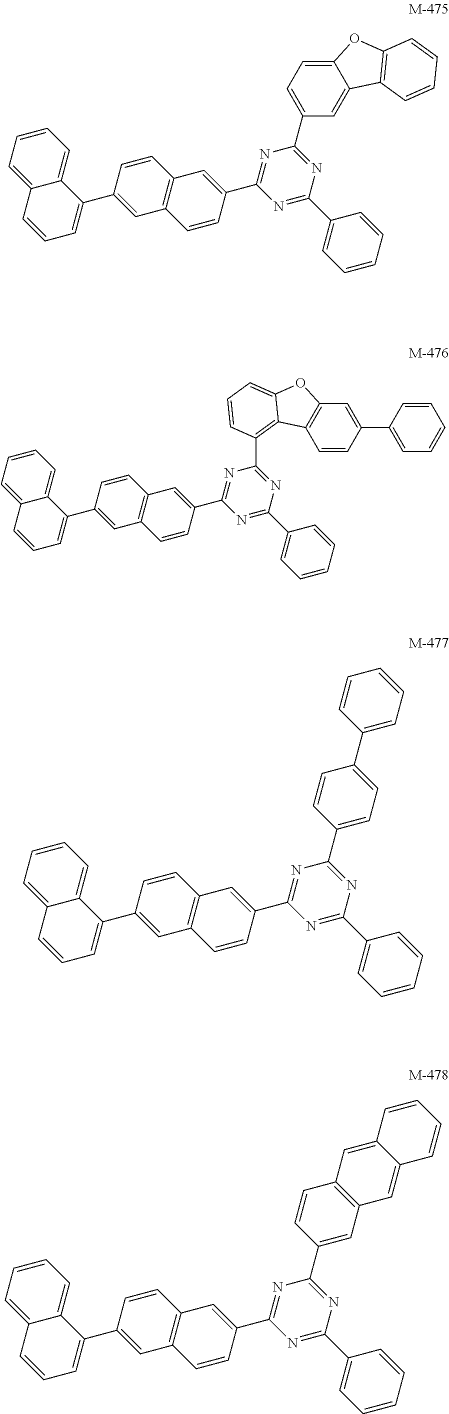 [SMILES: c1ccc(-c2nc(-c3ccc4cc(-c5cccc6ccccc56)ccc4c3)nc(-c3ccc4oc5ccccc5c4c3)n2)cc1 ; c1ccc(-c2ccc3c(c2)oc2cccc(-c4nc(-c5ccccc5)nc(-c5ccc6cc(-c7cccc8ccccc78)ccc6c5)n4)c23)cc1 ; c1ccc(-c2ccc(-c3nc(-c4ccccc4)nc(-c4ccc5cc(-c6cccc7ccccc67)ccc5c4)n3)cc2)cc1 ; c1ccc(-c2nc(-c3ccc4cc(-c5cccc6ccccc56)ccc4c3)nc(-c3ccc4cc5ccccc5cc4c3)n2)cc1]

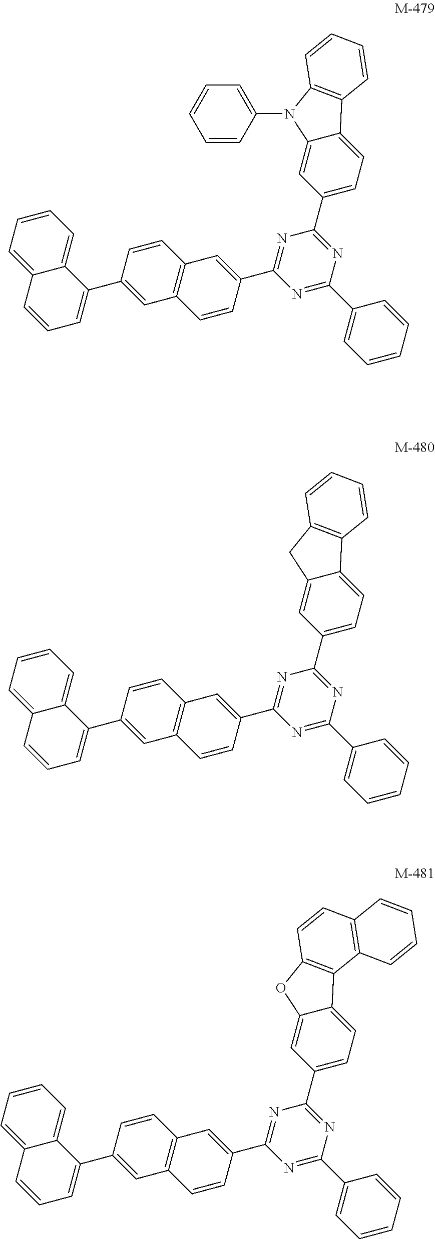 [SMILES: c1ccc(-c2nc(-c3ccc4cc(-c5cccc6ccccc56)ccc4c3)nc(-c3ccc4c5ccccc5n(-c5ccccc5)c4c3)n2)cc1 ; c1ccc(-c2nc(-c3ccc4c(c3)Cc3ccccc3-4)nc(-c3ccc4cc(-c5cccc6ccccc56)ccc4c3)n2)cc1 ; c1ccc(-c2nc(-c3ccc4cc(-c5cccc6ccccc56)ccc4c3)nc(-c3ccc4c(c3)oc3ccc5ccccc5c34)n2)cc1]

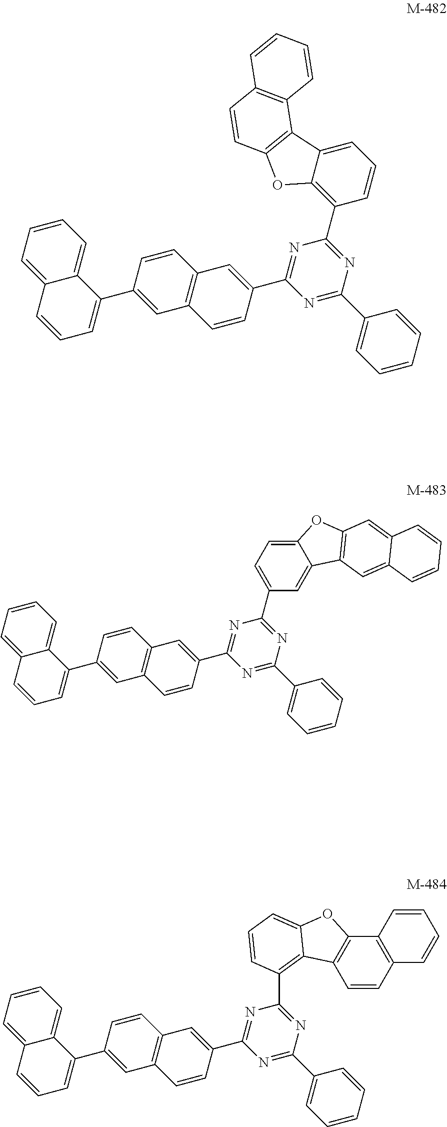 [SMILES: c1ccc(-c2nc(-c3ccc4cc(-c5cccc6ccccc56)ccc4c3)nc(-c3cccc4c3oc3ccc5ccccc5c34)n2)cc1 ; c1ccc(-c2nc(-c3ccc4cc(-c5cccc6ccccc56)ccc4c3)nc(-c3ccc4oc5cc6ccccc6cc5c4c3)n2)cc1 ; c1ccc(-c2nc(-c3ccc4cc(-c5cccc6ccccc56)ccc4c3)nc(-c3cccc4oc5c6ccccc6ccc5c34)n2)cc1]

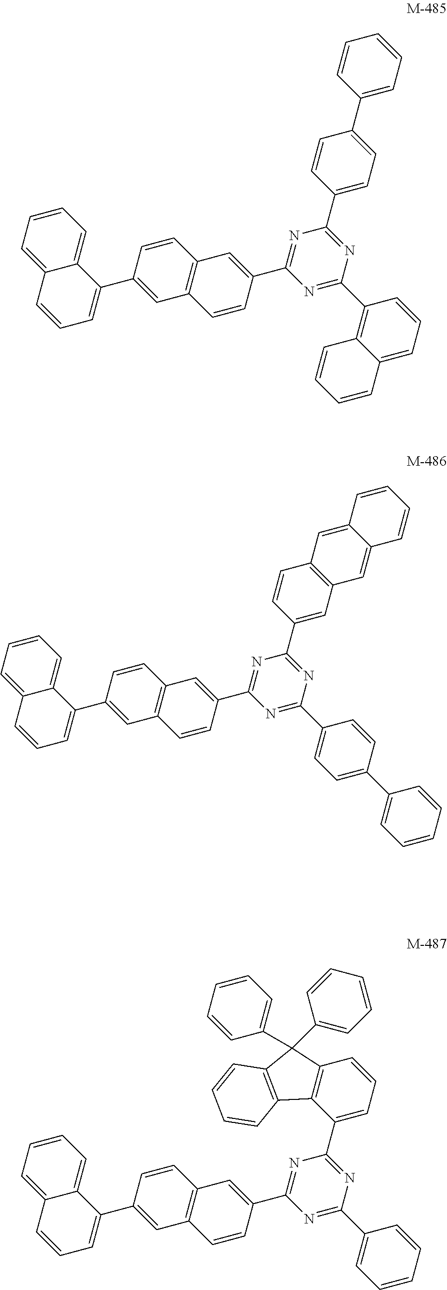 [SMILES: c1ccc(-c2ccc(-c3nc(-c4ccc5cc(-c6cccc7ccccc67)ccc5c4)nc(-c4cccc5ccccc45)n3)cc2)cc1 ; c1ccc(-c2ccc(-c3nc(-c4ccc5cc(-c6cccc7ccccc67)ccc5c4)nc(-c4ccc5cc6ccccc6cc5c4)n3)cc2)cc1 ; c1ccc(-c2nc(-c3ccc4cc(-c5cccc6ccccc56)ccc4c3)nc(-c3cccc4c3-c3ccccc3C4(c3ccccc3)c3ccccc3)n2)cc1]

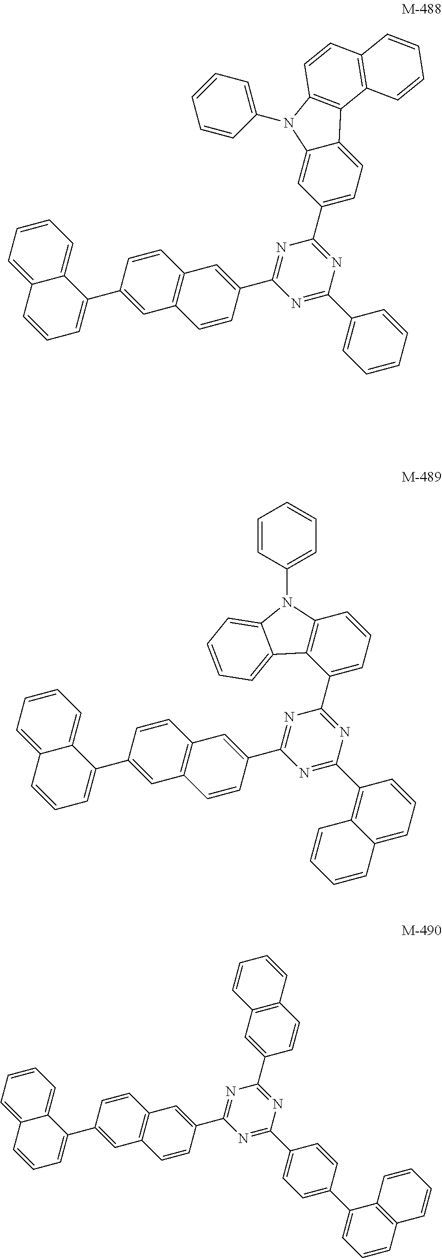 [SMILES: c1ccc(-c2nc(-c3ccc4cc(-c5cccc6ccccc56)ccc4c3)nc(-c3ccc4c5c6ccccc6ccc5n(-c5ccccc5)c4c3)n2)cc1 ; c1ccc(-n2c3ccccc3c3c(-c4nc(-c5ccc6cc(-c7cccc8ccccc78)ccc6c5)nc(-c5cccc6ccccc56)n4)cccc32)cc1 ; c1ccc2cc(-c3nc(-c4ccc(-c5cccc6ccccc56)cc4)nc(-c4ccc5cc(-c6cccc7ccccc67)ccc5c4)n3)ccc2c1]

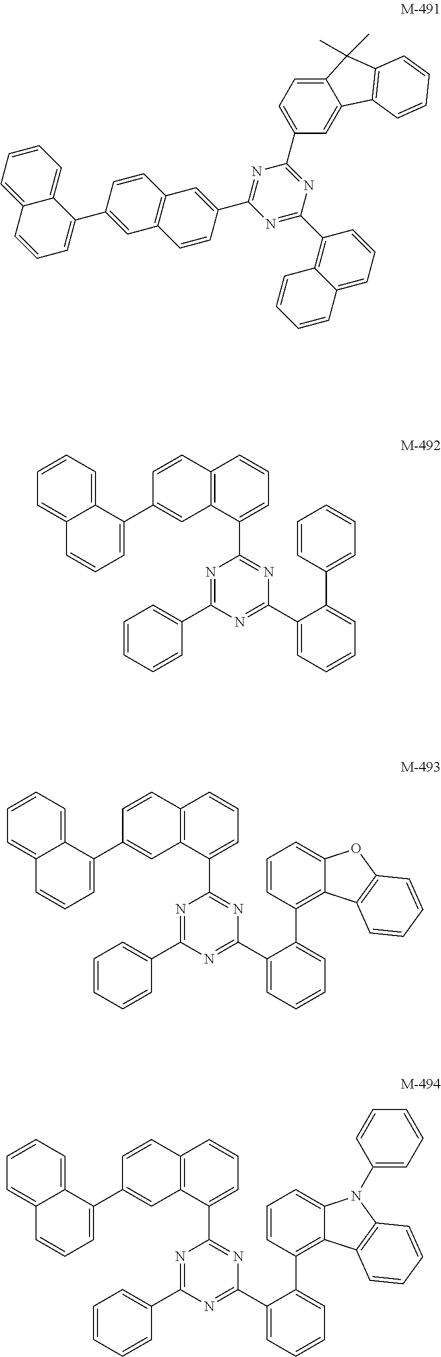 [SMILES: CC1(C)c2ccccc2-c2cc(-c3nc(-c4ccc5cc(-c6cccc7ccccc67)ccc5c4)nc(-c4cccc5ccccc45)n3)ccc21 ; c1ccc(-c2nc(-c3ccccc3-c3ccccc3)nc(-c3cccc4ccc(-c5cccc6ccccc56)cc34)n2)cc1 ; c1ccc(-c2nc(-c3ccccc3-c3cccc4oc5ccccc5c34)nc(-c3cccc4ccc(-c5cccc6ccccc56)cc34)n2)cc1 ; c1ccc(-c2nc(-c3ccccc3-c3cccc4c3c3ccccc3n4-c3ccccc3)nc(-c3cccc4ccc(-c5cccc6ccccc56)cc34)n2)cc1]

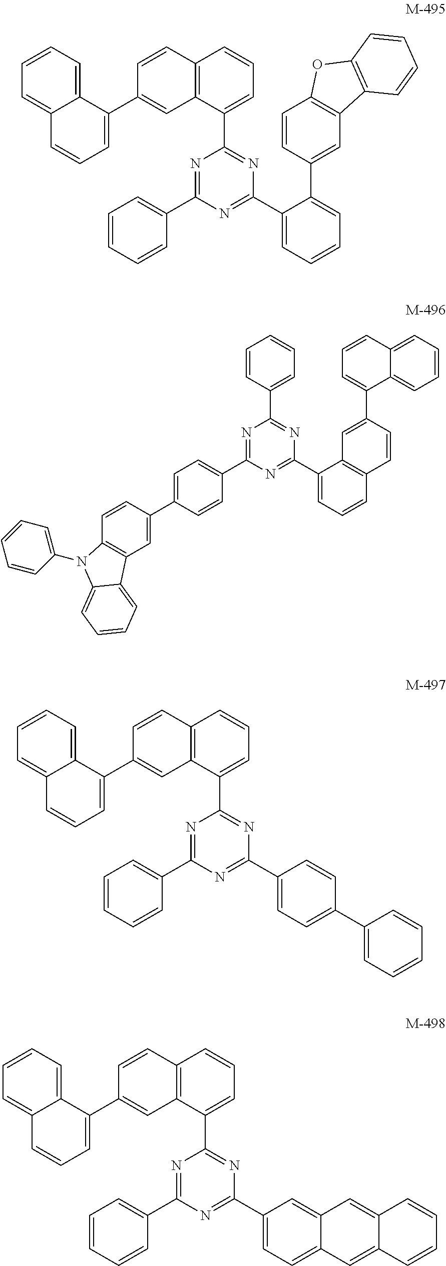 [SMILES: c1ccc(-c2nc(-c3ccccc3-c3ccc4oc5ccccc5c4c3)nc(-c3cccc4ccc(-c5cccc6ccccc56)cc34)n2)cc1 ; c1ccc(-c2nc(-c3ccc(-c4ccc5c(c4)c4ccccc4n5-c4ccccc4)cc3)nc(-c3cccc4ccc(-c5cccc6ccccc56)cc34)n2)cc1 ; c1ccc(-c2ccc(-c3nc(-c4ccccc4)nc(-c4cccc5ccc(-c6cccc7ccccc67)cc45)n3)cc2)cc1 ; c1ccc(-c2nc(-c3ccc4cc5ccccc5cc4c3)nc(-c3cccc4ccc(-c5cccc6ccccc56)cc34)n2)cc1]

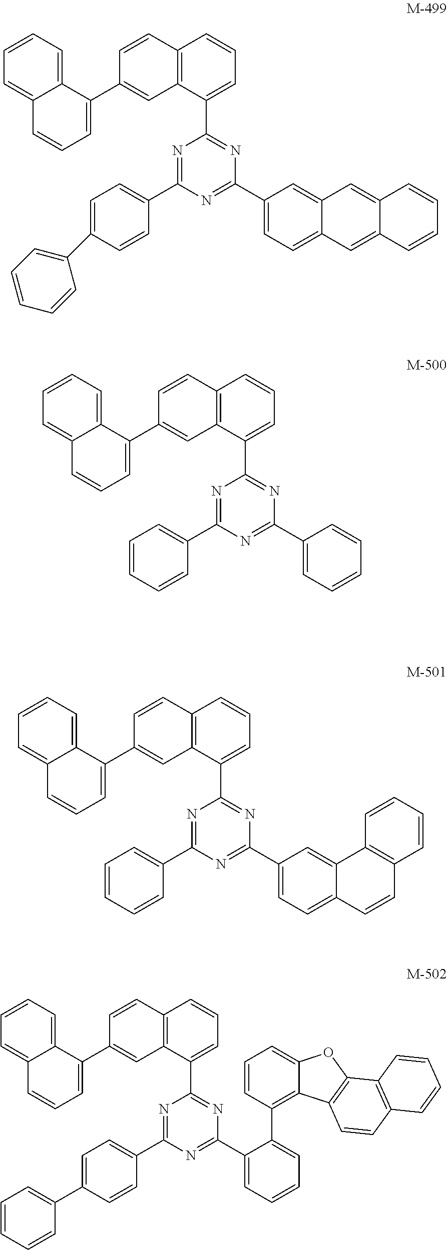 [SMILES: c1ccc(-c2ccc(-c3nc(-c4ccc5cc6ccccc6cc5c4)nc(-c4cccc5ccc(-c6cccc7ccccc67)cc45)n3)cc2)cc1 ; c1ccc(-c2nc(-c3ccccc3)nc(-c3cccc4ccc(-c5cccc6ccccc56)cc34)n2)cc1 ; c1ccc(-c2nc(-c3ccc4ccc5ccccc5c4c3)nc(-c3cccc4ccc(-c5cccc6ccccc56)cc34)n2)cc1 ; c1ccc(-c2ccc(-c3nc(-c4ccccc4-c4cccc5oc6c7ccccc7ccc6c45)nc(-c4cccc5ccc(-c6cccc7ccccc67)cc45)n3)cc2)cc1]

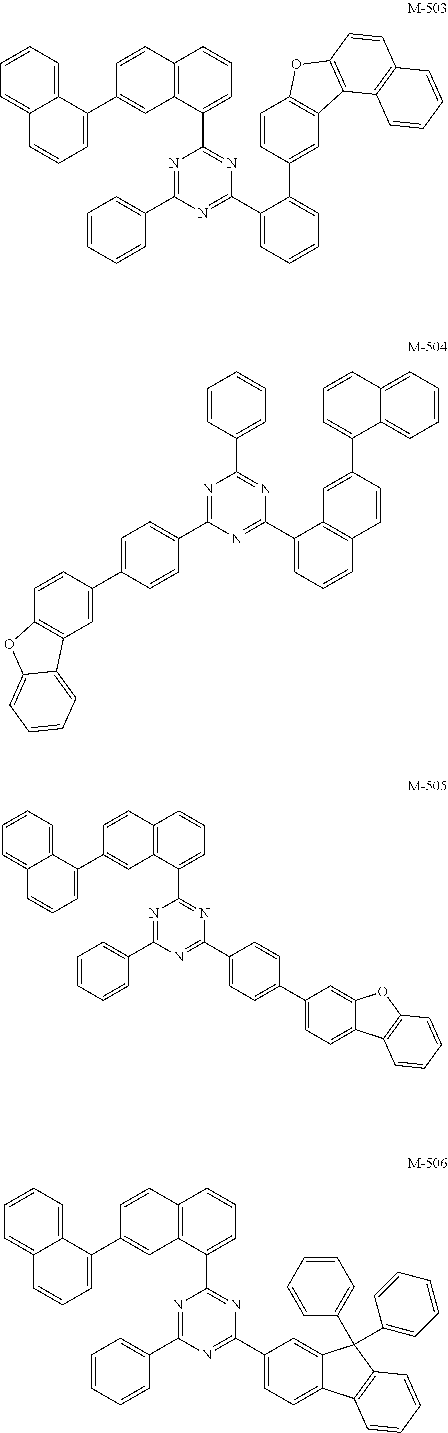 [SMILES: c1ccc(-c2nc(-c3ccccc3-c3ccc4oc5ccc6ccccc6c5c4c3)nc(-c3cccc4ccc(-c5cccc6ccccc56)cc34)n2)cc1 ; c1ccc(-c2nc(-c3ccc(-c4ccc5oc6ccccc6c5c4)cc3)nc(-c3cccc4ccc(-c5cccc6ccccc56)cc34)n2)cc1 ; c1ccc(-c2nc(-c3ccc(-c4ccc5c(c4)oc4ccccc45)cc3)nc(-c3cccc4ccc(-c5cccc6ccccc56)cc34)n2)cc1 ; c1ccc(-c2nc(-c3ccc4c(c3)C(c3ccccc3)(c3ccccc3)c3ccccc3-4)nc(-c3cccc4ccc(-c5cccc6ccccc56)cc34)n2)cc1]

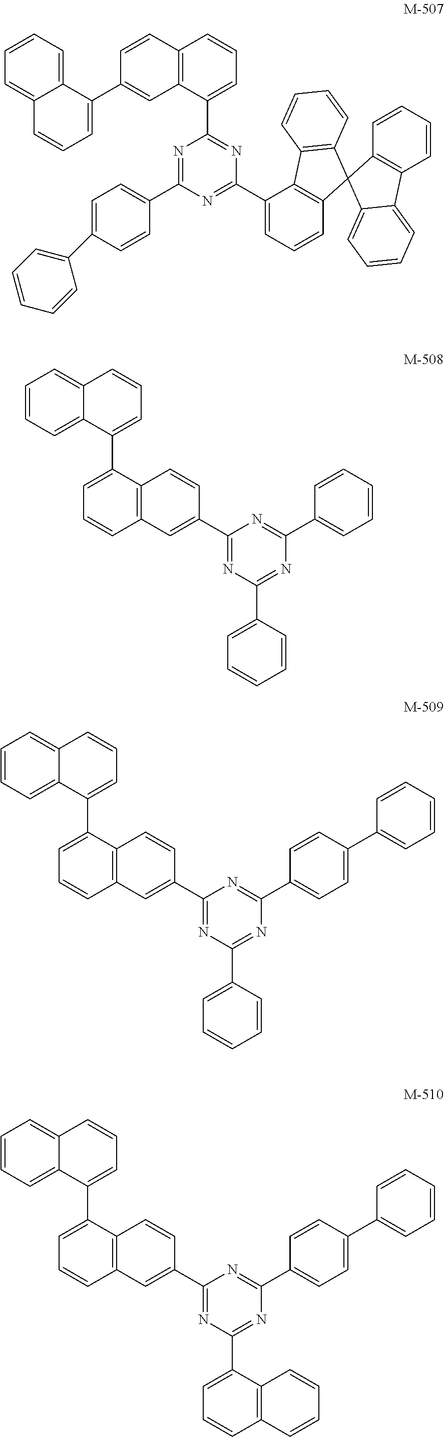 [SMILES: c1ccc(-c2ccc(-c3nc(-c4cccc5c4-c4ccccc4C54c5ccccc5-c5ccccc54)nc(-c4cccc5ccc(-c6cccc7ccccc67)cc45)n3)cc2)cc1 ; c1ccc(-c2nc(-c3ccccc3)nc(-c3ccc4c(-c5cccc6ccccc56)cccc4c3)n2)cc1 ; c1ccc(-c2ccc(-c3nc(-c4ccccc4)nc(-c4ccc5c(-c6cccc7ccccc67)cccc5c4)n3)cc2)cc1 ; c1ccc(-c2ccc(-c3nc(-c4ccc5c(-c6cccc7ccccc67)cccc5c4)nc(-c4cccc5ccccc45)n3)cc2)cc1]

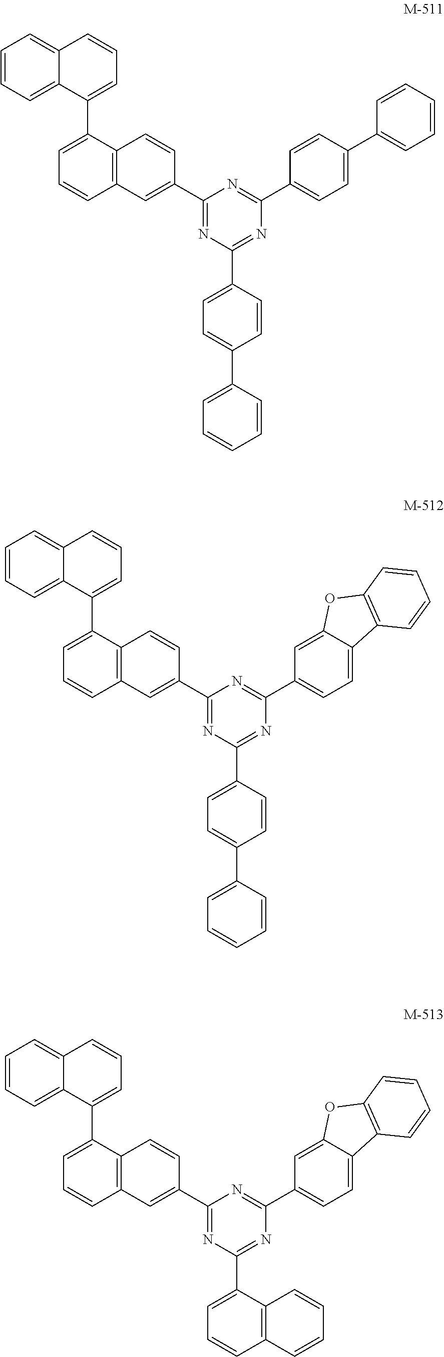 [SMILES: c1ccc(-c2ccc(-c3nc(-c4ccc(-c5ccccc5)cc4)nc(-c4ccc5c(-c6cccc7ccccc67)cccc5c4)n3)cc2)cc1 ; c1ccc(-c2ccc(-c3nc(-c4ccc5c(-c6cccc7ccccc67)cccc5c4)nc(-c4ccc5c(c4)oc4ccccc45)n3)cc2)cc1 ; c1ccc2c(-c3nc(-c4ccc5c(-c6cccc7ccccc67)cccc5c4)nc(-c4ccc5c(c4)oc4ccccc45)n3)cccc2c1]

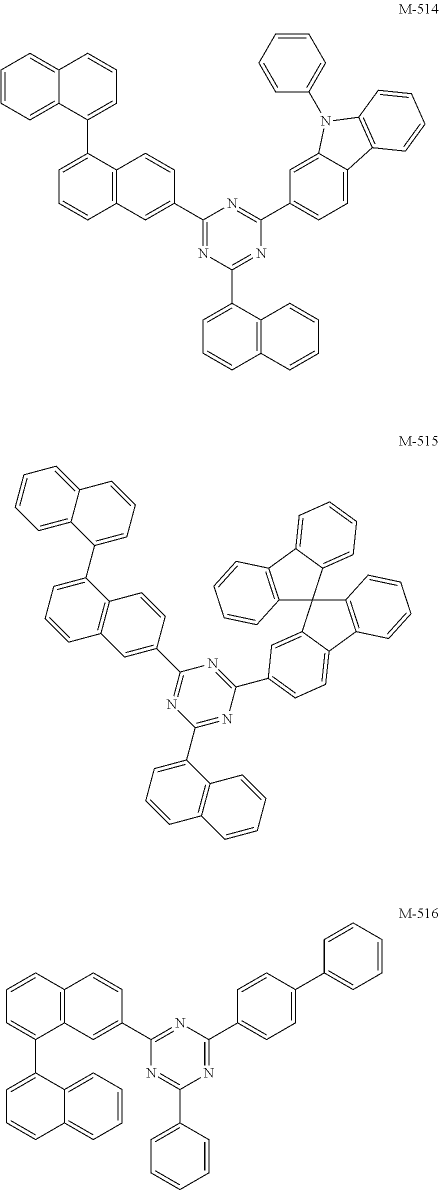 [SMILES: c1ccc(-n2c3ccccc3c3ccc(-c4nc(-c5ccc6c(-c7cccc8ccccc78)cccc6c5)nc(-c5cccc6ccccc56)n4)cc32)cc1 ; c1ccc2c(c1)-c1ccccc1C21c2ccccc2-c2ccc(-c3nc(-c4ccc5c(-c6cccc7ccccc67)cccc5c4)nc(-c4cccc5ccccc45)n3)cc21 ; c1ccc(-c2ccc(-c3nc(-c4ccccc4)nc(-c4ccc5cccc(-c6cccc7ccccc67)c5c4)n3)cc2)cc1]

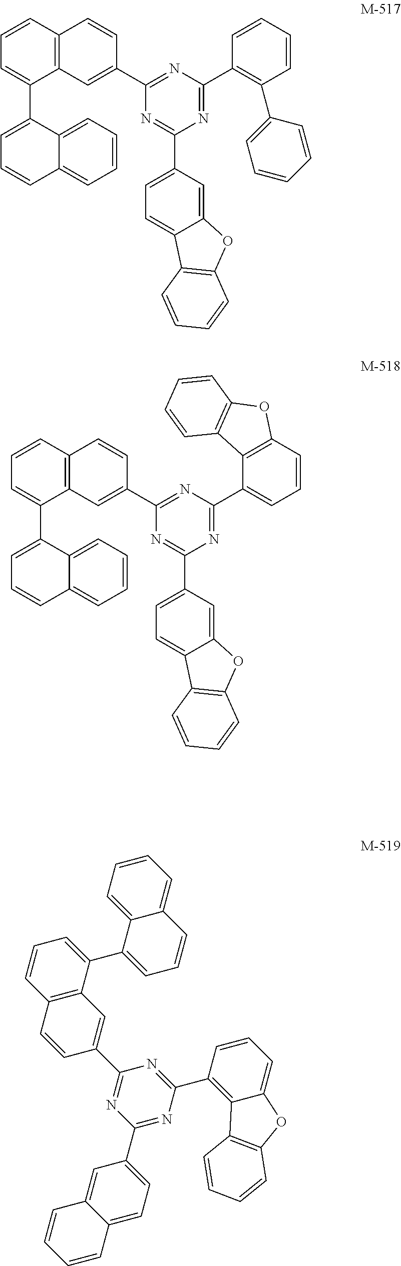 [SMILES: c1ccc(-c2ccccc2-c2nc(-c3ccc4c(c3)oc3ccccc34)nc(-c3ccc4cccc(-c5cccc6ccccc56)c4c3)n2)cc1 ; c1ccc2c(-c3cccc4ccc(-c5nc(-c6ccc7c(c6)oc6ccccc67)nc(-c6cccc7oc8ccccc8c67)n5)cc34)cccc2c1 ; c1ccc2cc(-c3nc(-c4ccc5cccc(-c6cccc7ccccc67)c5c4)nc(-c4cccc5oc6ccccc6c45)n3)ccc2c1]

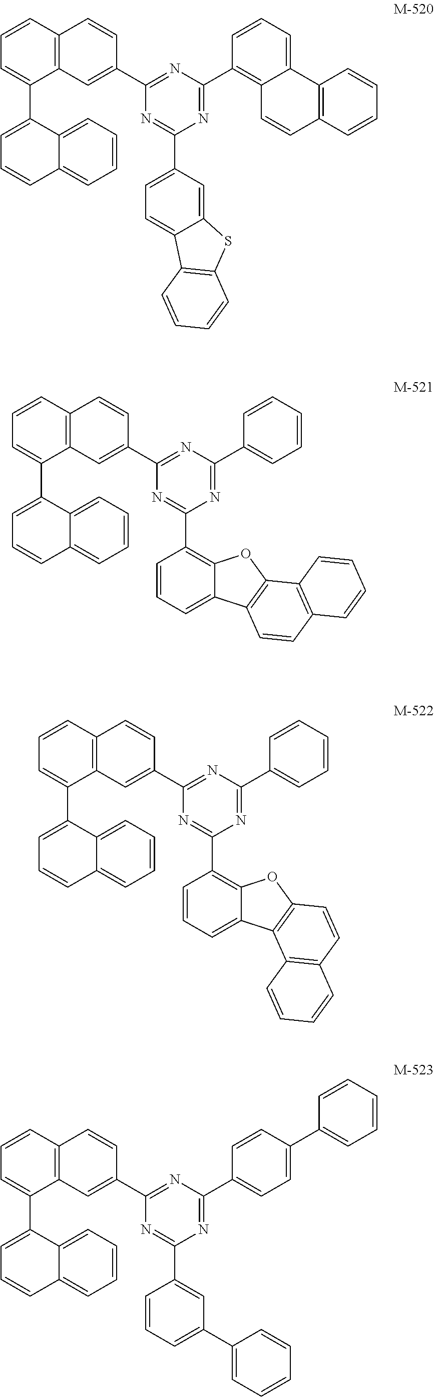 [SMILES: c1ccc2c(-c3cccc4ccc(-c5nc(-c6ccc7c(c6)sc6ccccc67)nc(-c6cccc7c6ccc6ccccc67)n5)cc34)cccc2c1 ; c1ccc(-c2nc(-c3ccc4cccc(-c5cccc6ccccc56)c4c3)nc(-c3cccc4c3oc3c5ccccc5ccc43)n2)cc1 ; c1ccc(-c2nc(-c3ccc4cccc(-c5cccc6ccccc56)c4c3)nc(-c3cccc4c3oc3ccc5ccccc5c34)n2)cc1 ; c1ccc(-c2ccc(-c3nc(-c4cccc(-c5ccccc5)c4)nc(-c4ccc5cccc(-c6cccc7ccccc67)c5c4)n3)cc2)cc1]

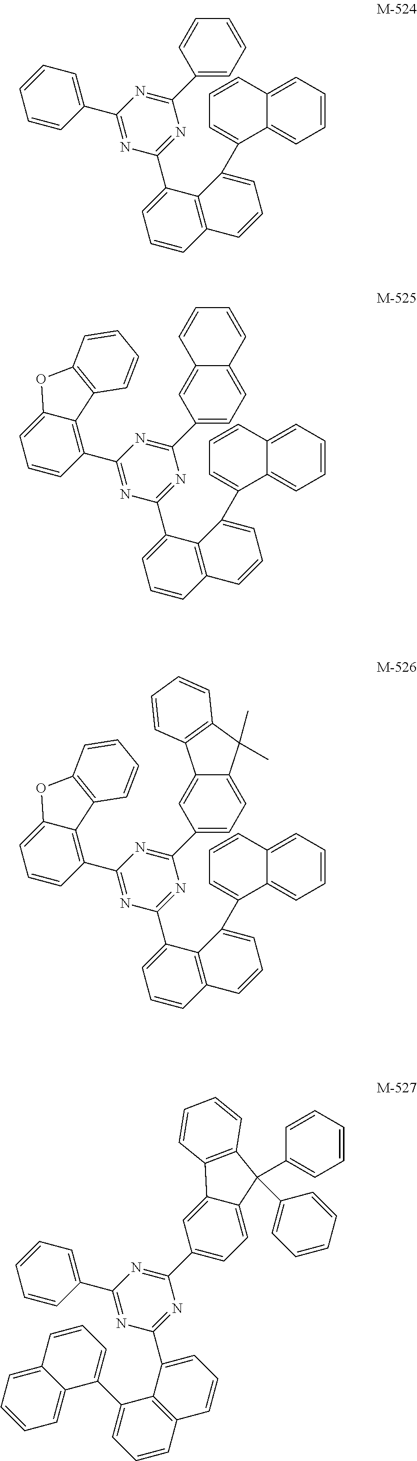 [SMILES: c1ccc(-c2nc(-c3ccccc3)nc(-c3cccc4cccc(-c5cccc6ccccc56)c34)n2)cc1 ; c1ccc2cc(-c3nc(-c4cccc5cccc(-c6cccc7ccccc67)c45)nc(-c4cccc5oc6ccccc6c45)n3)ccc2c1 ; CC1(C)c2ccccc2-c2cc(-c3nc(-c4cccc5cccc(-c6cccc7ccccc67)c45)nc(-c4cccc5oc6ccccc6c45)n3)ccc21 ; c1ccc(-c2nc(-c3ccc4c(c3)-c3ccccc3C4(c3ccccc3)c3ccccc3)nc(-c3cccc4cccc(-c5cccc6ccccc56)c34)n2)cc1]

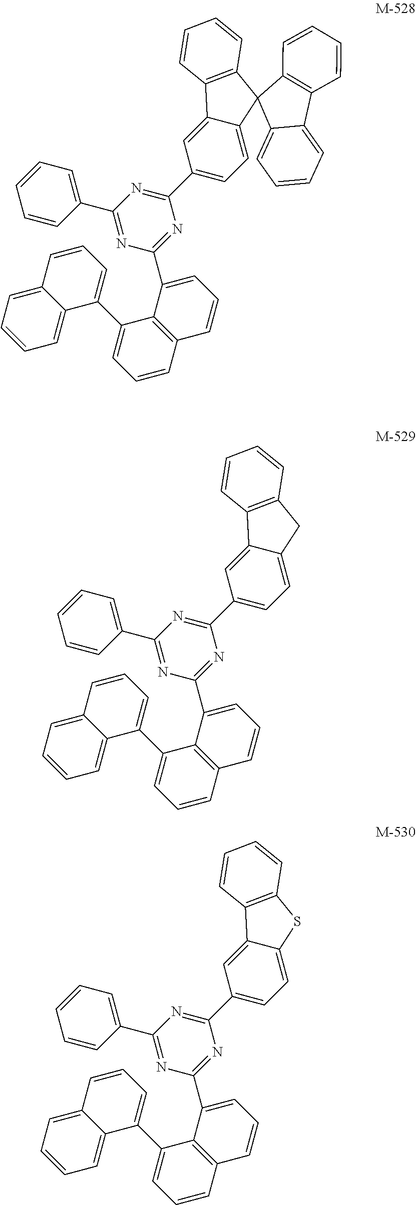 [SMILES: c1ccc(-c2nc(-c3ccc4c(c3)-c3ccccc3C43c4ccccc4-c4ccccc43)nc(-c3cccc4cccc(-c5cccc6ccccc56)c34)n2)cc1 ; c1ccc(-c2nc(-c3ccc4c(c3)-c3ccccc3C4)nc(-c3cccc4cccc(-c5cccc6ccccc56)c34)n2)cc1 ; c1ccc(-c2nc(-c3ccc4sc5ccccc5c4c3)nc(-c3cccc4cccc(-c5cccc6ccccc56)c34)n2)cc1]

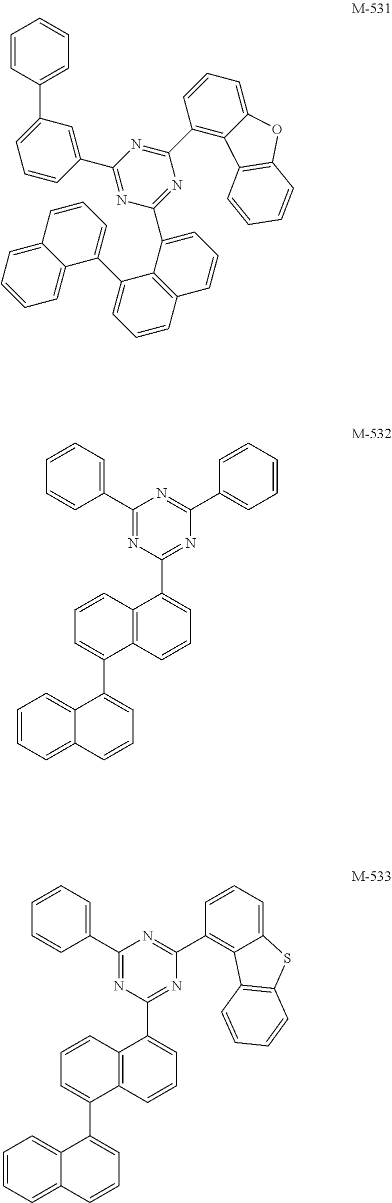 [SMILES: c1ccc(-c2cccc(-c3nc(-c4cccc5cccc(-c6cccc7ccccc67)c45)nc(-c4cccc5oc6ccccc6c45)n3)c2)cc1 ; c1ccc(-c2nc(-c3ccccc3)nc(-c3cccc4c(-c5cccc6ccccc56)cccc34)n2)cc1 ; c1ccc(-c2nc(-c3cccc4c(-c5cccc6ccccc56)cccc34)nc(-c3cccc4sc5ccccc5c34)n2)cc1]

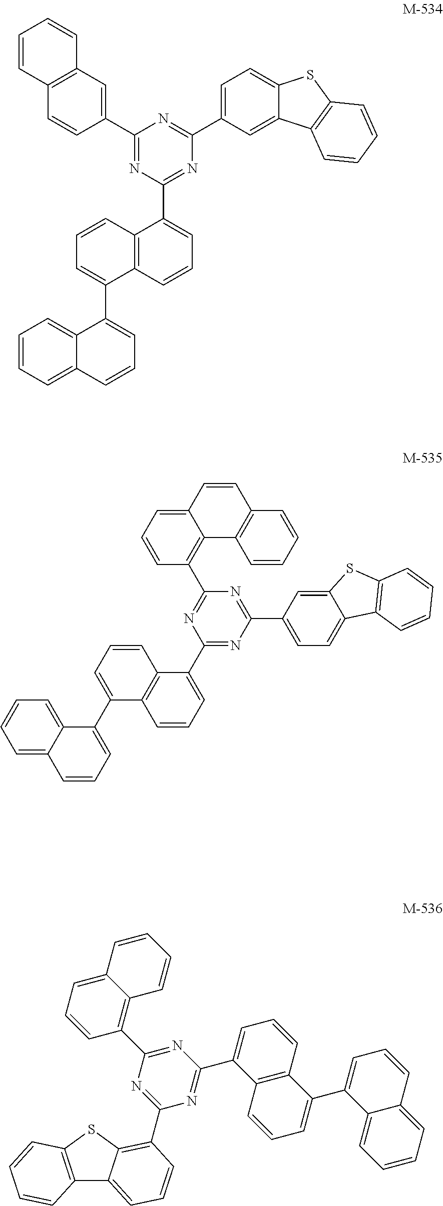 [SMILES: c1ccc2cc(-c3nc(-c4ccc5sc6ccccc6c5c4)nc(-c4cccc5c(-c6cccc7ccccc67)cccc45)n3)ccc2c1 ; c1ccc2c(-c3cccc4c(-c5nc(-c6ccc7c(c6)sc6ccccc67)nc(-c6cccc7ccc8ccccc8c67)n5)cccc34)cccc2c1 ; c1ccc2c(-c3nc(-c4cccc5c(-c6cccc7ccccc67)cccc45)nc(-c4cccc5c4sc4ccccc45)n3)cccc2c1]

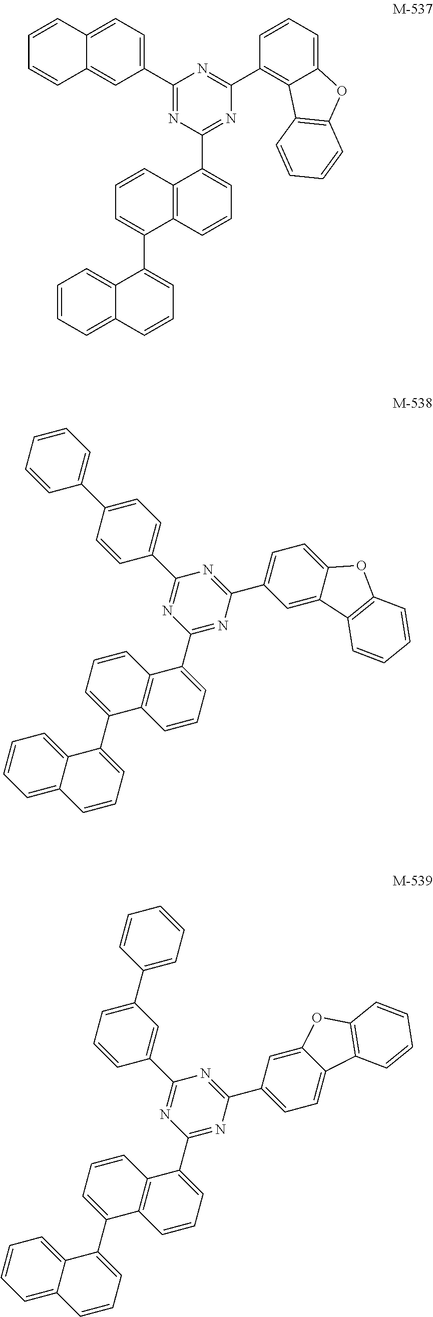 [SMILES: c1ccc2cc(-c3nc(-c4cccc5c(-c6cccc7ccccc67)cccc45)nc(-c4cccc5oc6ccccc6c45)n3)ccc2c1 ; c1ccc(-c2ccc(-c3nc(-c4ccc5oc6ccccc6c5c4)nc(-c4cccc5c(-c6cccc7ccccc67)cccc45)n3)cc2)cc1 ; c1ccc(-c2cccc(-c3nc(-c4ccc5c(c4)oc4ccccc45)nc(-c4cccc5c(-c6cccc7ccccc67)cccc45)n3)c2)cc1]

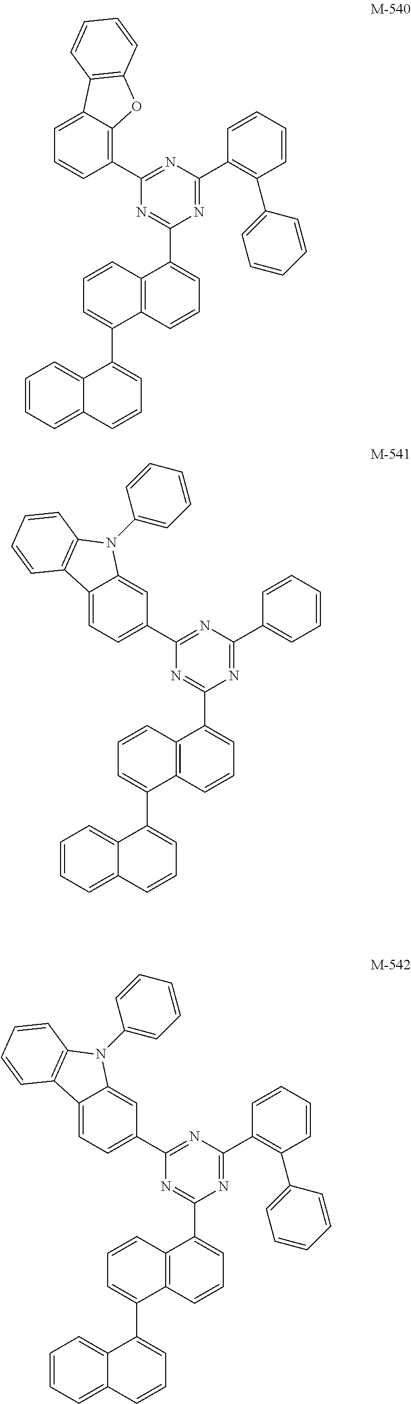 [SMILES: c1ccc(-c2ccccc2-c2nc(-c3cccc4c(-c5cccc6ccccc56)cccc34)nc(-c3cccc4c3oc3ccccc34)n2)cc1 ; c1ccc(-c2nc(-c3ccc4c5ccccc5n(-c5ccccc5)c4c3)nc(-c3cccc4c(-c5cccc6ccccc56)cccc34)n2)cc1 ; c1ccc(-c2ccccc2-c2nc(-c3ccc4c5ccccc5n(-c5ccccc5)c4c3)nc(-c3cccc4c(-c5cccc6ccccc56)cccc34)n2)cc1]

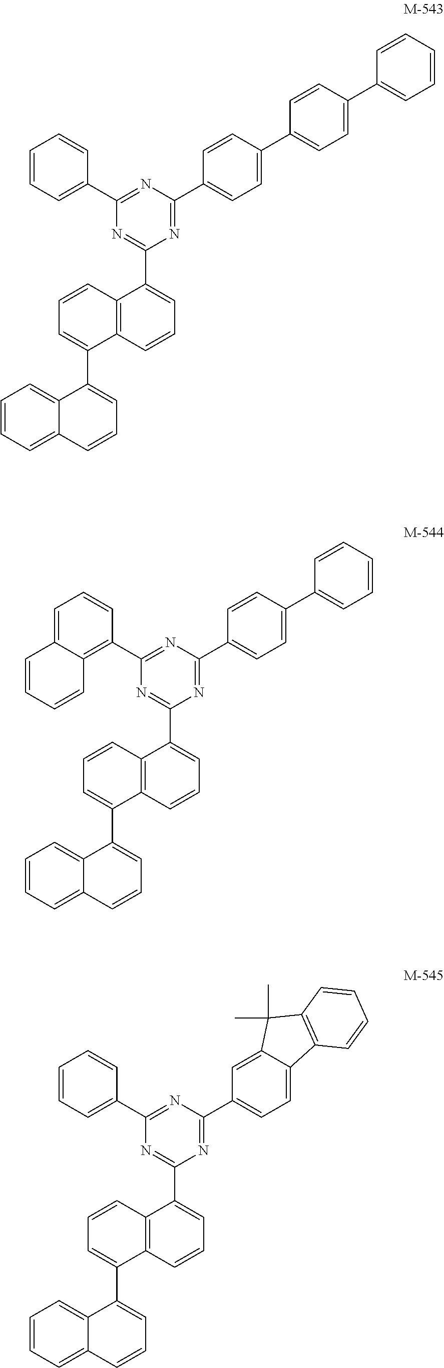 [SMILES: c1ccc(-c2ccc(-c3ccc(-c4nc(-c5ccccc5)nc(-c5cccc6c(-c7cccc8ccccc78)cccc56)n4)cc3)cc2)cc1 ; c1ccc(-c2ccc(-c3nc(-c4cccc5ccccc45)nc(-c4cccc5c(-c6cccc7ccccc67)cccc45)n3)cc2)cc1 ; CC1(C)c2ccccc2-c2ccc(-c3nc(-c4ccccc4)nc(-c4cccc5c(-c6cccc7ccccc67)cccc45)n3)cc21]

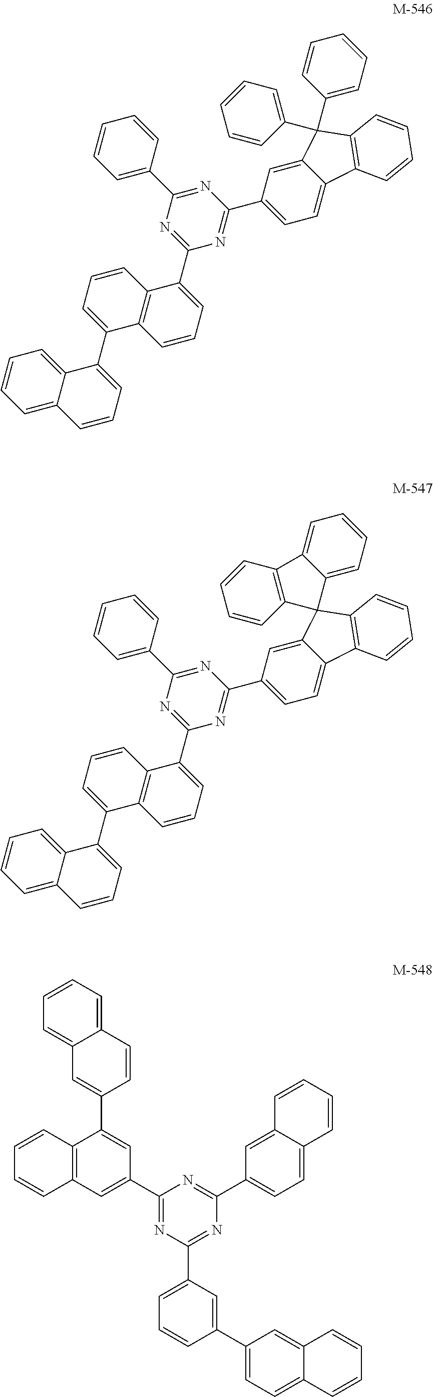 [SMILES: c1ccc(-c2nc(-c3ccc4c(c3)C(c3ccccc3)(c3ccccc3)c3ccccc3-4)nc(-c3cccc4c(-c5cccc6ccccc56)cccc34)n2)cc1 ; c1ccc(-c2nc(-c3ccc4c(c3)C3(c5ccccc5-c5ccccc53)c3ccccc3-4)nc(-c3cccc4c(-c5cccc6ccccc56)cccc34)n2)cc1 ; c1cc(-c2ccc3ccccc3c2)cc(-c2nc(-c3ccc4ccccc4c3)nc(-c3cc(-c4ccc5ccccc5c4)c4ccccc4c3)n2)c1]

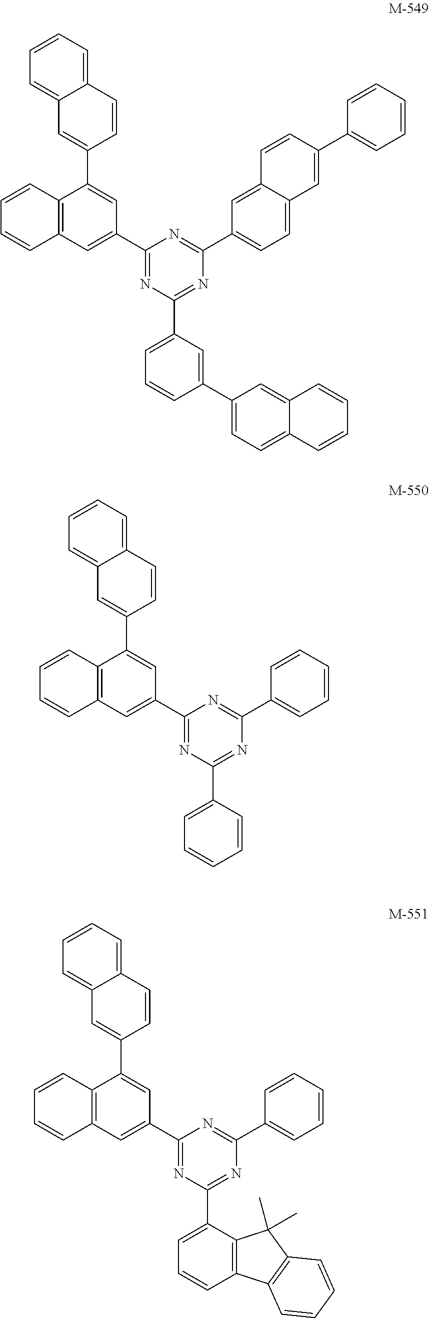 [SMILES: c1ccc(-c2ccc3cc(-c4nc(-c5cccc(-c6ccc7ccccc7c6)c5)nc(-c5cc(-c6ccc7ccccc7c6)c6ccccc6c5)n4)ccc3c2)cc1 ; c1ccc(-c2nc(-c3ccccc3)nc(-c3cc(-c4ccc5ccccc5c4)c4ccccc4c3)n2)cc1 ; CC1(C)c2ccccc2-c2cccc(-c3nc(-c4ccccc4)nc(-c4cc(-c5ccc6ccccc6c5)c5ccccc5c4)n3)c21]

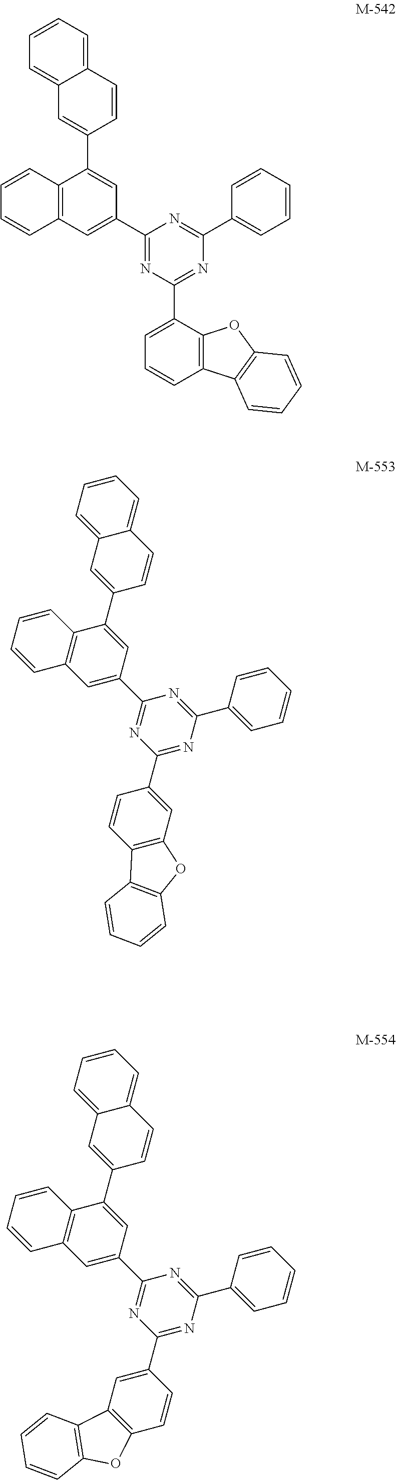 [SMILES: c1ccc(-c2nc(-c3cc(-c4ccc5ccccc5c4)c4ccccc4c3)nc(-c3cccc4c3oc3ccccc34)n2)cc1 ; c1ccc(-c2nc(-c3cc(-c4ccc5ccccc5c4)c4ccccc4c3)nc(-c3ccc4c(c3)oc3ccccc34)n2)cc1 ; c1ccc(-c2nc(-c3cc(-c4ccc5ccccc5c4)c4ccccc4c3)nc(-c3ccc4oc5ccccc5c4c3)n2)cc1]

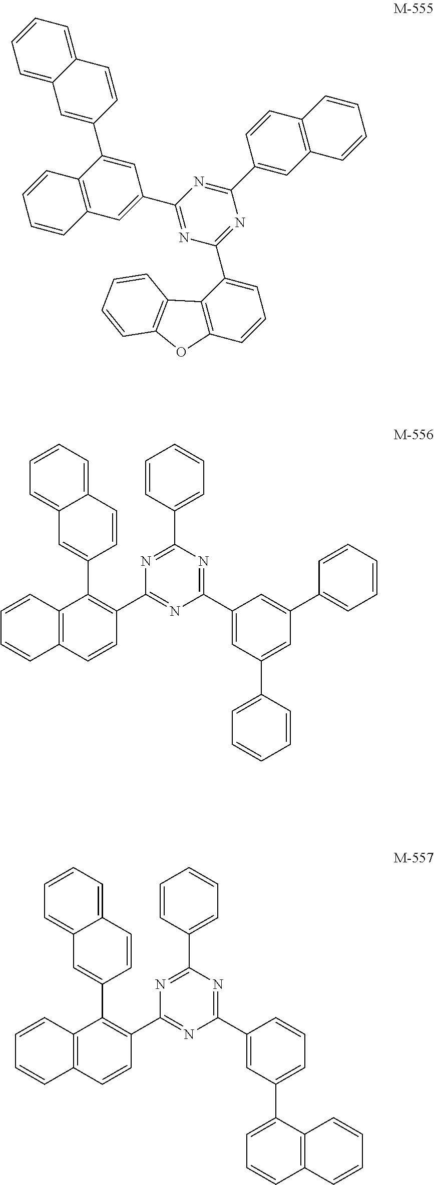 [SMILES: c1ccc2cc(-c3nc(-c4cc(-c5ccc6ccccc6c5)c5ccccc5c4)nc(-c4cccc5oc6ccccc6c45)n3)ccc2c1 ; c1ccc(-c2cc(-c3ccccc3)cc(-c3nc(-c4ccccc4)nc(-c4ccc5ccccc5c4-c4ccc5ccccc5c4)n3)c2)cc1 ; c1ccc(-c2nc(-c3cccc(-c4cccc5ccccc45)c3)nc(-c3ccc4ccccc4c3-c3ccc4ccccc4c3)n2)cc1]

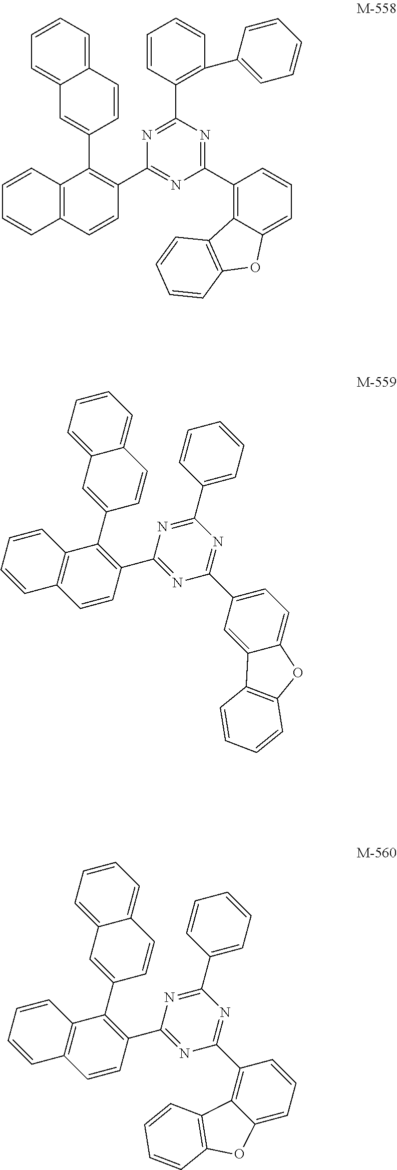 [SMILES: c1ccc(-c2ccccc2-c2nc(-c3ccc4ccccc4c3-c3ccc4ccccc4c3)nc(-c3cccc4oc5ccccc5c34)n2)cc1 ; c1ccc(-c2nc(-c3ccc4oc5ccccc5c4c3)nc(-c3ccc4ccccc4c3-c3ccc4ccccc4c3)n2)cc1 ; c1ccc(-c2nc(-c3ccc4ccccc4c3-c3ccc4ccccc4c3)nc(-c3cccc4oc5ccccc5c34)n2)cc1]

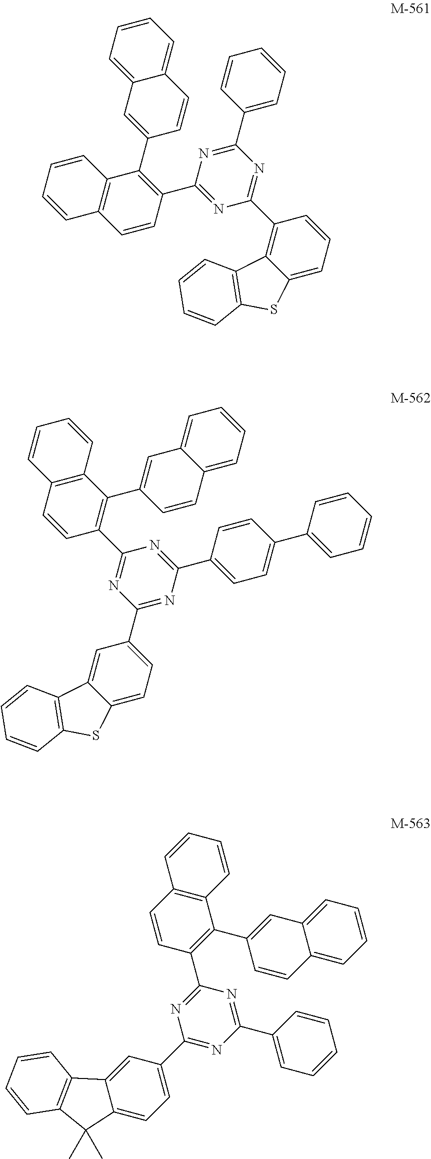 [SMILES: c1ccc(-c2nc(-c3ccc4ccccc4c3-c3ccc4ccccc4c3)nc(-c3cccc4sc5ccccc5c34)n2)cc1 ; c1ccc(-c2ccc(-c3nc(-c4ccc5sc6ccccc6c5c4)nc(-c4ccc5ccccc5c4-c4ccc5ccccc5c4)n3)cc2)cc1 ; CC1(C)c2ccccc2-c2cc(-c3nc(-c4ccccc4)nc(-c4ccc5ccccc5c4-c4ccc5ccccc5c4)n3)ccc21]

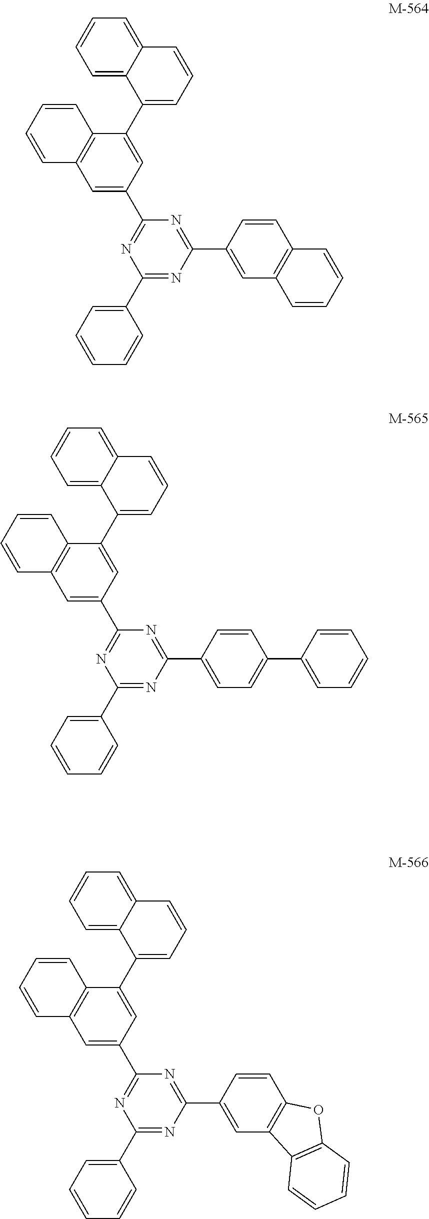 [SMILES: c1ccc(-c2nc(-c3ccc4ccccc4c3)nc(-c3cc(-c4cccc5ccccc45)c4ccccc4c3)n2)cc1 ; c1ccc(-c2ccc(-c3nc(-c4ccccc4)nc(-c4cc(-c5cccc6ccccc56)c5ccccc5c4)n3)cc2)cc1 ; c1ccc(-c2nc(-c3cc(-c4cccc5ccccc45)c4ccccc4c3)nc(-c3ccc4oc5ccccc5c4c3)n2)cc1]

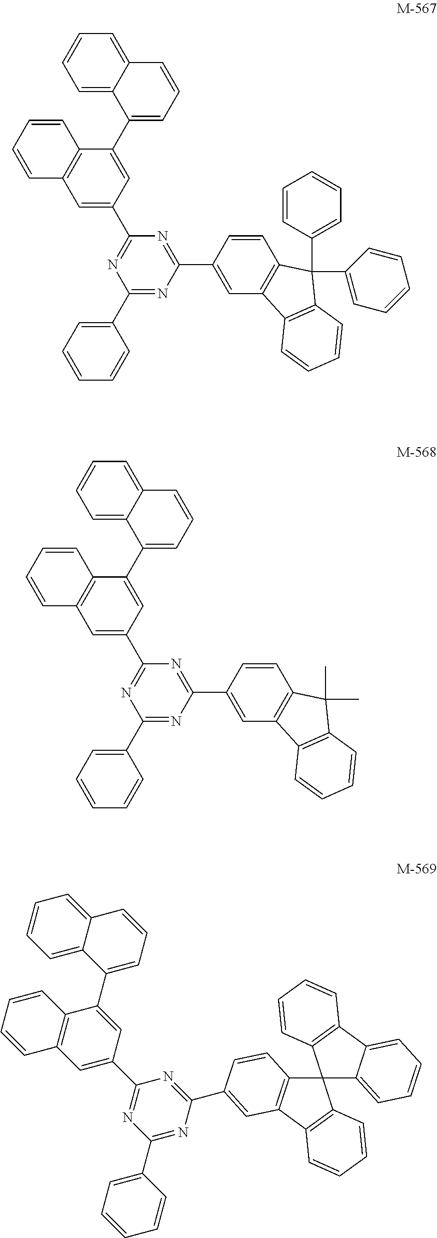 [SMILES: c1ccc(-c2nc(-c3ccc4c(c3)-c3ccccc3C4(c3ccccc3)c3ccccc3)nc(-c3cc(-c4cccc5ccccc45)c4ccccc4c3)n2)cc1 ; CC1(C)c2ccccc2-c2cc(-c3nc(-c4ccccc4)nc(-c4cc(-c5cccc6ccccc56)c5ccccc5c4)n3)ccc21 ; c1ccc(-c2nc(-c3ccc4c(c3)-c3ccccc3C43c4ccccc4-c4ccccc43)nc(-c3cc(-c4cccc5ccccc45)c4ccccc4c3)n2)cc1]

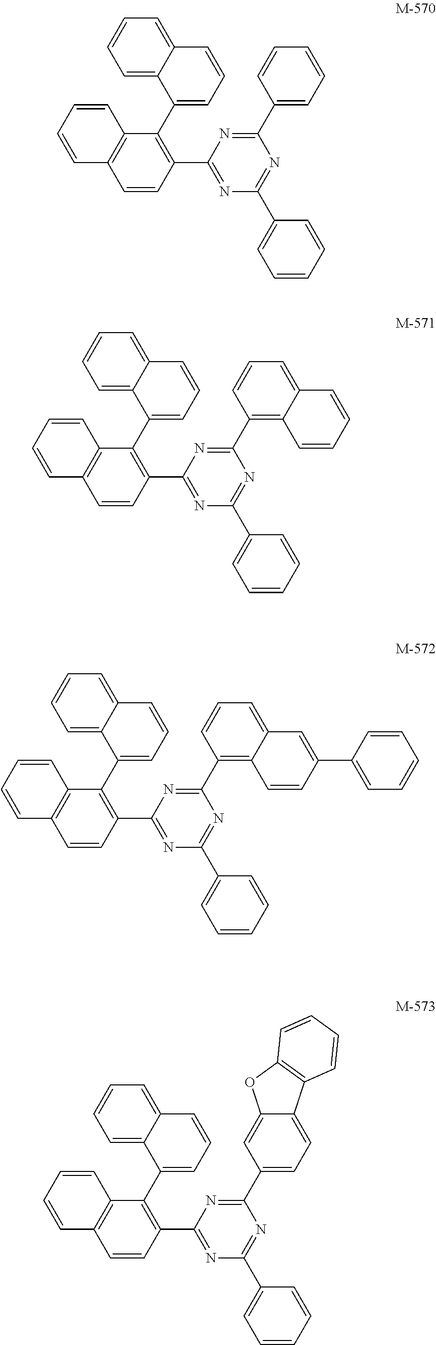 [SMILES: c1ccc(-c2nc(-c3ccccc3)nc(-c3ccc4ccccc4c3-c3cccc4ccccc34)n2)cc1 ; c1ccc(-c2nc(-c3ccc4ccccc4c3-c3cccc4ccccc34)nc(-c3cccc4ccccc34)n2)cc1 ; c1ccc(-c2ccc3c(-c4nc(-c5ccccc5)nc(-c5ccc6ccccc6c5-c5cccc6ccccc56)n4)cccc3c2)cc1 ; c1ccc(-c2nc(-c3ccc4c(c3)oc3ccccc34)nc(-c3ccc4ccccc4c3-c3cccc4ccccc34)n2)cc1]

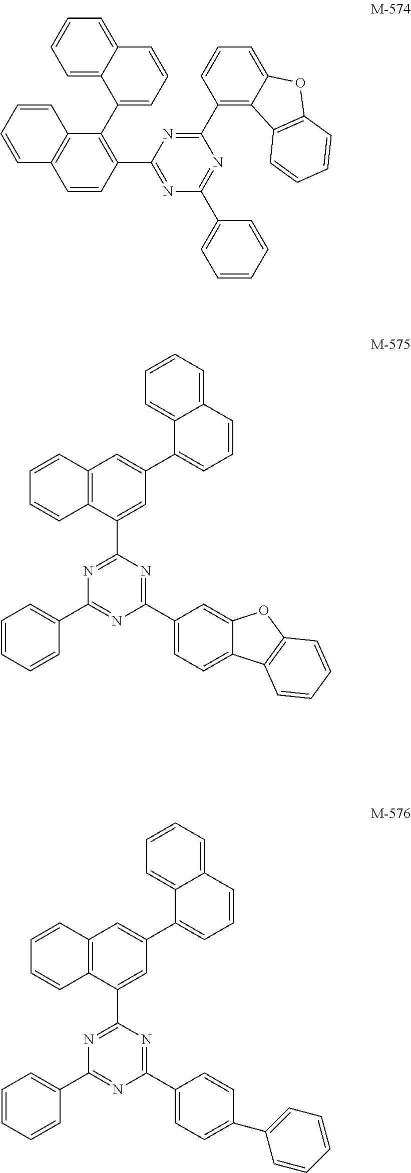 [SMILES: c1ccc(-c2nc(-c3ccc4ccccc4c3-c3cccc4ccccc34)nc(-c3cccc4oc5ccccc5c34)n2)cc1 ; c1ccc(-c2nc(-c3ccc4c(c3)oc3ccccc34)nc(-c3cc(-c4cccc5ccccc45)cc4ccccc34)n2)cc1 ; c1ccc(-c2ccc(-c3nc(-c4ccccc4)nc(-c4cc(-c5cccc6ccccc56)cc5ccccc45)n3)cc2)cc1]

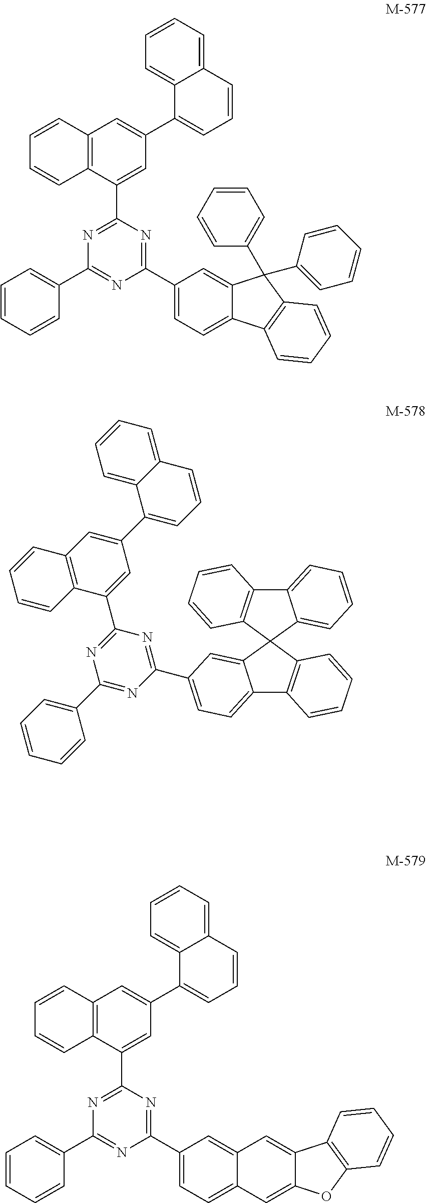 [SMILES: c1ccc(-c2nc(-c3ccc4c(c3)C(c3ccccc3)(c3ccccc3)c3ccccc3-4)nc(-c3cc(-c4cccc5ccccc45)cc4ccccc34)n2)cc1 ; c1ccc(-c2nc(-c3ccc4c(c3)C3(c5ccccc5-c5ccccc53)c3ccccc3-4)nc(-c3cc(-c4cccc5ccccc45)cc4ccccc34)n2)cc1 ; c1ccc(-c2nc(-c3ccc4cc5oc6ccccc6c5cc4c3)nc(-c3cc(-c4cccc5ccccc45)cc4ccccc34)n2)cc1]

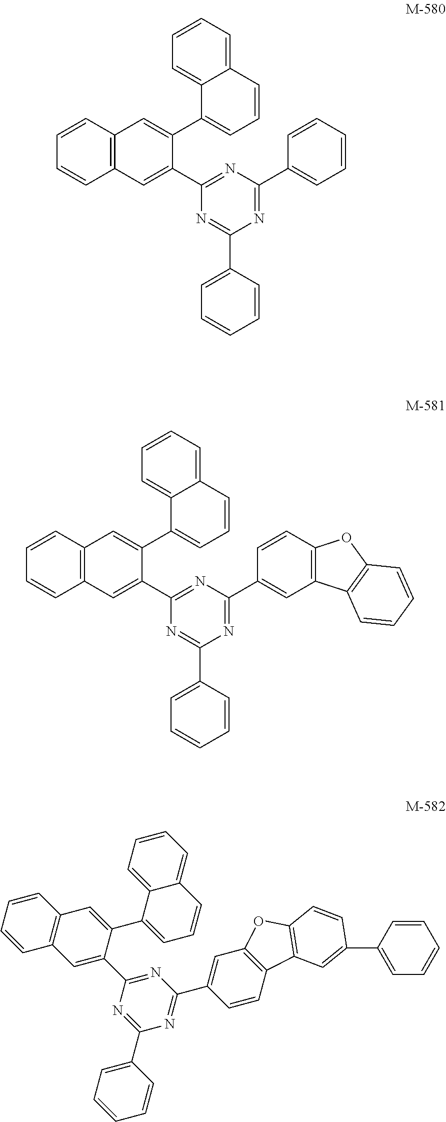 [SMILES: c1ccc(-c2nc(-c3ccccc3)nc(-c3cc4ccccc4cc3-c3cccc4ccccc34)n2)cc1 ; c1ccc(-c2nc(-c3ccc4oc5ccccc5c4c3)nc(-c3cc4ccccc4cc3-c3cccc4ccccc34)n2)cc1 ; c1ccc(-c2ccc3oc4cc(-c5nc(-c6ccccc6)nc(-c6cc7ccccc7cc6-c6cccc7ccccc67)n5)ccc4c3c2)cc1]

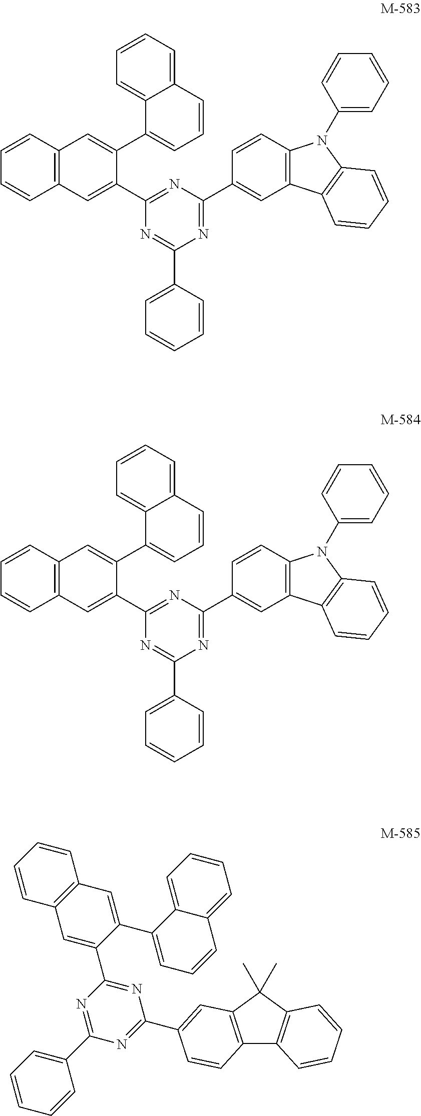 [SMILES: c1ccc(-c2nc(-c3ccc4c(c3)c3ccccc3n4-c3ccccc3)nc(-c3cc4ccccc4cc3-c3cccc4ccccc34)n2)cc1 ; c1ccc(-c2nc(-c3ccc4c(c3)c3ccccc3n4-c3ccccc3)nc(-c3cc4ccccc4cc3-c3cccc4ccccc34)n2)cc1 ; CC1(C)c2ccccc2-c2ccc(-c3nc(-c4ccccc4)nc(-c4cc5ccccc5cc4-c4cccc5ccccc45)n3)cc21]

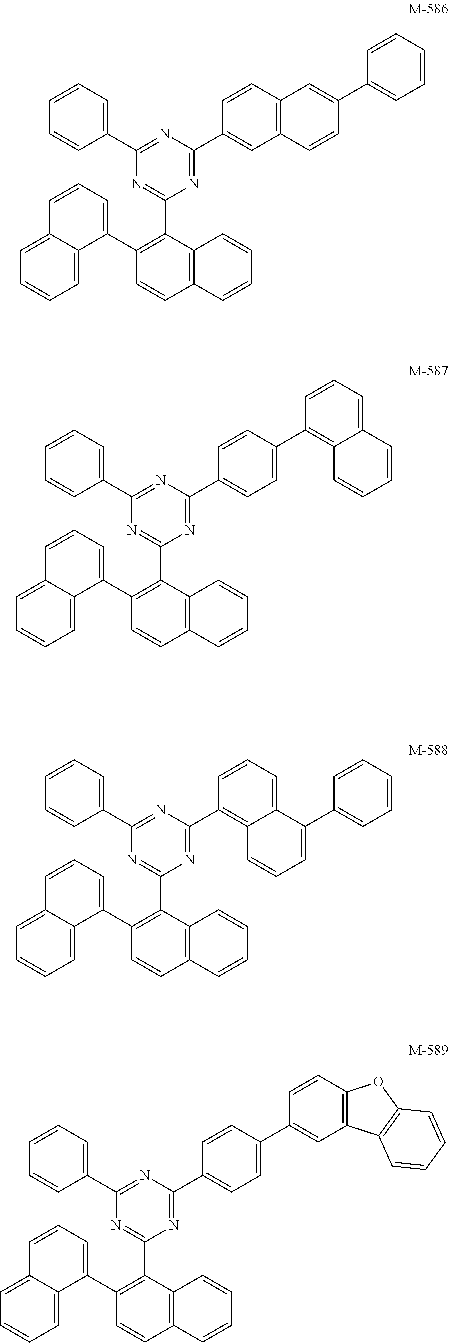 [SMILES: c1ccc(-c2ccc3cc(-c4nc(-c5ccccc5)nc(-c5c(-c6cccc7ccccc67)ccc6ccccc56)n4)ccc3c2)cc1 ; c1ccc(-c2nc(-c3ccc(-c4cccc5ccccc45)cc3)nc(-c3c(-c4cccc5ccccc45)ccc4ccccc34)n2)cc1 ; c1ccc(-c2nc(-c3c(-c4cccc5ccccc45)ccc4ccccc34)nc(-c3cccc4c(-c5ccccc5)cccc34)n2)cc1 ; c1ccc(-c2nc(-c3ccc(-c4ccc5oc6ccccc6c5c4)cc3)nc(-c3c(-c4cccc5ccccc45)ccc4ccccc34)n2)cc1]

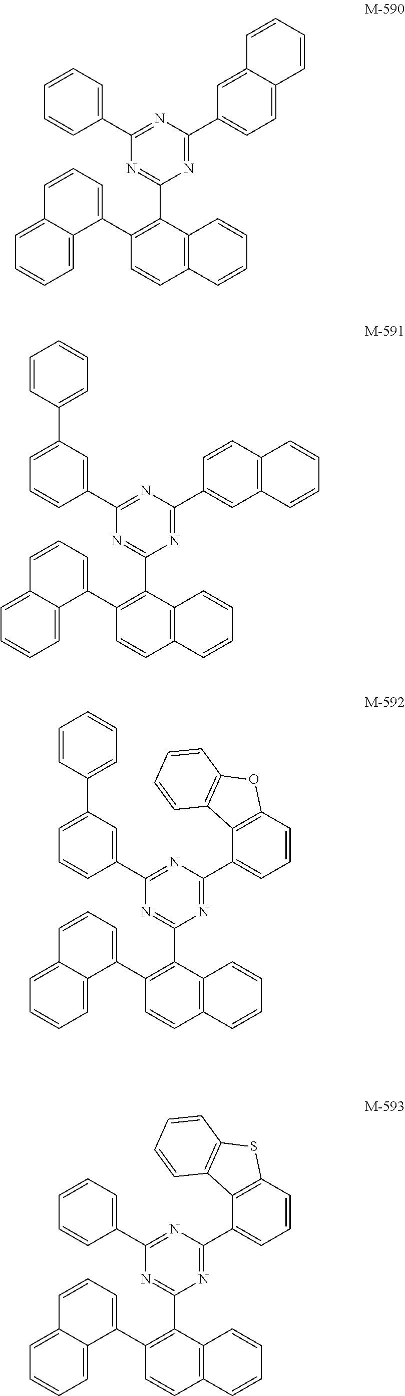 [SMILES: c1ccc(-c2nc(-c3ccc4ccccc4c3)nc(-c3c(-c4cccc5ccccc45)ccc4ccccc34)n2)cc1 ; c1ccc(-c2cccc(-c3nc(-c4ccc5ccccc5c4)nc(-c4c(-c5cccc6ccccc56)ccc5ccccc45)n3)c2)cc1 ; c1ccc(-c2cccc(-c3nc(-c4c(-c5cccc6ccccc56)ccc5ccccc45)nc(-c4cccc5oc6ccccc6c45)n3)c2)cc1 ; c1ccc(-c2nc(-c3c(-c4cccc5ccccc45)ccc4ccccc34)nc(-c3cccc4sc5ccccc5c34)n2)cc1]

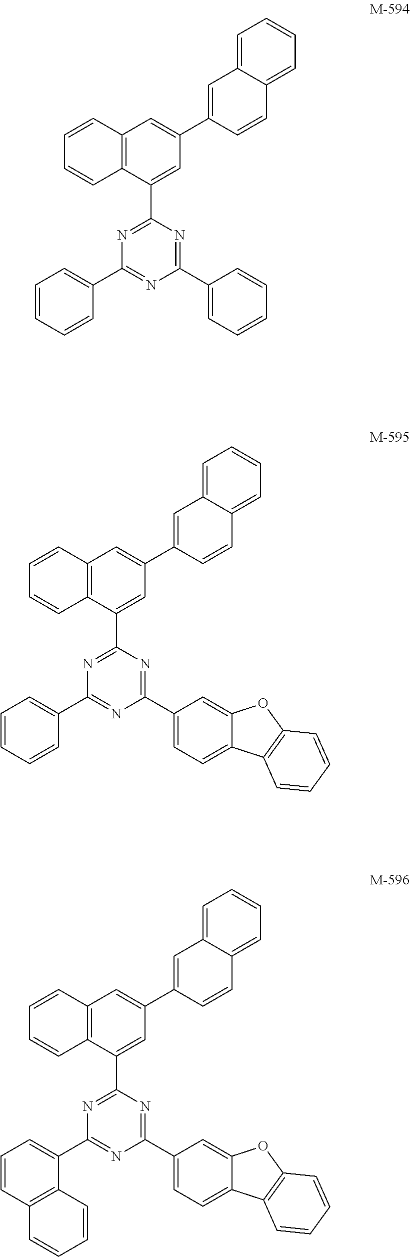 [SMILES: c1ccc(-c2nc(-c3ccccc3)nc(-c3cc(-c4ccc5ccccc5c4)cc4ccccc34)n2)cc1 ; c1ccc(-c2nc(-c3ccc4c(c3)oc3ccccc34)nc(-c3cc(-c4ccc5ccccc5c4)cc4ccccc34)n2)cc1 ; c1ccc2cc(-c3cc(-c4nc(-c5ccc6c(c5)oc5ccccc56)nc(-c5cccc6ccccc56)n4)c4ccccc4c3)ccc2c1]

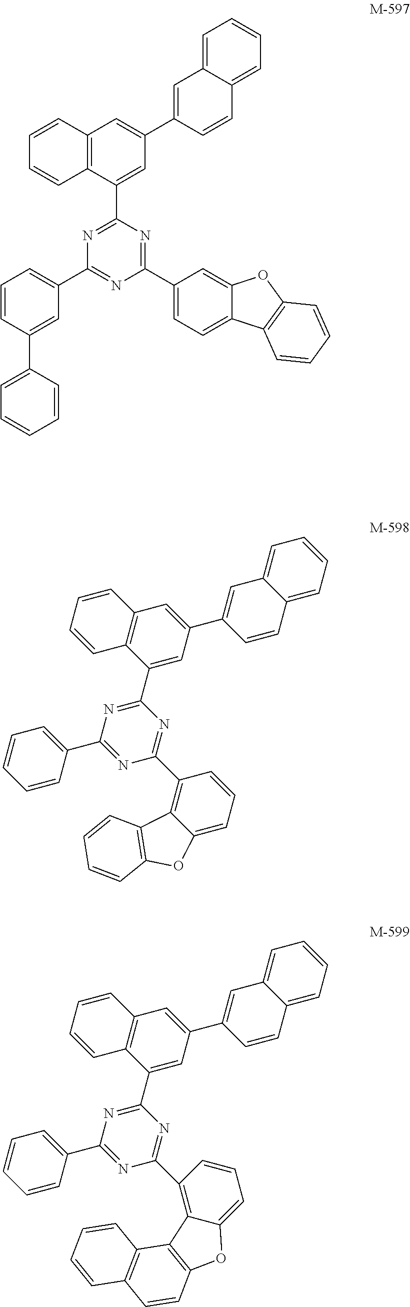 [SMILES: c1ccc(-c2cccc(-c3nc(-c4ccc5c(c4)oc4ccccc45)nc(-c4cc(-c5ccc6ccccc6c5)cc5ccccc45)n3)c2)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cc(-c4ccc5ccccc5c4)cc4ccccc34)nc(-c3cccc4oc5ccc6ccccc6c5c34)n2)cc1]

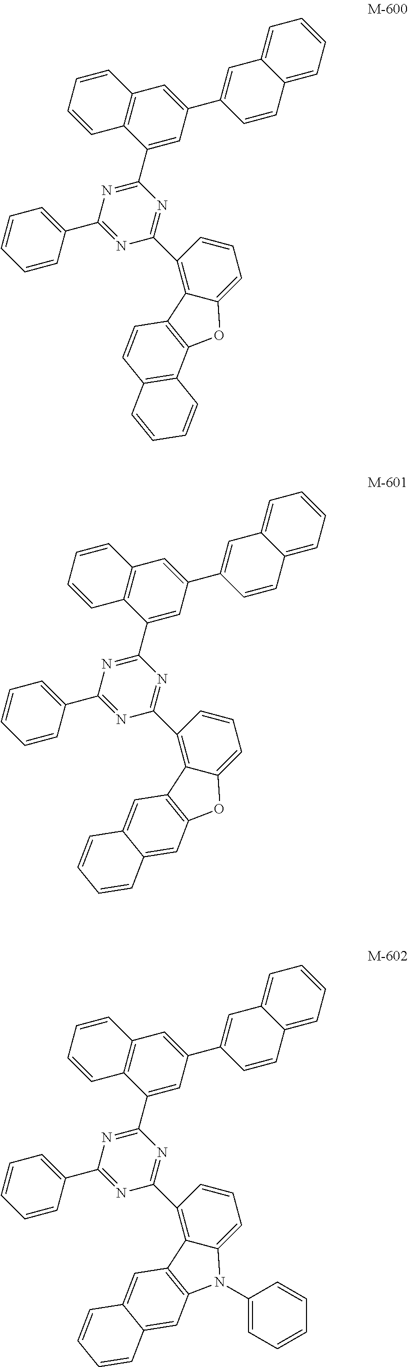 [SMILES: c1ccc(-c2nc(-c3cc(-c4ccc5ccccc5c4)cc4ccccc34)nc(-c3cccc4oc5c6ccccc6ccc5c34)n2)cc1 ; c1ccc(-c2nc(-c3cc(-c4ccc5ccccc5c4)cc4ccccc34)nc(-c3cccc4oc5cc6ccccc6cc5c34)n2)cc1 ; c1ccc(-c2nc(-c3cc(-c4ccc5ccccc5c4)cc4ccccc34)nc(-c3cccc4c3c3cc5ccccc5cc3n4-c3ccccc3)n2)cc1]

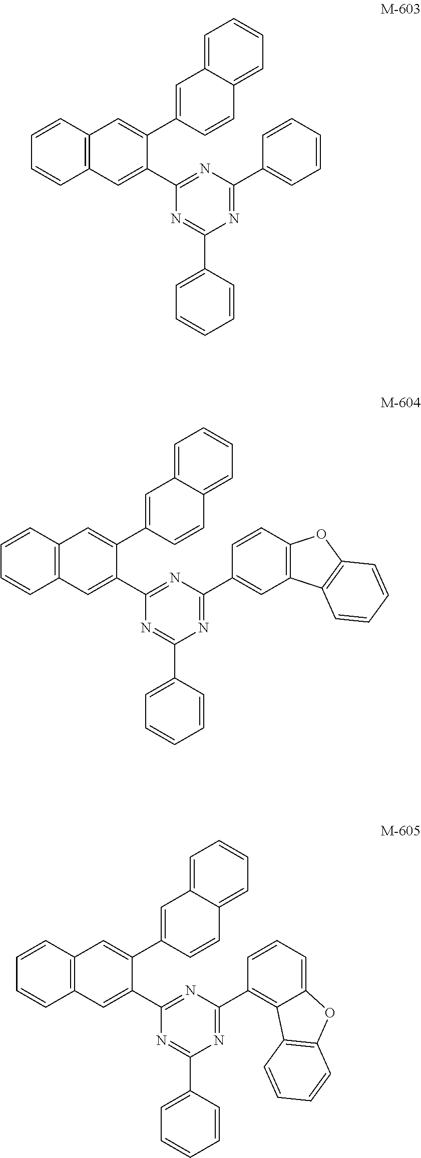 [SMILES: c1ccc(-c2nc(-c3ccccc3)nc(-c3cc4ccccc4cc3-c3ccc4ccccc4c3)n2)cc1 ; c1ccc(-c2nc(-c3ccc4oc5ccccc5c4c3)nc(-c3cc4ccccc4cc3-c3ccc4ccccc4c3)n2)cc1 ; c1ccc(-c2nc(-c3cc4ccccc4cc3-c3ccc4ccccc4c3)nc(-c3cccc4oc5ccccc5c34)n2)cc1]

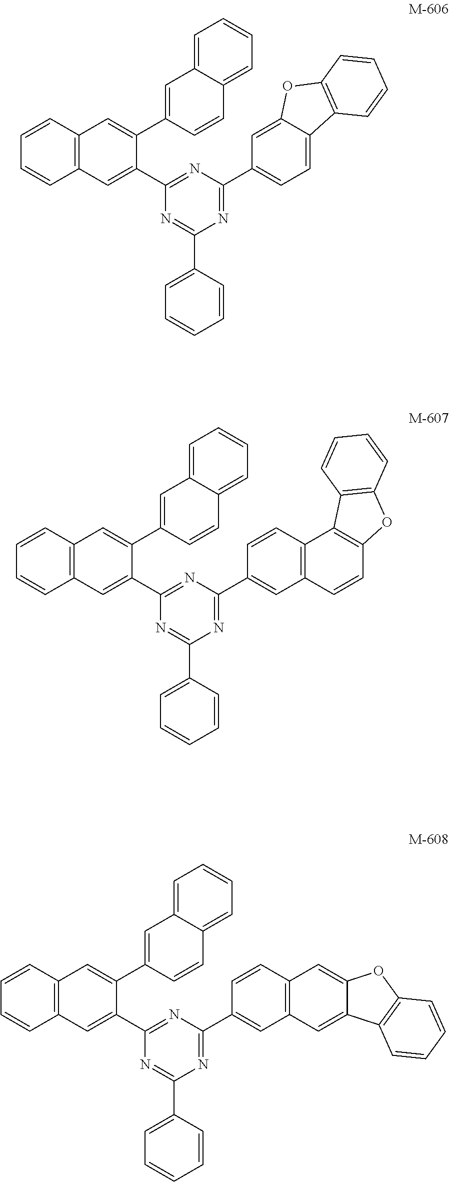 [SMILES: c1ccc(-c2nc(-c3ccc4c(c3)oc3ccccc34)nc(-c3cc4ccccc4cc3-c3ccc4ccccc4c3)n2)cc1 ; c1ccc(-c2nc(-c3ccc4c(ccc5oc6ccccc6c54)c3)nc(-c3cc4ccccc4cc3-c3ccc4ccccc4c3)n2)cc1 ; c1ccc(-c2nc(-c3ccc4cc5oc6ccccc6c5cc4c3)nc(-c3cc4ccccc4cc3-c3ccc4ccccc4c3)n2)cc1]

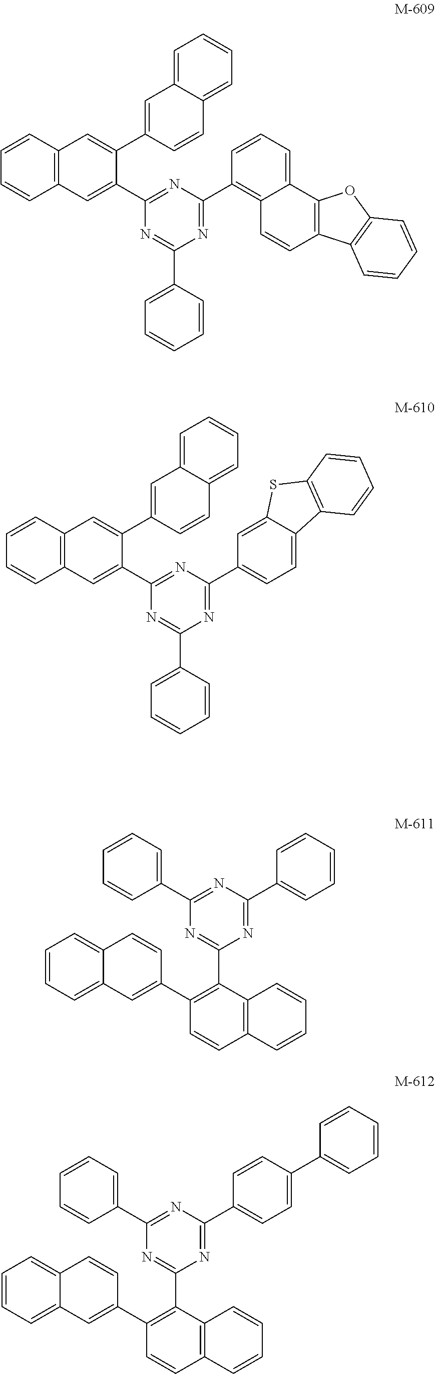 [SMILES: c1ccc(-c2nc(-c3cc4ccccc4cc3-c3ccc4ccccc4c3)nc(-c3cccc4c3ccc3c5ccccc5oc43)n2)cc1 ; c1ccc(-c2nc(-c3ccc4c(c3)sc3ccccc34)nc(-c3cc4ccccc4cc3-c3ccc4ccccc4c3)n2)cc1 ; c1ccc(-c2nc(-c3ccccc3)nc(-c3c(-c4ccc5ccccc5c4)ccc4ccccc34)n2)cc1 ; c1ccc(-c2ccc(-c3nc(-c4ccccc4)nc(-c4c(-c5ccc6ccccc6c5)ccc5ccccc45)n3)cc2)cc1]

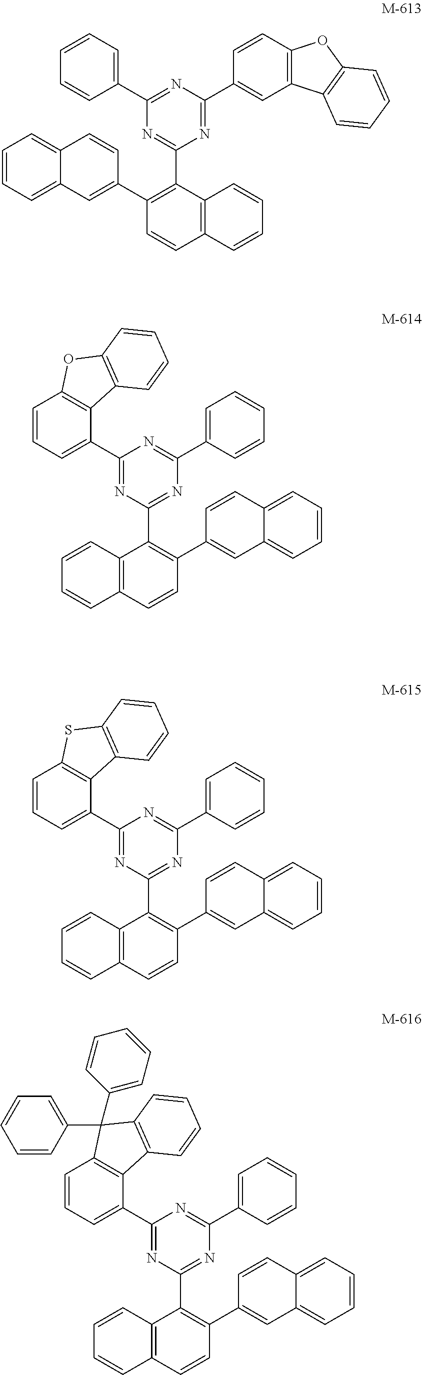 [SMILES: c1ccc(-c2nc(-c3ccc4oc5ccccc5c4c3)nc(-c3c(-c4ccc5ccccc5c4)ccc4ccccc34)n2)cc1 ; c1ccc(-c2nc(-c3c(-c4ccc5ccccc5c4)ccc4ccccc34)nc(-c3cccc4oc5ccccc5c34)n2)cc1 ; c1ccc(-c2nc(-c3c(-c4ccc5ccccc5c4)ccc4ccccc34)nc(-c3cccc4sc5ccccc5c34)n2)cc1 ; c1ccc(-c2nc(-c3cccc4c3-c3ccccc3C4(c3ccccc3)c3ccccc3)nc(-c3c(-c4ccc5ccccc5c4)ccc4ccccc34)n2)cc1]

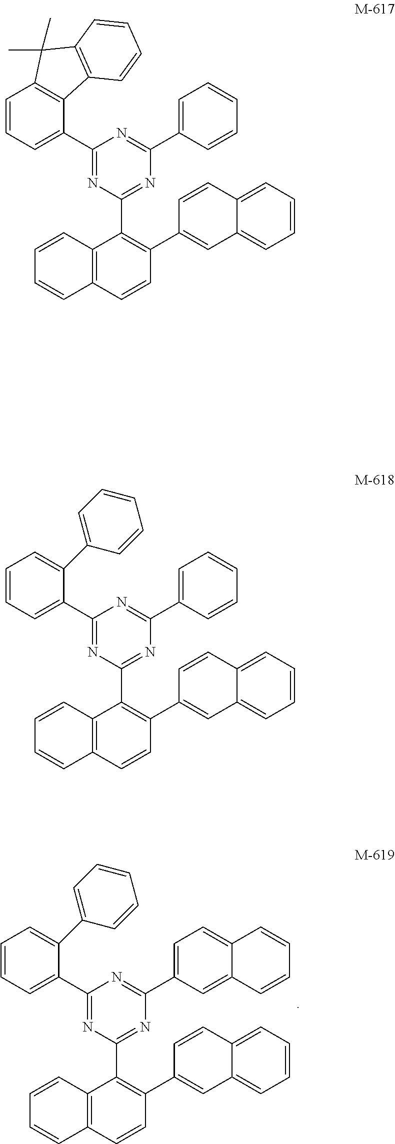 [SMILES: CC1(C)c2ccccc2-c2c(-c3nc(-c4ccccc4)nc(-c4c(-c5ccc6ccccc6c5)ccc5ccccc45)n3)cccc21 ; c1ccc(-c2nc(-c3ccccc3-c3ccccc3)nc(-c3c(-c4ccc5ccccc5c4)ccc4ccccc34)n2)cc1 ; c1ccc(-c2ccccc2-c2nc(-c3ccc4ccccc4c3)nc(-c3c(-c4ccc5ccccc5c4)ccc4ccccc34)n2)cc1]

An organic electroluminescent host material composition includes a compound N having a structure shown in Formula (1) and the compound M having the structure shown in Formula (2) above;

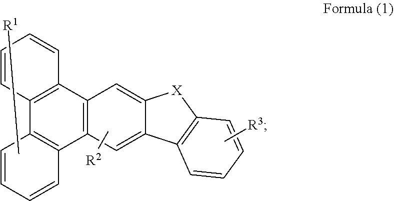

in Formula (1), X is selected from O, S, Se, NAr and CR⁶R⁷; wherein Ar is selected from hydrogen, deuterium, halogen, cyano, substituted or unsubstituted C1-C30 alkyl, substituted or unsubstituted C3-C30 cycloalkyl, substituted or unsubstituted C5-C60 aryl, and substituted or unsubstituted C3-C60 heteroaryl; R⁶-R⁷ are each independently selected from hydrogen atoms, substituted or unsubstituted C1-C20 alkyl, substituted or unsubstituted C6-C30 aryl, and substituted or unsubstituted C3-C30 heteroaryl;
R¹ is -L¹Ar¹, R² is -L²Ar², and R³ is -L³Ar³; L¹-L³ are each independently selected from a connecting bond, substituted or unsubstituted C6-C30 arylenyl, and substituted or unsubstituted C3-C30 heteroarylenyl; at least one of Ar¹-Ar³ is

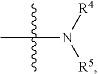

[symbol]

represents a connecting bond, R⁴ is -L⁴Ar⁴, and R⁵ is -L⁵Ar⁵; L⁴-L⁵ are each independently selected from a connecting bond, substituted or unsubstituted C6-C30 arylenyl and substituted or unsubstituted C3-C30 heteroarylenyl, and Ar⁴-Ar⁵ are each independently selected from hydrogen, substituted or unsubstituted C6-C30 aryl and substituted or unsubstituted C3-C30 heteroaryl; and the remaining of Ar¹-Ar³ are each independently selected from hydrogen, deuterium, tritium, halogen, cyano, substituted or unsubstituted C6-C60 arylamine group, substituted or unsubstituted C3-C60 heteroarylamine group, substituted or unsubstituted C6-C60 aryl, and substituted or unsubstituted C3-C60 heteroaryl.

In Formula (1)

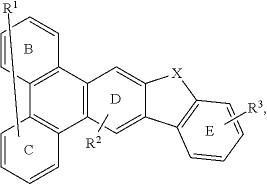

it is to be understood that in the present disclosure, R¹ may be substituted on a ring B or a ring C, R² may be substituted on a ring D, and R³ may be substituted on a ring E.

Preferably, Ar⁴-Ar⁵ are each independently selected from the following substituted or unsubstituted groups: phenyl, naphthyl, biphenyl, triphenyl, phenanthryl, triphenylenyl, chrysenyl, dibenzofuryl, benzonaphthofuryl, dibenzothienyl, dibenzoselenophenyl, triphenylenyl, dimethylfluorenyl, spirobifluorenyl, fluoranthenyl, carbazolyl, phenylcarbazolyl, diphenylfluorenyl, benzodimethylfluorenyl, benzodiphenylfluorenyl, benzospirobifluorenyl, pyridyl, pyrimidinyl and triazinyl.

Ar⁴-Ar⁵ are each independently selected from

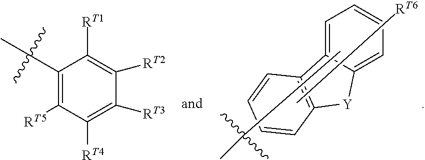

R^{T1}-R^{T6} are each independently selected from hydrogen, deuterium, halogen, cyano, substituted or unsubstituted C1-C30 alkyl, substituted or unsubstituted C7-C30 aralkyl, substituted or unsubstituted C6-C30 aryl, substituted or unsubstituted C3-C30 heteroaryl, substituted or unsubstituted C4-C30 heteroaralkyl, substituted or unsubstituted C3-C30 cycloalkyl, substituted or unsubstituted C3-C30 heterocycloalkyl, substituted or unsubstituted C3-C30 cycloalkenyl, substituted or unsubstituted C1-C30 alkoxy, and substituted or unsubstituted C6-C30 aryloxy, or any two adjacent ones of R^{T1}-R^{T5} are capable of being fused to a C6-C30 ring A; Y is selected from O, S, Nar and CR⁶R⁷; wherein Ar, R⁶ and R⁷ are defined the same as the above;
when a plurality of R^{T1}-R^{T6} are present, R^{T1}-R^{T6} are independent of each other and may be the same or different; and
preferably, ring A is selected from a substituted or unsubstituted benzene ring, a substituted or unsubstituted naphthalene ring, and a substituted or unsubstituted phenanthrene ring.
Ar is selected from substituted or unsubstituted C6-C30 aryl and C3-C30 heteroaryl; preferably, Ar is selected from the following substituted or unsubstituted groups: phenyl, naphthyl, biphenyl, triphenyl, triphenylenyl, chrysenyl, dibenzofuryl and dibenzothienyl;
and/or, R⁶ and R⁷ are each independently selected from substituted or unsubstituted C1-C5 alkyl and substituted or unsubstituted C6-C30 aryl.
L¹-L³ are each independently selected from a connecting bond and C6-C30 arylenyl, and preferably, L¹-L³ are each independently selected from a connecting bond, phenylenyl, naphthylenyl, triphenylenyl and biphenylenyl; further optionally, L¹ is selected from a connecting bond, L² is selected from phenylenyl, naphthylenyl, triphenylenyl and biphenylenyl, and L³ is a connecting bond; preferably, L¹-L³ are each independently selected from a single bond;
and/or, L⁴-L⁵ are each independently selected from a single bond and substituted or unsubstituted C6-C30 arylenyl; optionally, L⁴-L⁵ are each independently selected from a single bond, phenylenyl and naphthylenyl, and further optionally, L⁴-L⁵ are each independently selected from a single bond;
and/or, the remaining of Ar¹-Ar³ are selected from hydrogen, deuterium, halogen, cyano, substituted or unsubstituted C6-C30 aryl, substituted or unsubstituted C3-C30 heteroaryl, substituted or unsubstituted dibenzofuryl, and substituted or unsubstituted dibenzothienyl.

The structure of the compound N is as shown in any one of Formula 1-1 to Formula 1-17;

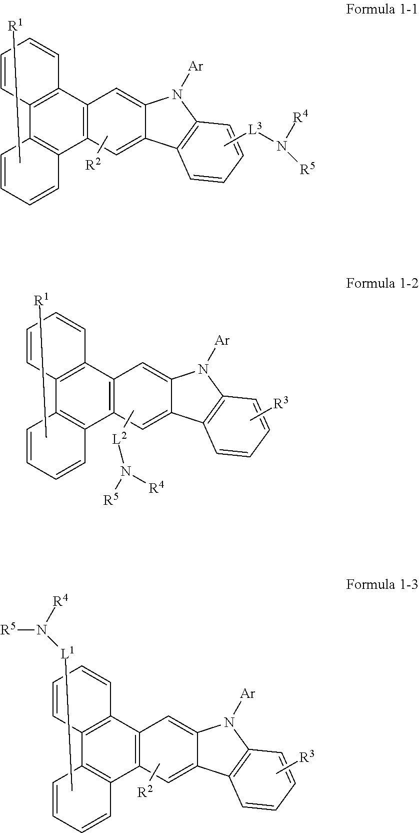

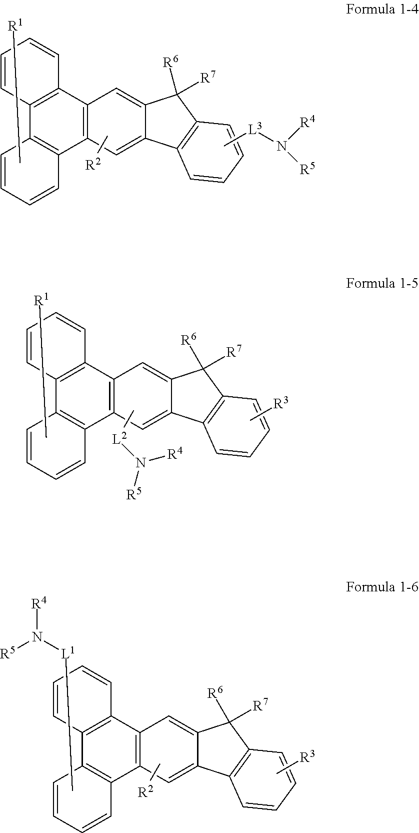

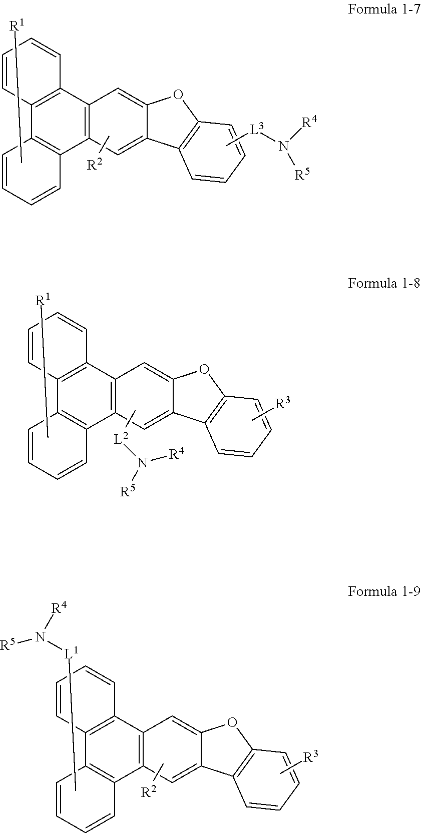

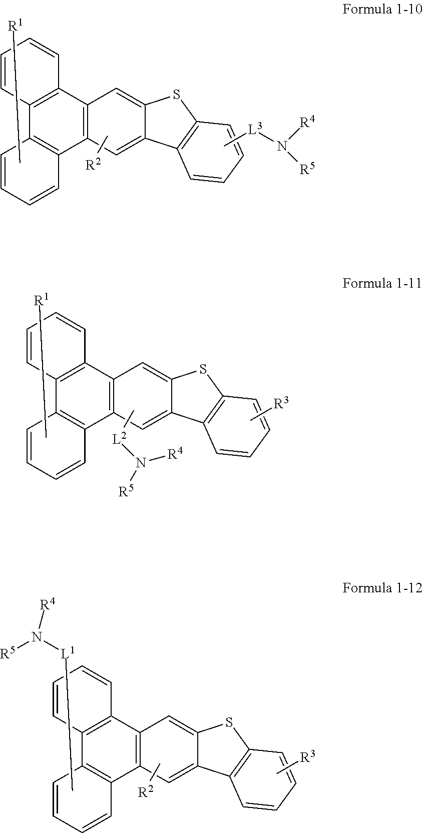

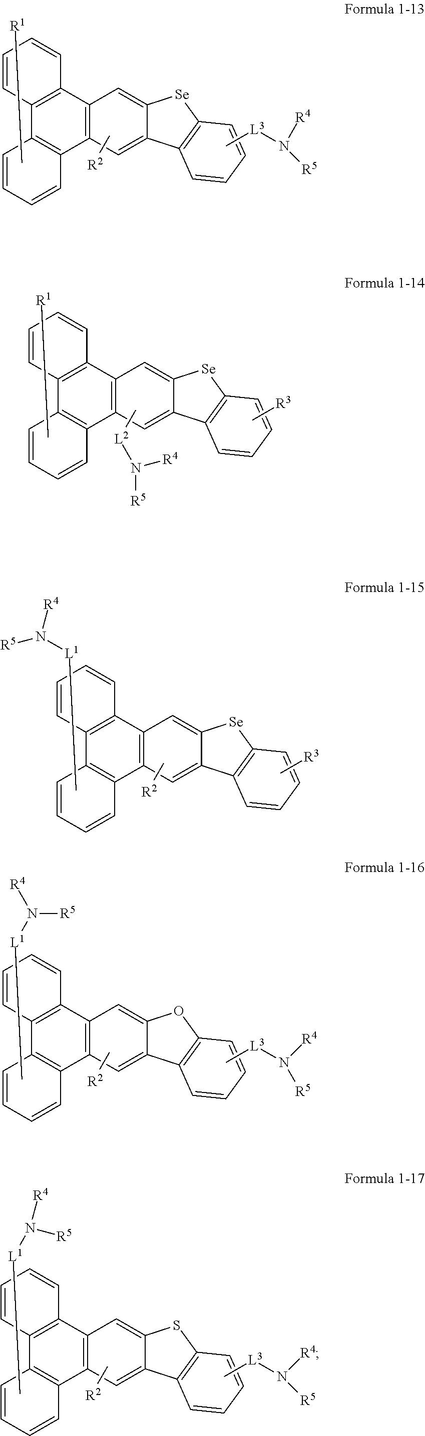

wherein R¹-R⁷, L¹-L³ and Ar are defined the same as the above.

The structure of the compound N shown in Formula (1) is as shown in any one of N-1 to N-935.

The structures of N-1 to N-935 are as follows:

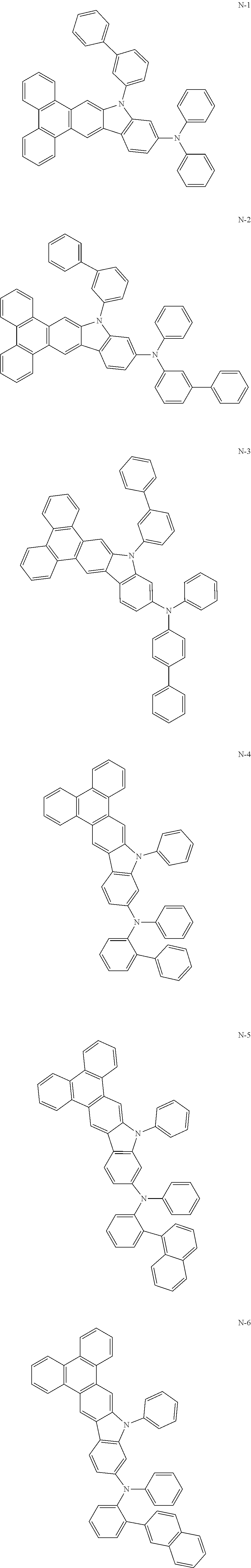

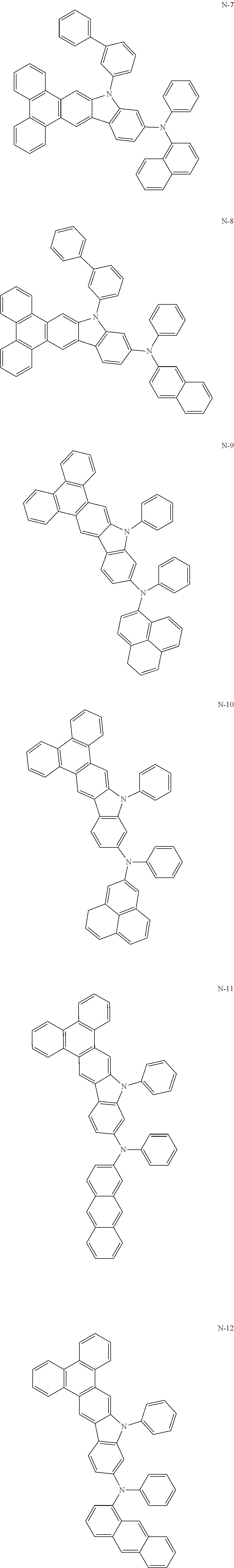

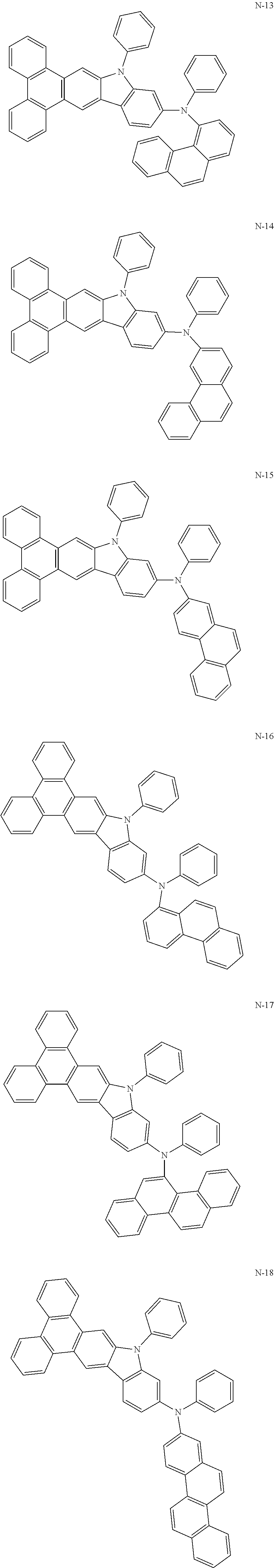

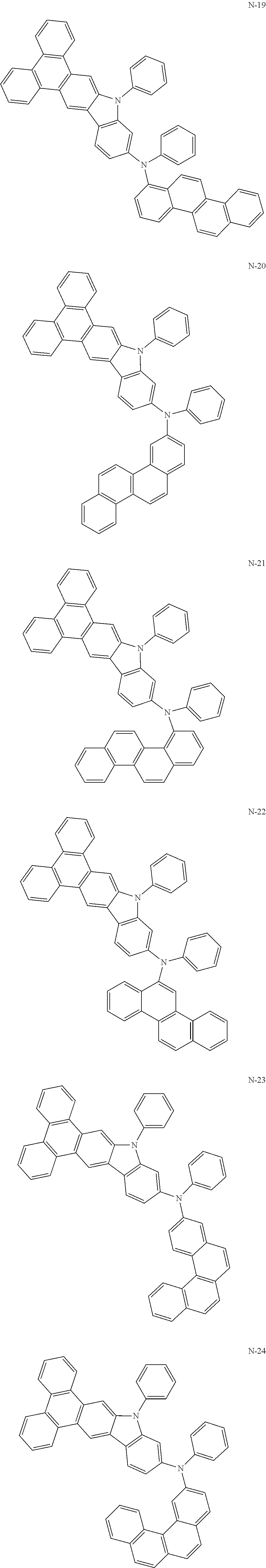

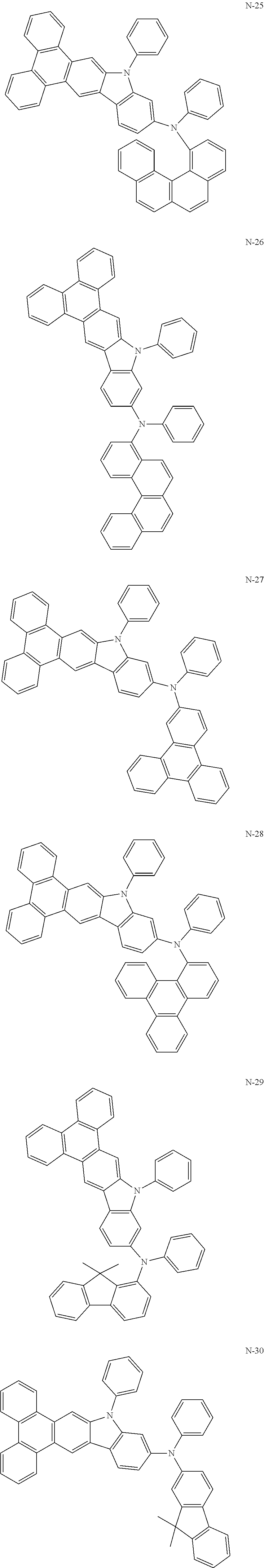

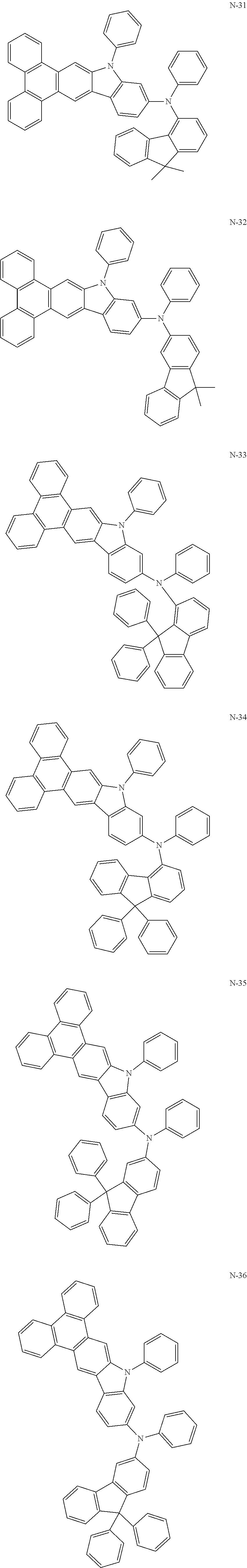

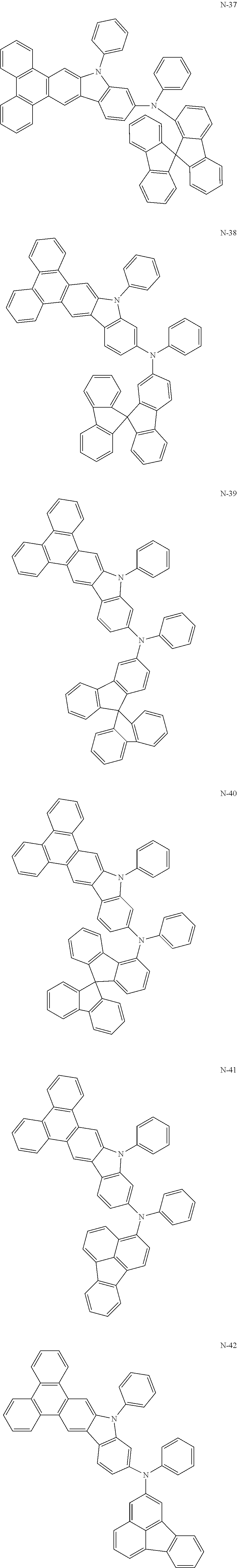

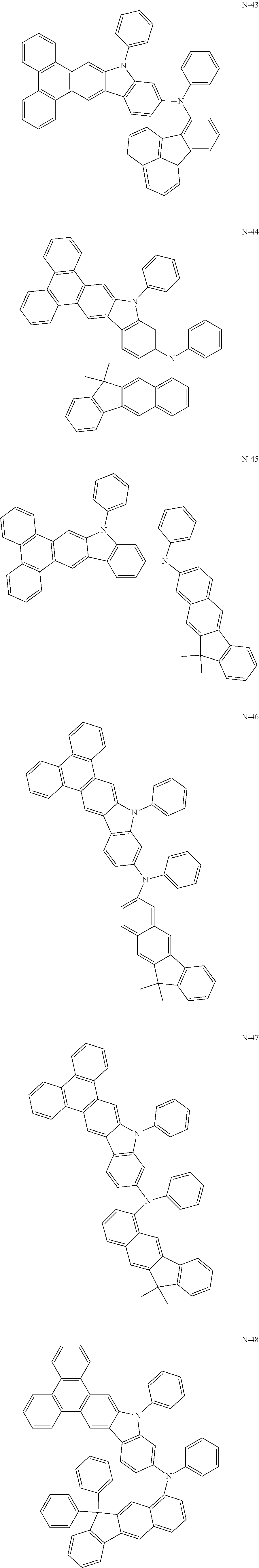

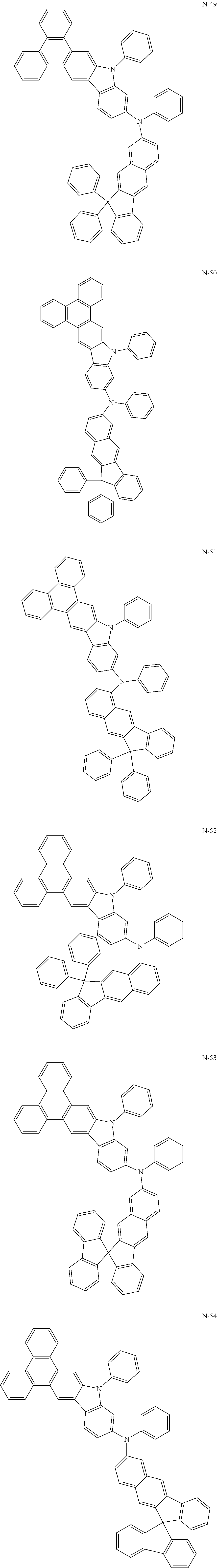

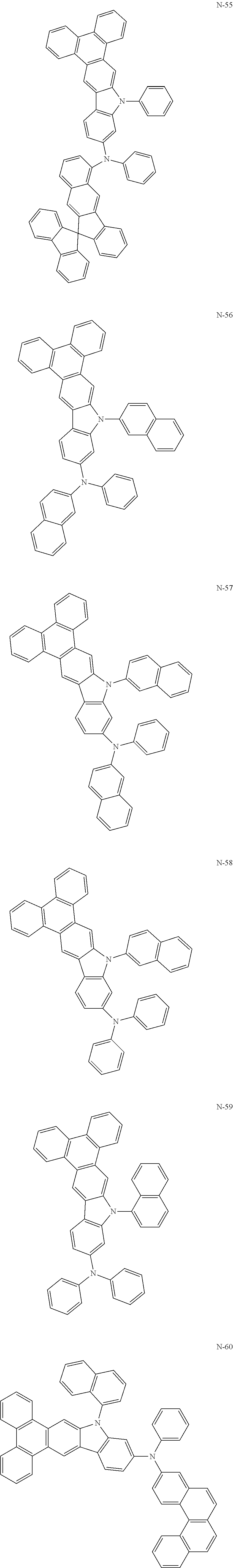

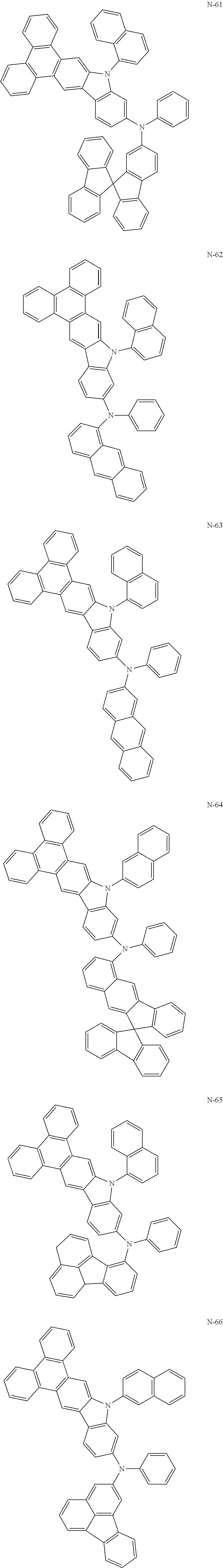

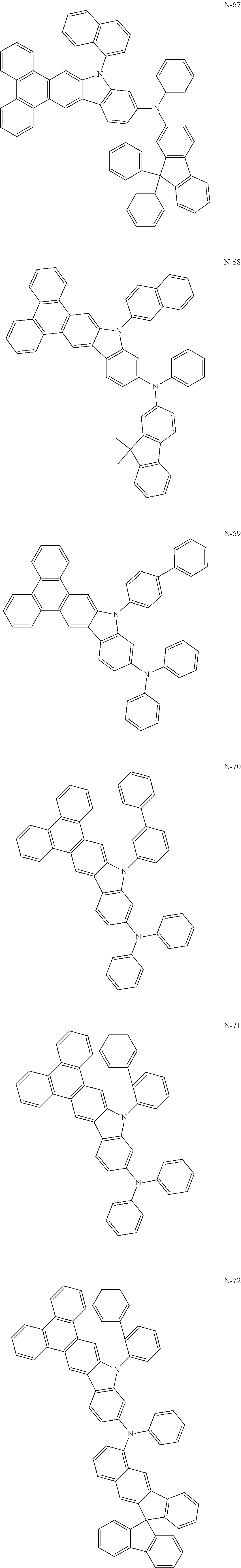

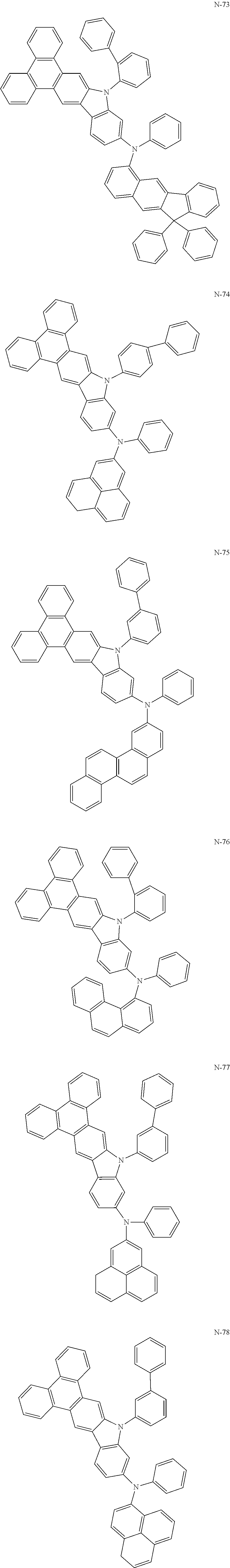

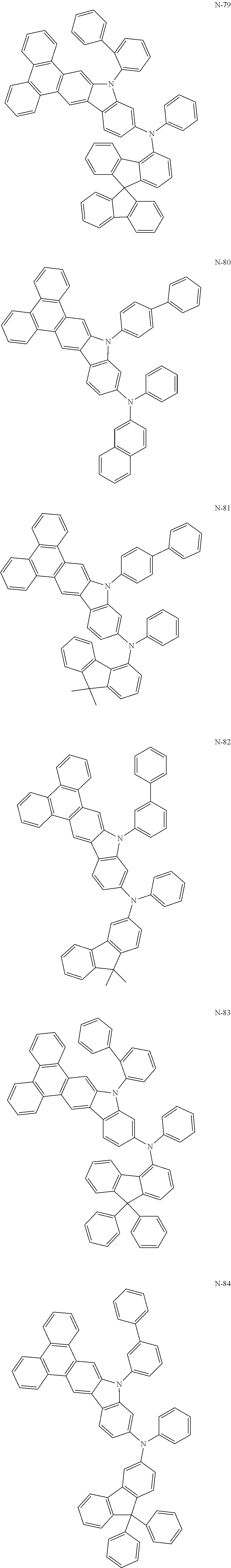

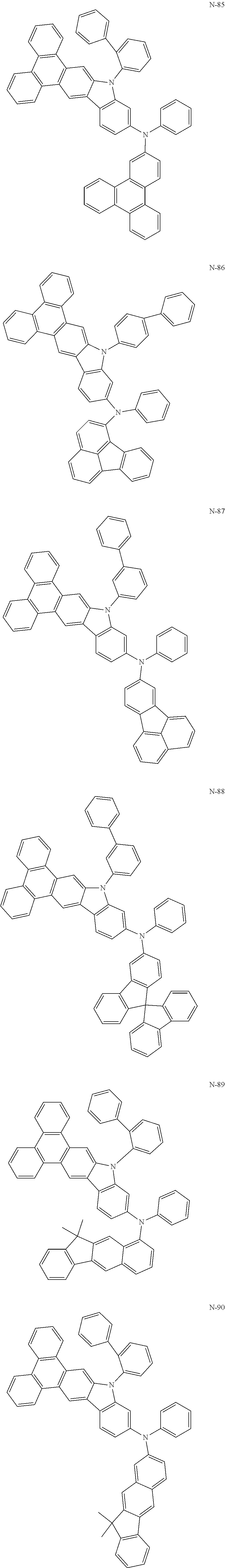

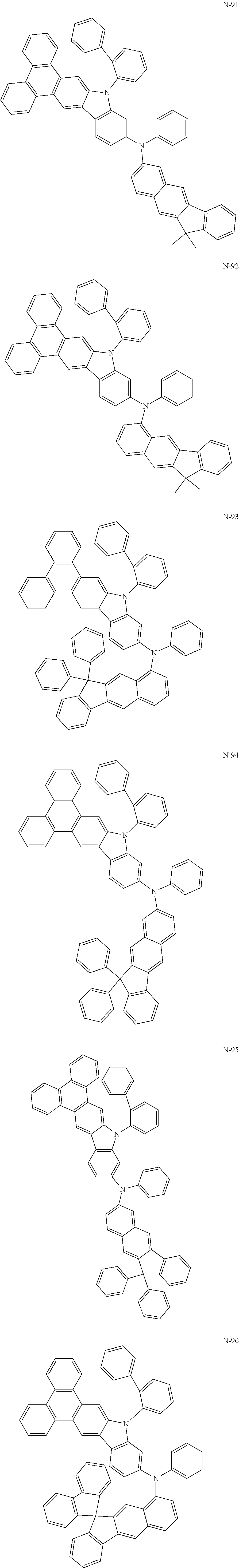

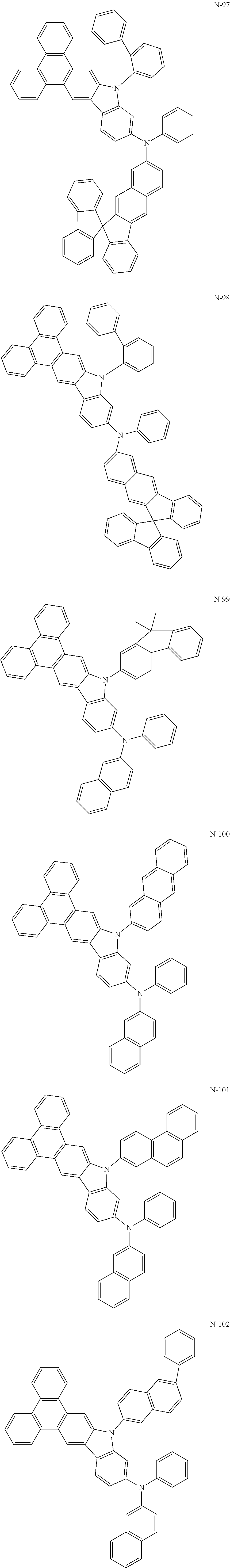

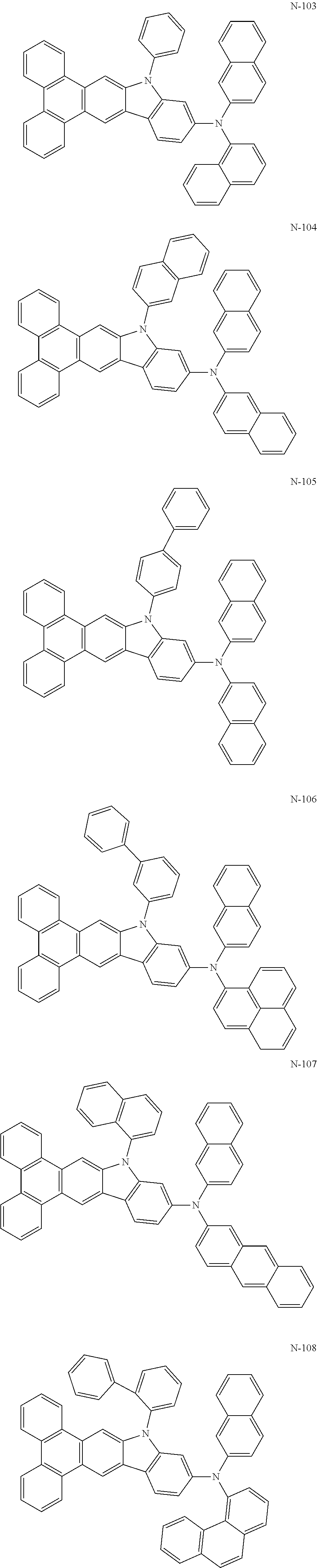

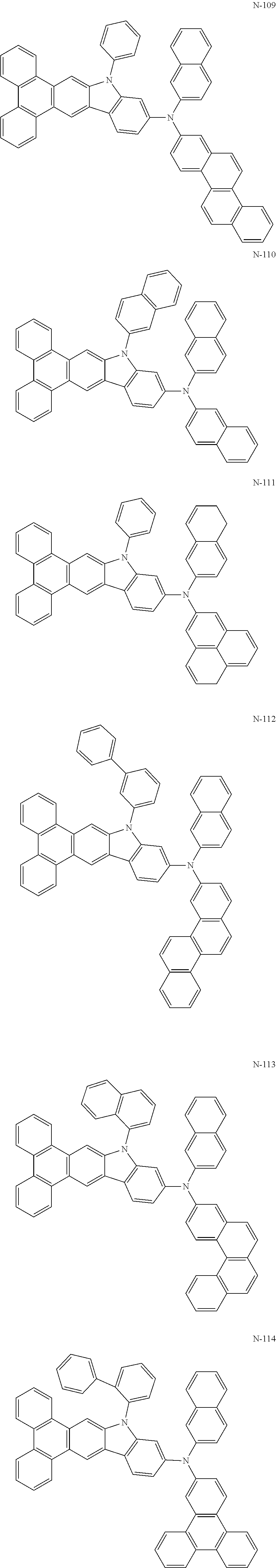

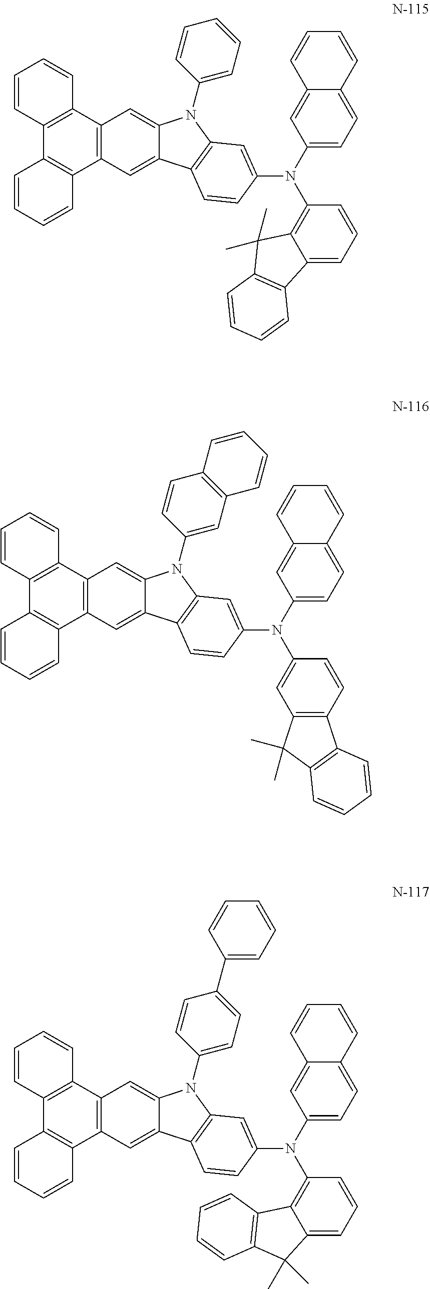 [SMILES: CC1(C)c2ccccc2-c2cccc(N(c3ccc4ccccc4c3)c3ccc4c5cc6c7ccccc7c7ccccc7c6cc5n(-c5ccccc5)c4c3)c21 ; CC1(C)c2ccccc2-c2ccc(N(c3ccc4ccccc4c3)c3ccc4c5cc6c7ccccc7c7ccccc7c6cc5n(-c5ccc6ccccc6c5)c4c3)cc21 ; CC1(C)c2ccccc2-c2c(N(c3ccc4ccccc4c3)c3ccc4c5cc6c7ccccc7c7ccccc7c6cc5n(-c5ccc(-c6ccccc6)cc5)c4c3)cccc21]

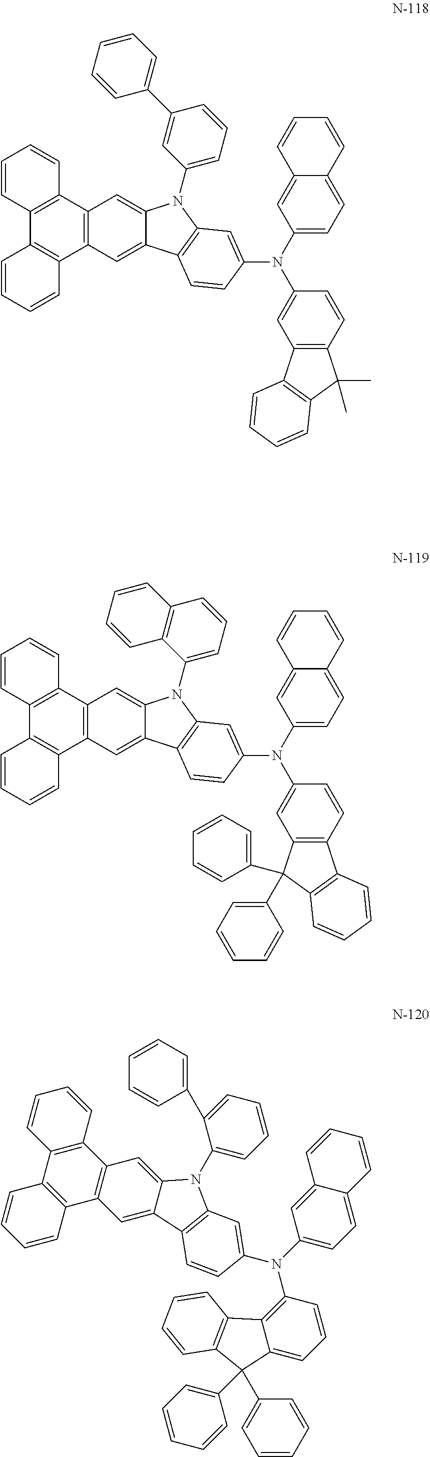 [SMILES: CC1(C)c2ccccc2-c2cc(N(c3ccc4ccccc4c3)c3ccc4c5cc6c7ccccc7c7ccccc7c6cc5n(-c5cccc(-c6ccccc6)c5)c4c3)ccc21 ; c1ccc(C2(c3ccccc3)c3ccccc3-c3ccc(N(c4ccc5ccccc5c4)c4ccc5c6cc7c8ccccc8c8ccccc8c7cc6n(-c6cccc7ccccc67)c5c4)cc32)cc1 ; c1ccc(-c2ccccc2-n2c3cc(N(c4ccc5ccccc5c4)c4cccc5c4-c4ccccc4C5(c4ccccc4)c4ccccc4)ccc3c3cc4c5ccccc5c5ccccc5c4cc32)cc1]

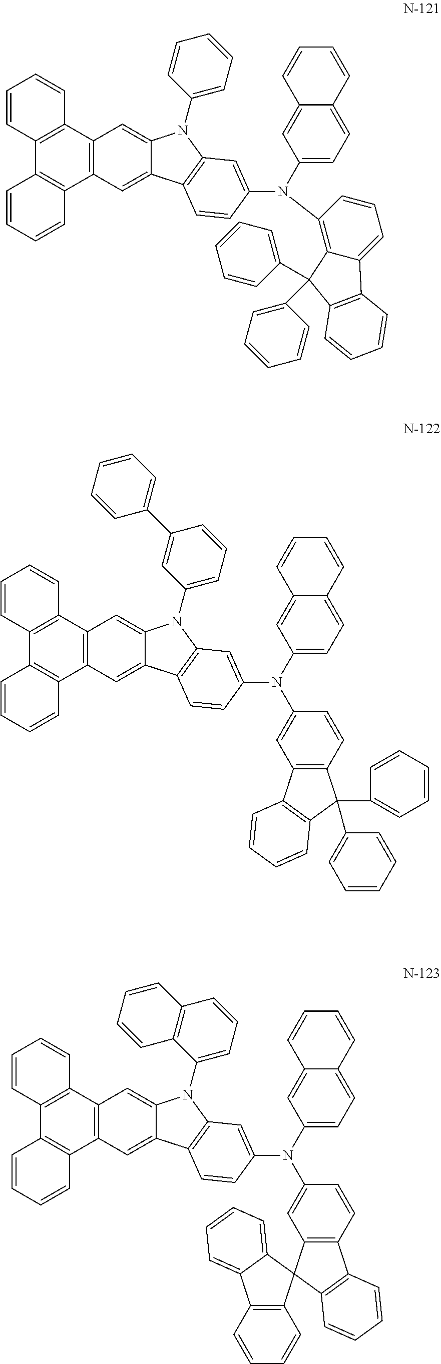 [SMILES: c1ccc(-n2c3cc(N(c4ccc5ccccc5c4)c4cccc5c4C(c4ccccc4)(c4ccccc4)c4ccccc4-5)ccc3c3cc4c5ccccc5c5ccccc5c4cc32)cc1 ; c1ccc(-c2cccc(-n3c4cc(N(c5ccc6c(c5)-c5ccccc5C6(c5ccccc5)c5ccccc5)c5ccc6ccccc6c5)ccc4c4cc5c6ccccc6c6ccccc6c5cc43)c2)cc1 ; c1ccc2c(c1)-c1ccccc1C21c2ccccc2-c2ccc(N(c3ccc4ccccc4c3)c3ccc4c5cc6c7ccccc7c7ccccc7c6cc5n(-c5cccc6ccccc56)c4c3)cc21]

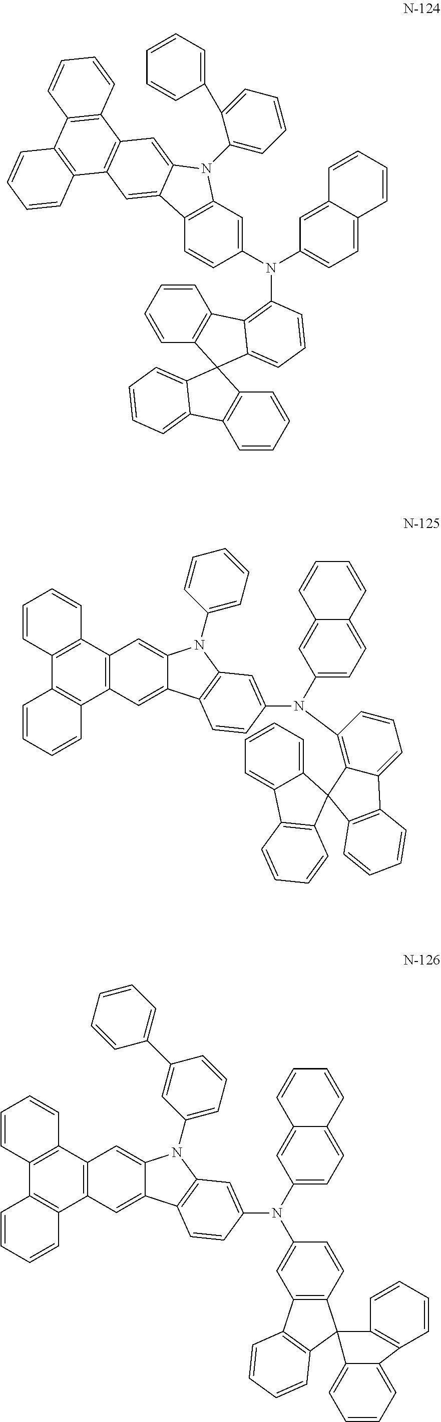 [SMILES: c1ccc(-c2ccccc2-n2c3cc(N(c4ccc5ccccc5c4)c4cccc5c4-c4ccccc4C54c5ccccc5-c5ccccc54)ccc3c3cc4c5ccccc5c5ccccc5c4cc32)cc1 ; c1ccc(-n2c3cc(N(c4ccc5ccccc5c4)c4cccc5c4C4(c6ccccc6-c6ccccc64)c4ccccc4-5)ccc3c3cc4c5ccccc5c5ccccc5c4cc32)cc1 ; c1ccc(-c2cccc(-n3c4cc(N(c5ccc6c(c5)-c5ccccc5C65c6ccccc6-c6ccccc65)c5ccc6ccccc6c5)ccc4c4cc5c6ccccc6c6ccccc6c5cc43)c2)cc1]

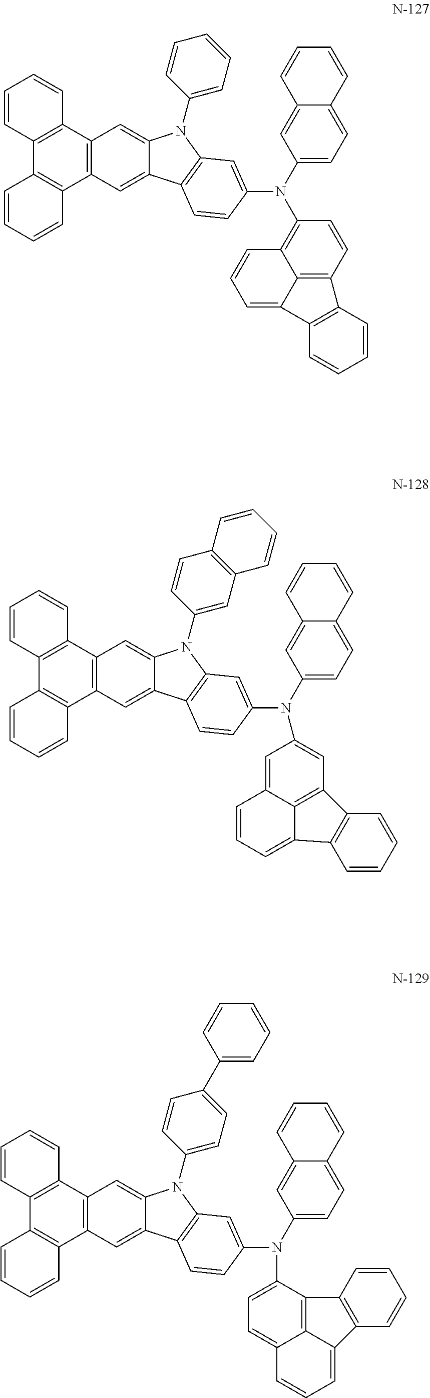 [SMILES: c1ccc(-n2c3cc(N(c4ccc5ccccc5c4)c4ccc5c6c(cccc46)-c4ccccc4-5)ccc3c3cc4c5ccccc5c5ccccc5c4cc32)cc1 ; c1ccc2c(c1)-c1cccc3cc(N(c4ccc5ccccc5c4)c4ccc5c6cc7c8ccccc8c8ccccc8c7cc6n(-c6ccc7ccccc7c6)c5c4)cc-2c13 ; c1ccc(-c2ccc(-n3c4cc(N(c5ccc6ccccc6c5)c5ccc6cccc7c6c5-c5ccccc5-7)ccc4c4cc5c6ccccc6c6ccccc6c5cc43)cc2)cc1]

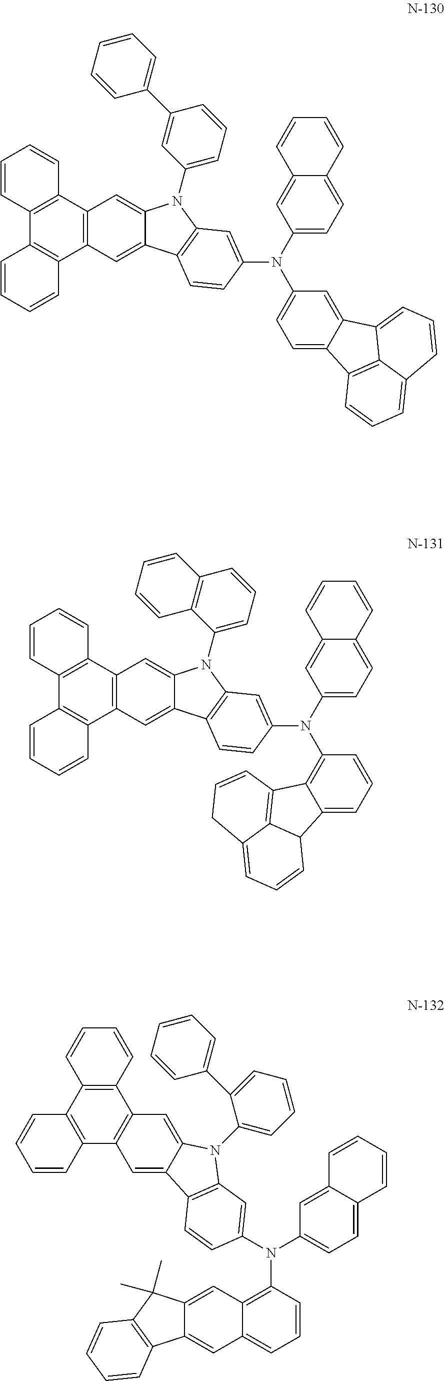 [SMILES: c1ccc(-c2cccc(-n3c4cc(N(c5ccc6c(c5)-c5cccc7cccc-6c57)c5ccc6ccccc6c5)ccc4c4cc5c6ccccc6c6ccccc6c5cc43)c2)cc1 ; C1=CC2C3=C(C=CCC3=C1)c1c2cccc1N(c1ccc2ccccc2c1)c1ccc2c3cc4c5ccccc5c5ccccc5c4cc3n(-c3cccc4ccccc34)c2c1 ; CC1(C)c2ccccc2-c2cc3cccc(N(c4ccc5ccccc5c4)c4ccc5c6cc7c8ccccc8c8ccccc8c7cc6n(-c6ccccc6-c6ccccc6)c5c4)c3cc21]

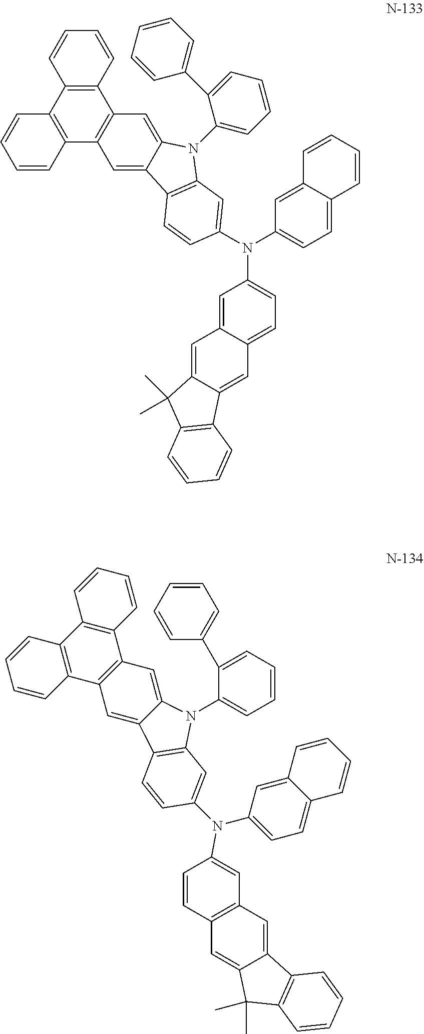 [SMILES: CC1(C)c2ccccc2-c2cc3ccc(N(c4ccc5ccccc5c4)c4ccc5c6cc7c8ccccc8c8ccccc8c7cc6n(-c6ccccc6-c6ccccc6)c5c4)cc3cc21 ; CC1(C)c2ccccc2-c2cc3cc(N(c4ccc5ccccc5c4)c4ccc5c6cc7c8ccccc8c8ccccc8c7cc6n(-c6ccccc6-c6ccccc6)c5c4)ccc3cc21]

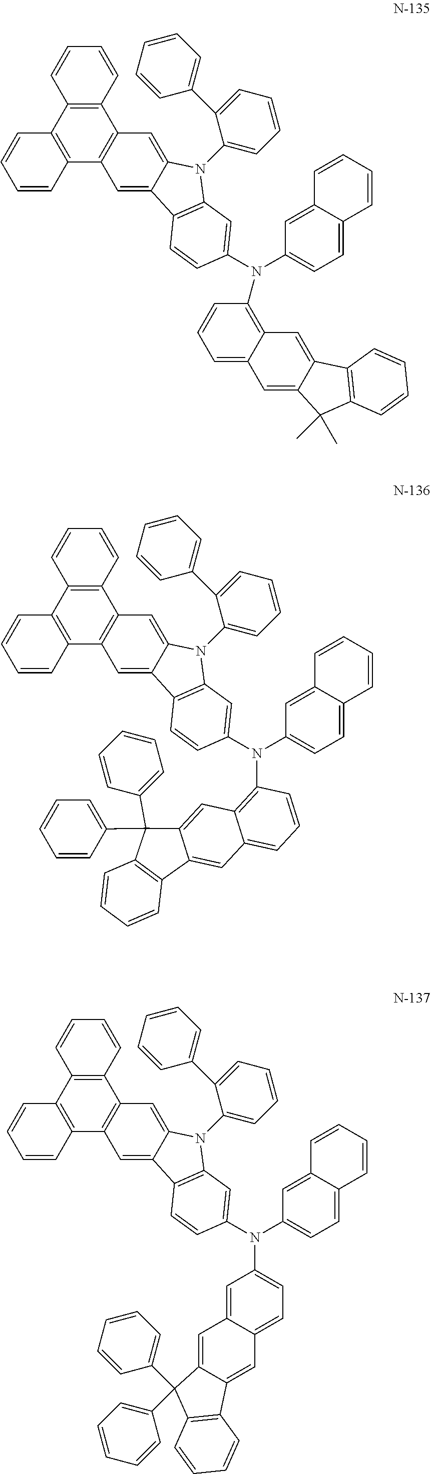 [SMILES: CC1(C)c2ccccc2-c2cc3c(N(c4ccc5ccccc5c4)c4ccc5c6cc7c8ccccc8c8ccccc8c7cc6n(-c6ccccc6-c6ccccc6)c5c4)cccc3cc21 ; c1ccc(-c2ccccc2-n2c3cc(N(c4ccc5ccccc5c4)c4cccc5cc6c(cc45)C(c4ccccc4)(c4ccccc4)c4ccccc4-6)ccc3c3cc4c5ccccc5c5ccccc5c4cc32)cc1 ; c1ccc(-c2ccccc2-n2c3cc(N(c4ccc5ccccc5c4)c4ccc5cc6c(cc5c4)C(c4ccccc4)(c4ccccc4)c4ccccc4-6)ccc3c3cc4c5ccccc5c5ccccc5c4cc32)cc1]

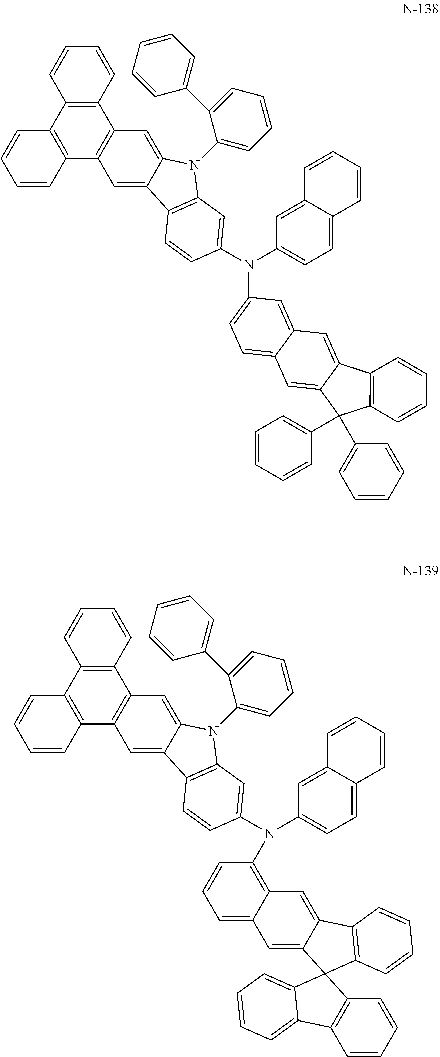 [SMILES: c1ccc(-c2ccccc2-n2c3cc(N(c4ccc5ccccc5c4)c4ccc5cc6c(cc5c4)-c4ccccc4C6(c4ccccc4)c4ccccc4)ccc3c3cc4c5ccccc5c5ccccc5c4cc32)cc1 ; c1ccc(-c2ccccc2-n2c3cc(N(c4ccc5ccccc5c4)c4cccc5cc6c(cc45)-c4ccccc4C64c5ccccc5-c5ccccc54)ccc3c3cc4c5ccccc5c5ccccc5c4cc32)cc1]

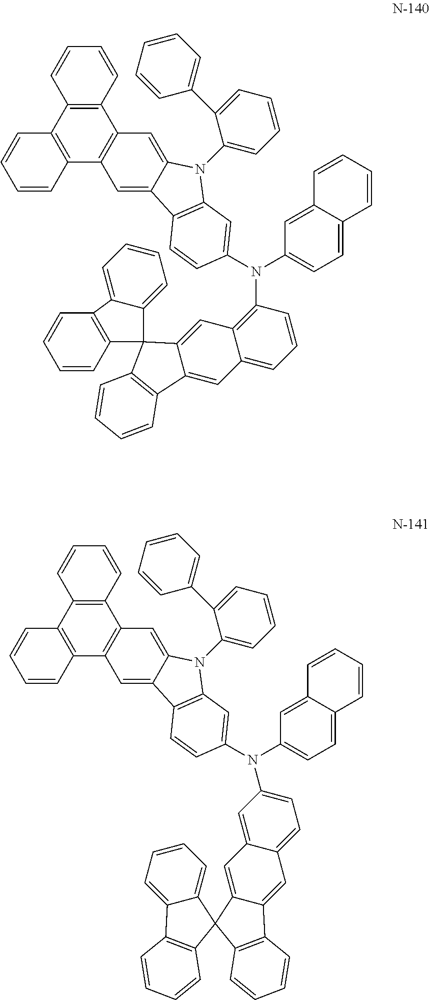 [SMILES: c1ccc(-c2ccccc2-n2c3cc(N(c4ccc5ccccc5c4)c4cccc5cc6c(cc45)C4(c5ccccc5-c5ccccc54)c4ccccc4-6)ccc3c3cc4c5ccccc5c5ccccc5c4cc32)cc1 ; c1ccc(-c2ccccc2-n2c3cc(N(c4ccc5ccccc5c4)c4ccc5cc6c(cc5c4)C4(c5ccccc5-c5ccccc54)c4ccccc4-6)ccc3c3cc4c5ccccc5c5ccccc5c4cc32)cc1]

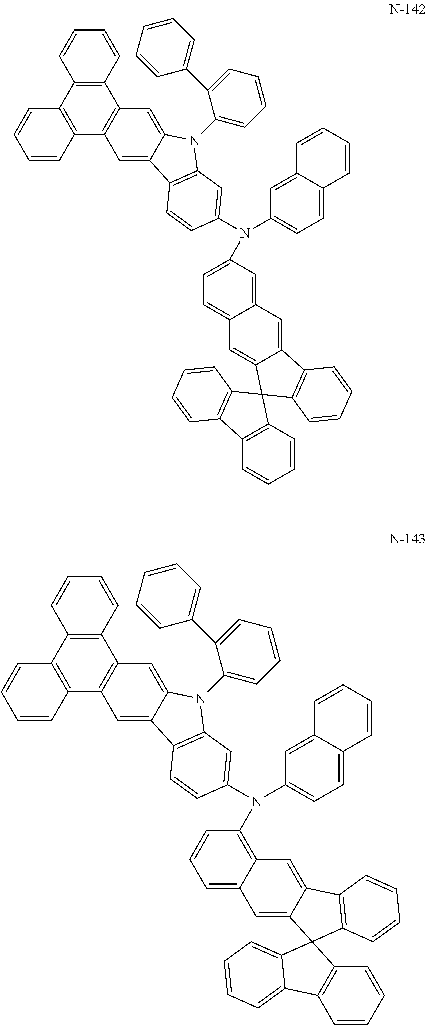 [SMILES: c1ccc(-c2ccccc2-n2c3cc(N(c4ccc5ccccc5c4)c4ccc5cc6c(cc5c4)-c4ccccc4C64c5ccccc5-c5ccccc54)ccc3c3cc4c5ccccc5c5ccccc5c4cc32)cc1 ; c1ccc(-c2ccccc2-n2c3cc(N(c4ccc5ccccc5c4)c4cccc5cc6c(cc45)-c4ccccc4C64c5ccccc5-c5ccccc54)ccc3c3cc4c5ccccc5c5ccccc5c4cc32)cc1]

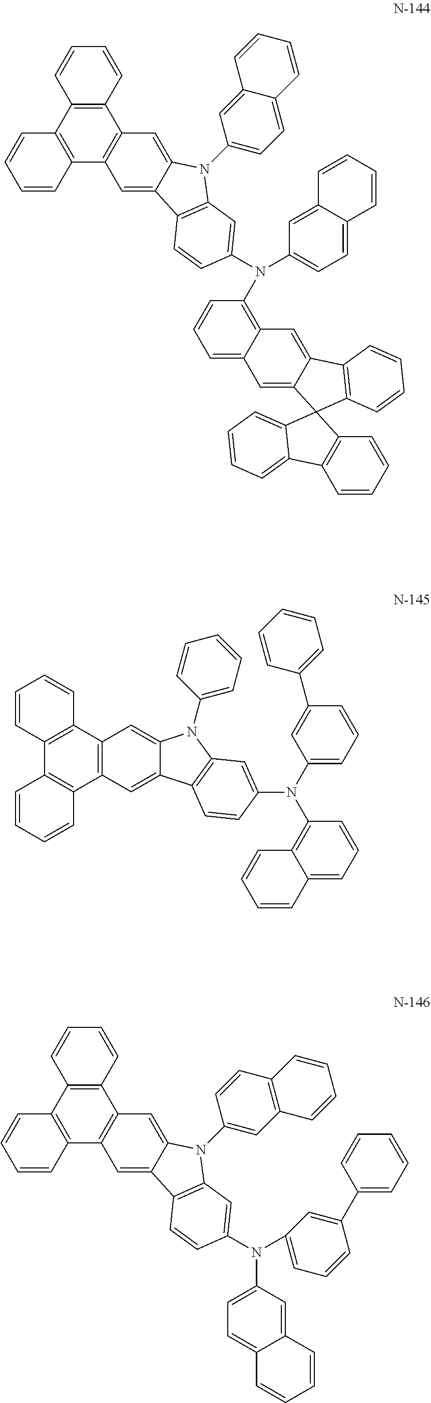 [SMILES: c1ccc2c(c1)-c1ccccc1C21c2ccccc2-c2cc3c(N(c4ccc5ccccc5c4)c4ccc5c6cc7c8ccccc8c8ccccc8c7cc6n(-c6ccc7ccccc7c6)c5c4)cccc3cc21 ; c1ccc(-c2cccc(N(c3ccc4c5cc6c7ccccc7c7ccccc7c6cc5n(-c5ccccc5)c4c3)c3cccc4ccccc34)c2)cc1 ; c1ccc(-c2cccc(N(c3ccc4ccccc4c3)c3ccc4c5cc6c7ccccc7c7ccccc7c6cc5n(-c5ccc6ccccc6c5)c4c3)c2)cc1]

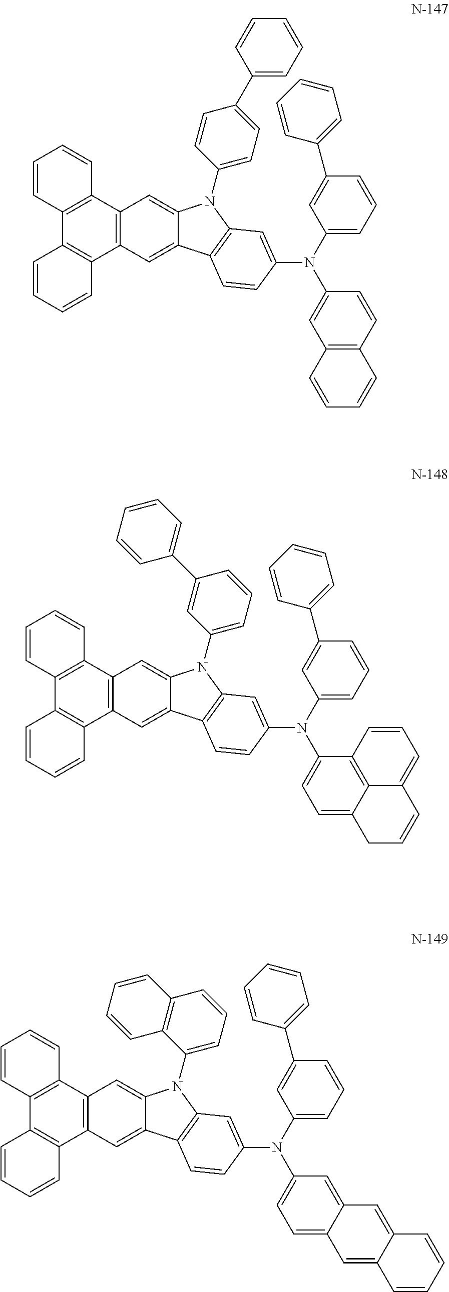 [SMILES: c1ccc(-c2ccc(-n3c4cc(N(c5cccc(-c6ccccc6)c5)c5ccc6ccccc6c5)ccc4c4cc5c6ccccc6c6ccccc6c5cc43)cc2)cc1 ; C1=Cc2cccc3c(N(c4cccc(-c5ccccc5)c4)c4ccc5c6cc7c8ccccc8c8ccccc8c7cc6n(-c6cccc(-c7ccccc7)c6)c5c4)ccc(c23)C1 ; c1ccc(-c2cccc(N(c3ccc4cc5ccccc5cc4c3)c3ccc4c5cc6c7ccccc7c7ccccc7c6cc5n(-c5cccc6ccccc56)c4c3)c2)cc1]

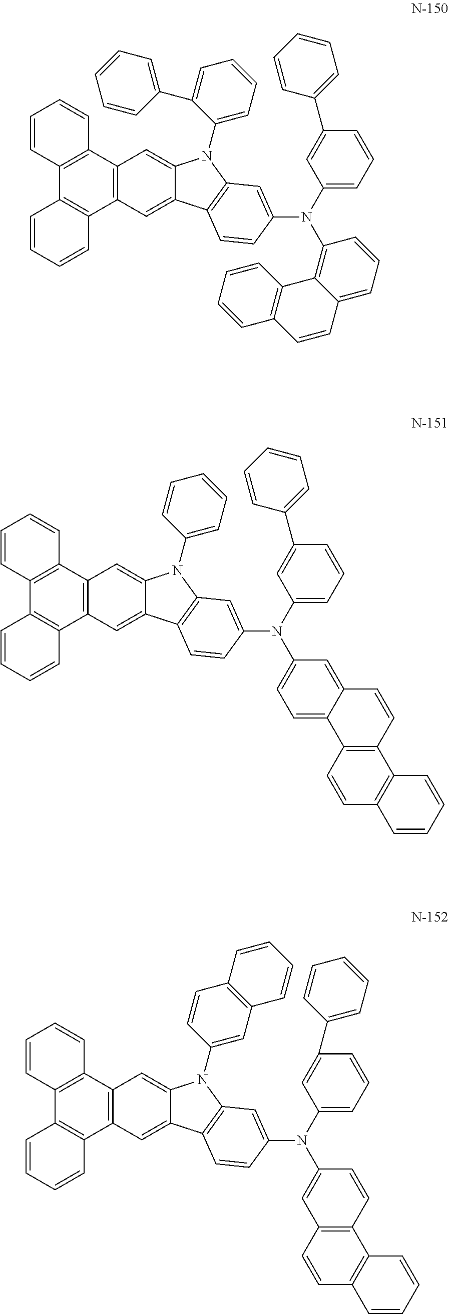 [SMILES: c1ccc(-c2cccc(N(c3ccc4c5cc6c7ccccc7c7ccccc7c6cc5n(-c5ccccc5-c5ccccc5)c4c3)c3cccc4ccc5ccccc5c34)c2)cc1 ; c1ccc(-c2cccc(N(c3ccc4c(ccc5c6ccccc6ccc45)c3)c3ccc4c5cc6c7ccccc7c7ccccc7c6cc5n(-c5ccccc5)c4c3)c2)cc1 ; c1ccc(-c2cccc(N(c3ccc4c(ccc5ccccc54)c3)c3ccc4c5cc6c7ccccc7c7ccccc7c6cc5n(-c5ccc6ccccc6c5)c4c3)c2)cc1]

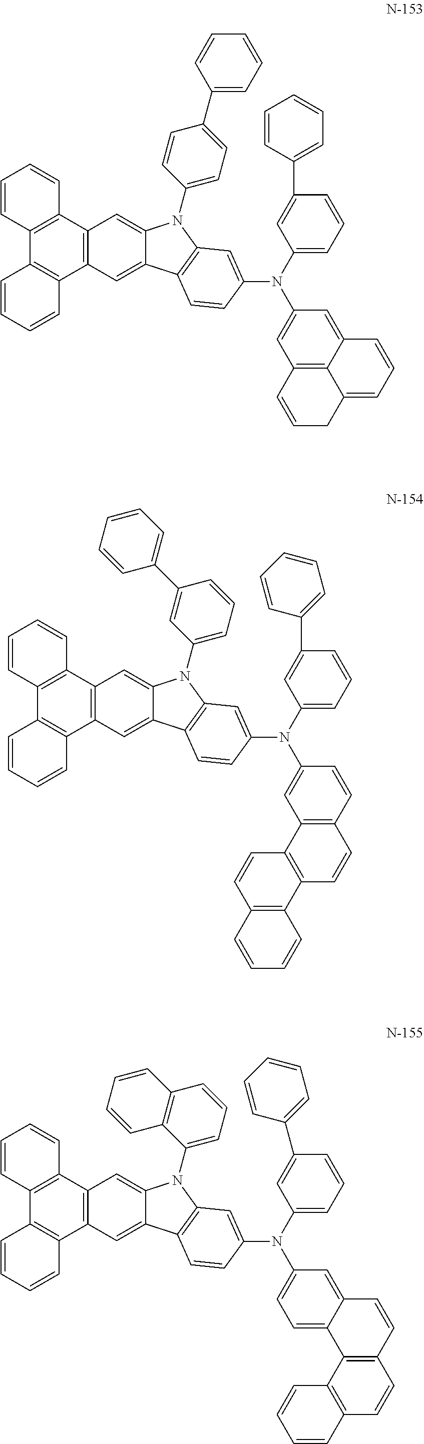 [SMILES: C1=Cc2cc(N(c3cccc(-c4ccccc4)c3)c3ccc4c5cc6c7ccccc7c7ccccc7c6cc5n(-c5ccc(-c6ccccc6)cc5)c4c3)cc3cccc(c23)C1 ; c1ccc(-c2cccc(N(c3ccc4ccc5c6ccccc6ccc5c4c3)c3ccc4c5cc6c7ccccc7c7ccccc7c6cc5n(-c5cccc(-c6ccccc6)c5)c4c3)c2)cc1 ; c1ccc(-c2cccc(N(c3ccc4c(ccc5ccc6ccccc6c54)c3)c3ccc4c5cc6c7ccccc7c7ccccc7c6cc5n(-c5cccc6ccccc56)c4c3)c2)cc1]

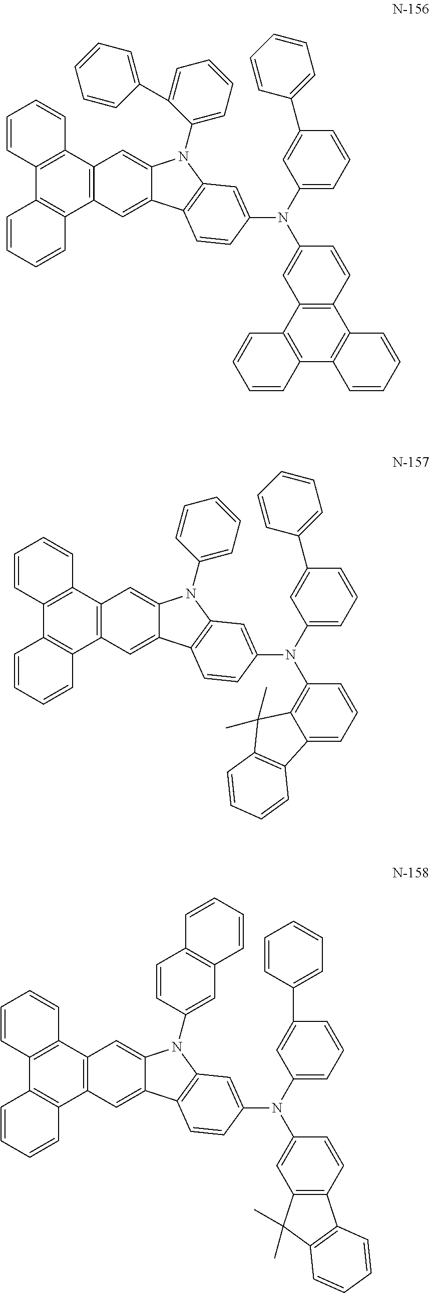 [SMILES: c1ccc(-c2cccc(N(c3ccc4c5ccccc5c5ccccc5c4c3)c3ccc4c5cc6c7ccccc7c7ccccc7c6cc5n(-c5ccccc5-c5ccccc5)c4c3)c2)cc1 ; CC1(C)c2ccccc2-c2cccc(N(c3cccc(-c4ccccc4)c3)c3ccc4c5cc6c7ccccc7c7ccccc7c6cc5n(-c5ccccc5)c4c3)c21 ; CC1(C)c2ccccc2-c2ccc(N(c3cccc(-c4ccccc4)c3)c3ccc4c5cc6c7ccccc7c7ccccc7c6cc5n(-c5ccc6ccccc6c5)c4c3)cc21]

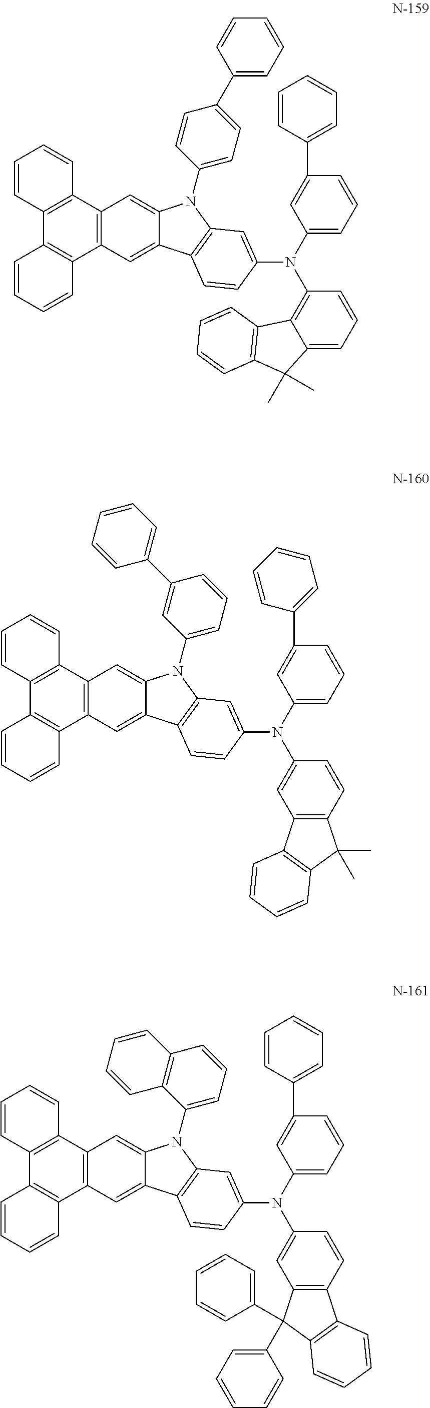 [SMILES: CC1(C)c2ccccc2-c2c(N(c3cccc(-c4ccccc4)c3)c3ccc4c5cc6c7ccccc7c7ccccc7c6cc5n(-c5ccc(-c6ccccc6)cc5)c4c3)cccc21 ; CC1(C)c2ccccc2-c2cc(N(c3cccc(-c4ccccc4)c3)c3ccc4c5cc6c7ccccc7c7ccccc7c6cc5n(-c5cccc(-c6ccccc6)c5)c4c3)ccc21 ; c1ccc(-c2cccc(N(c3ccc4c(c3)C(c3ccccc3)(c3ccccc3)c3ccccc3-4)c3ccc4c5cc6c7ccccc7c7ccccc7c6cc5n(-c5cccc6ccccc56)c4c3)c2)cc1]

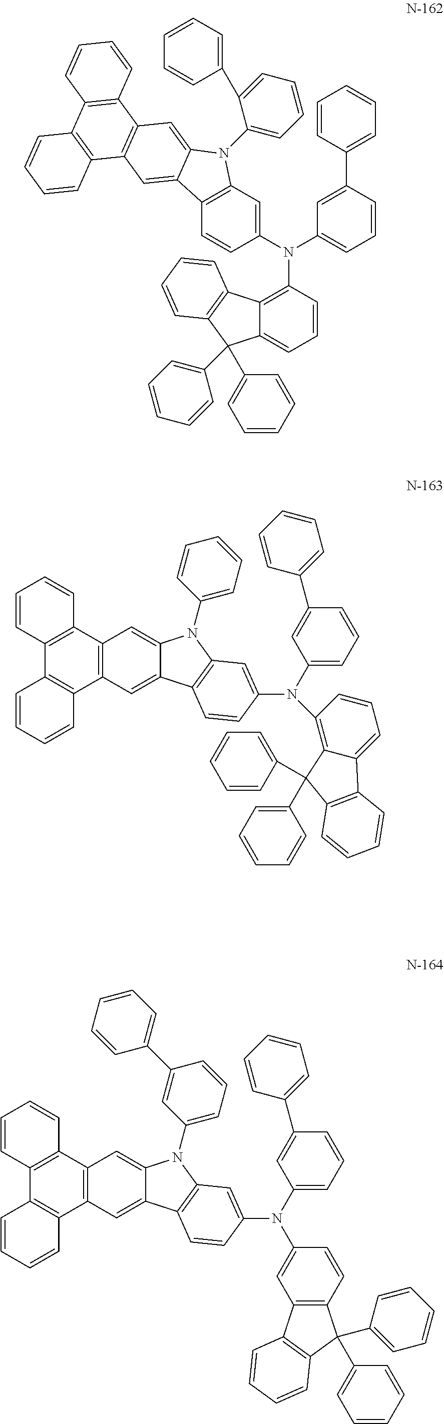 [SMILES: c1ccc(-c2cccc(N(c3ccc4c5cc6c7ccccc7c7ccccc7c6cc5n(-c5ccccc5-c5ccccc5)c4c3)c3cccc4c3-c3ccccc3C4(c3ccccc3)c3ccccc3)c2)cc1 ; c1ccc(-c2cccc(N(c3ccc4c5cc6c7ccccc7c7ccccc7c6cc5n(-c5ccccc5)c4c3)c3cccc4c3C(c3ccccc3)(c3ccccc3)c3ccccc3-4)c2)cc1 ; c1ccc(-c2cccc(N(c3ccc4c(c3)-c3ccccc3C4(c3ccccc3)c3ccccc3)c3ccc4c5cc6c7ccccc7c7ccccc7c6cc5n(-c5cccc(-c6ccccc6)c5)c4c3)c2)cc1]

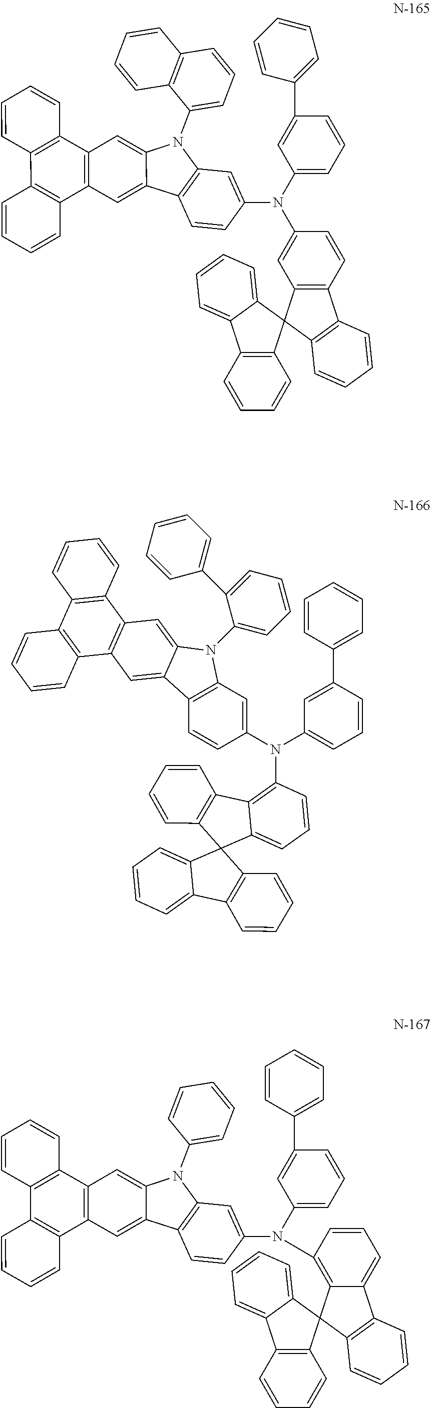 [SMILES: c1ccc(-c2cccc(N(c3ccc4c(c3)C3(c5ccccc5-c5ccccc53)c3ccccc3-4)c3ccc4c5cc6c7ccccc7c7ccccc7c6cc5n(-c5cccc6ccccc56)c4c3)c2)cc1 ; c1ccc(-c2cccc(N(c3ccc4c5cc6c7ccccc7c7ccccc7c6cc5n(-c5ccccc5-c5ccccc5)c4c3)c3cccc4c3-c3ccccc3C43c4ccccc4-c4ccccc43)c2)cc1 ; c1ccc(-c2cccc(N(c3ccc4c5cc6c7ccccc7c7ccccc7c6cc5n(-c5ccccc5)c4c3)c3cccc4c3C3(c5ccccc5-c5ccccc53)c3ccccc3-4)c2)cc1]

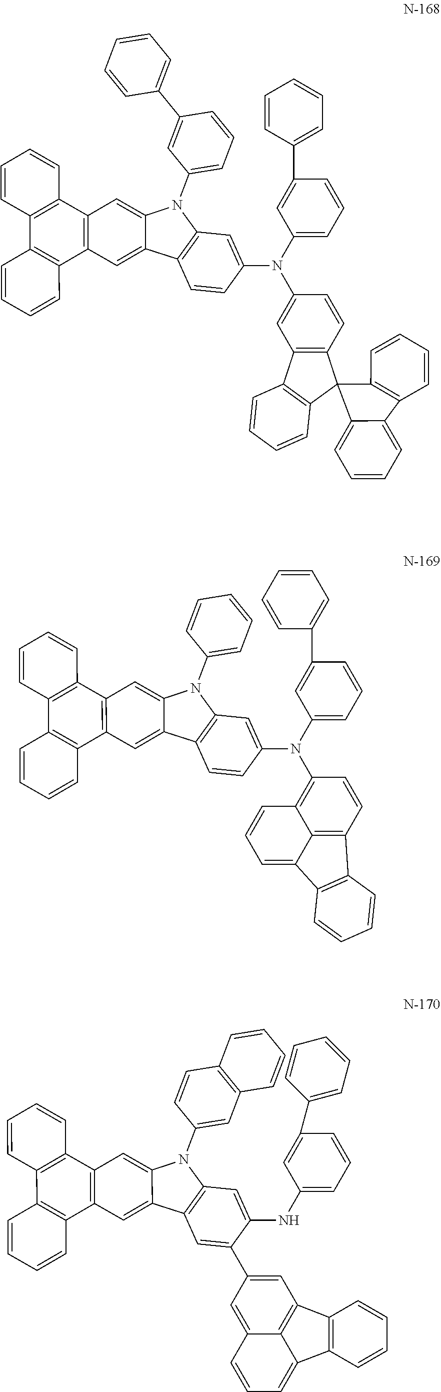 [SMILES: c1ccc(-c2cccc(N(c3ccc4c(c3)-c3ccccc3C43c4ccccc4-c4ccccc43)c3ccc4c5cc6c7ccccc7c7ccccc7c6cc5n(-c5cccc(-c6ccccc6)c5)c4c3)c2)cc1 ; c1ccc(-c2cccc(N(c3ccc4c5cc6c7ccccc7c7ccccc7c6cc5n(-c5ccccc5)c4c3)c3ccc4c5c(cccc35)-c3ccccc3-4)c2)cc1 ; c1ccc(-c2cccc(Nc3cc4c(cc3-c3cc5c6c(cccc6c3)-c3ccccc3-5)c3cc5c6ccccc6c6ccccc6c5cc3n4-c3ccc4ccccc4c3)c2)cc1]

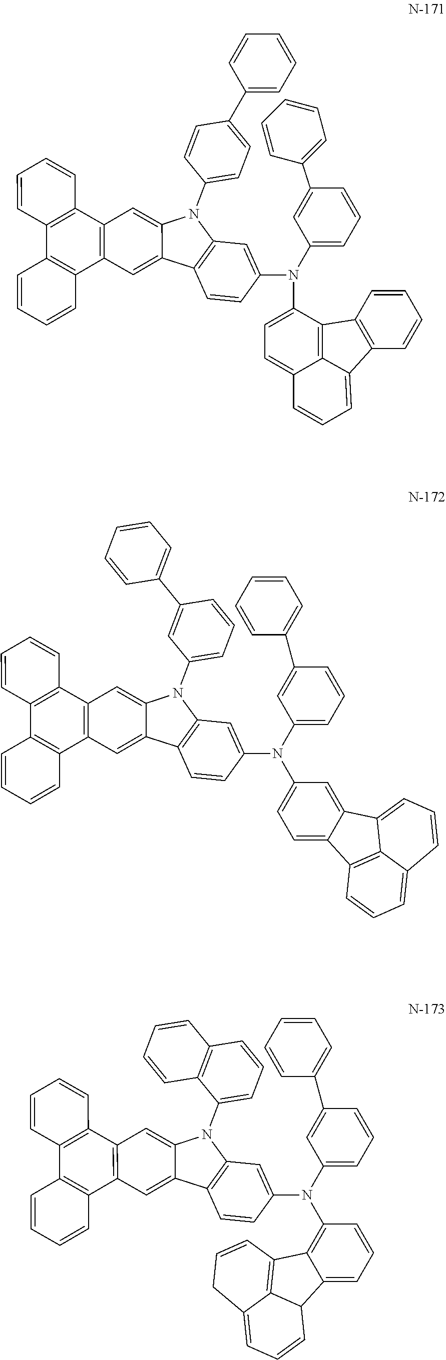 [SMILES: c1ccc(-c2ccc(-n3c4cc(N(c5cccc(-c6ccccc6)c5)c5ccc6cccc7c6c5-c5ccccc5-7)ccc4c4cc5c6ccccc6c6ccccc6c5cc43)cc2)cc1 ; c1ccc(-c2cccc(N(c3ccc4c(c3)-c3cccc5cccc-4c35)c3ccc4c5cc6c7ccccc7c7ccccc7c6cc5n(-c5cccc(-c6ccccc6)c5)c4c3)c2)cc1 ; C1=CC2C3=C(C=CCC3=C1)c1c2cccc1N(c1cccc(-c2ccccc2)c1)c1ccc2c3cc4c5ccccc5c5ccccc5c4cc3n(-c3cccc4ccccc34)c2c1]

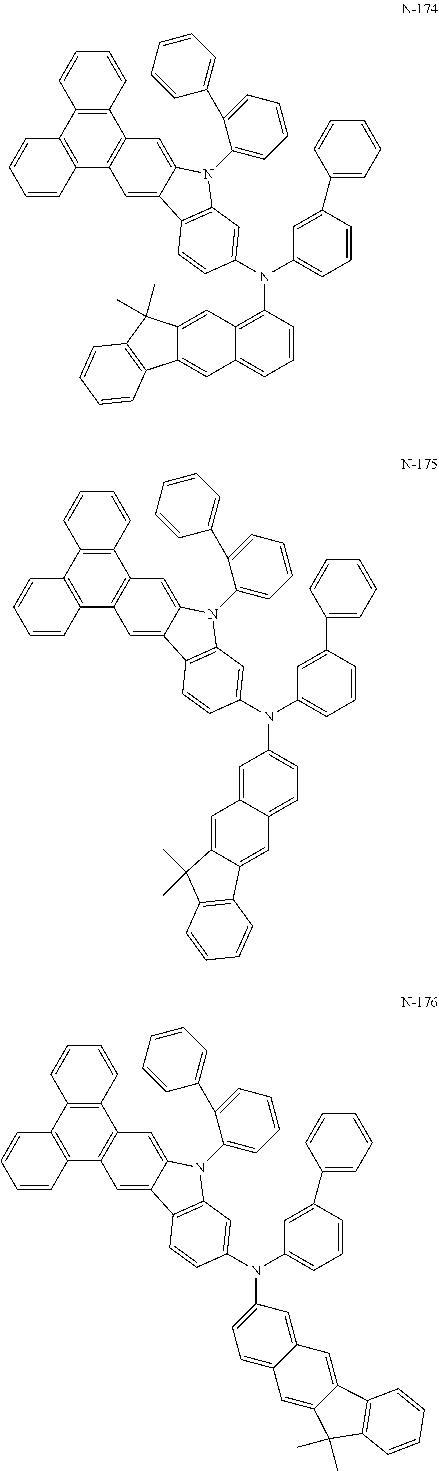 [SMILES: CC1(C)c2ccccc2-c2cc3cccc(N(c4cccc(-c5ccccc5)c4)c4ccc5c6cc7c8ccccc8c8ccccc8c7cc6n(-c6ccccc6-c6ccccc6)c5c4)c3cc21 ; CC1(C)c2ccccc2-c2cc3ccc(N(c4cccc(-c5ccccc5)c4)c4ccc5c6cc7c8ccccc8c8ccccc8c7cc6n(-c6ccccc6-c6ccccc6)c5c4)cc3cc21 ; CC1(C)c2ccccc2-c2cc3cc(N(c4cccc(-c5ccccc5)c4)c4ccc5c6cc7c8ccccc8c8ccccc8c7cc6n(-c6ccccc6-c6ccccc6)c5c4)ccc3cc21]

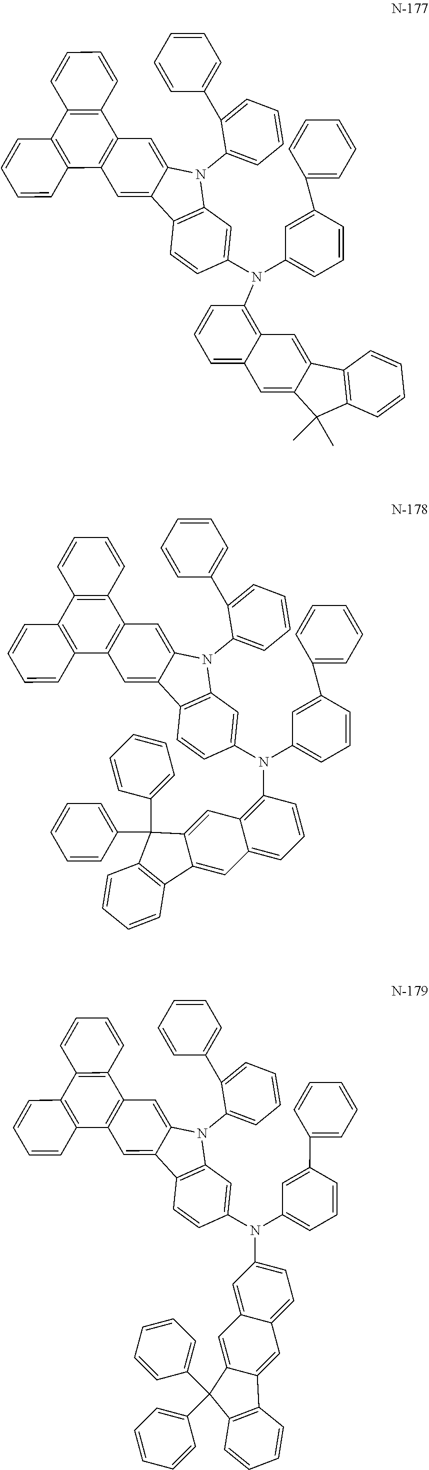 [SMILES: CC1(C)c2ccccc2-c2cc3c(N(c4cccc(-c5ccccc5)c4)c4ccc5c6cc7c8ccccc8c8ccccc8c7cc6n(-c6ccccc6-c6ccccc6)c5c4)cccc3cc21 ; c1ccc(-c2cccc(N(c3ccc4c5cc6c7ccccc7c7ccccc7c6cc5n(-c5ccccc5-c5ccccc5)c4c3)c3cccc4cc5c(cc34)C(c3ccccc3)(c3ccccc3)c3ccccc3-5)c2)cc1 ; c1ccc(-c2cccc(N(c3ccc4cc5c(cc4c3)C(c3ccccc3)(c3ccccc3)c3ccccc3-5)c3ccc4c5cc6c7ccccc7c7ccccc7c6cc5n(-c5ccccc5-c5ccccc5)c4c3)c2)cc1]

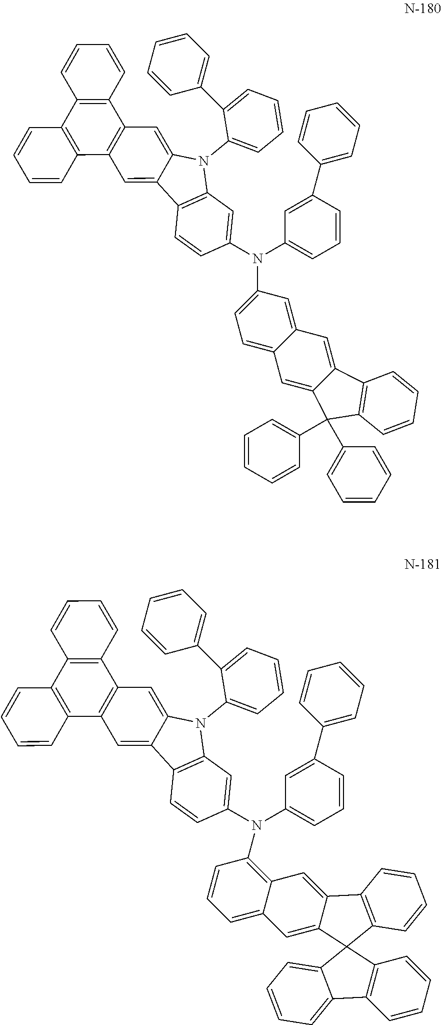 [SMILES: c1ccc(-c2cccc(N(c3ccc4cc5c(cc4c3)-c3ccccc3C5(c3ccccc3)c3ccccc3)c3ccc4c5cc6c7ccccc7c7ccccc7c6cc5n(-c5ccccc5-c5ccccc5)c4c3)c2)cc1 ; c1ccc(-c2cccc(N(c3ccc4c5cc6c7ccccc7c7ccccc7c6cc5n(-c5ccccc5-c5ccccc5)c4c3)c3cccc4cc5c(cc34)-c3ccccc3C53c4ccccc4-c4ccccc43)c2)cc1]

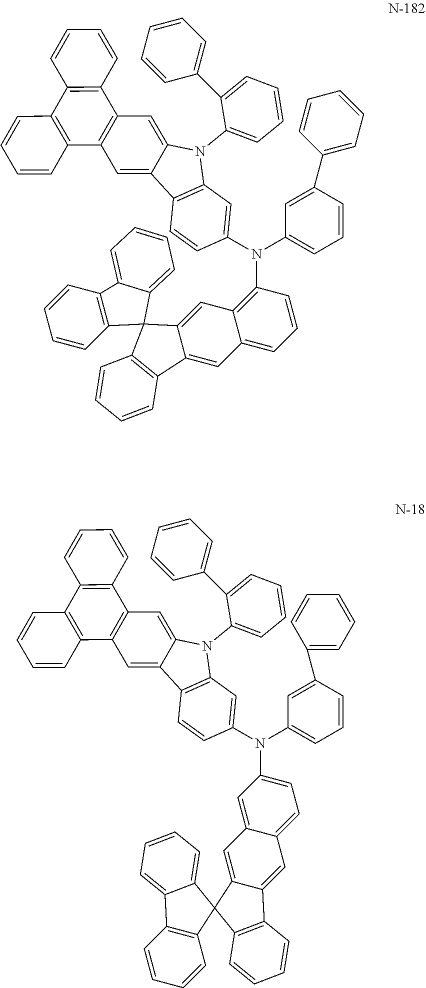 [SMILES: c1ccc(-c2cccc(N(c3ccc4c5cc6c7ccccc7c7ccccc7c6cc5n(-c5ccccc5-c5ccccc5)c4c3)c3cccc4cc5c(cc34)C3(c4ccccc4-c4ccccc43)c3ccccc3-5)c2)cc1 ; c1ccc(-c2cccc(N(c3ccc4cc5c(cc4c3)C3(c4ccccc4-c4ccccc43)c3ccccc3-5)c3ccc4c5cc6c7ccccc7c7ccccc7c6cc5n(-c5ccccc5-c5ccccc5)c4c3)c2)cc1]

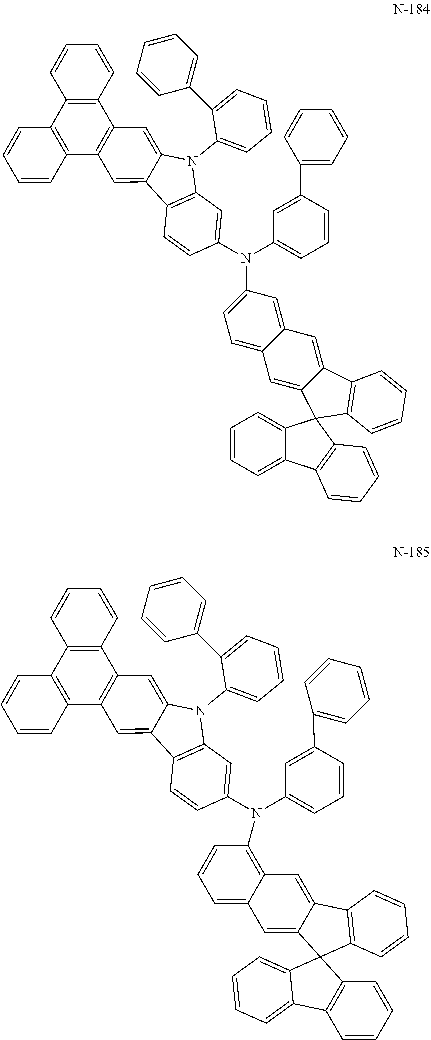 [SMILES: c1ccc(-c2cccc(N(c3ccc4cc5c(cc4c3)-c3ccccc3C53c4ccccc4-c4ccccc43)c3ccc4c5cc6c7ccccc7c7ccccc7c6cc5n(-c5ccccc5-c5ccccc5)c4c3)c2)cc1 ; c1ccc(-c2cccc(N(c3ccc4c5cc6c7ccccc7c7ccccc7c6cc5n(-c5ccccc5-c5ccccc5)c4c3)c3cccc4cc5c(cc34)-c3ccccc3C53c4ccccc4-c4ccccc43)c2)cc1]

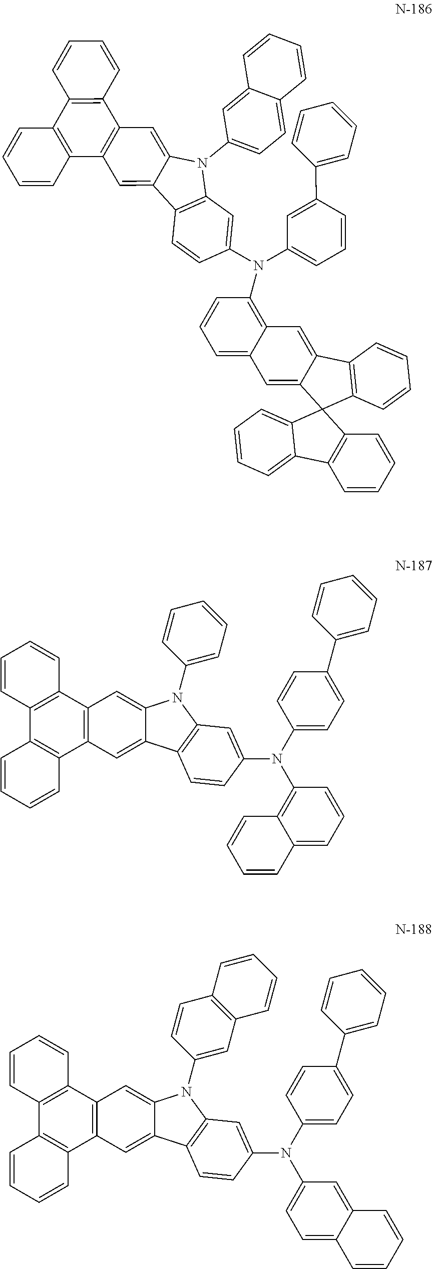 [SMILES: c1ccc(-c2cccc(N(c3ccc4c5cc6c7ccccc7c7ccccc7c6cc5n(-c5ccc6ccccc6c5)c4c3)c3cccc4cc5c(cc34)-c3ccccc3C53c4ccccc4-c4ccccc43)c2)cc1 ; c1ccc(-c2ccc(N(c3ccc4c5cc6c7ccccc7c7ccccc7c6cc5n(-c5ccccc5)c4c3)c3cccc4ccccc34)cc2)cc1 ; c1ccc(-c2ccc(N(c3ccc4ccccc4c3)c3ccc4c5cc6c7ccccc7c7ccccc7c6cc5n(-c5ccc6ccccc6c5)c4c3)cc2)cc1]

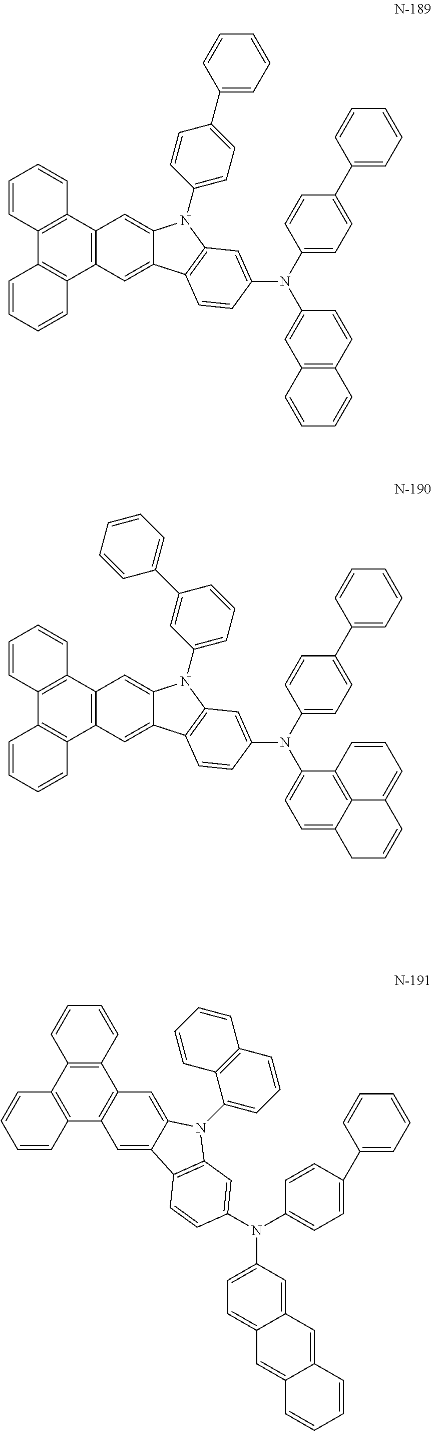 [SMILES: c1ccc(-c2ccc(N(c3ccc4ccccc4c3)c3ccc4c5cc6c7ccccc7c7ccccc7c6cc5n(-c5ccc(-c6ccccc6)cc5)c4c3)cc2)cc1 ; C1=Cc2cccc3c(N(c4ccc(-c5ccccc5)cc4)c4ccc5c6cc7c8ccccc8c8ccccc8c7cc6n(-c6cccc(-c7ccccc7)c6)c5c4)ccc(c23)C1 ; c1ccc(-c2ccc(N(c3ccc4cc5ccccc5cc4c3)c3ccc4c5cc6c7ccccc7c7ccccc7c6cc5n(-c5cccc6ccccc56)c4c3)cc2)cc1]

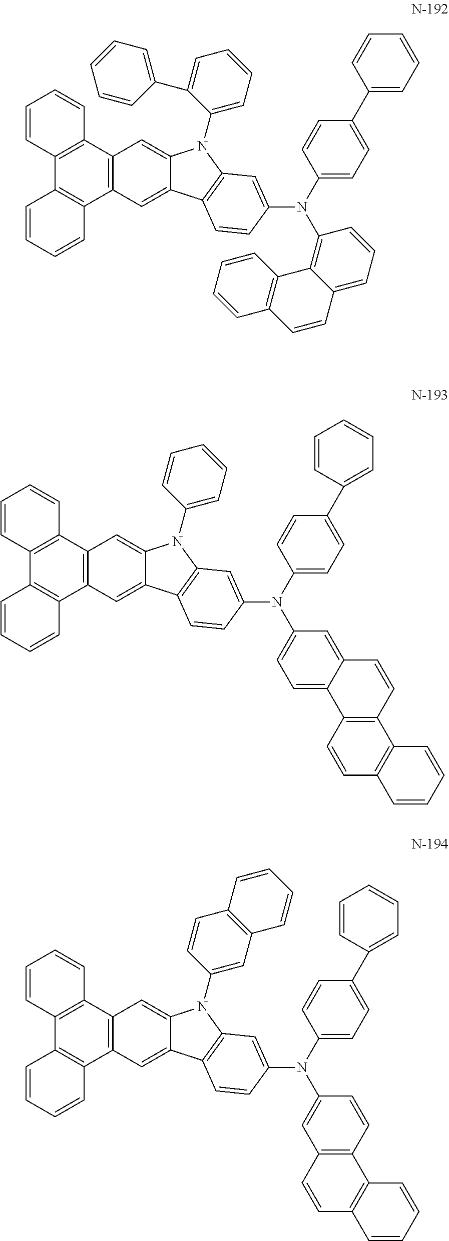 [SMILES: c1ccc(-c2ccc(N(c3ccc4c5cc6c7ccccc7c7ccccc7c6cc5n(-c5ccccc5-c5ccccc5)c4c3)c3cccc4ccc5ccccc5c34)cc2)cc1 ; c1ccc(-c2ccc(N(c3ccc4c(ccc5c6ccccc6ccc45)c3)c3ccc4c5cc6c7ccccc7c7ccccc7c6cc5n(-c5ccccc5)c4c3)cc2)cc1 ; c1ccc(-c2ccc(N(c3ccc4c(ccc5ccccc54)c3)c3ccc4c5cc6c7ccccc7c7ccccc7c6cc5n(-c5ccc6ccccc6c5)c4c3)cc2)cc1]

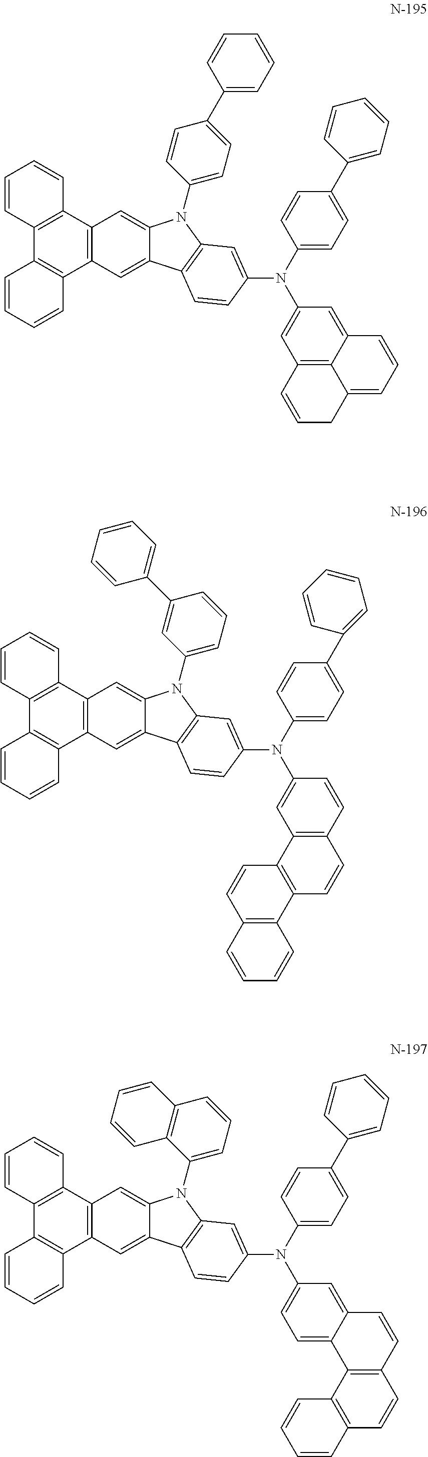 [SMILES: C1=Cc2cc(N(c3ccc(-c4ccccc4)cc3)c3ccc4c5cc6c7ccccc7c7ccccc7c6cc5n(-c5ccc(-c6ccccc6)cc5)c4c3)cc3cccc(c23)C1 ; c1ccc(-c2ccc(N(c3ccc4ccc5c6ccccc6ccc5c4c3)c3ccc4c5cc6c7ccccc7c7ccccc7c6cc5n(-c5cccc(-c6ccccc6)c5)c4c3)cc2)cc1 ; c1ccc(-c2ccc(N(c3ccc4c(ccc5ccc6ccccc6c54)c3)c3ccc4c5cc6c7ccccc7c7ccccc7c6cc5n(-c5cccc6ccccc56)c4c3)cc2)cc1]

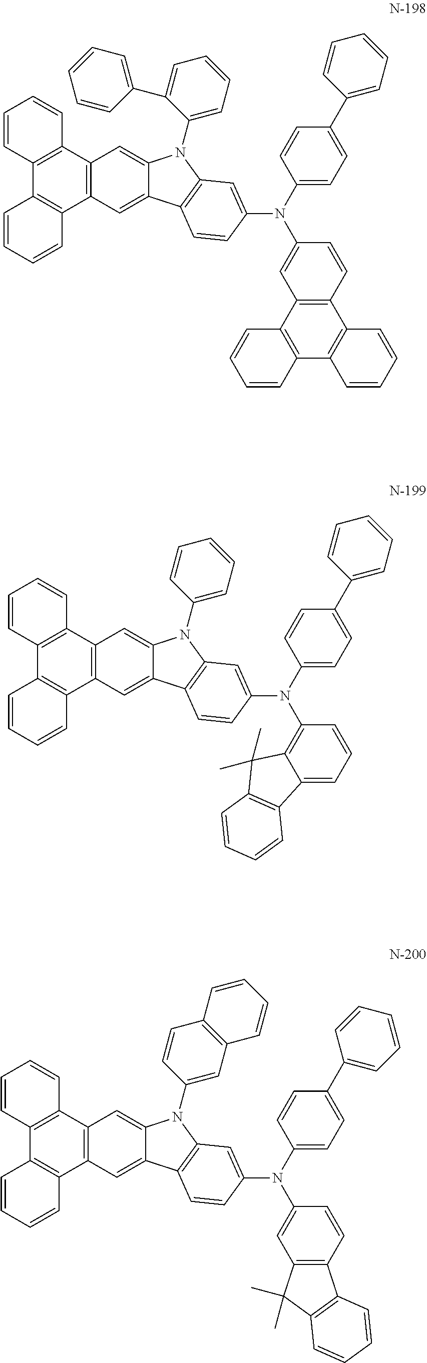 [SMILES: c1ccc(-c2ccc(N(c3ccc4c5ccccc5c5ccccc5c4c3)c3ccc4c5cc6c7ccccc7c7ccccc7c6cc5n(-c5ccccc5-c5ccccc5)c4c3)cc2)cc1 ; CC1(C)c2ccccc2-c2cccc(N(c3ccc(-c4ccccc4)cc3)c3ccc4c5cc6c7ccccc7c7ccccc7c6cc5n(-c5ccccc5)c4c3)c21 ; CC1(C)c2ccccc2-c2ccc(N(c3ccc(-c4ccccc4)cc3)c3ccc4c5cc6c7ccccc7c7ccccc7c6cc5n(-c5ccc6ccccc6c5)c4c3)cc21]

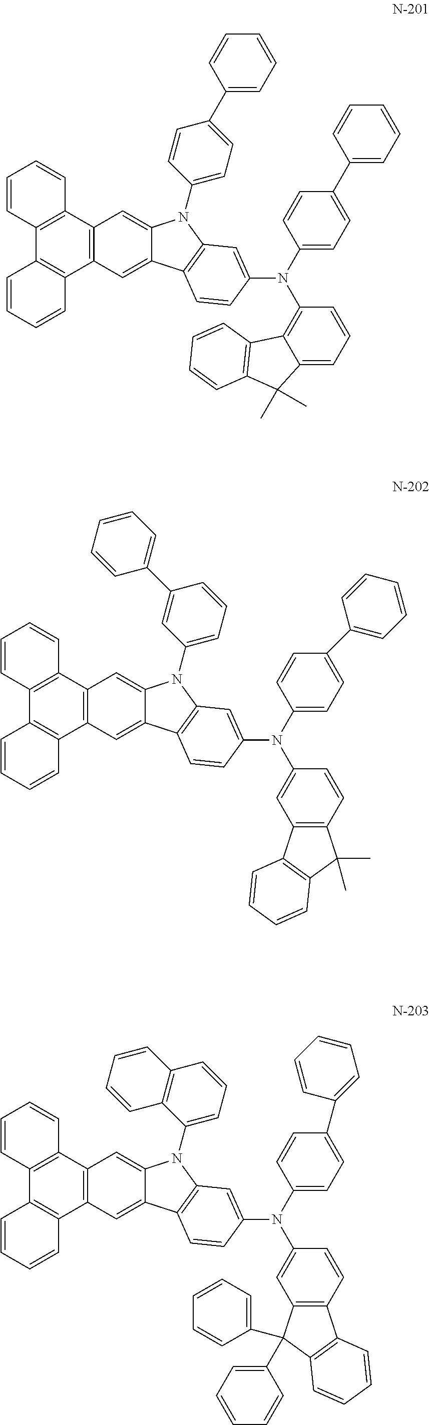 [SMILES: CC1(C)c2ccccc2-c2c(N(c3ccc(-c4ccccc4)cc3)c3ccc4c5cc6c7ccccc7c7ccccc7c6cc5n(-c5ccc(-c6ccccc6)cc5)c4c3)cccc21 ; CC1(C)c2ccccc2-c2cc(N(c3ccc(-c4ccccc4)cc3)c3ccc4c5cc6c7ccccc7c7ccccc7c6cc5n(-c5cccc(-c6ccccc6)c5)c4c3)ccc21 ; c1ccc(-c2ccc(N(c3ccc4c(c3)C(c3ccccc3)(c3ccccc3)c3ccccc3-4)c3ccc4c5cc6c7ccccc7c7ccccc7c6cc5n(-c5cccc6ccccc56)c4c3)cc2)cc1]

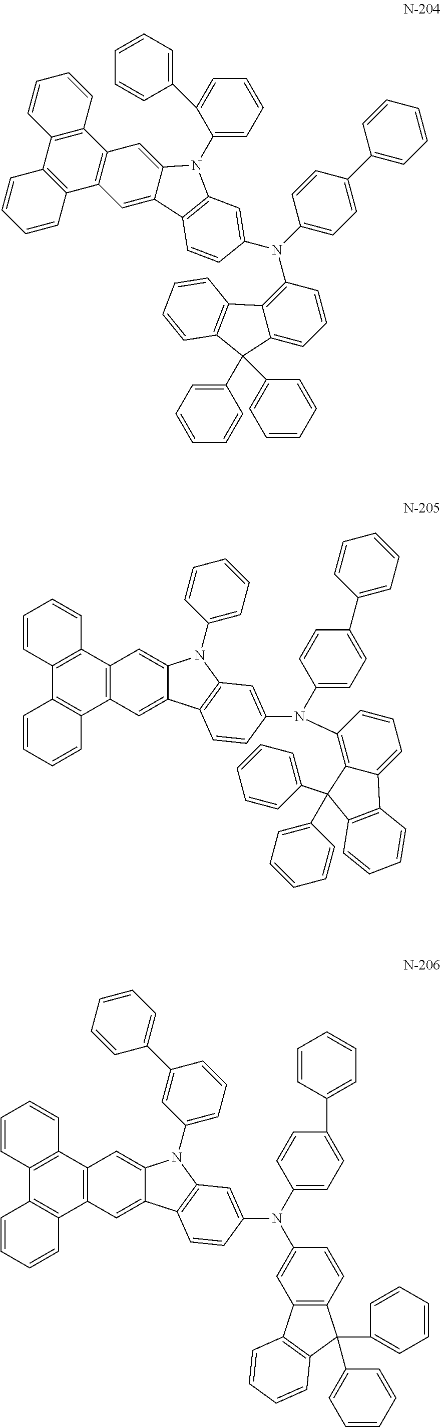 [SMILES: c1ccc(-c2ccc(N(c3ccc4c5cc6c7ccccc7c7ccccc7c6cc5n(-c5ccccc5-c5ccccc5)c4c3)c3cccc4c3-c3ccccc3C4(c3ccccc3)c3ccccc3)cc2)cc1 ; c1ccc(-c2ccc(N(c3ccc4c5cc6c7ccccc7c7ccccc7c6cc5n(-c5ccccc5)c4c3)c3cccc4c3C(c3ccccc3)(c3ccccc3)c3ccccc3-4)cc2)cc1 ; c1ccc(-c2ccc(N(c3ccc4c(c3)-c3ccccc3C4(c3ccccc3)c3ccccc3)c3ccc4c5cc6c7ccccc7c7ccccc7c6cc5n(-c5cccc(-c6ccccc6)c5)c4c3)cc2)cc1]

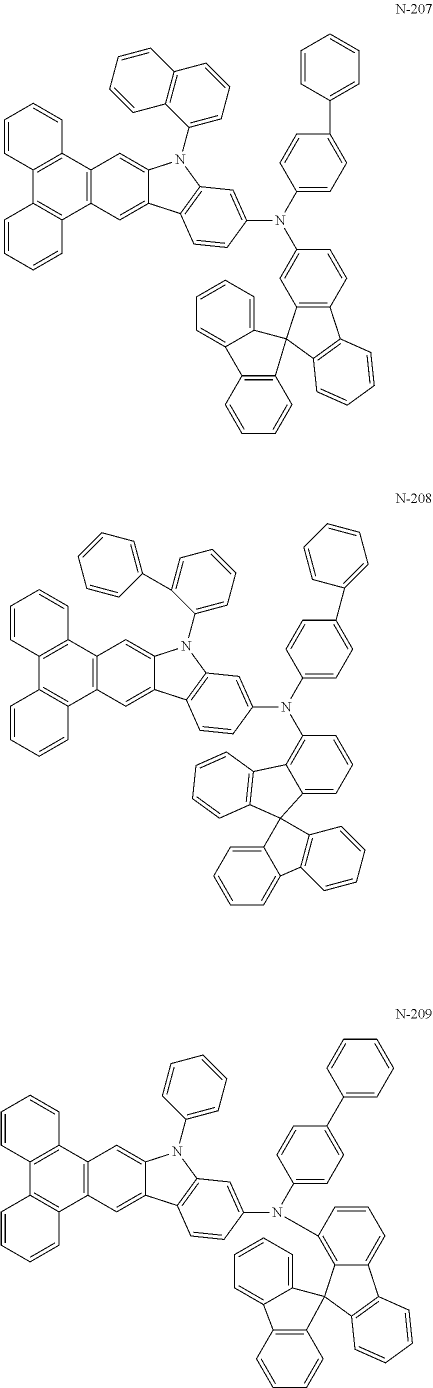 [SMILES: c1ccc(-c2ccc(N(c3ccc4c(c3)C3(c5ccccc5-c5ccccc53)c3ccccc3-4)c3ccc4c5cc6c7ccccc7c7ccccc7c6cc5n(-c5cccc6ccccc56)c4c3)cc2)cc1 ; c1ccc(-c2ccc(N(c3ccc4c5cc6c7ccccc7c7ccccc7c6cc5n(-c5ccccc5-c5ccccc5)c4c3)c3cccc4c3-c3ccccc3C43c4ccccc4-c4ccccc43)cc2)cc1 ; c1ccc(-c2ccc(N(c3ccc4c5cc6c7ccccc7c7ccccc7c6cc5n(-c5ccccc5)c4c3)c3cccc4c3C3(c5ccccc5-c5ccccc53)c3ccccc3-4)cc2)cc1]

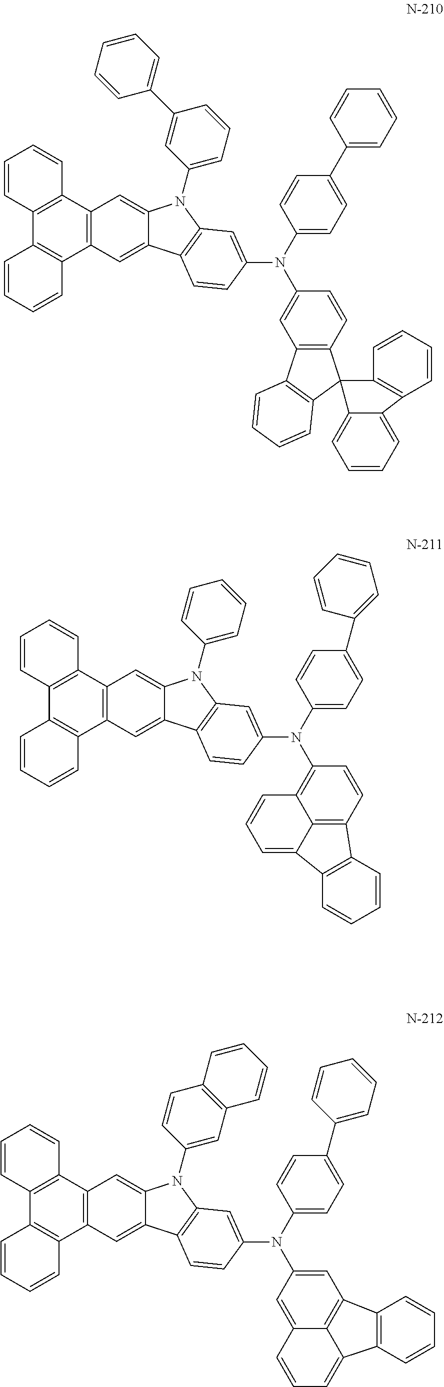 [SMILES: c1ccc(-c2ccc(N(c3ccc4c(c3)-c3ccccc3C43c4ccccc4-c4ccccc43)c3ccc4c5cc6c7ccccc7c7ccccc7c6cc5n(-c5cccc(-c6ccccc6)c5)c4c3)cc2)cc1 ; c1ccc(-c2ccc(N(c3ccc4c5cc6c7ccccc7c7ccccc7c6cc5n(-c5ccccc5)c4c3)c3ccc4c5c(cccc35)-c3ccccc3-4)cc2)cc1 ; c1ccc(-c2ccc(N(c3cc4c5c(cccc5c3)-c3ccccc3-4)c3ccc4c5cc6c7ccccc7c7ccccc7c6cc5n(-c5ccc6ccccc6c5)c4c3)cc2)cc1]

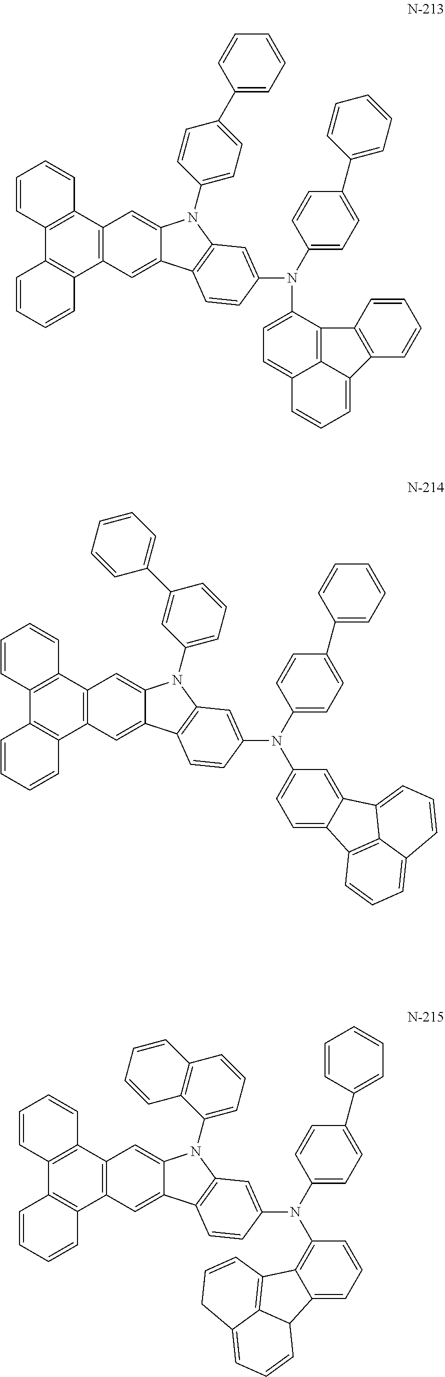 [SMILES: c1ccc(-c2ccc(N(c3ccc4c5cc6c7ccccc7c7ccccc7c6cc5n(-c5ccc(-c6ccccc6)cc5)c4c3)c3ccc4cccc5c4c3-c3ccccc3-5)cc2)cc1 ; c1ccc(-c2ccc(N(c3ccc4c(c3)-c3cccc5cccc-4c35)c3ccc4c5cc6c7ccccc7c7ccccc7c6cc5n(-c5cccc(-c6ccccc6)c5)c4c3)cc2)cc1 ; C1=CC2C3=C(C=CCC3=C1)c1c2cccc1N(c1ccc(-c2ccccc2)cc1)c1ccc2c3cc4c5ccccc5c5ccccc5c4cc3n(-c3cccc4ccccc34)c2c1]

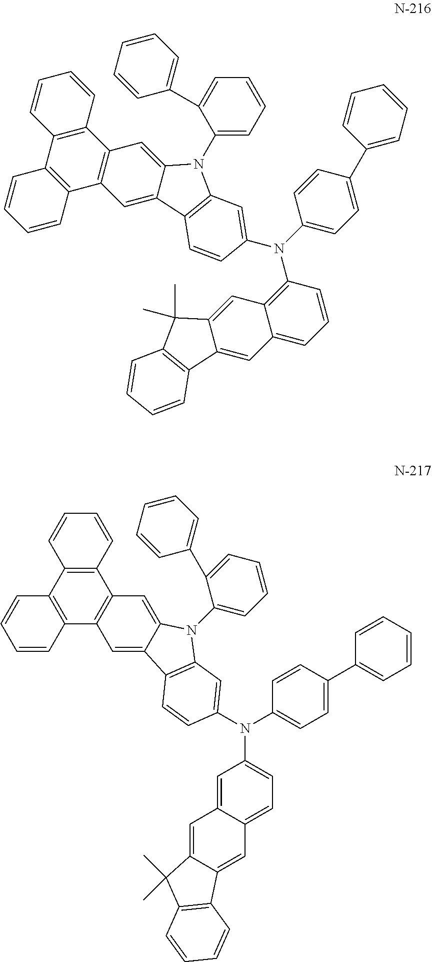 [SMILES: CC1(C)c2ccccc2-c2cc3cccc(N(c4ccc(-c5ccccc5)cc4)c4ccc5c6cc7c8ccccc8c8ccccc8c7cc6n(-c6ccccc6-c6ccccc6)c5c4)c3cc21 ; CC1(C)c2ccccc2-c2cc3ccc(N(c4ccc(-c5ccccc5)cc4)c4ccc5c6cc7c8ccccc8c8ccccc8c7cc6n(-c6ccccc6-c6ccccc6)c5c4)cc3cc21]

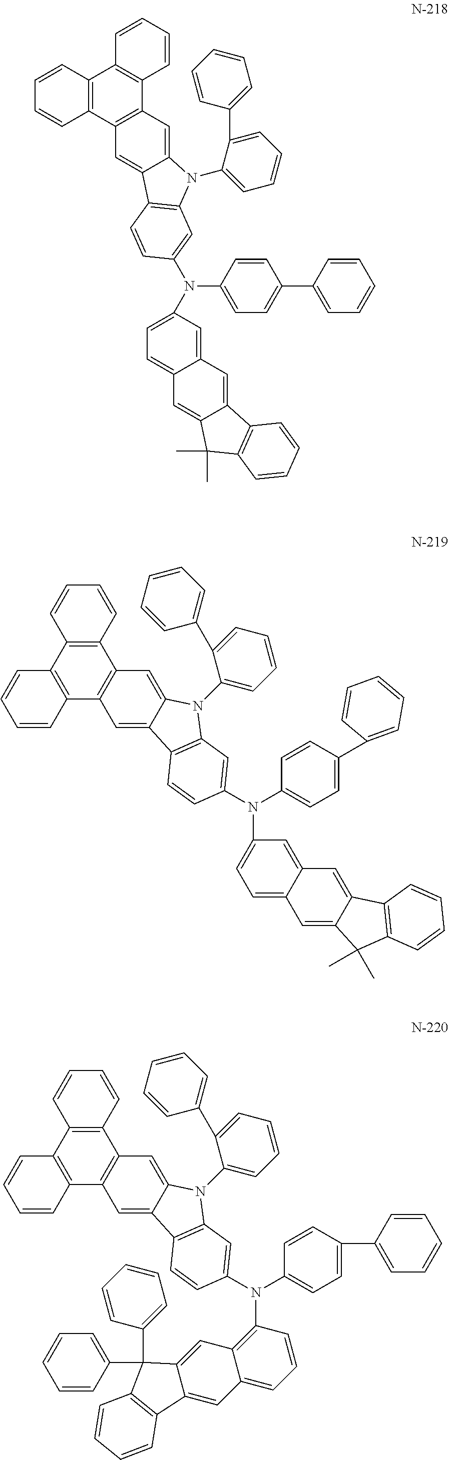 [SMILES: CC1(C)c2ccccc2-c2cc3cc(N(c4ccc(-c5ccccc5)cc4)c4ccc5c6cc7c8ccccc8c8ccccc8c7cc6n(-c6ccccc6-c6ccccc6)c5c4)ccc3cc21 ; CC1(C)c2ccccc2-c2cc3cc(N(c4ccc(-c5ccccc5)cc4)c4ccc5c6cc7c8ccccc8c8ccccc8c7cc6n(-c6ccccc6-c6ccccc6)c5c4)ccc3cc21 ; c1ccc(-c2ccc(N(c3ccc4c5cc6c7ccccc7c7ccccc7c6cc5n(-c5ccccc5-c5ccccc5)c4c3)c3cccc4cc5c(cc34)C(c3ccccc3)(c3ccccc3)c3ccccc3-5)cc2)cc1]

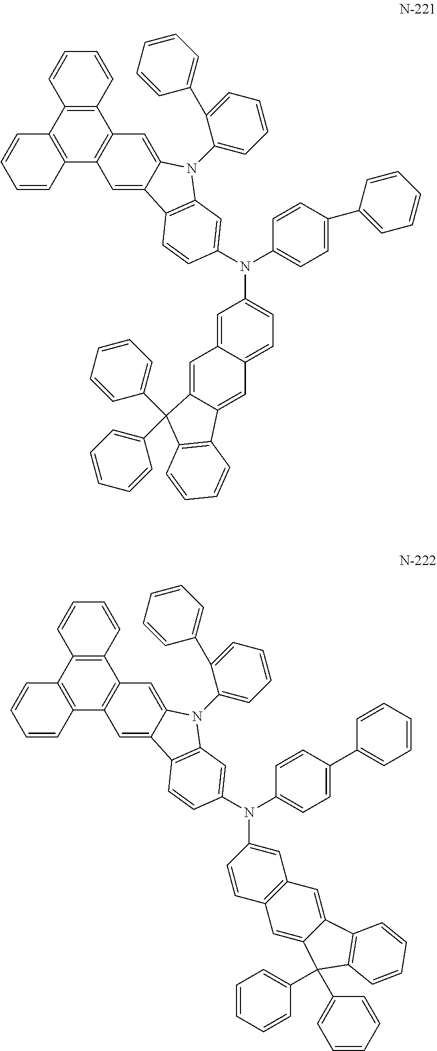 [SMILES: c1ccc(-c2ccc(N(c3ccc4cc5c(cc4c3)C(c3ccccc3)(c3ccccc3)c3ccccc3-5)c3ccc4c5cc6c7ccccc7c7ccccc7c6cc5n(-c5ccccc5-c5ccccc5)c4c3)cc2)cc1 ; c1ccc(-c2ccc(N(c3ccc4cc5c(cc4c3)-c3ccccc3C5(c3ccccc3)c3ccccc3)c3ccc4c5cc6c7ccccc7c7ccccc7c6cc5n(-c5ccccc5-c5ccccc5)c4c3)cc2)cc1]

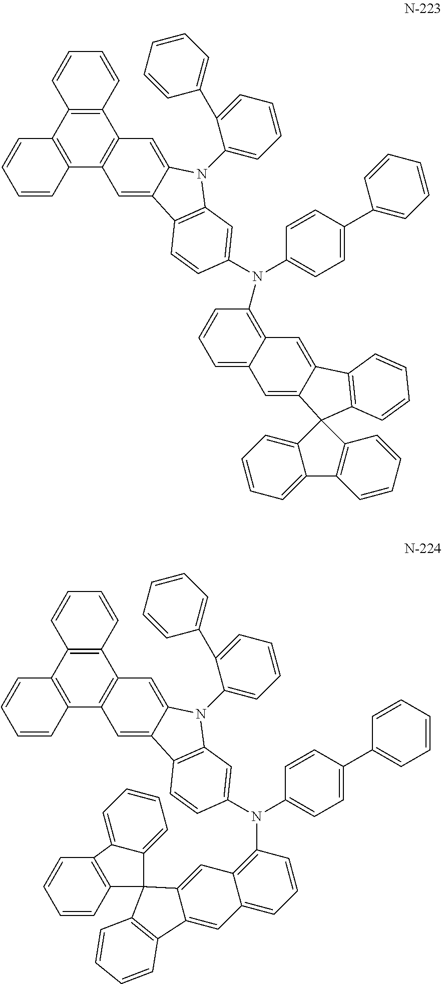 [SMILES: c1ccc(-c2ccc(N(c3ccc4c5cc6c7ccccc7c7ccccc7c6cc5n(-c5ccccc5-c5ccccc5)c4c3)c3cccc4cc5c(cc34)-c3ccccc3C53c4ccccc4-c4ccccc43)cc2)cc1 ; c1ccc(-c2ccc(N(c3ccc4c5cc6c7ccccc7c7ccccc7c6cc5n(-c5ccccc5-c5ccccc5)c4c3)c3cccc4cc5c(cc34)C3(c4ccccc4-c4ccccc43)c3ccccc3-5)cc2)cc1]

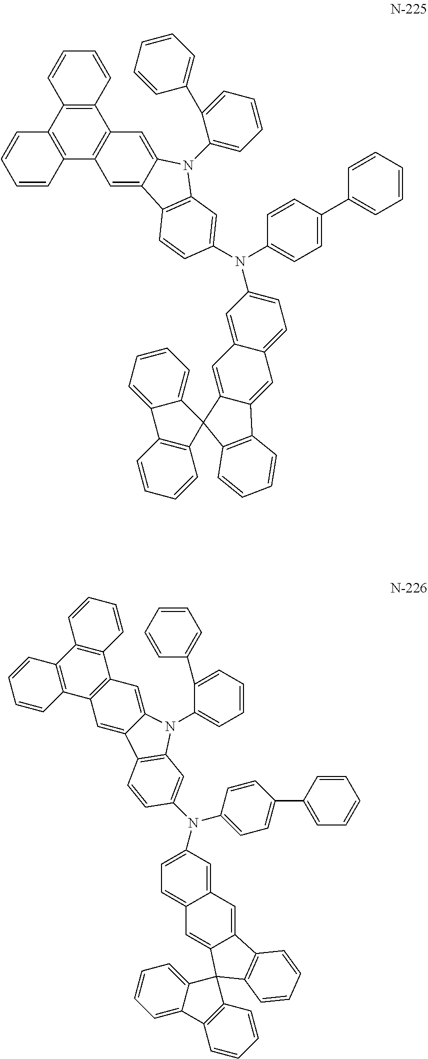 [SMILES: c1ccc(-c2ccc(N(c3ccc4cc5c(cc4c3)C3(c4ccccc4-c4ccccc43)c3ccccc3-5)c3ccc4c5cc6c7ccccc7c7ccccc7c6cc5n(-c5ccccc5-c5ccccc5)c4c3)cc2)cc1 ; c1ccc(-c2ccc(N(c3ccc4cc5c(cc4c3)-c3ccccc3C53c4ccccc4-c4ccccc43)c3ccc4c5cc6c7ccccc7c7ccccc7c6cc5n(-c5ccccc5-c5ccccc5)c4c3)cc2)cc1]

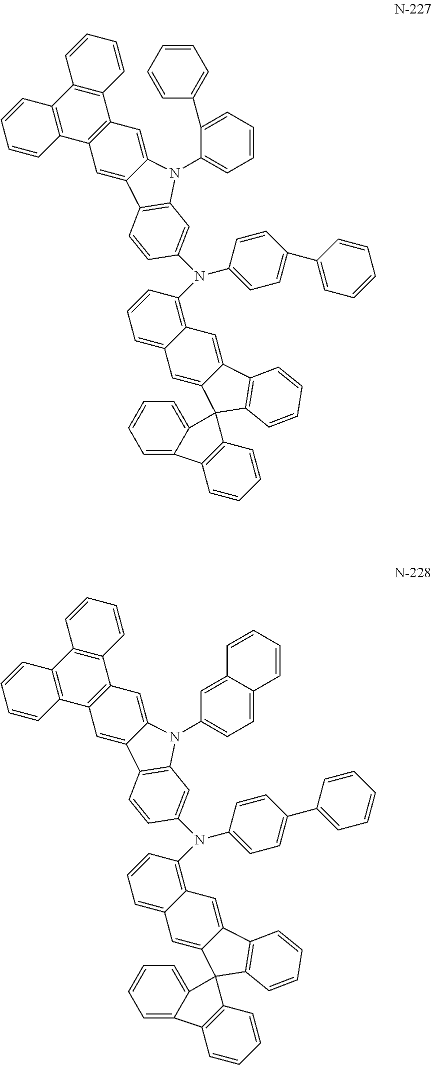 [SMILES: c1ccc(-c2ccc(N(c3ccc4c5cc6c7ccccc7c7ccccc7c6cc5n(-c5ccccc5-c5ccccc5)c4c3)c3cccc4cc5c(cc34)-c3ccccc3C53c4ccccc4-c4ccccc43)cc2)cc1 ; c1ccc(-c2ccc(N(c3ccc4c5cc6c7ccccc7c7ccccc7c6cc5n(-c5ccc6ccccc6c5)c4c3)c3cccc4cc5c(cc34)-c3ccccc3C53c4ccccc4-c4ccccc43)cc2)cc1]

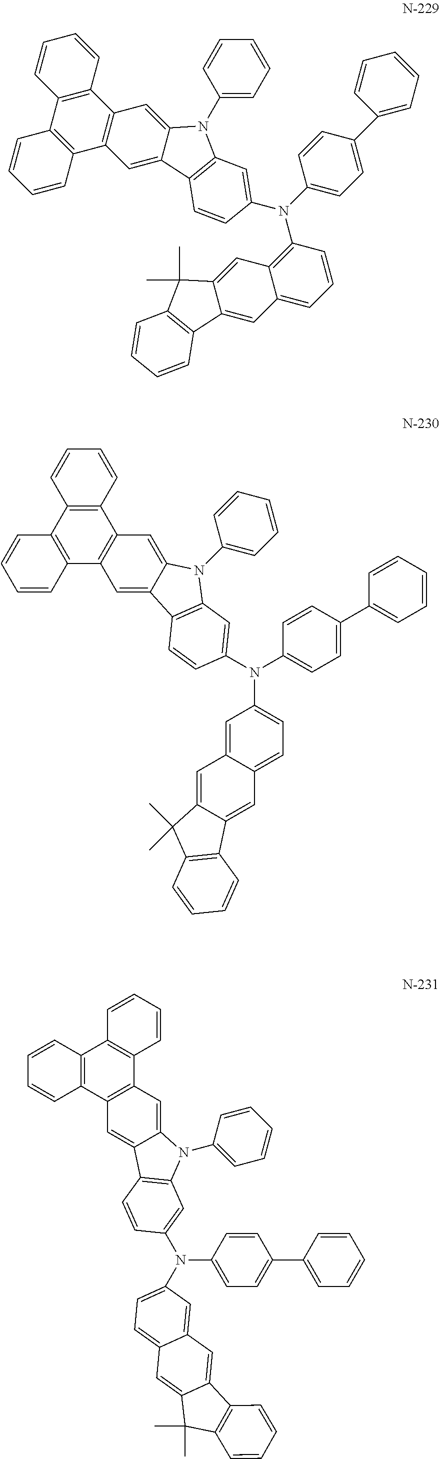 [SMILES: CC1(C)c2ccccc2-c2cc3cccc(N(c4ccc(-c5ccccc5)cc4)c4ccc5c6cc7c8ccccc8c8ccccc8c7cc6n(-c6ccccc6)c5c4)c3cc21 ; CC1(C)c2ccccc2-c2cc3ccc(N(c4ccc(-c5ccccc5)cc4)c4ccc5c6cc7c8ccccc8c8ccccc8c7cc6n(-c6ccccc6)c5c4)cc3cc21 ; CC1(C)c2ccccc2-c2cc3cc(N(c4ccc(-c5ccccc5)cc4)c4ccc5c6cc7c8ccccc8c8ccccc8c7cc6n(-c6ccccc6)c5c4)ccc3cc21]

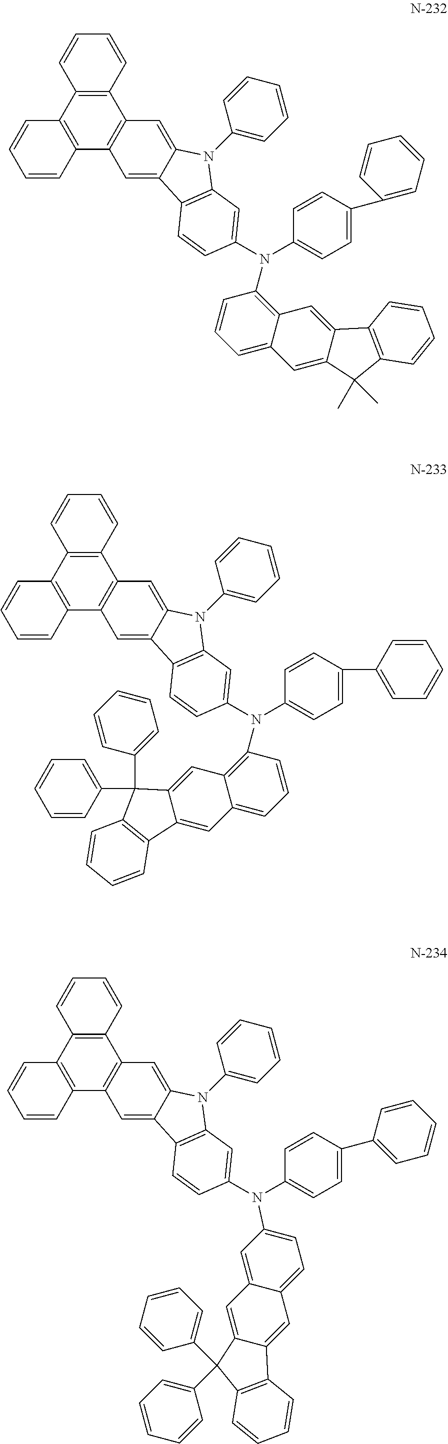 [SMILES: CC1(C)c2ccccc2-c2cc3c(N(c4ccc(-c5ccccc5)cc4)c4ccc5c6cc7c8ccccc8c8ccccc8c7cc6n(-c6ccccc6)c5c4)cccc3cc21 ; c1ccc(-c2ccc(N(c3ccc4c5cc6c7ccccc7c7ccccc7c6cc5n(-c5ccccc5)c4c3)c3cccc4cc5c(cc34)C(c3ccccc3)(c3ccccc3)c3ccccc3-5)cc2)cc1 ; c1ccc(-c2ccc(N(c3ccc4cc5c(cc4c3)C(c3ccccc3)(c3ccccc3)c3ccccc3-5)c3ccc4c5cc6c7ccccc7c7ccccc7c6cc5n(-c5ccccc5)c4c3)cc2)cc1]

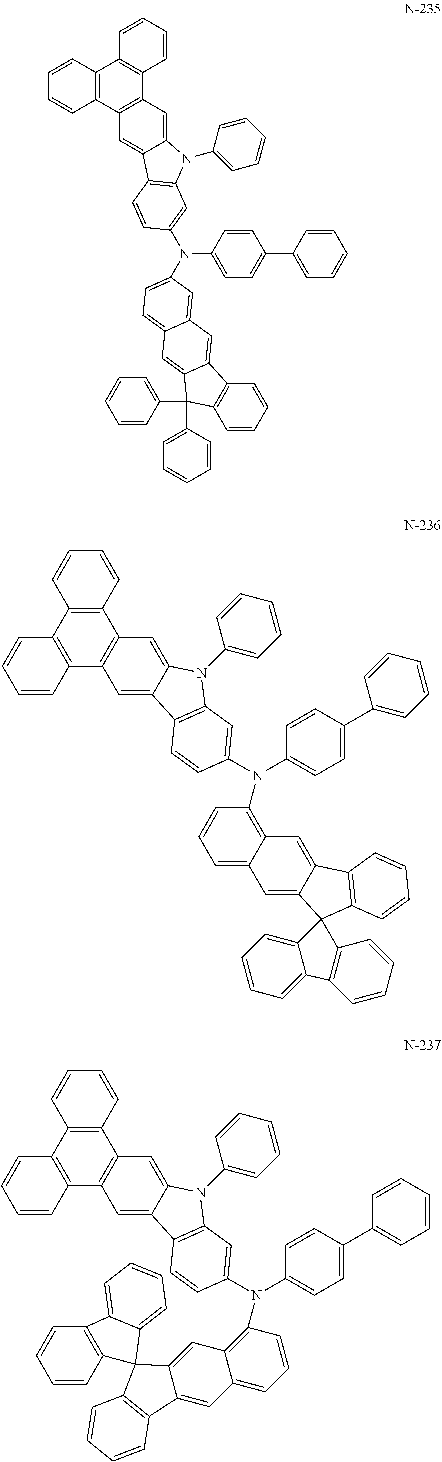 [SMILES: c1ccc(-c2ccc(N(c3ccc4cc5c(cc4c3)-c3ccccc3C5(c3ccccc3)c3ccccc3)c3ccc4c5cc6c7ccccc7c7ccccc7c6cc5n(-c5ccccc5)c4c3)cc2)cc1 ; c1ccc(-c2ccc(N(c3ccc4c5cc6c7ccccc7c7ccccc7c6cc5n(-c5ccccc5)c4c3)c3cccc4cc5c(cc34)-c3ccccc3C53c4ccccc4-c4ccccc43)cc2)cc1 ; c1ccc(-c2ccc(N(c3ccc4c5cc6c7ccccc7c7ccccc7c6cc5n(-c5ccccc5)c4c3)c3cccc4cc5c(cc34)C3(c4ccccc4-c4ccccc43)c3ccccc3-5)cc2)cc1]

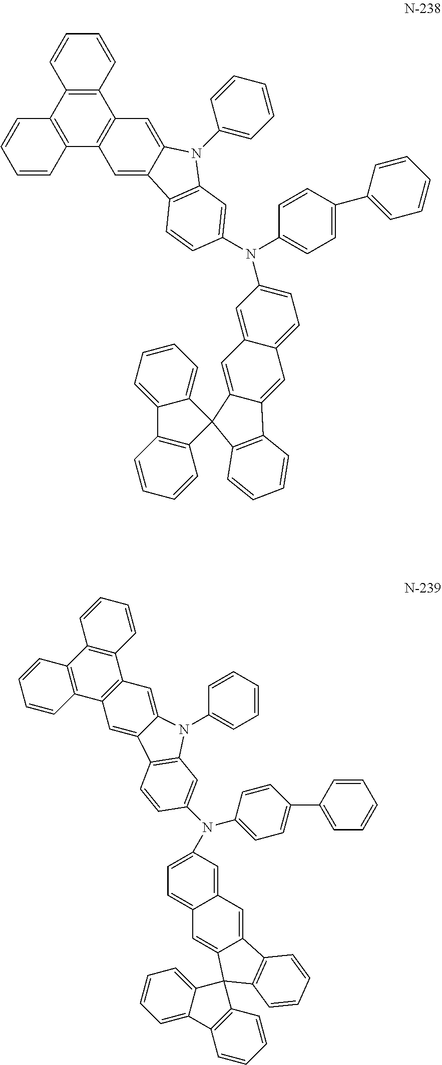 [SMILES: c1ccc(-c2ccc(N(c3ccc4cc5c(cc4c3)C3(c4ccccc4-c4ccccc43)c3ccccc3-5)c3ccc4c5cc6c7ccccc7c7ccccc7c6cc5n(-c5ccccc5)c4c3)cc2)cc1 ; c1ccc(-c2ccc(N(c3ccc4cc5c(cc4c3)-c3ccccc3C53c4ccccc4-c4ccccc43)c3ccc4c5cc6c7ccccc7c7ccccc7c6cc5n(-c5ccccc5)c4c3)cc2)cc1]

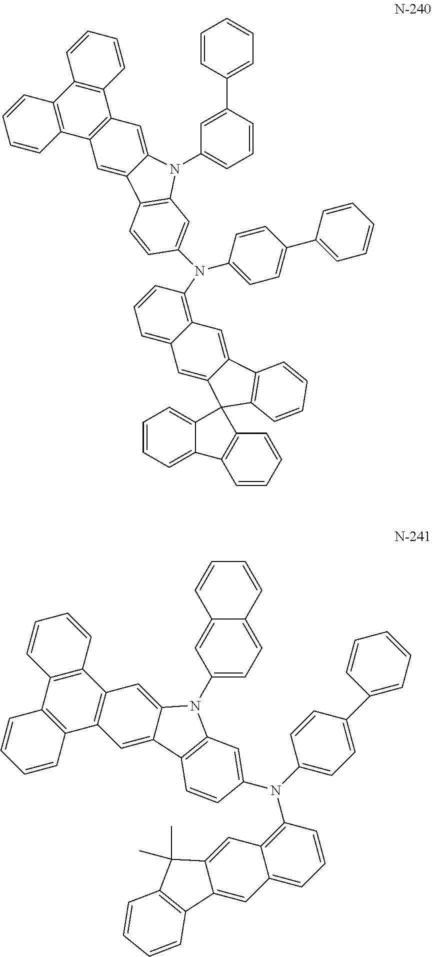 [SMILES: c1ccc(-c2ccc(N(c3ccc4c5cc6c7ccccc7c7ccccc7c6cc5n(-c5cccc(-c6ccccc6)c5)c4c3)c3cccc4cc5c(cc34)-c3ccccc3C53c4ccccc4-c4ccccc43)cc2)cc1 ; CC1(C)c2ccccc2-c2cc3cccc(N(c4ccc(-c5ccccc5)cc4)c4ccc5c6cc7c8ccccc8c8ccccc8c7cc6n(-c6ccc7ccccc7c6)c5c4)c3cc21]

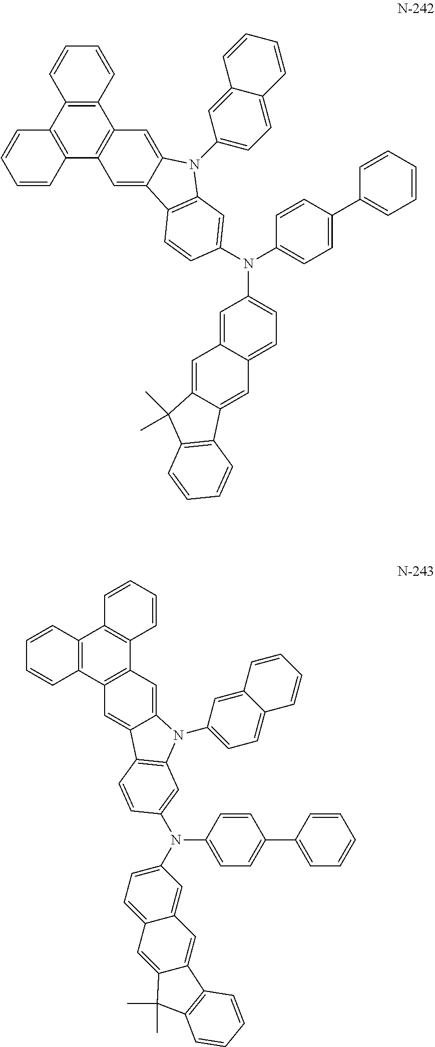 [SMILES: CC1(C)c2ccccc2-c2cc3ccc(N(c4ccc(-c5ccccc5)cc4)c4ccc5c6cc7c8ccccc8c8ccccc8c7cc6n(-c6ccc7ccccc7c6)c5c4)cc3cc21 ; CC1(C)c2ccccc2-c2cc3cc(N(c4ccc(-c5ccccc5)cc4)c4ccc5c6cc7c8ccccc8c8ccccc8c7cc6n(-c6ccc7ccccc7c6)c5c4)ccc3cc21]

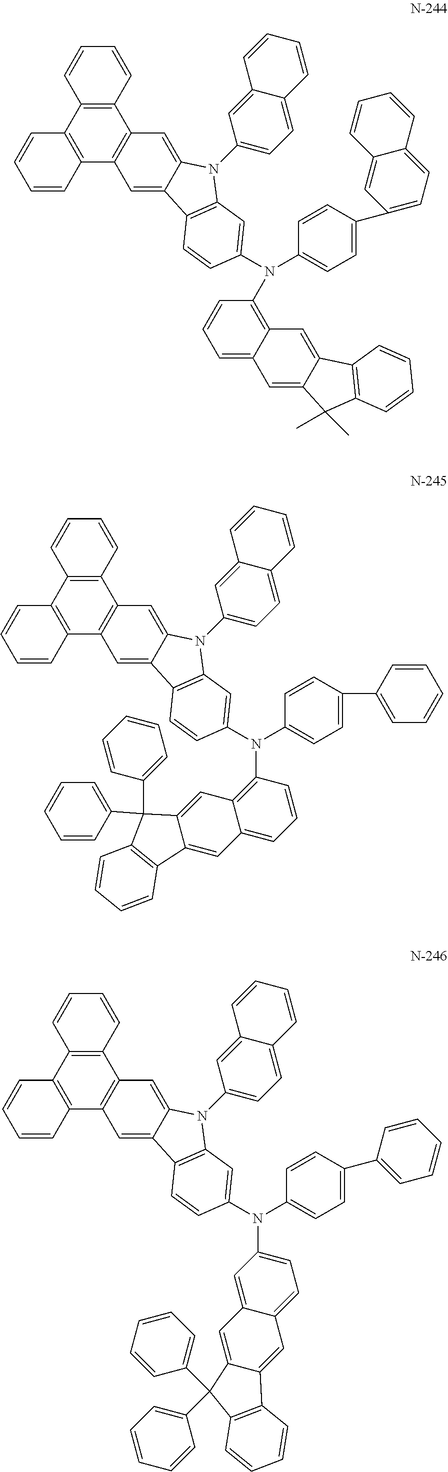 [SMILES: CC1(C)c2ccccc2-c2cc3c(N(c4ccc(-c5ccc6ccccc6c5)cc4)c4ccc5c6cc7c8ccccc8c8ccccc8c7cc6n(-c6ccc7ccccc7c6)c5c4)cccc3cc21 ; c1ccc(-c2ccc(N(c3ccc4c5cc6c7ccccc7c7ccccc7c6cc5n(-c5ccc6ccccc6c5)c4c3)c3cccc4cc5c(cc34)C(c3ccccc3)(c3ccccc3)c3ccccc3-5)cc2)cc1 ; c1ccc(-c2ccc(N(c3ccc4cc5c(cc4c3)C(c3ccccc3)(c3ccccc3)c3ccccc3-5)c3ccc4c5cc6c7ccccc7c7ccccc7c6cc5n(-c5ccc6ccccc6c5)c4c3)cc2)cc1]

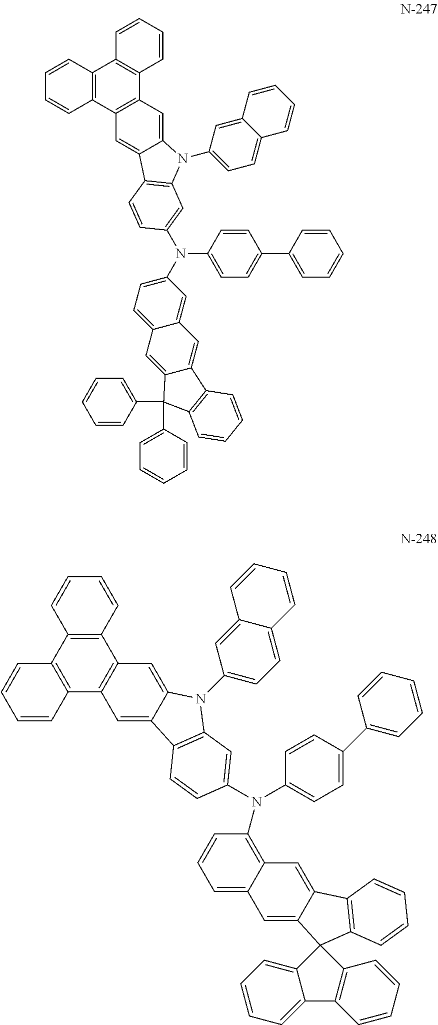 [SMILES: c1ccc(-c2ccc(N(c3ccc4cc5c(cc4c3)-c3ccccc3C5(c3ccccc3)c3ccccc3)c3ccc4c5cc6c7ccccc7c7ccccc7c6cc5n(-c5ccc6ccccc6c5)c4c3)cc2)cc1 ; c1ccc(-c2ccc(N(c3ccc4c5cc6c7ccccc7c7ccccc7c6cc5n(-c5ccc6ccccc6c5)c4c3)c3cccc4cc5c(cc34)-c3ccccc3C53c4ccccc4-c4ccccc43)cc2)cc1]

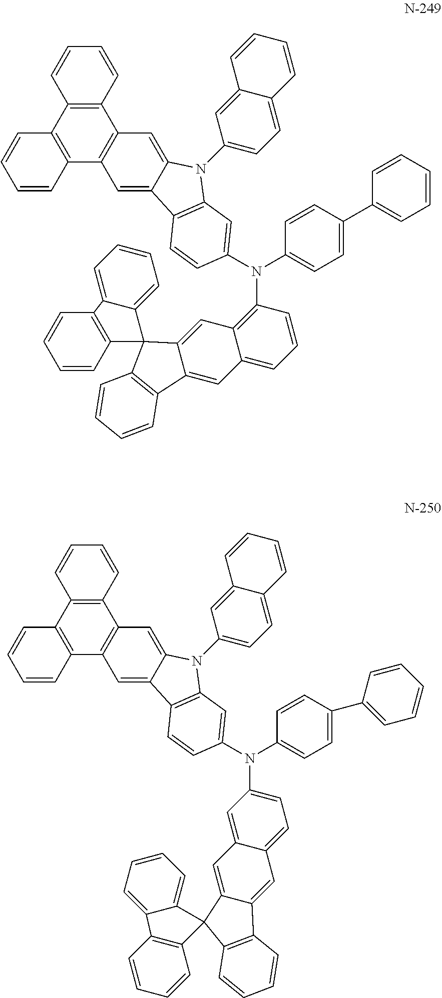 [SMILES: c1ccc(-c2ccc(N(c3ccc4c5cc6c7ccccc7c7ccccc7c6cc5n(-c5ccc6ccccc6c5)c4c3)c3cccc4cc5c(cc34)C3(c4ccccc4-c4ccccc43)c3ccccc3-5)cc2)cc1 ; c1ccc(-c2ccc(N(c3ccc4cc5c(cc4c3)C3(c4ccccc4-c4ccccc43)c3ccccc3-5)c3ccc4c5cc6c7ccccc7c7ccccc7c6cc5n(-c5ccc6ccccc6c5)c4c3)cc2)cc1]

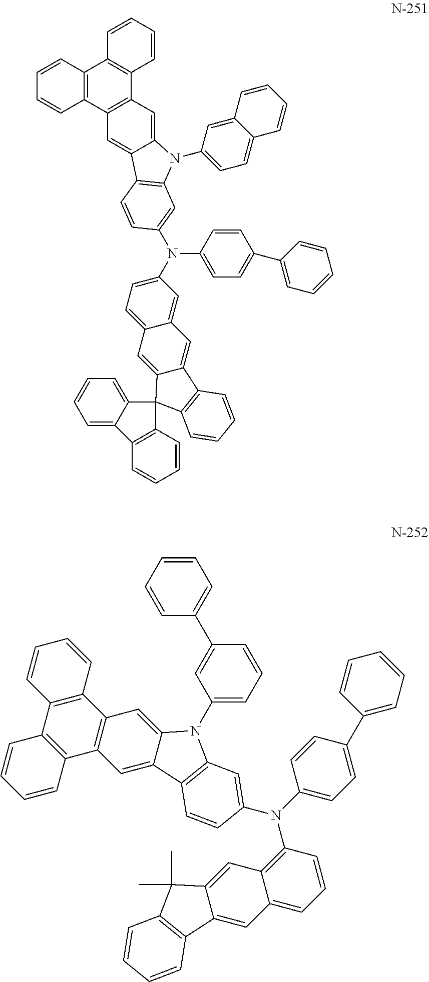 [SMILES: c1ccc(-c2ccc(N(c3ccc4cc5c(cc4c3)-c3ccccc3C53c4ccccc4-c4ccccc43)c3ccc4c5cc6c7ccccc7c7ccccc7c6cc5n(-c5ccc6ccccc6c5)c4c3)cc2)cc1 ; CC1(C)c2ccccc2-c2cc3cccc(N(c4ccc(-c5ccccc5)cc4)c4ccc5c6cc7c8ccccc8c8ccccc8c7cc6n(-c6cccc(-c7ccccc7)c6)c5c4)c3cc21]

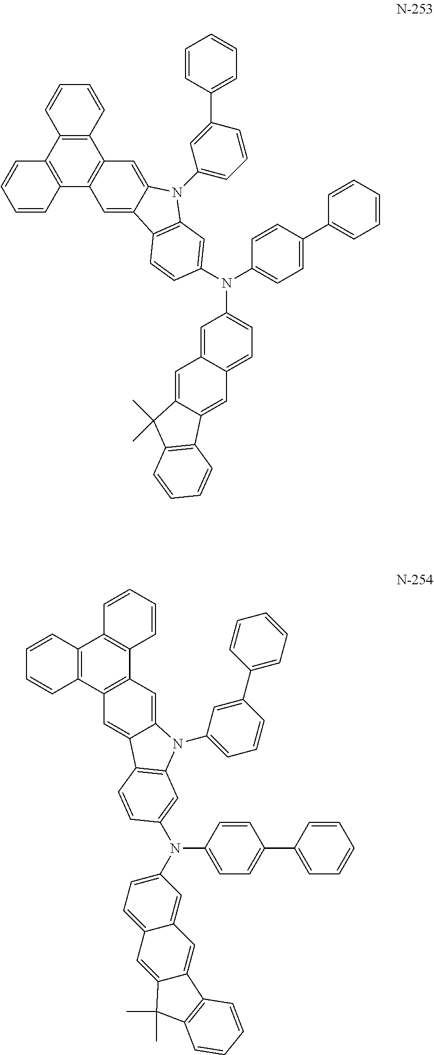 [SMILES: CC1(C)c2ccccc2-c2cc3ccc(N(c4ccc(-c5ccccc5)cc4)c4ccc5c6cc7c8ccccc8c8ccccc8c7cc6n(-c6cccc(-c7ccccc7)c6)c5c4)cc3cc21 ; CC1(C)c2ccccc2-c2cc3cc(N(c4ccc(-c5ccccc5)cc4)c4ccc5c6cc7c8ccccc8c8ccccc8c7cc6n(-c6cccc(-c7ccccc7)c6)c5c4)ccc3cc21]

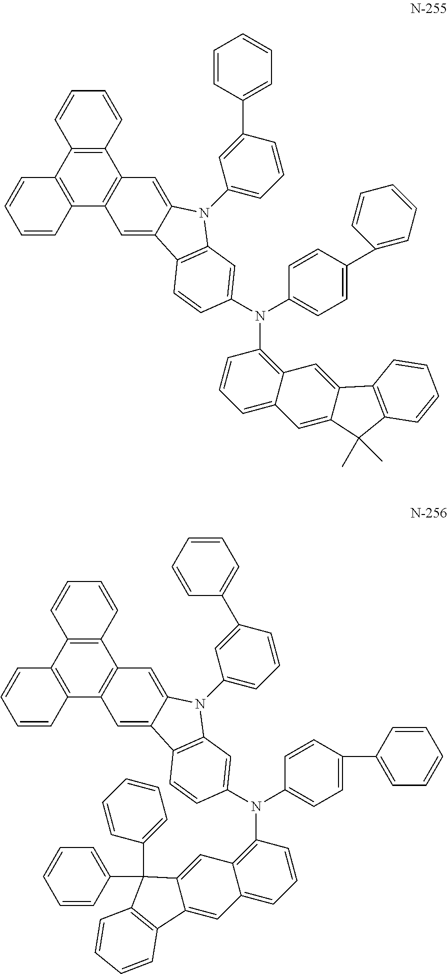 [SMILES: CC1(C)c2ccccc2-c2cc3c(N(c4ccc(-c5ccccc5)cc4)c4ccc5c6cc7c8ccccc8c8ccccc8c7cc6n(-c6cccc(-c7ccccc7)c6)c5c4)cccc3cc21 ; c1ccc(-c2ccc(N(c3ccc4c5cc6c7ccccc7c7ccccc7c6cc5n(-c5cccc(-c6ccccc6)c5)c4c3)c3cccc4cc5c(cc34)C(c3ccccc3)(c3ccccc3)c3ccccc3-5)cc2)cc1]

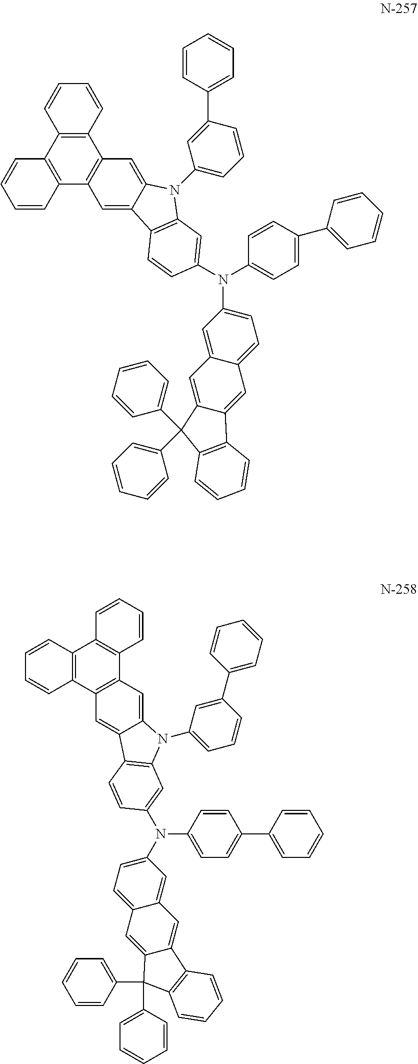 [SMILES: c1ccc(-c2ccc(N(c3ccc4cc5c(cc4c3)C(c3ccccc3)(c3ccccc3)c3ccccc3-5)c3ccc4c5cc6c7ccccc7c7ccccc7c6cc5n(-c5cccc(-c6ccccc6)c5)c4c3)cc2)cc1 ; c1ccc(-c2ccc(N(c3ccc4cc5c(cc4c3)-c3ccccc3C5(c3ccccc3)c3ccccc3)c3ccc4c5cc6c7ccccc7c7ccccc7c6cc5n(-c5cccc(-c6ccccc6)c5)c4c3)cc2)cc1]

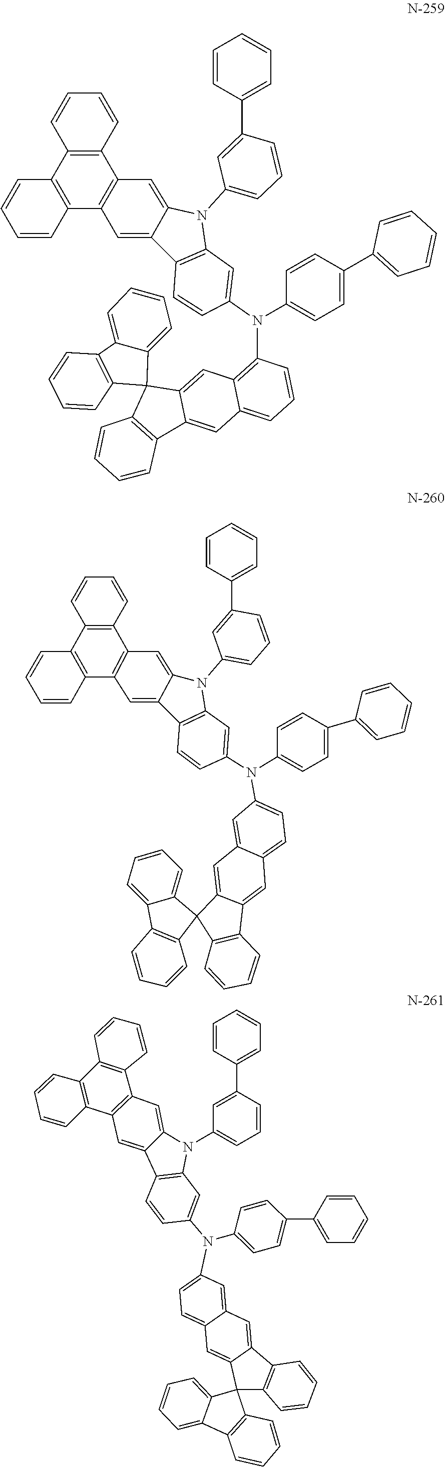

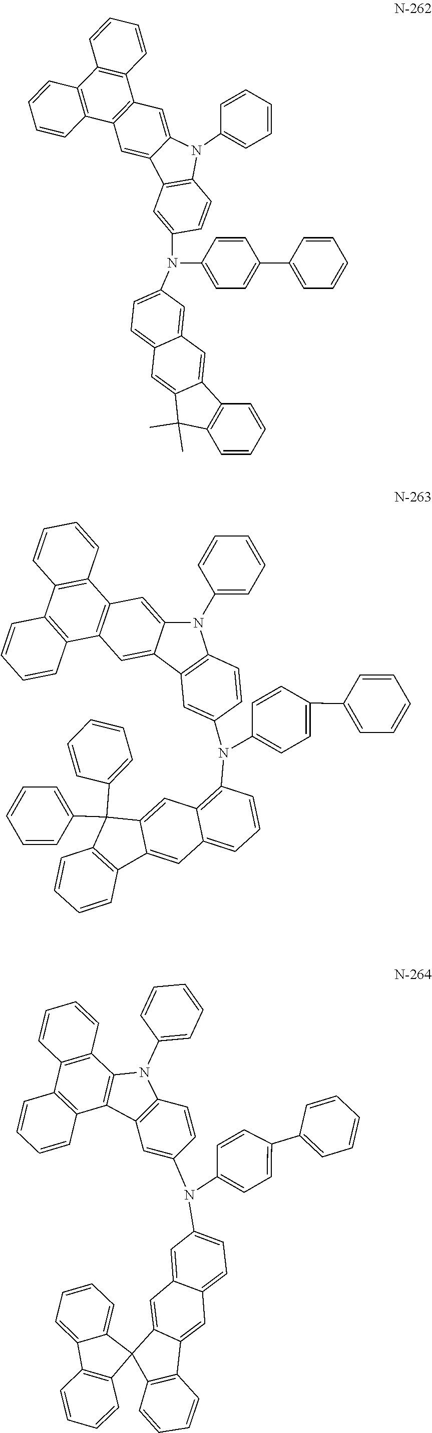 [SMILES: CC1(C)c2ccccc2-c2cc3cc(N(c4ccc(-c5ccccc5)cc4)c4ccc5c(c4)c4cc6c7ccccc7c7ccccc7c6cc4n5-c4ccccc4)ccc3cc21 ; c1ccc(-c2ccc(N(c3ccc4c(c3)c3cc5c6ccccc6c6ccccc6c5cc3n4-c3ccccc3)c3cccc4cc5c(cc34)C(c3ccccc3)(c3ccccc3)c3ccccc3-5)cc2)cc1 ; c1ccc(-c2ccc(N(c3ccc4cc5c(cc4c3)C3(c4ccccc4-c4ccccc43)c3ccccc3-5)c3ccc4c(c3)c3c5ccccc5c5ccccc5c3n4-c3ccccc3)cc2)cc1]

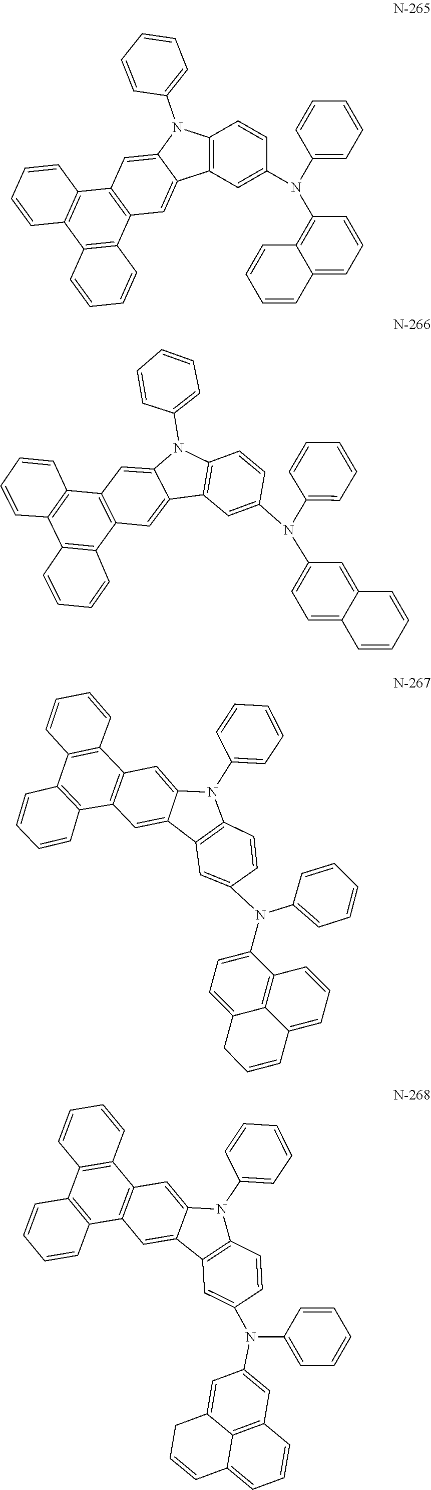 [SMILES: c1ccc(N(c2ccc3c(c2)c2cc4c5ccccc5c5ccccc5c4cc2n3-c2ccccc2)c2cccc3ccccc23)cc1 ; c1ccc(N(c2ccc3ccccc3c2)c2ccc3c(c2)c2cc4c5ccccc5c5ccccc5c4cc2n3-c2ccccc2)cc1 ; C1=Cc2cccc3c(N(c4ccccc4)c4ccc5c(c4)c4cc6c7ccccc7c7ccccc7c6cc4n5-c4ccccc4)ccc(c23)C1 ; C1=Cc2cccc3cc(N(c4ccccc4)c4ccc5c(c4)c4cc6c7ccccc7c7ccccc7c6cc4n5-c4ccccc4)cc(c23)C1]

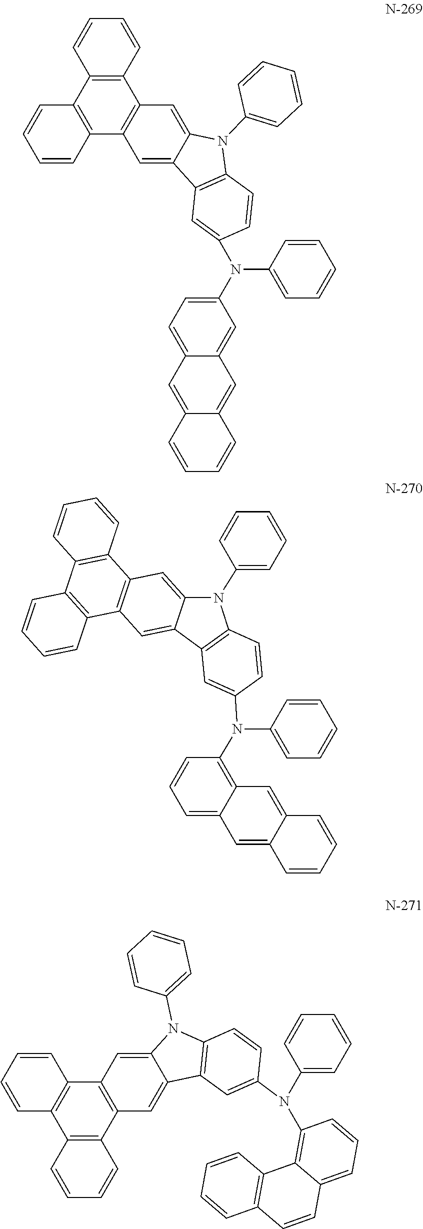

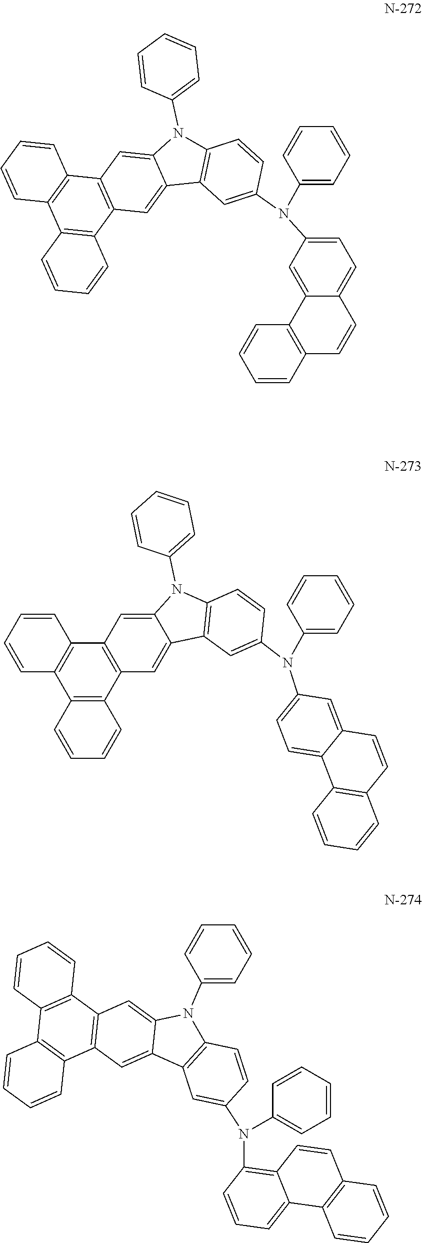 [SMILES: c1ccc(N(c2ccc3ccc4ccccc4c3c2)c2ccc3c(c2)c2cc4c5ccccc5c5ccccc5c4cc2n3-c2ccccc2)cc1 ; c1ccc(N(c2ccc3c(ccc4ccccc43)c2)c2ccc3c(c2)c2cc4c5ccccc5c5ccccc5c4cc2n3-c2ccccc2)cc1 ; c1ccc(N(c2ccc3c(c2)c2cc4c5ccccc5c5ccccc5c4cc2n3-c2ccccc2)c2cccc3c2ccc2ccccc23)cc1]

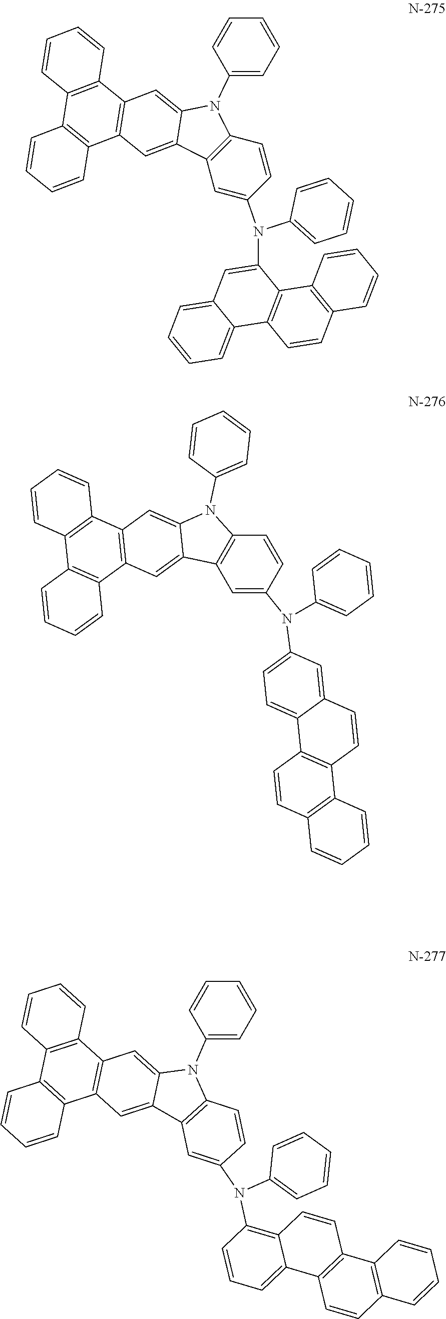 [SMILES: c1ccc(N(c2ccc3c(c2)c2cc4c5ccccc5c5ccccc5c4cc2n3-c2ccccc2)c2cc3ccccc3c3ccc4ccccc4c23)cc1 ; c1ccc(N(c2ccc3c(ccc4c5ccccc5ccc34)c2)c2ccc3c(c2)c2cc4c5ccccc5c5ccccc5c4cc2n3-c2ccccc2)cc1 ; c1ccc(N(c2ccc3c(c2)c2cc4c5ccccc5c5ccccc5c4cc2n3-c2ccccc2)c2cccc3c2ccc2c4ccccc4ccc32)cc1]

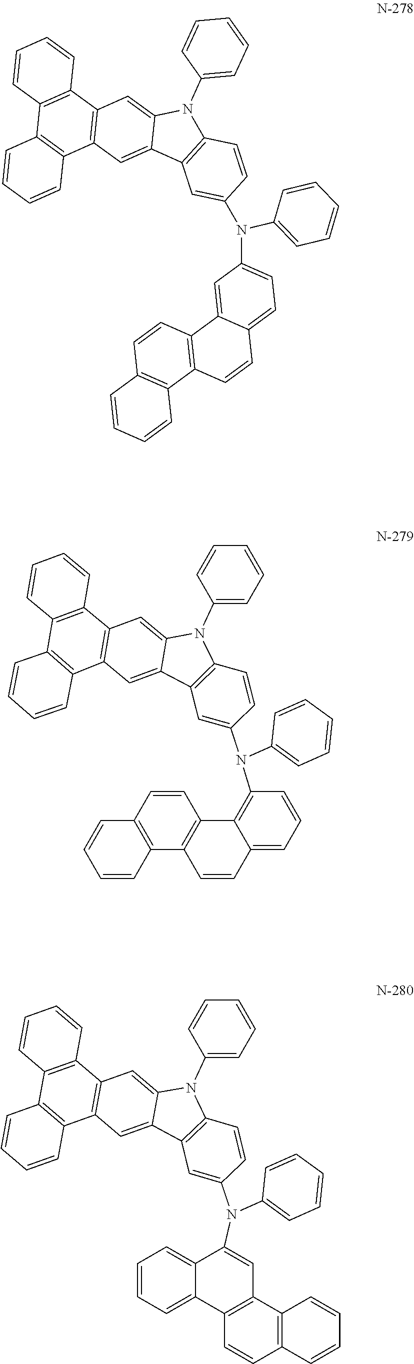 [SMILES: c1ccc(N(c2ccc3ccc4c5ccccc5ccc4c3c2)c2ccc3c(c2)c2cc4c5ccccc5c5ccccc5c4cc2n3-c2ccccc2)cc1 ; c1ccc(N(c2ccc3c(c2)c2cc4c5ccccc5c5ccccc5c4cc2n3-c2ccccc2)c2cccc3ccc4c5ccccc5ccc4c23)cc1 ; c1ccc(N(c2ccc3c(c2)c2cc4c5ccccc5c5ccccc5c4cc2n3-c2ccccc2)c2cc3c4ccccc4ccc3c3ccccc23)cc1]

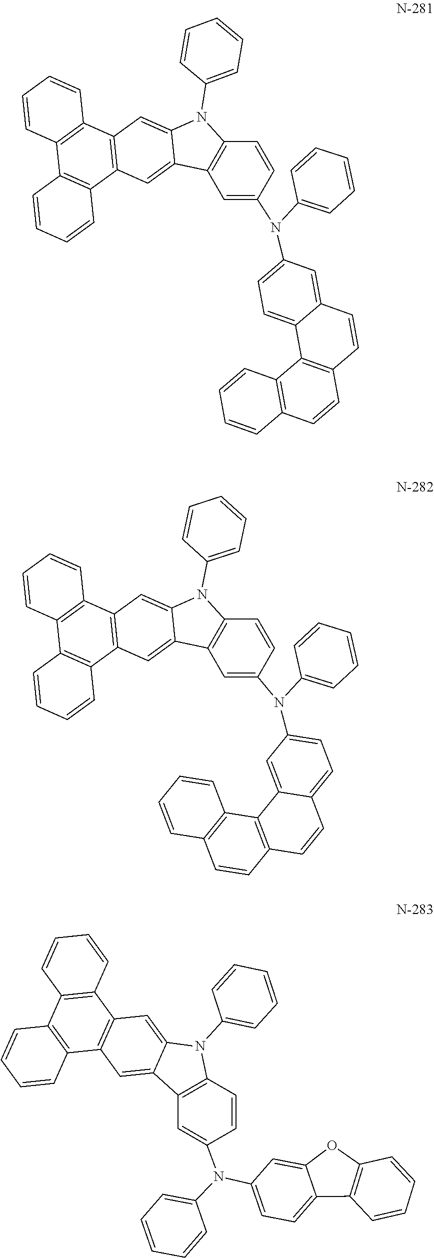 [SMILES: c1ccc(N(c2ccc3c(ccc4ccc5ccccc5c43)c2)c2ccc3c(c2)c2cc4c5ccccc5c5ccccc5c4cc2n3-c2ccccc2)cc1 ; c1ccc(N(c2ccc3ccc4ccc5ccccc5c4c3c2)c2ccc3c(c2)c2cc4c5ccccc5c5ccccc5c4cc2n3-c2ccccc2)cc1 ; c1ccc(N(c2ccc3c(c2)oc2ccccc23)c2ccc3c(c2)c2cc4c5ccccc5c5ccccc5c4cc2n3-c2ccccc2)cc1]

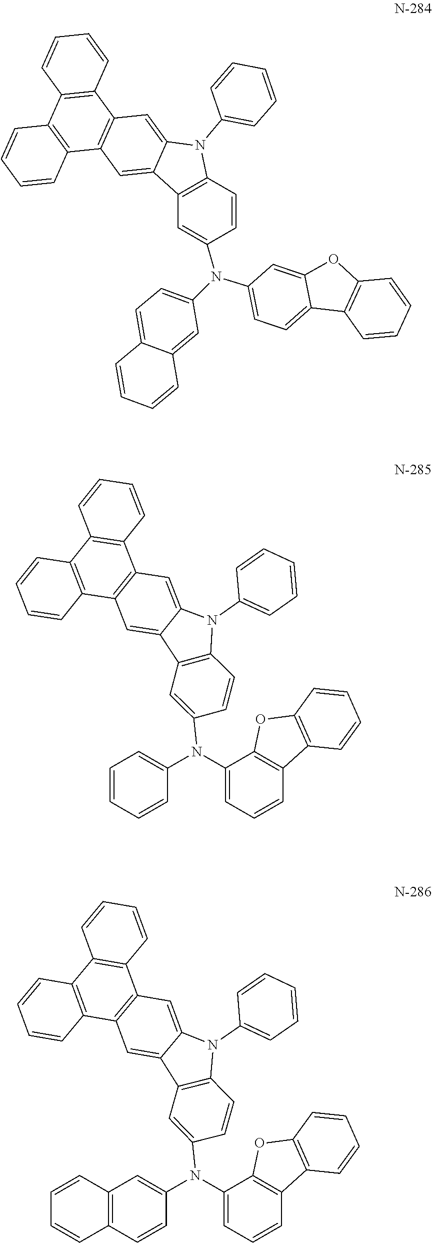 [SMILES: c1ccc(-n2c3ccc(N(c4ccc5ccccc5c4)c4ccc5c(c4)oc4ccccc45)cc3c3cc4c5ccccc5c5ccccc5c4cc32)cc1 ; c1ccc(N(c2ccc3c(c2)c2cc4c5ccccc5c5ccccc5c4cc2n3-c2ccccc2)c2cccc3c2oc2ccccc23)cc1 ; c1ccc(-n2c3ccc(N(c4ccc5ccccc5c4)c4cccc5c4oc4ccccc45)cc3c3cc4c5ccccc5c5ccccc5c4cc32)cc1]

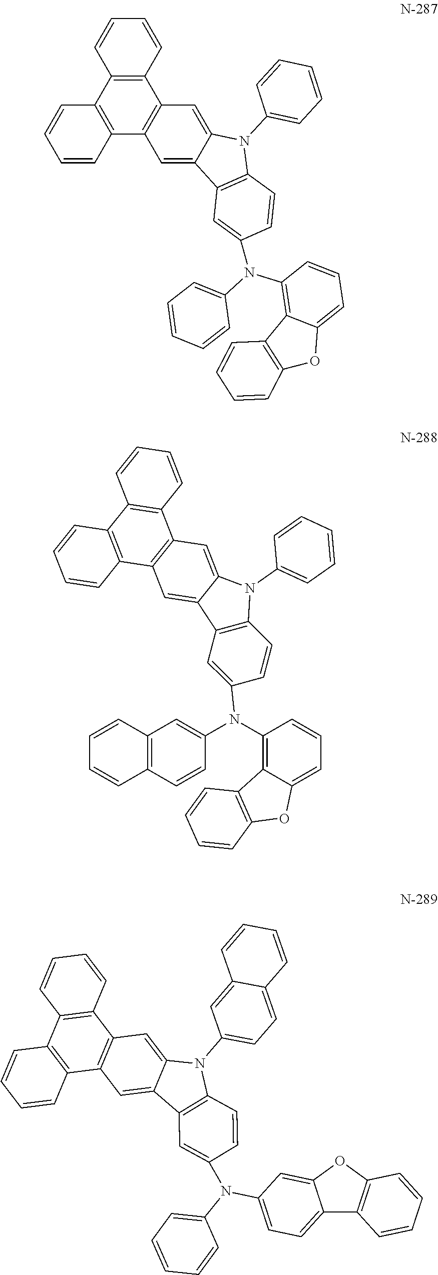 [SMILES: c1ccc(N(c2ccc3c(c2)c2cc4c5ccccc5c5ccccc5c4cc2n3-c2ccccc2)c2cccc3oc4ccccc4c23)cc1 ; c1ccc(-n2c3ccc(N(c4ccc5ccccc5c4)c4cccc5oc6ccccc6c45)cc3c3cc4c5ccccc5c5ccccc5c4cc32)cc1 ; c1ccc(N(c2ccc3c(c2)oc2ccccc23)c2ccc3c(c2)c2cc4c5ccccc5c5ccccc5c4cc2n3-c2ccc3ccccc3c2)cc1]

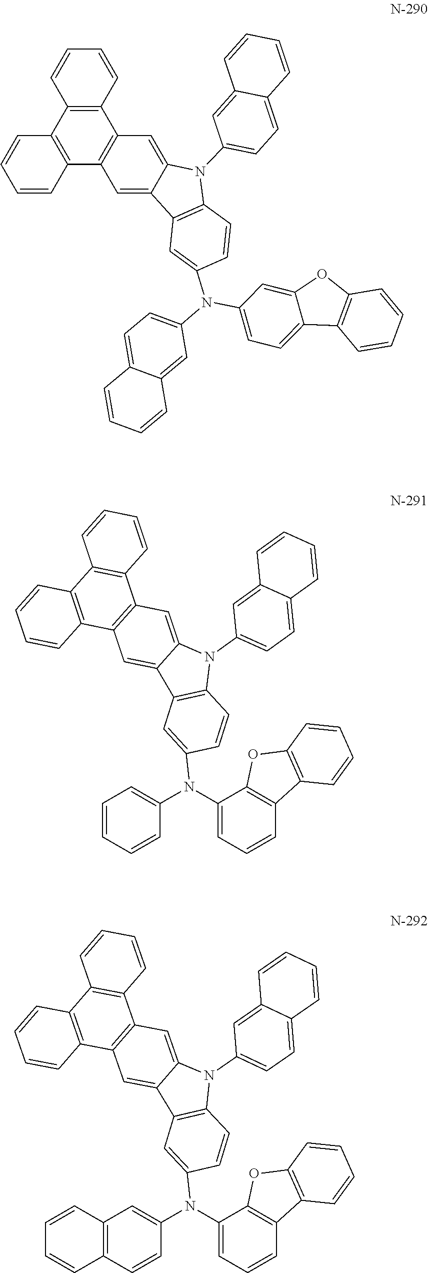 [SMILES: c1ccc2cc(N(c3ccc4c(c3)oc3ccccc34)c3ccc4c(c3)c3cc5c6ccccc6c6ccccc6c5cc3n4-c3ccc4ccccc4c3)ccc2c1 ; c1ccc(N(c2ccc3c(c2)c2cc4c5ccccc5c5ccccc5c4cc2n3-c2ccc3ccccc3c2)c2cccc3c2oc2ccccc23)cc1 ; c1ccc2cc(N(c3ccc4c(c3)c3cc5c6ccccc6c6ccccc6c5cc3n4-c3ccc4ccccc4c3)c3cccc4c3oc3ccccc34)ccc2c1]

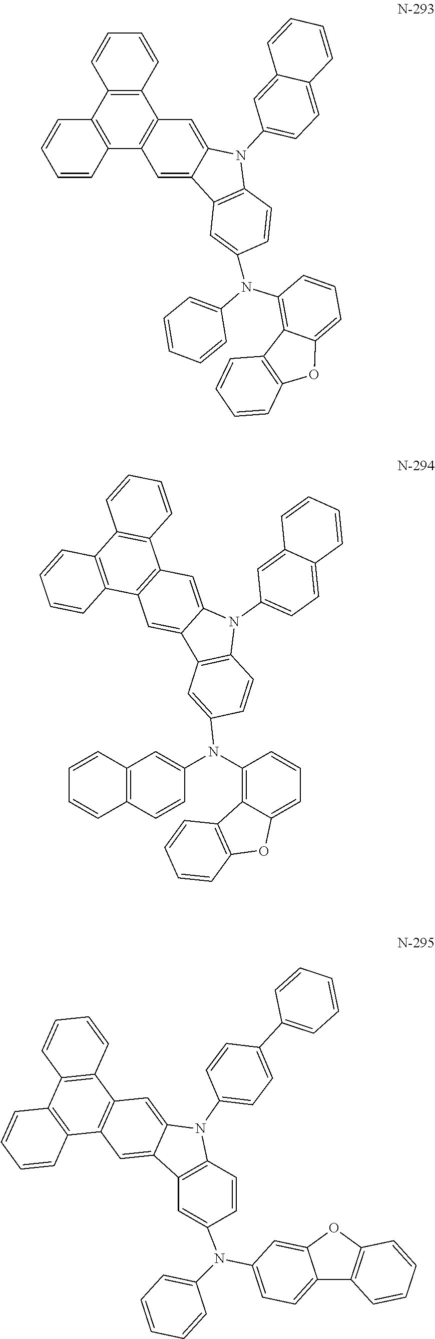 [SMILES: c1ccc(N(c2ccc3c(c2)c2cc4c5ccccc5c5ccccc5c4cc2n3-c2ccc3ccccc3c2)c2cccc3oc4ccccc4c23)cc1 ; c1ccc2cc(N(c3ccc4c(c3)c3cc5c6ccccc6c6ccccc6c5cc3n4-c3ccc4ccccc4c3)c3cccc4oc5ccccc5c34)ccc2c1 ; c1ccc(-c2ccc(-n3c4ccc(N(c5ccccc5)c5ccc6c(c5)oc5ccccc56)cc4c4cc5c6ccccc6c6ccccc6c5cc43)cc2)cc1]

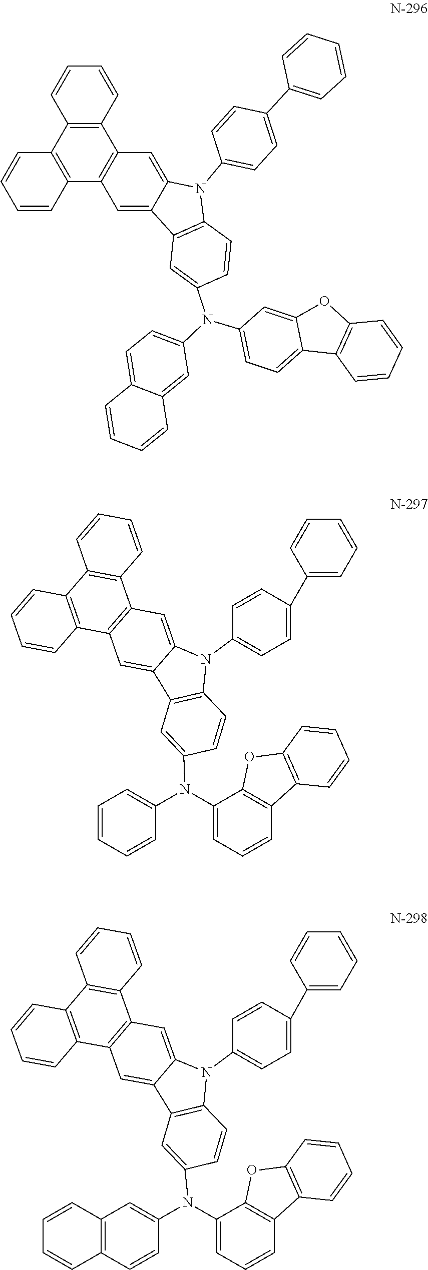 [SMILES: c1ccc(-c2ccc(-n3c4ccc(N(c5ccc6ccccc6c5)c5ccc6c(c5)oc5ccccc56)cc4c4cc5c6ccccc6c6ccccc6c5cc43)cc2)cc1 ; c1ccc(-c2ccc(-n3c4ccc(N(c5ccccc5)c5cccc6c5oc5ccccc56)cc4c4cc5c6ccccc6c6ccccc6c5cc43)cc2)cc1 ; c1ccc(-c2ccc(-n3c4ccc(N(c5ccc6ccccc6c5)c5cccc6c5oc5ccccc56)cc4c4cc5c6ccccc6c6ccccc6c5cc43)cc2)cc1]

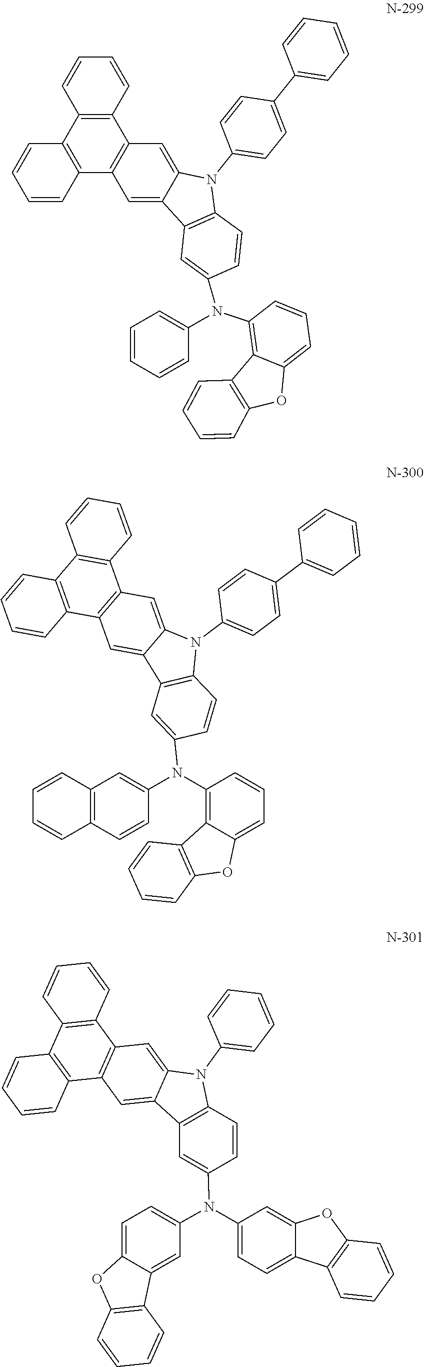 [SMILES: c1ccc(-c2ccc(-n3c4ccc(N(c5ccccc5)c5cccc6oc7ccccc7c56)cc4c4cc5c6ccccc6c6ccccc6c5cc43)cc2)cc1 ; c1ccc(-c2ccc(-n3c4ccc(N(c5ccc6ccccc6c5)c5cccc6oc7ccccc7c56)cc4c4cc5c6ccccc6c6ccccc6c5cc43)cc2)cc1 ; c1ccc(-n2c3ccc(N(c4ccc5c(c4)oc4ccccc45)c4ccc5oc6ccccc6c5c4)cc3c3cc4c5ccccc5c5ccccc5c4cc32)cc1]

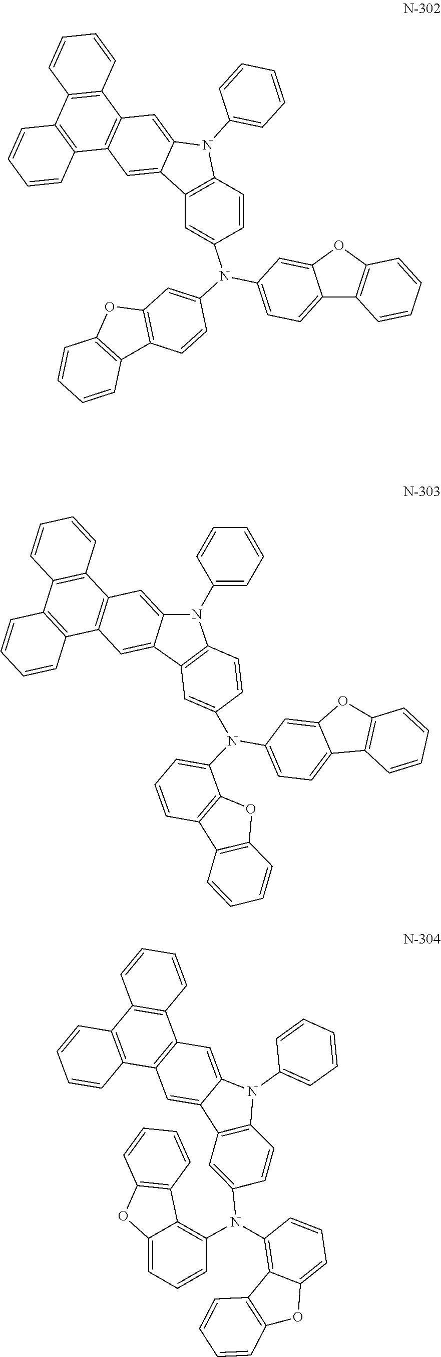 [SMILES: c1ccc(-n2c3ccc(N(c4ccc5c(c4)oc4ccccc45)c4ccc5c(c4)oc4ccccc45)cc3c3cc4c5ccccc5c5ccccc5c4cc32)cc1 ; c1ccc(-n2c3ccc(N(c4ccc5c(c4)oc4ccccc45)c4cccc5c4oc4ccccc45)cc3c3cc4c5ccccc5c5ccccc5c4cc32)cc1 ; c1ccc(-n2c3ccc(N(c4cccc5oc6ccccc6c45)c4cccc5oc6ccccc6c45)cc3c3cc4c5ccccc5c5ccccc5c4cc32)cc1]

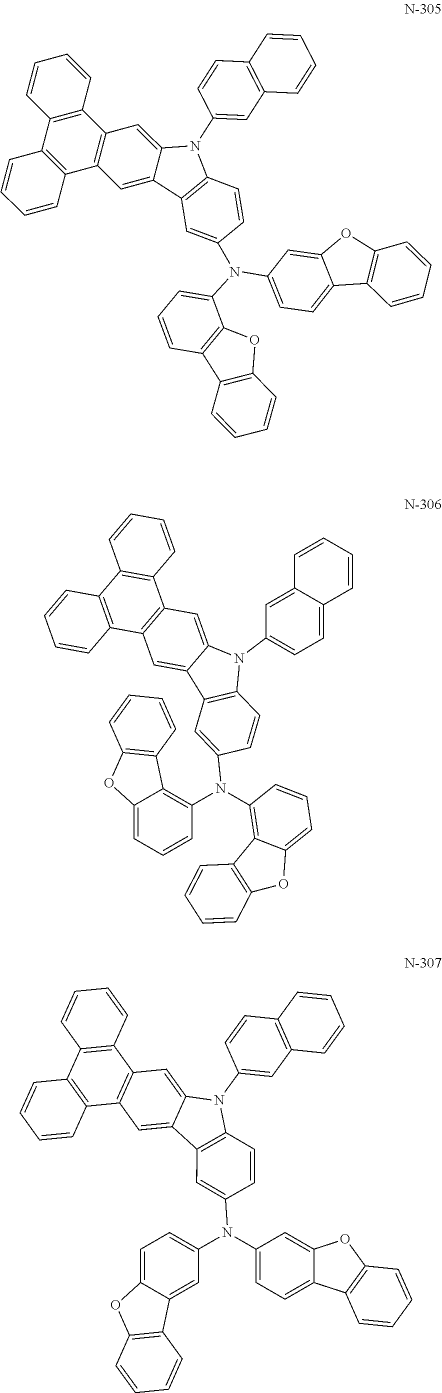 [SMILES: c1ccc2cc(-n3c4ccc(N(c5ccc6c(c5)oc5ccccc56)c5cccc6c5oc5ccccc56)cc4c4cc5c6ccccc6c6ccccc6c5cc43)ccc2c1 ; c1ccc2cc(-n3c4ccc(N(c5cccc6oc7ccccc7c56)c5cccc6oc7ccccc7c56)cc4c4cc5c6ccccc6c6ccccc6c5cc43)ccc2c1 ; c1ccc2cc(-n3c4ccc(N(c5ccc6c(c5)oc5ccccc56)c5ccc6oc7ccccc7c6c5)cc4c4cc5c6ccccc6c6ccccc6c5cc43)ccc2c1]

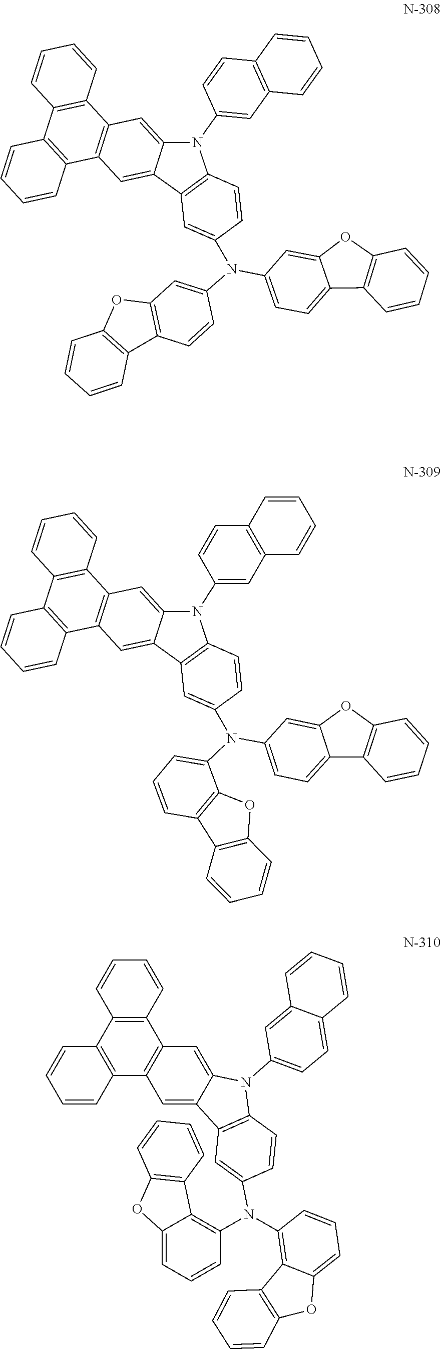 [SMILES: c1ccc2cc(-n3c4ccc(N(c5ccc6c(c5)oc5ccccc56)c5ccc6c(c5)oc5ccccc56)cc4c4cc5c6ccccc6c6ccccc6c5cc43)ccc2c1 ; c1ccc2cc(-n3c4ccc(N(c5ccc6c(c5)oc5ccccc56)c5cccc6c5oc5ccccc56)cc4c4cc5c6ccccc6c6ccccc6c5cc43)ccc2c1 ; c1ccc2cc(-n3c4ccc(N(c5cccc6oc7ccccc7c56)c5cccc6oc7ccccc7c56)cc4c4cc5c6ccccc6c6ccccc6c5cc43)ccc2c1]

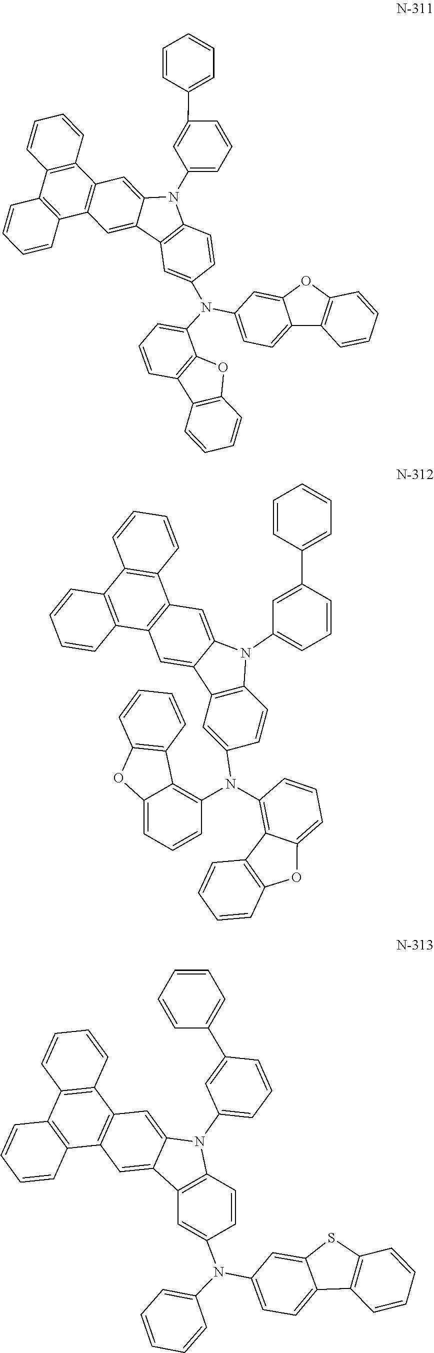 [SMILES: c1ccc(-c2cccc(-n3c4ccc(N(c5ccc6c(c5)oc5ccccc56)c5cccc6c5oc5ccccc56)cc4c4cc5c6ccccc6c6ccccc6c5cc43)c2)cc1 ; c1ccc(-c2cccc(-n3c4ccc(N(c5cccc6oc7ccccc7c56)c5cccc6oc7ccccc7c56)cc4c4cc5c6ccccc6c6ccccc6c5cc43)c2)cc1 ; c1ccc(-c2cccc(-n3c4ccc(N(c5ccccc5)c5ccc6c(c5)sc5ccccc56)cc4c4cc5c6ccccc6c6ccccc6c5cc43)c2)cc1]

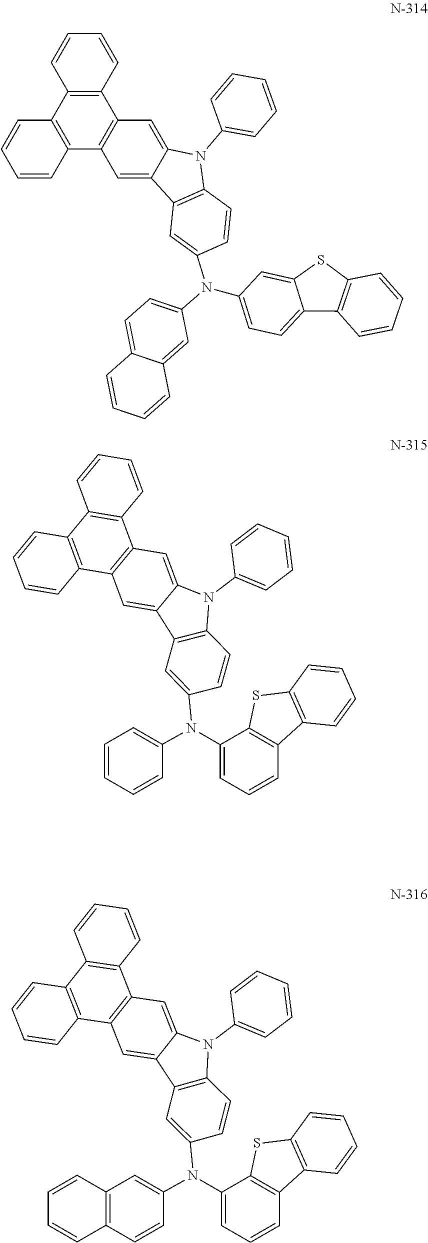

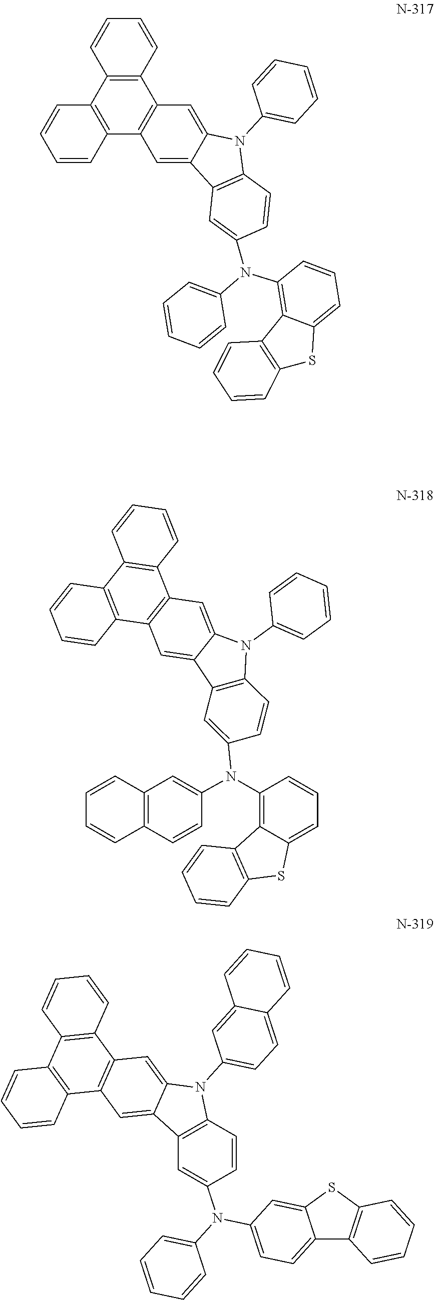 [SMILES: c1ccc(N(c2ccc3c(c2)c2cc4c5ccccc5c5ccccc5c4cc2n3-c2ccccc2)c2cccc3sc4ccccc4c23)cc1 ; c1ccc(-n2c3ccc(N(c4ccc5ccccc5c4)c4cccc5sc6ccccc6c45)cc3c3cc4c5ccccc5c5ccccc5c4cc32)cc1 ; c1ccc(N(c2ccc3c(c2)sc2ccccc23)c2ccc3c(c2)c2cc4c5ccccc5c5ccccc5c4cc2n3-c2ccc3ccccc3c2)cc1]

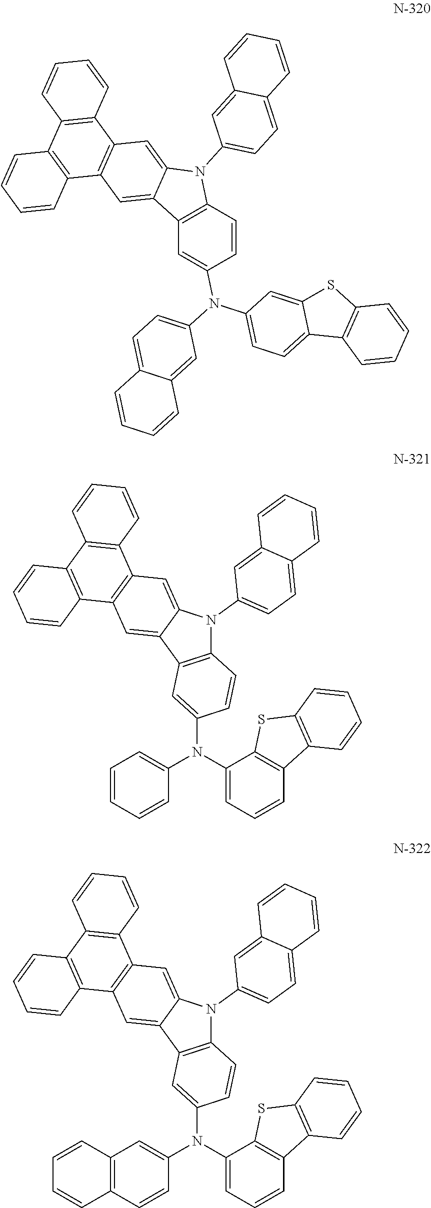 [SMILES: c1ccc2cc(N(c3ccc4c(c3)sc3ccccc34)c3ccc4c(c3)c3cc5c6ccccc6c6ccccc6c5cc3n4-c3ccc4ccccc4c3)ccc2c1 ; c1ccc(N(c2ccc3c(c2)c2cc4c5ccccc5c5ccccc5c4cc2n3-c2ccc3ccccc3c2)c2cccc3c2sc2ccccc23)cc1 ; c1ccc2cc(N(c3ccc4c(c3)c3cc5c6ccccc6c6ccccc6c5cc3n4-c3ccc4ccccc4c3)c3cccc4c3sc3ccccc34)ccc2c1]

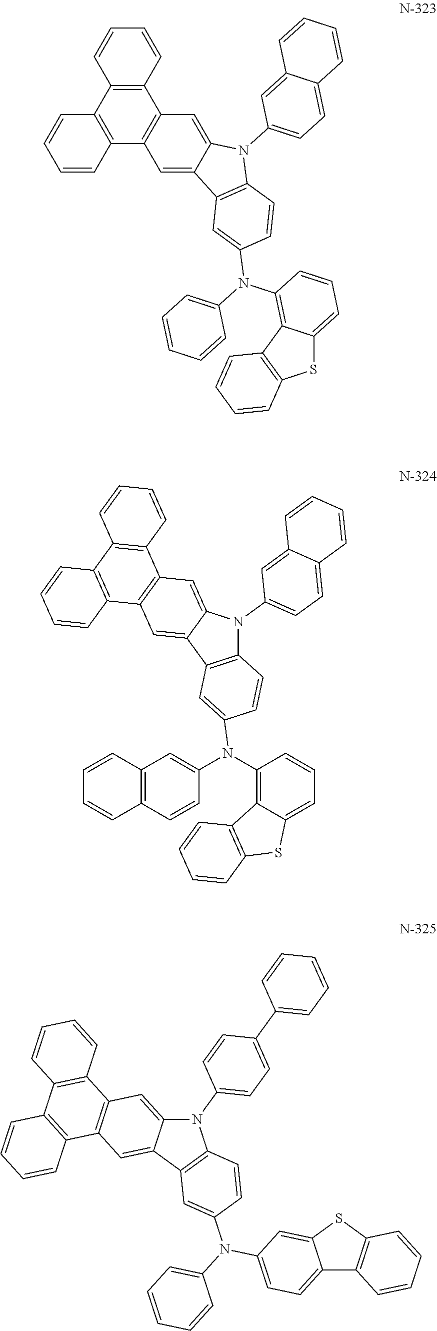 [SMILES: c1ccc(N(c2ccc3c(c2)c2cc4c5ccccc5c5ccccc5c4cc2n3-c2ccc3ccccc3c2)c2cccc3sc4ccccc4c23)cc1 ; c1ccc2cc(N(c3ccc4c(c3)c3cc5c6ccccc6c6ccccc6c5cc3n4-c3ccc4ccccc4c3)c3cccc4sc5ccccc5c34)ccc2c1 ; c1ccc(-c2ccc(-n3c4ccc(N(c5ccccc5)c5ccc6c(c5)sc5ccccc56)cc4c4cc5c6ccccc6c6ccccc6c5cc43)cc2)cc1]

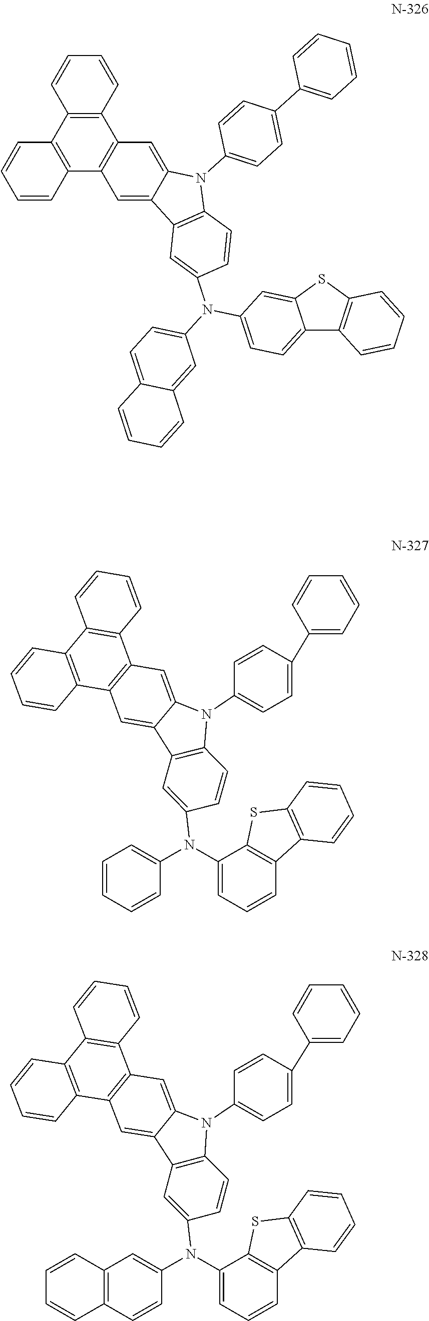 [SMILES: c1ccc(-c2ccc(-n3c4ccc(N(c5ccc6ccccc6c5)c5ccc6c(c5)sc5ccccc56)cc4c4cc5c6ccccc6c6ccccc6c5cc43)cc2)cc1 ; c1ccc(-c2ccc(-n3c4ccc(N(c5ccccc5)c5cccc6c5sc5ccccc56)cc4c4cc5c6ccccc6c6ccccc6c5cc43)cc2)cc1 ; c1ccc(-c2ccc(-n3c4ccc(N(c5ccc6ccccc6c5)c5cccc6c5sc5ccccc56)cc4c4cc5c6ccccc6c6ccccc6c5cc43)cc2)cc1]

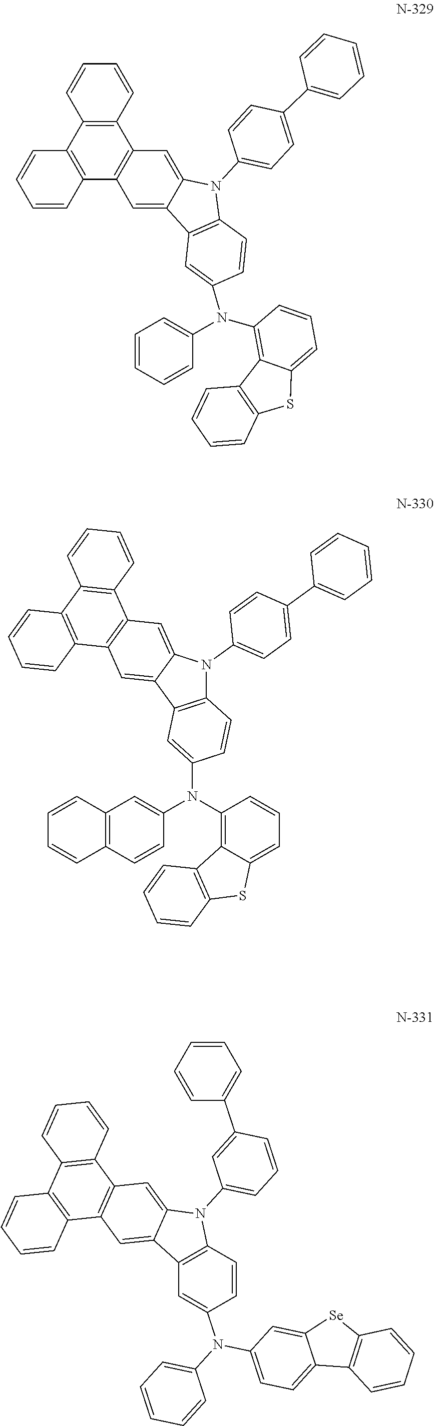

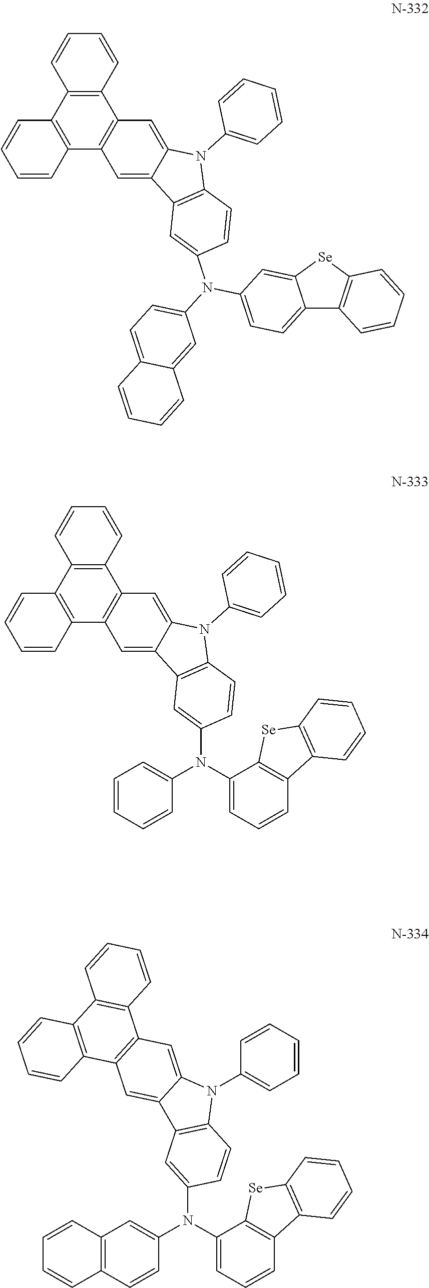

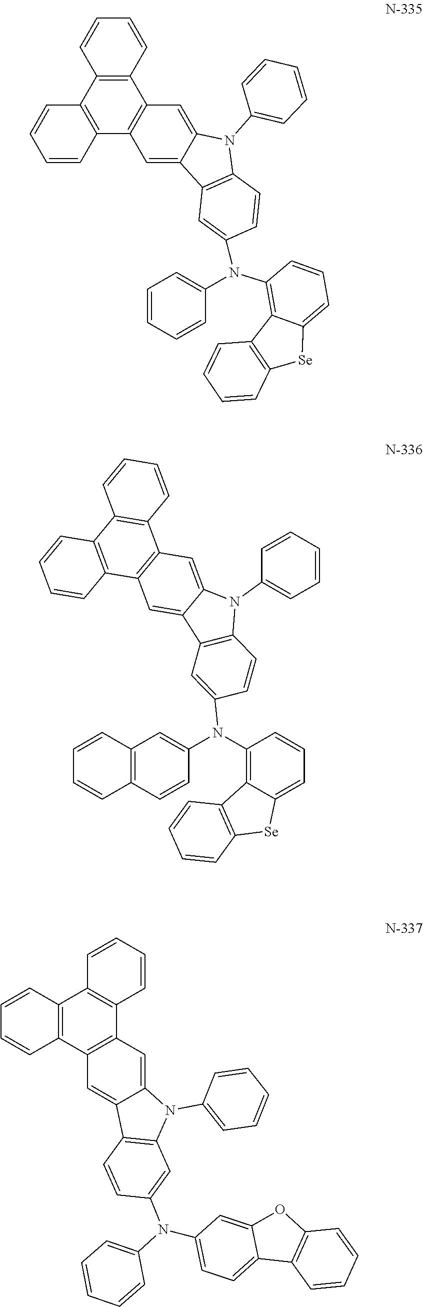 [SMILES: c1ccc(N(c2ccc3c(c2)c2cc4c5ccccc5c5ccccc5c4cc2n3-c2ccccc2)c2cccc3[se]c4ccccc4c23)cc1 ; c1ccc(-n2c3ccc(N(c4ccc5ccccc5c4)c4cccc5[se]c6ccccc6c45)cc3c3cc4c5ccccc5c5ccccc5c4cc32)cc1 ; c1ccc(N(c2ccc3c(c2)oc2ccccc23)c2ccc3c4cc5c6ccccc6c6ccccc6c5cc4n(-c4ccccc4)c3c2)cc1]

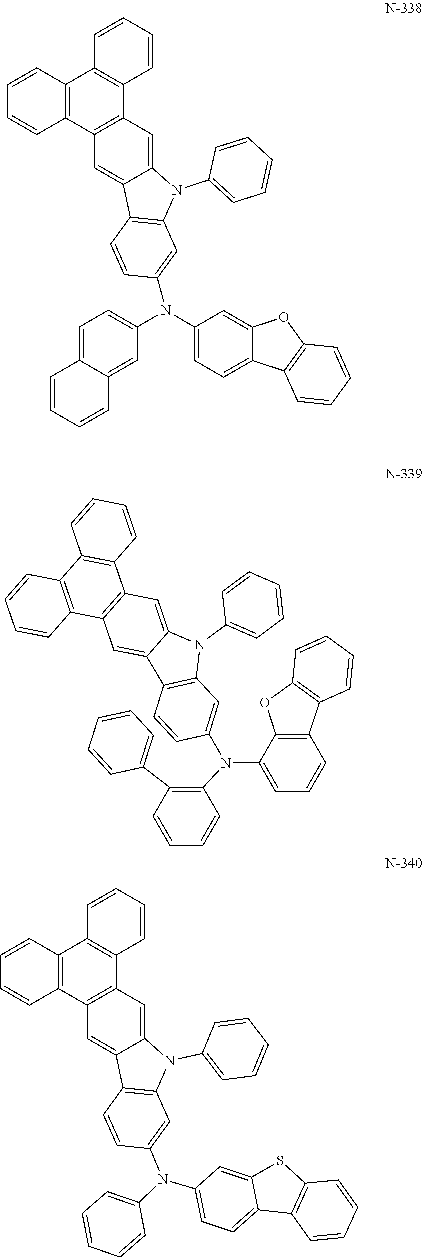 [SMILES: c1ccc(-n2c3cc(N(c4ccc5ccccc5c4)c4ccc5c(c4)oc4ccccc45)ccc3c3cc4c5ccccc5c5ccccc5c4cc32)cc1 ; c1ccc(-c2ccccc2N(c2ccc3c4cc5c6ccccc6c6ccccc6c5cc4n(-c4ccccc4)c3c2)c2cccc3c2oc2ccccc23)cc1 ; c1ccc(N(c2ccc3c(c2)sc2ccccc23)c2ccc3c4cc5c6ccccc6c6ccccc6c5cc4n(-c4ccccc4)c3c2)cc1]

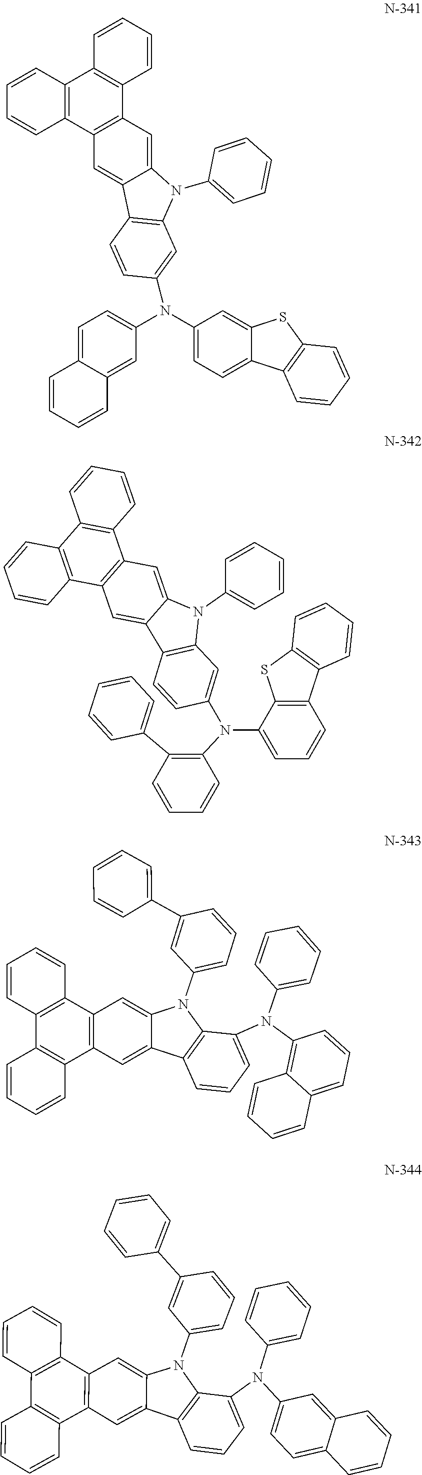

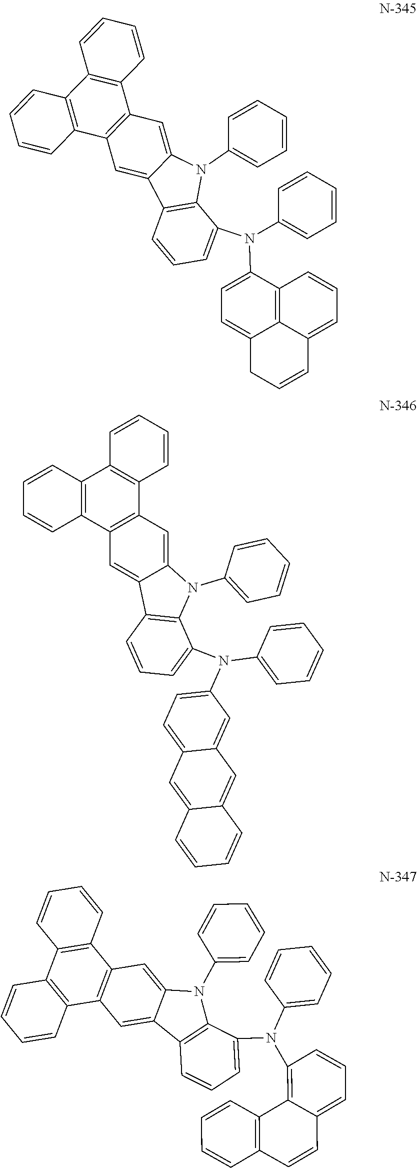

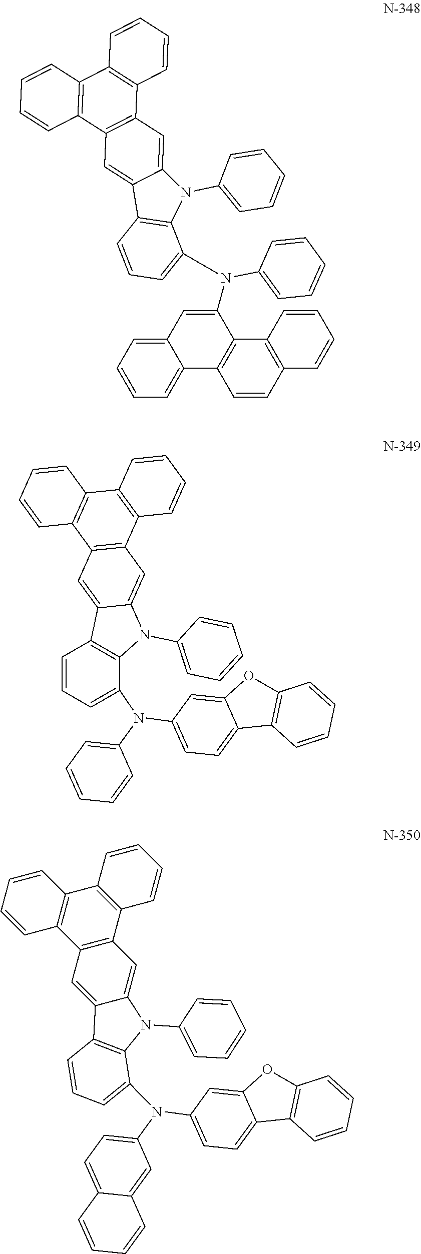 [SMILES: c1ccc(N(c2cc3ccccc3c3ccc4ccccc4c23)c2cccc3c4cc5c6ccccc6c6ccccc6c5cc4n(-c4ccccc4)c23)cc1 ; c1ccc(N(c2ccc3c(c2)oc2ccccc23)c2cccc3c4cc5c6ccccc6c6ccccc6c5cc4n(-c4ccccc4)c23)cc1 ; c1ccc(-n2c3cc4c5ccccc5c5ccccc5c4cc3c3cccc(N(c4ccc5ccccc5c4)c4ccc5c(c4)oc4ccccc45)c32)cc1]

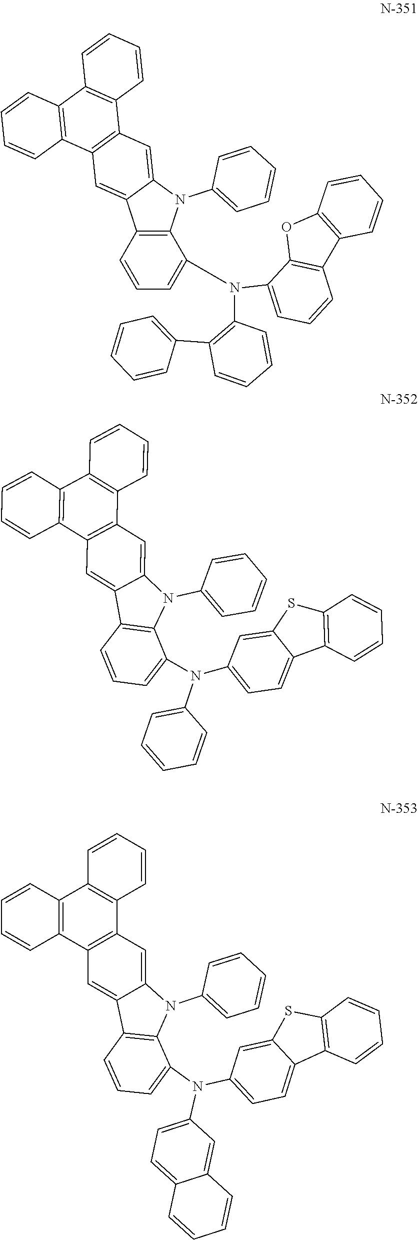

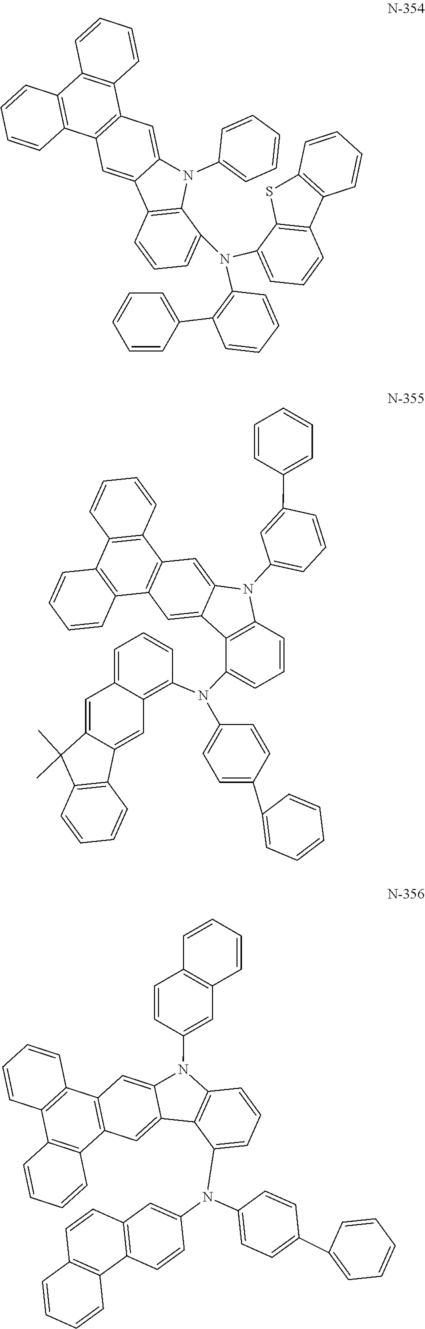 [SMILES: c1ccc(-c2ccccc2N(c2cccc3c2sc2ccccc23)c2cccc3c4cc5c6ccccc6c6ccccc6c5cc4n(-c4ccccc4)c23)cc1 ; CC1(C)c2ccccc2-c2cc3c(N(c4ccc(-c5ccccc5)cc4)c4cccc5c4c4cc6c7ccccc7c7ccccc7c6cc4n5-c4cccc(-c5ccccc5)c4)cccc3cc21 ; c1ccc(-c2ccc(N(c3ccc4c(ccc5ccccc54)c3)c3cccc4c3c3cc5c6ccccc6c6ccccc6c5cc3n4-c3ccc4ccccc4c3)cc2)cc1]

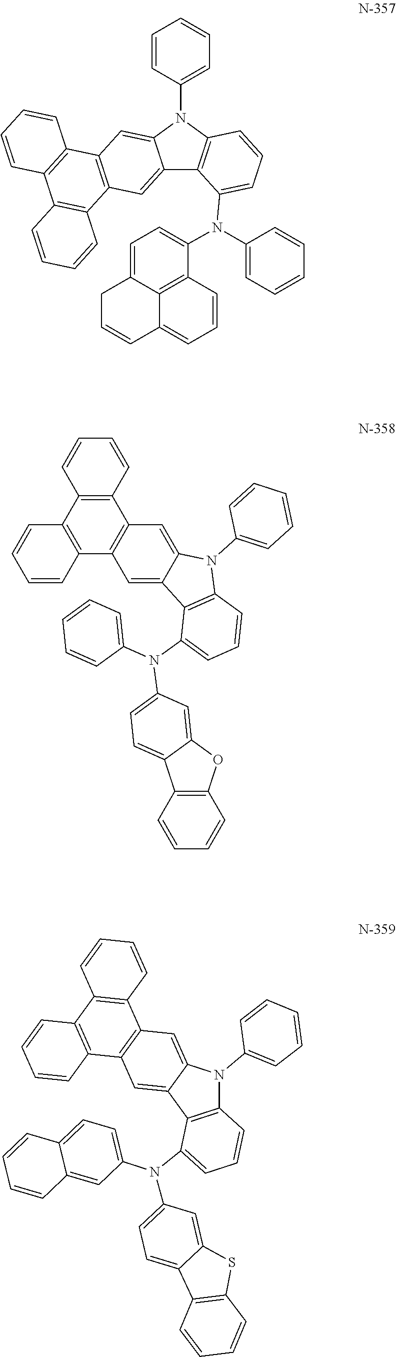 [SMILES: C1=Cc2cccc3c(N(c4ccccc4)c4cccc5c4c4cc6c7ccccc7c7ccccc7c6cc4n5-c4ccccc4)ccc(c23)C1 ; c1ccc(N(c2ccc3c(c2)oc2ccccc23)c2cccc3c2c2cc4c5ccccc5c5ccccc5c4cc2n3-c2ccccc2)cc1 ; c1ccc(-n2c3cc4c5ccccc5c5ccccc5c4cc3c3c(N(c4ccc5ccccc5c4)c4ccc5c(c4)sc4ccccc45)cccc32)cc1]

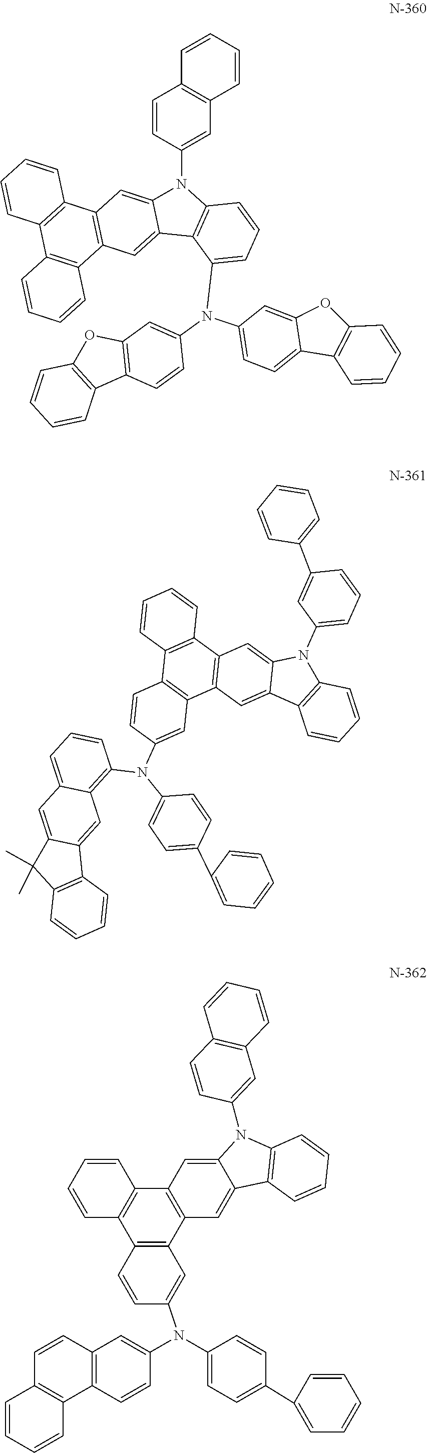 [SMILES: c1ccc2cc(-n3c4cc5c6ccccc6c6ccccc6c5cc4c4c(N(c5ccc6c(c5)oc5ccccc56)c5ccc6c(c5)oc5ccccc56)cccc43)ccc2c1 ; CC1(C)c2ccccc2-c2cc3c(N(c4ccc(-c5ccccc5)cc4)c4ccc5c6ccccc6c6cc7c(cc6c5c4)c4ccccc4n7-c4cccc(-c5ccccc5)c4)cccc3cc21 ; c1ccc(-c2ccc(N(c3ccc4c(ccc5ccccc54)c3)c3ccc4c5ccccc5c5cc6c(cc5c4c3)c3ccccc3n6-c3ccc4ccccc4c3)cc2)cc1]

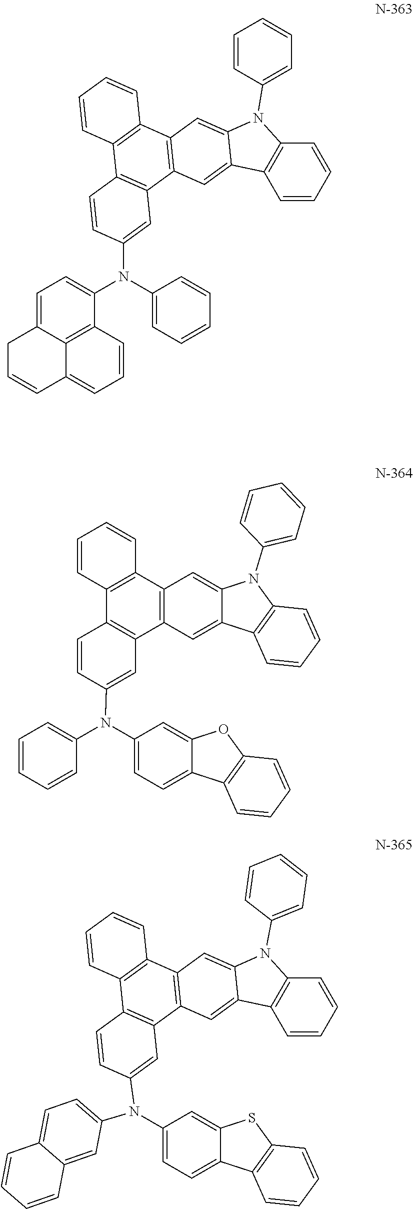

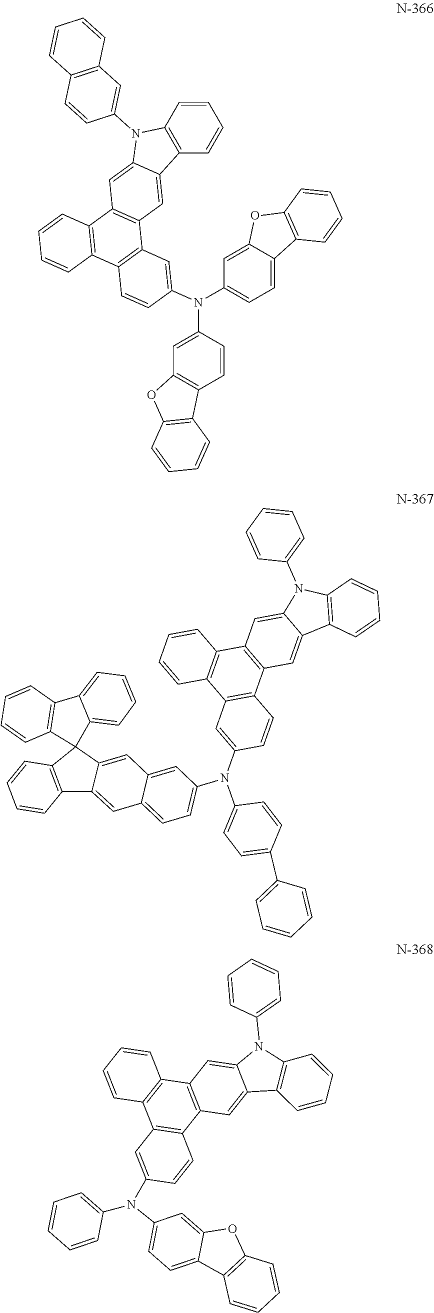

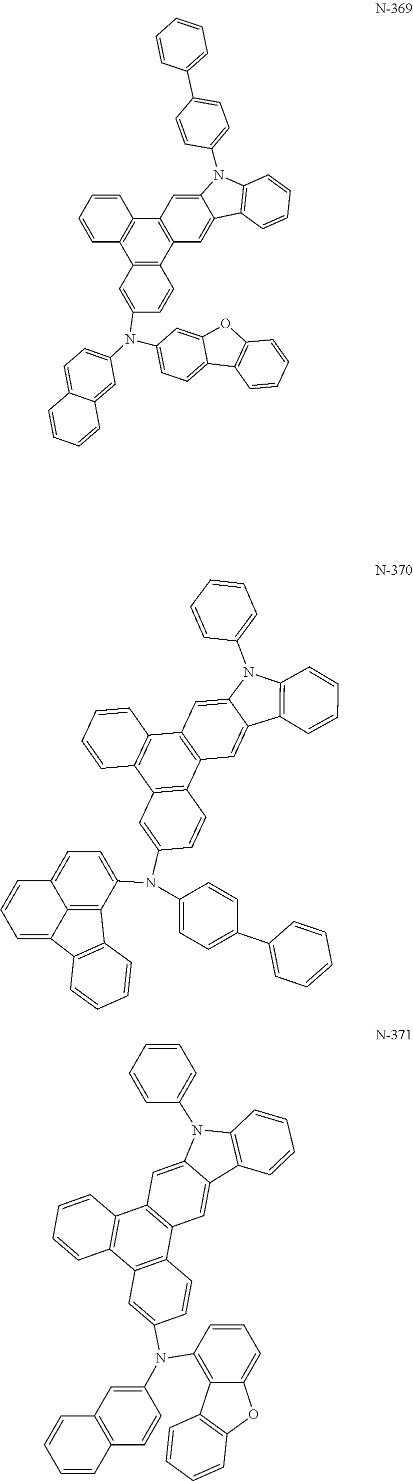 [SMILES: c1ccc(-c2ccc(-n3c4ccccc4c4cc5c6ccc(N(c7ccc8ccccc8c7)c7ccc8c(c7)oc7ccccc78)cc6c6ccccc6c5cc43)cc2)cc1 ; c1ccc(-c2ccc(N(c3ccc4c(c3)c3ccccc3c3cc5c(cc43)c3ccccc3n5-c3ccccc3)c3ccc4cccc5c4c3-c3ccccc3-5)cc2)cc1 ; c1ccc(-n2c3ccccc3c3cc4c5ccc(N(c6ccc7ccccc7c6)c6cccc7oc8ccccc8c67)cc5c5ccccc5c4cc32)cc1]

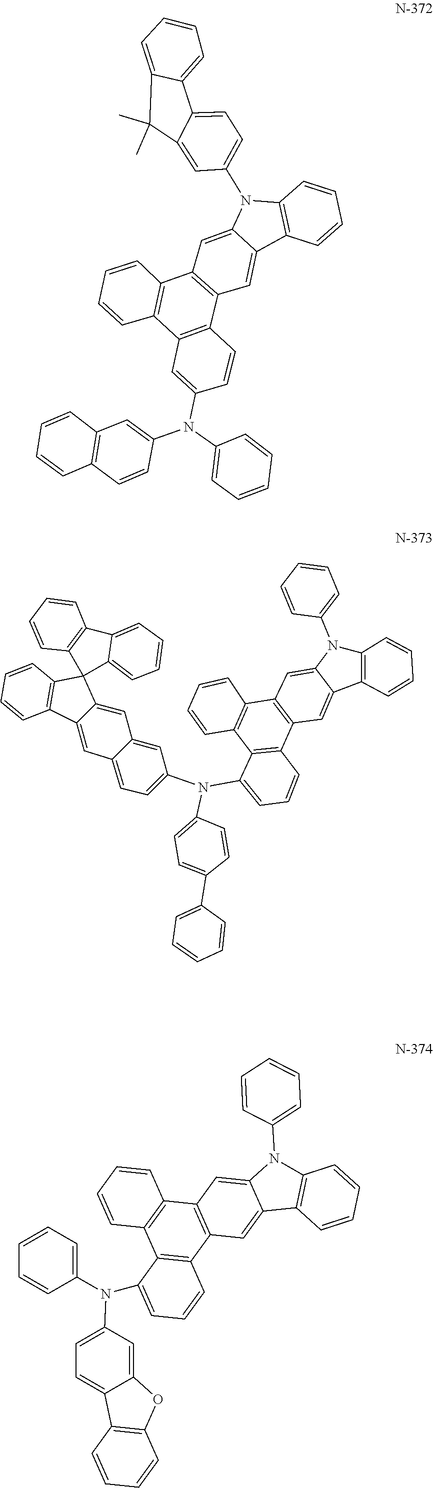 [SMILES: CC1(C)c2ccccc2-c2ccc(-n3c4ccccc4c4cc5c6ccc(N(c7ccccc7)c7ccc8ccccc8c7)cc6c6ccccc6c5cc43)cc21 ; c1ccc(-c2ccc(N(c3ccc4cc5c(cc4c3)C3(c4ccccc4-c4ccccc43)c3ccccc3-5)c3cccc4c5cc6c7ccccc7n(-c7ccccc7)c6cc5c5ccccc5c34)cc2)cc1 ; c1ccc(N(c2ccc3c(c2)oc2ccccc23)c2cccc3c4cc5c6ccccc6n(-c6ccccc6)c5cc4c4ccccc4c23)cc1]

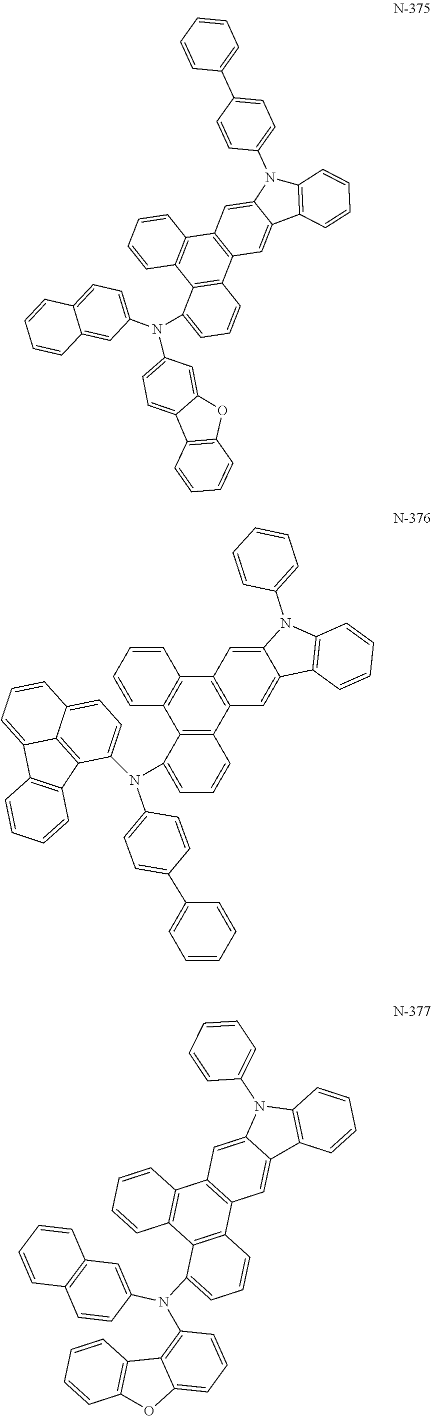 [SMILES: c1ccc(-c2ccc(-n3c4ccccc4c4cc5c(cc43)c3ccccc3c3c(N(c4ccc6ccccc6c4)c4ccc6c(c4)oc4ccccc46)cccc53)cc2)cc1 ; c1ccc(-c2ccc(N(c3ccc4cccc5c4c3-c3ccccc3-5)c3cccc4c5cc6c7ccccc7n(-c7ccccc7)c6cc5c5ccccc5c34)cc2)cc1 ; c1ccc(-n2c3ccccc3c3cc4c(cc32)c2ccccc2c2c(N(c3ccc5ccccc5c3)c3cccc5oc6ccccc6c35)cccc42)cc1]

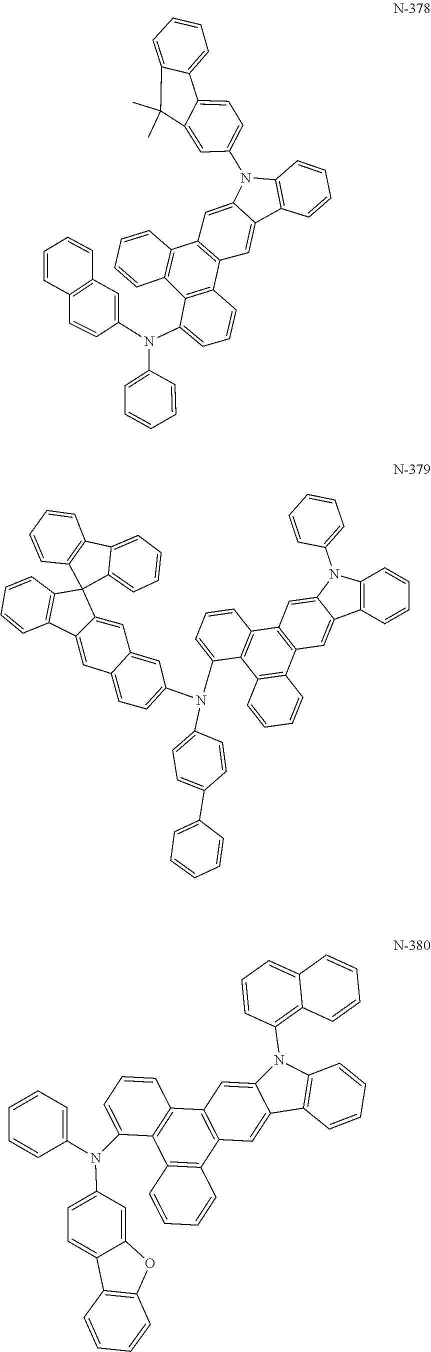 [SMILES: CC1(C)c2ccccc2-c2ccc(-n3c4ccccc4c4cc5c(cc43)c3ccccc3c3c(N(c4ccccc4)c4ccc6ccccc6c4)cccc53)cc21 ; c1ccc(-c2ccc(N(c3ccc4cc5c(cc4c3)C3(c4ccccc4-c4ccccc43)c3ccccc3-5)c3cccc4c5cc6c(cc5c5ccccc5c34)c3ccccc3n6-c3ccccc3)cc2)cc1 ; c1ccc(N(c2ccc3c(c2)oc2ccccc23)c2cccc3c4cc5c(cc4c4ccccc4c23)c2ccccc2n5-c2cccc3ccccc23)cc1]

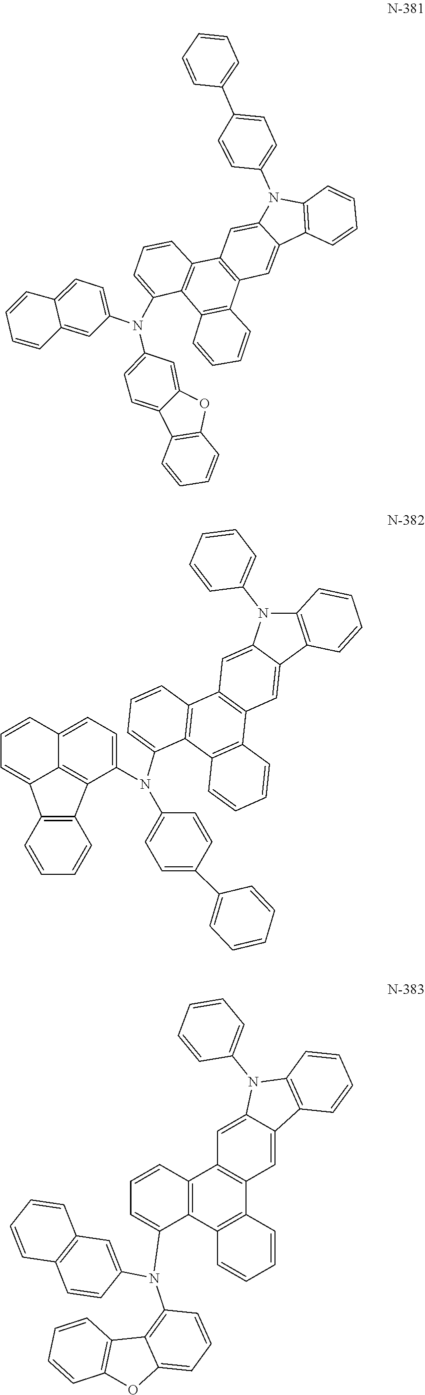 [SMILES: c1ccc(-c2ccc(-n3c4ccccc4c4cc5c6ccccc6c6c(N(c7ccc8ccccc8c7)c7ccc8c(c7)oc7ccccc78)cccc6c5cc43)cc2)cc1 ; c1ccc(-c2ccc(N(c3ccc4cccc5c4c3-c3ccccc3-5)c3cccc4c5cc6c(cc5c5ccccc5c34)c3ccccc3n6-c3ccccc3)cc2)cc1 ; c1ccc(-n2c3ccccc3c3cc4c5ccccc5c5c(N(c6ccc7ccccc7c6)c6cccc7oc8ccccc8c67)cccc5c4cc32)cc1]

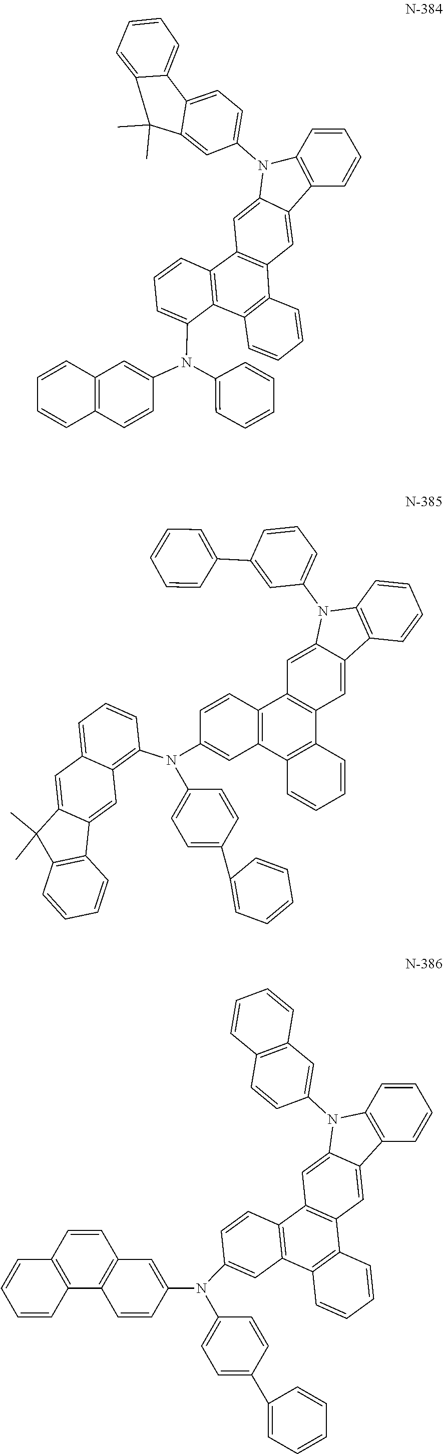 [SMILES: CC1(C)c2ccccc2-c2ccc(-n3c4ccccc4c4cc5c6ccccc6c6c(N(c7ccccc7)c7ccc8ccccc8c7)cccc6c5cc43)cc21 ; CC1(C)c2ccccc2-c2cc3c(N(c4ccc(-c5ccccc5)cc4)c4ccc5c(c4)c4ccccc4c4cc6c7ccccc7n(-c7cccc(-c8ccccc8)c7)c6cc54)cccc3cc21 ; c1ccc(-c2ccc(N(c3ccc4c(ccc5ccccc54)c3)c3ccc4c(c3)c3ccccc3c3cc5c6ccccc6n(-c6ccc7ccccc7c6)c5cc43)cc2)cc1]

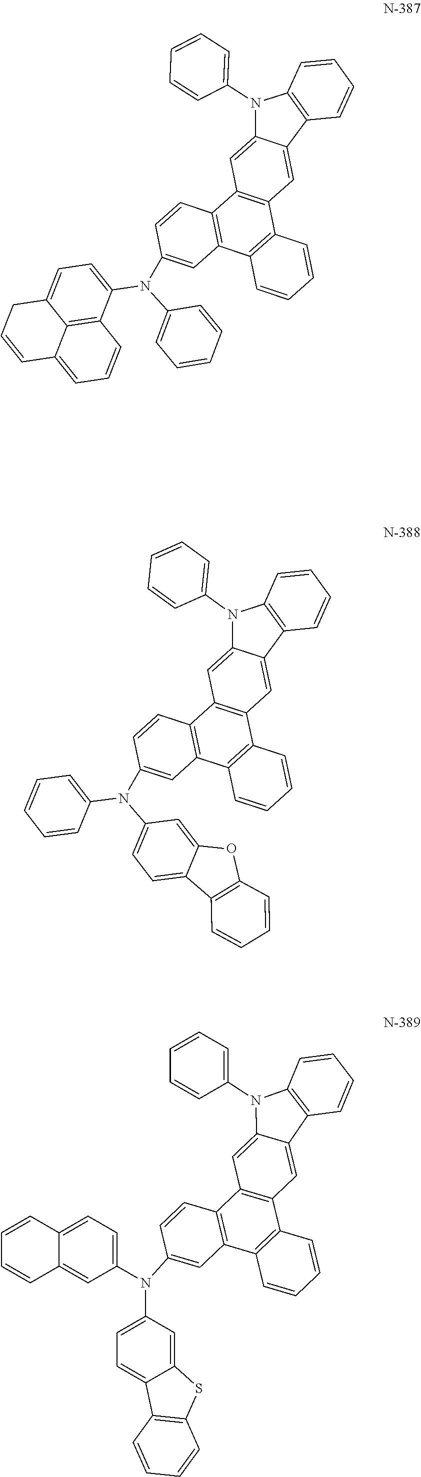 [SMILES: C1=Cc2cccc3c(N(c4ccccc4)c4ccc5c(c4)c4ccccc4c4cc6c7ccccc7n(-c7ccccc7)c6cc54)ccc(c23)C1 ; c1ccc(N(c2ccc3c(c2)oc2ccccc23)c2ccc3c(c2)c2ccccc2c2cc4c5ccccc5n(-c5ccccc5)c4cc32)cc1 ; c1ccc(-n2c3ccccc3c3cc4c5ccccc5c5cc(N(c6ccc7ccccc7c6)c6ccc7c(c6)sc6ccccc67)ccc5c4cc32)cc1]

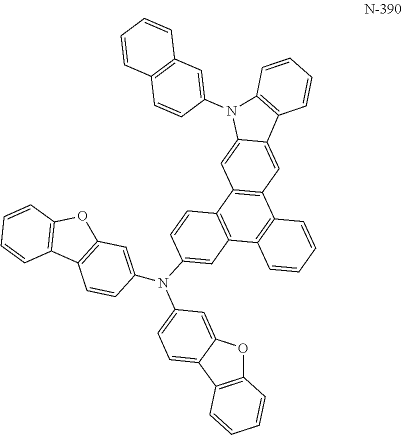 [SMILES: c1ccc2cc(-n3c4ccccc4c4cc5c6ccccc6c6cc(N(c7ccc8c(c7)oc7ccccc78)c7ccc8c(c7)oc7ccccc78)ccc6c5cc43)ccc2c1]

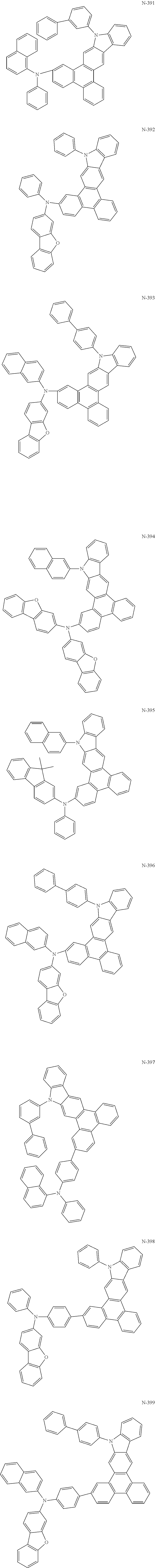

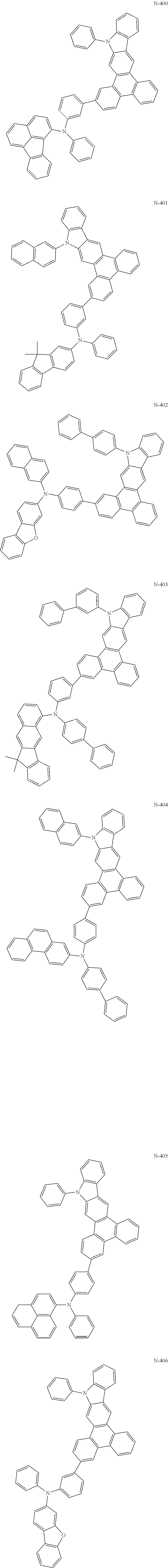

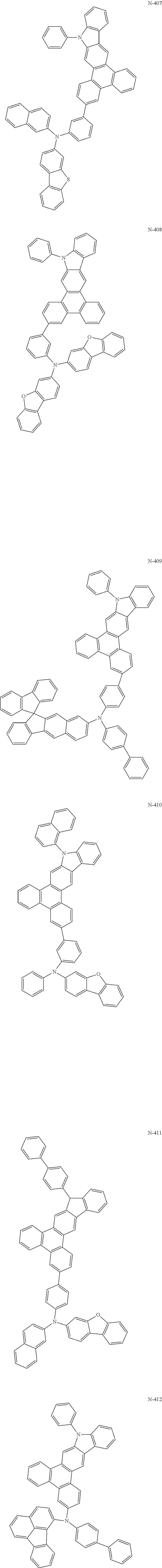

[chemical structure]

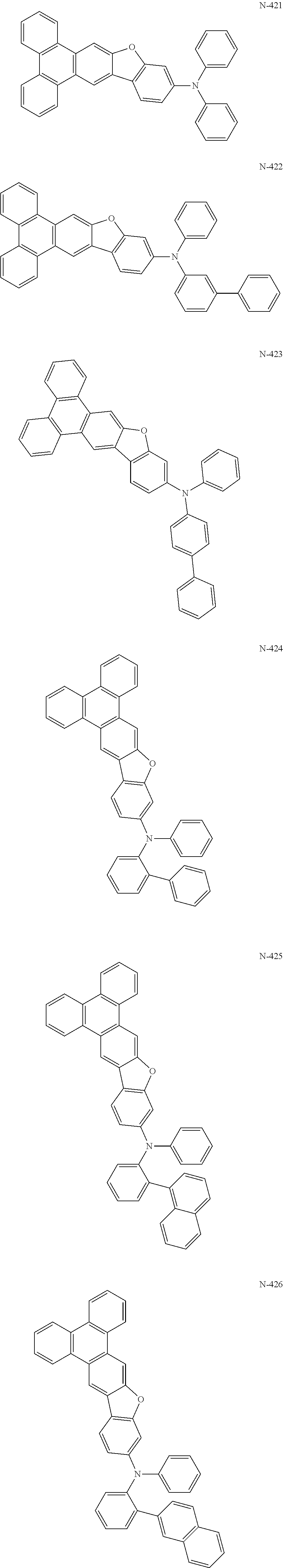 [SMILES: c1ccc(N(c2ccccc2)c2ccc3c(c2)oc2cc4c5ccccc5c5ccccc5c4cc23)cc1 ; c1ccc(-c2cccc(N(c3ccccc3)c3ccc4c(c3)oc3cc5c6ccccc6c6ccccc6c5cc34)c2)cc1 ; c1ccc(-c2ccc(N(c3ccccc3)c3ccc4c(c3)oc3cc5c6ccccc6c6ccccc6c5cc34)cc2)cc1 ; c1ccc(-c2ccccc2N(c2ccccc2)c2ccc3c(c2)oc2cc4c5ccccc5c5ccccc5c4cc23)cc1 ; c1ccc(N(c2ccc3c(c2)oc2cc4c5ccccc5c5ccccc5c4cc23)c2ccccc2-c2cccc3ccccc23)cc1 ; c1ccc(N(c2ccc3c(c2)oc2cc4c5ccccc5c5ccccc5c4cc23)c2ccccc2-c2ccc3ccccc3c2)cc1]

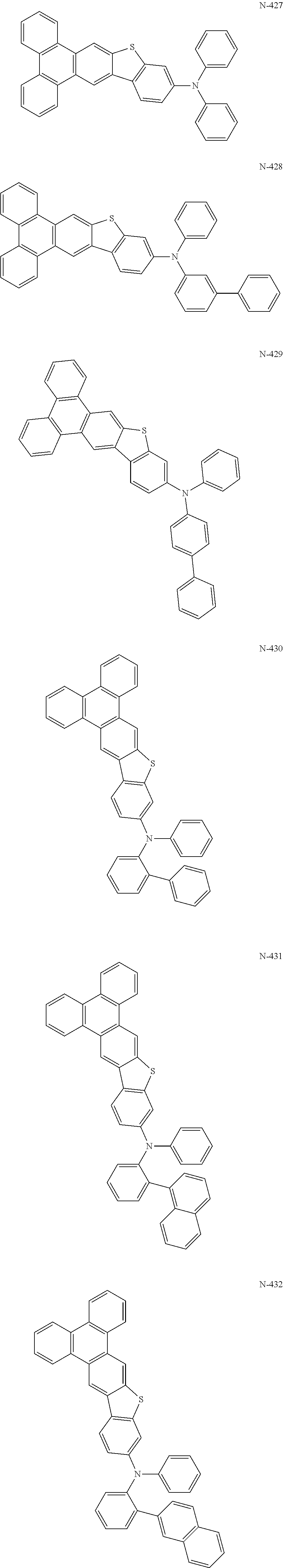 [SMILES: c1ccc(N(c2ccccc2)c2ccc3c(c2)sc2cc4c5ccccc5c5ccccc5c4cc23)cc1 ; c1ccc(-c2cccc(N(c3ccccc3)c3ccc4c(c3)sc3cc5c6ccccc6c6ccccc6c5cc34)c2)cc1 ; c1ccc(-c2ccc(N(c3ccccc3)c3ccc4c(c3)sc3cc5c6ccccc6c6ccccc6c5cc34)cc2)cc1 ; c1ccc(-c2ccccc2N(c2ccccc2)c2ccc3c(c2)sc2cc4c5ccccc5c5ccccc5c4cc23)cc1 ; c1ccc(N(c2ccc3c(c2)sc2cc4c5ccccc5c5ccccc5c4cc23)c2ccccc2-c2cccc3ccccc23)cc1 ; c1ccc(N(c2ccc3c(c2)sc2cc4c5ccccc5c5ccccc5c4cc23)c2ccccc2-c2ccc3ccccc3c2)cc1]

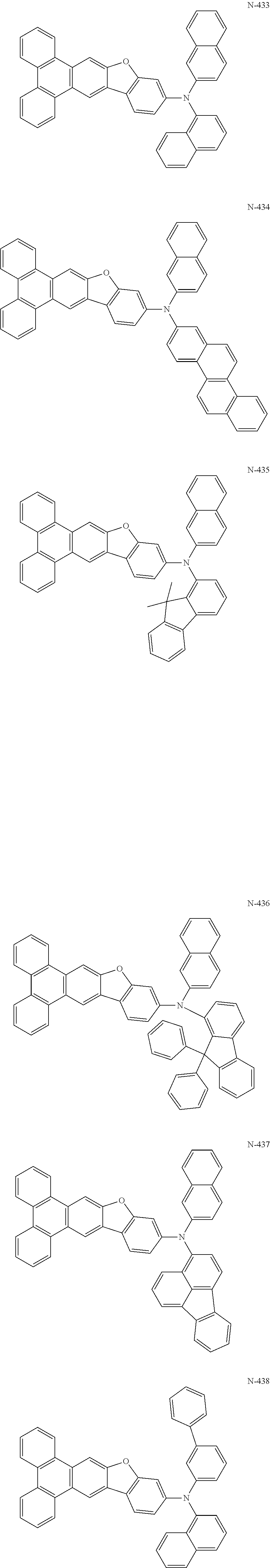

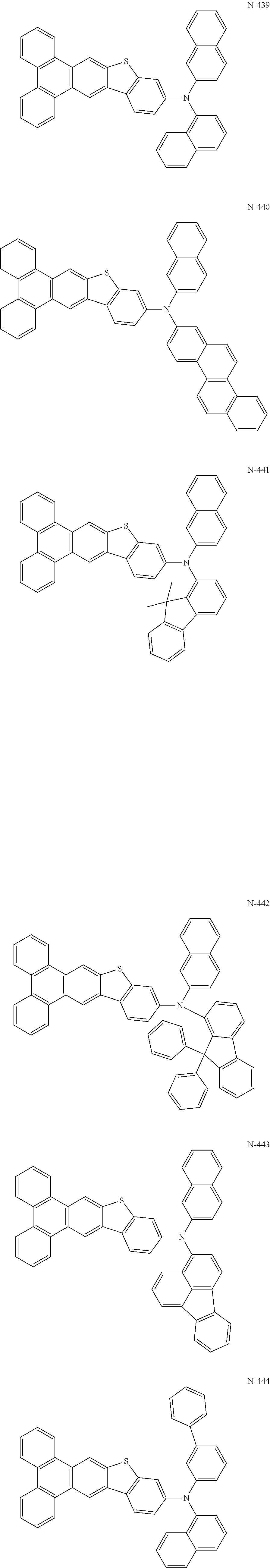

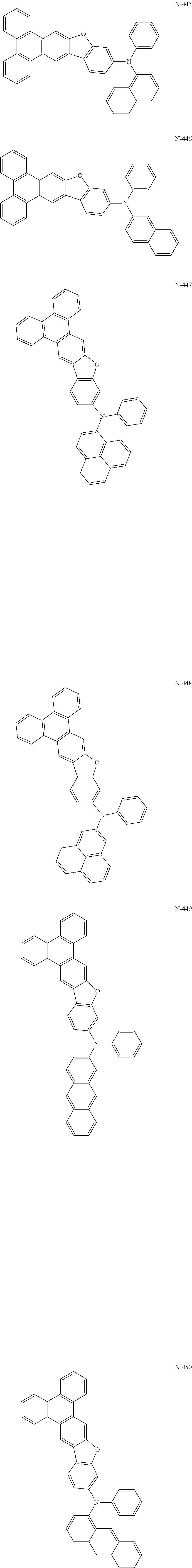 [SMILES: c1ccc(N(c2ccc3c(c2)oc2cc4c5ccccc5c5ccccc5c4cc23)c2cccc3ccccc23)cc1 ; c1ccc(N(c2ccc3ccccc3c2)c2ccc3c(c2)oc2cc4c5ccccc5c5ccccc5c4cc23)cc1 ; C1=Cc2cccc3c(N(c4ccccc4)c4ccc5c(c4)oc4cc6c7ccccc7c7ccccc7c6cc45)ccc(c23)C1 ; C1=Cc2cccc3cc(N(c4ccccc4)c4ccc5c(c4)oc4cc6c7ccccc7c7ccccc7c6cc45)cc(c23)C1 ; c1ccc(N(c2ccc3cc4ccccc4cc3c2)c2ccc3c(c2)oc2cc4c5ccccc5c5ccccc5c4cc23)cc1 ; c1ccc(N(c2ccc3c(c2)oc2cc4c5ccccc5c5ccccc5c4cc23)c2cccc3cc4ccccc4cc23)cc1]

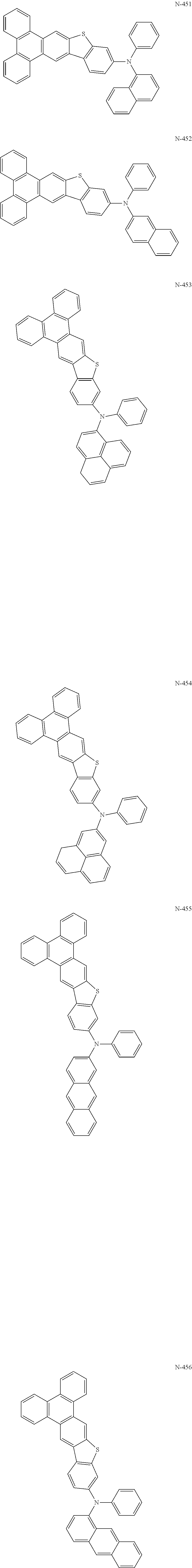 [SMILES: c1ccc(N(c2ccc3c(c2)sc2cc4c5ccccc5c5ccccc5c4cc23)c2cccc3ccccc23)cc1 ; c1ccc(N(c2ccc3ccccc3c2)c2ccc3c(c2)sc2cc4c5ccccc5c5ccccc5c4cc23)cc1 ; C1=Cc2cccc3c(N(c4ccccc4)c4ccc5c(c4)sc4cc6c7ccccc7c7ccccc7c6cc45)ccc(c23)C1 ; C1=Cc2cccc3cc(N(c4ccccc4)c4ccc5c(c4)sc4cc6c7ccccc7c7ccccc7c6cc45)cc(c23)C1 ; c1ccc(N(c2ccc3cc4ccccc4cc3c2)c2ccc3c(c2)sc2cc4c5ccccc5c5ccccc5c4cc23)cc1 ; c1ccc(N(c2ccc3c(c2)sc2cc4c5ccccc5c5ccccc5c4cc23)c2cccc3cc4ccccc4cc23)cc1]

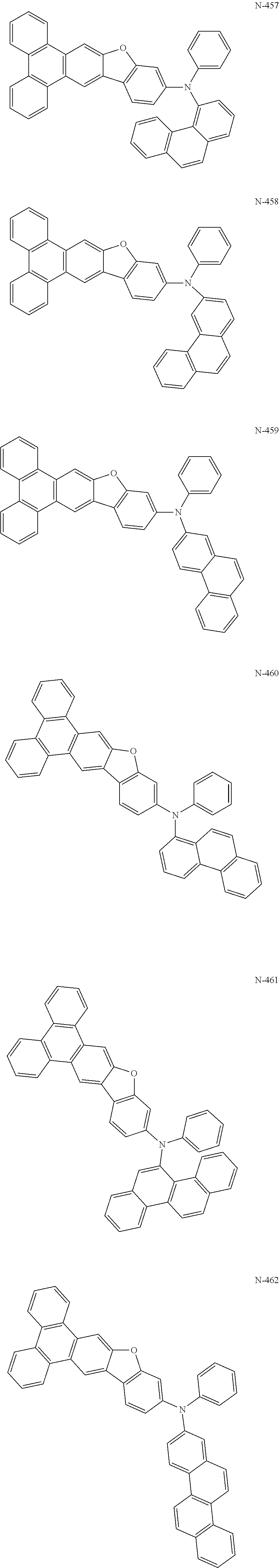 [SMILES: c1ccc(N(c2ccc3c(c2)oc2cc4c5ccccc5c5ccccc5c4cc23)c2cccc3ccc4ccccc4c23)cc1 ; c1ccc(N(c2ccc3c(c2)oc2cc4c5ccccc5c5ccccc5c4cc23)c2ccc3ccc4ccccc4c3c2)cc1 ; c1ccc(N(c2ccc3c(ccc4ccccc43)c2)c2ccc3c(c2)oc2cc4c5ccccc5c5ccccc5c4cc23)cc1 ; c1ccc(N(c2ccc3c(c2)oc2cc4c5ccccc5c5ccccc5c4cc23)c2cccc3c2ccc2ccccc23)cc1 ; c1ccc(N(c2ccc3c(c2)oc2cc4c5ccccc5c5ccccc5c4cc23)c2cc3ccccc3c3ccc4ccccc4c23)cc1 ; c1ccc(N(c2ccc3c(ccc4c5ccccc5ccc34)c2)c2ccc3c(c2)oc2cc4c5ccccc5c5ccccc5c4cc23)cc1]

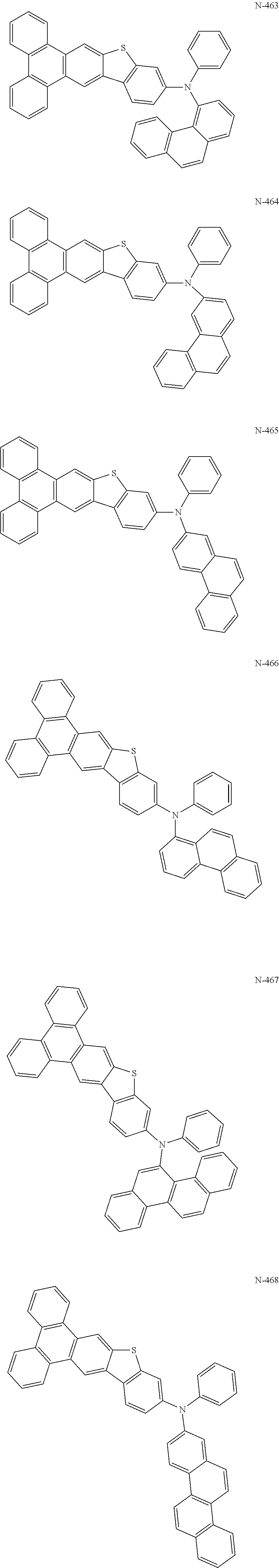 [SMILES: c1ccc(N(c2ccc3c(c2)sc2cc4c5ccccc5c5ccccc5c4cc23)c2cccc3ccc4ccccc4c23)cc1 ; c1ccc(N(c2ccc3c(c2)sc2cc4c5ccccc5c5ccccc5c4cc23)c2ccc3ccc4ccccc4c3c2)cc1 ; c1ccc(N(c2ccc3c(ccc4ccccc43)c2)c2ccc3c(c2)sc2cc4c5ccccc5c5ccccc5c4cc23)cc1 ; c1ccc(N(c2ccc3c(c2)sc2cc4c5ccccc5c5ccccc5c4cc23)c2cccc3c2ccc2ccccc23)cc1 ; c1ccc(N(c2ccc3c(c2)sc2cc4c5ccccc5c5ccccc5c4cc23)c2cc3ccccc3c3ccc4ccccc4c23)cc1 ; c1ccc(N(c2ccc3c(ccc4c5ccccc5ccc34)c2)c2ccc3c(c2)sc2cc4c5ccccc5c5ccccc5c4cc23)cc1]

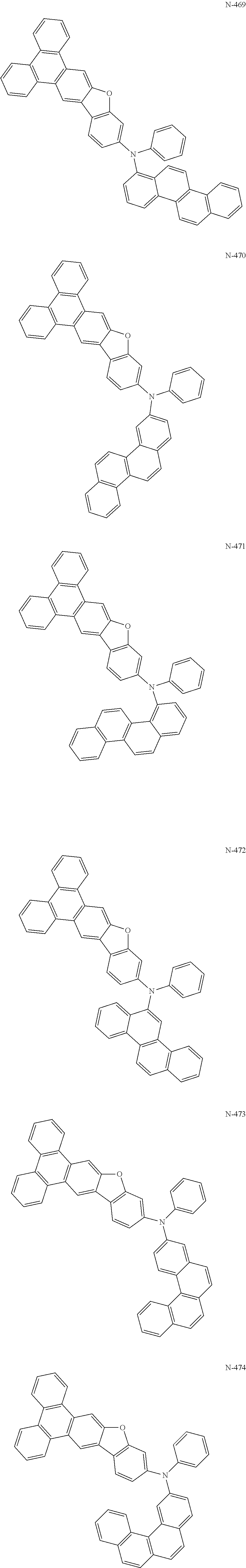 [SMILES: c1ccc(N(c2ccc3c(c2)oc2cc4c5ccccc5c5ccccc5c4cc23)c2cccc3c2ccc2c4ccccc4ccc32)cc1 ; c1ccc(N(c2ccc3c(c2)oc2cc4c5ccccc5c5ccccc5c4cc23)c2ccc3ccc4c5ccccc5ccc4c3c2)cc1 ; c1ccc(N(c2ccc3c(c2)oc2cc4c5ccccc5c5ccccc5c4cc23)c2cccc3ccc4c5ccccc5ccc4c23)cc1 ; c1ccc(N(c2ccc3c(c2)oc2cc4c5ccccc5c5ccccc5c4cc23)c2cc3c4ccccc4ccc3c3ccccc23)cc1 ; c1ccc(N(c2ccc3c(ccc4ccc5ccccc5c43)c2)c2ccc3c(c2)oc2cc4c5ccccc5c5ccccc5c4cc23)cc1 ; c1ccc(N(c2ccc3c(c2)oc2cc4c5ccccc5c5ccccc5c4cc23)c2ccc3ccc4ccc5ccccc5c4c3c2)cc1]

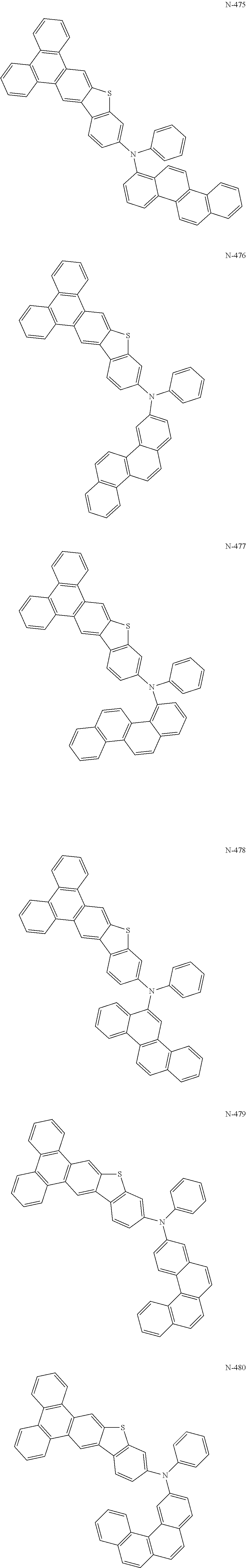 [SMILES: c1ccc(N(c2ccc3c(c2)sc2cc4c5ccccc5c5ccccc5c4cc23)c2cccc3c2ccc2c4ccccc4ccc32)cc1 ; c1ccc(N(c2ccc3c(c2)sc2cc4c5ccccc5c5ccccc5c4cc23)c2ccc3ccc4c5ccccc5ccc4c3c2)cc1 ; c1ccc(N(c2ccc3c(c2)sc2cc4c5ccccc5c5ccccc5c4cc23)c2cccc3ccc4c5ccccc5ccc4c23)cc1 ; c1ccc(N(c2ccc3c(c2)sc2cc4c5ccccc5c5ccccc5c4cc23)c2cc3c4ccccc4ccc3c3ccccc23)cc1 ; c1ccc(N(c2ccc3c(ccc4ccc5ccccc5c43)c2)c2ccc3c(c2)sc2cc4c5ccccc5c5ccccc5c4cc23)cc1 ; c1ccc(N(c2ccc3c(c2)sc2cc4c5ccccc5c5ccccc5c4cc23)c2ccc3ccc4ccc5ccccc5c4c3c2)cc1]

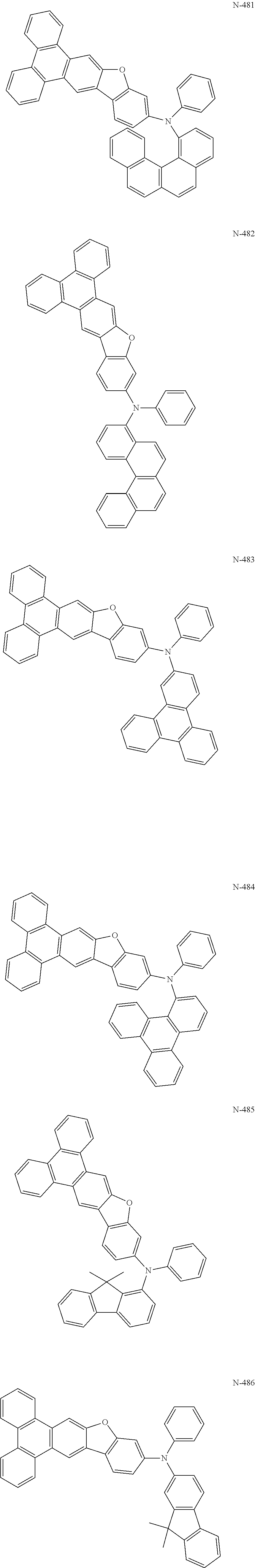 [SMILES: c1ccc(N(c2ccc3c(c2)oc2cc4c5ccccc5c5ccccc5c4cc23)c2cccc3ccc4ccc5ccccc5c4c23)cc1 ; c1ccc(N(c2ccc3c(c2)oc2cc4c5ccccc5c5ccccc5c4cc23)c2cccc3c2ccc2ccc4ccccc4c23)cc1 ; c1ccc(N(c2ccc3c(c2)oc2cc4c5ccccc5c5ccccc5c4cc23)c2ccc3c4ccccc4c4ccccc4c3c2)cc1 ; c1ccc(N(c2ccc3c(c2)oc2cc4c5ccccc5c5ccccc5c4cc23)c2cccc3c4ccccc4c4ccccc4c23)cc1 ; CC1(C)c2ccccc2-c2cccc(N(c3ccccc3)c3ccc4c(c3)oc3cc5c6ccccc6c6ccccc6c5cc34)c21 ; CC1(C)c2ccccc2-c2ccc(N(c3ccccc3)c3ccc4c(c3)oc3cc5c6ccccc6c6ccccc6c5cc34)cc21]

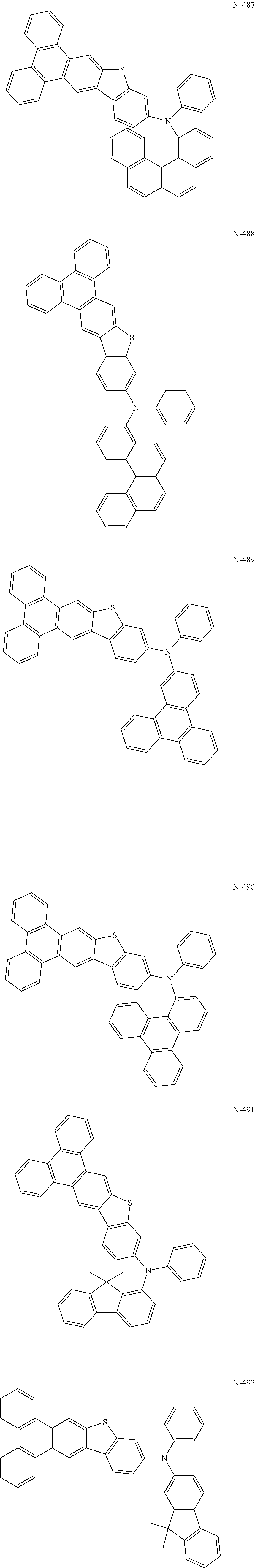 [SMILES: c1ccc(N(c2ccc3c(c2)sc2cc4c5ccccc5c5ccccc5c4cc23)c2cccc3ccc4ccc5ccccc5c4c23)cc1 ; c1ccc(N(c2ccc3c(c2)sc2cc4c5ccccc5c5ccccc5c4cc23)c2cccc3c2ccc2ccc4ccccc4c23)cc1 ; c1ccc(N(c2ccc3c(c2)sc2cc4c5ccccc5c5ccccc5c4cc23)c2ccc3c4ccccc4c4ccccc4c3c2)cc1 ; c1ccc(N(c2ccc3c(c2)sc2cc4c5ccccc5c5ccccc5c4cc23)c2cccc3c4ccccc4c4ccccc4c23)cc1 ; CC1(C)c2ccccc2-c2cccc(N(c3ccccc3)c3ccc4c(c3)sc3cc5c6ccccc6c6ccccc6c5cc34)c21 ; CC1(C)c2ccccc2-c2ccc(N(c3ccccc3)c3ccc4c(c3)sc3cc5c6ccccc6c6ccccc6c5cc34)cc21]

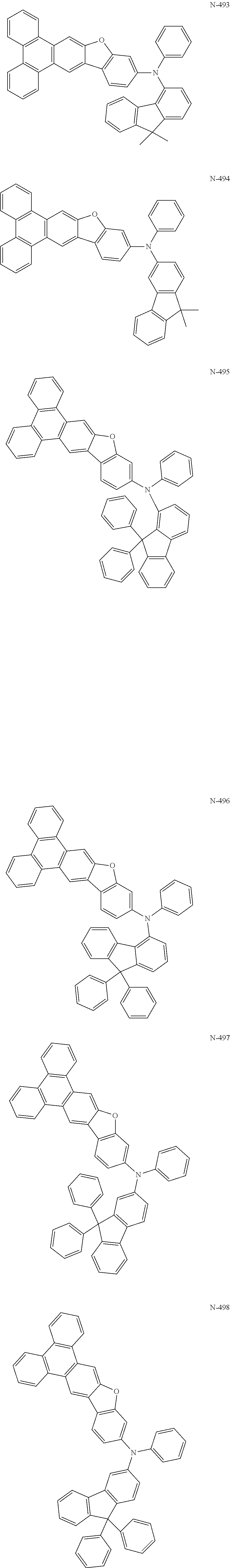

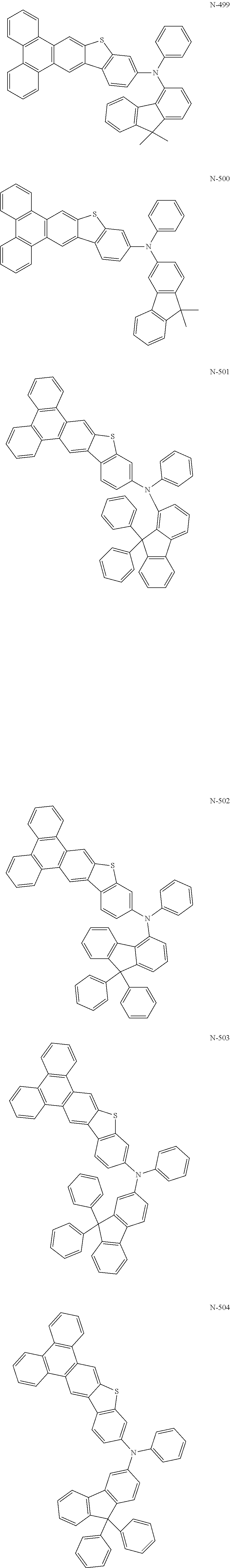

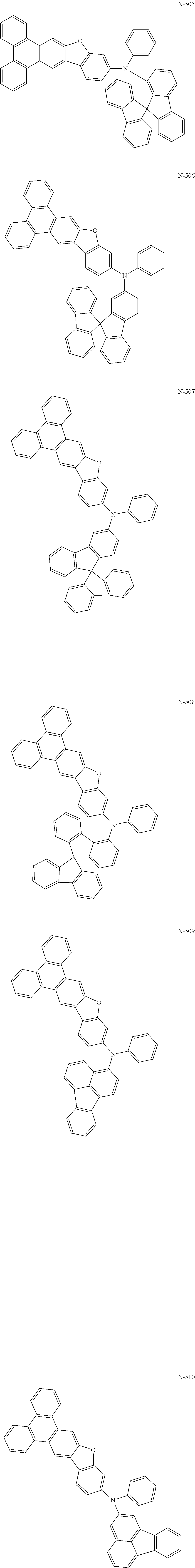

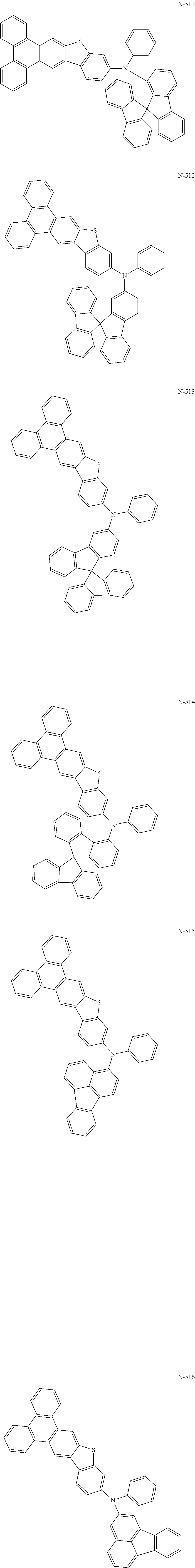

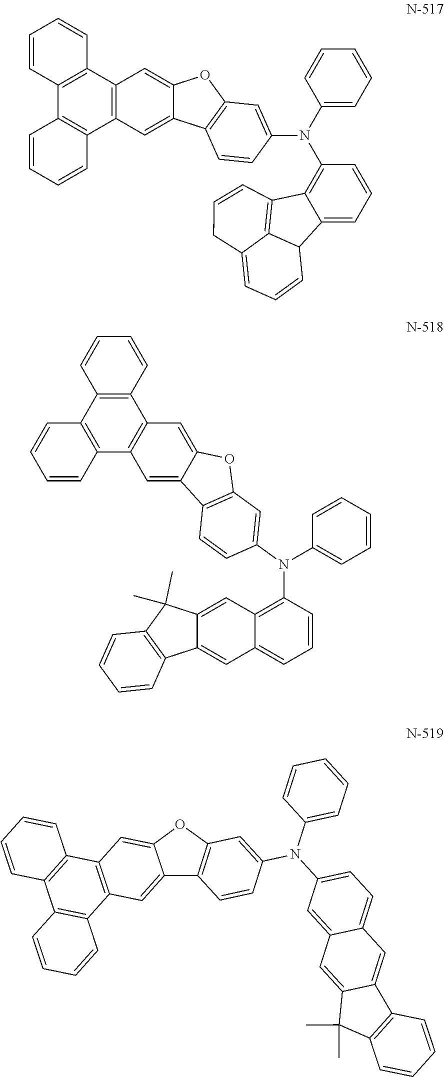 [SMILES: C1=CC2C3=C(C=CCC3=C1)c1c2cccc1N(c1ccccc1)c1ccc2c(c1)oc1cc3c4ccccc4c4ccccc4c3cc12 ; CC1(C)c2ccccc2-c2cc3cccc(N(c4ccccc4)c4ccc5c(c4)oc4cc6c7ccccc7c7ccccc7c6cc45)c3cc21 ; CC1(C)c2ccccc2-c2cc3ccc(N(c4ccccc4)c4ccc5c(c4)oc4cc6c7ccccc7c7ccccc7c6cc45)cc3cc21]

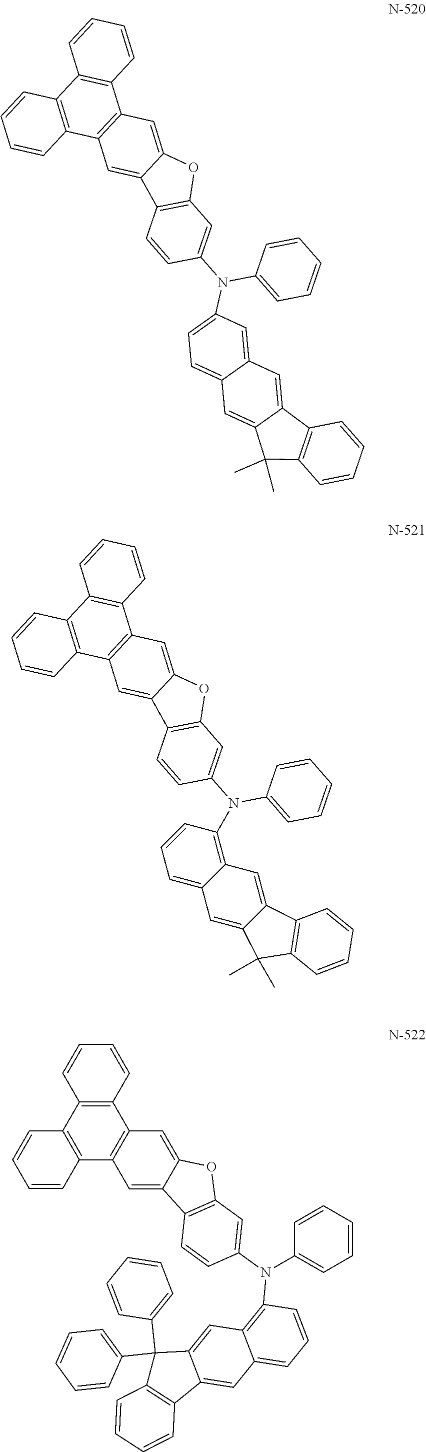 [SMILES: CC1(C)c2ccccc2-c2cc3cc(N(c4ccccc4)c4ccc5c(c4)oc4cc6c7ccccc7c7ccccc7c6cc45)ccc3cc21 ; CC1(C)c2ccccc2-c2cc3c(N(c4ccccc4)c4ccc5c(c4)oc4cc6c7ccccc7c7ccccc7c6cc45)cccc3cc21 ; c1ccc(N(c2ccc3c(c2)oc2cc4c5ccccc5c5ccccc5c4cc23)c2cccc3cc4c(cc23)C(c2ccccc2)(c2ccccc2)c2ccccc2-4)cc1]

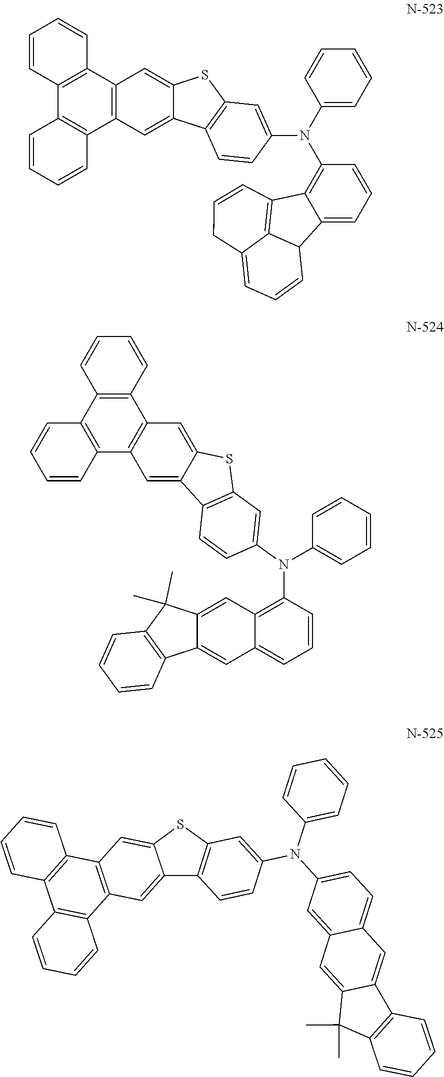 [SMILES: C1=CC2C3=C(C=CCC3=C1)c1c2cccc1N(c1ccccc1)c1ccc2c(c1)sc1cc3c4ccccc4c4ccccc4c3cc12 ; CC1(C)c2ccccc2-c2cc3cccc(N(c4ccccc4)c4ccc5c(c4)sc4cc6c7ccccc7c7ccccc7c6cc45)c3cc21 ; CC1(C)c2ccccc2-c2cc3ccc(N(c4ccccc4)c4ccc5c(c4)sc4cc6c7ccccc7c7ccccc7c6cc45)cc3cc21]

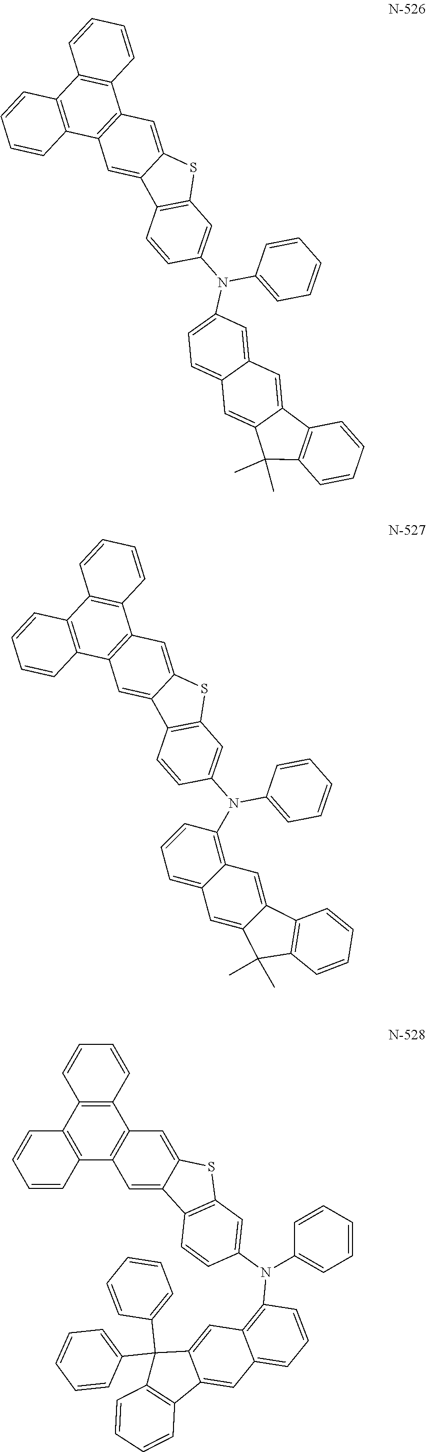 [SMILES: CC1(C)c2ccccc2-c2cc3cc(N(c4ccccc4)c4ccc5c(c4)sc4cc6c7ccccc7c7ccccc7c6cc45)ccc3cc21 ; CC1(C)c2ccccc2-c2cc3c(N(c4ccccc4)c4ccc5c(c4)sc4cc6c7ccccc7c7ccccc7c6cc45)cccc3cc21 ; c1ccc(N(c2ccc3c(c2)sc2cc4c5ccccc5c5ccccc5c4cc23)c2cccc3cc4c(cc23)C(c2ccccc2)(c2ccccc2)c2ccccc2-4)cc1]

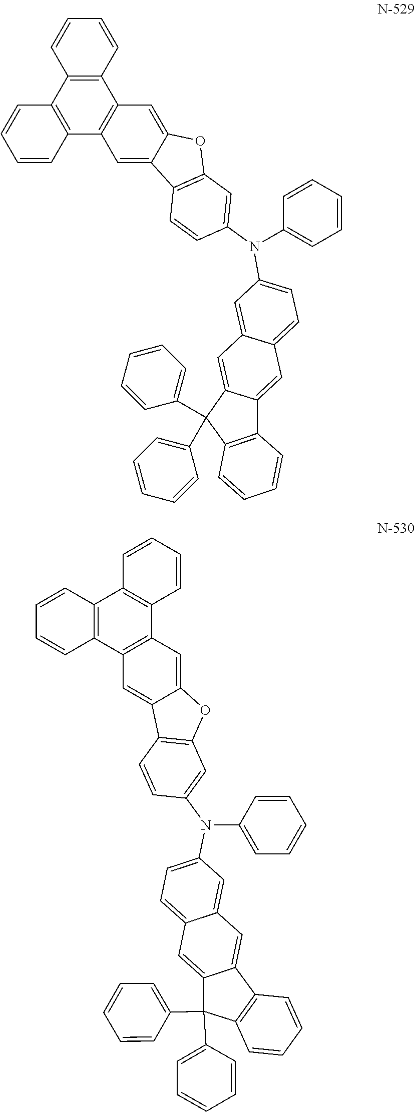

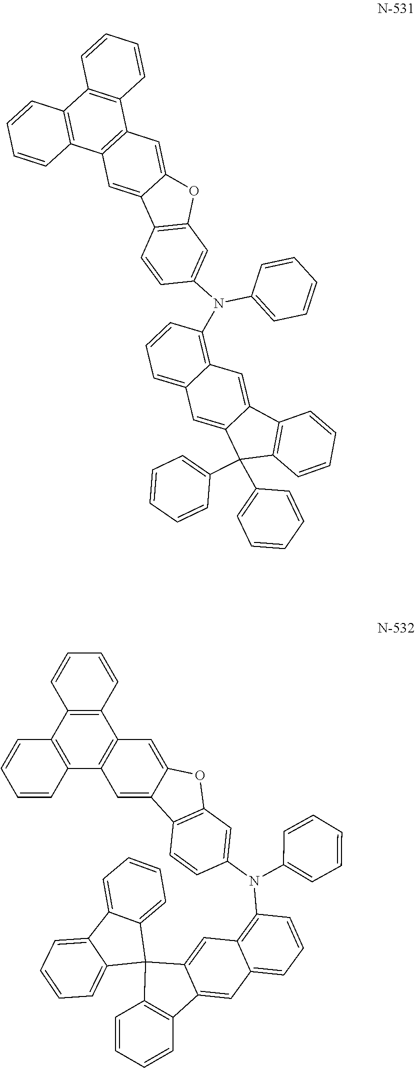 [SMILES: c1ccc(N(c2ccc3c(c2)oc2cc4c5ccccc5c5ccccc5c4cc23)c2cccc3cc4c(cc23)-c2ccccc2C4(c2ccccc2)c2ccccc2)cc1 ; c1ccc(N(c2ccc3c(c2)oc2cc4c5ccccc5c5ccccc5c4cc23)c2cccc3cc4c(cc23)C2(c3ccccc3-c3ccccc32)c2ccccc2-4)cc1]

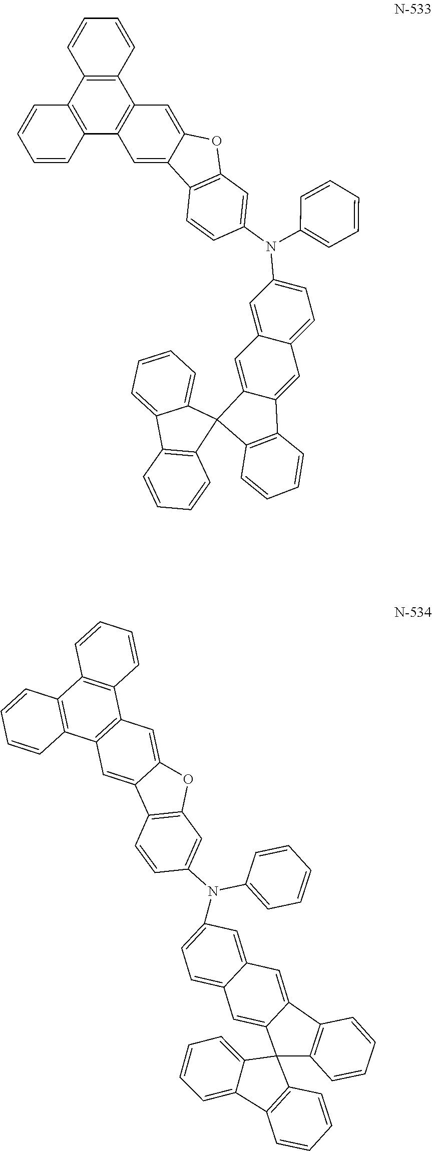 [SMILES: c1ccc(N(c2ccc3cc4c(cc3c2)C2(c3ccccc3-c3ccccc32)c2ccccc2-4)c2ccc3c(c2)oc2cc4c5ccccc5c5ccccc5c4cc23)cc1 ; c1ccc(N(c2ccc3cc4c(cc3c2)-c2ccccc2C42c3ccccc3-c3ccccc32)c2ccc3c(c2)oc2cc4c5ccccc5c5ccccc5c4cc23)cc1]

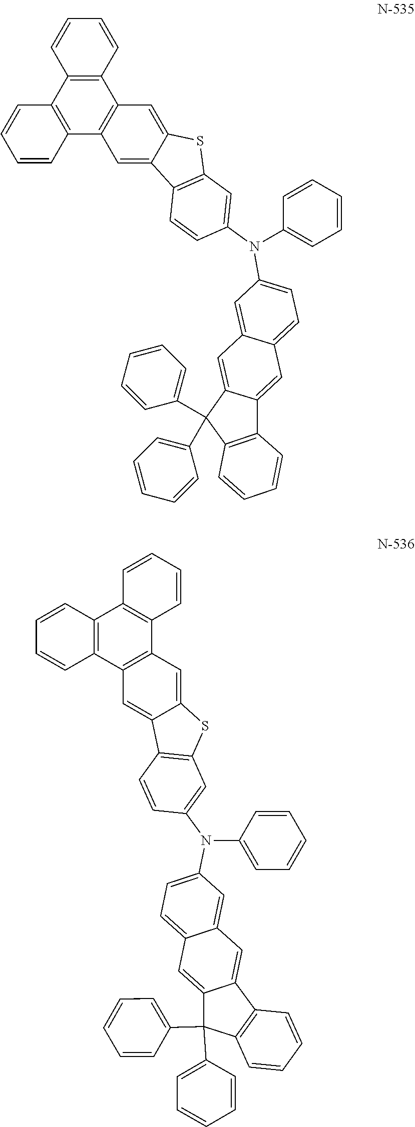 [SMILES: c1ccc(N(c2ccc3cc4c(cc3c2)C(c2ccccc2)(c2ccccc2)c2ccccc2-4)c2ccc3c(c2)sc2cc4c5ccccc5c5ccccc5c4cc23)cc1 ; c1ccc(N(c2ccc3cc4c(cc3c2)-c2ccccc2C4(c2ccccc2)c2ccccc2)c2ccc3c(c2)sc2cc4c5ccccc5c5ccccc5c4cc23)cc1]

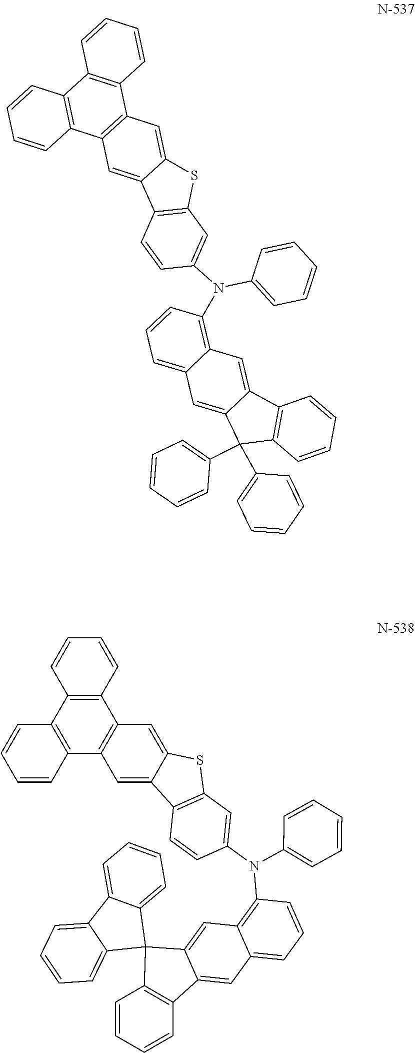 [SMILES: c1ccc(N(c2ccc3c(c2)sc2cc4c5ccccc5c5ccccc5c4cc23)c2cccc3cc4c(cc23)-c2ccccc2C4(c2ccccc2)c2ccccc2)cc1 ; c1ccc(N(c2ccc3c(c2)sc2cc4c5ccccc5c5ccccc5c4cc23)c2cccc3cc4c(cc23)C2(c3ccccc3-c3ccccc32)c2ccccc2-4)cc1]

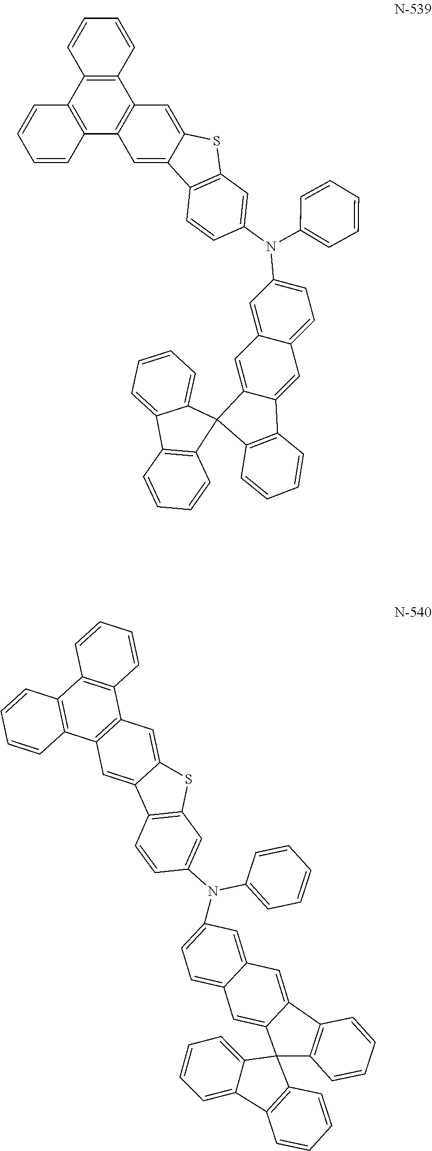 [SMILES: c1ccc(N(c2ccc3cc4c(cc3c2)C2(c3ccccc3-c3ccccc32)c2ccccc2-4)c2ccc3c(c2)sc2cc4c5ccccc5c5ccccc5c4cc23)cc1 ; c1ccc(N(c2ccc3cc4c(cc3c2)-c2ccccc2C42c3ccccc3-c3ccccc32)c2ccc3c(c2)sc2cc4c5ccccc5c5ccccc5c4cc23)cc1]

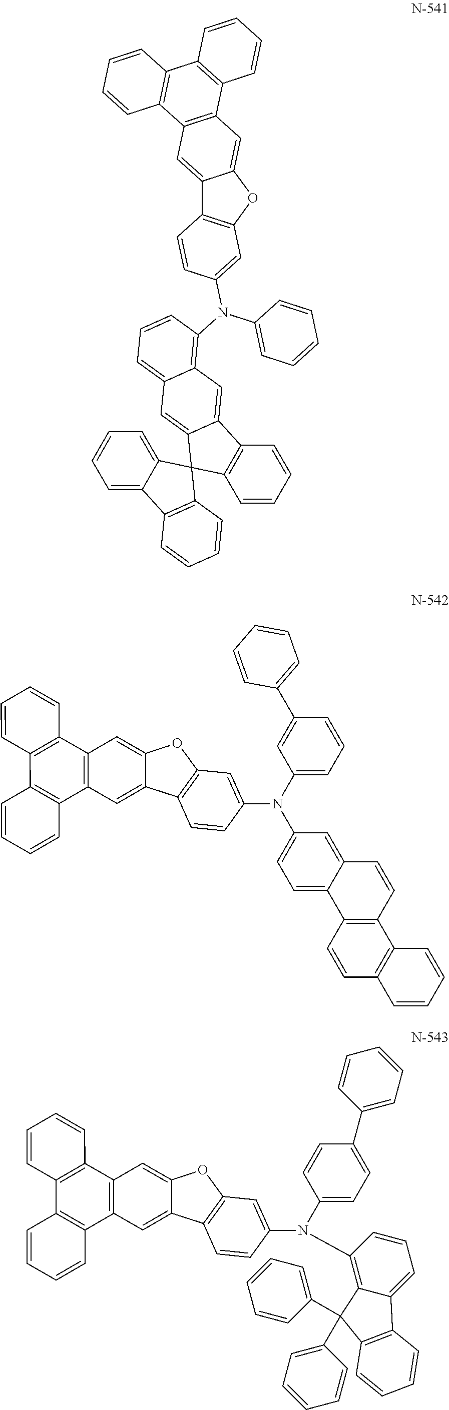

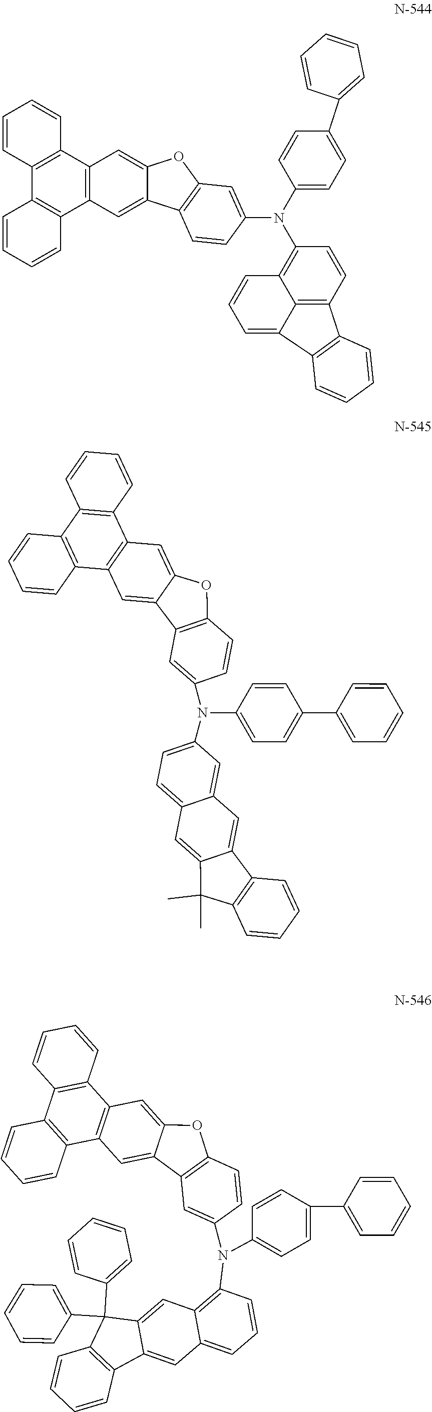 [SMILES: c1ccc(-c2ccc(N(c3ccc4c(c3)oc3cc5c6ccccc6c6ccccc6c5cc34)c3ccc4c5c(cccc35)-c3ccccc3-4)cc2)cc1 ; CC1(C)c2ccccc2-c2cc3cc(N(c4ccc(-c5ccccc5)cc4)c4ccc5oc6cc7c8ccccc8c8ccccc8c7cc6c5c4)ccc3cc21 ; c1ccc(-c2ccc(N(c3ccc4oc5cc6c7ccccc7c7ccccc7c6cc5c4c3)c3cccc4cc5c(cc34)C(c3ccccc3)(c3ccccc3)c3ccccc3-5)cc2)cc1]

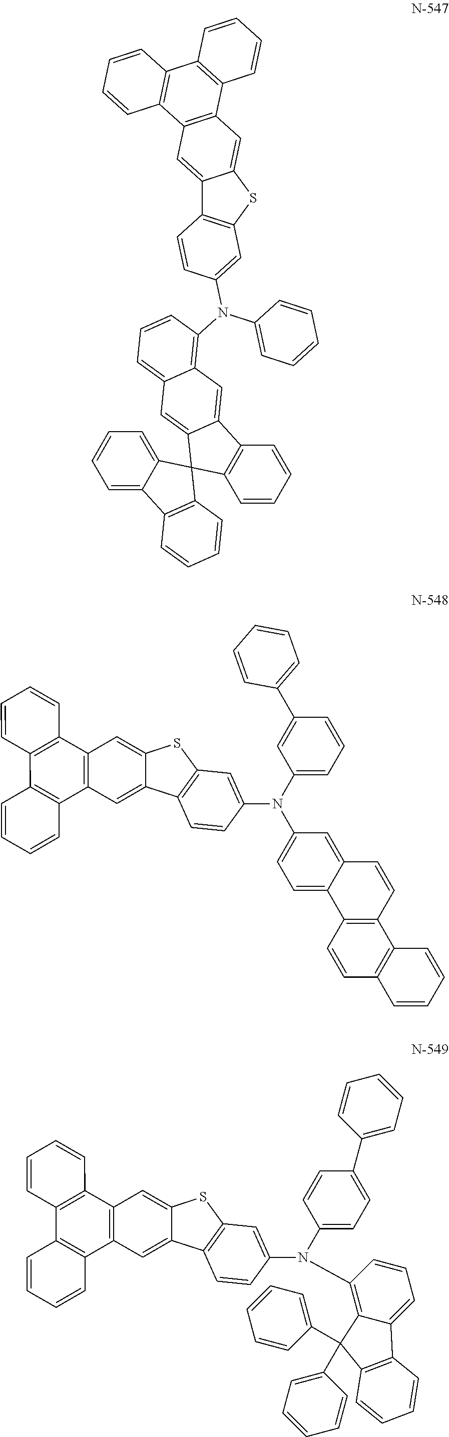 [SMILES: c1ccc(N(c2ccc3c(c2)sc2cc4c5ccccc5c5ccccc5c4cc23)c2cccc3cc4c(cc23)-c2ccccc2C42c3ccccc3-c3ccccc32)cc1 ; c1ccc(-c2cccc(N(c3ccc4c(ccc5c6ccccc6ccc45)c3)c3ccc4c(c3)sc3cc5c6ccccc6c6ccccc6c5cc34)c2)cc1 ; c1ccc(-c2ccc(N(c3ccc4c(c3)sc3cc5c6ccccc6c6ccccc6c5cc34)c3cccc4c3C(c3ccccc3)(c3ccccc3)c3ccccc3-4)cc2)cc1]

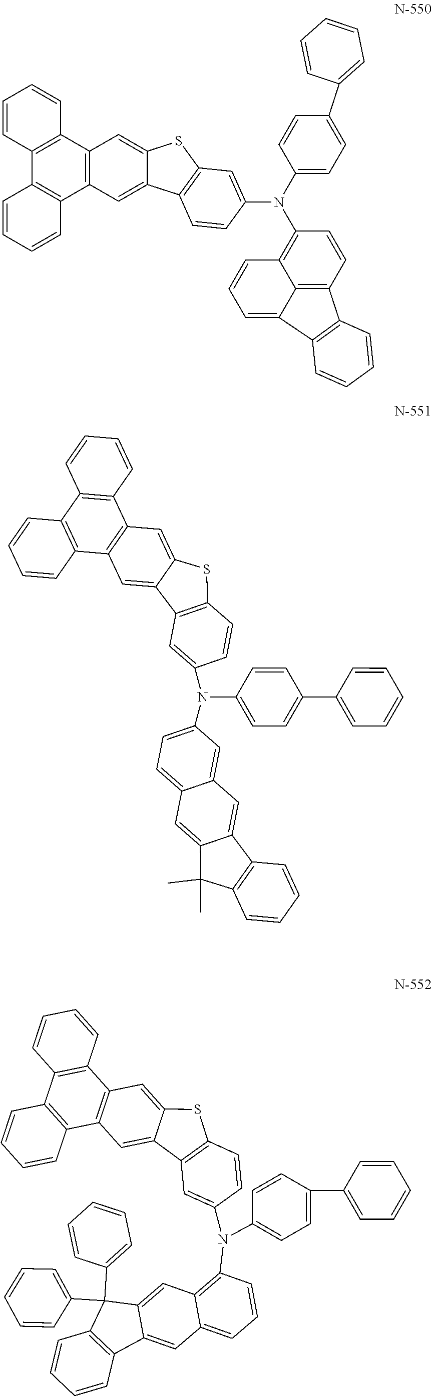 [SMILES: c1ccc(-c2ccc(N(c3ccc4c(c3)sc3cc5c6ccccc6c6ccccc6c5cc34)c3ccc4c5c(cccc35)-c3ccccc3-4)cc2)cc1 ; CC1(C)c2ccccc2-c2cc3cc(N(c4ccc(-c5ccccc5)cc4)c4ccc5sc6cc7c8ccccc8c8ccccc8c7cc6c5c4)ccc3cc21 ; c1ccc(-c2ccc(N(c3ccc4sc5cc6c7ccccc7c7ccccc7c6cc5c4c3)c3cccc4cc5c(cc34)C(c3ccccc3)(c3ccccc3)c3ccccc3-5)cc2)cc1]

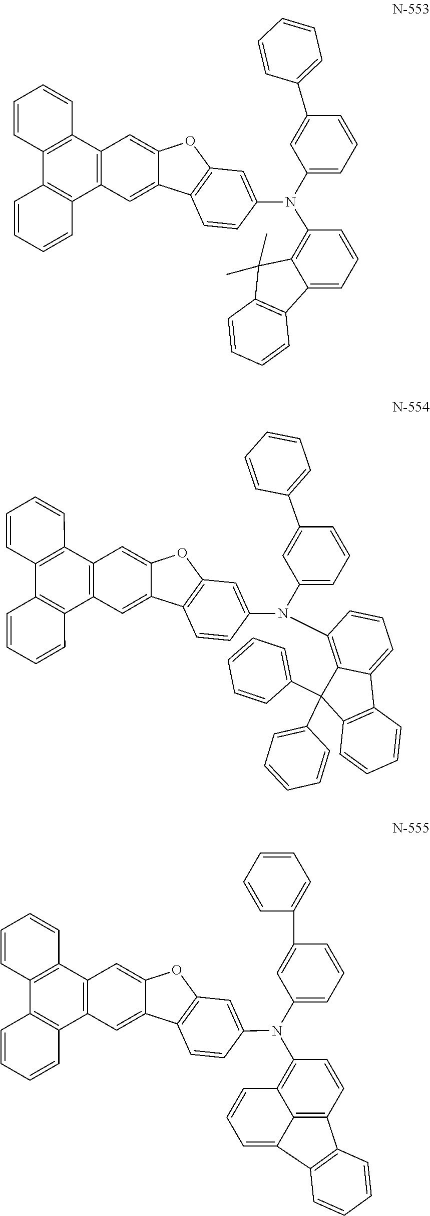 [SMILES: CC1(C)c2ccccc2-c2cccc(N(c3cccc(-c4ccccc4)c3)c3ccc4c(c3)oc3cc5c6ccccc6c6ccccc6c5cc34)c21 ; c1ccc(-c2cccc(N(c3ccc4c(c3)oc3cc5c6ccccc6c6ccccc6c5cc34)c3cccc4c3C(c3ccccc3)(c3ccccc3)c3ccccc3-4)c2)cc1 ; c1ccc(-c2cccc(N(c3ccc4c(c3)oc3cc5c6ccccc6c6ccccc6c5cc34)c3ccc4c5c(cccc35)-c3ccccc3-4)c2)cc1]

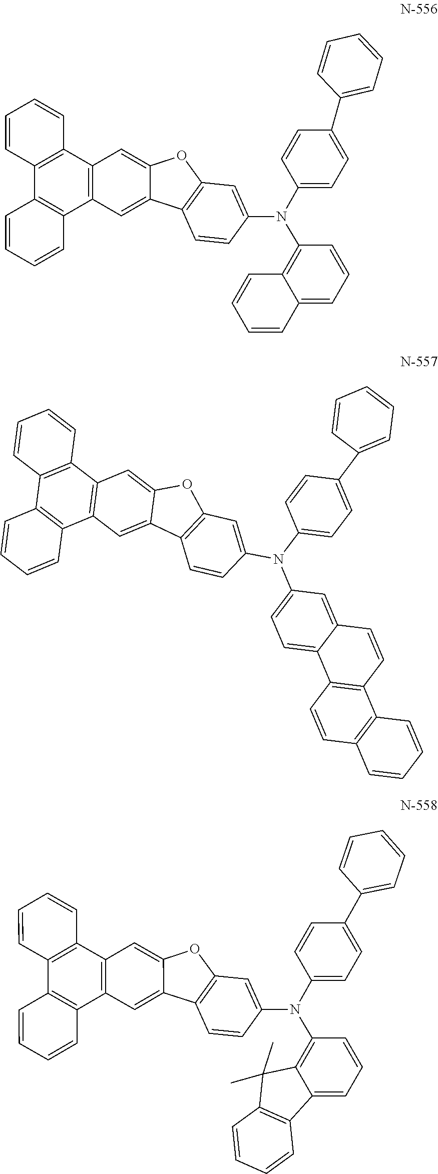 [SMILES: c1ccc(-c2ccc(N(c3ccc4c(c3)oc3cc5c6ccccc6c6ccccc6c5cc34)c3cccc4ccccc34)cc2)cc1 ; c1ccc(-c2ccc(N(c3ccc4c(ccc5c6ccccc6ccc45)c3)c3ccc4c(c3)oc3cc5c6ccccc6c6ccccc6c5cc34)cc2)cc1 ; CC1(C)c2ccccc2-c2cccc(N(c3ccc(-c4ccccc4)cc3)c3ccc4c(c3)oc3cc5c6ccccc6c6ccccc6c5cc34)c21]

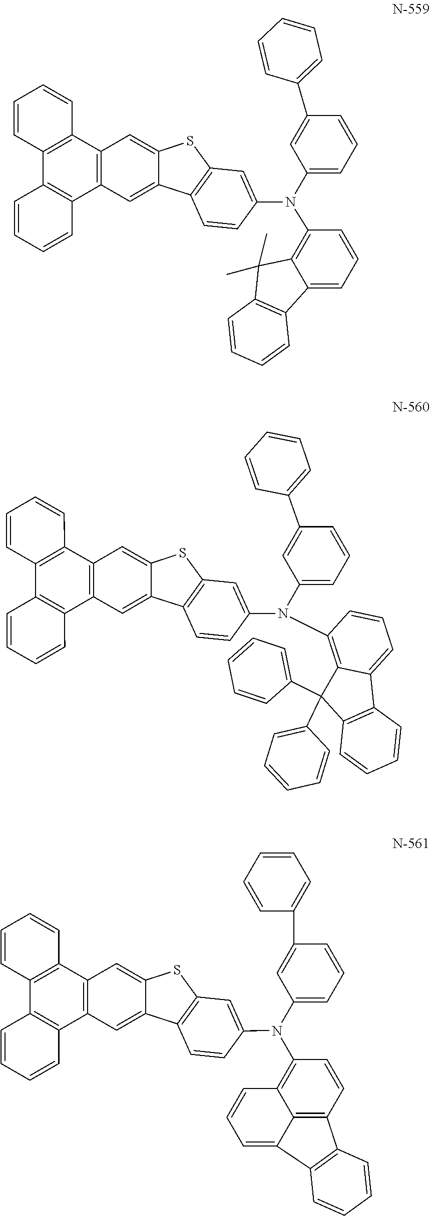 [SMILES: CC1(C)c2ccccc2-c2cccc(N(c3cccc(-c4ccccc4)c3)c3ccc4c(c3)sc3cc5c6ccccc6c6ccccc6c5cc34)c21 ; c1ccc(-c2cccc(N(c3ccc4c(c3)sc3cc5c6ccccc6c6ccccc6c5cc34)c3cccc4c3C(c3ccccc3)(c3ccccc3)c3ccccc3-4)c2)cc1 ; c1ccc(-c2cccc(N(c3ccc4c(c3)sc3cc5c6ccccc6c6ccccc6c5cc34)c3ccc4c5c(cccc35)-c3ccccc3-4)c2)cc1]

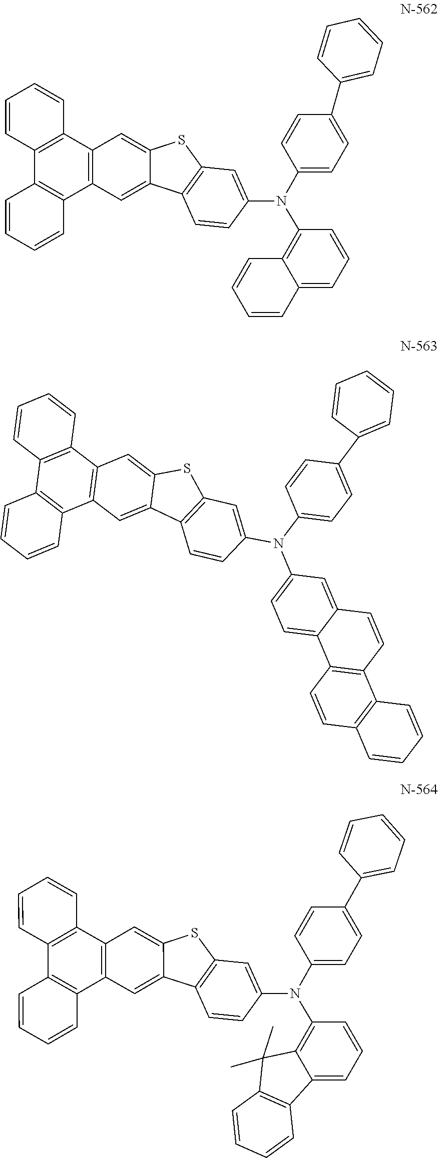 [SMILES: c1ccc(-c2ccc(N(c3ccc4c(c3)sc3cc5c6ccccc6c6ccccc6c5cc34)c3cccc4ccccc34)cc2)cc1 ; c1ccc(-c2ccc(N(c3ccc4c(ccc5c6ccccc6ccc45)c3)c3ccc4c(c3)sc3cc5c6ccccc6c6ccccc6c5cc34)cc2)cc1 ; CC1(C)c2ccccc2-c2cccc(N(c3ccc(-c4ccccc4)cc3)c3ccc4c(c3)sc3cc5c6ccccc6c6ccccc6c5cc34)c21]

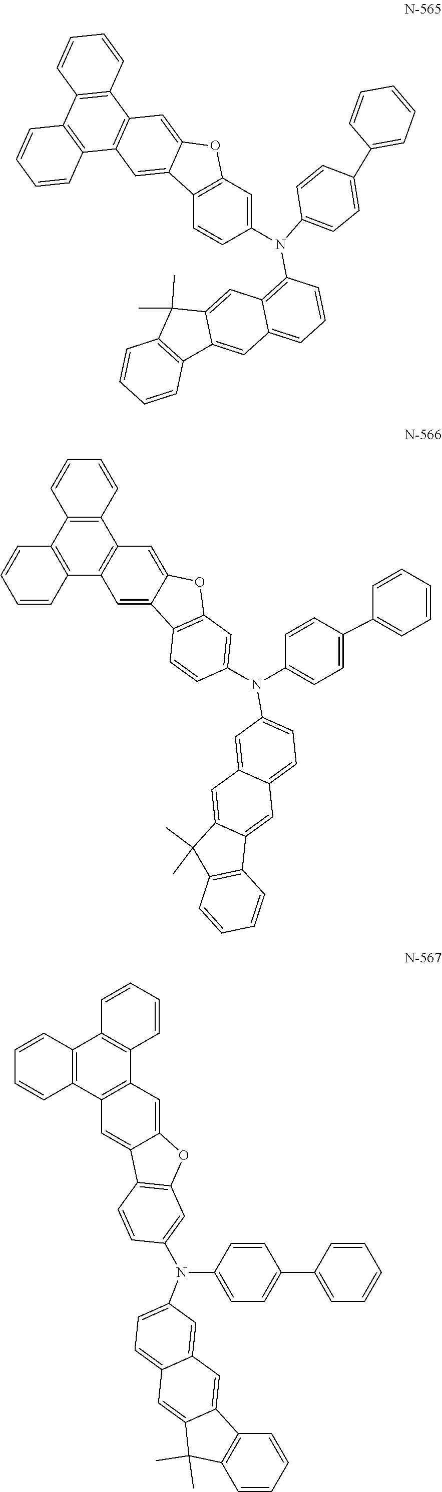 [SMILES: CC1(C)c2ccccc2-c2cc3cccc(N(c4ccc(-c5ccccc5)cc4)c4ccc5c(c4)oc4cc6c7ccccc7c7ccccc7c6cc45)c3cc21 ; CC1(C)c2ccccc2-c2cc3ccc(N(c4ccc(-c5ccccc5)cc4)c4ccc5c(c4)oc4cc6c7ccccc7c7ccccc7c6cc45)cc3cc21 ; CC1(C)c2ccccc2-c2cc3cc(N(c4ccc(-c5ccccc5)cc4)c4ccc5c(c4)oc4cc6c7ccccc7c7ccccc7c6cc45)ccc3cc21]

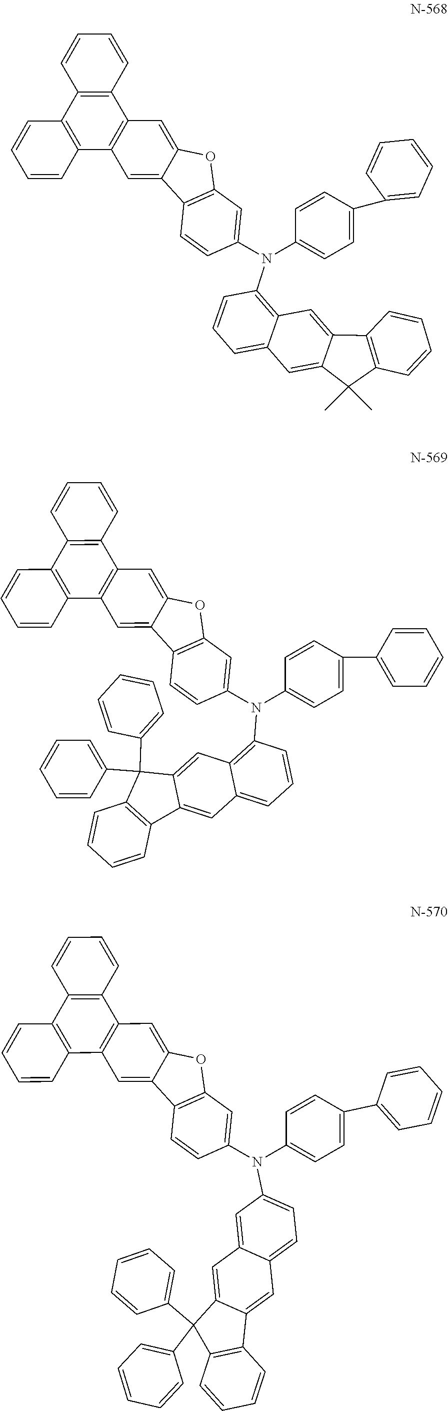 [SMILES: CC1(C)c2ccccc2-c2cc3c(N(c4ccc(-c5ccccc5)cc4)c4ccc5c(c4)oc4cc6c7ccccc7c7ccccc7c6cc45)cccc3cc21 ; c1ccc(-c2ccc(N(c3ccc4c(c3)oc3cc5c6ccccc6c6ccccc6c5cc34)c3cccc4cc5c(cc34)C(c3ccccc3)(c3ccccc3)c3ccccc3-5)cc2)cc1 ; c1ccc(-c2ccc(N(c3ccc4cc5c(cc4c3)C(c3ccccc3)(c3ccccc3)c3ccccc3-5)c3ccc4c(c3)oc3cc5c6ccccc6c6ccccc6c5cc34)cc2)cc1]

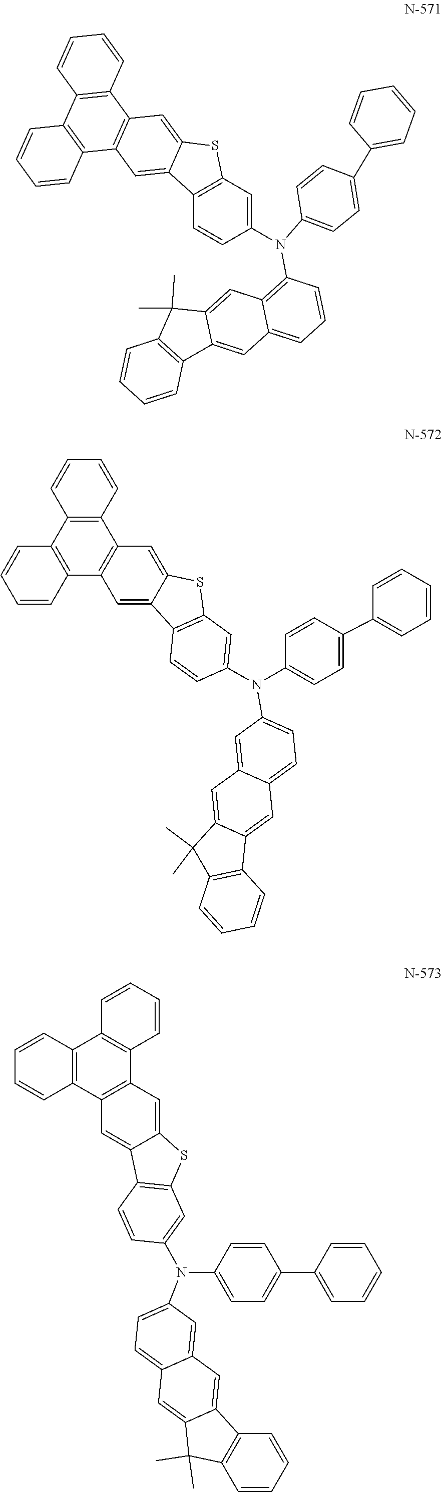 [SMILES: CC1(C)c2ccccc2-c2cc3cccc(N(c4ccc(-c5ccccc5)cc4)c4ccc5c(c4)sc4cc6c7ccccc7c7ccccc7c6cc45)c3cc21 ; CC1(C)c2ccccc2-c2cc3ccc(N(c4ccc(-c5ccccc5)cc4)c4ccc5c(c4)sc4cc6c7ccccc7c7ccccc7c6cc45)cc3cc21 ; CC1(C)c2ccccc2-c2cc3cc(N(c4ccc(-c5ccccc5)cc4)c4ccc5c(c4)sc4cc6c7ccccc7c7ccccc7c6cc45)ccc3cc21]

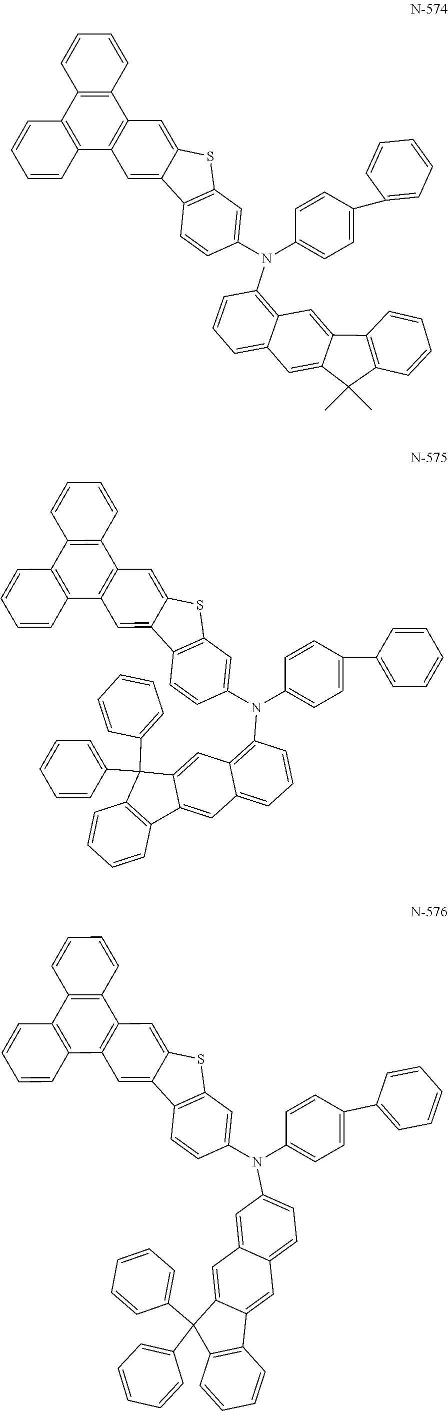 [SMILES: CC1(C)c2ccccc2-c2cc3c(N(c4ccc(-c5ccccc5)cc4)c4ccc5c(c4)sc4cc6c7ccccc7c7ccccc7c6cc45)cccc3cc21 ; c1ccc(-c2ccc(N(c3ccc4c(c3)sc3cc5c6ccccc6c6ccccc6c5cc34)c3cccc4cc5c(cc34)C(c3ccccc3)(c3ccccc3)c3ccccc3-5)cc2)cc1 ; c1ccc(-c2ccc(N(c3ccc4cc5c(cc4c3)C(c3ccccc3)(c3ccccc3)c3ccccc3-5)c3ccc4c(c3)sc3cc5c6ccccc6c6ccccc6c5cc34)cc2)cc1]

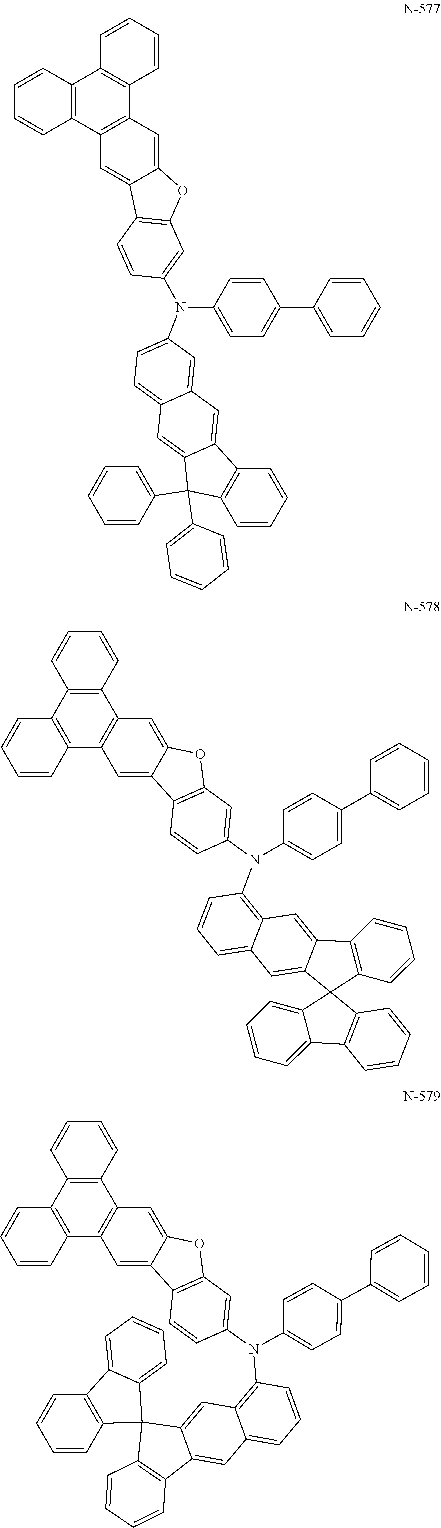 [SMILES: c1ccc(-c2ccc(N(c3ccc4cc5c(cc4c3)-c3ccccc3C5(c3ccccc3)c3ccccc3)c3ccc4c(c3)oc3cc5c6ccccc6c6ccccc6c5cc34)cc2)cc1 ; c1ccc(-c2ccc(N(c3ccc4c(c3)oc3cc5c6ccccc6c6ccccc6c5cc34)c3cccc4cc5c(cc34)-c3ccccc3C53c4ccccc4-c4ccccc43)cc2)cc1 ; c1ccc(-c2ccc(N(c3ccc4c(c3)oc3cc5c6ccccc6c6ccccc6c5cc34)c3cccc4cc5c(cc34)C3(c4ccccc4-c4ccccc43)c3ccccc3-5)cc2)cc1]

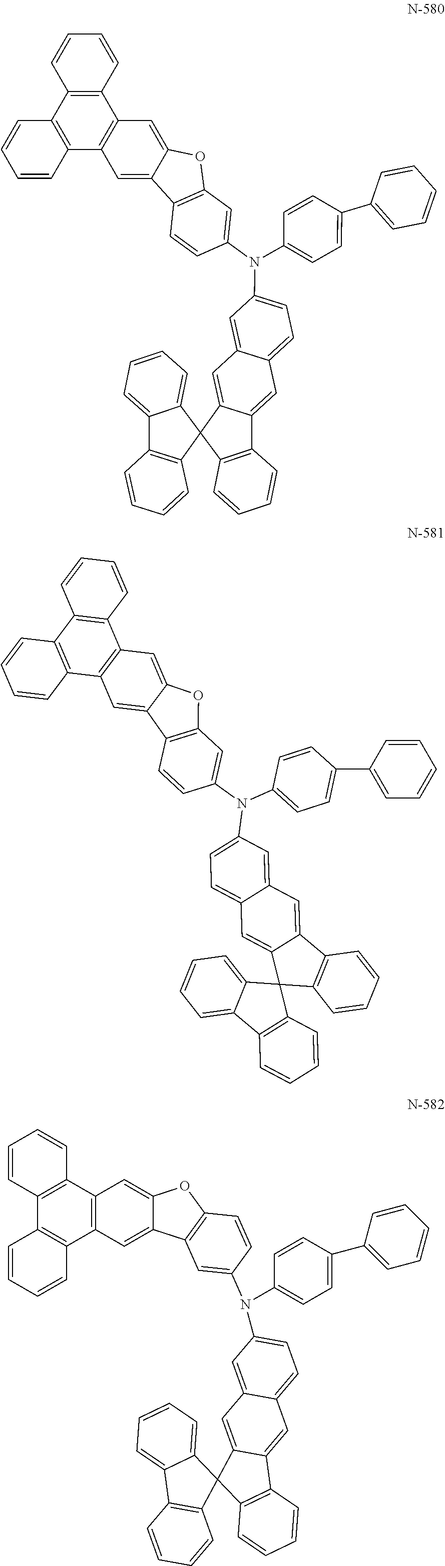 [SMILES: c1ccc(-c2ccc(N(c3ccc4cc5c(cc4c3)C3(c4ccccc4-c4ccccc43)c3ccccc3-5)c3ccc4c(c3)oc3cc5c6ccccc6c6ccccc6c5cc34)cc2)cc1 ; c1ccc(-c2ccc(N(c3ccc4cc5c(cc4c3)-c3ccccc3C53c4ccccc4-c4ccccc43)c3ccc4c(c3)oc3cc5c6ccccc6c6ccccc6c5cc34)cc2)cc1 ; c1ccc(-c2ccc(N(c3ccc4cc5c(cc4c3)C3(c4ccccc4-c4ccccc43)c3ccccc3-5)c3ccc4oc5cc6c7ccccc7c7ccccc7c6cc5c4c3)cc2)cc1]

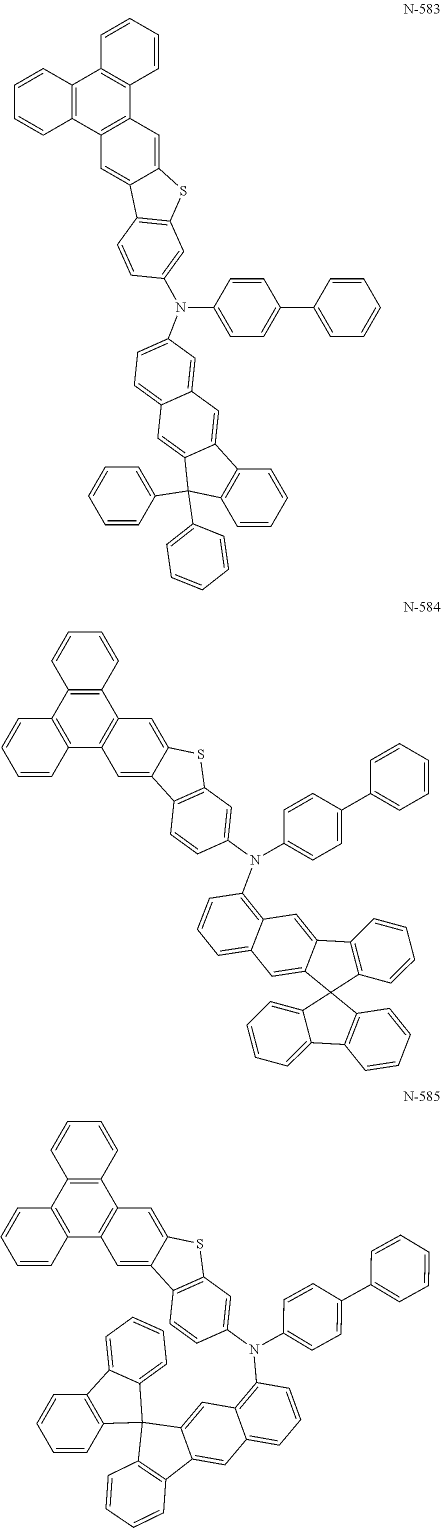 [SMILES: c1ccc(-c2ccc(N(c3ccc4cc5c(cc4c3)-c3ccccc3C5(c3ccccc3)c3ccccc3)c3ccc4c(c3)sc3cc5c6ccccc6c6ccccc6c5cc34)cc2)cc1 ; c1ccc(-c2ccc(N(c3ccc4c(c3)sc3cc5c6ccccc6c6ccccc6c5cc34)c3cccc4cc5c(cc34)-c3ccccc3C53c4ccccc4-c4ccccc43)cc2)cc1 ; c1ccc(-c2ccc(N(c3ccc4c(c3)sc3cc5c6ccccc6c6ccccc6c5cc34)c3cccc4cc5c(cc34)C3(c4ccccc4-c4ccccc43)c3ccccc3-5)cc2)cc1]

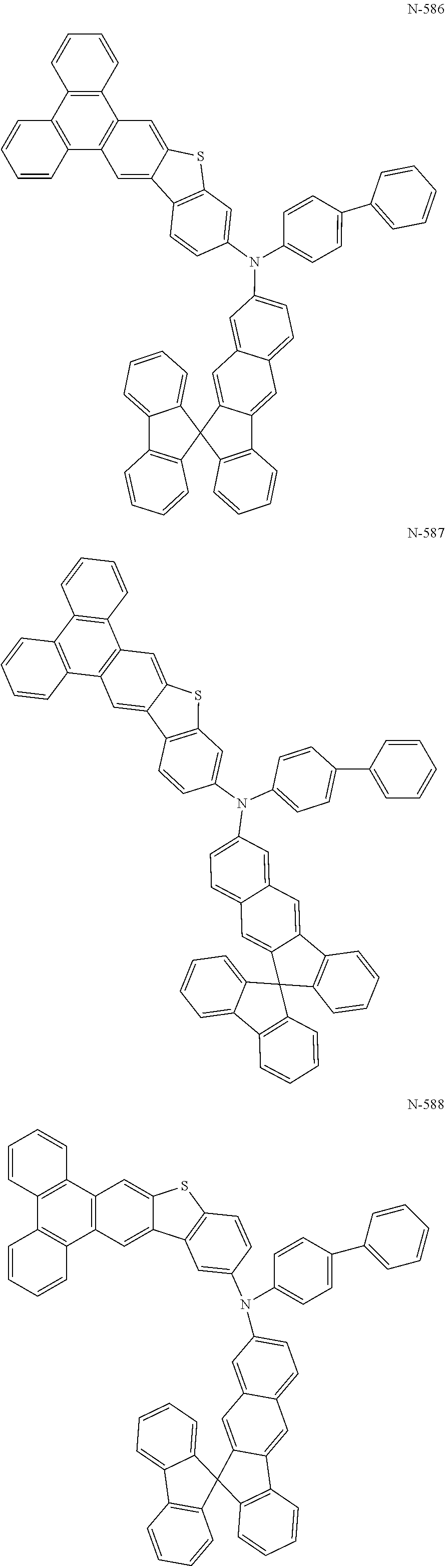 [SMILES: c1ccc(-c2ccc(N(c3ccc4cc5c(cc4c3)C3(c4ccccc4-c4ccccc43)c3ccccc3-5)c3ccc4c(c3)sc3cc5c6ccccc6c6ccccc6c5cc34)cc2)cc1 ; c1ccc(-c2ccc(N(c3ccc4cc5c(cc4c3)-c3ccccc3C53c4ccccc4-c4ccccc43)c3ccc4c(c3)sc3cc5c6ccccc6c6ccccc6c5cc34)cc2)cc1 ; c1ccc(-c2ccc(N(c3ccc4cc5c(cc4c3)C3(c4ccccc4-c4ccccc43)c3ccccc3-5)c3ccc4sc5cc6c7ccccc7c7ccccc7c6cc5c4c3)cc2)cc1]

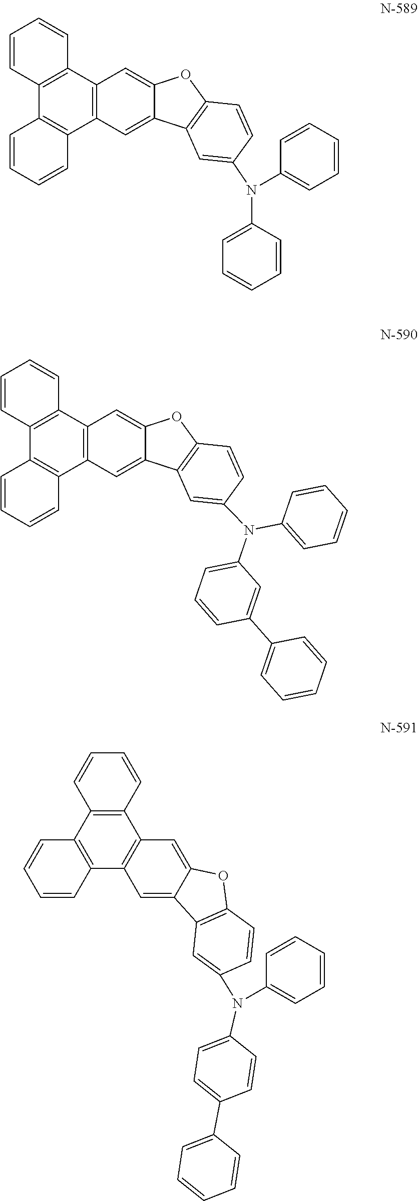 [SMILES: c1ccc(N(c2ccccc2)c2ccc3oc4cc5c6ccccc6c6ccccc6c5cc4c3c2)cc1 ; c1ccc(-c2cccc(N(c3ccccc3)c3ccc4oc5cc6c7ccccc7c7ccccc7c6cc5c4c3)c2)cc1 ; c1ccc(-c2ccc(N(c3ccccc3)c3ccc4oc5cc6c7ccccc7c7ccccc7c6cc5c4c3)cc2)cc1]

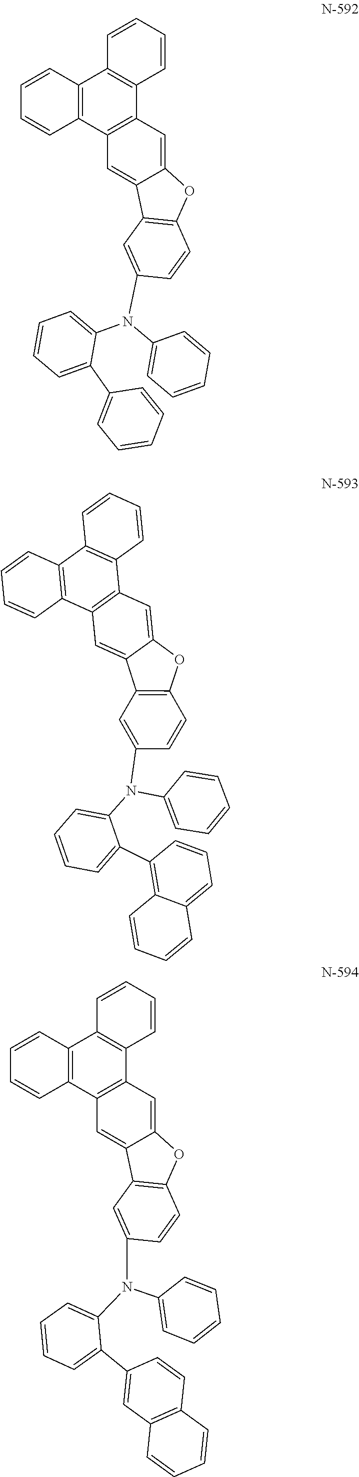

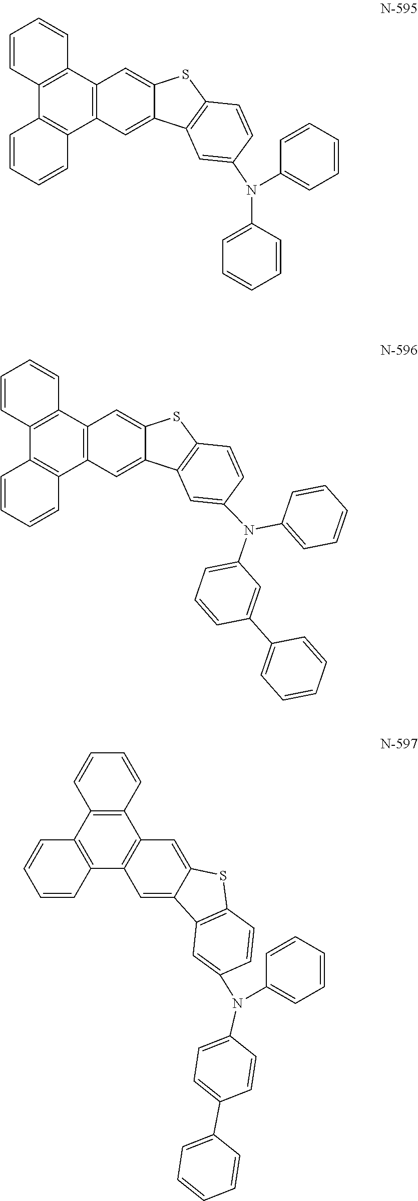 [SMILES: c1ccc(N(c2ccccc2)c2ccc3sc4cc5c6ccccc6c6ccccc6c5cc4c3c2)cc1 ; c1ccc(-c2cccc(N(c3ccccc3)c3ccc4sc5cc6c7ccccc7c7ccccc7c6cc5c4c3)c2)cc1 ; c1ccc(-c2ccc(N(c3ccccc3)c3ccc4sc5cc6c7ccccc7c7ccccc7c6cc5c4c3)cc2)cc1]

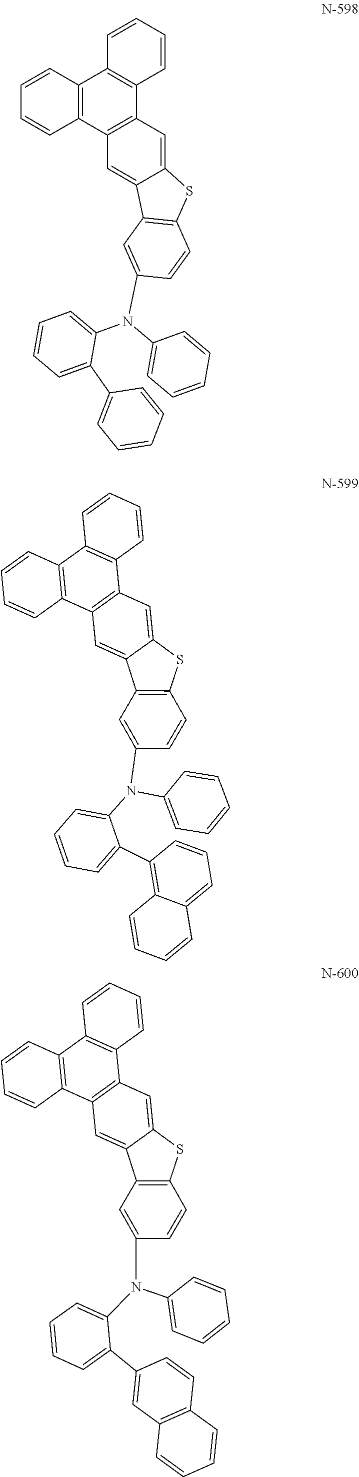

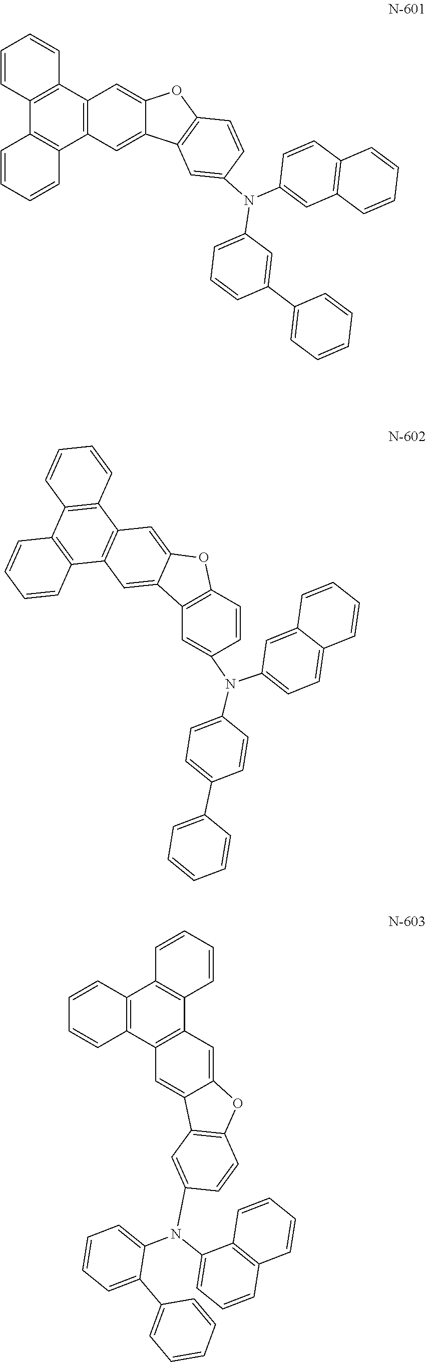 [SMILES: c1ccc(-c2cccc(N(c3ccc4ccccc4c3)c3ccc4oc5cc6c7ccccc7c7ccccc7c6cc5c4c3)c2)cc1 ; c1ccc(-c2ccc(N(c3ccc4ccccc4c3)c3ccc4oc5cc6c7ccccc7c7ccccc7c6cc5c4c3)cc2)cc1 ; c1ccc(-c2ccccc2N(c2ccc3oc4cc5c6ccccc6c6ccccc6c5cc4c3c2)c2cccc3ccccc23)cc1]

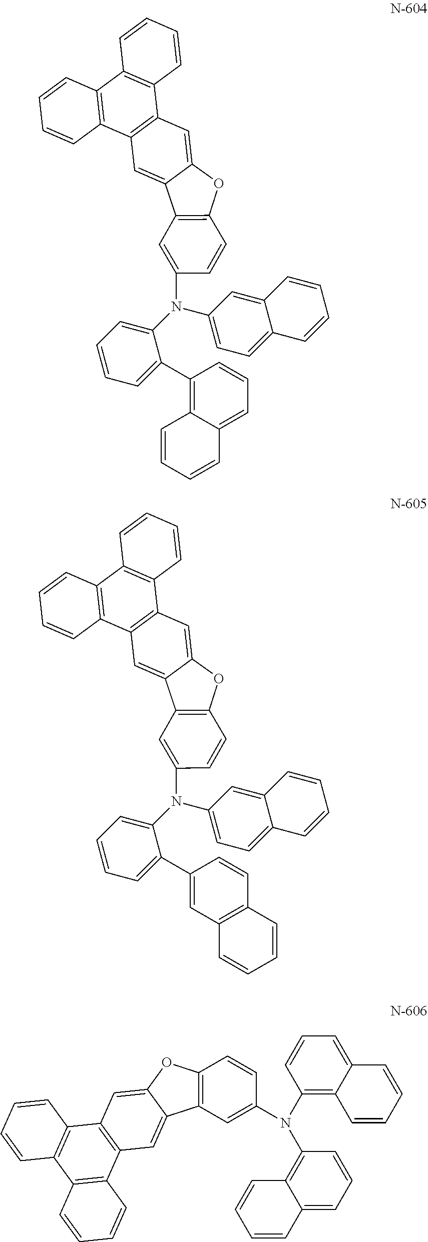 [SMILES: c1ccc(N(c2ccc3ccccc3c2)c2ccc3oc4cc5c6ccccc6c6ccccc6c5cc4c3c2)c(-c2cccc3ccccc23)c1 ; c1ccc(N(c2ccc3ccccc3c2)c2ccc3oc4cc5c6ccccc6c6ccccc6c5cc4c3c2)c(-c2ccc3ccccc3c2)c1 ; c1ccc2c(N(c3ccc4oc5cc6c7ccccc7c7ccccc7c6cc5c4c3)c3cccc4ccccc34)cccc2c1]

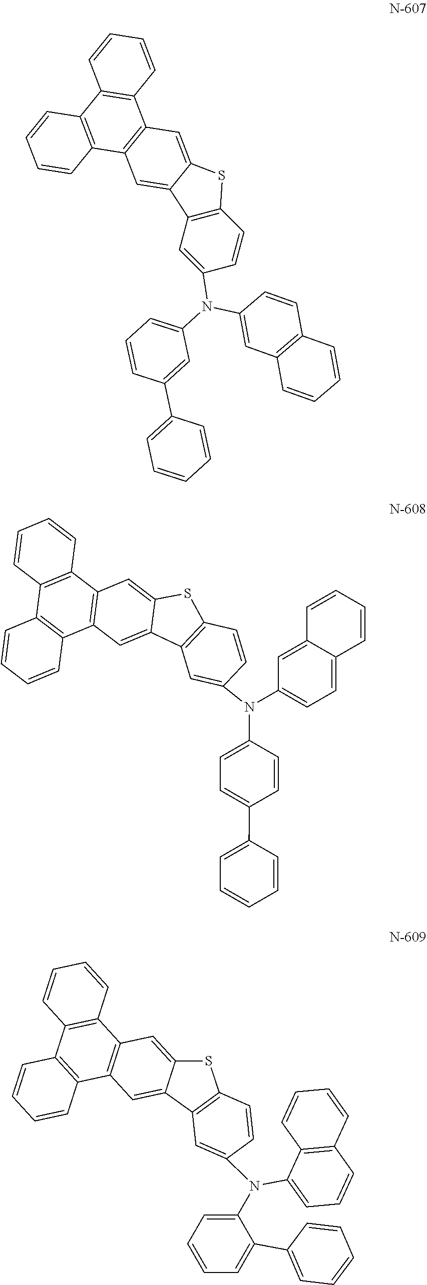 [SMILES: c1ccc(-c2cccc(N(c3ccc4ccccc4c3)c3ccc4sc5cc6c7ccccc7c7ccccc7c6cc5c4c3)c2)cc1 ; c1ccc(-c2ccc(N(c3ccc4ccccc4c3)c3ccc4sc5cc6c7ccccc7c7ccccc7c6cc5c4c3)cc2)cc1 ; c1ccc(-c2ccccc2N(c2ccc3sc4cc5c6ccccc6c6ccccc6c5cc4c3c2)c2cccc3ccccc23)cc1]

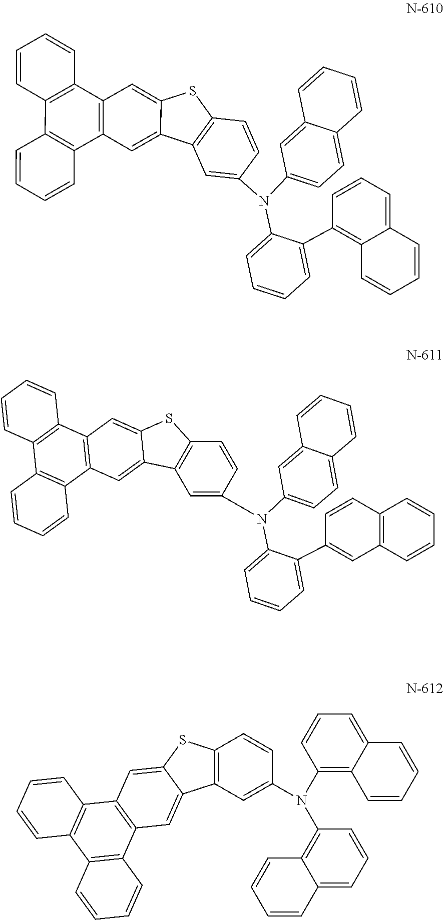 [SMILES: c1ccc(N(c2ccc3ccccc3c2)c2ccc3sc4cc5c6ccccc6c6ccccc6c5cc4c3c2)c(-c2cccc3ccccc23)c1 ; c1ccc(N(c2ccc3ccccc3c2)c2ccc3sc4cc5c6ccccc6c6ccccc6c5cc4c3c2)c(-c2ccc3ccccc3c2)c1 ; c1ccc2c(N(c3ccc4sc5cc6c7ccccc7c7ccccc7c6cc5c4c3)c3cccc4ccccc34)cccc2c1]

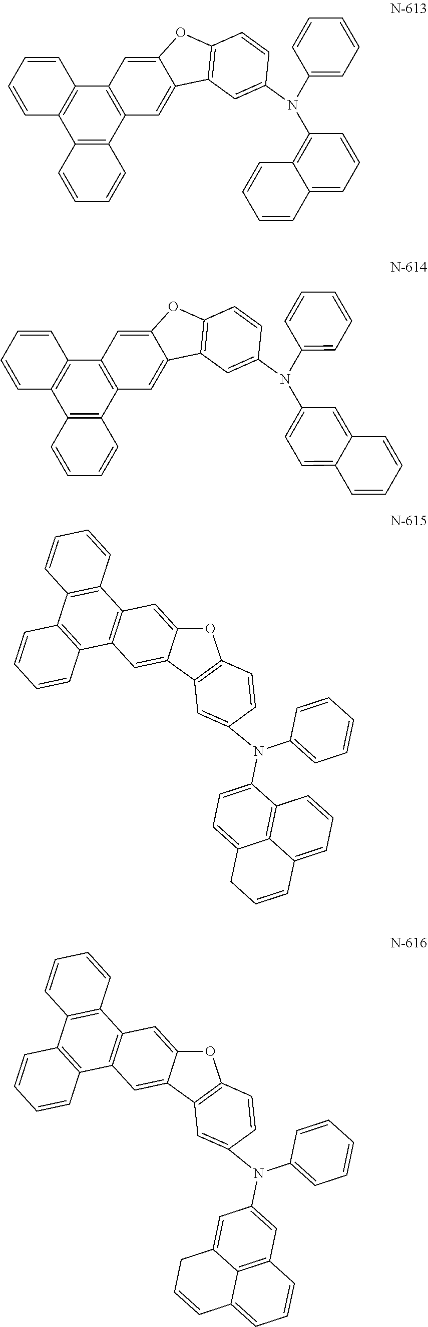 [SMILES: c1ccc(N(c2ccc3oc4cc5c6ccccc6c6ccccc6c5cc4c3c2)c2cccc3ccccc23)cc1 ; c1ccc(N(c2ccc3ccccc3c2)c2ccc3oc4cc5c6ccccc6c6ccccc6c5cc4c3c2)cc1 ; C1=Cc2cccc3c(N(c4ccccc4)c4ccc5oc6cc7c8ccccc8c8ccccc8c7cc6c5c4)ccc(c23)C1 ; C1=Cc2cccc3cc(N(c4ccccc4)c4ccc5oc6cc7c8ccccc8c8ccccc8c7cc6c5c4)cc(c23)C1]

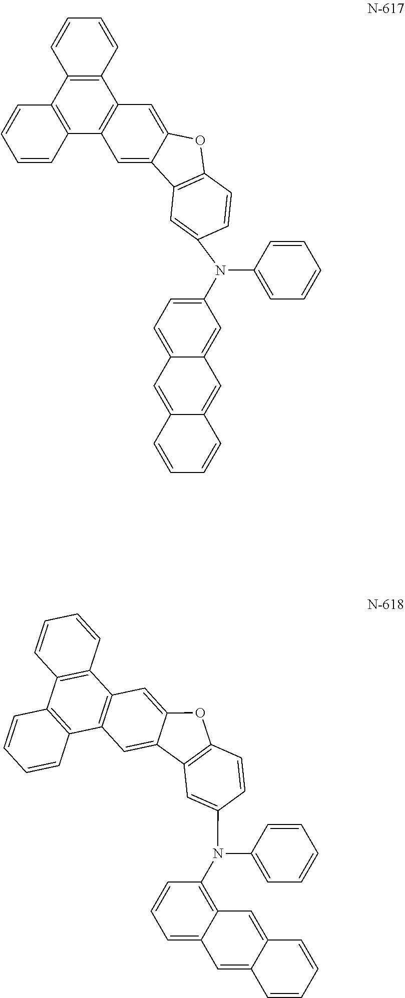 [SMILES: c1ccc(N(c2ccc3cc4ccccc4cc3c2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2cccc3cc4ccccc4cc23)cc1]

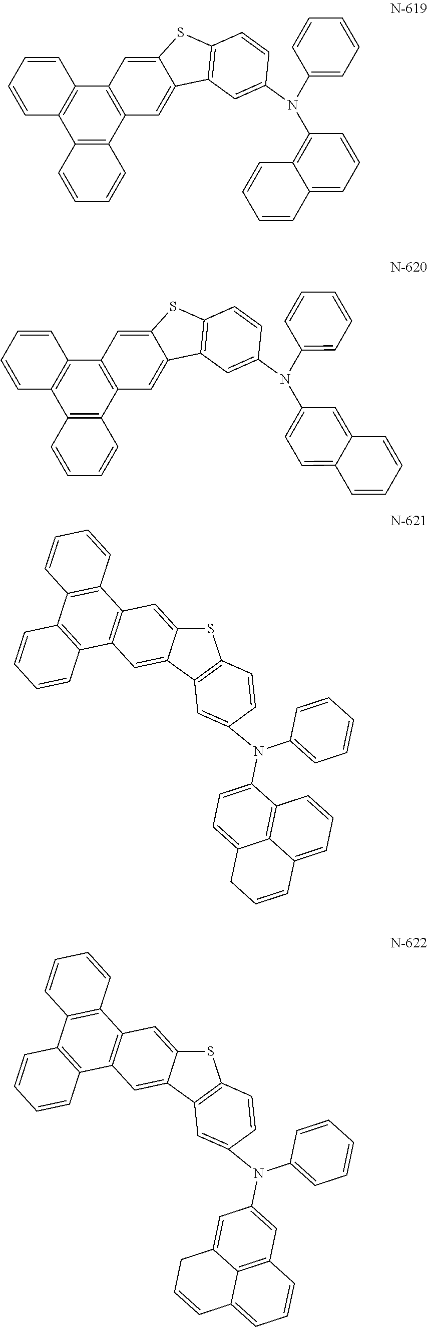 [SMILES: c1ccc(N(c2ccc3sc4cc5c6ccccc6c6ccccc6c5cc4c3c2)c2cccc3ccccc23)cc1 ; c1ccc(N(c2ccc3ccccc3c2)c2ccc3sc4cc5c6ccccc6c6ccccc6c5cc4c3c2)cc1 ; C1=Cc2cccc3c(N(c4ccccc4)c4ccc5sc6cc7c8ccccc8c8ccccc8c7cc6c5c4)ccc(c23)C1 ; C1=Cc2cccc3cc(N(c4ccccc4)c4ccc5sc6cc7c8ccccc8c8ccccc8c7cc6c5c4)cc(c23)C1]

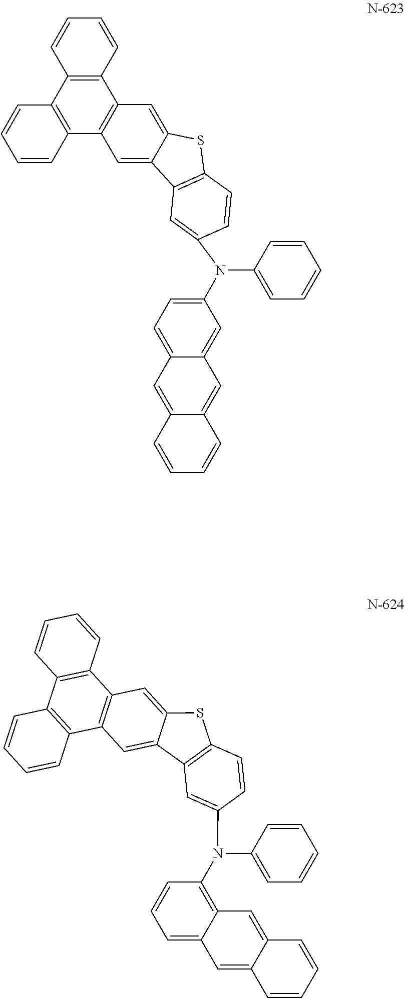 [SMILES: c1ccc(N(c2ccc3cc4ccccc4cc3c2)c2ccc3sc4cc5c6ccccc6c6ccccc6c5cc4c3c2)cc1 ; c1ccc(N(c2ccc3sc4cc5c6ccccc6c6ccccc6c5cc4c3c2)c2cccc3cc4ccccc4cc23)cc1]

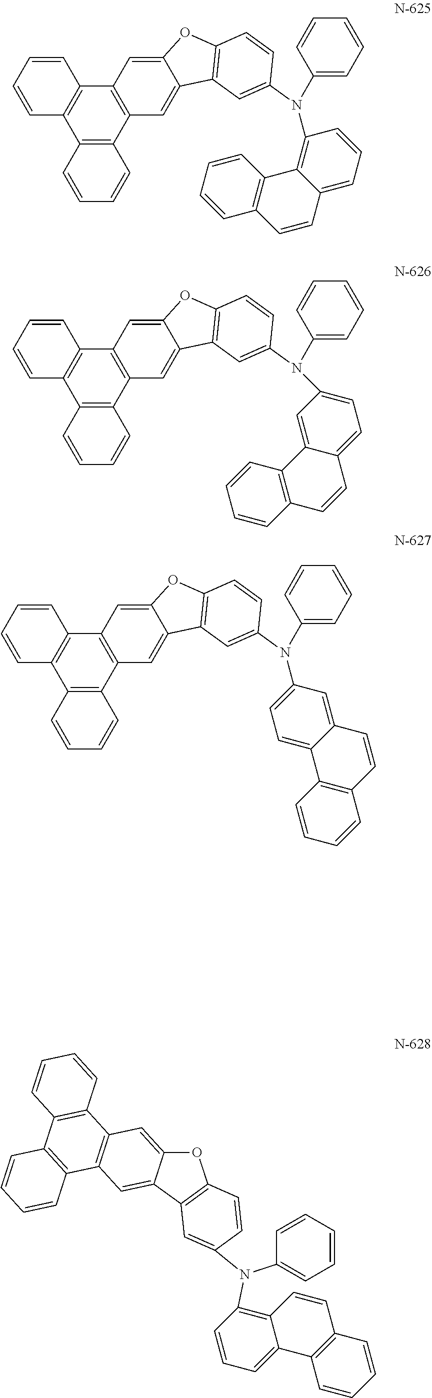 [SMILES: c1ccc(N(c2ccc3oc4cc5c6ccccc6c6ccccc6c5cc4c3c2)c2cccc3ccc4ccccc4c23)cc1 ; c1ccc(N(c2ccc3ccc4ccccc4c3c2)c2ccc3oc4cc5c6ccccc6c6ccccc6c5cc4c3c2)cc1 ; c1ccc(N(c2ccc3c(ccc4ccccc43)c2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2cccc3c2ccc2ccccc23)cc1]

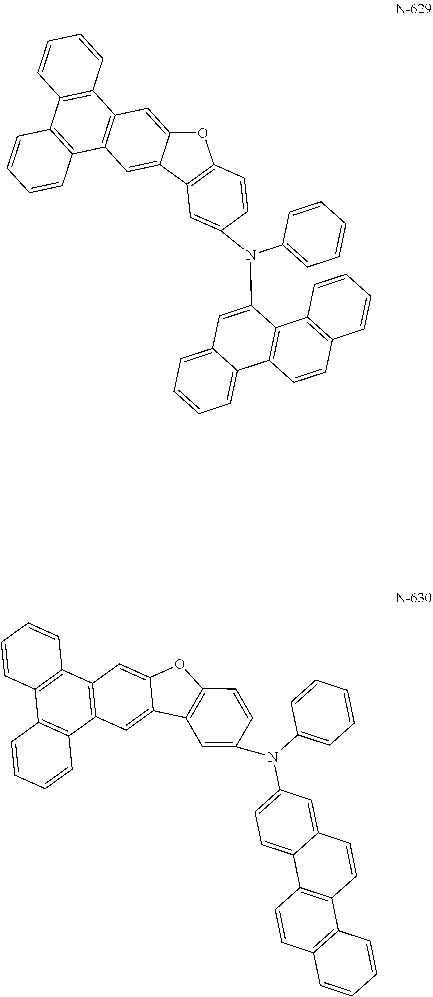 [SMILES: c1ccc(N(c2ccc3oc4cc5c6ccccc6c6ccccc6c5cc4c3c2)c2cc3ccccc3c3ccc4ccccc4c23)cc1 ; c1ccc(N(c2ccc3c(ccc4c5ccccc5ccc34)c2)c2ccc3oc4cc5c6ccccc6c6ccccc6c5cc4c3c2)cc1]

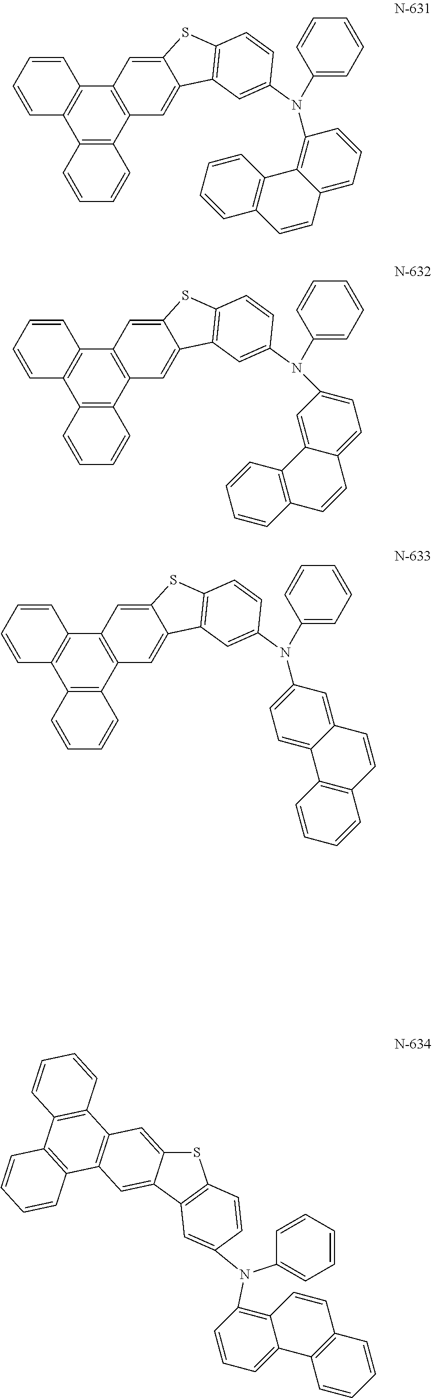 [SMILES: c1ccc(N(c2ccc3sc4cc5c6ccccc6c6ccccc6c5cc4c3c2)c2cccc3ccc4ccccc4c23)cc1 ; c1ccc(N(c2ccc3ccc4ccccc4c3c2)c2ccc3sc4cc5c6ccccc6c6ccccc6c5cc4c3c2)cc1 ; c1ccc(N(c2ccc3c(ccc4ccccc43)c2)c2ccc3sc4cc5c6ccccc6c6ccccc6c5cc4c3c2)cc1 ; c1ccc(N(c2ccc3sc4cc5c6ccccc6c6ccccc6c5cc4c3c2)c2cccc3c2ccc2ccccc23)cc1]

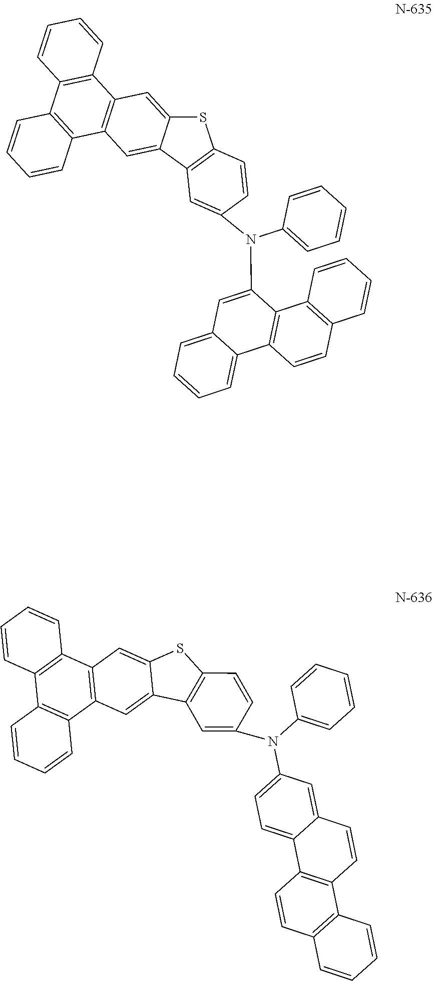 [SMILES: c1ccc(N(c2ccc3sc4cc5c6ccccc6c6ccccc6c5cc4c3c2)c2cc3ccccc3c3ccc4ccccc4c23)cc1 ; c1ccc(N(c2ccc3c(ccc4c5ccccc5ccc34)c2)c2ccc3sc4cc5c6ccccc6c6ccccc6c5cc4c3c2)cc1]

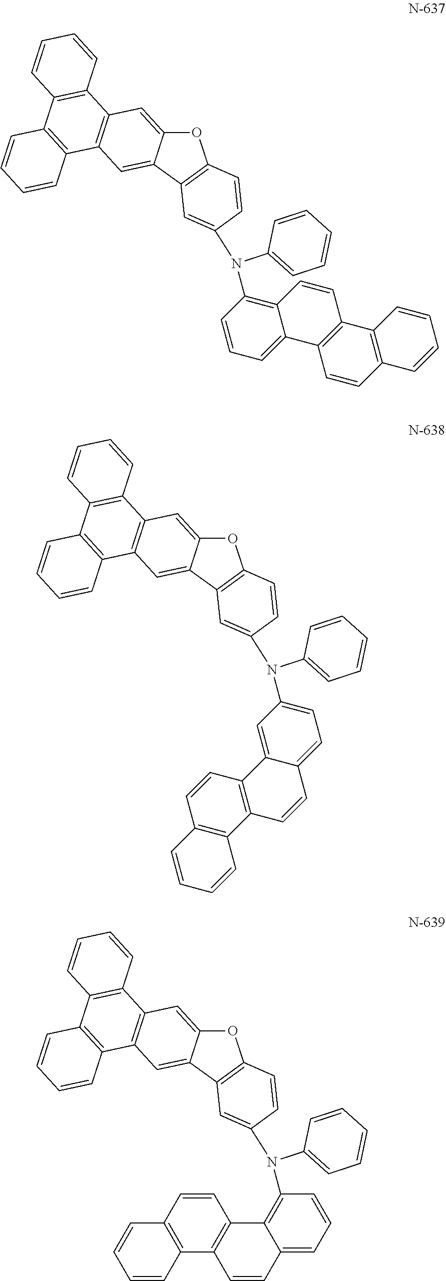 [SMILES: c1ccc(N(c2ccc3oc4cc5c6ccccc6c6ccccc6c5cc4c3c2)c2cccc3c2ccc2c4ccccc4ccc32)cc1 ; c1ccc(N(c2ccc3ccc4c5ccccc5ccc4c3c2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2cccc3ccc4c5ccccc5ccc4c23)cc1]

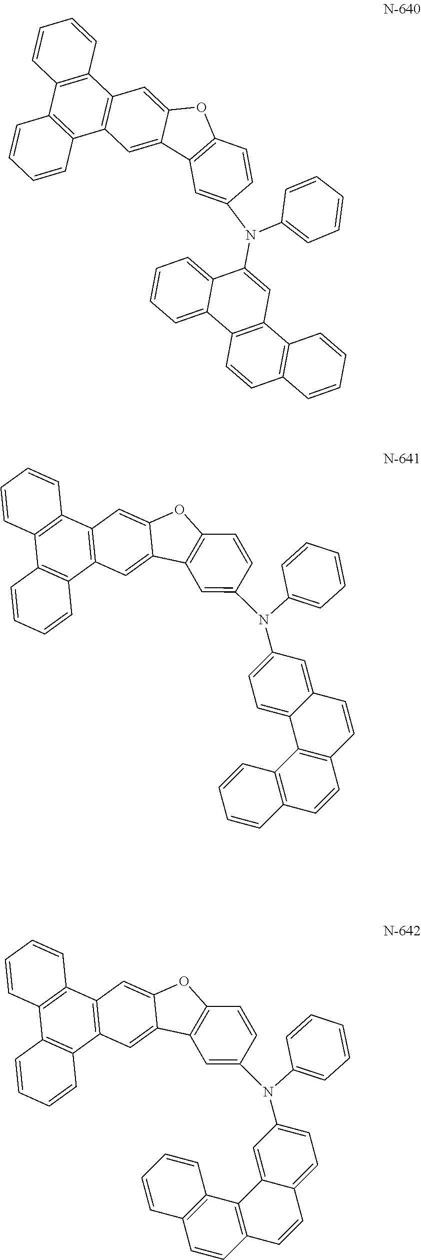 [SMILES: c1ccc(N(c2ccc3oc4cc5c6ccccc6c6ccccc6c5cc4c3c2)c2cc3c4ccccc4ccc3c3ccccc23)cc1 ; c1ccc(N(c2ccc3c(ccc4ccc5ccccc5c43)c2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2ccc3ccc4ccc5ccccc5c4c3c2)cc1]

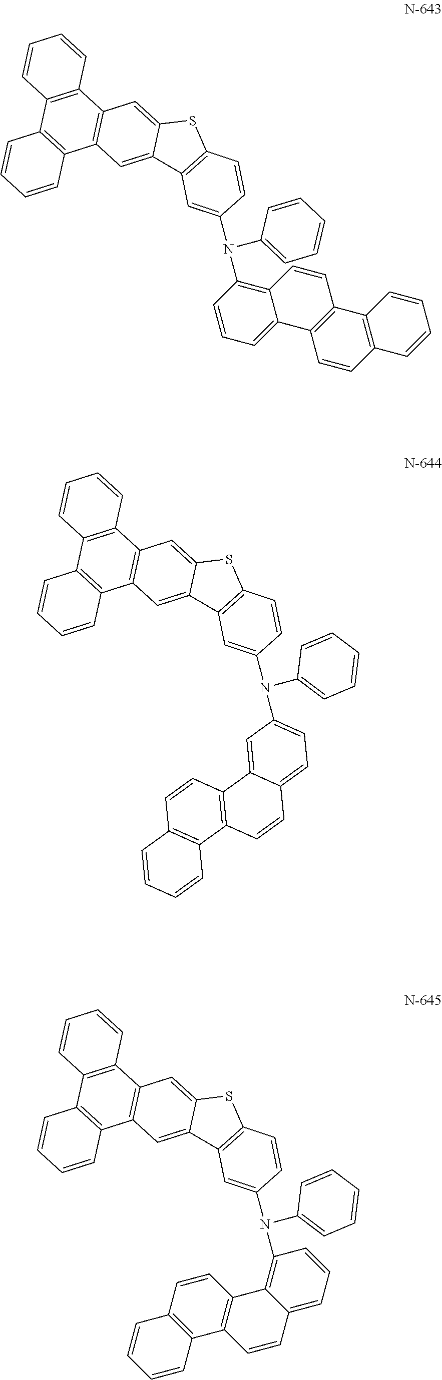 [SMILES: c1ccc(N(c2ccc3sc4cc5c6ccccc6c6ccccc6c5cc4c3c2)c2cccc3c2ccc2c4ccccc4ccc32)cc1 ; c1ccc(N(c2ccc3ccc4c5ccccc5ccc4c3c2)c2ccc3sc4cc5c6ccccc6c6ccccc6c5cc4c3c2)cc1 ; c1ccc(N(c2ccc3sc4cc5c6ccccc6c6ccccc6c5cc4c3c2)c2cccc3ccc4c5ccccc5ccc4c23)cc1]

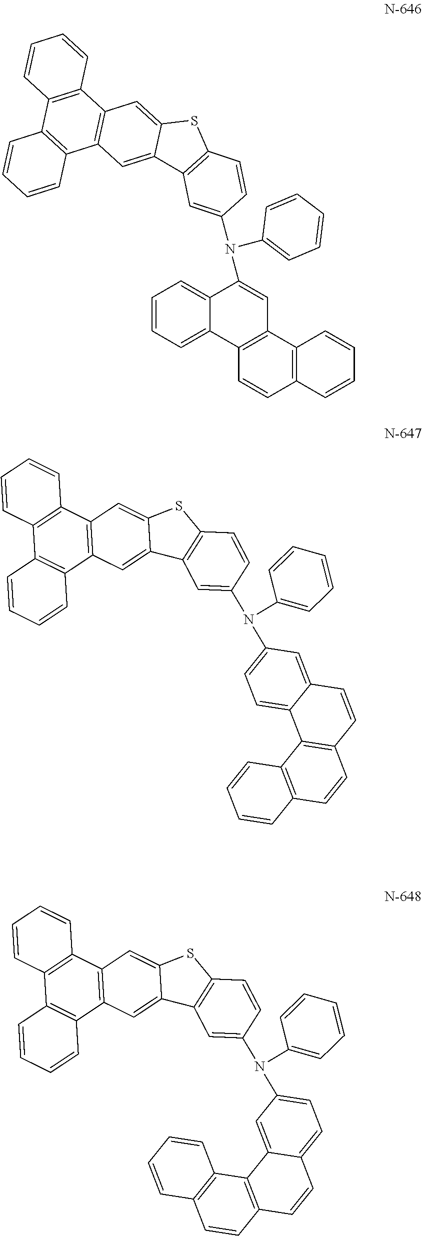 [SMILES: c1ccc(N(c2ccc3sc4cc5c6ccccc6c6ccccc6c5cc4c3c2)c2cc3c4ccccc4ccc3c3ccccc23)cc1 ; c1ccc(N(c2ccc3c(ccc4ccc5ccccc5c43)c2)c2ccc3sc4cc5c6ccccc6c6ccccc6c5cc4c3c2)cc1 ; c1ccc(N(c2ccc3sc4cc5c6ccccc6c6ccccc6c5cc4c3c2)c2ccc3ccc4ccc5ccccc5c4c3c2)cc1]

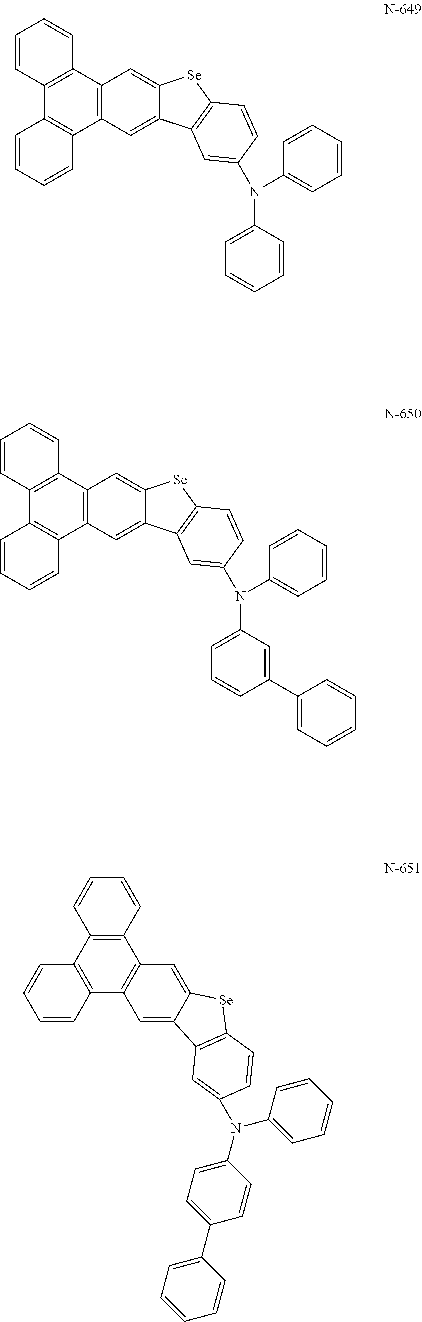 [SMILES: c1ccc(N(c2ccccc2)c2ccc3[se]c4cc5c6ccccc6c6ccccc6c5cc4c3c2)cc1 ; c1ccc(-c2cccc(N(c3ccccc3)c3ccc4[se]c5cc6c7ccccc7c7ccccc7c6cc5c4c3)c2)cc1 ; c1ccc(-c2ccc(N(c3ccccc3)c3ccc4[se]c5cc6c7ccccc7c7ccccc7c6cc5c4c3)cc2)cc1]

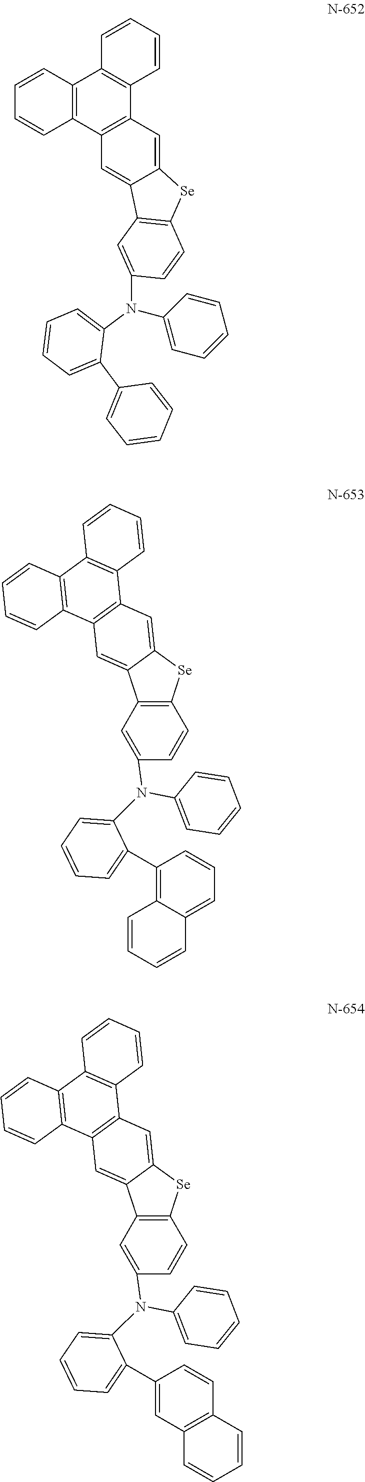 [SMILES: c1ccc(-c2ccccc2N(c2ccccc2)c2ccc3[se]c4cc5c6ccccc6c6ccccc6c5cc4c3c2)cc1 ; c1ccc(N(c2ccc3[se]c4cc5c6ccccc6c6ccccc6c5cc4c3c2)c2ccccc2-c2cccc3ccccc23)cc1 ; c1ccc(N(c2ccc3[se]c4cc5c6ccccc6c6ccccc6c5cc4c3c2)c2ccccc2-c2ccc3ccccc3c2)cc1]

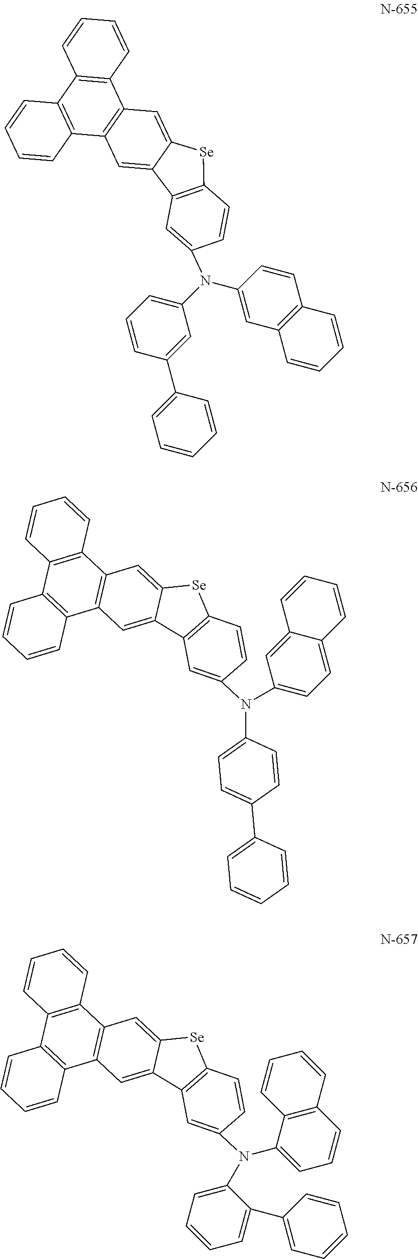 [SMILES: c1ccc(-c2cccc(N(c3ccc4ccccc4c3)c3ccc4[se]c5cc6c7ccccc7c7ccccc7c6cc5c4c3)c2)cc1 ; c1ccc(-c2ccc(N(c3ccc4ccccc4c3)c3ccc4[se]c5cc6c7ccccc7c7ccccc7c6cc5c4c3)cc2)cc1 ; c1ccc(-c2ccccc2N(c2ccc3[se]c4cc5c6ccccc6c6ccccc6c5cc4c3c2)c2cccc3ccccc23)cc1]

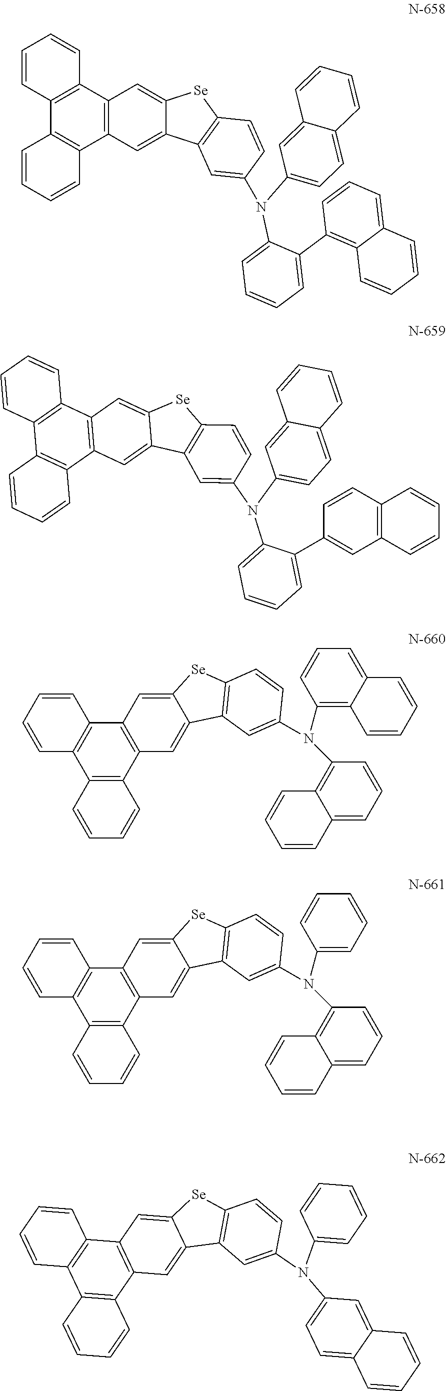

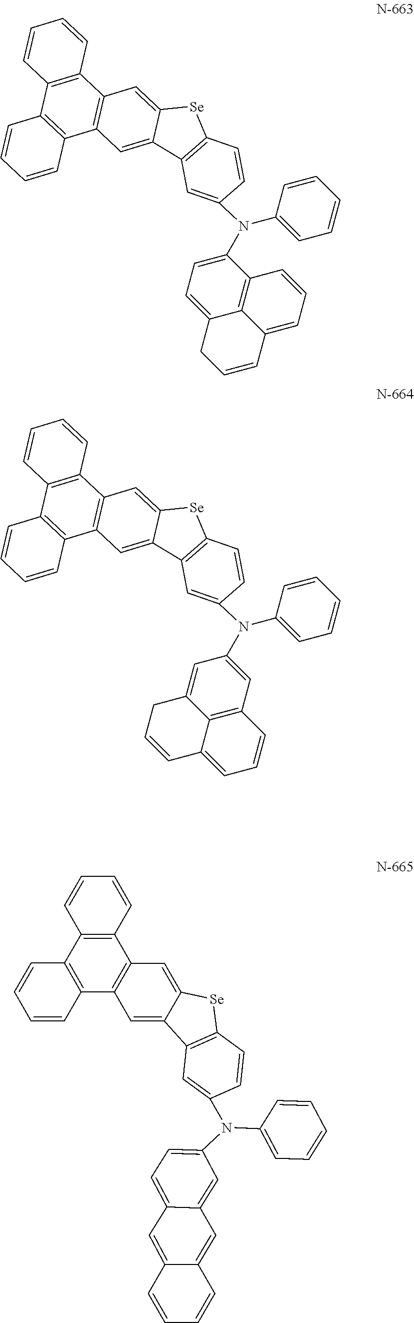 [SMILES: C1=Cc2cccc3c(N(c4ccccc4)c4ccc5[se]c6cc7c8ccccc8c8ccccc8c7cc6c5c4)ccc(c23)C1 ; C1=Cc2cccc3cc(N(c4ccccc4)c4ccc5[se]c6cc7c8ccccc8c8ccccc8c7cc6c5c4)cc(c23)C1 ; c1ccc(N(c2ccc3cc4ccccc4cc3c2)c2ccc3[se]c4cc5c6ccccc6c6ccccc6c5cc4c3c2)cc1]

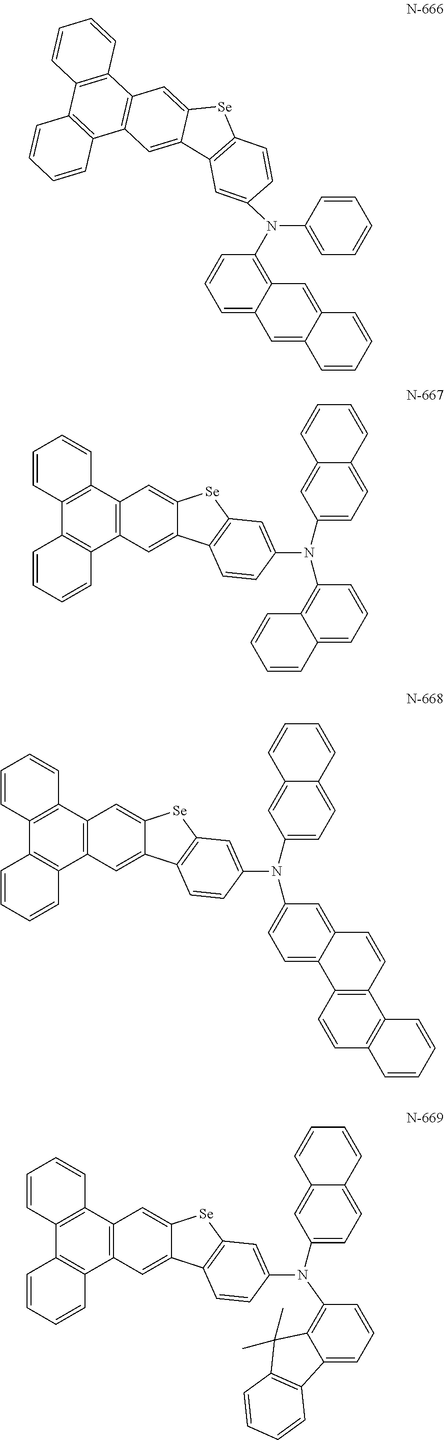

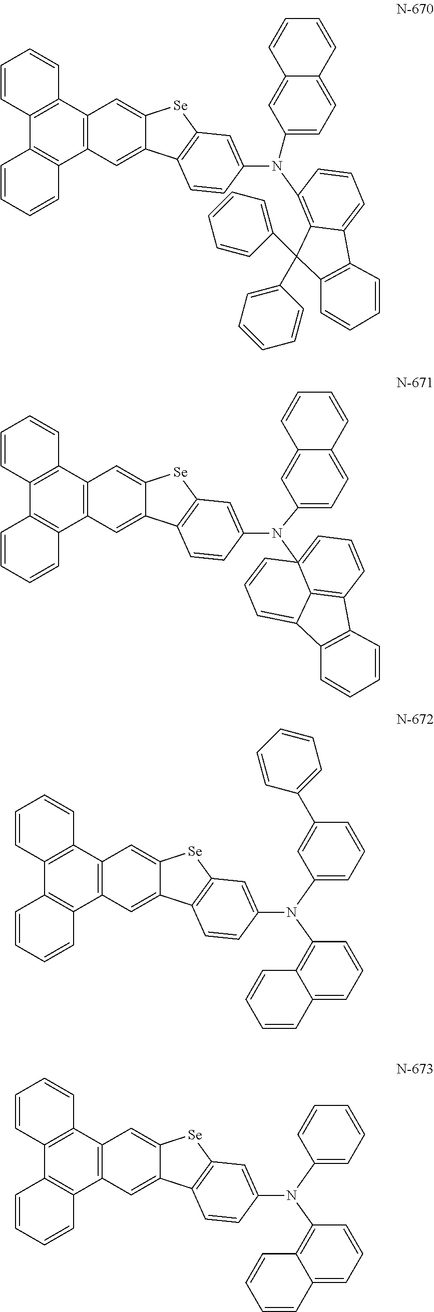

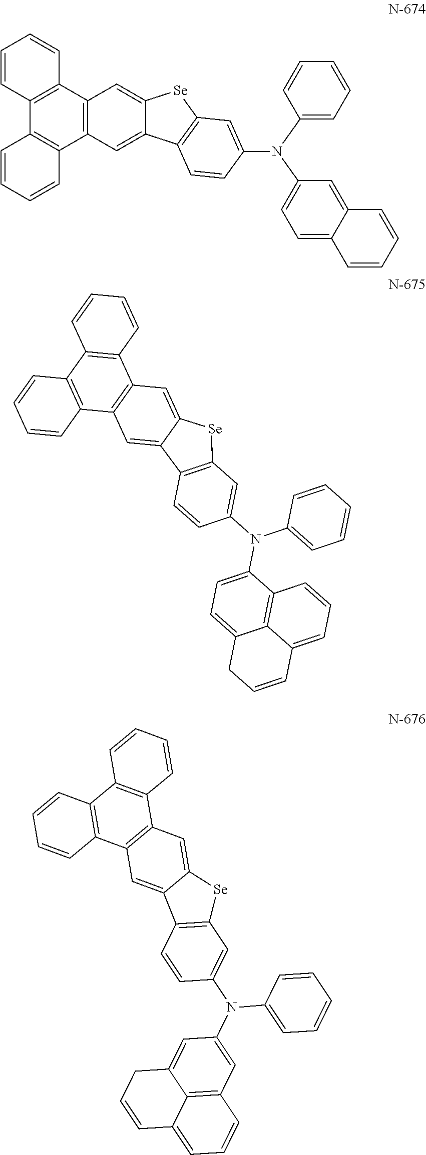 [SMILES: c1ccc(N(c2ccc3ccccc3c2)c2ccc3c(c2)[se]c2cc4c5ccccc5c5ccccc5c4cc23)cc1 ; C1=Cc2cccc3c(N(c4ccccc4)c4ccc5c(c4)[se]c4cc6c7ccccc7c7ccccc7c6cc45)ccc(c23)C1 ; C1=Cc2cccc3cc(N(c4ccccc4)c4ccc5c(c4)[se]c4cc6c7ccccc7c7ccccc7c6cc45)cc(c23)C1]

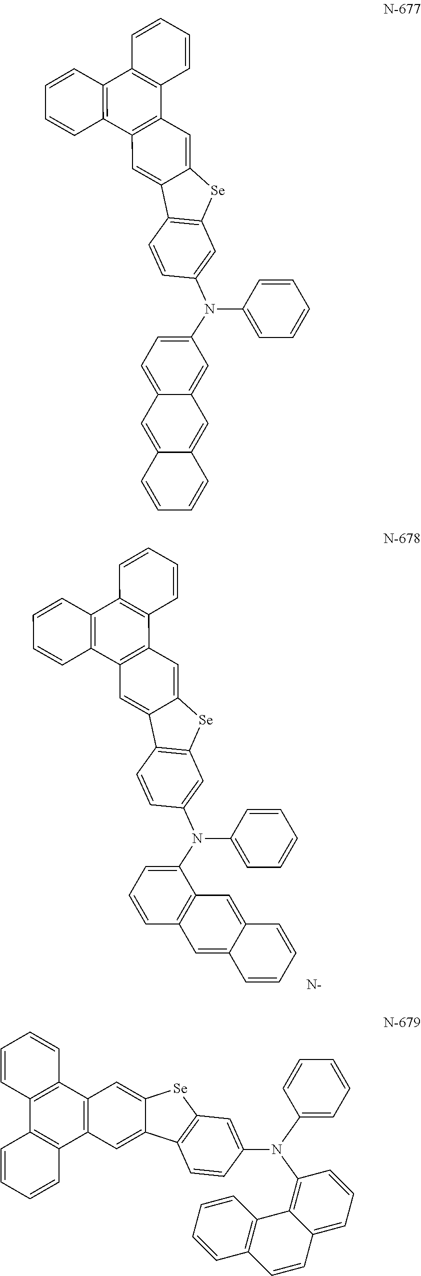

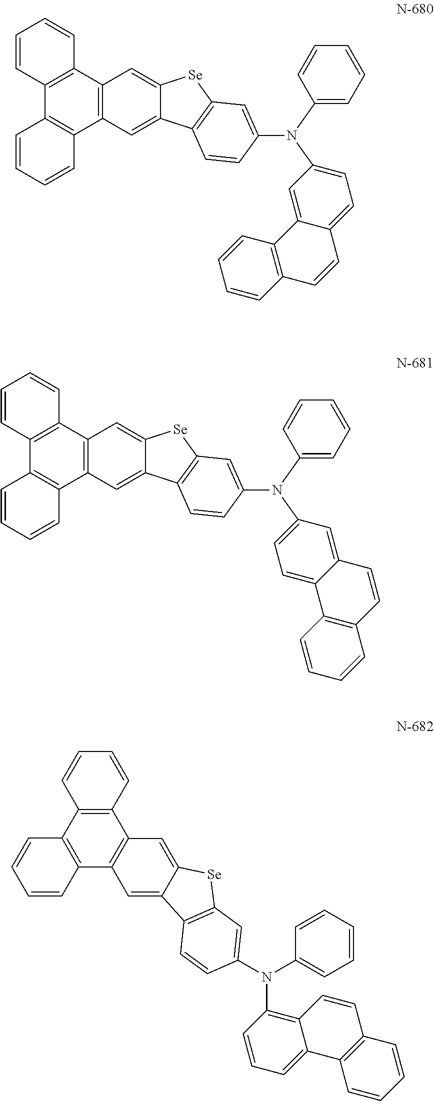 [SMILES: c1ccc(N(c2ccc3c(c2)[se]c2cc4c5ccccc5c5ccccc5c4cc23)c2ccc3ccc4ccccc4c3c2)cc1 ; c1ccc(N(c2ccc3c(ccc4ccccc43)c2)c2ccc3c(c2)[se]c2cc4c5ccccc5c5ccccc5c4cc23)cc1 ; c1ccc(N(c2ccc3c(c2)[se]c2cc4c5ccccc5c5ccccc5c4cc23)c2cccc3c2ccc2ccccc23)cc1]

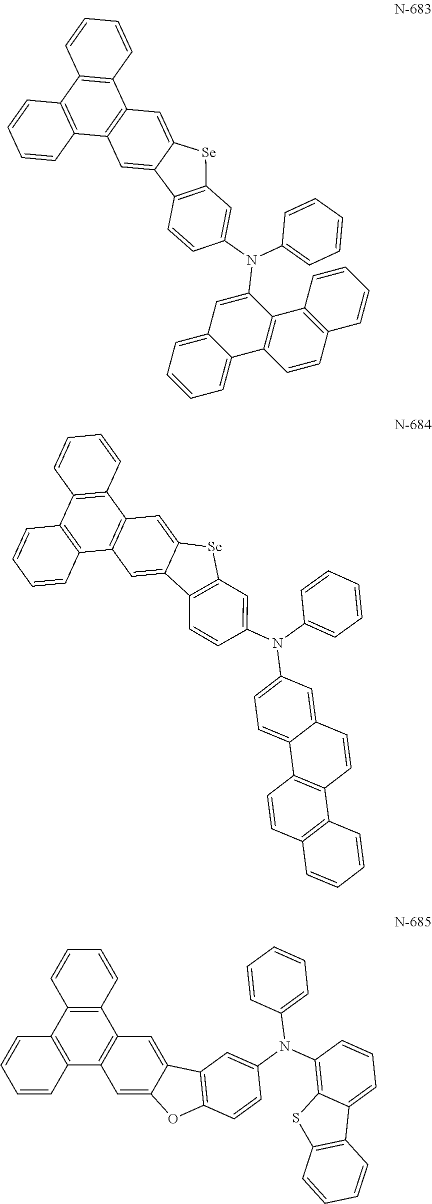 [SMILES: c1ccc(N(c2ccc3c(c2)[se]c2cc4c5ccccc5c5ccccc5c4cc23)c2cc3ccccc3c3ccc4ccccc4c23)cc1 ; c1ccc(N(c2ccc3c(ccc4c5ccccc5ccc34)c2)c2ccc3c(c2)[se]c2cc4c5ccccc5c5ccccc5c4cc23)cc1 ; c1ccc(N(c2ccc3oc4cc5c6ccccc6c6ccccc6c5cc4c3c2)c2cccc3c2sc2ccccc23)cc1]

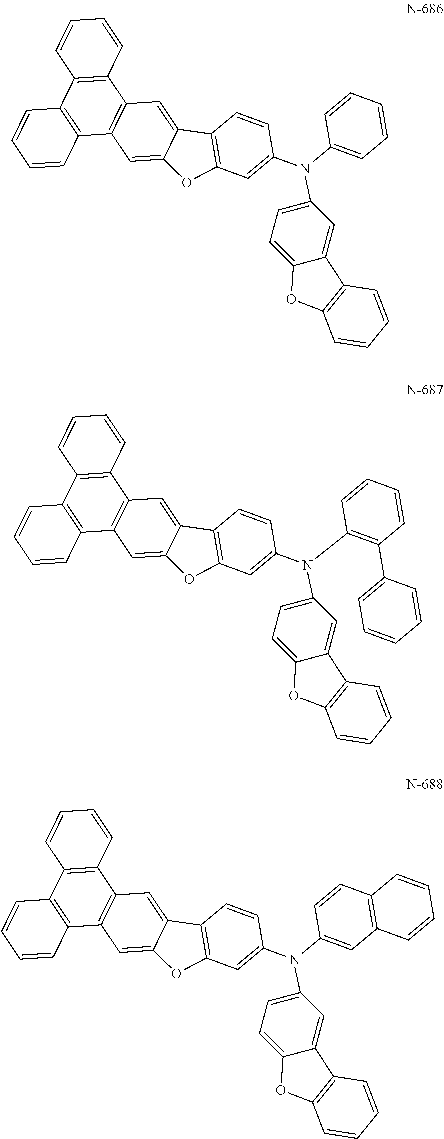 [SMILES: c1ccc(N(c2ccc3c(c2)oc2cc4c5ccccc5c5ccccc5c4cc23)c2ccc3oc4ccccc4c3c2)cc1 ; c1ccc(-c2ccccc2N(c2ccc3c(c2)oc2cc4c5ccccc5c5ccccc5c4cc23)c2ccc3oc4ccccc4c3c2)cc1 ; c1ccc2cc(N(c3ccc4c(c3)oc3cc5c6ccccc6c6ccccc6c5cc34)c3ccc4oc5ccccc5c4c3)ccc2c1]

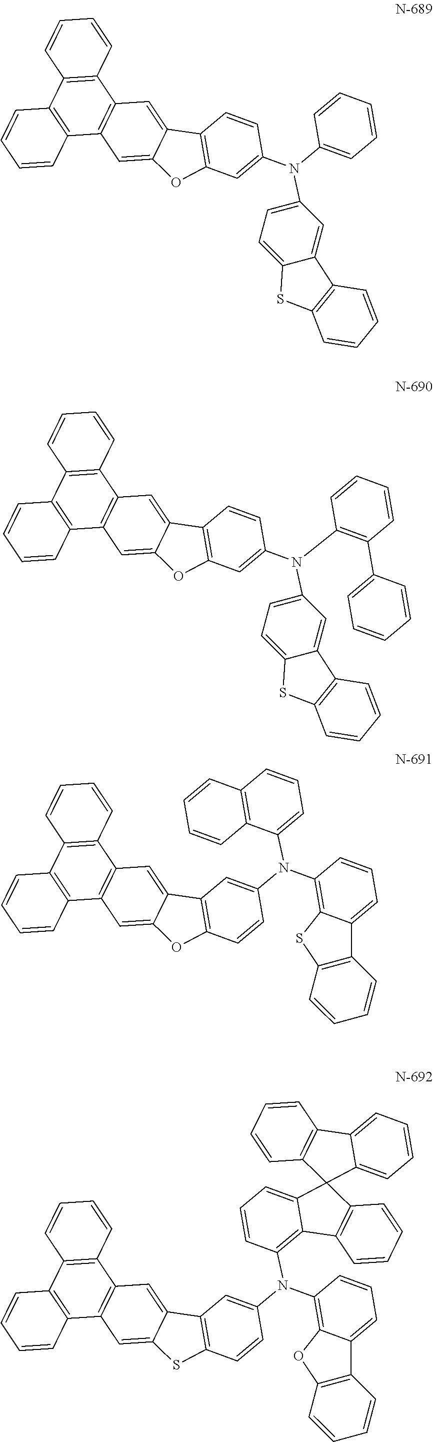 [SMILES: c1ccc(N(c2ccc3c(c2)oc2cc4c5ccccc5c5ccccc5c4cc23)c2ccc3sc4ccccc4c3c2)cc1 ; c1ccc(-c2ccccc2N(c2ccc3c(c2)oc2cc4c5ccccc5c5ccccc5c4cc23)c2ccc3sc4ccccc4c3c2)cc1 ; c1ccc2c(N(c3ccc4oc5cc6c7ccccc7c7ccccc7c6cc5c4c3)c3cccc4c3sc3ccccc34)cccc2c1 ; c1ccc2c(c1)-c1ccccc1C21c2ccccc2-c2c(N(c3ccc4sc5cc6c7ccccc7c7ccccc7c6cc5c4c3)c3cccc4c3oc3ccccc34)cccc21]

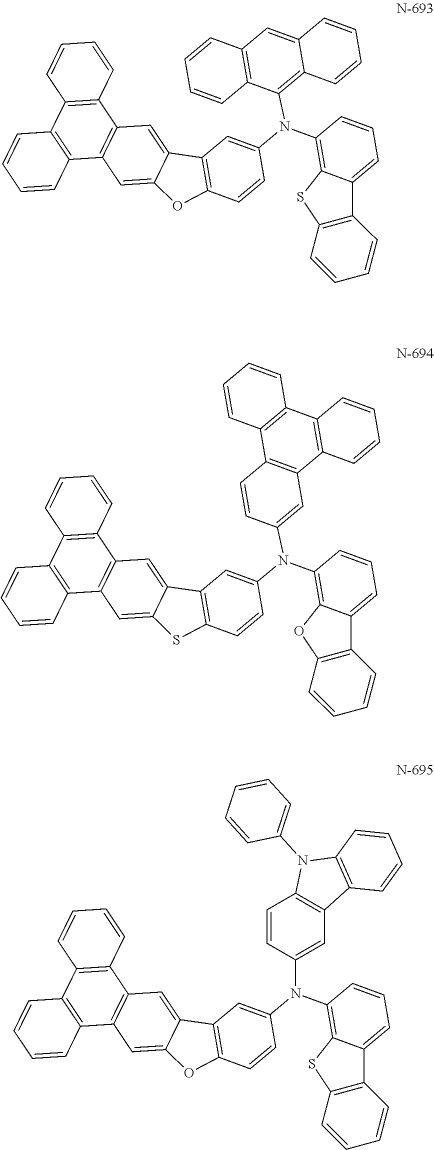 [SMILES: c1ccc2c(N(c3ccc4oc5cc6c7ccccc7c7ccccc7c6cc5c4c3)c3cccc4c3sc3ccccc34)c3ccccc3cc2c1 ; c1ccc2c(c1)oc1c(N(c3ccc4sc5cc6c7ccccc7c7ccccc7c6cc5c4c3)c3ccc4c5ccccc5c5ccccc5c4c3)cccc12 ; c1ccc(-n2c3ccccc3c3cc(N(c4ccc5oc6cc7c8ccccc8c8ccccc8c7cc6c5c4)c4cccc5c4sc4ccccc45)ccc32)cc1]

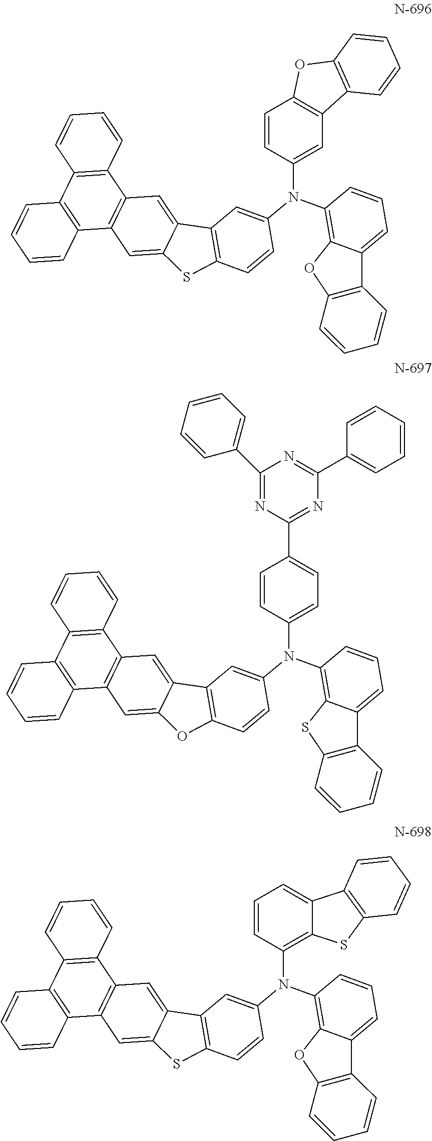 [SMILES: c1ccc2c(c1)oc1ccc(N(c3ccc4sc5cc6c7ccccc7c7ccccc7c6cc5c4c3)c3cccc4c3oc3ccccc34)cc12 ; c1ccc(-c2nc(-c3ccccc3)nc(-c3ccc(N(c4ccc5oc6cc7c8ccccc8c8ccccc8c7cc6c5c4)c4cccc5c4sc4ccccc45)cc3)n2)cc1 ; c1ccc2c(c1)oc1c(N(c3ccc4sc5cc6c7ccccc7c7ccccc7c6cc5c4c3)c3cccc4c3sc3ccccc34)cccc12]

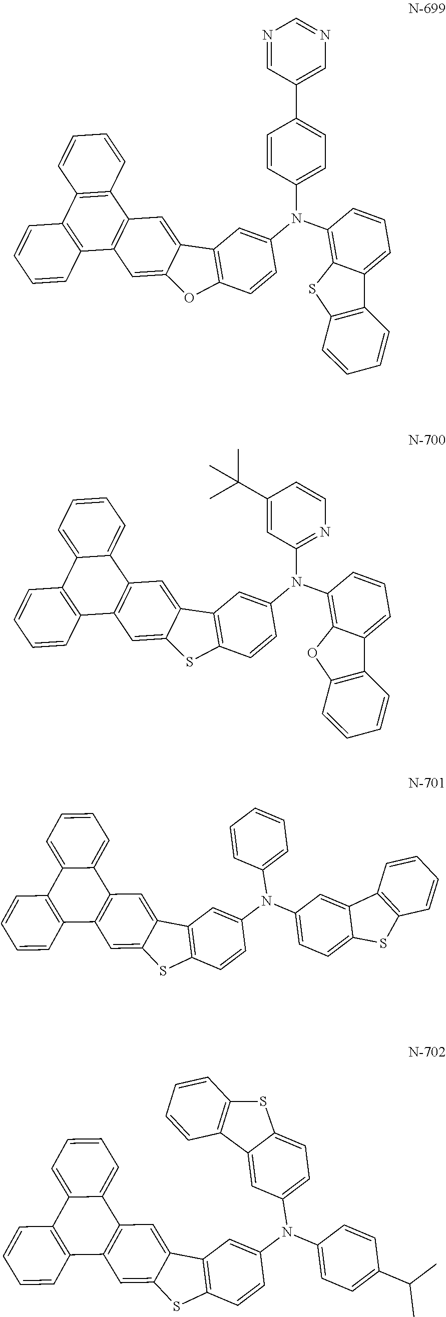 [SMILES: c1ccc2c(c1)sc1c(N(c3ccc(-c4cncnc4)cc3)c3ccc4oc5cc6c7ccccc7c7ccccc7c6cc5c4c3)cccc12 ; CC(C)(C)c1ccnc(N(c2ccc3sc4cc5c6ccccc6c6ccccc6c5cc4c3c2)c2cccc3c2oc2ccccc23)c1 ; c1ccc(N(c2ccc3sc4ccccc4c3c2)c2ccc3sc4cc5c6ccccc6c6ccccc6c5cc4c3c2)cc1 ; CC(C)c1ccc(N(c2ccc3sc4ccccc4c3c2)c2ccc3sc4cc5c6ccccc6c6ccccc6c5cc4c3c2)cc1]

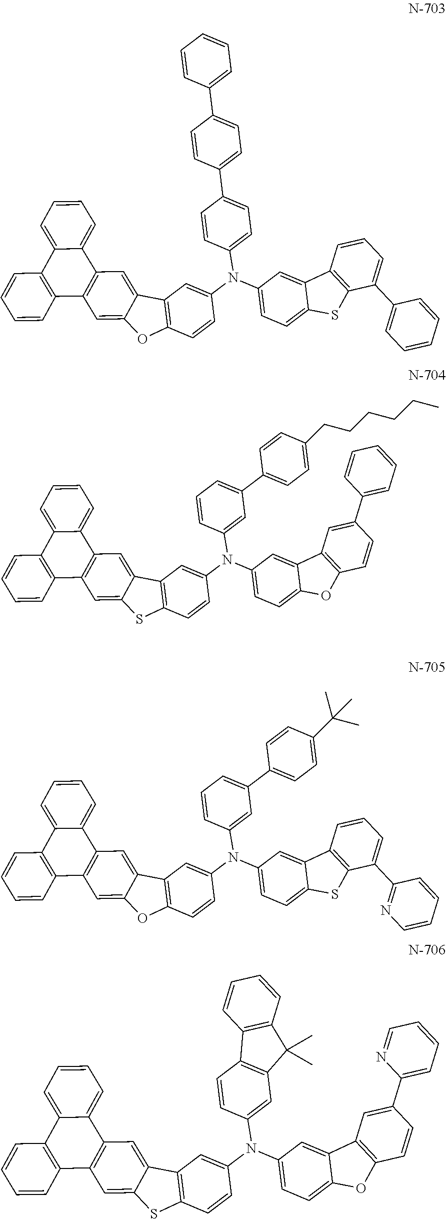 [SMILES: c1ccc(-c2ccc(-c3ccc(N(c4ccc5oc6cc7c8ccccc8c8ccccc8c7cc6c5c4)c4ccc5sc6c(-c7ccccc7)cccc6c5c4)cc3)cc2)cc1 ; CCCCCCc1ccc(-c2cccc(N(c3ccc4oc5ccc(-c6ccccc6)cc5c4c3)c3ccc4sc5cc6c7ccccc7c7ccccc7c6cc5c4c3)c2)cc1 ; CC(C)(C)c1ccc(-c2cccc(N(c3ccc4oc5cc6c7ccccc7c7ccccc7c6cc5c4c3)c3ccc4sc5c(-c6ccccn6)cccc5c4c3)c2)cc1 ; CC1(C)c2ccccc2-c2ccc(N(c3ccc4oc5ccc(-c6ccccn6)cc5c4c3)c3ccc4sc5cc6c7ccccc7c7ccccc7c6cc5c4c3)cc21]

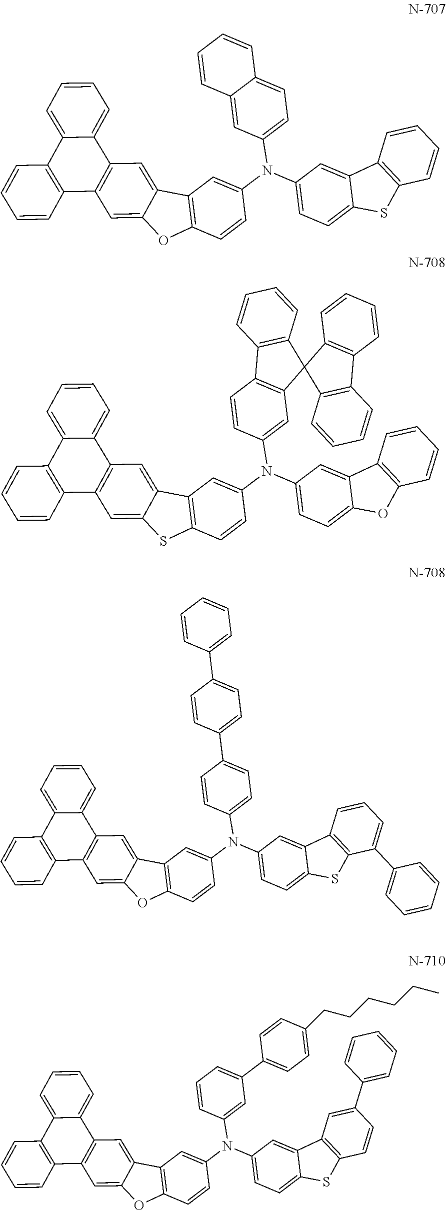 [SMILES: c1ccc2cc(N(c3ccc4oc5cc6c7ccccc7c7ccccc7c6cc5c4c3)c3ccc4sc5ccccc5c4c3)ccc2c1 ; c1ccc2c(c1)-c1ccccc1C21c2ccccc2-c2ccc(N(c3ccc4oc5ccccc5c4c3)c3ccc4sc5cc6c7ccccc7c7ccccc7c6cc5c4c3)cc21 ; c1ccc(-c2ccc(-c3ccc(N(c4ccc5oc6cc7c8ccccc8c8ccccc8c7cc6c5c4)c4ccc5sc6c(-c7ccccc7)cccc6c5c4)cc3)cc2)cc1 ; CCCCCCc1ccc(-c2cccc(N(c3ccc4oc5cc6c7ccccc7c7ccccc7c6cc5c4c3)c3ccc4sc5ccc(-c6ccccc6)cc5c4c3)c2)cc1]

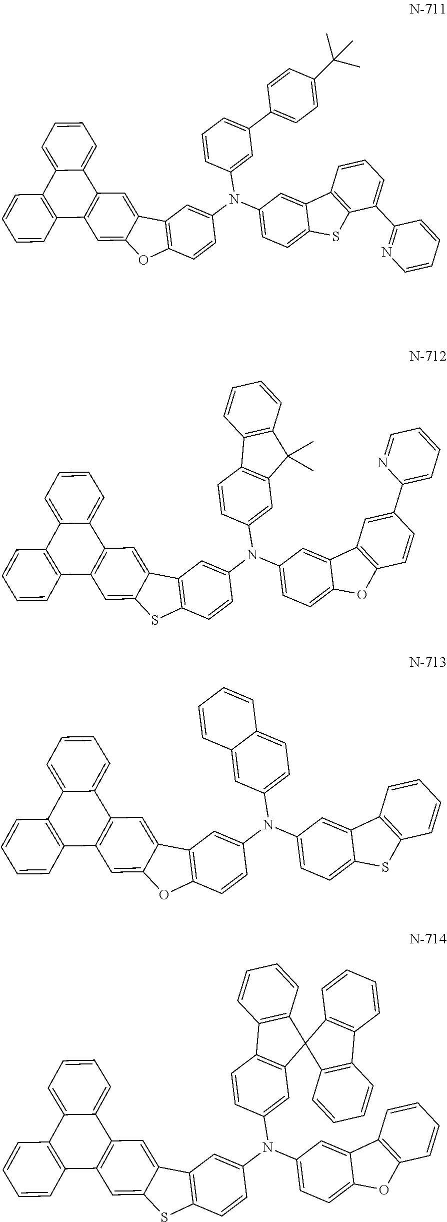 [SMILES: CC(C)(C)c1ccc(-c2cccc(N(c3ccc4oc5cc6c7ccccc7c7ccccc7c6cc5c4c3)c3ccc4sc5c(-c6ccccn6)cccc5c4c3)c2)cc1 ; CC1(C)c2ccccc2-c2ccc(N(c3ccc4oc5ccc(-c6ccccn6)cc5c4c3)c3ccc4sc5cc6c7ccccc7c7ccccc7c6cc5c4c3)cc21 ; c1ccc2cc(N(c3ccc4oc5cc6c7ccccc7c7ccccc7c6cc5c4c3)c3ccc4sc5ccccc5c4c3)ccc2c1 ; c1ccc2c(c1)-c1ccccc1C21c2ccccc2-c2ccc(N(c3ccc4oc5ccccc5c4c3)c3ccc4sc5cc6c7ccccc7c7ccccc7c6cc5c4c3)cc21]

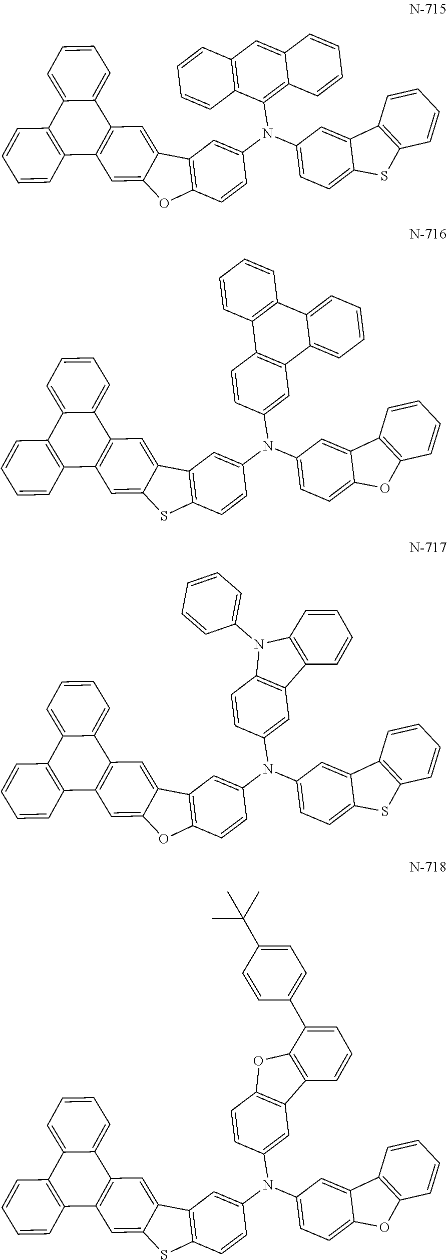 [SMILES: c1ccc2c(N(c3ccc4oc5cc6c7ccccc7c7ccccc7c6cc5c4c3)c3ccc4sc5ccccc5c4c3)c3ccccc3cc2c1 ; c1ccc2c(c1)oc1ccc(N(c3ccc4sc5cc6c7ccccc7c7ccccc7c6cc5c4c3)c3ccc4c5ccccc5c5ccccc5c4c3)cc12 ; c1ccc(-n2c3ccccc3c3cc(N(c4ccc5oc6cc7c8ccccc8c8ccccc8c7cc6c5c4)c4ccc5sc6ccccc6c5c4)ccc32)cc1 ; CC(C)(C)c1ccc(-c2cccc3c2oc2ccc(N(c4ccc5oc6ccccc6c5c4)c4ccc5sc6cc7c8ccccc8c8ccccc8c7cc6c5c4)cc23)cc1]

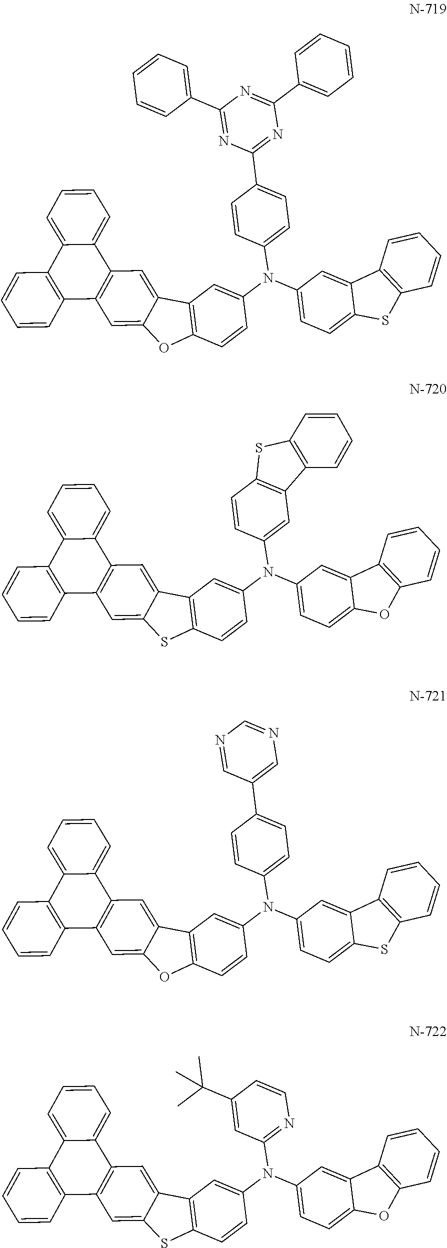 [SMILES: c1ccc(-c2nc(-c3ccccc3)nc(-c3ccc(N(c4ccc5oc6cc7c8ccccc8c8ccccc8c7cc6c5c4)c4ccc5sc6ccccc6c5c4)cc3)n2)cc1 ; c1ccc2c(c1)oc1ccc(N(c3ccc4sc5ccccc5c4c3)c3ccc4sc5cc6c7ccccc7c7ccccc7c6cc5c4c3)cc12 ; c1ccc2c(c1)sc1ccc(N(c3ccc(-c4cncnc4)cc3)c3ccc4oc5cc6c7ccccc7c7ccccc7c6cc5c4c3)cc12 ; CC(C)(C)c1ccnc(N(c2ccc3oc4ccccc4c3c2)c2ccc3sc4cc5c6ccccc6c6ccccc6c5cc4c3c2)c1]

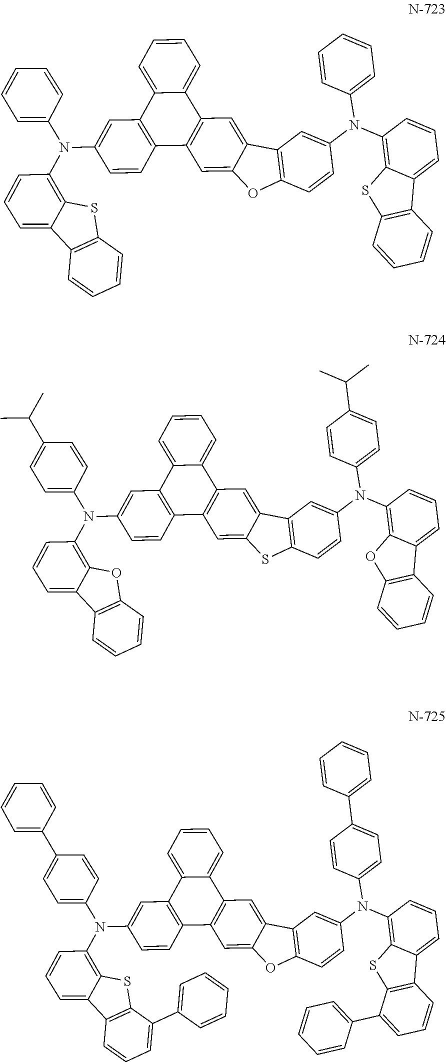 [SMILES: c1ccc(N(c2ccc3oc4cc5c6ccc(N(c7ccccc7)c7cccc8c7sc7ccccc78)cc6c6ccccc6c5cc4c3c2)c2cccc3c2sc2ccccc23)cc1 ; CC(C)c1ccc(N(c2ccc3sc4cc5c6ccc(N(c7ccc(C(C)C)cc7)c7cccc8c7oc7ccccc78)cc6c6ccccc6c5cc4c3c2)c2cccc3c2oc2ccccc23)cc1 ; c1ccc(-c2ccc(N(c3ccc4oc5cc6c7ccc(N(c8ccc(-c9ccccc9)cc8)c8cccc9c8sc8c(-c%10ccccc%10)cccc89)cc7c7ccccc7c6cc5c4c3)c3cccc4c3sc3c(-c5ccccc5)cccc34)cc2)cc1]

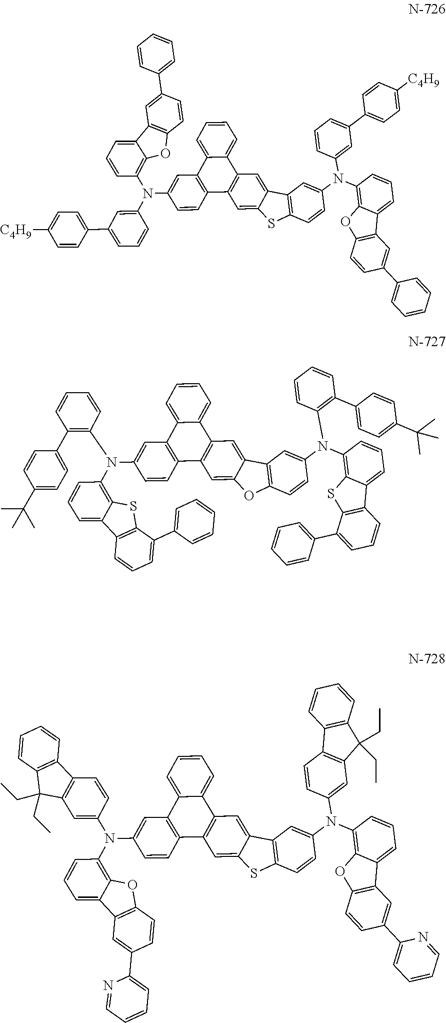

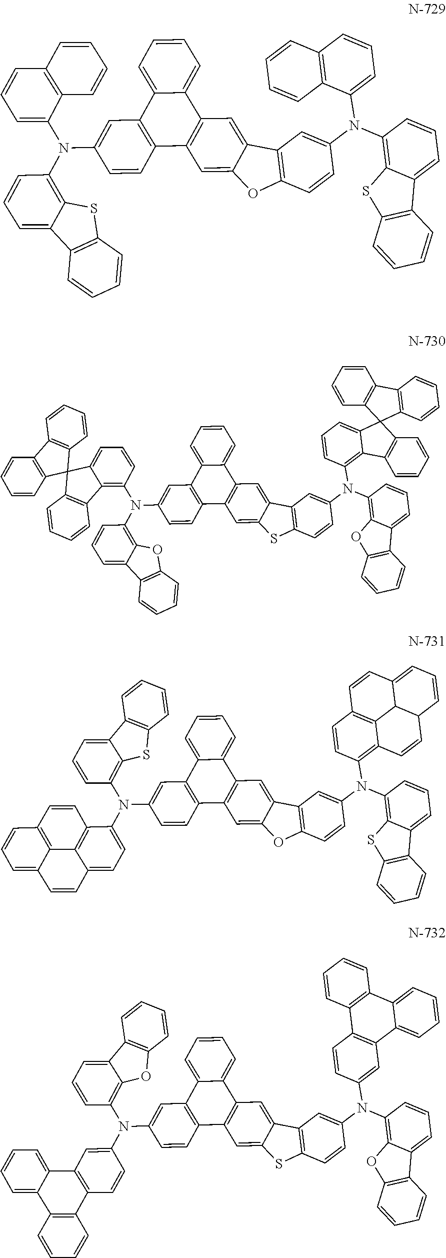

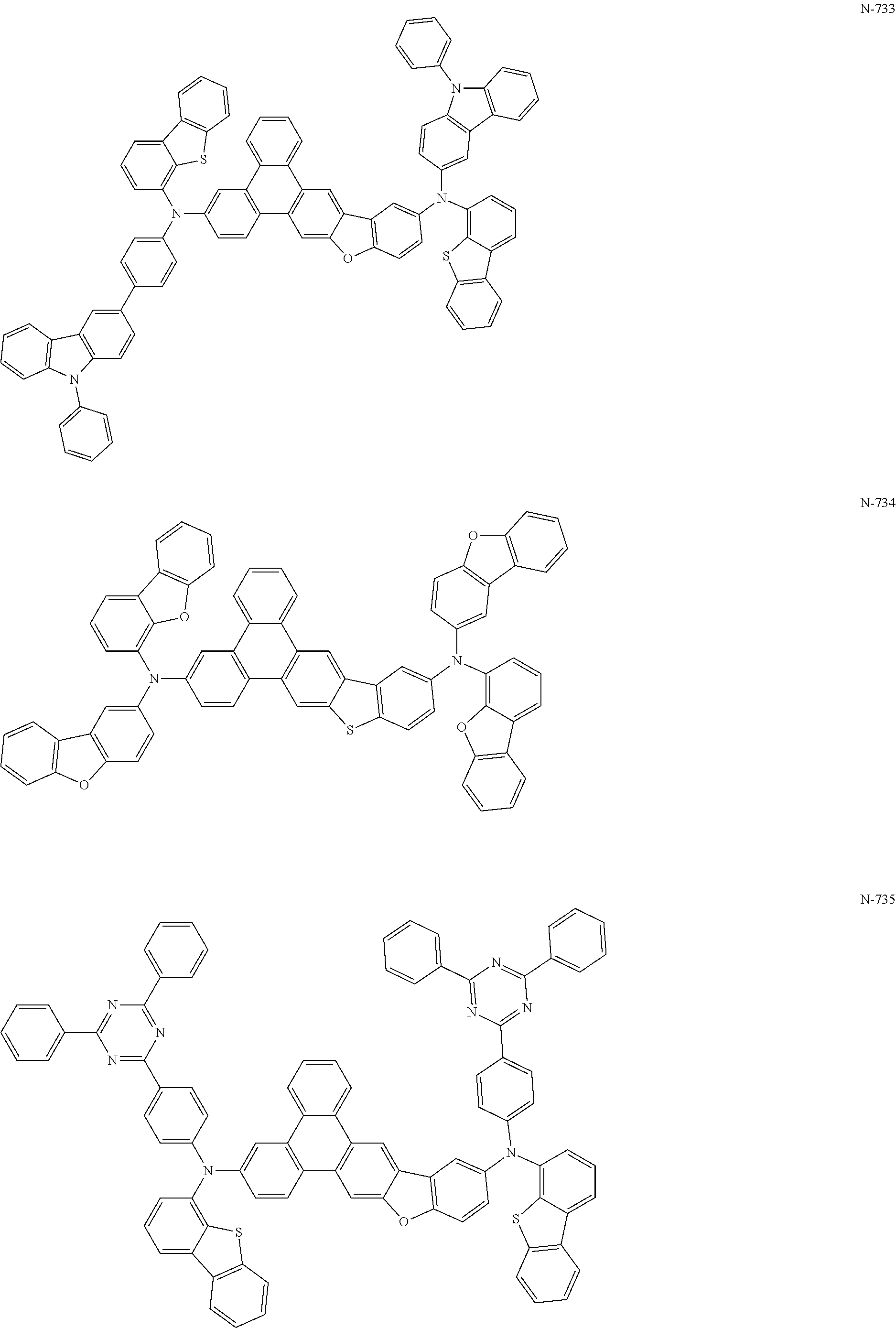 [SMILES: c1ccc(-n2c3ccccc3c3cc(-c4ccc(N(c5ccc6c(c5)c5ccccc5c5cc7c(cc65)oc5ccc(N(c6ccc8c(c6)c6ccccc6n8-c6ccccc6)c6cccc8c6sc6ccccc68)cc57)c5cccc6c5sc5ccccc56)cc4)ccc32)cc1 ; c1ccc2c(c1)oc1ccc(N(c3ccc4sc5cc6c7ccc(N(c8ccc9oc%10ccccc%10c9c8)c8cccc9c8oc8ccccc89)cc7c7ccccc7c6cc5c4c3)c3cccc4c3oc3ccccc34)cc12 ; c1ccc(-c2nc(-c3ccccc3)nc(-c3ccc(N(c4ccc5oc6cc7c8ccc(N(c9ccc(-c%10nc(-c%11ccccc%11)nc(-c%11ccccc%11)n%10)cc9)c9cccc%10c9sc9ccccc9%10)cc8c8ccccc8c7cc6c5c4)c4cccc5c4sc4ccccc45)cc3)n2)cc1]

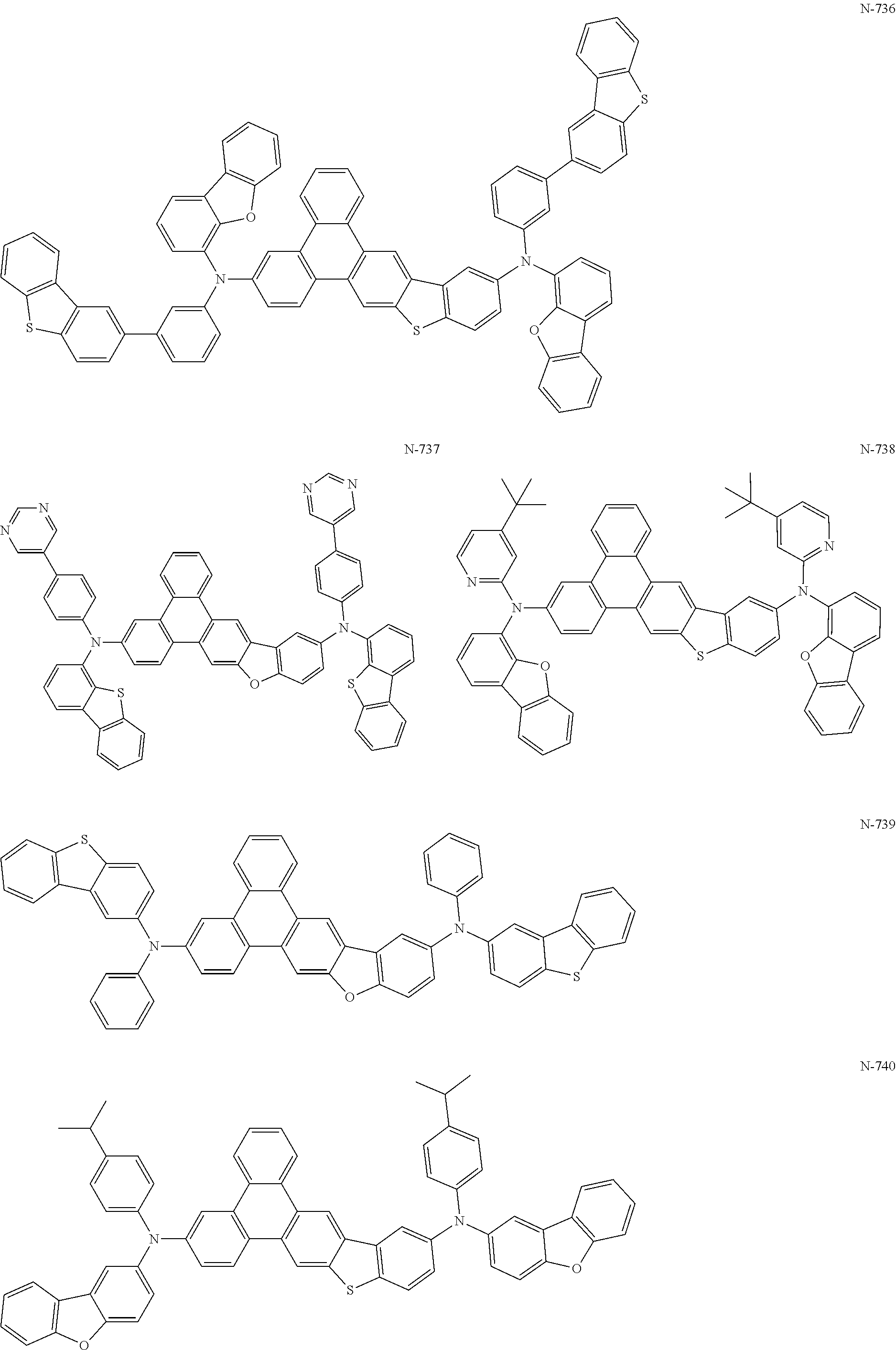

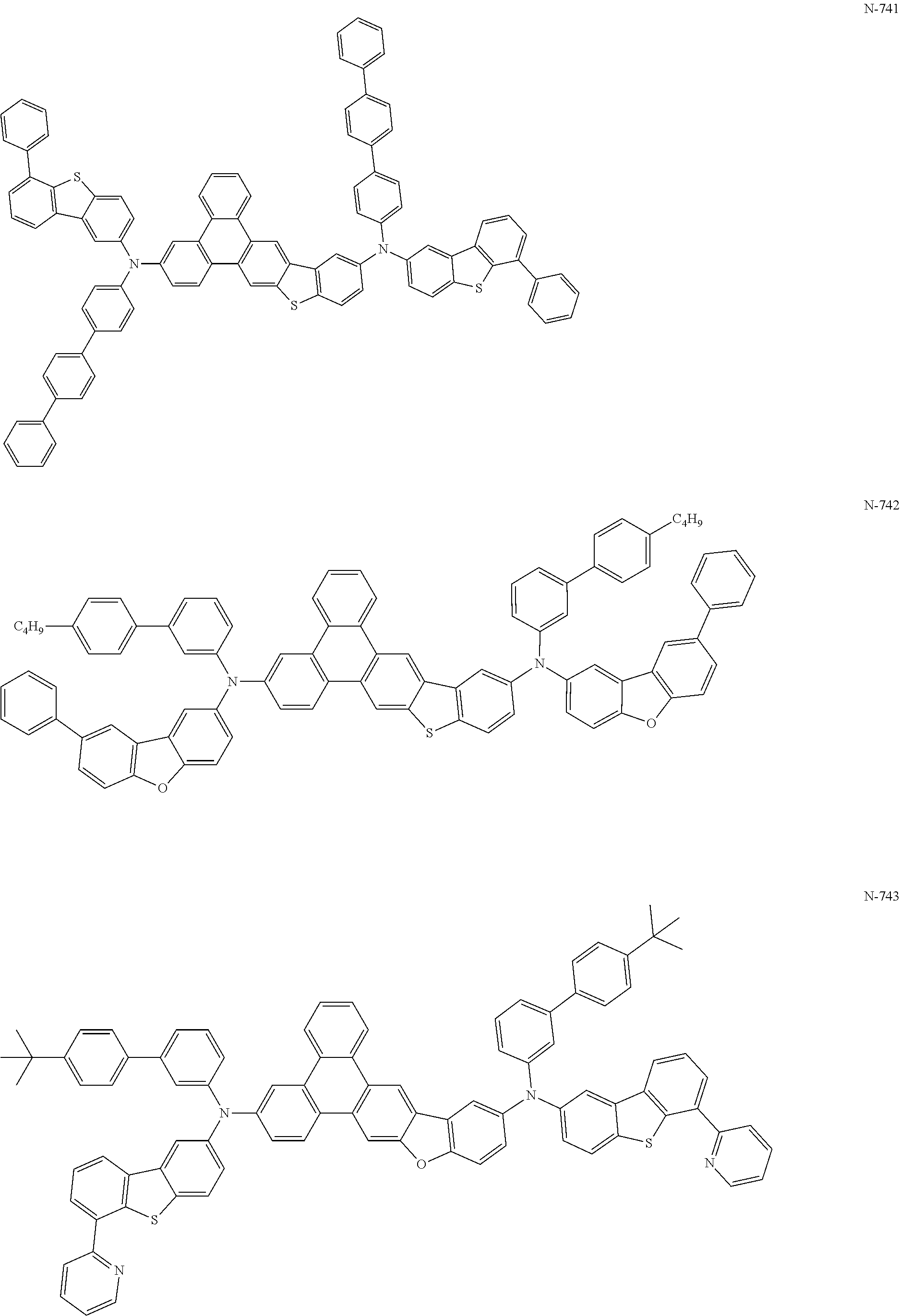

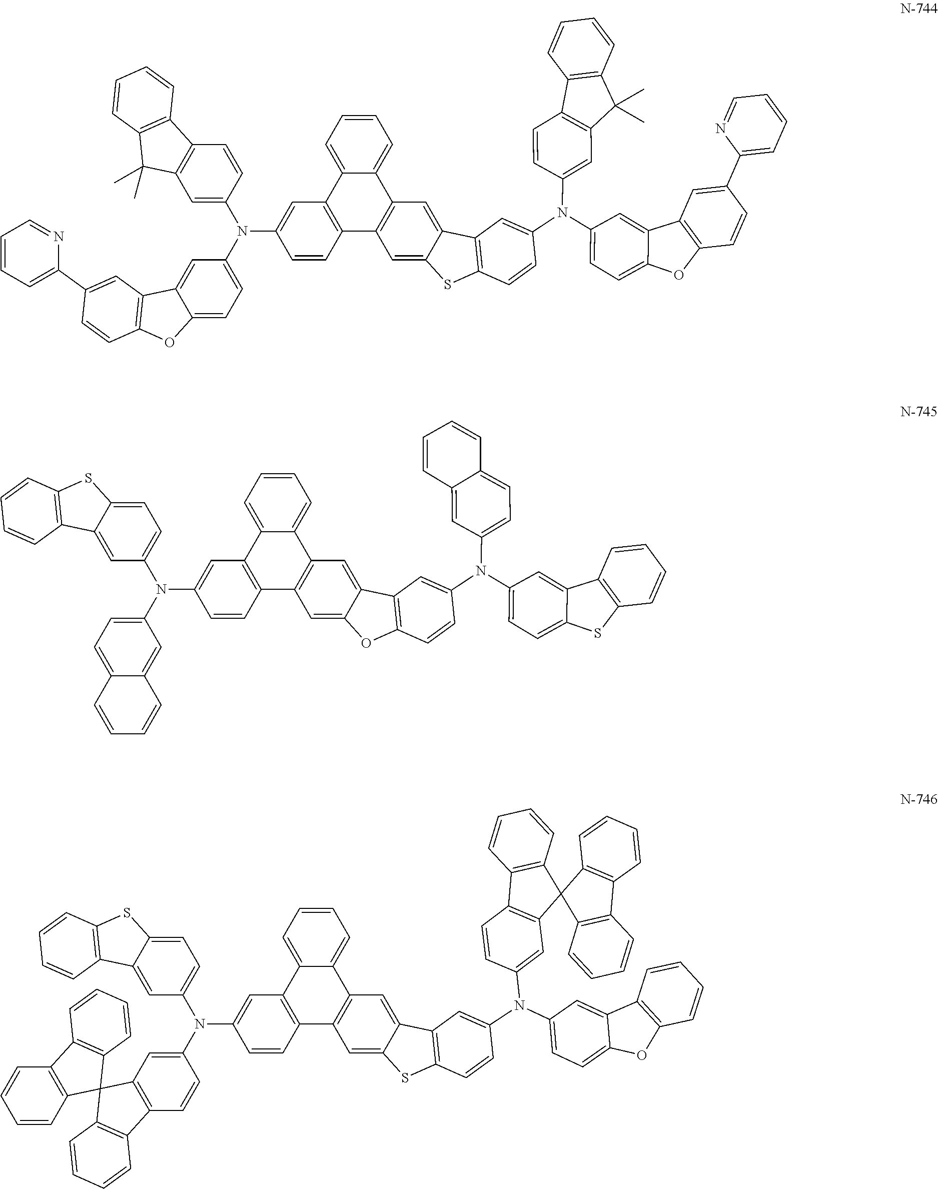 [SMILES: CC1(C)c2ccccc2-c2ccc(N(c3ccc4oc5ccc(-c6ccccn6)cc5c4c3)c3ccc4sc5cc6c7ccc(N(c8ccc9c(c8)C(C)(C)c8ccccc8-9)c8ccc9oc%10ccc(-c%11ccccn%11)cc%10c9c8)cc7c7ccccc7c6cc5c4c3)cc21 ; c1ccc2cc(N(c3ccc4oc5cc6c7ccc(N(c8ccc9ccccc9c8)c8ccc9sc%10ccccc%10c9c8)cc7c7ccccc7c6cc5c4c3)c3ccc4sc5ccccc5c4c3)ccc2c1 ; c1ccc2c(c1)-c1ccccc1C21c2ccccc2-c2ccc(N(c3ccc4oc5ccccc5c4c3)c3ccc4sc5cc6c7ccc(N(c8ccc9c(c8)C8(c%10ccccc%10-c%10ccccc%108)c8ccccc8-9)c8ccc9sc%10ccccc%10c9c8)cc7c7ccccc7c6cc5c4c3)cc21]

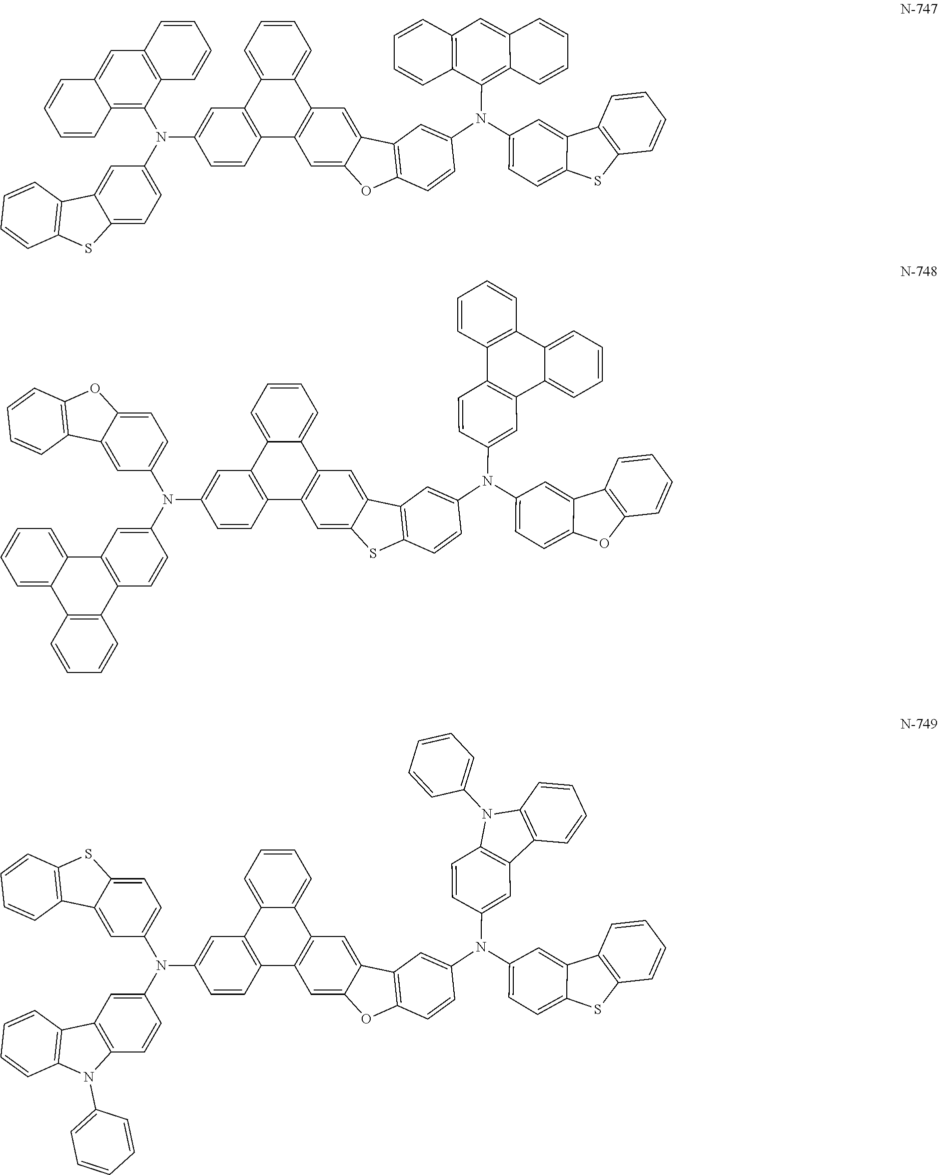 [SMILES: c1ccc2c(N(c3ccc4oc5cc6c7ccc(N(c8ccc9sc%10ccccc%10c9c8)c8c9ccccc9cc9ccccc89)cc7c7ccccc7c6cc5c4c3)c3ccc4sc5ccccc5c4c3)c3ccccc3cc2c1 ; c1ccc2c(c1)oc1ccc(N(c3ccc4sc5cc6c7ccc(N(c8ccc9oc%10ccccc%10c9c8)c8ccc9c%10ccccc%10c%10ccccc%10c9c8)cc7c7ccccc7c6cc5c4c3)c3ccc4c5ccccc5c5ccccc5c4c3)cc12 ; c1ccc(-n2c3ccccc3c3cc(N(c4ccc5oc6cc7c8ccc(N(c9ccc%10sc%11ccccc%11c%10c9)c9ccc%10c(c9)c9ccccc9n%10-c9ccccc9)cc8c8ccccc8c7cc6c5c4)c4ccc5sc6ccccc6c5c4)ccc32)cc1]

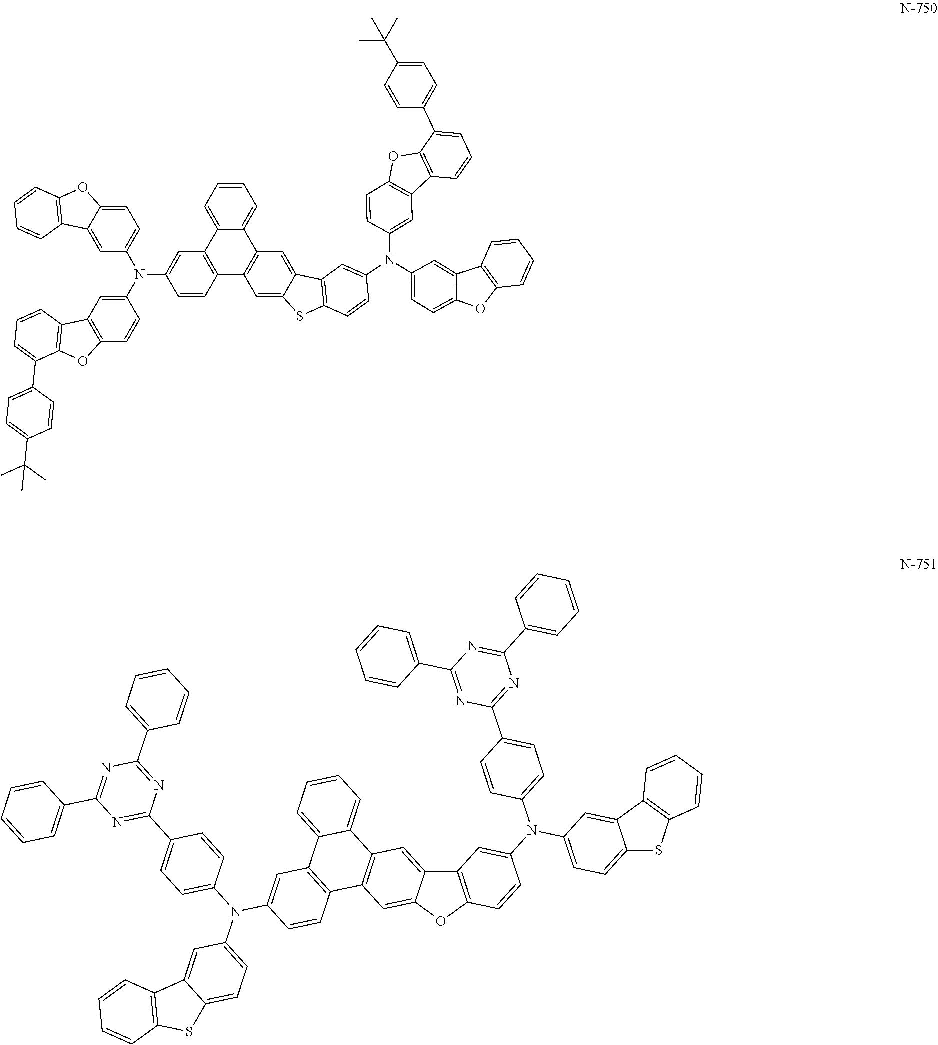 [SMILES: CC(C)(C)c1ccc(-c2cccc3c2oc2ccc(N(c4ccc5oc6ccccc6c5c4)c4ccc5sc6cc7c8ccc(N(c9ccc%10oc%11ccccc%11c%10c9)c9ccc%10oc%11c(-c%12ccc(C(C)(C)C)cc%12)cccc%11c%10c9)cc8c8ccccc8c7cc6c5c4)cc23)cc1 ; c1ccc(-c2nc(-c3ccccc3)nc(-c3ccc(N(c4ccc5oc6cc7c8ccc(N(c9ccc(-c%10nc(-c%11ccccc%11)nc(-c%11ccccc%11)n%10)cc9)c9ccc%10sc%11ccccc%11c%10c9)cc8c8ccccc8c7cc6c5c4)c4ccc5sc6ccccc6c5c4)cc3)n2)cc1]

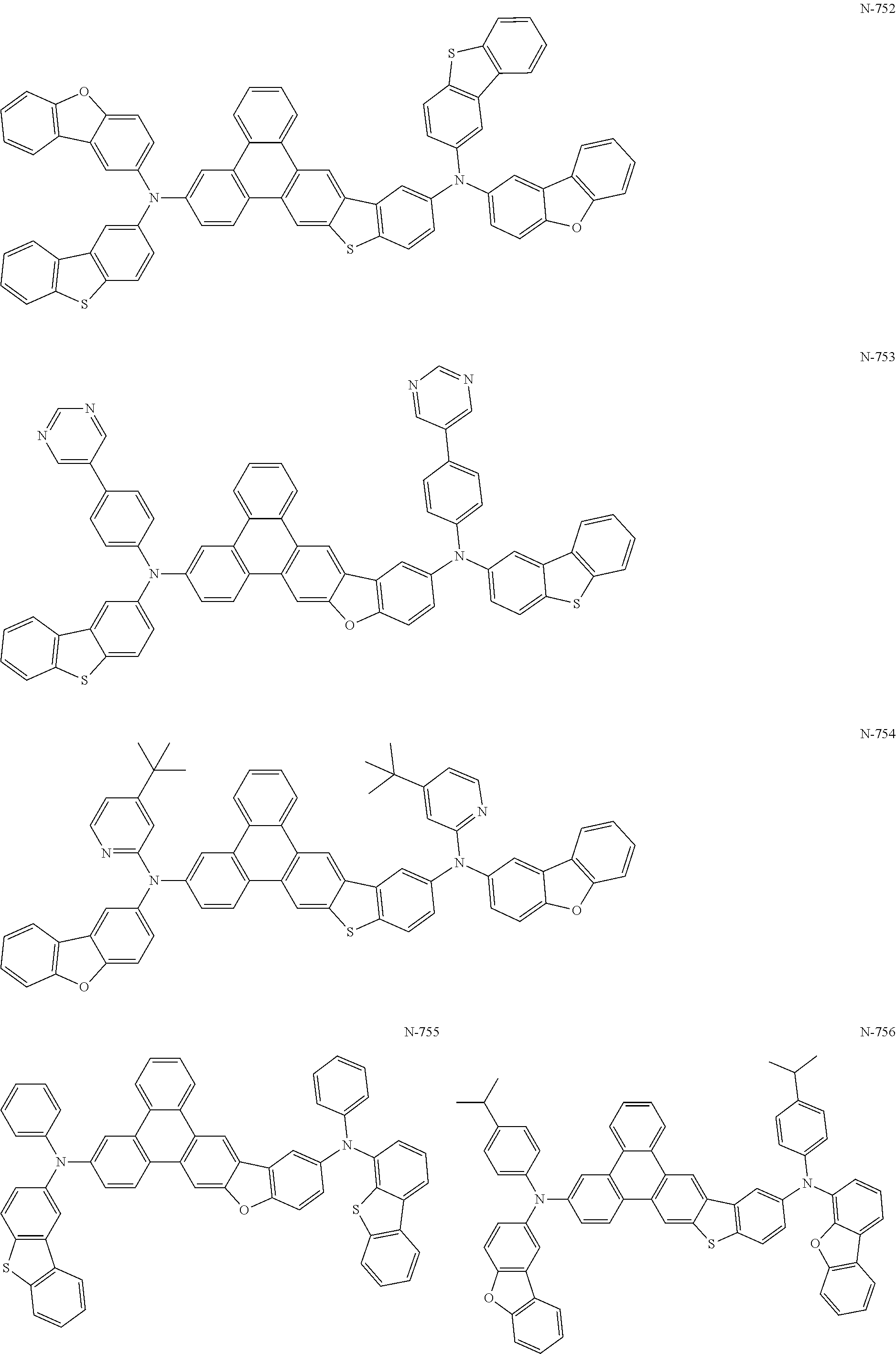

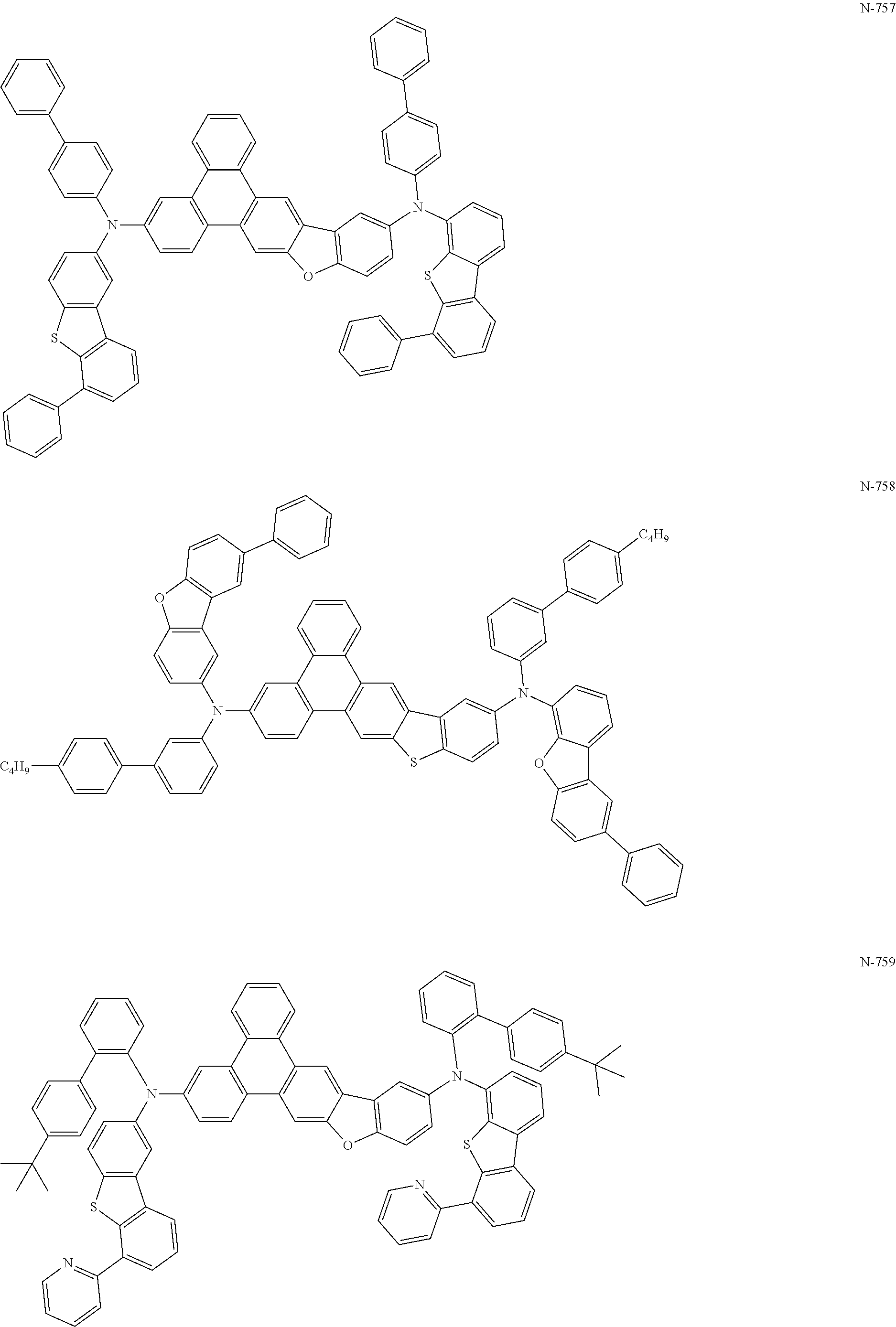

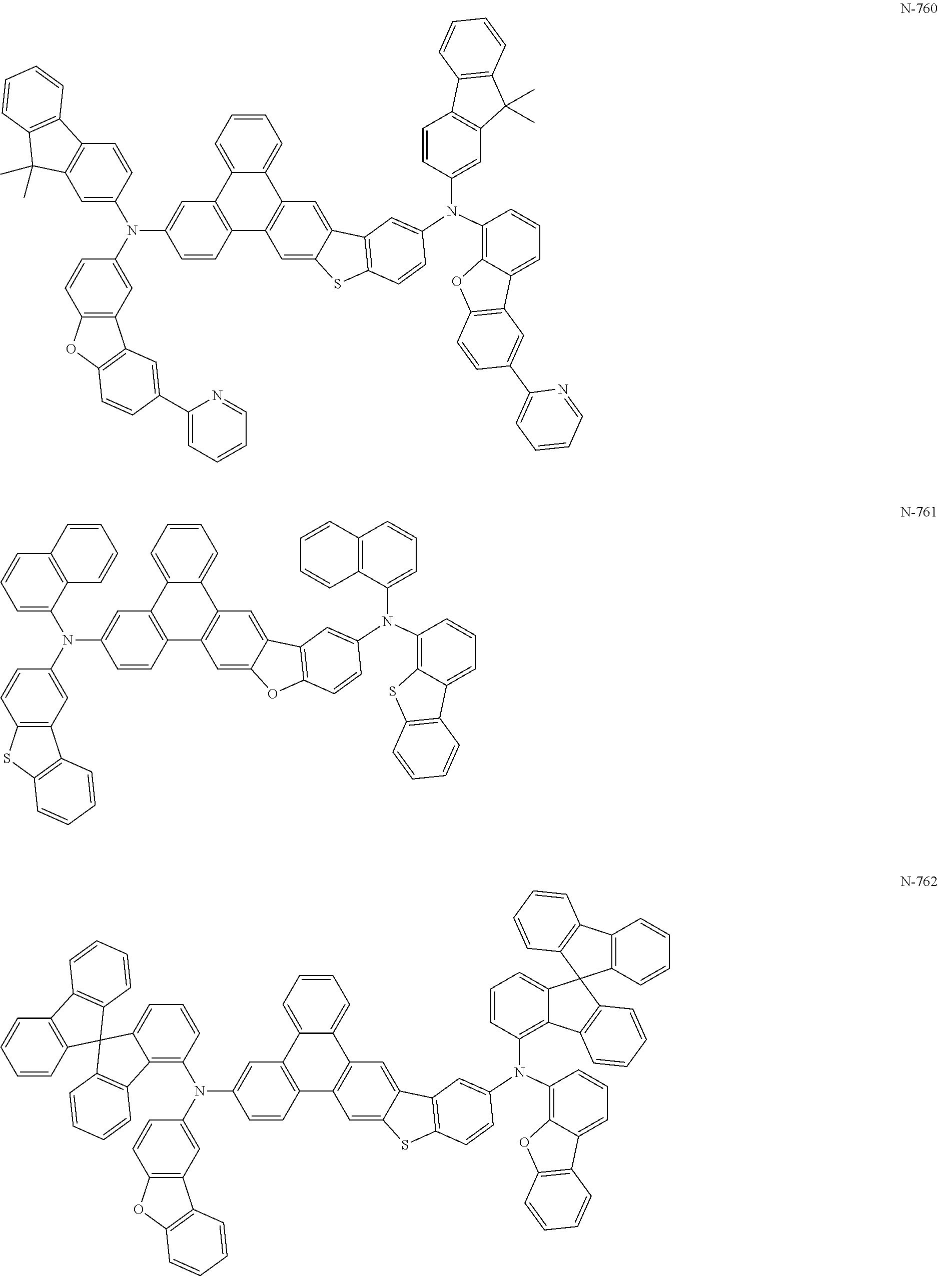 [SMILES: CC1(C)c2ccccc2-c2ccc(N(c3ccc4oc5ccc(-c6ccccn6)cc5c4c3)c3ccc4c(c3)c3ccccc3c3cc5c(cc43)sc3ccc(N(c4ccc6c(c4)C(C)(C)c4ccccc4-6)c4cccc6c4oc4ccc(-c7ccccn7)cc46)cc35)cc21 ; c1ccc2c(N(c3ccc4sc5ccccc5c4c3)c3ccc4c(c3)c3ccccc3c3cc5c(cc43)oc3ccc(N(c4cccc6ccccc46)c4cccc6c4sc4ccccc46)cc35)cccc2c1 ; c1ccc2c(c1)-c1ccccc1C21c2ccccc2-c2c(N(c3ccc4oc5ccccc5c4c3)c3ccc4c(c3)c3ccccc3c3cc5c(cc43)sc3ccc(N(c4cccc6c4-c4ccccc4C64c6ccccc6-c6ccccc64)c4cccc6c4oc4ccccc46)cc35)cccc21]

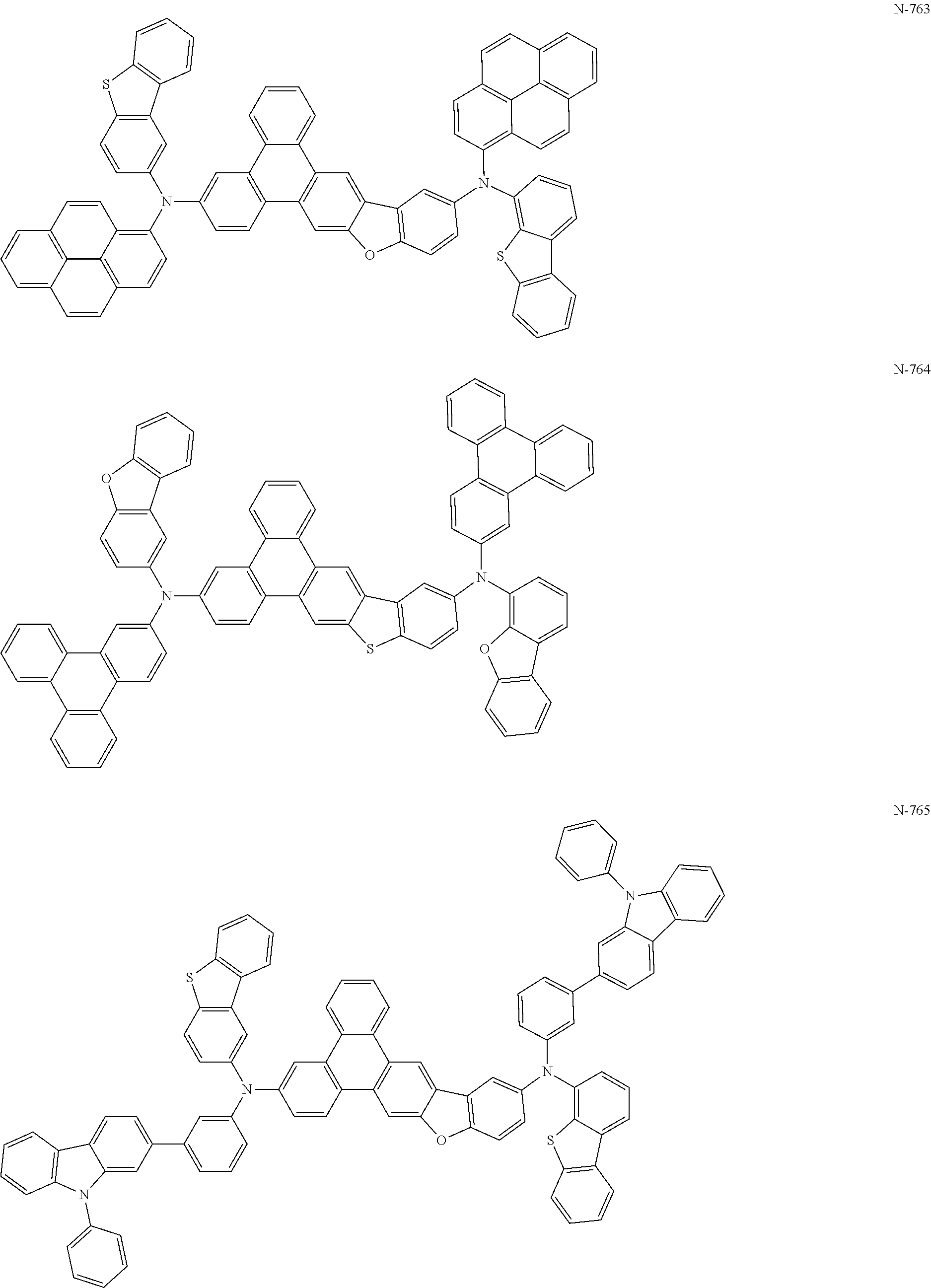 [SMILES: c1cc2ccc3ccc(N(c4ccc5sc6ccccc6c5c4)c4ccc5c(c4)c4ccccc4c4cc6c(cc54)oc4ccc(N(c5ccc7ccc8cccc9ccc5c7c89)c5cccc7c5sc5ccccc57)cc46)c4ccc(c1)c2c34 ; c1ccc2c(c1)oc1ccc(N(c3ccc4c5ccccc5c5ccccc5c4c3)c3ccc4c(c3)c3ccccc3c3cc5c(cc43)sc3ccc(N(c4ccc6c7ccccc7c7ccccc7c6c4)c4cccc6c4oc4ccccc46)cc35)cc12 ; c1ccc(-n2c3ccccc3c3ccc(-c4cccc(N(c5ccc6sc7ccccc7c6c5)c5ccc6c(c5)c5ccccc5c5cc7c(cc65)oc5ccc(N(c6cccc(-c8ccc9c%10ccccc%10n(-c%10ccccc%10)c9c8)c6)c6cccc8c6sc6ccccc68)cc57)c4)cc32)cc1]

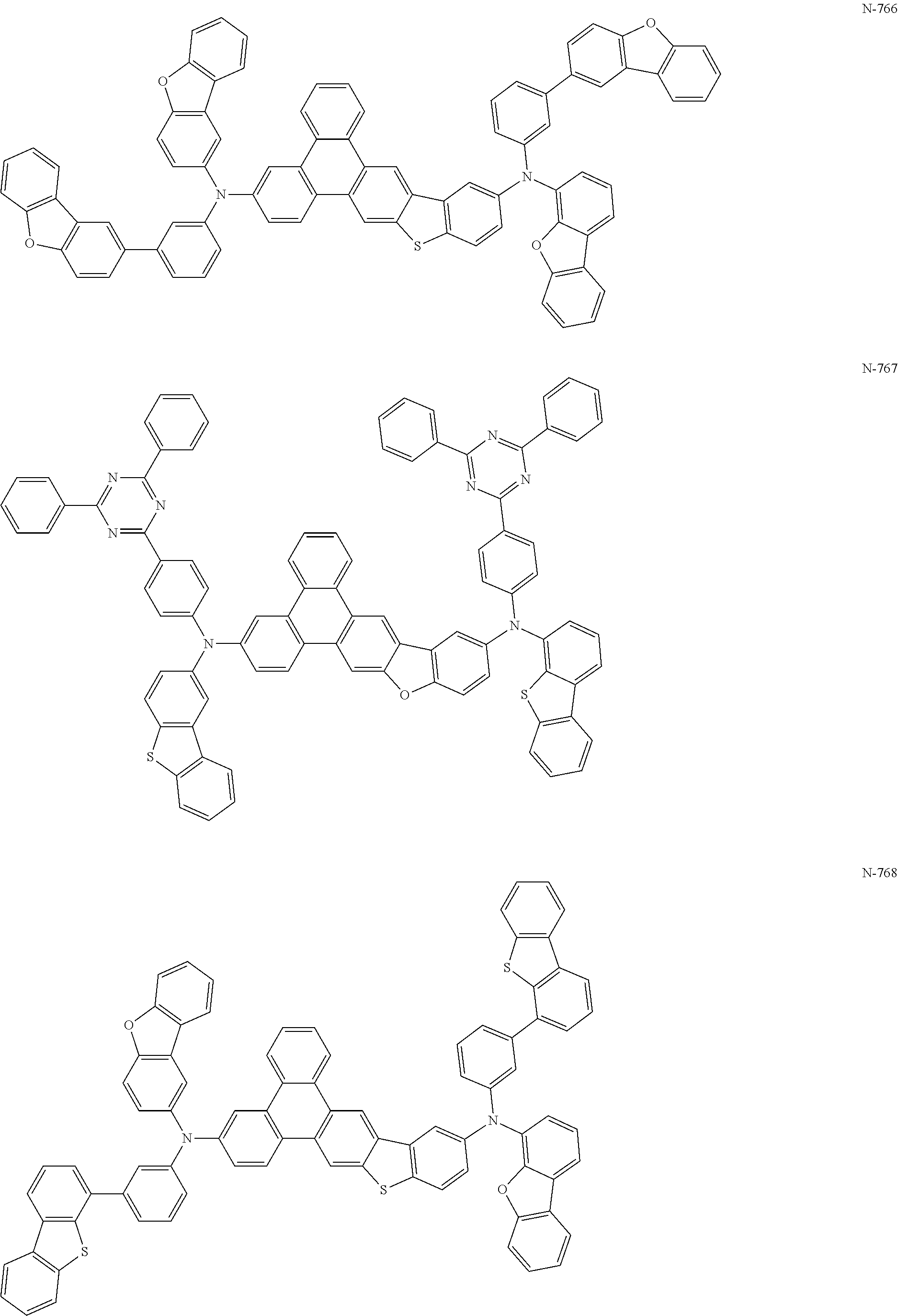 [SMILES: c1cc(-c2ccc3oc4ccccc4c3c2)cc(N(c2ccc3oc4ccccc4c3c2)c2ccc3c(c2)c2ccccc2c2cc4c(cc32)sc2ccc(N(c3cccc(-c5ccc6oc7ccccc7c6c5)c3)c3cccc5c3oc3ccccc35)cc24)c1 ; c1ccc(-c2nc(-c3ccccc3)nc(-c3ccc(N(c4ccc5sc6ccccc6c5c4)c4ccc5c(c4)c4ccccc4c4cc6c(cc54)oc4ccc(N(c5ccc(-c7nc(-c8ccccc8)nc(-c8ccccc8)n7)cc5)c5cccc7c5sc5ccccc57)cc46)cc3)n2)cc1 ; c1cc(-c2cccc3c2sc2ccccc23)cc(N(c2ccc3oc4ccccc4c3c2)c2ccc3c(c2)c2ccccc2c2cc4c(cc32)sc2ccc(N(c3cccc(-c5cccc6c5sc5ccccc56)c3)c3cccc5c3oc3ccccc35)cc24)c1]

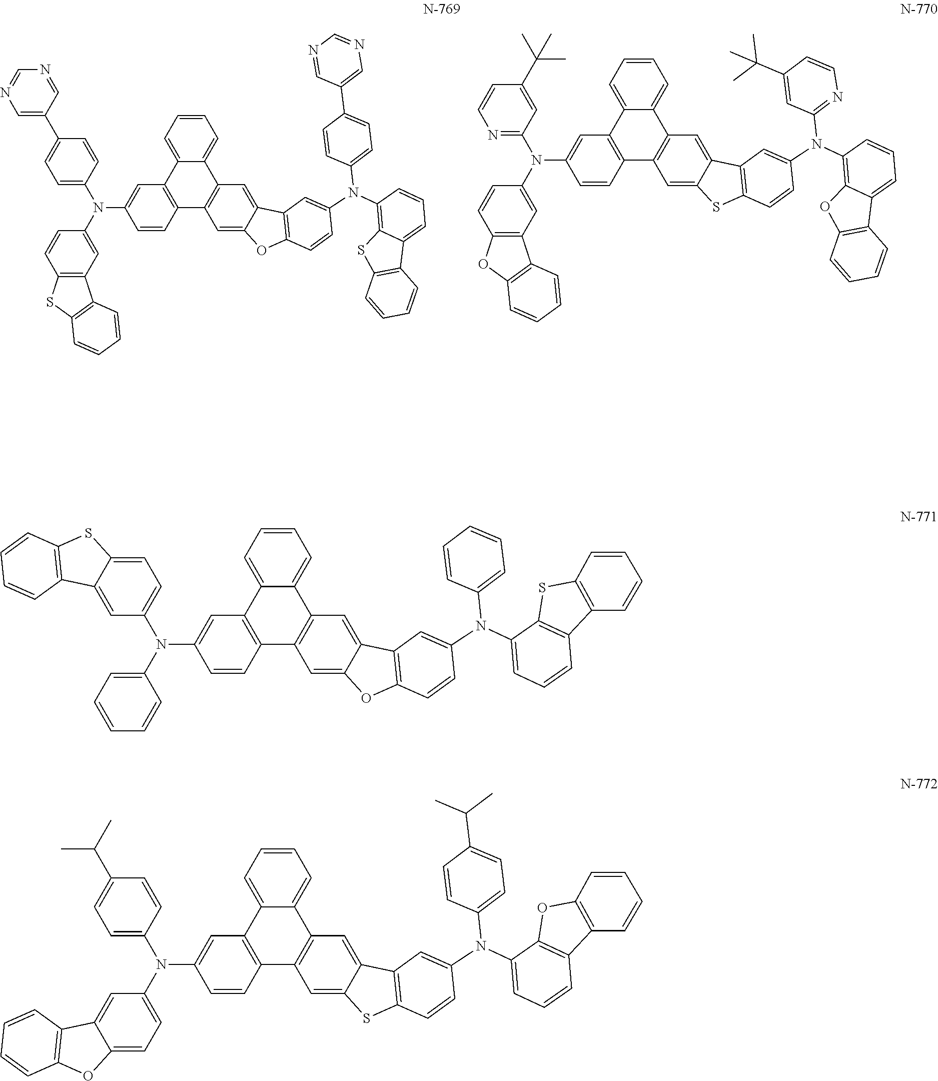 [SMILES: c1ccc2c(c1)sc1ccc(N(c3ccc(-c4cncnc4)cc3)c3ccc4c(c3)c3ccccc3c3cc5c(cc43)oc3ccc(N(c4ccc(-c6cncnc6)cc4)c4cccc6c4sc4ccccc46)cc35)cc12 ; CC(C)(C)c1ccnc(N(c2ccc3oc4ccccc4c3c2)c2ccc3c(c2)c2ccccc2c2cc4c(cc32)sc2ccc(N(c3cc(C(C)(C)C)ccn3)c3cccc5c3oc3ccccc35)cc24)c1 ; c1ccc(N(c2ccc3sc4ccccc4c3c2)c2ccc3c(c2)c2ccccc2c2cc4c(cc32)oc2ccc(N(c3ccccc3)c3cccc5c3sc3ccccc35)cc24)cc1 ; CC(C)c1ccc(N(c2ccc3oc4ccccc4c3c2)c2ccc3c(c2)c2ccccc2c2cc4c(cc32)sc2ccc(N(c3ccc(C(C)C)cc3)c3cccc5c3oc3ccccc35)cc24)cc1]

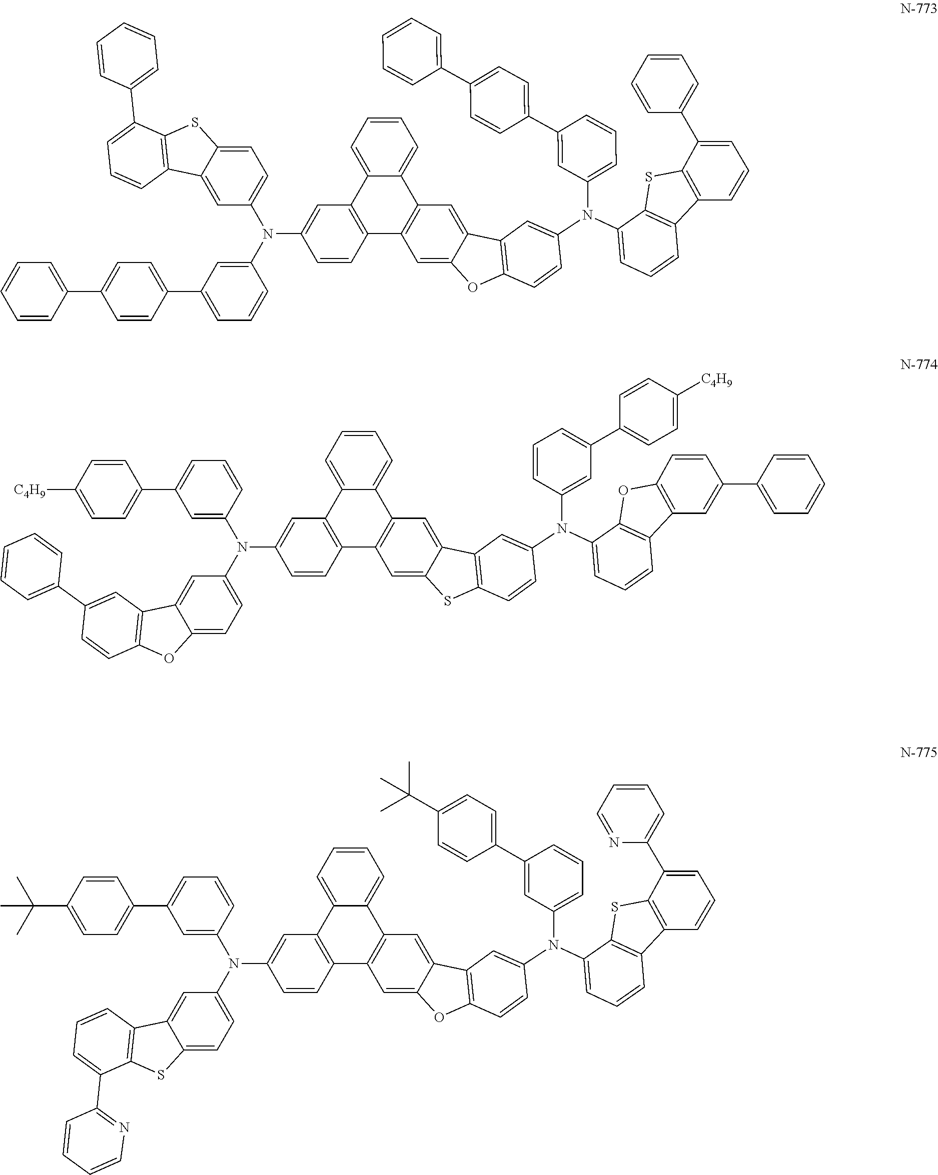

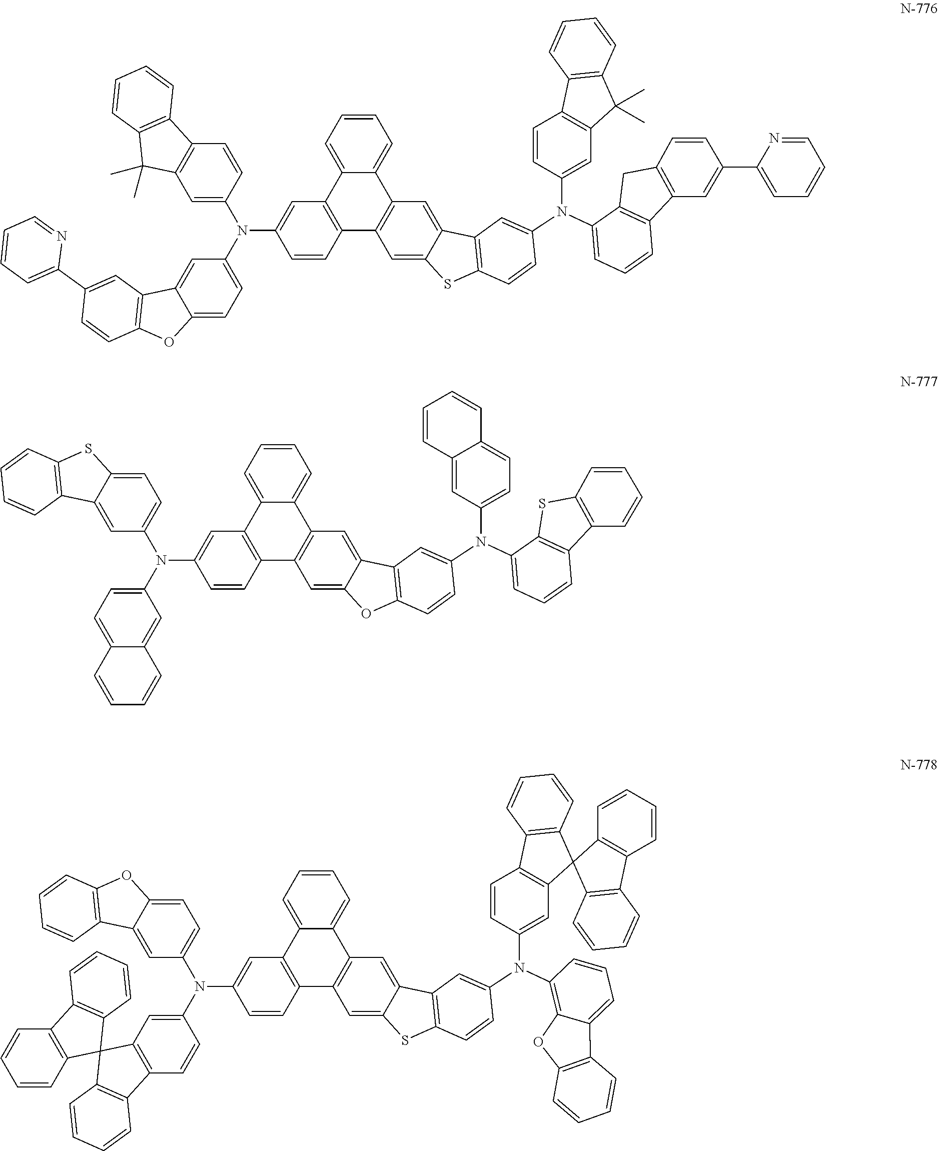 [SMILES: CC1(C)c2ccccc2-c2ccc(N(c3ccc4oc5ccc(-c6ccccn6)cc5c4c3)c3ccc4c(c3)c3ccccc3c3cc5c(cc43)sc3ccc(N(c4ccc6c(c4)C(C)(C)c4ccccc4-6)c4cccc6c4Cc4ccc(-c7ccccn7)cc4-6)cc35)cc21 ; c1ccc2cc(N(c3ccc4sc5ccccc5c4c3)c3ccc4c(c3)c3ccccc3c3cc5c(cc43)oc3ccc(N(c4ccc6ccccc6c4)c4cccc6c4sc4ccccc46)cc35)ccc2c1 ; c1ccc2c(c1)-c1ccccc1C21c2ccccc2-c2ccc(N(c3ccc4oc5ccccc5c4c3)c3ccc4c(c3)c3ccccc3c3cc5c(cc43)sc3ccc(N(c4ccc6c(c4)C4(c7ccccc7-c7ccccc74)c4ccccc4-6)c4cccc6c4oc4ccccc46)cc35)cc21]

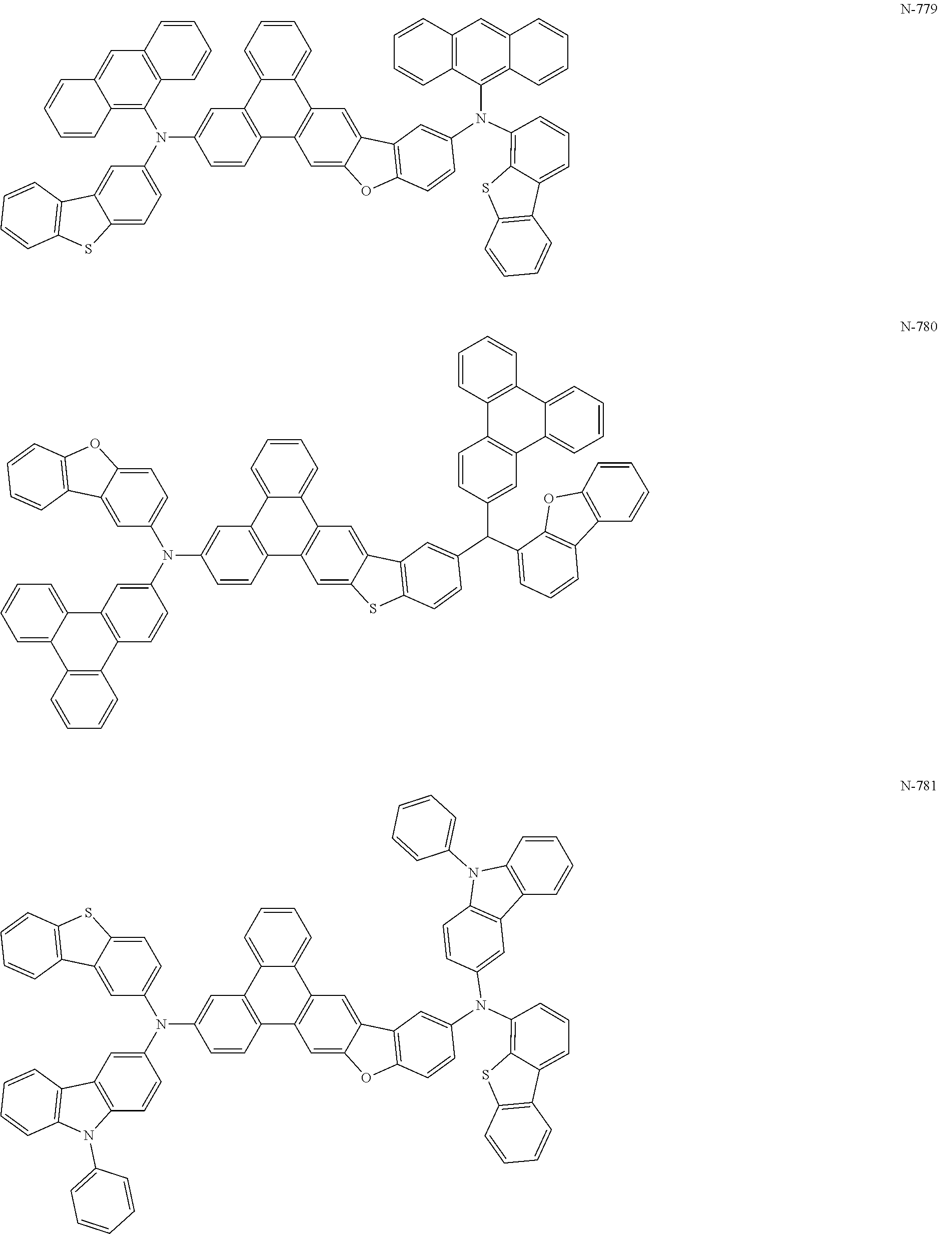 [SMILES: c1ccc2c(N(c3ccc4sc5ccccc5c4c3)c3ccc4c(c3)c3ccccc3c3cc5c(cc43)oc3ccc(N(c4c6ccccc6cc6ccccc46)c4cccc6c4sc4ccccc46)cc35)c3ccccc3cc2c1 ; c1ccc2c(c1)oc1ccc(N(c3ccc4c5ccccc5c5ccccc5c4c3)c3ccc4c(c3)c3ccccc3c3cc5c(cc43)sc3ccc(C(c4ccc6c7ccccc7c7ccccc7c6c4)c4cccc6c4oc4ccccc46)cc35)cc12 ; c1ccc(-n2c3ccccc3c3cc(N(c4ccc5sc6ccccc6c5c4)c4ccc5c(c4)c4ccccc4c4cc6c(cc54)oc4ccc(N(c5ccc7c(c5)c5ccccc5n7-c5ccccc5)c5cccc7c5sc5ccccc57)cc46)ccc32)cc1]

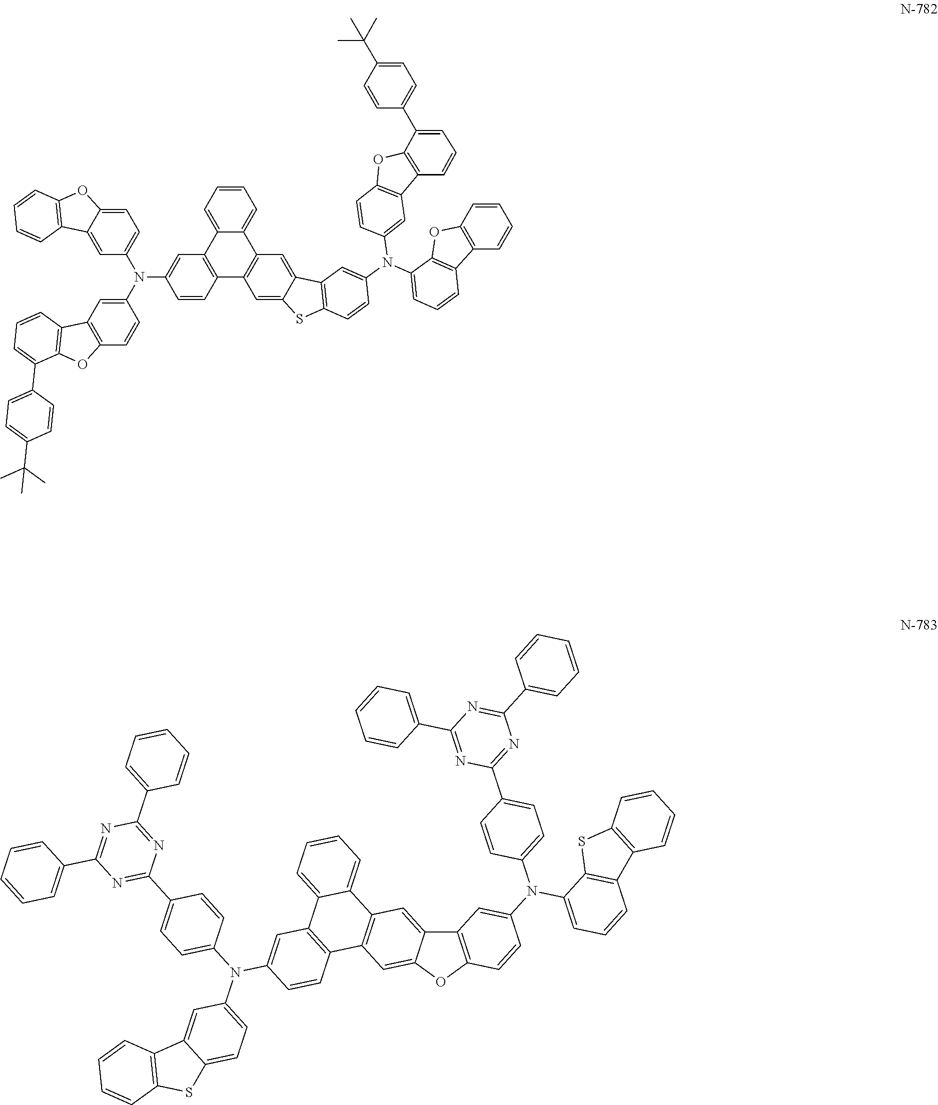 [SMILES: CC(C)(C)c1ccc(-c2cccc3c2oc2ccc(N(c4ccc5oc6ccccc6c5c4)c4ccc5c(c4)c4ccccc4c4cc6c(cc54)sc4ccc(N(c5ccc7oc8c(-c9ccc(C(C)(C)C)cc9)cccc8c7c5)c5cccc7c5oc5ccccc57)cc46)cc23)cc1 ; c1ccc(-c2nc(-c3ccccc3)nc(-c3ccc(N(c4ccc5sc6ccccc6c5c4)c4ccc5c(c4)c4ccccc4c4cc6c(cc54)oc4ccc(N(c5ccc(-c7nc(-c8ccccc8)nc(-c8ccccc8)n7)cc5)c5cccc7c5sc5ccccc57)cc46)cc3)n2)cc1]

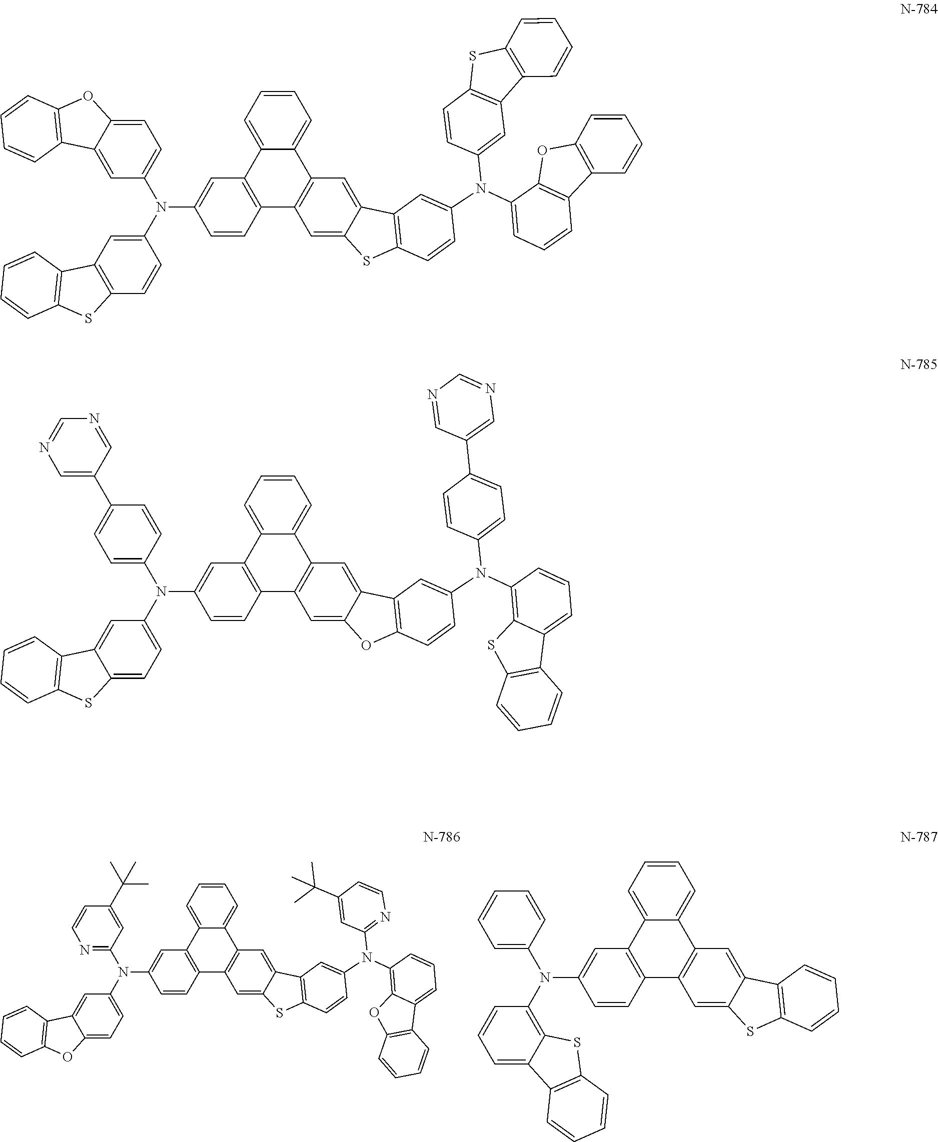 [SMILES: c1ccc2c(c1)oc1ccc(N(c3ccc4sc5ccccc5c4c3)c3ccc4c(c3)c3ccccc3c3cc5c(cc43)sc3ccc(N(c4ccc6sc7ccccc7c6c4)c4cccc6c4oc4ccccc46)cc35)cc12 ; c1ccc2c(c1)sc1ccc(N(c3ccc(-c4cncnc4)cc3)c3ccc4c(c3)c3ccccc3c3cc5c(cc43)oc3ccc(N(c4ccc(-c6cncnc6)cc4)c4cccc6c4sc4ccccc46)cc35)cc12 ; CC(C)(C)c1ccnc(N(c2ccc3oc4ccccc4c3c2)c2ccc3c(c2)c2ccccc2c2cc4c(cc32)sc2ccc(N(c3cc(C(C)(C)C)ccn3)c3cccc5c3oc3ccccc35)cc24)c1 ; c1ccc(N(c2ccc3c(c2)c2ccccc2c2cc4c(cc32)sc2ccccc24)c2cccc3c2sc2ccccc23)cc1]

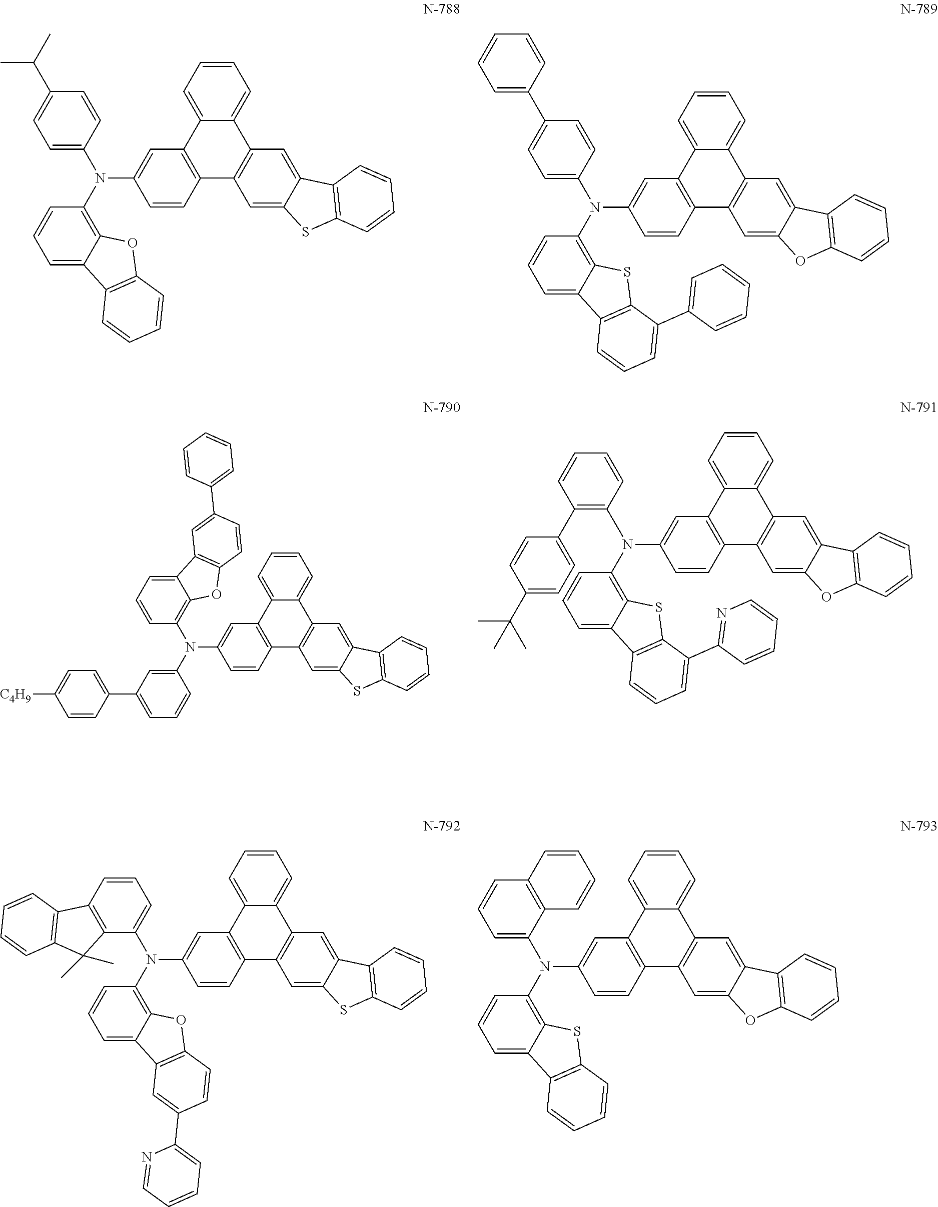

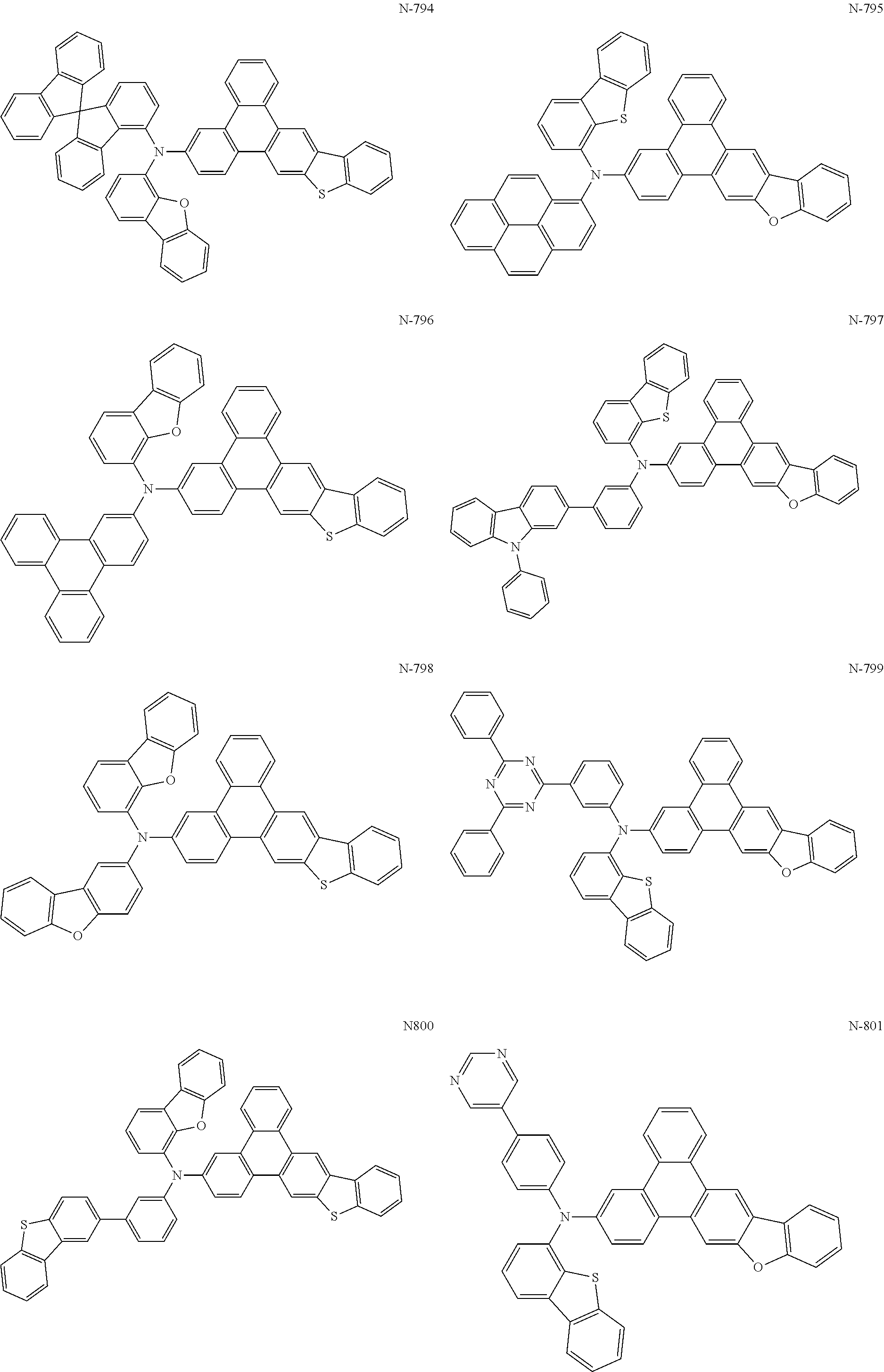

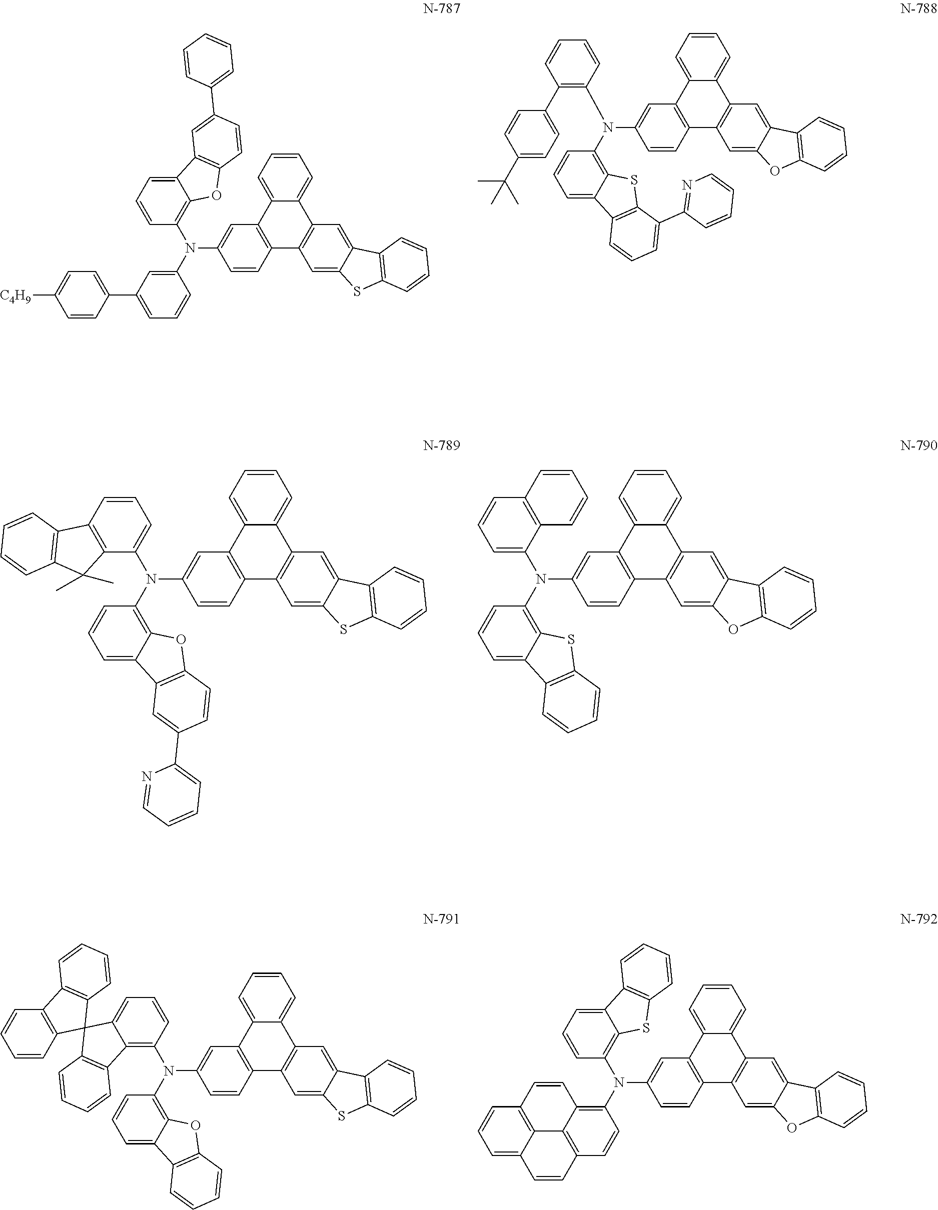

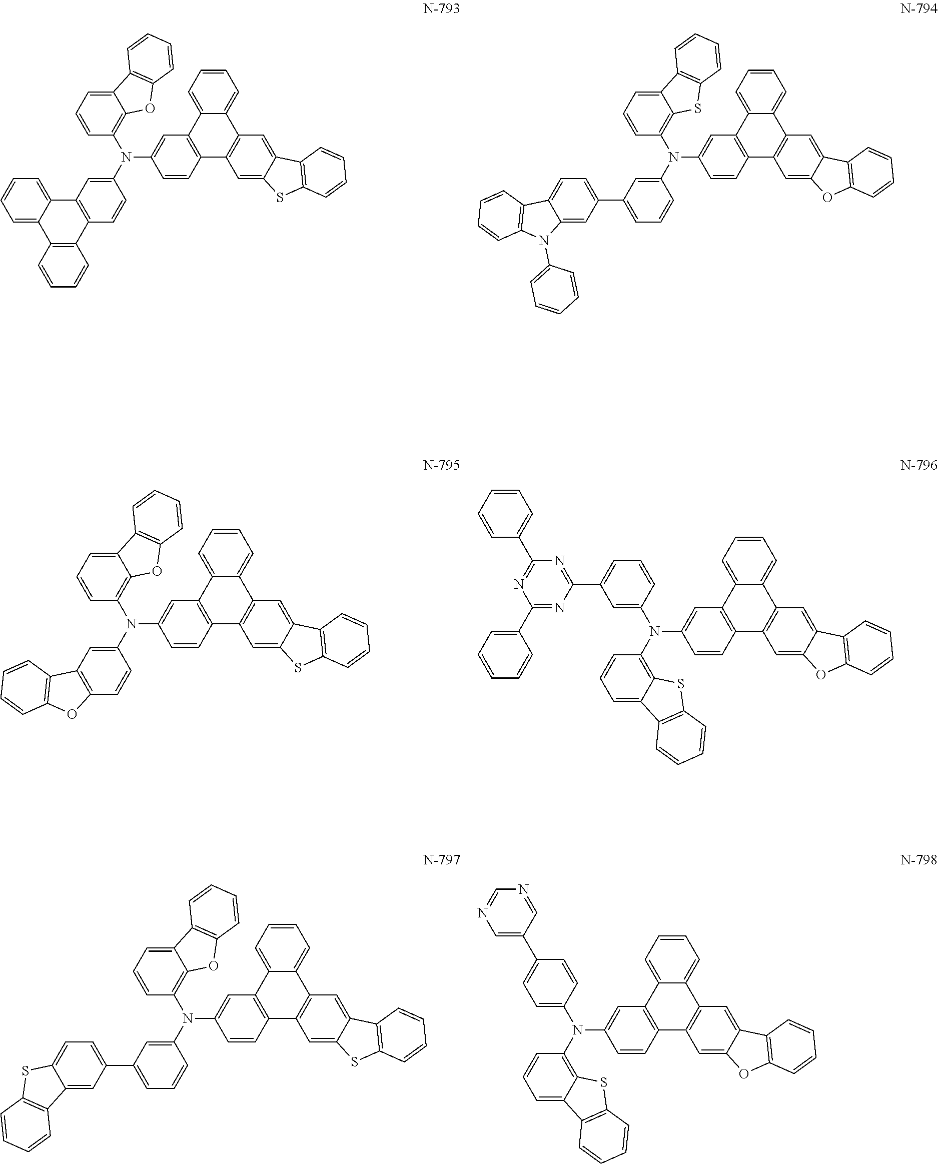

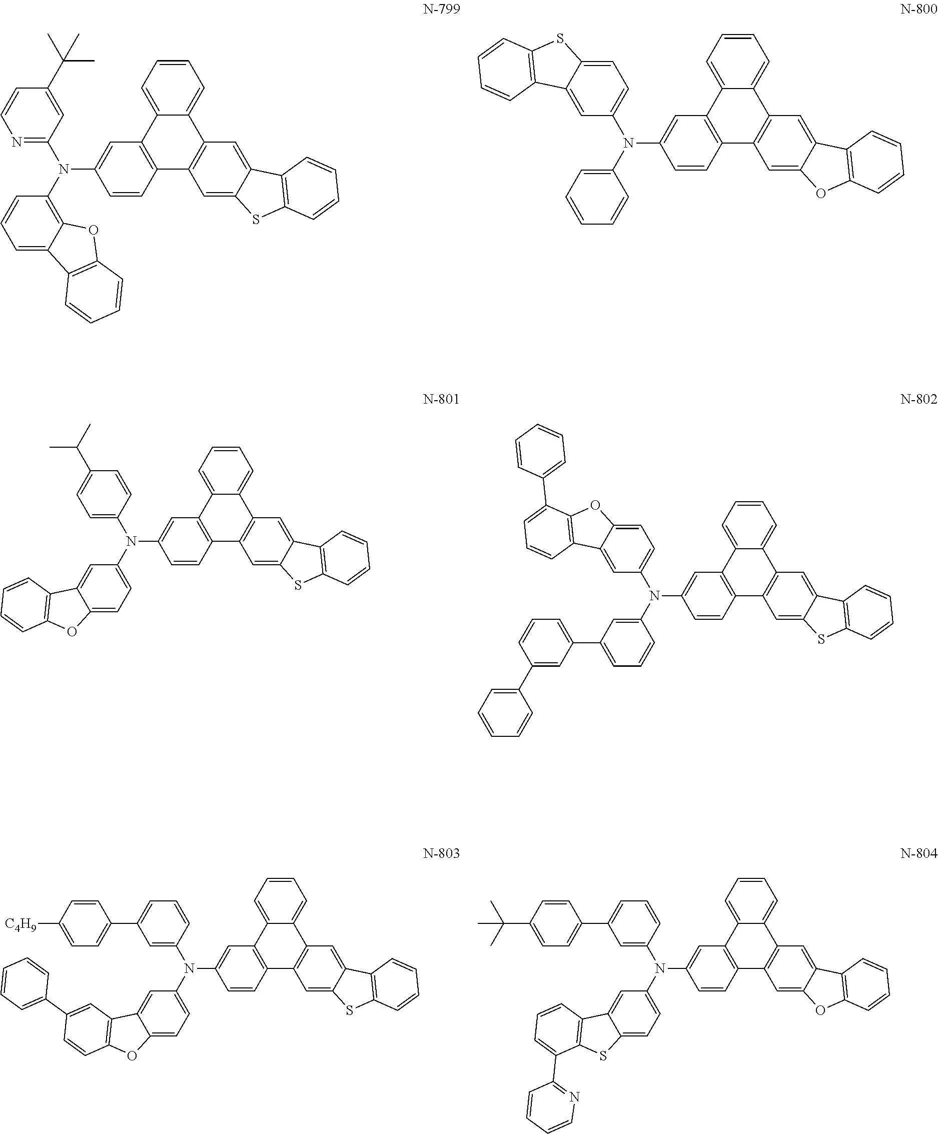

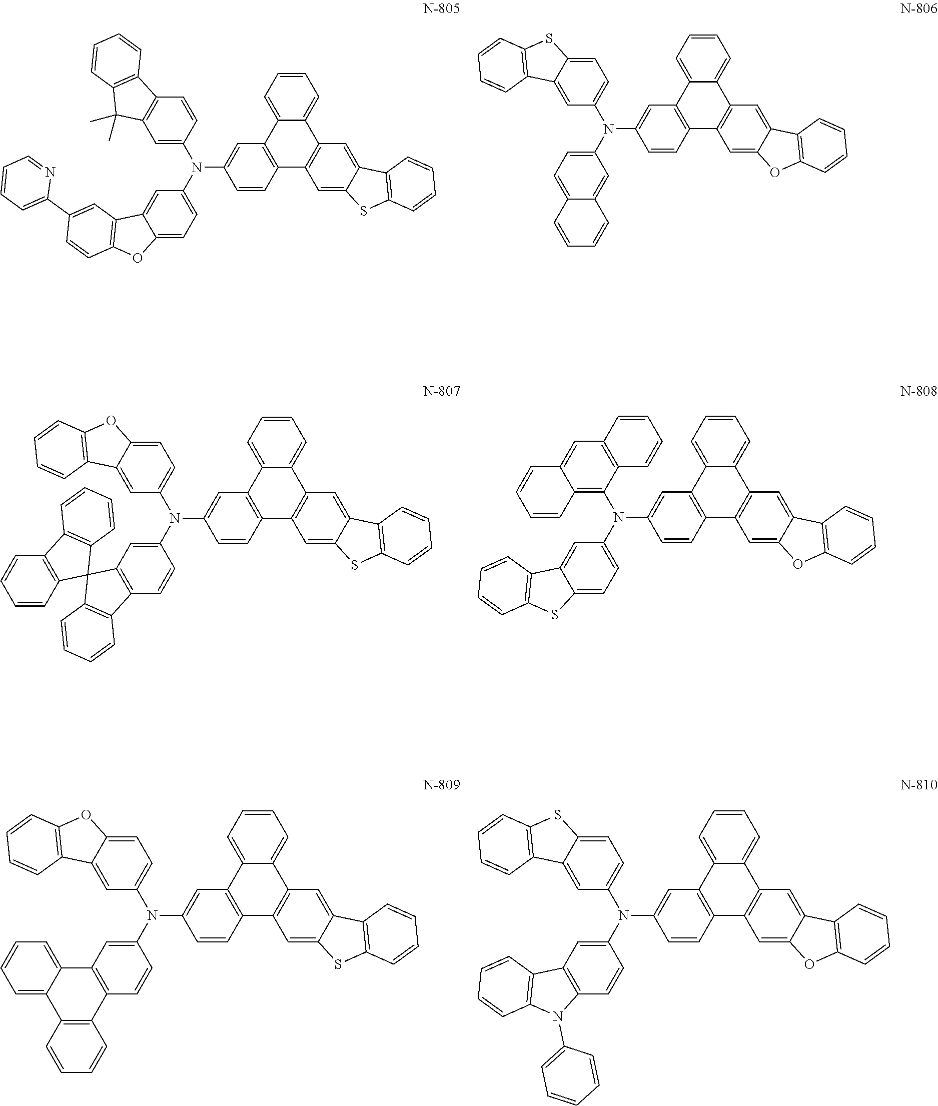

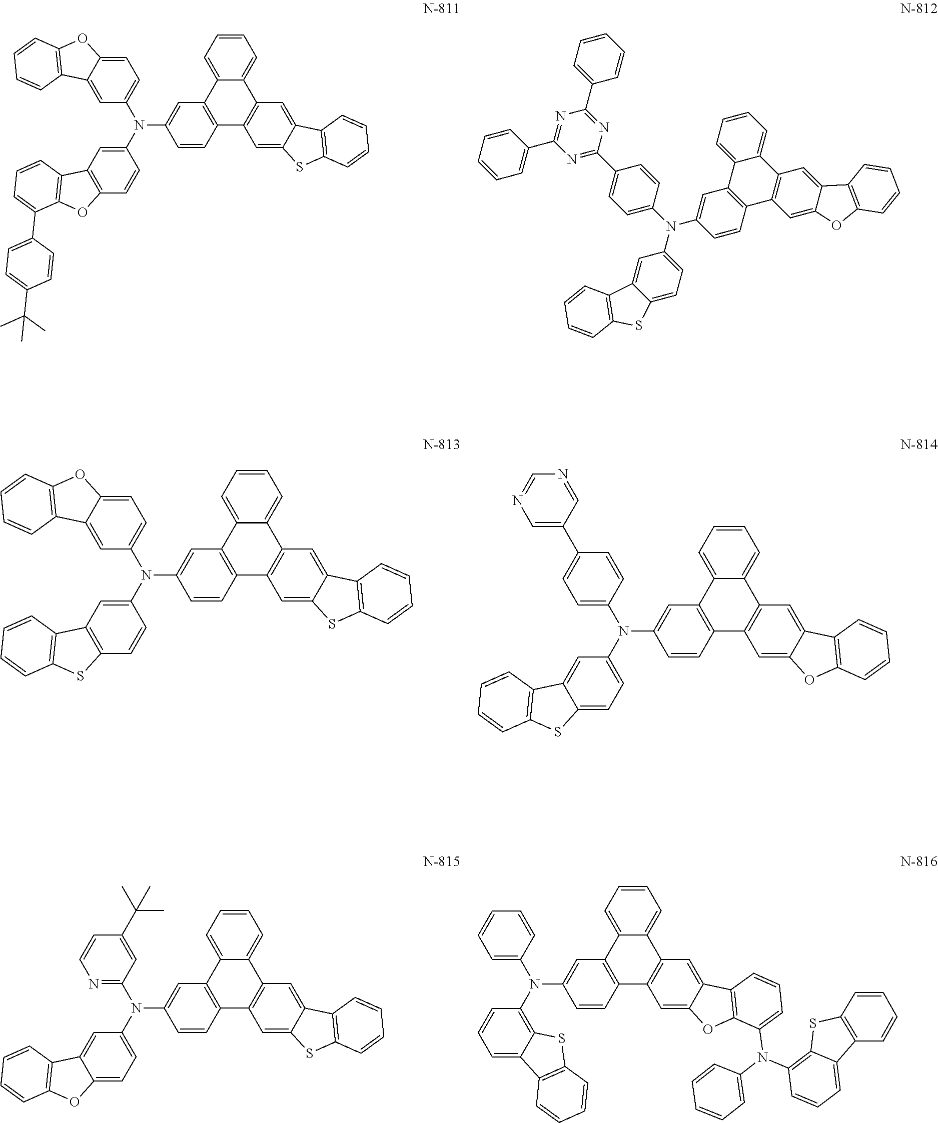

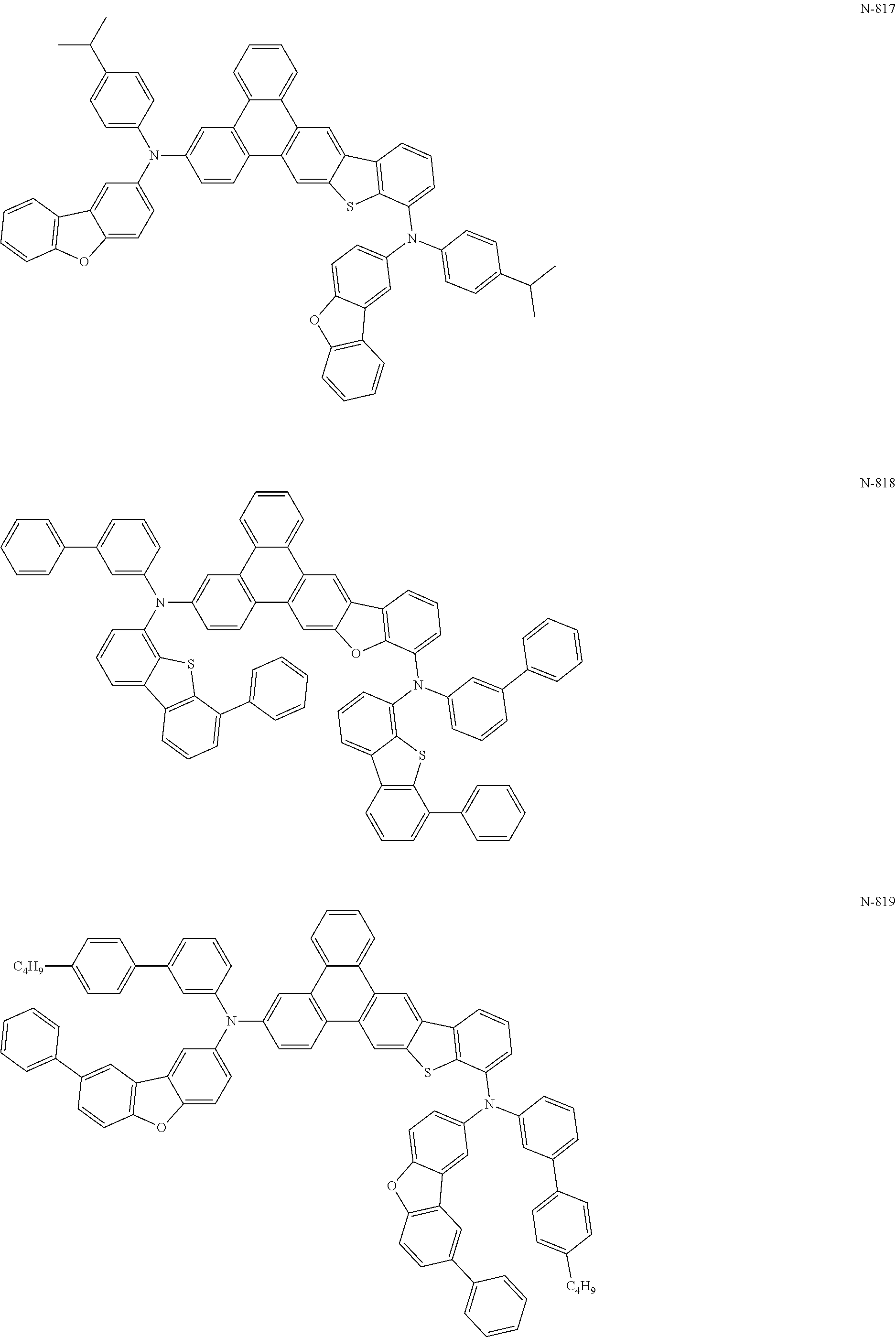

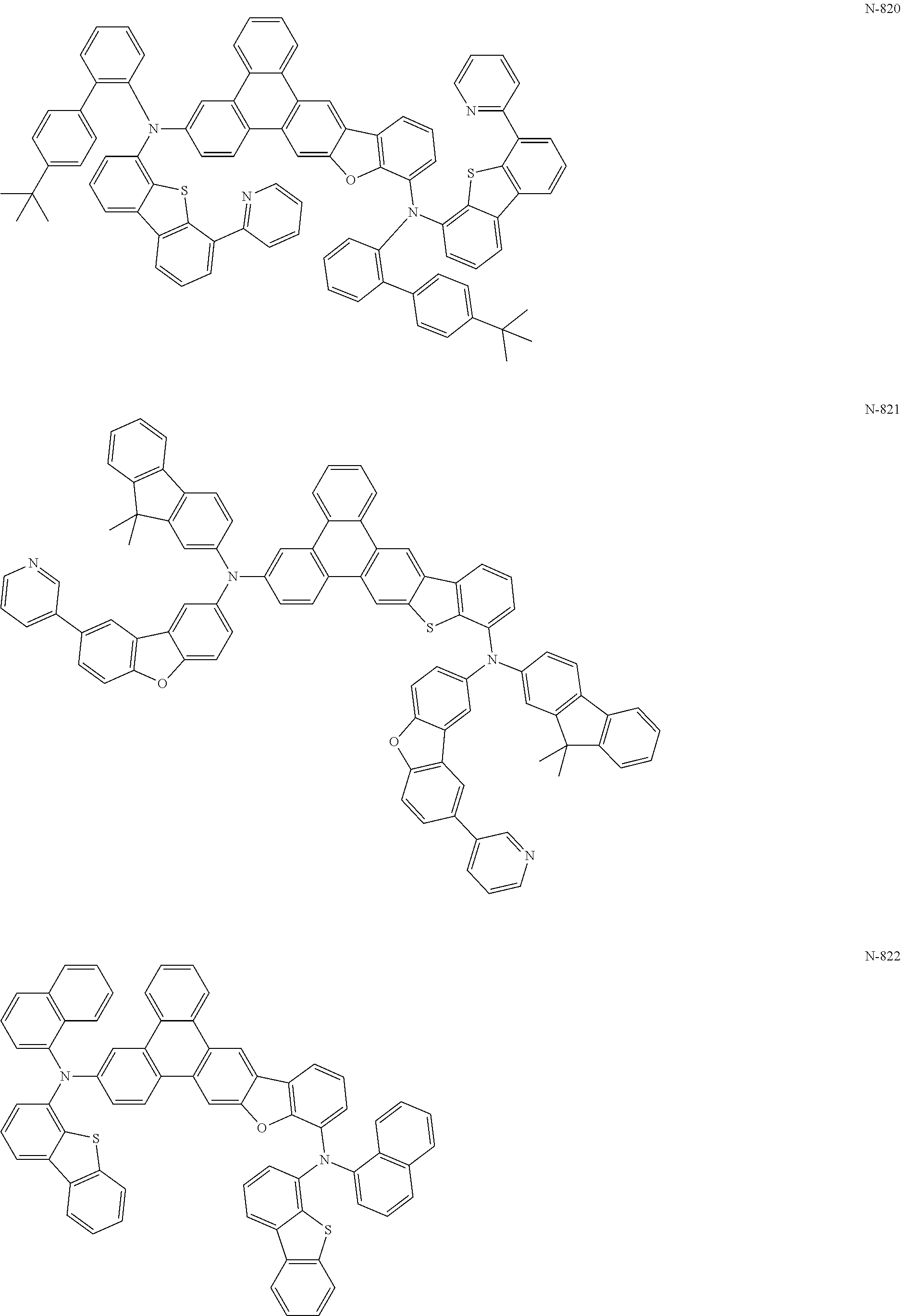 [SMILES: CC(C)(C)c1ccc(-c2ccccc2N(c2ccc3c(c2)c2ccccc2c2cc4c(cc32)oc2c(N(c3ccccc3-c3ccc(C(C)(C)C)cc3)c3cccc5c3sc3c(-c6ccccn6)cccc35)cccc24)c2cccc3c2sc2c(-c4ccccn4)cccc23)cc1 ; CC1(C)c2ccccc2-c2ccc(N(c3ccc4oc5ccc(-c6cccnc6)cc5c4c3)c3ccc4c(c3)c3ccccc3c3cc5c(cc43)sc3c(N(c4ccc6c(c4)C(C)(C)c4ccccc4-6)c4ccc6oc7ccc(-c8cccnc8)cc7c6c4)cccc35)cc21 ; c1ccc2c(N(c3ccc4c(c3)c3ccccc3c3cc5c(cc43)oc3c(N(c4cccc6ccccc46)c4cccc6c4sc4ccccc46)cccc35)c3cccc4c3sc3ccccc34)cccc2c1]

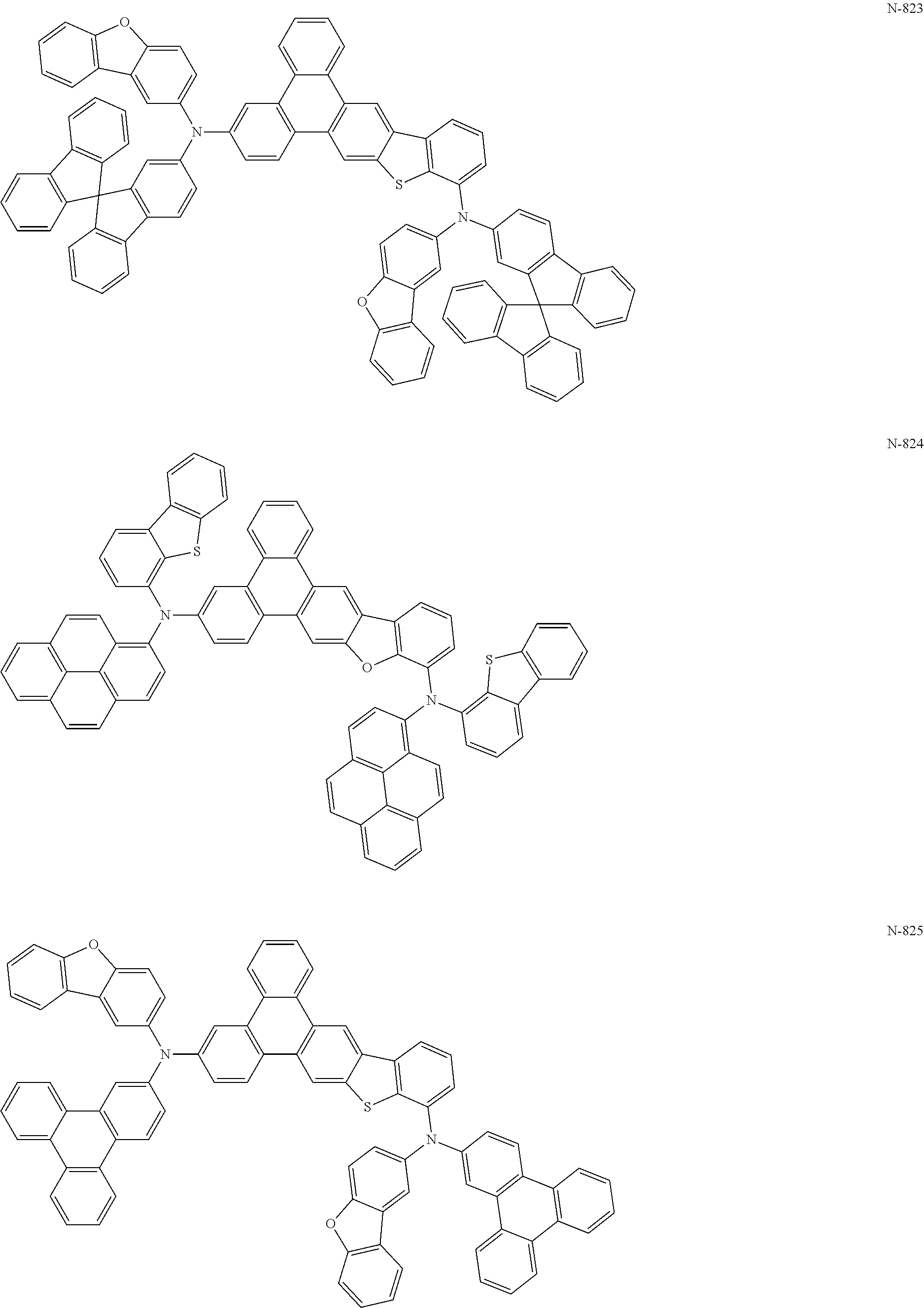

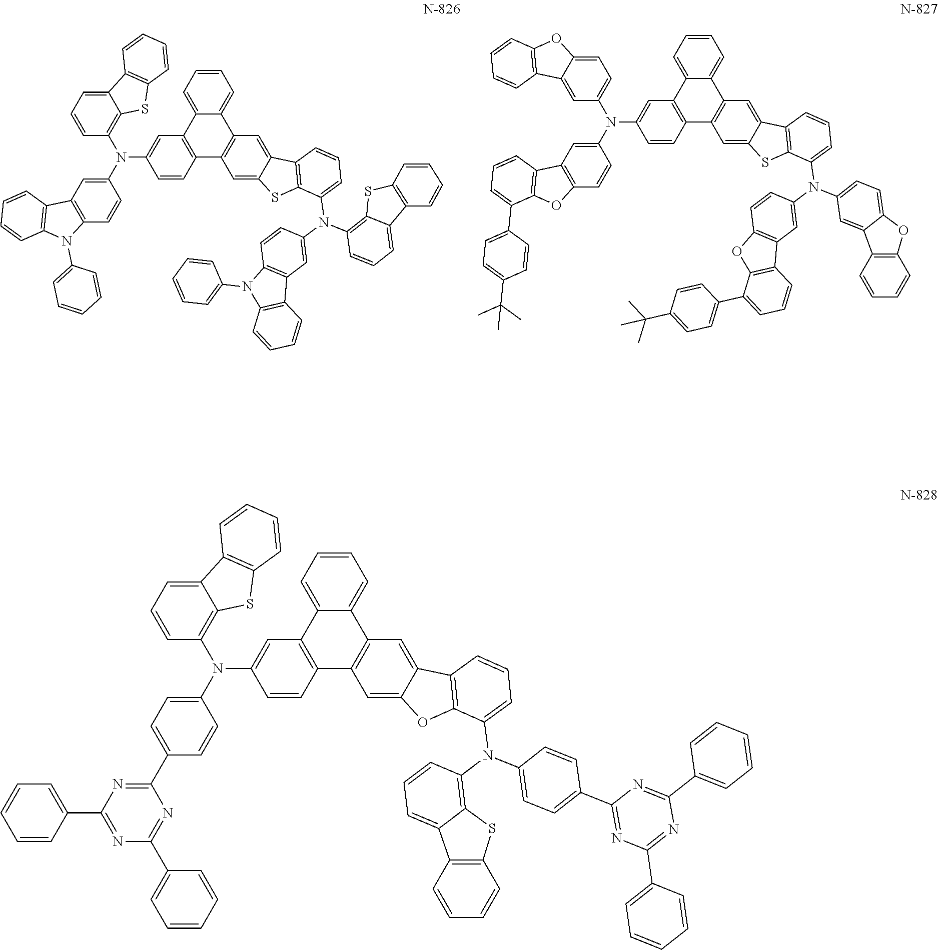 [SMILES: c1ccc(-n2c3ccccc3c3cc(N(c4ccc5c(c4)c4ccccc4c4cc6c(cc54)sc4c(N(c5ccc7c(c5)c5ccccc5n7-c5ccccc5)c5cccc7c5sc5ccccc57)cccc46)c4cccc5c4sc4ccccc45)ccc32)cc1 ; CC(C)(C)c1ccc(-c2cccc3c2oc2ccc(N(c4ccc5oc6ccccc6c5c4)c4ccc5c(c4)c4ccccc4c4cc6c(cc54)sc4c(N(c5ccc7oc8ccccc8c7c5)c5ccc7oc8c(-c9ccc(C(C)(C)C)cc9)cccc8c7c5)cccc46)cc23)cc1 ; c1ccc(-c2nc(-c3ccccc3)nc(-c3ccc(N(c4ccc5c(c4)c4ccccc4c4cc6c(cc54)oc4c(N(c5ccc(-c7nc(-c8ccccc8)nc(-c8ccccc8)n7)cc5)c5cccc7c5sc5ccccc57)cccc46)c4cccc5c4sc4ccccc45)cc3)n2)cc1]

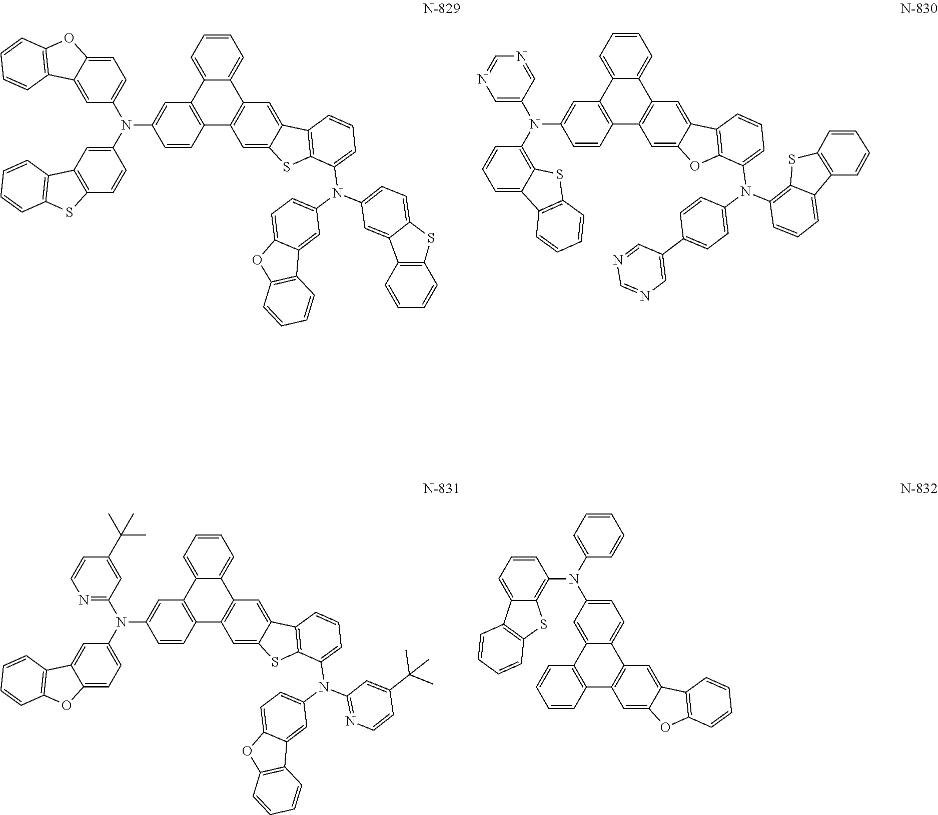 [SMILES: c1ccc2c(c1)oc1ccc(N(c3ccc4sc5ccccc5c4c3)c3ccc4c(c3)c3ccccc3c3cc5c(cc43)sc3c(N(c4ccc6oc7ccccc7c6c4)c4ccc6sc7ccccc7c6c4)cccc35)cc12 ; c1ccc2c(c1)sc1c(N(c3cncnc3)c3ccc4c(c3)c3ccccc3c3cc5c(cc43)oc3c(N(c4ccc(-c6cncnc6)cc4)c4cccc6c4sc4ccccc46)cccc35)cccc12 ; CC(C)(C)c1ccnc(N(c2ccc3oc4ccccc4c3c2)c2ccc3c(c2)c2ccccc2c2cc4c(cc32)sc2c(N(c3ccc5oc6ccccc6c5c3)c3cc(C(C)(C)C)ccn3)cccc24)c1 ; c1ccc(N(c2ccc3c(c2)c2ccccc2c2cc4oc5ccccc5c4cc32)c2cccc3c2sc2ccccc23)cc1]

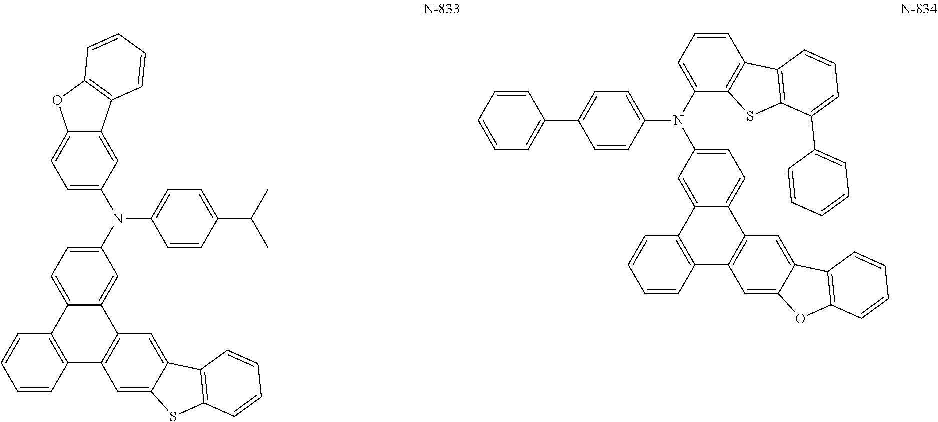 [SMILES: CC(C)c1ccc(N(c2ccc3oc4ccccc4c3c2)c2ccc3c4ccccc4c4cc5sc6ccccc6c5cc4c3c2)cc1 ; c1ccc(-c2ccc(N(c3ccc4c(c3)c3ccccc3c3cc5oc6ccccc6c5cc43)c3cccc4c3sc3c(-c5ccccc5)cccc34)cc2)cc1]

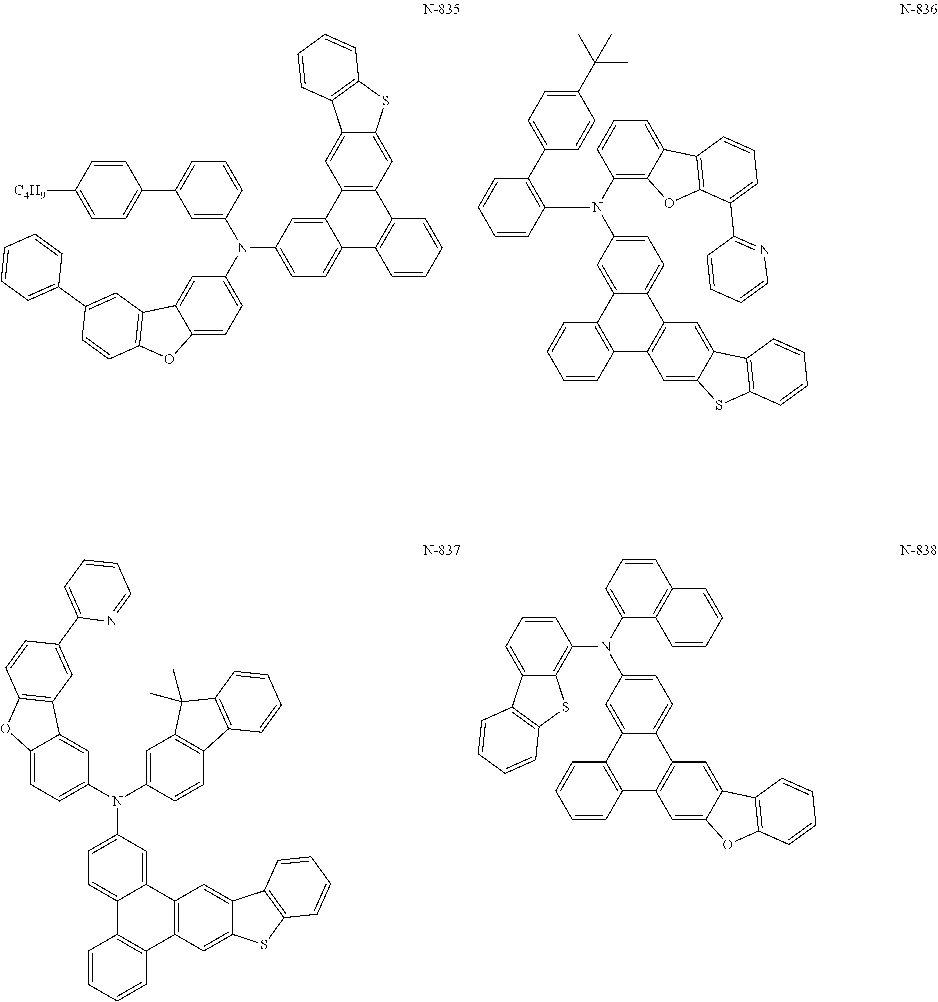

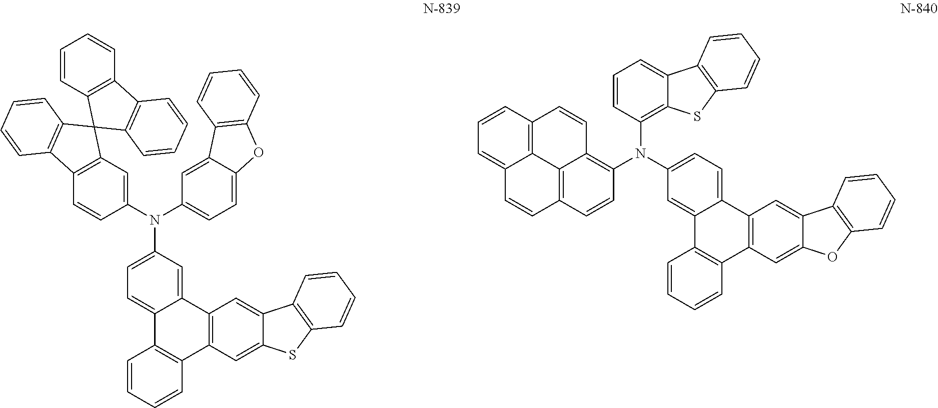 [SMILES: c1ccc2c(c1)-c1ccccc1C21c2ccccc2-c2ccc(N(c3ccc4oc5ccccc5c4c3)c3ccc4c5ccccc5c5cc6sc7ccccc7c6cc5c4c3)cc21 ; c1cc2ccc3ccc(N(c4ccc5c(c4)c4ccccc4c4cc6oc7ccccc7c6cc54)c4cccc5c4sc4ccccc45)c4ccc(c1)c2c34]

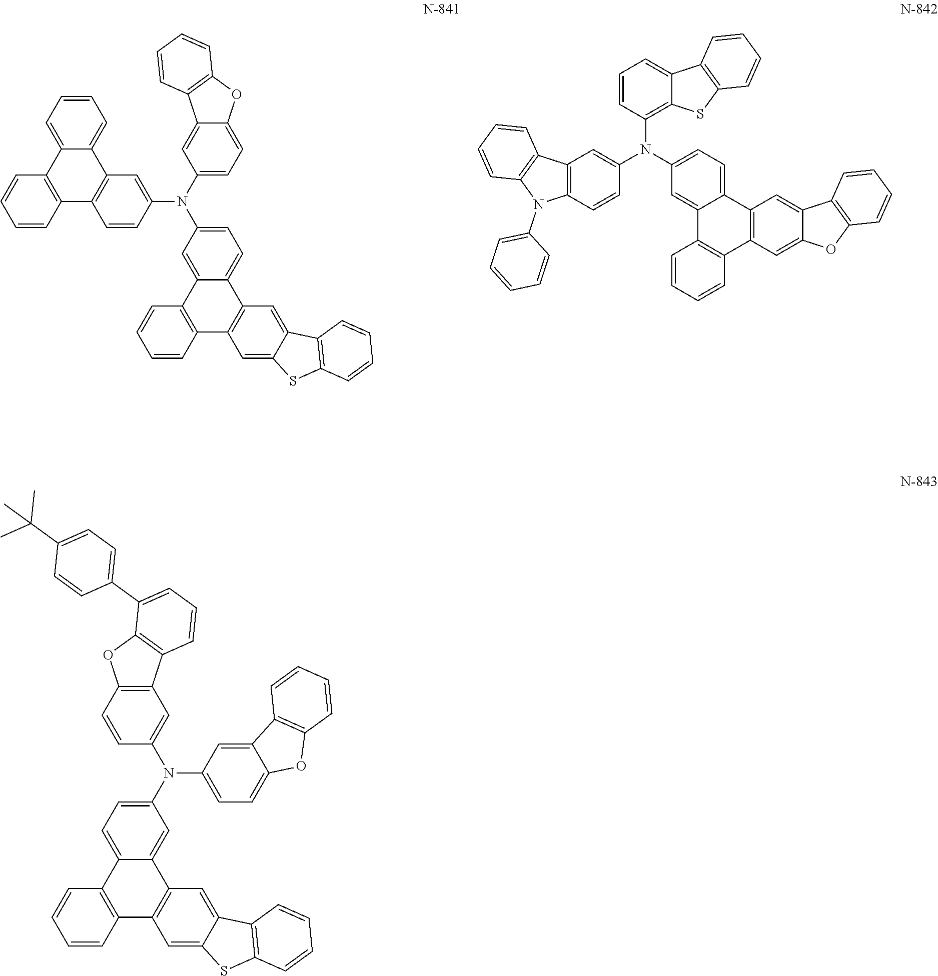 [SMILES: c1ccc2c(c1)oc1ccc(N(c3ccc4c5ccccc5c5ccccc5c4c3)c3ccc4c(c3)c3ccccc3c3cc5sc6ccccc6c5cc43)cc12 ; c1ccc(-n2c3ccccc3c3cc(N(c4ccc5c(c4)c4ccccc4c4cc6oc7ccccc7c6cc54)c4cccc5c4sc4ccccc45)ccc32)cc1 ; CC(C)(C)c1ccc(-c2cccc3c2oc2ccc(N(c4ccc5oc6ccccc6c5c4)c4ccc5c6ccccc6c6cc7sc8ccccc8c7cc6c5c4)cc23)cc1]

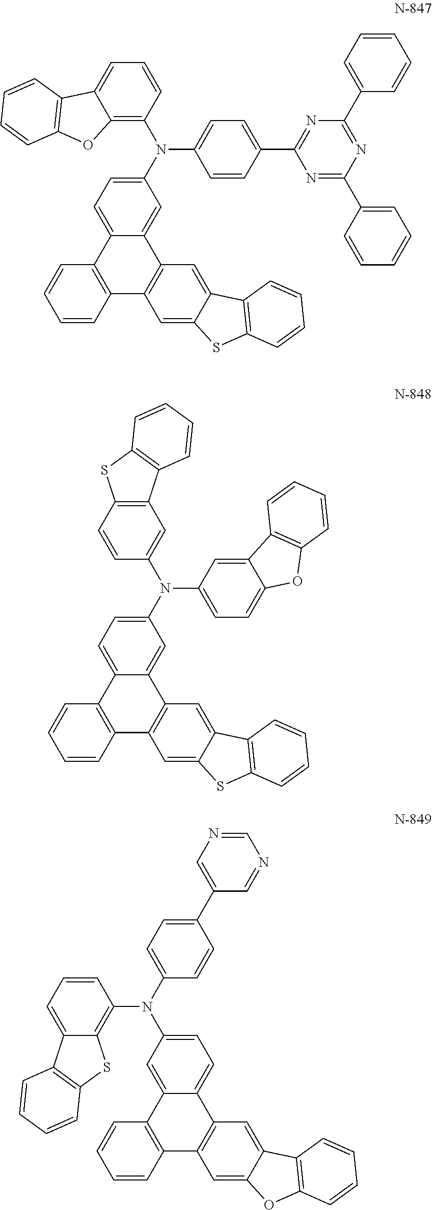 [SMILES: c1ccc(-c2nc(-c3ccccc3)nc(-c3ccc(N(c4ccc5c6ccccc6c6cc7sc8ccccc8c7cc6c5c4)c4cccc5c4oc4ccccc45)cc3)n2)cc1 ; c1ccc2c(c1)oc1ccc(N(c3ccc4sc5ccccc5c4c3)c3ccc4c5ccccc5c5cc6sc7ccccc7c6cc5c4c3)cc12 ; c1ccc2c(c1)oc1cc3c4ccccc4c4cc(N(c5ccc(-c6cncnc6)cc5)c5cccc6c5sc5ccccc56)ccc4c3cc12]

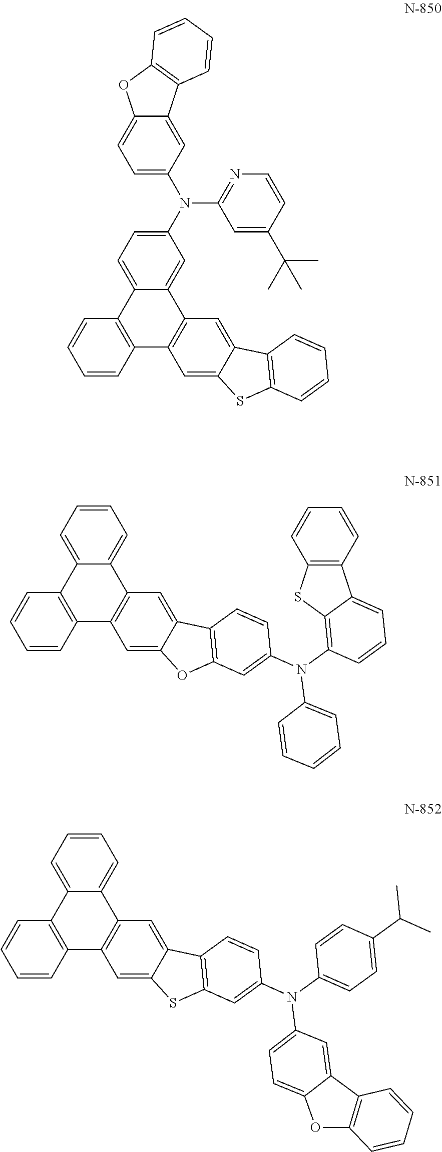 [SMILES: CC(C)(C)c1ccnc(N(c2ccc3oc4ccccc4c3c2)c2ccc3c4ccccc4c4cc5sc6ccccc6c5cc4c3c2)c1 ; c1ccc(N(c2ccc3c(c2)oc2cc4c5ccccc5c5ccccc5c4cc23)c2cccc3c2sc2ccccc23)cc1 ; CC(C)c1ccc(N(c2ccc3c(c2)sc2cc4c5ccccc5c5ccccc5c4cc23)c2ccc3oc4ccccc4c3c2)cc1]

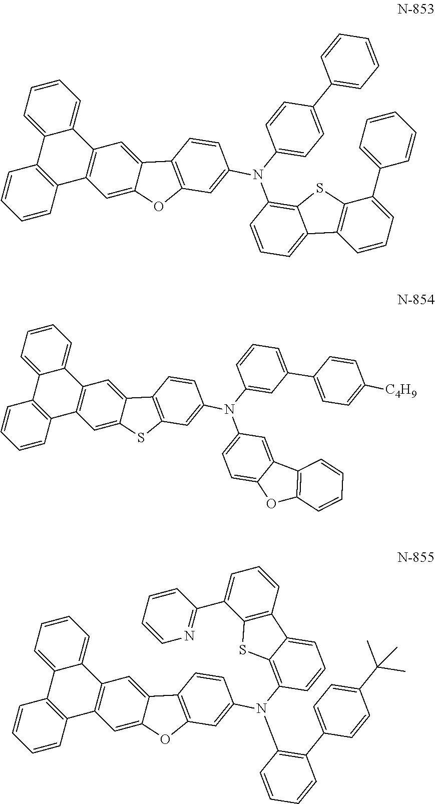

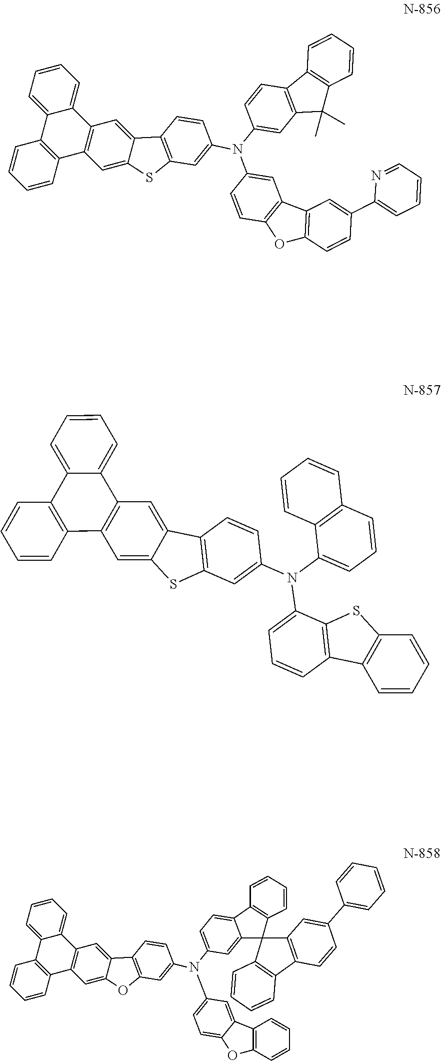 [SMILES: CC1(C)c2ccccc2-c2ccc(N(c3ccc4c(c3)sc3cc5c6ccccc6c6ccccc6c5cc34)c3ccc4oc5ccc(-c6ccccn6)cc5c4c3)cc21 ; c1ccc2c(N(c3ccc4c(c3)sc3cc5c6ccccc6c6ccccc6c5cc34)c3cccc4c3sc3ccccc34)cccc2c1 ; c1ccc(-c2ccc3c(c2)C2(c4ccccc4-3)c3ccccc3-c3ccc(N(c4ccc5c(c4)oc4cc6c7ccccc7c7ccccc7c6cc45)c4ccc5oc6ccccc6c5c4)cc32)cc1]

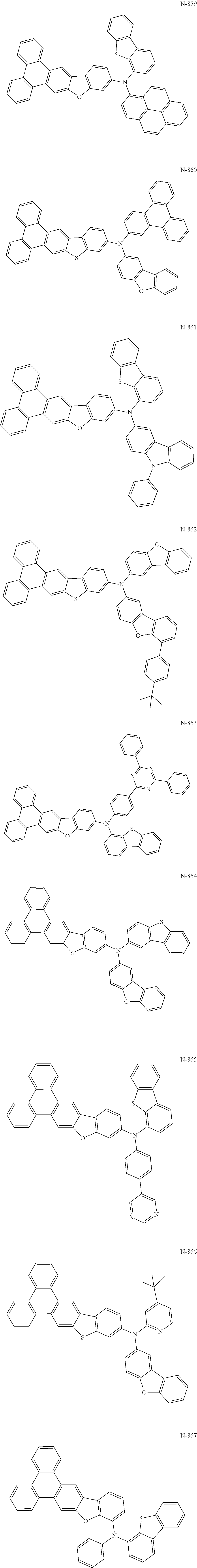

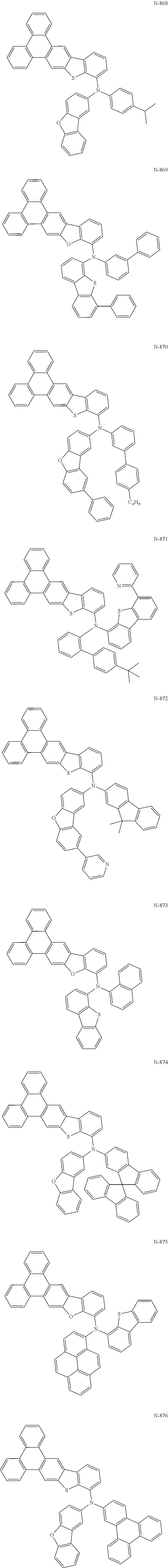

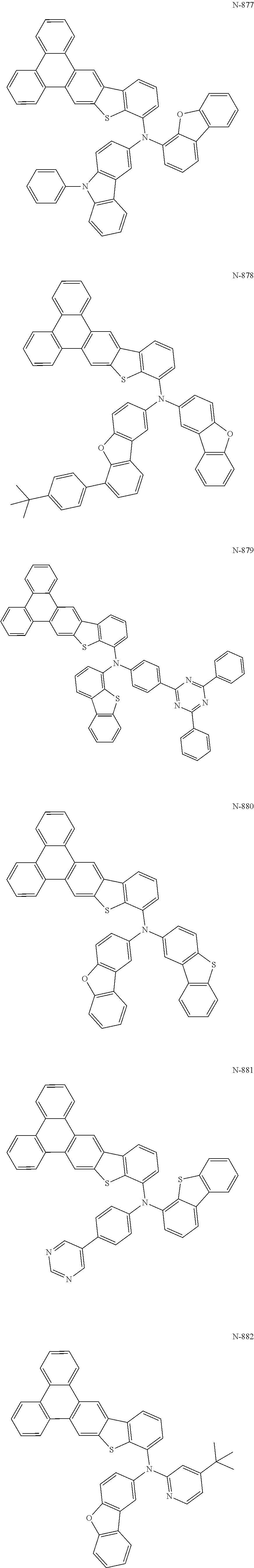

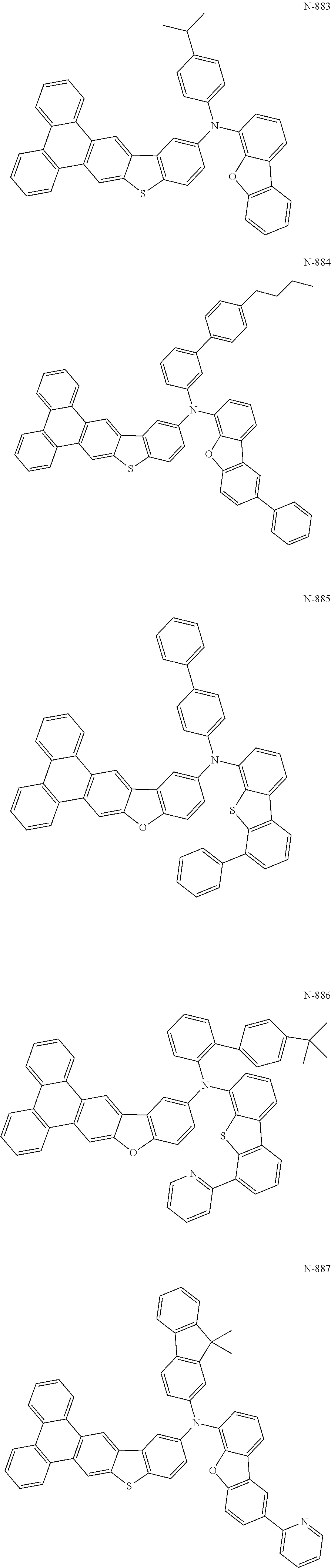 [SMILES: CC(C)c1ccc(N(c2ccc3sc4cc5c6ccccc6c6ccccc6c5cc4c3c2)c2cccc3c2oc2ccccc23)cc1 ; CCCCc1ccc(-c2cccc(N(c3ccc4sc5cc6c7ccccc7c7ccccc7c6cc5c4c3)c3cccc4c3oc3ccc(-c5ccccc5)cc34)c2)cc1 ; c1ccc(-c2ccc(N(c3ccc4oc5cc6c7ccccc7c7ccccc7c6cc5c4c3)c3cccc4c3sc3c(-c5ccccc5)cccc34)cc2)cc1 ; CC(C)(C)c1ccc(-c2ccccc2N(c2ccc3oc4cc5c6ccccc6c6ccccc6c5cc4c3c2)c2cccc3c2sc2c(-c4ccccn4)cccc23)cc1 ; CC1(C)c2ccccc2-c2ccc(N(c3ccc4sc5cc6c7ccccc7c7ccccc7c6cc5c4c3)c3cccc4c3oc3ccc(-c5ccccn5)cc34)cc21]

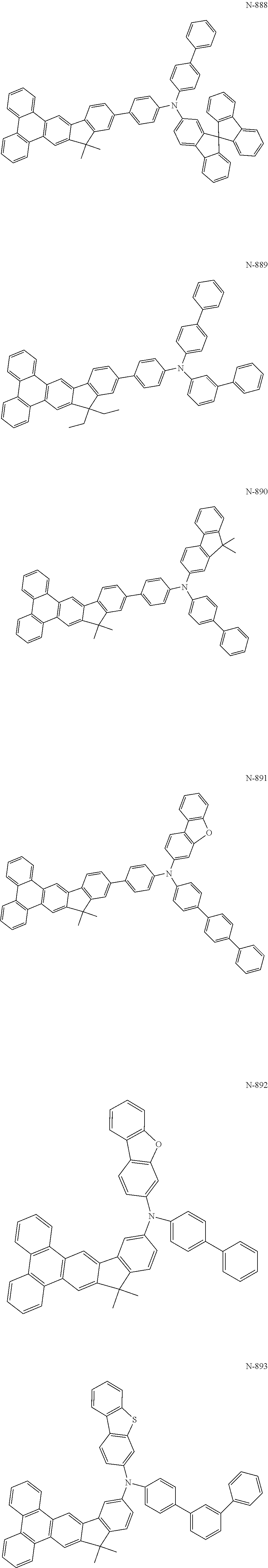

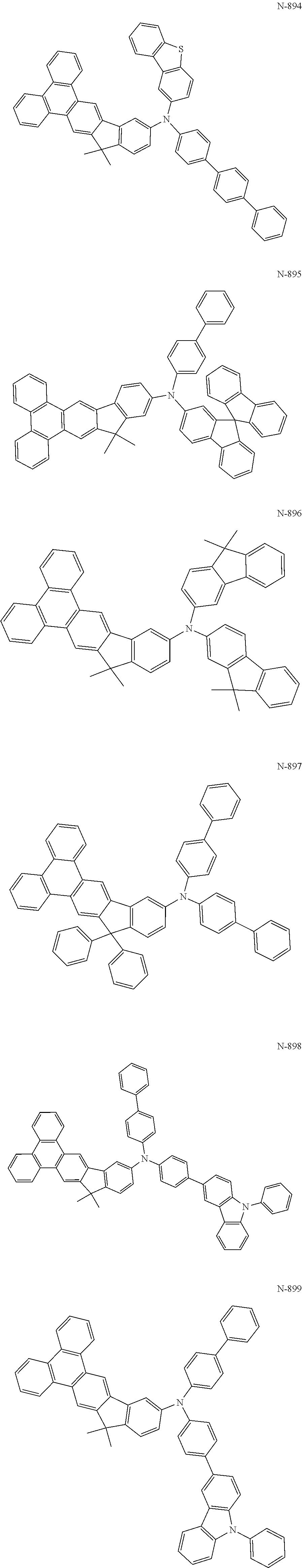

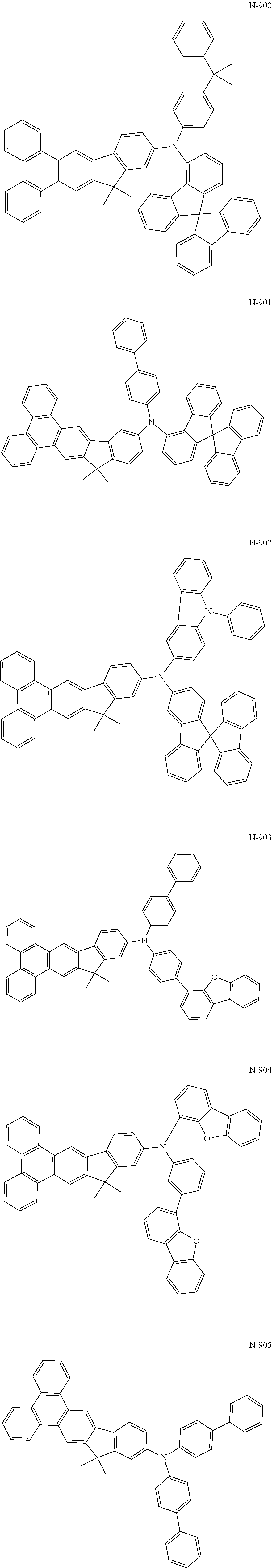

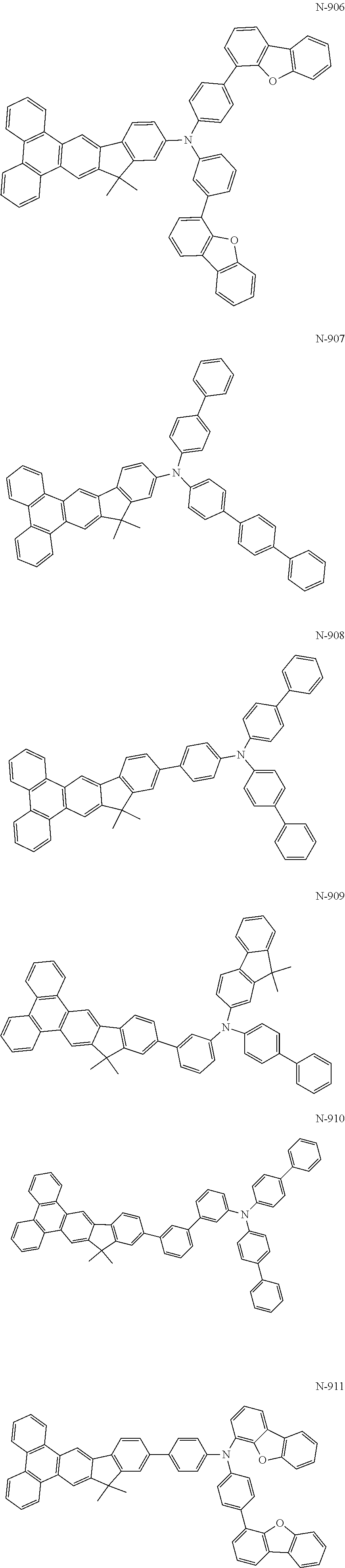

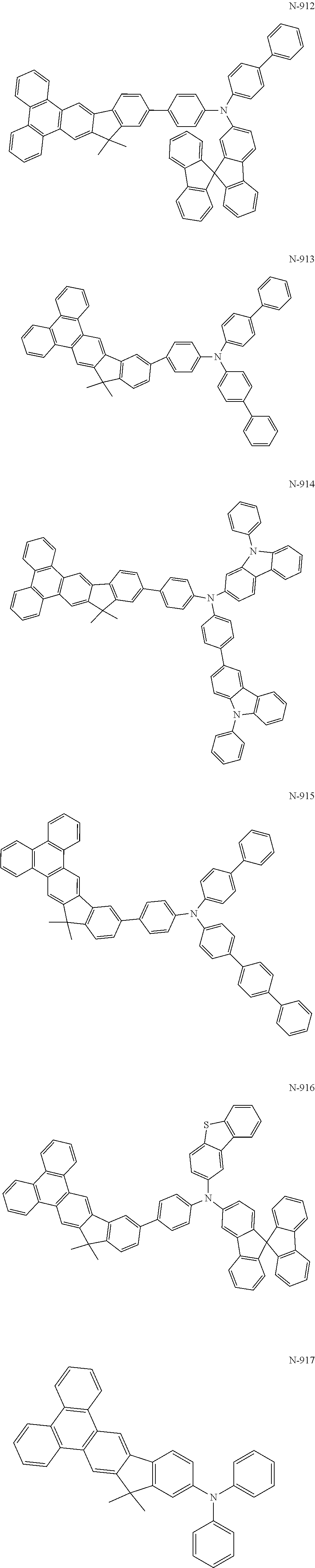

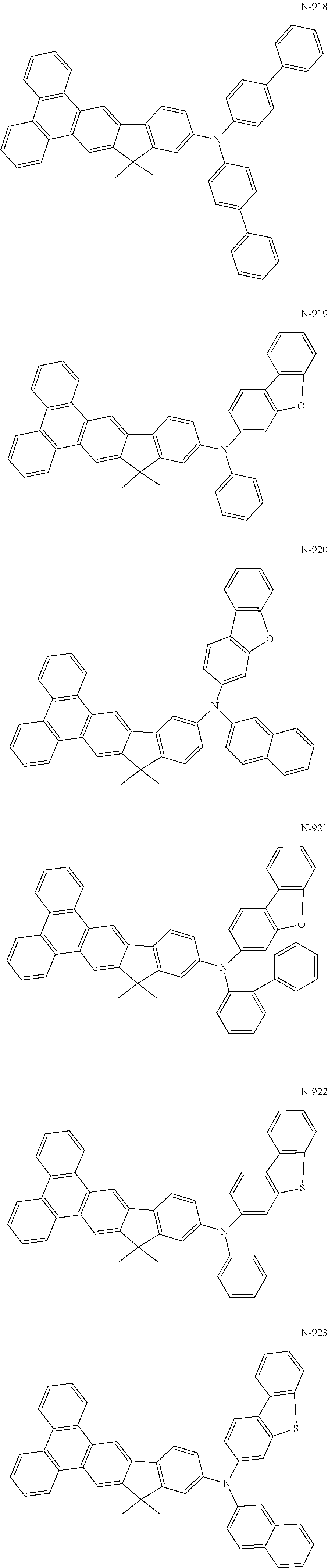

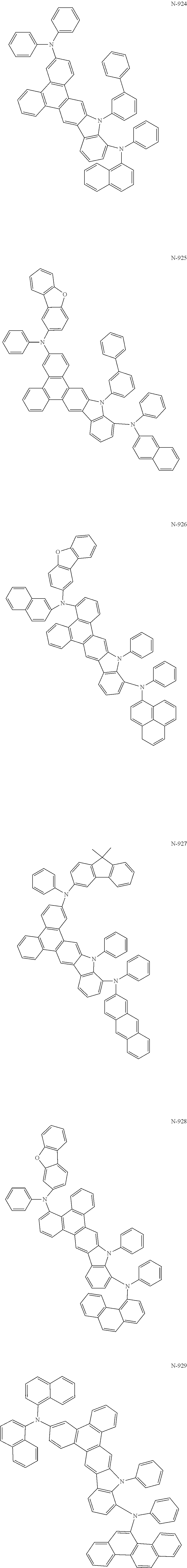

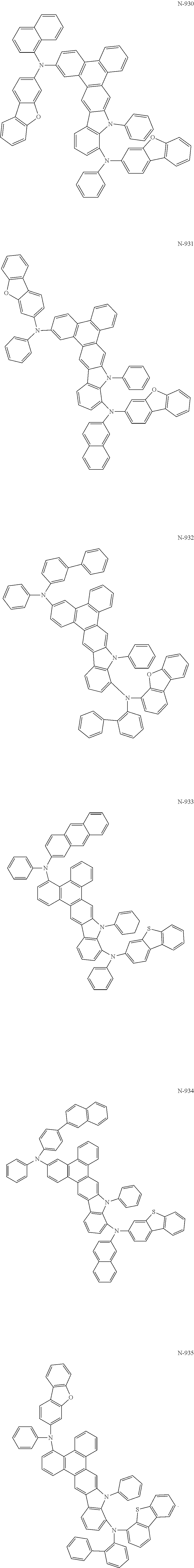

A mass ratio of compound N to compound M is from 9:1 to 1:9; preferably from 2:8 to 8:2; and more preferably from 3:7 to 7:3, and further preferably from 4:6 to 6:4.

An application of the above organic electroluminescent host material composition in an optical device is preferably an application in an organic electroluminescent device.

Preferably, the optical device includes any one of an organic electroluminescent device, an organic field effect transistor, an organic thin film transistor, an organic light emitting transistor, an organic integrated circuit, an organic solar cell, an organic field quenching device, a luminescent electrochemical cell, an organic laser diode, or an organic photoreceptor.

An organic electroluminescent material includes the above organic electroluminescent compound or the above organic electroluminescent host material composition. Preferably, the organic electroluminescent material further contains a dopant material. Preferably, the dopant material includes a phosphorescent dopant, and the phosphorescent dopant includes a complex containing transition metal.

An organic electroluminescent device includes an anode, a cathode and an organic layer arranged between the anode and the cathode; and the organic layer includes the above organic electroluminescent compound or the above organic electroluminescent host material composition.

Preferably, the organic layer includes a hole injection layer, a hole transport layer, an electron blocking layer, a light emitting layer, a hole blocking layer (electron buffer layer), an electron transport layer and an electron injection layer which are arranged in a sequentially stacked manner from an anode side to a cathode side.

An organic electroluminescent equipment includes the above organic electroluminescent device.

Unless otherwise explicitly stated, the substituents of other structures in the present disclosure are selected from one of or a combination of two of deuterium, halogen, cyano, C1-C6 alkyl, C3-C30 cycloalkyl, C6-C30 aryl, and C3-C30 heteroaryl.

The term “organic electroluminescent material” disclosed in the present disclosure means a material which may be used in an organic electroluminescent device and may contain at least one compound. If necessary, the organic electroluminescent material may be contained in any layer which constitutes the organic electroluminescent device. For example, the organic electroluminescent material may be a hole injection material, a hole transport material, a hole auxiliary material, a light emitting auxiliary material, an electron blocking material, a light emitting material (containing an organic electroluminescent host material and a dopant material), an electron buffer material, a hole blocking material, an electron transport material, an electron injection material, etc.

The organic electroluminescent material disclosed in the present disclosure may contain one organic electroluminescent material or a plurality of organic electroluminescent materials, where the plurality of organic electroluminescent materials refer to a material containing a combination of at least two organic electroluminescent materials, and the material may be contained in any layer which constitutes the organic electroluminescent device. The material may refer to both a material contained before the organic electroluminescent device (e.g., before vapor deposition) and a material contained after the organic electroluminescent device (e.g., after vapor deposition). For example, the material may be a combination of at least two compositions, and the compositions may be contained in at least one of the following: a hole injection layer, a hole transport layer, a hole auxiliary layer, a light emitting auxiliary layer, an electron blocking layer, a light emitting layer, an electron buffer layer, a hole blocking layer, an electron transport layer and an electron injection layer. The two compositions in the plurality of organic electroluminescent materials may be contained in the same layer or different layers, and may be mixed-evaporated or co-evaporated, or may be evaporated individually.

The term “organic electroluminescent host material composition” disclosed in the present disclosure refers to an organic electroluminescent material containing a combination of at least two host materials. The composition may refer to both a material contained before the organic electroluminescent device (e.g., before vapor deposition) and a material contained after the organic electroluminescent device (e.g., after vapor deposition). The composition disclosed in the present disclosure may be contained in any light emitting layer which constitutes the organic electroluminescent device. Two or more compounds contained in the plurality of host materials in the composition disclosed in the present disclosure may be contained in one light emitting layer or may be contained in different light emitting layers respectively. For example: when one layer contains two or more host materials, the layer may be formed through mixed evaporation or may be formed through individual co-evaporation.

The “halogen” in the present disclosure may include fluorine, chlorine, bromine or iodine.

The “C1-C30 alkyl” in the present disclosure refers to a univalent substituent derived from linear or branched saturated hydrocarbon with 1 to 30 carbon atoms, and instances of the univalent substituent include but are not limited to methyl, ethyl, n-propyl, isopropyl, n-butyl, isobutyl, sec-butyl, tert-butyl, pentyl, isopentyl and hexyl.

The “C3-C30 cycloalkyl” in the present disclosure refers to monocyclic hydrocarbon or polycyclic hydrocarbon with 1 to 30 carbon atoms around a main chain, and cycloalkane may include cyclopropyl, cyclobutyl, cyclopentyl, cyclohexyl, cyclopentylmethyl, cyclohexylmethyl, adamantyl, etc.

The aryl and arylenyl in the present disclosure include monocyclic, polycyclic or fused-ring aryl, and rings may be interrupted by short non-aromatic units and may contain spiro structures, including but not limited to phenyl, biphenyl, triphenyl, naphthyl, phenanthryl, phenylphenanthryl, binaphthyl, phenylnaphthyl, naphthylphenyl, anthryl, indenyl, triphenylenyl, tetracenyl, pyrenyl, perylenyl, fluorenyl, phenylfluorenyl, diphenylfluorenyl, benzofluorenyl, spirobifluorenyl, chrysenyl, naphthonaphthyl, fluoranthenyl, etc.

The heteroaryl and heteroarylenyl in the present disclosure include monocyclic, polycyclic or fused-ring heteroaryl, rings may be interrupted by short non-aromatic units, and heteroatoms include nitrogen, oxygen and sulfur. The heteroaryl and heteroarylenyl include but are not limited to furyl, thienyl, pyrryl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, benzofuryl, benzothienyl, isobenzofuryl, dibenzofuryl, dibenzothienyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenothiazinyl, phenanthridinyl, benzodioxolyl, dihydroacridinyl, derivatives thereof, etc.

The “substituted” in the present disclosure refers to the substitution of a hydrogen atom in a compound by another substituent. The position is not limited to a specific position, as long as hydrogen at that position can be substituted by a substituent. It also includes the substitution of the hydrogen atom by a group formed by the connection of two or more substituents. When two or more substituents appear, they can be the same or different. For example, the group formed by the connection of the two or more substituents may be pyridine-triazine. That is, pyridine-triazine may be interpreted as a heteroaryl substituent, or a substituent where two heteroaryl substituents are connected.

In the present disclosure, unless otherwise specified, hydrogen atoms include protium, deuterium, and tritium.

The groups of the present disclosure limit the range of carbon atom numbers, which should be any integer within the limited range, for example, C6-C30 aryl represents that the carbon atom number of aryl may be any integer within the range of 6-30, such as 6, 8, 10, 13, 15, 17, 20, 22, 25, or 30.

When the groups in the present disclosure have substituents, the substituents are each independently selected from deuterium, halogen, cyano, nitryl, unsubstituted or R′ substituted C1-C4 linear or branched alkyl, unsubstituted or R′ substituted C6-C20 aryl, unsubstituted or R′ substituted C3-C20 heteroaryl, and unsubstituted or R′ substituted C6-C20 arylamine group; and R′ is selected from deuterium, halogen, cyano and nitryl.

A specific combination containing the compound of the present disclosure is used as the host material composition, which may provide the organic electroluminescent device having improved light emitting efficiency and service life characteristics compared to a conventional organic electroluminescent device, and manufacture a display system or illuminating system using the organic electroluminescent device.

The technical solution of the present disclosure has the following advantages:

1. The organic electroluminescent compound provided by the present disclosure is the compound M having the structure shown in Formula (2), the compound M has obviously more excellent performance compared to CBP, REF-1, etc. disclosed in the prior art, and a device prepared from the compound can have a lower lighting voltage, i.e., a lower driving voltage. The compound M may match with a compound containing a polycyclic heteromatic group of triphenylene, namely the compound N in the present disclosure to be used as an organic electroluminescent host material of an organic light emitting device, and under the synergistic effect, a lighting voltage of the organic light emitting device can be lowered obviously, the light emitting efficiency is improved remarkably, and the service life is prolonged remarkably. Therefore, the compound M having the structure shown in Formula (2) in the present disclosure may match with the compound N to achieve the advantage of remarkably improving the performance of the light emitting host material, and the compound M can be used for producing an organic electroluminescent device with the characteristics of high light emitting efficiency and long service life.

2. The organic electroluminescent host material composition provided by the present disclosure includes the compound M having the structure shown in Formula (2) and the compound N having the structure shown in Formula (1); after a light emitting host material formed by mutual matching of the compound M and the compound N is applied to a light emitting material to prepare light emitting equipment, the compound M and the compound N can improve the effect synergistically and match with each other to obviously lower a lighting voltage, remarkably improve the light emitting efficiency and remarkably prolong the service life of the light emitting equipment; and the effects of obviously improving the light emitting efficiency of the light emitting equipment and remarkably prolonging the service life of the light emitting equipment are achieved. In the present disclosure, through mutual matching of the compound M having the structure shown in Formula (2) and the compound N having the structure shown in Formula (1), when the compounds are applied to a light emitting device, the light emitting device is enabled to have a low driving voltage (3.44 V or below), high current efficiency (24 Cd/A or above) and long service life (260 h or above).

### DETAILED DESCRIPTION

The following examples are provided for a better understanding of the present disclosure and are not limited to the preferred embodiments, they do not limit the content and scope of protection of the present disclosure, and any product that is the same as or similar to the present disclosure obtained by anyone under the inspiration of the present disclosure or by combining the present disclosure with other prior art features falls within the scope of protection of the present disclosure.

If specific experimental steps or conditions are not specified in the examples, the operation or conditions of conventional experimental steps described in the literature in this field can be carried out. The adopted reagents or instruments which are not specified with the manufacturer are conventional commercially-available reagent products.

### Example 1

An organic electroluminescent compound is a compound M-17, and its synthesis process is as follows:

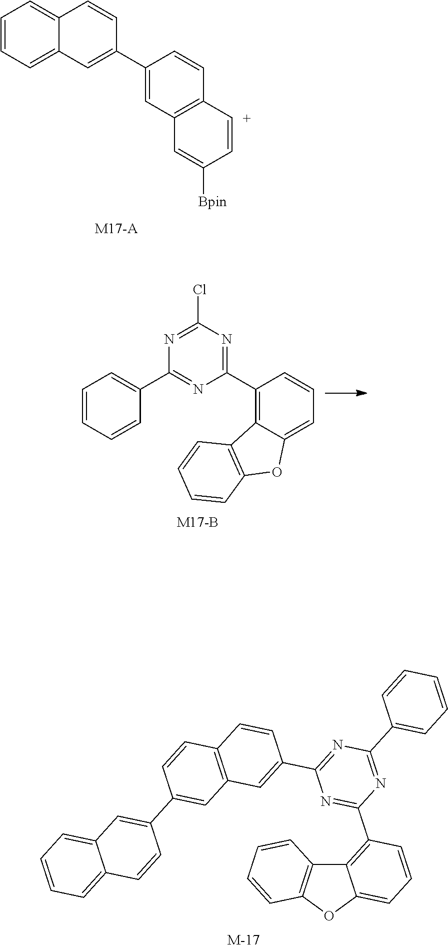

A 50 mL double-neck round-bottom flask was taken, a stirrer and an upwards-connected return pipe were placed in the flask, the flask was filled with nitrogen after being dried, a compound M17-A (19.8 mmol, CAS: 1884145-03-2), M17-B (20.75 mmol, CAS: 1883265-32-4), tetrakis(triphenylphosphine)palladium (0.396 mmol), potassium carbonate (39.6 mmol), 35 mL of toluene, 15 mL of ethanol and 15 mL of distilled water were added, and a mixture was stirred at 90 degrees Celsius for 8 hours. After a reaction was completed, the mixture was added into methanol dropwise, and an obtained solid was filtered. The obtained solid was purified through column chromatography to obtain the compound M-17 (8.5 g, yield: 75%).

Elemental analysis: C₄₁H₂₅N₃O; theoretical value: C, 85.54; H, 4.38; N, 7.30; O, 2.78; measured value: C, 85.52; H, 4.38; N, 7.32; HRMS (ESI) m/z (M+): theoretical value: 575.20; measured value: 576.34.

### Example 2

An organic electroluminescent compound is a compound M-296, and its synthesis process is as follows:

(I) Synthesis of an intermediate M296-A, a synthetic route being as follows:

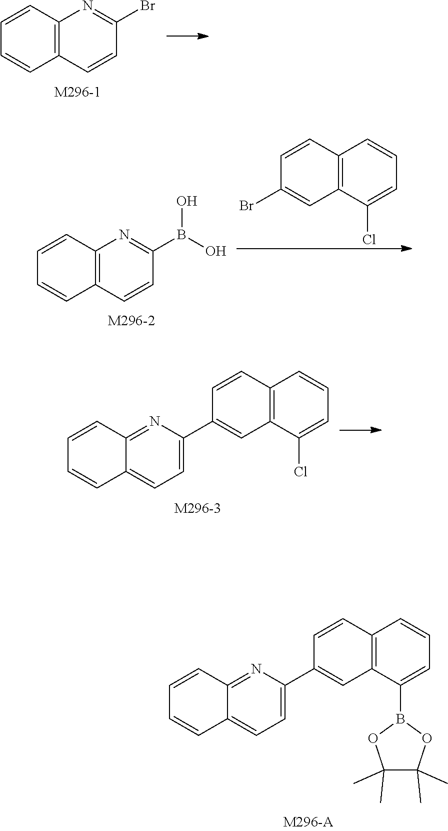 [SMILES: Brc1ccc2ccccc2n1 ; OB(O)c1ccc2ccccc2n1 ; Clc1cccc2ccc(Br)cc12 ; Clc1cccc2ccc(-c3ccc4ccccc4n3)cc12 ; CC1(C)OB(c2cccc3ccc(-c4ccc5ccccc5n4)cc23)OC1(C)C]

An intermediate M296-1 (2-bromoquinoline, CAS: 2005-43-8, 20 g) and 200 mL of anhydrous tetrahydrofuran were added into a 250 mL three-necked flask with a thermometer and a magnetic stirrer, the temperature was reduced to −78° C. under a nitrogen protection condition, n-butyl lithium (1.6 M, 45.2 mL) was dropwise added with the temperature controlled, stirring was conducted for 1 h after dropwise adding, triisopropyl borate (19.52 g) was dropwise added with the temperature controlled at −78° C., a mixture was transferred to the room temperature after dropwise adding to react for 12 h, a hydrochloric acid solution (6.5 mL of 36% hydrochloric acid +24 mL of water) was dropwise added, 50 mL of ethyl acetate and 25 mL of water were added into a reaction liquid for extraction and liquid separation, an organic phase was spin-dried and then 50 mL of n-hexane was added, reflux beating was conducted for 1 h, filtering was conducted at the room temperature, and 15 g of an intermediate M296-2 was obtained after drying.

The intermediate M296-2 (15 g), an intermediate 7-bromo-1-chloronaphthalene (21.9 g), potassium carbonate (16.6 g) and tetrakis(triphenylphosphine)palladium (2.0 g) were added into a 250 mL three-necked flask with a thermometer and a magnetic stirrer, toluene (80 mL), ethanol (35 mL) and water (35 mL) were added, under a nitrogen protection condition, the temperature was raised to 85° C. to react for 6 h, 50 mL of ethyl acetate and 25 mL of water were added into a reaction liquid for extraction and liquid separation, an organic phase was subjected to sample mixing and column chromatography, and 15 g of an intermediate M296-3 was obtained.

The intermediate M296-3 (15 g), bis(pinacolato)diboron (15.8 g), potassium acetate (10 g) and Pd(dppf)Cl₂ (0.64 g) were added into a 250 mL three-necked flask with a thermometer and a magnetic stirrer, 1,4-dioxane (150 mL) was added, under a nitrogen protection condition, the temperature was raised to 110° C. to react for 4 h, 100 mL of toluene and 100 mL of water were added into a reaction liquid for extraction and liquid separation, an organic phase was subjected to sample mixing and column chromatography, and 16 g of the intermediate M296-A was obtained.

(II) Synthesis of the compound M-296, a synthetic route being as follows:

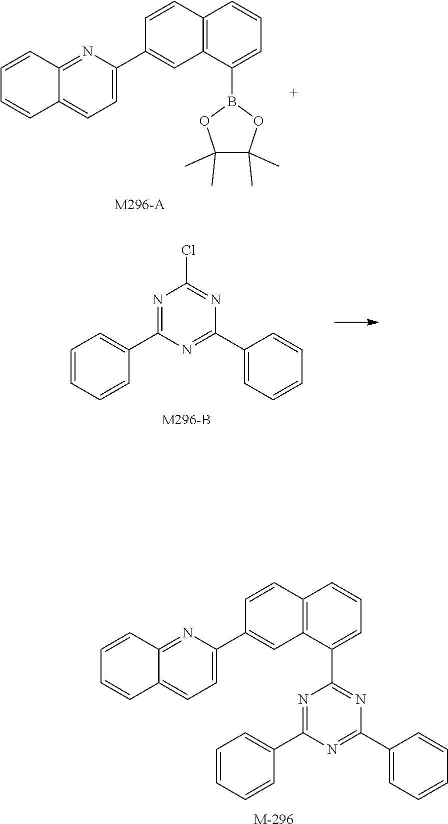

The intermediate M296-A (16 g), an intermediate M296-B (2-chloro-4,6-diphenyl-1,3,5-triazine, CAS: 3842-55-5, 11.2 g), potassium carbonate (11.6 g) and tetrakis(triphenylphosphine)palladium (1.3 g) were added into a 250 mL three-necked flask with a thermometer and a magnetic stirrer, toluene (110 mL), ethanol (50 mL) and water (50 mL) were added, under a nitrogen protection condition, the temperature was raised to 85° C. to react for 6 h, water and ethanol were added into a reaction liquid at the room temperature for filtration, and the product M-296 was obtained after drying, 16 g (yield 78%).

Elemental analysis: C₃₄H₂₂N₄; theoretical value: C, 83.93; H, 4.56; N, 11.51; measured value: C, 83.95; H, 4.56; N, 11.49; HRMS (ESI) m/z (M+): theoretical value: 486.18; measured value: 487.12.

### Example 3

An organic electroluminescent compound is a compound M-381, and its synthesis process is as follows:

(I) Synthesis of an intermediate M381-B, a synthetic route being as follows:

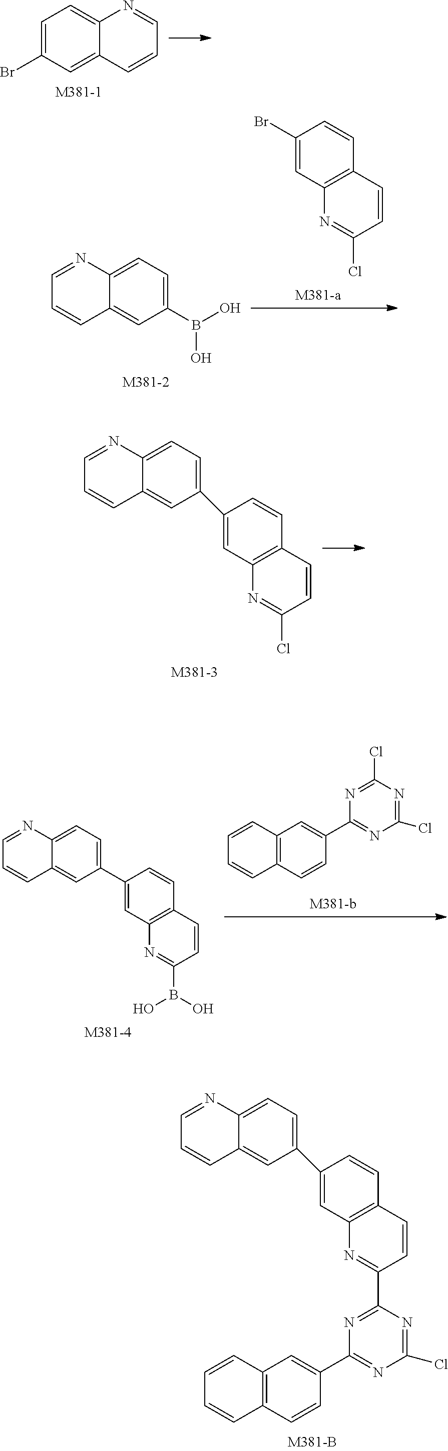

An intermediate M381-1 (CAS: 5332-25-2, 20 g) and 200 mL of anhydrous tetrahydrofuran were added into a 250 mL three-necked flask with a thermometer and a magnetic stirrer, the temperature was reduced to −78° C. under a nitrogen protection condition, n-butyl lithium (1.6 M, 45.2 mL) was dropwise added with the temperature controlled, stirring was conducted for 1 h after dropwise adding, triisopropyl borate (19.52 g) was dropwise added with the temperature controlled at −78° C., a mixture was transferred to the room temperature after dropwise adding to react for 12 h, a hydrochloric acid solution (6.5 mL of 36% hydrochloric acid+24 mL of water) was dropwise added, 50 mL of ethyl acetate and 25 mL of water were added into a reaction liquid for extraction and liquid separation, an organic phase was spin-dried and then 50 mL of n-hexane was added, reflux beating was conducted for 1 h, filtering was conducted at the room temperature, and 15 g of an intermediate M381-2 was obtained after drying.

The intermediate M381-2 (15 g), a raw material M367-a (CAS: 99455-15-9, 21 g), potassium carbonate (16.6 g) and tetrakis(triphenylphosphine)palladium (2.0 g) were added into a 250 mL three-necked flask with a thermometer and a magnetic stirrer, toluene (80 mL), ethanol (35 mL) and water (35 mL) were added, under a nitrogen protection condition, the temperature was raised to 85° C. to react for 6 h, 50 mL of ethyl acetate and 25 mL of water were added into a reaction liquid for extraction and liquid separation, an organic phase was subjected to sample mixing and column chromatography, and 13 g of an intermediate M381-3 was obtained.

The intermediate M381-3 (13 g) and 200 mL of anhydrous tetrahydrofuran were added into a 250 mL three-necked flask with a thermometer and a magnetic stirrer, the temperature was reduced to −78° C. under a nitrogen protection condition, n-butyl lithium (1.6 M, 40 mL) was dropwise added with the temperature controlled, stirring was conducted for 1 h after dropwise adding, triisopropyl borate (17 g) was dropwise added with the temperature controlled at −78° C., a mixture was transferred to the room temperature after dropwise adding to react for 12 h, a hydrochloric acid solution (6.5 mL of 36% hydrochloric acid+24 mL of water) was dropwise added, 50 mL of ethyl acetate and 25 mL of water were added into a reaction liquid for extraction and liquid separation, an organic phase was spin-dried and then 50 mL of n-hexane was added, reflux beating was conducted for 1 h, filtering was conducted at the room temperature, and 12 g of an intermediate M381-4 was obtained after drying.

The intermediate M381-4 (12 g), a raw material M381-b (CAS: 112719-97-8, 11 g), potassium carbonate (11 g) and tetrakis(triphenylphosphine)palladium (1.2 g) were added into a 250 mL three-necked flask with a thermometer and a magnetic stirrer, toluene (80 mL), ethanol (35 mL) and water (35 mL) were added, under a nitrogen protection condition, the temperature was raised to 85° C. to react for 6 h, 50 mL of ethyl acetate and 25 mL of water were added into a reaction liquid for extraction and liquid separation, an organic phase was subjected to sample mixing and column chromatography, and 16 g of the intermediate M381-B was obtained.

(II) Synthesis of the compound M-381, a synthetic route being as follows:

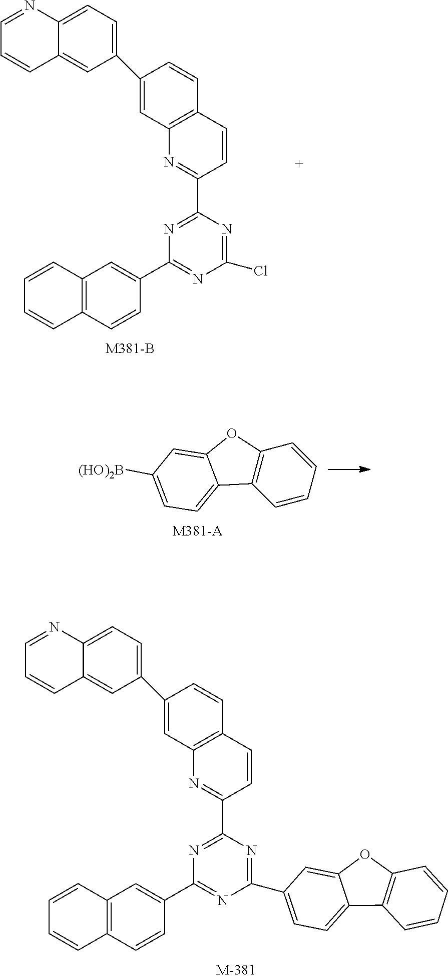 [SMILES: Clc1nc(-c2ccc3ccccc3c2)nc(-c2ccc3ccc(-c4ccc5ncccc5c4)cc3n2)n1 ; OB(O)c1ccc2c(c1)oc1ccccc12 ; c1ccc2cc(-c3nc(-c4ccc5c(c4)oc4ccccc45)nc(-c4ccc5ccc(-c6ccc7ncccc7c6)cc5n4)n3)ccc2c1]

The intermediate M381-B (16 g), a raw material M381-A (6.85 g, CAS: 395087-89-5), potassium carbonate (9 g) and tetrakis(triphenylphosphine)palladium (1.0 g) were added into a 250 mL three-necked flask with a thermometer and a magnetic stirrer, toluene (80 mL), ethanol (35 mL) and water (35 mL) were added, under a nitrogen protection condition, the temperature was raised to 85° C. to react for 6 h, 50 mL of ethyl acetate and 25 mL of water were added into a reaction liquid for extraction and liquid separation, an organic phase was subjected to sample mixing and column chromatography, and the final product M-381 was obtained, 15 g (yield 74%).

Elemental analysis: C₄₃H₂₅N₅₀; theoretical value: C, 82.28; H, 4.01; N, 11.16; O, 2.55; measured value: C, 82.30; H, 4.01; N, 11.14; HRMS (ESI) m/z (M+): theoretical value: 627.21; measured value: 628.13.

### Example 4

An organic electroluminescent compound is a compound M-76, M-108, M-145, M-253, M-308, M-365 or M-371, and a synthesis process of the above compounds M is as follows:

Toluene (80 mL), ethanol (35 mL) and water (35 mL) were added into a raw material Mn-B (16 g), a raw material Mn-A (6.85 g), potassium carbonate (9 g) and tetrakis(triphenylphosphine)palladium (1.0 g), under a nitrogen protection condition, the temperature was raised to 85° C. to react for 6 h, 50 mL of ethyl acetate and 25 mL of water were added into a reaction liquid for extraction and liquid separation, an organic phase was subjected to sample mixing and column chromatography, and a final product was obtained.

The structures and yields of the raw material Mn-B, the raw material Mn-A and the product were as shown in Table 1 below. The elemental analysis results of the prepared compounds were as shown in Table 2.

TABLE 1

| Raw material Mn-A | Raw material Mn-B |
|---|---|
| 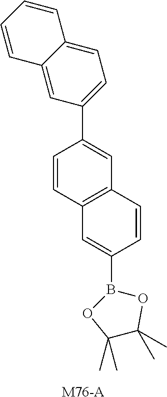 | 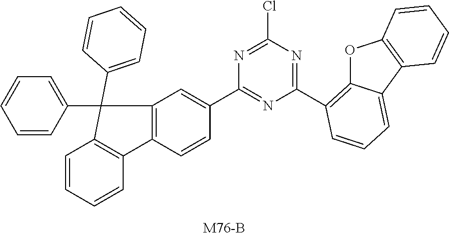 [SMILES: Clc1nc(-c2ccc3c(c2)C(c2ccccc2)(c2ccccc2)c2ccccc2-3)nc(-c2cccc3c2oc2ccccc23)n1] |
| 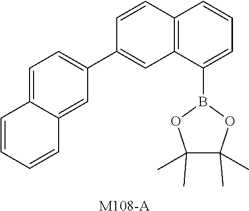 | 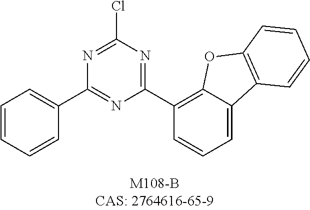 [SMILES: Clc1nc(-c2ccccc2)nc(-c2cccc3c2oc2ccccc23)n1] |
| 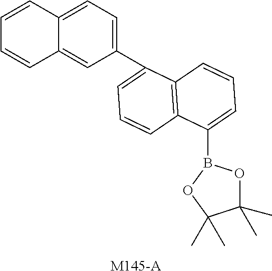 | 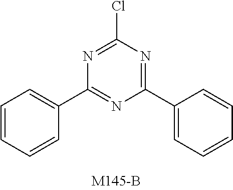 [SMILES: Clc1nc(-c2ccccc2)nc(-c2ccccc2)n1] |
| 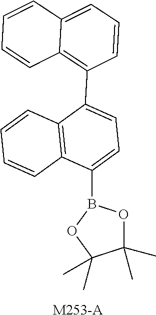 | 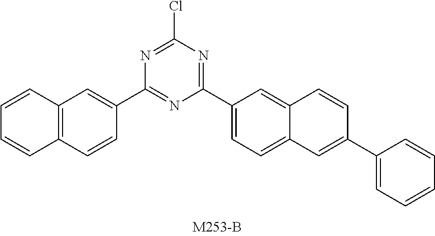 [SMILES: Clc1nc(-c2ccc3ccccc3c2)nc(-c2ccc3cc(-c4ccccc4)ccc3c2)n1] |
| 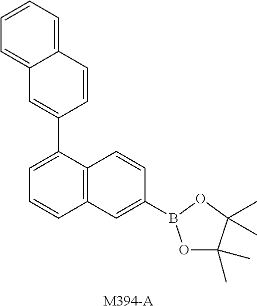 | 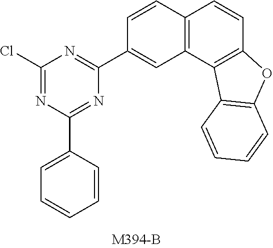 [SMILES: Clc1nc(-c2ccccc2)nc(-c2ccc3ccc4oc5ccccc5c4c3c2)n1] |
| 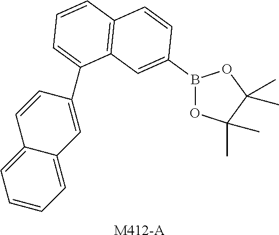 | 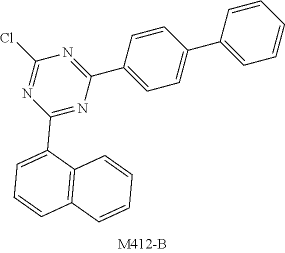 [SMILES: Clc1nc(-c2ccc(-c3ccccc3)cc2)nc(-c2cccc3ccccc23)n1] |
| 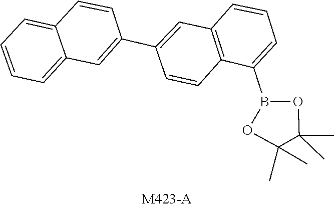 | 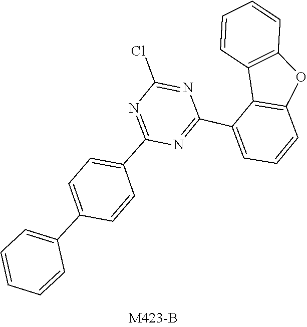 [SMILES: Clc1nc(-c2ccc(-c3ccccc3)cc2)nc(-c2cccc3oc4ccccc4c23)n1] |
| 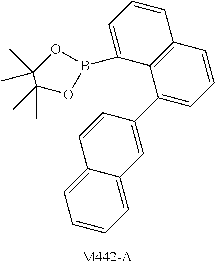 | 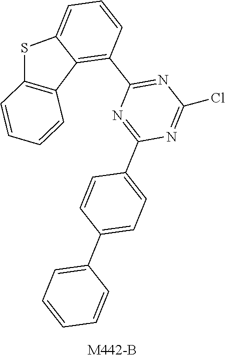 [SMILES: Clc1nc(-c2ccc(-c3ccccc3)cc2)nc(-c2cccc3sc4ccccc4c23)n1] |
| 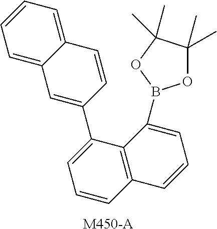 | 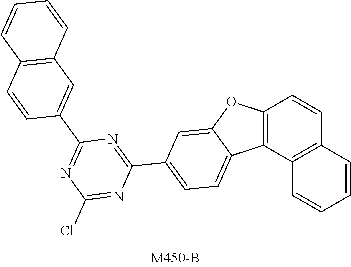 [SMILES: Clc1nc(-c2ccc3ccccc3c2)nc(-c2ccc3c(c2)oc2ccc4ccccc4c23)n1] |
| 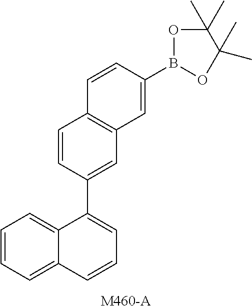 | 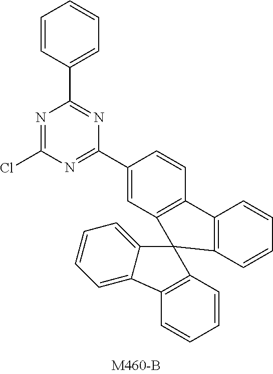 [SMILES: Clc1nc(-c2ccccc2)nc(-c2ccc3c(c2)C2(c4ccccc4-c4ccccc42)c2ccccc2-3)n1] |
| 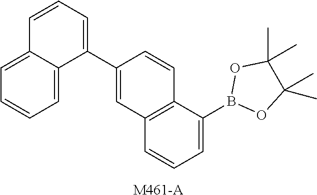 | 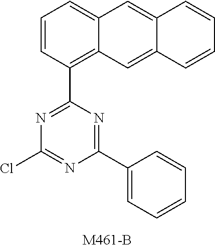 [SMILES: Clc1nc(-c2ccccc2)nc(-c2cccc3cc4ccccc4cc23)n1] |
| 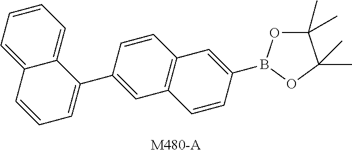 | 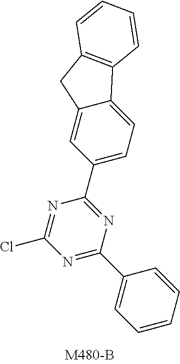 [SMILES: Clc1nc(-c2ccccc2)nc(-c2ccc3c(c2)Cc2ccccc2-3)n1] |
| 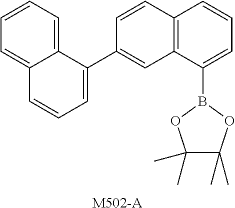 | 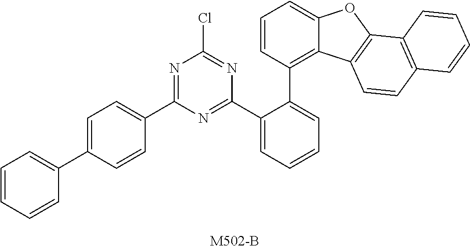 [SMILES: Clc1nc(-c2ccc(-c3ccccc3)cc2)nc(-c2ccccc2-c2cccc3oc4c5ccccc5ccc4c23)n1] |
| 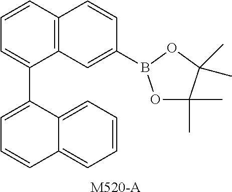 | 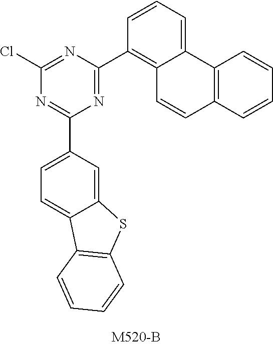 [SMILES: Clc1nc(-c2ccc3c(c2)sc2ccccc23)nc(-c2cccc3c2ccc2ccccc23)n1] |
| 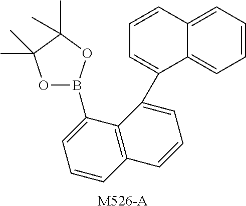 | 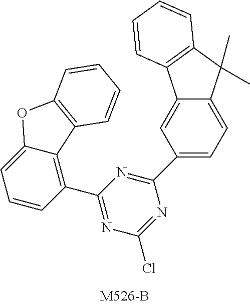 [SMILES: CC1(C)c2ccccc2-c2cc(-c3nc(Cl)nc(-c4cccc5oc6ccccc6c45)n3)ccc21] |
| 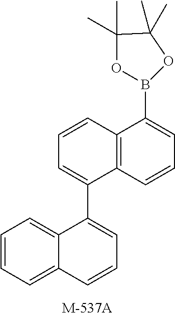 | 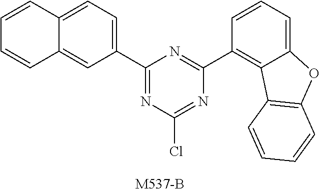 [SMILES: Clc1nc(-c2ccc3ccccc3c2)nc(-c2cccc3oc4ccccc4c23)n1] |
| 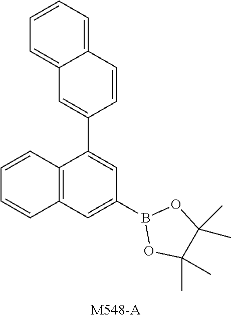 | 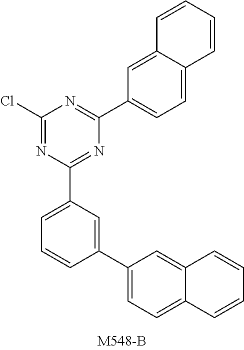 [SMILES: Clc1nc(-c2cccc(-c3ccc4ccccc4c3)c2)nc(-c2ccc3ccccc3c2)n1] |
| 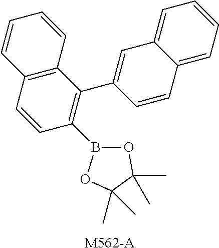 | 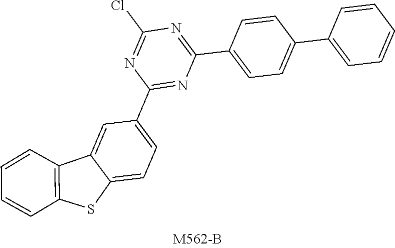 [SMILES: Clc1nc(-c2ccc(-c3ccccc3)cc2)nc(-c2ccc3sc4ccccc4c3c2)n1] |
| 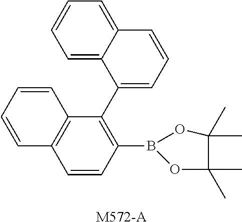 | 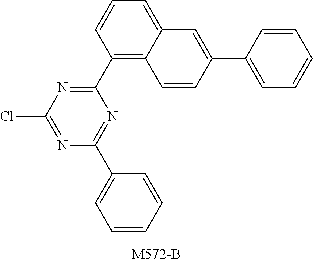 [SMILES: Clc1nc(-c2ccccc2)nc(-c2cccc3cc(-c4ccccc4)ccc23)n1] |
| 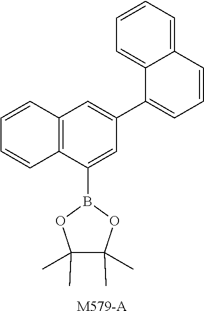 | 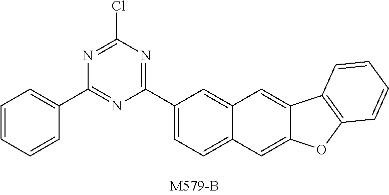 [SMILES: Clc1nc(-c2ccccc2)nc(-c2ccc3cc4oc5ccccc5c4cc3c2)n1] |
| 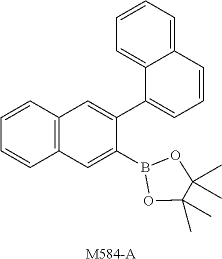 | 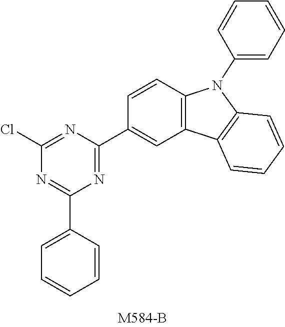 [SMILES: Clc1nc(-c2ccccc2)nc(-c2ccc3c(c2)c2ccccc2n3-c2ccccc2)n1] |
| 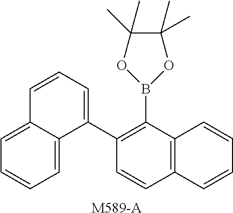 | 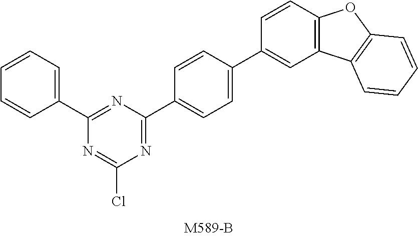 [SMILES: Clc1nc(-c2ccccc2)nc(-c2ccc(-c3ccc4oc5ccccc5c4c3)cc2)n1] |
| 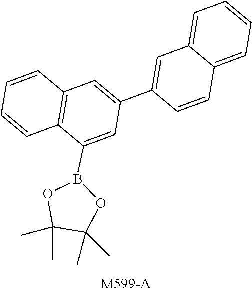 | 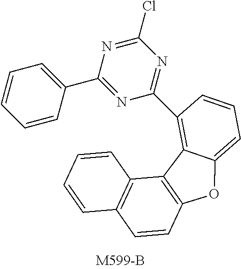 [SMILES: Clc1nc(-c2ccccc2)nc(-c2cccc3oc4ccc5ccccc5c4c23)n1] |
| 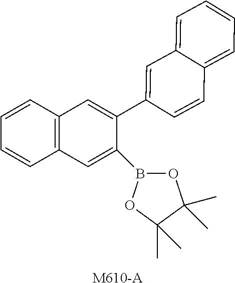 | 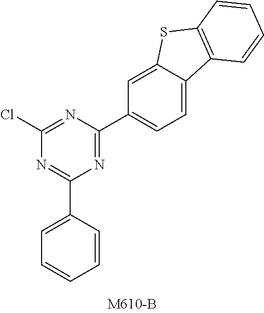 [SMILES: Clc1nc(-c2ccccc2)nc(-c2ccc3c(c2)sc2ccccc23)n1] |
| 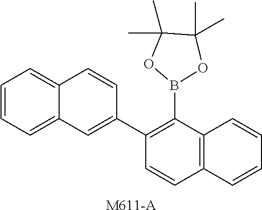 | 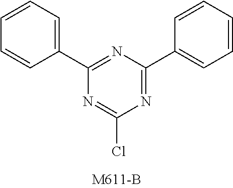 [SMILES: Clc1nc(-c2ccccc2)nc(-c2ccccc2)n1] |
| 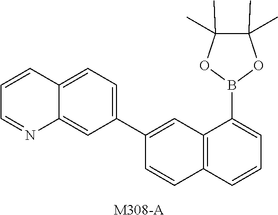 | 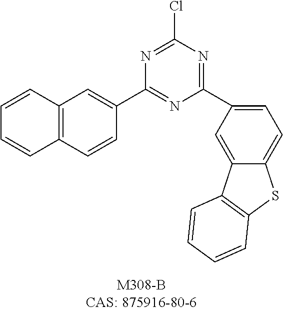 [SMILES: Clc1nc(-c2ccc3ccccc3c2)nc(-c2ccc3sc4ccccc4c3c2)n1] |
| 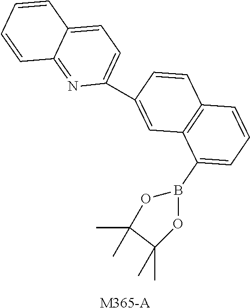 | 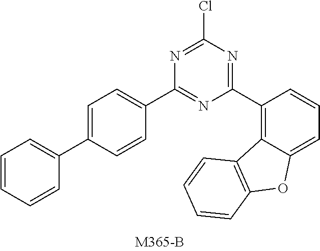 [SMILES: Clc1nc(-c2ccc(-c3ccccc3)cc2)nc(-c2cccc3oc4ccccc4c23)n1] |
| 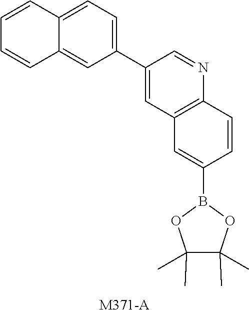 | 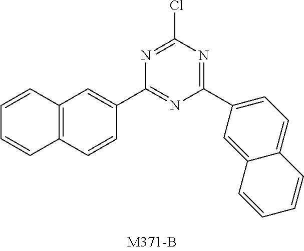 [SMILES: Clc1nc(-c2ccc3ccccc3c2)nc(-c2ccc3ccccc3c2)n1] |

| Product M-n | Yield % |
|---|---|
| 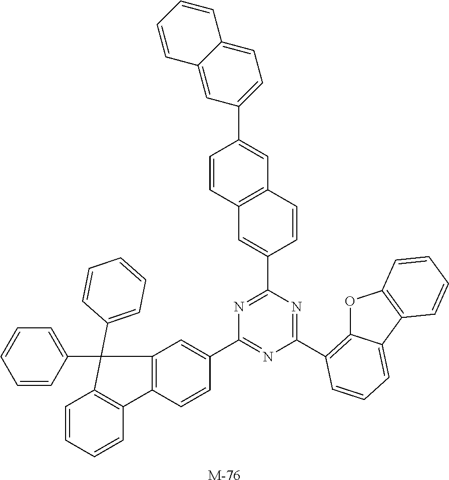 [SMILES: c1ccc(C2(c3ccccc3)c3ccccc3-c3ccc(-c4nc(-c5ccc6cc(-c7ccc8ccccc8c7)ccc6c5)nc(-c5cccc6c5oc5ccccc56)n4)cc32)cc1 ; "M-76"] | 76 |
| 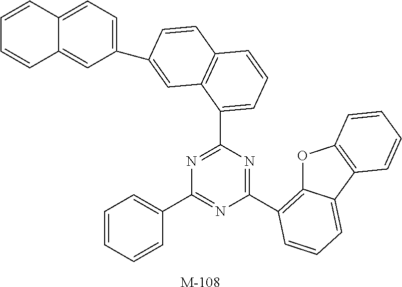 | 83 |
| 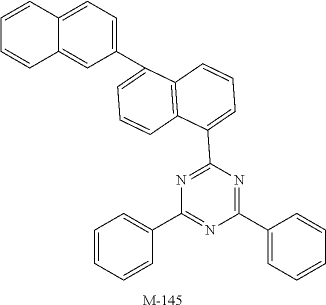 | 80 |
| 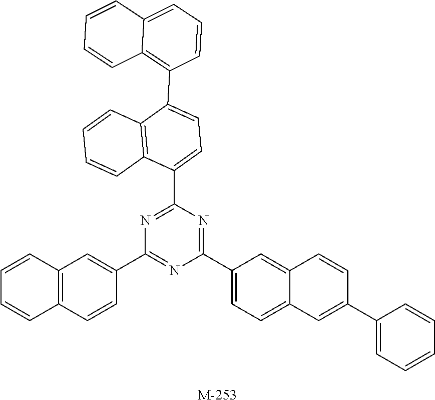 [SMILES: c1ccc(-c2ccc3cc(-c4nc(-c5ccc6ccccc6c5)nc(-c5ccc(-c6cccc7ccccc67)c6ccccc56)n4)ccc3c2)cc1 ; "M-253"] | 77 |
| 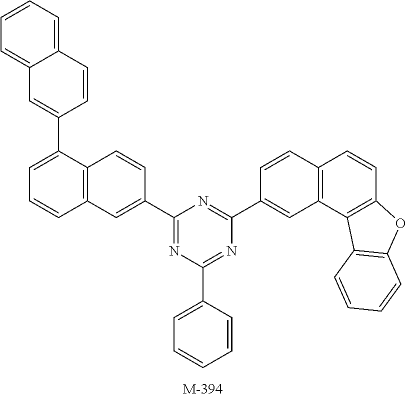 | 79 |
| 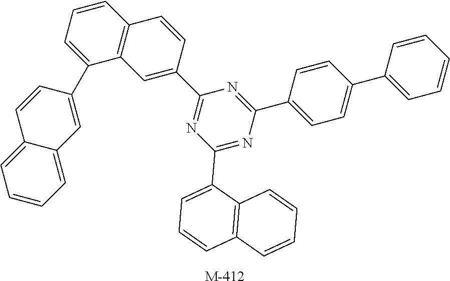 | 74 |
| 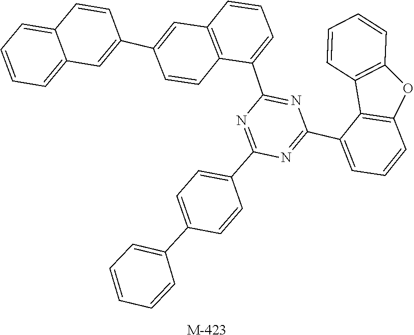 [SMILES: c1ccc(-c2ccc(-c3nc(-c4cccc5cc(-c6ccc7ccccc7c6)ccc45)nc(-c4cccc5oc6ccccc6c45)n3)cc2)cc1 ; "M-423"] | 68 |
| 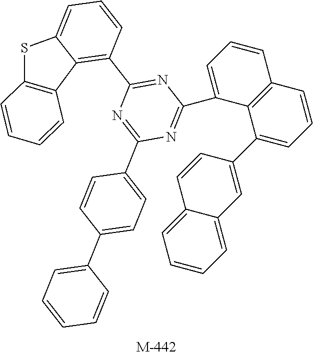 [SMILES: c1ccc(-c2ccc(-c3nc(-c4cccc5cccc(-c6ccc7ccccc7c6)c45)nc(-c4cccc5sc6ccccc6c45)n3)cc2)cc1 ; "M-442"] | 72 |
| 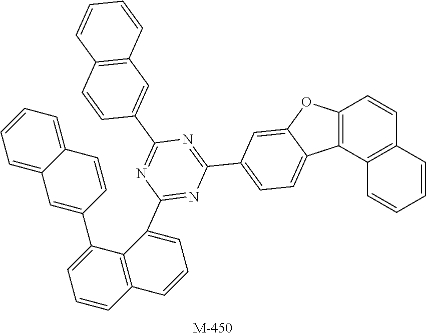 [SMILES: c1ccc2cc(-c3nc(-c4ccc5c(c4)oc4ccc6ccccc6c45)nc(-c4cccc5cccc(-c6ccc7ccccc7c6)c45)n3)ccc2c1 ; "M-450"] | 71 |
| 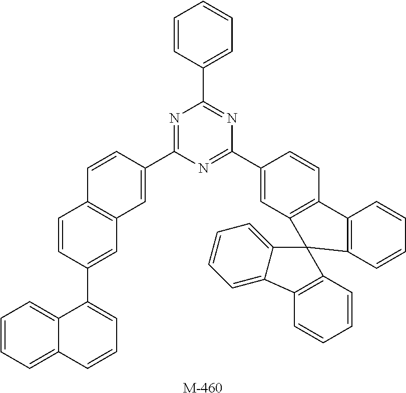 [SMILES: c1ccc(-c2nc(-c3ccc4c(c3)C3(c5ccccc5-c5ccccc53)c3ccccc3-4)nc(-c3ccc4ccc(-c5cccc6ccccc56)cc4c3)n2)cc1 ; "M-460"] | 68 |
| 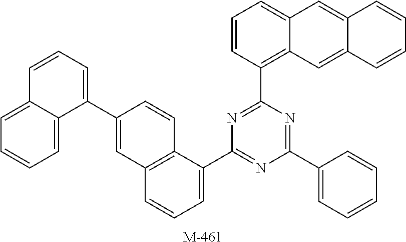 | 65 |
| 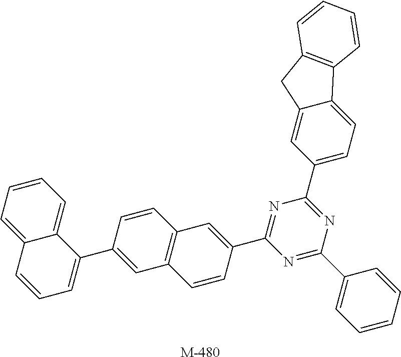 [SMILES: c1ccc(-c2nc(-c3ccc4c(c3)Cc3ccccc3-4)nc(-c3ccc4cc(-c5cccc6ccccc56)ccc4c3)n2)cc1 ; "M-480"] | 72 |
| 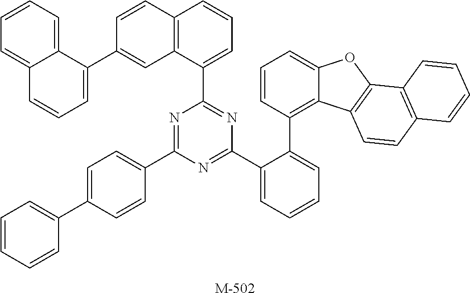 [SMILES: c1ccc(-c2ccc(-c3nc(-c4ccccc4-c4cccc5oc6c7ccccc7ccc6c45)nc(-c4cccc5ccc(-c6cccc7ccccc67)cc45)n3)cc2)cc1 ; "M-502"] | 68 |
| 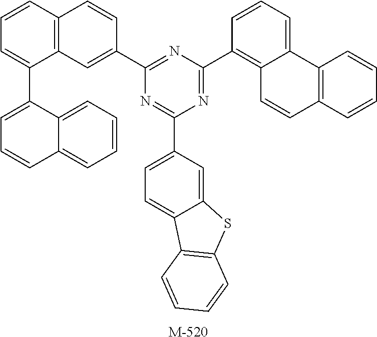 | 75 |
| 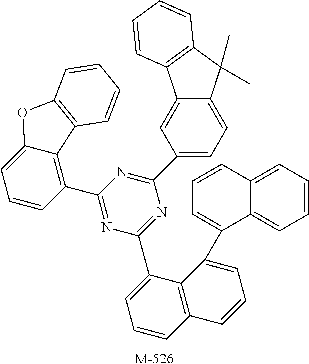 | 79 |
| 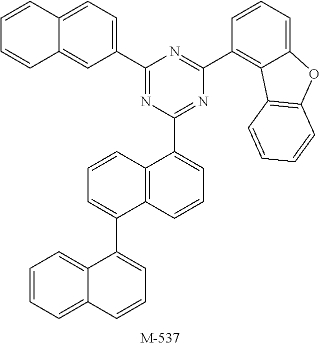 | 74 |
| 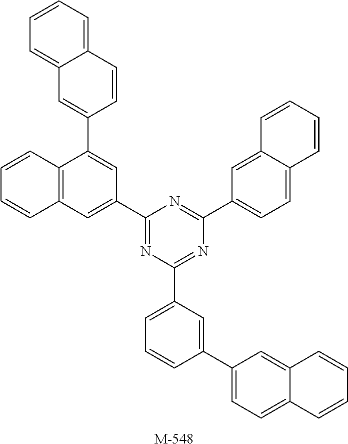 [SMILES: c1cc(-c2ccc3ccccc3c2)cc(-c2nc(-c3ccc4ccccc4c3)nc(-c3cc(-c4ccc5ccccc5c4)c4ccccc4c3)n2)c1 ; "M-548"] | 63 |
| 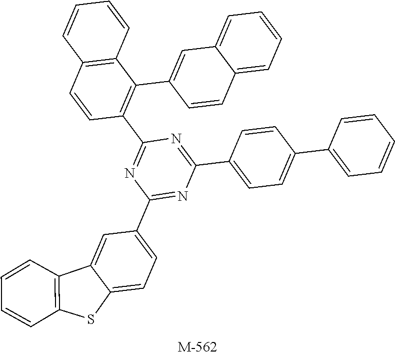 [SMILES: c1ccc(-c2ccc(-c3nc(-c4ccc5sc6ccccc6c5c4)nc(-c4ccc5ccccc5c4-c4ccc5ccccc5c4)n3)cc2)cc1 ; "M-562"] | 77 |
| 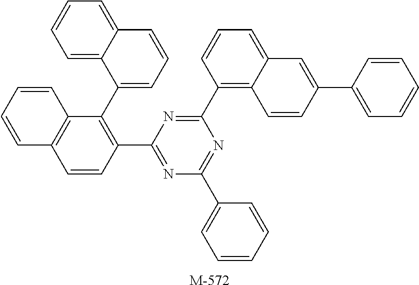 | 70 |
| 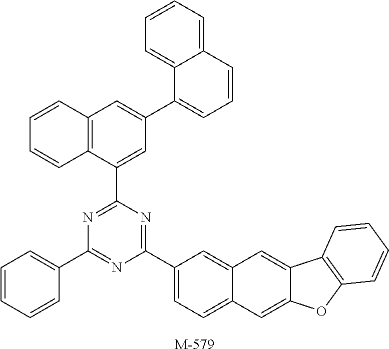 | 75 |
| 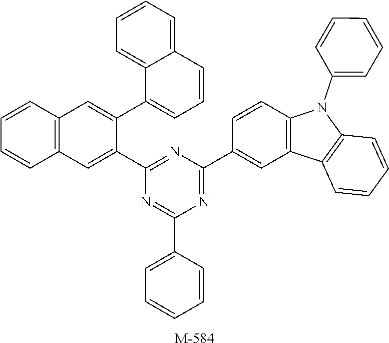 | 65 |
| 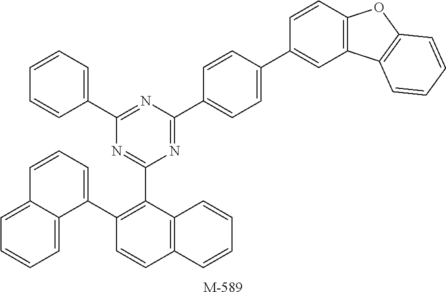 | 76 |
| 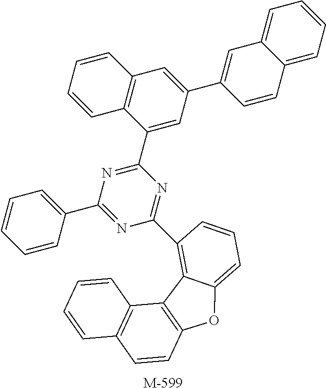 | 78 |
| 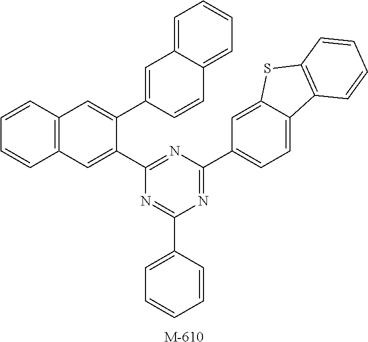 | 71 |
| 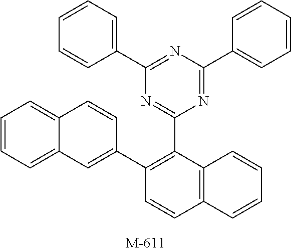 | 82 |
| 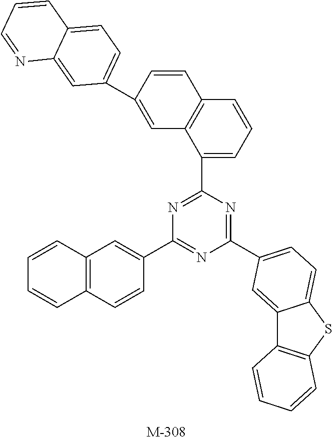 [SMILES: c1ccc2cc(-c3nc(-c4ccc5sc6ccccc6c5c4)nc(-c4cccc5ccc(-c6ccc7cccnc7c6)cc45)n3)ccc2c1 ; "M-308"] | 88 |
| 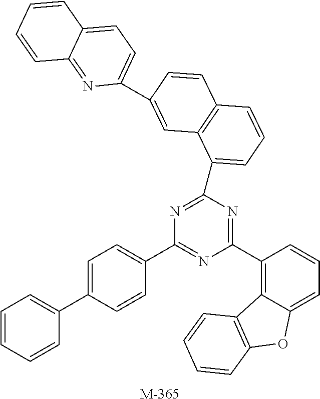 [SMILES: c1ccc(-c2ccc(-c3nc(-c4cccc5ccc(-c6ccc7ccccc7n6)cc45)nc(-c4cccc5oc6ccccc6c45)n3)cc2)cc1 ; "M-365"] | 80 |
| 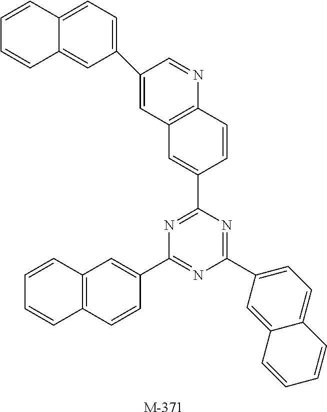 [SMILES: c1ccc2cc(-c3cnc4ccc(-c5nc(-c6ccc7ccccc7c6)nc(-c6ccc7ccccc7c6)n5)cc4c3)ccc2c1 ; "M-371"] | 80 |

TABLE 2

| | Elemental analysis | | HRMS (ESI) m/z [M + H]+ | |
|---|---|---|---|---|
| Compound | Theoretical | Measured | Theoretical | Measured |
| M-76 | C, 88.32; H, 4.57; N, 5.15; 0, 1.96; | C, 88.30; H, 4.57; N, 5.17; | 815.29 | 816.32 |
| M-108 | C, 85.54; H, 4.38; N, 7.30; O, 2.78; | C, 85.53; H, 4.37; N, 7.32; | 575.20 | 576.47 |
| M-145 | C, 86.57; H, 4.77; N, 8.65; | C, 86.56; H, 4.77; N, 8.67; | 485.19 | 486.33 |
| M-253 | C, 88.93; H, 4.72; N, 6.35; | C, 88.91; H, 4.73; N, 6.36; | 661.25 | 662.40 |
| M-394 | C, 86.38; H, 4.35; N, 6.72; O, 2.56 | C, 86.42; H, 4.34; N, 6.68; | 625.22 | 626.42 |
| M-412 | C, 88.35; H, 4.78; N, 6.87 | C, 88.39; H, 4.79; N, 6.82; | 611.24 | 612.38 |
| M-423 | C, 86.61; H, 4.49; N, 6.45; O, 2.45 | C, 86.64; H, 4.52; N, 6.39; | 651.23 | 656.37 |
| M-442 | C, 84.53; H, 4.38; N, 6.29; S, 4.80 | C, 84.56; H, 4.40; N, 6.23; | 667.21 | 668.64 |
| | | S, 4.81; | | |
| M-450 | C, 87.09; H, 4.33; N, 6.22; O, 2.37 | C, 87.12; H, 4.34; N, 6.19; | 675.23 | 676.19 |
| M-460 | C, 89.60; H, 4.60; N, 5.80 | C, 89.64; H, 4.61; N, 5.75; | 723.27 | 724.57 |
| M-461 | C, 88.18; H, 4.65; N, 7.17 | C, 88.22; H, 4.66; N, 7.12; | 585.22 | 586.39 |
| M-480 | C, 87.93; H, 4.74; N, 7.32 | C, 87.96; H, 4.77; N, 7.27; | 573.22 | 574.68 |
| M-502 | C, 88.01; H, 4.54; N, 5.40; O, 2.06 | C, 88.06; H, 4.55; N, 5.34; | 777.28 | 778.45 |
| M-520 | C, 85.07; H, 4.23; N, 6.07; S, 4.63 | C, 85.11; H, 4.24; N, 6.02; | 691.21 | 692.75 |
| | | S, 4.63; | | |
| M-526 | C, 86.81; H, 4.81; N, 6.07; O, 2.31 | C, 86.84; H, 4.83; N, 6.02; | 691.26 | 692.28 |
| M-537 | C, 86.38; H, 4.35; N, 6.72; O, 2.56 | C, 86.40; H, 4.36; N, 6.69; | 625.22 | 626.37 |
| M-548 | C, 88.93; H, 4.72; N, 6.35 | C, 88.96; H, 4.74; N, 6.30; | 661.25 | 662.48 |
| M-562 | C, 84.53; H, 4.38; N, 6.29; S, 4.80 | C, 84.57; H, 4.40; N, 6.22; | 667.21 | 668.44 |
| | | S, 4.81; | | |
| M-572 | C, 88.35; H, 4.78; N, 6.87 | C, 88.39; H, 4.79; N, 6.82; | 611.24 | 612.31 |
| M-579 | C, 86.38; H, 4.35; N, 6.72; O, 2.56 | C, 86.41; H, 4.36; N, 6.68; | 625.22 | 626.52 |
| M-584 | C, 86.74; H, 4.65; N, 8.61 | C, 86.77; H, 4.67; N, 8.56; | 650.25 | 651.38 |
| M-589 | C, 86.61; H, 4.49; N, 6.45; O, 2.45 | C, 86.64; H, 4.51; N, 6.40; | 651.23 | 652.18 |
| M-599 | C, 86.38; H, 4.35; N, 6.72; O, 2.56 | C, 86.40; H, 4.37; N, 6.67; | 625.22 | 626.47 |
| M-610 | C, 83.22; H, 4.26; N, 7.10; S, 5.42 | C, 83.25; H, 4.27; N, 7.04; | 591.18 | 592.04 |
| | | S, 5.44; | | |
| M-611 | C, 86.57; H, 4.77; N, 8.65 | C, 86.60; H, 4.79; N, 8.61; | 485.19 | 486.66 |
| M-308 | C, 82.22; H, 4.08; N, 8.72; S, 4.99; | C, 82.23; H, 4.07; N, 8.72; | 642.19 | 643.02 |
| | | S, 4.98; | | |
| M-365 | C, 84.64; H, 4.32; N, 8.58; O, 2.45; | C, 84.66; H, 4.32; N, 8.57; | 652.23 | 653.31 |
| M-371 | C, 85.98; H, 4.47; N, 9.55; | C, 85.99; H, 4.48; N, 9.53; | 586.22 | 587.77 |

### Example 5

An organic electroluminescent host material composition includes a compound N containing a polycyclic heteromatic group of triphenylene and a compound M, and the compound M is as shown in any one of M-1 to M-619, and is preferably selected from the organic electroluminescent compounds prepared in Examples 1-4; the compound N in the composition is a compound as shown in any one of N-1 to N-935, and is preferably any compound in N-4, N-5, N-14, N-20, N-37, N-46, N-65, N-68, N-78, N-113, N-275, N-281, N-358, N-369, N-389, N-423, N-424, N-434, N-439, N-447, N-470, N-486, N-505, N-517, N-520, N-529, N-533, N-629, N-641, N-669, N-685, N-728, N-860, N-874, N-899, N-901, N-906, N-912 and N-918; and synthesis processes of the above compounds N are as follows:

### 1. Synthetic Route of N-4

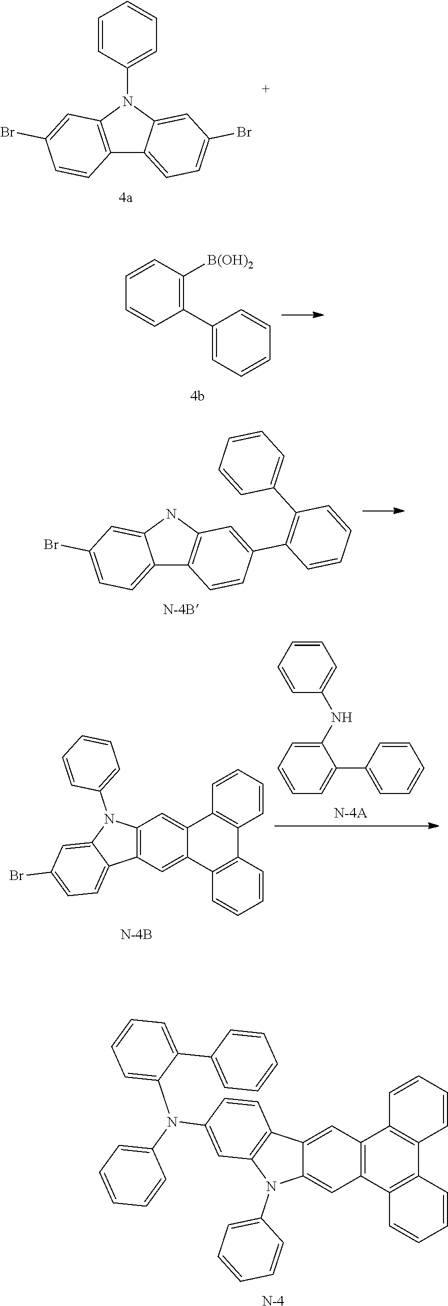

### 1.1 Synthesis of an Intermediate N-4B′

Under nitrogen purging, 20 g (1.0 eq) of 4a (CAS: 444796-09-2), 9.87 g (1.0 eq) of 4b (CAS: 4688-76-0), 1.7 g (2% eq) of Pd(PPh₃)₄, 8.37 g (2.0 eq) of NaHCO₃, 180 mL of tetrahydrofuran (4a:tetrahydrofuran=1 g:9 mL) and 60 mL of ultrapure water (4a:ultrapure water=1 g:3 mL) were added into a 500 mL three-necked flask with a magnetic stirrer, and completely reacted at 65° C. for 2 h. 10 g of a product N-4B′ was obtained through column purification.

### 1.2 Synthesis of an Intermediate N-4B

10 g (1.0 eq) of the intermediate N-4B′, 20.5 g (6.0 eq) of anhydrous FeCl₃, and 100 mL of anhydrous dichloromethane (N-4B′:dichloromethane=1 g:10 mL) were added into a 250 mL three-necked flask with a magnetic stirrer, and completely reacted at −10° C. for 1 h. 7 g of a product N-4B was obtained through column purification.

### 1.3 Synthesis of a Compound N-4

Under nitrogen purging, 7 g (1.0 eq) of N-4B, 4.0 g (1.1 eq) of N-4A (CAS: 35887-50-4), 0.27 g (2% eq) of Pd₂(dba)₃, 2.85 g (2.0 eq) of t-BuONa, and 70 mL of anhydrous toluene (N-4B:anhydrous toluene=1 g:10 mL) were added into a 250 mL three-necked flask with a magnetic stirrer, and completely reacted at 100° C. for 2 h. 5 g of a product N-4 was obtained through column purification.

Elemental analysis: C₄₈H₃₂N₂; theoretical value: C, 90.54; H, 5.07; N, 4.40; measured value: C, 90.53; H, 5.08; N, 4.41; HRMS (ESI) m/z (M+): theoretical value: 636.26; measured value: 637.55.

### 2. Synthetic Route of N-5

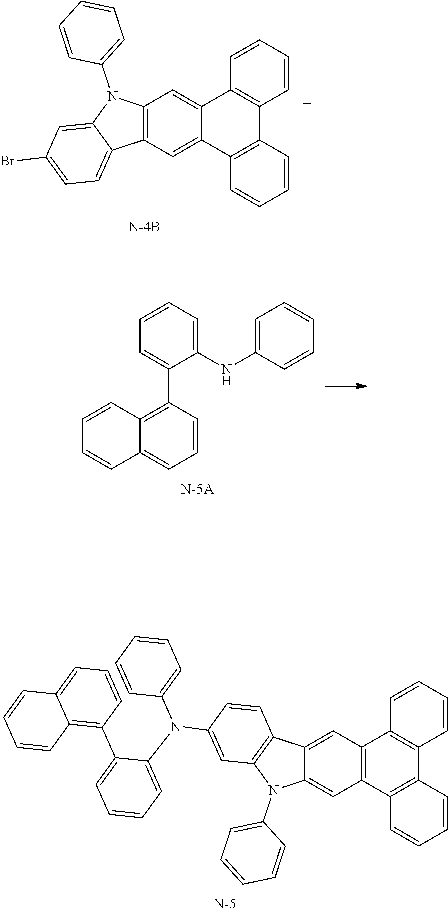 [SMILES: Brc1ccc2c3cc4c5ccccc5c5ccccc5c4cc3n(-c3ccccc3)c2c1 ; "N-4B" ; c1ccc(Nc2ccccc2-c2cccc3ccccc23)cc1 ; c1ccc(N(c2ccc3c4cc5c6ccccc6c6ccccc6c5cc4n(-c4ccccc4)c3c2)c2ccccc2-c2cccc3ccccc23)cc1]

Under nitrogen purging, 7 g (1.0 eq) of the intermediate N-4B, 4.82 g (1.1 eq) of N-5A (CAS: 1401351-42-5), 0.27 g (2% eq) of Pd₂(dba)₃, 2.85 g (2.0 eq) of t-BuONa, and 70 mL of anhydrous toluene (intermediate N-4B:anhydrous toluene=1 g:10 mL) were added into a 250 mL three-necked flask with a magnetic stirrer, and completely reacted at 100° C. for 2 h. 5 g of a product was obtained through column purification.

Elemental analysis: C₅₂H₃₄N₂; theoretical value: C, 90.93; H, 4.99; N, 4.08; measured value: C, 90.92; H, 4.98; N, 4.10; HRMS (ESI) m/z (M+): theoretical value: 686.27; measured value: 687.22.

### 3. Synthetic Route of N-14

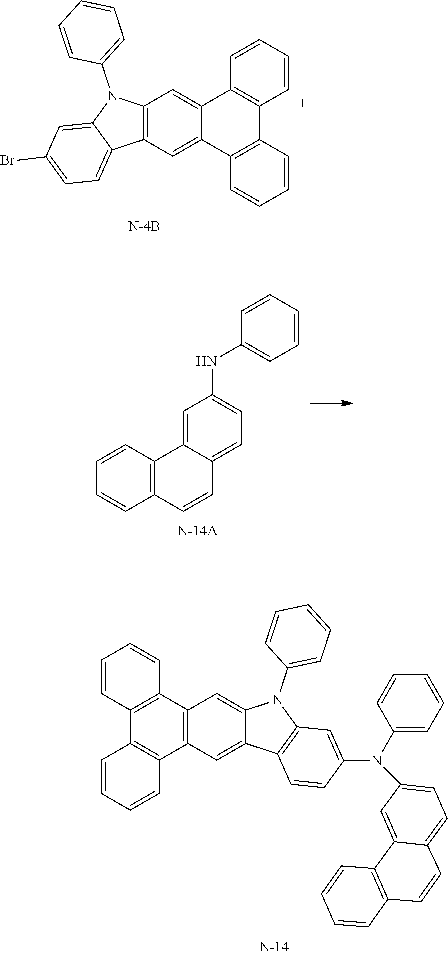

Under nitrogen purging, 7 g (1.0 eq) of the intermediate N-4B, 4.4 g (1.1 eq) of N-14A (CAS: 1357009-66-5), 0.27 g (2% eq) of Pd₂(dba)₃, 2.85 g (2.0 eq) of t-BuONa, and 70 mL of anhydrous toluene (intermediate 2:anhydrous toluene=1 g:10 mL) were added into a 250 mL three-necked flask with a magnetic stirrer, and completely reacted at 100° C. for 2 h. 5 g of a product was obtained through column purification.

Elemental analysis: C₅₀H₃₂N₂; theoretical value: C, 90.88; H, 4.88; N, 4.24; measured value: C, 90.86; H, 4.88; N, 4.26; HRMS (ESI) m/z (M+): theoretical value: 660.25; measured value: 661.37.

### 4. Synthetic Route of N-20, N-37, N-46, N-65, N-68, N-78, N-113, N-275, N-281, N-358, N-369 and N-389

Synthesis conditions were the same as those of N-14, and the difference was that the structures and yields of a raw material N-nA, a raw material N-nB and products were different, specifically as shown in Table 3 below; and the elemental analysis results of the prepared compounds were as shown in Table 4.

TABLE 3

| Raw material N-nA | Raw material N-nB |
|---|---|
| 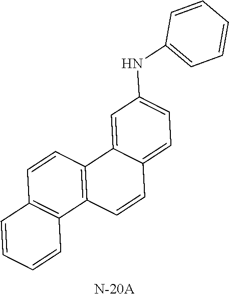 | 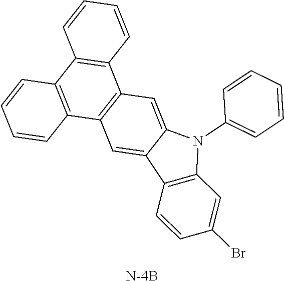 [SMILES: Brc1ccc2c3cc4c5ccccc5c5ccccc5c4cc3n(-c3ccccc3)c2c1 ; "N-4B"] |
| 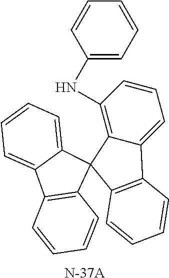 | 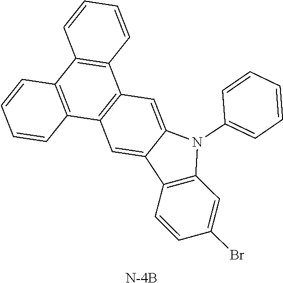 [SMILES: Brc1ccc2c3cc4c5ccccc5c5ccccc5c4cc3n(-c3ccccc3)c2c1 ; "N-4B"] |
| 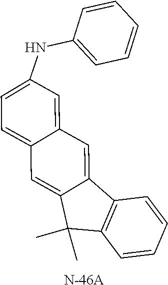 | 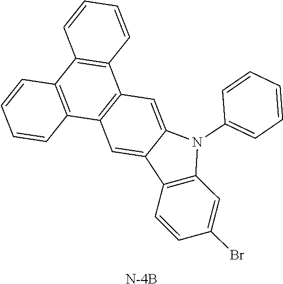 [SMILES: Brc1ccc2c3cc4c5ccccc5c5ccccc5c4cc3n(-c3ccccc3)c2c1 ; "N-4B"] |
| 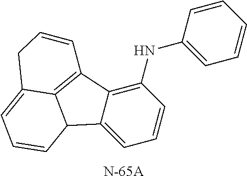 | 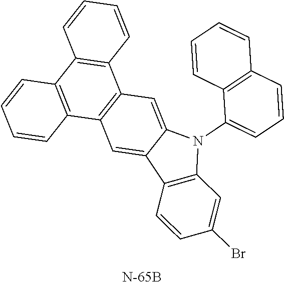 [SMILES: Brc1ccc2c3cc4c5ccccc5c5ccccc5c4cc3n(-c3cccc4ccccc34)c2c1 ; "N-65B"] |
| 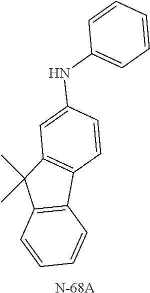 | 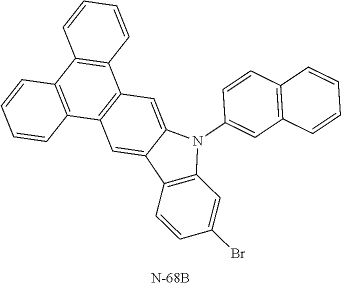 [SMILES: Brc1ccc2c3cc4c5ccccc5c5ccccc5c4cc3n(-c3ccc4ccccc4c3)c2c1 ; "N-68B"] |
| 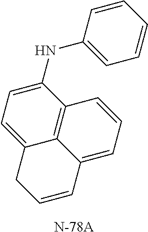 | 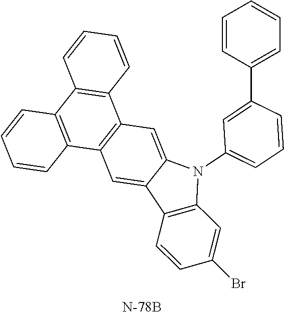 [SMILES: Brc1ccc2c3cc4c5ccccc5c5ccccc5c4cc3n(-c3cccc(-c4ccccc4)c3)c2c1 ; "N-78B"] |
| 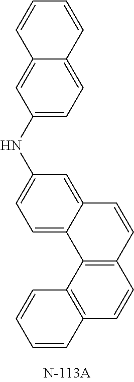 [SMILES: c1ccc2cc(Nc3ccc4c(ccc5ccc6ccccc6c54)c3)ccc2c1] | 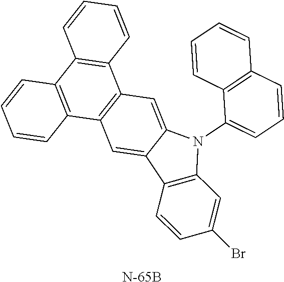 [SMILES: Brc1ccc2c3cc4c5ccccc5c5ccccc5c4cc3n(-c3cccc4ccccc34)c2c1 ; "N-65B"] |
| 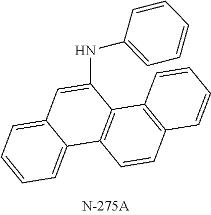 [SMILES: c1ccc(Nc2cc3ccccc3c3ccc4ccccc4c23)cc1] | 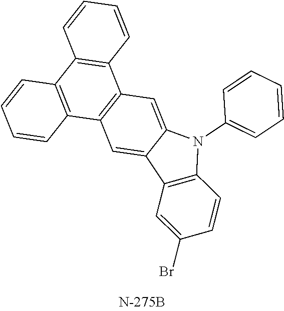 [SMILES: Brc1ccc2c(c1)c1cc3c4ccccc4c4ccccc4c3cc1n2-c1ccccc1] |
| 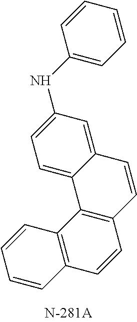 [SMILES: c1ccc(Nc2ccc3c(ccc4ccc5ccccc5c43)c2)cc1] | 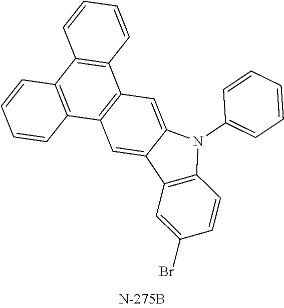 [SMILES: Brc1ccc2c(c1)c1cc3c4ccccc4c4ccccc4c3cc1n2-c1ccccc1] |
| 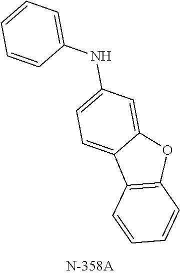 [SMILES: c1ccc(Nc2ccc3c(c2)oc2ccccc23)cc1] | 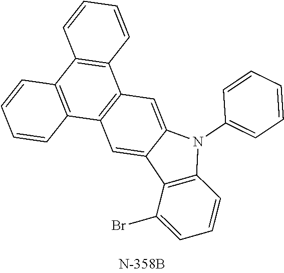 [SMILES: Brc1cccc2c1c1cc3c4ccccc4c4ccccc4c3cc1n2-c1ccccc1] |
| 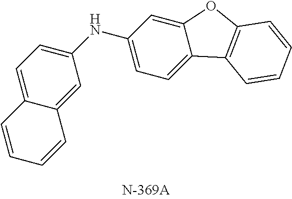 [SMILES: c1ccc2cc(Nc3ccc4c(c3)oc3ccccc34)ccc2c1] | 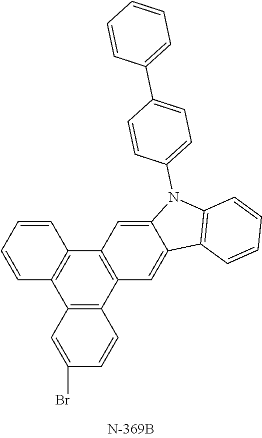 [SMILES: Brc1ccc2c(c1)c1ccccc1c1cc3c(cc21)c1ccccc1n3-c1ccc(-c2ccccc2)cc1] |
| 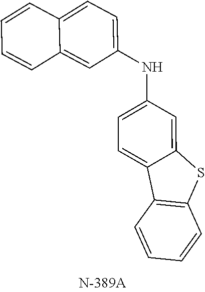 [SMILES: c1ccc2cc(Nc3ccc4c(c3)sc3ccccc34)ccc2c1] | 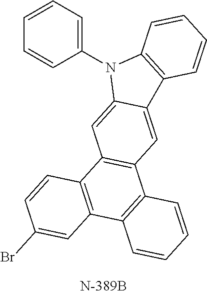 [SMILES: Brc1ccc2c(c1)c1ccccc1c1cc3c4ccccc4n(-c4ccccc4)c3cc21] |

| Products | Yield % |
|---|---|
| 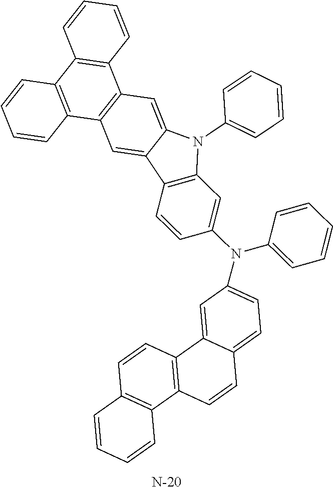 | 60 |
| 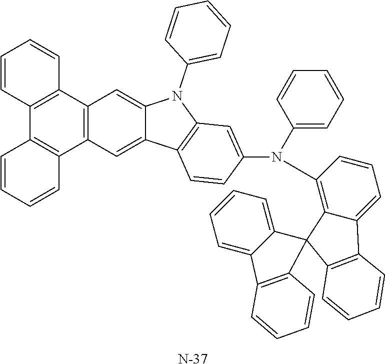 | 68 |
| 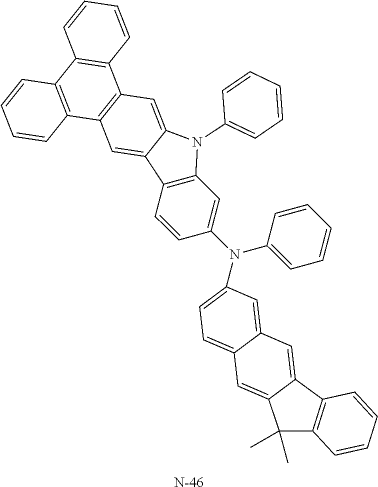 | 69 |
| 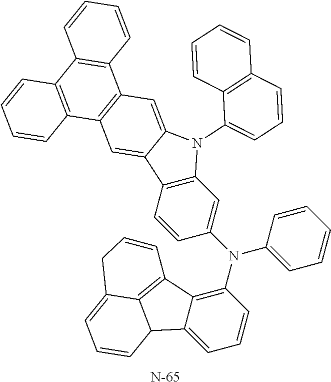 | 61 |
| 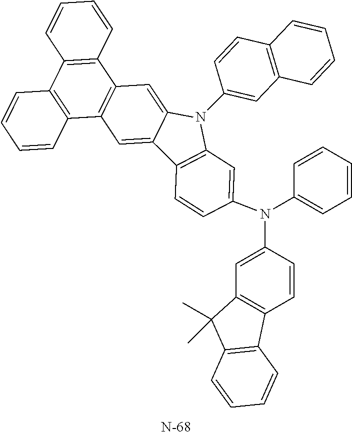 | 63 |
| 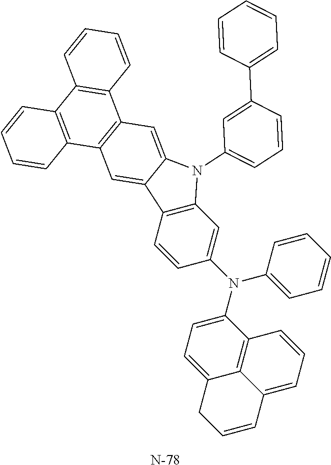 | 55 |
| 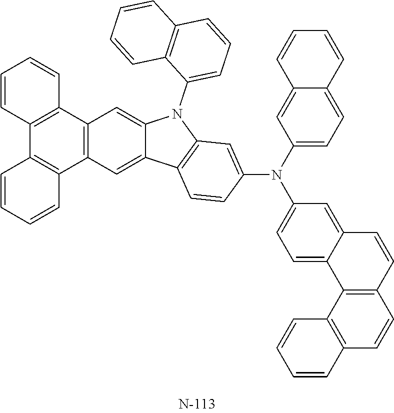 [SMILES: c1ccc2cc(N(c3ccc4c(ccc5ccc6ccccc6c54)c3)c3ccc4c5cc6c7ccccc7c7ccccc7c6cc5n(-c5cccc6ccccc56)c4c3)ccc2c1] | 67 |
| 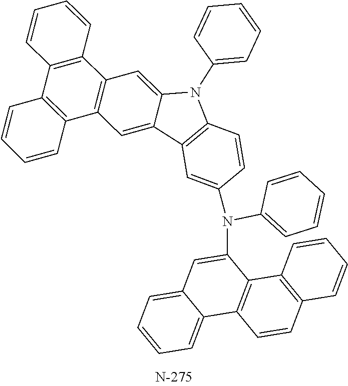 [SMILES: c1ccc(N(c2ccc3c(c2)c2cc4c5ccccc5c5ccccc5c4cc2n3-c2ccccc2)c2cc3ccccc3c3ccc4ccccc4c23)cc1] | 67 |
| 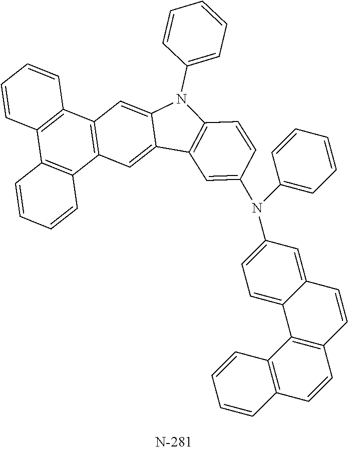 [SMILES: c1ccc(N(c2ccc3c(ccc4ccc5ccccc5c43)c2)c2ccc3c(c2)c2cc4c5ccccc5c5ccccc5c4cc2n3-c2ccccc2)cc1] | 63 |
| 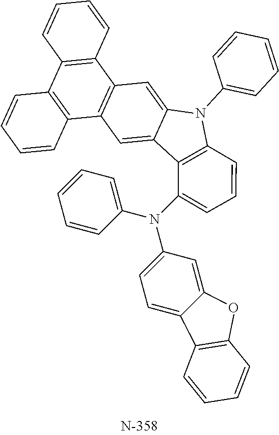 [SMILES: c1ccc(N(c2ccc3c(c2)oc2ccccc23)c2cccc3c2c2cc4c5ccccc5c5ccccc5c4cc2n3-c2ccccc2)cc1] | 72 |
| 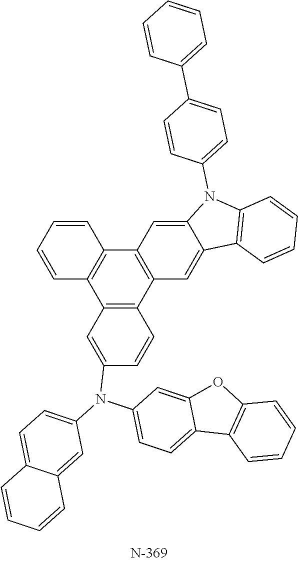 [SMILES: c1ccc(-c2ccc(-n3c4ccccc4c4cc5c6ccc(N(c7ccc8ccccc8c7)c7ccc8c(c7)oc7ccccc78)cc6c6ccccc6c5cc43)cc2)cc1] | 70 |
| 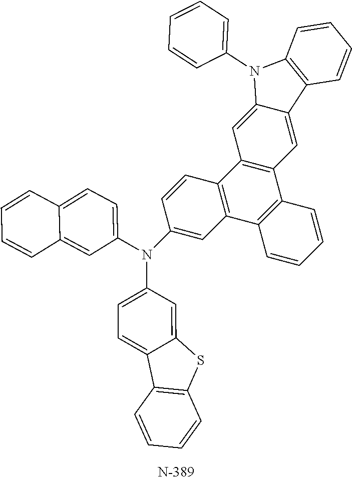 [SMILES: c1ccc(-n2c3ccccc3c3cc4c5ccccc5c5cc(N(c6ccc7ccccc7c6)c6ccc7c(c6)sc6ccccc67)ccc5c4cc32)cc1] | 72 |

TABLE 4

| | Elemental analysis | | HRMS (ESI) m/z [M + H]+ | |
|---|---|---|---|---|
| Compounds | Theoretical | Measured | Theoretical | Measured |
| N-20 | C, 91.24; H, 4.82; N, 3.94 | C, 91.22; H, 4.83; N, 3.95 | 710.27 | 711.37 |
| N-37 | C, 91.70; H, 4.79; N, 3.51 | C, 91.69; H, 4.79; N, 3.52 | 798.30 | 799.44 |
| N-46 | C, 90.88; H, 5.27; N, 3.85 | C, 90.87; H, 5.26; N, 3.87 | 726.30 | 727.41 |
| N-65 | C, 91.27; H, 4.92; N, 3.80 | C, 91.27; H, 4.92; N, 3.81 | 736.29 | 737.36 |
| N-68 | C, 90.88; H, 5.27; N, 3.85 | C, 90.87; H, 5.26; N, 3.87 | 726.30 | 727.51 |
| N-78 | C, 91.13; H, 5.01; N, 3.86 | C, 91.11; H, 5.00; N, 3.89 | 724.29 | 725.36 |
| N-113 | C, 91.82; H, 4.72; N, 3.45 | C, 91.81; H, 4.72; N, 3.47 | 810.30 | 811.42 |
| N-275 | C, 91.24; H, 4.82; N, 3.94 | C, 91.22; H, 4.81; N, 3.97 | 710.27 | 711.38 |
| N-281 | C, 91.24; H, 4.82; N, 3.94 | C, 91.23; H, 4.81; N, 3.96 | 710.27 | 711.45 |
| N-358 | C, 88.59; H, 4.65; N, 4.30; O, | C, 88.57; H, 4.66; N, 4.31; | 650.24 | 651.44 |
| | 2.46; | | | |
| N-369 | C, 89.66; H, 4.67; N, 3.61; O, | C, 89.64; H, 4.66; N, 3.64; | 776.29 | 777.11 |
| | 2.06; | | | |
| N-389 | C, 87.12; H, 4.50; N, 3.91; S, | C, 87.10; H, 4.50; N, 3.93; | 716.23 | 717.14 |
| | 4.47; | S, 4.47; | | |

### 5. Synthetic Route of N-423

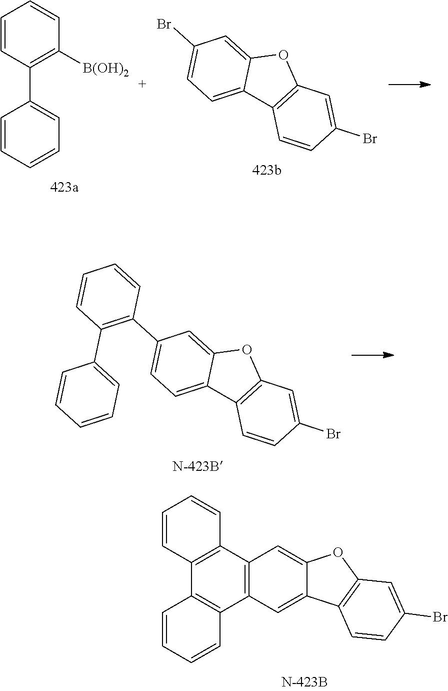 [SMILES: OB(O)c1ccccc1-c1ccccc1 ; Brc1ccc2c(c1)oc1cc(Br)ccc12 ; Brc1ccc2c(c1)oc1cc(-c3ccccc3-c3ccccc3)ccc12 ; Brc1ccc2c(c1)oc1cc3c4ccccc4c4ccccc4c3cc12]

20 g of 423a (i.e., 4b), 29.9 g of 423b (CAS: 67019-91-4), 2.3 g of Pd[P(C₆H₅)₃]₄, 27.9 g of K₂CO₃, 280 mL of toluene, 120 mL of H₂O and 120 mL of ethanol were added into a 1000 mL three-necked flask with a mechanical stirrer, a reflux condenser tube and a thermometer. Nitrogen replacement was conducted three times, and a reaction was conducted at 85° C. under a nitrogen protection condition. A reaction was started from temperature rise for 150 min. After the reaction was finished, 120 mL of water was added for a quenching reaction, after liquid separation, an oily substance (wet weight of 44 g) was obtained through spin-drying, and 25 g of N-423B′ was obtained after vacuumizing.

25 g of an N-423B′ crude product (oily substance) and 750 mL of dichloromethane (N-423B′:DCM=1 g:30 mL) were added into a 2 L three-necked flask with a stirrer and a thermometer, the temperature was controlled at −5° C., ferric trichloride was added in two batches, 3 equivalents of ferric trichloride were added every 15 min each batch, and the temperature was controlled at −5° C. After the reaction was finished, 750 mL of ethanol (N-423B′:ethanol=1 g:30 mL) was added slowly, and the temperature was controlled at 0° C. or below. After the ethanol was added, continuous stirring was conducted for 0.5 h, with a yellowish-white solid precipitated. Filtering was conducted, and a filter cake was leached with 250 mL of ethanol (N-423B′:ethanol=1 g:1 mL) to obtain a yellow solid. Then, desolvation and crystallization were conducted after 1.75 L of chlorobenzene was used for desolvation and crystallization after dissolution to clarification, when the temperature drops to 60° C. naturally, 250 mL of an n-hexane solution was added dropwise, and filtering was conducted to obtain 20 g of a crude product N-423B.

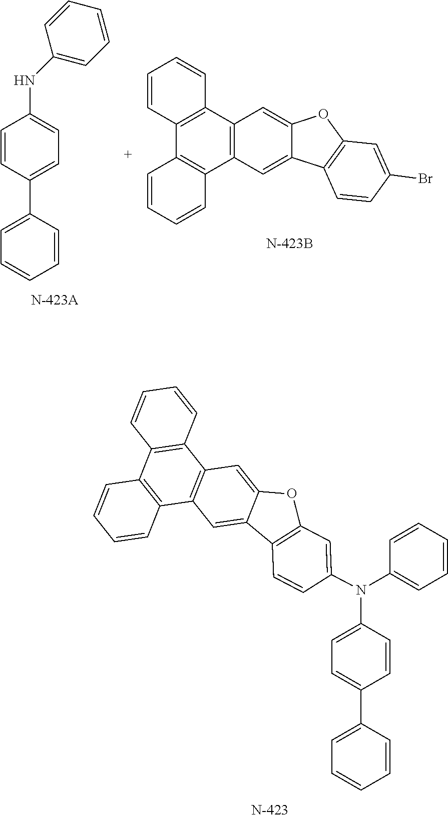 [SMILES: c1ccc(Nc2ccc(-c3ccccc3)cc2)cc1 ; Brc1ccc2c(c1)oc1cc3c4ccccc4c4ccccc4c3cc12 ; c1ccc(-c2ccc(N(c3ccccc3)c3ccc4c(c3)oc3cc5c6ccccc6c6ccccc6c5cc34)cc2)cc1]

25 g of N-423A (CAS: 32228-99-2), 40.48 g of N-423B (CAS: 2035812-74-7), 1.86 g of Pd₂(dba)₃, 1.67 g of sphos, 24.4 g of t-BuONa, and 500 mL of toluene were added into a 1000 mL three-necked flask with a mechanical stirrer, a reflux condenser tube and a thermometer. Nitrogen replacement was conducted three times, and a reaction was conducted at 110° C. under a nitrogen protection condition. A reaction was started from temperature rise for 120 min. After the reaction was finished, 120 mL of water was added for a quenching reaction, after liquid separation, spin-drying was conducted, and after column chromatography, 35 g of a crude product N-423 was obtained after drying.

Elemental analysis: C₄₂H₂₇NO; theoretical value: C, 89.81; H, 4.85; N, 2.49; O, 2.85; measured value: C, 89.78; H, 4.86; N, 2.51; HRMS (ESI) m/z (M+): theoretical value: 561.21; measured value: 562.29.

### 6. Synthetic Route of N-425

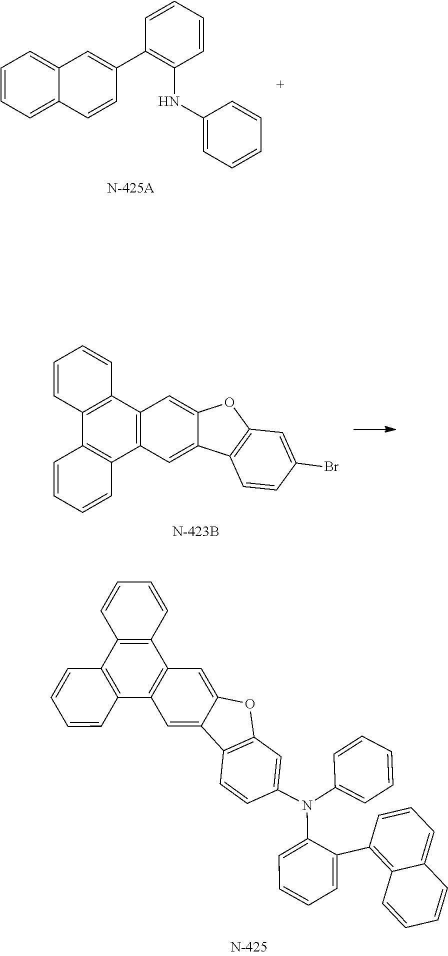 [SMILES: c1ccc(Nc2ccccc2-c2ccc3ccccc3c2)cc1 ; Brc1ccc2c(c1)oc1cc3c4ccccc4c4ccccc4c3cc12 ; c1ccc(N(c2ccc3c(c2)oc2cc4c5ccccc5c5ccccc5c4cc23)c2ccccc2-c2cccc3ccccc23)cc1]

14 g of N-425A (CAS: 1401351-43-6), 18.8 g of N-423B (CAS: 2035812-74-7), 0.86 g of Pd₂(dba)₃, 1.9 g of sphos, 9.1 g of t-BuONa, and 150 mL of toluene were added into a 500 mL three-necked flask with a mechanical stirrer, a reflux condenser tube and a thermometer. Nitrogen replacement was conducted three times, and a reaction was conducted at 110° C. under a nitrogen protection condition. A reaction was started from temperature rise for 120 min. After the reaction was finished, 150 mL of water was added for a quenching reaction, after liquid separation, spin-drying was conducted, and after beating with ethanol was conducted, desolvation and crystallization were conducted with toluene to obtain 16 g of a crude product N-425.

Elemental analysis: C₄₆H₂₉NO; theoretical value: C, 90.32; H, 4.78; N, 2.29; O, 2.62; measured value: C, 90.30; H, 4.77; N, 2.32; HRMS (ESI) m/z (M+): theoretical value: 611.22; measured value: 612.45.

### 7. Synthetic Route of N-424, N-434, N-439, N-447, N-470, N-486, N-505, N-517, N-520, N-529, N-533, N-629, N-641, N-669, N-685, N-728, N-860 and N-874

Synthesis conditions were the same as those of N-425, and the difference was that the structures and yields of a raw material N-nA, a raw material N-nB and products were different, specifically as shown in Table 5 below; and the elemental analysis results of the prepared compounds were as shown in Table 6.

TABLE 5

| Raw material N-nA | Raw material N-nB |
|---|---|
| 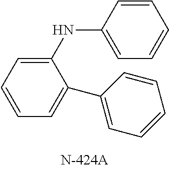 [SMILES: c1ccc(Nc2ccccc2-c2ccccc2)cc1] | 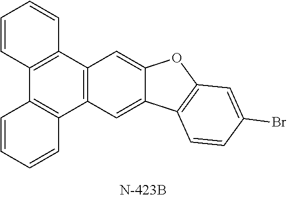 [SMILES: Brc1ccc2c(c1)oc1cc3c4ccccc4c4ccccc4c3cc12] |
| 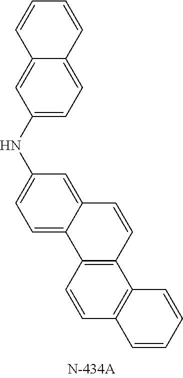 [SMILES: c1ccc2cc(Nc3ccc4c(ccc5c6ccccc6ccc45)c3)ccc2c1] | 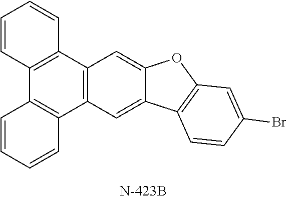 [SMILES: Brc1ccc2c(c1)oc1cc3c4ccccc4c4ccccc4c3cc12] |
| 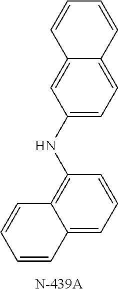 [SMILES: c1ccc2cc(Nc3cccc4ccccc34)ccc2c1] | 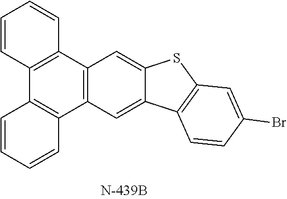 [SMILES: Brc1ccc2c(c1)sc1cc3c4ccccc4c4ccccc4c3cc12] |
| 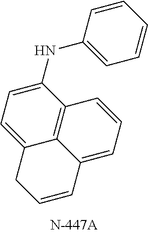 [SMILES: C1=Cc2cccc3c(Nc4ccccc4)ccc(c23)C1] | 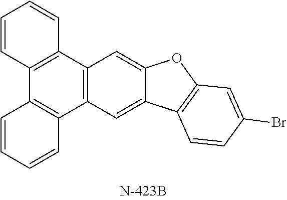 [SMILES: Brc1ccc2c(c1)oc1cc3c4ccccc4c4ccccc4c3cc12] |
| 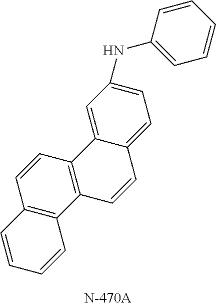 [SMILES: c1ccc(Nc2ccc3ccc4c5ccccc5ccc4c3c2)cc1] | 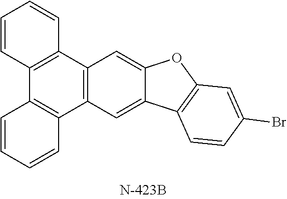 [SMILES: Brc1ccc2c(c1)oc1cc3c4ccccc4c4ccccc4c3cc12] |
| 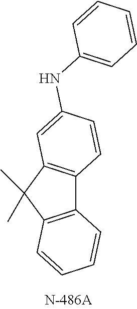 [SMILES: CC1(C)c2ccccc2-c2ccc(Nc3ccccc3)cc21] | 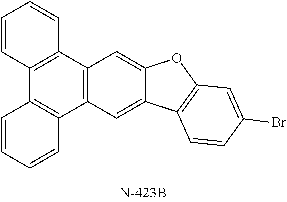 [SMILES: Brc1ccc2c(c1)oc1cc3c4ccccc4c4ccccc4c3cc12] |
| 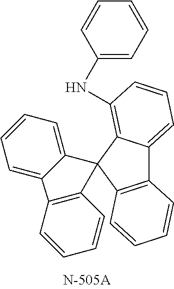 [SMILES: c1ccc(Nc2cccc3c2C2(c4ccccc4-c4ccccc42)c2ccccc2-3)cc1] | 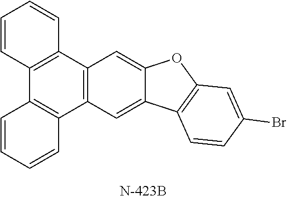 [SMILES: Brc1ccc2c(c1)oc1cc3c4ccccc4c4ccccc4c3cc12] |
| 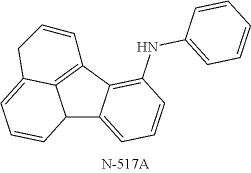 [SMILES: C1=CC2C3=C(C=CCC3=C1)c1c(Nc3ccccc3)cccc12] | 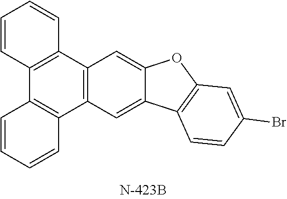 [SMILES: Brc1ccc2c(c1)oc1cc3c4ccccc4c4ccccc4c3cc12] |
| 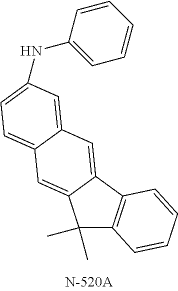 [SMILES: CC1(C)c2ccccc2-c2cc3cc(Nc4ccccc4)ccc3cc21] | 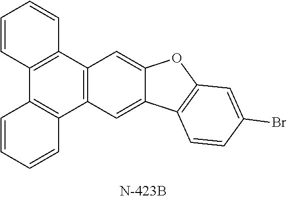 [SMILES: Brc1ccc2c(c1)oc1cc3c4ccccc4c4ccccc4c3cc12] |
| 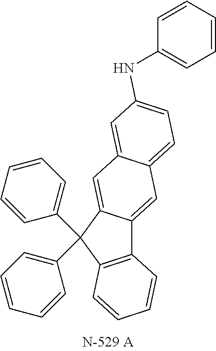 [SMILES: c1ccc(Nc2ccc3cc4c(cc3c2)C(c2ccccc2)(c2ccccc2)c2ccccc2-4)cc1] | 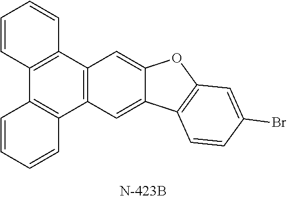 [SMILES: Brc1ccc2c(c1)oc1cc3c4ccccc4c4ccccc4c3cc12] |
| 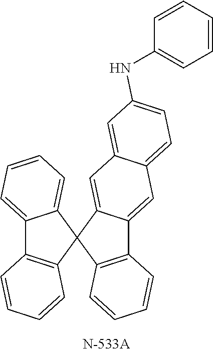 [SMILES: c1ccc(Nc2ccc3cc4c(cc3c2)C2(c3ccccc3-c3ccccc32)c2ccccc2-4)cc1] | 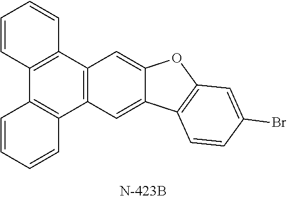 [SMILES: Brc1ccc2c(c1)oc1cc3c4ccccc4c4ccccc4c3cc12] |
| 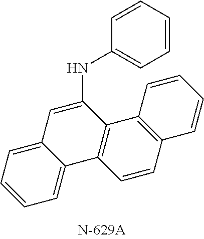 [SMILES: c1ccc(Nc2cc3ccccc3c3ccc4ccccc4c23)cc1] | 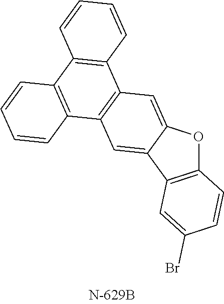 [SMILES: Brc1ccc2oc3cc4c5ccccc5c5ccccc5c4cc3c2c1] |
| 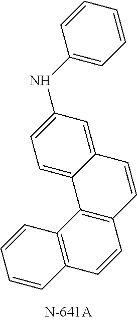 [SMILES: c1ccc(Nc2ccc3c(ccc4ccc5ccccc5c43)c2)cc1] | 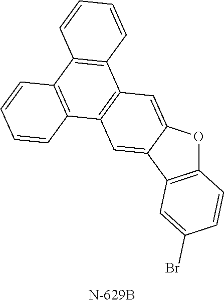 [SMILES: Brc1ccc2oc3cc4c5ccccc5c5ccccc5c4cc3c2c1] |
| 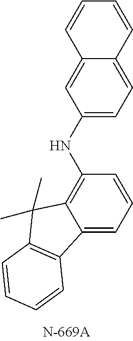 [SMILES: CC1(C)c2ccccc2-c2cccc(Nc3ccc4ccccc4c3)c21] | 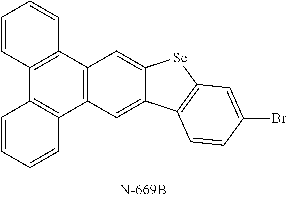 [SMILES: Brc1ccc2c(c1)[se]c1cc3c4ccccc4c4ccccc4c3cc12] |
| 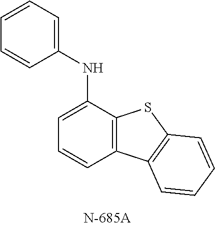 [SMILES: c1ccc(Nc2cccc3c2sc2ccccc23)cc1] | 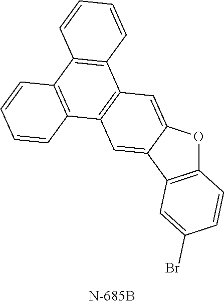 [SMILES: Brc1ccc2oc3cc4c5ccccc5c5ccccc5c4cc3c2c1] |
| 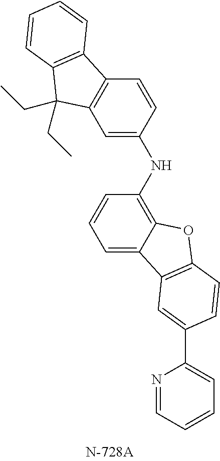 [SMILES: CCC1(CC)c2ccccc2-c2ccc(Nc3cccc4c3oc3ccc(-c5ccccn5)cc34)cc21] | 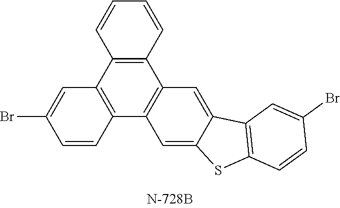 [SMILES: Brc1ccc2sc3cc4c5ccc(Br)cc5c5ccccc5c4cc3c2c1] |
| 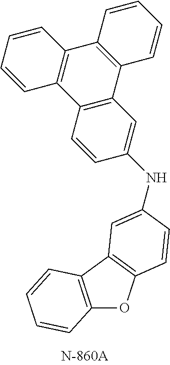 [SMILES: c1ccc2c(c1)oc1ccc(Nc3ccc4c5ccccc5c5ccccc5c4c3)cc12] | 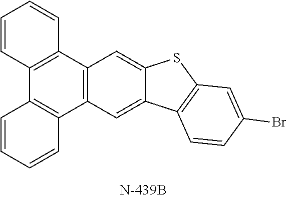 [SMILES: Brc1ccc2c(c1)sc1cc3c4ccccc4c4ccccc4c3cc12] |
| 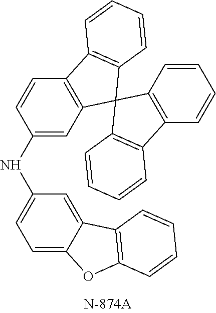 [SMILES: c1ccc2c(c1)-c1ccccc1C21c2ccccc2-c2ccc(Nc3ccc4oc5ccccc5c4c3)cc21] | 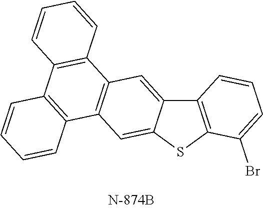 [SMILES: Brc1cccc2c1sc1cc3c4ccccc4c4ccccc4c3cc12] |

| Products | Yield % |
|---|---|
| 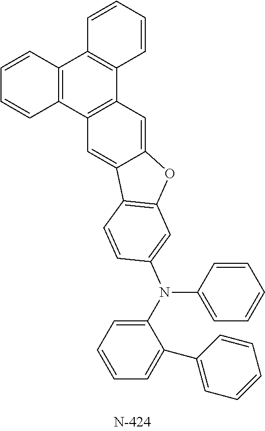 [SMILES: c1ccc(-c2ccccc2N(c2ccccc2)c2ccc3c(c2)oc2cc4c5ccccc5c5ccccc5c4cc23)cc1] | 60 |
| 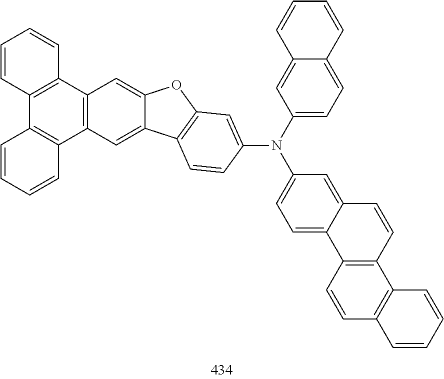 [SMILES: c1ccc2cc(N(c3ccc4c(ccc5c6ccccc6ccc45)c3)c3ccc4c(c3)oc3cc5c6ccccc6c6ccccc6c5cc34)ccc2c1] | 65 |
| 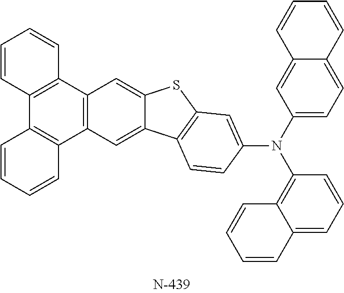 [SMILES: c1ccc2cc(N(c3ccc4c(c3)sc3cc5c6ccccc6c6ccccc6c5cc34)c3cccc4ccccc34)ccc2c1] | 67 |
| 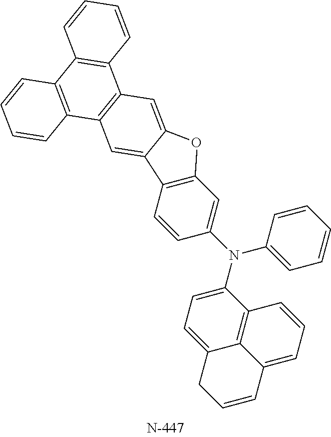 [SMILES: C1=Cc2cccc3c(N(c4ccccc4)c4ccc5c(c4)oc4cc6c7ccccc7c7ccccc7c6cc45)ccc(c23)C1] | 66 |
| 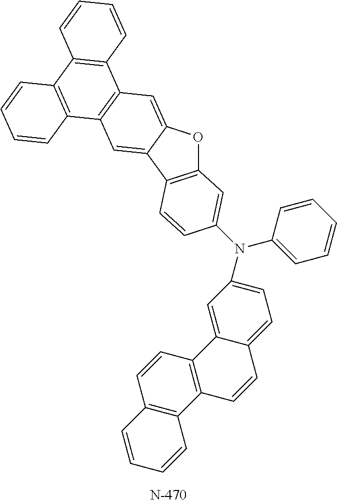 [SMILES: c1ccc(N(c2ccc3c(c2)oc2cc4c5ccccc5c5ccccc5c4cc23)c2ccc3ccc4c5ccccc5ccc4c3c2)cc1] | 62 |
| 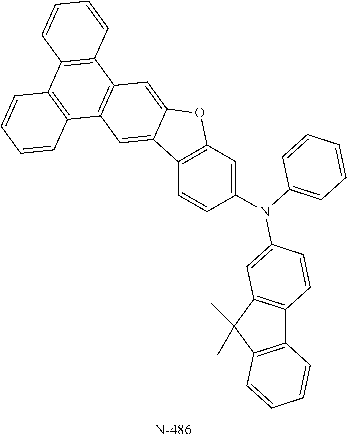 [SMILES: CC1(C)c2ccccc2-c2ccc(N(c3ccccc3)c3ccc4c(c3)oc3cc5c6ccccc6c6ccccc6c5cc34)cc21] | 59 |
| 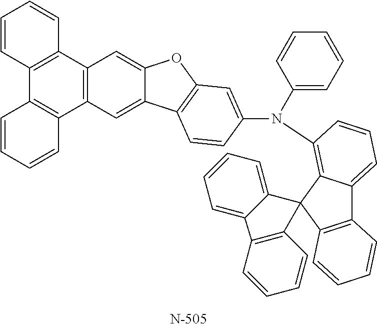 [SMILES: c1ccc(N(c2ccc3c(c2)oc2cc4c5ccccc5c5ccccc5c4cc23)c2cccc3c2C2(c4ccccc4-c4ccccc42)c2ccccc2-3)cc1] | 57 |
| 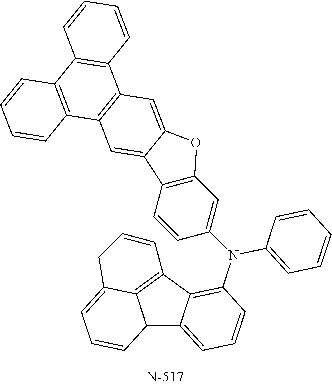 [SMILES: C1=CC2C3=C(C=CCC3=C1)c1c2cccc1N(c1ccccc1)c1ccc2c(c1)oc1cc3c4ccccc4c4ccccc4c3cc12] | 63 |
| 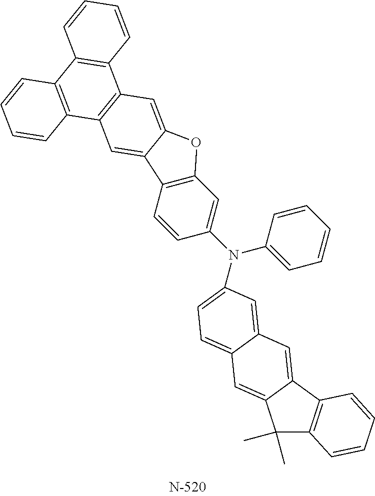 [SMILES: CC1(C)c2ccccc2-c2cc3cc(N(c4ccccc4)c4ccc5c(c4)oc4cc6c7ccccc7c7ccccc7c6cc45)ccc3cc21] | 70 |
| 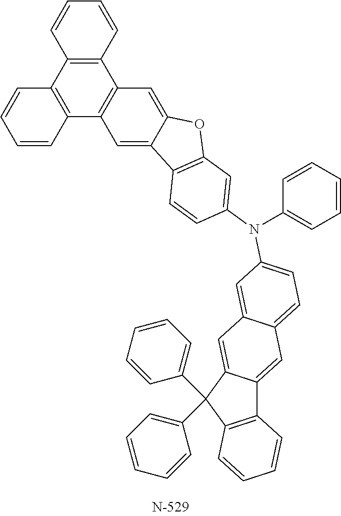 [SMILES: c1ccc(N(c2ccc3cc4c(cc3c2)C(c2ccccc2)(c2ccccc2)c2ccccc2-4)c2ccc3c(c2)oc2cc4c5ccccc5c5ccccc5c4cc23)cc1] | 73 |
| 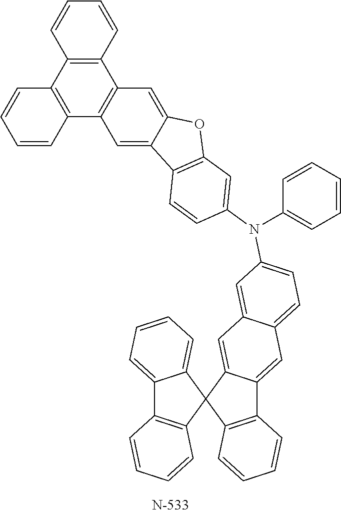 [SMILES: c1ccc(N(c2ccc3cc4c(cc3c2)C2(c3ccccc3-c3ccccc32)c2ccccc2-4)c2ccc3c(c2)oc2cc4c5ccccc5c5ccccc5c4cc23)cc1] | 69 |
| 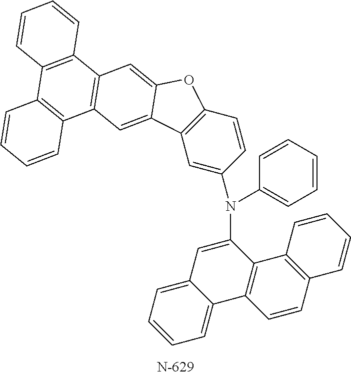 [SMILES: c1ccc(N(c2ccc3oc4cc5c6ccccc6c6ccccc6c5cc4c3c2)c2cc3ccccc3c3ccc4ccccc4c23)cc1] | 56 |
| 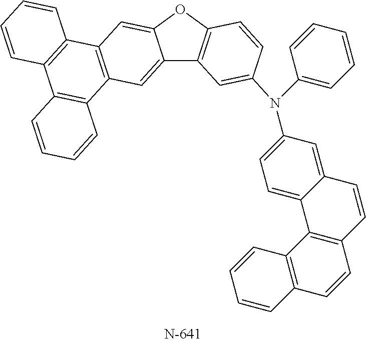 [SMILES: c1ccc(N(c2ccc3c(ccc4ccc5ccccc5c43)c2)c2ccc3oc4cc5c6ccccc6c6ccccc6c5cc4c3c2)cc1] | 55 |
| 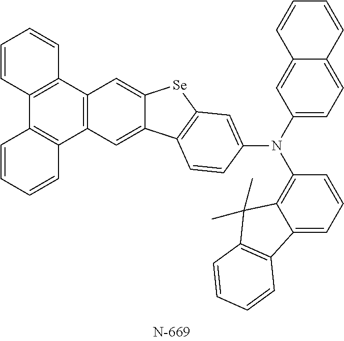 [SMILES: CC1(C)c2ccccc2-c2cccc(N(c3ccc4ccccc4c3)c3ccc4c(c3)[se]c3cc5c6ccccc6c6ccccc6c5cc34)c21] | 64 |
| 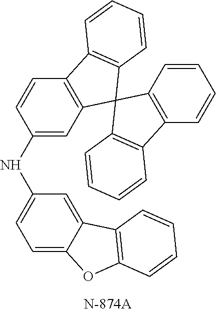 [SMILES: c1ccc2c(c1)-c1ccccc1C21c2ccccc2-c2ccc(Nc3ccc4oc5ccccc5c4c3)cc21] | 79 |
| 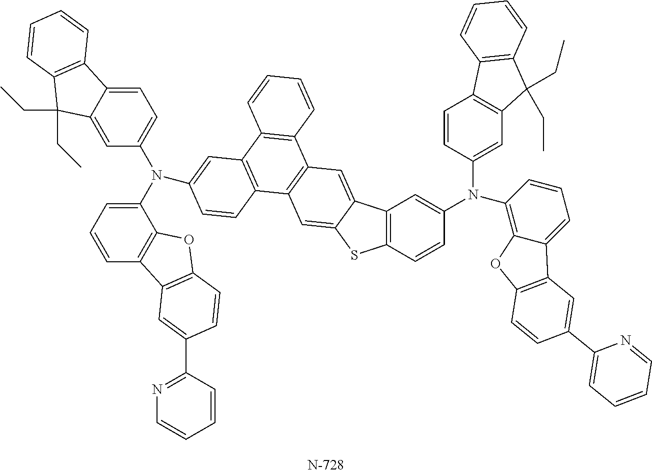 [SMILES: CCC1(CC)c2ccccc2-c2ccc(N(c3ccc4sc5cc6c7ccc(N(c8ccc9c(c8)C(CC)(CC)c8ccccc8-9)c8cccc9c8oc8ccc(-c%10ccccn%10)cc89)cc7c7ccccc7c6cc5c4c3)c3cccc4c3oc3ccc(-c5ccccn5)cc34)cc21] | 62 |
| 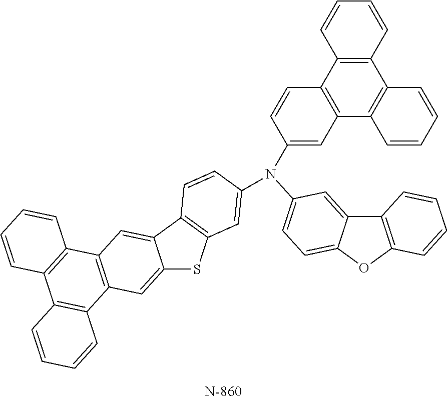 [SMILES: c1ccc2c(c1)oc1ccc(N(c3ccc4c(c3)sc3cc5c6ccccc6c6ccccc6c5cc34)c3ccc4c5ccccc5c5ccccc5c4c3)cc12] | 64 |
| 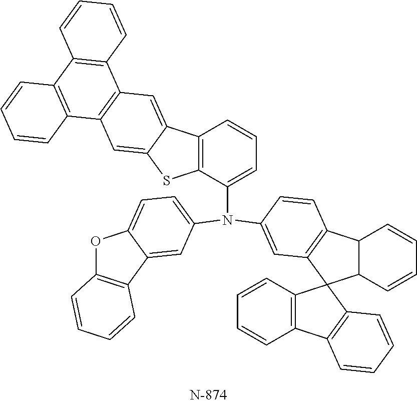 [SMILES: C1=CC2c3ccc(N(c4ccc5oc6ccccc6c5c4)c4cccc5c4sc4cc6c7ccccc7c7ccccc7c6cc45)cc3C3(c4ccccc4-c4ccccc43)C2C=C1] | 70 |

TABLE 6

| | Elemental analysis | | HRMS (ESI) m/z [M + H]+ | |
|---|---|---|---|---|
| Compounds | Theoretical | Measured | Theoretical | Measured |
| N-424 | C, 89.81; H, 4.85; N, 2.49; | C, 89.79; H, 4.86; N, 2.50; | 561.21 | 562.33 |
| | O, 2.85 | | | |
| N-434 | C, 91.07; H, 4.56; N, 2.04; | C, 91.04; H, 4.57; N, 2.06; | 685.24 | 686.23 |
| | O, 2.33 | | | |
| N-439 | C, 87.82; H, 4.52; N, 2.33; | C, 87.80; H, 4.52; N, 2.34; S, | 601.19 | 602.54 |
| | S, 5.33 | 5.34 | | |
| N-447 | C, 90.03; H, 4.74; N, 2.44; | C, 90.01; H, 4.75; N, 2.45; | 573.21 | 574.33 |
| | O, 2.79 | | | |
| N-470 | C, 90.68; H, 4.60; N, 2.20; | C, 90.66; H, 4.60; N, 2.22; | 635.22 | 636.64 |
| | O, 2.52 | | | |
| N-486 | C, 89.82; H, 5.19; N, 2.33; | C, 89.79; H, 5.20; N, 2.35; | 601.24 | 602.54 |
| | O, 2.66 | | | |
| N-505 | C, 91.26; H, 4.60; N, 1.94; | C, 91.24; H, 4.61; N, 1.95; | 723.26 | 724.65 |
| | O, 2.21 | | | |
| N-517 | C, 90.32; H, 4.78; N, 2.29; | C, 90.30; H, 4.78; N, 2.31; | 611.22 | 612.35 |
| | O, 2.62 | | | |
| N-520 | C, 90.29; H, 5.10; N, 2.15; | C, 90.28; H, 5.10; N, 2.16; | 651.26 | 652.36 |
| | O, 2.45 | | | |
| N-529 | C, 91.33; H, 4.81; N, 1.81; | C, 91.31; H, 4.82; N, 1.82; | 775.29 | 776.73 |
| | O,2.06 | | | |
| N-533 | C, 91.56; H, 4.56; N, 1.81; | C, 91.54; H, 4.55; N, 1.84; | 773.27 | 774.81 |
| | O, 2.07 | | | |
| N-629 | C, 90.68; H, 4.60; N, 2.20; | C, 90.66; H, 4.59; N, 2.22; | 635.22 | 636.29 |
| | O, 2.52 | | | |
| N-641 | C, 90.68; H, 4.60; N, 2.20; | C, 90.66; H, 4.60; N, 2.21; | 635.22 | 636.31 |
| | O, 2.52 | | | |
| N-669 | C, 82.34; H, 4.65; N, 1.96; | C, 82.31; H, 4.66; N, 1.98; | 715.18 | 716.32 |
| | Se, 11.05 | Se, 11.05 | | |
| N-685 | C, 85.25; H, 4.26; N, 2.37; | C, 85.23; H, 4.25; N, 2.39; S, | 591.16 | 592.21 |
| | O, 2.70; S, 5.42 | 5.42 | | |
| N-728 | C, 85.55; H, 5.15; N, 4.34; | C, 85.52; H, 5.16; N, 4.36; S, | 1290.49 | 1291.11 |
| | 0, 2.48; S, 2.48 | 2.48 | | |
| N-860 | C, 87.42; H, 4.21; N, 1.89; | C, 87.38; H, 4.22; N, 1.91; S, | 741.21 | 742.01 |
| | O, 2.16; S, 4.32 | 4.33 | | |
| N-874 | C, 88.06; H, 4.48; N, 1.68; | C, 88.04; H, 4.48; N, 1.70; S, | 831.26 | 832.09 |
| | O, 1.92; S, 3.85 | 3.86 | | |

### 8. Synthetic Route of N-912

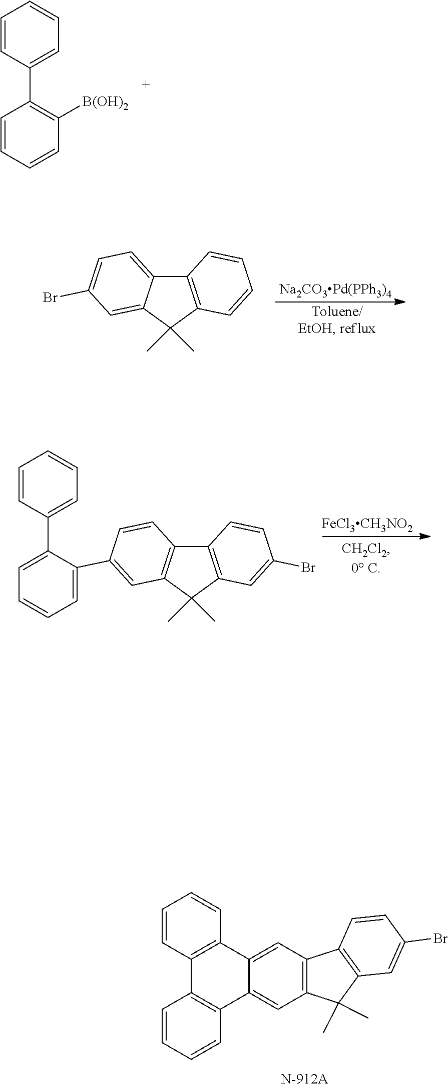 [SMILES: OB(O)c1ccccc1-c1ccccc1 ; CC1(C)c2ccccc2-c2ccc(Br)cc21 ; CC1(C)c2cc(Br)ccc2-c2ccc(-c3ccccc3-c3ccccc3)cc21 ; CC1(C)c2cc(Br)ccc2-c2cc3c4ccccc4c4ccccc4c3cc21 ; "FeCl3·CH3NO2" ; "Na2CO3·Pd(PPh3)4"]

A mixture of 35.2 g (100 mmol) of 2,7-dibromo-9,9-dimethyl-9H-fluorene (CAS: 28320-31-2), 21.8 g (110 mmol) of biphenyl-2-yl borate, 2.31 g (2 mmol) of Pd(PPh₃)₄, 75 mL of 2M Na₂CO₃, 150 mL of EtOH and 300 mL of toluene was degassed and placed under nitrogen, and then was heated at 100° C. for 12 hours. After a reaction was completed, the mixture was cooled to the room temperature. An organic layer was extracted with ethyl acetate and water and dried with anhydrous magnesium sulfate, a solvent was removed, and residues were purified through pipe column chromatography filled with silica gel to obtain a white-solid product 2-(biphenyl-2-yl)-7-bromo-9,9-dimethyl-9H-fluorene (26.8 g, 63.0 mmol, 63%).

In a 3000 mL three-necked flask which is degassed and filled with nitrogen, 26.8 g (60 mmol) of 2-(biphenyl-2-yl)-7-bromo-9,9-dimethyl-9H-fluorene was dissolved in anhydrous dichloromethane (1500 mL), then 97.5 g (600 mmol) of ferric chloride (III) was added, and a mixture was stirred for one hour. 500 mL of methanol was added into the mixture, an organic layer was separated, and a solvent was removed in vacuum. Residues were purified through pipe column chromatography (hexane-dichloromethane) filled with silica gel to obtain a white solid N-912A (12-bromo-10,10-dimethyl-10H-indeno[1,2-b]triphenylene, 10.7 g, 25.3 mmol, 40%).

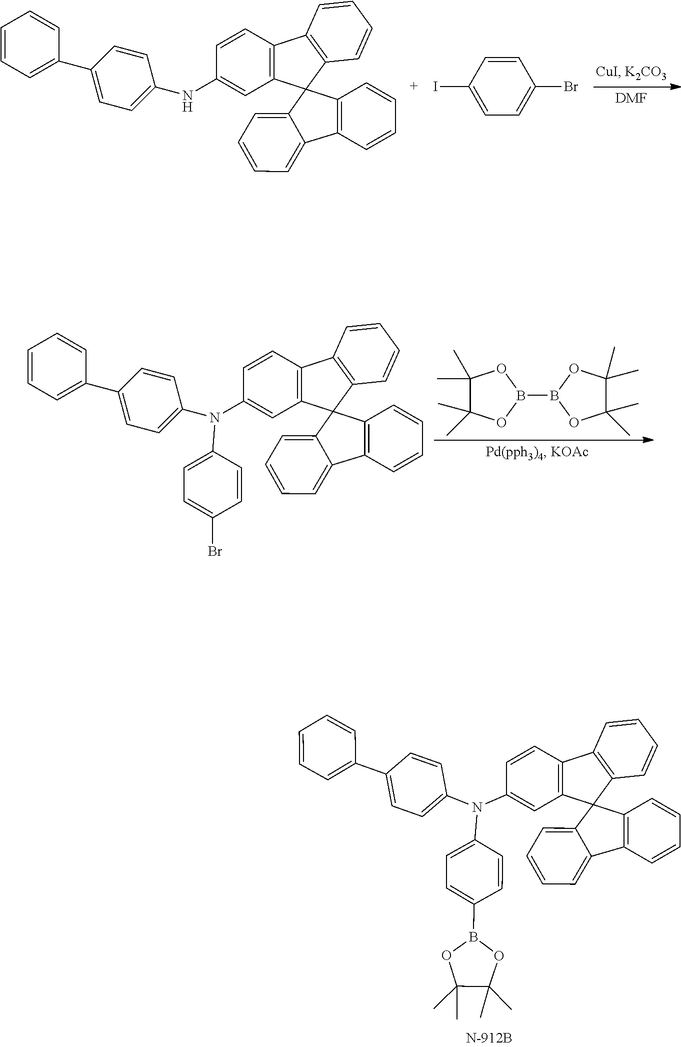

A mixture of 20 g (41.3 mmol) of N-(biphenyl-4-yl)-9,9′-spirobifluorene-2-amine (CAS: 1258514-95-2), 14 g (49.5 mmol) of 1-bromo-4-iodobenzene, 2.4 g (12.4 mmol) of copper iodide, 17.1 g (123.9 mmol) of potassium carbonate and 300 mL of DMF was refluxed overnight under nitrogen. After a reaction was finished, the mixture was then cooled to the room temperature. An organic layer was extracted with ethyl acetate and water and dried with anhydrous magnesium sulfate, a solvent was removed, and residues were purified through pipe column chromatography (hexane-dichloromethane) filled with silica gel to obtain 26.3 g (yield 43%) of a white-solid product.

A mixture of 10 g (15.6 mmol) of N-(biphenyl-4-yl)-N-(4-bromo-phenyl)-9,9′-spirobi[fluorene]-2-amine, 4.75 g (18.72 mmol) of bis(pinacolato)diboron, 0.18 g (0.156 mmol) of tetrakis(triphenylphosphine)palladium, 2 g (20.28 mmol) of potassium acetate and 300 mL of 1,4-dioxane was degassed and placed under nitrogen, and then was heated at 90° C. for 16 h. After a reaction was finished, the mixture was cooled to the room temperature. An organic layer was extracted with ethyl acetate and water and dried with anhydrous magnesium sulfate, a solvent was removed, and a product was purified through a pipe column with a mixture of hexane and ethyl as an eluent to obtain 8.77 g of a pale-yellow product N-912B (N-(biphenyl-4-yl)-N-(4-(4,4,5,5-tetramethyl-1,3,2-dioxaborolane-2-yl)phenyl)-9,9′-spirobifluorene-2-amine, yield 82%).

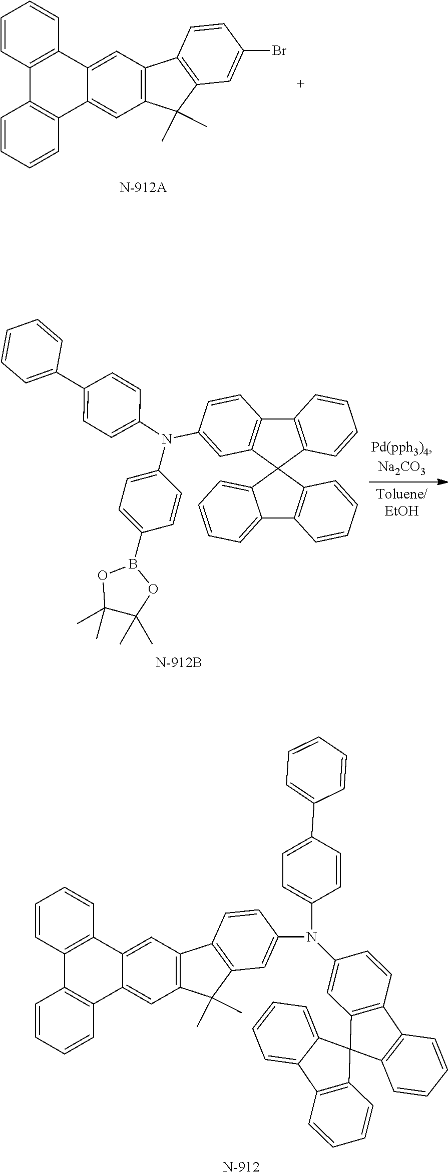 [SMILES: CC1(C)c2cc(Br)ccc2-c2cc3c4ccccc4c4ccccc4c3cc21 ; CC1(C)OB(c2ccc(N(c3ccc(-c4ccccc4)cc3)c3ccc4c(c3)C3(c5ccccc5-c5ccccc53)c3ccccc3-4)cc2)OC1(C)C ; CC1(C)c2cc(N(c3ccc(-c4ccccc4)cc3)c3ccc4c(c3)C3(c5ccccc5-c5ccccc53)c3ccccc3-4)ccc2-c2cc3c4ccccc4c4ccccc4c3cc21]

A mixture of 15 g (35.43 mmol) of 12-bromo-10,10-dimethyl-10H-indeno[1,2-b]triphenylene (N-912A), 29.1 g (42.51 mmol) of N-(biphenyl-4-yl)-N-(4-(4,4,5,5-tetramethyl-1,3,2-dioxaborolane-2-yl)phenyl)-9,9′-spirobifluorene-2-amine (N-912B), 0.41 g (0.35 mmol) of tetrakis(triphenylphosphine)palladium, 23 mL of 2M Na₂CO₃, 100 mL of EtOH and 200 mL of toluene was degassed and placed under nitrogen, and then was heated at 100° C. for 8 hours. After a reaction was finished, the mixture was cooled to the room temperature. An organic layer was extracted with dichloromethane and water and dried with anhydrous magnesium sulfate, a solvent was removed, and residues were purified through pipe column chromatography (hexane-dichloromethane) filled with silica gel to obtain 17.5 g of a yellow-solid product (N-912, yield 55%).

Elemental analysis: C₇₀H₄₇N; theoretical value: C, 93.20; H, 5.25; N, 1.55; measured value: C, 93.16; H, 5.27; N, 1.57; HRMS (ESI) m/z (M+): theoretical value: 901.37; measured value: 902.21.

### 9. Synthetic Route of N-895

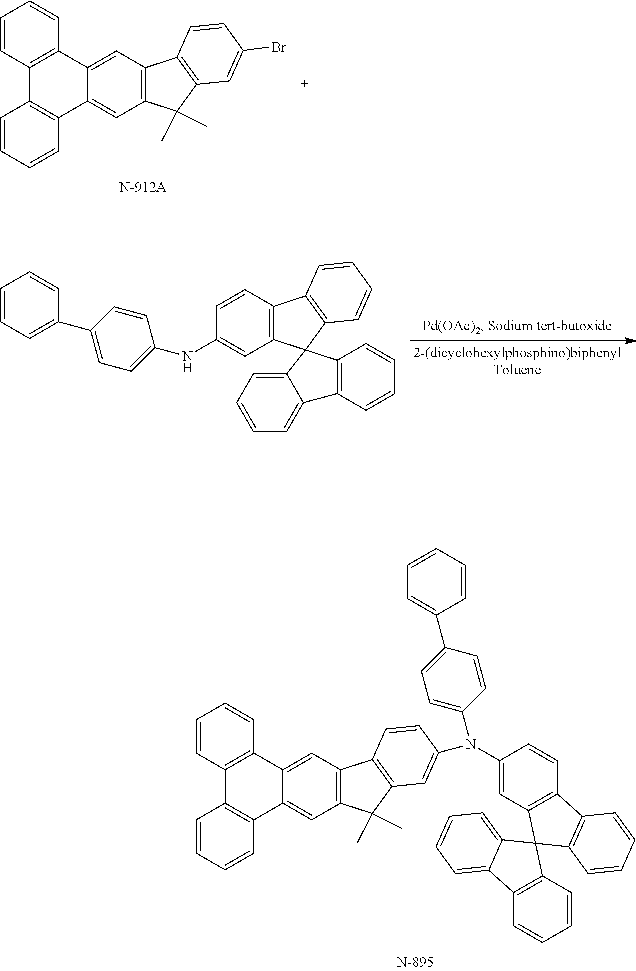 [SMILES: CC1(C)c2cc(Br)ccc2-c2cc3c4ccccc4c4ccccc4c3cc21 ; c1ccc(-c2ccc(Nc3ccc4c(c3)C3(c5ccccc5-c5ccccc53)c3ccccc3-4)cc2)cc1 ; CC1(C)c2cc(N(c3ccc(-c4ccccc4)cc3)c3ccc4c(c3)C3(c5ccccc5-c5ccccc53)c3ccccc3-4)ccc2-c2cc3c4ccccc4c4ccccc4c3cc21]

A mixture of 5 g (11.8 mmol) of 12-bromo-10,10-dimethyl-10H-indeno[1,2-b]triphenylene (N-912A), 6.8 g (14.1 mmol) of N-(biphenyl-4-yl)-9,9′-spirobifluorene-2-amine (CAS: 1258514-95-2), 0.03 g (0.11 mmol) of palladium acetate (II), 0.04 g (0.11 mmol) of 2-(dicyclohexylphosphino)biphenyl, 1.7 g (17.7 mmol) of sodium tert-butoxide and 100 mL of toluene was refluxed overnight under nitrogen. After a reaction was finished, the mixture was then cooled to the room temperature. An organic layer was extracted with dichloromethane and water and dried with anhydrous magnesium sulfate, a solvent was removed, and residues were purified through pipe column chromatography (hexane-dichloromethane) filled with silica gel to obtain 5.8 g of a yellow-solid product (N-895, yield 60%).

Elemental analysis: C₆₄H₄₃N; theoretical value: C, 93.06; H, 5.25; N, 1.70; measured value: C, 93.02; H, 5.25; N, 1.73; HRMS (ESI) m/z (M+): theoretical value: 825.34; measured value: 826.19.

### 10. Synthetic Route of N-899, N-901, N-906 and N-918

Synthesis conditions were the same as those of N-895, and the difference was that the structures and yields of a raw material N-nA, a raw material N-nB and products were different, specifically as shown in Table 7 below; and the elemental analysis results of the prepared compounds were as shown in Table 8.

TABLE 7

| Raw material N-nA | Raw material N-nB | |
|---|---|---|
| 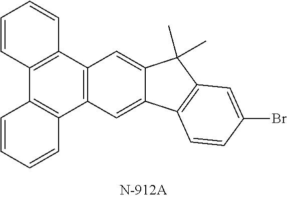 [SMILES: CC1(C)c2cc(Br)ccc2-c2cc3c4ccccc4c4ccccc4c3cc21] | 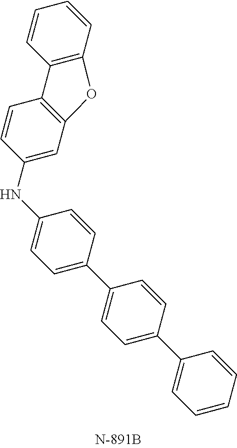 [SMILES: c1ccc(-c2ccc(-c3ccc(Nc4ccc5c(c4)oc4ccccc45)cc3)cc2)cc1] | |
| 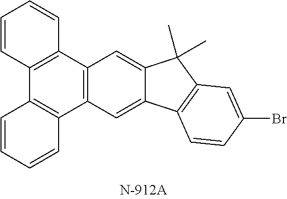 [SMILES: CC1(C)c2cc(Br)ccc2-c2cc3c4ccccc4c4ccccc4c3cc21] | 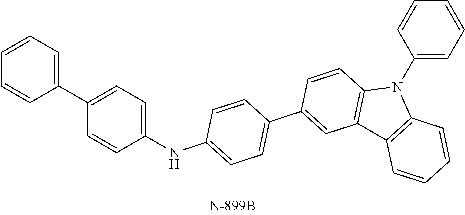 [SMILES: c1ccc(-c2ccc(Nc3ccc(-c4ccc5c(c4)c4ccccc4n5-c4ccccc4)cc3)cc2)cc1] | |
| 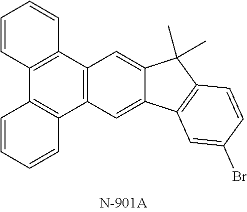 [SMILES: CC1(C)c2ccc(Br)cc2-c2cc3c4ccccc4c4ccccc4c3cc21] | 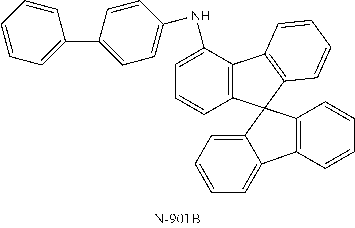 [SMILES: c1ccc(-c2ccc(Nc3cccc4c3-c3ccccc3C43c4ccccc4-c4ccccc43)cc2)cc1] | |
| 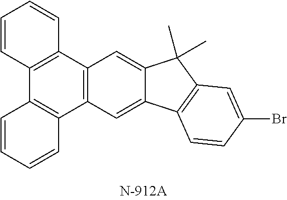 [SMILES: CC1(C)c2cc(Br)ccc2-c2cc3c4ccccc4c4ccccc4c3cc21] | 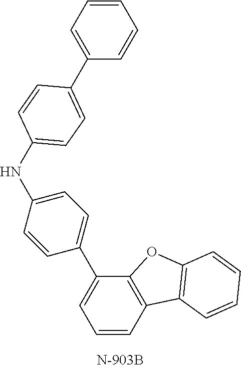 [SMILES: c1ccc(-c2ccc(Nc3ccc(-c4cccc5c4oc4ccccc45)cc3)cc2)cc1] | |
| 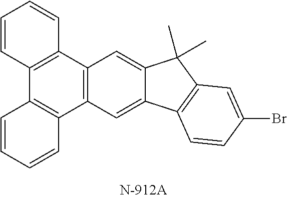 [SMILES: CC1(C)c2cc(Br)ccc2-c2cc3c4ccccc4c4ccccc4c3cc21] | 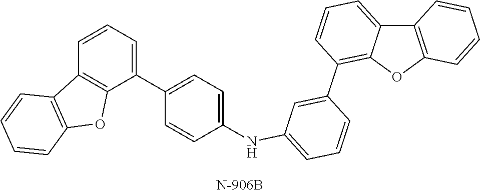 [SMILES: c1cc(Nc2ccc(-c3cccc4c3oc3ccccc34)cc2)cc(-c2cccc3c2oc2ccccc23)c1] | |
| 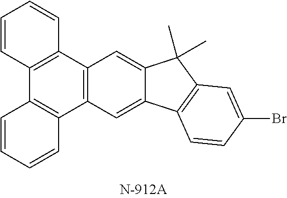 [SMILES: CC1(C)c2cc(Br)ccc2-c2cc3c4ccccc4c4ccccc4c3cc21] | 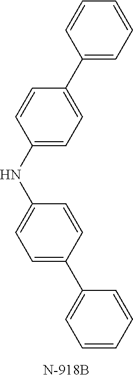 [SMILES: c1ccc(-c2ccc(Nc3ccc(-c4ccccc4)cc3)cc2)cc1] | |
| 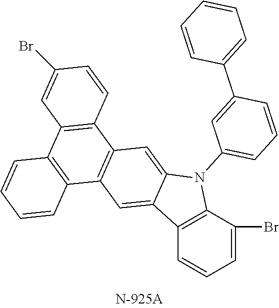 [SMILES: Brc1ccc2c(c1)c1ccccc1c1cc3c4cccc(Br)c4n(-c4cccc(-c5ccccc5)c4)c3cc21] | 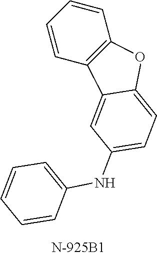 [SMILES: c1ccc(Nc2ccc3oc4ccccc4c3c2)cc1] | |
| | 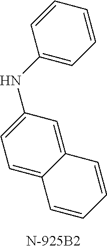 [SMILES: c1ccc(Nc2ccc3ccccc3c2)cc1] | |
| | Products | Yield % |
| | 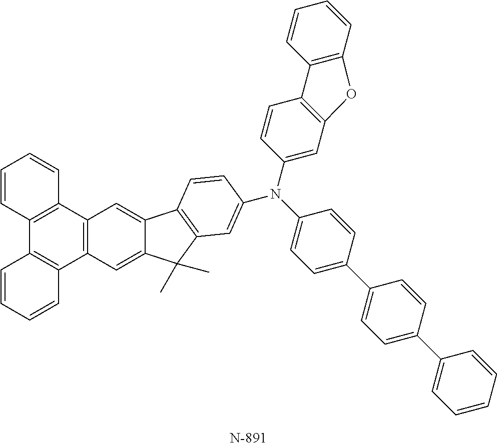 [SMILES: CC1(C)c2cc(N(c3ccc(-c4ccc(-c5ccccc5)cc4)cc3)c3ccc4c(c3)oc3ccccc34)ccc2-c2cc3c4ccccc4c4ccccc4c3cc21] | 63 |
| | 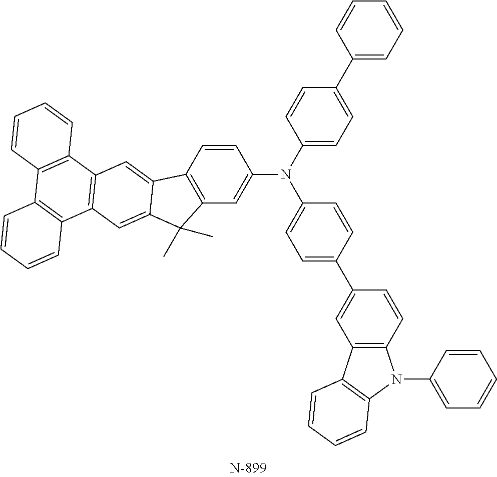 [SMILES: CC1(C)c2cc(N(c3ccc(-c4ccccc4)cc3)c3ccc(-c4ccc5c(c4)c4ccccc4n5-c4ccccc4)cc3)ccc2-c2cc3c4ccccc4c4ccccc4c3cc21] | 61 |
| | 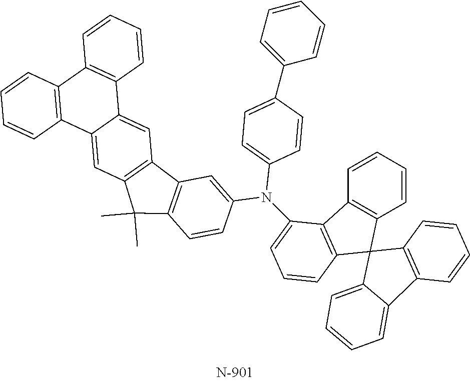 [SMILES: CC1(C)c2ccc(N(c3ccc(-c4ccccc4)cc3)c3cccc4c3-c3ccccc3C43c4ccccc4-c4ccccc43)cc2-c2cc3c4ccccc4c4ccccc4c3cc21] | 55 |
| | 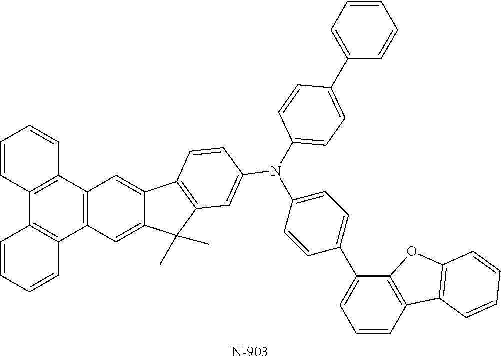 [SMILES: CC1(C)c2cc(N(c3ccc(-c4ccccc4)cc3)c3ccc(-c4cccc5c4oc4ccccc45)cc3)ccc2-c2cc3c4ccccc4c4ccccc4c3cc21] | 62 |
| | 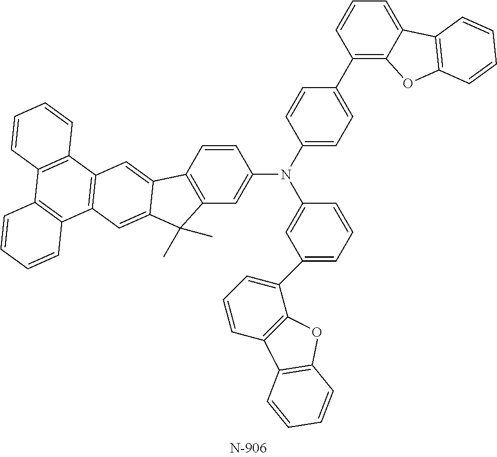 [SMILES: CC1(C)c2cc(N(c3ccc(-c4cccc5c4oc4ccccc45)cc3)c3cccc(-c4cccc5c4oc4ccccc45)c3)ccc2-c2cc3c4ccccc4c4ccccc4c3cc21] | 46 |
| | 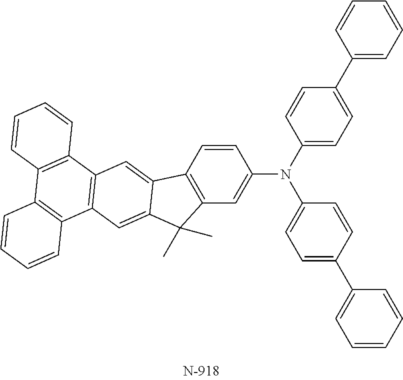 [SMILES: CC1(C)c2cc(N(c3ccc(-c4ccccc4)cc3)c3ccc(-c4ccccc4)cc3)ccc2-c2cc3c4ccccc4c4ccccc4c3cc21] | 40 |
| | 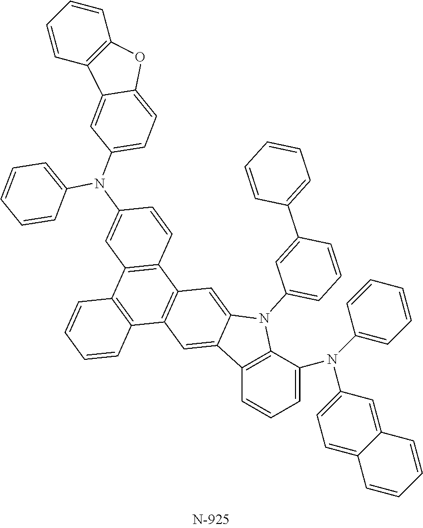 [SMILES: c1ccc(-c2cccc(-n3c4cc5c6ccc(N(c7ccccc7)c7ccc8oc9ccccc9c8c7)cc6c6ccccc6c5cc4c4cccc(N(c5ccccc5)c5ccc6ccccc6c5)c43)c2)cc1] | 32 |

TABLE 8

| | | | HRMS (ESI) m/z | |
|---|---|---|---|---|
| | Elemental analysis | | [M + H]+ | |
| Compounds | Theoretical | Measured | Theoretical | Measured |
| N-891 | C, 90.81; H, 5.21; N, 1.86; O, | C, 90.79; H, 5.22; N, | 753.30 | 754.02 |
| | 2.12; | 1.87; | | |
| N-899 | C, 91.27; H, 5.35; N, 3.38; | C, 91.25; H, 5.36; N, | 828.35 | 829.61 |
| | | 3.39; | | |
| N-901 | C, 93.06; H, 5.25; N, 1.70; | C, 93.03; H, 5.26; N, | 825.34 | 826.55 |
| | | 1.71; | | |
| N-903 | C, 90.81; H, 5.21; N, 1.86; O, | C, 90.79; H, 5.21; N, | 753.30 | 754.21 |
| | 2.12; | 1.88; | | |
| N-906 | C, 89.65; H, 4.90; N, 1.66; O, | C, 89.63; H, 4.91; N, | 843.31 | 844.77 |
| | 3.79; | 1.67; | | |
| N-918 | C, 92.27; H, 5.62; N, 2.11; | C, 92.24; H, 5.63; N, | 663.29 | 664.36 |
| | | 2.13; | | |
| N-925 | C, 89.05; H, 4.80; N, 4.45; O, | C, 89.07; H, 4.81; N, | 943.36 | 944.18 |
| | 1.69; | 4.43; | | |

### Example 6

An organic electroluminescent device structurally consists of, sequentially from bottom to top: a substrate with an anode layer, a hole injection layer (HIL), a hole transport layer (HTL), a light emitting layer (EML), an electron transport layer (ETL), an electron injection layer (EIL) and a cathode. A specific preparation process is as follows:

(1) Substrate cleaning: a glass substrate coated with transparent ITO was subjected to ultrasonic treatment in a water-based cleaning agent (ingredients and concentrations of the water-based cleaning agent: a glycol solvent ≤10 wt %, triethanolamine ≤1 wt %), rinsed in deionized water, subjected to ultrasonic oil removal in an acetone:ethanol mixed solvent (volume ratio 1:1), baked in a clean environment to completely remove moisture, and then washed with ultraviolet light and ozone.

(2) Evaporation of hole injection layer:

The above glass substrate with the anode layer was placed in a vacuum cavity, the vacuum cavity was vacuumized to 1×10⁻⁶ to 2×10⁻⁴ Pa, and a mixture of NDP-9 and HT was evaporated in vacuum on a membrane of the above anode layer as a hole injection layer, where a mass ratio of NDP-9 to HT was 3:97, and an evaporation thickness was 10 nm; where structures of the NDP-9 and the HT were as follows:

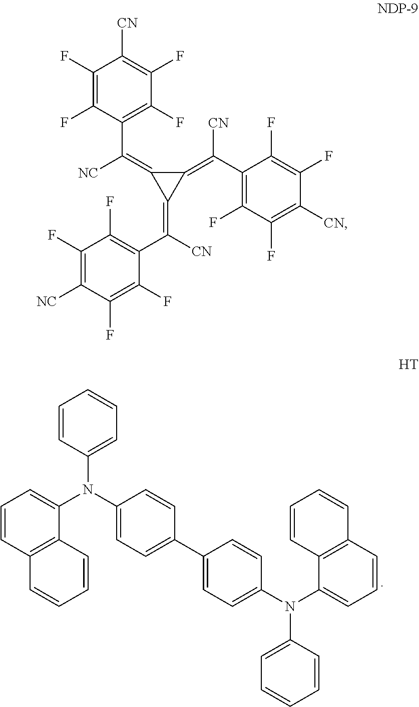 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(N(c2ccc(-c3ccc(N(c4ccccc4)c4cccc5ccccc45)cc3)cc2)c2cccc3ccccc23)cc1]

(3) A hole transport layer (a material being HT) was evaporated on the hole injection layer, where an evaporation membrane thickness was 80 nm.

(4) A light emitting layer was evaporated on the hole transport layer, and a specific preparation method was as follows: a light emitting host material and a dopant material (piq)₂Ir(acac) were evaporated in vacuum in a manner of co-evaporation, where the composition of the host material and the dopant material was as shown in Table 9.

TABLE 9

| No. | EML materials |
|---|---|
| Device | Mass ratio of N-4:M-17:(piq)₂Ir(acac) = 47.5:47.5:5 |
| NM1-1 | |
| Device | Mass ratio of N-46:M-76:(piq)₂Ir(acac) = 47.5:47.5:5 |
| NM2-2 | |
| Device | Mass ratio of N-369:M-108:(piq)₂Ir(acac) = 47.5:47.5:5 |
| NM3-3 | |
| Device | Mass ratio of N-423:M-145:(piq)₂Ir(acac) = 47.5:47.5:5 |
| NM4-4 | |
| Device | Mass ratio of N-439:M-253:(piq)₂Ir(acac) = 47.5:47.5:5 |
| NM5-5 | |
| Device | Mass ratio of N-685:M-296:(piq)₂Ir(acac) = 47.5:47.5:5 |
| NM6-6 | |
| Device | Mass ratio of N-728:M-296:(piq)₂Ir(acac) = 47.5:47.5:5 |
| NM7-6 | |
| Device | Mass ratio of N-860:M-308:(piq)₂Ir(acac) = 47.5:47.5:5 |
| NM8-7 | |
| Device | Mass ratio of N-891:M-365:(piq)₂Ir(acac) = 47.5:47.5:5 |
| NM9-8 | |
| Device | Mass ratio of N-903:M-371:(piq)₂Ir(acac) = 47.5:47.5:5 |
| NM10-9 | |
| Device | Mass ratio of N-912:M-381:(piq)₂Ir(acac) = 47.5:47.5:5 |
| NM11-10 | |
| Device | Mass ratio of N-925:M-17:(piq)₂Ir(acac) = 47.5:47.5:5 |
| NM12-1 | |
| Device | Mass ratio of N-4:M-394:(piq)₂Ir(acac) = 47.5:47.5:5 |
| NM1-11 | |
| Device | Mass ratio of N-4:M-412:(piq)₂Ir(acac) = 47.5:47.5:5 |
| NM1-12 | |
| Device | Mass ratio of N-4:M-423:(piq)₂Ir(acac) = 47.5:47.5:5 |
| NM1-13 | |
| Device | Mass ratio of N-4:M-442:(piq)₂Ir(acac) = 47.5:47.5:5 |
| NM1-14 | |
| Device | Mass ratio of N-4:M-450:(piq)₂Ir(acac) = 47.5:47.5:5 |
| NM1-15 | |
| Device | Mass ratio of N-4:M-460:(piq)₂Ir(acac) = 47.5:47.5:5 |
| NM1-16 | |
| Device | Mass ratio of N-4:M-461:(piq)₂Ir(acac) = 47.5:47.5:5 |
| NM1-17 | |
| Device | Mass ratio of N-4:M-480:(piq)₂Ir(acac) = 47.5:47.5:5 |
| NM1-18 | |
| Device | Mass ratio of N-4:M-502:(piq)₂Ir(acac) = 47.5:47.5:5 |
| NM1-19 | |
| Device | Mass ratio of N-4:M-520:(piq)₂Ir(acac) = 47.5:47.5:5 |
| NM1-20 | |
| Device | Mass ratio of N-4:M-526:(piq)₂Ir(acac) = 47.5:47.5:5 |
| NM1-21 | |
| Device | Mass ratio of N-4:M-537:(piq)₂Ir(acac) = 47.5:47.5:5 |

NM1-22

| | |
|---|---|
| Device | Mass ratio of N-4:M-548:(piq)₂Ir(acac) = 47.5:47.5:5 |
| NM1-23 | |
| Device | Mass ratio of N-4:M-562:(piq)₂Ir(acac) = 47.5:47.5:5 |
| NM1-24 | |
| Device | Mass ratio of N-4:M-572:(piq)₂Ir(acac) = 47.5:47.5:5 |
| NM1-25 | |
| Device | Mass ratio of N-4:M-579:(piq)₂Ir(acac) = 47.5:47.5:5 |
| NM1-26 | |
| Device | Mass ratio of N-4:M-584:(piq)₂Ir(acac) = 47.5:47.5:5 |
| NM1-27 | |
| Device | Mass ratio of N-4:M-589:(piq)₂Ir(acac) = 47.5:47.5:5 |
| NM1-28 | |
| Device | Mass ratio of N-4:M-599:(piq)₂Ir(acac) = 47.5:47.5:5 |
| NM1-29 | |
| Device | Mass ratio of N-4:M-610:(piq)₂Ir(acac) = 47.5:47.5:5 |
| NM1-30 | |
| Device | Mass ratio of N-4:M-611:(piq)₂Ir(acac) = 47.5:47.5:5 |
| NM1-31 | |
| Device | Mass ratio of N-4:M-17:(piq)₂Ir(acac) = 38:57:5 |
| NM1-1′ | |
| Device | Mass ratio of N-685:M-296:(piq)₂Ir(acac) = 38:57:5 |
| NM6-6′ | |
| Device | Mass ratio of N-423:M-381:(piq)₂Ir(acac) = 47.5:47.5:5 |
| NM4-10 | |
| Device M1 | Mass ratio of M-17:(piq)₂Ir(acac) = 95:5 |
| Device M2 | Mass ratio of M-76:(piq)₂Ir(acac) = 95:5 |
| Device M3 | Mass ratio of M-108:(piq)₂Ir(acac) = 95:5 |
| Device M4 | Mass ratio of M-145:(piq)₂Ir(acac) = 95:5 |
| Device M5 | Mass ratio of M-253:(piq)₂Ir(acac) = 95:5 |
| Device M6 | Mass ratio of M-296:(piq)₂Ir(acac) = 95:5 |
| Device M7 | Mass ratio of M-308:(piq)₂Ir(acac) = 95:5 |
| Device M8 | Mass ratio of M-365:(piq)₂Ir(acac) = 95:5 |
| Device M9 | Mass ratio of M-371:(piq)₂Ir(acac) = 95:5 |
| Device M10 | Mass ratio of M-381:(piq)₂Ir(acac) = 95:5 |
| Device M1C | Mass ratio of M-17:CBP:(piq)₂Ir(acac) = 47.5:47.5:5 |

A total evaporation membrane thickness was 38 nm; where the structure of (piq)₂Ir(acac) was as follows:

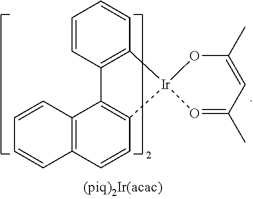

(5) An electron transport layer was evaporated on the light emitting layer, and a specific preparation method was as follows: materials of the electron transport layer were evaporated in vacuum in a manner of co-evaporation, the materials were ET-1 and LiQ with a mass ratio of 1:1, and a total evaporation membrane thickness was 30 nm, where the structures of the ET-1 and the LiQ were as follows:

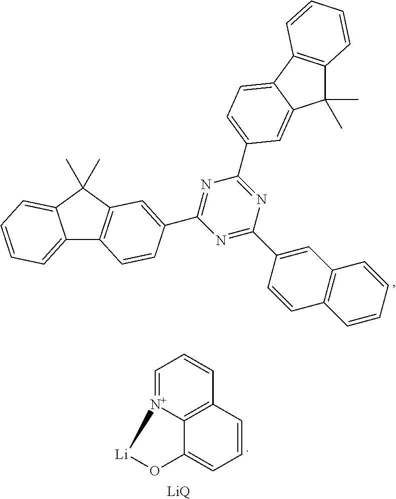

(6) An electron injection layer was evaporated in vacuum on the electron transport layer, a material of the electron injection layer was LiQ, and a total evaporation membrane thickness was 1 nm.

(7) Al was evaporated on the electron injection layer, where a total evaporation membrane thickness was 80 nm.

### Comparative Example 1

The difference between this comparative example and Example 6 is that during the preparation of a device, the composition of the host material used in step (5) is different, specifically as shown in Table 10; and other steps and parameter conditions are the same as those in Example 6.

TABLE 10

| No. | EML materials |
|---|---|
| Device N1 | Mass ratio of N-4:(piq)₂Ir(acac) = 95:5 |
| Device N1 | Mass ratio of N-4:(piq)₂Ir(acac) = 95:5 |
| Device N6 | Mass ratio of N-685:(piq)₂Ir(acac) = 95:5 |
| Device C | Mass ratio of CBP:(piq)₂Ir(acac) = 95:5 |
| Device RC | Mass ratio of REF-1:CBP:(piq)₂Ir(acac) = 47.5:47.5:5 |
| Device N1C | Mass ratio of N-4:CBP:(piq)₂Ir(acac) = 47.5:47.5:5 |

In the above table, the structures of CBP (4,4′-bis(N-carbazole)-1,1′-biphenyl, CAS: 58328-31-7) and REF-1 (CAS: 1070884-53-5) are as follows:

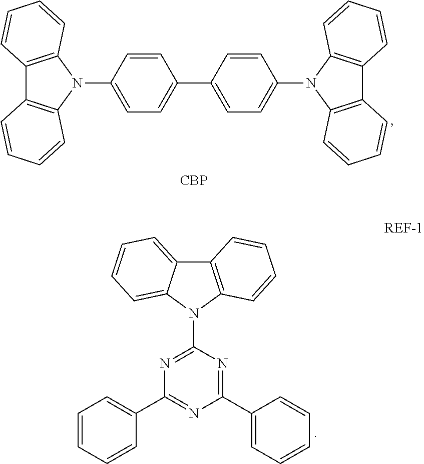 [SMILES: c1ccc2c(c1)c1ccccc1n2-c1ccc(-c2ccc(-n3c4ccccc4c4ccccc43)cc2)cc1 ; "CBP" ; c1ccc(-c2nc(-c3ccccc3)nc(-n3c4ccccc4c4ccccc43)n2)cc1]

### Experimental Example

The devices prepared in Example 6 and Comparative example 1 are adopted for performance testing, and specific testing conditions are as follows:

The current, voltage, brightness, emission spectrum and other characteristics of the devices were synchronously tested using a PR 650 spectral scanning brightness meter and a Keithley K 2400 digital source meter system;
testing condition for optical-electrical characteristic: a current density was 10 mA/cm²;
service life testing: a current density was 50 mA/cm², and time was recorded (in hours) when the device brightness was lowered to 95% of the original brightness.

Results of the above device performance testing were as shown in Table 11 and Table 12.

TABLE 11

| | Driving | Current efficiency | Service life |
|---|---|---|---|
| | voltage (V) | (Cd/A) | T95 (hrs) |
| Device N1 | 4.00 | 16.31 | 110.8 |
| Device N1 | 4.00 | 16.31 | 110.8 |
| Device M1 | 3.72 | 25.11 | 140.2 |
| Device NM1-1 | 3.29 | 27.59 | 278.7 |
| Device NM1-1′ | 3.24 | 28.79 | 294.7 |
| Device N6 | 4.04 | 15.37 | 96.8 |
| Device M6 | 3.78 | 24.77 | 136.7 |
| Device NM6-6 | 3.25 | 27.66 | 284.5 |
| Device NM6-6′ | 3.20 | 29.44 | 299.5 |
| Device C | 4.80 | 5.00 | 5.0 |
| Device M1 | 3.72 | 25.11 | 140.2 |
| Device M1C | 3.87 | 23.43 | 151.0 |

TABLE 12

| | Driving | Current efficiency | Service life |
|---|---|---|---|
| | voltage (V) | (Cd/A) | T95 (hrs) |
| Device NM1-1 | 3.29 | 27.59 | 278.7 |
| Device NM2-2 | 3.37 | 25.45 | 260.4 |
| Device NM3-3 | 3.31 | 26.89 | 277.4 |
| Device NM4-4 | 3.22 | 28.47 | 290.4 |
| Device NM5-5 | 3.42 | 24.98 | 279.8 |
| Device NM6-6 | 3.25 | 27.66 | 284.5 |
| Device NM7-6 | 3.44 | 24.00 | 263.4 |
| Device NM8-7 | 3.27 | 27.11 | 288.9 |
| Device NM9-8 | 3.33 | 26.55 | 279.6 |
| Device NM10-9 | 3.34 | 25.78 | 270.8 |
| Device NM11-10 | 3.36 | 25.94 | 272.1 |
| Device NM12-1 | 3.40 | 24.97 | 268.4 |
| Device NM1-11 | 3.41 | 22.57 | 253.4 |
| Device NM1-12 | 3.39 | 23.47 | 263.7 |
| Device NM1-13 | 3.20 | 31.48 | 312.7 |
| Device NM1-14 | 3.21 | 30.39 | 307.8 |
| Device NM1-15 | 3.19 | 32.45 | 324.1 |
| Device NM1-16 | 3.37 | 24.16 | 259.8 |
| Device NM1-17 | 3.22 | 29.64 | 304.3 |
| Device NM1-18 | 3.35 | 22.65 | 253.7 |
| Device NM1-19 | 3.25 | 29.52 | 310.6 |
| Device NM1-20 | 3.38 | 22.63 | 283.2 |
| Device NM1-21 | 3.22 | 29.15 | 304.9 |
| Device NM1-22 | 3.19 | 31.86 | 326.1 |
| Device NM1-23 | 3.32 | 24.12 | 263.7 |
| Device NM1-24 | 3.26 | 25.45 | 296.2 |
| Device NM1-25 | 3.26 | 24.58 | 273.6 |
| Device NM1-26 | 3.23 | 29.36 | 301.5 |
| Device NM1-27 | 3.35 | 23.67 | 271.1 |
| Device NM1-28 | 3.17 | 32.45 | 321.6 |
| Device NM1-29 | 3.20 | 30.65 | 316.4 |
| Device NM1-30 | 3.30 | 23.57 | 292.7 |
| Device NM1-31 | 3.24 | 25.36 | 298.9 |
| Device NM1-1′ | 3.24 | 28.79 | 294.7 |
| Device NM6-6′ | 3.20 | 29.44 | 299.5 |
| Device NM4-10 | 3.27 | 27.99 | 285.4 |
| Device M1 | 3.72 | 25.11 | 140.2 |
| Device M2 | 3.79 | 25.69 | 139.7 |
| Device M3 | 3.70 | 26.11 | 148.2 |
| Device M4 | 3.73 | 25.81 | 140.3 |
| Device M5 | 3.85 | 23.44 | 134.5 |
| Device M6 | 3.78 | 24.77 | 136.7 |
| Device M7 | 3.79 | 23.98 | 139.5 |
| Device M8 | 3.71 | 25.61 | 144.5 |
| Device M9 | 3.88 | 23.44 | 149.3 |
| Device M10 | 3.79 | 24.56 | 146.2 |
| Device M1C | 3.87 | 23.43 | 151.0 |
| Device C | 4.80 | 5.00 | 5.0 |
| Device RC | 4.27 | 19.76 | 48.0 |
| Device N1C | 4.65 | 17.54 | 159.6 |

It can be known from the comparison of data corresponding to the example and the comparative example in Table 12 that, the novel compound M developed in the present disclosure has obviously more excellent performance compared to CBP, REF-1, etc. disclosed in the prior art, and a device prepared from the compound can have a lower lighting voltage, i.e., the lower driving voltage in Table 11 and Table 12.

It can be known from the data of the device NM in Table 12 above that, when the organic electroluminescent material is used as an organic functional layer material, the synergistic effect of the compound N and the compound M can significantly improve the performance of the device compared to the matching of other compounds with similar structures that have been disclosed. Please refer to a device RC in which REF-1 and CBP match with each other, a device N1C in which N-4 and CBP match with each other, a device M1C in which M1 and CBP match with each other, etc. At the same time, the above effects can also be known through comparison of the data in Table 11 above, the compound N and the compound M are selected to match with each other as the host material in the light emitting material, which can achieve a more significant synergistic effect. Compared to using the compound N or the compound M alone, or matching with other compounds as the organic electroluminescent host material, the combination of the compound N and the compound M has a significantly lower lighting voltage, which significantly improves the light emitting efficiency of the device and significantly prolongs the service life of the device. The finally prepared device has a lower driving voltage (3.44 V or below), higher current efficiency (24 Cd/A or above), and a longer service life (260 h or above).

As can be seen from the above, the matching of the compound M and the compound N developed in the present disclosure can significantly improve the carrier injection efficiency, reduce the interlayer energy level difference, balance the electron and hole transport rates, effectively improve the efficiency of an organic electroluminescent diode and prolong the service life of the organic electroluminescent diode. Such material is suitable not only as a light emitting host material, especially for a red light host material, a hole transport material and an electron blocking material, but also as an electron transport material and a hole blocking material, which greatly improves the light emitting efficiency of the device and prolongs the service life of the device. The combination of such compounds can also be used in the field of organic electroluminescent display. Specifically, the combination of such compounds can be used as a hole injection material or a hole transport material in an organic electroluminescent display, as well as a light emitting host material or a light emitting material in a fluorescent device.

Obviously, the above examples are only for the purpose of clearly illustrating the instances provided, rather than limiting the embodiments. For those of ordinary skill in the art, other different forms of changes or variations can be made based on the above explanation. It is not necessary and impossible to exhaustively list all embodiments here. The obvious changes or variations arising from this are still within the scope of protection of the present disclosure.

1. An organic electroluminescent compound, being a compound M having a structure shown in Formula (2), wherein Formula (2) is: 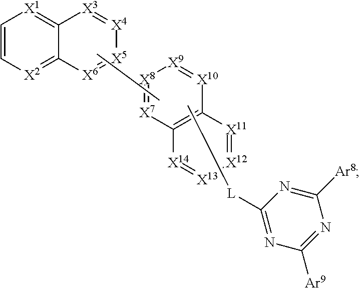
in Formula (2), X¹-X¹⁴ are each independently selected from N or CR⁸, and R⁸ is selected from hydrogen or deuterium;
L is selected from a connecting bond, substituted or unsubstituted C6-C30 arylenyl, and substituted or unsubstituted C3-C30 heteroarylenyl;
Ar⁸-Ar⁹ are each independently selected from substituted or unsubstituted C6-C30 aryl and substituted or unsubstituted C3-C30 heteroaryl;
substituents in the substituted C6-C30 arylenyl, the substituted C3-C30 heteroarylenyl, the substituted C6-C30 aryl and the substituted C3-C30 heteroaryl are selected from one of or a combination of two of deuterium, halogen, cyano, C1-C6 alkyl, C3-C30 cycloalkyl, C6-C30 aryl, and C3-C30 heteroaryl; and
when X¹-X¹⁴ are all CR⁸, Ar⁸-Ar⁹ are not substituted or unsubstituted naphthyl at the same time.

2. The organic electroluminescent compound according to claim 1, wherein in Formula (2), any one of X¹-X¹⁴ is selected from N, and the remaining is CR⁸; or any one of X¹-X⁶ is selected from N, the remaining is CR⁸, any one of X⁷-X¹⁴ is selected from N, and the remaining is CR⁸; or, X¹-X¹⁴ are all selected from CR⁸; when a plurality of R⁸ are present, R⁸ is each present independently, and the plurality of R⁸ can be the same or different; R⁸ is defined the same as that in claim 1; and
Ar⁸-Ar⁹ are each independently selected from hydrogen, deuterium and the following substituted or unsubstituted groups: phenyl, naphthyl, biphenyl, triphenyl, phenanthryl, fluoranthenyl, triphenylenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, benzothienyl, dibenzothienyl, carbazolyl, benzocarbazolyl, and dibenzocarbazolyl.

3. The organic electroluminescent compound according to claim 1, wherein Ar⁸-Ar⁹ are each independently selected from hydrogen, deuterium and the following substituted or unsubstituted groups: phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, naphthobenzofuryl, benzothienyl, dibenzothienyl, naphthobenzothienyl, carbazolyl, benzocarbazolyl, and dibenzocarbazolyl.

4. The organic electroluminescent compound according to claim 1, wherein the structure of the compound M is as shown in any one of Formula 2-1 to Formula 2-5, and the compound M is preferably the structure shown in Formula 2-4 or Formula 2-5; wherein:

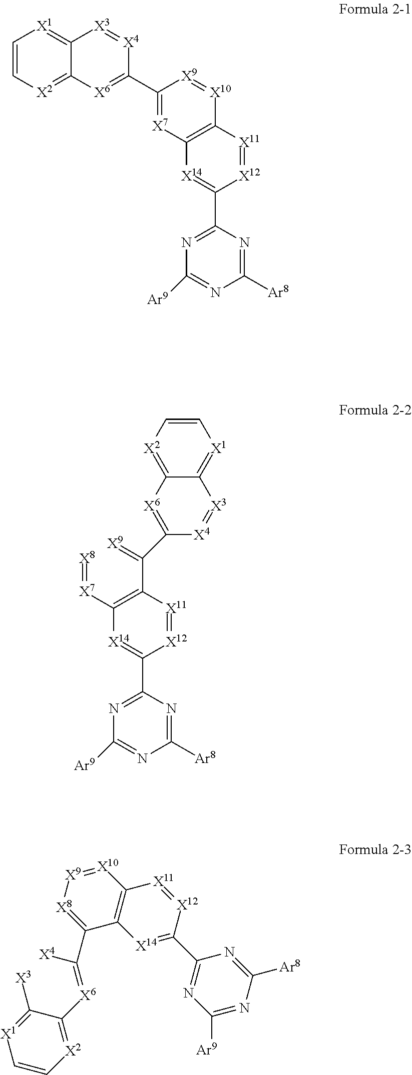

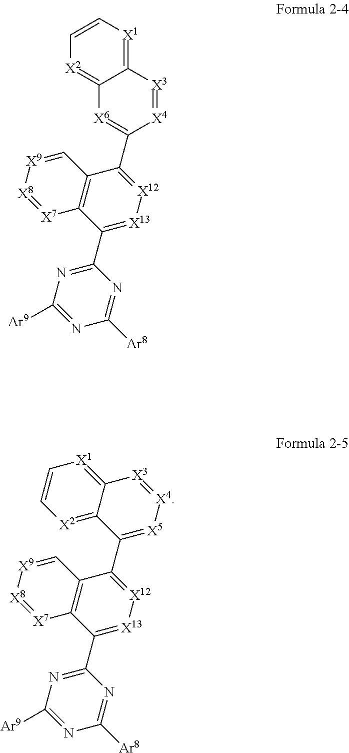

5. The organic electroluminescent compound according to claim 1, wherein the structure of the compound M is as shown in any one of Formula 2-6 to Formula 2-22, and preferably, the compound M is the structure shown in Formula 2-6, any one of Formula 2-8 to Formula 2-20, Formula 2-22, Formula 2-23 or any one of Formula 2-25 to Formula 2-28, wherein: 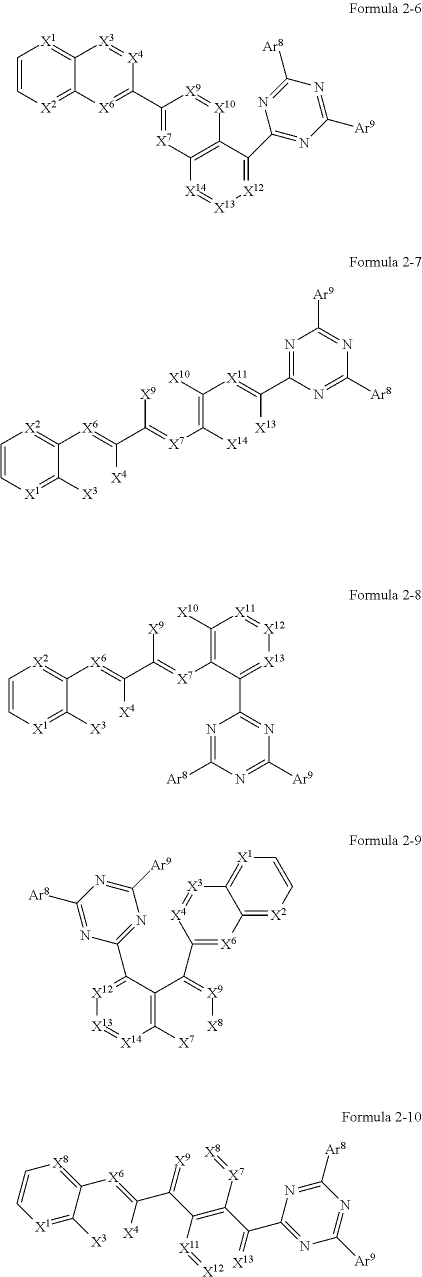 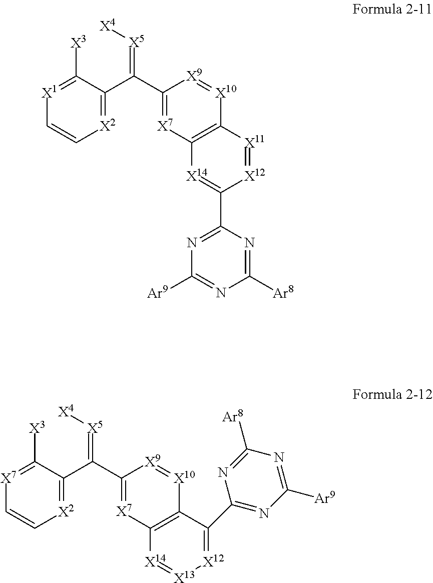 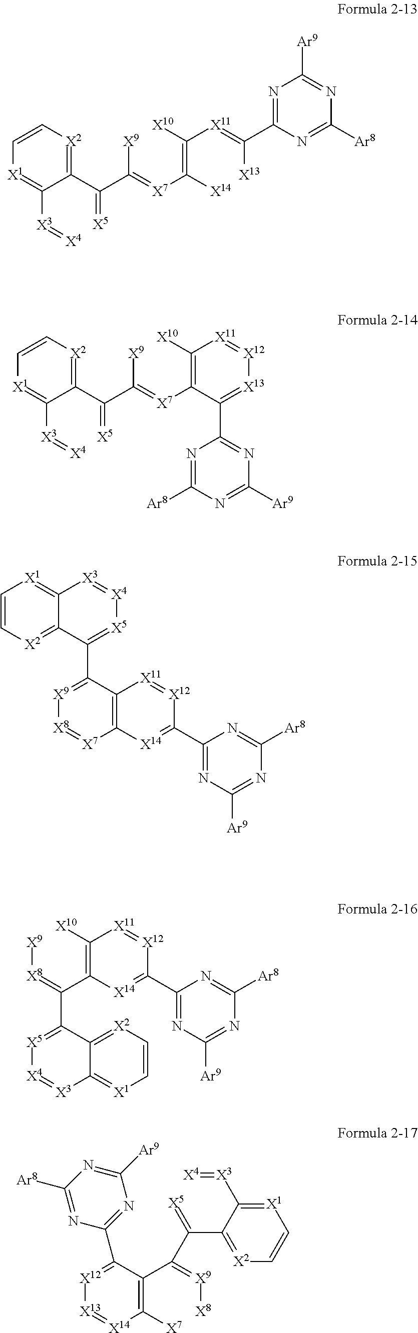 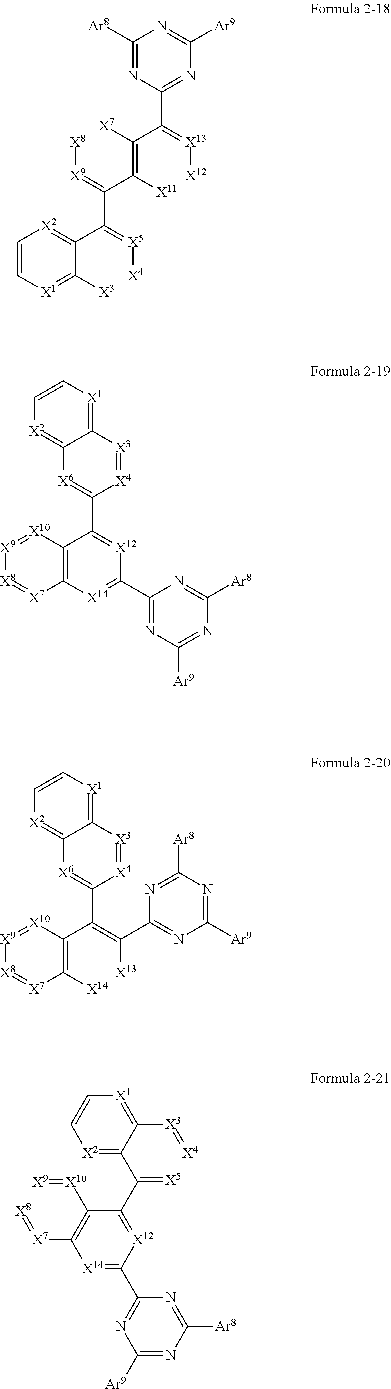 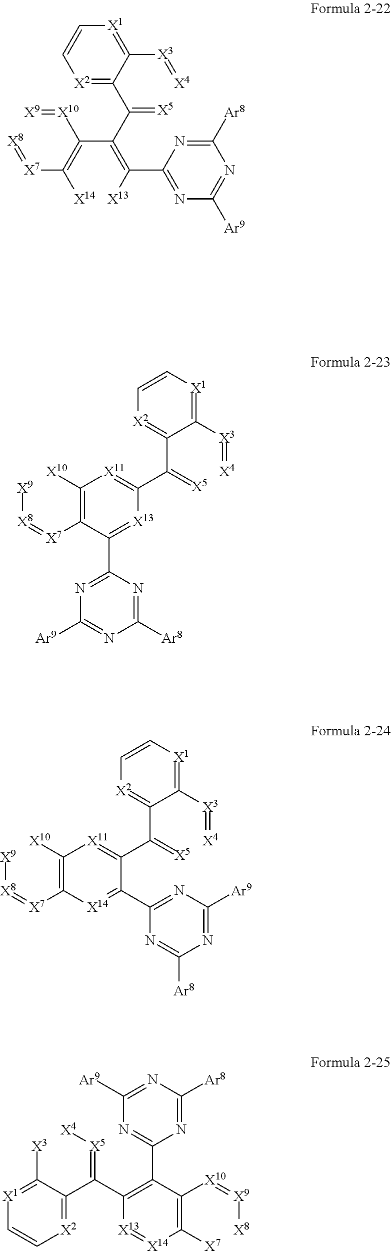 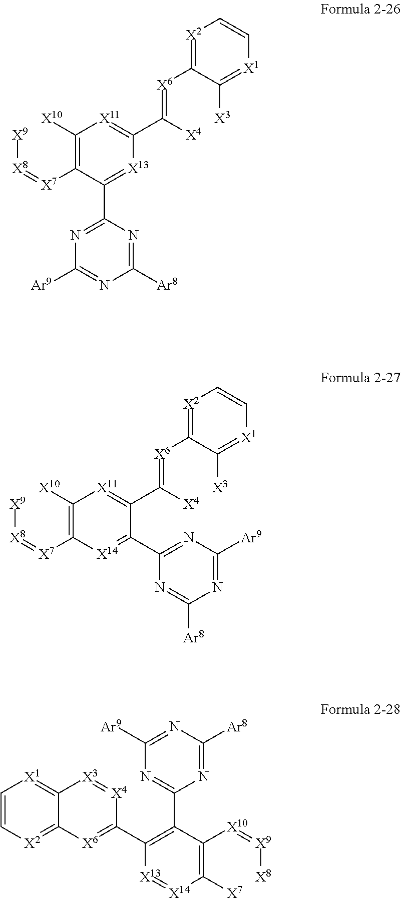
wherein X¹-X¹⁴ and Ar⁸-Ar⁹ are defined the same as that in claim 1.

6. The organic electroluminescent compound according to claim 1, wherein the structure of
the compound M is as shown in any one of M-1 to M-388 below:

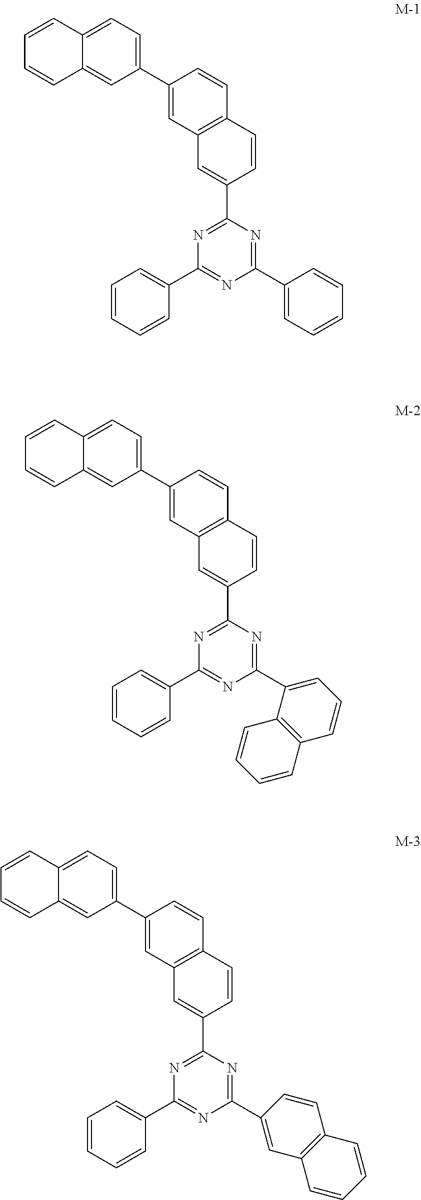 [SMILES: c1ccc(-c2nc(-c3ccccc3)nc(-c3ccc4ccc(-c5ccc6ccccc6c5)cc4c3)n2)cc1 ; c1ccc(-c2nc(-c3ccc4ccc(-c5ccc6ccccc6c5)cc4c3)nc(-c3cccc4ccccc34)n2)cc1 ; c1ccc(-c2nc(-c3ccc4ccccc4c3)nc(-c3ccc4ccc(-c5ccc6ccccc6c5)cc4c3)n2)cc1]

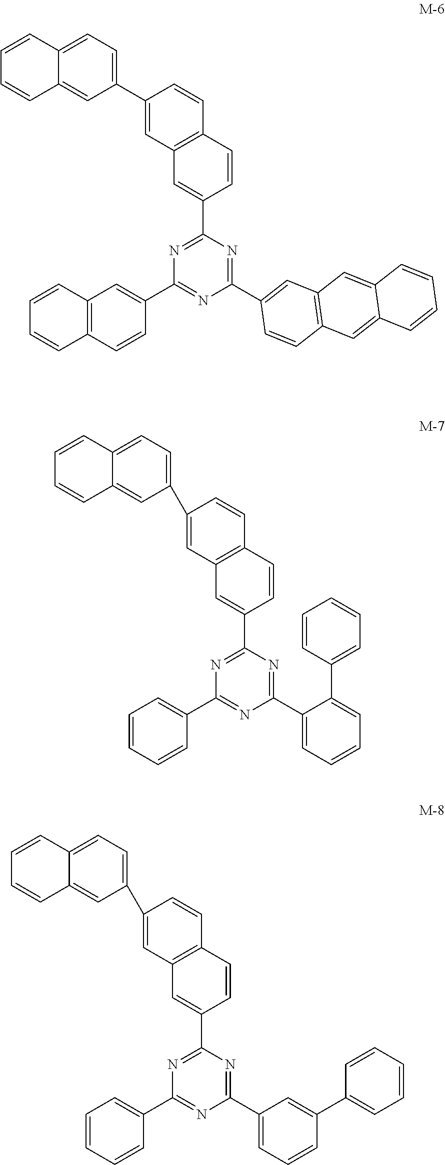 [SMILES: c1ccc2cc(-c3ccc4ccc(-c5nc(-c6ccc7ccccc7c6)nc(-c6ccc7cc8ccccc8cc7c6)n5)cc4c3)ccc2c1 ; c1ccc(-c2nc(-c3ccc4ccc(-c5ccc6ccccc6c5)cc4c3)nc(-c3ccccc3-c3ccccc3)n2)cc1 ; c1ccc(-c2cccc(-c3nc(-c4ccccc4)nc(-c4ccc5ccc(-c6ccc7ccccc7c6)cc5c4)n3)c2)cc1]

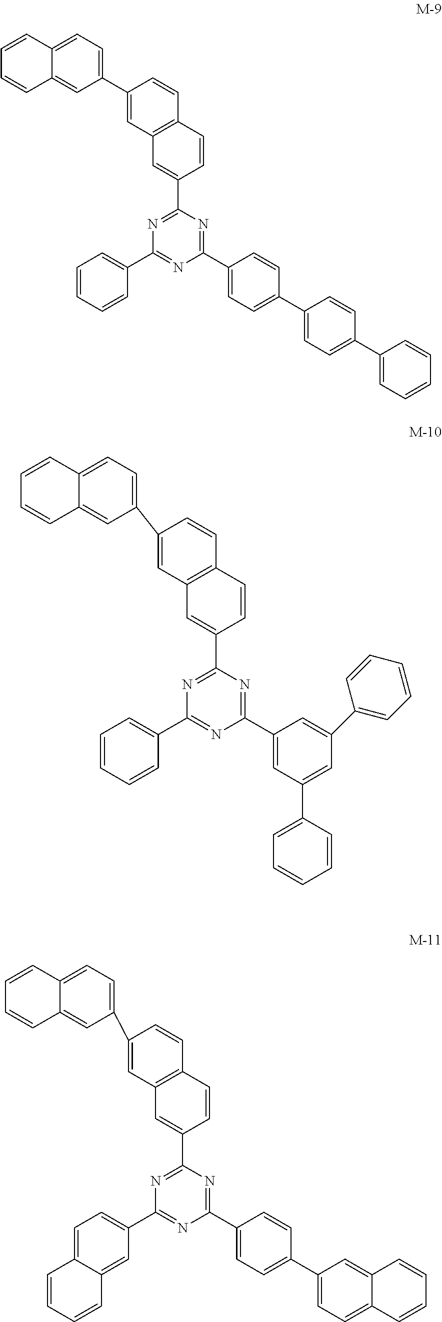 [SMILES: c1ccc(-c2ccc(-c3ccc(-c4nc(-c5ccccc5)nc(-c5ccc6ccc(-c7ccc8ccccc8c7)cc6c5)n4)cc3)cc2)cc1 ; c1ccc(-c2cc(-c3ccccc3)cc(-c3nc(-c4ccccc4)nc(-c4ccc5ccc(-c6ccc7ccccc7c6)cc5c4)n3)c2)cc1 ; c1ccc2cc(-c3ccc(-c4nc(-c5ccc6ccccc6c5)nc(-c5ccc6ccc(-c7ccc8ccccc8c7)cc6c5)n4)cc3)ccc2c1]

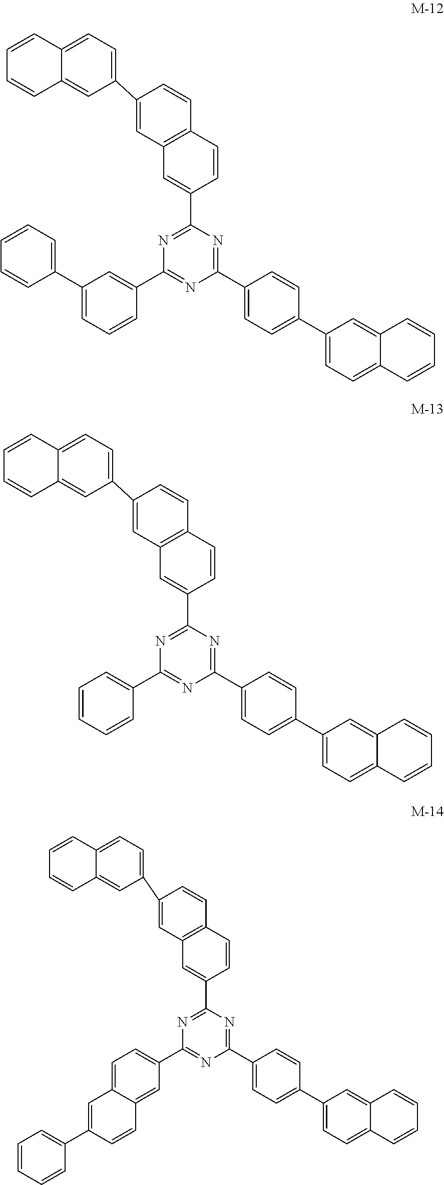 [SMILES: c1ccc(-c2cccc(-c3nc(-c4ccc(-c5ccc6ccccc6c5)cc4)nc(-c4ccc5ccc(-c6ccc7ccccc7c6)cc5c4)n3)c2)cc1 ; c1ccc(-c2nc(-c3ccc(-c4ccc5ccccc5c4)cc3)nc(-c3ccc4ccc(-c5ccc6ccccc6c5)cc4c3)n2)cc1 ; c1ccc(-c2ccc3cc(-c4nc(-c5ccc(-c6ccc7ccccc7c6)cc5)nc(-c5ccc6ccc(-c7ccc8ccccc8c7)cc6c5)n4)ccc3c2)cc1]

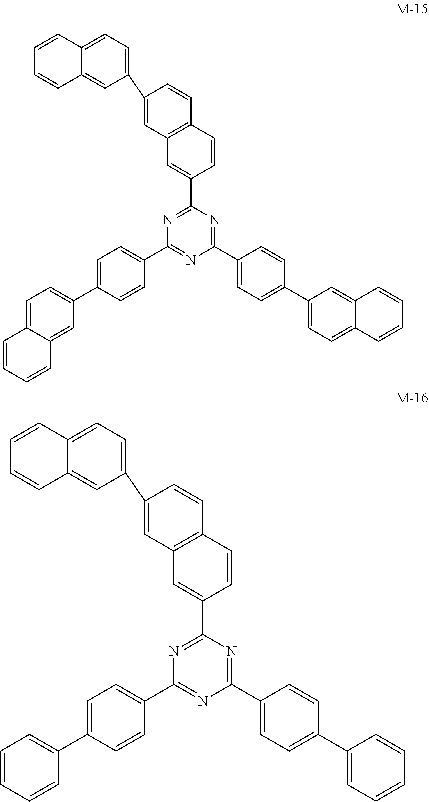 [SMILES: c1ccc2cc(-c3ccc(-c4nc(-c5ccc(-c6ccc7ccccc7c6)cc5)nc(-c5ccc6ccc(-c7ccc8ccccc8c7)cc6c5)n4)cc3)ccc2c1 ; c1ccc(-c2ccc(-c3nc(-c4ccc(-c5ccccc5)cc4)nc(-c4ccc5ccc(-c6ccc7ccccc7c6)cc5c4)n3)cc2)cc1]

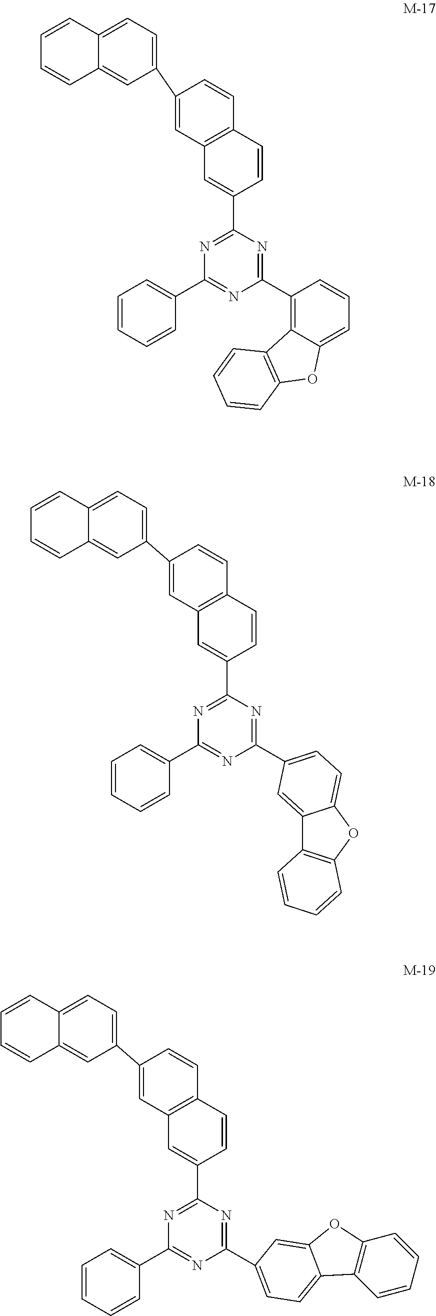 [SMILES: c1ccc(-c2nc(-c3ccc4ccc(-c5ccc6ccccc6c5)cc4c3)nc(-c3cccc4oc5ccccc5c34)n2)cc1 ; c1ccc(-c2nc(-c3ccc4ccc(-c5ccc6ccccc6c5)cc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4c(c3)oc3ccccc34)n2)cc1]

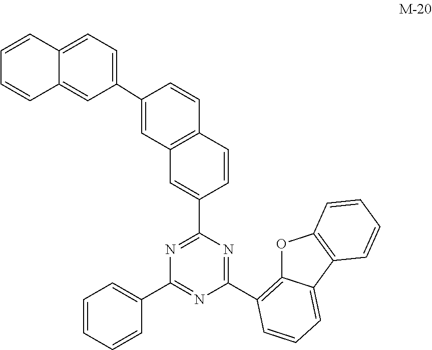 [SMILES: c1ccc(-c2nc(-c3ccc4ccc(-c5ccc6ccccc6c5)cc4c3)nc(-c3cccc4c3oc3ccccc34)n2)cc1]

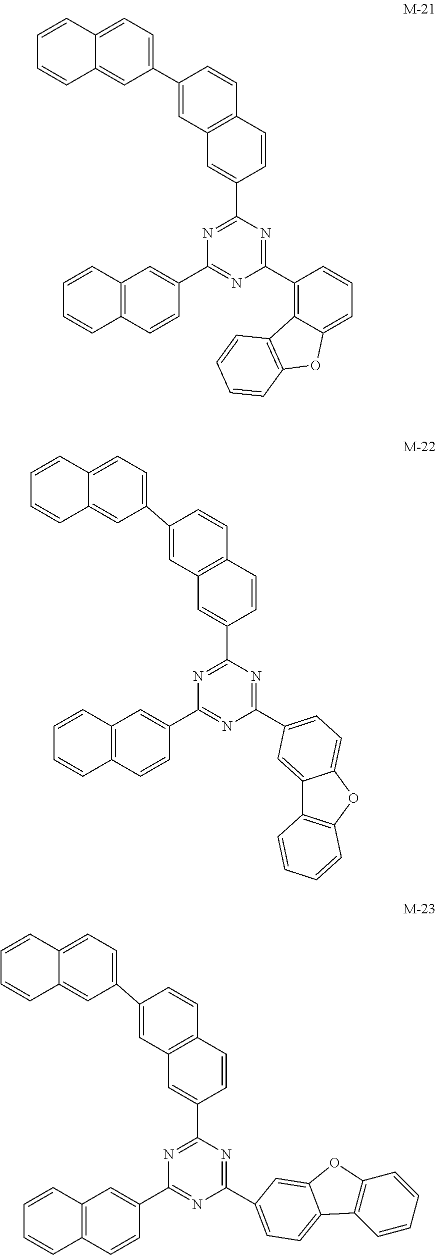 [SMILES: c1ccc2cc(-c3ccc4ccc(-c5nc(-c6ccc7ccccc7c6)nc(-c6cccc7oc8ccccc8c67)n5)cc4c3)ccc2c1 ; c1ccc2cc(-c3ccc4ccc(-c5nc(-c6ccc7ccccc7c6)nc(-c6ccc7oc8ccccc8c7c6)n5)cc4c3)ccc2c1 ; c1ccc2cc(-c3ccc4ccc(-c5nc(-c6ccc7ccccc7c6)nc(-c6ccc7c(c6)oc6ccccc67)n5)cc4c3)ccc2c1]

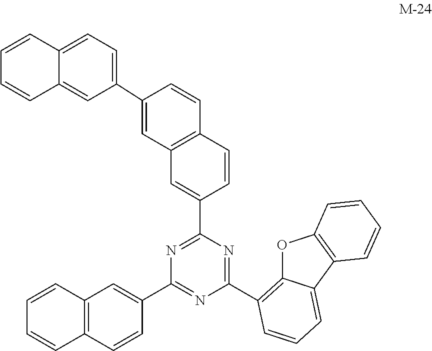 [SMILES: c1ccc2cc(-c3ccc4ccc(-c5nc(-c6ccc7ccccc7c6)nc(-c6cccc7c6oc6ccccc67)n5)cc4c3)ccc2c1]

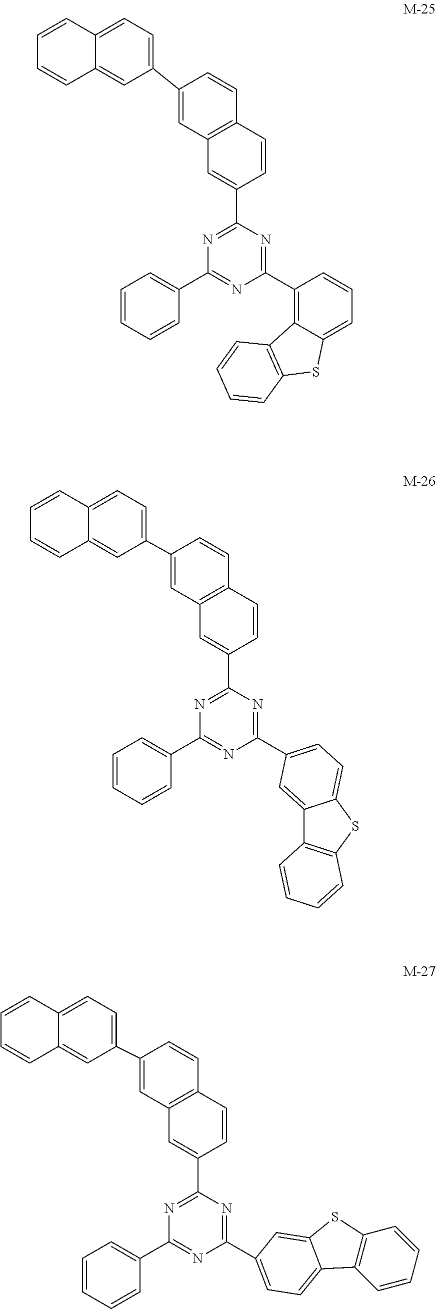 [SMILES: c1ccc(-c2nc(-c3ccc4ccc(-c5ccc6ccccc6c5)cc4c3)nc(-c3cccc4sc5ccccc5c34)n2)cc1 ; c1ccc(-c2nc(-c3ccc4ccc(-c5ccc6ccccc6c5)cc4c3)nc(-c3ccc4sc5ccccc5c4c3)n2)cc1 ; c1ccc(-c2nc(-c3ccc4ccc(-c5ccc6ccccc6c5)cc4c3)nc(-c3ccc4c(c3)sc3ccccc34)n2)cc1]

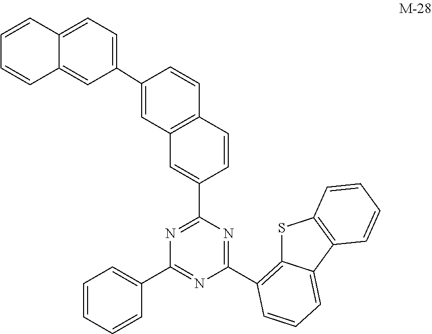 [SMILES: c1ccc(-c2nc(-c3ccc4ccc(-c5ccc6ccccc6c5)cc4c3)nc(-c3cccc4c3sc3ccccc34)n2)cc1]

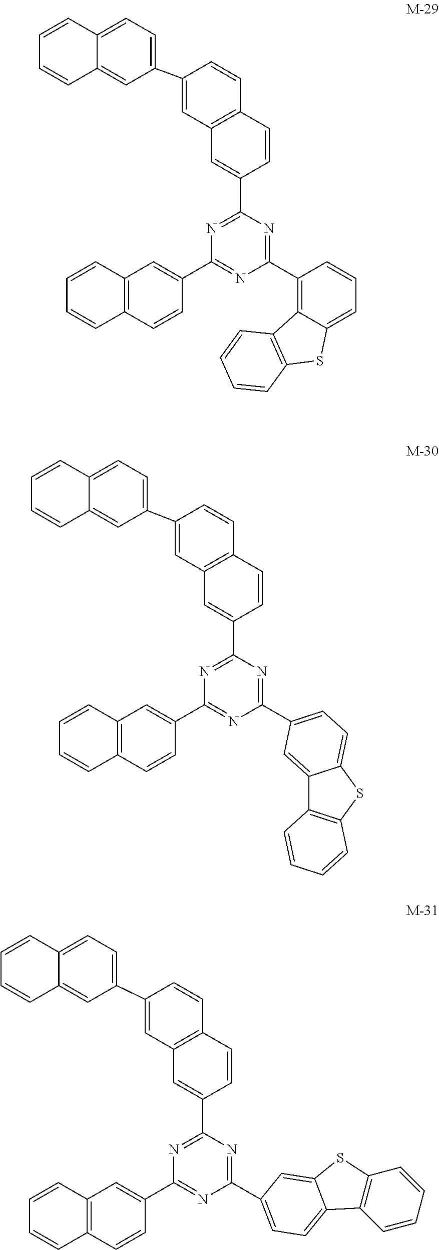 [SMILES: c1ccc2cc(-c3ccc4ccc(-c5nc(-c6ccc7ccccc7c6)nc(-c6cccc7sc8ccccc8c67)n5)cc4c3)ccc2c1 ; c1ccc2cc(-c3ccc4ccc(-c5nc(-c6ccc7ccccc7c6)nc(-c6ccc7sc8ccccc8c7c6)n5)cc4c3)ccc2c1 ; c1ccc2cc(-c3ccc4ccc(-c5nc(-c6ccc7ccccc7c6)nc(-c6ccc7c(c6)sc6ccccc67)n5)cc4c3)ccc2c1]

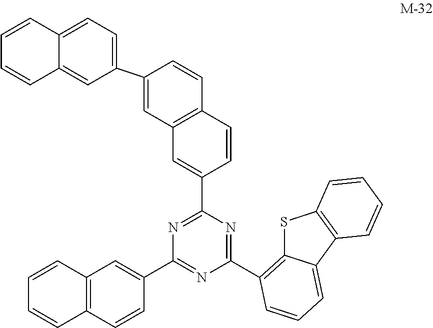 [SMILES: c1ccc2cc(-c3ccc4ccc(-c5nc(-c6ccc7ccccc7c6)nc(-c6cccc7c6sc6ccccc67)n5)cc4c3)ccc2c1]

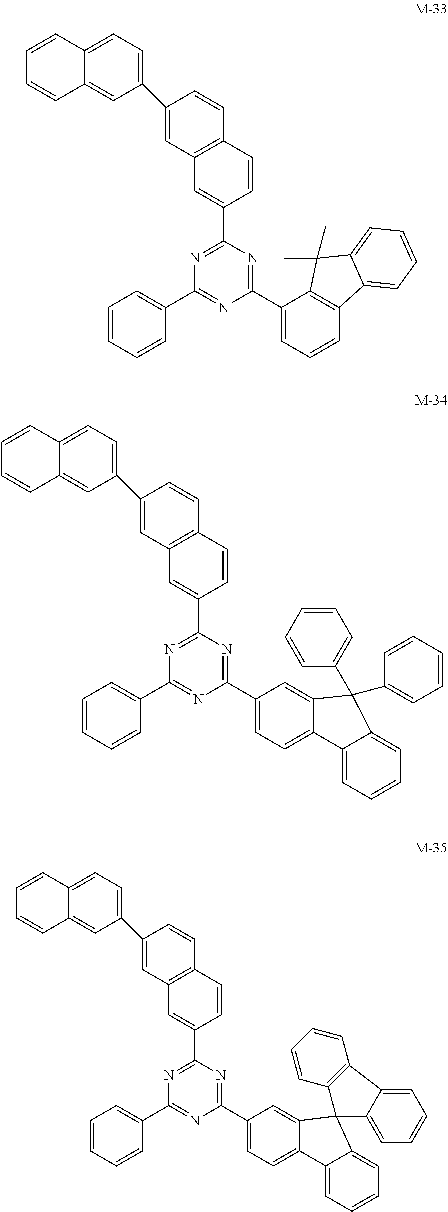 [SMILES: CC1(C)c2ccccc2-c2cccc(-c3nc(-c4ccccc4)nc(-c4ccc5ccc(-c6ccc7ccccc7c6)cc5c4)n3)c21 ; c1ccc(-c2nc(-c3ccc4c(c3)C(c3ccccc3)(c3ccccc3)c3ccccc3-4)nc(-c3ccc4ccc(-c5ccc6ccccc6c5)cc4c3)n2)cc1 ; c1ccc(-c2nc(-c3ccc4c(c3)C3(c5ccccc5-c5ccccc53)c3ccccc3-4)nc(-c3ccc4ccc(-c5ccc6ccccc6c5)cc4c3)n2)cc1]

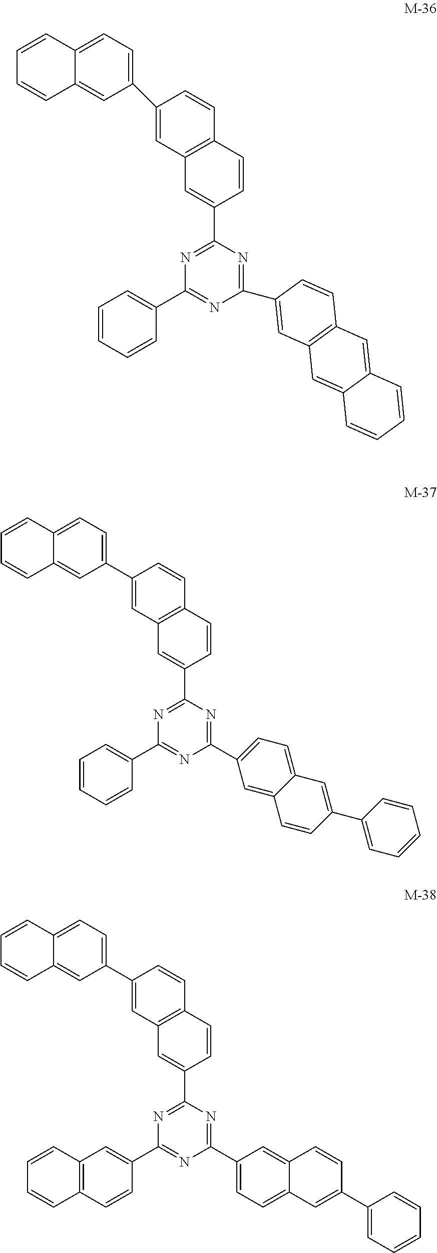 [SMILES: c1ccc(-c2nc(-c3ccc4ccc(-c5ccc6ccccc6c5)cc4c3)nc(-c3ccc4cc5ccccc5cc4c3)n2)cc1 ; c1ccc(-c2ccc3cc(-c4nc(-c5ccccc5)nc(-c5ccc6ccc(-c7ccc8ccccc8c7)cc6c5)n4)ccc3c2)cc1 ; c1ccc(-c2ccc3cc(-c4nc(-c5ccc6ccccc6c5)nc(-c5ccc6ccc(-c7ccc8ccccc8c7)cc6c5)n4)ccc3c2)cc1]

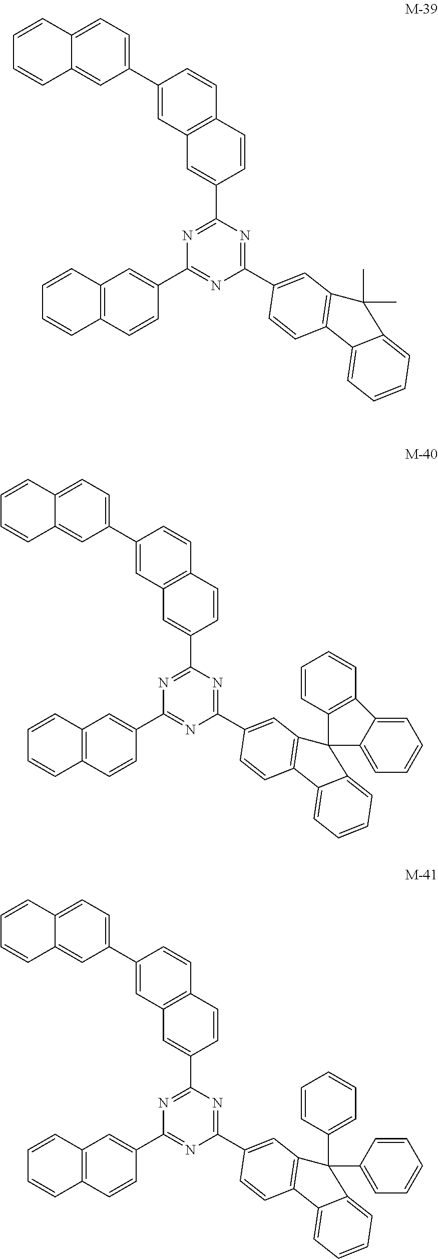 [SMILES: CC1(C)c2ccccc2-c2ccc(-c3nc(-c4ccc5ccccc5c4)nc(-c4ccc5ccc(-c6ccc7ccccc7c6)cc5c4)n3)cc21 ; c1ccc2c(c1)-c1ccccc1C21c2ccccc2-c2ccc(-c3nc(-c4ccc5ccccc5c4)nc(-c4ccc5ccc(-c6ccc7ccccc7c6)cc5c4)n3)cc21 ; c1ccc(C2(c3ccccc3)c3ccccc3-c3ccc(-c4nc(-c5ccc6ccccc6c5)nc(-c5ccc6ccc(-c7ccc8ccccc8c7)cc6c5)n4)cc32)cc1]

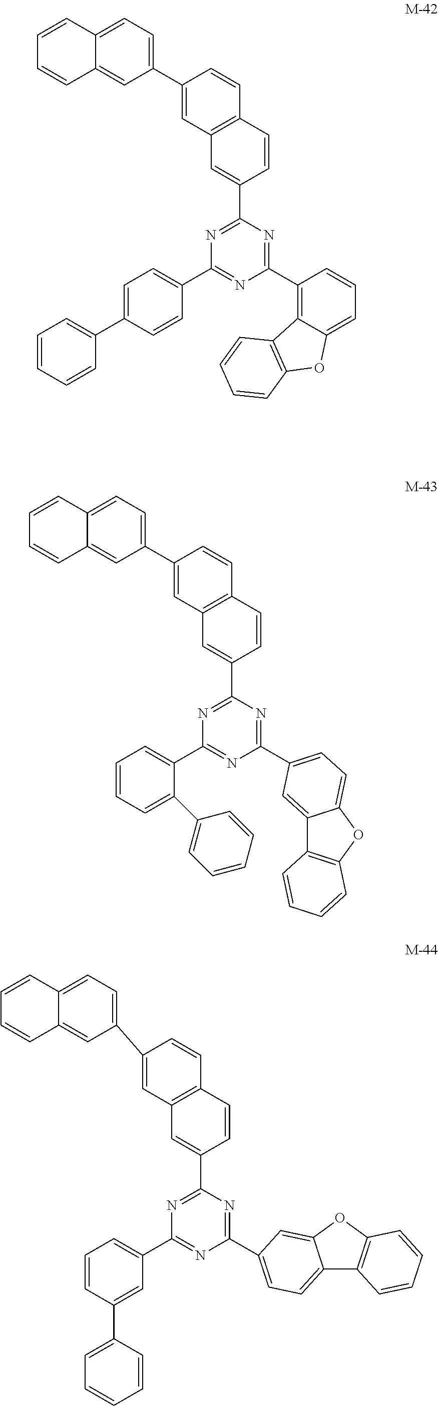 [SMILES: c1ccc(-c2ccc(-c3nc(-c4ccc5ccc(-c6ccc7ccccc7c6)cc5c4)nc(-c4cccc5oc6ccccc6c45)n3)cc2)cc1 ; c1ccc(-c2ccccc2-c2nc(-c3ccc4ccc(-c5ccc6ccccc6c5)cc4c3)nc(-c3ccc4oc5ccccc5c4c3)n2)cc1 ; c1ccc(-c2cccc(-c3nc(-c4ccc5ccc(-c6ccc7ccccc7c6)cc5c4)nc(-c4ccc5c(c4)oc4ccccc45)n3)c2)cc1]

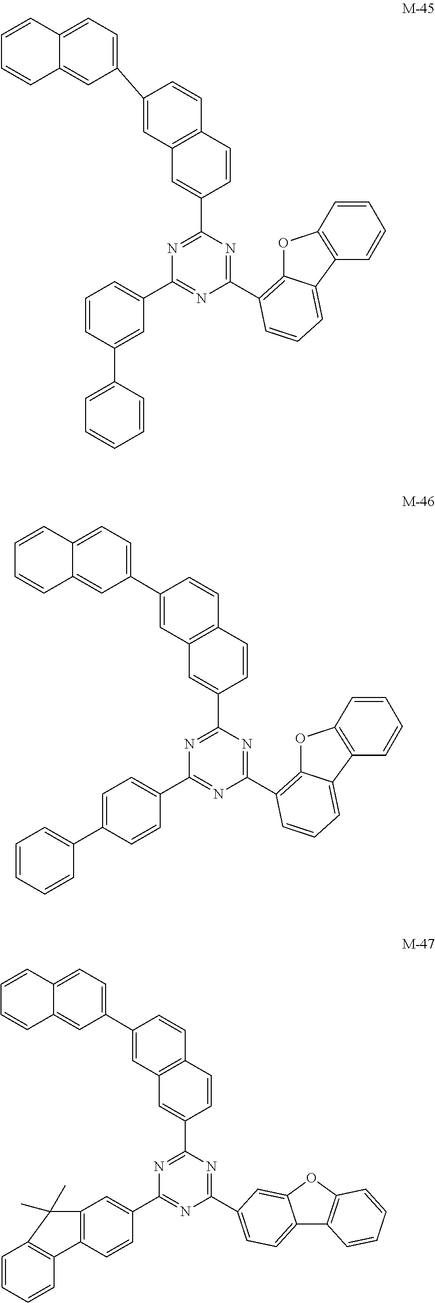 [SMILES: c1ccc(-c2cccc(-c3nc(-c4ccc5ccc(-c6ccc7ccccc7c6)cc5c4)nc(-c4cccc5c4oc4ccccc45)n3)c2)cc1 ; c1ccc(-c2ccc(-c3nc(-c4ccc5ccc(-c6ccc7ccccc7c6)cc5c4)nc(-c4cccc5c4oc4ccccc45)n3)cc2)cc1 ; CC1(C)c2ccccc2-c2ccc(-c3nc(-c4ccc5ccc(-c6ccc7ccccc7c6)cc5c4)nc(-c4ccc5c(c4)oc4ccccc45)n3)cc21]

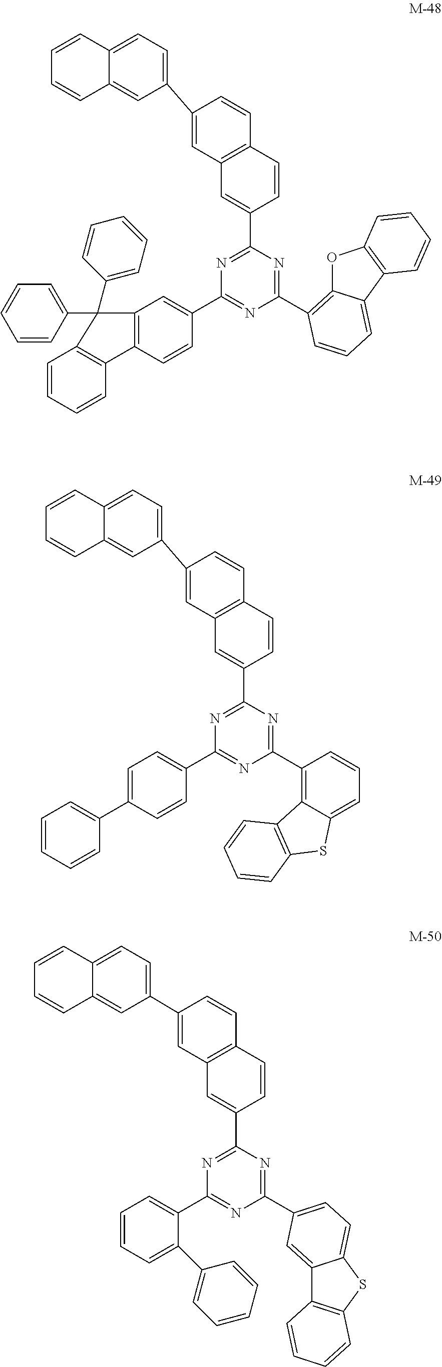 [SMILES: c1ccc(C2(c3ccccc3)c3ccccc3-c3ccc(-c4nc(-c5ccc6ccc(-c7ccc8ccccc8c7)cc6c5)nc(-c5cccc6c5oc5ccccc56)n4)cc32)cc1 ; c1ccc(-c2ccc(-c3nc(-c4ccc5ccc(-c6ccc7ccccc7c6)cc5c4)nc(-c4cccc5sc6ccccc6c45)n3)cc2)cc1 ; c1ccc(-c2ccccc2-c2nc(-c3ccc4ccc(-c5ccc6ccccc6c5)cc4c3)nc(-c3ccc4sc5ccccc5c4c3)n2)cc1]

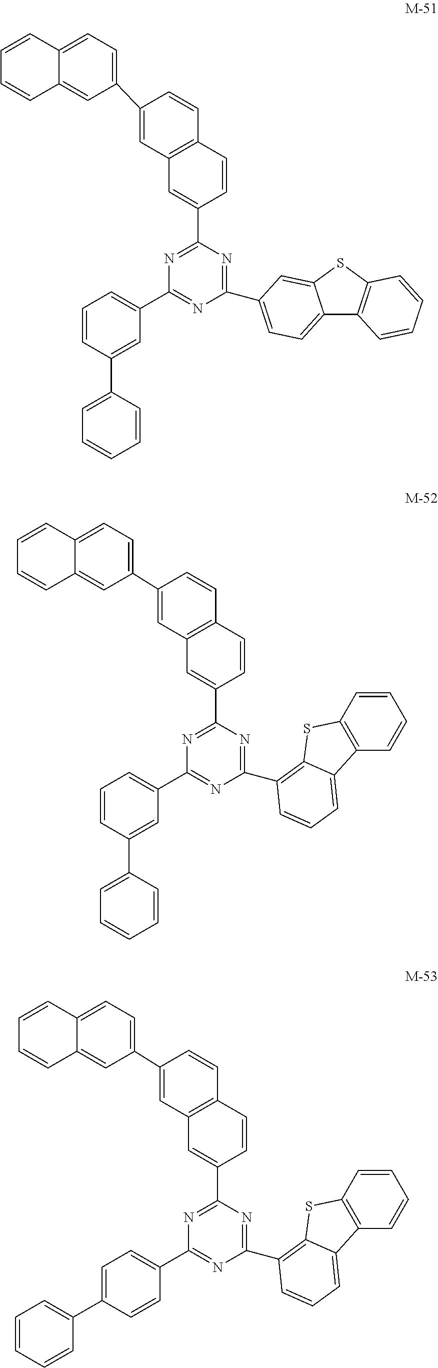 [SMILES: c1ccc(-c2cccc(-c3nc(-c4ccc5ccc(-c6ccc7ccccc7c6)cc5c4)nc(-c4ccc5c(c4)sc4ccccc45)n3)c2)cc1 ; c1ccc(-c2cccc(-c3nc(-c4ccc5ccc(-c6ccc7ccccc7c6)cc5c4)nc(-c4cccc5c4sc4ccccc45)n3)c2)cc1 ; c1ccc(-c2ccc(-c3nc(-c4ccc5ccc(-c6ccc7ccccc7c6)cc5c4)nc(-c4cccc5c4sc4ccccc45)n3)cc2)cc1]

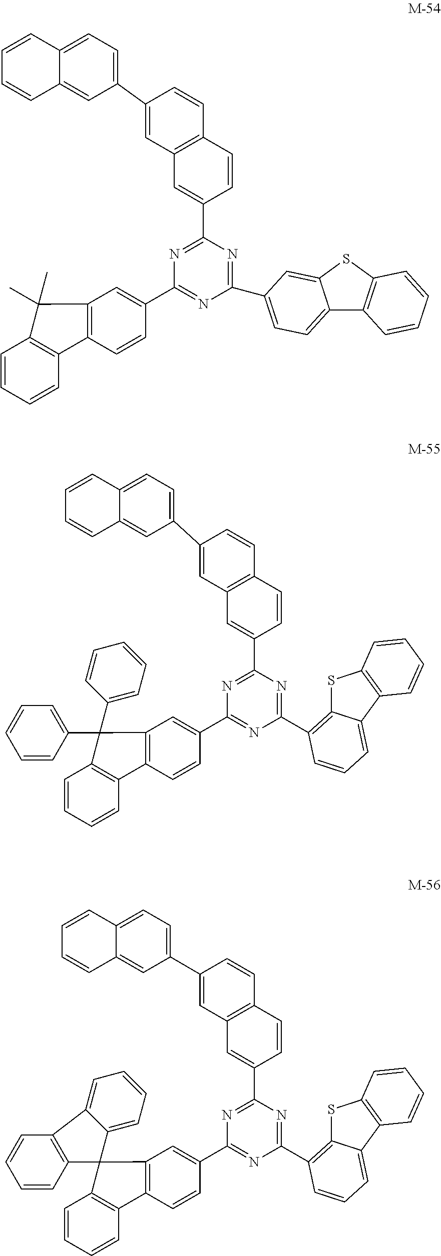 [SMILES: CC1(C)c2ccccc2-c2ccc(-c3nc(-c4ccc5ccc(-c6ccc7ccccc7c6)cc5c4)nc(-c4ccc5c(c4)sc4ccccc45)n3)cc21 ; c1ccc(C2(c3ccccc3)c3ccccc3-c3ccc(-c4nc(-c5ccc6ccc(-c7ccc8ccccc8c7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6ccc7ccccc7c6)cc5c4)nc(-c4cccc5c4sc4ccccc45)n3)cc21]

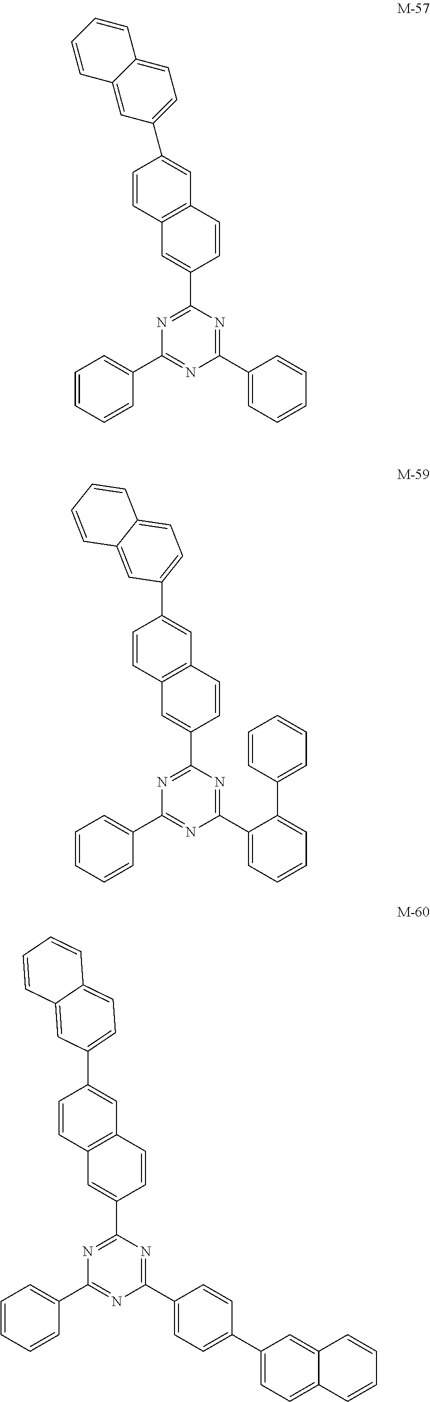 [SMILES: c1ccc(-c2nc(-c3ccccc3)nc(-c3ccc4cc(-c5ccc6ccccc6c5)ccc4c3)n2)cc1 ; c1ccc(-c2nc(-c3ccc4cc(-c5ccc6ccccc6c5)ccc4c3)nc(-c3ccccc3-c3ccccc3)n2)cc1 ; c1ccc(-c2nc(-c3ccc(-c4ccc5ccccc5c4)cc3)nc(-c3ccc4cc(-c5ccc6ccccc6c5)ccc4c3)n2)cc1]

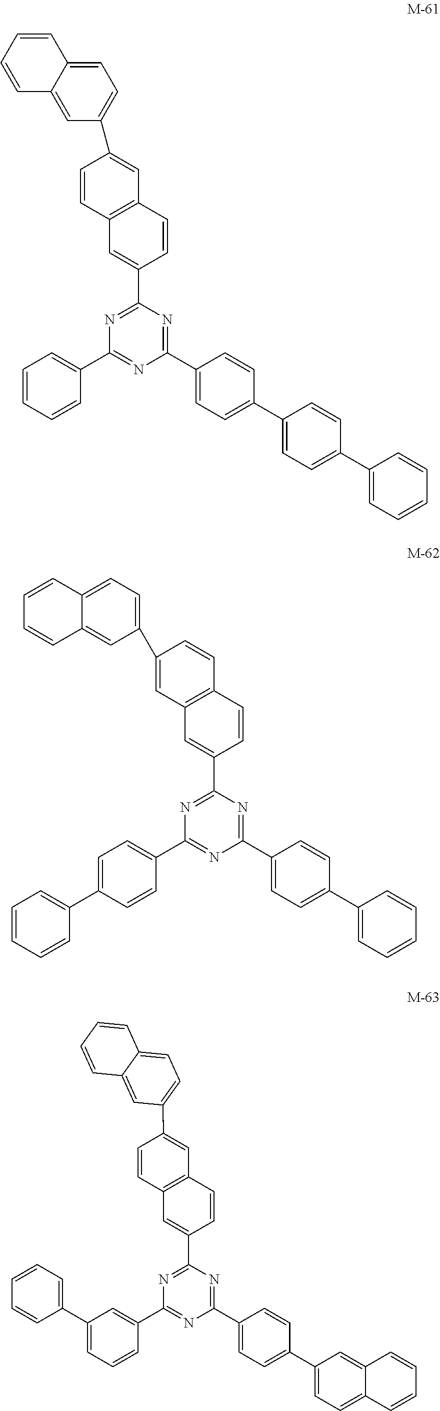 [SMILES: c1ccc(-c2ccc(-c3ccc(-c4nc(-c5ccccc5)nc(-c5ccc6cc(-c7ccc8ccccc8c7)ccc6c5)n4)cc3)cc2)cc1 ; c1ccc(-c2ccc(-c3nc(-c4ccc(-c5ccccc5)cc4)nc(-c4ccc5ccc(-c6ccc7ccccc7c6)cc5c4)n3)cc2)cc1 ; c1ccc(-c2cccc(-c3nc(-c4ccc(-c5ccc6ccccc6c5)cc4)nc(-c4ccc5cc(-c6ccc7ccccc7c6)ccc5c4)n3)c2)cc1]

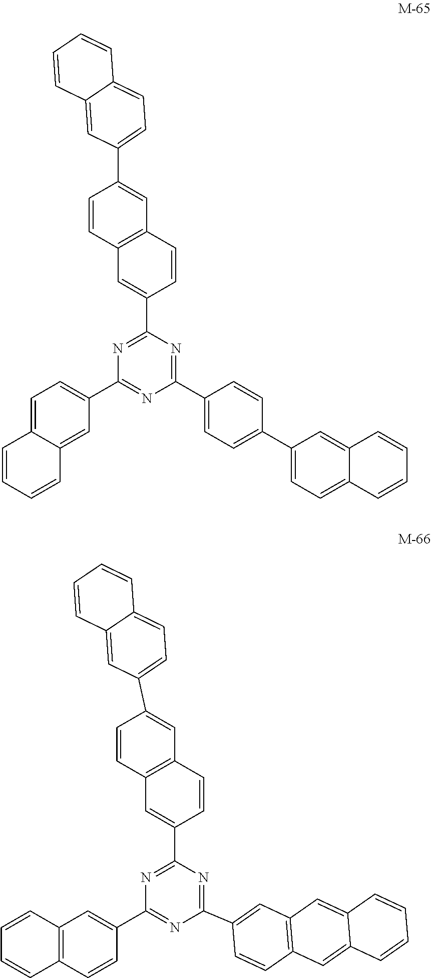 [SMILES: c1ccc2cc(-c3ccc(-c4nc(-c5ccc6ccccc6c5)nc(-c5ccc6cc(-c7ccc8ccccc8c7)ccc6c5)n4)cc3)ccc2c1 ; c1ccc2cc(-c3ccc4cc(-c5nc(-c6ccc7ccccc7c6)nc(-c6ccc7cc8ccccc8cc7c6)n5)ccc4c3)ccc2c1]

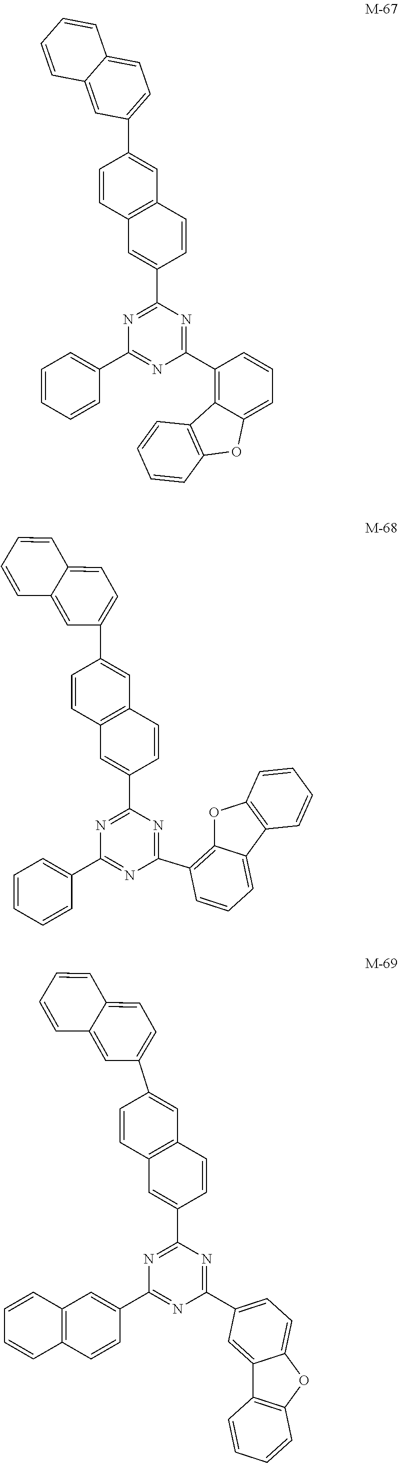 [SMILES: c1ccc(-c2nc(-c3ccc4cc(-c5ccc6ccccc6c5)ccc4c3)nc(-c3cccc4oc5ccccc5c34)n2)cc1 ; c1ccc(-c2nc(-c3ccc4cc(-c5ccc6ccccc6c5)ccc4c3)nc(-c3cccc4c3oc3ccccc34)n2)cc1 ; c1ccc2cc(-c3ccc4cc(-c5nc(-c6ccc7ccccc7c6)nc(-c6ccc7oc8ccccc8c7c6)n5)ccc4c3)ccc2c1]

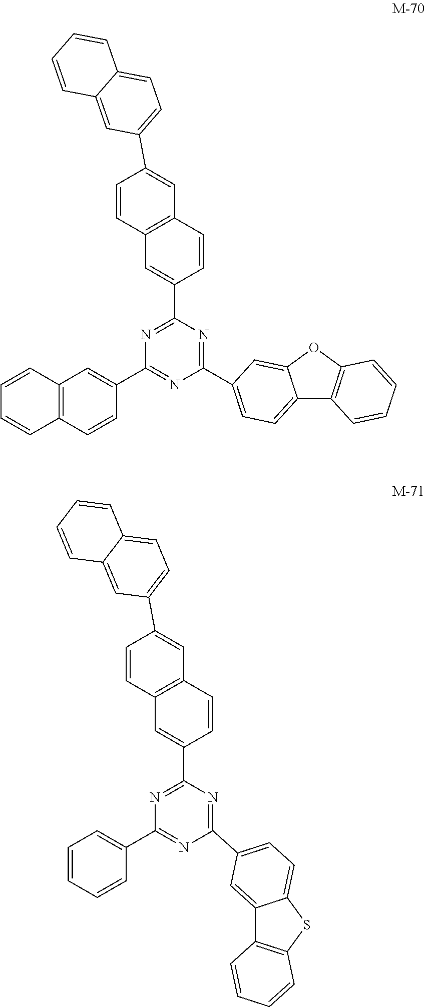 [SMILES: c1ccc2cc(-c3ccc4cc(-c5nc(-c6ccc7ccccc7c6)nc(-c6ccc7c(c6)oc6ccccc67)n5)ccc4c3)ccc2c1 ; c1ccc(-c2nc(-c3ccc4cc(-c5ccc6ccccc6c5)ccc4c3)nc(-c3ccc4sc5ccccc5c4c3)n2)cc1]

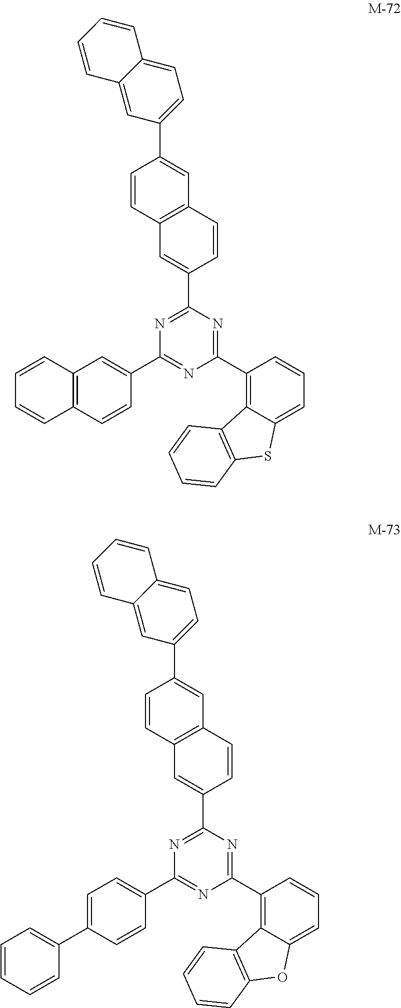 [SMILES: c1ccc2cc(-c3ccc4cc(-c5nc(-c6ccc7ccccc7c6)nc(-c6cccc7sc8ccccc8c67)n5)ccc4c3)ccc2c1 ; c1ccc(-c2ccc(-c3nc(-c4ccc5cc(-c6ccc7ccccc7c6)ccc5c4)nc(-c4cccc5oc6ccccc6c45)n3)cc2)cc1]

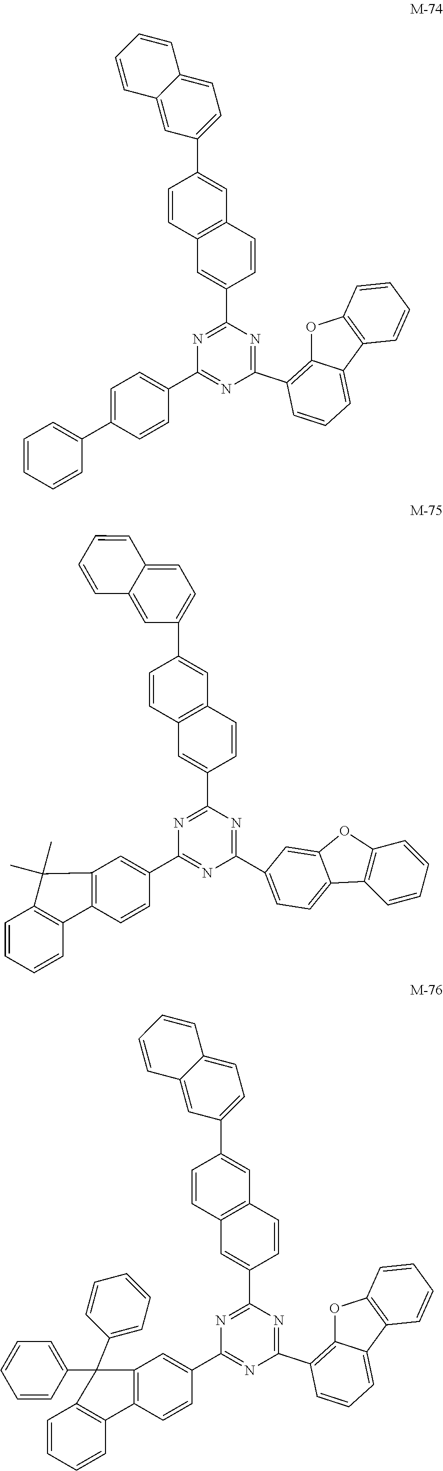 [SMILES: c1ccc(-c2ccc(-c3nc(-c4ccc5cc(-c6ccc7ccccc7c6)ccc5c4)nc(-c4cccc5c4oc4ccccc45)n3)cc2)cc1 ; CC1(C)c2ccccc2-c2ccc(-c3nc(-c4ccc5cc(-c6ccc7ccccc7c6)ccc5c4)nc(-c4ccc5c(c4)oc4ccccc45)n3)cc21 ; c1ccc(C2(c3ccccc3)c3ccccc3-c3ccc(-c4nc(-c5ccc6cc(-c7ccc8ccccc8c7)ccc6c5)nc(-c5cccc6c5oc5ccccc56)n4)cc32)cc1]

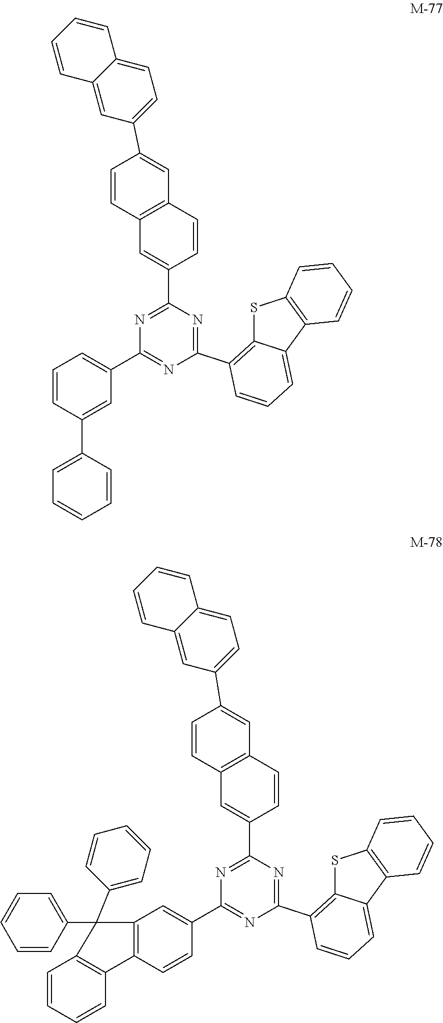 [SMILES: c1ccc(-c2cccc(-c3nc(-c4ccc5cc(-c6ccc7ccccc7c6)ccc5c4)nc(-c4cccc5c4sc4ccccc45)n3)c2)cc1 ; c1ccc(C2(c3ccccc3)c3ccccc3-c3ccc(-c4nc(-c5ccc6cc(-c7ccc8ccccc8c7)ccc6c5)nc(-c5cccc6c5sc5ccccc56)n4)cc32)cc1]

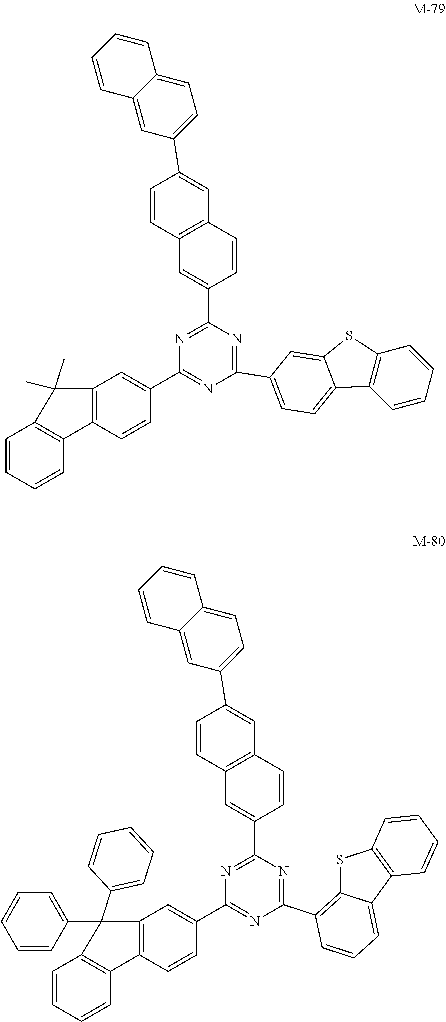 [SMILES: CC1(C)c2ccccc2-c2ccc(-c3nc(-c4ccc5cc(-c6ccc7ccccc7c6)ccc5c4)nc(-c4ccc5c(c4)sc4ccccc45)n3)cc21 ; c1ccc(C2(c3ccccc3)c3ccccc3-c3ccc(-c4nc(-c5ccc6cc(-c7ccc8ccccc8c7)ccc6c5)nc(-c5cccc6c5sc5ccccc56)n4)cc32)cc1]

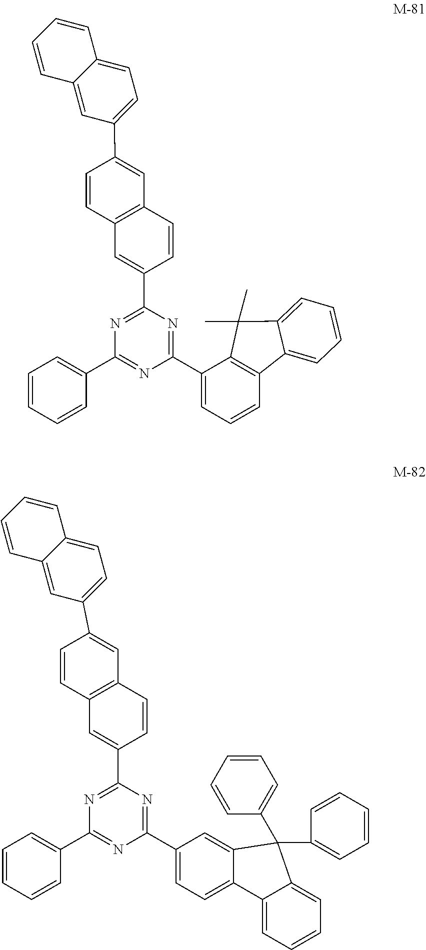 [SMILES: CC1(C)c2ccccc2-c2cccc(-c3nc(-c4ccccc4)nc(-c4ccc5cc(-c6ccc7ccccc7c6)ccc5c4)n3)c21 ; c1ccc(-c2nc(-c3ccc4c(c3)C(c3ccccc3)(c3ccccc3)c3ccccc3-4)nc(-c3ccc4cc(-c5ccc6ccccc6c5)ccc4c3)n2)cc1]

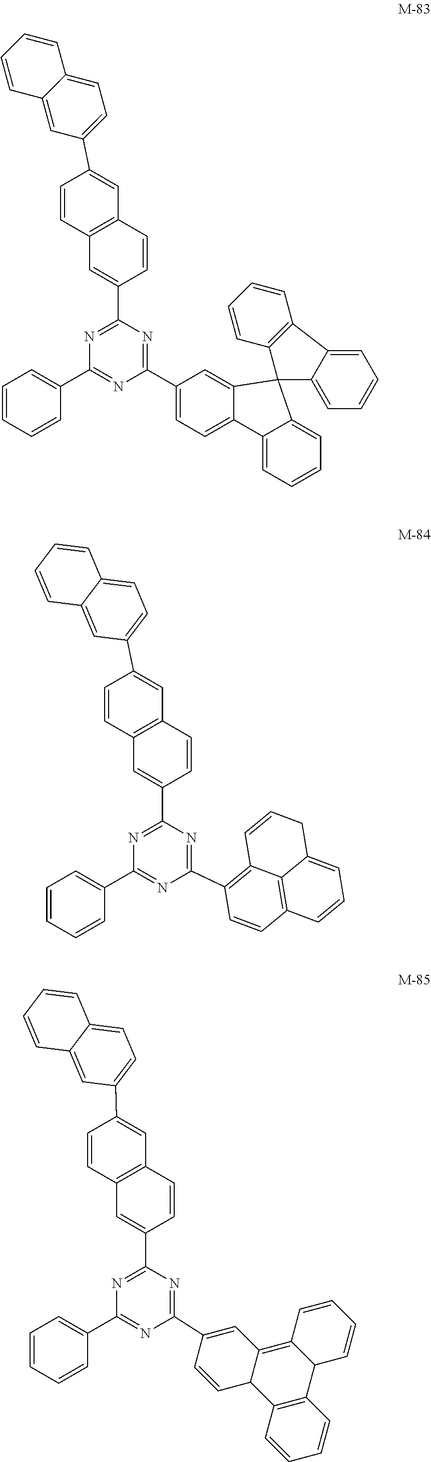 [SMILES: c1ccc(-c2nc(-c3ccc4c(c3)C3(c5ccccc5-c5ccccc53)c3ccccc3-4)nc(-c3ccc4cc(-c5ccc6ccccc6c5)ccc4c3)n2)cc1 ; C1=Cc2c(-c3nc(-c4ccccc4)nc(-c4ccc5cc(-c6ccc7ccccc7c6)ccc5c4)n3)ccc3cccc(c23)C1 ; C1=CC2=C3C=C(c4nc(-c5ccccc5)nc(-c5ccc6cc(-c7ccc8ccccc8c7)ccc6c5)n4)C=CC3c3ccccc3C2C=C1]

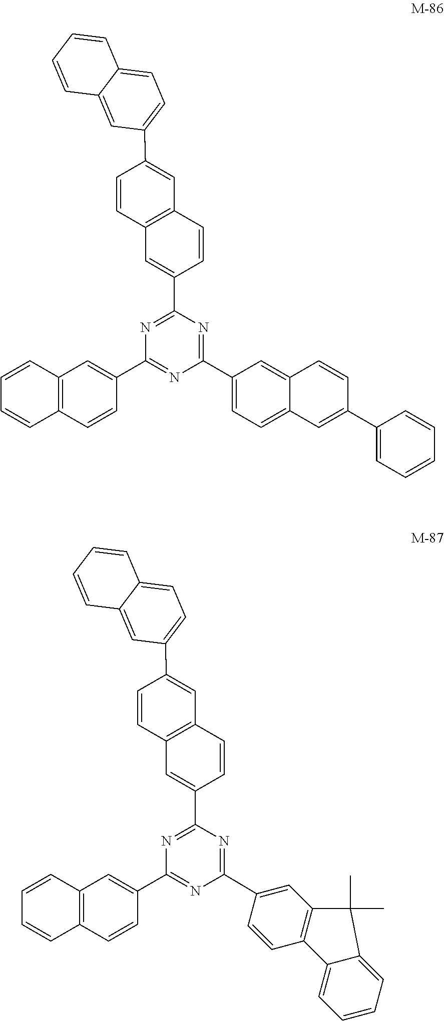 [SMILES: c1ccc(-c2ccc3cc(-c4nc(-c5ccc6ccccc6c5)nc(-c5ccc6cc(-c7ccc8ccccc8c7)ccc6c5)n4)ccc3c2)cc1 ; CC1(C)c2ccccc2-c2ccc(-c3nc(-c4ccc5ccccc5c4)nc(-c4ccc5cc(-c6ccc7ccccc7c6)ccc5c4)n3)cc21]

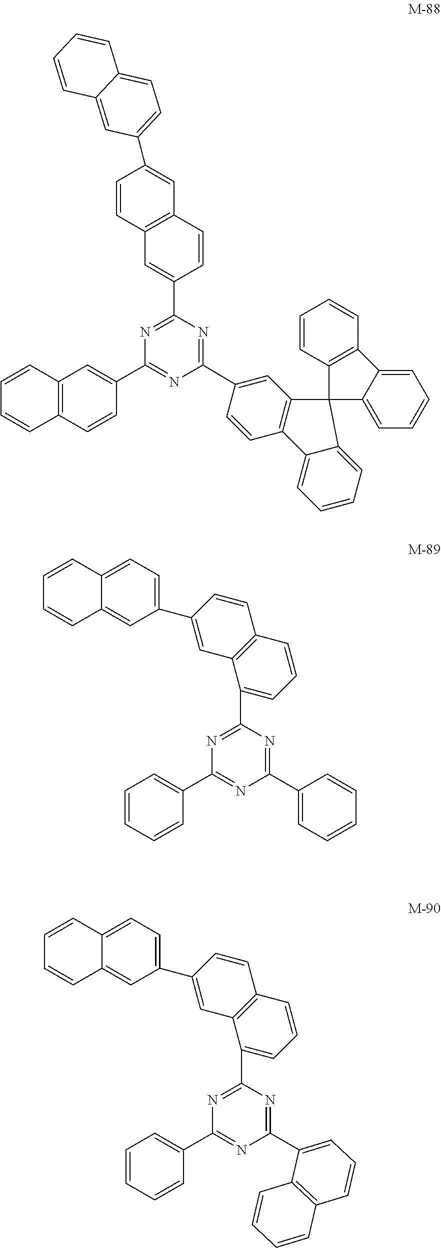 [SMILES: c1ccc2c(c1)-c1ccccc1C21c2ccccc2-c2ccc(-c3nc(-c4ccc5ccccc5c4)nc(-c4ccc5cc(-c6ccc7ccccc7c6)ccc5c4)n3)cc21 ; c1ccc(-c2nc(-c3ccccc3)nc(-c3cccc4ccc(-c5ccc6ccccc6c5)cc34)n2)cc1 ; c1ccc(-c2nc(-c3cccc4ccccc34)nc(-c3cccc4ccc(-c5ccc6ccccc6c5)cc34)n2)cc1]

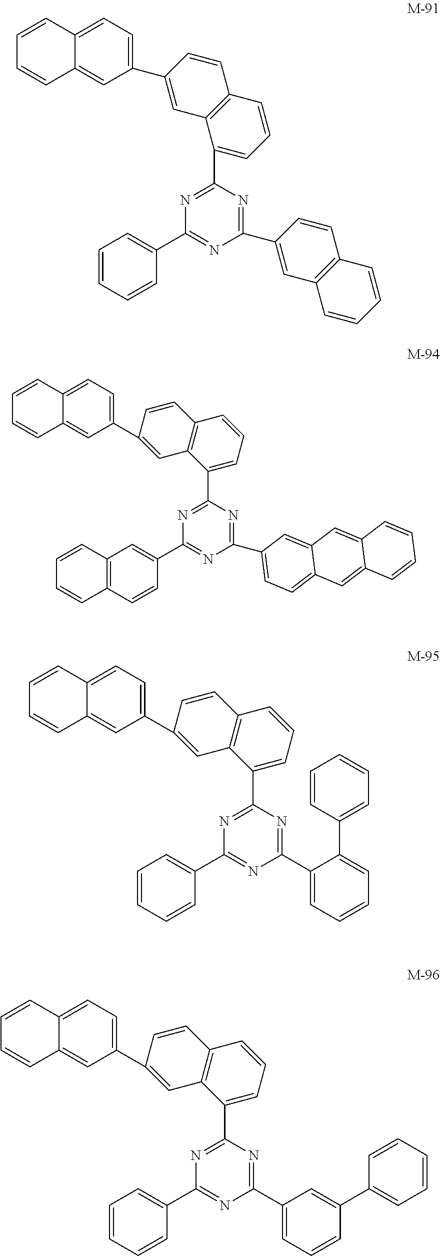 [SMILES: c1ccc(-c2nc(-c3ccc4ccccc4c3)nc(-c3cccc4ccc(-c5ccc6ccccc6c5)cc34)n2)cc1 ; c1ccc2cc(-c3ccc4cccc(-c5nc(-c6ccc7ccccc7c6)nc(-c6ccc7cc8ccccc8cc7c6)n5)c4c3)ccc2c1 ; c1ccc(-c2nc(-c3ccccc3-c3ccccc3)nc(-c3cccc4ccc(-c5ccc6ccccc6c5)cc34)n2)cc1 ; c1ccc(-c2cccc(-c3nc(-c4ccccc4)nc(-c4cccc5ccc(-c6ccc7ccccc7c6)cc45)n3)c2)cc1]

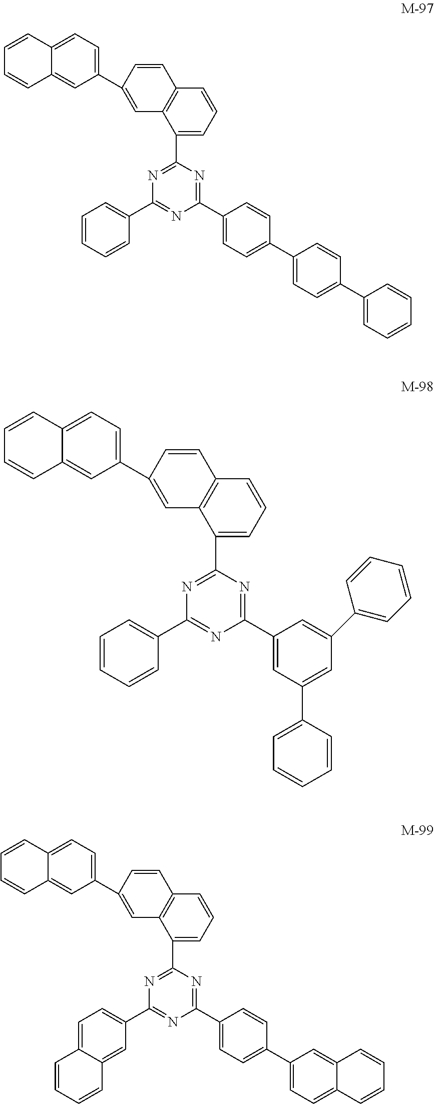 [SMILES: c1ccc(-c2ccc(-c3ccc(-c4nc(-c5ccccc5)nc(-c5cccc6ccc(-c7ccc8ccccc8c7)cc56)n4)cc3)cc2)cc1 ; c1ccc(-c2cc(-c3ccccc3)cc(-c3nc(-c4ccccc4)nc(-c4cccc5ccc(-c6ccc7ccccc7c6)cc45)n3)c2)cc1 ; c1ccc2cc(-c3ccc(-c4nc(-c5ccc6ccccc6c5)nc(-c5cccc6ccc(-c7ccc8ccccc8c7)cc56)n4)cc3)ccc2c1]

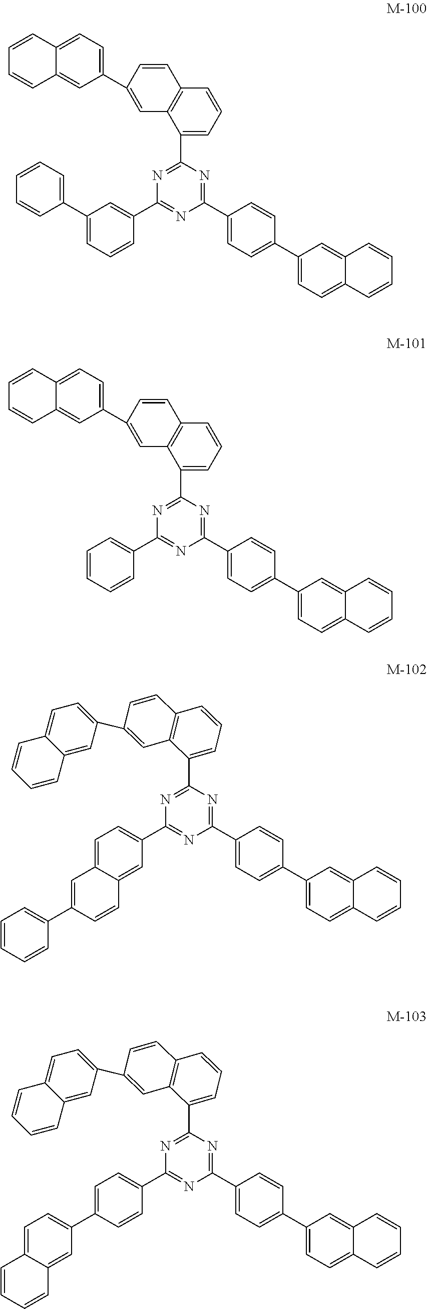 [SMILES: c1ccc(-c2cccc(-c3nc(-c4ccc(-c5ccc6ccccc6c5)cc4)nc(-c4cccc5ccc(-c6ccc7ccccc7c6)cc45)n3)c2)cc1 ; c1ccc(-c2nc(-c3ccc(-c4ccc5ccccc5c4)cc3)nc(-c3cccc4ccc(-c5ccc6ccccc6c5)cc34)n2)cc1 ; c1ccc(-c2ccc3cc(-c4nc(-c5ccc(-c6ccc7ccccc7c6)cc5)nc(-c5cccc6ccc(-c7ccc8ccccc8c7)cc56)n4)ccc3c2)cc1 ; c1ccc2cc(-c3ccc(-c4nc(-c5ccc(-c6ccc7ccccc7c6)cc5)nc(-c5cccc6ccc(-c7ccc8ccccc8c7)cc56)n4)cc3)ccc2c1]

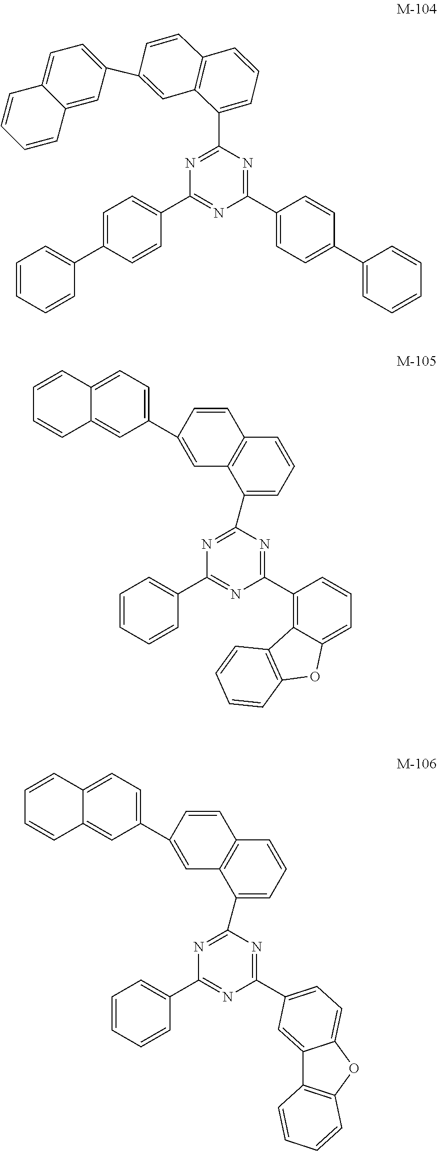 [SMILES: c1ccc(-c2ccc(-c3nc(-c4ccc(-c5ccccc5)cc4)nc(-c4cccc5ccc(-c6ccc7ccccc7c6)cc45)n3)cc2)cc1 ; c1ccc(-c2nc(-c3cccc4ccc(-c5ccc6ccccc6c5)cc34)nc(-c3cccc4oc5ccccc5c34)n2)cc1 ; c1ccc(-c2nc(-c3ccc4oc5ccccc5c4c3)nc(-c3cccc4ccc(-c5ccc6ccccc6c5)cc34)n2)cc1]

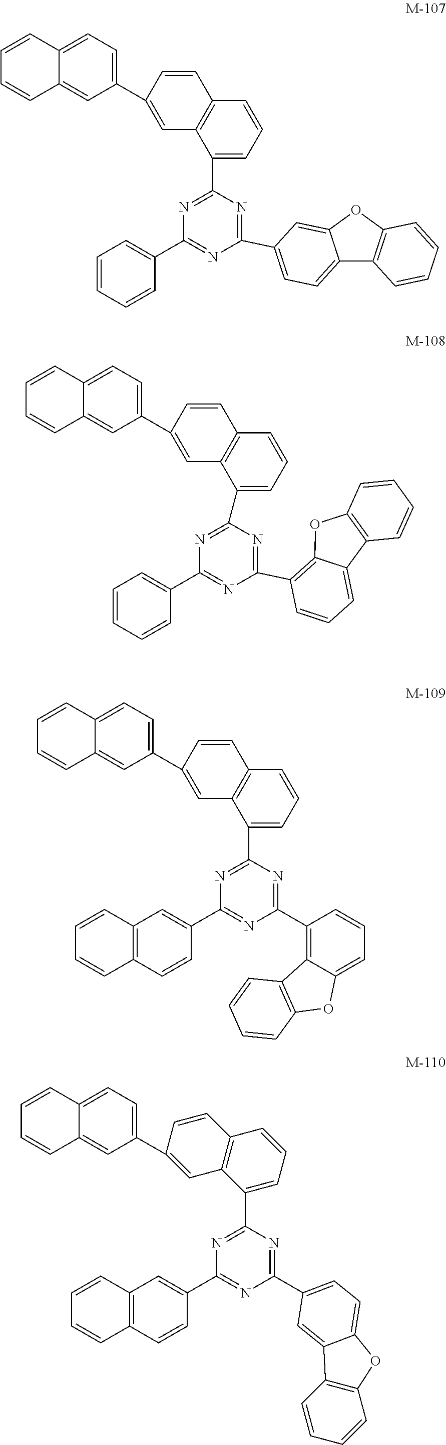 [SMILES: c1ccc(-c2nc(-c3ccc4c(c3)oc3ccccc34)nc(-c3cccc4ccc(-c5ccc6ccccc6c5)cc34)n2)cc1 ; c1ccc(-c2nc(-c3cccc4ccc(-c5ccc6ccccc6c5)cc34)nc(-c3cccc4c3oc3ccccc34)n2)cc1 ; c1ccc2cc(-c3ccc4cccc(-c5nc(-c6ccc7ccccc7c6)nc(-c6cccc7oc8ccccc8c67)n5)c4c3)ccc2c1 ; c1ccc2cc(-c3ccc4cccc(-c5nc(-c6ccc7ccccc7c6)nc(-c6ccc7oc8ccccc8c7c6)n5)c4c3)ccc2c1]

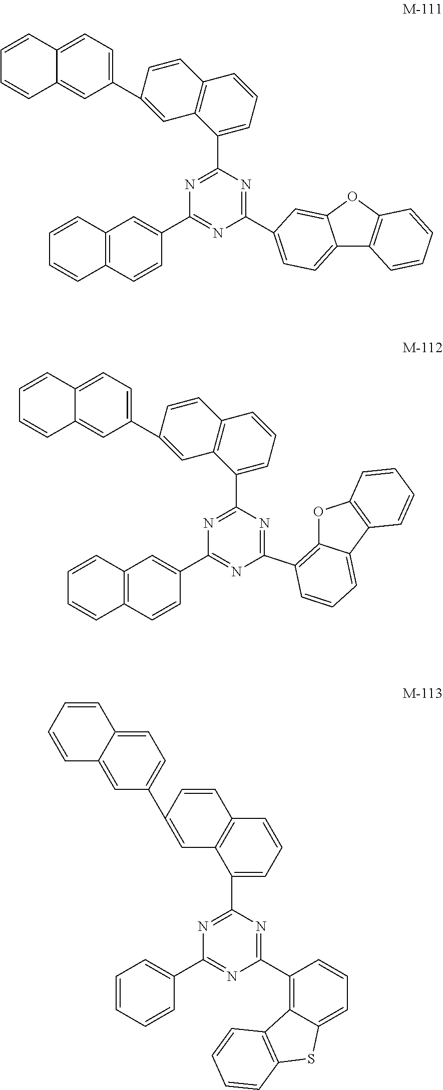 [SMILES: c1ccc2cc(-c3ccc4cccc(-c5nc(-c6ccc7ccccc7c6)nc(-c6ccc7c(c6)oc6ccccc67)n5)c4c3)ccc2c1 ; c1ccc2cc(-c3ccc4cccc(-c5nc(-c6ccc7ccccc7c6)nc(-c6cccc7c6oc6ccccc67)n5)c4c3)ccc2c1 ; c1ccc(-c2nc(-c3cccc4ccc(-c5ccc6ccccc6c5)cc34)nc(-c3cccc4sc5ccccc5c34)n2)cc1]

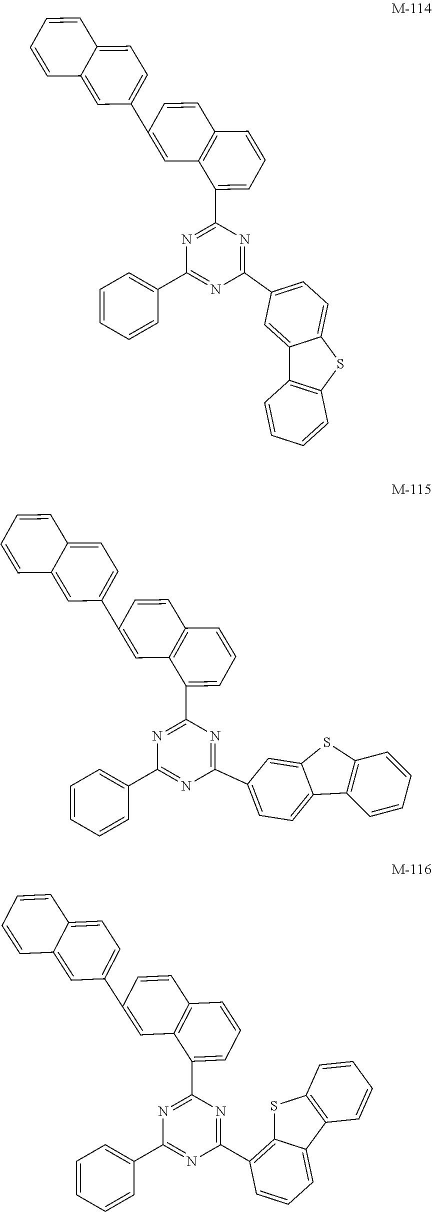 [SMILES: c1ccc(-c2nc(-c3ccc4sc5ccccc5c4c3)nc(-c3cccc4ccc(-c5ccc6ccccc6c5)cc34)n2)cc1 ; c1ccc(-c2nc(-c3ccc4c(c3)sc3ccccc34)nc(-c3cccc4ccc(-c5ccc6ccccc6c5)cc34)n2)cc1 ; c1ccc(-c2nc(-c3cccc4ccc(-c5ccc6ccccc6c5)cc34)nc(-c3cccc4c3sc3ccccc34)n2)cc1]

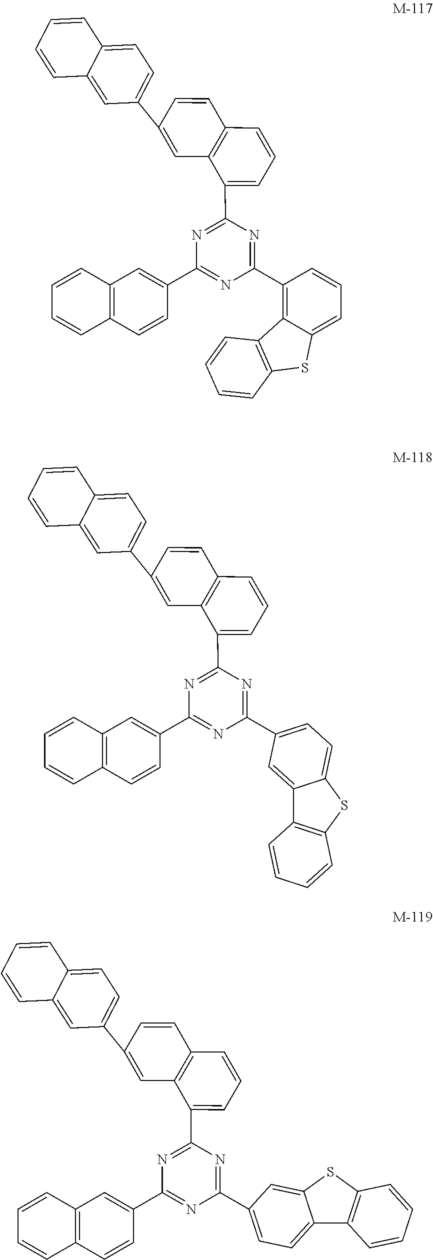 [SMILES: c1ccc2cc(-c3ccc4cccc(-c5nc(-c6ccc7ccccc7c6)nc(-c6cccc7sc8ccccc8c67)n5)c4c3)ccc2c1 ; c1ccc2cc(-c3ccc4cccc(-c5nc(-c6ccc7ccccc7c6)nc(-c6ccc7sc8ccccc8c7c6)n5)c4c3)ccc2c1 ; c1ccc2cc(-c3ccc4cccc(-c5nc(-c6ccc7ccccc7c6)nc(-c6ccc7c(c6)sc6ccccc67)n5)c4c3)ccc2c1]

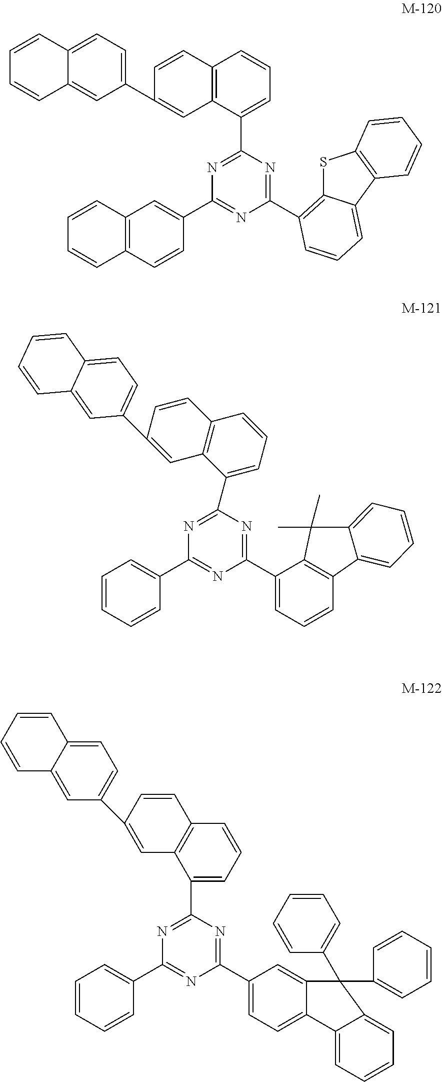 [SMILES: c1ccc2cc(-c3ccc4cccc(-c5nc(-c6ccc7ccccc7c6)nc(-c6cccc7c6sc6ccccc67)n5)c4c3)ccc2c1 ; CC1(C)c2ccccc2-c2cccc(-c3nc(-c4ccccc4)nc(-c4cccc5ccc(-c6ccc7ccccc7c6)cc45)n3)c21 ; c1ccc(-c2nc(-c3ccc4c(c3)C(c3ccccc3)(c3ccccc3)c3ccccc3-4)nc(-c3cccc4ccc(-c5ccc6ccccc6c5)cc34)n2)cc1]

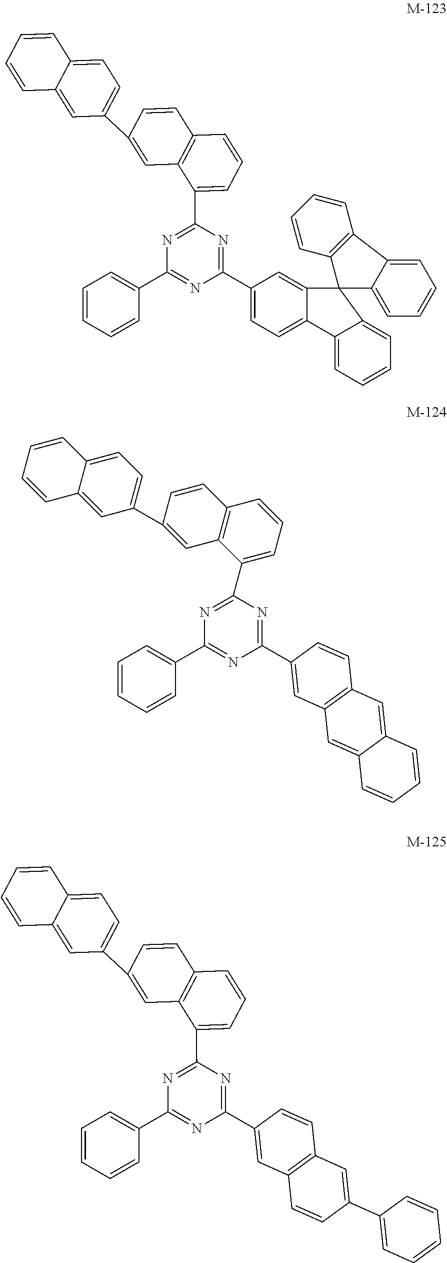 [SMILES: c1ccc(-c2nc(-c3ccc4c(c3)C3(c5ccccc5-c5ccccc53)c3ccccc3-4)nc(-c3cccc4ccc(-c5ccc6ccccc6c5)cc34)n2)cc1 ; c1ccc(-c2nc(-c3ccc4cc5ccccc5cc4c3)nc(-c3cccc4ccc(-c5ccc6ccccc6c5)cc34)n2)cc1 ; c1ccc(-c2ccc3cc(-c4nc(-c5ccccc5)nc(-c5cccc6ccc(-c7ccc8ccccc8c7)cc56)n4)ccc3c2)cc1]

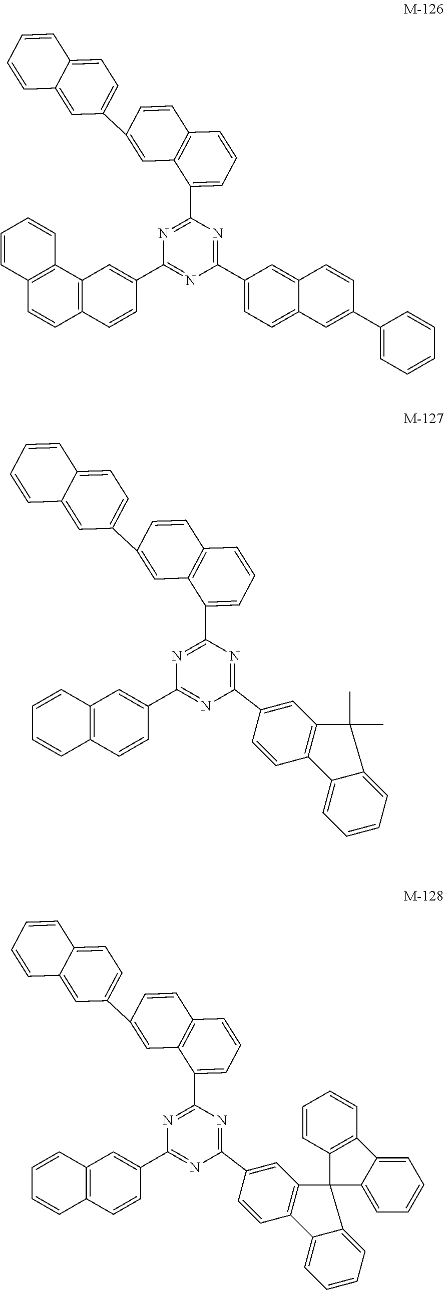 [SMILES: c1ccc(-c2ccc3cc(-c4nc(-c5ccc6ccc7ccccc7c6c5)nc(-c5cccc6ccc(-c7ccc8ccccc8c7)cc56)n4)ccc3c2)cc1 ; CC1(C)c2ccccc2-c2ccc(-c3nc(-c4ccc5ccccc5c4)nc(-c4cccc5ccc(-c6ccc7ccccc7c6)cc45)n3)cc21 ; c1ccc2c(c1)-c1ccccc1C21c2ccccc2-c2ccc(-c3nc(-c4ccc5ccccc5c4)nc(-c4cccc5ccc(-c6ccc7ccccc7c6)cc45)n3)cc21]

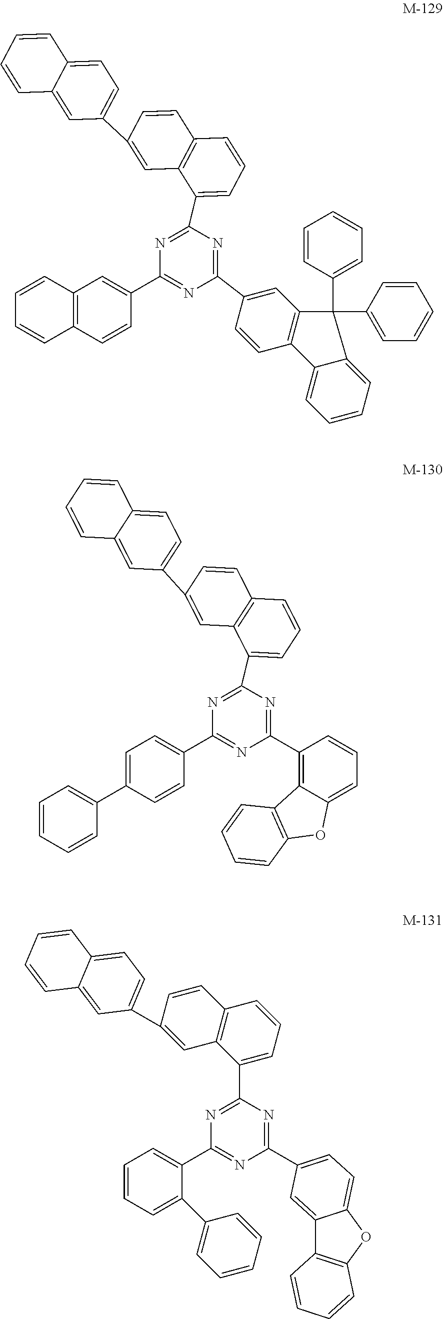 [SMILES: c1ccc(C2(c3ccccc3)c3ccccc3-c3ccc(-c4nc(-c5ccc6ccccc6c5)nc(-c5cccc6ccc(-c7ccc8ccccc8c7)cc56)n4)cc32)cc1 ; c1ccc(-c2ccc(-c3nc(-c4cccc5ccc(-c6ccc7ccccc7c6)cc45)nc(-c4cccc5oc6ccccc6c45)n3)cc2)cc1 ; c1ccc(-c2ccccc2-c2nc(-c3ccc4oc5ccccc5c4c3)nc(-c3cccc4ccc(-c5ccc6ccccc6c5)cc34)n2)cc1]

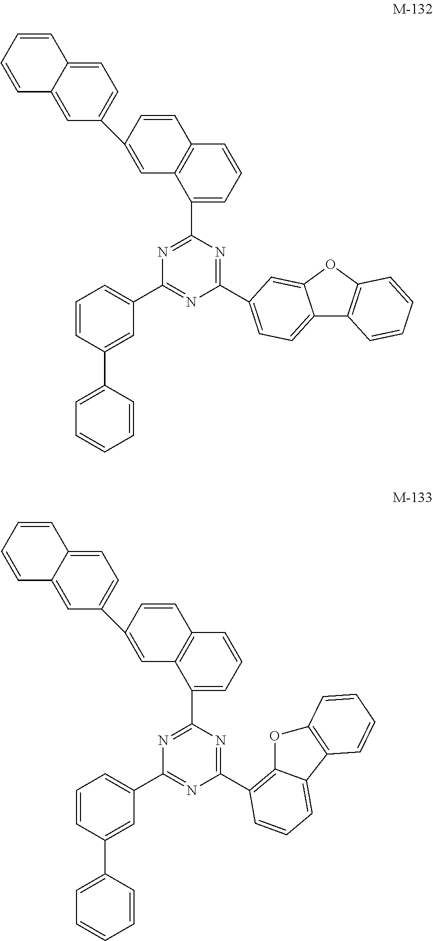 [SMILES: c1ccc(-c2cccc(-c3nc(-c4ccc5c(c4)oc4ccccc45)nc(-c4cccc5ccc(-c6ccc7ccccc7c6)cc45)n3)c2)cc1 ; c1ccc(-c2cccc(-c3nc(-c4cccc5ccc(-c6ccc7ccccc7c6)cc45)nc(-c4cccc5c4oc4ccccc45)n3)c2)cc1]

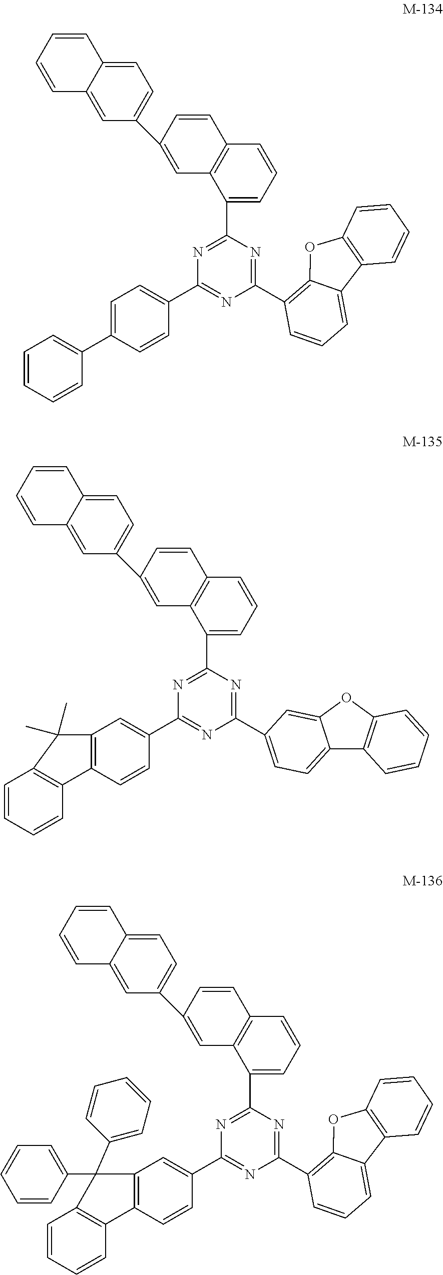 [SMILES: c1ccc(-c2ccc(-c3nc(-c4cccc5ccc(-c6ccc7ccccc7c6)cc45)nc(-c4cccc5c4oc4ccccc45)n3)cc2)cc1 ; CC1(C)c2ccccc2-c2ccc(-c3nc(-c4ccc5c(c4)oc4ccccc45)nc(-c4cccc5ccc(-c6ccc7ccccc7c6)cc45)n3)cc21 ; c1ccc(C2(c3ccccc3)c3ccccc3-c3ccc(-c4nc(-c5cccc6ccc(-c7ccc8ccccc8c7)cc56)nc(-c5cccc6c5oc5ccccc56)n4)cc32)cc1]

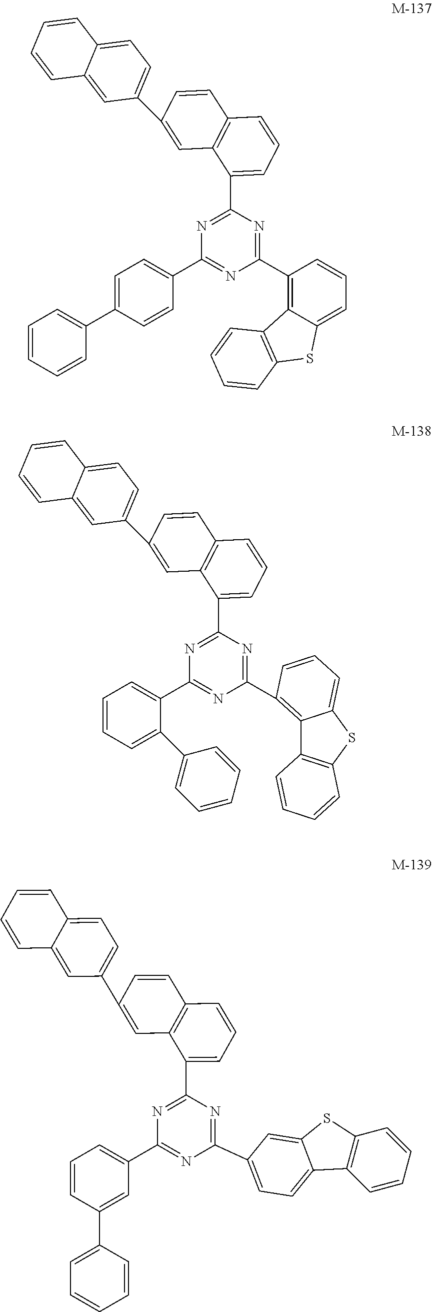 [SMILES: c1ccc(-c2ccc(-c3nc(-c4cccc5ccc(-c6ccc7ccccc7c6)cc45)nc(-c4cccc5sc6ccccc6c45)n3)cc2)cc1 ; c1ccc(-c2ccccc2-c2nc(-c3cccc4ccc(-c5ccc6ccccc6c5)cc34)nc(-c3cccc4sc5ccccc5c34)n2)cc1 ; c1ccc(-c2cccc(-c3nc(-c4ccc5c(c4)sc4ccccc45)nc(-c4cccc5ccc(-c6ccc7ccccc7c6)cc45)n3)c2)cc1]

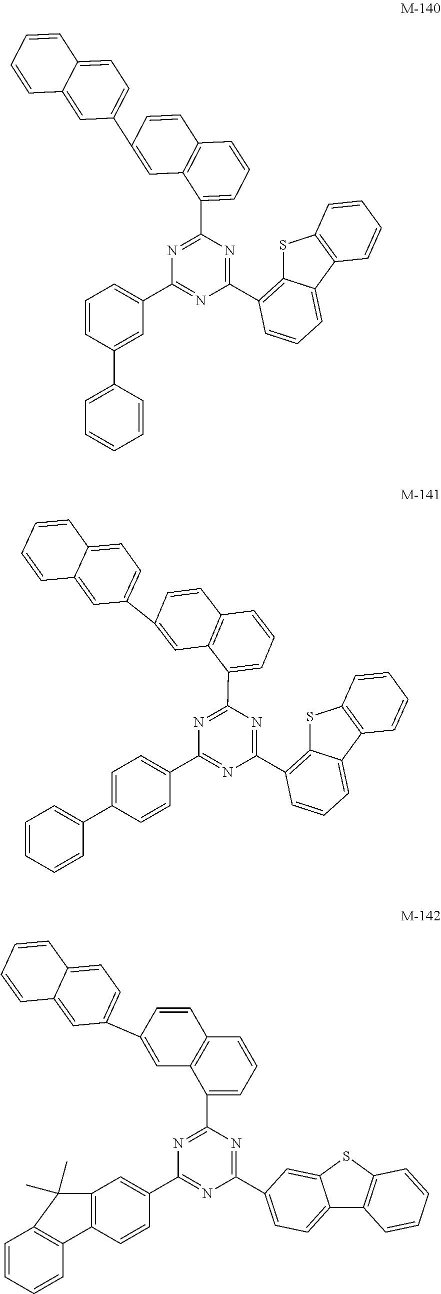 [SMILES: c1ccc(-c2cccc(-c3nc(-c4cccc5ccc(-c6ccc7ccccc7c6)cc45)nc(-c4cccc5c4sc4ccccc45)n3)c2)cc1 ; c1ccc(-c2ccc(-c3nc(-c4cccc5ccc(-c6ccc7ccccc7c6)cc45)nc(-c4cccc5c4sc4ccccc45)n3)cc2)cc1 ; CC1(C)c2ccccc2-c2ccc(-c3nc(-c4ccc5c(c4)sc4ccccc45)nc(-c4cccc5ccc(-c6ccc7ccccc7c6)cc45)n3)cc21]

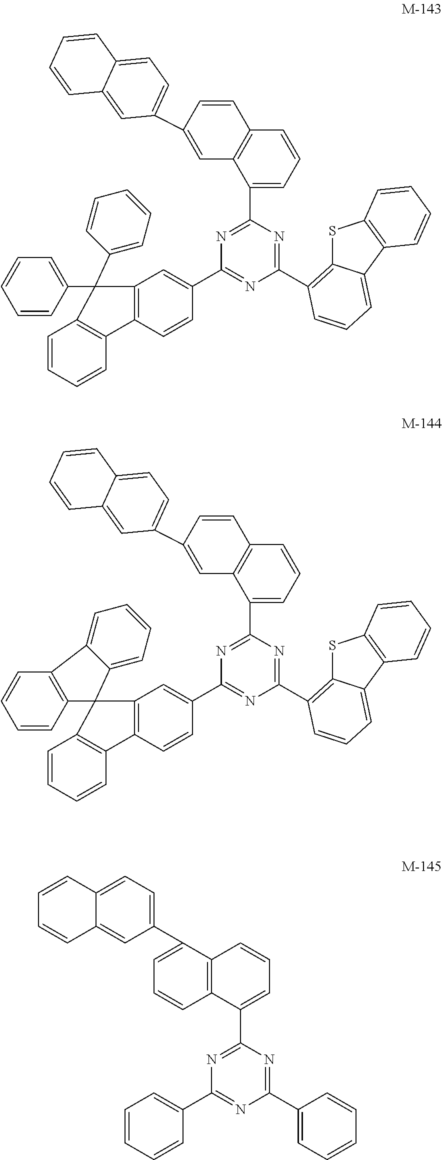 [SMILES: c1ccc(C2(c3ccccc3)c3ccccc3-c3ccc(-c4nc(-c5cccc6ccc(-c7ccc8ccccc8c7)cc56)nc(-c5cccc6c5sc5ccccc56)n4)cc32)cc1 ; c1ccc2c(c1)-c1ccccc1C21c2ccccc2-c2ccc(-c3nc(-c4cccc5ccc(-c6ccc7ccccc7c6)cc45)nc(-c4cccc5c4sc4ccccc45)n3)cc21 ; c1ccc(-c2nc(-c3ccccc3)nc(-c3cccc4c(-c5ccc6ccccc6c5)cccc34)n2)cc1]

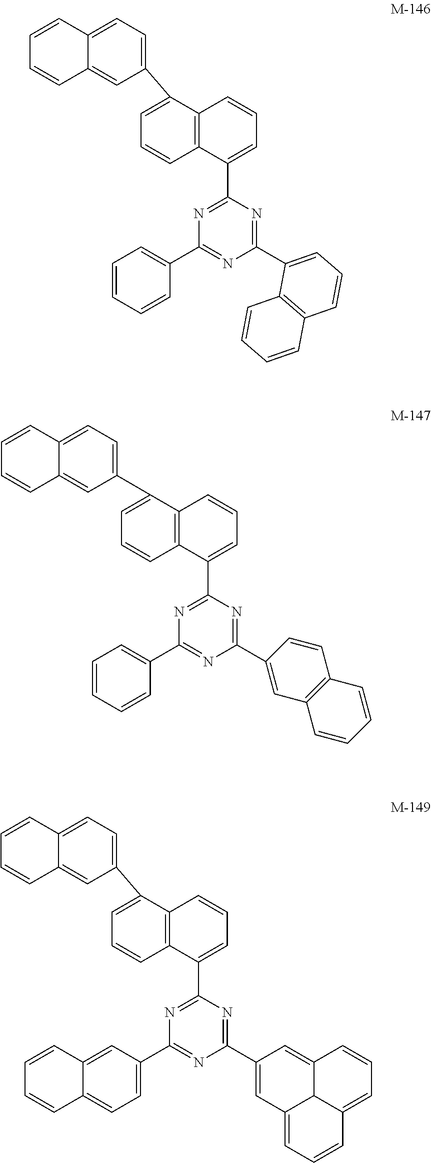 [SMILES: c1ccc(-c2nc(-c3cccc4ccccc34)nc(-c3cccc4c(-c5ccc6ccccc6c5)cccc34)n2)cc1 ; c1ccc(-c2nc(-c3ccc4ccccc4c3)nc(-c3cccc4c(-c5ccc6ccccc6c5)cccc34)n2)cc1 ; C1=CC2=CC=CC3=CC(c4nc(-c5ccc6ccccc6c5)nc(-c5cccc6c(-c7ccc8ccccc8c7)cccc56)n4)=CC(=C1)C23]

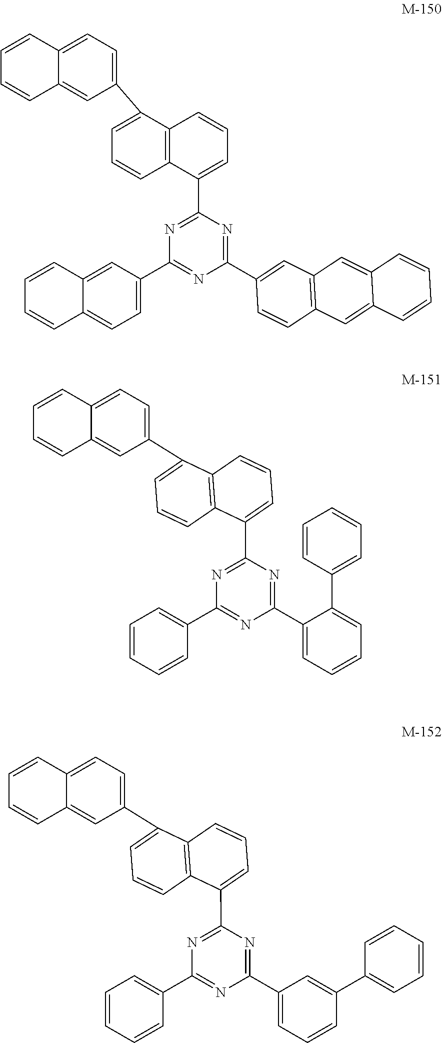 [SMILES: c1ccc2cc(-c3nc(-c4ccc5cc6ccccc6cc5c4)nc(-c4cccc5c(-c6ccc7ccccc7c6)cccc45)n3)ccc2c1 ; c1ccc(-c2nc(-c3ccccc3-c3ccccc3)nc(-c3cccc4c(-c5ccc6ccccc6c5)cccc34)n2)cc1 ; c1ccc(-c2cccc(-c3nc(-c4ccccc4)nc(-c4cccc5c(-c6ccc7ccccc7c6)cccc45)n3)c2)cc1]

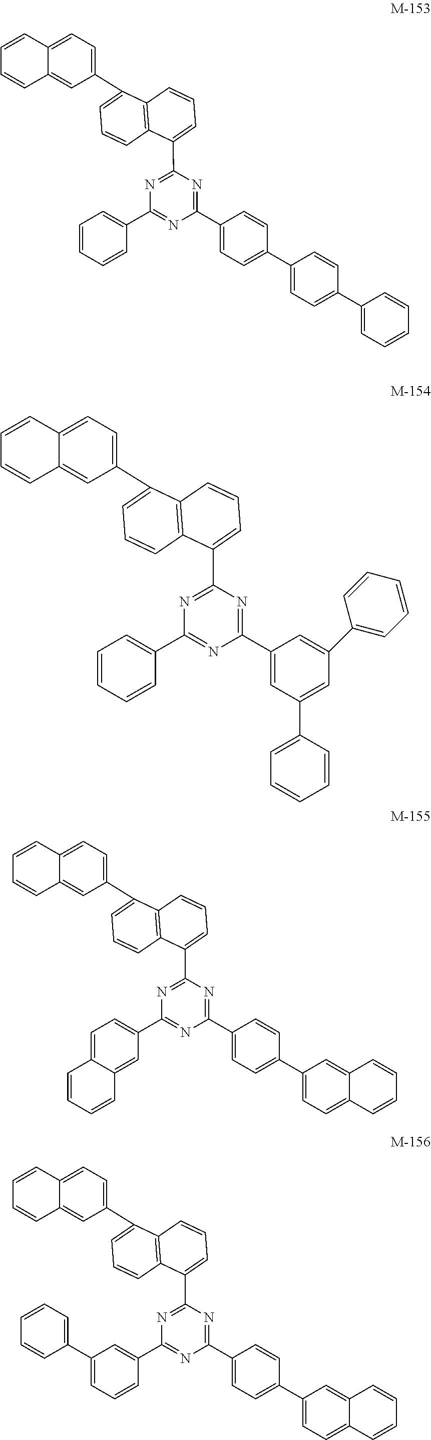 [SMILES: c1ccc(-c2ccc(-c3ccc(-c4nc(-c5ccccc5)nc(-c5cccc6c(-c7ccc8ccccc8c7)cccc56)n4)cc3)cc2)cc1 ; c1ccc(-c2cc(-c3ccccc3)cc(-c3nc(-c4ccccc4)nc(-c4cccc5c(-c6ccc7ccccc7c6)cccc45)n3)c2)cc1 ; c1ccc2cc(-c3ccc(-c4nc(-c5ccc6ccccc6c5)nc(-c5cccc6c(-c7ccc8ccccc8c7)cccc56)n4)cc3)ccc2c1 ; c1ccc(-c2cccc(-c3nc(-c4ccc(-c5ccc6ccccc6c5)cc4)nc(-c4cccc5c(-c6ccc7ccccc7c6)cccc45)n3)c2)cc1]

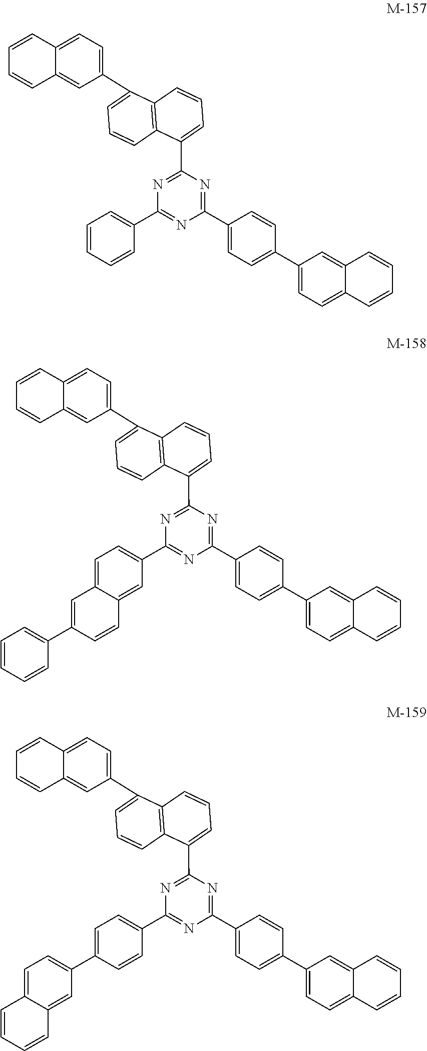 [SMILES: c1ccc(-c2nc(-c3ccc(-c4ccc5ccccc5c4)cc3)nc(-c3cccc4c(-c5ccc6ccccc6c5)cccc34)n2)cc1 ; c1ccc(-c2ccc3cc(-c4nc(-c5ccc(-c6ccc7ccccc7c6)cc5)nc(-c5cccc6c(-c7ccc8ccccc8c7)cccc56)n4)ccc3c2)cc1 ; c1ccc2cc(-c3ccc(-c4nc(-c5ccc(-c6ccc7ccccc7c6)cc5)nc(-c5cccc6c(-c7ccc8ccccc8c7)cccc56)n4)cc3)ccc2c1]

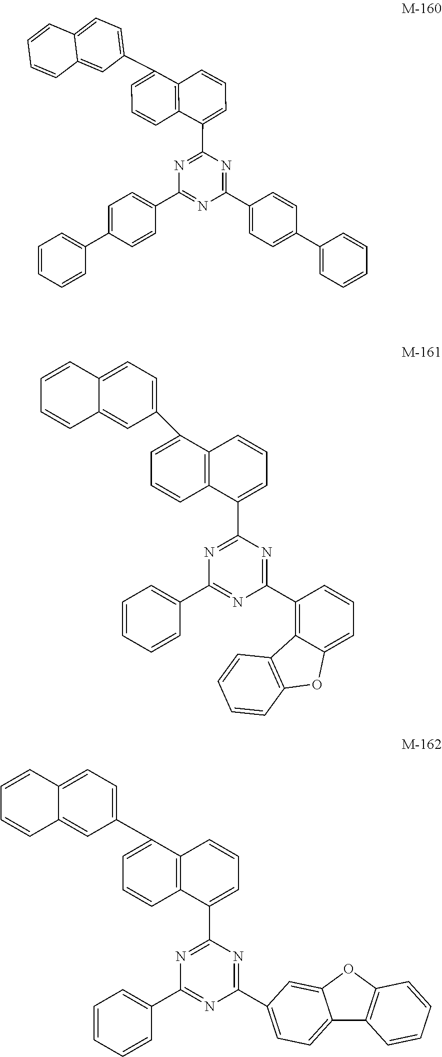 [SMILES: c1ccc(-c2ccc(-c3nc(-c4ccc(-c5ccccc5)cc4)nc(-c4cccc5c(-c6ccc7ccccc7c6)cccc45)n3)cc2)cc1 ; c1ccc(-c2nc(-c3cccc4c(-c5ccc6ccccc6c5)cccc34)nc(-c3cccc4oc5ccccc5c34)n2)cc1 ; c1ccc(-c2nc(-c3ccc4c(c3)oc3ccccc34)nc(-c3cccc4c(-c5ccc6ccccc6c5)cccc34)n2)cc1]

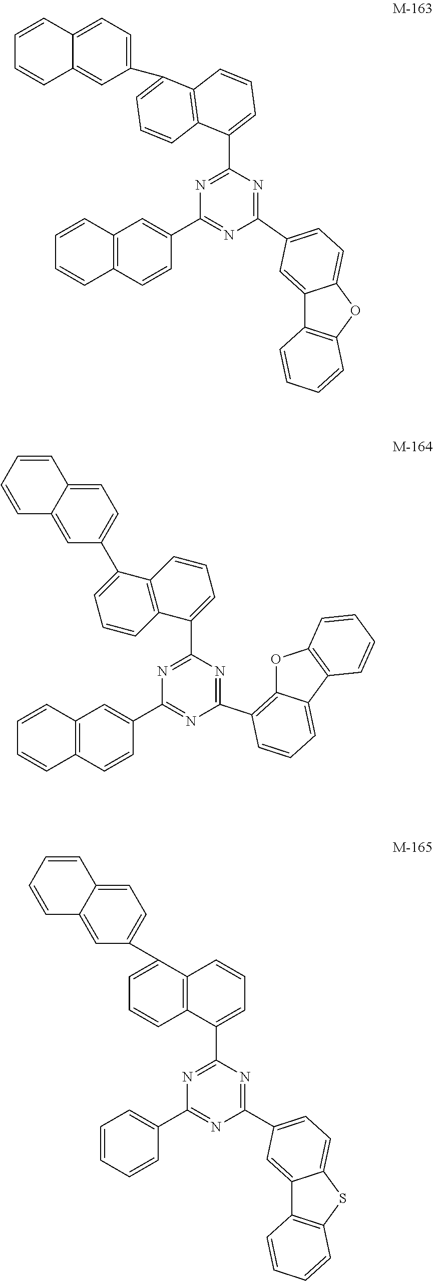 [SMILES: c1ccc2cc(-c3nc(-c4ccc5oc6ccccc6c5c4)nc(-c4cccc5c(-c6ccc7ccccc7c6)cccc45)n3)ccc2c1 ; c1ccc2cc(-c3nc(-c4cccc5c(-c6ccc7ccccc7c6)cccc45)nc(-c4cccc5c4oc4ccccc45)n3)ccc2c1 ; c1ccc(-c2nc(-c3ccc4sc5ccccc5c4c3)nc(-c3cccc4c(-c5ccc6ccccc6c5)cccc34)n2)cc1]

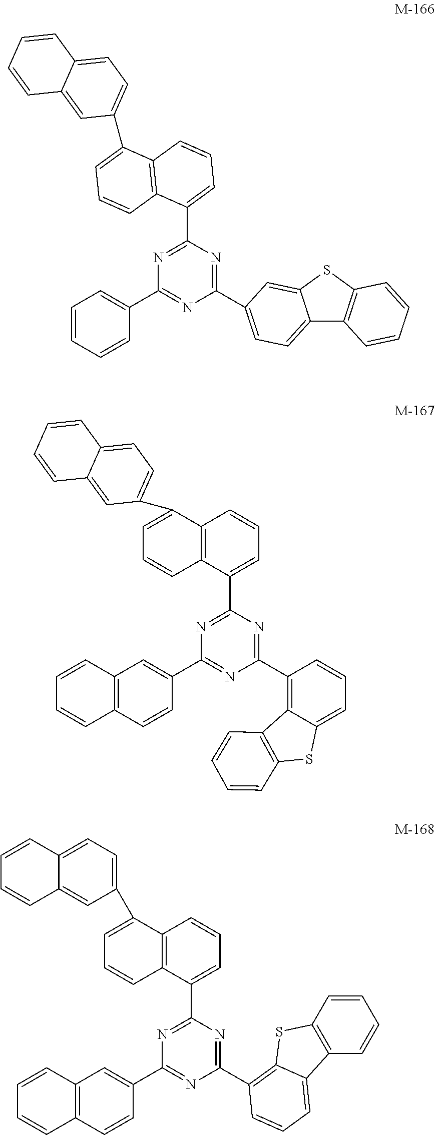 [SMILES: c1ccc(-c2nc(-c3ccc4c(c3)sc3ccccc34)nc(-c3cccc4c(-c5ccc6ccccc6c5)cccc34)n2)cc1 ; c1ccc2cc(-c3nc(-c4cccc5c(-c6ccc7ccccc7c6)cccc45)nc(-c4cccc5sc6ccccc6c45)n3)ccc2c1 ; c1ccc2cc(-c3nc(-c4cccc5c(-c6ccc7ccccc7c6)cccc45)nc(-c4cccc5c4sc4ccccc45)n3)ccc2c1]

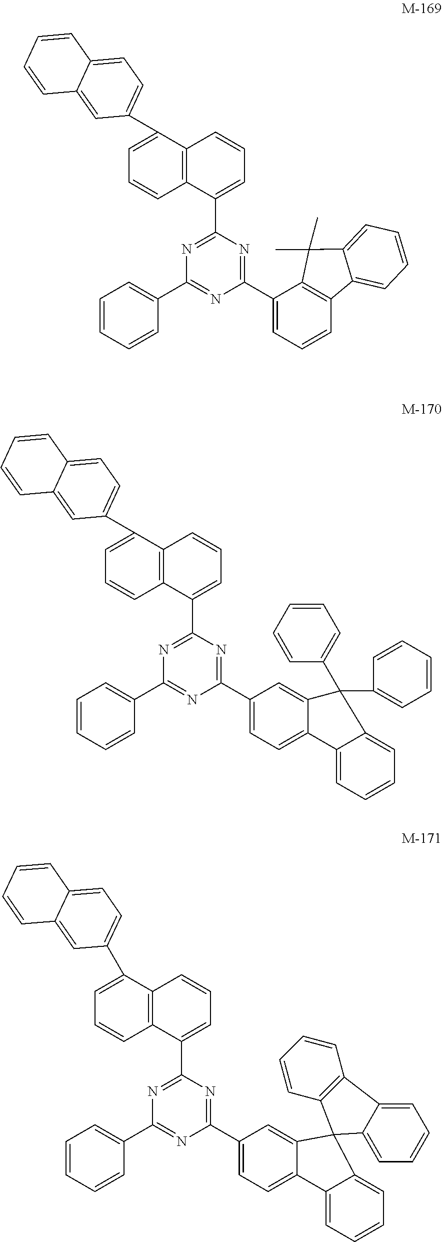 [SMILES: CC1(C)c2ccccc2-c2cccc(-c3nc(-c4ccccc4)nc(-c4cccc5c(-c6ccc7ccccc7c6)cccc45)n3)c21 ; c1ccc(-c2nc(-c3ccc4c(c3)C(c3ccccc3)(c3ccccc3)c3ccccc3-4)nc(-c3cccc4c(-c5ccc6ccccc6c5)cccc34)n2)cc1 ; c1ccc(-c2nc(-c3ccc4c(c3)C3(c5ccccc5-c5ccccc53)c3ccccc3-4)nc(-c3cccc4c(-c5ccc6ccccc6c5)cccc34)n2)cc1]

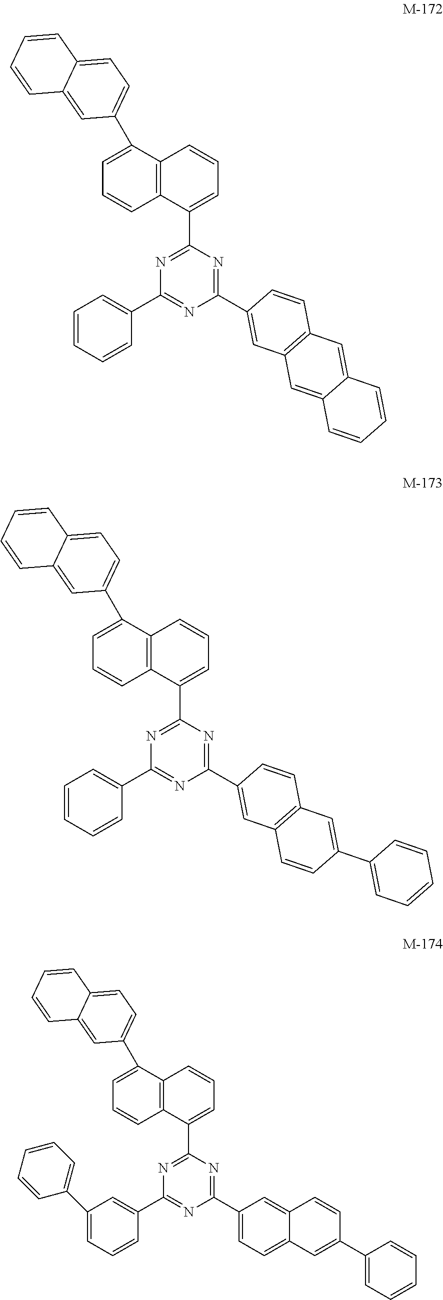 [SMILES: c1ccc(-c2nc(-c3ccc4cc5ccccc5cc4c3)nc(-c3cccc4c(-c5ccc6ccccc6c5)cccc34)n2)cc1 ; c1ccc(-c2ccc3cc(-c4nc(-c5ccccc5)nc(-c5cccc6c(-c7ccc8ccccc8c7)cccc56)n4)ccc3c2)cc1 ; c1ccc(-c2cccc(-c3nc(-c4ccc5cc(-c6ccccc6)ccc5c4)nc(-c4cccc5c(-c6ccc7ccccc7c6)cccc45)n3)c2)cc1]

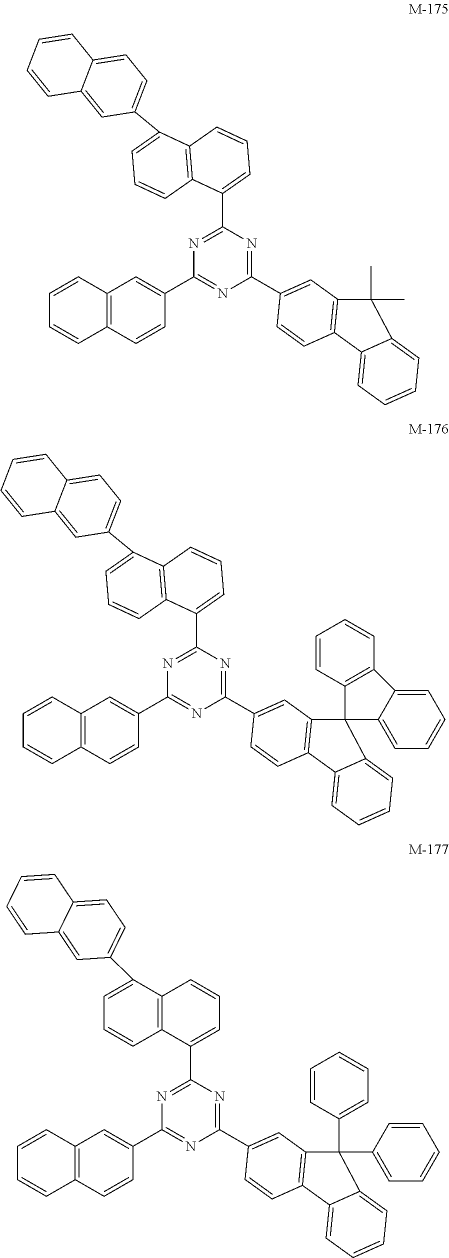 [SMILES: CC1(C)c2ccccc2-c2ccc(-c3nc(-c4ccc5ccccc5c4)nc(-c4cccc5c(-c6ccc7ccccc7c6)cccc45)n3)cc21 ; c1ccc2c(c1)-c1ccccc1C21c2ccccc2-c2ccc(-c3nc(-c4ccc5ccccc5c4)nc(-c4cccc5c(-c6ccc7ccccc7c6)cccc45)n3)cc21 ; c1ccc(C2(c3ccccc3)c3ccccc3-c3ccc(-c4nc(-c5ccc6ccccc6c5)nc(-c5cccc6c(-c7ccc8ccccc8c7)cccc56)n4)cc32)cc1]

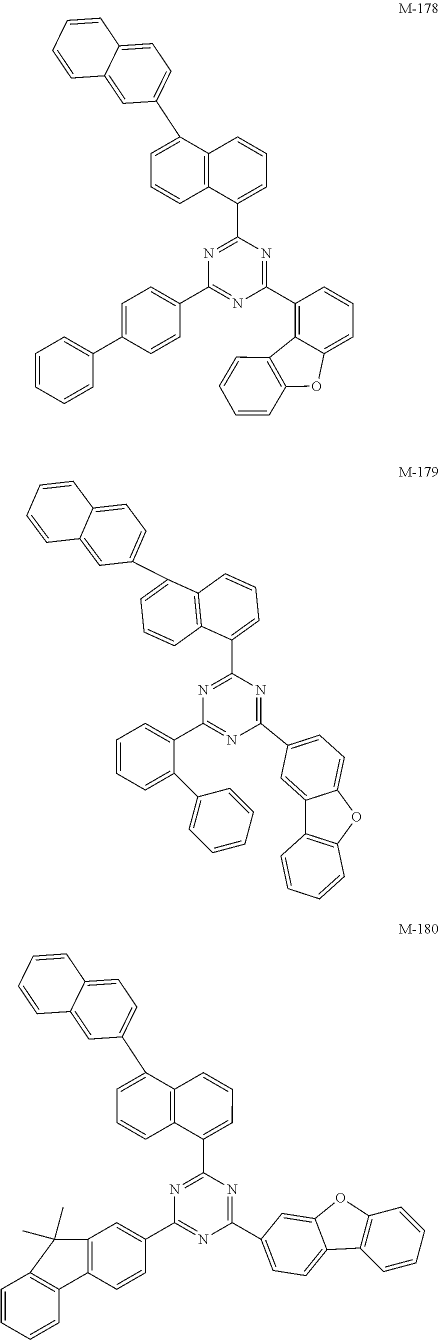 [SMILES: c1ccc(-c2ccc(-c3nc(-c4cccc5c(-c6ccc7ccccc7c6)cccc45)nc(-c4cccc5oc6ccccc6c45)n3)cc2)cc1 ; c1ccc(-c2ccccc2-c2nc(-c3ccc4oc5ccccc5c4c3)nc(-c3cccc4c(-c5ccc6ccccc6c5)cccc34)n2)cc1 ; CC1(C)c2ccccc2-c2ccc(-c3nc(-c4ccc5c(c4)oc4ccccc45)nc(-c4cccc5c(-c6ccc7ccccc7c6)cccc45)n3)cc21]

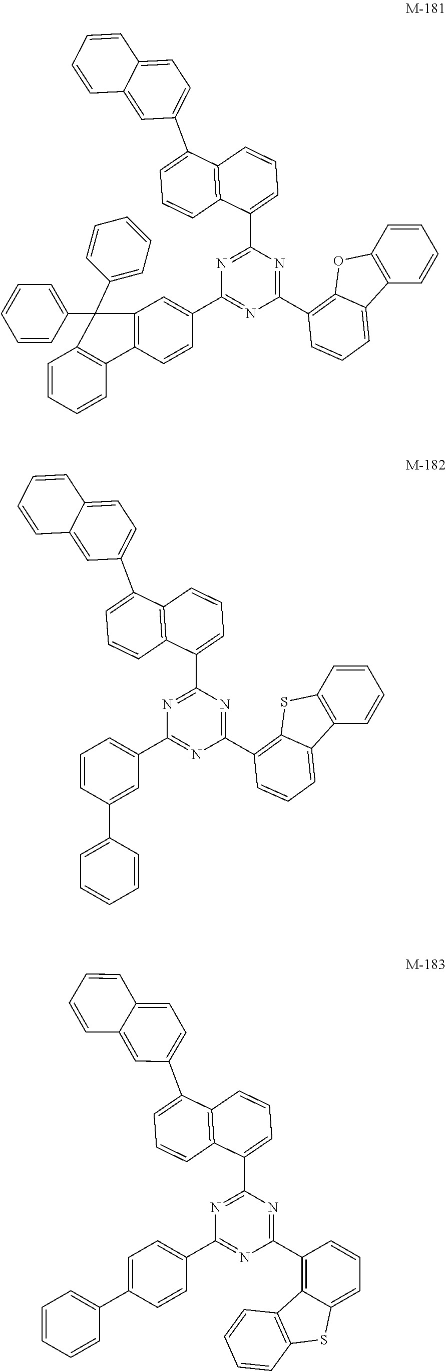 [SMILES: c1ccc(C2(c3ccccc3)c3ccccc3-c3ccc(-c4nc(-c5cccc6c(-c7ccc8ccccc8c7)cccc56)nc(-c5cccc6c5oc5ccccc56)n4)cc32)cc1 ; c1ccc(-c2cccc(-c3nc(-c4cccc5c(-c6ccc7ccccc7c6)cccc45)nc(-c4cccc5c4sc4ccccc45)n3)c2)cc1 ; c1ccc(-c2ccc(-c3nc(-c4cccc5c(-c6ccc7ccccc7c6)cccc45)nc(-c4cccc5sc6ccccc6c45)n3)cc2)cc1]

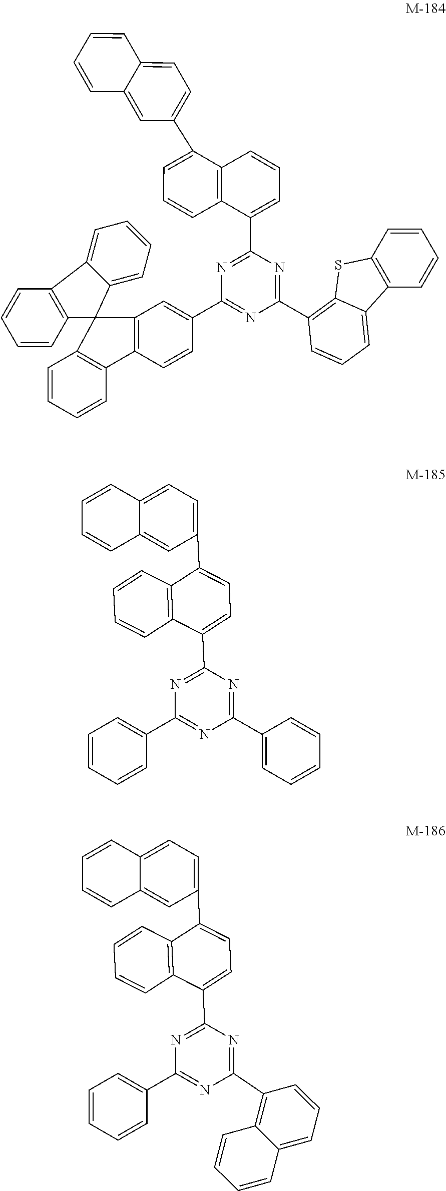 [SMILES: c1ccc2c(c1)-c1ccccc1C21c2ccccc2-c2ccc(-c3nc(-c4cccc5c(-c6ccc7ccccc7c6)cccc45)nc(-c4cccc5c4sc4ccccc45)n3)cc21 ; c1ccc(-c2nc(-c3ccccc3)nc(-c3ccc(-c4ccc5ccccc5c4)c4ccccc34)n2)cc1 ; c1ccc(-c2nc(-c3cccc4ccccc34)nc(-c3ccc(-c4ccc5ccccc5c4)c4ccccc34)n2)cc1]

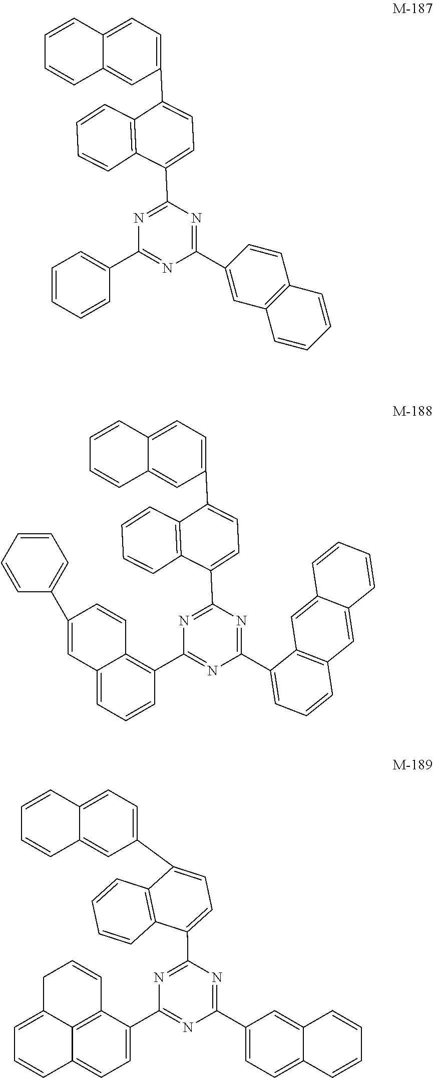 [SMILES: c1ccc(-c2nc(-c3ccc4ccccc4c3)nc(-c3ccc(-c4ccc5ccccc5c4)c4ccccc34)n2)cc1 ; c1ccc(-c2ccc3c(-c4nc(-c5cccc6cc7ccccc7cc56)nc(-c5ccc(-c6ccc7ccccc7c6)c6ccccc56)n4)cccc3c2)cc1 ; C1=Cc2c(-c3nc(-c4ccc5ccccc5c4)nc(-c4ccc(-c5ccc6ccccc6c5)c5ccccc45)n3)ccc3cccc(c23)C1]

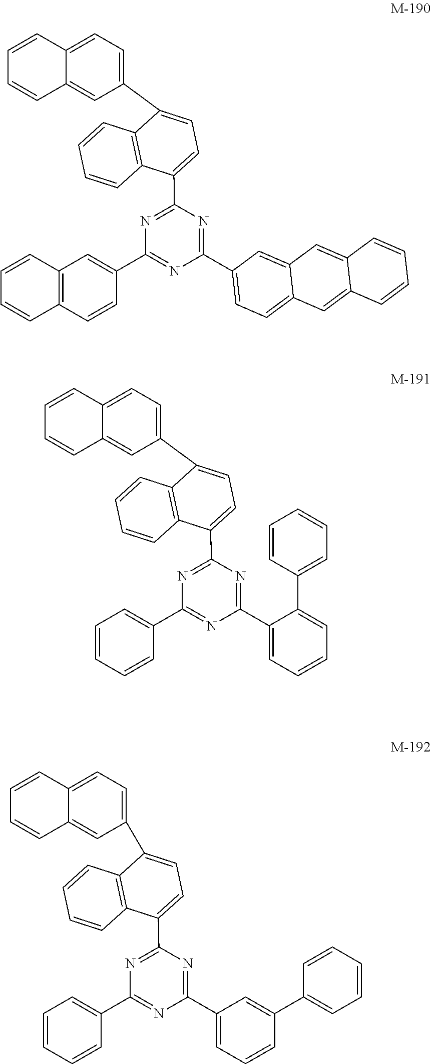 [SMILES: c1ccc2cc(-c3nc(-c4ccc5cc6ccccc6cc5c4)nc(-c4ccc(-c5ccc6ccccc6c5)c5ccccc45)n3)ccc2c1 ; c1ccc(-c2nc(-c3ccccc3-c3ccccc3)nc(-c3ccc(-c4ccc5ccccc5c4)c4ccccc34)n2)cc1 ; c1ccc(-c2cccc(-c3nc(-c4ccccc4)nc(-c4ccc(-c5ccc6ccccc6c5)c5ccccc45)n3)c2)cc1]

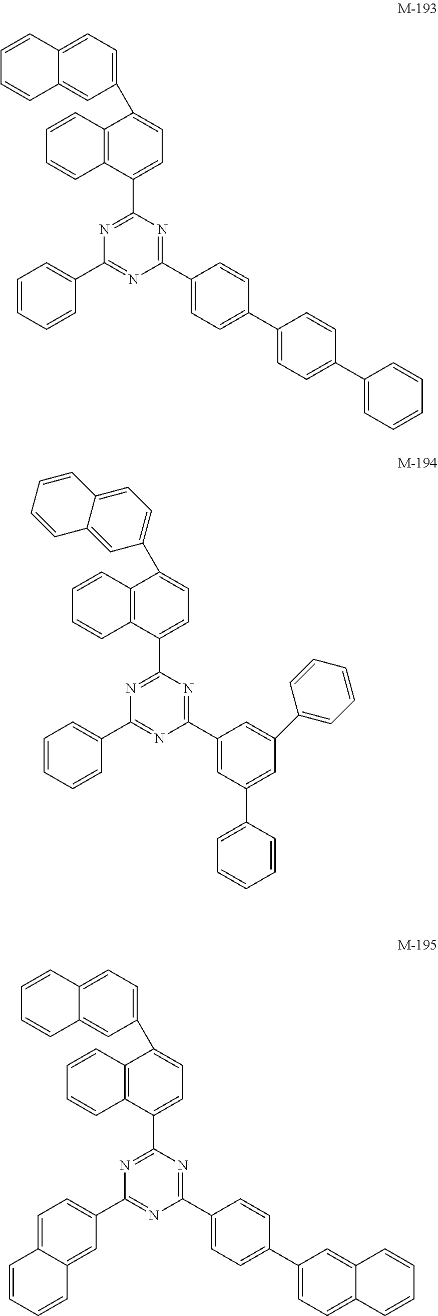 [SMILES: c1ccc(-c2ccc(-c3ccc(-c4nc(-c5ccccc5)nc(-c5ccc(-c6ccc7ccccc7c6)c6ccccc56)n4)cc3)cc2)cc1 ; c1ccc(-c2cc(-c3ccccc3)cc(-c3nc(-c4ccccc4)nc(-c4ccc(-c5ccc6ccccc6c5)c5ccccc45)n3)c2)cc1 ; c1ccc2cc(-c3ccc(-c4nc(-c5ccc6ccccc6c5)nc(-c5ccc(-c6ccc7ccccc7c6)c6ccccc56)n4)cc3)ccc2c1]

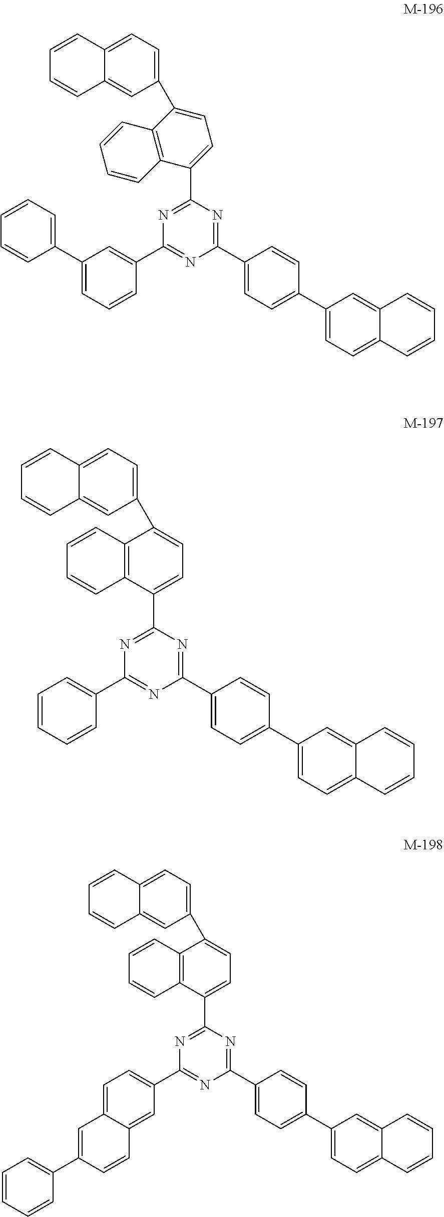 [SMILES: c1ccc(-c2cccc(-c3nc(-c4ccc(-c5ccc6ccccc6c5)cc4)nc(-c4ccc(-c5ccc6ccccc6c5)c5ccccc45)n3)c2)cc1 ; c1ccc(-c2nc(-c3ccc(-c4ccc5ccccc5c4)cc3)nc(-c3ccc(-c4ccc5ccccc5c4)c4ccccc34)n2)cc1 ; c1ccc(-c2ccc3cc(-c4nc(-c5ccc(-c6ccc7ccccc7c6)cc5)nc(-c5ccc(-c6ccc7ccccc7c6)c6ccccc56)n4)ccc3c2)cc1]

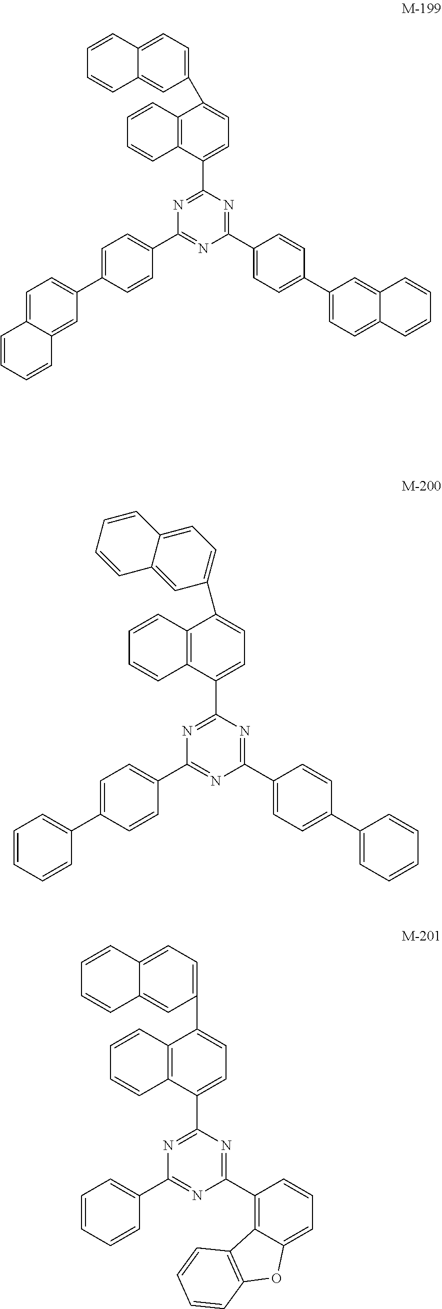

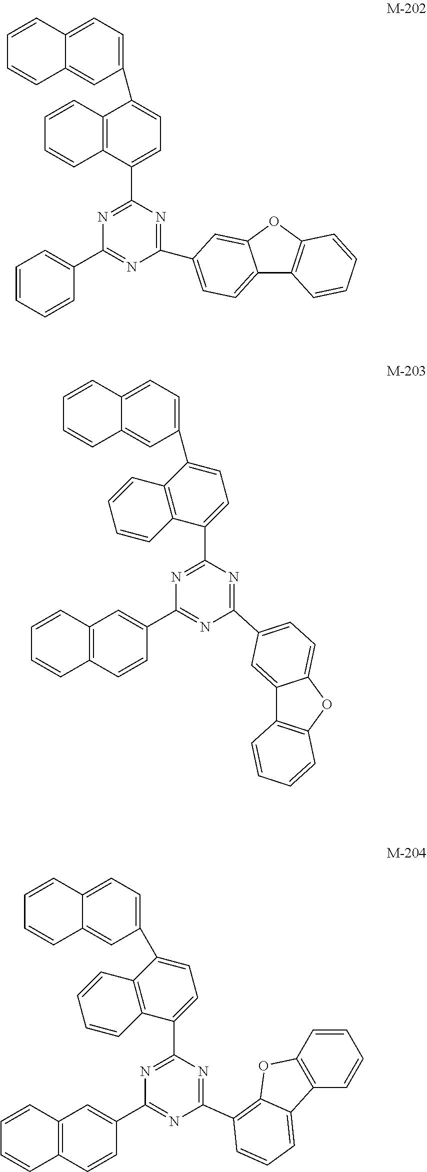 [SMILES: c1ccc(-c2nc(-c3ccc4c(c3)oc3ccccc34)nc(-c3ccc(-c4ccc5ccccc5c4)c4ccccc34)n2)cc1 ; c1ccc2cc(-c3nc(-c4ccc5oc6ccccc6c5c4)nc(-c4ccc(-c5ccc6ccccc6c5)c5ccccc45)n3)ccc2c1 ; c1ccc2cc(-c3nc(-c4ccc(-c5ccc6ccccc6c5)c5ccccc45)nc(-c4cccc5c4oc4ccccc45)n3)ccc2c1]

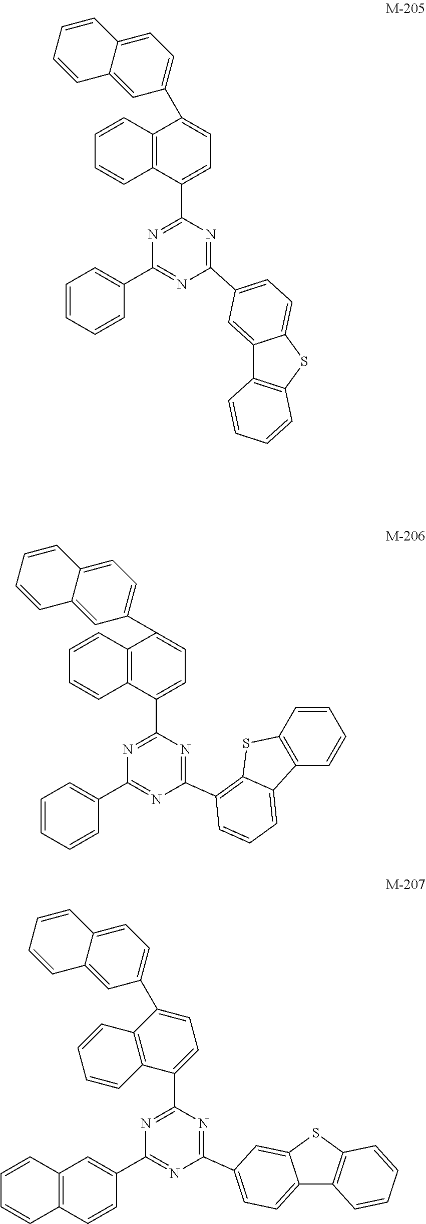 [SMILES: c1ccc(-c2nc(-c3ccc4sc5ccccc5c4c3)nc(-c3ccc(-c4ccc5ccccc5c4)c4ccccc34)n2)cc1 ; c1ccc(-c2nc(-c3ccc(-c4ccc5ccccc5c4)c4ccccc34)nc(-c3cccc4c3sc3ccccc34)n2)cc1 ; c1ccc2cc(-c3nc(-c4ccc5c(c4)sc4ccccc45)nc(-c4ccc(-c5ccc6ccccc6c5)c5ccccc45)n3)ccc2c1]

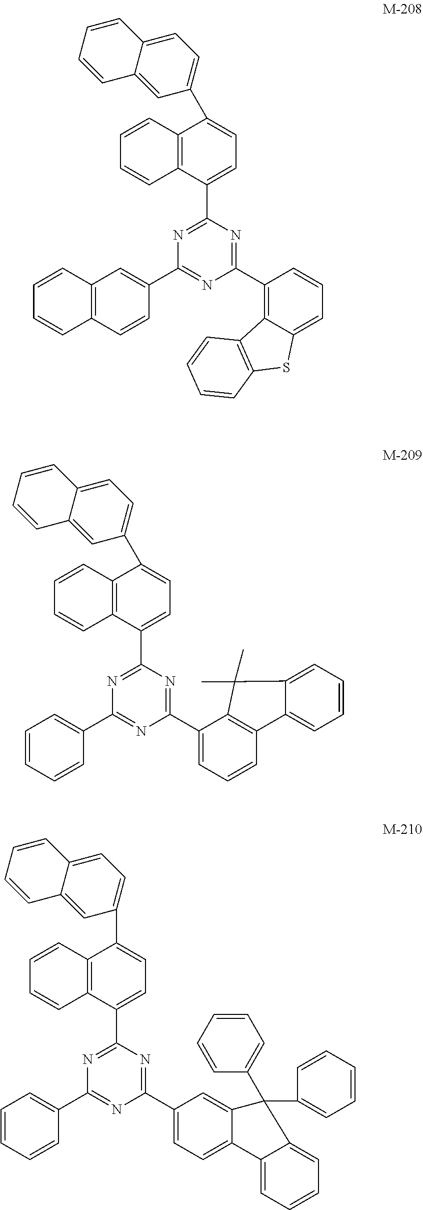 [SMILES: c1ccc2cc(-c3nc(-c4ccc(-c5ccc6ccccc6c5)c5ccccc45)nc(-c4cccc5sc6ccccc6c45)n3)ccc2c1 ; CC1(C)c2ccccc2-c2cccc(-c3nc(-c4ccccc4)nc(-c4ccc(-c5ccc6ccccc6c5)c5ccccc45)n3)c21 ; c1ccc(-c2nc(-c3ccc4c(c3)C(c3ccccc3)(c3ccccc3)c3ccccc3-4)nc(-c3ccc(-c4ccc5ccccc5c4)c4ccccc34)n2)cc1]

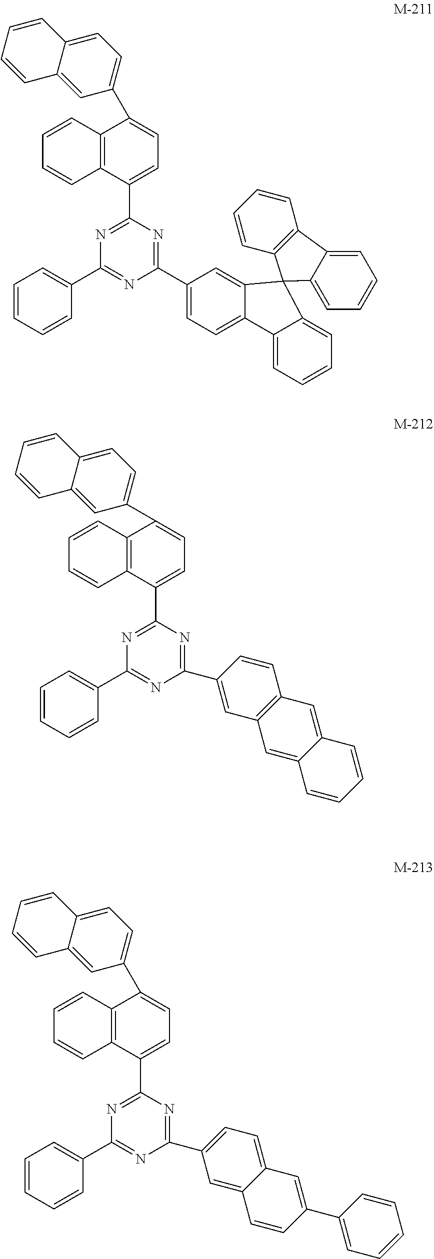 [SMILES: c1ccc(-c2nc(-c3ccc4c(c3)C3(c5ccccc5-c5ccccc53)c3ccccc3-4)nc(-c3ccc(-c4ccc5ccccc5c4)c4ccccc34)n2)cc1 ; c1ccc(-c2nc(-c3ccc4cc5ccccc5cc4c3)nc(-c3ccc(-c4ccc5ccccc5c4)c4ccccc34)n2)cc1 ; c1ccc(-c2ccc3cc(-c4nc(-c5ccccc5)nc(-c5ccc(-c6ccc7ccccc7c6)c6ccccc56)n4)ccc3c2)cc1]

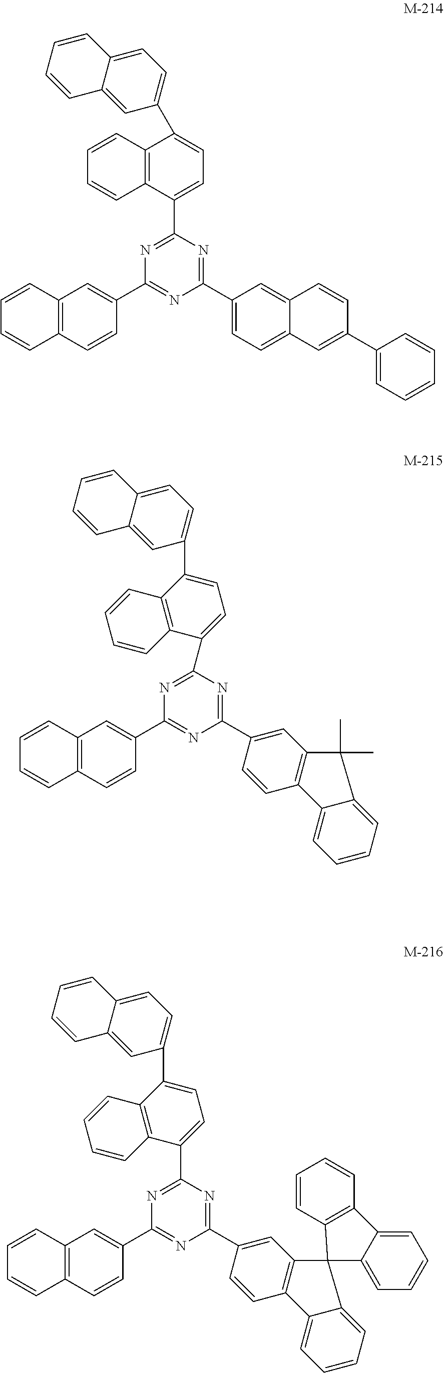 [SMILES: c1ccc(-c2ccc3cc(-c4nc(-c5ccc6ccccc6c5)nc(-c5ccc(-c6ccc7ccccc7c6)c6ccccc56)n4)ccc3c2)cc1 ; CC1(C)c2ccccc2-c2ccc(-c3nc(-c4ccc5ccccc5c4)nc(-c4ccc(-c5ccc6ccccc6c5)c5ccccc45)n3)cc21 ; c1ccc2c(c1)-c1ccccc1C21c2ccccc2-c2ccc(-c3nc(-c4ccc5ccccc5c4)nc(-c4ccc(-c5ccc6ccccc6c5)c5ccccc45)n3)cc21]

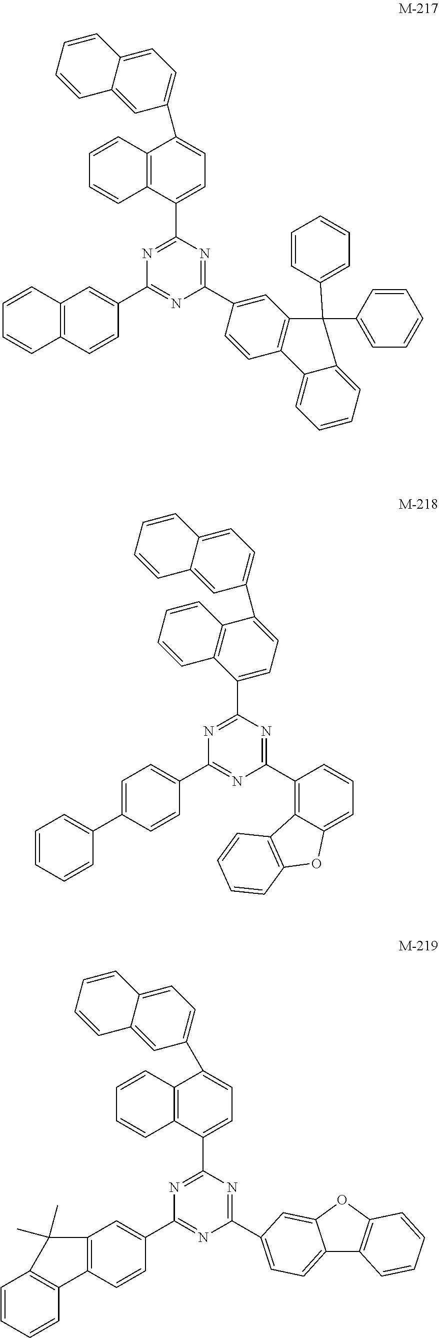 [SMILES: c1ccc(C2(c3ccccc3)c3ccccc3-c3ccc(-c4nc(-c5ccc6ccccc6c5)nc(-c5ccc(-c6ccc7ccccc7c6)c6ccccc56)n4)cc32)cc1 ; c1ccc(-c2ccc(-c3nc(-c4ccc(-c5ccc6ccccc6c5)c5ccccc45)nc(-c4cccc5oc6ccccc6c45)n3)cc2)cc1 ; CC1(C)c2ccccc2-c2ccc(-c3nc(-c4ccc5c(c4)oc4ccccc45)nc(-c4ccc(-c5ccc6ccccc6c5)c5ccccc45)n3)cc21]

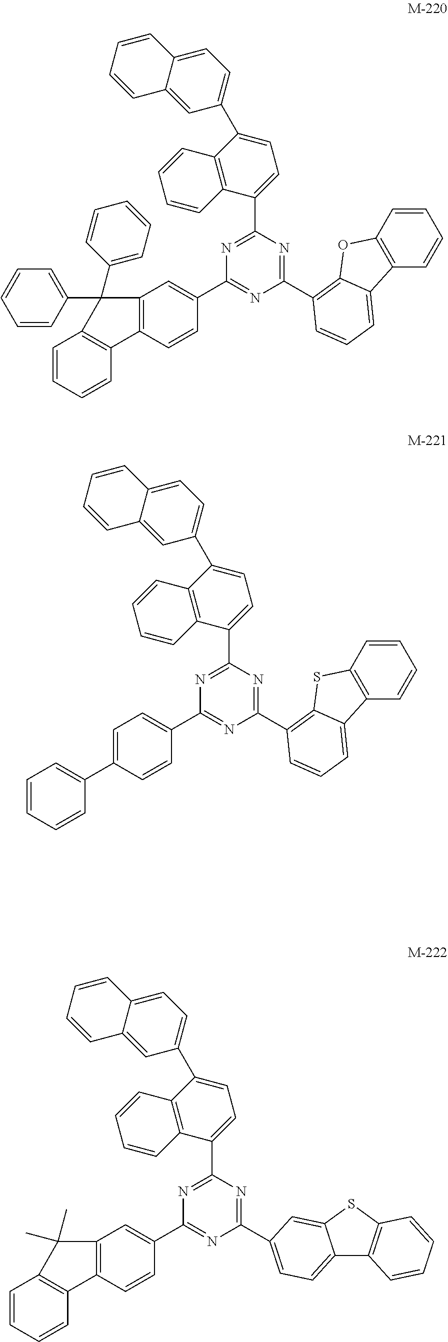 [SMILES: c1ccc(C2(c3ccccc3)c3ccccc3-c3ccc(-c4nc(-c5ccc(-c6ccc7ccccc7c6)c6ccccc56)nc(-c5cccc6c5oc5ccccc56)n4)cc32)cc1 ; c1ccc(-c2ccc(-c3nc(-c4ccc(-c5ccc6ccccc6c5)c5ccccc45)nc(-c4cccc5c4sc4ccccc45)n3)cc2)cc1 ; CC1(C)c2ccccc2-c2ccc(-c3nc(-c4ccc5c(c4)sc4ccccc45)nc(-c4ccc(-c5ccc6ccccc6c5)c5ccccc45)n3)cc21]

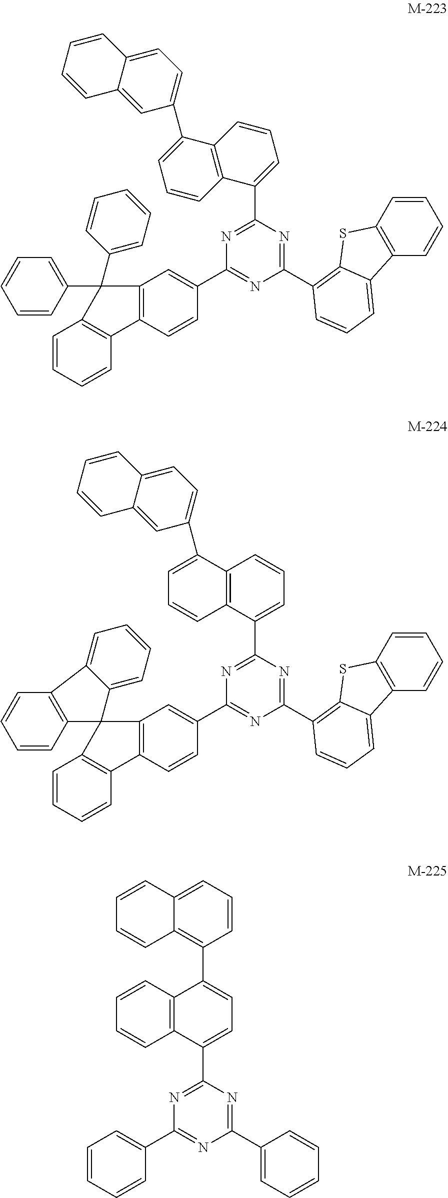 [SMILES: c1ccc(C2(c3ccccc3)c3ccccc3-c3ccc(-c4nc(-c5cccc6c(-c7ccc8ccccc8c7)cccc56)nc(-c5cccc6c5sc5ccccc56)n4)cc32)cc1 ; c1ccc2c(c1)-c1ccccc1C21c2ccccc2-c2ccc(-c3nc(-c4cccc5c(-c6ccc7ccccc7c6)cccc45)nc(-c4cccc5c4sc4ccccc45)n3)cc21 ; c1ccc(-c2nc(-c3ccccc3)nc(-c3ccc(-c4cccc5ccccc45)c4ccccc34)n2)cc1]

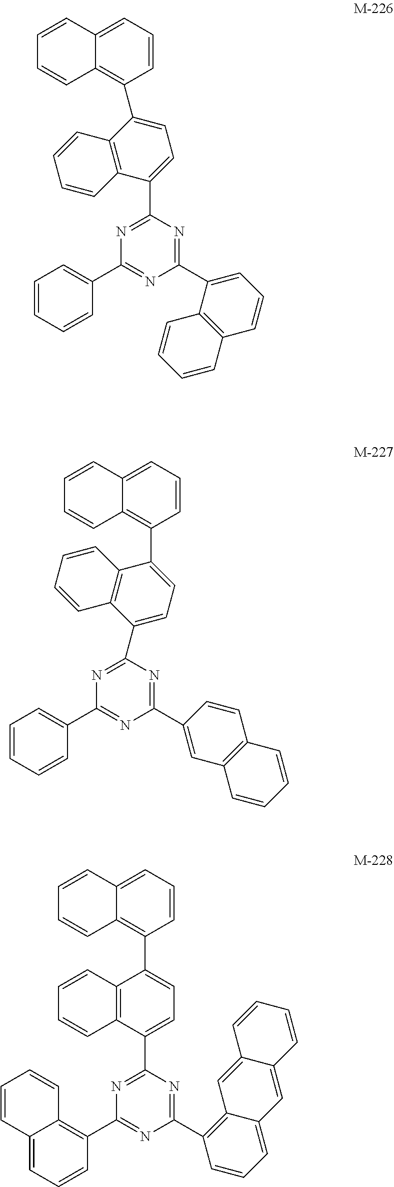 [SMILES: c1ccc(-c2nc(-c3cccc4ccccc34)nc(-c3ccc(-c4cccc5ccccc45)c4ccccc34)n2)cc1 ; c1ccc(-c2nc(-c3ccc4ccccc4c3)nc(-c3ccc(-c4cccc5ccccc45)c4ccccc34)n2)cc1 ; c1ccc2cc3c(-c4nc(-c5cccc6ccccc56)nc(-c5ccc(-c6cccc7ccccc67)c6ccccc56)n4)cccc3cc2c1]

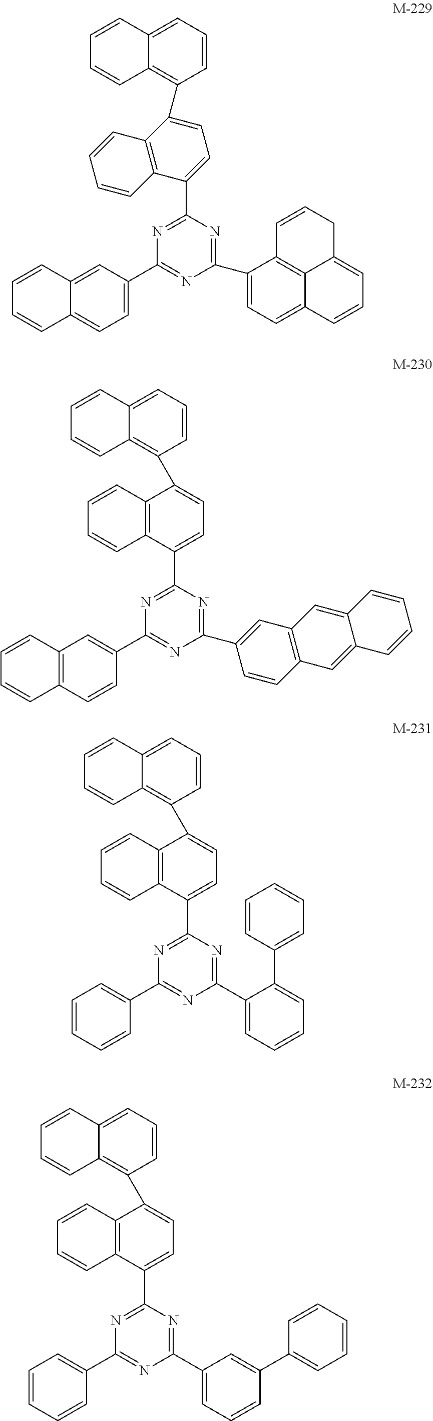

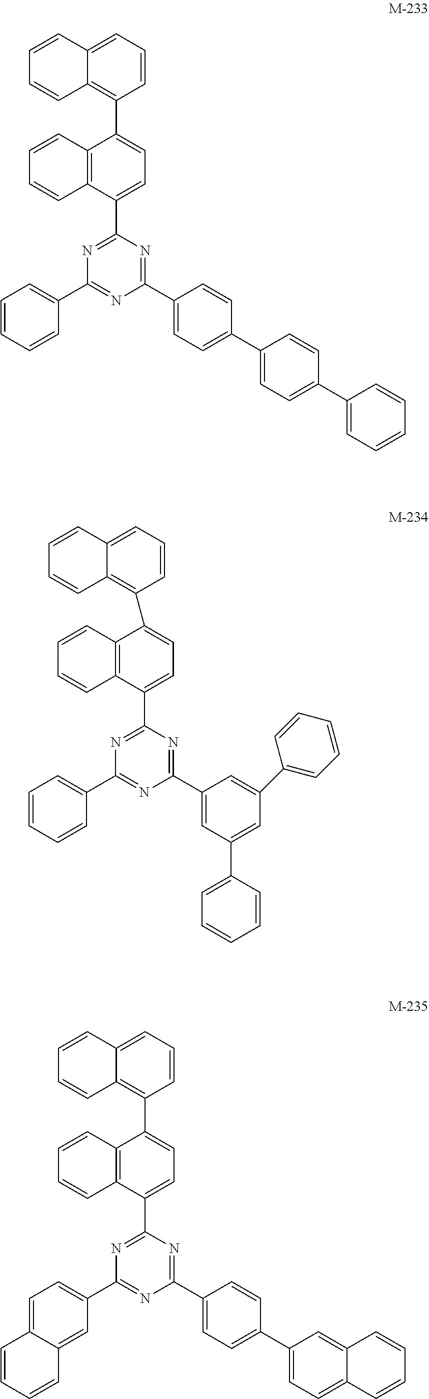 [SMILES: c1ccc(-c2ccc(-c3ccc(-c4nc(-c5ccccc5)nc(-c5ccc(-c6cccc7ccccc67)c6ccccc56)n4)cc3)cc2)cc1 ; c1ccc(-c2cc(-c3ccccc3)cc(-c3nc(-c4ccccc4)nc(-c4ccc(-c5cccc6ccccc56)c5ccccc45)n3)c2)cc1 ; c1ccc2cc(-c3ccc(-c4nc(-c5ccc6ccccc6c5)nc(-c5ccc(-c6cccc7ccccc67)c6ccccc56)n4)cc3)ccc2c1]

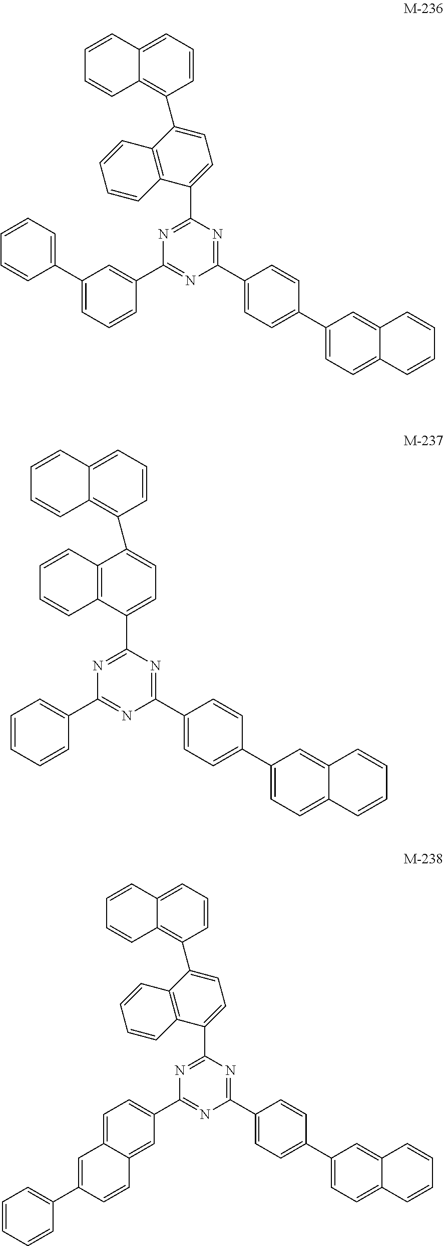 [SMILES: c1ccc(-c2cccc(-c3nc(-c4ccc(-c5ccc6ccccc6c5)cc4)nc(-c4ccc(-c5cccc6ccccc56)c5ccccc45)n3)c2)cc1 ; c1ccc(-c2nc(-c3ccc(-c4ccc5ccccc5c4)cc3)nc(-c3ccc(-c4cccc5ccccc45)c4ccccc34)n2)cc1 ; c1ccc(-c2ccc3cc(-c4nc(-c5ccc(-c6ccc7ccccc7c6)cc5)nc(-c5ccc(-c6cccc7ccccc67)c6ccccc56)n4)ccc3c2)cc1]

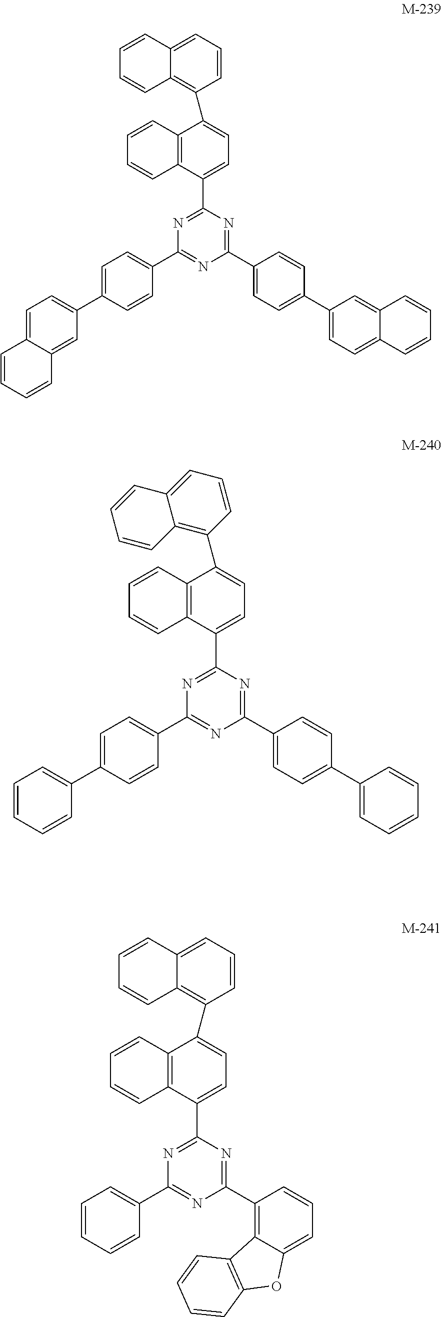 [SMILES: c1ccc2cc(-c3ccc(-c4nc(-c5ccc(-c6ccc7ccccc7c6)cc5)nc(-c5ccc(-c6cccc7ccccc67)c6ccccc56)n4)cc3)ccc2c1 ; c1ccc(-c2ccc(-c3nc(-c4ccc(-c5ccccc5)cc4)nc(-c4ccc(-c5cccc6ccccc56)c5ccccc45)n3)cc2)cc1 ; c1ccc(-c2nc(-c3ccc(-c4cccc5ccccc45)c4ccccc34)nc(-c3cccc4oc5ccccc5c34)n2)cc1]

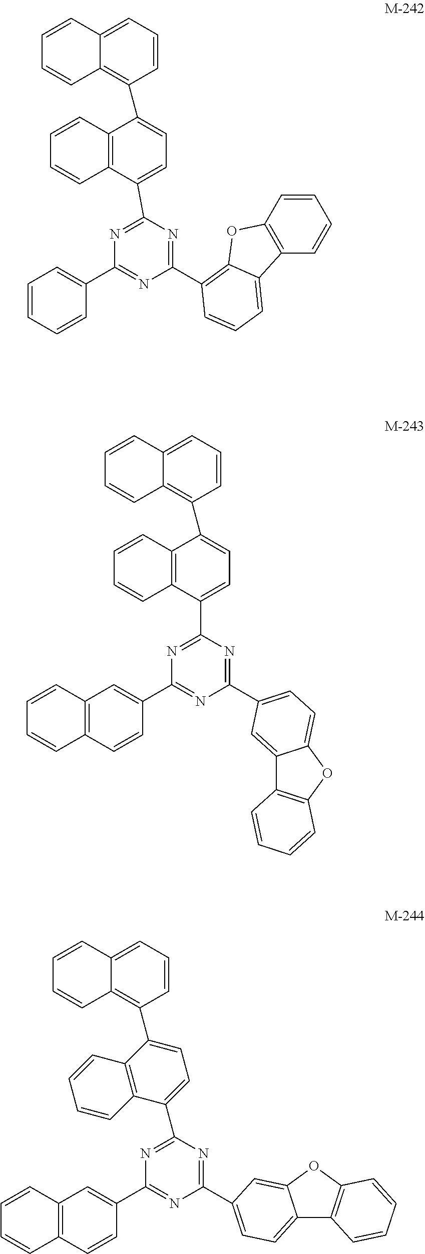 [SMILES: c1ccc(-c2nc(-c3ccc(-c4cccc5ccccc45)c4ccccc34)nc(-c3cccc4c3oc3ccccc34)n2)cc1 ; c1ccc2cc(-c3nc(-c4ccc5oc6ccccc6c5c4)nc(-c4ccc(-c5cccc6ccccc56)c5ccccc45)n3)ccc2c1 ; c1ccc2cc(-c3nc(-c4ccc5c(c4)oc4ccccc45)nc(-c4ccc(-c5cccc6ccccc56)c5ccccc45)n3)ccc2c1]

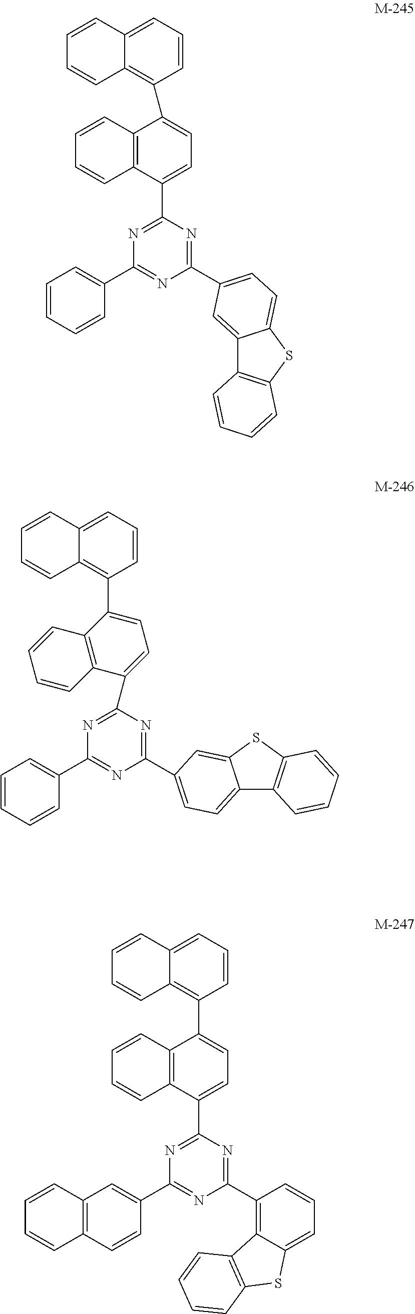 [SMILES: c1ccc(-c2nc(-c3ccc4sc5ccccc5c4c3)nc(-c3ccc(-c4cccc5ccccc45)c4ccccc34)n2)cc1 ; c1ccc(-c2nc(-c3ccc4c(c3)sc3ccccc34)nc(-c3ccc(-c4cccc5ccccc45)c4ccccc34)n2)cc1 ; c1ccc2cc(-c3nc(-c4ccc(-c5cccc6ccccc56)c5ccccc45)nc(-c4cccc5sc6ccccc6c45)n3)ccc2c1]

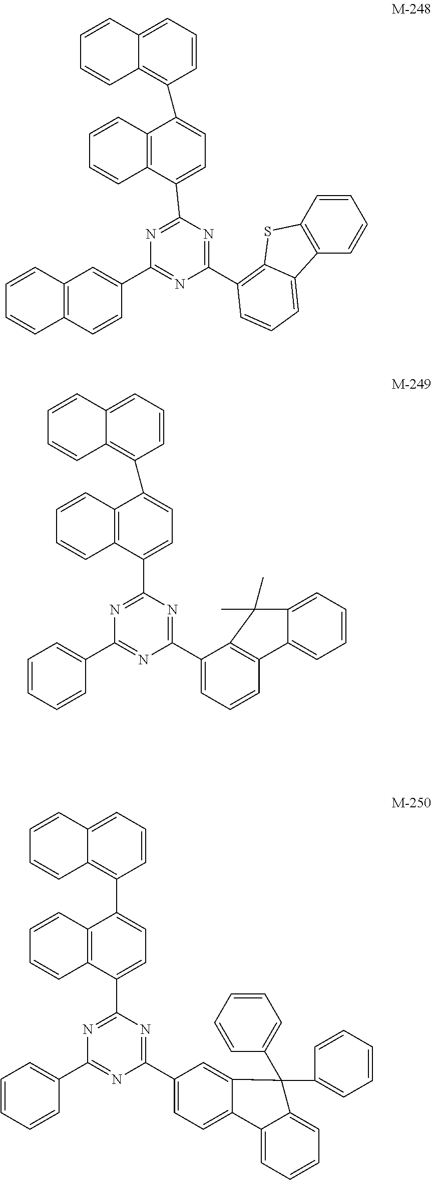 [SMILES: c1ccc2cc(-c3nc(-c4ccc(-c5cccc6ccccc56)c5ccccc45)nc(-c4cccc5c4sc4ccccc45)n3)ccc2c1 ; CC1(C)c2ccccc2-c2cccc(-c3nc(-c4ccccc4)nc(-c4ccc(-c5cccc6ccccc56)c5ccccc45)n3)c21 ; c1ccc(-c2nc(-c3ccc4c(c3)C(c3ccccc3)(c3ccccc3)c3ccccc3-4)nc(-c3ccc(-c4cccc5ccccc45)c4ccccc34)n2)cc1]

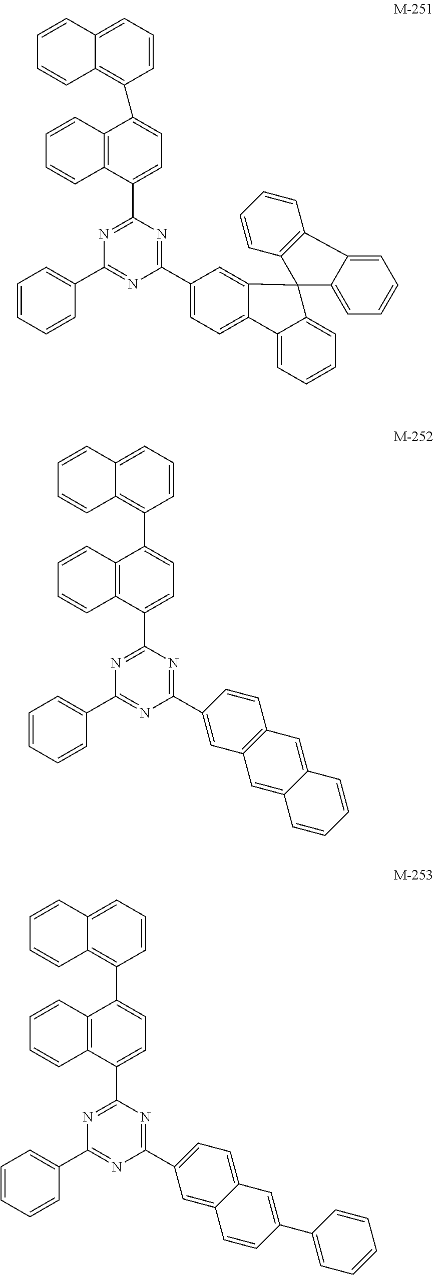 [SMILES: c1ccc(-c2nc(-c3ccc4c(c3)C3(c5ccccc5-c5ccccc53)c3ccccc3-4)nc(-c3ccc(-c4cccc5ccccc45)c4ccccc34)n2)cc1 ; c1ccc(-c2nc(-c3ccc4cc5ccccc5cc4c3)nc(-c3ccc(-c4cccc5ccccc45)c4ccccc34)n2)cc1 ; c1ccc(-c2ccc3cc(-c4nc(-c5ccccc5)nc(-c5ccc(-c6cccc7ccccc67)c6ccccc56)n4)ccc3c2)cc1]

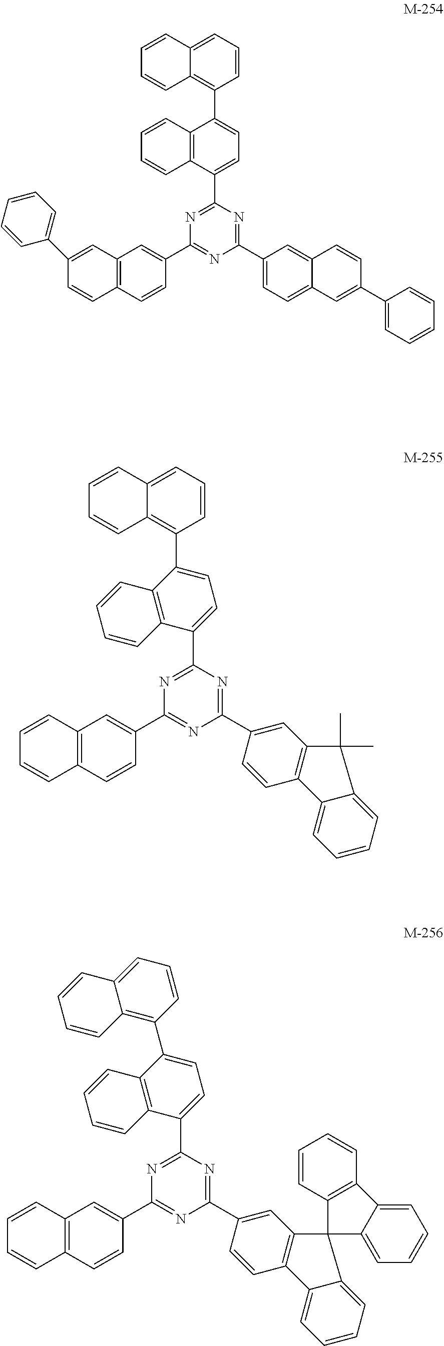 [SMILES: c1ccc(-c2ccc3cc(-c4nc(-c5ccc6ccc(-c7ccccc7)cc6c5)nc(-c5ccc(-c6cccc7ccccc67)c6ccccc56)n4)ccc3c2)cc1 ; CC1(C)c2ccccc2-c2ccc(-c3nc(-c4ccc5ccccc5c4)nc(-c4ccc(-c5cccc6ccccc56)c5ccccc45)n3)cc21 ; c1ccc2c(c1)-c1ccccc1C21c2ccccc2-c2ccc(-c3nc(-c4ccc5ccccc5c4)nc(-c4ccc(-c5cccc6ccccc56)c5ccccc45)n3)cc21]

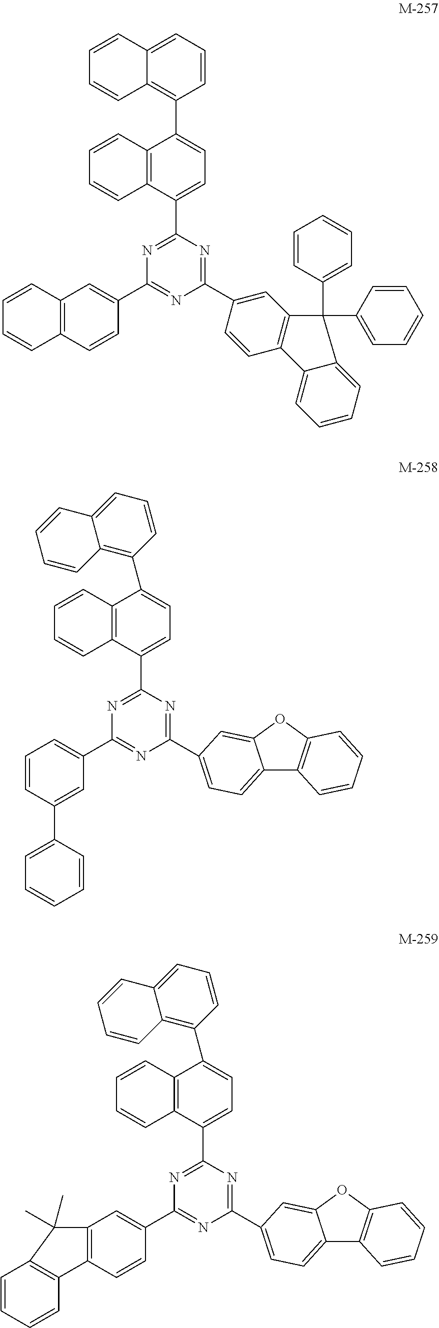 [SMILES: c1ccc(C2(c3ccccc3)c3ccccc3-c3ccc(-c4nc(-c5ccc6ccccc6c5)nc(-c5ccc(-c6cccc7ccccc67)c6ccccc56)n4)cc32)cc1 ; c1ccc(-c2cccc(-c3nc(-c4ccc5c(c4)oc4ccccc45)nc(-c4ccc(-c5cccc6ccccc56)c5ccccc45)n3)c2)cc1 ; CC1(C)c2ccccc2-c2ccc(-c3nc(-c4ccc5c(c4)oc4ccccc45)nc(-c4ccc(-c5cccc6ccccc56)c5ccccc45)n3)cc21]

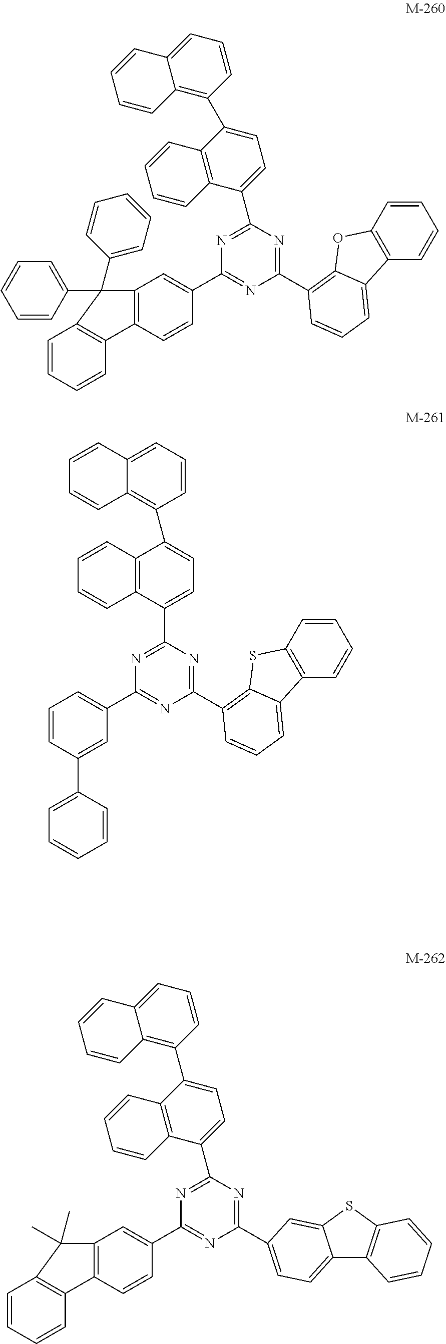 [SMILES: c1ccc(C2(c3ccccc3)c3ccccc3-c3ccc(-c4nc(-c5ccc(-c6cccc7ccccc67)c6ccccc56)nc(-c5cccc6c5oc5ccccc56)n4)cc32)cc1 ; c1ccc(-c2cccc(-c3nc(-c4ccc(-c5cccc6ccccc56)c5ccccc45)nc(-c4cccc5c4sc4ccccc45)n3)c2)cc1 ; CC1(C)c2ccccc2-c2ccc(-c3nc(-c4ccc5c(c4)sc4ccccc45)nc(-c4ccc(-c5cccc6ccccc56)c5ccccc45)n3)cc21]

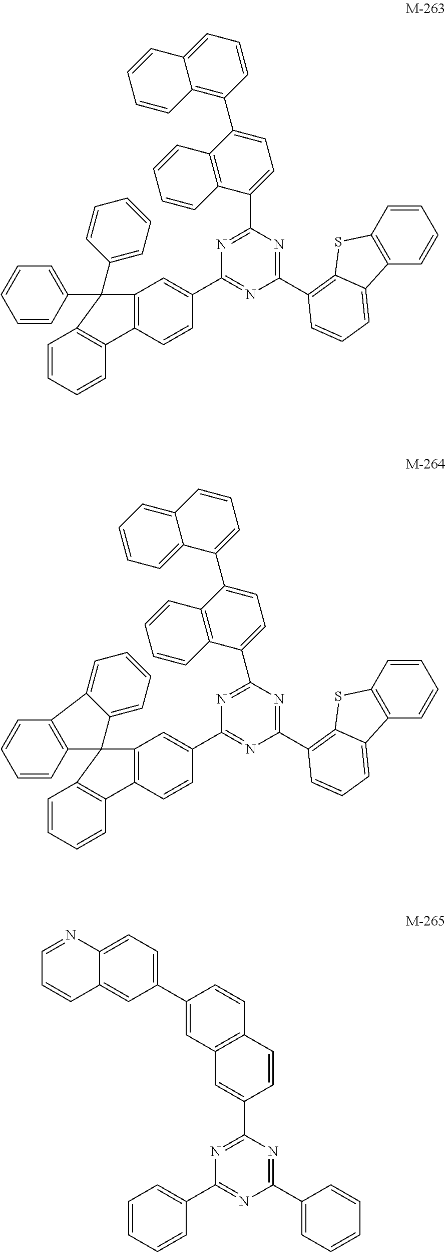 [SMILES: c1ccc(C2(c3ccccc3)c3ccccc3-c3ccc(-c4nc(-c5ccc(-c6cccc7ccccc67)c6ccccc56)nc(-c5cccc6c5sc5ccccc56)n4)cc32)cc1 ; c1ccc2c(c1)-c1ccccc1C21c2ccccc2-c2ccc(-c3nc(-c4ccc(-c5cccc6ccccc56)c5ccccc45)nc(-c4cccc5c4sc4ccccc45)n3)cc21 ; c1ccc(-c2nc(-c3ccccc3)nc(-c3ccc4ccc(-c5ccc6ncccc6c5)cc4c3)n2)cc1]

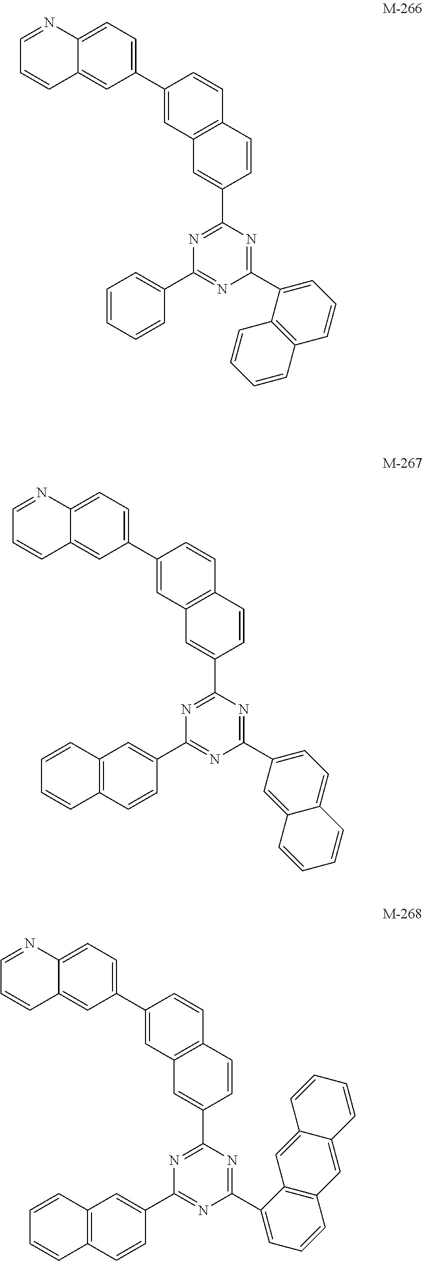 [SMILES: c1ccc(-c2nc(-c3ccc4ccc(-c5ccc6ncccc6c5)cc4c3)nc(-c3cccc4ccccc34)n2)cc1 ; c1ccc2cc(-c3nc(-c4ccc5ccccc5c4)nc(-c4ccc5ccc(-c6ccc7ncccc7c6)cc5c4)n3)ccc2c1 ; c1ccc2cc(-c3nc(-c4ccc5ccc(-c6ccc7ncccc7c6)cc5c4)nc(-c4cccc5cc6ccccc6cc45)n3)ccc2c1]

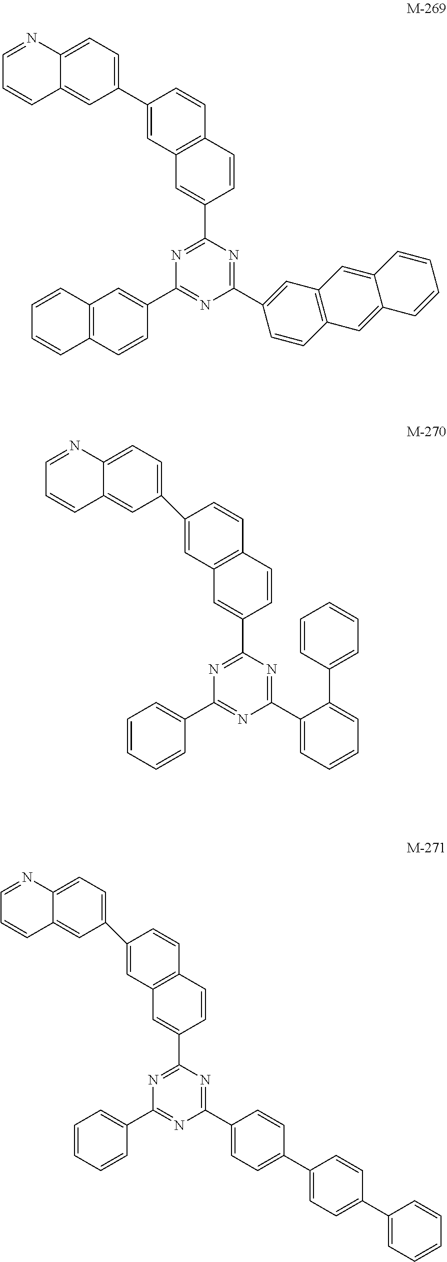 [SMILES: c1ccc2cc(-c3nc(-c4ccc5ccc(-c6ccc7ncccc7c6)cc5c4)nc(-c4ccc5cc6ccccc6cc5c4)n3)ccc2c1 ; c1ccc(-c2nc(-c3ccc4ccc(-c5ccc6ncccc6c5)cc4c3)nc(-c3ccccc3-c3ccccc3)n2)cc1 ; c1ccc(-c2ccc(-c3ccc(-c4nc(-c5ccccc5)nc(-c5ccc6ccc(-c7ccc8ncccc8c7)cc6c5)n4)cc3)cc2)cc1]

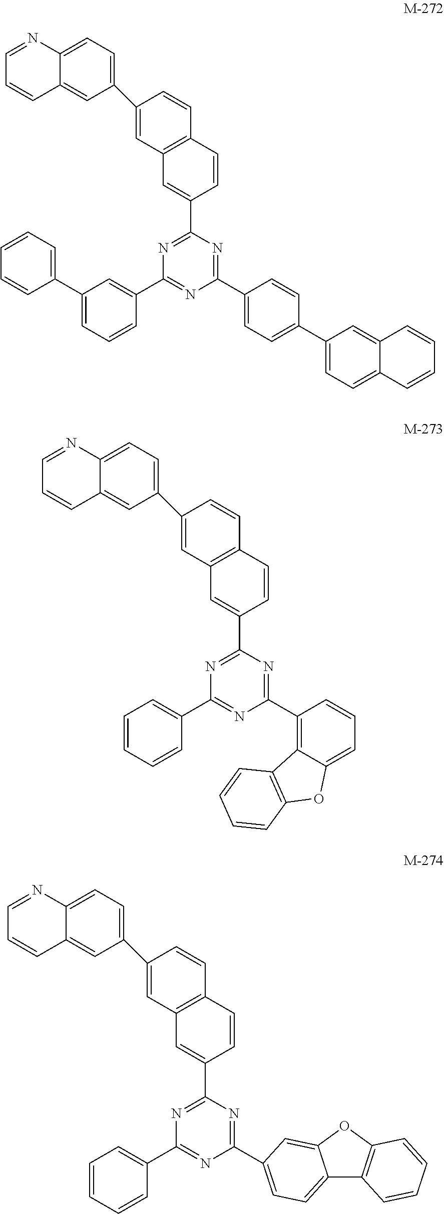 [SMILES: c1ccc(-c2cccc(-c3nc(-c4ccc(-c5ccc6ccccc6c5)cc4)nc(-c4ccc5ccc(-c6ccc7ncccc7c6)cc5c4)n3)c2)cc1 ; c1ccc(-c2nc(-c3ccc4ccc(-c5ccc6ncccc6c5)cc4c3)nc(-c3cccc4oc5ccccc5c34)n2)cc1 ; c1ccc(-c2nc(-c3ccc4ccc(-c5ccc6ncccc6c5)cc4c3)nc(-c3ccc4c(c3)oc3ccccc34)n2)cc1]

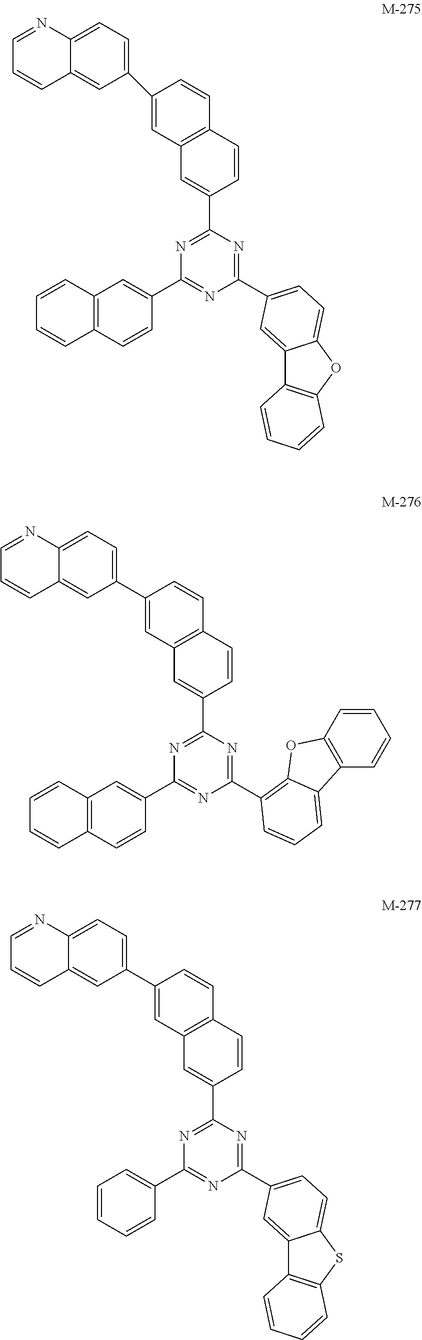 [SMILES: c1ccc2cc(-c3nc(-c4ccc5ccc(-c6ccc7ncccc7c6)cc5c4)nc(-c4ccc5oc6ccccc6c5c4)n3)ccc2c1 ; c1ccc2cc(-c3nc(-c4ccc5ccc(-c6ccc7ncccc7c6)cc5c4)nc(-c4cccc5c4oc4ccccc45)n3)ccc2c1 ; c1ccc(-c2nc(-c3ccc4ccc(-c5ccc6ncccc6c5)cc4c3)nc(-c3ccc4sc5ccccc5c4c3)n2)cc1]

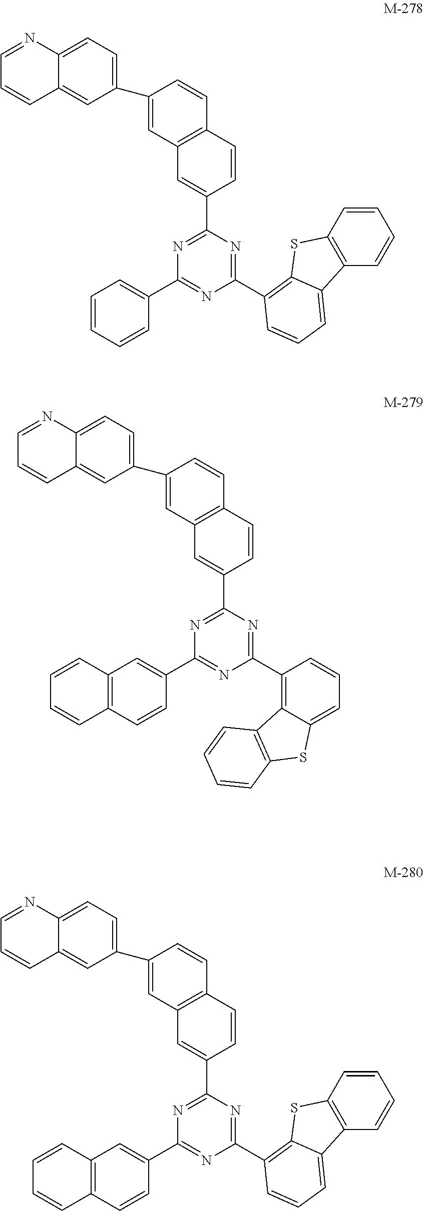 [SMILES: c1ccc(-c2nc(-c3ccc4ccc(-c5ccc6ncccc6c5)cc4c3)nc(-c3cccc4c3sc3ccccc34)n2)cc1 ; c1ccc2cc(-c3nc(-c4ccc5ccc(-c6ccc7ncccc7c6)cc5c4)nc(-c4cccc5sc6ccccc6c45)n3)ccc2c1 ; c1ccc2cc(-c3nc(-c4ccc5ccc(-c6ccc7ncccc7c6)cc5c4)nc(-c4cccc5c4sc4ccccc45)n3)ccc2c1]

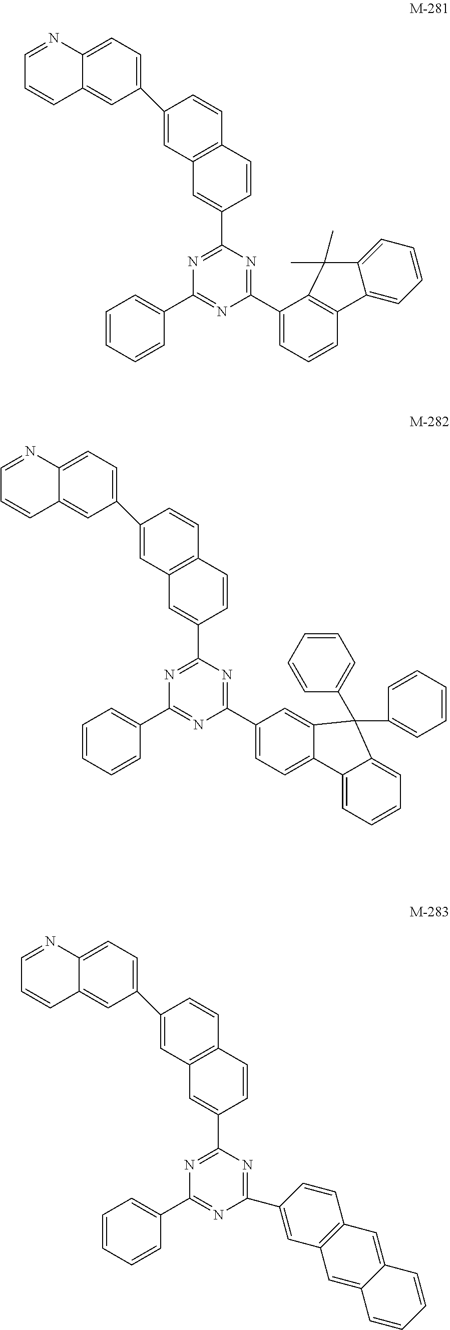 [SMILES: CC1(C)c2ccccc2-c2cccc(-c3nc(-c4ccccc4)nc(-c4ccc5ccc(-c6ccc7ncccc7c6)cc5c4)n3)c21 ; c1ccc(-c2nc(-c3ccc4c(c3)C(c3ccccc3)(c3ccccc3)c3ccccc3-4)nc(-c3ccc4ccc(-c5ccc6ncccc6c5)cc4c3)n2)cc1 ; c1ccc(-c2nc(-c3ccc4ccc(-c5ccc6ncccc6c5)cc4c3)nc(-c3ccc4cc5ccccc5cc4c3)n2)cc1]

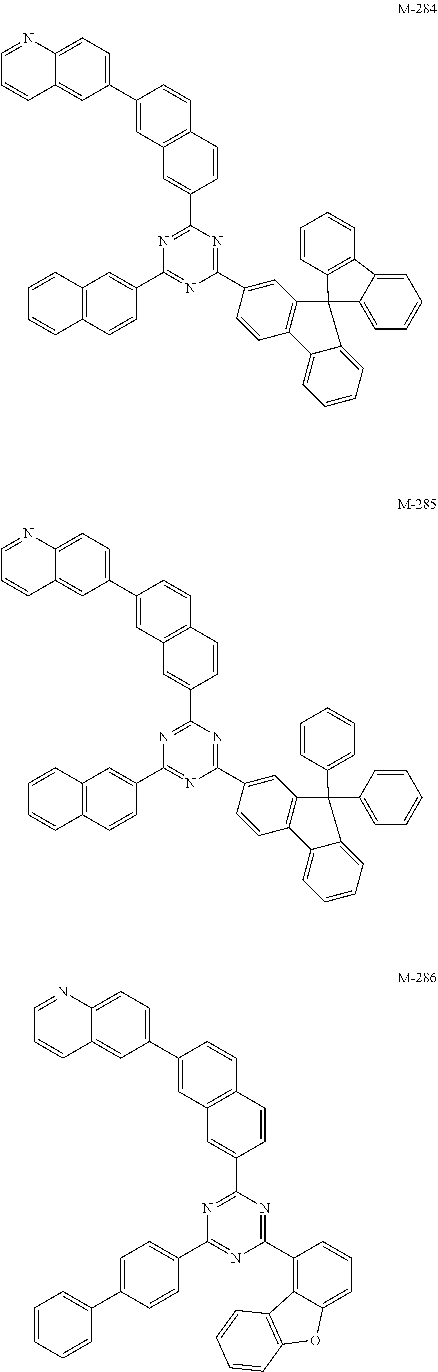 [SMILES: c1ccc2c(c1)-c1ccccc1C21c2ccccc2-c2ccc(-c3nc(-c4ccc5ccccc5c4)nc(-c4ccc5ccc(-c6ccc7ncccc7c6)cc5c4)n3)cc21 ; c1ccc(C2(c3ccccc3)c3ccccc3-c3ccc(-c4nc(-c5ccc6ccccc6c5)nc(-c5ccc6ccc(-c7ccc8ncccc8c7)cc6c5)n4)cc32)cc1 ; c1ccc(-c2ccc(-c3nc(-c4ccc5ccc(-c6ccc7ncccc7c6)cc5c4)nc(-c4cccc5oc6ccccc6c45)n3)cc2)cc1]

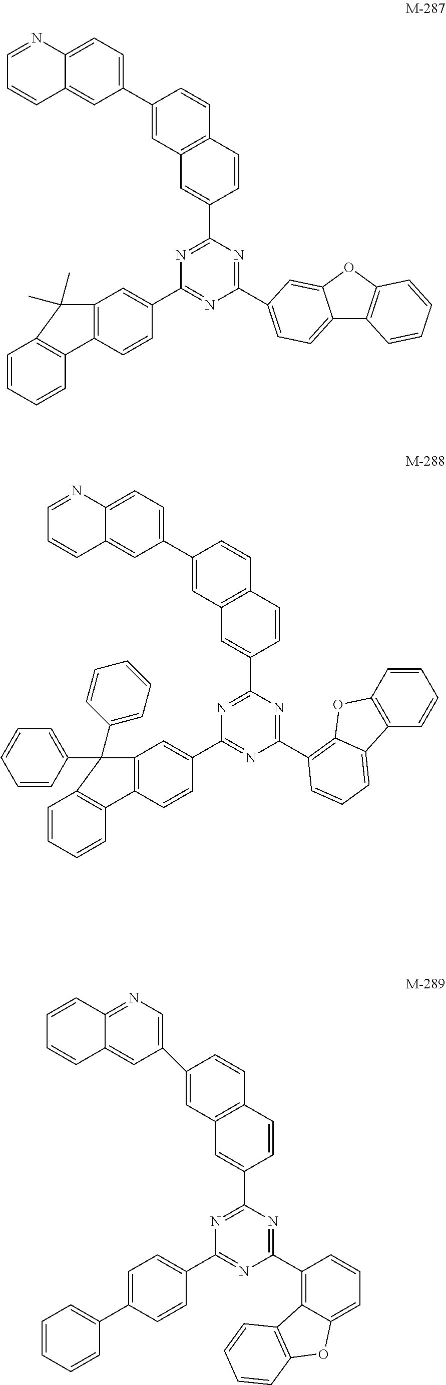 [SMILES: CC1(C)c2ccccc2-c2ccc(-c3nc(-c4ccc5ccc(-c6ccc7ncccc7c6)cc5c4)nc(-c4ccc5c(c4)oc4ccccc45)n3)cc21 ; c1ccc(C2(c3ccccc3)c3ccccc3-c3ccc(-c4nc(-c5ccc6ccc(-c7ccc8ncccc8c7)cc6c5)nc(-c5cccc6c5oc5ccccc56)n4)cc32)cc1 ; c1ccc(-c2ccc(-c3nc(-c4ccc5ccc(-c6cnc7ccccc7c6)cc5c4)nc(-c4cccc5oc6ccccc6c45)n3)cc2)cc1]

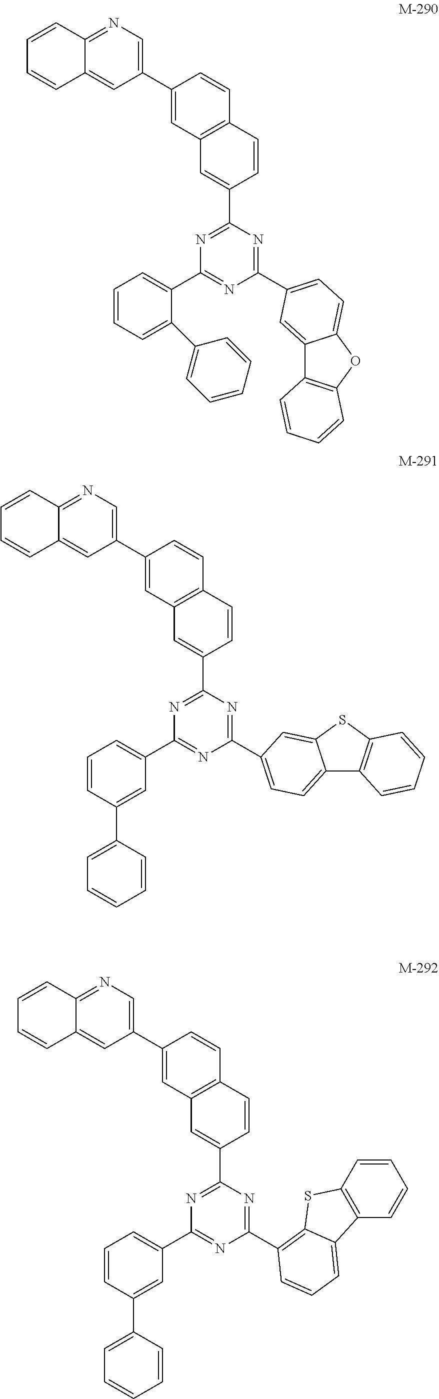 [SMILES: c1ccc(-c2ccccc2-c2nc(-c3ccc4ccc(-c5cnc6ccccc6c5)cc4c3)nc(-c3ccc4oc5ccccc5c4c3)n2)cc1 ; c1ccc(-c2cccc(-c3nc(-c4ccc5ccc(-c6cnc7ccccc7c6)cc5c4)nc(-c4ccc5c(c4)sc4ccccc45)n3)c2)cc1 ; c1ccc(-c2cccc(-c3nc(-c4ccc5ccc(-c6cnc7ccccc7c6)cc5c4)nc(-c4cccc5c4sc4ccccc45)n3)c2)cc1]

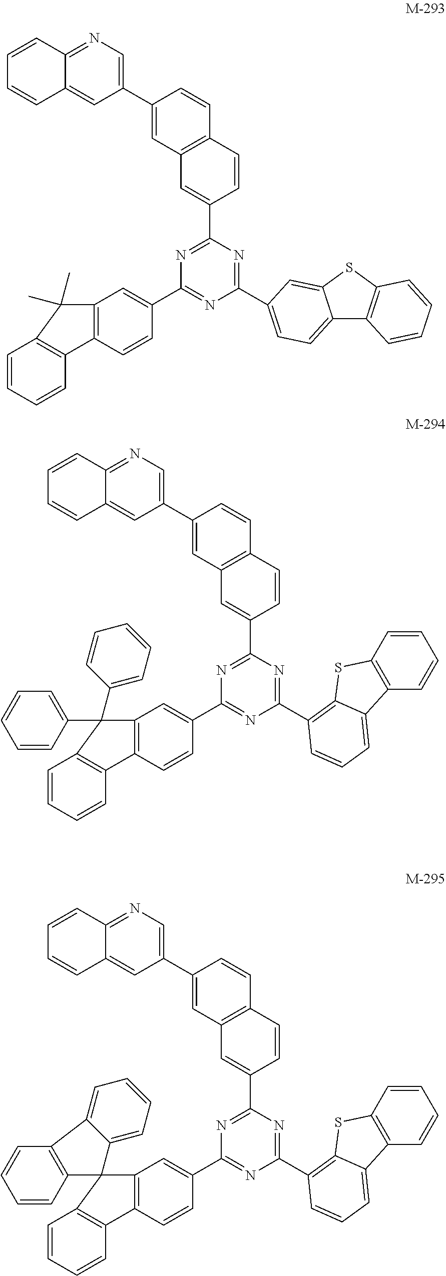 [SMILES: CC1(C)c2ccccc2-c2ccc(-c3nc(-c4ccc5ccc(-c6cnc7ccccc7c6)cc5c4)nc(-c4ccc5c(c4)sc4ccccc45)n3)cc21 ; c1ccc(C2(c3ccccc3)c3ccccc3-c3ccc(-c4nc(-c5ccc6ccc(-c7cnc8ccccc8c7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6cnc7ccccc7c6)cc5c4)nc(-c4cccc5c4sc4ccccc45)n3)cc21]

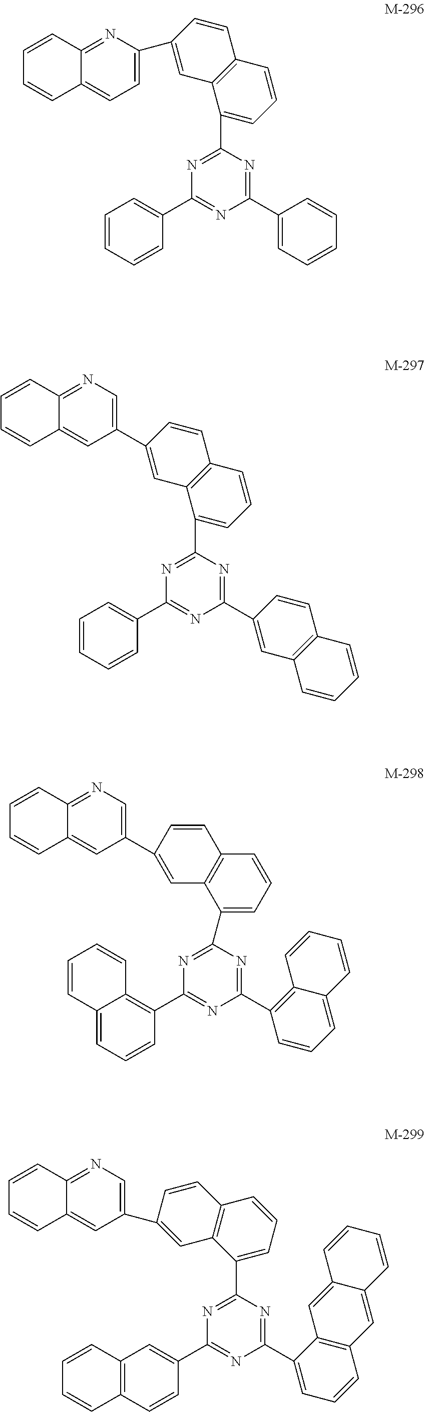 [SMILES: c1ccc(-c2nc(-c3ccccc3)nc(-c3cccc4ccc(-c5ccc6ccccc6n5)cc34)n2)cc1 ; c1ccc(-c2nc(-c3ccc4ccccc4c3)nc(-c3cccc4ccc(-c5cnc6ccccc6c5)cc34)n2)cc1 ; c1ccc2ncc(-c3ccc4cccc(-c5nc(-c6cccc7ccccc67)nc(-c6cccc7ccccc67)n5)c4c3)cc2c1 ; c1ccc2cc(-c3nc(-c4cccc5ccc(-c6cnc7ccccc7c6)cc45)nc(-c4cccc5cc6ccccc6cc45)n3)ccc2c1]

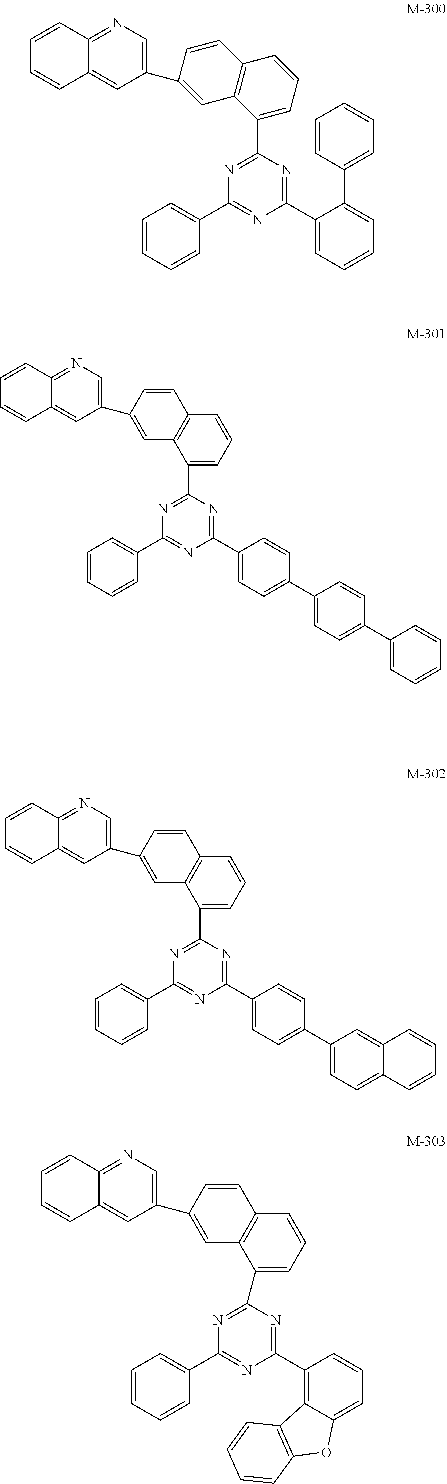 [SMILES: c1ccc(-c2nc(-c3ccccc3-c3ccccc3)nc(-c3cccc4ccc(-c5cnc6ccccc6c5)cc34)n2)cc1 ; c1ccc(-c2ccc(-c3ccc(-c4nc(-c5ccccc5)nc(-c5cccc6ccc(-c7cnc8ccccc8c7)cc56)n4)cc3)cc2)cc1 ; c1ccc(-c2nc(-c3ccc(-c4ccc5ccccc5c4)cc3)nc(-c3cccc4ccc(-c5cnc6ccccc6c5)cc34)n2)cc1 ; c1ccc(-c2nc(-c3cccc4ccc(-c5cnc6ccccc6c5)cc34)nc(-c3cccc4oc5ccccc5c34)n2)cc1]

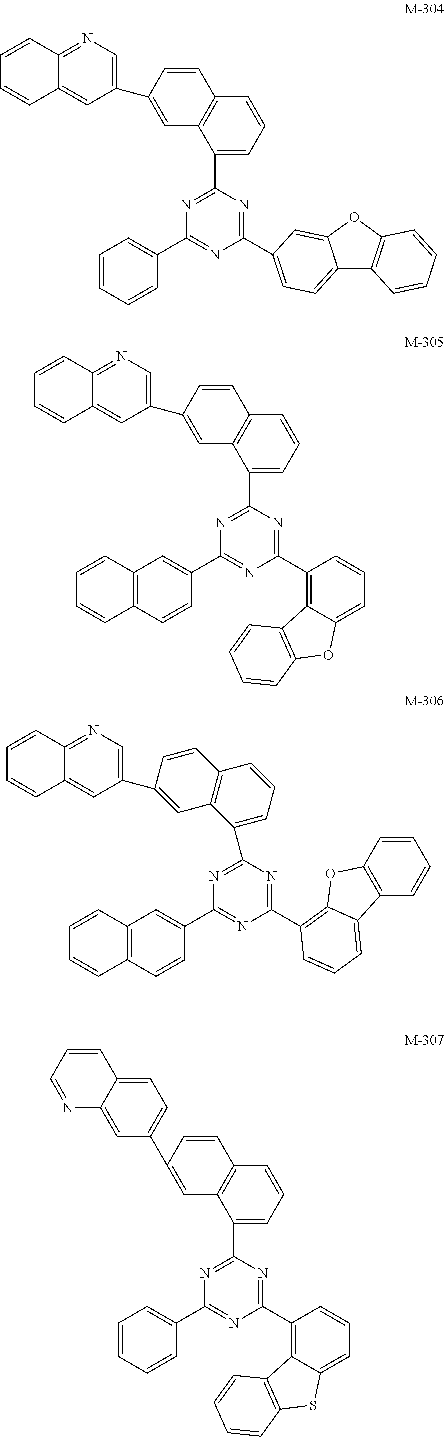 [SMILES: c1ccc(-c2nc(-c3ccc4c(c3)oc3ccccc34)nc(-c3cccc4ccc(-c5cnc6ccccc6c5)cc34)n2)cc1 ; c1ccc2cc(-c3nc(-c4cccc5ccc(-c6cnc7ccccc7c6)cc45)nc(-c4cccc5oc6ccccc6c45)n3)ccc2c1 ; c1ccc2cc(-c3nc(-c4cccc5ccc(-c6cnc7ccccc7c6)cc45)nc(-c4cccc5c4oc4ccccc45)n3)ccc2c1 ; c1ccc(-c2nc(-c3cccc4ccc(-c5ccc6cccnc6c5)cc34)nc(-c3cccc4sc5ccccc5c34)n2)cc1]

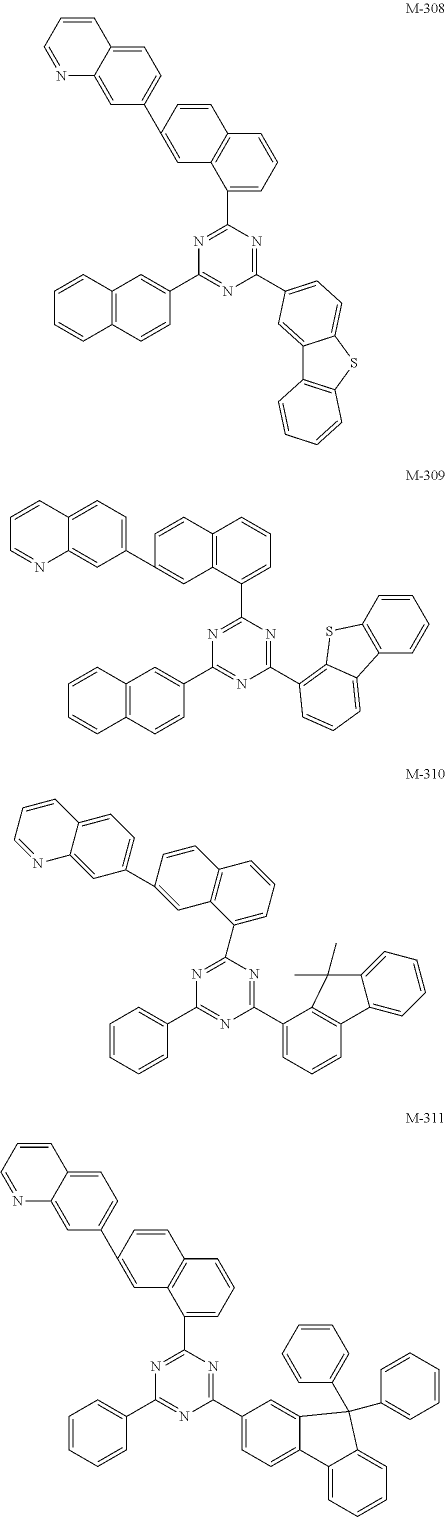 [SMILES: c1ccc2cc(-c3nc(-c4ccc5sc6ccccc6c5c4)nc(-c4cccc5ccc(-c6ccc7cccnc7c6)cc45)n3)ccc2c1 ; c1ccc2cc(-c3nc(-c4cccc5ccc(-c6ccc7cccnc7c6)cc45)nc(-c4cccc5c4sc4ccccc45)n3)ccc2c1 ; CC1(C)c2ccccc2-c2cccc(-c3nc(-c4ccccc4)nc(-c4cccc5ccc(-c6ccc7cccnc7c6)cc45)n3)c21 ; c1ccc(-c2nc(-c3ccc4c(c3)C(c3ccccc3)(c3ccccc3)c3ccccc3-4)nc(-c3cccc4ccc(-c5ccc6cccnc6c5)cc34)n2)cc1]

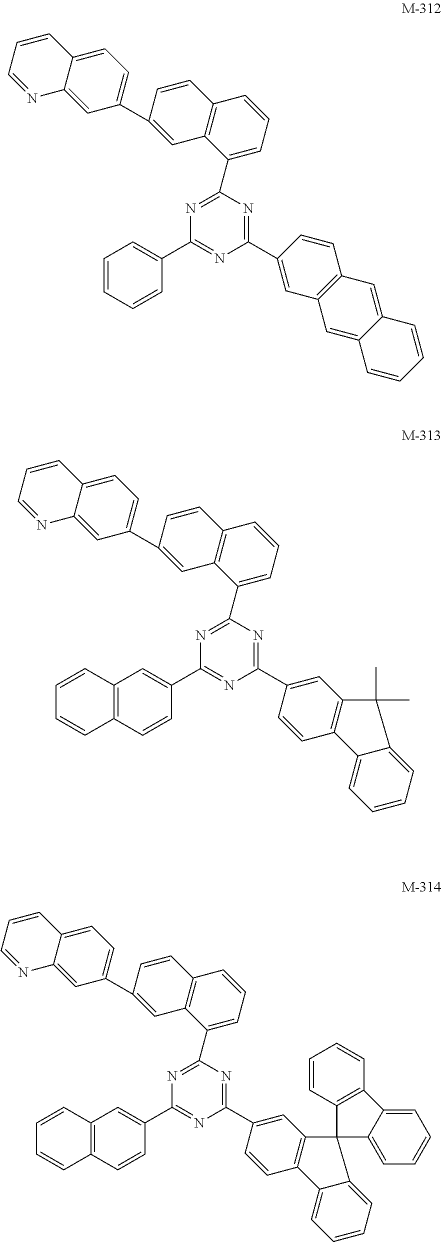 [SMILES: c1ccc(-c2nc(-c3ccc4cc5ccccc5cc4c3)nc(-c3cccc4ccc(-c5ccc6cccnc6c5)cc34)n2)cc1 ; CC1(C)c2ccccc2-c2ccc(-c3nc(-c4ccc5ccccc5c4)nc(-c4cccc5ccc(-c6ccc7cccnc7c6)cc45)n3)cc21 ; c1ccc2c(c1)-c1ccccc1C21c2ccccc2-c2ccc(-c3nc(-c4ccc5ccccc5c4)nc(-c4cccc5ccc(-c6ccc7cccnc7c6)cc45)n3)cc21]

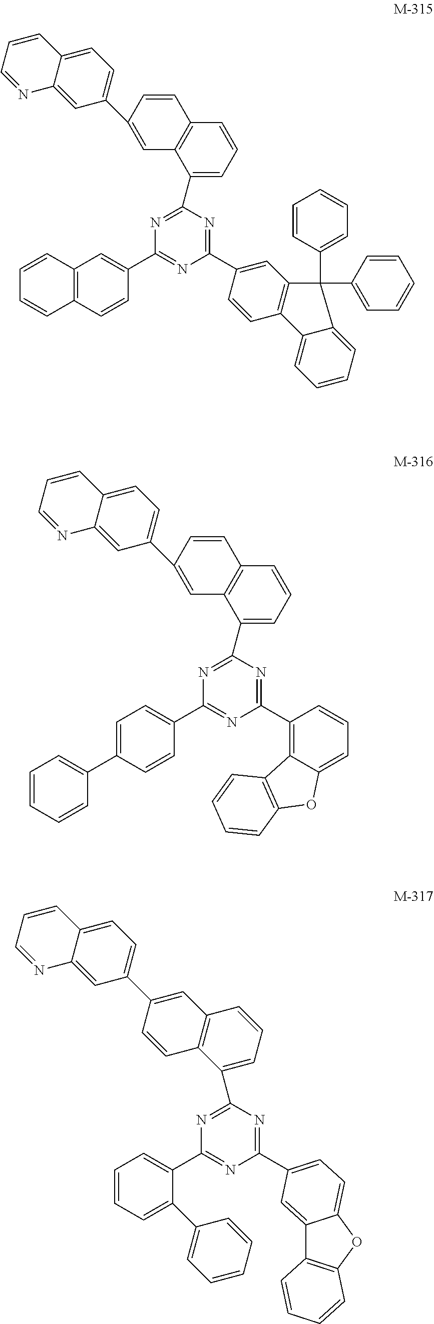 [SMILES: c1ccc(C2(c3ccccc3)c3ccccc3-c3ccc(-c4nc(-c5ccc6ccccc6c5)nc(-c5cccc6ccc(-c7ccc8cccnc8c7)cc56)n4)cc32)cc1 ; c1ccc(-c2ccc(-c3nc(-c4cccc5ccc(-c6ccc7cccnc7c6)cc45)nc(-c4cccc5oc6ccccc6c45)n3)cc2)cc1 ; c1ccc(-c2ccccc2-c2nc(-c3ccc4oc5ccccc5c4c3)nc(-c3cccc4cc(-c5ccc6cccnc6c5)ccc34)n2)cc1]

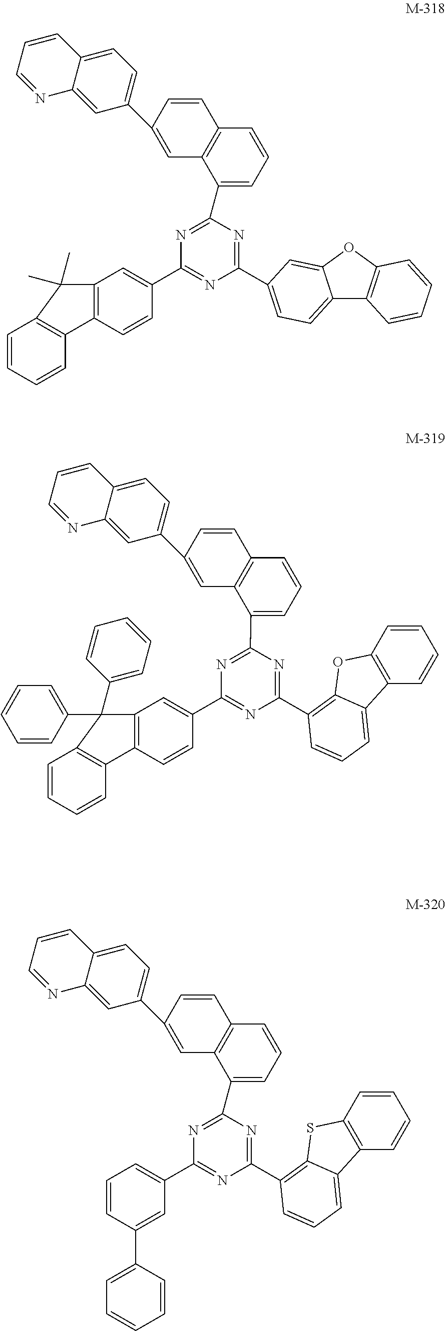 [SMILES: CC1(C)c2ccccc2-c2ccc(-c3nc(-c4ccc5c(c4)oc4ccccc45)nc(-c4cccc5ccc(-c6ccc7cccnc7c6)cc45)n3)cc21 ; c1ccc(C2(c3ccccc3)c3ccccc3-c3ccc(-c4nc(-c5cccc6ccc(-c7ccc8cccnc8c7)cc56)nc(-c5cccc6c5oc5ccccc56)n4)cc32)cc1 ; c1ccc(-c2cccc(-c3nc(-c4cccc5ccc(-c6ccc7cccnc7c6)cc45)nc(-c4cccc5c4sc4ccccc45)n3)c2)cc1]

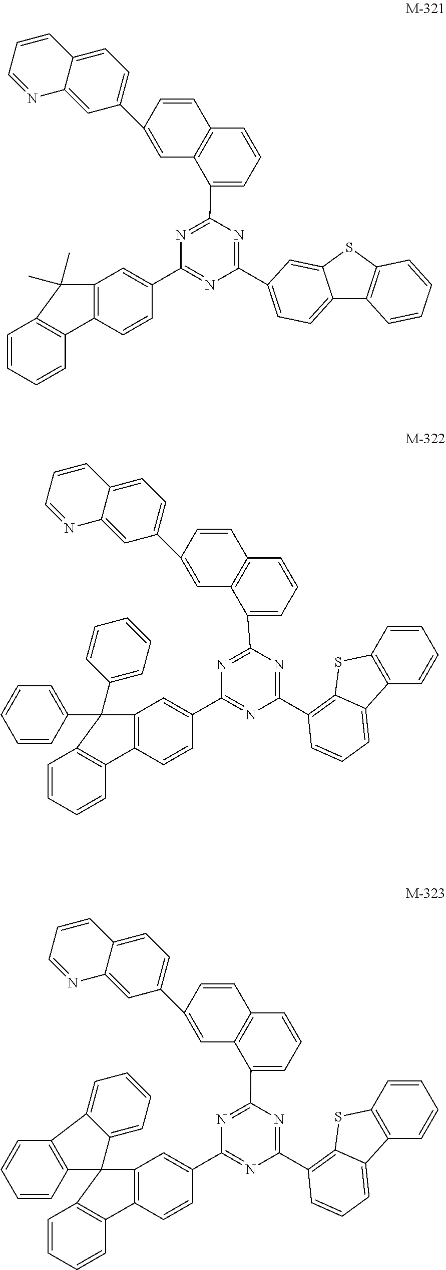 [SMILES: CC1(C)c2ccccc2-c2ccc(-c3nc(-c4ccc5c(c4)sc4ccccc45)nc(-c4cccc5ccc(-c6ccc7cccnc7c6)cc45)n3)cc21 ; c1ccc(C2(c3ccccc3)c3ccccc3-c3ccc(-c4nc(-c5cccc6ccc(-c7ccc8cccnc8c7)cc56)nc(-c5cccc6c5sc5ccccc56)n4)cc32)cc1 ; c1ccc2c(c1)-c1ccccc1C21c2ccccc2-c2ccc(-c3nc(-c4cccc5ccc(-c6ccc7cccnc7c6)cc45)nc(-c4cccc5c4sc4ccccc45)n3)cc21]

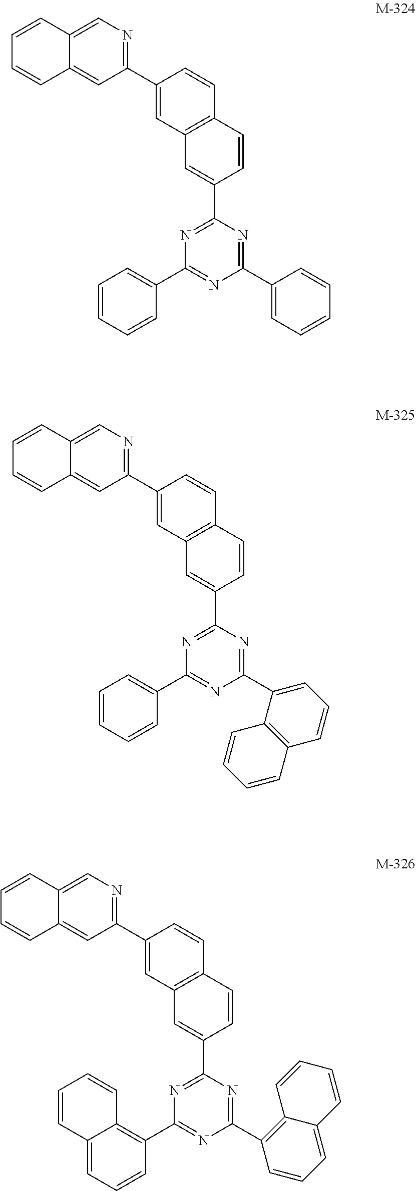 [SMILES: c1ccc(-c2nc(-c3ccccc3)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3cccc4ccccc34)n2)cc1 ; c1ccc2cc(-c3ccc4ccc(-c5nc(-c6cccc7ccccc67)nc(-c6cccc7ccccc67)n5)cc4c3)ncc2c1]

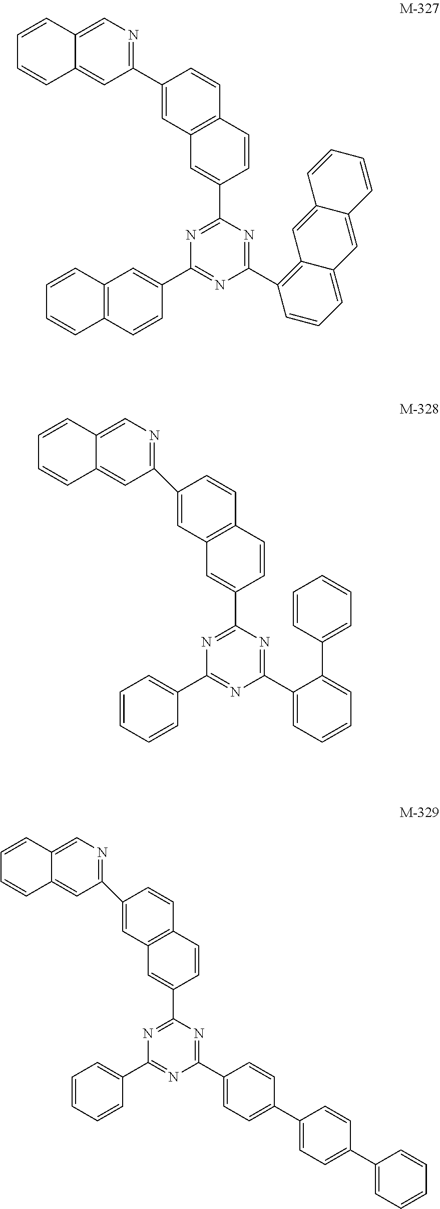 [SMILES: c1ccc2cc(-c3nc(-c4ccc5ccc(-c6cc7ccccc7cn6)cc5c4)nc(-c4cccc5cc6ccccc6cc45)n3)ccc2c1 ; c1ccc(-c2nc(-c3ccc4ccc(-c5cc6ccccc6cn5)cc4c3)nc(-c3ccccc3-c3ccccc3)n2)cc1 ; c1ccc(-c2ccc(-c3ccc(-c4nc(-c5ccccc5)nc(-c5ccc6ccc(-c7cc8ccccc8cn7)cc6c5)n4)cc3)cc2)cc1]

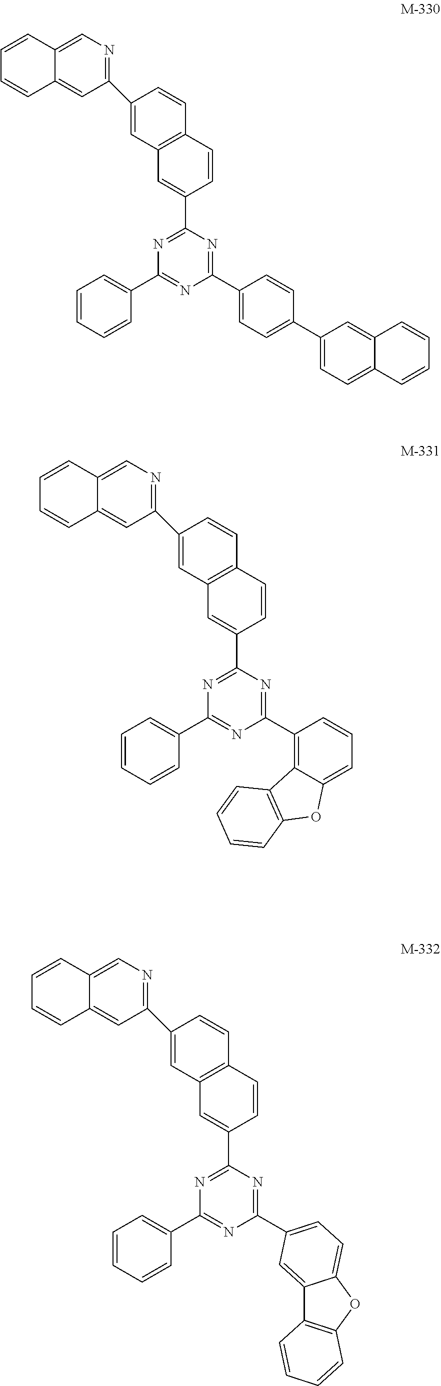 [SMILES: c1ccc(-c2nc(-c3ccc(-c4ccc5ccccc5c4)cc3)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3cccc4oc5ccccc5c34)n2)cc1 ; c1ccc(-c2nc(-c3ccc4ccc(-c5cc6ccccc6cn5)cc4c3)nc(-c3ccc4oc5ccccc5c4c3)n2)cc1]

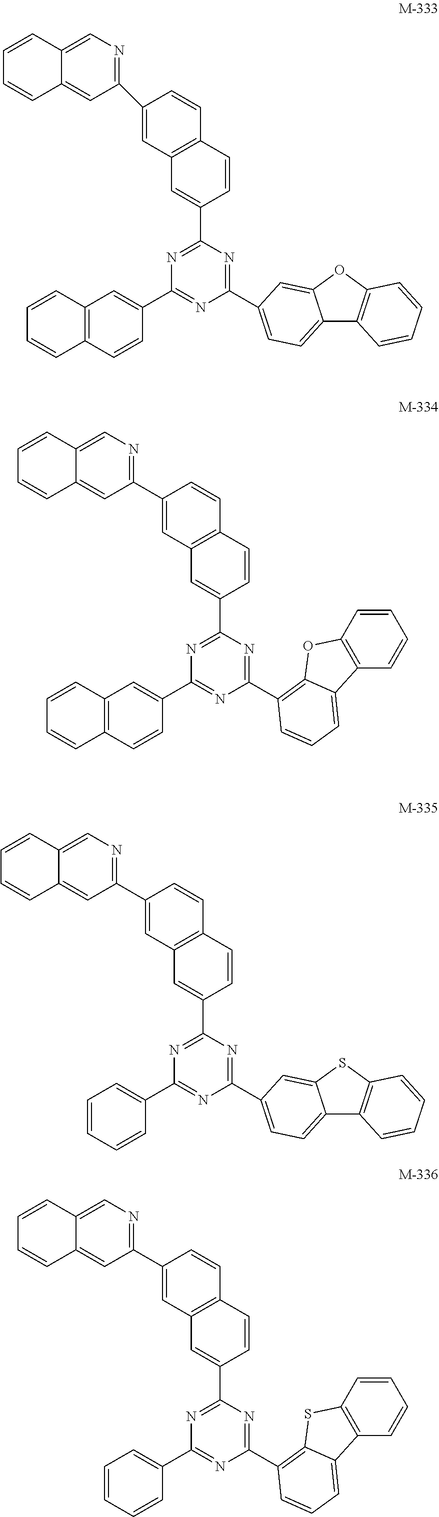 [SMILES: c1ccc2cc(-c3nc(-c4ccc5ccc(-c6cc7ccccc7cn6)cc5c4)nc(-c4ccc5c(c4)oc4ccccc45)n3)ccc2c1 ; c1ccc2cc(-c3nc(-c4ccc5ccc(-c6cc7ccccc7cn6)cc5c4)nc(-c4cccc5c4oc4ccccc45)n3)ccc2c1 ; c1ccc(-c2nc(-c3ccc4ccc(-c5cc6ccccc6cn5)cc4c3)nc(-c3ccc4c(c3)sc3ccccc34)n2)cc1 ; c1ccc(-c2nc(-c3ccc4ccc(-c5cc6ccccc6cn5)cc4c3)nc(-c3cccc4c3sc3ccccc34)n2)cc1]

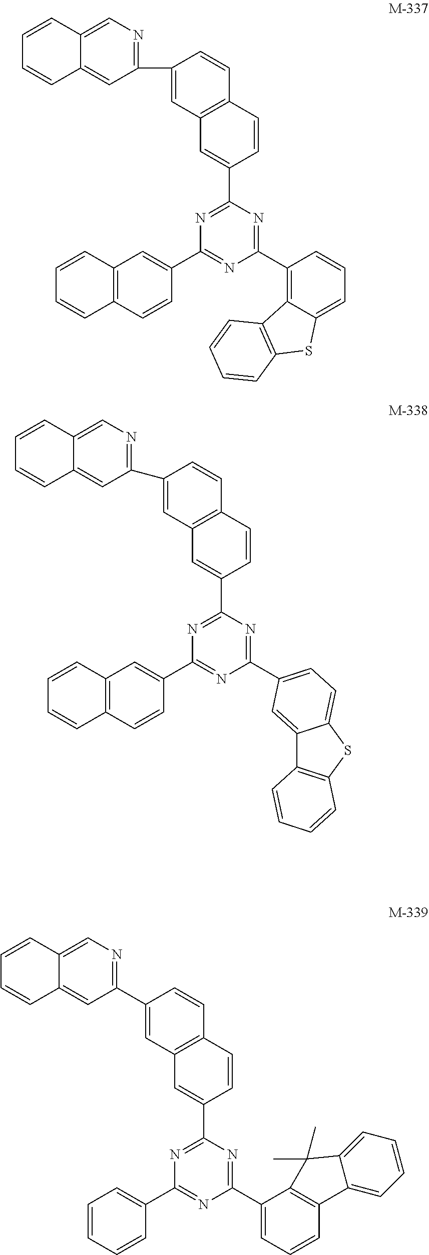 [SMILES: c1ccc2cc(-c3nc(-c4ccc5ccc(-c6cc7ccccc7cn6)cc5c4)nc(-c4cccc5sc6ccccc6c45)n3)ccc2c1 ; c1ccc2cc(-c3nc(-c4ccc5ccc(-c6cc7ccccc7cn6)cc5c4)nc(-c4ccc5sc6ccccc6c5c4)n3)ccc2c1 ; CC1(C)c2ccccc2-c2cccc(-c3nc(-c4ccccc4)nc(-c4ccc5ccc(-c6cc7ccccc7cn6)cc5c4)n3)c21]

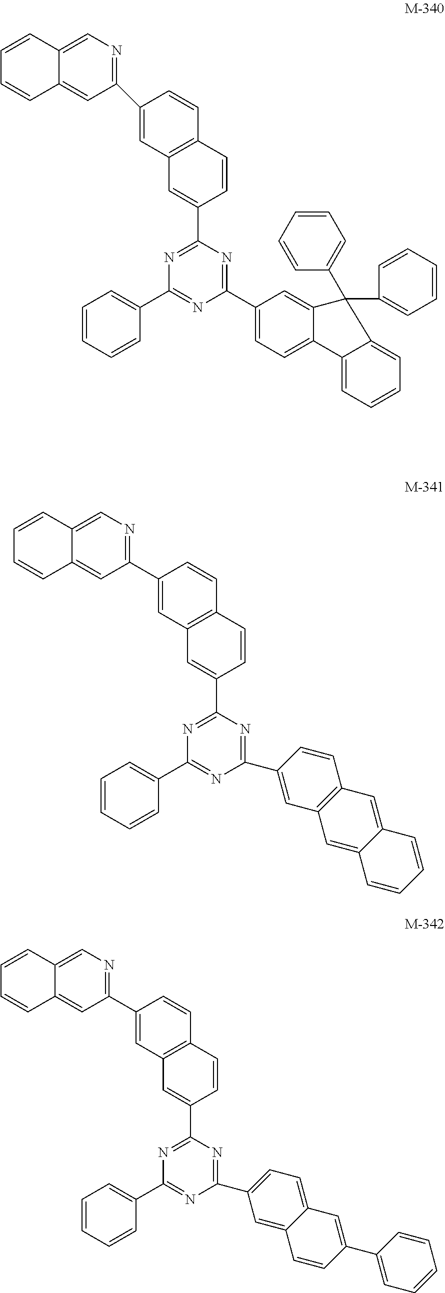 [SMILES: c1ccc(-c2nc(-c3ccc4c(c3)C(c3ccccc3)(c3ccccc3)c3ccccc3-4)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3ccc4cc5ccccc5cc4c3)n2)cc1 ; c1ccc(-c2ccc3cc(-c4nc(-c5ccccc5)nc(-c5ccc6ccc(-c7cc8ccccc8cn7)cc6c5)n4)ccc3c2)cc1]

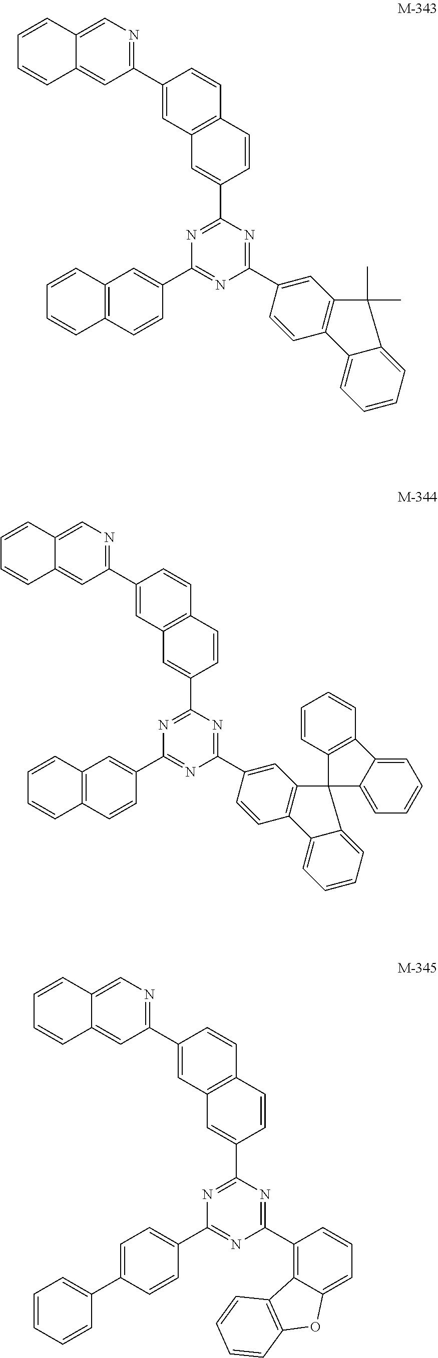 [SMILES: CC1(C)c2ccccc2-c2ccc(-c3nc(-c4ccc5ccccc5c4)nc(-c4ccc5ccc(-c6cc7ccccc7cn6)cc5c4)n3)cc21 ; c1ccc2c(c1)-c1ccccc1C21c2ccccc2-c2ccc(-c3nc(-c4ccc5ccccc5c4)nc(-c4ccc5ccc(-c6cc7ccccc7cn6)cc5c4)n3)cc21 ; c1ccc(-c2ccc(-c3nc(-c4ccc5ccc(-c6cc7ccccc7cn6)cc5c4)nc(-c4cccc5oc6ccccc6c45)n3)cc2)cc1]

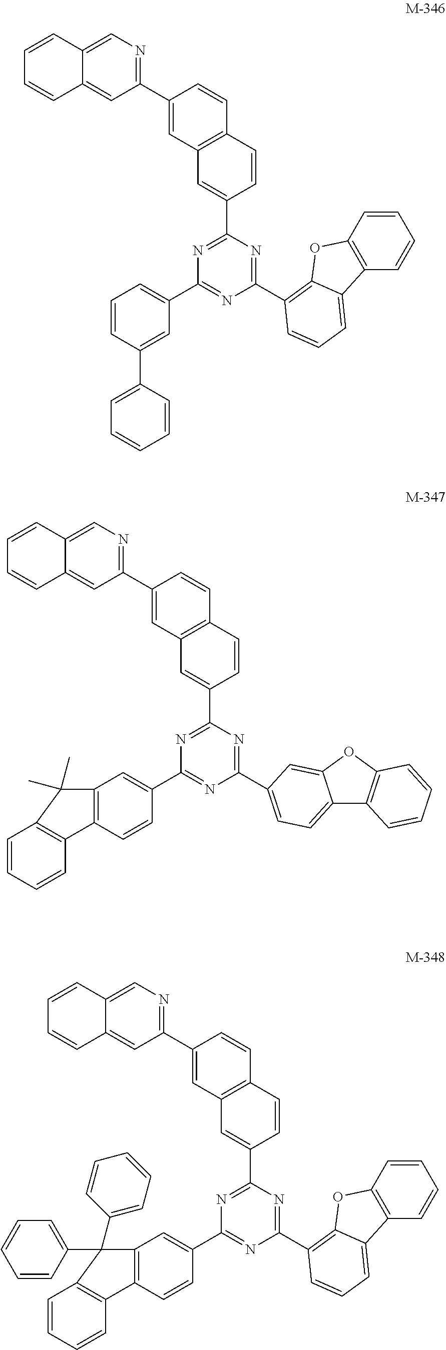 [SMILES: c1ccc(-c2cccc(-c3nc(-c4ccc5ccc(-c6cc7ccccc7cn6)cc5c4)nc(-c4cccc5c4oc4ccccc45)n3)c2)cc1 ; CC1(C)c2ccccc2-c2ccc(-c3nc(-c4ccc5ccc(-c6cc7ccccc7cn6)cc5c4)nc(-c4ccc5c(c4)oc4ccccc45)n3)cc21 ; c1ccc(C2(c3ccccc3)c3ccccc3-c3ccc(-c4nc(-c5ccc6ccc(-c7cc8ccccc8cn7)cc6c5)nc(-c5cccc6c5oc5ccccc56)n4)cc32)cc1]

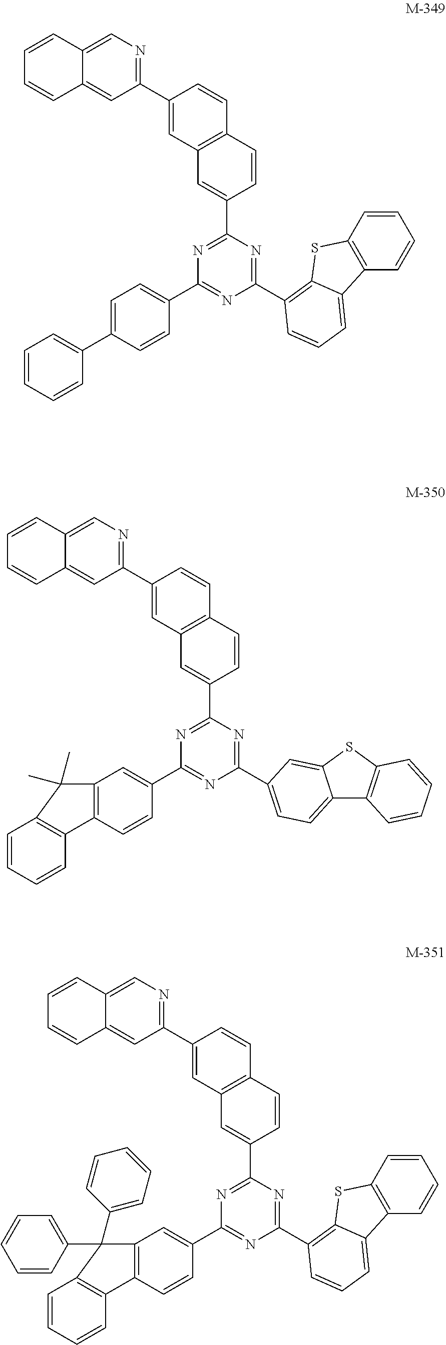 [SMILES: c1ccc(-c2ccc(-c3nc(-c4ccc5ccc(-c6cc7ccccc7cn6)cc5c4)nc(-c4cccc5c4sc4ccccc45)n3)cc2)cc1 ; CC1(C)c2ccccc2-c2ccc(-c3nc(-c4ccc5ccc(-c6cc7ccccc7cn6)cc5c4)nc(-c4ccc5c(c4)sc4ccccc45)n3)cc21 ; c1ccc(C2(c3ccccc3)c3ccccc3-c3ccc(-c4nc(-c5ccc6ccc(-c7cc8ccccc8cn7)cc6c5)nc(-c5cccc6c5sc5ccccc56)n4)cc32)cc1]

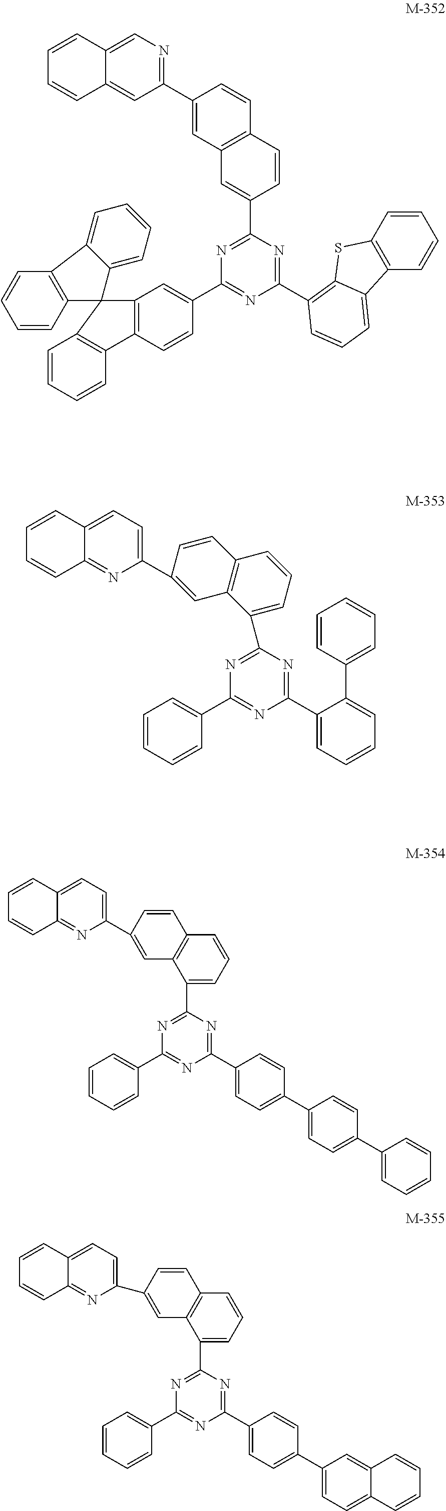 [SMILES: c1ccc2c(c1)-c1ccccc1C21c2ccccc2-c2ccc(-c3nc(-c4ccc5ccc(-c6cc7ccccc7cn6)cc5c4)nc(-c4cccc5c4sc4ccccc45)n3)cc21 ; c1ccc(-c2nc(-c3ccccc3-c3ccccc3)nc(-c3cccc4ccc(-c5ccc6ccccc6n5)cc34)n2)cc1 ; c1ccc(-c2ccc(-c3ccc(-c4nc(-c5ccccc5)nc(-c5cccc6ccc(-c7ccc8ccccc8n7)cc56)n4)cc3)cc2)cc1 ; c1ccc(-c2nc(-c3ccc(-c4ccc5ccccc5c4)cc3)nc(-c3cccc4ccc(-c5ccc6ccccc6n5)cc34)n2)cc1]

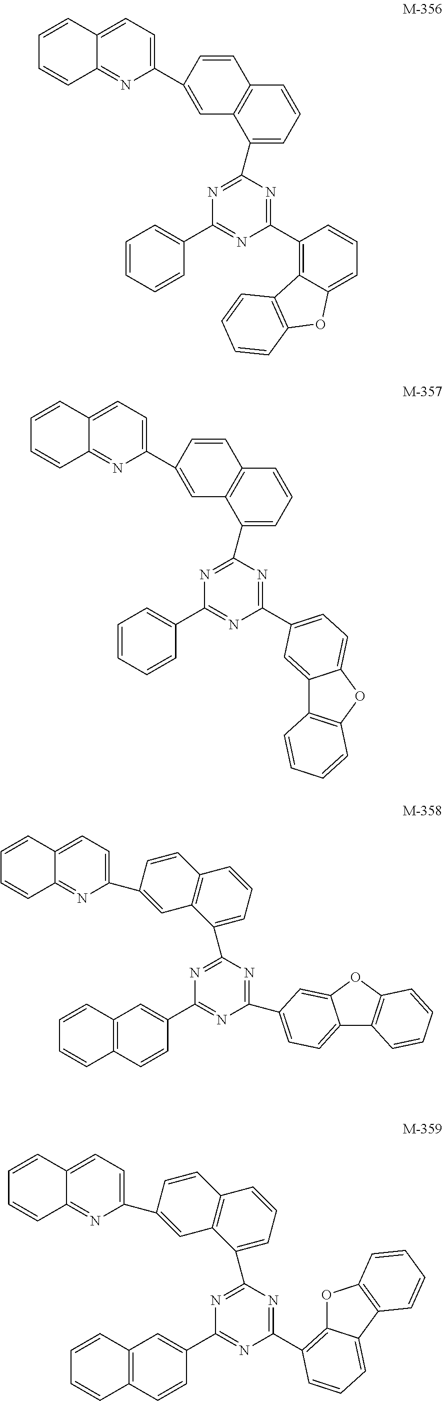 [SMILES: c1ccc(-c2nc(-c3cccc4ccc(-c5ccc6ccccc6n5)cc34)nc(-c3cccc4oc5ccccc5c34)n2)cc1 ; c1ccc(-c2nc(-c3ccc4oc5ccccc5c4c3)nc(-c3cccc4ccc(-c5ccc6ccccc6n5)cc34)n2)cc1 ; c1ccc2cc(-c3nc(-c4ccc5c(c4)oc4ccccc45)nc(-c4cccc5ccc(-c6ccc7ccccc7n6)cc45)n3)ccc2c1 ; c1ccc2cc(-c3nc(-c4cccc5ccc(-c6ccc7ccccc7n6)cc45)nc(-c4cccc5c4oc4ccccc45)n3)ccc2c1]

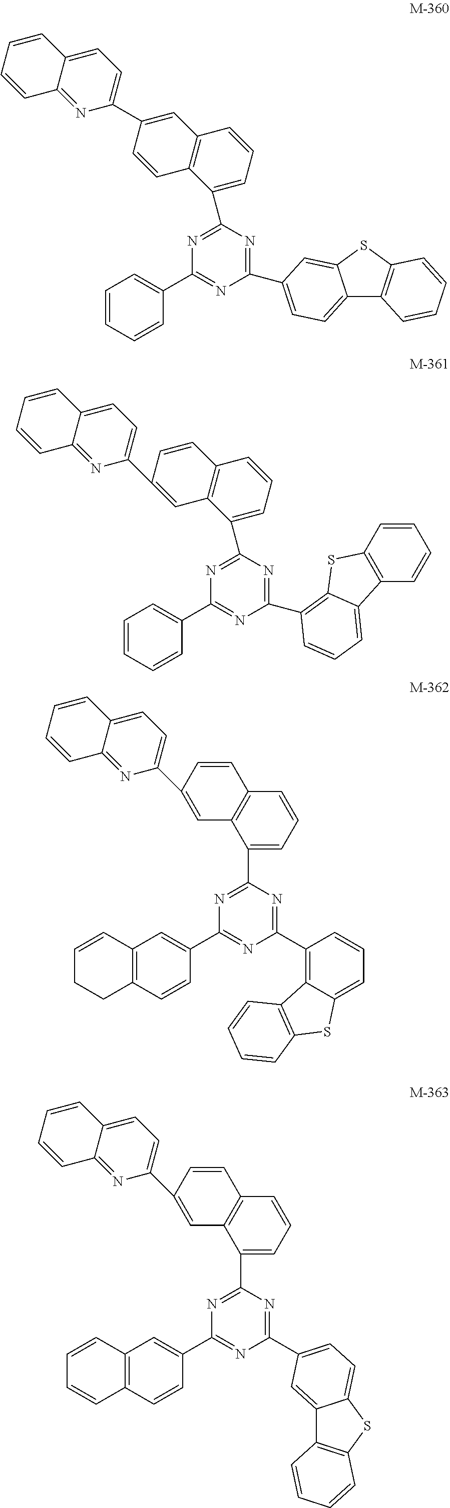

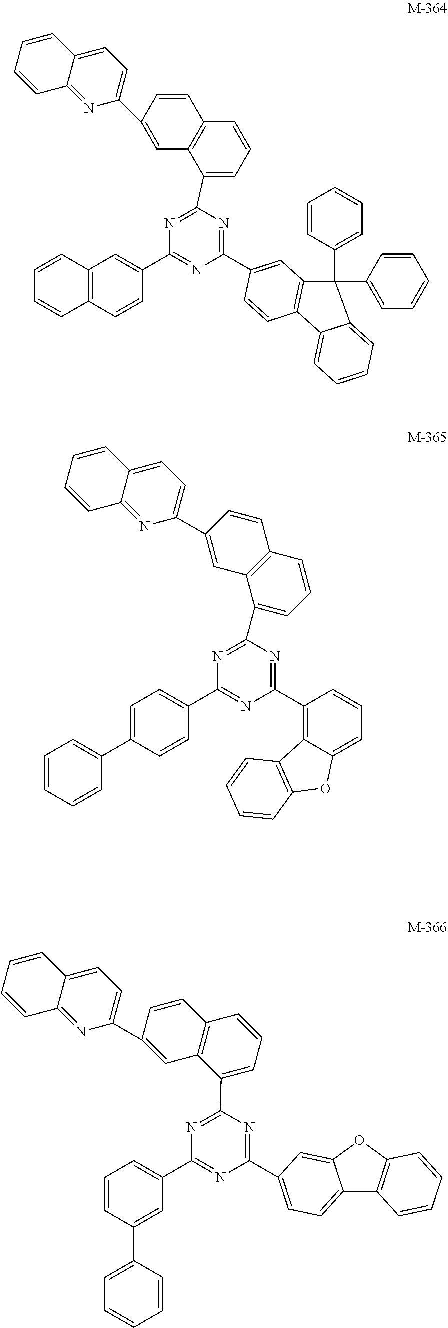 [SMILES: c1ccc(C2(c3ccccc3)c3ccccc3-c3ccc(-c4nc(-c5ccc6ccccc6c5)nc(-c5cccc6ccc(-c7ccc8ccccc8n7)cc56)n4)cc32)cc1 ; c1ccc(-c2ccc(-c3nc(-c4cccc5ccc(-c6ccc7ccccc7n6)cc45)nc(-c4cccc5oc6ccccc6c45)n3)cc2)cc1 ; c1ccc(-c2cccc(-c3nc(-c4ccc5c(c4)oc4ccccc45)nc(-c4cccc5ccc(-c6ccc7ccccc7n6)cc45)n3)c2)cc1]

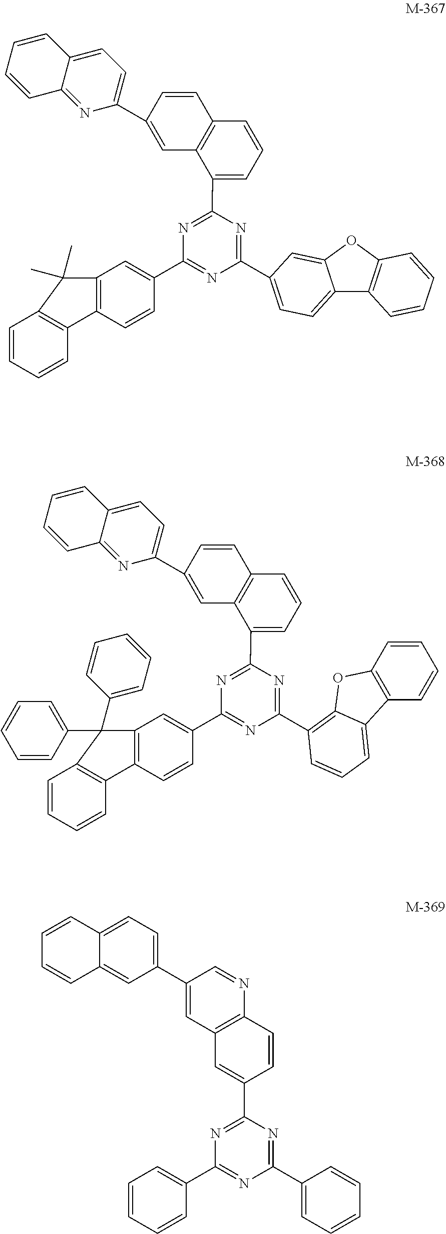 [SMILES: CC1(C)c2ccccc2-c2ccc(-c3nc(-c4ccc5c(c4)oc4ccccc45)nc(-c4cccc5ccc(-c6ccc7ccccc7n6)cc45)n3)cc21 ; c1ccc(C2(c3ccccc3)c3ccccc3-c3ccc(-c4nc(-c5cccc6ccc(-c7ccc8ccccc8n7)cc56)nc(-c5cccc6c5oc5ccccc56)n4)cc32)cc1 ; c1ccc(-c2nc(-c3ccccc3)nc(-c3ccc4ncc(-c5ccc6ccccc6c5)cc4c3)n2)cc1]

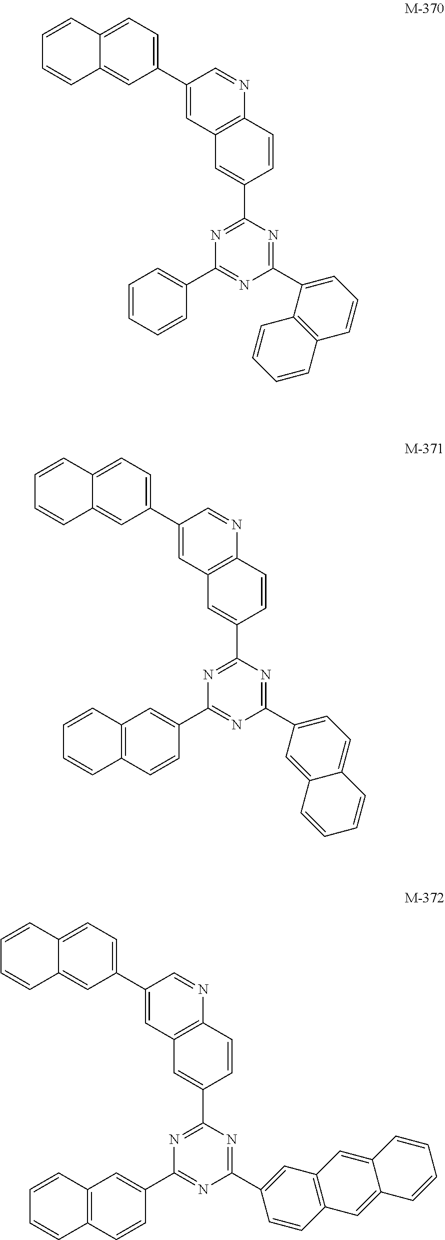 [SMILES: c1ccc(-c2nc(-c3ccc4ncc(-c5ccc6ccccc6c5)cc4c3)nc(-c3cccc4ccccc34)n2)cc1 ; c1ccc2cc(-c3cnc4ccc(-c5nc(-c6ccc7ccccc7c6)nc(-c6ccc7ccccc7c6)n5)cc4c3)ccc2c1 ; c1ccc2cc(-c3cnc4ccc(-c5nc(-c6ccc7ccccc7c6)nc(-c6ccc7cc8ccccc8cc7c6)n5)cc4c3)ccc2c1]

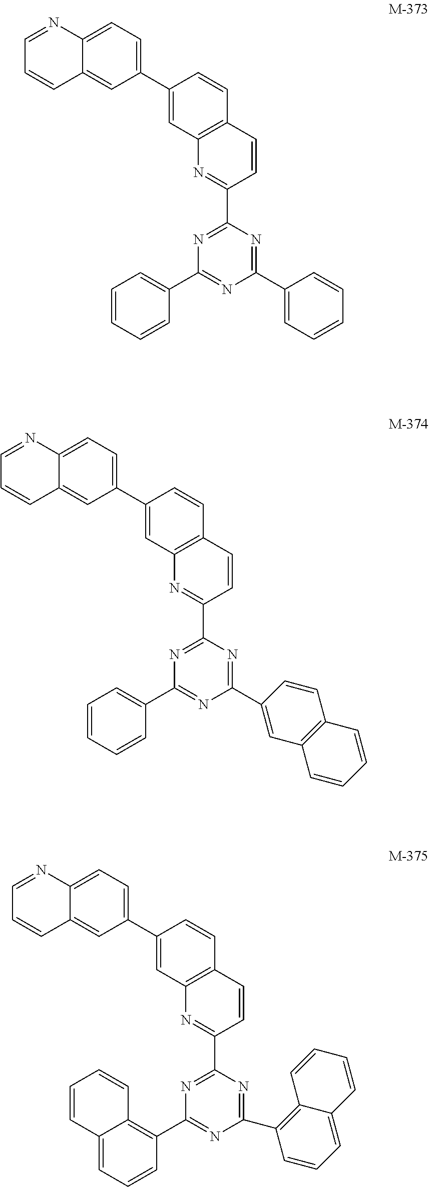 [SMILES: c1ccc(-c2nc(-c3ccccc3)nc(-c3ccc4ccc(-c5ccc6ncccc6c5)cc4n3)n2)cc1 ; c1ccc(-c2nc(-c3ccc4ccccc4c3)nc(-c3ccc4ccc(-c5ccc6ncccc6c5)cc4n3)n2)cc1 ; c1cnc2ccc(-c3ccc4ccc(-c5nc(-c6cccc7ccccc67)nc(-c6cccc7ccccc67)n5)nc4c3)cc2c1]

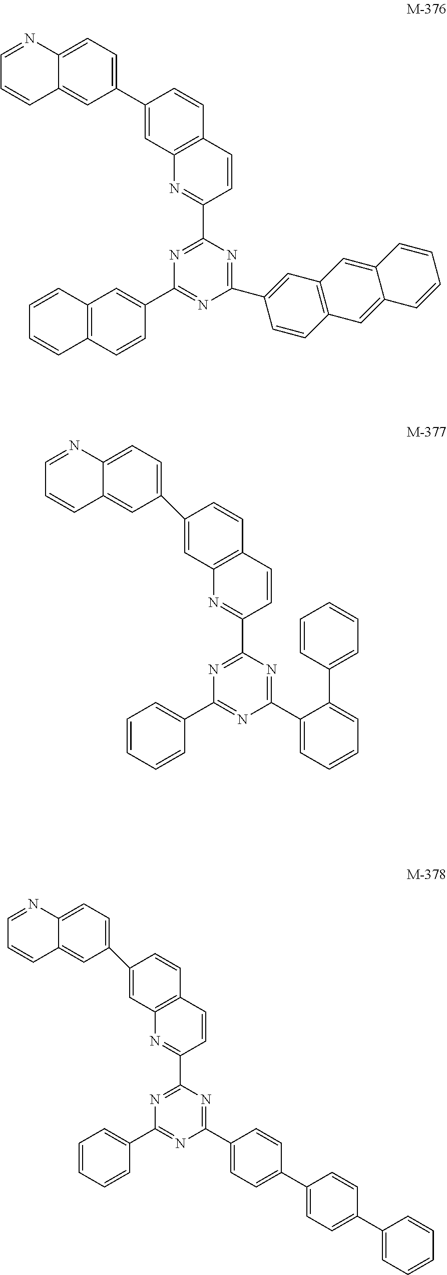 [SMILES: c1ccc2cc(-c3nc(-c4ccc5cc6ccccc6cc5c4)nc(-c4ccc5ccc(-c6ccc7ncccc7c6)cc5n4)n3)ccc2c1 ; c1ccc(-c2nc(-c3ccc4ccc(-c5ccc6ncccc6c5)cc4n3)nc(-c3ccccc3-c3ccccc3)n2)cc1 ; c1ccc(-c2ccc(-c3ccc(-c4nc(-c5ccccc5)nc(-c5ccc6ccc(-c7ccc8ncccc8c7)cc6n5)n4)cc3)cc2)cc1]

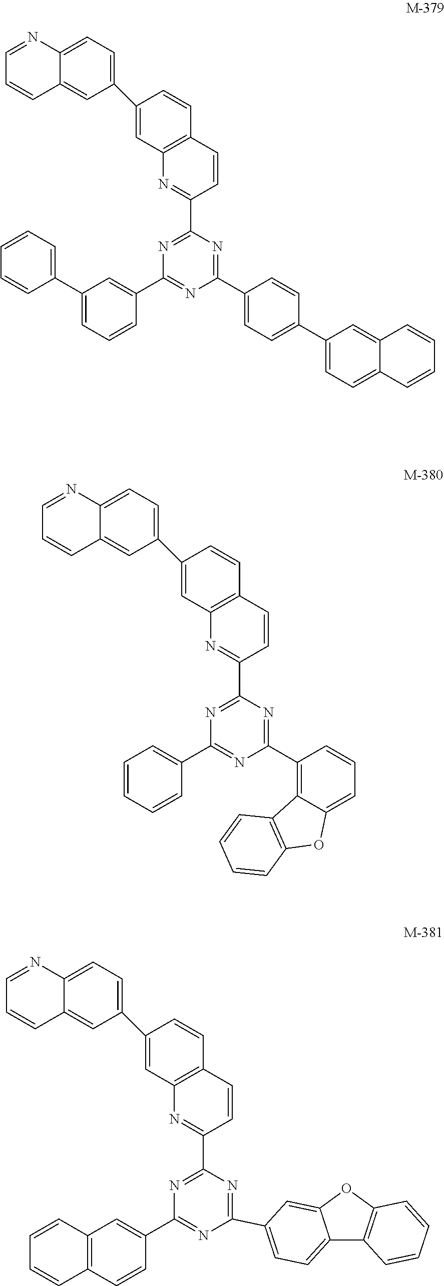 [SMILES: c1ccc(-c2cccc(-c3nc(-c4ccc(-c5ccc6ccccc6c5)cc4)nc(-c4ccc5ccc(-c6ccc7ncccc7c6)cc5n4)n3)c2)cc1 ; c1ccc(-c2nc(-c3ccc4ccc(-c5ccc6ncccc6c5)cc4n3)nc(-c3cccc4oc5ccccc5c34)n2)cc1 ; c1ccc2cc(-c3nc(-c4ccc5c(c4)oc4ccccc45)nc(-c4ccc5ccc(-c6ccc7ncccc7c6)cc5n4)n3)ccc2c1]

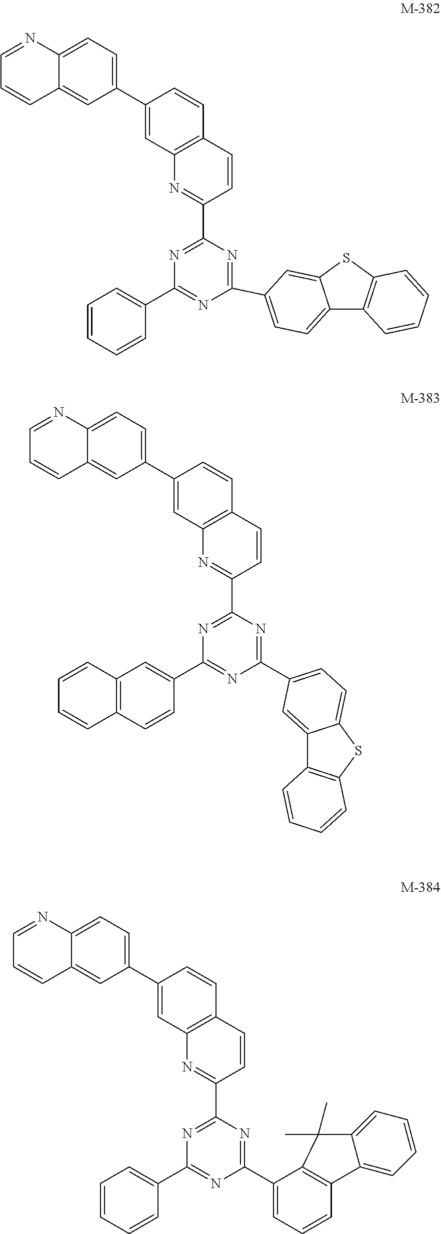 [SMILES: c1ccc(-c2nc(-c3ccc4c(c3)sc3ccccc34)nc(-c3ccc4ccc(-c5ccc6ncccc6c5)cc4n3)n2)cc1 ; c1ccc2cc(-c3nc(-c4ccc5sc6ccccc6c5c4)nc(-c4ccc5ccc(-c6ccc7ncccc7c6)cc5n4)n3)ccc2c1 ; CC1(C)c2ccccc2-c2cccc(-c3nc(-c4ccccc4)nc(-c4ccc5ccc(-c6ccc7ncccc7c6)cc5n4)n3)c21]

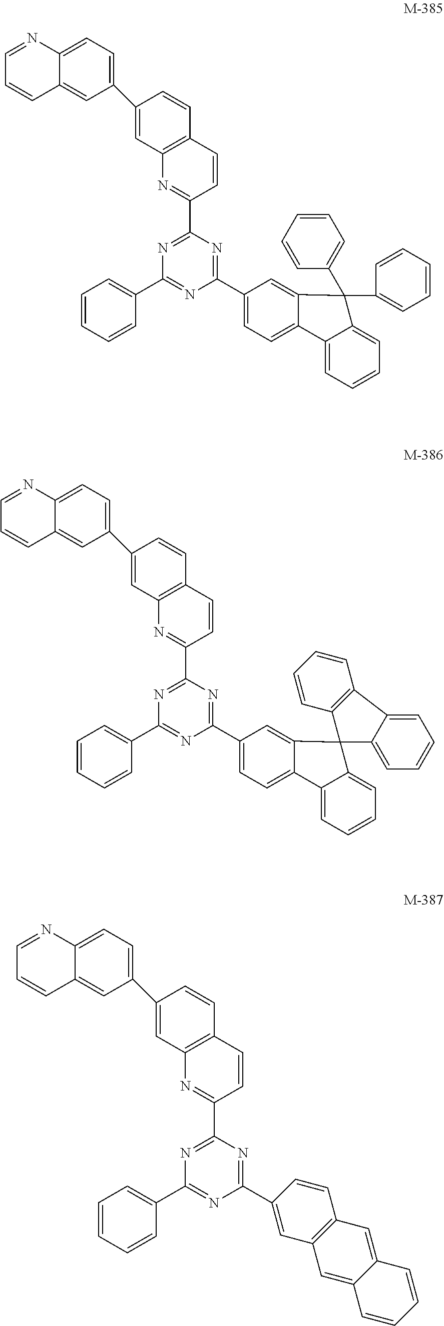 [SMILES: c1ccc(-c2nc(-c3ccc4c(c3)C(c3ccccc3)(c3ccccc3)c3ccccc3-4)nc(-c3ccc4ccc(-c5ccc6ncccc6c5)cc4n3)n2)cc1 ; c1ccc(-c2nc(-c3ccc4c(c3)C3(c5ccccc5-c5ccccc53)c3ccccc3-4)nc(-c3ccc4ccc(-c5ccc6ncccc6c5)cc4n3)n2)cc1 ; c1ccc(-c2nc(-c3ccc4cc5ccccc5cc4c3)nc(-c3ccc4ccc(-c5ccc6ncccc6c5)cc4n3)n2)cc1]

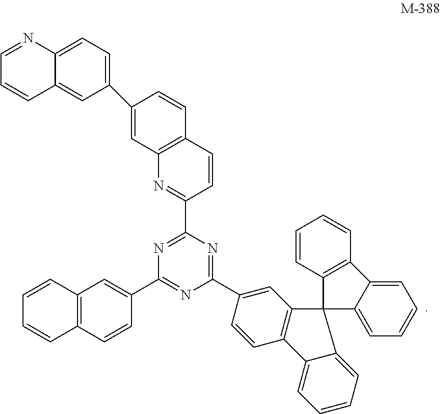 [SMILES: c1ccc2c(c1)-c1ccccc1C21c2ccccc2-c2ccc(-c3nc(-c4ccc5ccccc5c4)nc(-c4ccc5ccc(-c6ccc7ncccc7c6)cc5n4)n3)cc21]

7. The organic electroluminescent compound according to claim 1, wherein the structure of the compound M is as shown in any one of M-389 to M-619 below:

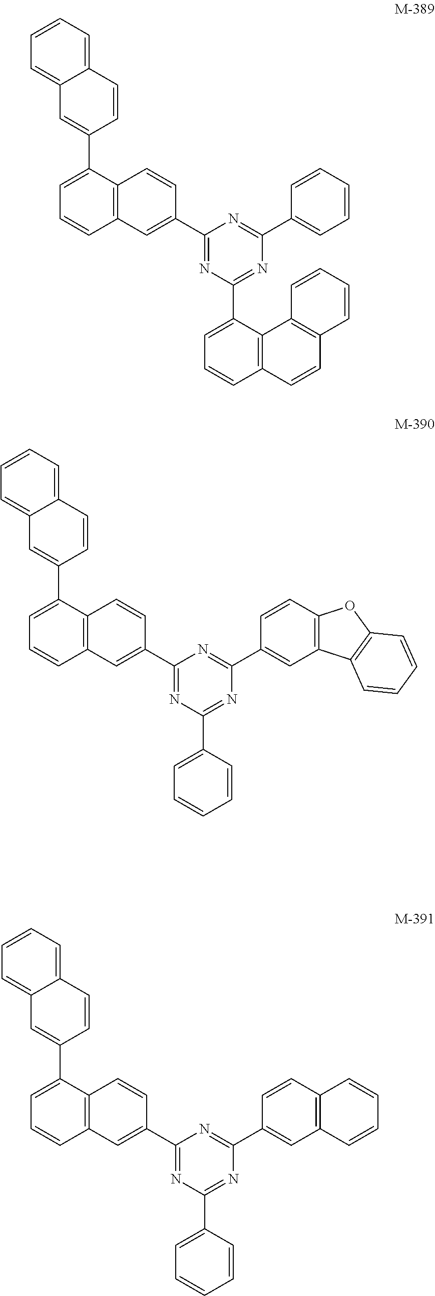 [SMILES: c1ccc(-c2nc(-c3ccc4c(-c5ccc6ccccc6c5)cccc4c3)nc(-c3cccc4ccc5ccccc5c34)n2)cc1 ; c1ccc(-c2nc(-c3ccc4c(-c5ccc6ccccc6c5)cccc4c3)nc(-c3ccc4oc5ccccc5c4c3)n2)cc1 ; c1ccc(-c2nc(-c3ccc4ccccc4c3)nc(-c3ccc4c(-c5ccc6ccccc6c5)cccc4c3)n2)cc1]

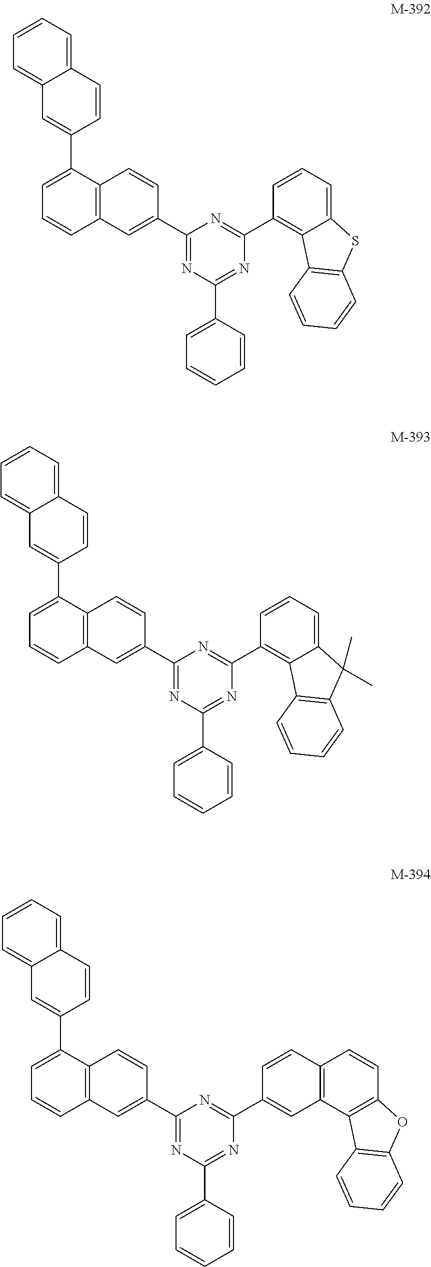 [SMILES: c1ccc(-c2nc(-c3ccc4c(-c5ccc6ccccc6c5)cccc4c3)nc(-c3cccc4sc5ccccc5c34)n2)cc1 ; CC1(C)c2ccccc2-c2c(-c3nc(-c4ccccc4)nc(-c4ccc5c(-c6ccc7ccccc7c6)cccc5c4)n3)cccc21 ; c1ccc(-c2nc(-c3ccc4c(-c5ccc6ccccc6c5)cccc4c3)nc(-c3ccc4ccc5oc6ccccc6c5c4c3)n2)cc1]

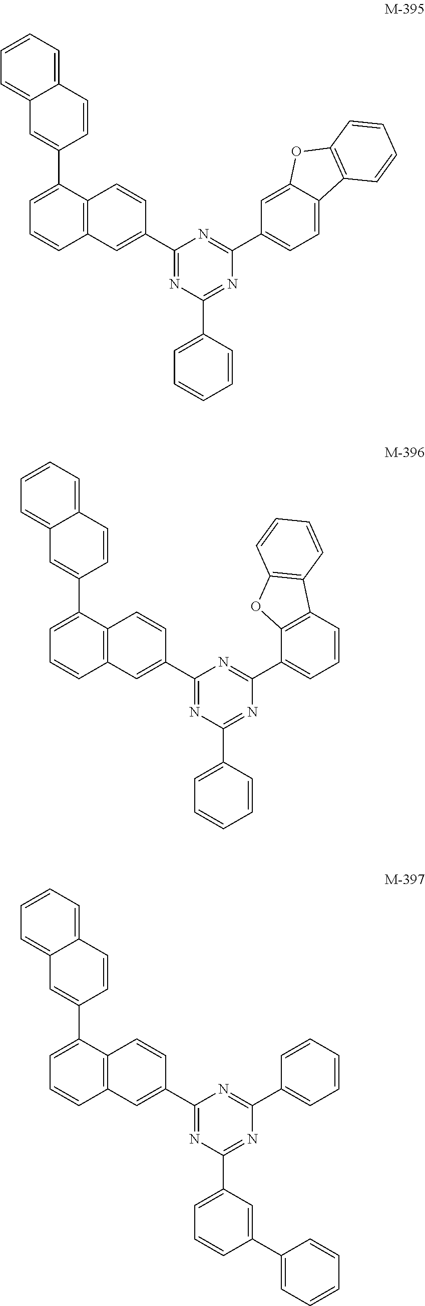 [SMILES: c1ccc(-c2nc(-c3ccc4c(-c5ccc6ccccc6c5)cccc4c3)nc(-c3ccc4c(c3)oc3ccccc34)n2)cc1 ; c1ccc(-c2nc(-c3ccc4c(-c5ccc6ccccc6c5)cccc4c3)nc(-c3cccc4c3oc3ccccc34)n2)cc1 ; c1ccc(-c2cccc(-c3nc(-c4ccccc4)nc(-c4ccc5c(-c6ccc7ccccc7c6)cccc5c4)n3)c2)cc1]

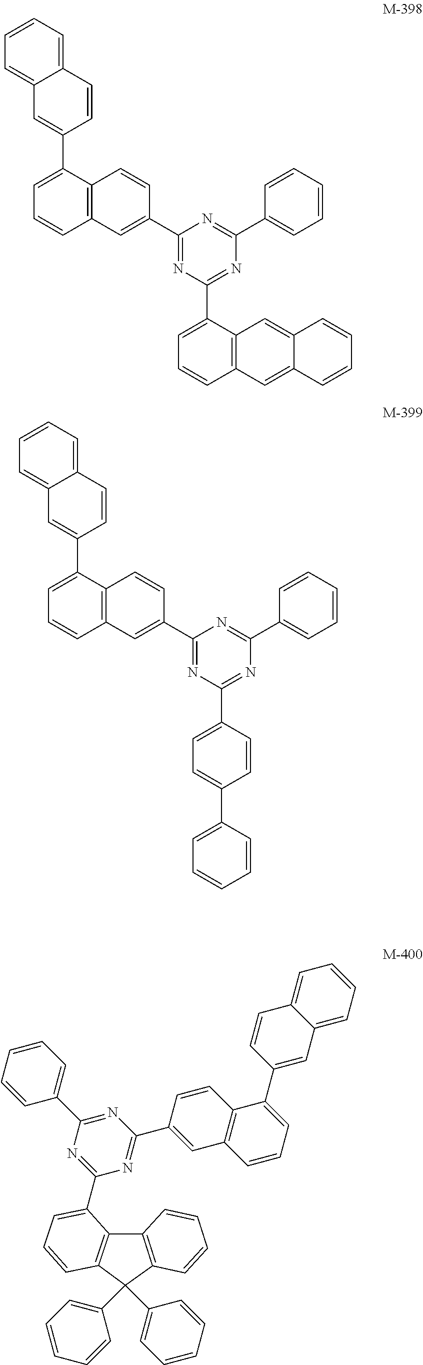 [SMILES: c1ccc(-c2nc(-c3ccc4c(-c5ccc6ccccc6c5)cccc4c3)nc(-c3cccc4cc5ccccc5cc34)n2)cc1 ; c1ccc(-c2ccc(-c3nc(-c4ccccc4)nc(-c4ccc5c(-c6ccc7ccccc7c6)cccc5c4)n3)cc2)cc1 ; c1ccc(-c2nc(-c3ccc4c(-c5ccc6ccccc6c5)cccc4c3)nc(-c3cccc4c3-c3ccccc3C4(c3ccccc3)c3ccccc3)n2)cc1]

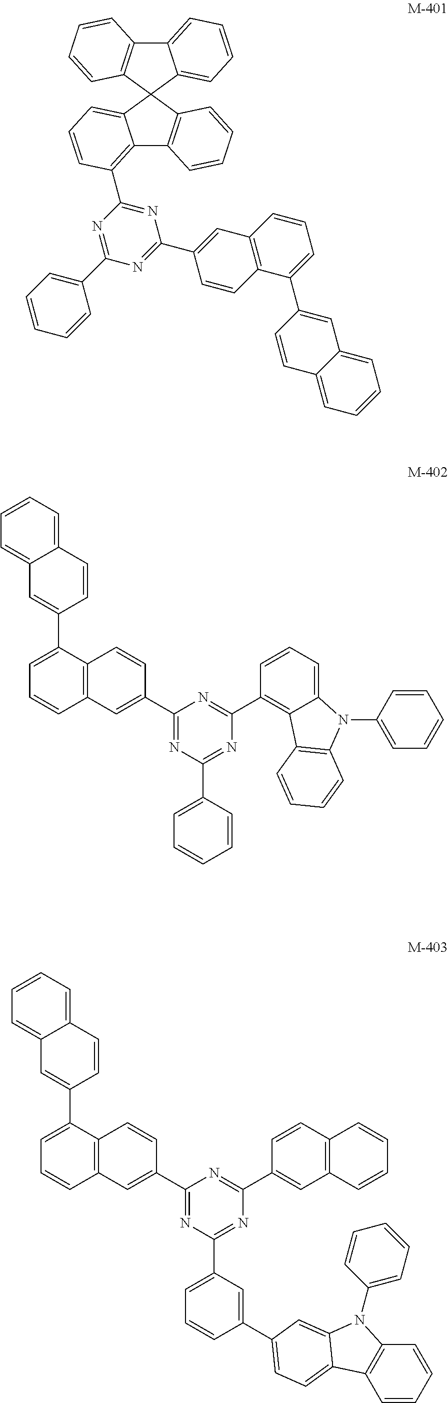 [SMILES: c1ccc(-c2nc(-c3ccc4c(-c5ccc6ccccc6c5)cccc4c3)nc(-c3cccc4c3-c3ccccc3C43c4ccccc4-c4ccccc43)n2)cc1 ; c1ccc(-c2nc(-c3ccc4c(-c5ccc6ccccc6c5)cccc4c3)nc(-c3cccc4c3c3ccccc3n4-c3ccccc3)n2)cc1 ; c1ccc(-n2c3ccccc3c3ccc(-c4cccc(-c5nc(-c6ccc7ccccc7c6)nc(-c6ccc7c(-c8ccc9ccccc9c8)cccc7c6)n5)c4)cc32)cc1]

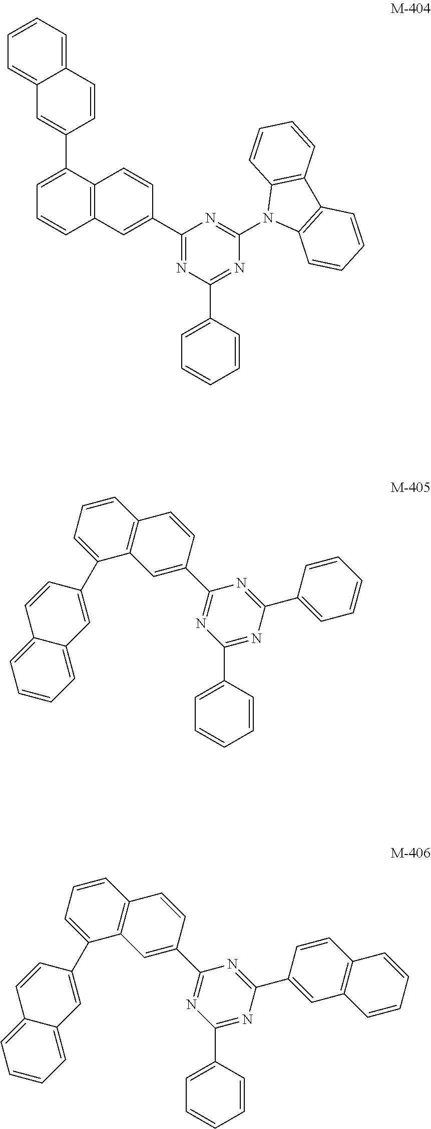 [SMILES: c1ccc(-c2nc(-c3ccc4c(-c5ccc6ccccc6c5)cccc4c3)nc(-n3c4ccccc4c4ccccc43)n2)cc1 ; c1ccc(-c2nc(-c3ccccc3)nc(-c3ccc4cccc(-c5ccc6ccccc6c5)c4c3)n2)cc1 ; c1ccc(-c2nc(-c3ccc4ccccc4c3)nc(-c3ccc4cccc(-c5ccc6ccccc6c5)c4c3)n2)cc1]

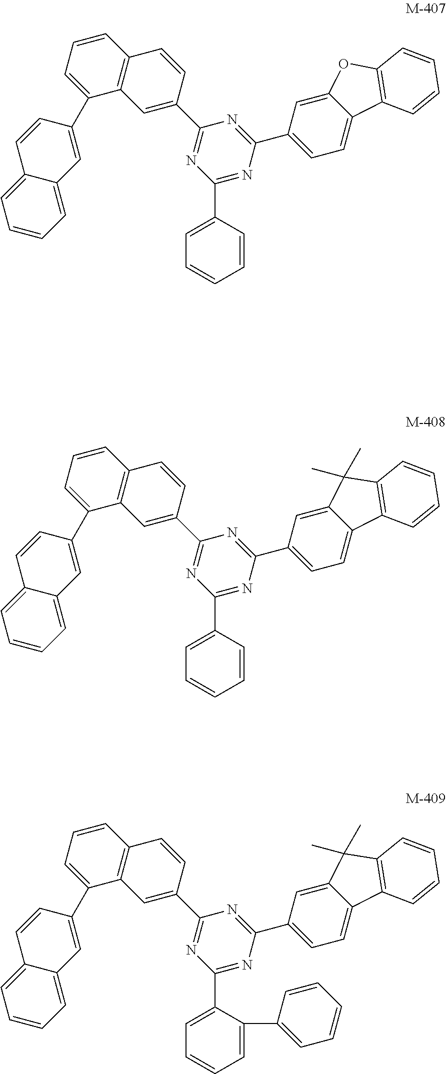 [SMILES: c1ccc(-c2nc(-c3ccc4c(c3)oc3ccccc34)nc(-c3ccc4cccc(-c5ccc6ccccc6c5)c4c3)n2)cc1 ; CC1(C)c2ccccc2-c2ccc(-c3nc(-c4ccccc4)nc(-c4ccc5cccc(-c6ccc7ccccc7c6)c5c4)n3)cc21 ; CC1(C)c2ccccc2-c2ccc(-c3nc(-c4ccc5cccc(-c6ccc7ccccc7c6)c5c4)nc(-c4ccccc4-c4ccccc4)n3)cc21]

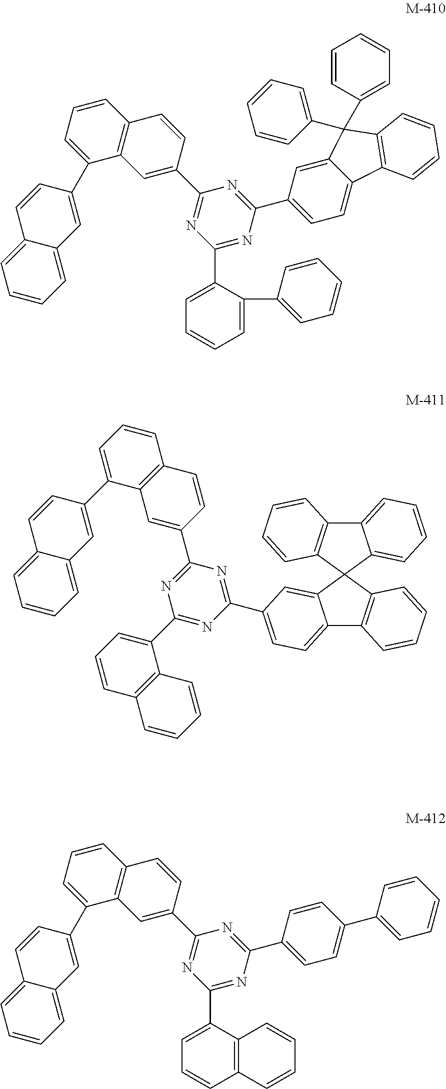 [SMILES: c1ccc(-c2ccccc2-c2nc(-c3ccc4c(c3)C(c3ccccc3)(c3ccccc3)c3ccccc3-4)nc(-c3ccc4cccc(-c5ccc6ccccc6c5)c4c3)n2)cc1 ; c1ccc2c(c1)-c1ccccc1C21c2ccccc2-c2ccc(-c3nc(-c4ccc5cccc(-c6ccc7ccccc7c6)c5c4)nc(-c4cccc5ccccc45)n3)cc21 ; c1ccc(-c2ccc(-c3nc(-c4ccc5cccc(-c6ccc7ccccc7c6)c5c4)nc(-c4cccc5ccccc45)n3)cc2)cc1]

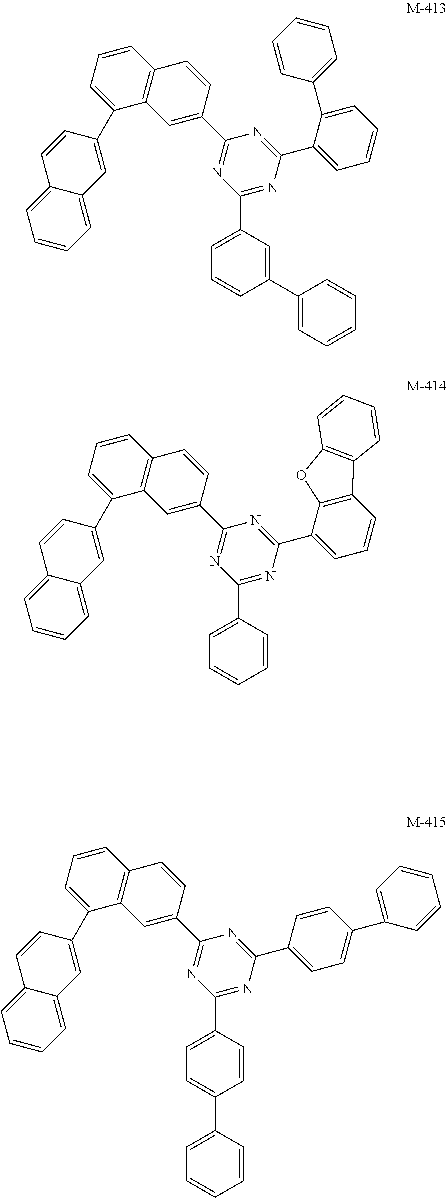 [SMILES: c1ccc(-c2cccc(-c3nc(-c4ccc5cccc(-c6ccc7ccccc7c6)c5c4)nc(-c4ccccc4-c4ccccc4)n3)c2)cc1 ; c1ccc(-c2nc(-c3ccc4cccc(-c5ccc6ccccc6c5)c4c3)nc(-c3cccc4c3oc3ccccc34)n2)cc1 ; c1ccc(-c2ccc(-c3nc(-c4ccc(-c5ccccc5)cc4)nc(-c4ccc5cccc(-c6ccc7ccccc7c6)c5c4)n3)cc2)cc1]

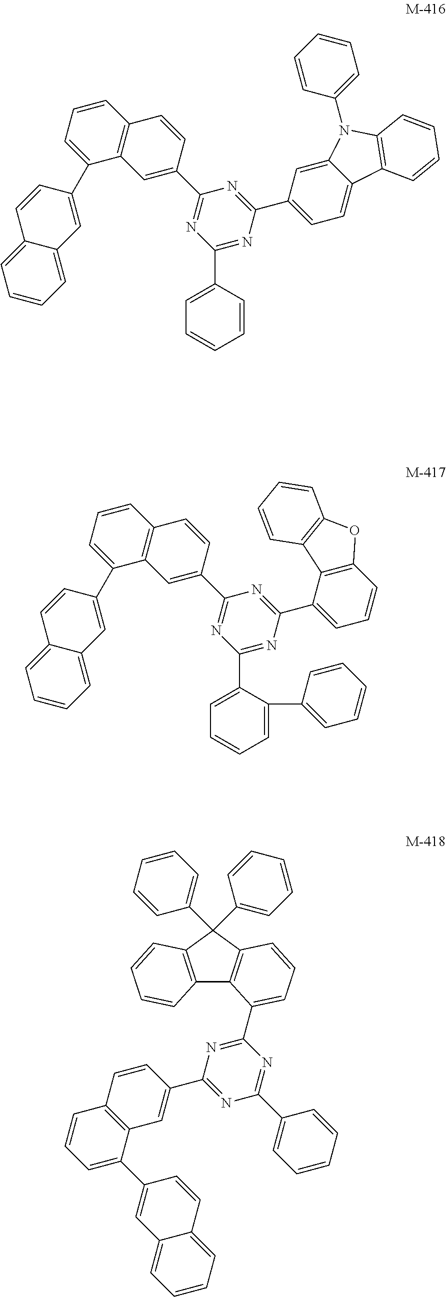 [SMILES: c1ccc(-c2nc(-c3ccc4cccc(-c5ccc6ccccc6c5)c4c3)nc(-c3ccc4c5ccccc5n(-c5ccccc5)c4c3)n2)cc1 ; c1ccc(-c2ccccc2-c2nc(-c3ccc4cccc(-c5ccc6ccccc6c5)c4c3)nc(-c3cccc4oc5ccccc5c34)n2)cc1 ; c1ccc(-c2nc(-c3ccc4cccc(-c5ccc6ccccc6c5)c4c3)nc(-c3cccc4c3-c3ccccc3C4(c3ccccc3)c3ccccc3)n2)cc1]

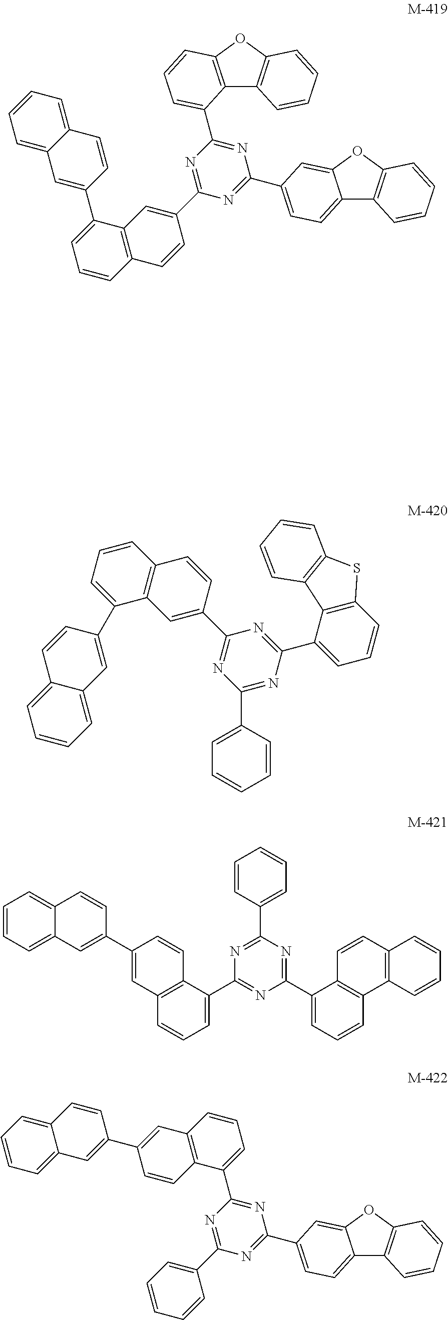 [SMILES: c1ccc2cc(-c3cccc4ccc(-c5nc(-c6ccc7c(c6)oc6ccccc67)nc(-c6cccc7oc8ccccc8c67)n5)cc34)ccc2c1 ; c1ccc(-c2nc(-c3ccc4cccc(-c5ccc6ccccc6c5)c4c3)nc(-c3cccc4sc5ccccc5c34)n2)cc1 ; c1ccc(-c2nc(-c3cccc4cc(-c5ccc6ccccc6c5)ccc34)nc(-c3cccc4c3ccc3ccccc34)n2)cc1 ; c1ccc(-c2nc(-c3ccc4c(c3)oc3ccccc34)nc(-c3cccc4cc(-c5ccc6ccccc6c5)ccc34)n2)cc1]

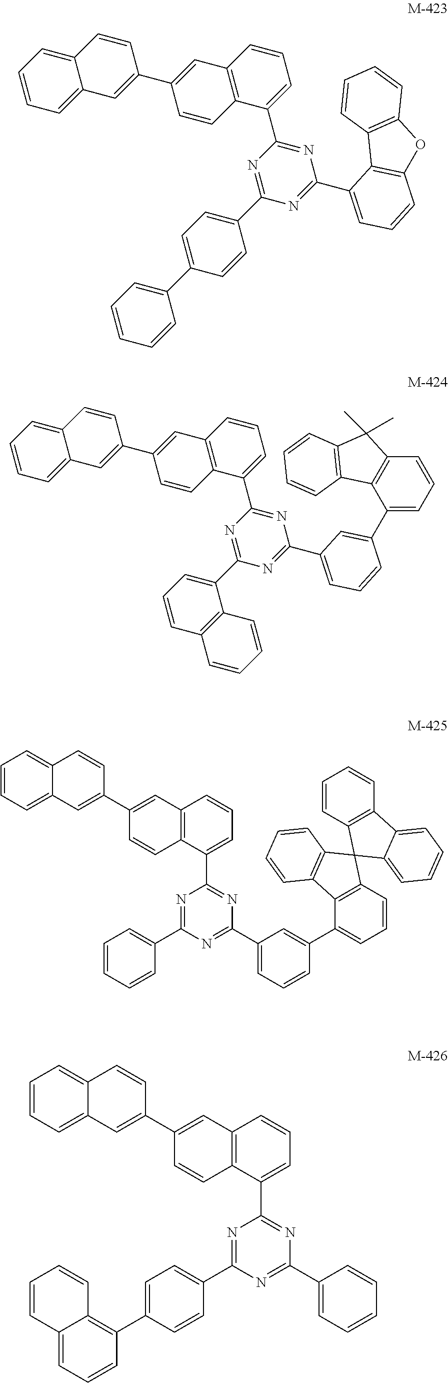 [SMILES: c1ccc(-c2ccc(-c3nc(-c4cccc5cc(-c6ccc7ccccc7c6)ccc45)nc(-c4cccc5oc6ccccc6c45)n3)cc2)cc1 ; CC1(C)c2ccccc2-c2c(-c3cccc(-c4nc(-c5cccc6ccccc56)nc(-c5cccc6cc(-c7ccc8ccccc8c7)ccc56)n4)c3)cccc21 ; c1ccc(-c2nc(-c3cccc(-c4cccc5c4-c4ccccc4C54c5ccccc5-c5ccccc54)c3)nc(-c3cccc4cc(-c5ccc6ccccc6c5)ccc34)n2)cc1 ; c1ccc(-c2nc(-c3ccc(-c4cccc5ccccc45)cc3)nc(-c3cccc4cc(-c5ccc6ccccc6c5)ccc34)n2)cc1]

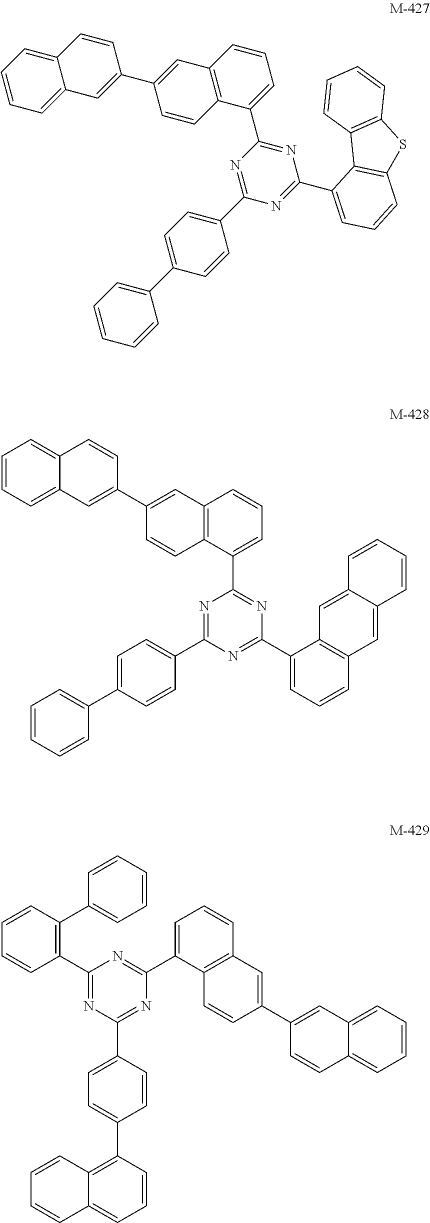 [SMILES: c1ccc(-c2ccc(-c3nc(-c4cccc5cc(-c6ccc7ccccc7c6)ccc45)nc(-c4cccc5sc6ccccc6c45)n3)cc2)cc1 ; c1ccc(-c2ccc(-c3nc(-c4cccc5cc(-c6ccc7ccccc7c6)ccc45)nc(-c4cccc5cc6ccccc6cc45)n3)cc2)cc1 ; c1ccc(-c2ccccc2-c2nc(-c3ccc(-c4cccc5ccccc45)cc3)nc(-c3cccc4cc(-c5ccc6ccccc6c5)ccc34)n2)cc1]

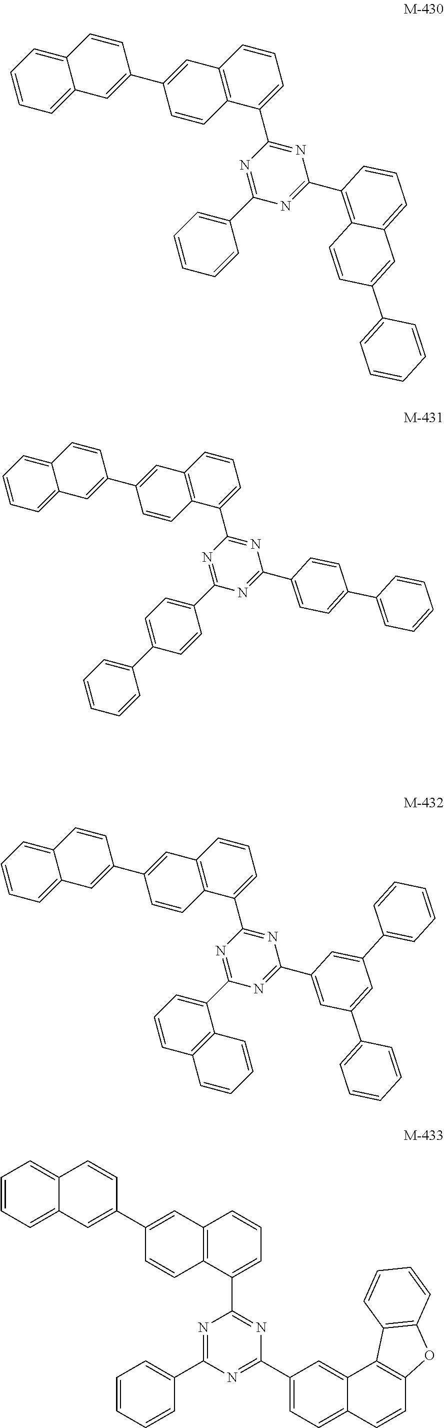 [SMILES: c1ccc(-c2ccc3c(-c4nc(-c5ccccc5)nc(-c5cccc6cc(-c7ccc8ccccc8c7)ccc56)n4)cccc3c2)cc1 ; c1ccc(-c2ccc(-c3nc(-c4ccc(-c5ccccc5)cc4)nc(-c4cccc5cc(-c6ccc7ccccc7c6)ccc45)n3)cc2)cc1 ; c1ccc(-c2cc(-c3ccccc3)cc(-c3nc(-c4cccc5ccccc45)nc(-c4cccc5cc(-c6ccc7ccccc7c6)ccc45)n3)c2)cc1 ; c1ccc(-c2nc(-c3ccc4ccc5oc6ccccc6c5c4c3)nc(-c3cccc4cc(-c5ccc6ccccc6c5)ccc34)n2)cc1]

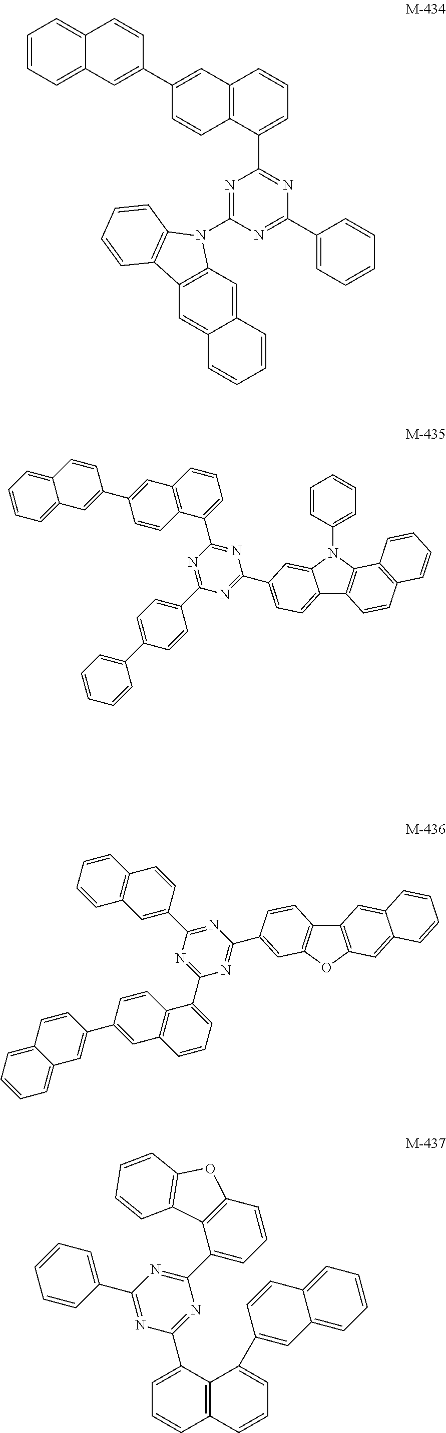 [SMILES: c1ccc(-c2nc(-c3cccc4cc(-c5ccc6ccccc6c5)ccc34)nc(-n3c4ccccc4c4cc5ccccc5cc43)n2)cc1 ; c1ccc(-c2ccc(-c3nc(-c4ccc5c6ccc7ccccc7c6n(-c6ccccc6)c5c4)nc(-c4cccc5cc(-c6ccc7ccccc7c6)ccc45)n3)cc2)cc1 ; c1ccc2cc(-c3ccc4c(-c5nc(-c6ccc7ccccc7c6)nc(-c6ccc7c(c6)oc6cc8ccccc8cc67)n5)cccc4c3)ccc2c1 ; c1ccc(-c2nc(-c3cccc4cccc(-c5ccc6ccccc6c5)c34)nc(-c3cccc4oc5ccccc5c34)n2)cc1]

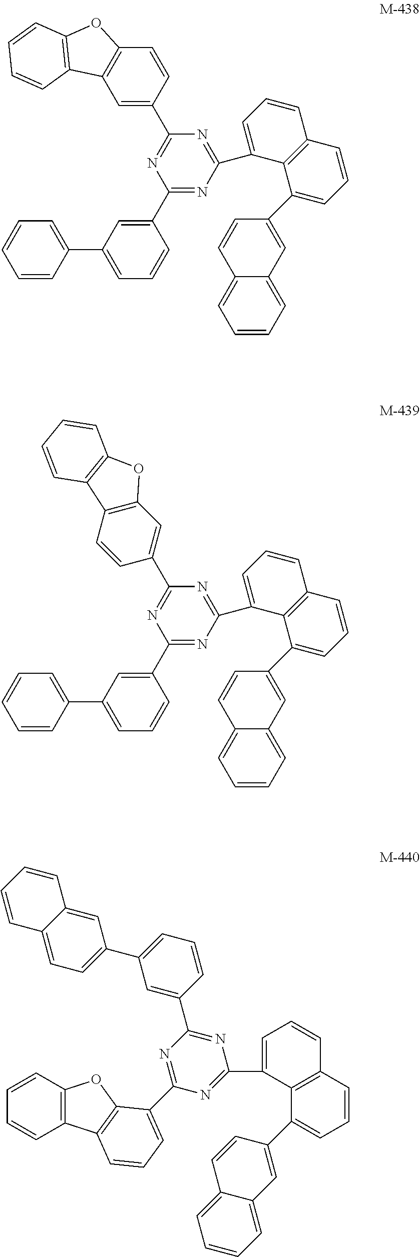 [SMILES: c1ccc(-c2cccc(-c3nc(-c4ccc5oc6ccccc6c5c4)nc(-c4cccc5cccc(-c6ccc7ccccc7c6)c45)n3)c2)cc1 ; c1ccc(-c2cccc(-c3nc(-c4ccc5c(c4)oc4ccccc45)nc(-c4cccc5cccc(-c6ccc7ccccc7c6)c45)n3)c2)cc1 ; c1cc(-c2ccc3ccccc3c2)cc(-c2nc(-c3cccc4c3oc3ccccc34)nc(-c3cccc4cccc(-c5ccc6ccccc6c5)c34)n2)c1]

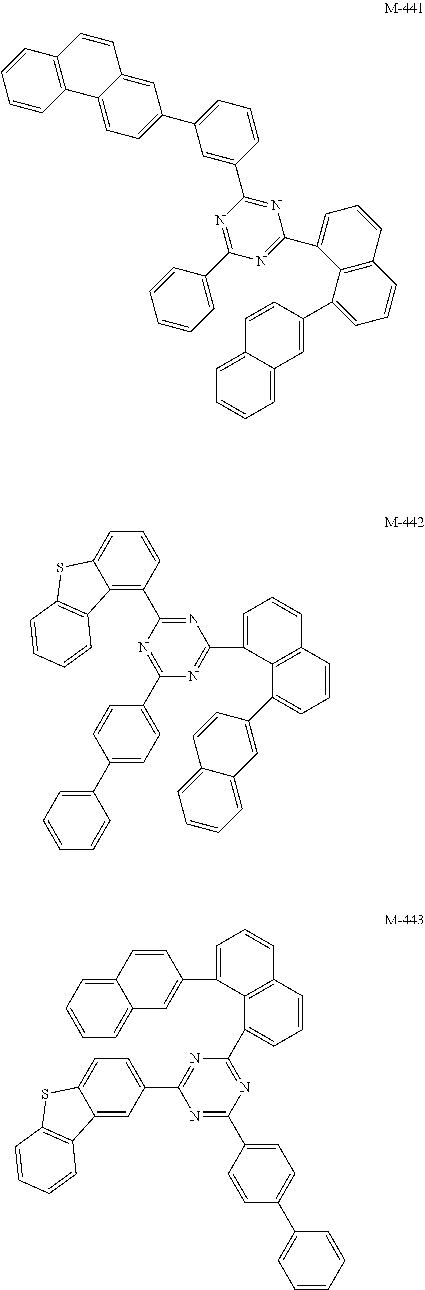 [SMILES: c1ccc(-c2nc(-c3cccc(-c4ccc5c(ccc6ccccc65)c4)c3)nc(-c3cccc4cccc(-c5ccc6ccccc6c5)c34)n2)cc1 ; c1ccc(-c2ccc(-c3nc(-c4cccc5cccc(-c6ccc7ccccc7c6)c45)nc(-c4cccc5sc6ccccc6c45)n3)cc2)cc1 ; c1ccc(-c2ccc(-c3nc(-c4ccc5sc6ccccc6c5c4)nc(-c4cccc5cccc(-c6ccc7ccccc7c6)c45)n3)cc2)cc1]

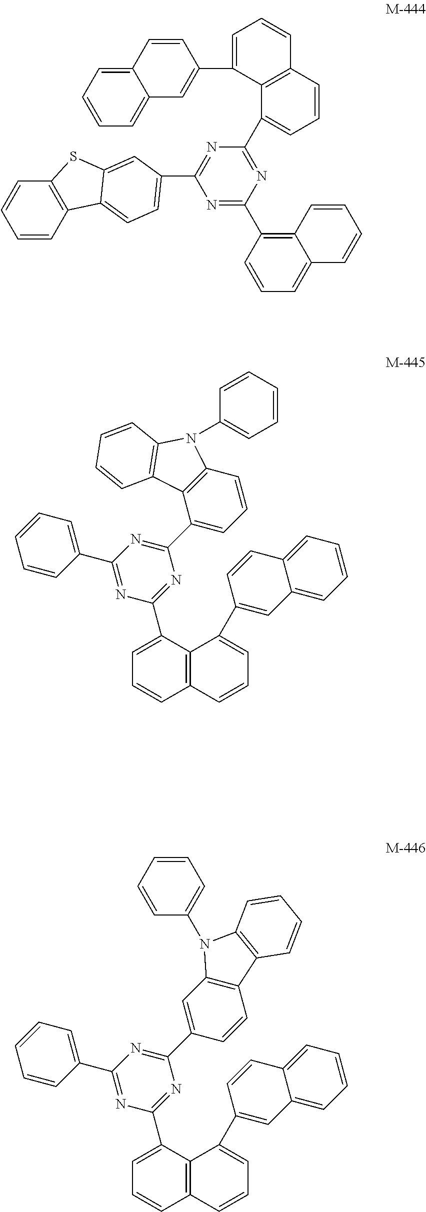 [SMILES: c1ccc2cc(-c3cccc4cccc(-c5nc(-c6ccc7c(c6)sc6ccccc67)nc(-c6cccc7ccccc67)n5)c34)ccc2c1 ; c1ccc(-c2nc(-c3cccc4cccc(-c5ccc6ccccc6c5)c34)nc(-c3cccc4c3c3ccccc3n4-c3ccccc3)n2)cc1 ; c1ccc(-c2nc(-c3ccc4c5ccccc5n(-c5ccccc5)c4c3)nc(-c3cccc4cccc(-c5ccc6ccccc6c5)c34)n2)cc1]

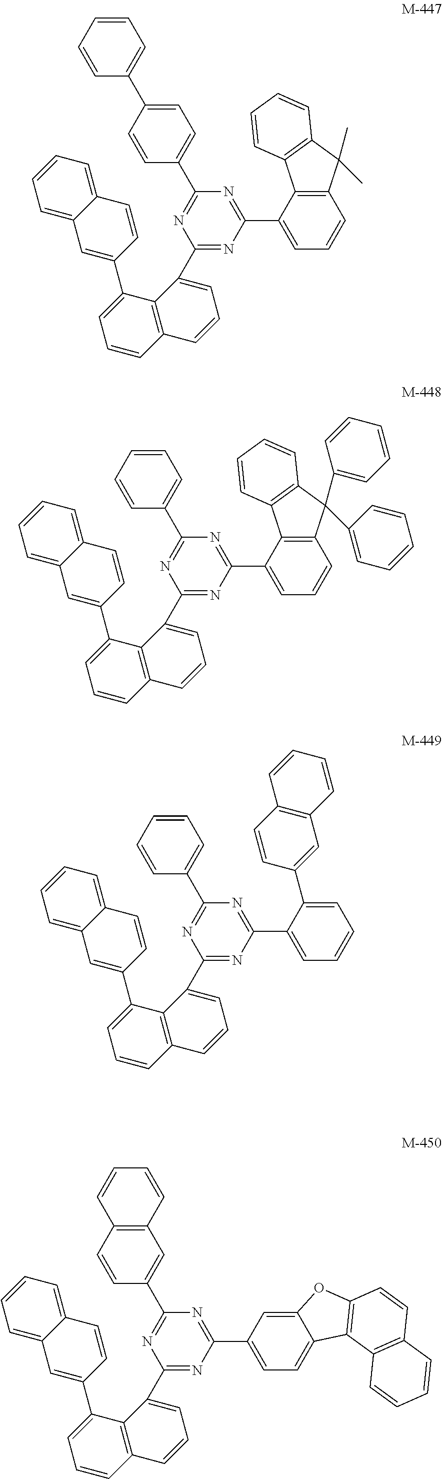 [SMILES: CC1(C)c2ccccc2-c2c(-c3nc(-c4ccc(-c5ccccc5)cc4)nc(-c4cccc5cccc(-c6ccc7ccccc7c6)c45)n3)cccc21 ; c1ccc(-c2nc(-c3cccc4c3-c3ccccc3C4(c3ccccc3)c3ccccc3)nc(-c3cccc4cccc(-c5ccc6ccccc6c5)c34)n2)cc1 ; c1ccc(-c2nc(-c3ccccc3-c3ccc4ccccc4c3)nc(-c3cccc4cccc(-c5ccc6ccccc6c5)c34)n2)cc1 ; c1ccc2cc(-c3nc(-c4ccc5c(c4)oc4ccc6ccccc6c45)nc(-c4cccc5cccc(-c6ccc7ccccc7c6)c45)n3)ccc2c1]

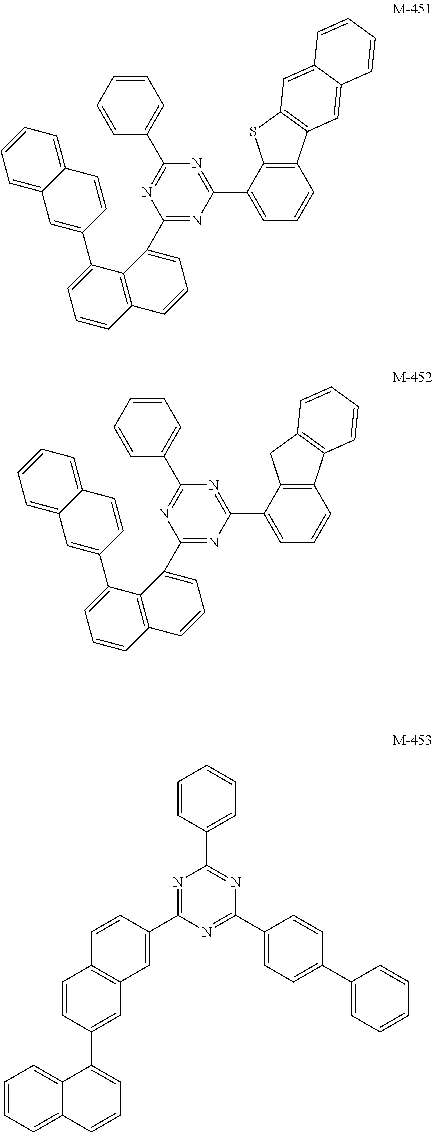 [SMILES: c1ccc(-c2nc(-c3cccc4c3sc3cc5ccccc5cc34)nc(-c3cccc4cccc(-c5ccc6ccccc6c5)c34)n2)cc1 ; c1ccc(-c2nc(-c3cccc4c3Cc3ccccc3-4)nc(-c3cccc4cccc(-c5ccc6ccccc6c5)c34)n2)cc1 ; c1ccc(-c2ccc(-c3nc(-c4ccccc4)nc(-c4ccc5ccc(-c6cccc7ccccc67)cc5c4)n3)cc2)cc1]

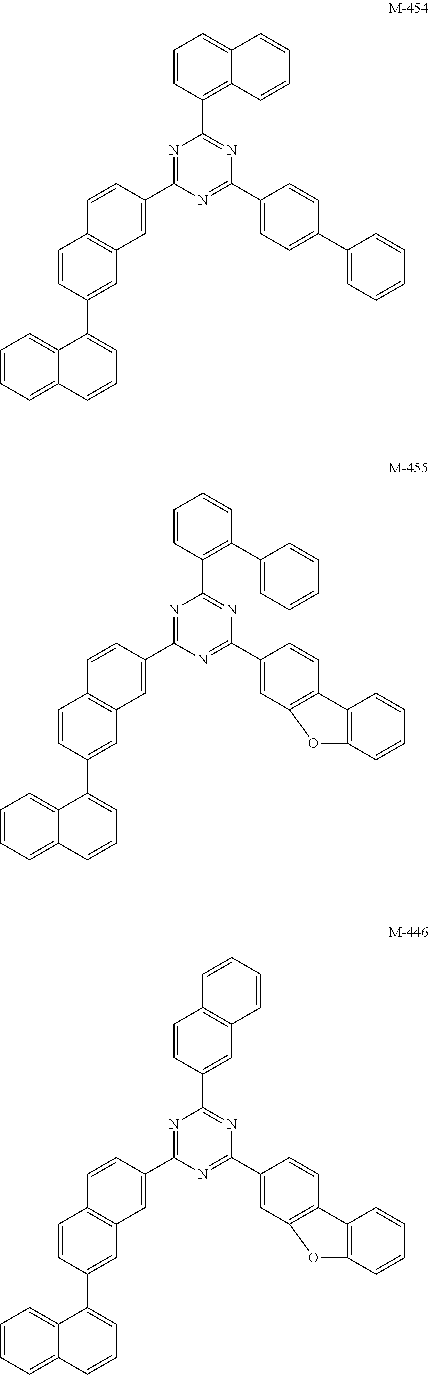 [SMILES: c1ccc(-c2ccc(-c3nc(-c4ccc5ccc(-c6cccc7ccccc67)cc5c4)nc(-c4cccc5ccccc45)n3)cc2)cc1 ; c1ccc(-c2ccccc2-c2nc(-c3ccc4ccc(-c5cccc6ccccc56)cc4c3)nc(-c3ccc4c(c3)oc3ccccc34)n2)cc1 ; c1ccc2cc(-c3nc(-c4ccc5ccc(-c6cccc7ccccc67)cc5c4)nc(-c4ccc5c(c4)oc4ccccc45)n3)ccc2c1]

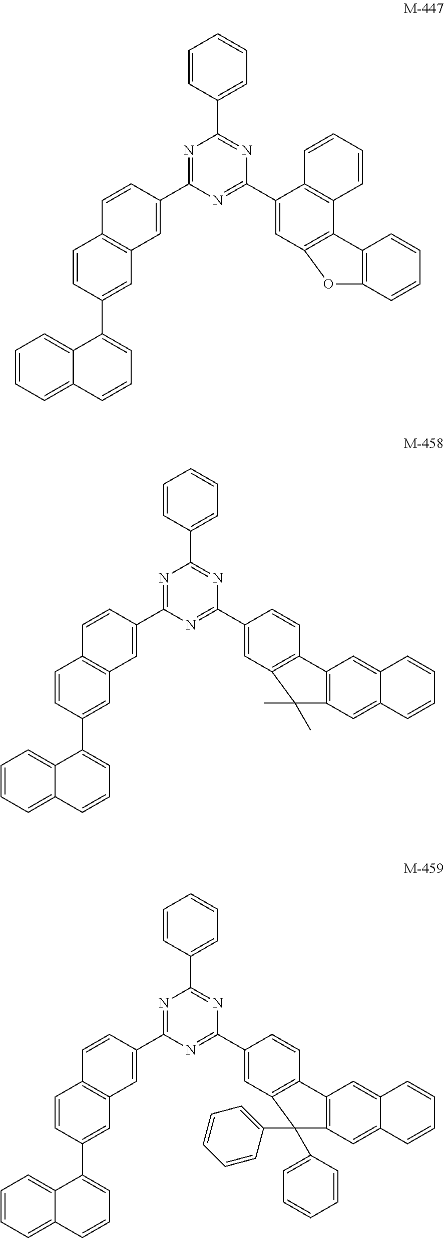 [SMILES: c1ccc(-c2nc(-c3ccc4ccc(-c5cccc6ccccc56)cc4c3)nc(-c3cc4oc5ccccc5c4c4ccccc34)n2)cc1 ; CC1(C)c2cc(-c3nc(-c4ccccc4)nc(-c4ccc5ccc(-c6cccc7ccccc67)cc5c4)n3)ccc2-c2cc3ccccc3cc21 ; c1ccc(-c2nc(-c3ccc4c(c3)C(c3ccccc3)(c3ccccc3)c3cc5ccccc5cc3-4)nc(-c3ccc4ccc(-c5cccc6ccccc56)cc4c3)n2)cc1]

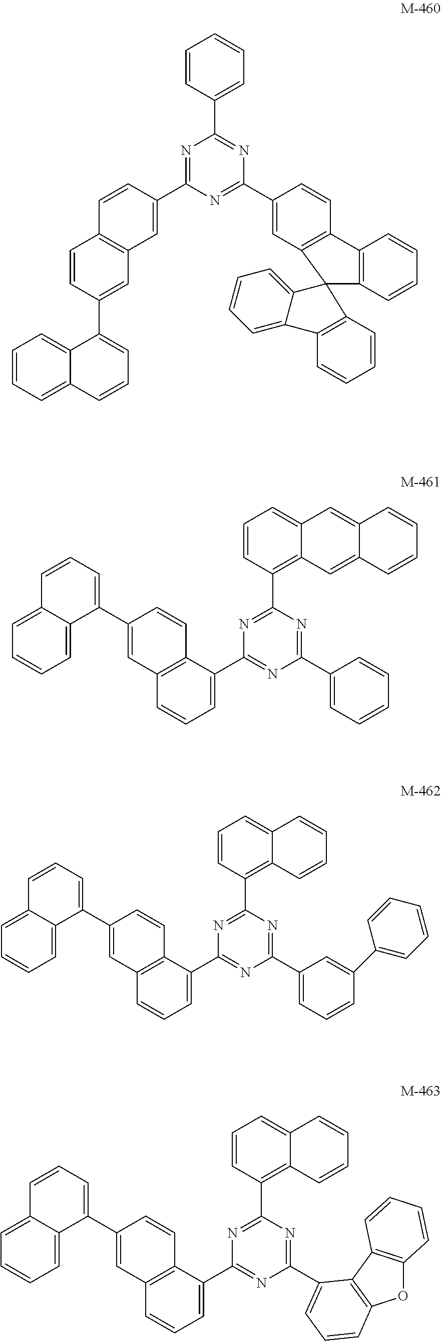 [SMILES: c1ccc(-c2nc(-c3ccc4c(c3)C3(c5ccccc5-c5ccccc53)c3ccccc3-4)nc(-c3ccc4ccc(-c5cccc6ccccc56)cc4c3)n2)cc1 ; c1ccc(-c2nc(-c3cccc4cc(-c5cccc6ccccc56)ccc34)nc(-c3cccc4cc5ccccc5cc34)n2)cc1 ; c1ccc(-c2cccc(-c3nc(-c4cccc5ccccc45)nc(-c4cccc5cc(-c6cccc7ccccc67)ccc45)n3)c2)cc1 ; c1ccc2c(-c3ccc4c(-c5nc(-c6cccc7ccccc67)nc(-c6cccc7oc8ccccc8c67)n5)cccc4c3)cccc2c1]

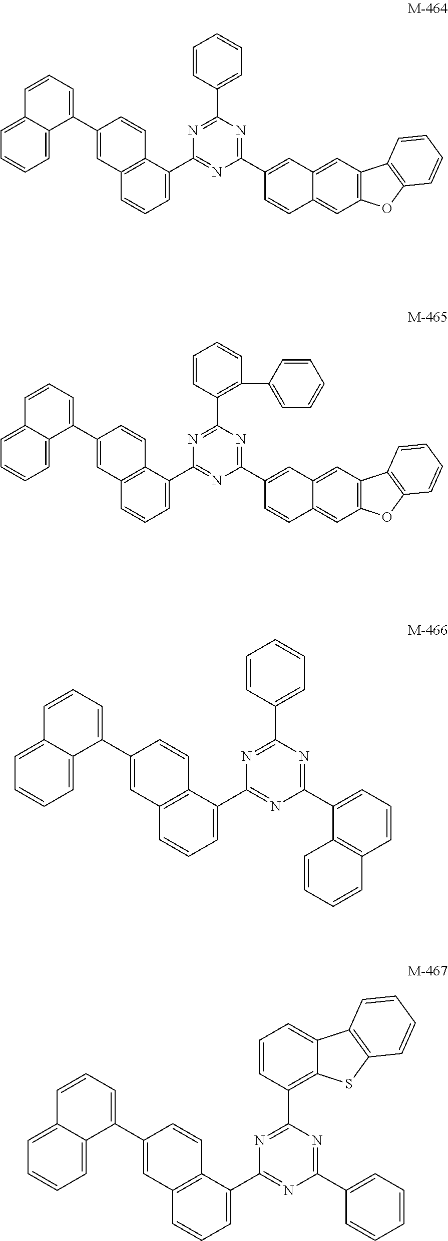 [SMILES: c1ccc(-c2nc(-c3ccc4cc5oc6ccccc6c5cc4c3)nc(-c3cccc4cc(-c5cccc6ccccc56)ccc34)n2)cc1 ; c1ccc(-c2ccccc2-c2nc(-c3ccc4cc5oc6ccccc6c5cc4c3)nc(-c3cccc4cc(-c5cccc6ccccc56)ccc34)n2)cc1 ; c1ccc(-c2nc(-c3cccc4ccccc34)nc(-c3cccc4cc(-c5cccc6ccccc56)ccc34)n2)cc1 ; c1ccc(-c2nc(-c3cccc4cc(-c5cccc6ccccc56)ccc34)nc(-c3cccc4c3sc3ccccc34)n2)cc1]

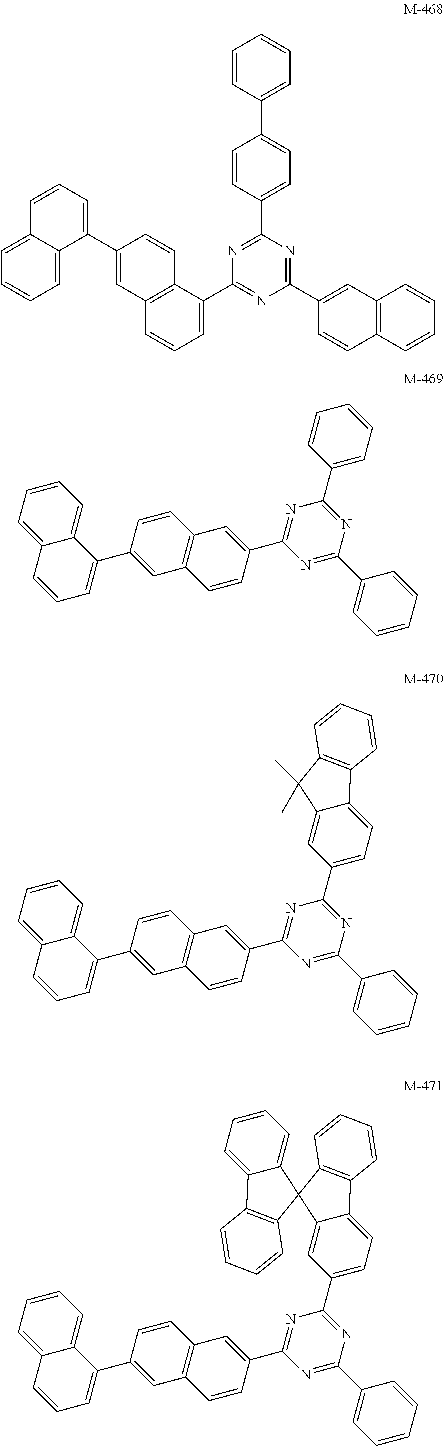 [SMILES: c1ccc(-c2ccc(-c3nc(-c4ccc5ccccc5c4)nc(-c4cccc5cc(-c6cccc7ccccc67)ccc45)n3)cc2)cc1 ; c1ccc(-c2nc(-c3ccccc3)nc(-c3ccc4cc(-c5cccc6ccccc56)ccc4c3)n2)cc1 ; CC1(C)c2ccccc2-c2ccc(-c3nc(-c4ccccc4)nc(-c4ccc5cc(-c6cccc7ccccc67)ccc5c4)n3)cc21 ; c1ccc(-c2nc(-c3ccc4c(c3)C3(c5ccccc5-c5ccccc53)c3ccccc3-4)nc(-c3ccc4cc(-c5cccc6ccccc56)ccc4c3)n2)cc1]

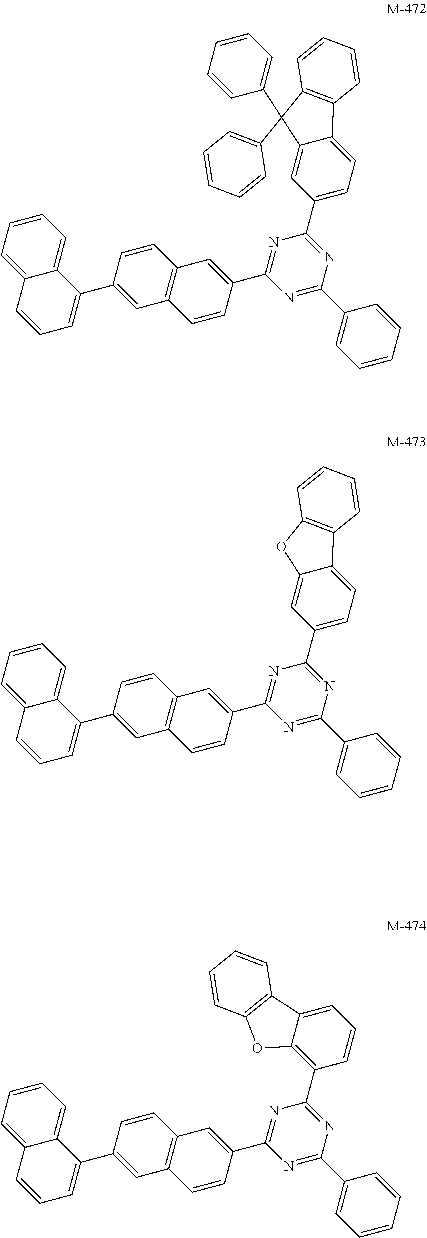 [SMILES: c1ccc(-c2nc(-c3ccc4c(c3)C(c3ccccc3)(c3ccccc3)c3ccccc3-4)nc(-c3ccc4cc(-c5cccc6ccccc56)ccc4c3)n2)cc1 ; c1ccc(-c2nc(-c3ccc4cc(-c5cccc6ccccc56)ccc4c3)nc(-c3ccc4c(c3)oc3ccccc34)n2)cc1 ; c1ccc(-c2nc(-c3ccc4cc(-c5cccc6ccccc56)ccc4c3)nc(-c3cccc4c3oc3ccccc34)n2)cc1]

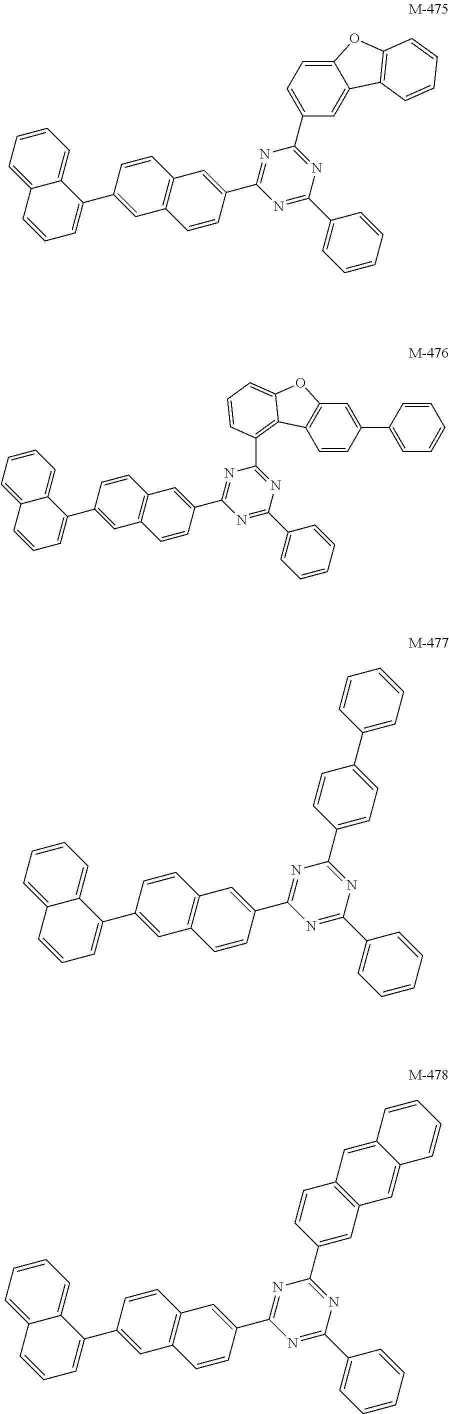 [SMILES: c1ccc(-c2nc(-c3ccc4cc(-c5cccc6ccccc56)ccc4c3)nc(-c3ccc4oc5ccccc5c4c3)n2)cc1 ; c1ccc(-c2ccc3c(c2)oc2cccc(-c4nc(-c5ccccc5)nc(-c5ccc6cc(-c7cccc8ccccc78)ccc6c5)n4)c23)cc1 ; c1ccc(-c2ccc(-c3nc(-c4ccccc4)nc(-c4ccc5cc(-c6cccc7ccccc67)ccc5c4)n3)cc2)cc1 ; c1ccc(-c2nc(-c3ccc4cc(-c5cccc6ccccc56)ccc4c3)nc(-c3ccc4cc5ccccc5cc4c3)n2)cc1]

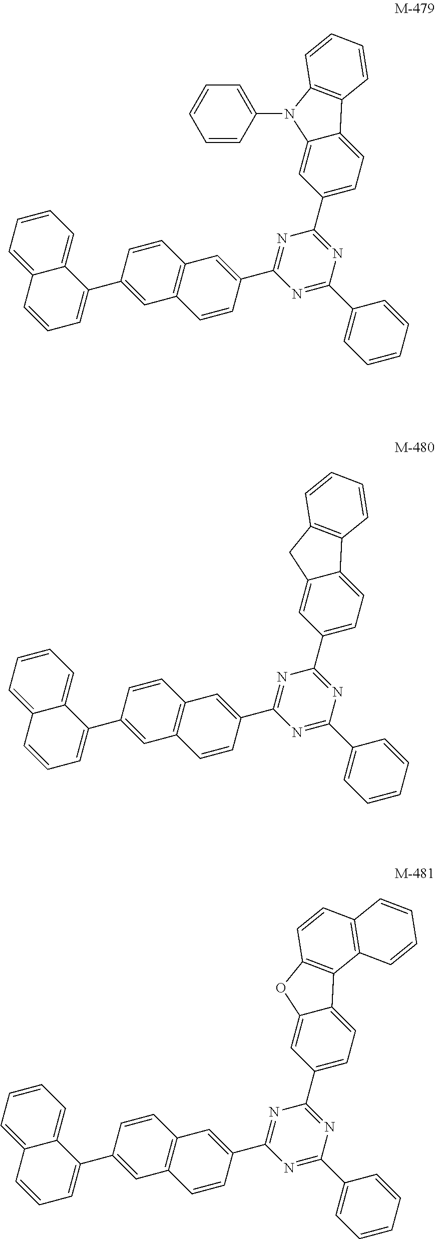 [SMILES: c1ccc(-c2nc(-c3ccc4cc(-c5cccc6ccccc56)ccc4c3)nc(-c3ccc4c5ccccc5n(-c5ccccc5)c4c3)n2)cc1 ; c1ccc(-c2nc(-c3ccc4c(c3)Cc3ccccc3-4)nc(-c3ccc4cc(-c5cccc6ccccc56)ccc4c3)n2)cc1 ; c1ccc(-c2nc(-c3ccc4cc(-c5cccc6ccccc56)ccc4c3)nc(-c3ccc4c(c3)oc3ccc5ccccc5c34)n2)cc1]

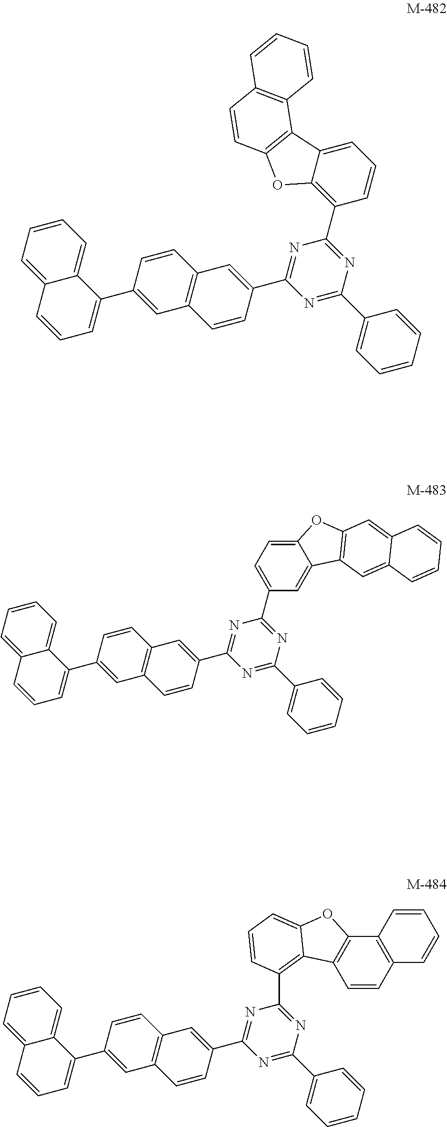 [SMILES: c1ccc(-c2nc(-c3ccc4cc(-c5cccc6ccccc56)ccc4c3)nc(-c3cccc4c3oc3ccc5ccccc5c34)n2)cc1 ; c1ccc(-c2nc(-c3ccc4cc(-c5cccc6ccccc56)ccc4c3)nc(-c3ccc4oc5cc6ccccc6cc5c4c3)n2)cc1 ; c1ccc(-c2nc(-c3ccc4cc(-c5cccc6ccccc56)ccc4c3)nc(-c3cccc4oc5c6ccccc6ccc5c34)n2)cc1]

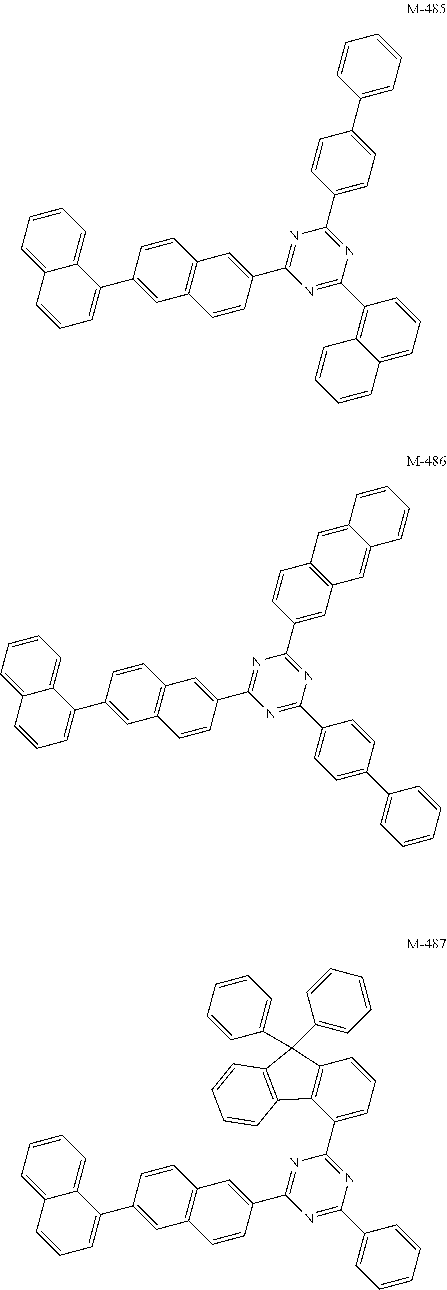 [SMILES: c1ccc(-c2ccc(-c3nc(-c4ccc5cc(-c6cccc7ccccc67)ccc5c4)nc(-c4cccc5ccccc45)n3)cc2)cc1 ; c1ccc(-c2ccc(-c3nc(-c4ccc5cc(-c6cccc7ccccc67)ccc5c4)nc(-c4ccc5cc6ccccc6cc5c4)n3)cc2)cc1 ; c1ccc(-c2nc(-c3ccc4cc(-c5cccc6ccccc56)ccc4c3)nc(-c3cccc4c3-c3ccccc3C4(c3ccccc3)c3ccccc3)n2)cc1]

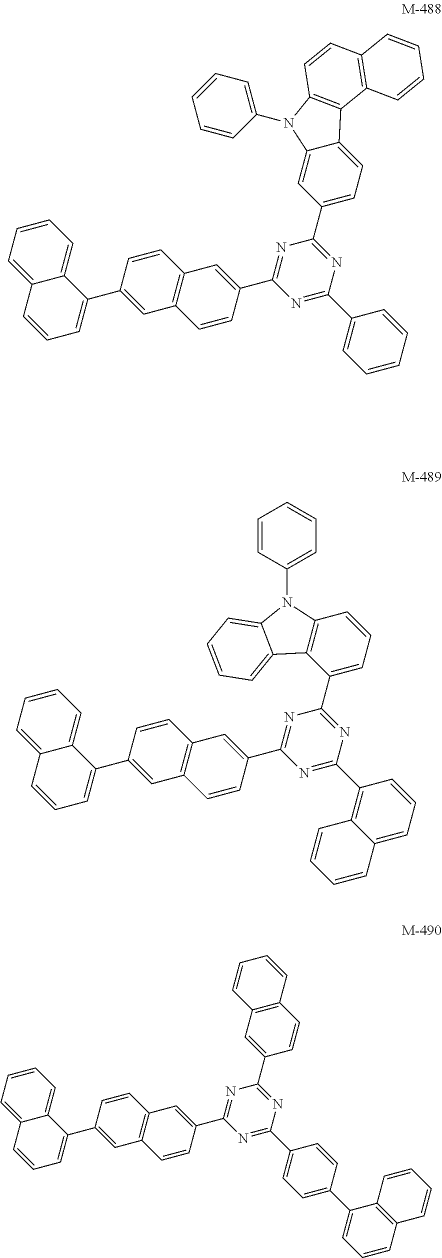 [SMILES: c1ccc(-c2nc(-c3ccc4cc(-c5cccc6ccccc56)ccc4c3)nc(-c3ccc4c5c6ccccc6ccc5n(-c5ccccc5)c4c3)n2)cc1 ; c1ccc(-n2c3ccccc3c3c(-c4nc(-c5ccc6cc(-c7cccc8ccccc78)ccc6c5)nc(-c5cccc6ccccc56)n4)cccc32)cc1 ; c1ccc2cc(-c3nc(-c4ccc(-c5cccc6ccccc56)cc4)nc(-c4ccc5cc(-c6cccc7ccccc67)ccc5c4)n3)ccc2c1]

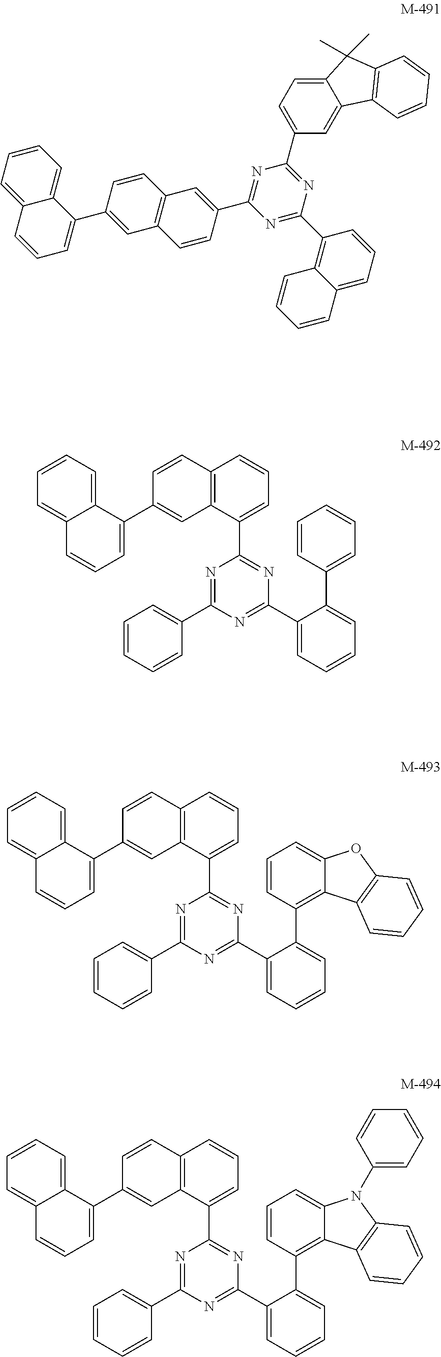 [SMILES: CC1(C)c2ccccc2-c2cc(-c3nc(-c4ccc5cc(-c6cccc7ccccc67)ccc5c4)nc(-c4cccc5ccccc45)n3)ccc21 ; c1ccc(-c2nc(-c3ccccc3-c3ccccc3)nc(-c3cccc4ccc(-c5cccc6ccccc56)cc34)n2)cc1 ; c1ccc(-c2nc(-c3ccccc3-c3cccc4oc5ccccc5c34)nc(-c3cccc4ccc(-c5cccc6ccccc56)cc34)n2)cc1 ; c1ccc(-c2nc(-c3ccccc3-c3cccc4c3c3ccccc3n4-c3ccccc3)nc(-c3cccc4ccc(-c5cccc6ccccc56)cc34)n2)cc1]

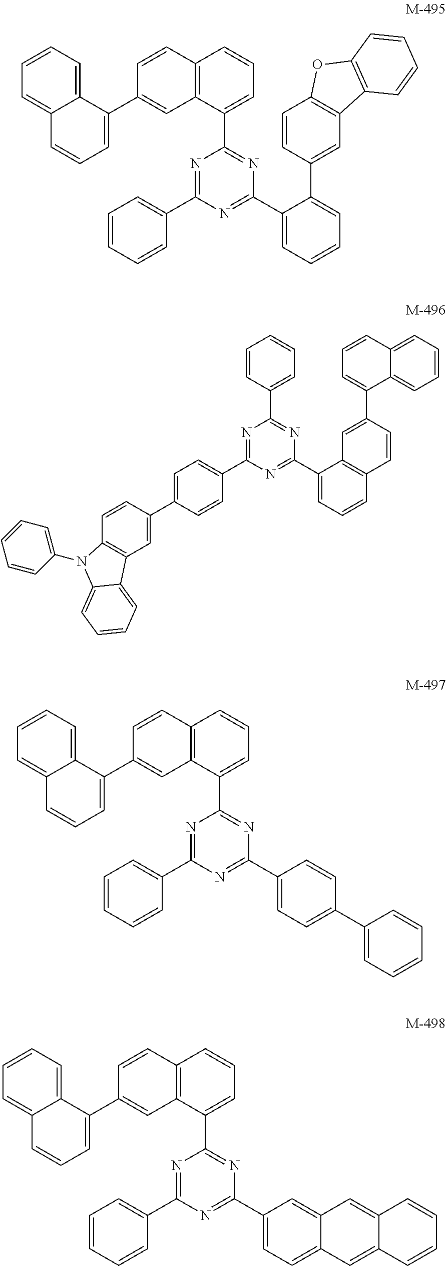 [SMILES: c1ccc(-c2nc(-c3ccccc3-c3ccc4oc5ccccc5c4c3)nc(-c3cccc4ccc(-c5cccc6ccccc56)cc34)n2)cc1 ; c1ccc(-c2nc(-c3ccc(-c4ccc5c(c4)c4ccccc4n5-c4ccccc4)cc3)nc(-c3cccc4ccc(-c5cccc6ccccc56)cc34)n2)cc1 ; c1ccc(-c2ccc(-c3nc(-c4ccccc4)nc(-c4cccc5ccc(-c6cccc7ccccc67)cc45)n3)cc2)cc1 ; c1ccc(-c2nc(-c3ccc4cc5ccccc5cc4c3)nc(-c3cccc4ccc(-c5cccc6ccccc56)cc34)n2)cc1]

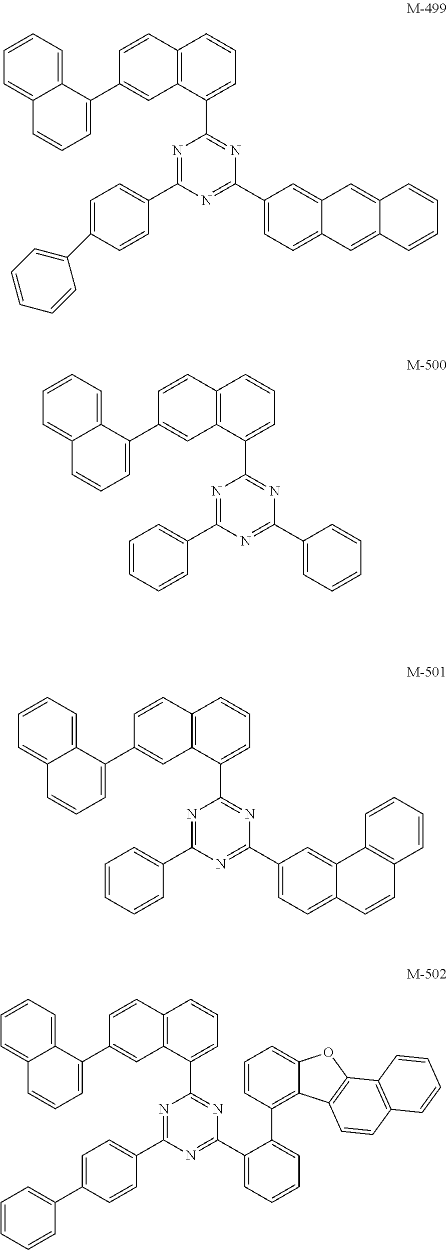 [SMILES: c1ccc(-c2ccc(-c3nc(-c4ccc5cc6ccccc6cc5c4)nc(-c4cccc5ccc(-c6cccc7ccccc67)cc45)n3)cc2)cc1 ; c1ccc(-c2nc(-c3ccccc3)nc(-c3cccc4ccc(-c5cccc6ccccc56)cc34)n2)cc1 ; c1ccc(-c2nc(-c3ccc4ccc5ccccc5c4c3)nc(-c3cccc4ccc(-c5cccc6ccccc56)cc34)n2)cc1 ; c1ccc(-c2ccc(-c3nc(-c4ccccc4-c4cccc5oc6c7ccccc7ccc6c45)nc(-c4cccc5ccc(-c6cccc7ccccc67)cc45)n3)cc2)cc1]

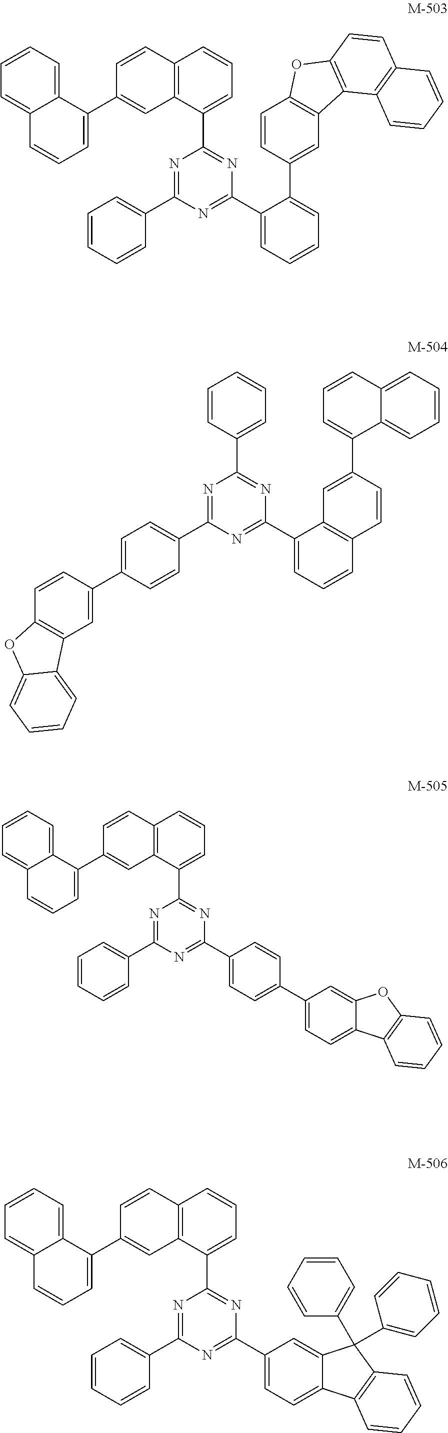 [SMILES: c1ccc(-c2nc(-c3ccccc3-c3ccc4oc5ccc6ccccc6c5c4c3)nc(-c3cccc4ccc(-c5cccc6ccccc56)cc34)n2)cc1 ; c1ccc(-c2nc(-c3ccc(-c4ccc5oc6ccccc6c5c4)cc3)nc(-c3cccc4ccc(-c5cccc6ccccc56)cc34)n2)cc1 ; c1ccc(-c2nc(-c3ccc(-c4ccc5c(c4)oc4ccccc45)cc3)nc(-c3cccc4ccc(-c5cccc6ccccc56)cc34)n2)cc1 ; c1ccc(-c2nc(-c3ccc4c(c3)C(c3ccccc3)(c3ccccc3)c3ccccc3-4)nc(-c3cccc4ccc(-c5cccc6ccccc56)cc34)n2)cc1]

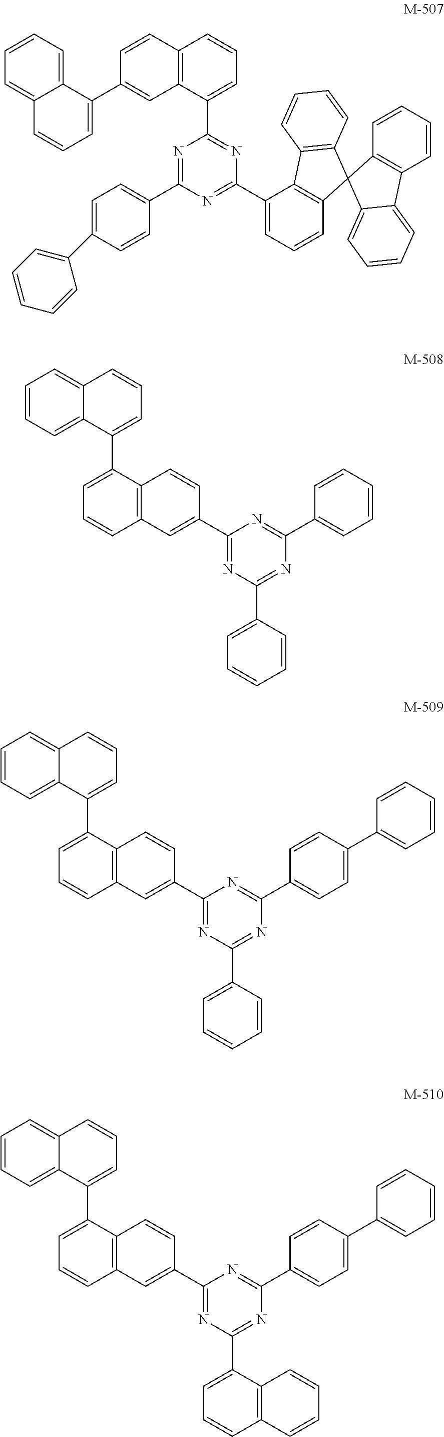 [SMILES: c1ccc(-c2ccc(-c3nc(-c4cccc5c4-c4ccccc4C54c5ccccc5-c5ccccc54)nc(-c4cccc5ccc(-c6cccc7ccccc67)cc45)n3)cc2)cc1 ; c1ccc(-c2nc(-c3ccccc3)nc(-c3ccc4c(-c5cccc6ccccc56)cccc4c3)n2)cc1 ; c1ccc(-c2ccc(-c3nc(-c4ccccc4)nc(-c4ccc5c(-c6cccc7ccccc67)cccc5c4)n3)cc2)cc1 ; c1ccc(-c2ccc(-c3nc(-c4ccc5c(-c6cccc7ccccc67)cccc5c4)nc(-c4cccc5ccccc45)n3)cc2)cc1]

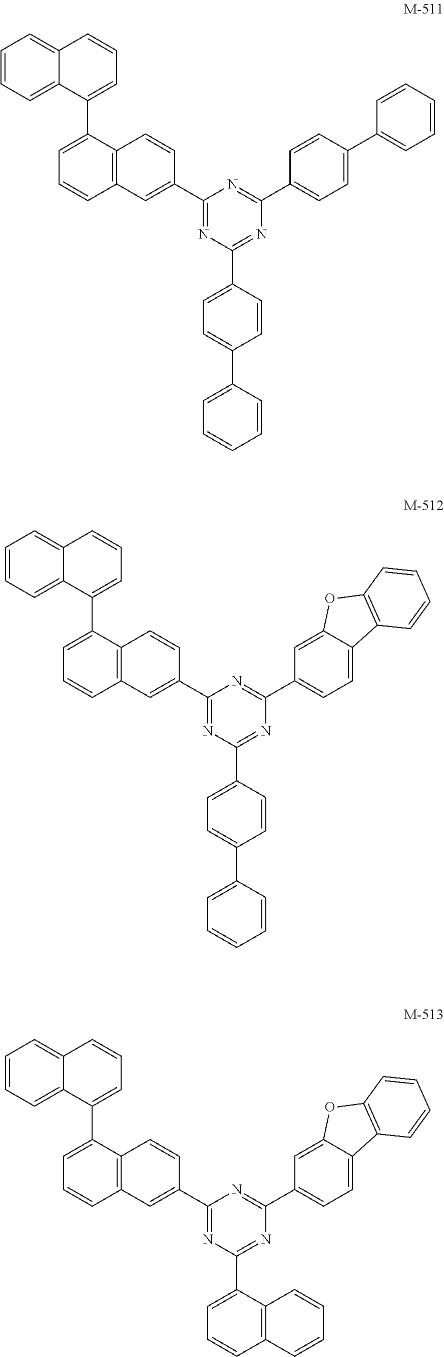 [SMILES: c1ccc(-c2ccc(-c3nc(-c4ccc(-c5ccccc5)cc4)nc(-c4ccc5c(-c6cccc7ccccc67)cccc5c4)n3)cc2)cc1 ; c1ccc(-c2ccc(-c3nc(-c4ccc5c(-c6cccc7ccccc67)cccc5c4)nc(-c4ccc5c(c4)oc4ccccc45)n3)cc2)cc1 ; c1ccc2c(-c3nc(-c4ccc5c(-c6cccc7ccccc67)cccc5c4)nc(-c4ccc5c(c4)oc4ccccc45)n3)cccc2c1]

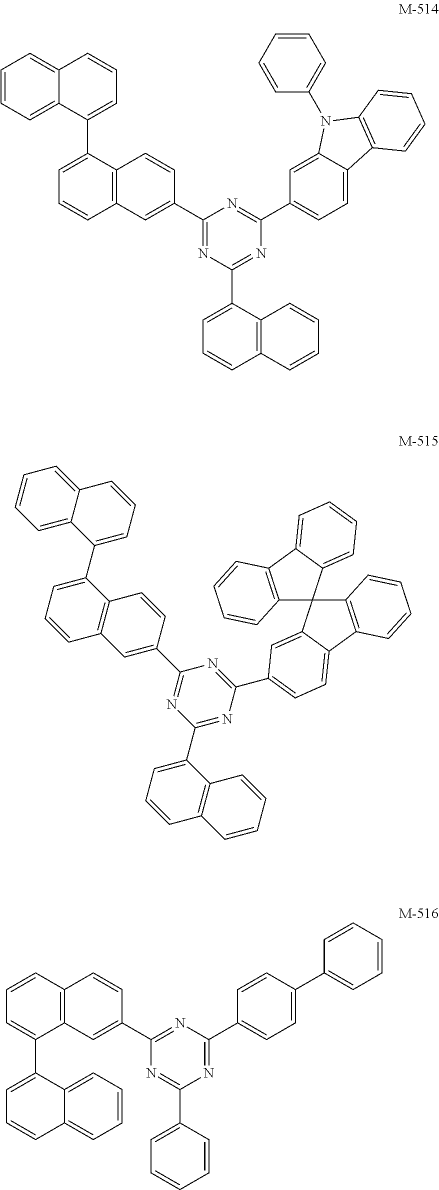 [SMILES: c1ccc(-n2c3ccccc3c3ccc(-c4nc(-c5ccc6c(-c7cccc8ccccc78)cccc6c5)nc(-c5cccc6ccccc56)n4)cc32)cc1 ; c1ccc2c(c1)-c1ccccc1C21c2ccccc2-c2ccc(-c3nc(-c4ccc5c(-c6cccc7ccccc67)cccc5c4)nc(-c4cccc5ccccc45)n3)cc21 ; c1ccc(-c2ccc(-c3nc(-c4ccccc4)nc(-c4ccc5cccc(-c6cccc7ccccc67)c5c4)n3)cc2)cc1]

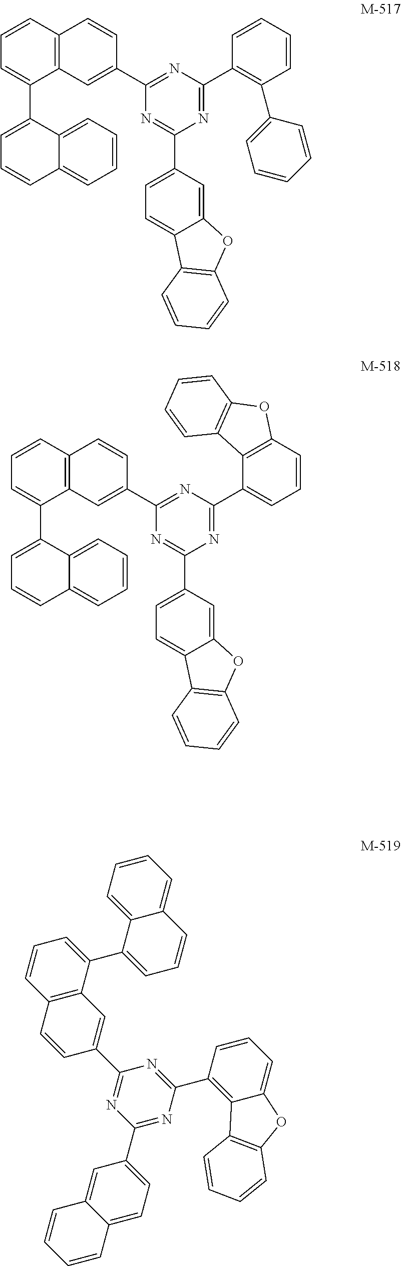 [SMILES: c1ccc(-c2ccccc2-c2nc(-c3ccc4c(c3)oc3ccccc34)nc(-c3ccc4cccc(-c5cccc6ccccc56)c4c3)n2)cc1 ; c1ccc2c(-c3cccc4ccc(-c5nc(-c6ccc7c(c6)oc6ccccc67)nc(-c6cccc7oc8ccccc8c67)n5)cc34)cccc2c1 ; c1ccc2cc(-c3nc(-c4ccc5cccc(-c6cccc7ccccc67)c5c4)nc(-c4cccc5oc6ccccc6c45)n3)ccc2c1]

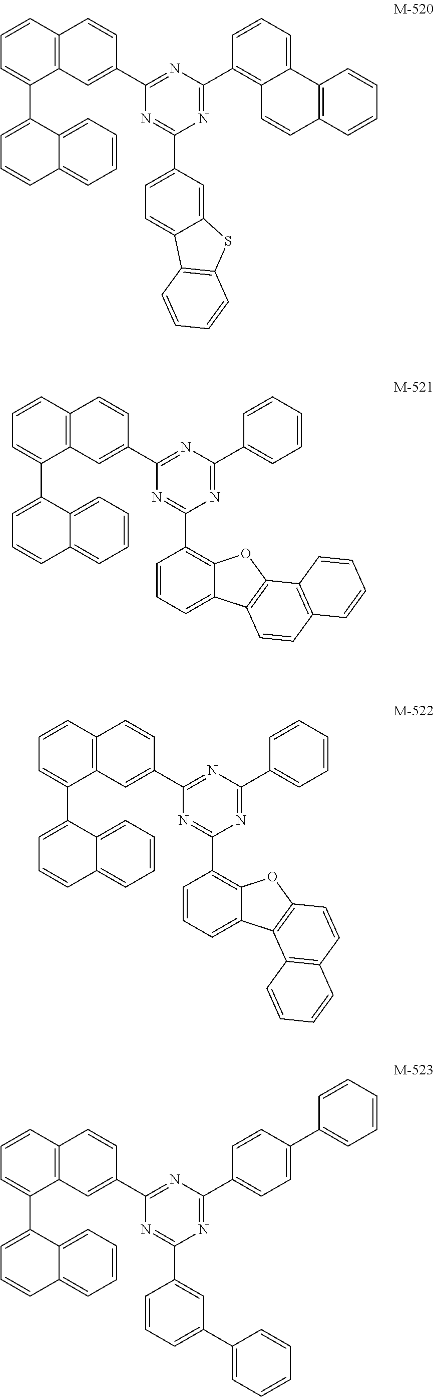 [SMILES: c1ccc2c(-c3cccc4ccc(-c5nc(-c6ccc7c(c6)sc6ccccc67)nc(-c6cccc7c6ccc6ccccc67)n5)cc34)cccc2c1 ; c1ccc(-c2nc(-c3ccc4cccc(-c5cccc6ccccc56)c4c3)nc(-c3cccc4c3oc3c5ccccc5ccc43)n2)cc1 ; c1ccc(-c2nc(-c3ccc4cccc(-c5cccc6ccccc56)c4c3)nc(-c3cccc4c3oc3ccc5ccccc5c34)n2)cc1 ; c1ccc(-c2ccc(-c3nc(-c4cccc(-c5ccccc5)c4)nc(-c4ccc5cccc(-c6cccc7ccccc67)c5c4)n3)cc2)cc1]

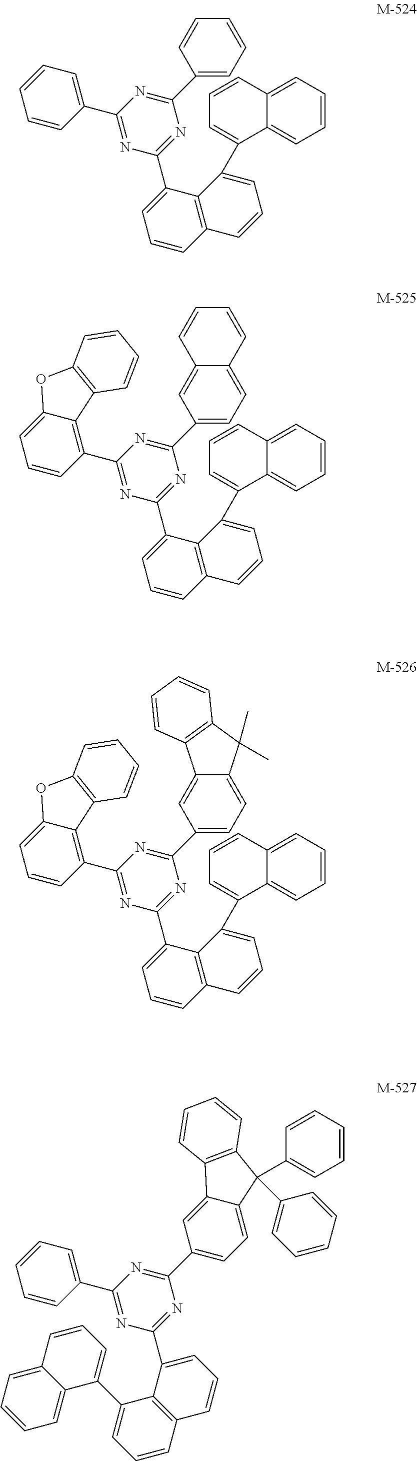 [SMILES: c1ccc(-c2nc(-c3ccccc3)nc(-c3cccc4cccc(-c5cccc6ccccc56)c34)n2)cc1 ; c1ccc2cc(-c3nc(-c4cccc5cccc(-c6cccc7ccccc67)c45)nc(-c4cccc5oc6ccccc6c45)n3)ccc2c1 ; CC1(C)c2ccccc2-c2cc(-c3nc(-c4cccc5cccc(-c6cccc7ccccc67)c45)nc(-c4cccc5oc6ccccc6c45)n3)ccc21 ; c1ccc(-c2nc(-c3ccc4c(c3)-c3ccccc3C4(c3ccccc3)c3ccccc3)nc(-c3cccc4cccc(-c5cccc6ccccc56)c34)n2)cc1]

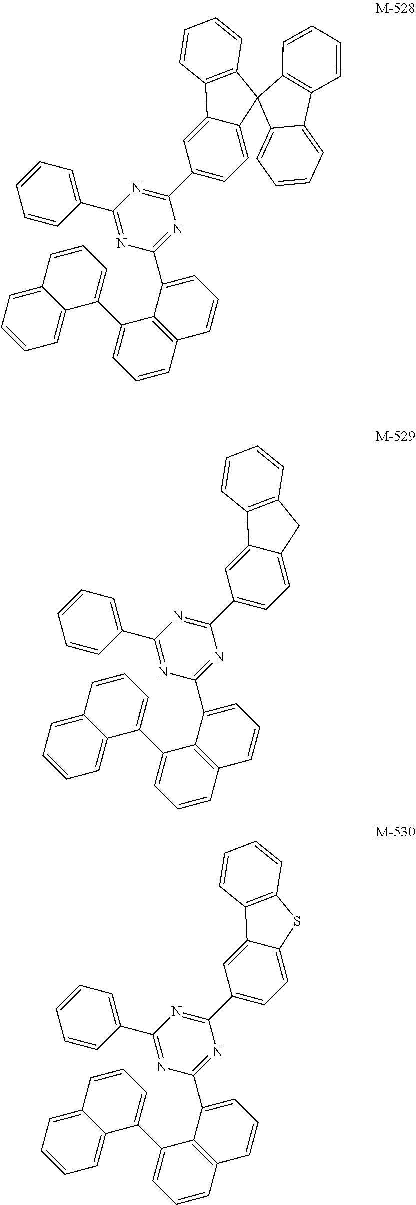 [SMILES: c1ccc(-c2nc(-c3ccc4c(c3)-c3ccccc3C43c4ccccc4-c4ccccc43)nc(-c3cccc4cccc(-c5cccc6ccccc56)c34)n2)cc1 ; c1ccc(-c2nc(-c3ccc4c(c3)-c3ccccc3C4)nc(-c3cccc4cccc(-c5cccc6ccccc56)c34)n2)cc1 ; c1ccc(-c2nc(-c3ccc4sc5ccccc5c4c3)nc(-c3cccc4cccc(-c5cccc6ccccc56)c34)n2)cc1]

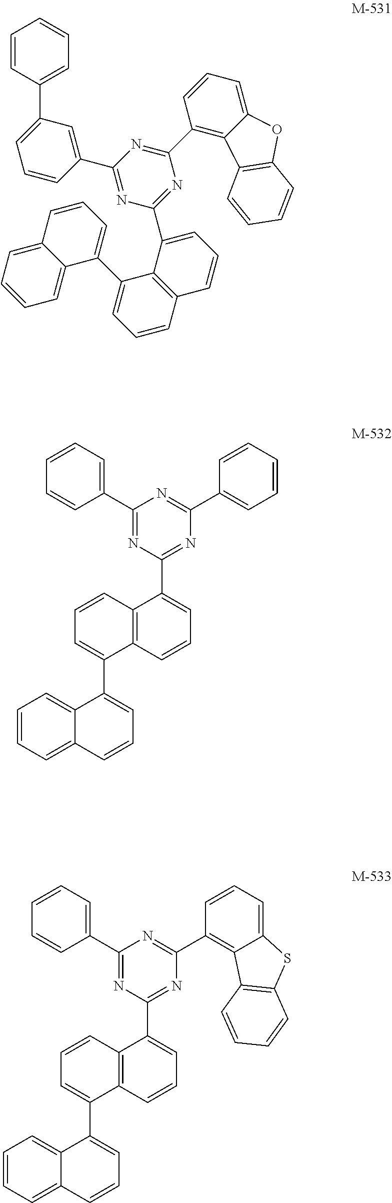 [SMILES: c1ccc(-c2cccc(-c3nc(-c4cccc5cccc(-c6cccc7ccccc67)c45)nc(-c4cccc5oc6ccccc6c45)n3)c2)cc1 ; c1ccc(-c2nc(-c3ccccc3)nc(-c3cccc4c(-c5cccc6ccccc56)cccc34)n2)cc1 ; c1ccc(-c2nc(-c3cccc4c(-c5cccc6ccccc56)cccc34)nc(-c3cccc4sc5ccccc5c34)n2)cc1]

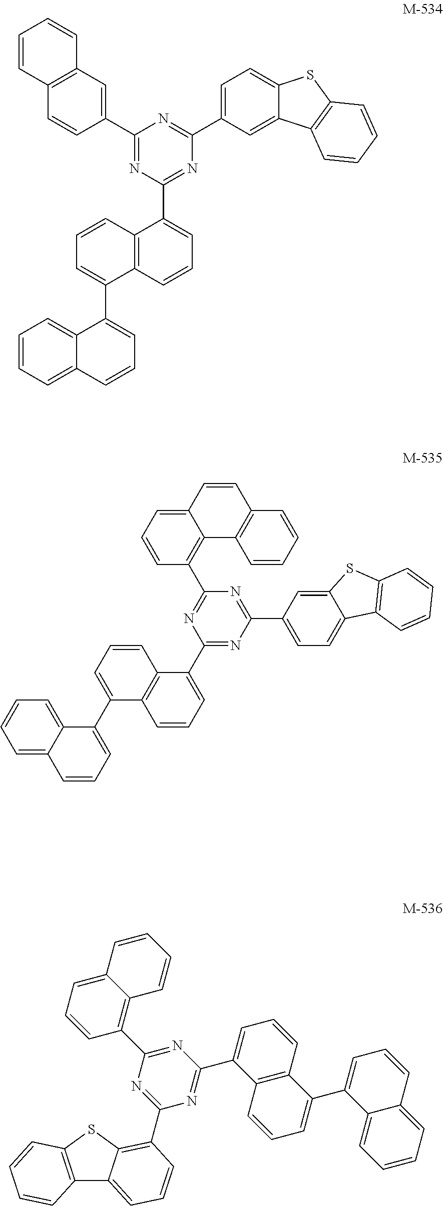 [SMILES: c1ccc2cc(-c3nc(-c4ccc5sc6ccccc6c5c4)nc(-c4cccc5c(-c6cccc7ccccc67)cccc45)n3)ccc2c1 ; c1ccc2c(-c3cccc4c(-c5nc(-c6ccc7c(c6)sc6ccccc67)nc(-c6cccc7ccc8ccccc8c67)n5)cccc34)cccc2c1 ; c1ccc2c(-c3nc(-c4cccc5c(-c6cccc7ccccc67)cccc45)nc(-c4cccc5c4sc4ccccc45)n3)cccc2c1]

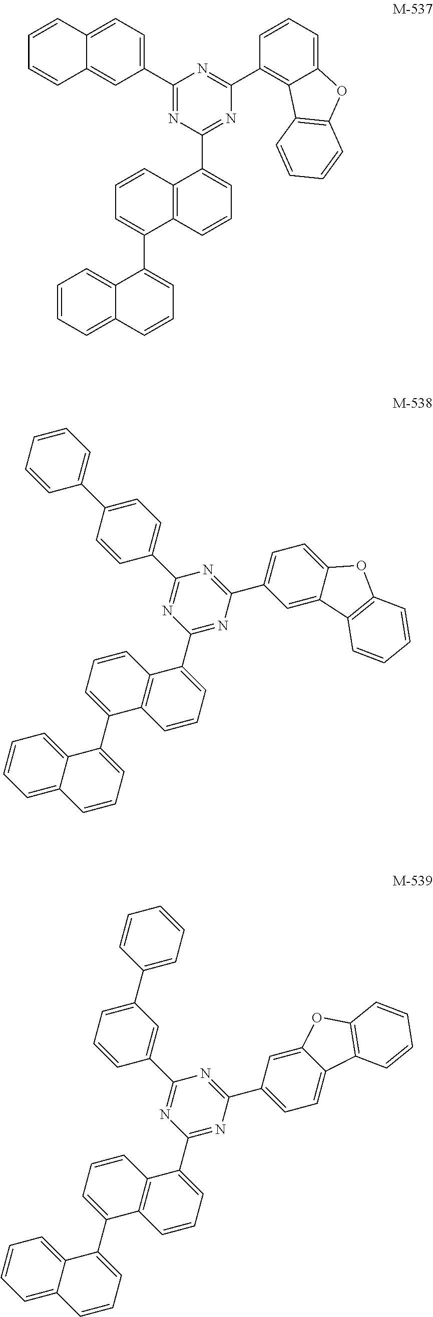 [SMILES: c1ccc2cc(-c3nc(-c4cccc5c(-c6cccc7ccccc67)cccc45)nc(-c4cccc5oc6ccccc6c45)n3)ccc2c1 ; c1ccc(-c2ccc(-c3nc(-c4ccc5oc6ccccc6c5c4)nc(-c4cccc5c(-c6cccc7ccccc67)cccc45)n3)cc2)cc1 ; c1ccc(-c2cccc(-c3nc(-c4ccc5c(c4)oc4ccccc45)nc(-c4cccc5c(-c6cccc7ccccc67)cccc45)n3)c2)cc1]

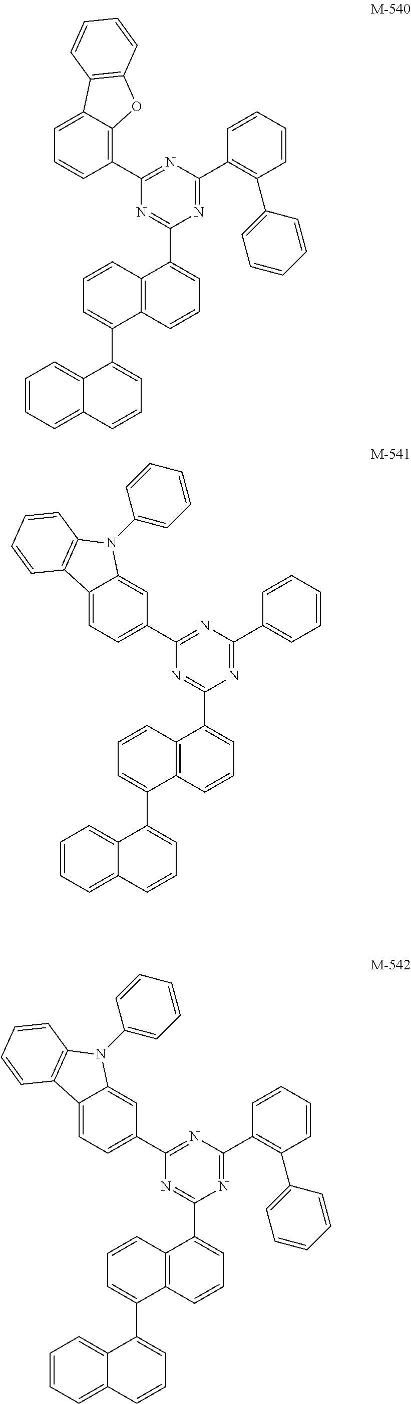 [SMILES: c1ccc(-c2ccccc2-c2nc(-c3cccc4c(-c5cccc6ccccc56)cccc34)nc(-c3cccc4c3oc3ccccc34)n2)cc1 ; c1ccc(-c2nc(-c3ccc4c5ccccc5n(-c5ccccc5)c4c3)nc(-c3cccc4c(-c5cccc6ccccc56)cccc34)n2)cc1 ; c1ccc(-c2ccccc2-c2nc(-c3ccc4c5ccccc5n(-c5ccccc5)c4c3)nc(-c3cccc4c(-c5cccc6ccccc56)cccc34)n2)cc1]

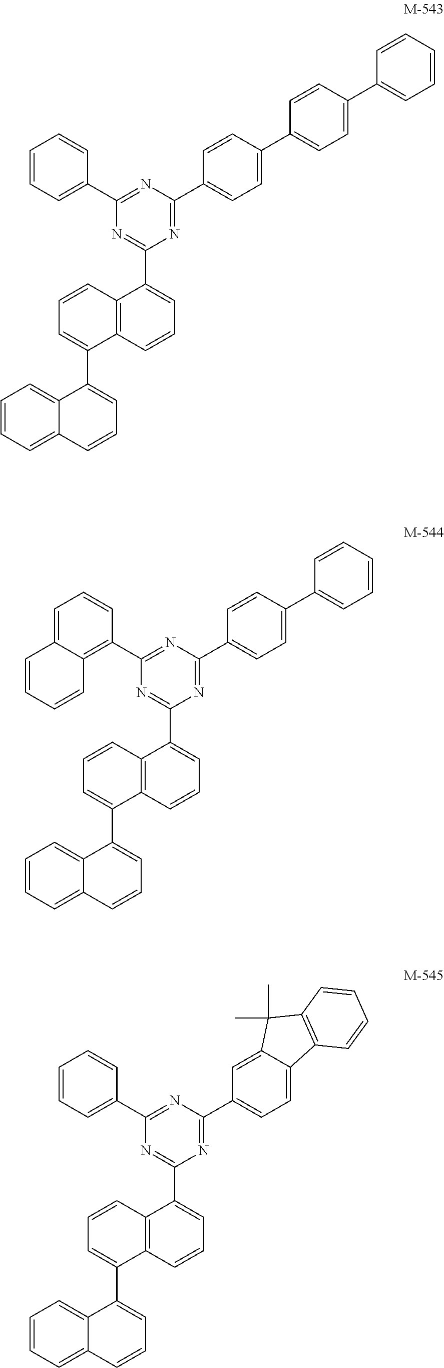 [SMILES: c1ccc(-c2ccc(-c3ccc(-c4nc(-c5ccccc5)nc(-c5cccc6c(-c7cccc8ccccc78)cccc56)n4)cc3)cc2)cc1 ; c1ccc(-c2ccc(-c3nc(-c4cccc5ccccc45)nc(-c4cccc5c(-c6cccc7ccccc67)cccc45)n3)cc2)cc1 ; CC1(C)c2ccccc2-c2ccc(-c3nc(-c4ccccc4)nc(-c4cccc5c(-c6cccc7ccccc67)cccc45)n3)cc21]

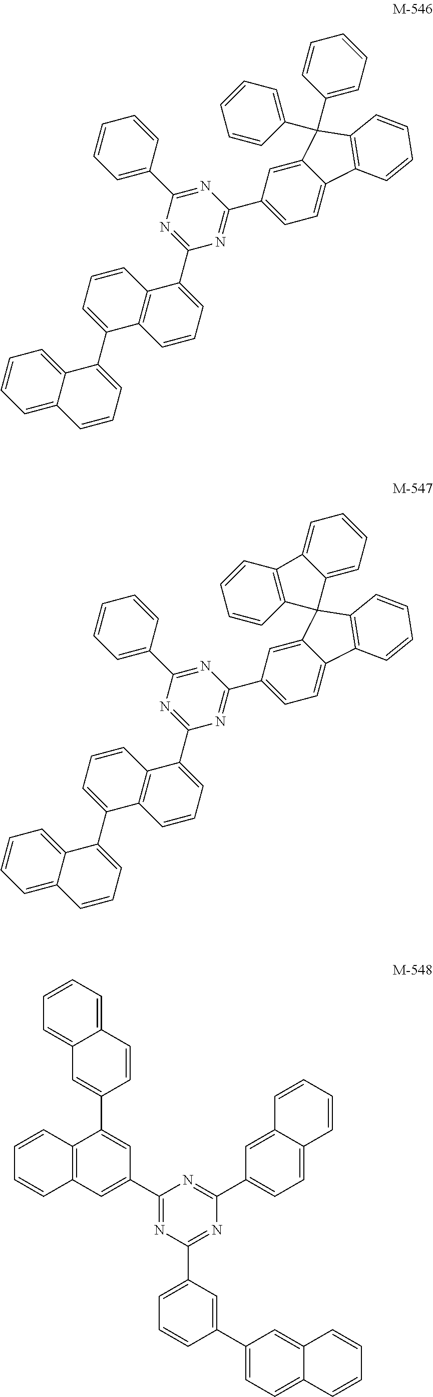 [SMILES: c1ccc(-c2nc(-c3ccc4c(c3)C(c3ccccc3)(c3ccccc3)c3ccccc3-4)nc(-c3cccc4c(-c5cccc6ccccc56)cccc34)n2)cc1 ; c1ccc(-c2nc(-c3ccc4c(c3)C3(c5ccccc5-c5ccccc53)c3ccccc3-4)nc(-c3cccc4c(-c5cccc6ccccc56)cccc34)n2)cc1 ; c1cc(-c2ccc3ccccc3c2)cc(-c2nc(-c3ccc4ccccc4c3)nc(-c3cc(-c4ccc5ccccc5c4)c4ccccc4c3)n2)c1]

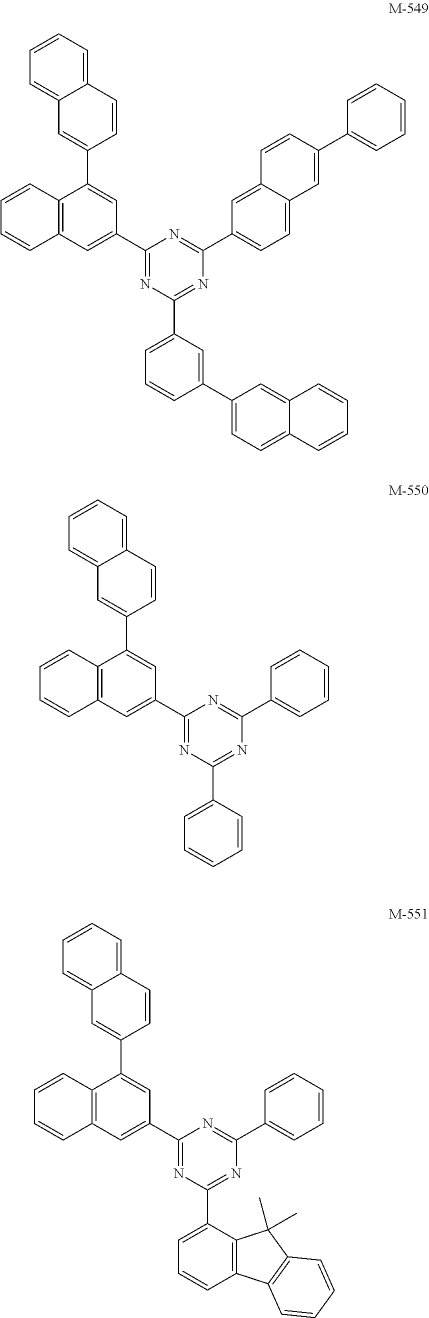 [SMILES: c1ccc(-c2ccc3cc(-c4nc(-c5cccc(-c6ccc7ccccc7c6)c5)nc(-c5cc(-c6ccc7ccccc7c6)c6ccccc6c5)n4)ccc3c2)cc1 ; c1ccc(-c2nc(-c3ccccc3)nc(-c3cc(-c4ccc5ccccc5c4)c4ccccc4c3)n2)cc1 ; CC1(C)c2ccccc2-c2cccc(-c3nc(-c4ccccc4)nc(-c4cc(-c5ccc6ccccc6c5)c5ccccc5c4)n3)c21]

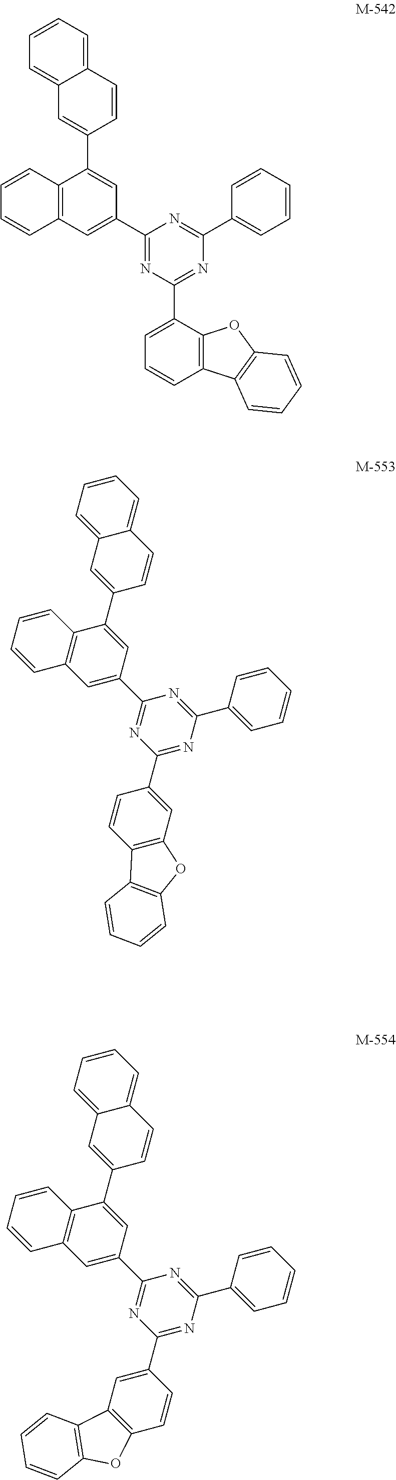 [SMILES: c1ccc(-c2nc(-c3cc(-c4ccc5ccccc5c4)c4ccccc4c3)nc(-c3cccc4c3oc3ccccc34)n2)cc1 ; c1ccc(-c2nc(-c3cc(-c4ccc5ccccc5c4)c4ccccc4c3)nc(-c3ccc4c(c3)oc3ccccc34)n2)cc1 ; c1ccc(-c2nc(-c3cc(-c4ccc5ccccc5c4)c4ccccc4c3)nc(-c3ccc4oc5ccccc5c4c3)n2)cc1]

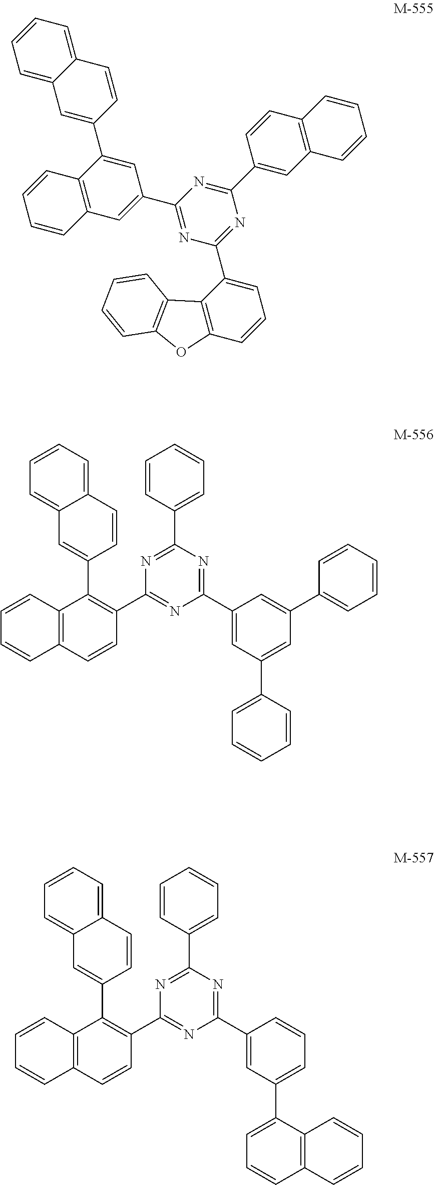 [SMILES: c1ccc2cc(-c3nc(-c4cc(-c5ccc6ccccc6c5)c5ccccc5c4)nc(-c4cccc5oc6ccccc6c45)n3)ccc2c1 ; c1ccc(-c2cc(-c3ccccc3)cc(-c3nc(-c4ccccc4)nc(-c4ccc5ccccc5c4-c4ccc5ccccc5c4)n3)c2)cc1 ; c1ccc(-c2nc(-c3cccc(-c4cccc5ccccc45)c3)nc(-c3ccc4ccccc4c3-c3ccc4ccccc4c3)n2)cc1]

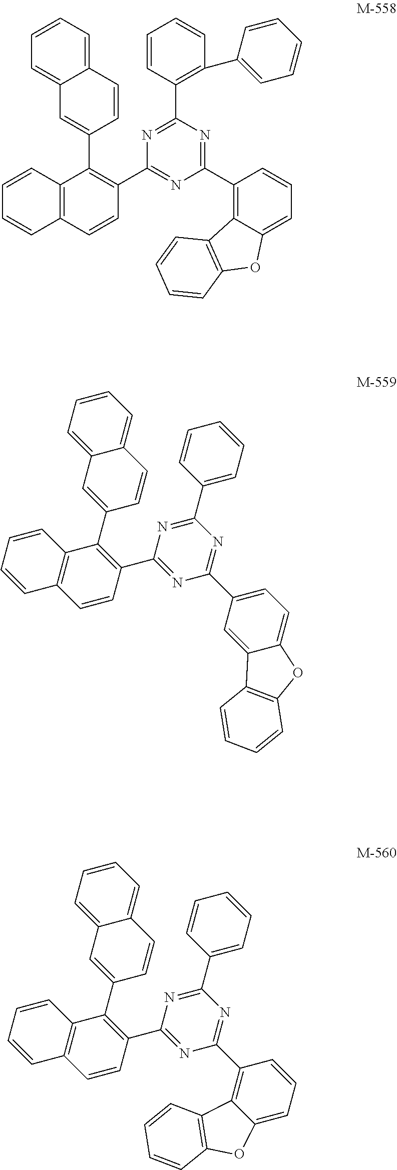 [SMILES: c1ccc(-c2ccccc2-c2nc(-c3ccc4ccccc4c3-c3ccc4ccccc4c3)nc(-c3cccc4oc5ccccc5c34)n2)cc1 ; c1ccc(-c2nc(-c3ccc4oc5ccccc5c4c3)nc(-c3ccc4ccccc4c3-c3ccc4ccccc4c3)n2)cc1 ; c1ccc(-c2nc(-c3ccc4ccccc4c3-c3ccc4ccccc4c3)nc(-c3cccc4oc5ccccc5c34)n2)cc1]

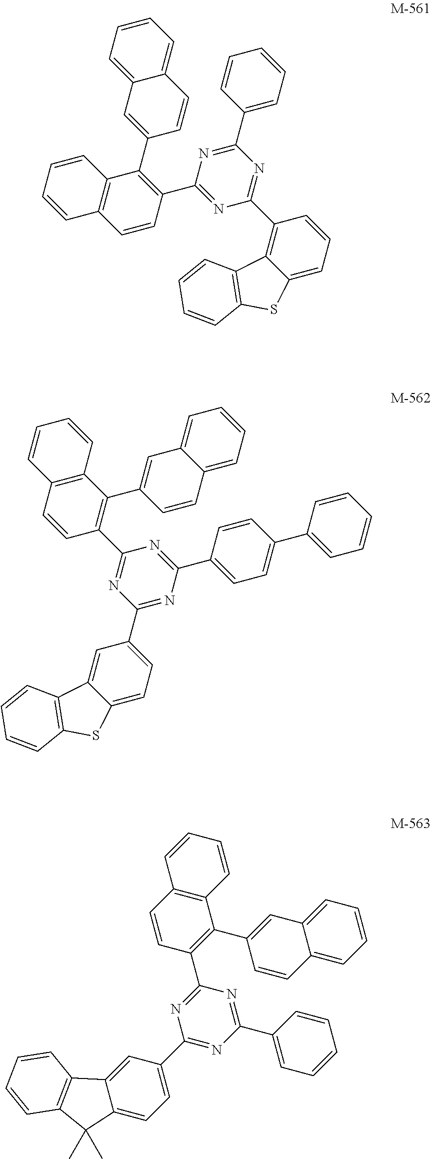 [SMILES: c1ccc(-c2nc(-c3ccc4ccccc4c3-c3ccc4ccccc4c3)nc(-c3cccc4sc5ccccc5c34)n2)cc1 ; c1ccc(-c2ccc(-c3nc(-c4ccc5sc6ccccc6c5c4)nc(-c4ccc5ccccc5c4-c4ccc5ccccc5c4)n3)cc2)cc1 ; CC1(C)c2ccccc2-c2cc(-c3nc(-c4ccccc4)nc(-c4ccc5ccccc5c4-c4ccc5ccccc5c4)n3)ccc21]

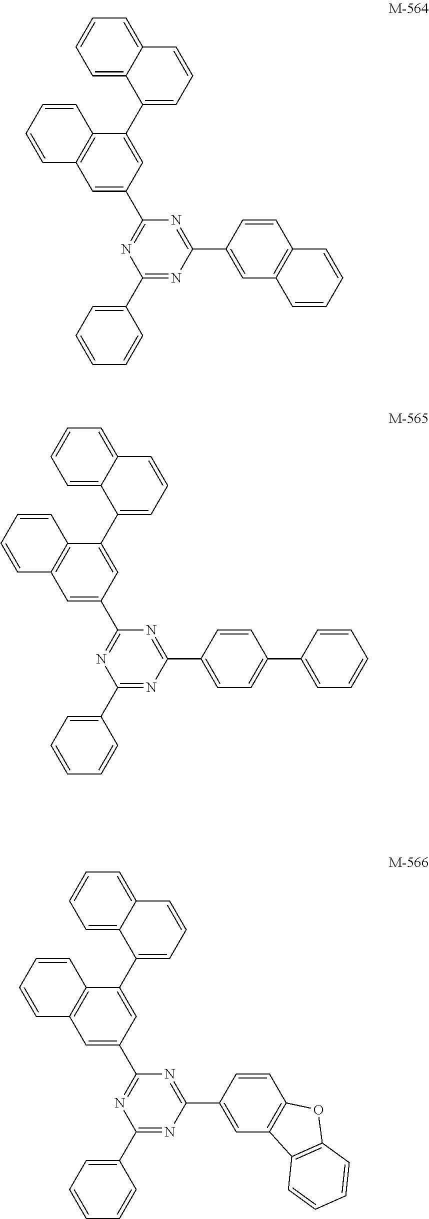 [SMILES: c1ccc(-c2nc(-c3ccc4ccccc4c3)nc(-c3cc(-c4cccc5ccccc45)c4ccccc4c3)n2)cc1 ; c1ccc(-c2ccc(-c3nc(-c4ccccc4)nc(-c4cc(-c5cccc6ccccc56)c5ccccc5c4)n3)cc2)cc1 ; c1ccc(-c2nc(-c3cc(-c4cccc5ccccc45)c4ccccc4c3)nc(-c3ccc4oc5ccccc5c4c3)n2)cc1]

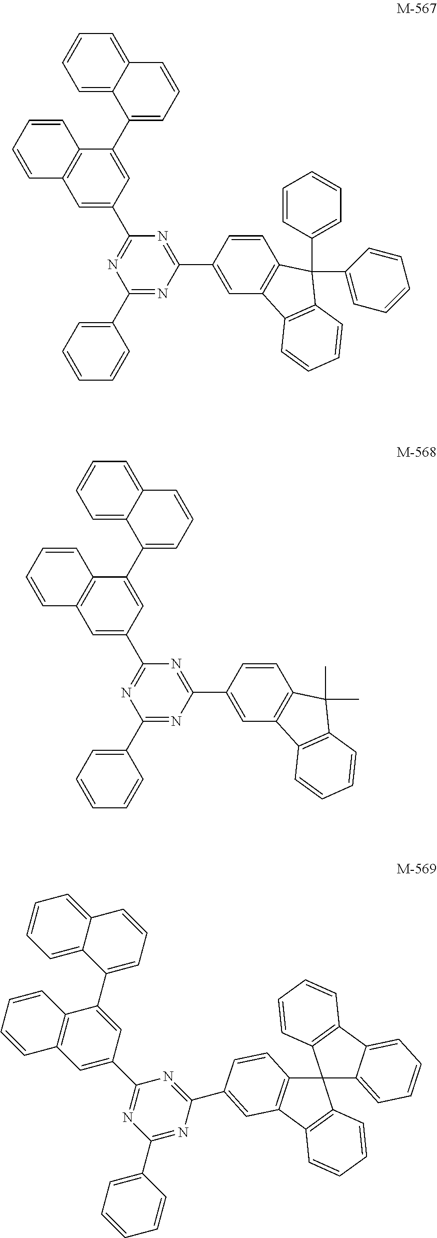 [SMILES: c1ccc(-c2nc(-c3ccc4c(c3)-c3ccccc3C4(c3ccccc3)c3ccccc3)nc(-c3cc(-c4cccc5ccccc45)c4ccccc4c3)n2)cc1 ; CC1(C)c2ccccc2-c2cc(-c3nc(-c4ccccc4)nc(-c4cc(-c5cccc6ccccc56)c5ccccc5c4)n3)ccc21 ; c1ccc(-c2nc(-c3ccc4c(c3)-c3ccccc3C43c4ccccc4-c4ccccc43)nc(-c3cc(-c4cccc5ccccc45)c4ccccc4c3)n2)cc1]

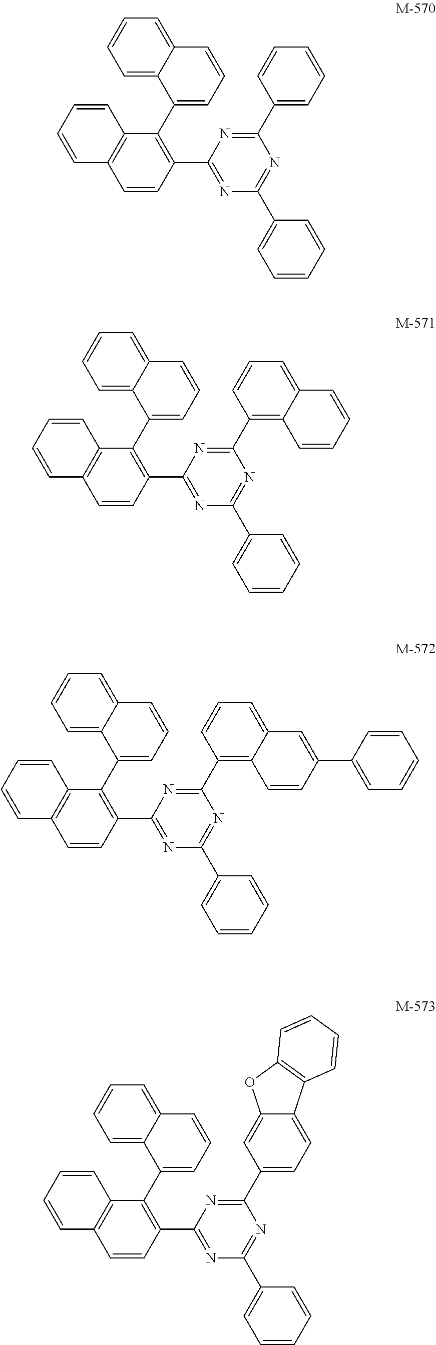 [SMILES: c1ccc(-c2nc(-c3ccccc3)nc(-c3ccc4ccccc4c3-c3cccc4ccccc34)n2)cc1 ; c1ccc(-c2nc(-c3ccc4ccccc4c3-c3cccc4ccccc34)nc(-c3cccc4ccccc34)n2)cc1 ; c1ccc(-c2ccc3c(-c4nc(-c5ccccc5)nc(-c5ccc6ccccc6c5-c5cccc6ccccc56)n4)cccc3c2)cc1 ; c1ccc(-c2nc(-c3ccc4c(c3)oc3ccccc34)nc(-c3ccc4ccccc4c3-c3cccc4ccccc34)n2)cc1]

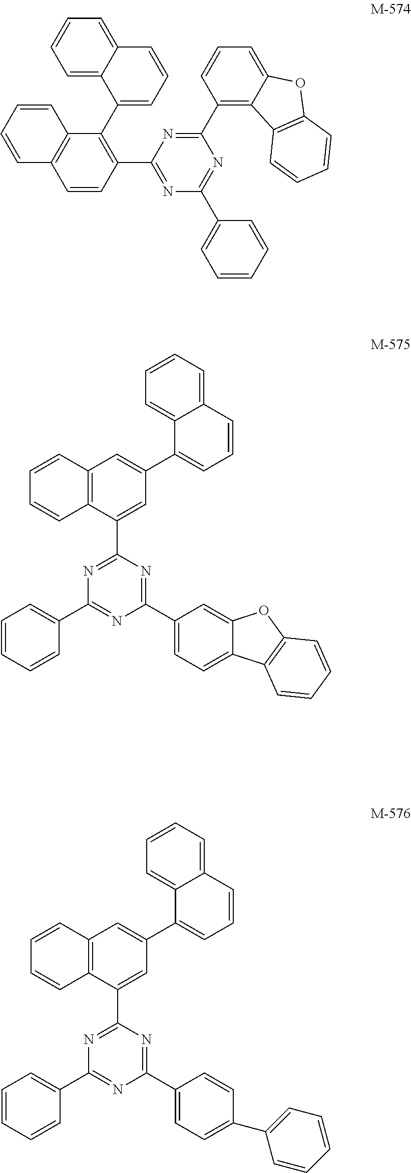 [SMILES: c1ccc(-c2nc(-c3ccc4ccccc4c3-c3cccc4ccccc34)nc(-c3cccc4oc5ccccc5c34)n2)cc1 ; c1ccc(-c2nc(-c3ccc4c(c3)oc3ccccc34)nc(-c3cc(-c4cccc5ccccc45)cc4ccccc34)n2)cc1 ; c1ccc(-c2ccc(-c3nc(-c4ccccc4)nc(-c4cc(-c5cccc6ccccc56)cc5ccccc45)n3)cc2)cc1]

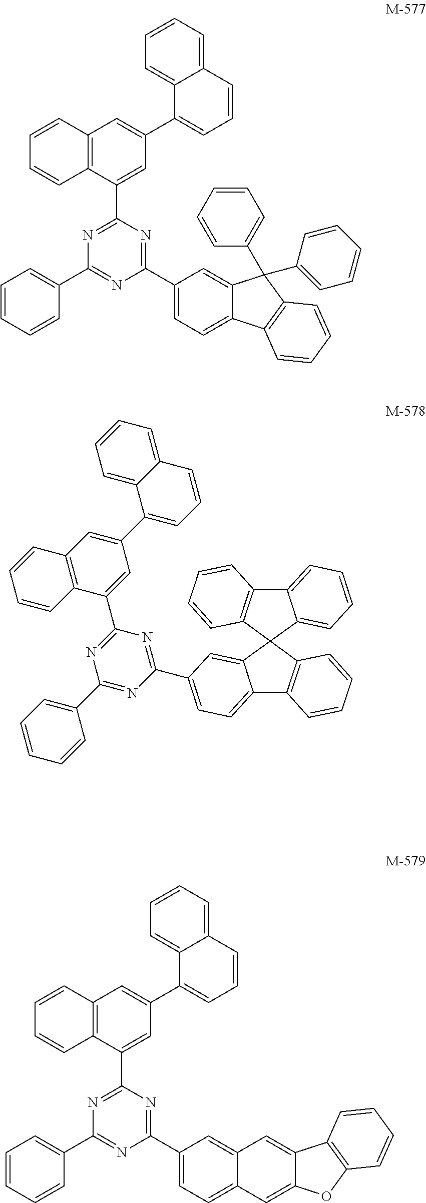 [SMILES: c1ccc(-c2nc(-c3ccc4c(c3)C(c3ccccc3)(c3ccccc3)c3ccccc3-4)nc(-c3cc(-c4cccc5ccccc45)cc4ccccc34)n2)cc1 ; c1ccc(-c2nc(-c3ccc4c(c3)C3(c5ccccc5-c5ccccc53)c3ccccc3-4)nc(-c3cc(-c4cccc5ccccc45)cc4ccccc34)n2)cc1 ; c1ccc(-c2nc(-c3ccc4cc5oc6ccccc6c5cc4c3)nc(-c3cc(-c4cccc5ccccc45)cc4ccccc34)n2)cc1]

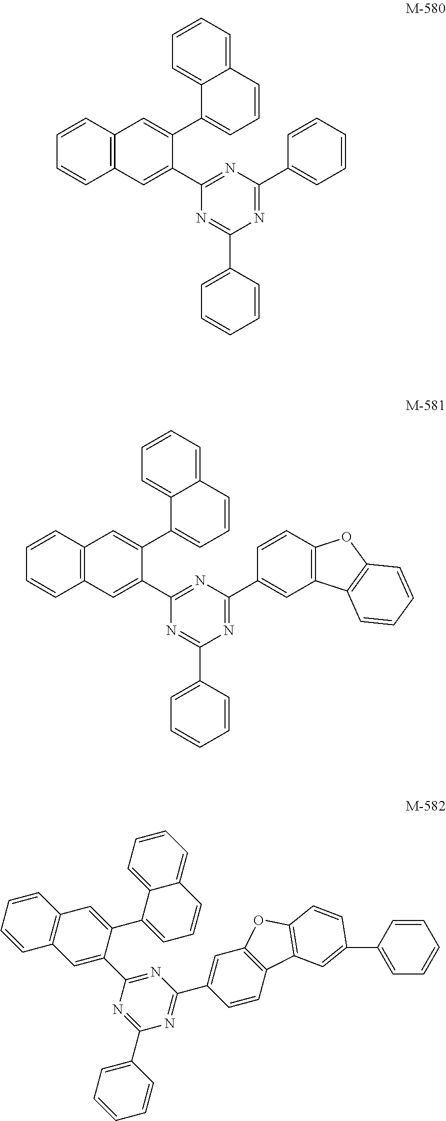 [SMILES: c1ccc(-c2nc(-c3ccccc3)nc(-c3cc4ccccc4cc3-c3cccc4ccccc34)n2)cc1 ; c1ccc(-c2nc(-c3ccc4oc5ccccc5c4c3)nc(-c3cc4ccccc4cc3-c3cccc4ccccc34)n2)cc1 ; c1ccc(-c2ccc3oc4cc(-c5nc(-c6ccccc6)nc(-c6cc7ccccc7cc6-c6cccc7ccccc67)n5)ccc4c3c2)cc1]

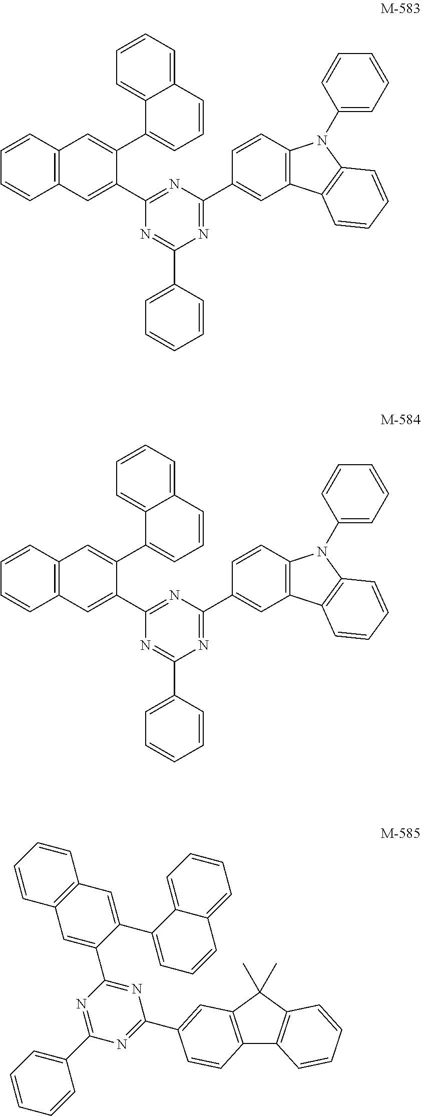 [SMILES: c1ccc(-c2nc(-c3ccc4c(c3)c3ccccc3n4-c3ccccc3)nc(-c3cc4ccccc4cc3-c3cccc4ccccc34)n2)cc1 ; c1ccc(-c2nc(-c3ccc4c(c3)c3ccccc3n4-c3ccccc3)nc(-c3cc4ccccc4cc3-c3cccc4ccccc34)n2)cc1 ; CC1(C)c2ccccc2-c2ccc(-c3nc(-c4ccccc4)nc(-c4cc5ccccc5cc4-c4cccc5ccccc45)n3)cc21]

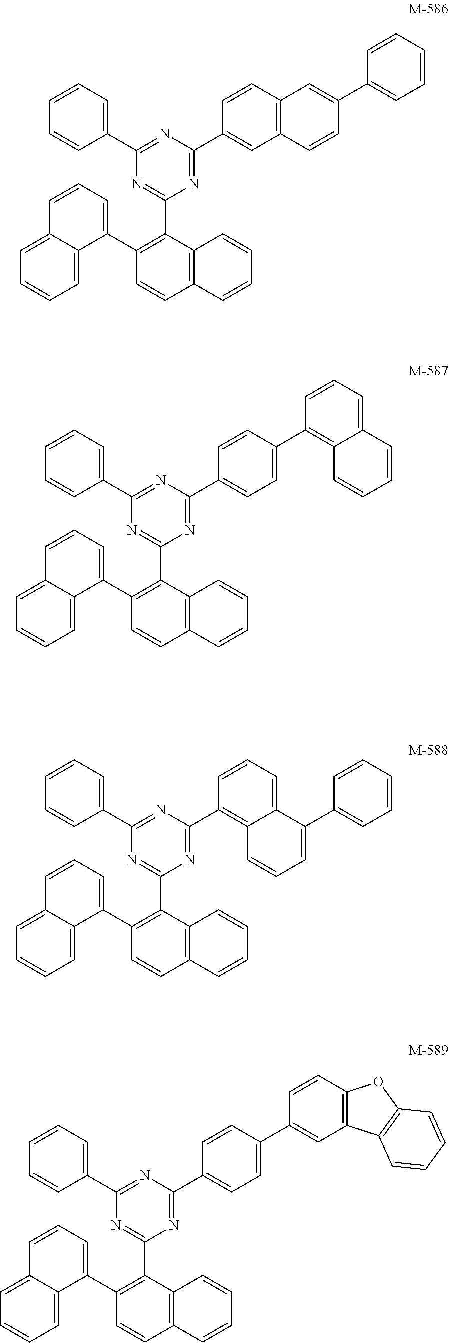 [SMILES: c1ccc(-c2ccc3cc(-c4nc(-c5ccccc5)nc(-c5c(-c6cccc7ccccc67)ccc6ccccc56)n4)ccc3c2)cc1 ; c1ccc(-c2nc(-c3ccc(-c4cccc5ccccc45)cc3)nc(-c3c(-c4cccc5ccccc45)ccc4ccccc34)n2)cc1 ; c1ccc(-c2nc(-c3c(-c4cccc5ccccc45)ccc4ccccc34)nc(-c3cccc4c(-c5ccccc5)cccc34)n2)cc1 ; c1ccc(-c2nc(-c3ccc(-c4ccc5oc6ccccc6c5c4)cc3)nc(-c3c(-c4cccc5ccccc45)ccc4ccccc34)n2)cc1]

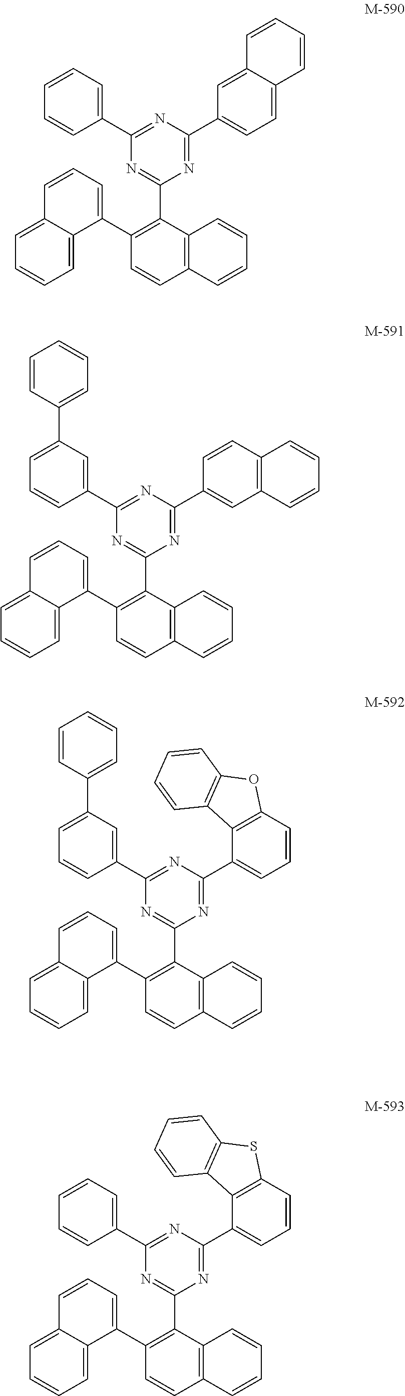 [SMILES: c1ccc(-c2nc(-c3ccc4ccccc4c3)nc(-c3c(-c4cccc5ccccc45)ccc4ccccc34)n2)cc1 ; c1ccc(-c2cccc(-c3nc(-c4ccc5ccccc5c4)nc(-c4c(-c5cccc6ccccc56)ccc5ccccc45)n3)c2)cc1 ; c1ccc(-c2cccc(-c3nc(-c4c(-c5cccc6ccccc56)ccc5ccccc45)nc(-c4cccc5oc6ccccc6c45)n3)c2)cc1 ; c1ccc(-c2nc(-c3c(-c4cccc5ccccc45)ccc4ccccc34)nc(-c3cccc4sc5ccccc5c34)n2)cc1]

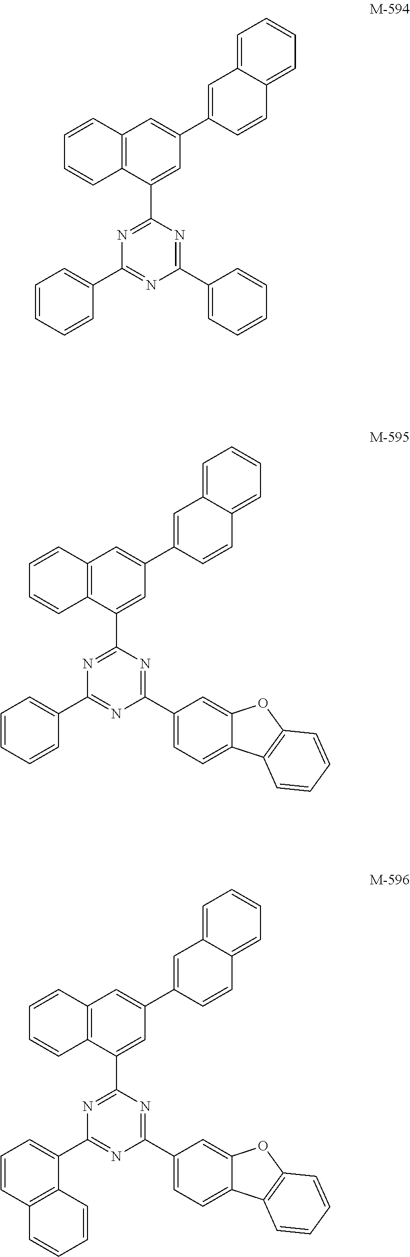 [SMILES: c1ccc(-c2nc(-c3ccccc3)nc(-c3cc(-c4ccc5ccccc5c4)cc4ccccc34)n2)cc1 ; c1ccc(-c2nc(-c3ccc4c(c3)oc3ccccc34)nc(-c3cc(-c4ccc5ccccc5c4)cc4ccccc34)n2)cc1 ; c1ccc2cc(-c3cc(-c4nc(-c5ccc6c(c5)oc5ccccc56)nc(-c5cccc6ccccc56)n4)c4ccccc4c3)ccc2c1]

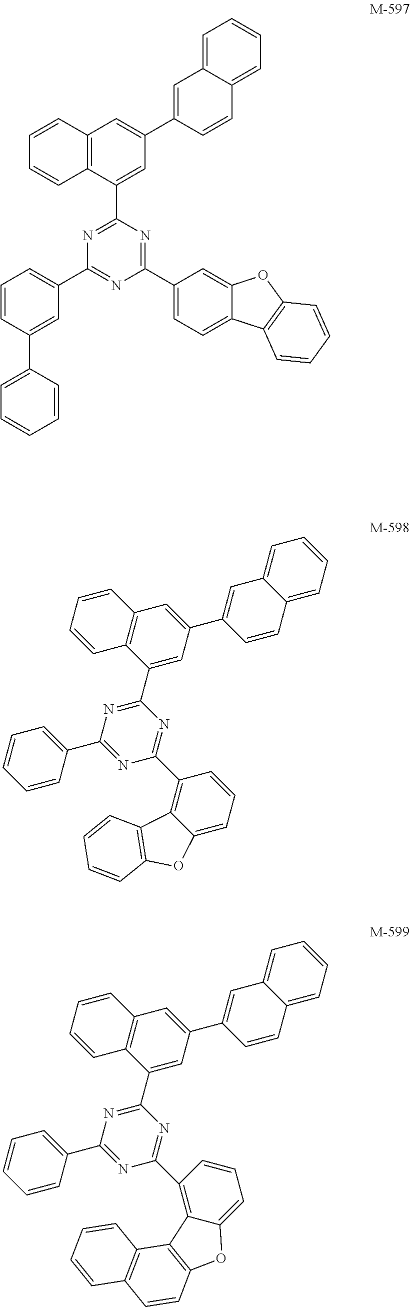 [SMILES: c1ccc(-c2cccc(-c3nc(-c4ccc5c(c4)oc4ccccc45)nc(-c4cc(-c5ccc6ccccc6c5)cc5ccccc45)n3)c2)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cc(-c4ccc5ccccc5c4)cc4ccccc34)nc(-c3cccc4oc5ccc6ccccc6c5c34)n2)cc1]

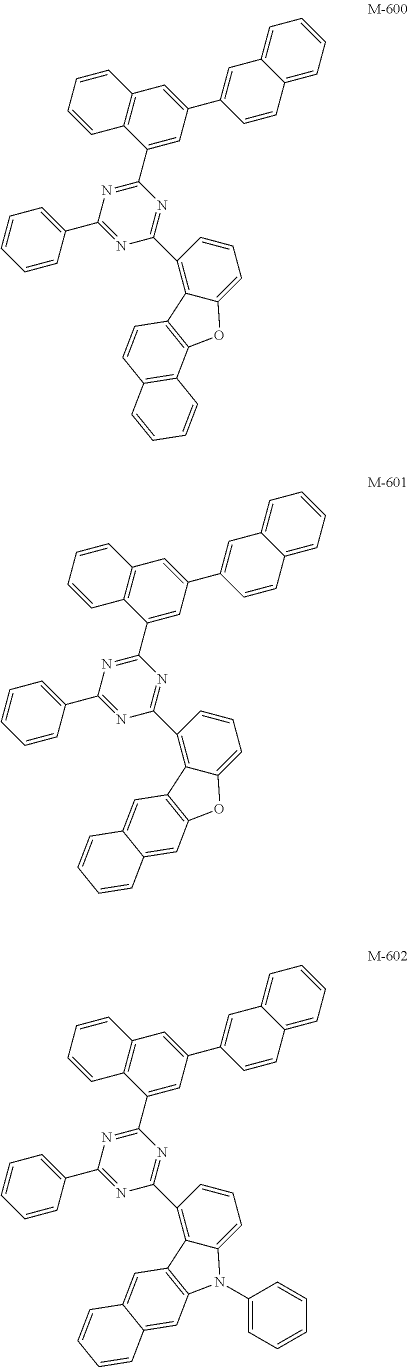 [SMILES: c1ccc(-c2nc(-c3cc(-c4ccc5ccccc5c4)cc4ccccc34)nc(-c3cccc4oc5c6ccccc6ccc5c34)n2)cc1 ; c1ccc(-c2nc(-c3cc(-c4ccc5ccccc5c4)cc4ccccc34)nc(-c3cccc4oc5cc6ccccc6cc5c34)n2)cc1 ; c1ccc(-c2nc(-c3cc(-c4ccc5ccccc5c4)cc4ccccc34)nc(-c3cccc4c3c3cc5ccccc5cc3n4-c3ccccc3)n2)cc1]

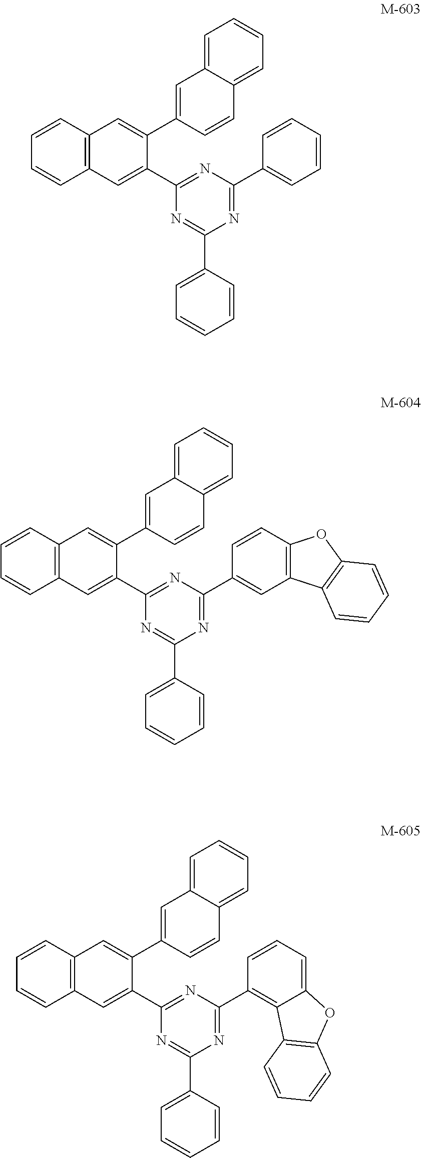 [SMILES: c1ccc(-c2nc(-c3ccccc3)nc(-c3cc4ccccc4cc3-c3ccc4ccccc4c3)n2)cc1 ; c1ccc(-c2nc(-c3ccc4oc5ccccc5c4c3)nc(-c3cc4ccccc4cc3-c3ccc4ccccc4c3)n2)cc1 ; c1ccc(-c2nc(-c3cc4ccccc4cc3-c3ccc4ccccc4c3)nc(-c3cccc4oc5ccccc5c34)n2)cc1]

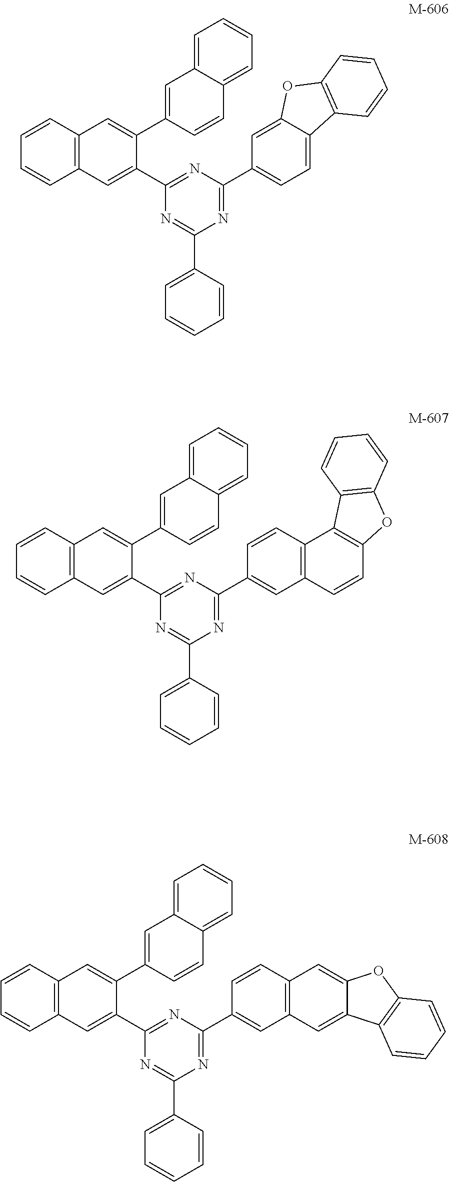 [SMILES: c1ccc(-c2nc(-c3ccc4c(c3)oc3ccccc34)nc(-c3cc4ccccc4cc3-c3ccc4ccccc4c3)n2)cc1 ; c1ccc(-c2nc(-c3ccc4c(ccc5oc6ccccc6c54)c3)nc(-c3cc4ccccc4cc3-c3ccc4ccccc4c3)n2)cc1 ; c1ccc(-c2nc(-c3ccc4cc5oc6ccccc6c5cc4c3)nc(-c3cc4ccccc4cc3-c3ccc4ccccc4c3)n2)cc1]

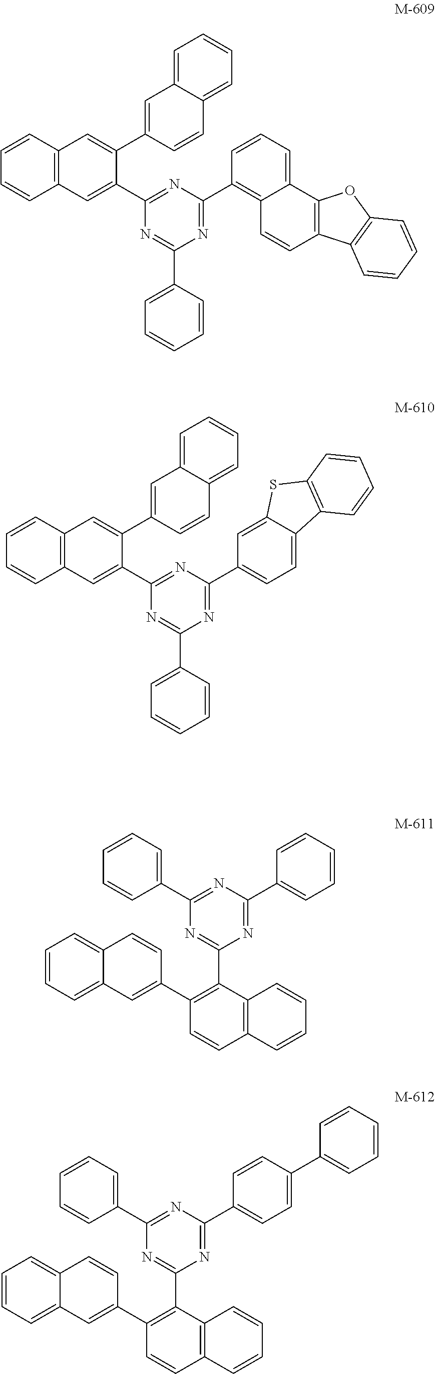 [SMILES: c1ccc(-c2nc(-c3cc4ccccc4cc3-c3ccc4ccccc4c3)nc(-c3cccc4c3ccc3c5ccccc5oc43)n2)cc1 ; c1ccc(-c2nc(-c3ccc4c(c3)sc3ccccc34)nc(-c3cc4ccccc4cc3-c3ccc4ccccc4c3)n2)cc1 ; c1ccc(-c2nc(-c3ccccc3)nc(-c3c(-c4ccc5ccccc5c4)ccc4ccccc34)n2)cc1 ; c1ccc(-c2ccc(-c3nc(-c4ccccc4)nc(-c4c(-c5ccc6ccccc6c5)ccc5ccccc45)n3)cc2)cc1]

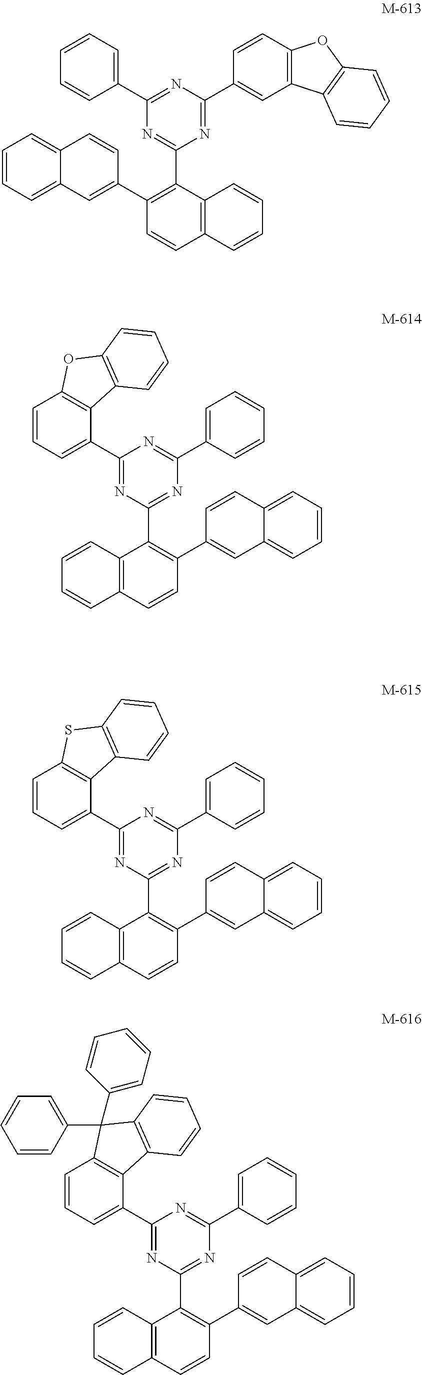 [SMILES: c1ccc(-c2nc(-c3ccc4oc5ccccc5c4c3)nc(-c3c(-c4ccc5ccccc5c4)ccc4ccccc34)n2)cc1 ; c1ccc(-c2nc(-c3c(-c4ccc5ccccc5c4)ccc4ccccc34)nc(-c3cccc4oc5ccccc5c34)n2)cc1 ; c1ccc(-c2nc(-c3c(-c4ccc5ccccc5c4)ccc4ccccc34)nc(-c3cccc4sc5ccccc5c34)n2)cc1 ; c1ccc(-c2nc(-c3cccc4c3-c3ccccc3C4(c3ccccc3)c3ccccc3)nc(-c3c(-c4ccc5ccccc5c4)ccc4ccccc34)n2)cc1]

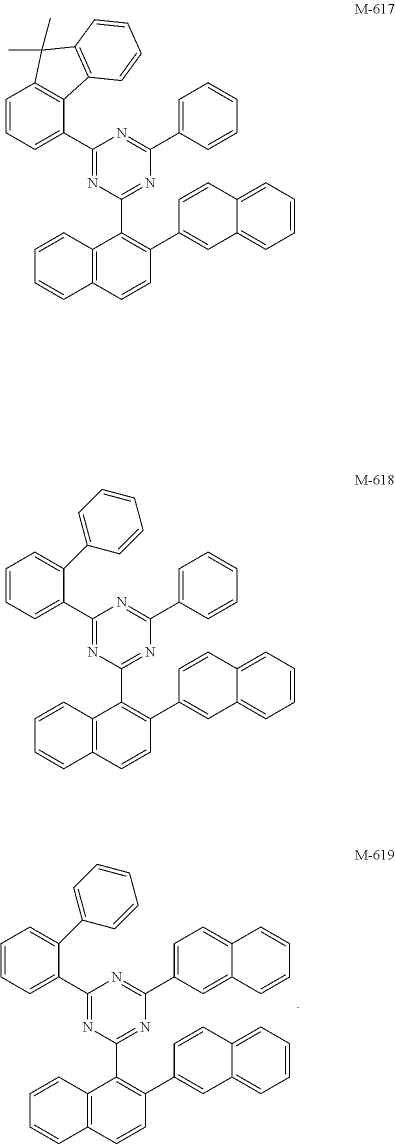 [SMILES: CC1(C)c2ccccc2-c2c(-c3nc(-c4ccccc4)nc(-c4c(-c5ccc6ccccc6c5)ccc5ccccc45)n3)cccc21 ; c1ccc(-c2nc(-c3ccccc3-c3ccccc3)nc(-c3c(-c4ccc5ccccc5c4)ccc4ccccc34)n2)cc1 ; c1ccc(-c2ccccc2-c2nc(-c3ccc4ccccc4c3)nc(-c3c(-c4ccc5ccccc5c4)ccc4ccccc34)n2)cc1]

8. An organic electroluminescent host material composition, comprising a compound N having a structure shown in Formula (1) and the compound M having the structure shown in Formula (2) according to claim 1; wherein
Formula (1): 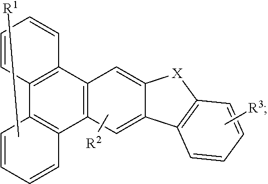
in Formula (1), X is selected from O, S, Se, NAr and CR⁶R⁷; wherein Ar is selected from hydrogen, deuterium, halogen, cyano, substituted or unsubstituted C1-C30 alkyl, substituted or unsubstituted C3-C30 cycloalkyl, substituted or unsubstituted C5-C60 aryl, and substituted or unsubstituted C3-C60 heteroaryl; R⁶-R⁷ are each independently selected from hydrogen atoms, substituted or unsubstituted C1-C20 alkyl, substituted or unsubstituted C6-C30 aryl, and substituted or unsubstituted C3-C30 heteroaryl;
R¹ is -L¹Ar¹, R² is -L²Ar², and R³ is -L³Ar³; L¹-L³ are each independently selected from a connecting bond, substituted or unsubstituted C6-C30 arylenyl, and substituted or unsubstituted C3-C30 heteroarylenyl; at least one of Ar¹-Ar³ is 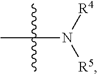
and [symbol] represents a connecting bond, wherein R⁴ is -L⁴Ar⁴, R⁵ is -L⁵Ar⁵, L⁴-L⁵ are each independently selected from a connecting bond, substituted or unsubstituted C6-C30 arylenyl and substituted or unsubstituted C3-C30 heteroarylenyl, and Ar⁴-Ar⁵ are each independently selected from hydrogen, substituted or unsubstituted C6-C30 aryl and substituted or unsubstituted C3-C30 heteroaryl; and the remaining of Ar¹-Ar³ are each independently selected from hydrogen, deuterium, tritium, halogen, cyano, substituted or unsubstituted C6-C60 arylamine group, substituted or unsubstituted C3-C60 heteroarylamine group, substituted or unsubstituted C6-C60 aryl, and substituted or unsubstituted C3-C60 heteroaryl.

9. The composition according to claim 8, wherein Ar⁴-Ar⁵ are each independently selected from the following substituted or unsubstituted groups: phenyl, naphthyl, biphenyl, triphenyl, phenanthryl, triphenylenyl, chrysenyl, dibenzofuryl, benzonaphthofuryl, dibenzothienyl, dibenzoselenophenyl, triphenylenyl, dimethylfluorenyl, spirobifluorenyl, fluoranthenyl, carbazolyl, phenylcarbazolyl, diphenylfluorenyl, benzodimethylfluorenyl, benzodiphenylfluorenyl, benzospirobifluorenyl, pyridyl, pyrimidinyl and triazinyl.

10. The composition according to claim 8, wherein Ar⁴-Ar⁵ are each independently selected from 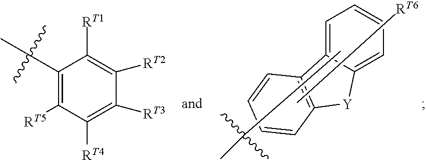
wherein, R^{T1}-R^{T6} are each independently selected from hydrogen, deuterium, halogen, cyano, substituted or unsubstituted C1-C30 alkyl, substituted or unsubstituted C7-C30 aralkyl, substituted or unsubstituted C6-C30 aryl, substituted or unsubstituted C3-C30 heteroaryl, substituted or unsubstituted C4-C30 heteroaralkyl, substituted or unsubstituted C3-C30 cycloalkyl, substituted or unsubstituted C3-C30 heterocycloalkyl, substituted or unsubstituted C3-C30 cycloalkenyl, substituted or unsubstituted C1-C30 alkoxy, and substituted or unsubstituted C6-C30 aryloxy, or any two adjacent ones of R^{T1}-R^{T5} are capable of being fused to a C6-C30 ring A; Y is selected from O, S, NAr and CR⁶R⁷; wherein Ar is selected from hydrogen, deuterium, halogen, cyano, substituted or unsubstituted C1-C30 alkyl, substituted or unsubstituted C3-C30 cycloalkyl, substituted or unsubstituted C5-C60 aryl, and substituted or unsubstituted C3-C60 heteroaryl; R⁶-R⁷ are each independently selected from hydrogen atoms, substituted or unsubstituted C1-C20 alkyl, substituted or unsubstituted C6-C30 aryl, and substituted or unsubstituted C3-C30 heteroaryl;
when a plurality of R^{T1}-R^{T6} are present, R^{T1}-R^{T6} are independent of each other and may be the same or different; and
preferably, ring A is selected from a substituted or unsubstituted benzene ring, a substituted or unsubstituted naphthalene ring, and a substituted or unsubstituted phenanthrene ring.

11. The composition according to claim 8, wherein Ar is selected from substituted or unsubstituted C6-C30 aryl and C3-C30 heteroaryl; preferably, Ar is selected from the following substituted or unsubstituted groups: phenyl, naphthyl, biphenyl, triphenyl, triphenylenyl, chrysenyl, dibenzofuryl and dibenzothienyl;
and/or, R⁶ and R⁷ are each independently selected from substituted or unsubstituted C1-C5 alkyl and substituted or unsubstituted C6-C30 aryl.

12. The composition according to claim 8, wherein L¹-L³ are each independently selected from a connecting bond and C6-C30 arylenyl, and preferably, L¹-L³ are each independently selected from a connecting bond, phenylenyl, naphthylenyl, triphenylenyl and biphenylenyl; further optionally, L¹ is a connecting bond, L² is selected from phenylenyl, naphthylenyl, triphenylenyl and biphenylenyl, and L³ is a connecting bond; preferably, L¹-L³ are each independently selected from a single bond;
and/or, L⁴-L⁵ are each independently selected from a single bond and substituted or unsubstituted C6-C30 arylenyl; optionally, L⁴-L⁵ are each independently selected from a single bond, phenylenyl and naphthylenyl, and further optionally, L⁴-L⁵ are each independently selected from a single bond;
and/or, the remaining of Ar¹-Ar³ are selected from hydrogen, deuterium, halogen, cyano, substituted or unsubstituted C6-C30 aryl, substituted or unsubstituted C3-C30 heteroaryl, substituted or unsubstituted dibenzofuryl, and substituted or unsubstituted dibenzothienyl.

13. The composition according to claim 8, wherein the structure of the compound N is as shown in any one of Formula 1-1 to Formula 1-17; 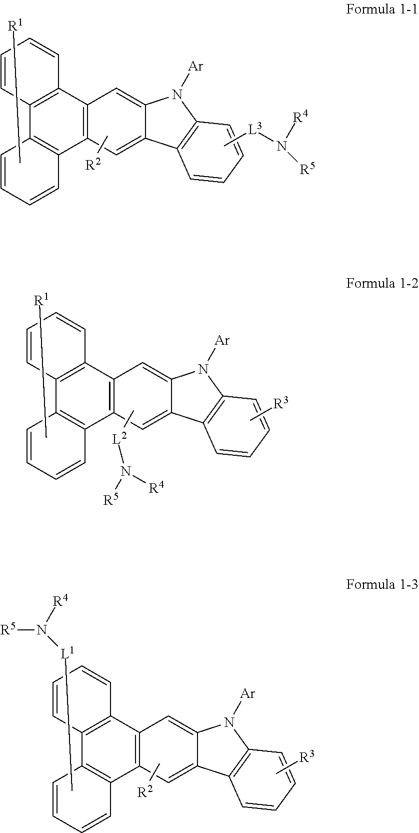 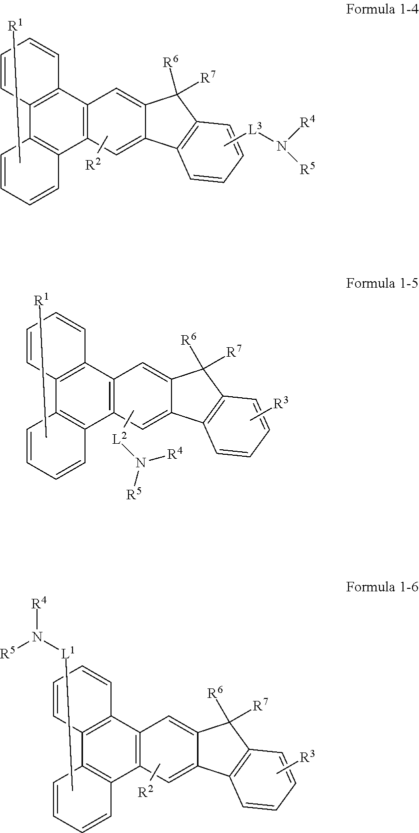 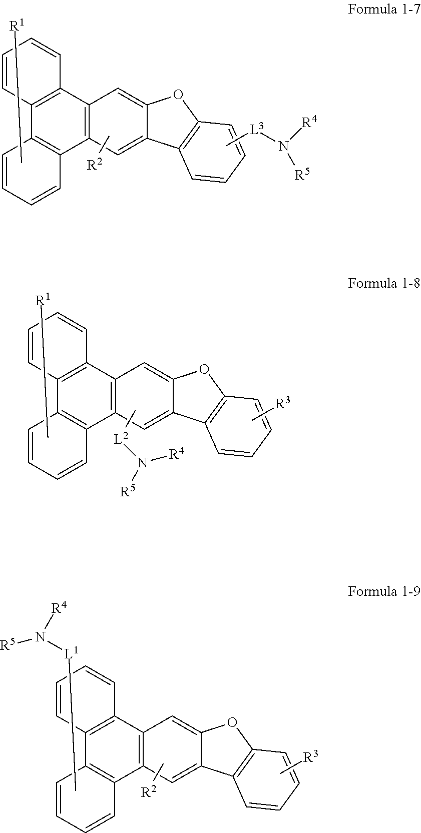 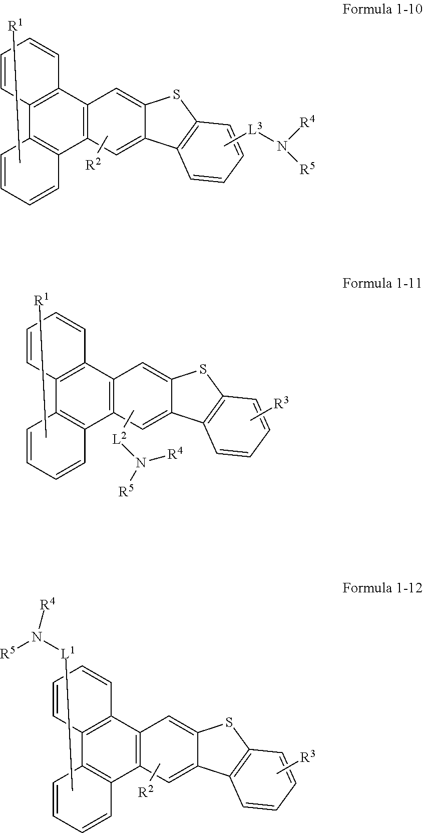 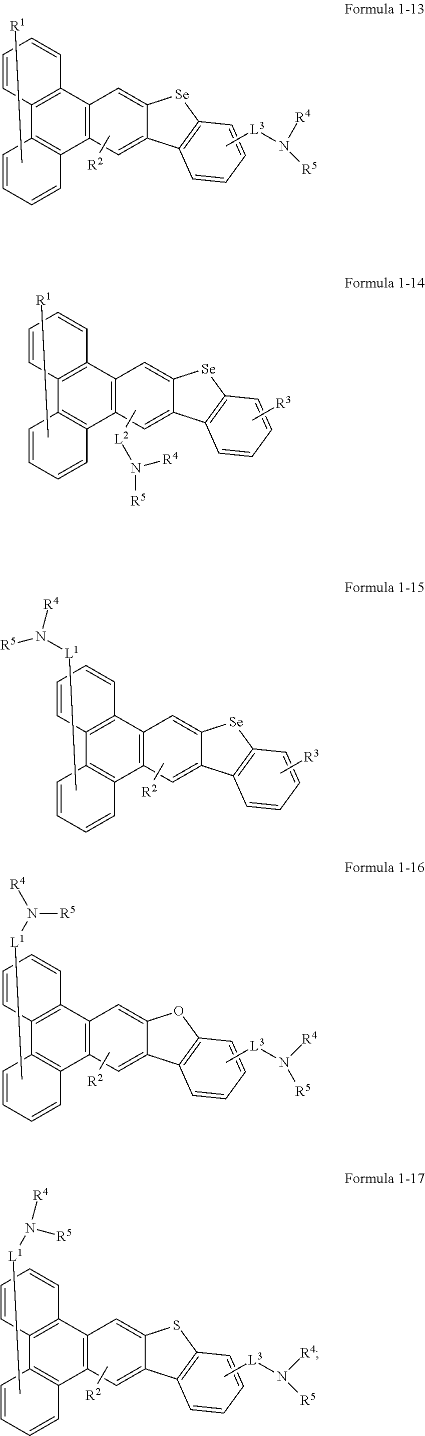
wherein R¹-R⁷, L¹-L³ and Ar are defined the same as that in claim 8.

14. The composition according to claim 8, wherein the structure of the compound N shown in Formula (1) is as shown in any one of N-1 to N-935:

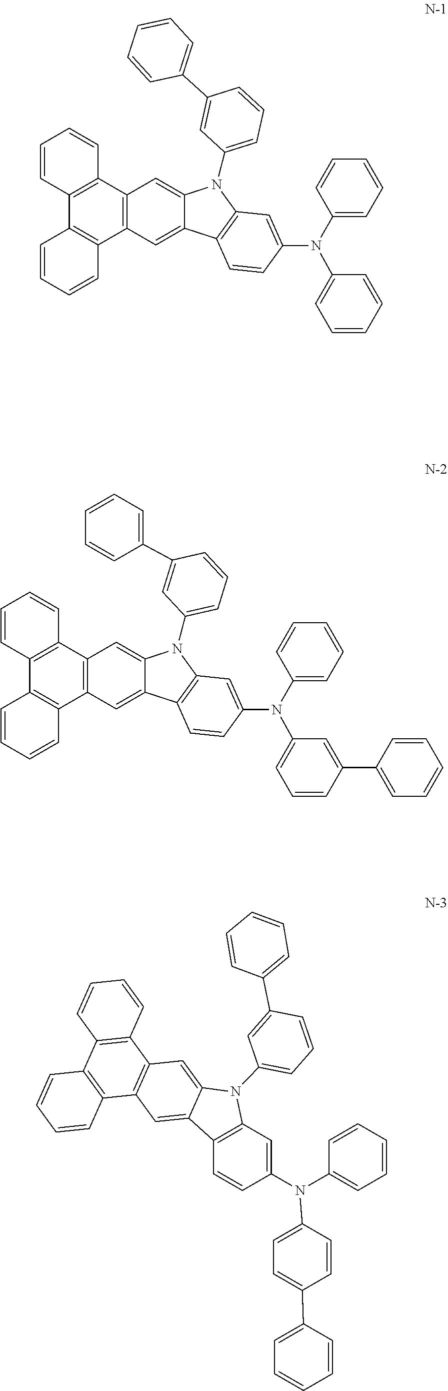 [SMILES: c1ccc(-c2cccc(-n3c4cc(N(c5ccccc5)c5ccccc5)ccc4c4cc5c6ccccc6c6ccccc6c5cc43)c2)cc1 ; c1ccc(-c2cccc(N(c3ccccc3)c3ccc4c5cc6c7ccccc7c7ccccc7c6cc5n(-c5cccc(-c6ccccc6)c5)c4c3)c2)cc1 ; c1ccc(-c2ccc(N(c3ccccc3)c3ccc4c5cc6c7ccccc7c7ccccc7c6cc5n(-c5cccc(-c6ccccc6)c5)c4c3)cc2)cc1]

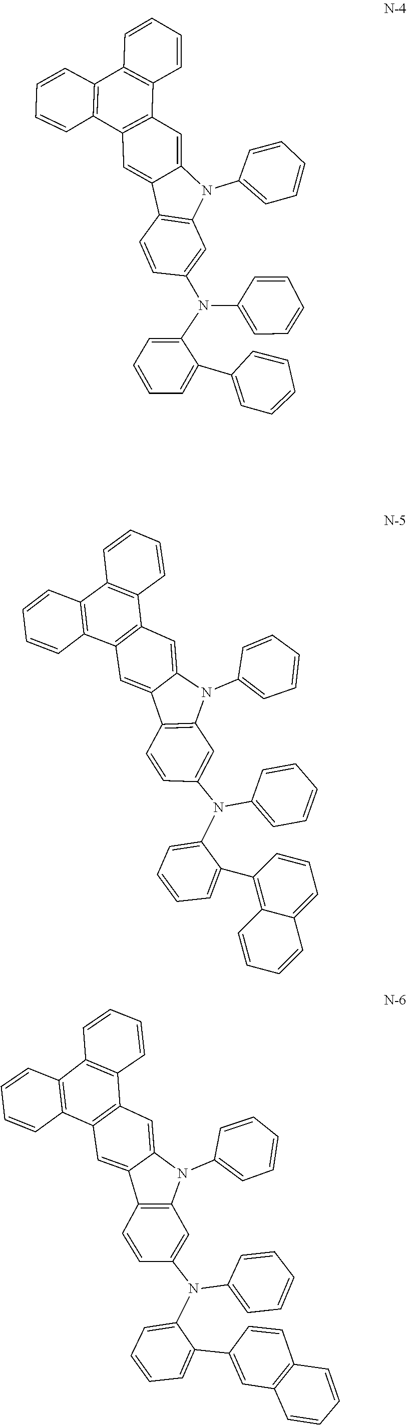 [SMILES: c1ccc(-c2ccccc2N(c2ccccc2)c2ccc3c4cc5c6ccccc6c6ccccc6c5cc4n(-c4ccccc4)c3c2)cc1 ; c1ccc(N(c2ccc3c4cc5c6ccccc6c6ccccc6c5cc4n(-c4ccccc4)c3c2)c2ccccc2-c2cccc3ccccc23)cc1 ; c1ccc(N(c2ccc3c4cc5c6ccccc6c6ccccc6c5cc4n(-c4ccccc4)c3c2)c2ccccc2-c2ccc3ccccc3c2)cc1]

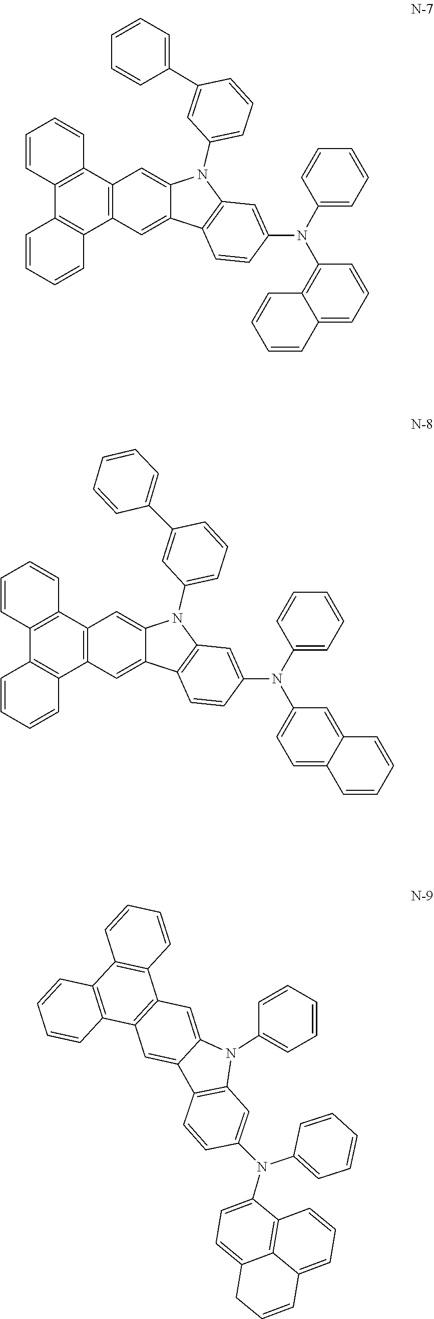 [SMILES: c1ccc(-c2cccc(-n3c4cc(N(c5ccccc5)c5cccc6ccccc56)ccc4c4cc5c6ccccc6c6ccccc6c5cc43)c2)cc1 ; c1ccc(-c2cccc(-n3c4cc(N(c5ccccc5)c5ccc6ccccc6c5)ccc4c4cc5c6ccccc6c6ccccc6c5cc43)c2)cc1 ; C1=Cc2cccc3c(N(c4ccccc4)c4ccc5c6cc7c8ccccc8c8ccccc8c7cc6n(-c6ccccc6)c5c4)ccc(c23)C1]

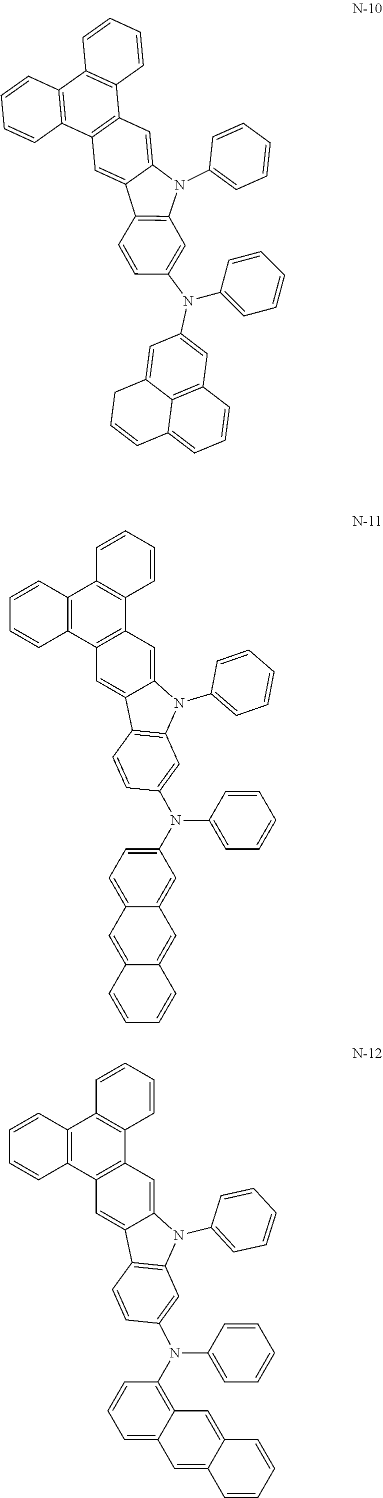

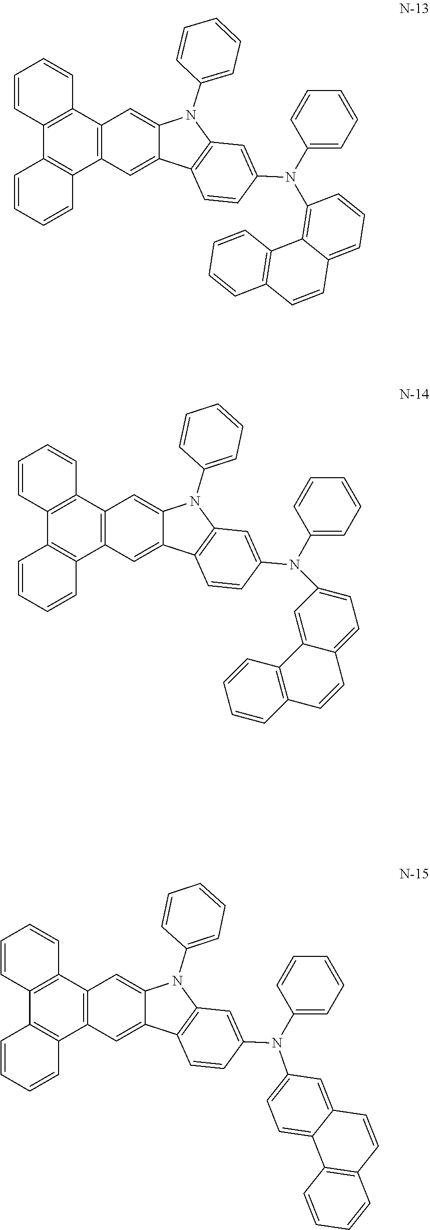 [SMILES: c1ccc(N(c2ccc3c4cc5c6ccccc6c6ccccc6c5cc4n(-c4ccccc4)c3c2)c2cccc3ccc4ccccc4c23)cc1 ; c1ccc(N(c2ccc3ccc4ccccc4c3c2)c2ccc3c4cc5c6ccccc6c6ccccc6c5cc4n(-c4ccccc4)c3c2)cc1 ; c1ccc(N(c2ccc3c(ccc4ccccc43)c2)c2ccc3c4cc5c6ccccc6c6ccccc6c5cc4n(-c4ccccc4)c3c2)cc1]

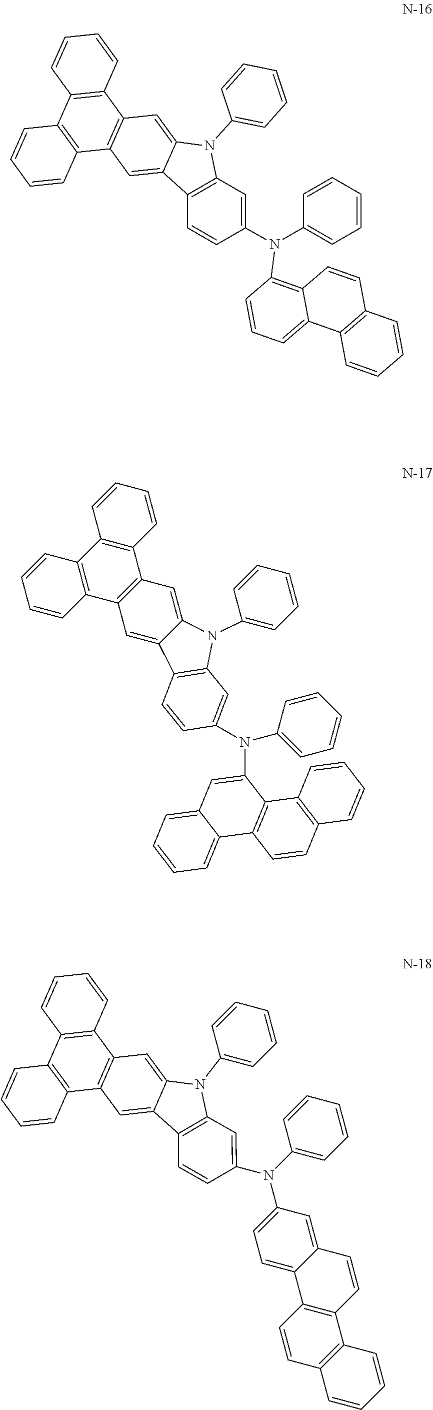 [SMILES: c1ccc(N(c2ccc3c4cc5c6ccccc6c6ccccc6c5cc4n(-c4ccccc4)c3c2)c2cccc3c2ccc2ccccc23)cc1 ; c1ccc(N(c2ccc3c4cc5c6ccccc6c6ccccc6c5cc4n(-c4ccccc4)c3c2)c2cc3ccccc3c3ccc4ccccc4c23)cc1 ; c1ccc(N(c2ccc3c(ccc4c5ccccc5ccc34)c2)c2ccc3c4cc5c6ccccc6c6ccccc6c5cc4n(-c4ccccc4)c3c2)cc1]

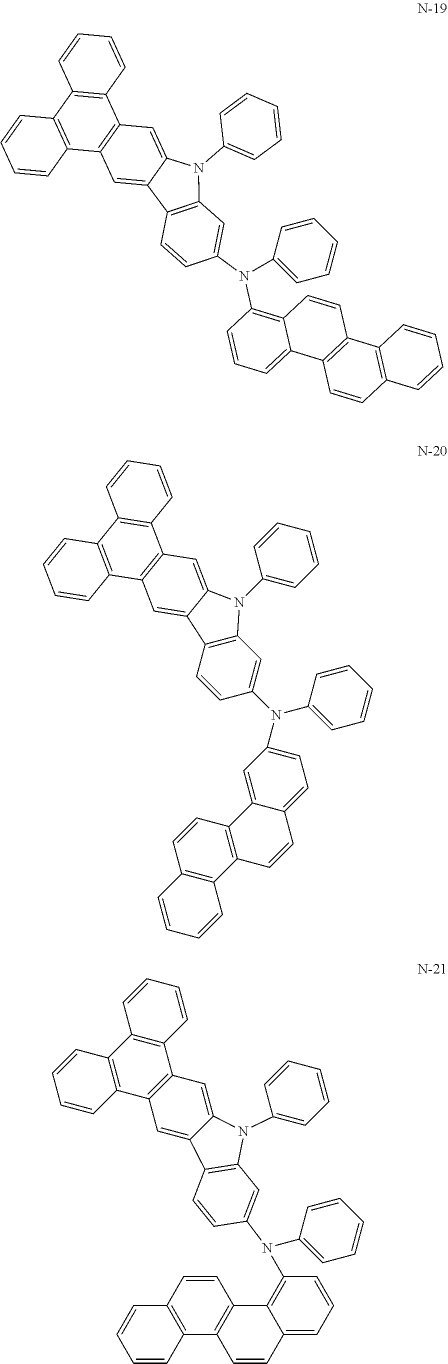 [SMILES: c1ccc(N(c2ccc3c4cc5c6ccccc6c6ccccc6c5cc4n(-c4ccccc4)c3c2)c2cccc3c2ccc2c4ccccc4ccc32)cc1 ; c1ccc(N(c2ccc3ccc4c5ccccc5ccc4c3c2)c2ccc3c4cc5c6ccccc6c6ccccc6c5cc4n(-c4ccccc4)c3c2)cc1 ; c1ccc(N(c2ccc3c4cc5c6ccccc6c6ccccc6c5cc4n(-c4ccccc4)c3c2)c2cccc3ccc4c5ccccc5ccc4c23)cc1]

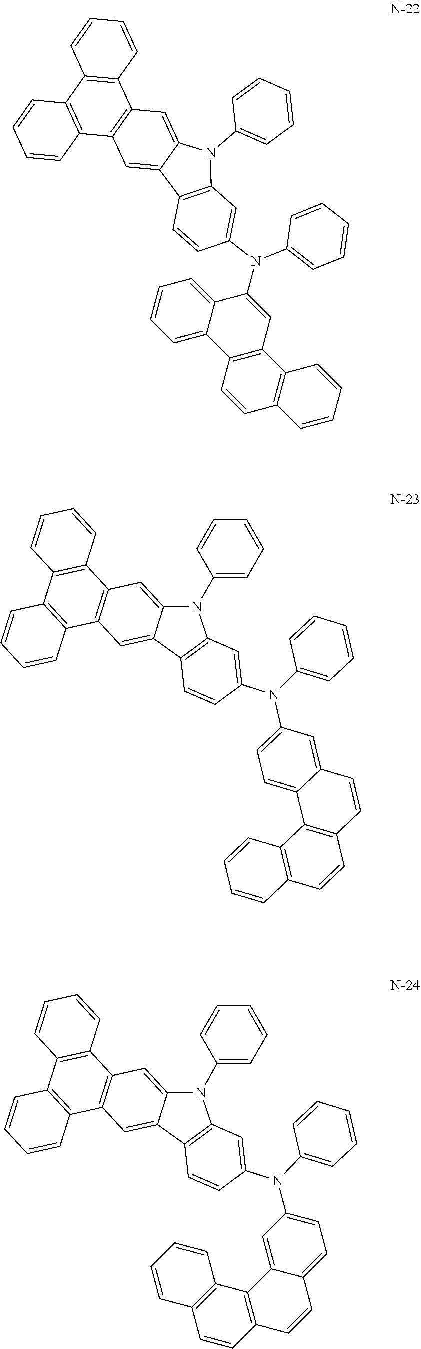 [SMILES: c1ccc(N(c2ccc3c4cc5c6ccccc6c6ccccc6c5cc4n(-c4ccccc4)c3c2)c2cc3c4ccccc4ccc3c3ccccc23)cc1 ; c1ccc(N(c2ccc3c(ccc4ccc5ccccc5c43)c2)c2ccc3c4cc5c6ccccc6c6ccccc6c5cc4n(-c4ccccc4)c3c2)cc1 ; c1ccc(N(c2ccc3ccc4ccc5ccccc5c4c3c2)c2ccc3c4cc5c6ccccc6c6ccccc6c5cc4n(-c4ccccc4)c3c2)cc1]

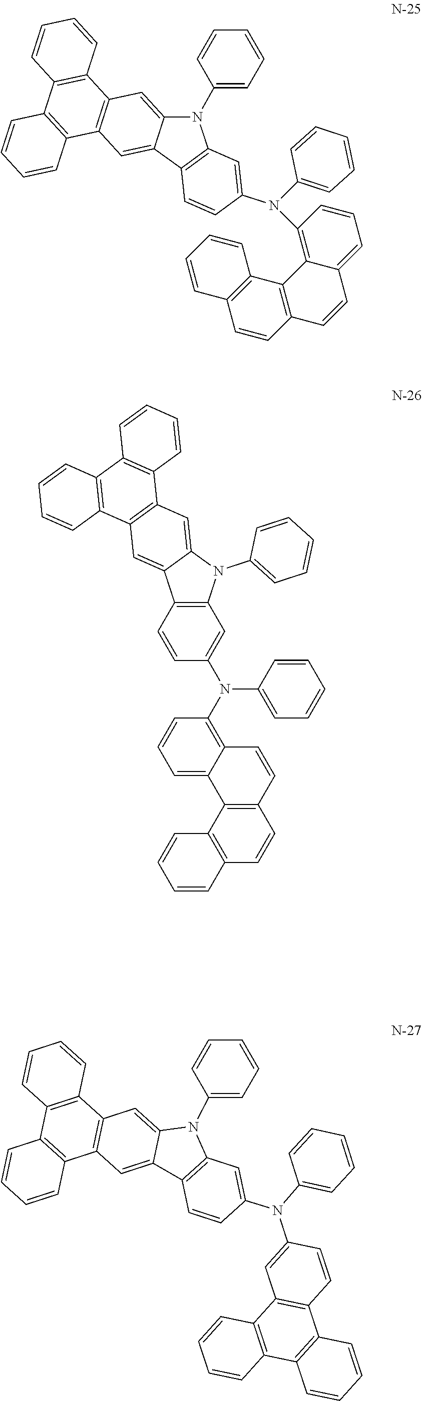 [SMILES: c1ccc(N(c2ccc3c4cc5c6ccccc6c6ccccc6c5cc4n(-c4ccccc4)c3c2)c2cccc3ccc4ccc5ccccc5c4c23)cc1 ; c1ccc(N(c2ccc3c4cc5c6ccccc6c6ccccc6c5cc4n(-c4ccccc4)c3c2)c2cccc3c2ccc2ccc4ccccc4c23)cc1 ; c1ccc(N(c2ccc3c4ccccc4c4ccccc4c3c2)c2ccc3c4cc5c6ccccc6c6ccccc6c5cc4n(-c4ccccc4)c3c2)cc1]

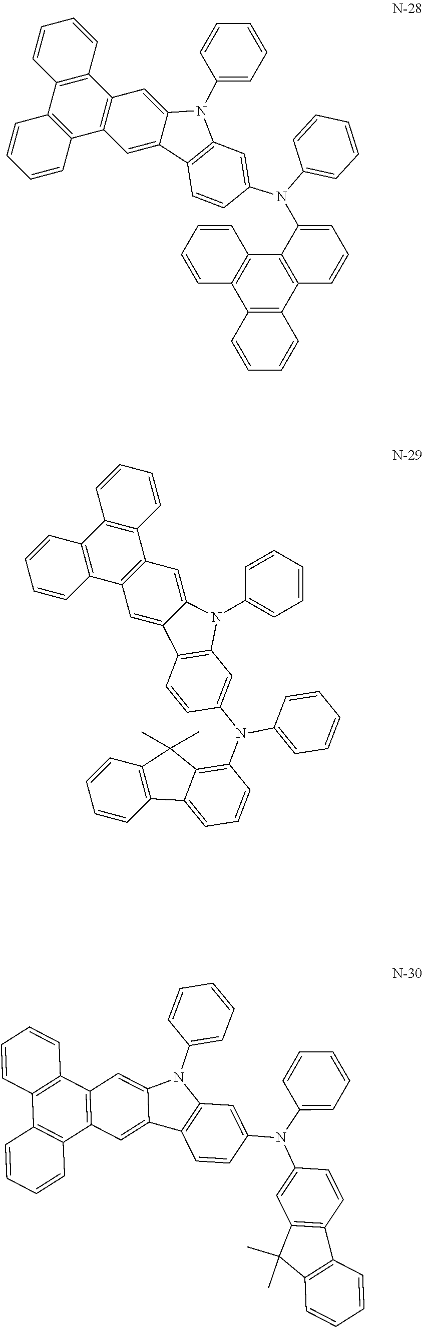 [SMILES: c1ccc(N(c2ccc3c4cc5c6ccccc6c6ccccc6c5cc4n(-c4ccccc4)c3c2)c2cccc3c4ccccc4c4ccccc4c23)cc1 ; CC1(C)c2ccccc2-c2cccc(N(c3ccccc3)c3ccc4c5cc6c7ccccc7c7ccccc7c6cc5n(-c5ccccc5)c4c3)c21 ; CC1(C)c2ccccc2-c2ccc(N(c3ccccc3)c3ccc4c5cc6c7ccccc7c7ccccc7c6cc5n(-c5ccccc5)c4c3)cc21]

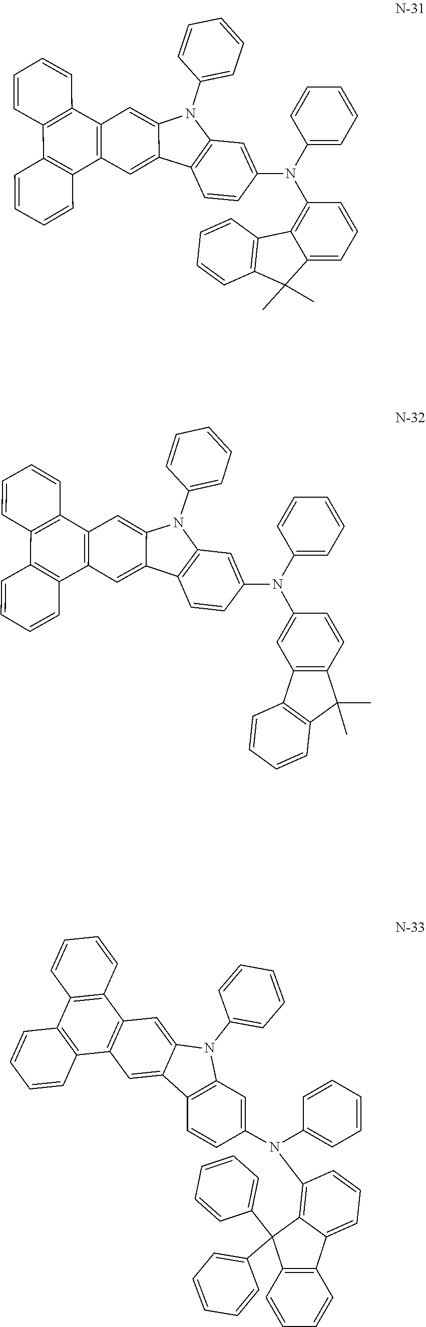 [SMILES: CC1(C)c2ccccc2-c2c(N(c3ccccc3)c3ccc4c5cc6c7ccccc7c7ccccc7c6cc5n(-c5ccccc5)c4c3)cccc21 ; CC1(C)c2ccccc2-c2cc(N(c3ccccc3)c3ccc4c5cc6c7ccccc7c7ccccc7c6cc5n(-c5ccccc5)c4c3)ccc21 ; c1ccc(N(c2ccc3c4cc5c6ccccc6c6ccccc6c5cc4n(-c4ccccc4)c3c2)c2cccc3c2C(c2ccccc2)(c2ccccc2)c2ccccc2-3)cc1]

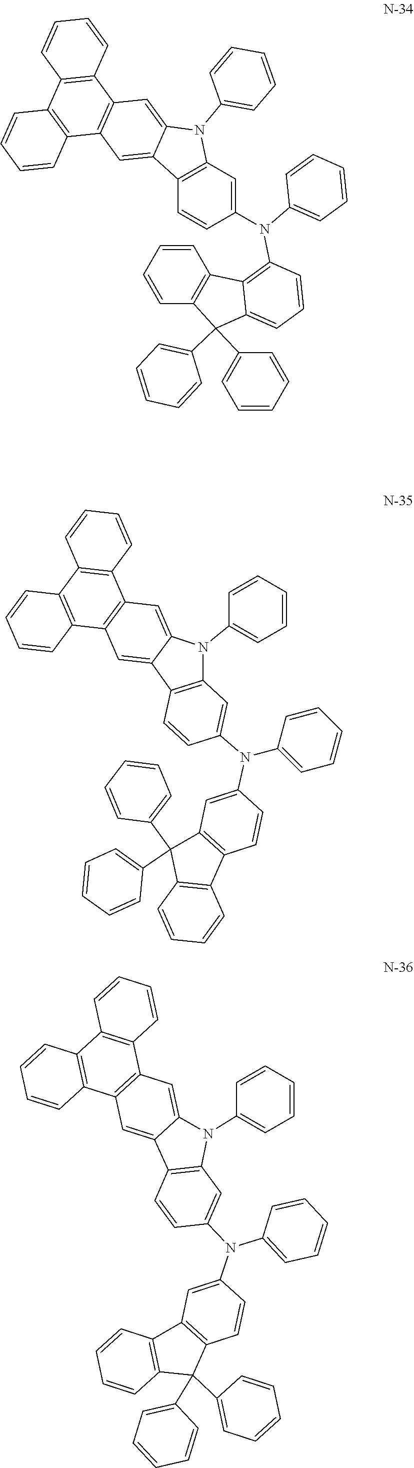 [SMILES: c1ccc(N(c2ccc3c4cc5c6ccccc6c6ccccc6c5cc4n(-c4ccccc4)c3c2)c2cccc3c2-c2ccccc2C3(c2ccccc2)c2ccccc2)cc1 ; c1ccc(N(c2ccc3c(c2)C(c2ccccc2)(c2ccccc2)c2ccccc2-3)c2ccc3c4cc5c6ccccc6c6ccccc6c5cc4n(-c4ccccc4)c3c2)cc1 ; c1ccc(N(c2ccc3c(c2)-c2ccccc2C3(c2ccccc2)c2ccccc2)c2ccc3c4cc5c6ccccc6c6ccccc6c5cc4n(-c4ccccc4)c3c2)cc1]

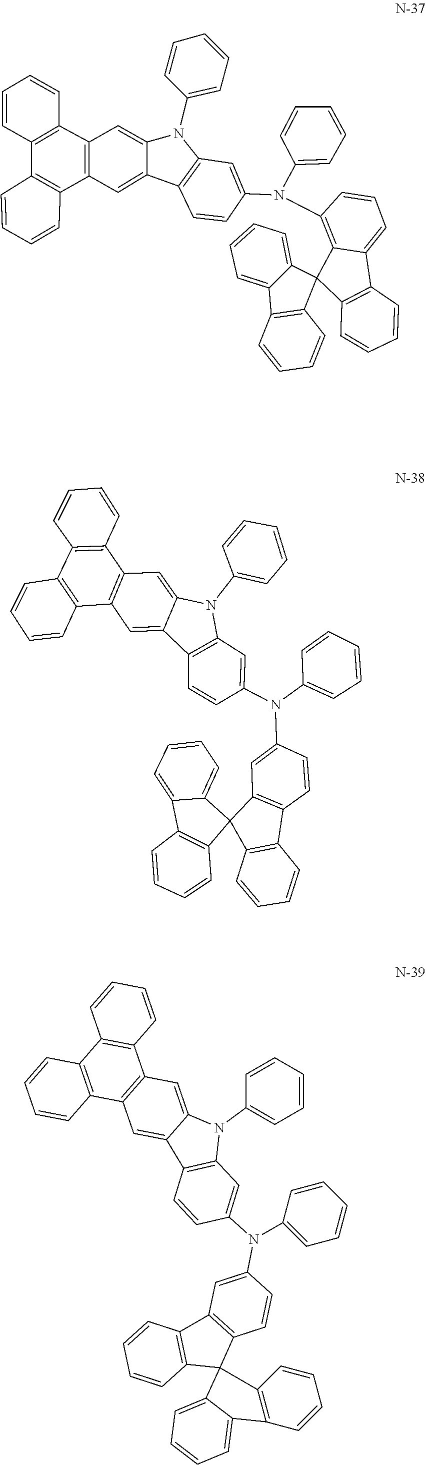 [SMILES: c1ccc(N(c2ccc3c4cc5c6ccccc6c6ccccc6c5cc4n(-c4ccccc4)c3c2)c2cccc3c2C2(c4ccccc4-c4ccccc42)c2ccccc2-3)cc1 ; c1ccc(N(c2ccc3c(c2)C2(c4ccccc4-c4ccccc42)c2ccccc2-3)c2ccc3c4cc5c6ccccc6c6ccccc6c5cc4n(-c4ccccc4)c3c2)cc1 ; c1ccc(N(c2ccc3c(c2)-c2ccccc2C32c3ccccc3-c3ccccc32)c2ccc3c4cc5c6ccccc6c6ccccc6c5cc4n(-c4ccccc4)c3c2)cc1]

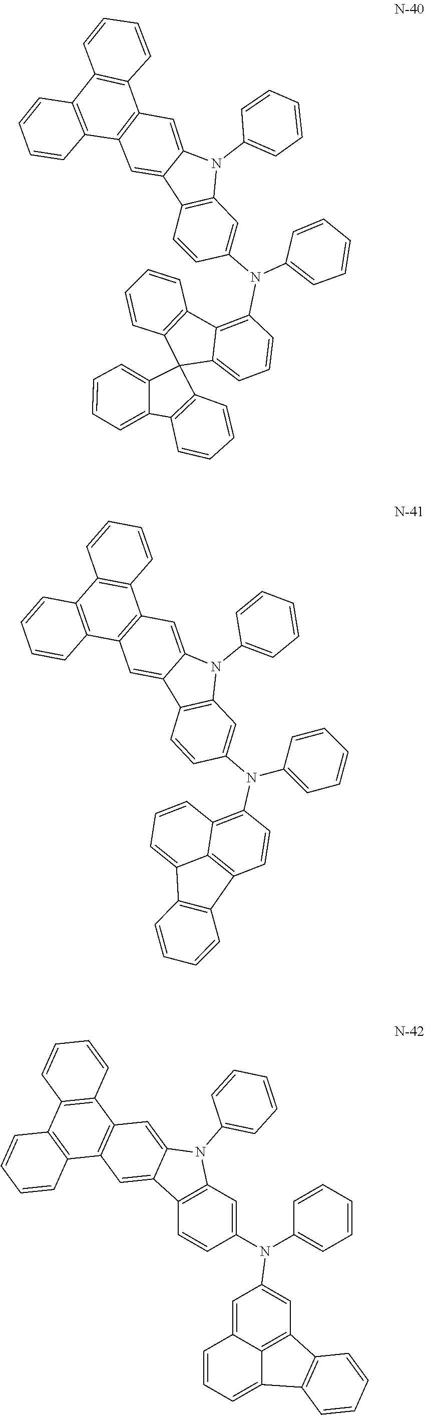 [SMILES: c1ccc(N(c2ccc3c4cc5c6ccccc6c6ccccc6c5cc4n(-c4ccccc4)c3c2)c2cccc3c2-c2ccccc2C32c3ccccc3-c3ccccc32)cc1 ; c1ccc(N(c2ccc3c4cc5c6ccccc6c6ccccc6c5cc4n(-c4ccccc4)c3c2)c2ccc3c4c(cccc24)-c2ccccc2-3)cc1 ; c1ccc(N(c2cc3c4c(cccc4c2)-c2ccccc2-3)c2ccc3c4cc5c6ccccc6c6ccccc6c5cc4n(-c4ccccc4)c3c2)cc1]

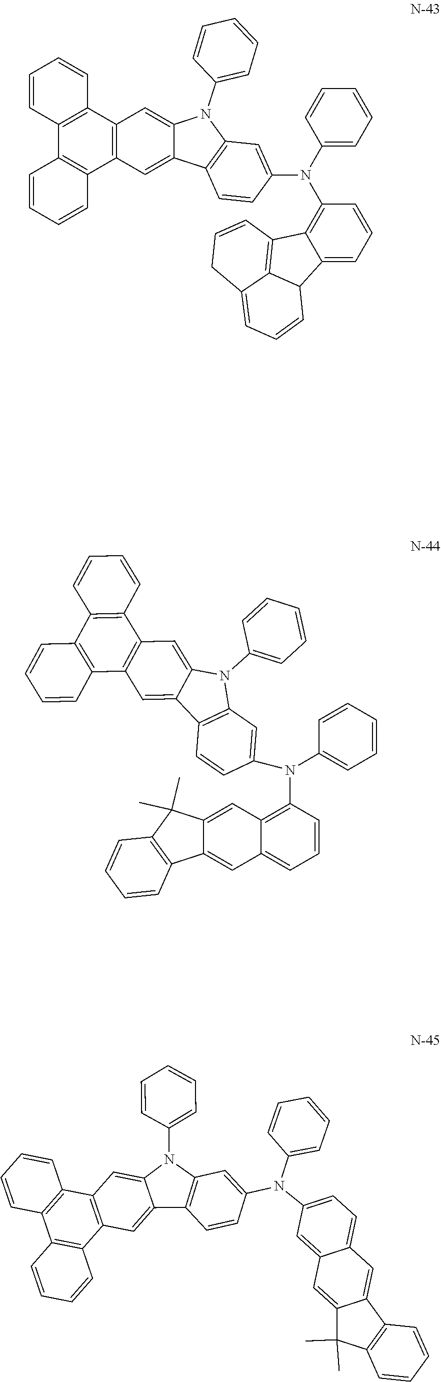 [SMILES: C1=CC2C3=C(C=CCC3=C1)c1c2cccc1N(c1ccccc1)c1ccc2c3cc4c5ccccc5c5ccccc5c4cc3n(-c3ccccc3)c2c1 ; CC1(C)c2ccccc2-c2cc3cccc(N(c4ccccc4)c4ccc5c6cc7c8ccccc8c8ccccc8c7cc6n(-c6ccccc6)c5c4)c3cc21 ; CC1(C)c2ccccc2-c2cc3ccc(N(c4ccccc4)c4ccc5c6cc7c8ccccc8c8ccccc8c7cc6n(-c6ccccc6)c5c4)cc3cc21]

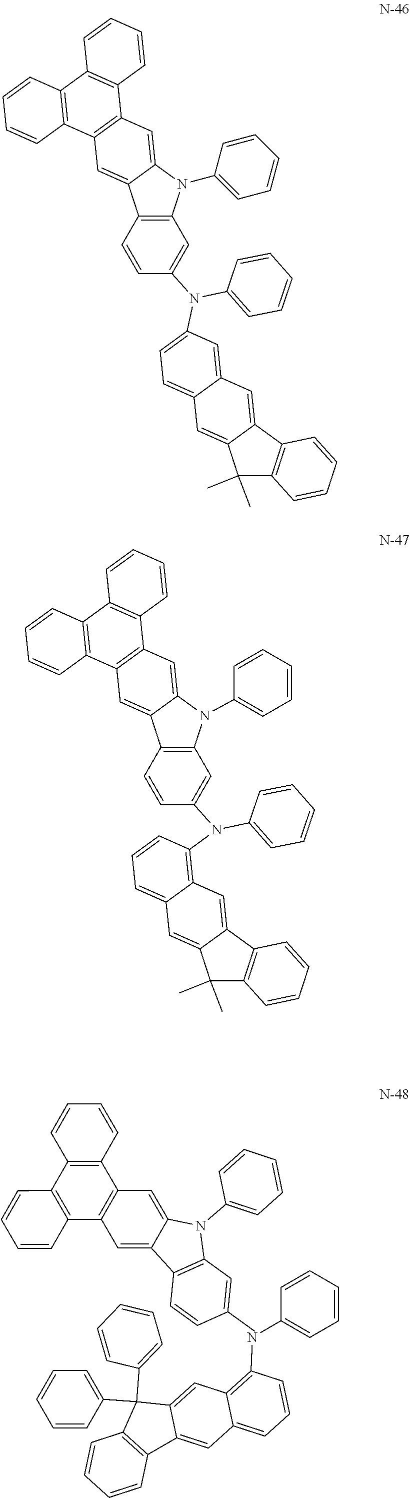

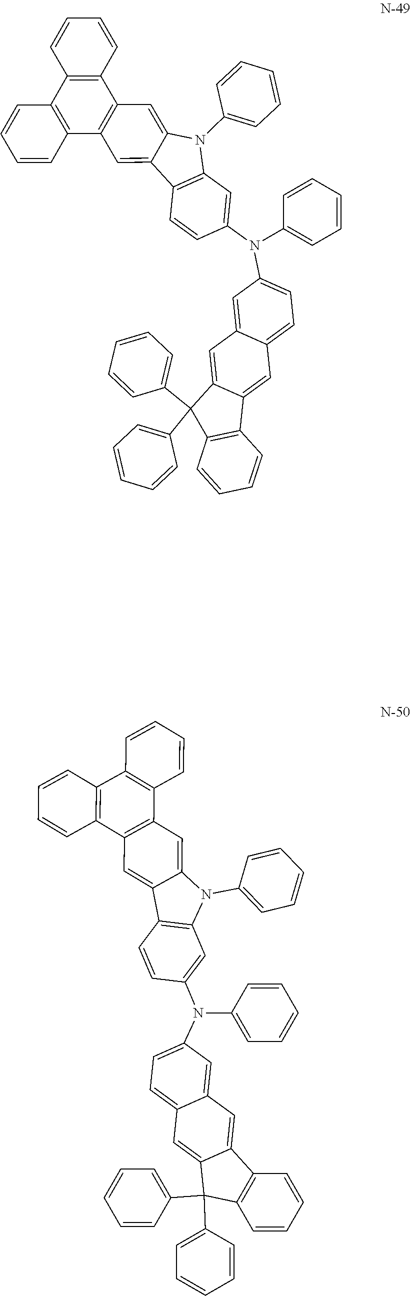 [SMILES: c1ccc(N(c2ccc3cc4c(cc3c2)C(c2ccccc2)(c2ccccc2)c2ccccc2-4)c2ccc3c4cc5c6ccccc6c6ccccc6c5cc4n(-c4ccccc4)c3c2)cc1 ; c1ccc(N(c2ccc3cc4c(cc3c2)-c2ccccc2C4(c2ccccc2)c2ccccc2)c2ccc3c4cc5c6ccccc6c6ccccc6c5cc4n(-c4ccccc4)c3c2)cc1]

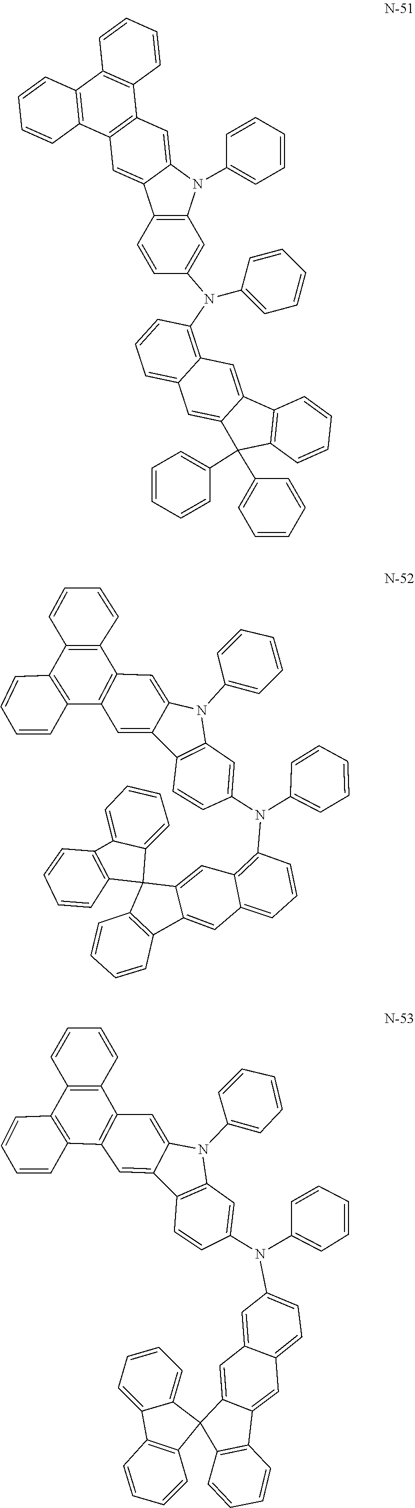 [SMILES: c1ccc(N(c2ccc3c4cc5c6ccccc6c6ccccc6c5cc4n(-c4ccccc4)c3c2)c2cccc3cc4c(cc23)-c2ccccc2C4(c2ccccc2)c2ccccc2)cc1 ; c1ccc(N(c2ccc3c4cc5c6ccccc6c6ccccc6c5cc4n(-c4ccccc4)c3c2)c2cccc3cc4c(cc23)C2(c3ccccc3-c3ccccc32)c2ccccc2-4)cc1 ; c1ccc(N(c2ccc3cc4c(cc3c2)C2(c3ccccc3-c3ccccc32)c2ccccc2-4)c2ccc3c4cc5c6ccccc6c6ccccc6c5cc4n(-c4ccccc4)c3c2)cc1]

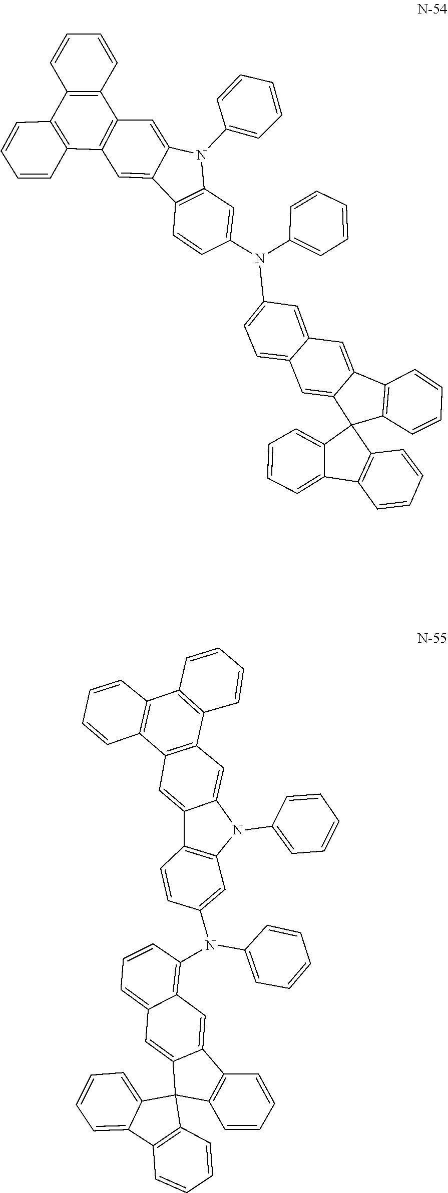 [SMILES: c1ccc(N(c2ccc3cc4c(cc3c2)-c2ccccc2C42c3ccccc3-c3ccccc32)c2ccc3c4cc5c6ccccc6c6ccccc6c5cc4n(-c4ccccc4)c3c2)cc1 ; c1ccc(N(c2ccc3c4cc5c6ccccc6c6ccccc6c5cc4n(-c4ccccc4)c3c2)c2cccc3cc4c(cc23)-c2ccccc2C42c3ccccc3-c3ccccc32)cc1]

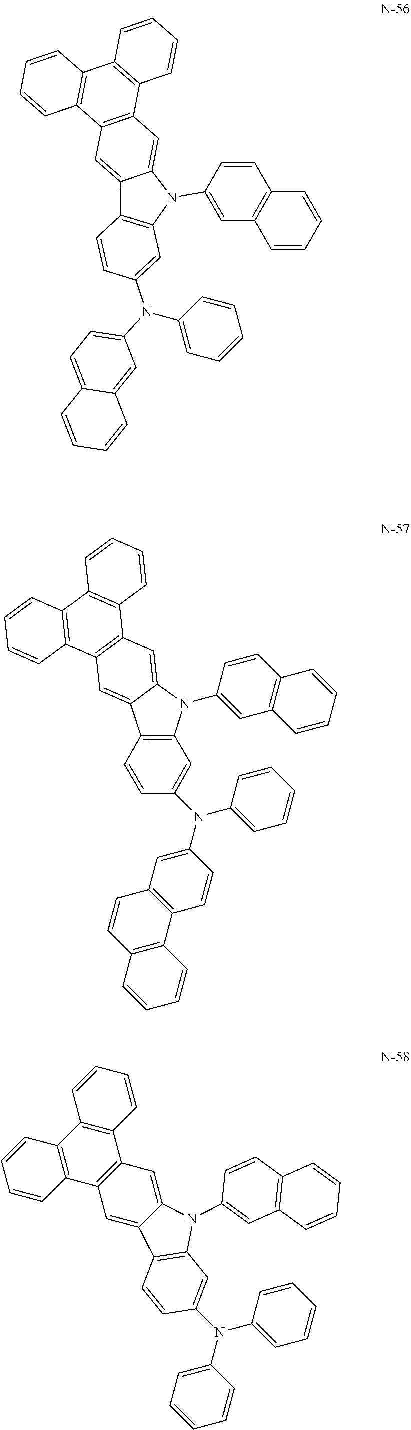 [SMILES: c1ccc(N(c2ccc3ccccc3c2)c2ccc3c4cc5c6ccccc6c6ccccc6c5cc4n(-c4ccc5ccccc5c4)c3c2)cc1 ; c1ccc(N(c2ccc3c(ccc4ccccc43)c2)c2ccc3c4cc5c6ccccc6c6ccccc6c5cc4n(-c4ccc5ccccc5c4)c3c2)cc1 ; c1ccc(N(c2ccccc2)c2ccc3c4cc5c6ccccc6c6ccccc6c5cc4n(-c4ccc5ccccc5c4)c3c2)cc1]

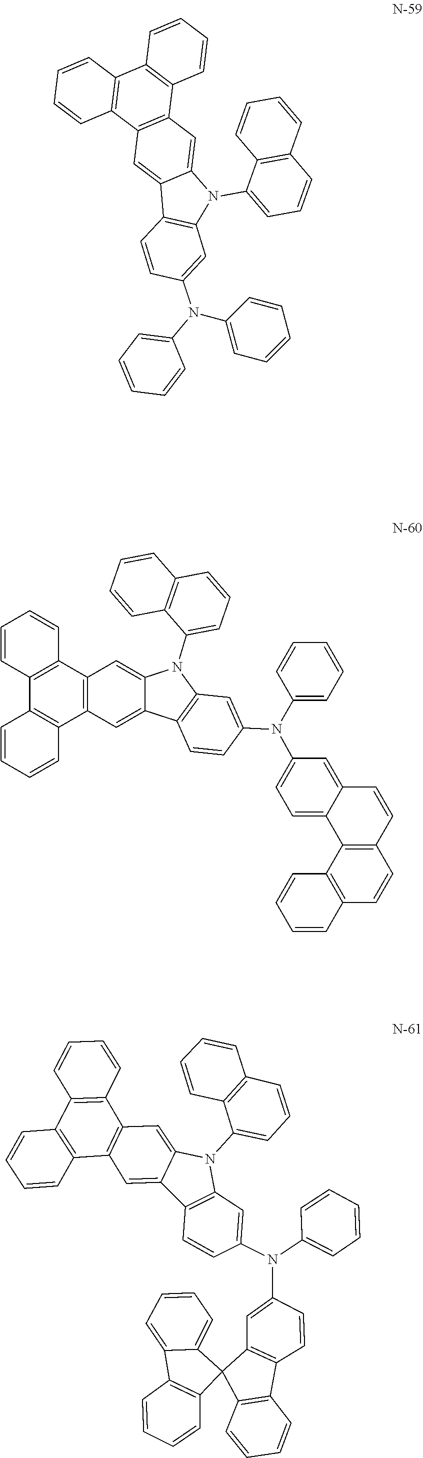 [SMILES: c1ccc(N(c2ccccc2)c2ccc3c4cc5c6ccccc6c6ccccc6c5cc4n(-c4cccc5ccccc45)c3c2)cc1 ; c1ccc(N(c2ccc3c(ccc4ccc5ccccc5c43)c2)c2ccc3c4cc5c6ccccc6c6ccccc6c5cc4n(-c4cccc5ccccc45)c3c2)cc1 ; c1ccc(N(c2ccc3c(c2)C2(c4ccccc4-c4ccccc42)c2ccccc2-3)c2ccc3c4cc5c6ccccc6c6ccccc6c5cc4n(-c4cccc5ccccc45)c3c2)cc1]

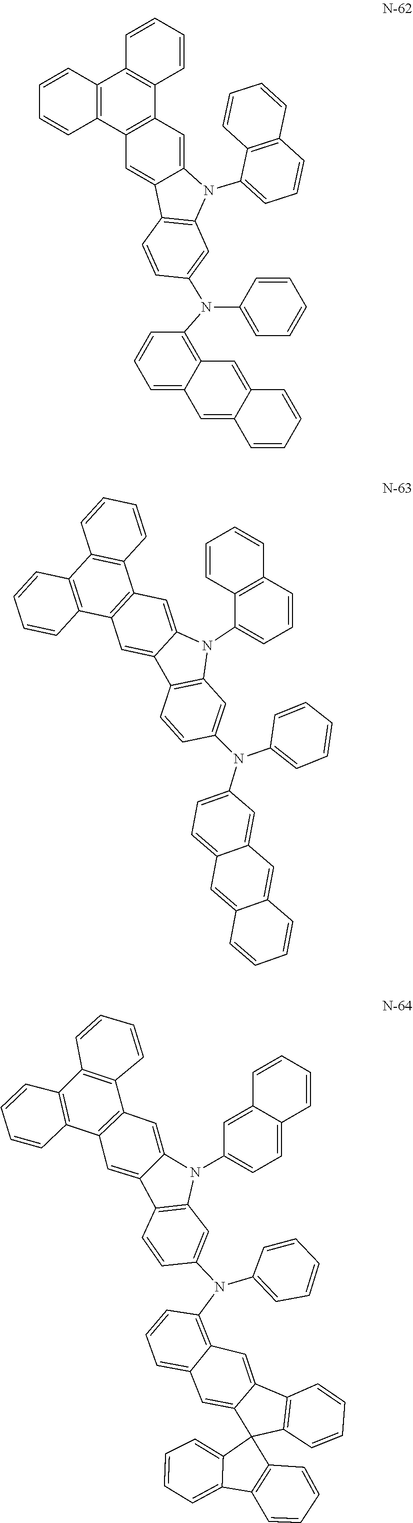 [SMILES: c1ccc(N(c2ccc3c4cc5c6ccccc6c6ccccc6c5cc4n(-c4cccc5ccccc45)c3c2)c2cccc3cc4ccccc4cc23)cc1 ; c1ccc(N(c2ccc3cc4ccccc4cc3c2)c2ccc3c4cc5c6ccccc6c6ccccc6c5cc4n(-c4cccc5ccccc45)c3c2)cc1 ; c1ccc(N(c2ccc3c4cc5c6ccccc6c6ccccc6c5cc4n(-c4ccc5ccccc5c4)c3c2)c2cccc3cc4c(cc23)-c2ccccc2C42c3ccccc3-c3ccccc32)cc1]

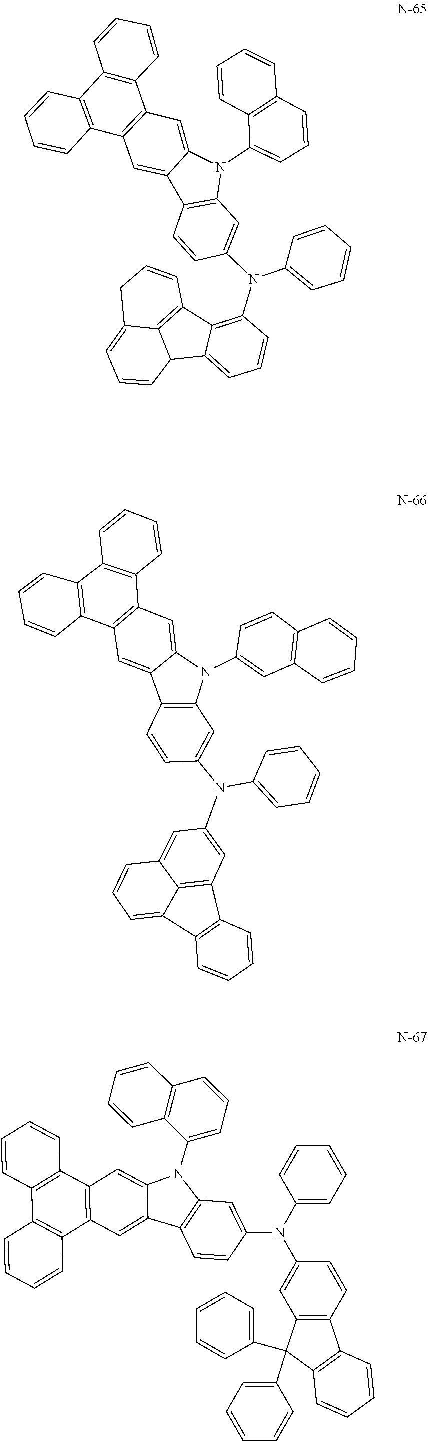 [SMILES: C1=CC2C3=C(C=CCC3=C1)c1c2cccc1N(c1ccccc1)c1ccc2c3cc4c5ccccc5c5ccccc5c4cc3n(-c3cccc4ccccc34)c2c1 ; c1ccc(N(c2cc3c4c(cccc4c2)-c2ccccc2-3)c2ccc3c4cc5c6ccccc6c6ccccc6c5cc4n(-c4ccc5ccccc5c4)c3c2)cc1 ; c1ccc(N(c2ccc3c(c2)C(c2ccccc2)(c2ccccc2)c2ccccc2-3)c2ccc3c4cc5c6ccccc6c6ccccc6c5cc4n(-c4cccc5ccccc45)c3c2)cc1]

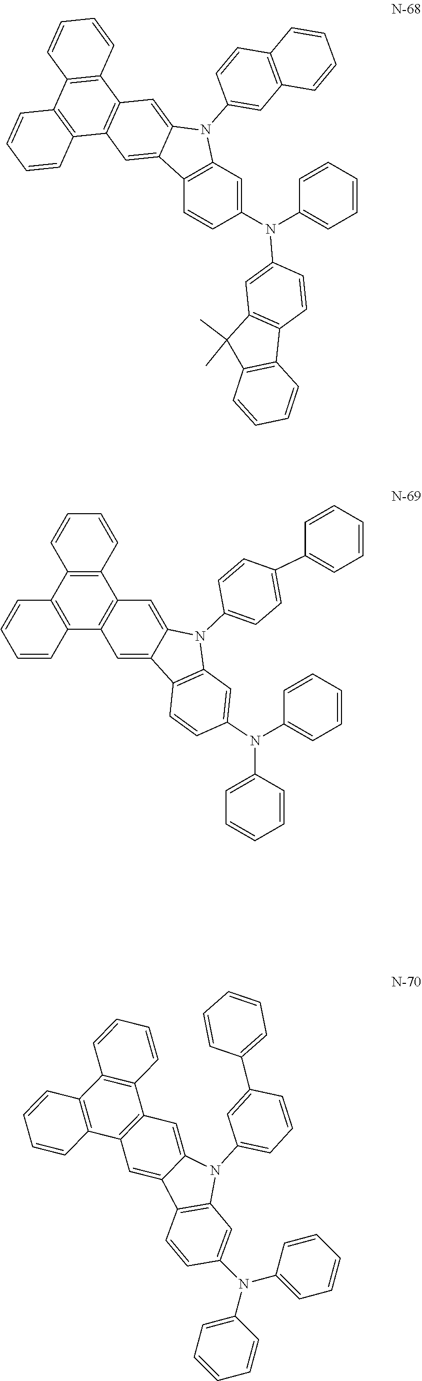 [SMILES: CC1(C)c2ccccc2-c2ccc(N(c3ccccc3)c3ccc4c5cc6c7ccccc7c7ccccc7c6cc5n(-c5ccc6ccccc6c5)c4c3)cc21 ; c1ccc(-c2ccc(-n3c4cc(N(c5ccccc5)c5ccccc5)ccc4c4cc5c6ccccc6c6ccccc6c5cc43)cc2)cc1 ; c1ccc(-c2cccc(-n3c4cc(N(c5ccccc5)c5ccccc5)ccc4c4cc5c6ccccc6c6ccccc6c5cc43)c2)cc1]

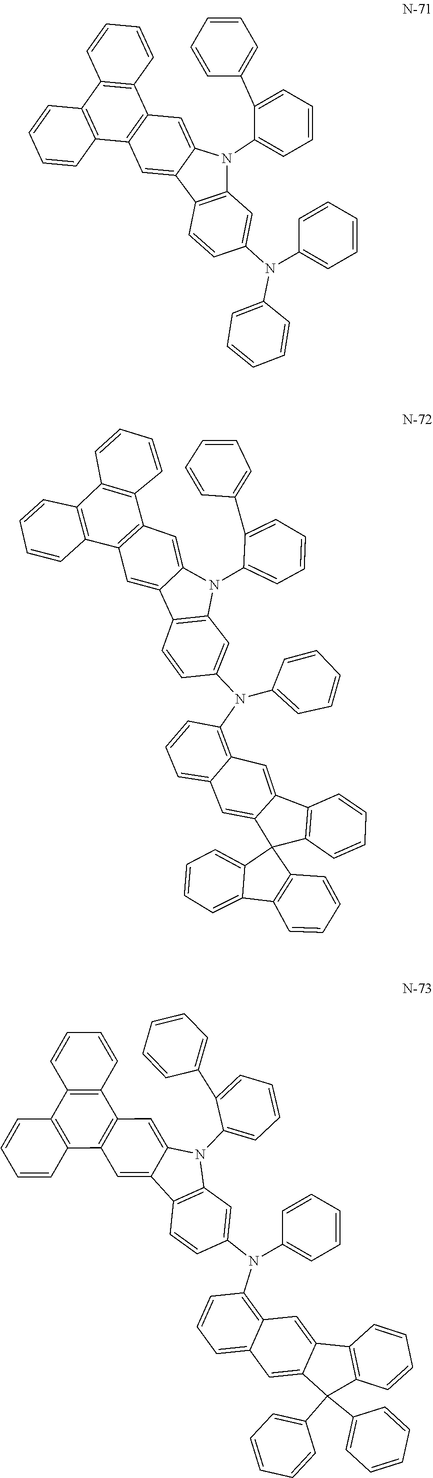 [SMILES: c1ccc(-c2ccccc2-n2c3cc(N(c4ccccc4)c4ccccc4)ccc3c3cc4c5ccccc5c5ccccc5c4cc32)cc1 ; c1ccc(-c2ccccc2-n2c3cc(N(c4ccccc4)c4cccc5cc6c(cc45)-c4ccccc4C64c5ccccc5-c5ccccc54)ccc3c3cc4c5ccccc5c5ccccc5c4cc32)cc1 ; c1ccc(-c2ccccc2-n2c3cc(N(c4ccccc4)c4cccc5cc6c(cc45)-c4ccccc4C6(c4ccccc4)c4ccccc4)ccc3c3cc4c5ccccc5c5ccccc5c4cc32)cc1]

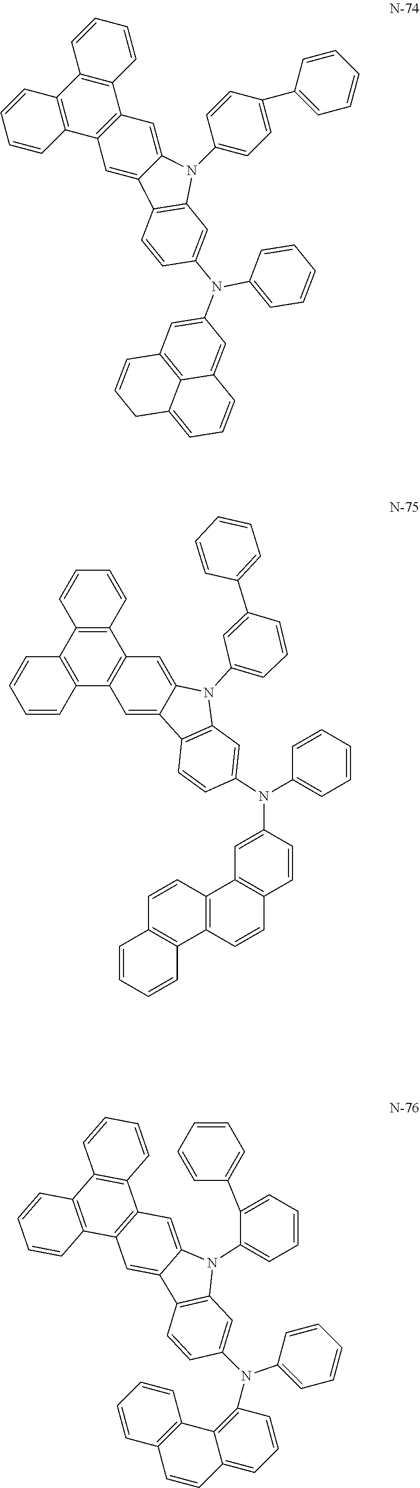 [SMILES: C1=Cc2cc(N(c3ccccc3)c3ccc4c5cc6c7ccccc7c7ccccc7c6cc5n(-c5ccc(-c6ccccc6)cc5)c4c3)cc3cccc(c23)C1 ; c1ccc(-c2cccc(-n3c4cc(N(c5ccccc5)c5ccc6ccc7c8ccccc8ccc7c6c5)ccc4c4cc5c6ccccc6c6ccccc6c5cc43)c2)cc1 ; c1ccc(-c2ccccc2-n2c3cc(N(c4ccccc4)c4cccc5ccc6ccccc6c45)ccc3c3cc4c5ccccc5c5ccccc5c4cc32)cc1]

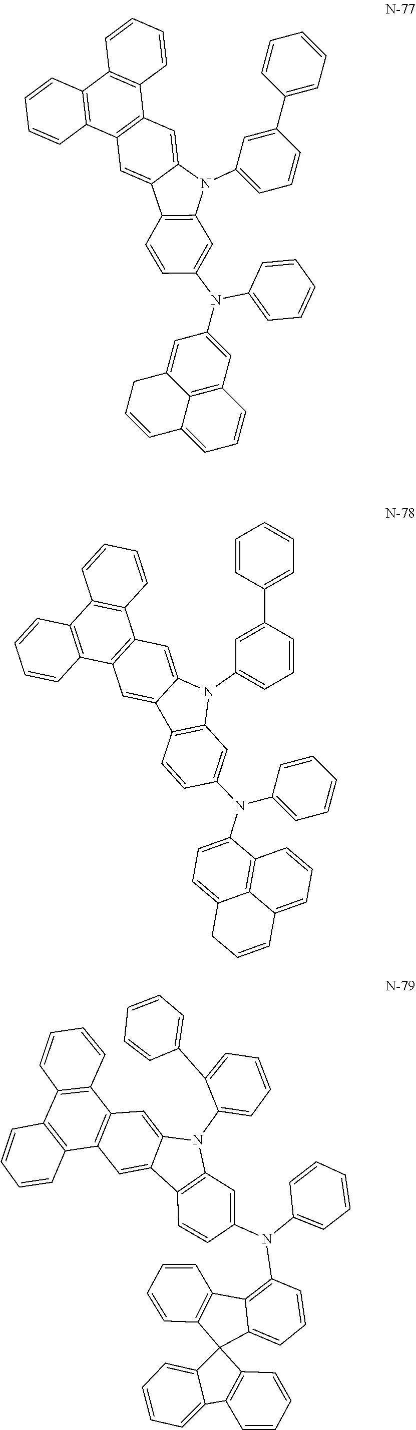 [SMILES: C1=Cc2cccc3cc(N(c4ccccc4)c4ccc5c6cc7c8ccccc8c8ccccc8c7cc6n(-c6cccc(-c7ccccc7)c6)c5c4)cc(c23)C1 ; C1=Cc2cccc3c(N(c4ccccc4)c4ccc5c6cc7c8ccccc8c8ccccc8c7cc6n(-c6cccc(-c7ccccc7)c6)c5c4)ccc(c23)C1 ; c1ccc(-c2ccccc2-n2c3cc(N(c4ccccc4)c4cccc5c4-c4ccccc4C54c5ccccc5-c5ccccc54)ccc3c3cc4c5ccccc5c5ccccc5c4cc32)cc1]

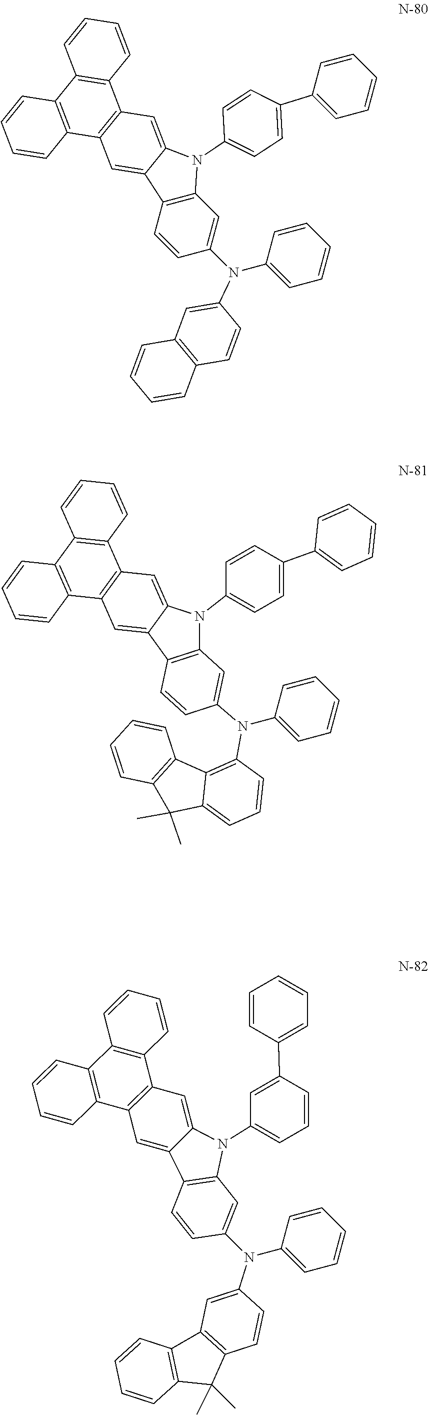 [SMILES: c1ccc(-c2ccc(-n3c4cc(N(c5ccccc5)c5ccc6ccccc6c5)ccc4c4cc5c6ccccc6c6ccccc6c5cc43)cc2)cc1 ; CC1(C)c2ccccc2-c2c(N(c3ccccc3)c3ccc4c5cc6c7ccccc7c7ccccc7c6cc5n(-c5ccc(-c6ccccc6)cc5)c4c3)cccc21 ; CC1(C)c2ccccc2-c2cc(N(c3ccccc3)c3ccc4c5cc6c7ccccc7c7ccccc7c6cc5n(-c5cccc(-c6ccccc6)c5)c4c3)ccc21]

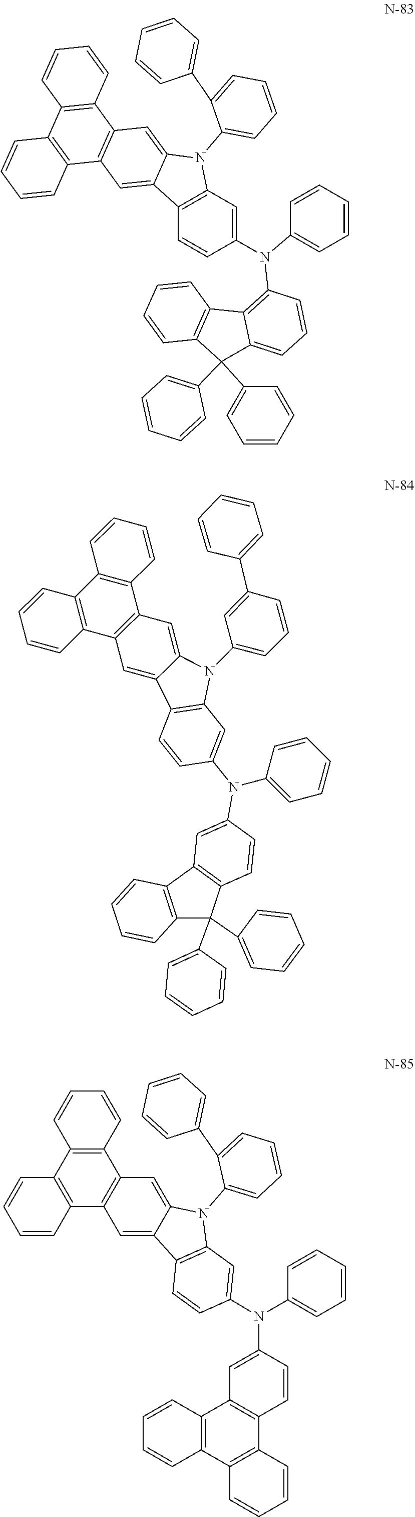 [SMILES: c1ccc(-c2ccccc2-n2c3cc(N(c4ccccc4)c4cccc5c4-c4ccccc4C5(c4ccccc4)c4ccccc4)ccc3c3cc4c5ccccc5c5ccccc5c4cc32)cc1 ; c1ccc(-c2cccc(-n3c4cc(N(c5ccccc5)c5ccc6c(c5)-c5ccccc5C6(c5ccccc5)c5ccccc5)ccc4c4cc5c6ccccc6c6ccccc6c5cc43)c2)cc1 ; c1ccc(-c2ccccc2-n2c3cc(N(c4ccccc4)c4ccc5c6ccccc6c6ccccc6c5c4)ccc3c3cc4c5ccccc5c5ccccc5c4cc32)cc1]

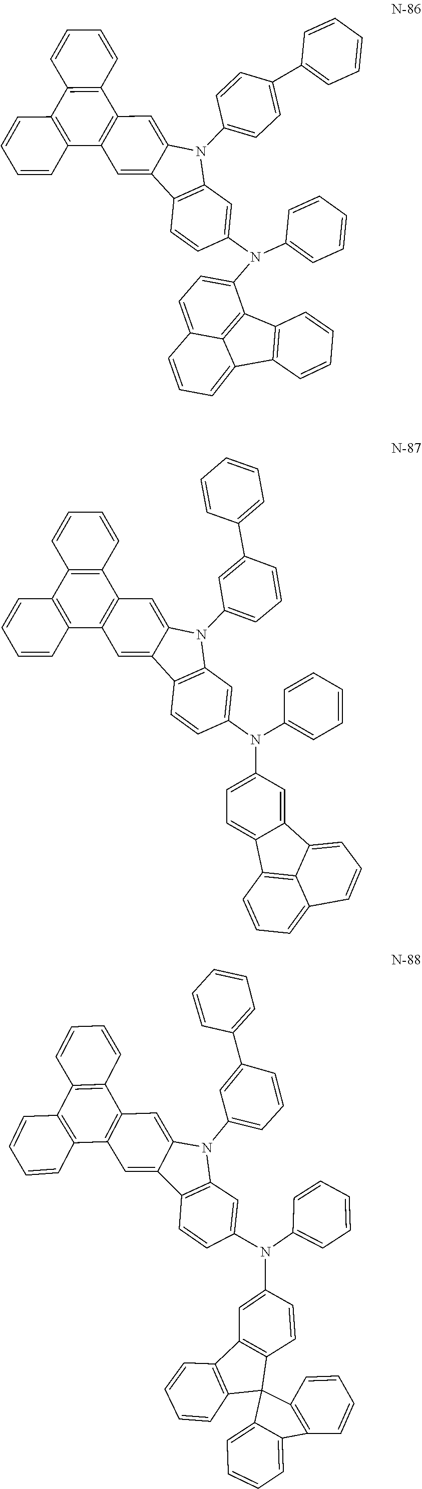 [SMILES: c1ccc(-c2ccc(-n3c4cc(N(c5ccccc5)c5ccc6cccc7c6c5-c5ccccc5-7)ccc4c4cc5c6ccccc6c6ccccc6c5cc43)cc2)cc1 ; c1ccc(-c2cccc(-n3c4cc(N(c5ccccc5)c5ccc6c(c5)-c5cccc7cccc-6c57)ccc4c4cc5c6ccccc6c6ccccc6c5cc43)c2)cc1 ; c1ccc(-c2cccc(-n3c4cc(N(c5ccccc5)c5ccc6c(c5)-c5ccccc5C65c6ccccc6-c6ccccc65)ccc4c4cc5c6ccccc6c6ccccc6c5cc43)c2)cc1]

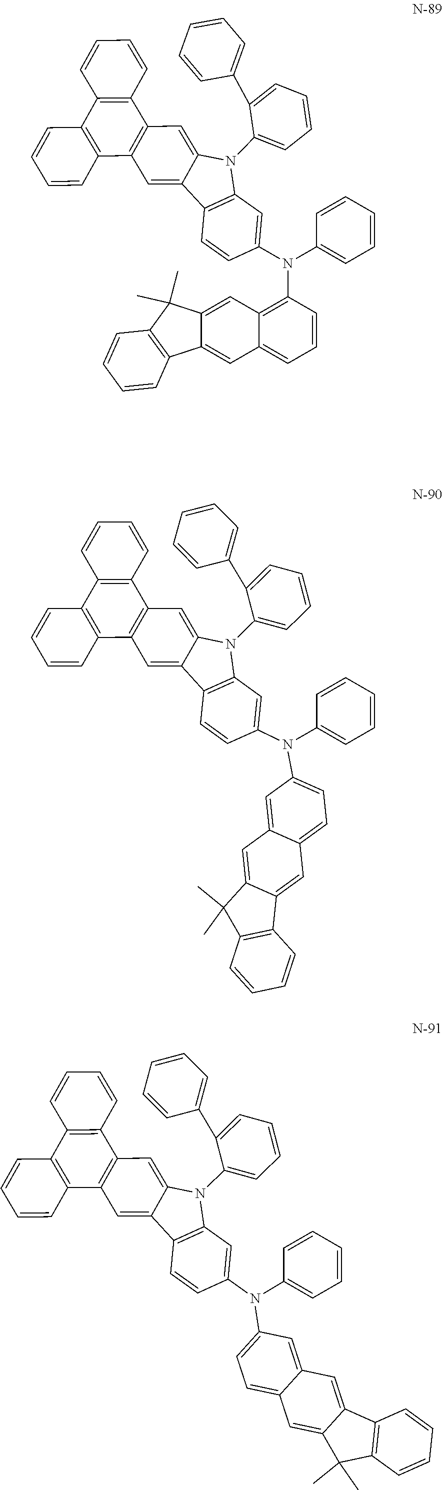 [SMILES: CC1(C)c2ccccc2-c2cc3cccc(N(c4ccccc4)c4ccc5c6cc7c8ccccc8c8ccccc8c7cc6n(-c6ccccc6-c6ccccc6)c5c4)c3cc21 ; CC1(C)c2ccccc2-c2cc3ccc(N(c4ccccc4)c4ccc5c6cc7c8ccccc8c8ccccc8c7cc6n(-c6ccccc6-c6ccccc6)c5c4)cc3cc21 ; CC1(C)c2ccccc2-c2cc3cc(N(c4ccccc4)c4ccc5c6cc7c8ccccc8c8ccccc8c7cc6n(-c6ccccc6-c6ccccc6)c5c4)ccc3cc21]

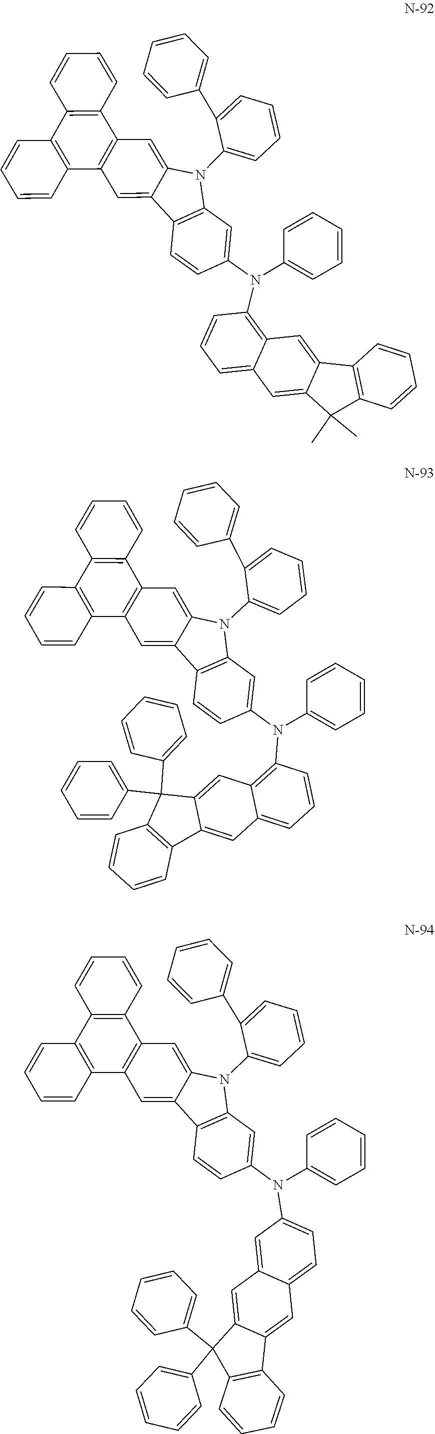 [SMILES: CC1(C)c2ccccc2-c2cc3c(N(c4ccccc4)c4ccc5c6cc7c8ccccc8c8ccccc8c7cc6n(-c6ccccc6-c6ccccc6)c5c4)cccc3cc21 ; c1ccc(-c2ccccc2-n2c3cc(N(c4ccccc4)c4cccc5cc6c(cc45)C(c4ccccc4)(c4ccccc4)c4ccccc4-6)ccc3c3cc4c5ccccc5c5ccccc5c4cc32)cc1 ; c1ccc(-c2ccccc2-n2c3cc(N(c4ccccc4)c4ccc5cc6c(cc5c4)C(c4ccccc4)(c4ccccc4)c4ccccc4-6)ccc3c3cc4c5ccccc5c5ccccc5c4cc32)cc1]

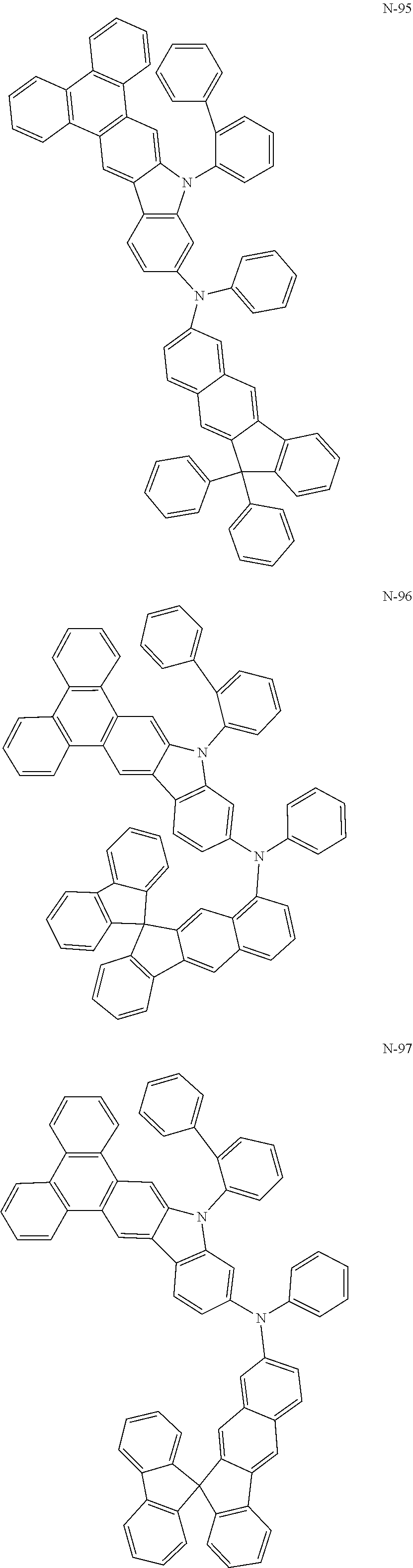 [SMILES: c1ccc(-c2ccccc2-n2c3cc(N(c4ccccc4)c4ccc5cc6c(cc5c4)-c4ccccc4C6(c4ccccc4)c4ccccc4)ccc3c3cc4c5ccccc5c5ccccc5c4cc32)cc1 ; c1ccc(-c2ccccc2-n2c3cc(N(c4ccccc4)c4cccc5cc6c(cc45)C4(c5ccccc5-c5ccccc54)c4ccccc4-6)ccc3c3cc4c5ccccc5c5ccccc5c4cc32)cc1 ; c1ccc(-c2ccccc2-n2c3cc(N(c4ccccc4)c4ccc5cc6c(cc5c4)C4(c5ccccc5-c5ccccc54)c4ccccc4-6)ccc3c3cc4c5ccccc5c5ccccc5c4cc32)cc1]

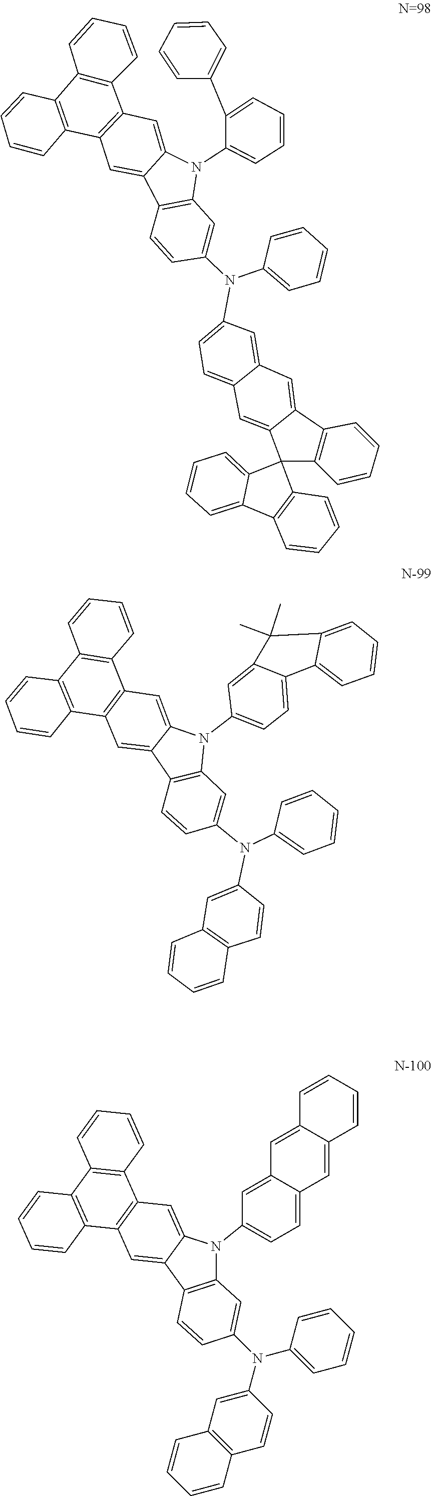 [SMILES: c1ccc(-c2ccccc2-n2c3cc(N(c4ccccc4)c4ccc5cc6c(cc5c4)-c4ccccc4C64c5ccccc5-c5ccccc54)ccc3c3cc4c5ccccc5c5ccccc5c4cc32)cc1 ; CC1(C)c2ccccc2-c2ccc(-n3c4cc(N(c5ccccc5)c5ccc6ccccc6c5)ccc4c4cc5c6ccccc6c6ccccc6c5cc43)cc21 ; c1ccc(N(c2ccc3ccccc3c2)c2ccc3c4cc5c6ccccc6c6ccccc6c5cc4n(-c4ccc5cc6ccccc6cc5c4)c3c2)cc1]

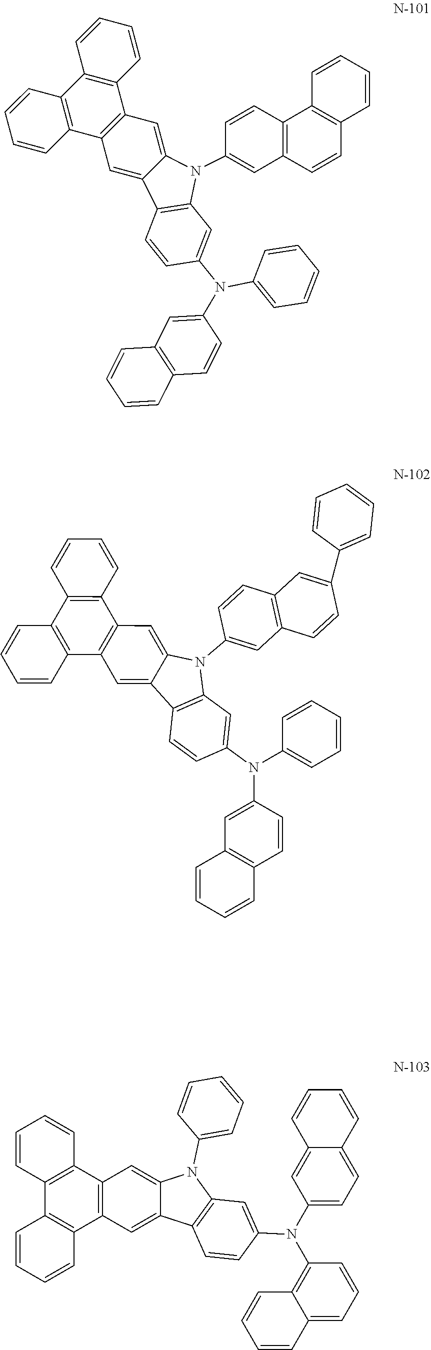 [SMILES: c1ccc(N(c2ccc3ccccc3c2)c2ccc3c4cc5c6ccccc6c6ccccc6c5cc4n(-c4ccc5c(ccc6ccccc65)c4)c3c2)cc1 ; c1ccc(-c2ccc3cc(-n4c5cc(N(c6ccccc6)c6ccc7ccccc7c6)ccc5c5cc6c7ccccc7c7ccccc7c6cc54)ccc3c2)cc1 ; c1ccc(-n2c3cc(N(c4ccc5ccccc5c4)c4cccc5ccccc45)ccc3c3cc4c5ccccc5c5ccccc5c4cc32)cc1]

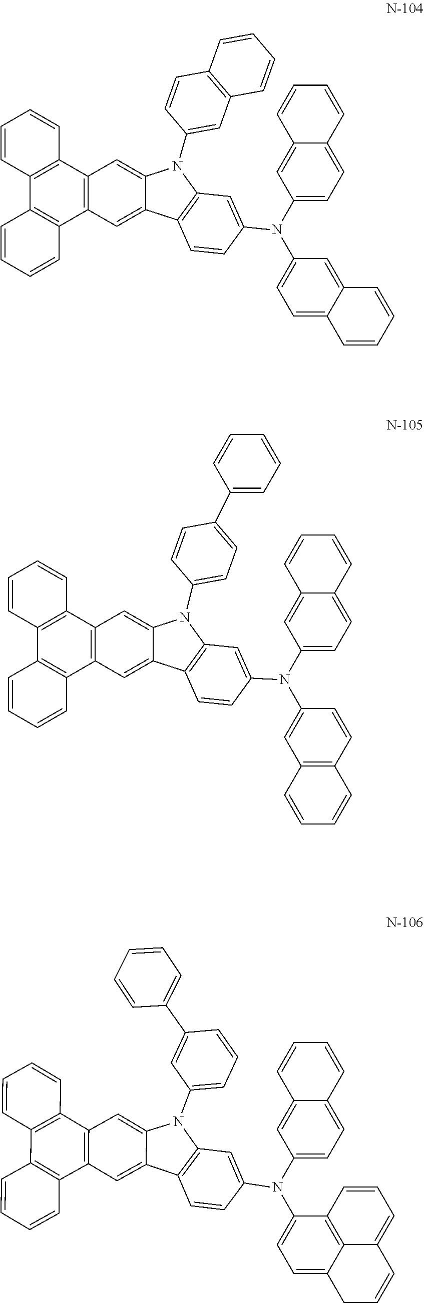 [SMILES: c1ccc2cc(N(c3ccc4ccccc4c3)c3ccc4c5cc6c7ccccc7c7ccccc7c6cc5n(-c5ccc6ccccc6c5)c4c3)ccc2c1 ; c1ccc(-c2ccc(-n3c4cc(N(c5ccc6ccccc6c5)c5ccc6ccccc6c5)ccc4c4cc5c6ccccc6c6ccccc6c5cc43)cc2)cc1 ; C1=Cc2cccc3c(N(c4ccc5ccccc5c4)c4ccc5c6cc7c8ccccc8c8ccccc8c7cc6n(-c6cccc(-c7ccccc7)c6)c5c4)ccc(c23)C1]

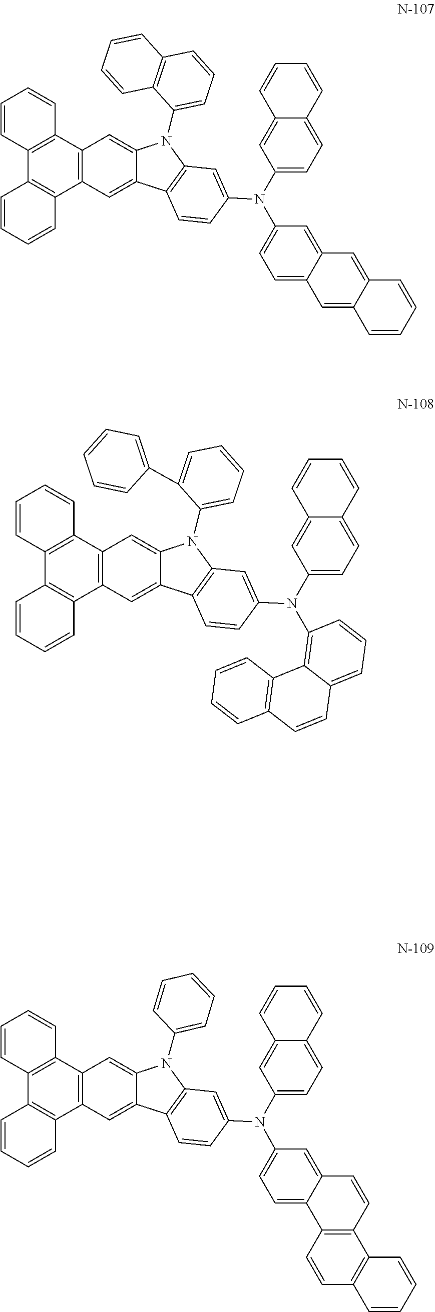 [SMILES: c1ccc2cc(N(c3ccc4cc5ccccc5cc4c3)c3ccc4c5cc6c7ccccc7c7ccccc7c6cc5n(-c5cccc6ccccc56)c4c3)ccc2c1 ; c1ccc(-c2ccccc2-n2c3cc(N(c4ccc5ccccc5c4)c4cccc5ccc6ccccc6c45)ccc3c3cc4c5ccccc5c5ccccc5c4cc32)cc1 ; c1ccc(-n2c3cc(N(c4ccc5ccccc5c4)c4ccc5c(ccc6c7ccccc7ccc56)c4)ccc3c3cc4c5ccccc5c5ccccc5c4cc32)cc1]

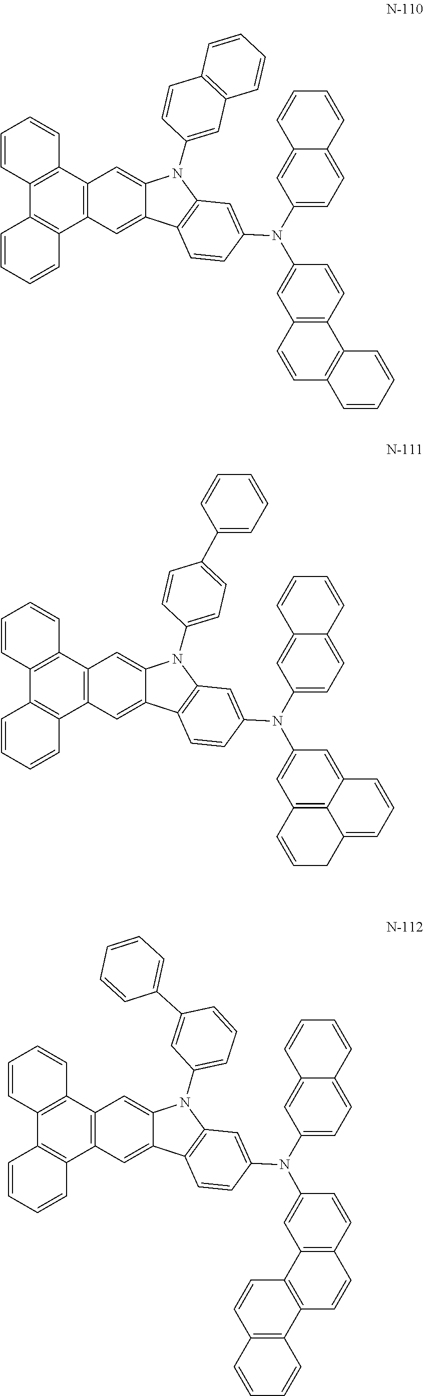 [SMILES: c1ccc2cc(N(c3ccc4c(ccc5ccccc54)c3)c3ccc4c5cc6c7ccccc7c7ccccc7c6cc5n(-c5ccc6ccccc6c5)c4c3)ccc2c1 ; C1=Cc2cc(N(c3ccc4ccccc4c3)c3ccc4c5cc6c7ccccc7c7ccccc7c6cc5n(-c5ccc(-c6ccccc6)cc5)c4c3)cc3cccc(c23)C1 ; c1ccc(-c2cccc(-n3c4cc(N(c5ccc6ccccc6c5)c5ccc6ccc7c8ccccc8ccc7c6c5)ccc4c4cc5c6ccccc6c6ccccc6c5cc43)c2)cc1]

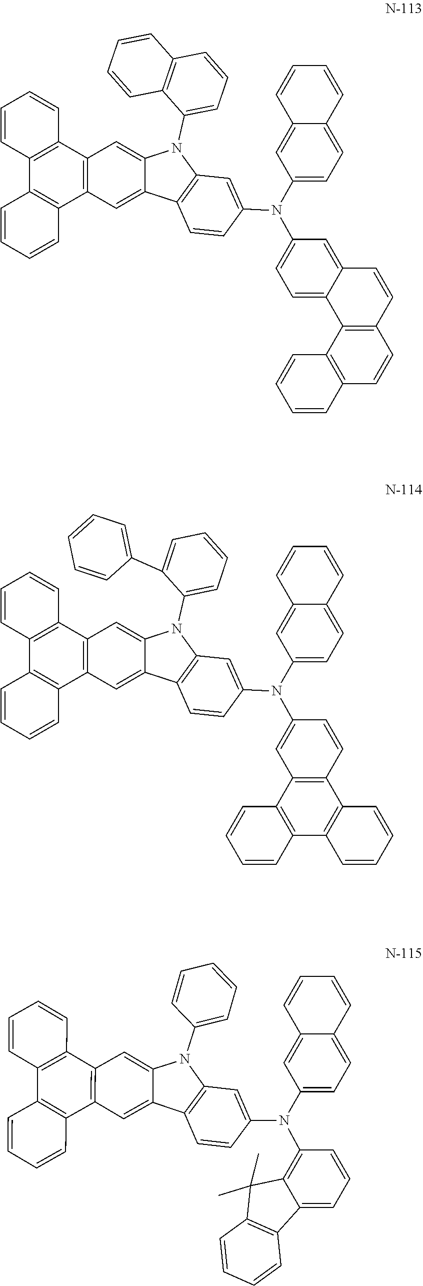 [SMILES: c1ccc2cc(N(c3ccc4c(ccc5ccc6ccccc6c54)c3)c3ccc4c5cc6c7ccccc7c7ccccc7c6cc5n(-c5cccc6ccccc56)c4c3)ccc2c1 ; c1ccc(-c2ccccc2-n2c3cc(N(c4ccc5ccccc5c4)c4ccc5c6ccccc6c6ccccc6c5c4)ccc3c3cc4c5ccccc5c5ccccc5c4cc32)cc1 ; CC1(C)c2ccccc2-c2cccc(N(c3ccc4ccccc4c3)c3ccc4c5cc6c7ccccc7c7ccccc7c6cc5n(-c5ccccc5)c4c3)c21]

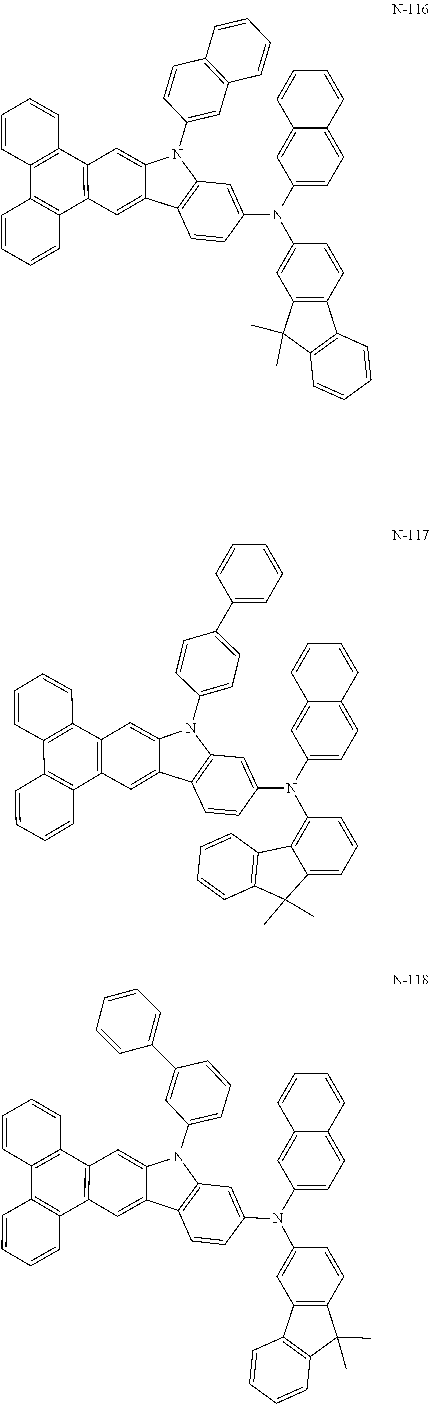 [SMILES: CC1(C)c2ccccc2-c2ccc(N(c3ccc4ccccc4c3)c3ccc4c5cc6c7ccccc7c7ccccc7c6cc5n(-c5ccc6ccccc6c5)c4c3)cc21 ; CC1(C)c2ccccc2-c2c(N(c3ccc4ccccc4c3)c3ccc4c5cc6c7ccccc7c7ccccc7c6cc5n(-c5ccc(-c6ccccc6)cc5)c4c3)cccc21 ; CC1(C)c2ccccc2-c2cc(N(c3ccc4ccccc4c3)c3ccc4c5cc6c7ccccc7c7ccccc7c6cc5n(-c5cccc(-c6ccccc6)c5)c4c3)ccc21]

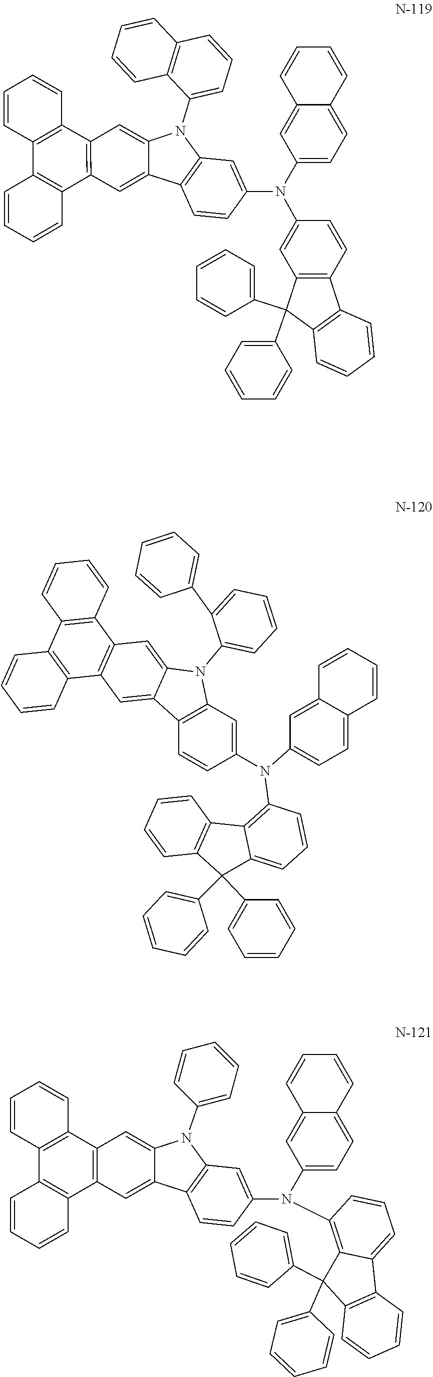 [SMILES: c1ccc(C2(c3ccccc3)c3ccccc3-c3ccc(N(c4ccc5ccccc5c4)c4ccc5c6cc7c8ccccc8c8ccccc8c7cc6n(-c6cccc7ccccc67)c5c4)cc32)cc1 ; c1ccc(-c2ccccc2-n2c3cc(N(c4ccc5ccccc5c4)c4cccc5c4-c4ccccc4C5(c4ccccc4)c4ccccc4)ccc3c3cc4c5ccccc5c5ccccc5c4cc32)cc1 ; c1ccc(-n2c3cc(N(c4ccc5ccccc5c4)c4cccc5c4C(c4ccccc4)(c4ccccc4)c4ccccc4-5)ccc3c3cc4c5ccccc5c5ccccc5c4cc32)cc1]

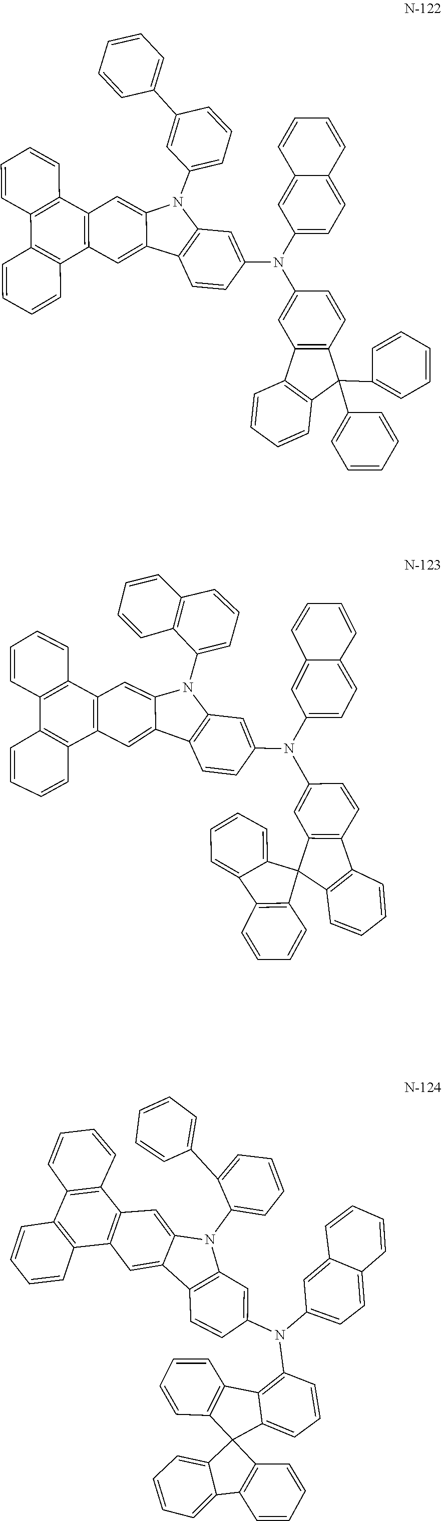 [SMILES: c1ccc(-c2cccc(-n3c4cc(N(c5ccc6c(c5)-c5ccccc5C6(c5ccccc5)c5ccccc5)c5ccc6ccccc6c5)ccc4c4cc5c6ccccc6c6ccccc6c5cc43)c2)cc1 ; c1ccc2c(c1)-c1ccccc1C21c2ccccc2-c2ccc(N(c3ccc4ccccc4c3)c3ccc4c5cc6c7ccccc7c7ccccc7c6cc5n(-c5cccc6ccccc56)c4c3)cc21 ; c1ccc(-c2ccccc2-n2c3cc(N(c4ccc5ccccc5c4)c4cccc5c4-c4ccccc4C54c5ccccc5-c5ccccc54)ccc3c3cc4c5ccccc5c5ccccc5c4cc32)cc1]

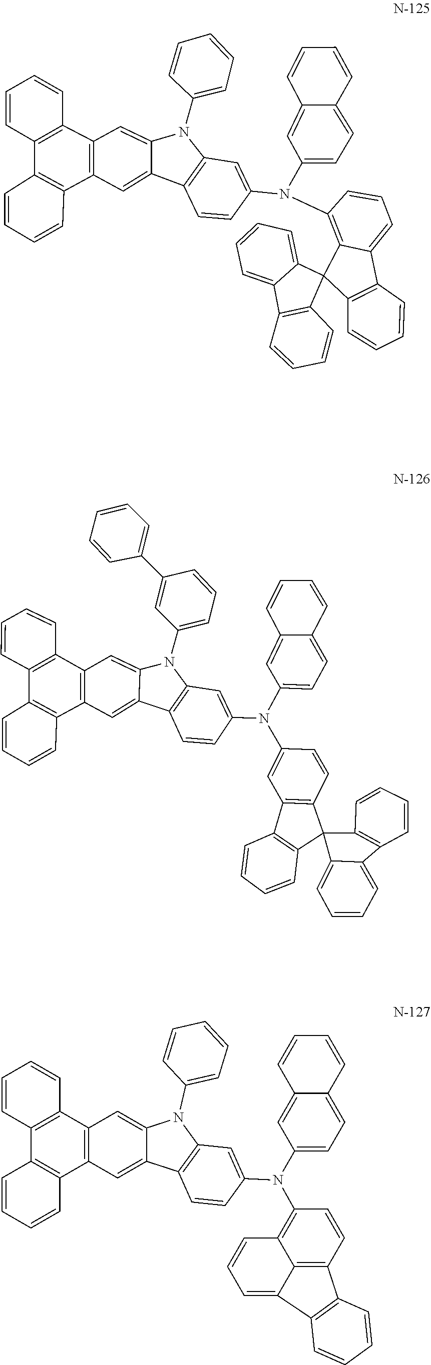 [SMILES: c1ccc(-n2c3cc(N(c4ccc5ccccc5c4)c4cccc5c4C4(c6ccccc6-c6ccccc64)c4ccccc4-5)ccc3c3cc4c5ccccc5c5ccccc5c4cc32)cc1 ; c1ccc(-c2cccc(-n3c4cc(N(c5ccc6c(c5)-c5ccccc5C65c6ccccc6-c6ccccc65)c5ccc6ccccc6c5)ccc4c4cc5c6ccccc6c6ccccc6c5cc43)c2)cc1 ; c1ccc(-n2c3cc(N(c4ccc5ccccc5c4)c4ccc5c6c(cccc46)-c4ccccc4-5)ccc3c3cc4c5ccccc5c5ccccc5c4cc32)cc1]

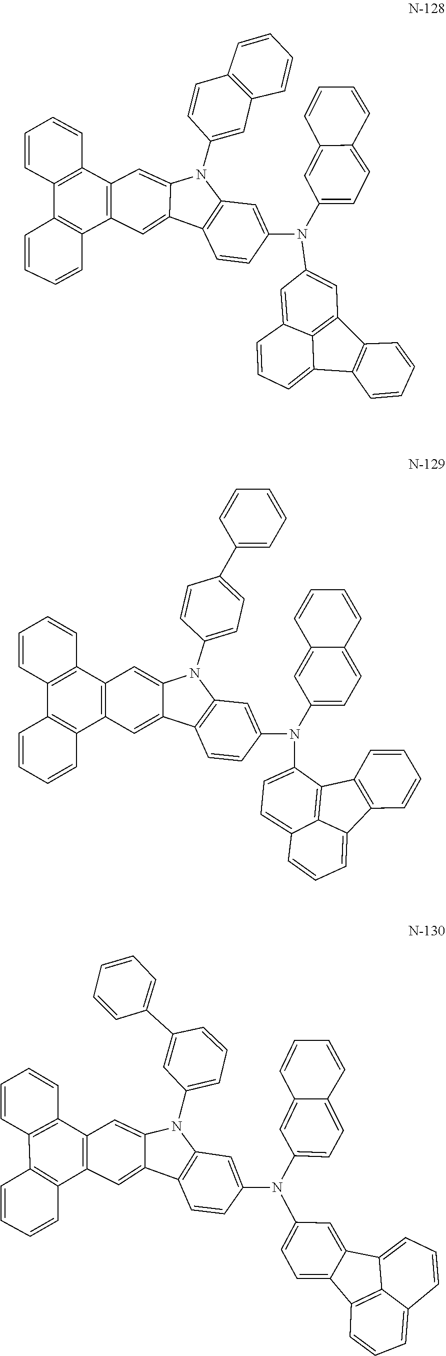 [SMILES: c1ccc2c(c1)-c1cccc3cc(N(c4ccc5ccccc5c4)c4ccc5c6cc7c8ccccc8c8ccccc8c7cc6n(-c6ccc7ccccc7c6)c5c4)cc-2c13 ; c1ccc(-c2ccc(-n3c4cc(N(c5ccc6ccccc6c5)c5ccc6cccc7c6c5-c5ccccc5-7)ccc4c4cc5c6ccccc6c6ccccc6c5cc43)cc2)cc1 ; c1ccc(-c2cccc(-n3c4cc(N(c5ccc6c(c5)-c5cccc7cccc-6c57)c5ccc6ccccc6c5)ccc4c4cc5c6ccccc6c6ccccc6c5cc43)c2)cc1]

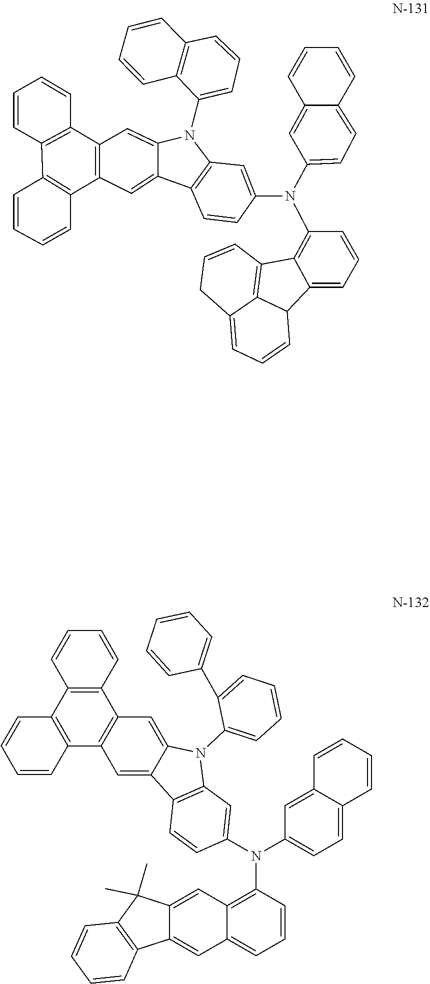 [SMILES: C1=CC2C3=C(C=CCC3=C1)c1c2cccc1N(c1ccc2ccccc2c1)c1ccc2c3cc4c5ccccc5c5ccccc5c4cc3n(-c3cccc4ccccc34)c2c1 ; CC1(C)c2ccccc2-c2cc3cccc(N(c4ccc5ccccc5c4)c4ccc5c6cc7c8ccccc8c8ccccc8c7cc6n(-c6ccccc6-c6ccccc6)c5c4)c3cc21]

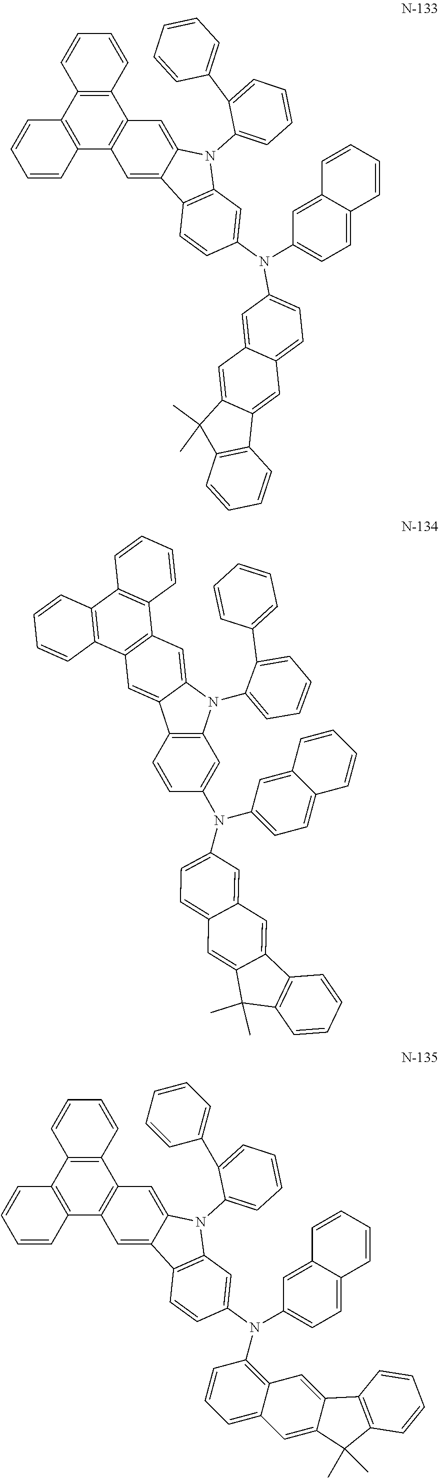 [SMILES: CC1(C)c2ccccc2-c2cc3ccc(N(c4ccc5ccccc5c4)c4ccc5c6cc7c8ccccc8c8ccccc8c7cc6n(-c6ccccc6-c6ccccc6)c5c4)cc3cc21 ; CC1(C)c2ccccc2-c2cc3cc(N(c4ccc5ccccc5c4)c4ccc5c6cc7c8ccccc8c8ccccc8c7cc6n(-c6ccccc6-c6ccccc6)c5c4)ccc3cc21 ; CC1(C)c2ccccc2-c2cc3c(N(c4ccc5ccccc5c4)c4ccc5c6cc7c8ccccc8c8ccccc8c7cc6n(-c6ccccc6-c6ccccc6)c5c4)cccc3cc21]

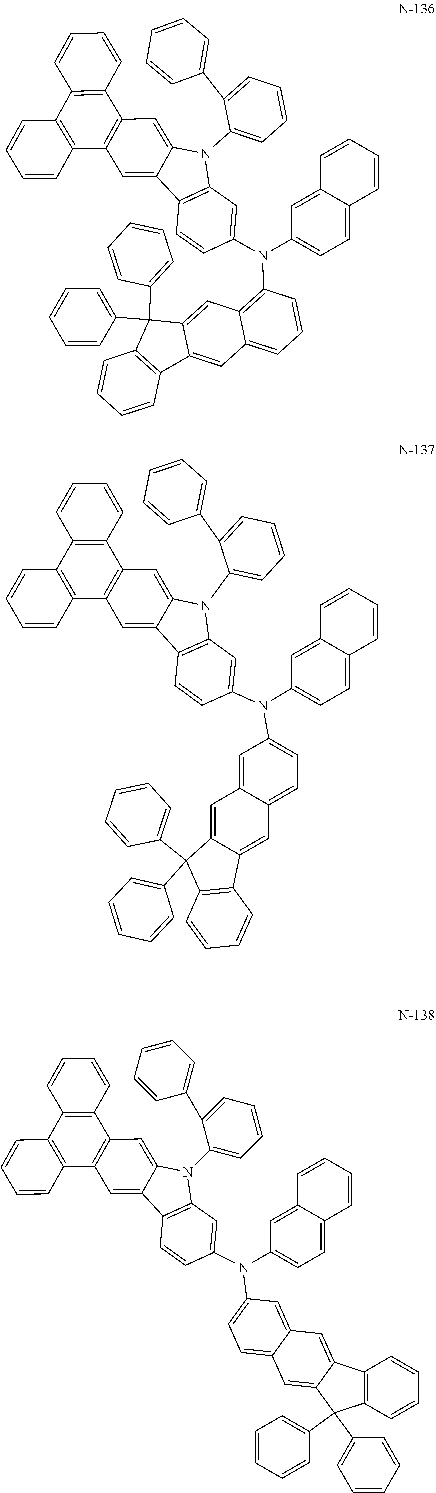 [SMILES: c1ccc(-c2ccccc2-n2c3cc(N(c4ccc5ccccc5c4)c4cccc5cc6c(cc45)C(c4ccccc4)(c4ccccc4)c4ccccc4-6)ccc3c3cc4c5ccccc5c5ccccc5c4cc32)cc1 ; c1ccc(-c2ccccc2-n2c3cc(N(c4ccc5ccccc5c4)c4ccc5cc6c(cc5c4)C(c4ccccc4)(c4ccccc4)c4ccccc4-6)ccc3c3cc4c5ccccc5c5ccccc5c4cc32)cc1 ; c1ccc(-c2ccccc2-n2c3cc(N(c4ccc5ccccc5c4)c4ccc5cc6c(cc5c4)-c4ccccc4C6(c4ccccc4)c4ccccc4)ccc3c3cc4c5ccccc5c5ccccc5c4cc32)cc1]

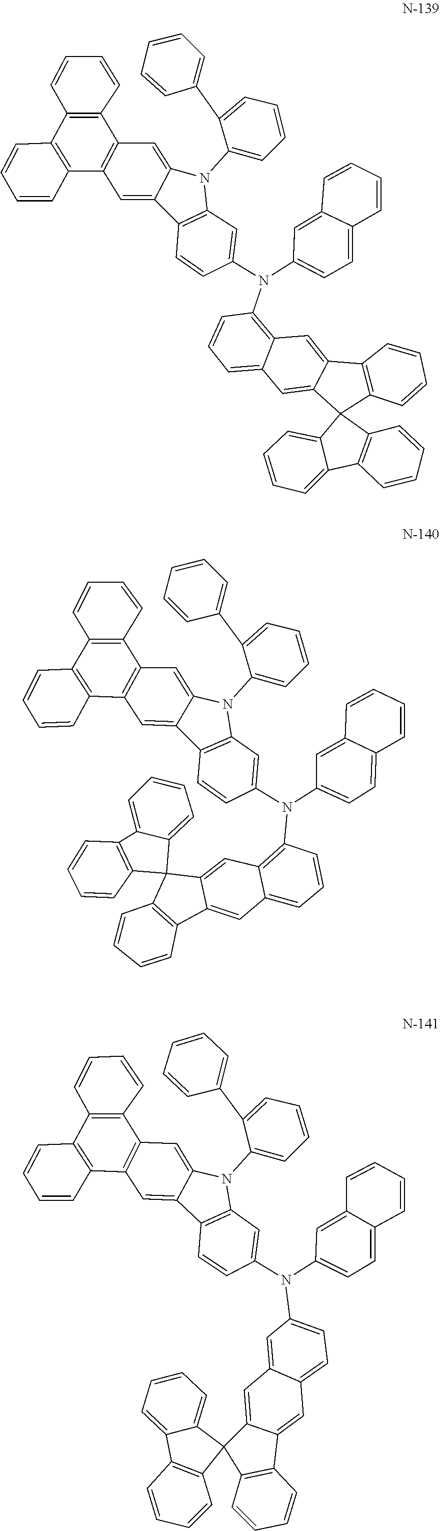 [SMILES: c1ccc(-c2ccccc2-n2c3cc(N(c4ccc5ccccc5c4)c4cccc5cc6c(cc45)-c4ccccc4C64c5ccccc5-c5ccccc54)ccc3c3cc4c5ccccc5c5ccccc5c4cc32)cc1 ; c1ccc(-c2ccccc2-n2c3cc(N(c4ccc5ccccc5c4)c4cccc5cc6c(cc45)C4(c5ccccc5-c5ccccc54)c4ccccc4-6)ccc3c3cc4c5ccccc5c5ccccc5c4cc32)cc1 ; c1ccc(-c2ccccc2-n2c3cc(N(c4ccc5ccccc5c4)c4ccc5cc6c(cc5c4)C4(c5ccccc5-c5ccccc54)c4ccccc4-6)ccc3c3cc4c5ccccc5c5ccccc5c4cc32)cc1]

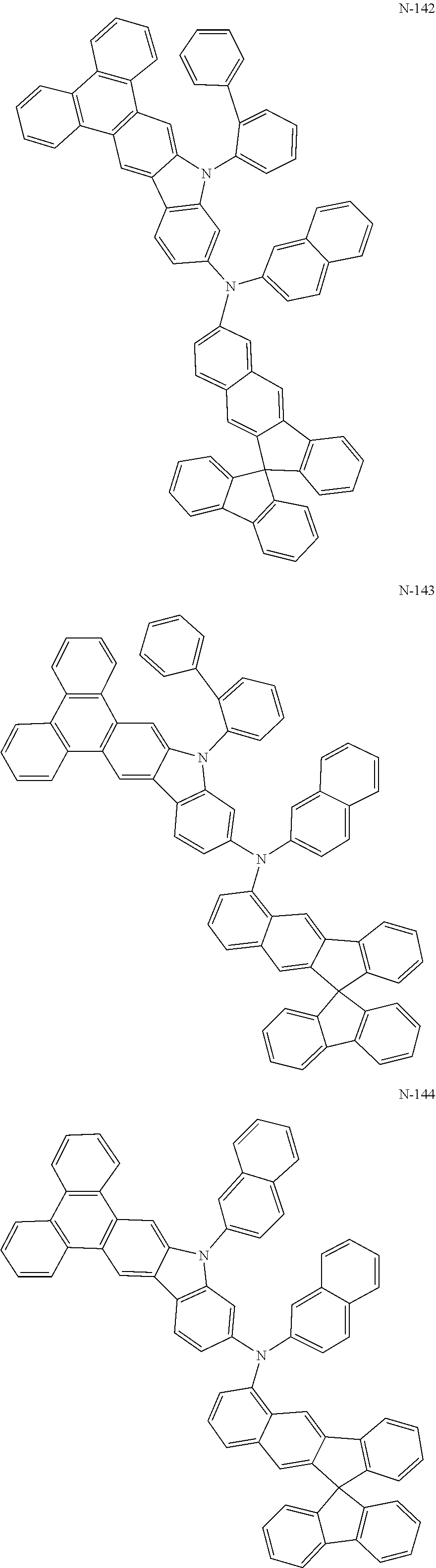 [SMILES: c1ccc(-c2ccccc2-n2c3cc(N(c4ccc5ccccc5c4)c4ccc5cc6c(cc5c4)-c4ccccc4C64c5ccccc5-c5ccccc54)ccc3c3cc4c5ccccc5c5ccccc5c4cc32)cc1 ; c1ccc(-c2ccccc2-n2c3cc(N(c4ccc5ccccc5c4)c4cccc5cc6c(cc45)-c4ccccc4C64c5ccccc5-c5ccccc54)ccc3c3cc4c5ccccc5c5ccccc5c4cc32)cc1 ; c1ccc2c(c1)-c1ccccc1C21c2ccccc2-c2cc3c(N(c4ccc5ccccc5c4)c4ccc5c6cc7c8ccccc8c8ccccc8c7cc6n(-c6ccc7ccccc7c6)c5c4)cccc3cc21]

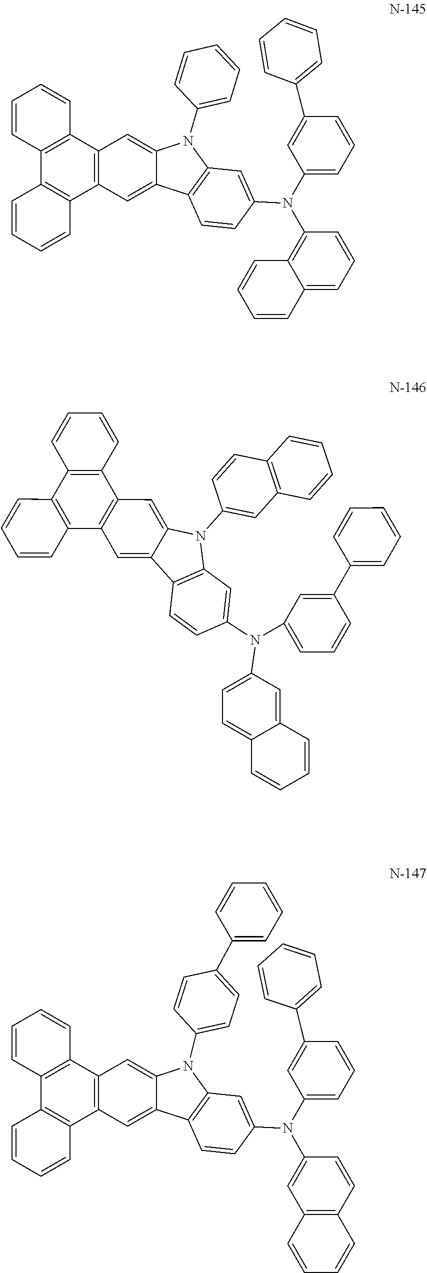 [SMILES: c1ccc(-c2cccc(N(c3ccc4c5cc6c7ccccc7c7ccccc7c6cc5n(-c5ccccc5)c4c3)c3cccc4ccccc34)c2)cc1 ; c1ccc(-c2cccc(N(c3ccc4ccccc4c3)c3ccc4c5cc6c7ccccc7c7ccccc7c6cc5n(-c5ccc6ccccc6c5)c4c3)c2)cc1 ; c1ccc(-c2ccc(-n3c4cc(N(c5cccc(-c6ccccc6)c5)c5ccc6ccccc6c5)ccc4c4cc5c6ccccc6c6ccccc6c5cc43)cc2)cc1]

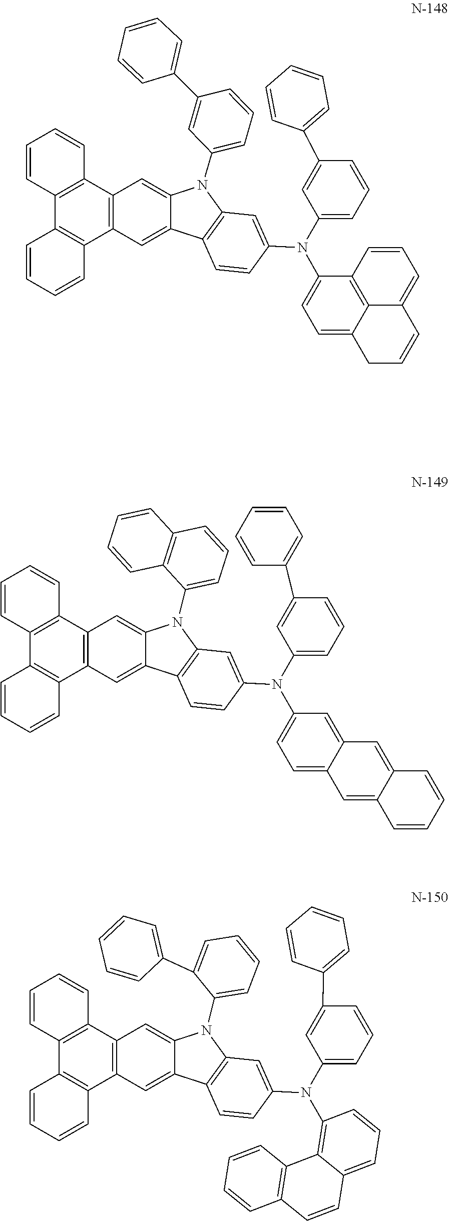 [SMILES: C1=Cc2cccc3c(N(c4cccc(-c5ccccc5)c4)c4ccc5c6cc7c8ccccc8c8ccccc8c7cc6n(-c6cccc(-c7ccccc7)c6)c5c4)ccc(c23)C1 ; c1ccc(-c2cccc(N(c3ccc4cc5ccccc5cc4c3)c3ccc4c5cc6c7ccccc7c7ccccc7c6cc5n(-c5cccc6ccccc56)c4c3)c2)cc1 ; c1ccc(-c2cccc(N(c3ccc4c5cc6c7ccccc7c7ccccc7c6cc5n(-c5ccccc5-c5ccccc5)c4c3)c3cccc4ccc5ccccc5c34)c2)cc1]

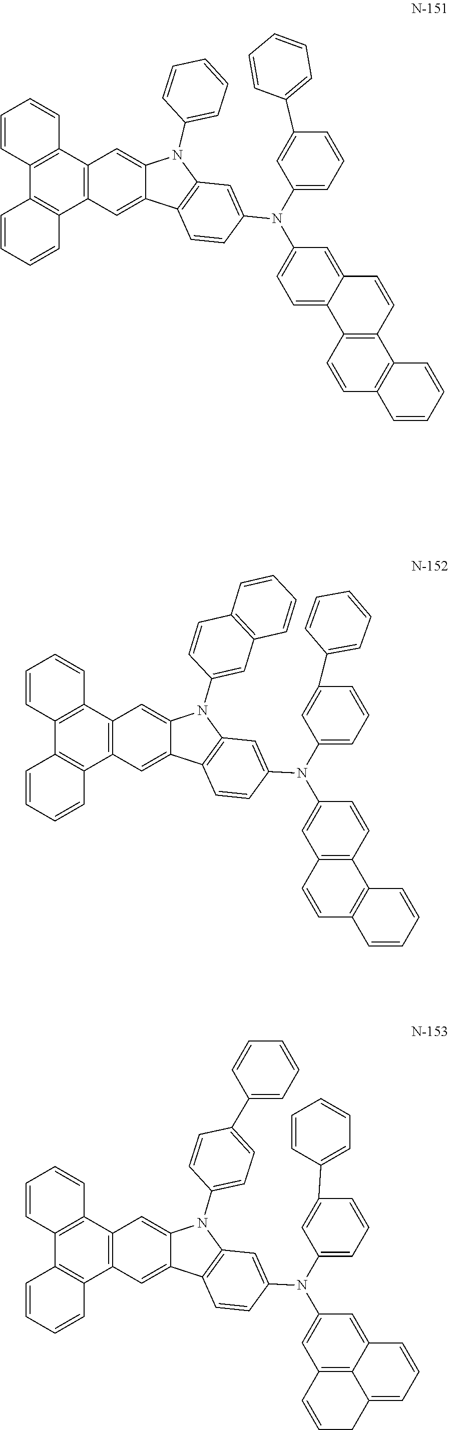 [SMILES: c1ccc(-c2cccc(N(c3ccc4c(ccc5c6ccccc6ccc45)c3)c3ccc4c5cc6c7ccccc7c7ccccc7c6cc5n(-c5ccccc5)c4c3)c2)cc1 ; c1ccc(-c2cccc(N(c3ccc4c(ccc5ccccc54)c3)c3ccc4c5cc6c7ccccc7c7ccccc7c6cc5n(-c5ccc6ccccc6c5)c4c3)c2)cc1 ; C1=Cc2cc(N(c3cccc(-c4ccccc4)c3)c3ccc4c5cc6c7ccccc7c7ccccc7c6cc5n(-c5ccc(-c6ccccc6)cc5)c4c3)cc3cccc(c23)C1]

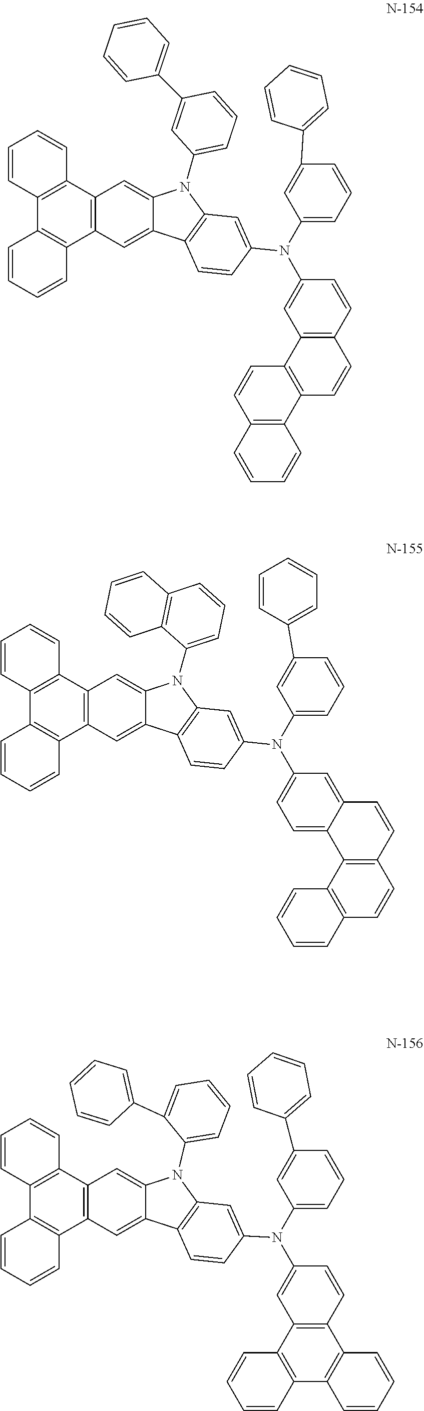 [SMILES: c1ccc(-c2cccc(N(c3ccc4ccc5c6ccccc6ccc5c4c3)c3ccc4c5cc6c7ccccc7c7ccccc7c6cc5n(-c5cccc(-c6ccccc6)c5)c4c3)c2)cc1 ; c1ccc(-c2cccc(N(c3ccc4c(ccc5ccc6ccccc6c54)c3)c3ccc4c5cc6c7ccccc7c7ccccc7c6cc5n(-c5cccc6ccccc56)c4c3)c2)cc1 ; c1ccc(-c2cccc(N(c3ccc4c5ccccc5c5ccccc5c4c3)c3ccc4c5cc6c7ccccc7c7ccccc7c6cc5n(-c5ccccc5-c5ccccc5)c4c3)c2)cc1]

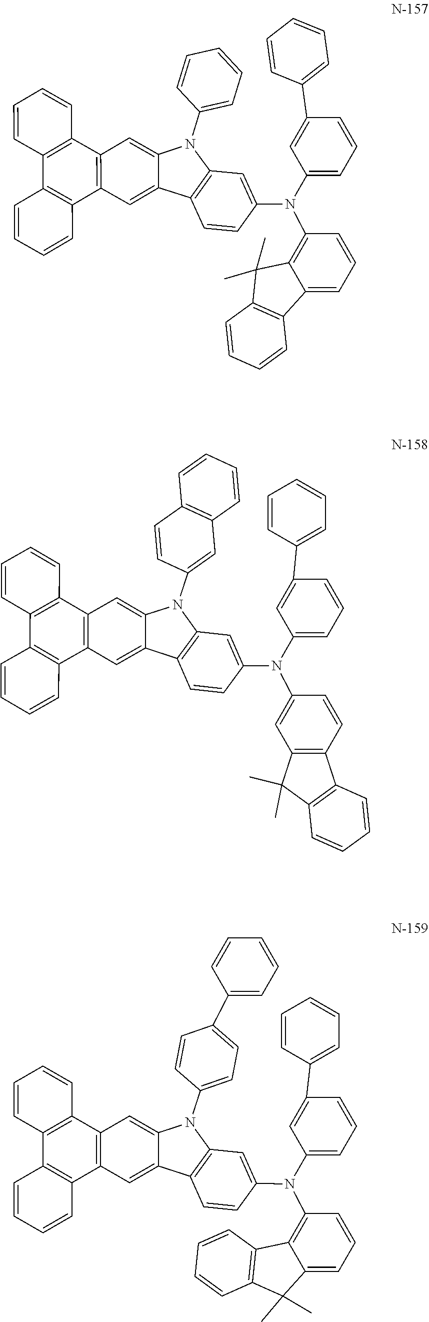 [SMILES: CC1(C)c2ccccc2-c2cccc(N(c3cccc(-c4ccccc4)c3)c3ccc4c5cc6c7ccccc7c7ccccc7c6cc5n(-c5ccccc5)c4c3)c21 ; CC1(C)c2ccccc2-c2ccc(N(c3cccc(-c4ccccc4)c3)c3ccc4c5cc6c7ccccc7c7ccccc7c6cc5n(-c5ccc6ccccc6c5)c4c3)cc21 ; CC1(C)c2ccccc2-c2c(N(c3cccc(-c4ccccc4)c3)c3ccc4c5cc6c7ccccc7c7ccccc7c6cc5n(-c5ccc(-c6ccccc6)cc5)c4c3)cccc21]

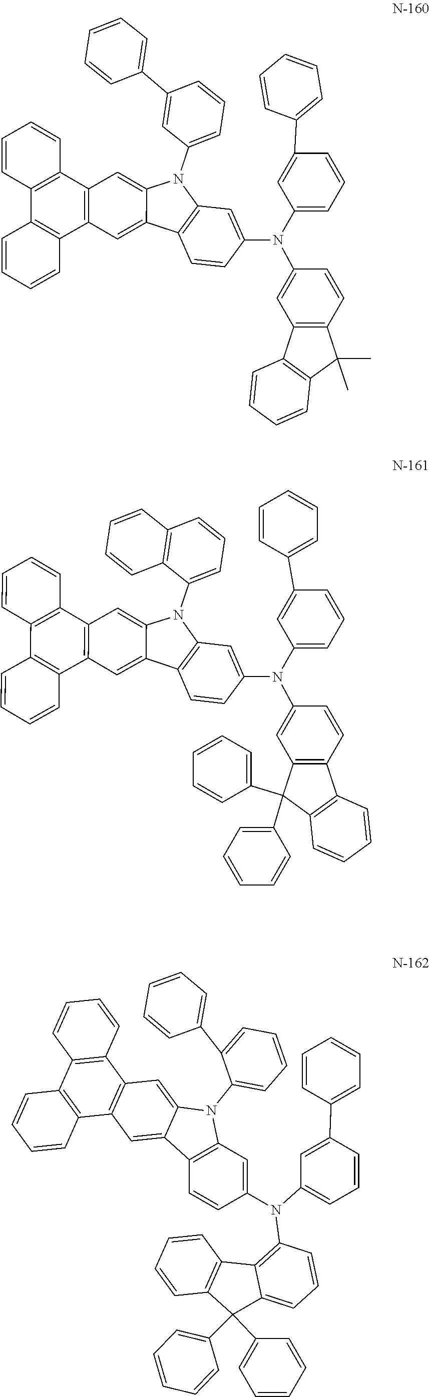 [SMILES: CC1(C)c2ccccc2-c2cc(N(c3cccc(-c4ccccc4)c3)c3ccc4c5cc6c7ccccc7c7ccccc7c6cc5n(-c5cccc(-c6ccccc6)c5)c4c3)ccc21 ; c1ccc(-c2cccc(N(c3ccc4c(c3)C(c3ccccc3)(c3ccccc3)c3ccccc3-4)c3ccc4c5cc6c7ccccc7c7ccccc7c6cc5n(-c5cccc6ccccc56)c4c3)c2)cc1 ; c1ccc(-c2cccc(N(c3ccc4c5cc6c7ccccc7c7ccccc7c6cc5n(-c5ccccc5-c5ccccc5)c4c3)c3cccc4c3-c3ccccc3C4(c3ccccc3)c3ccccc3)c2)cc1]

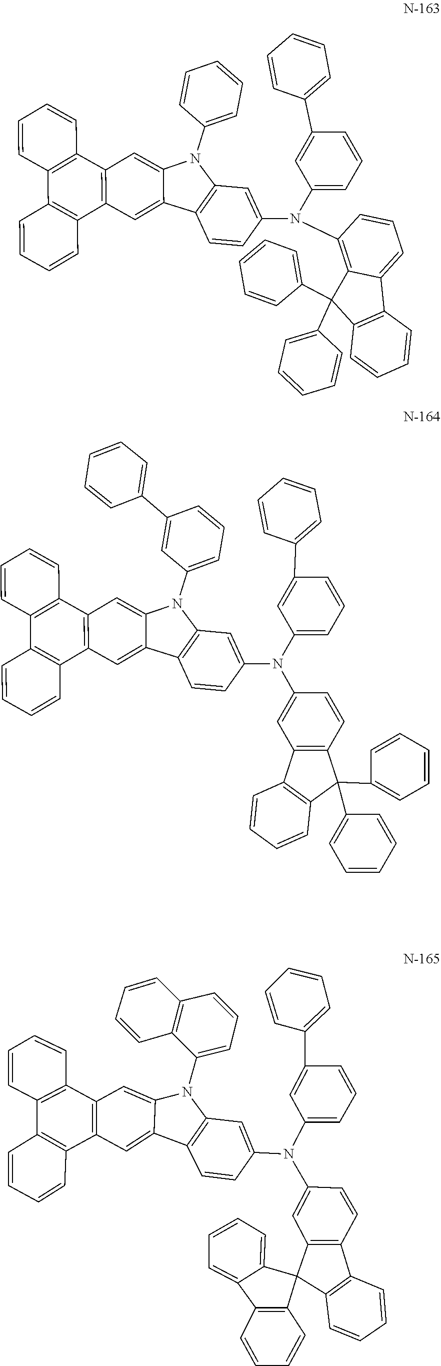 [SMILES: c1ccc(-c2cccc(N(c3ccc4c5cc6c7ccccc7c7ccccc7c6cc5n(-c5ccccc5)c4c3)c3cccc4c3C(c3ccccc3)(c3ccccc3)c3ccccc3-4)c2)cc1 ; c1ccc(-c2cccc(N(c3ccc4c(c3)-c3ccccc3C4(c3ccccc3)c3ccccc3)c3ccc4c5cc6c7ccccc7c7ccccc7c6cc5n(-c5cccc(-c6ccccc6)c5)c4c3)c2)cc1 ; c1ccc(-c2cccc(N(c3ccc4c(c3)C3(c5ccccc5-c5ccccc53)c3ccccc3-4)c3ccc4c5cc6c7ccccc7c7ccccc7c6cc5n(-c5cccc6ccccc56)c4c3)c2)cc1]

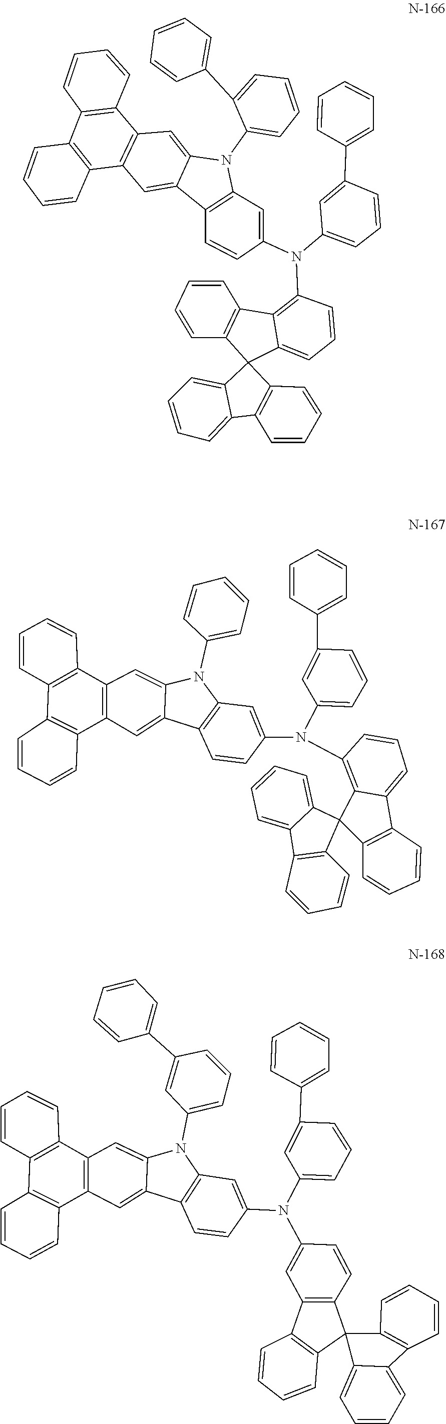 [SMILES: c1ccc(-c2cccc(N(c3ccc4c5cc6c7ccccc7c7ccccc7c6cc5n(-c5ccccc5-c5ccccc5)c4c3)c3cccc4c3-c3ccccc3C43c4ccccc4-c4ccccc43)c2)cc1 ; c1ccc(-c2cccc(N(c3ccc4c5cc6c7ccccc7c7ccccc7c6cc5n(-c5ccccc5)c4c3)c3cccc4c3C3(c5ccccc5-c5ccccc53)c3ccccc3-4)c2)cc1 ; c1ccc(-c2cccc(N(c3ccc4c(c3)-c3ccccc3C43c4ccccc4-c4ccccc43)c3ccc4c5cc6c7ccccc7c7ccccc7c6cc5n(-c5cccc(-c6ccccc6)c5)c4c3)c2)cc1]

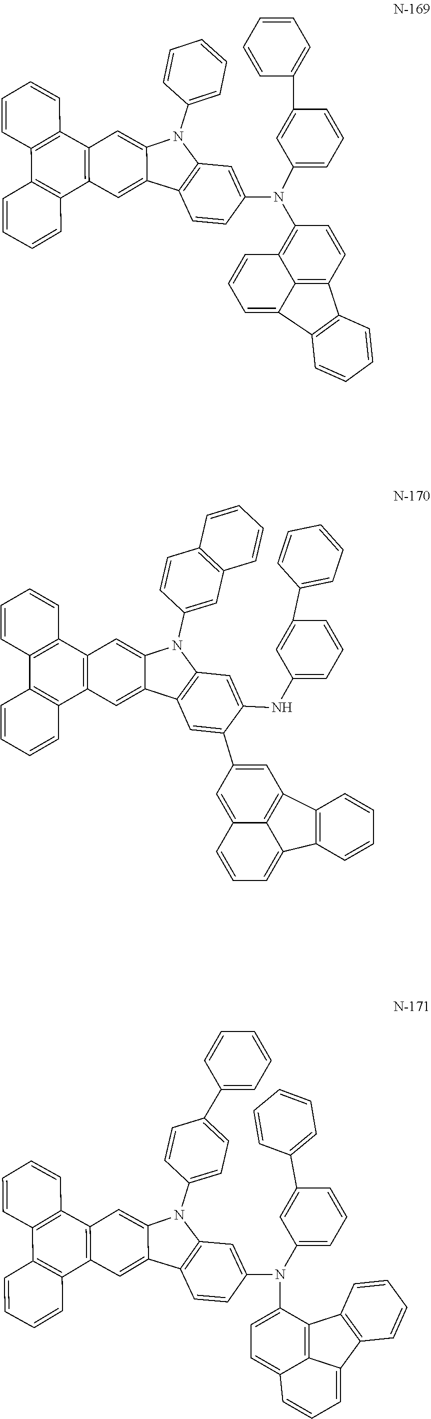 [SMILES: c1ccc(-c2cccc(N(c3ccc4c5cc6c7ccccc7c7ccccc7c6cc5n(-c5ccccc5)c4c3)c3ccc4c5c(cccc35)-c3ccccc3-4)c2)cc1 ; c1ccc(-c2cccc(Nc3cc4c(cc3-c3cc5c6c(cccc6c3)-c3ccccc3-5)c3cc5c6ccccc6c6ccccc6c5cc3n4-c3ccc4ccccc4c3)c2)cc1 ; c1ccc(-c2ccc(-n3c4cc(N(c5cccc(-c6ccccc6)c5)c5ccc6cccc7c6c5-c5ccccc5-7)ccc4c4cc5c6ccccc6c6ccccc6c5cc43)cc2)cc1]

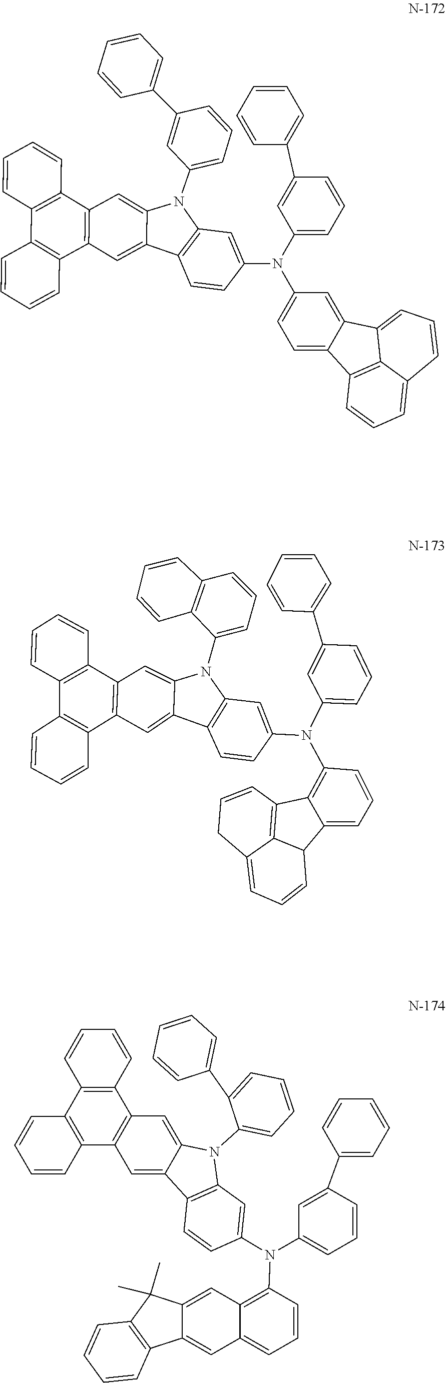 [SMILES: c1ccc(-c2cccc(N(c3ccc4c(c3)-c3cccc5cccc-4c35)c3ccc4c5cc6c7ccccc7c7ccccc7c6cc5n(-c5cccc(-c6ccccc6)c5)c4c3)c2)cc1 ; C1=CC2C3=C(C=CCC3=C1)c1c2cccc1N(c1cccc(-c2ccccc2)c1)c1ccc2c3cc4c5ccccc5c5ccccc5c4cc3n(-c3cccc4ccccc34)c2c1 ; CC1(C)c2ccccc2-c2cc3cccc(N(c4cccc(-c5ccccc5)c4)c4ccc5c6cc7c8ccccc8c8ccccc8c7cc6n(-c6ccccc6-c6ccccc6)c5c4)c3cc21]

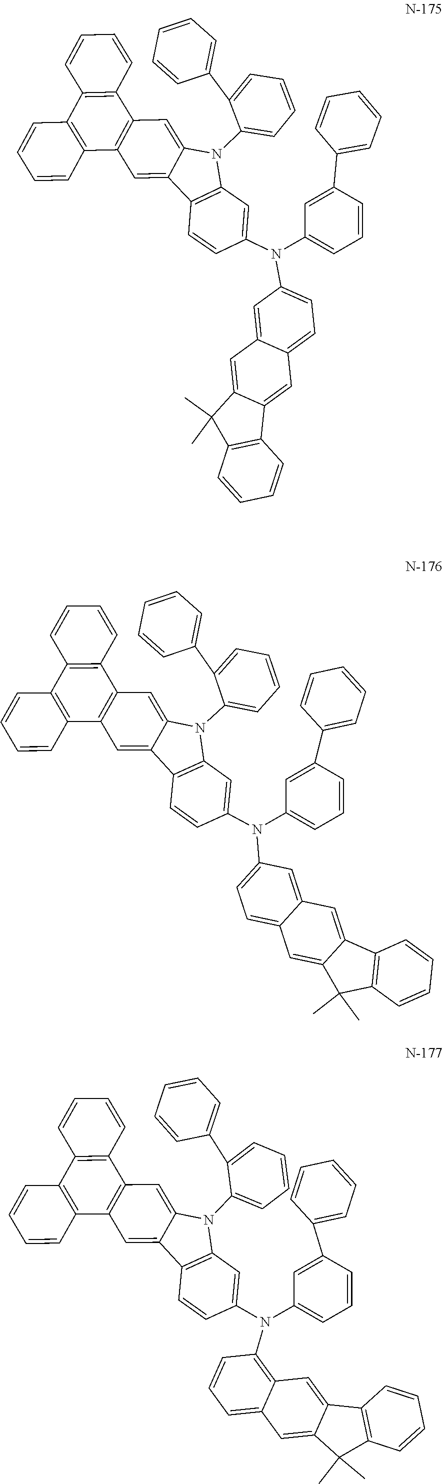 [SMILES: CC1(C)c2ccccc2-c2cc3ccc(N(c4cccc(-c5ccccc5)c4)c4ccc5c6cc7c8ccccc8c8ccccc8c7cc6n(-c6ccccc6-c6ccccc6)c5c4)cc3cc21 ; CC1(C)c2ccccc2-c2cc3cc(N(c4cccc(-c5ccccc5)c4)c4ccc5c6cc7c8ccccc8c8ccccc8c7cc6n(-c6ccccc6-c6ccccc6)c5c4)ccc3cc21 ; CC1(C)c2ccccc2-c2cc3c(N(c4cccc(-c5ccccc5)c4)c4ccc5c6cc7c8ccccc8c8ccccc8c7cc6n(-c6ccccc6-c6ccccc6)c5c4)cccc3cc21]

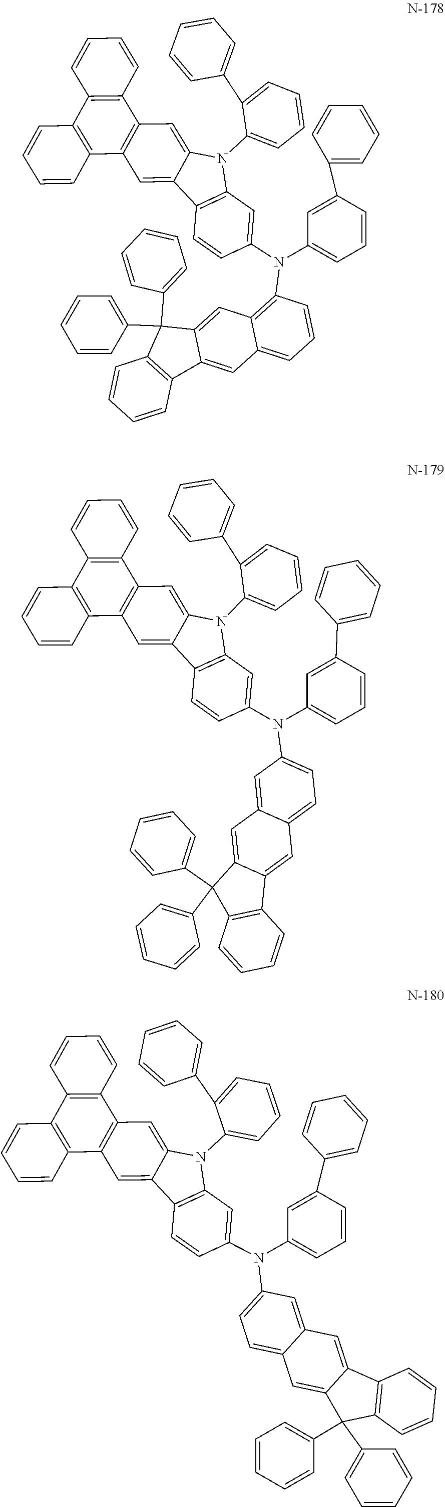 [SMILES: c1ccc(-c2cccc(N(c3ccc4c5cc6c7ccccc7c7ccccc7c6cc5n(-c5ccccc5-c5ccccc5)c4c3)c3cccc4cc5c(cc34)C(c3ccccc3)(c3ccccc3)c3ccccc3-5)c2)cc1 ; c1ccc(-c2cccc(N(c3ccc4cc5c(cc4c3)C(c3ccccc3)(c3ccccc3)c3ccccc3-5)c3ccc4c5cc6c7ccccc7c7ccccc7c6cc5n(-c5ccccc5-c5ccccc5)c4c3)c2)cc1 ; c1ccc(-c2cccc(N(c3ccc4cc5c(cc4c3)-c3ccccc3C5(c3ccccc3)c3ccccc3)c3ccc4c5cc6c7ccccc7c7ccccc7c6cc5n(-c5ccccc5-c5ccccc5)c4c3)c2)cc1]

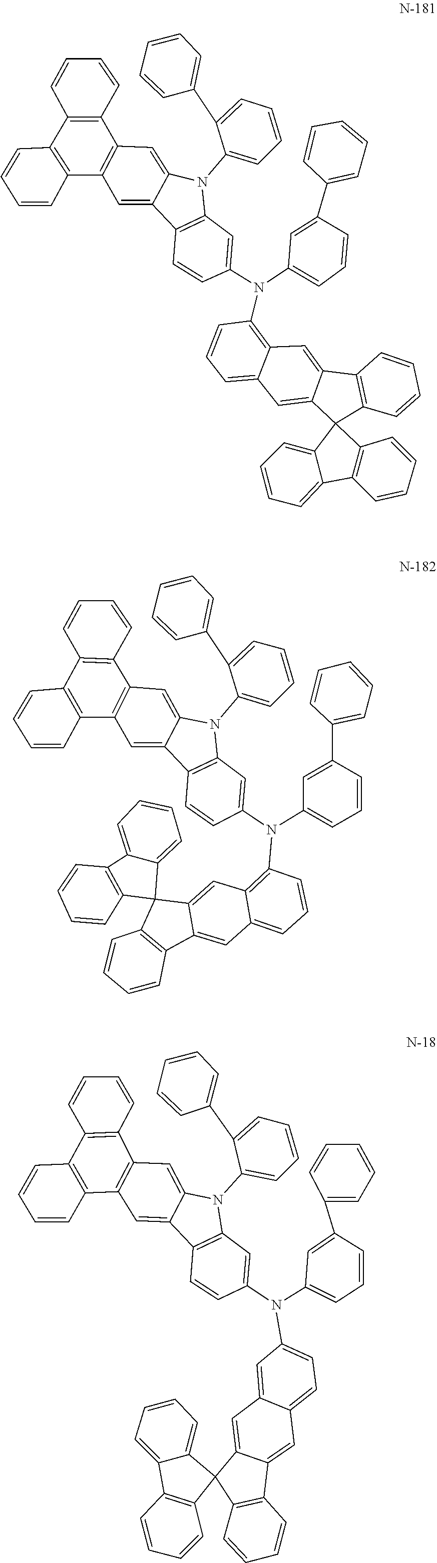 [SMILES: c1ccc(-c2cccc(N(c3ccc4c5cc6c7ccccc7c7ccccc7c6cc5n(-c5ccccc5-c5ccccc5)c4c3)c3cccc4cc5c(cc34)-c3ccccc3C53c4ccccc4-c4ccccc43)c2)cc1 ; c1ccc(-c2cccc(N(c3ccc4c5cc6c7ccccc7c7ccccc7c6cc5n(-c5ccccc5-c5ccccc5)c4c3)c3cccc4cc5c(cc34)C3(c4ccccc4-c4ccccc43)c3ccccc3-5)c2)cc1 ; c1ccc(-c2cccc(N(c3ccc4cc5c(cc4c3)C3(c4ccccc4-c4ccccc43)c3ccccc3-5)c3ccc4c5cc6c7ccccc7c7ccccc7c6cc5n(-c5ccccc5-c5ccccc5)c4c3)c2)cc1]

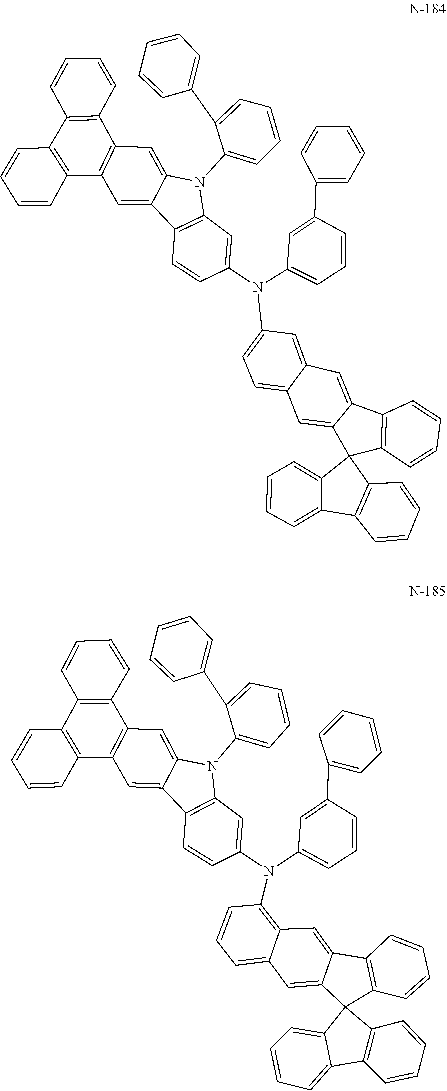 [SMILES: c1ccc(-c2cccc(N(c3ccc4cc5c(cc4c3)-c3ccccc3C53c4ccccc4-c4ccccc43)c3ccc4c5cc6c7ccccc7c7ccccc7c6cc5n(-c5ccccc5-c5ccccc5)c4c3)c2)cc1 ; c1ccc(-c2cccc(N(c3ccc4c5cc6c7ccccc7c7ccccc7c6cc5n(-c5ccccc5-c5ccccc5)c4c3)c3cccc4cc5c(cc34)-c3ccccc3C53c4ccccc4-c4ccccc43)c2)cc1]

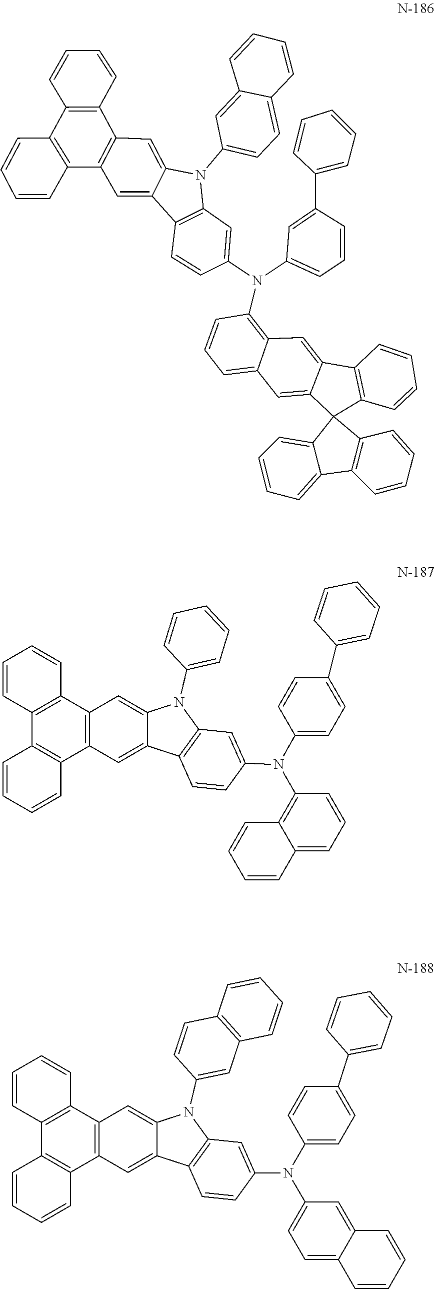 [SMILES: c1ccc(-c2cccc(N(c3ccc4c5cc6c7ccccc7c7ccccc7c6cc5n(-c5ccc6ccccc6c5)c4c3)c3cccc4cc5c(cc34)-c3ccccc3C53c4ccccc4-c4ccccc43)c2)cc1 ; c1ccc(-c2ccc(N(c3ccc4c5cc6c7ccccc7c7ccccc7c6cc5n(-c5ccccc5)c4c3)c3cccc4ccccc34)cc2)cc1 ; c1ccc(-c2ccc(N(c3ccc4ccccc4c3)c3ccc4c5cc6c7ccccc7c7ccccc7c6cc5n(-c5ccc6ccccc6c5)c4c3)cc2)cc1]

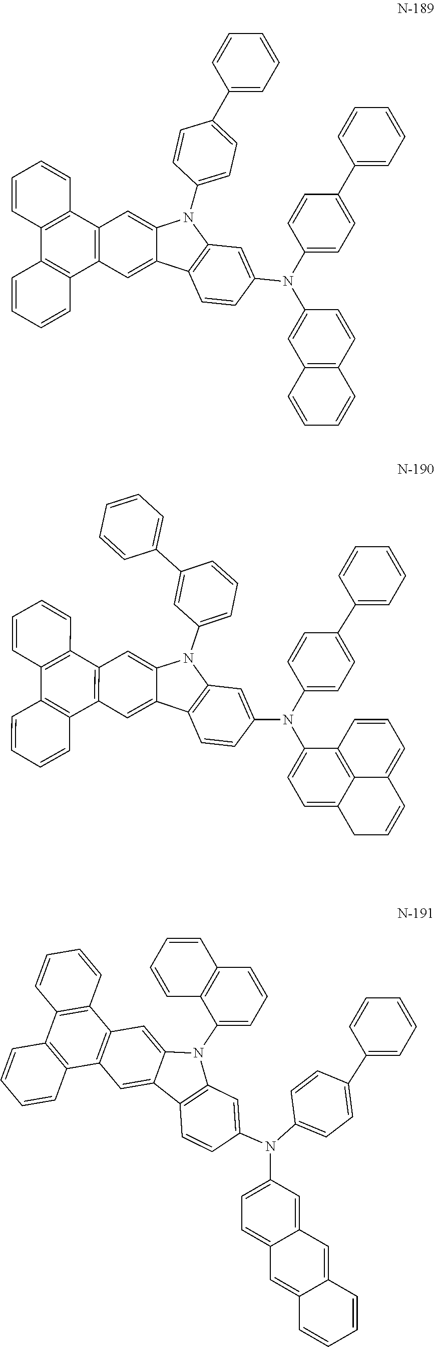 [SMILES: c1ccc(-c2ccc(N(c3ccc4ccccc4c3)c3ccc4c5cc6c7ccccc7c7ccccc7c6cc5n(-c5ccc(-c6ccccc6)cc5)c4c3)cc2)cc1 ; C1=Cc2cccc3c(N(c4ccc(-c5ccccc5)cc4)c4ccc5c6cc7c8ccccc8c8ccccc8c7cc6n(-c6cccc(-c7ccccc7)c6)c5c4)ccc(c23)C1 ; c1ccc(-c2ccc(N(c3ccc4cc5ccccc5cc4c3)c3ccc4c5cc6c7ccccc7c7ccccc7c6cc5n(-c5cccc6ccccc56)c4c3)cc2)cc1]

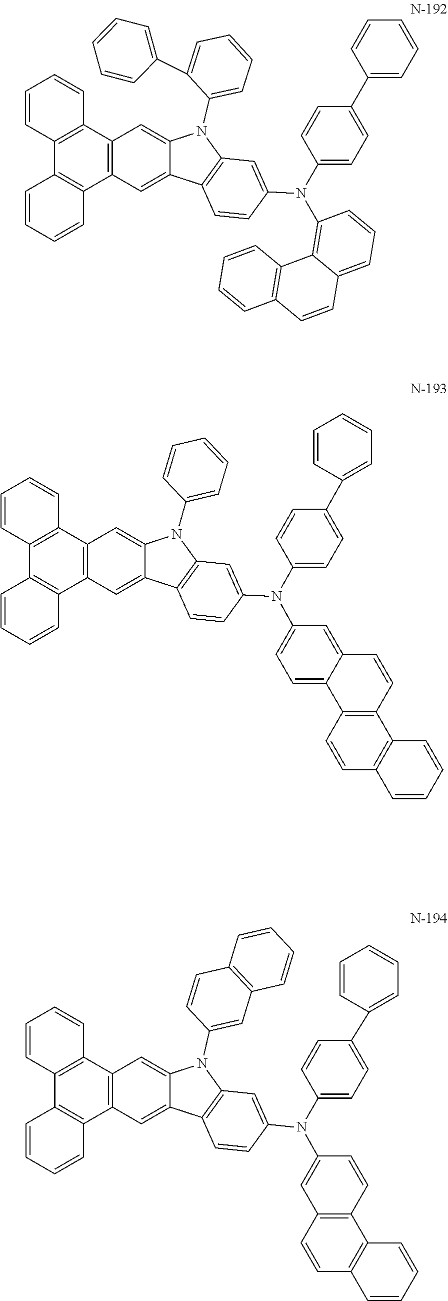 [SMILES: c1ccc(-c2ccc(N(c3ccc4c5cc6c7ccccc7c7ccccc7c6cc5n(-c5ccccc5-c5ccccc5)c4c3)c3cccc4ccc5ccccc5c34)cc2)cc1 ; c1ccc(-c2ccc(N(c3ccc4c(ccc5c6ccccc6ccc45)c3)c3ccc4c5cc6c7ccccc7c7ccccc7c6cc5n(-c5ccccc5)c4c3)cc2)cc1 ; c1ccc(-c2ccc(N(c3ccc4c(ccc5ccccc54)c3)c3ccc4c5cc6c7ccccc7c7ccccc7c6cc5n(-c5ccc6ccccc6c5)c4c3)cc2)cc1]

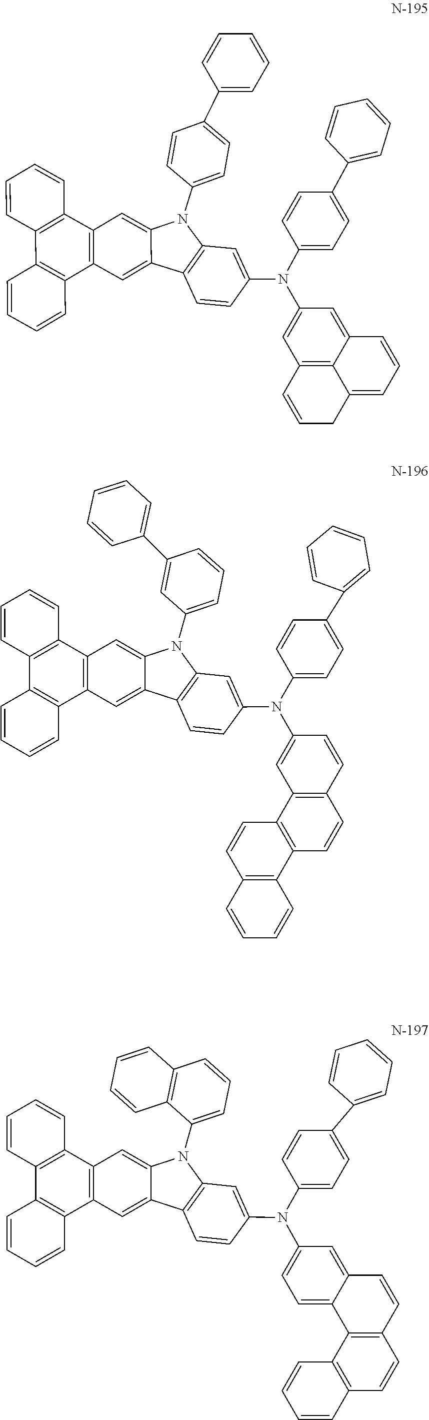 [SMILES: C1=Cc2cc(N(c3ccc(-c4ccccc4)cc3)c3ccc4c5cc6c7ccccc7c7ccccc7c6cc5n(-c5ccc(-c6ccccc6)cc5)c4c3)cc3cccc(c23)C1 ; c1ccc(-c2ccc(N(c3ccc4ccc5c6ccccc6ccc5c4c3)c3ccc4c5cc6c7ccccc7c7ccccc7c6cc5n(-c5cccc(-c6ccccc6)c5)c4c3)cc2)cc1 ; c1ccc(-c2ccc(N(c3ccc4c(ccc5ccc6ccccc6c54)c3)c3ccc4c5cc6c7ccccc7c7ccccc7c6cc5n(-c5cccc6ccccc56)c4c3)cc2)cc1]

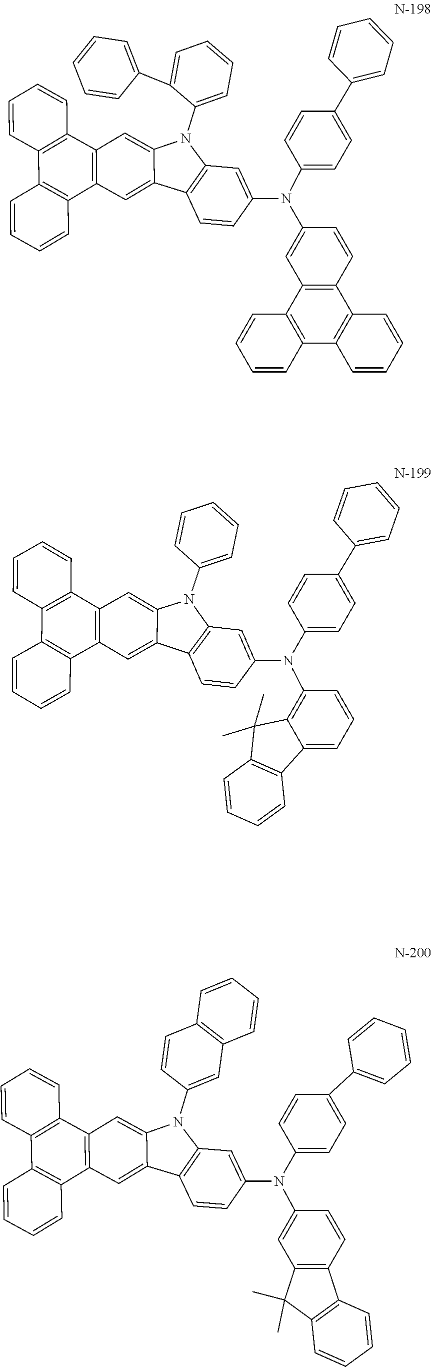 [SMILES: c1ccc(-c2ccc(N(c3ccc4c5ccccc5c5ccccc5c4c3)c3ccc4c5cc6c7ccccc7c7ccccc7c6cc5n(-c5ccccc5-c5ccccc5)c4c3)cc2)cc1 ; CC1(C)c2ccccc2-c2cccc(N(c3ccc(-c4ccccc4)cc3)c3ccc4c5cc6c7ccccc7c7ccccc7c6cc5n(-c5ccccc5)c4c3)c21 ; CC1(C)c2ccccc2-c2ccc(N(c3ccc(-c4ccccc4)cc3)c3ccc4c5cc6c7ccccc7c7ccccc7c6cc5n(-c5ccc6ccccc6c5)c4c3)cc21]

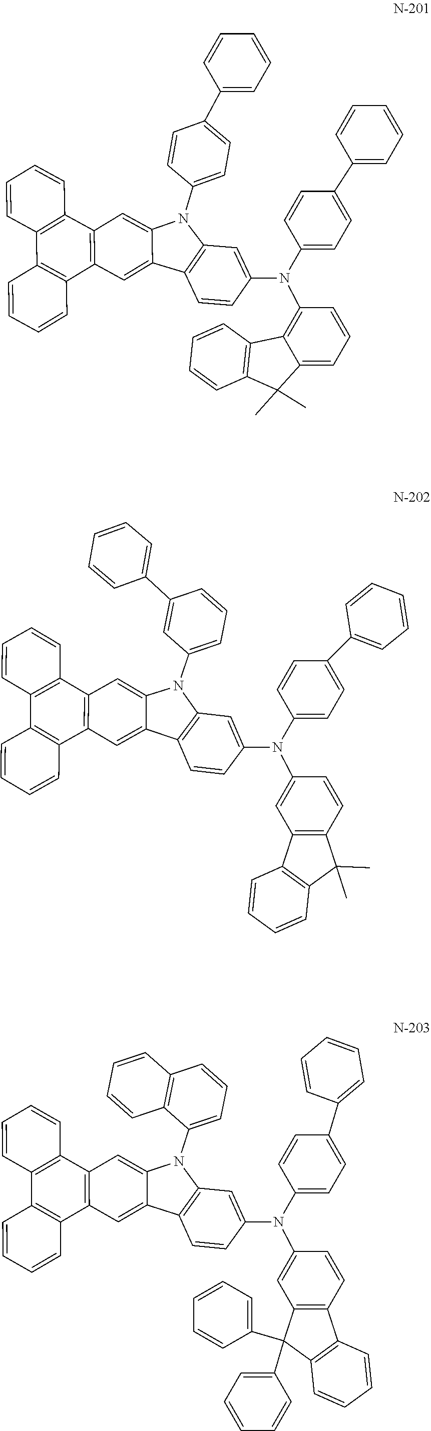 [SMILES: CC1(C)c2ccccc2-c2c(N(c3ccc(-c4ccccc4)cc3)c3ccc4c5cc6c7ccccc7c7ccccc7c6cc5n(-c5ccc(-c6ccccc6)cc5)c4c3)cccc21 ; CC1(C)c2ccccc2-c2cc(N(c3ccc(-c4ccccc4)cc3)c3ccc4c5cc6c7ccccc7c7ccccc7c6cc5n(-c5cccc(-c6ccccc6)c5)c4c3)ccc21 ; c1ccc(-c2ccc(N(c3ccc4c(c3)C(c3ccccc3)(c3ccccc3)c3ccccc3-4)c3ccc4c5cc6c7ccccc7c7ccccc7c6cc5n(-c5cccc6ccccc56)c4c3)cc2)cc1]

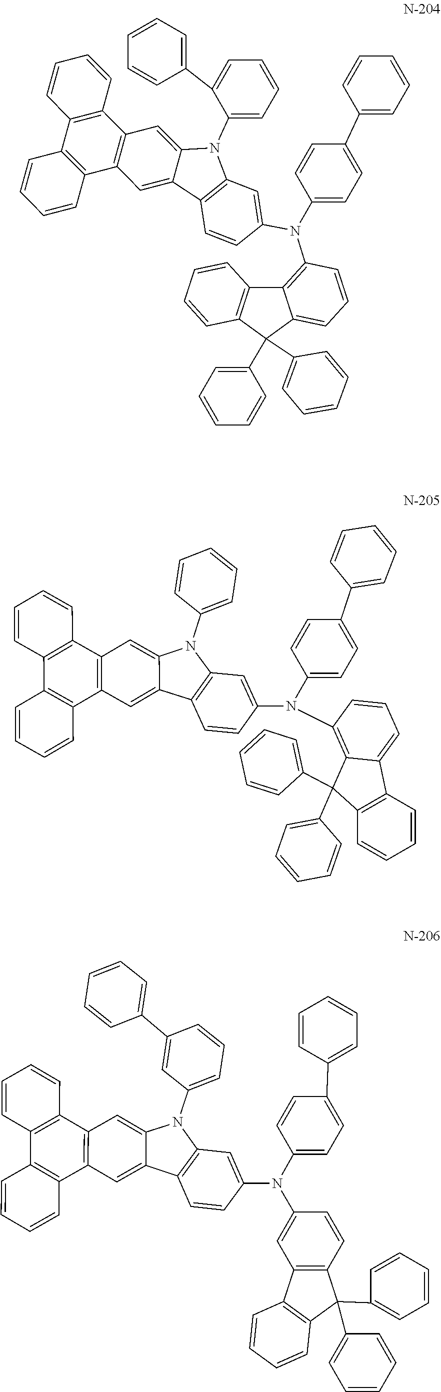 [SMILES: c1ccc(-c2ccc(N(c3ccc4c5cc6c7ccccc7c7ccccc7c6cc5n(-c5ccccc5-c5ccccc5)c4c3)c3cccc4c3-c3ccccc3C4(c3ccccc3)c3ccccc3)cc2)cc1 ; c1ccc(-c2ccc(N(c3ccc4c5cc6c7ccccc7c7ccccc7c6cc5n(-c5ccccc5)c4c3)c3cccc4c3C(c3ccccc3)(c3ccccc3)c3ccccc3-4)cc2)cc1 ; c1ccc(-c2ccc(N(c3ccc4c(c3)-c3ccccc3C4(c3ccccc3)c3ccccc3)c3ccc4c5cc6c7ccccc7c7ccccc7c6cc5n(-c5cccc(-c6ccccc6)c5)c4c3)cc2)cc1]

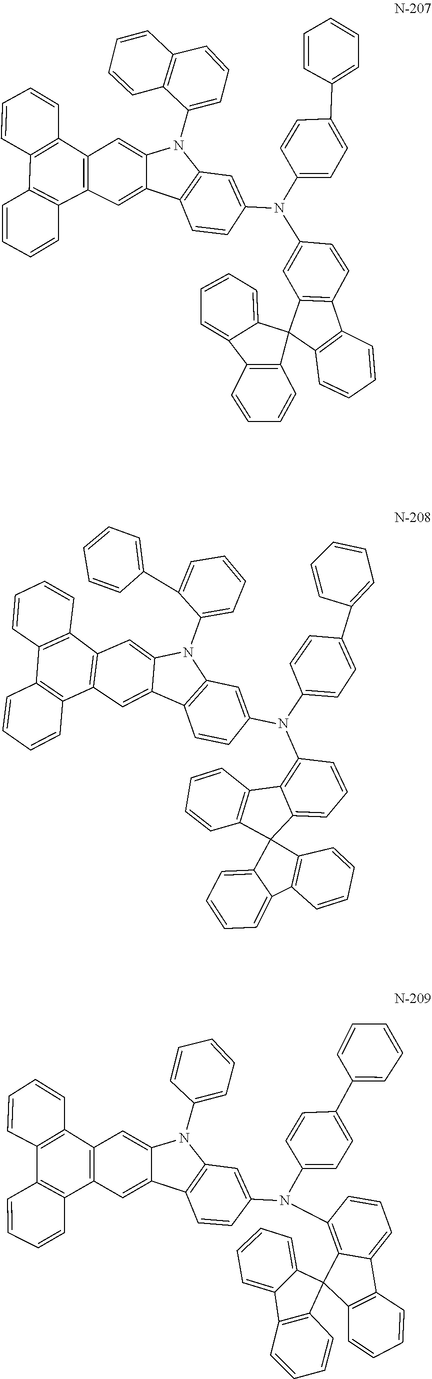 [SMILES: c1ccc(-c2ccc(N(c3ccc4c(c3)C3(c5ccccc5-c5ccccc53)c3ccccc3-4)c3ccc4c5cc6c7ccccc7c7ccccc7c6cc5n(-c5cccc6ccccc56)c4c3)cc2)cc1 ; c1ccc(-c2ccc(N(c3ccc4c5cc6c7ccccc7c7ccccc7c6cc5n(-c5ccccc5-c5ccccc5)c4c3)c3cccc4c3-c3ccccc3C43c4ccccc4-c4ccccc43)cc2)cc1 ; c1ccc(-c2ccc(N(c3ccc4c5cc6c7ccccc7c7ccccc7c6cc5n(-c5ccccc5)c4c3)c3cccc4c3C3(c5ccccc5-c5ccccc53)c3ccccc3-4)cc2)cc1]

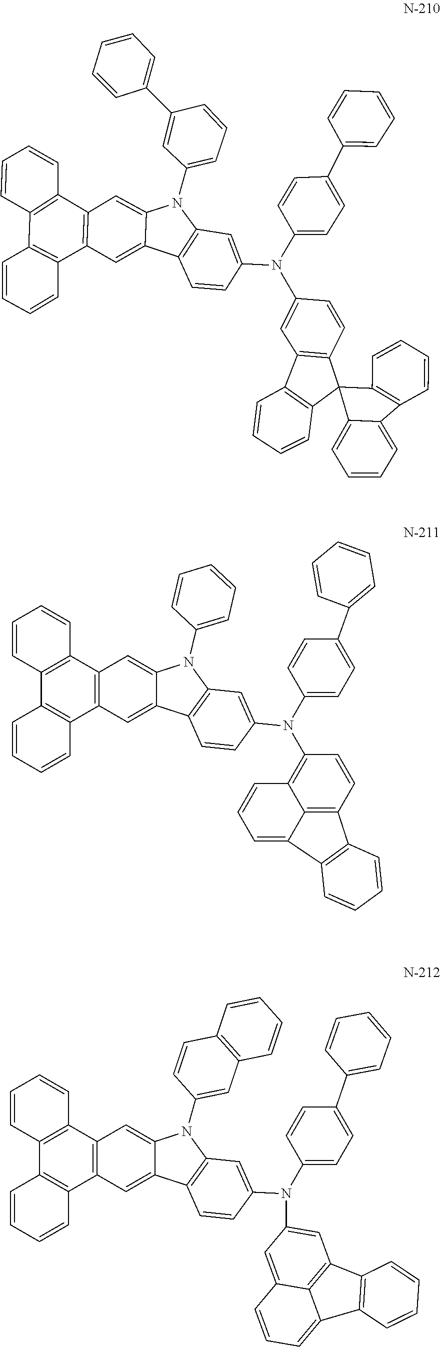 [SMILES: c1ccc(-c2ccc(N(c3ccc4c(c3)-c3ccccc3C43c4ccccc4-c4ccccc43)c3ccc4c5cc6c7ccccc7c7ccccc7c6cc5n(-c5cccc(-c6ccccc6)c5)c4c3)cc2)cc1 ; c1ccc(-c2ccc(N(c3ccc4c5cc6c7ccccc7c7ccccc7c6cc5n(-c5ccccc5)c4c3)c3ccc4c5c(cccc35)-c3ccccc3-4)cc2)cc1 ; c1ccc(-c2ccc(N(c3cc4c5c(cccc5c3)-c3ccccc3-4)c3ccc4c5cc6c7ccccc7c7ccccc7c6cc5n(-c5ccc6ccccc6c5)c4c3)cc2)cc1]

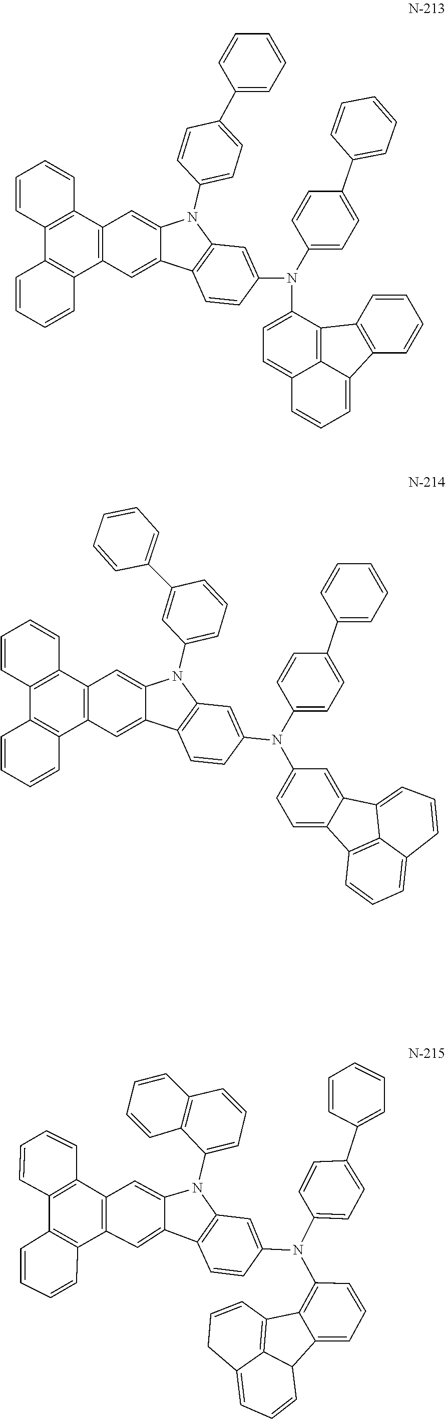 [SMILES: c1ccc(-c2ccc(N(c3ccc4c5cc6c7ccccc7c7ccccc7c6cc5n(-c5ccc(-c6ccccc6)cc5)c4c3)c3ccc4cccc5c4c3-c3ccccc3-5)cc2)cc1 ; c1ccc(-c2ccc(N(c3ccc4c(c3)-c3cccc5cccc-4c35)c3ccc4c5cc6c7ccccc7c7ccccc7c6cc5n(-c5cccc(-c6ccccc6)c5)c4c3)cc2)cc1 ; C1=CC2C3=C(C=CCC3=C1)c1c2cccc1N(c1ccc(-c2ccccc2)cc1)c1ccc2c3cc4c5ccccc5c5ccccc5c4cc3n(-c3cccc4ccccc34)c2c1]

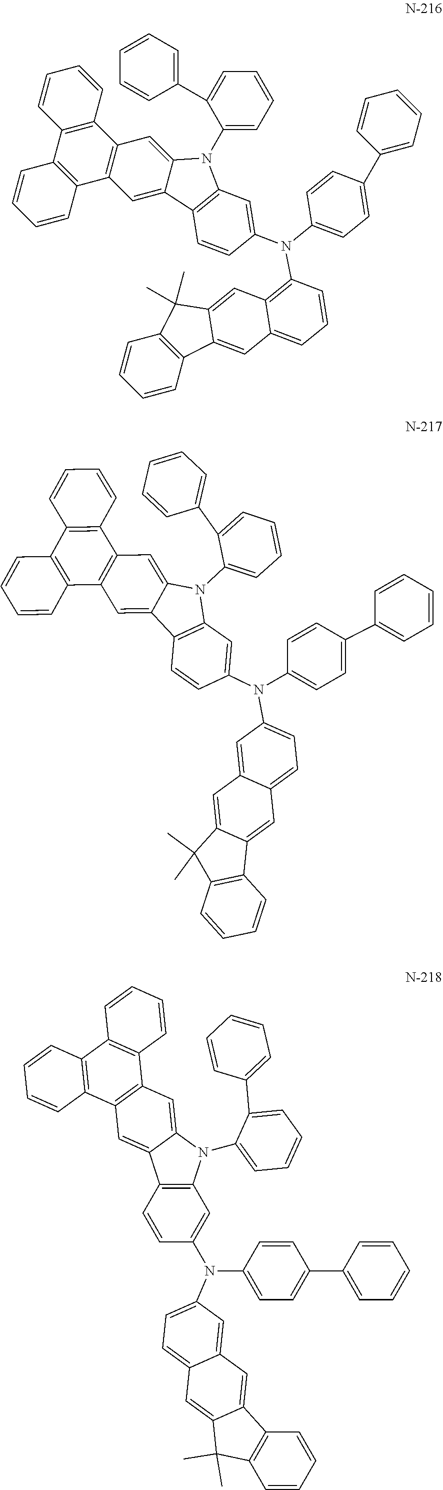 [SMILES: CC1(C)c2ccccc2-c2cc3cccc(N(c4ccc(-c5ccccc5)cc4)c4ccc5c6cc7c8ccccc8c8ccccc8c7cc6n(-c6ccccc6-c6ccccc6)c5c4)c3cc21 ; CC1(C)c2ccccc2-c2cc3ccc(N(c4ccc(-c5ccccc5)cc4)c4ccc5c6cc7c8ccccc8c8ccccc8c7cc6n(-c6ccccc6-c6ccccc6)c5c4)cc3cc21 ; CC1(C)c2ccccc2-c2cc3cc(N(c4ccc(-c5ccccc5)cc4)c4ccc5c6cc7c8ccccc8c8ccccc8c7cc6n(-c6ccccc6-c6ccccc6)c5c4)ccc3cc21]

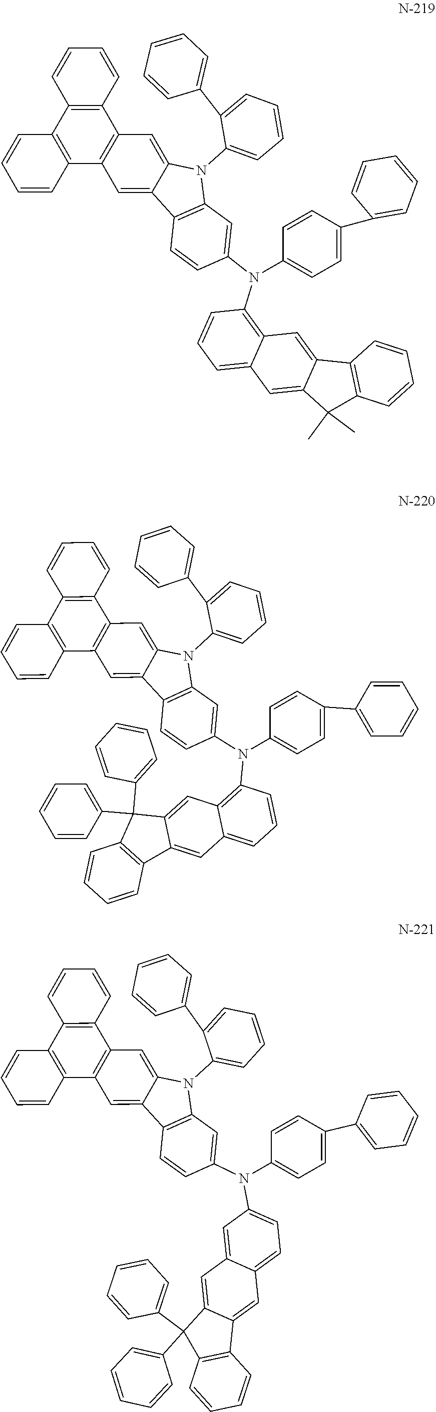 [SMILES: CC1(C)c2ccccc2-c2cc3c(N(c4ccc(-c5ccccc5)cc4)c4ccc5c6cc7c8ccccc8c8ccccc8c7cc6n(-c6ccccc6-c6ccccc6)c5c4)cccc3cc21 ; c1ccc(-c2ccc(N(c3ccc4c5cc6c7ccccc7c7ccccc7c6cc5n(-c5ccccc5-c5ccccc5)c4c3)c3cccc4cc5c(cc34)C(c3ccccc3)(c3ccccc3)c3ccccc3-5)cc2)cc1 ; c1ccc(-c2ccc(N(c3ccc4cc5c(cc4c3)C(c3ccccc3)(c3ccccc3)c3ccccc3-5)c3ccc4c5cc6c7ccccc7c7ccccc7c6cc5n(-c5ccccc5-c5ccccc5)c4c3)cc2)cc1]

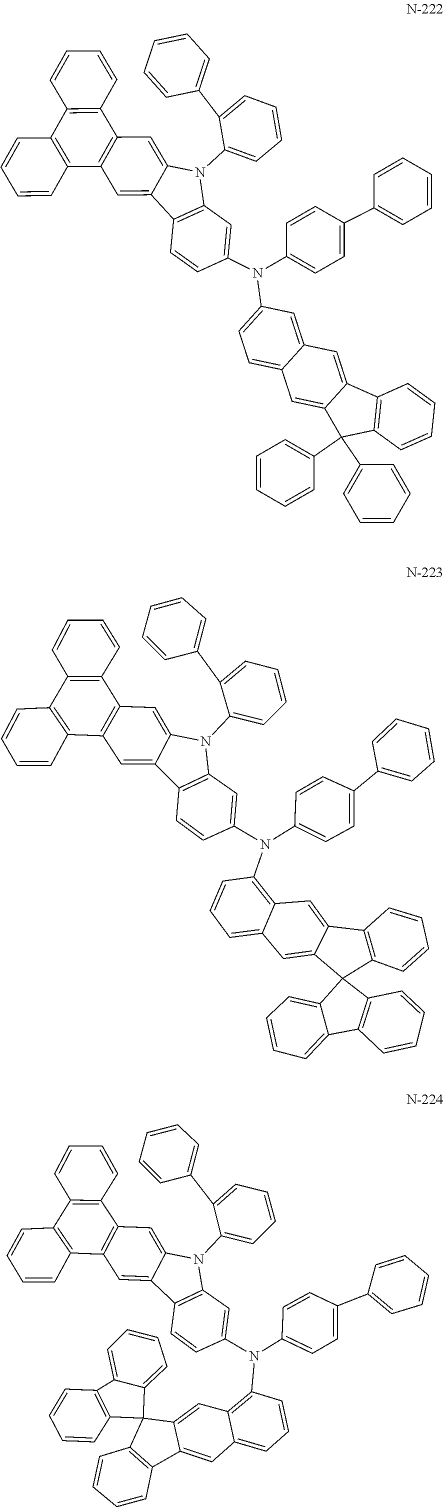 [SMILES: c1ccc(-c2ccc(N(c3ccc4cc5c(cc4c3)-c3ccccc3C5(c3ccccc3)c3ccccc3)c3ccc4c5cc6c7ccccc7c7ccccc7c6cc5n(-c5ccccc5-c5ccccc5)c4c3)cc2)cc1 ; c1ccc(-c2ccc(N(c3ccc4c5cc6c7ccccc7c7ccccc7c6cc5n(-c5ccccc5-c5ccccc5)c4c3)c3cccc4cc5c(cc34)-c3ccccc3C53c4ccccc4-c4ccccc43)cc2)cc1 ; c1ccc(-c2ccc(N(c3ccc4c5cc6c7ccccc7c7ccccc7c6cc5n(-c5ccccc5-c5ccccc5)c4c3)c3cccc4cc5c(cc34)C3(c4ccccc4-c4ccccc43)c3ccccc3-5)cc2)cc1]

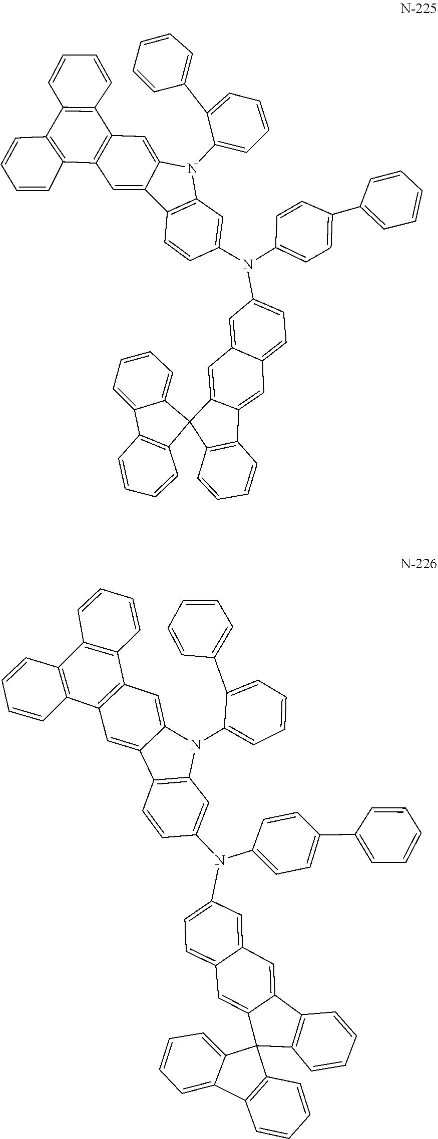 [SMILES: c1ccc(-c2ccc(N(c3ccc4cc5c(cc4c3)C3(c4ccccc4-c4ccccc43)c3ccccc3-5)c3ccc4c5cc6c7ccccc7c7ccccc7c6cc5n(-c5ccccc5-c5ccccc5)c4c3)cc2)cc1 ; c1ccc(-c2ccc(N(c3ccc4cc5c(cc4c3)-c3ccccc3C53c4ccccc4-c4ccccc43)c3ccc4c5cc6c7ccccc7c7ccccc7c6cc5n(-c5ccccc5-c5ccccc5)c4c3)cc2)cc1]

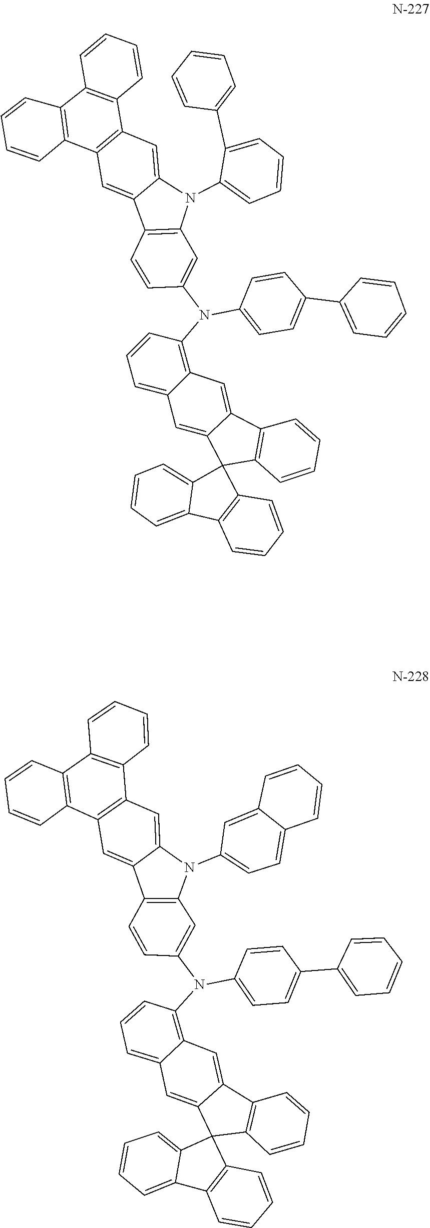 [SMILES: c1ccc(-c2ccc(N(c3ccc4c5cc6c7ccccc7c7ccccc7c6cc5n(-c5ccccc5-c5ccccc5)c4c3)c3cccc4cc5c(cc34)-c3ccccc3C53c4ccccc4-c4ccccc43)cc2)cc1 ; c1ccc(-c2ccc(N(c3ccc4c5cc6c7ccccc7c7ccccc7c6cc5n(-c5ccc6ccccc6c5)c4c3)c3cccc4cc5c(cc34)-c3ccccc3C53c4ccccc4-c4ccccc43)cc2)cc1]

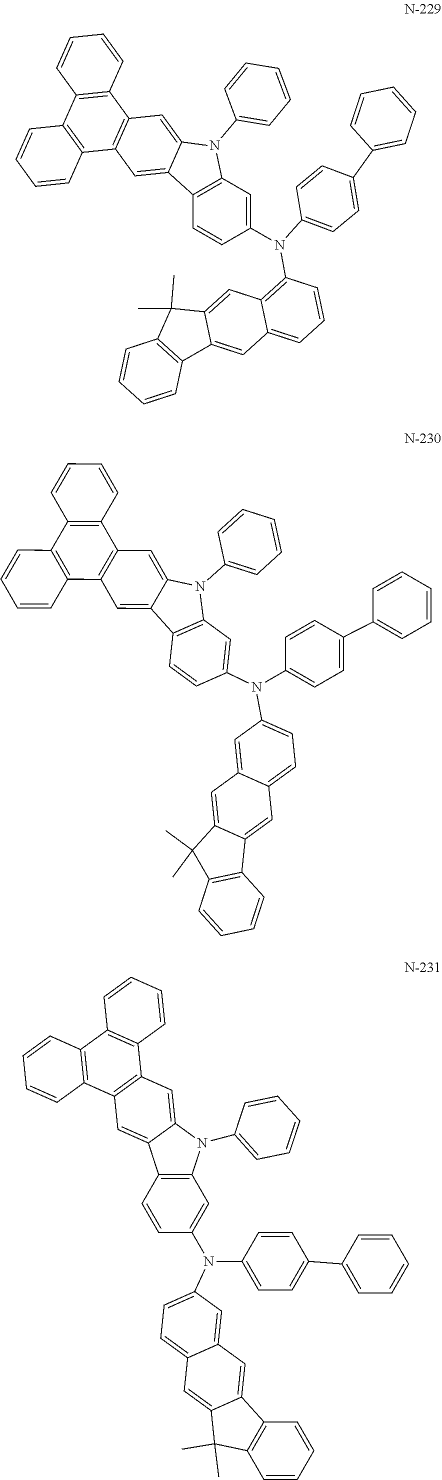 [SMILES: CC1(C)c2ccccc2-c2cc3cccc(N(c4ccc(-c5ccccc5)cc4)c4ccc5c6cc7c8ccccc8c8ccccc8c7cc6n(-c6ccccc6)c5c4)c3cc21 ; CC1(C)c2ccccc2-c2cc3ccc(N(c4ccc(-c5ccccc5)cc4)c4ccc5c6cc7c8ccccc8c8ccccc8c7cc6n(-c6ccccc6)c5c4)cc3cc21 ; CC1(C)c2ccccc2-c2cc3cc(N(c4ccc(-c5ccccc5)cc4)c4ccc5c6cc7c8ccccc8c8ccccc8c7cc6n(-c6ccccc6)c5c4)ccc3cc21]

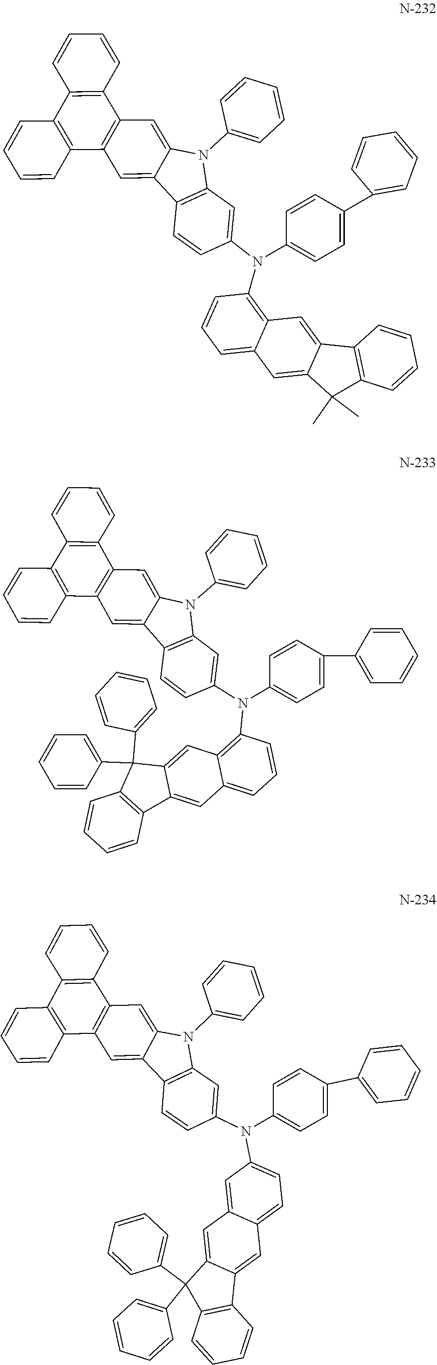 [SMILES: CC1(C)c2ccccc2-c2cc3c(N(c4ccc(-c5ccccc5)cc4)c4ccc5c6cc7c8ccccc8c8ccccc8c7cc6n(-c6ccccc6)c5c4)cccc3cc21 ; c1ccc(-c2ccc(N(c3ccc4c5cc6c7ccccc7c7ccccc7c6cc5n(-c5ccccc5)c4c3)c3cccc4cc5c(cc34)C(c3ccccc3)(c3ccccc3)c3ccccc3-5)cc2)cc1 ; c1ccc(-c2ccc(N(c3ccc4cc5c(cc4c3)C(c3ccccc3)(c3ccccc3)c3ccccc3-5)c3ccc4c5cc6c7ccccc7c7ccccc7c6cc5n(-c5ccccc5)c4c3)cc2)cc1]

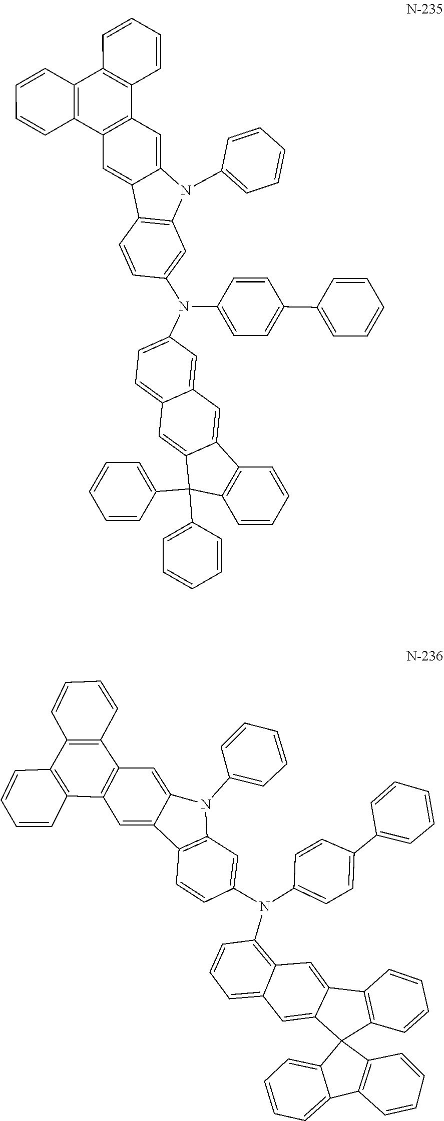 [SMILES: c1ccc(-c2ccc(N(c3ccc4cc5c(cc4c3)-c3ccccc3C5(c3ccccc3)c3ccccc3)c3ccc4c5cc6c7ccccc7c7ccccc7c6cc5n(-c5ccccc5)c4c3)cc2)cc1 ; c1ccc(-c2ccc(N(c3ccc4c5cc6c7ccccc7c7ccccc7c6cc5n(-c5ccccc5)c4c3)c3cccc4cc5c(cc34)-c3ccccc3C53c4ccccc4-c4ccccc43)cc2)cc1]

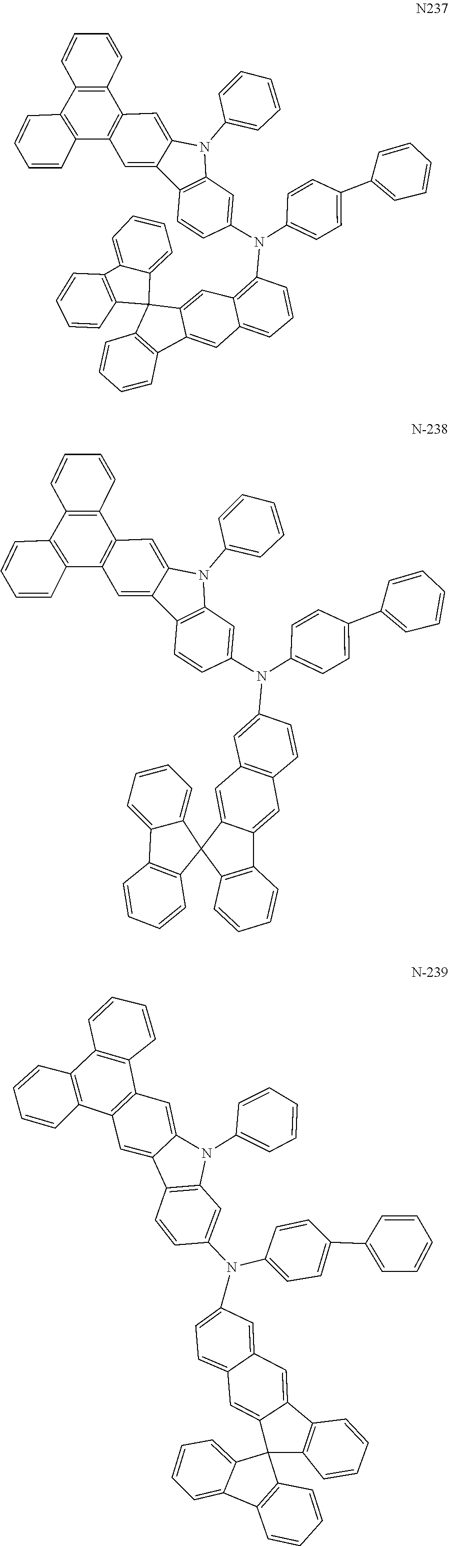 [SMILES: c1ccc(-c2ccc(N(c3ccc4c5cc6c7ccccc7c7ccccc7c6cc5n(-c5ccccc5)c4c3)c3cccc4cc5c(cc34)C3(c4ccccc4-c4ccccc43)c3ccccc3-5)cc2)cc1 ; c1ccc(-c2ccc(N(c3ccc4cc5c(cc4c3)C3(c4ccccc4-c4ccccc43)c3ccccc3-5)c3ccc4c5cc6c7ccccc7c7ccccc7c6cc5n(-c5ccccc5)c4c3)cc2)cc1 ; c1ccc(-c2ccc(N(c3ccc4cc5c(cc4c3)-c3ccccc3C53c4ccccc4-c4ccccc43)c3ccc4c5cc6c7ccccc7c7ccccc7c6cc5n(-c5ccccc5)c4c3)cc2)cc1]

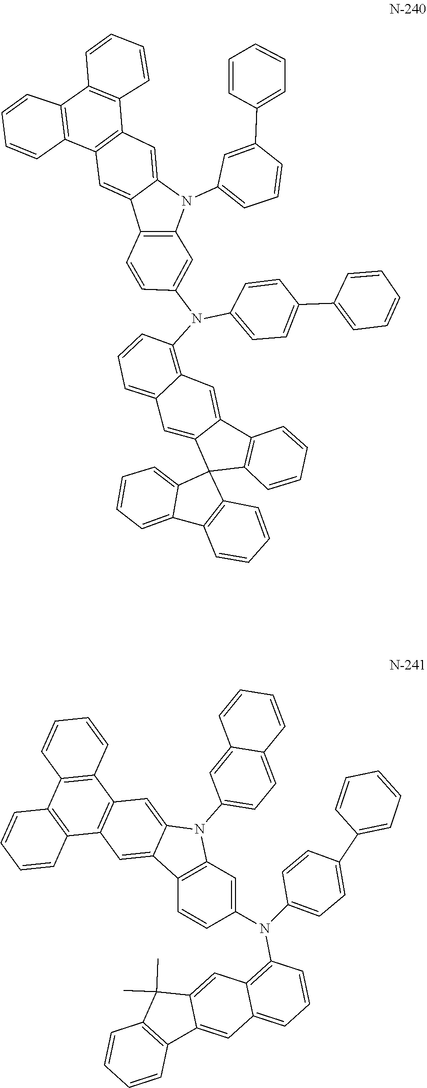 [SMILES: c1ccc(-c2ccc(N(c3ccc4c5cc6c7ccccc7c7ccccc7c6cc5n(-c5cccc(-c6ccccc6)c5)c4c3)c3cccc4cc5c(cc34)-c3ccccc3C53c4ccccc4-c4ccccc43)cc2)cc1 ; CC1(C)c2ccccc2-c2cc3cccc(N(c4ccc(-c5ccccc5)cc4)c4ccc5c6cc7c8ccccc8c8ccccc8c7cc6n(-c6ccc7ccccc7c6)c5c4)c3cc21]

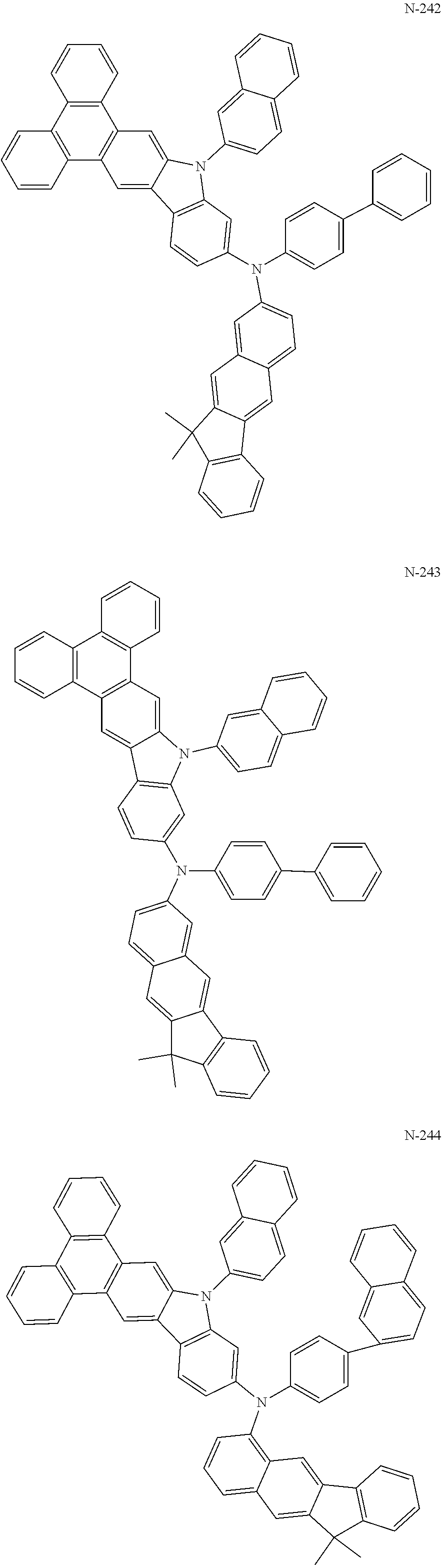 [SMILES: CC1(C)c2ccccc2-c2cc3ccc(N(c4ccc(-c5ccccc5)cc4)c4ccc5c6cc7c8ccccc8c8ccccc8c7cc6n(-c6ccc7ccccc7c6)c5c4)cc3cc21 ; CC1(C)c2ccccc2-c2cc3cc(N(c4ccc(-c5ccccc5)cc4)c4ccc5c6cc7c8ccccc8c8ccccc8c7cc6n(-c6ccc7ccccc7c6)c5c4)ccc3cc21 ; CC1(C)c2ccccc2-c2cc3c(N(c4ccc(-c5ccc6ccccc6c5)cc4)c4ccc5c6cc7c8ccccc8c8ccccc8c7cc6n(-c6ccc7ccccc7c6)c5c4)cccc3cc21]

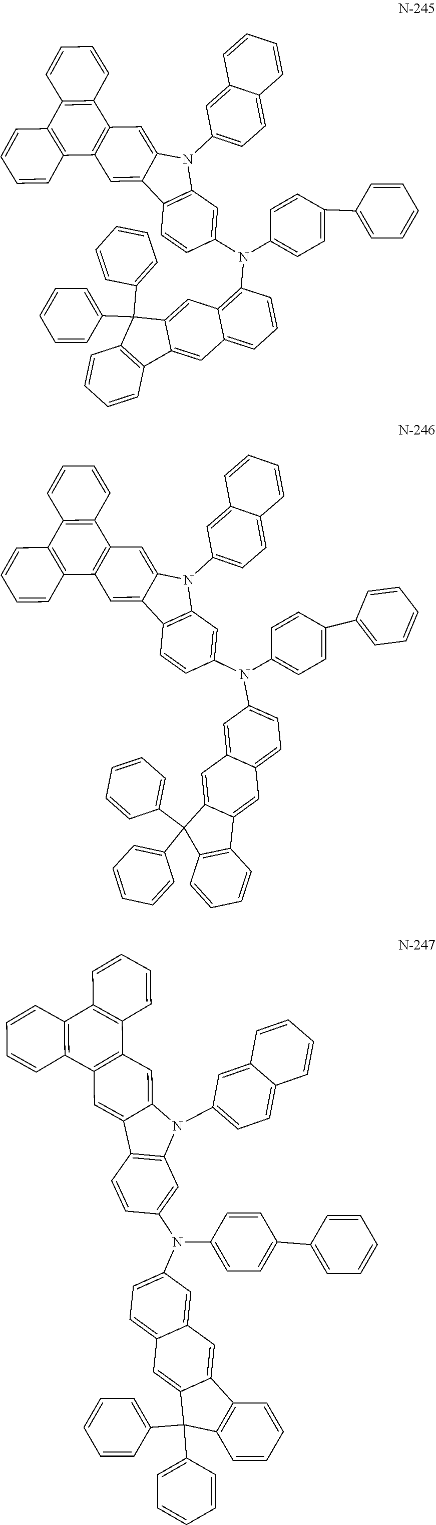 [SMILES: c1ccc(-c2ccc(N(c3ccc4c5cc6c7ccccc7c7ccccc7c6cc5n(-c5ccc6ccccc6c5)c4c3)c3cccc4cc5c(cc34)C(c3ccccc3)(c3ccccc3)c3ccccc3-5)cc2)cc1 ; c1ccc(-c2ccc(N(c3ccc4cc5c(cc4c3)C(c3ccccc3)(c3ccccc3)c3ccccc3-5)c3ccc4c5cc6c7ccccc7c7ccccc7c6cc5n(-c5ccc6ccccc6c5)c4c3)cc2)cc1 ; c1ccc(-c2ccc(N(c3ccc4cc5c(cc4c3)-c3ccccc3C5(c3ccccc3)c3ccccc3)c3ccc4c5cc6c7ccccc7c7ccccc7c6cc5n(-c5ccc6ccccc6c5)c4c3)cc2)cc1]

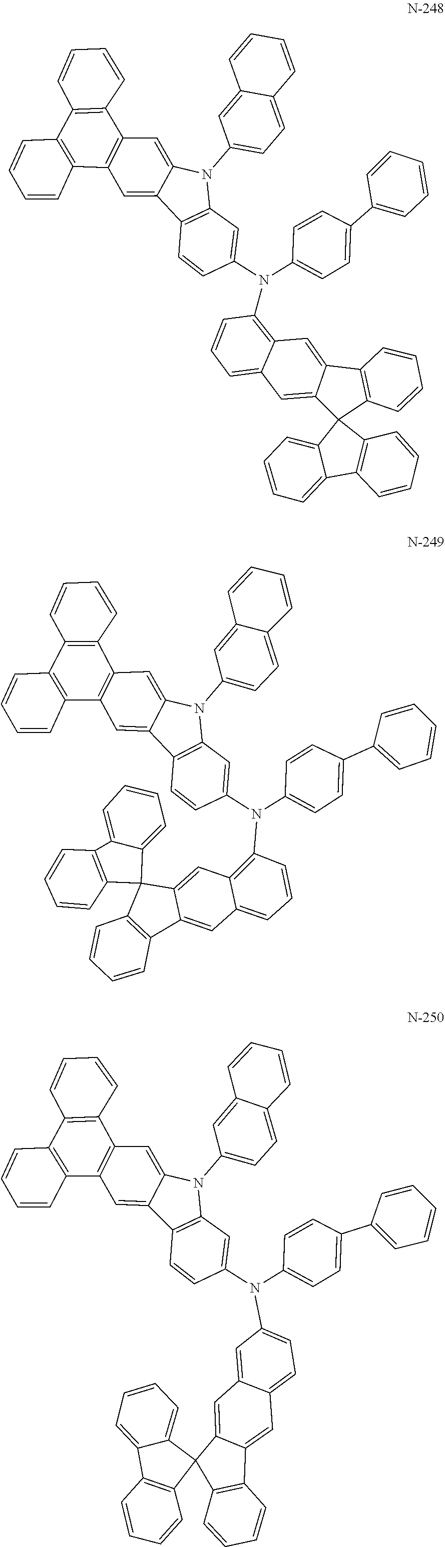 [SMILES: c1ccc(-c2ccc(N(c3ccc4c5cc6c7ccccc7c7ccccc7c6cc5n(-c5ccc6ccccc6c5)c4c3)c3cccc4cc5c(cc34)-c3ccccc3C53c4ccccc4-c4ccccc43)cc2)cc1 ; c1ccc(-c2ccc(N(c3ccc4c5cc6c7ccccc7c7ccccc7c6cc5n(-c5ccc6ccccc6c5)c4c3)c3cccc4cc5c(cc34)C3(c4ccccc4-c4ccccc43)c3ccccc3-5)cc2)cc1 ; c1ccc(-c2ccc(N(c3ccc4cc5c(cc4c3)C3(c4ccccc4-c4ccccc43)c3ccccc3-5)c3ccc4c5cc6c7ccccc7c7ccccc7c6cc5n(-c5ccc6ccccc6c5)c4c3)cc2)cc1]

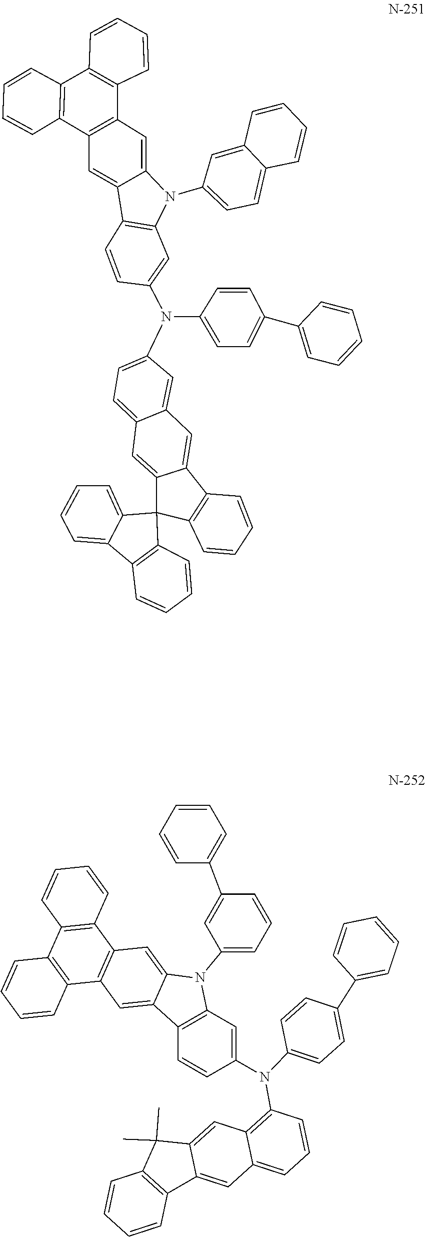 [SMILES: c1ccc(-c2ccc(N(c3ccc4cc5c(cc4c3)-c3ccccc3C53c4ccccc4-c4ccccc43)c3ccc4c5cc6c7ccccc7c7ccccc7c6cc5n(-c5ccc6ccccc6c5)c4c3)cc2)cc1 ; CC1(C)c2ccccc2-c2cc3cccc(N(c4ccc(-c5ccccc5)cc4)c4ccc5c6cc7c8ccccc8c8ccccc8c7cc6n(-c6cccc(-c7ccccc7)c6)c5c4)c3cc21]

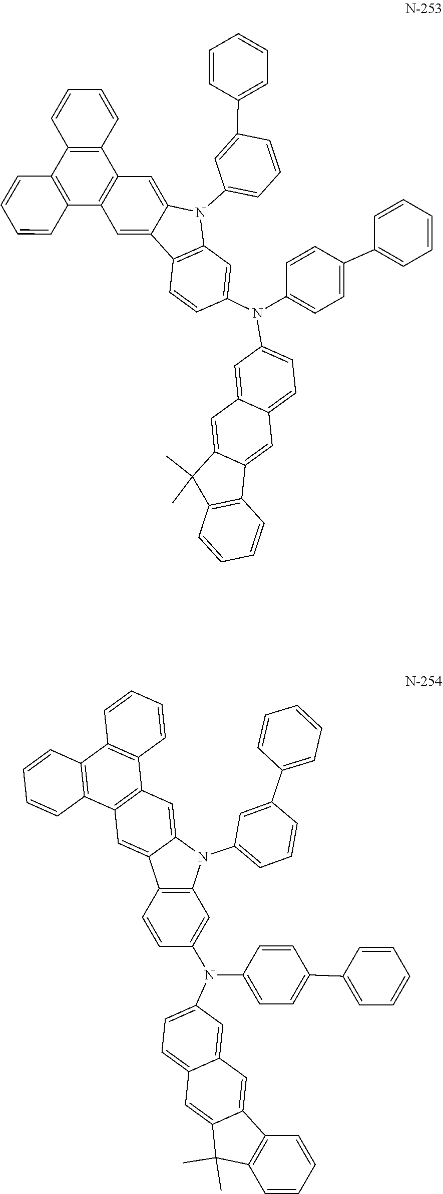 [SMILES: CC1(C)c2ccccc2-c2cc3ccc(N(c4ccc(-c5ccccc5)cc4)c4ccc5c6cc7c8ccccc8c8ccccc8c7cc6n(-c6cccc(-c7ccccc7)c6)c5c4)cc3cc21 ; CC1(C)c2ccccc2-c2cc3cc(N(c4ccc(-c5ccccc5)cc4)c4ccc5c6cc7c8ccccc8c8ccccc8c7cc6n(-c6cccc(-c7ccccc7)c6)c5c4)ccc3cc21]

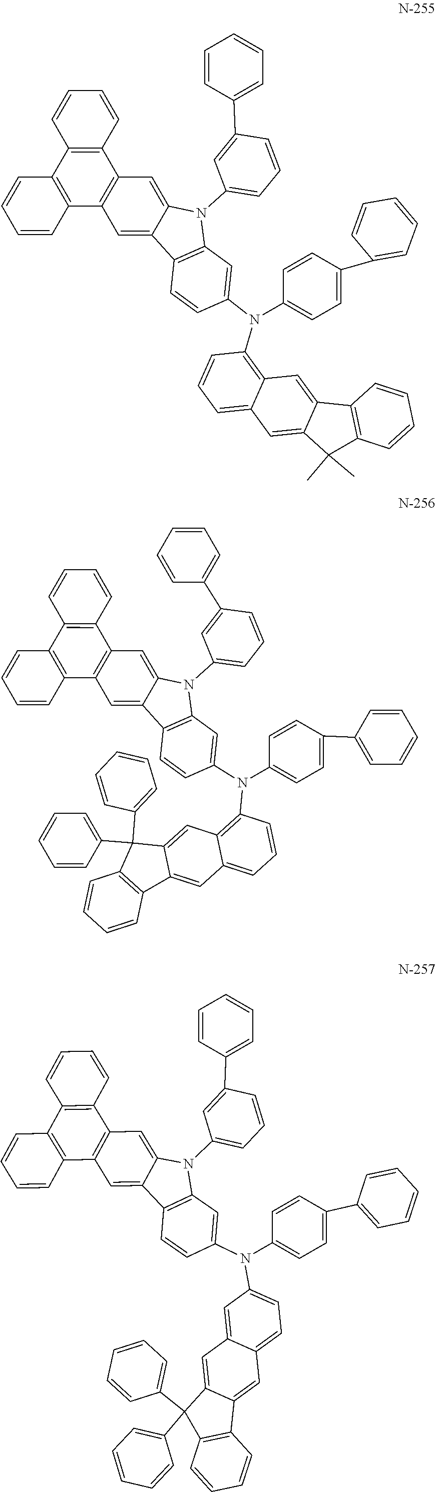 [SMILES: CC1(C)c2ccccc2-c2cc3c(N(c4ccc(-c5ccccc5)cc4)c4ccc5c6cc7c8ccccc8c8ccccc8c7cc6n(-c6cccc(-c7ccccc7)c6)c5c4)cccc3cc21 ; c1ccc(-c2ccc(N(c3ccc4c5cc6c7ccccc7c7ccccc7c6cc5n(-c5cccc(-c6ccccc6)c5)c4c3)c3cccc4cc5c(cc34)C(c3ccccc3)(c3ccccc3)c3ccccc3-5)cc2)cc1 ; c1ccc(-c2ccc(N(c3ccc4cc5c(cc4c3)C(c3ccccc3)(c3ccccc3)c3ccccc3-5)c3ccc4c5cc6c7ccccc7c7ccccc7c6cc5n(-c5cccc(-c6ccccc6)c5)c4c3)cc2)cc1]

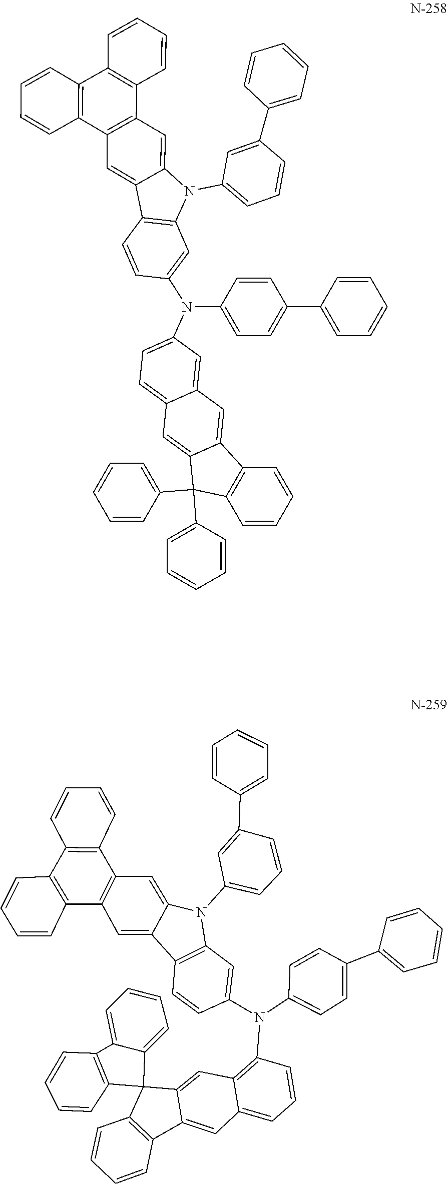 [SMILES: c1ccc(-c2ccc(N(c3ccc4cc5c(cc4c3)-c3ccccc3C5(c3ccccc3)c3ccccc3)c3ccc4c5cc6c7ccccc7c7ccccc7c6cc5n(-c5cccc(-c6ccccc6)c5)c4c3)cc2)cc1 ; c1ccc(-c2ccc(N(c3ccc4c5cc6c7ccccc7c7ccccc7c6cc5n(-c5cccc(-c6ccccc6)c5)c4c3)c3cccc4cc5c(cc34)C3(c4ccccc4-c4ccccc43)c3ccccc3-5)cc2)cc1]

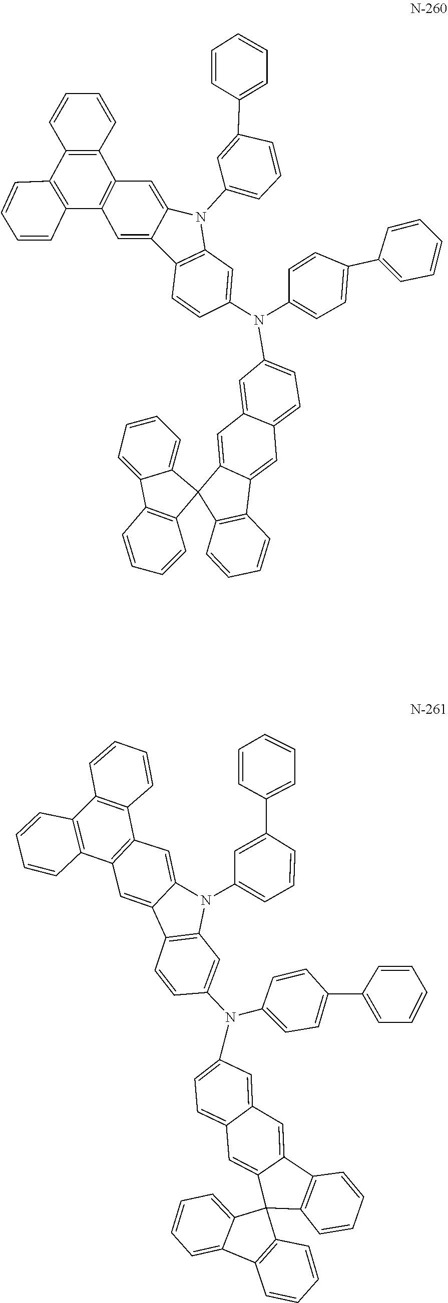 [SMILES: c1ccc(-c2ccc(N(c3ccc4cc5c(cc4c3)C3(c4ccccc4-c4ccccc43)c3ccccc3-5)c3ccc4c5cc6c7ccccc7c7ccccc7c6cc5n(-c5cccc(-c6ccccc6)c5)c4c3)cc2)cc1 ; c1ccc(-c2ccc(N(c3ccc4cc5c(cc4c3)-c3ccccc3C53c4ccccc4-c4ccccc43)c3ccc4c5cc6c7ccccc7c7ccccc7c6cc5n(-c5cccc(-c6ccccc6)c5)c4c3)cc2)cc1]

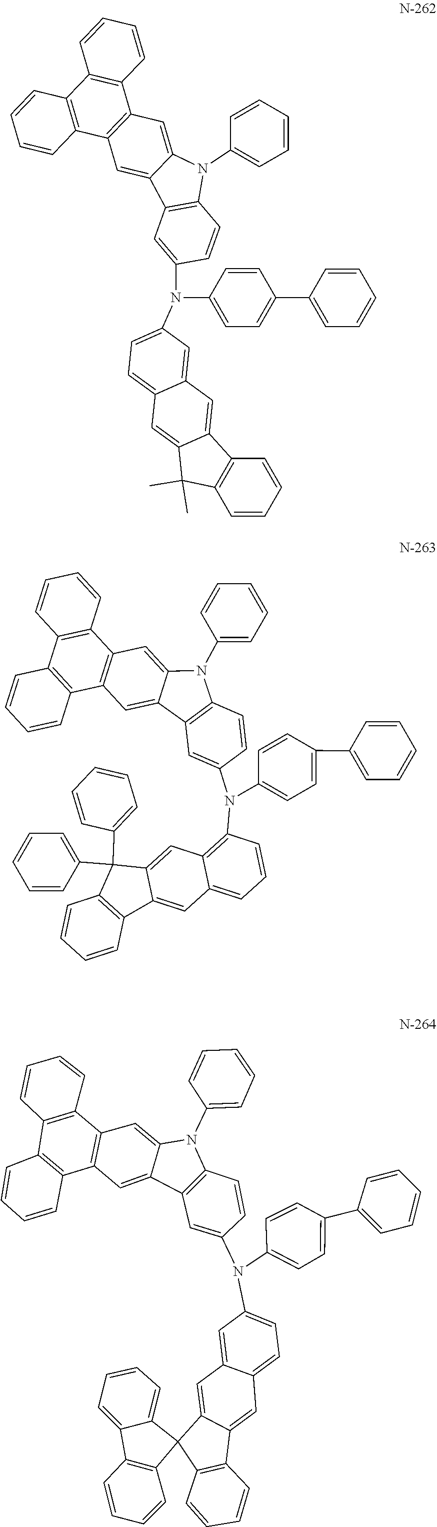 [SMILES: CC1(C)c2ccccc2-c2cc3cc(N(c4ccc(-c5ccccc5)cc4)c4ccc5c(c4)c4cc6c7ccccc7c7ccccc7c6cc4n5-c4ccccc4)ccc3cc21 ; c1ccc(-c2ccc(N(c3ccc4c(c3)c3cc5c6ccccc6c6ccccc6c5cc3n4-c3ccccc3)c3cccc4cc5c(cc34)C(c3ccccc3)(c3ccccc3)c3ccccc3-5)cc2)cc1 ; c1ccc(-c2ccc(N(c3ccc4cc5c(cc4c3)C3(c4ccccc4-c4ccccc43)c3ccccc3-5)c3ccc4c(c3)c3cc5c6ccccc6c6ccccc6c5cc3n4-c3ccccc3)cc2)cc1]

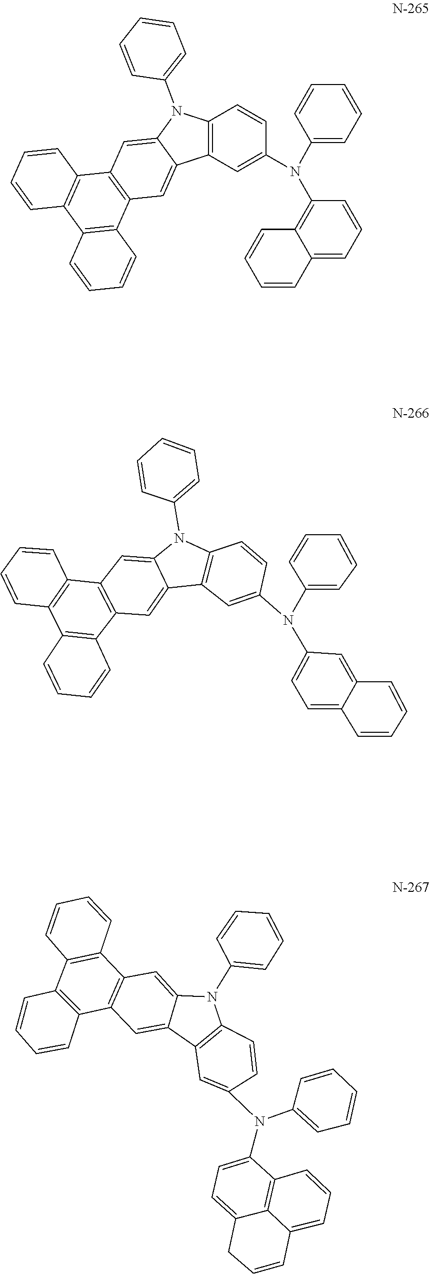 [SMILES: c1ccc(N(c2ccc3c(c2)c2cc4c5ccccc5c5ccccc5c4cc2n3-c2ccccc2)c2cccc3ccccc23)cc1 ; c1ccc(N(c2ccc3ccccc3c2)c2ccc3c(c2)c2cc4c5ccccc5c5ccccc5c4cc2n3-c2ccccc2)cc1 ; C1=Cc2cccc3c(N(c4ccccc4)c4ccc5c(c4)c4cc6c7ccccc7c7ccccc7c6cc4n5-c4ccccc4)ccc(c23)C1]

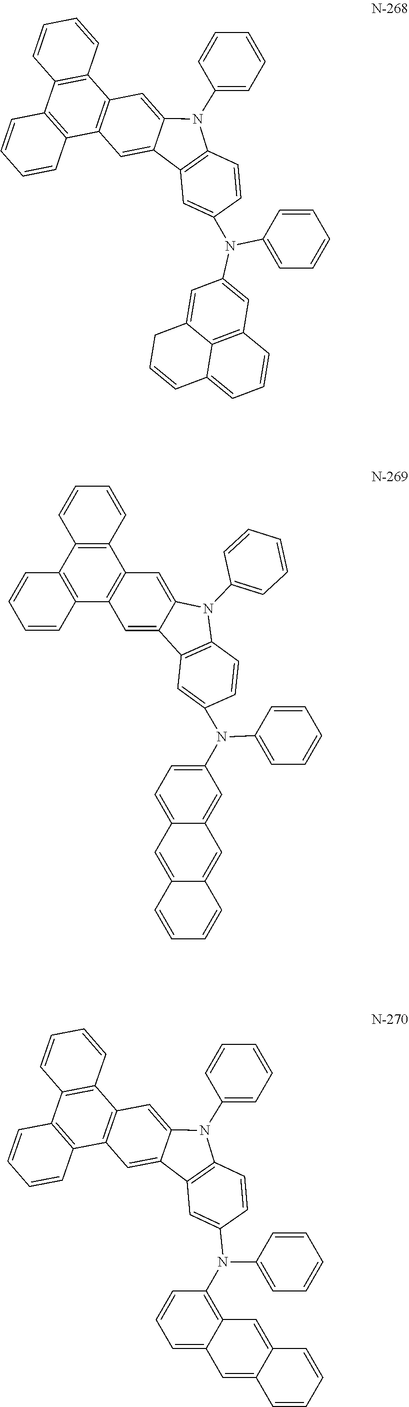 [SMILES: C1=Cc2cccc3cc(N(c4ccccc4)c4ccc5c(c4)c4cc6c7ccccc7c7ccccc7c6cc4n5-c4ccccc4)cc(c23)C1 ; c1ccc(N(c2ccc3cc4ccccc4cc3c2)c2ccc3c(c2)c2cc4c5ccccc5c5ccccc5c4cc2n3-c2ccccc2)cc1 ; c1ccc(N(c2ccc3c(c2)c2cc4c5ccccc5c5ccccc5c4cc2n3-c2ccccc2)c2cccc3cc4ccccc4cc23)cc1]

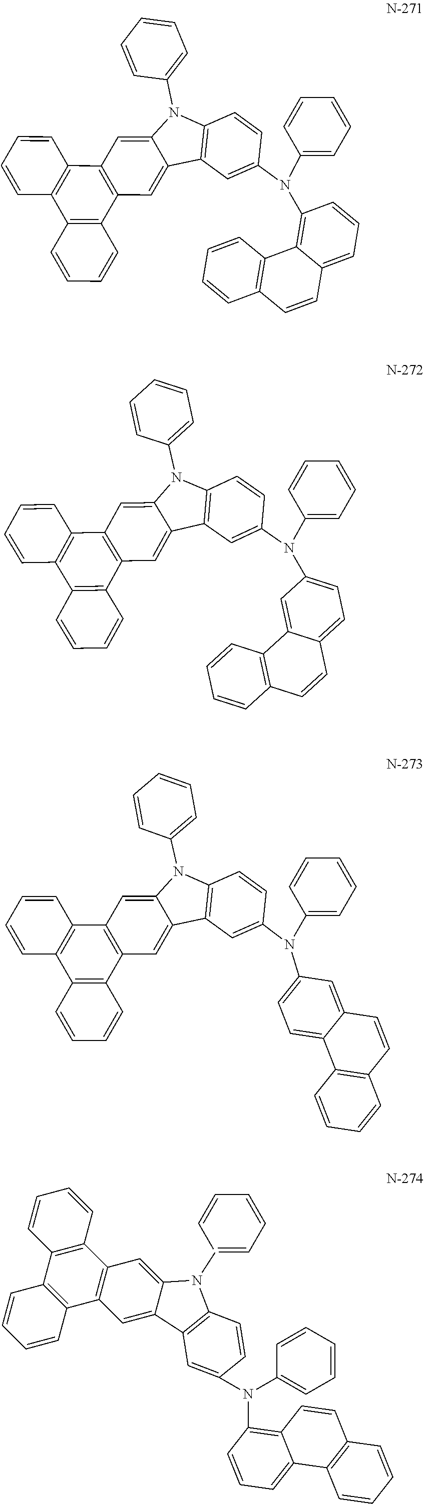

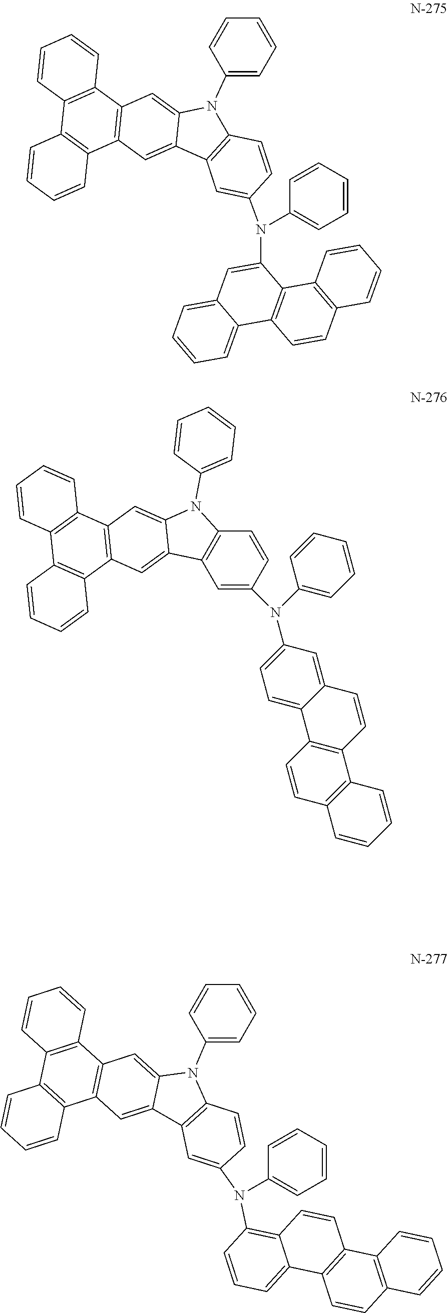

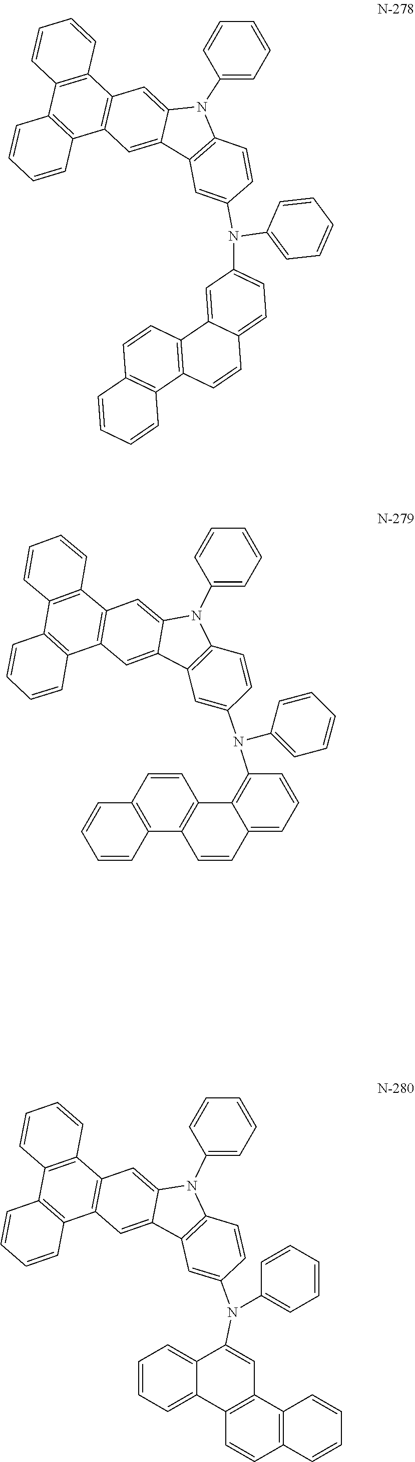 [SMILES: c1ccc(N(c2ccc3ccc4c5ccccc5ccc4c3c2)c2ccc3c(c2)c2cc4c5ccccc5c5ccccc5c4cc2n3-c2ccccc2)cc1 ; c1ccc(N(c2ccc3c(c2)c2cc4c5ccccc5c5ccccc5c4cc2n3-c2ccccc2)c2cccc3ccc4c5ccccc5ccc4c23)cc1 ; c1ccc(N(c2ccc3c(c2)c2cc4c5ccccc5c5ccccc5c4cc2n3-c2ccccc2)c2cc3c4ccccc4ccc3c3ccccc23)cc1]

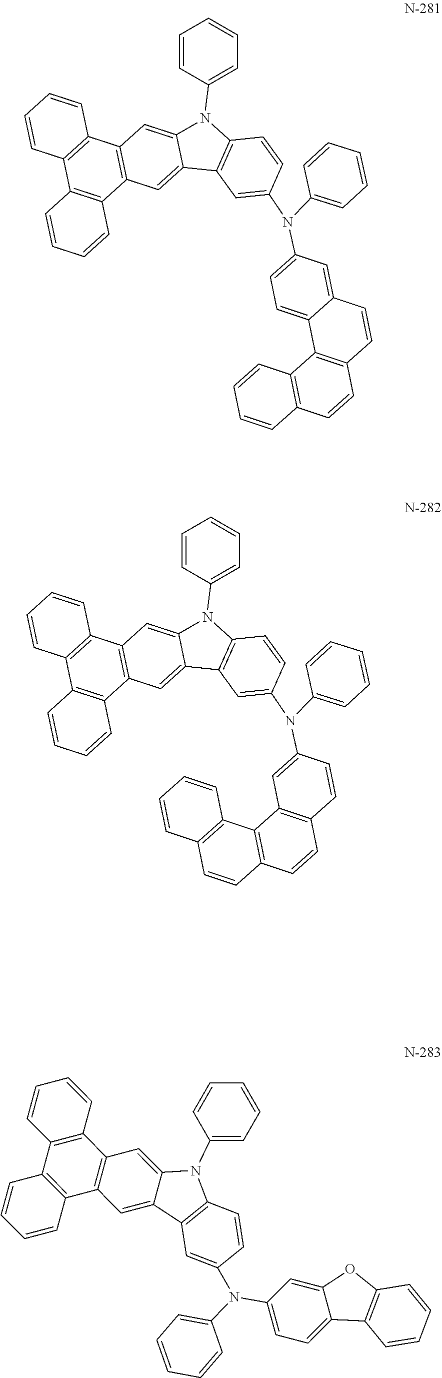 [SMILES: c1ccc(N(c2ccc3c(ccc4ccc5ccccc5c43)c2)c2ccc3c(c2)c2cc4c5ccccc5c5ccccc5c4cc2n3-c2ccccc2)cc1 ; c1ccc(N(c2ccc3ccc4ccc5ccccc5c4c3c2)c2ccc3c(c2)c2cc4c5ccccc5c5ccccc5c4cc2n3-c2ccccc2)cc1 ; c1ccc(N(c2ccc3c(c2)oc2ccccc23)c2ccc3c(c2)c2cc4c5ccccc5c5ccccc5c4cc2n3-c2ccccc2)cc1]

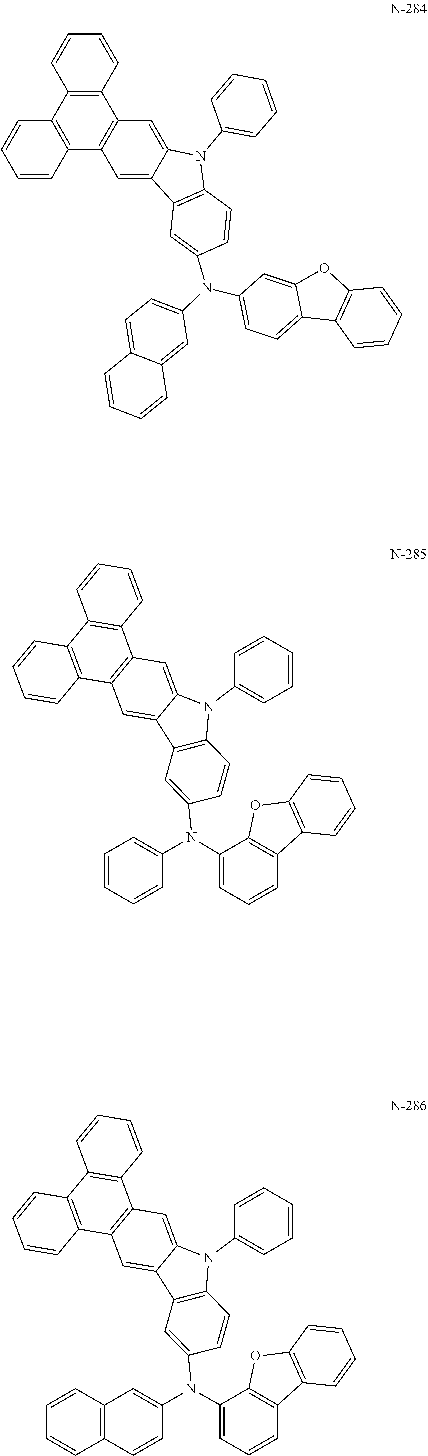 [SMILES: c1ccc(-n2c3ccc(N(c4ccc5ccccc5c4)c4ccc5c(c4)oc4ccccc45)cc3c3cc4c5ccccc5c5ccccc5c4cc32)cc1 ; c1ccc(N(c2ccc3c(c2)c2cc4c5ccccc5c5ccccc5c4cc2n3-c2ccccc2)c2cccc3c2oc2ccccc23)cc1 ; c1ccc(-n2c3ccc(N(c4ccc5ccccc5c4)c4cccc5c4oc4ccccc45)cc3c3cc4c5ccccc5c5ccccc5c4cc32)cc1]

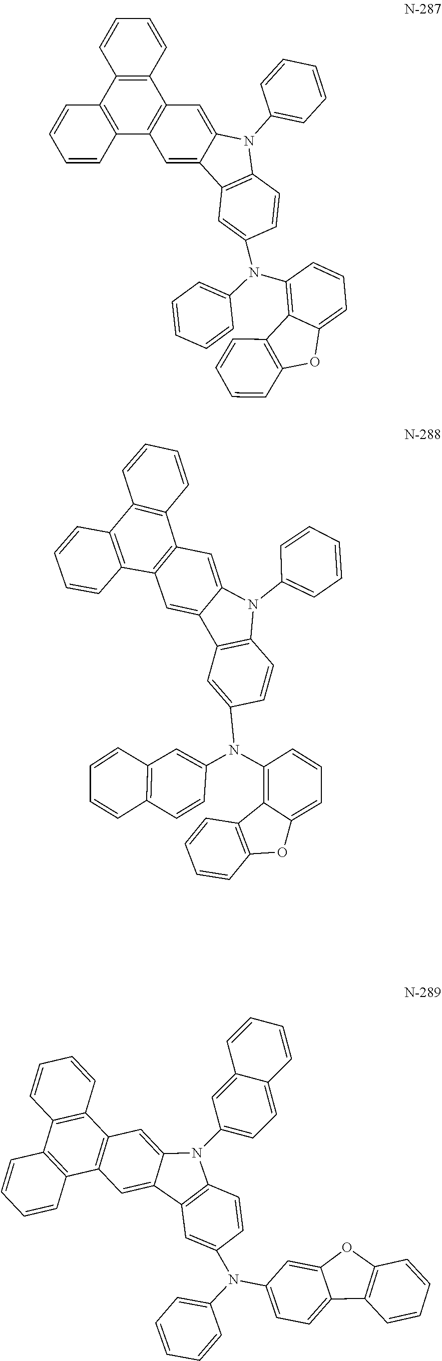 [SMILES: c1ccc(N(c2ccc3c(c2)c2cc4c5ccccc5c5ccccc5c4cc2n3-c2ccccc2)c2cccc3oc4ccccc4c23)cc1 ; c1ccc(-n2c3ccc(N(c4ccc5ccccc5c4)c4cccc5oc6ccccc6c45)cc3c3cc4c5ccccc5c5ccccc5c4cc32)cc1 ; c1ccc(N(c2ccc3c(c2)oc2ccccc23)c2ccc3c(c2)c2cc4c5ccccc5c5ccccc5c4cc2n3-c2ccc3ccccc3c2)cc1]

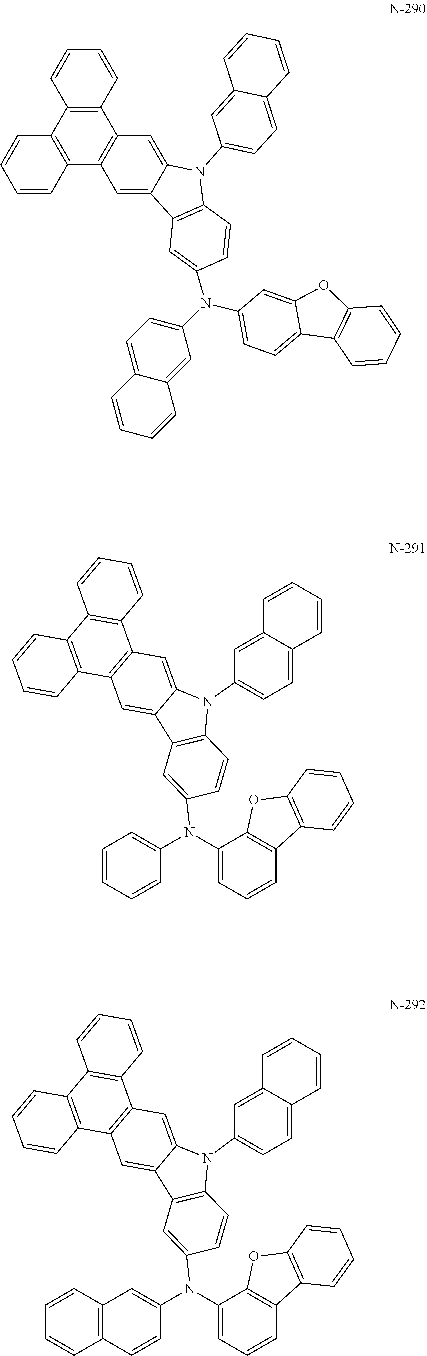 [SMILES: c1ccc2cc(N(c3ccc4c(c3)oc3ccccc34)c3ccc4c(c3)c3cc5c6ccccc6c6ccccc6c5cc3n4-c3ccc4ccccc4c3)ccc2c1 ; c1ccc(N(c2ccc3c(c2)c2cc4c5ccccc5c5ccccc5c4cc2n3-c2ccc3ccccc3c2)c2cccc3c2oc2ccccc23)cc1 ; c1ccc2cc(N(c3ccc4c(c3)c3cc5c6ccccc6c6ccccc6c5cc3n4-c3ccc4ccccc4c3)c3cccc4c3oc3ccccc34)ccc2c1]

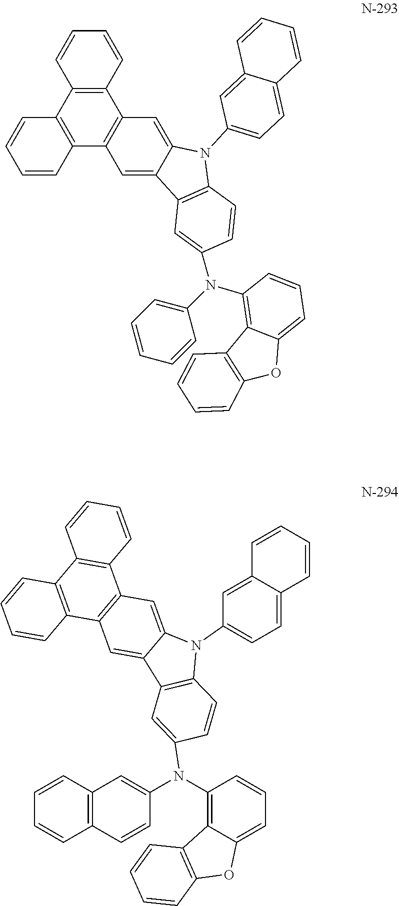 [SMILES: c1ccc(N(c2ccc3c(c2)c2cc4c5ccccc5c5ccccc5c4cc2n3-c2ccc3ccccc3c2)c2cccc3oc4ccccc4c23)cc1 ; c1ccc2cc(N(c3ccc4c(c3)c3cc5c6ccccc6c6ccccc6c5cc3n4-c3ccc4ccccc4c3)c3cccc4oc5ccccc5c34)ccc2c1]

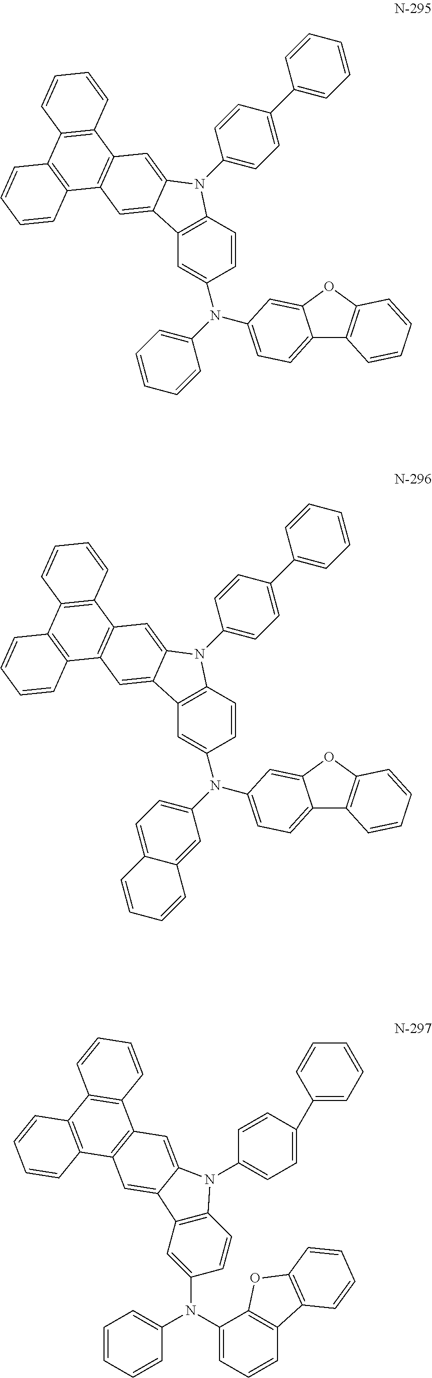 [SMILES: c1ccc(-c2ccc(-n3c4ccc(N(c5ccccc5)c5ccc6c(c5)oc5ccccc56)cc4c4cc5c6ccccc6c6ccccc6c5cc43)cc2)cc1 ; c1ccc(-c2ccc(-n3c4ccc(N(c5ccc6ccccc6c5)c5ccc6c(c5)oc5ccccc56)cc4c4cc5c6ccccc6c6ccccc6c5cc43)cc2)cc1 ; c1ccc(-c2ccc(-n3c4ccc(N(c5ccccc5)c5cccc6c5oc5ccccc56)cc4c4cc5c6ccccc6c6ccccc6c5cc43)cc2)cc1]

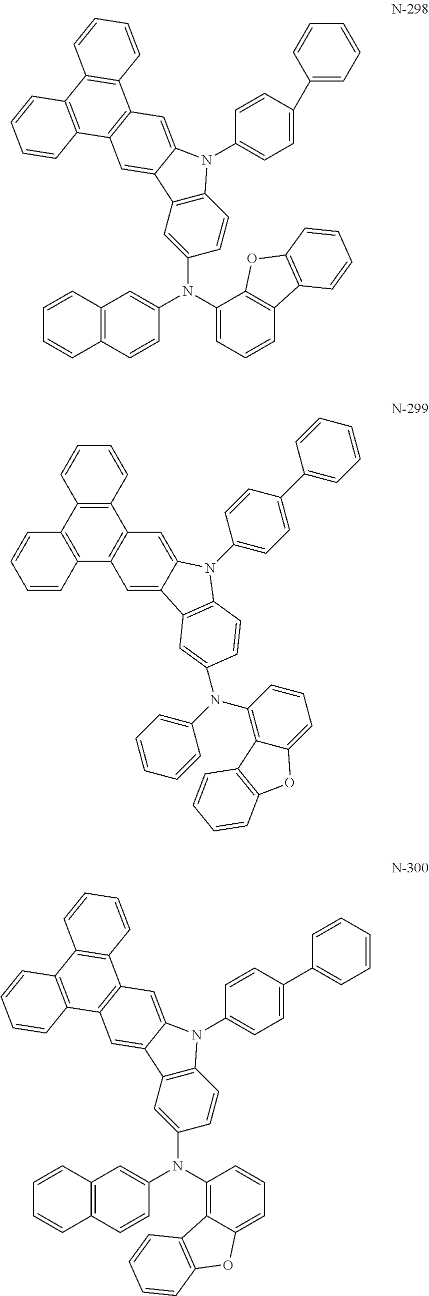 [SMILES: c1ccc(-c2ccc(-n3c4ccc(N(c5ccc6ccccc6c5)c5cccc6c5oc5ccccc56)cc4c4cc5c6ccccc6c6ccccc6c5cc43)cc2)cc1 ; c1ccc(-c2ccc(-n3c4ccc(N(c5ccccc5)c5cccc6oc7ccccc7c56)cc4c4cc5c6ccccc6c6ccccc6c5cc43)cc2)cc1 ; c1ccc(-c2ccc(-n3c4ccc(N(c5ccc6ccccc6c5)c5cccc6oc7ccccc7c56)cc4c4cc5c6ccccc6c6ccccc6c5cc43)cc2)cc1]

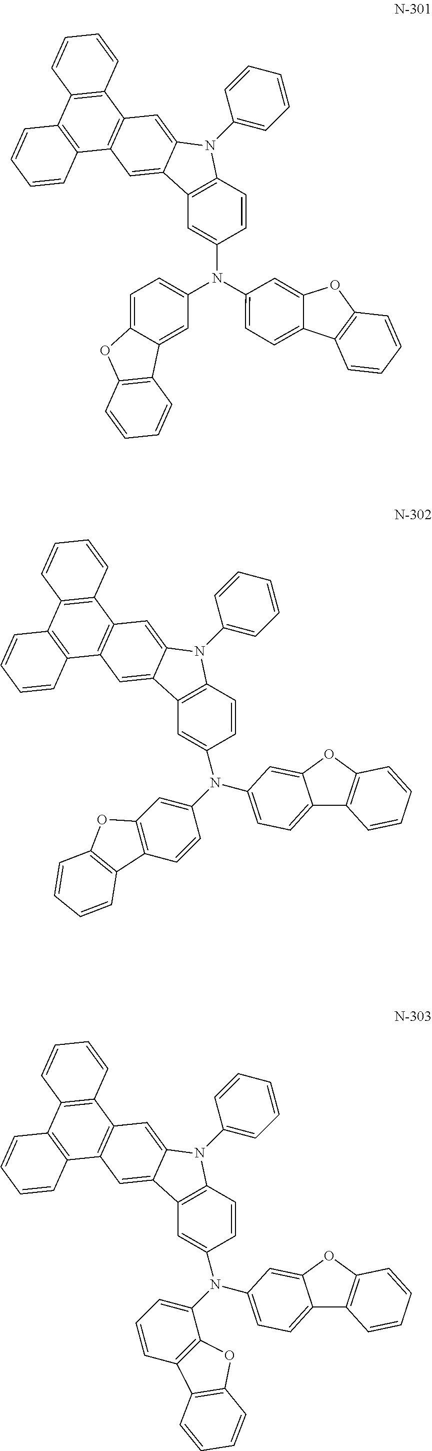 [SMILES: c1ccc(-n2c3ccc(N(c4ccc5c(c4)oc4ccccc45)c4ccc5oc6ccccc6c5c4)cc3c3cc4c5ccccc5c5ccccc5c4cc32)cc1 ; c1ccc(-n2c3ccc(N(c4ccc5c(c4)oc4ccccc45)c4ccc5c(c4)oc4ccccc45)cc3c3cc4c5ccccc5c5ccccc5c4cc32)cc1 ; c1ccc(-n2c3ccc(N(c4ccc5c(c4)oc4ccccc45)c4cccc5c4oc4ccccc45)cc3c3cc4c5ccccc5c5ccccc5c4cc32)cc1]

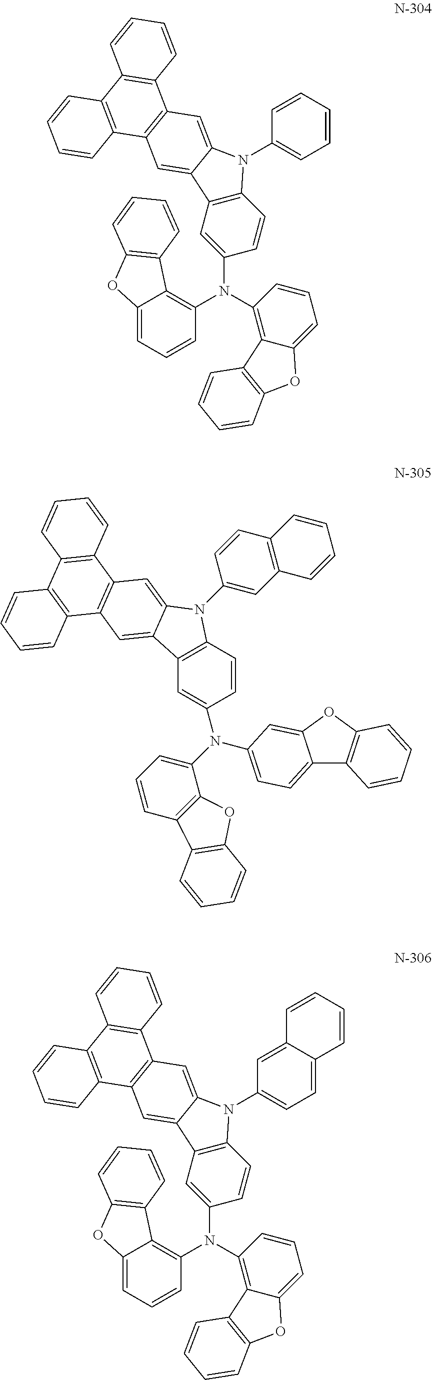 [SMILES: c1ccc(-n2c3ccc(N(c4cccc5oc6ccccc6c45)c4cccc5oc6ccccc6c45)cc3c3cc4c5ccccc5c5ccccc5c4cc32)cc1 ; c1ccc2cc(-n3c4ccc(N(c5ccc6c(c5)oc5ccccc56)c5cccc6c5oc5ccccc56)cc4c4cc5c6ccccc6c6ccccc6c5cc43)ccc2c1 ; c1ccc2cc(-n3c4ccc(N(c5cccc6oc7ccccc7c56)c5cccc6oc7ccccc7c56)cc4c4cc5c6ccccc6c6ccccc6c5cc43)ccc2c1]

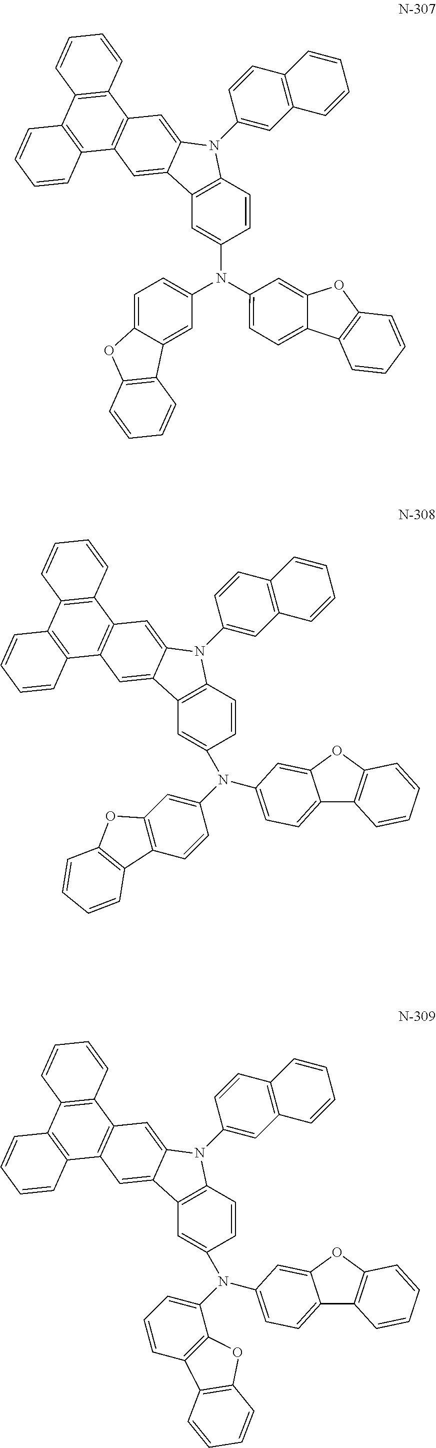 [SMILES: c1ccc2cc(-n3c4ccc(N(c5ccc6c(c5)oc5ccccc56)c5ccc6oc7ccccc7c6c5)cc4c4cc5c6ccccc6c6ccccc6c5cc43)ccc2c1 ; c1ccc2cc(-n3c4ccc(N(c5ccc6c(c5)oc5ccccc56)c5ccc6c(c5)oc5ccccc56)cc4c4cc5c6ccccc6c6ccccc6c5cc43)ccc2c1 ; c1ccc2cc(-n3c4ccc(N(c5ccc6c(c5)oc5ccccc56)c5cccc6c5oc5ccccc56)cc4c4cc5c6ccccc6c6ccccc6c5cc43)ccc2c1]

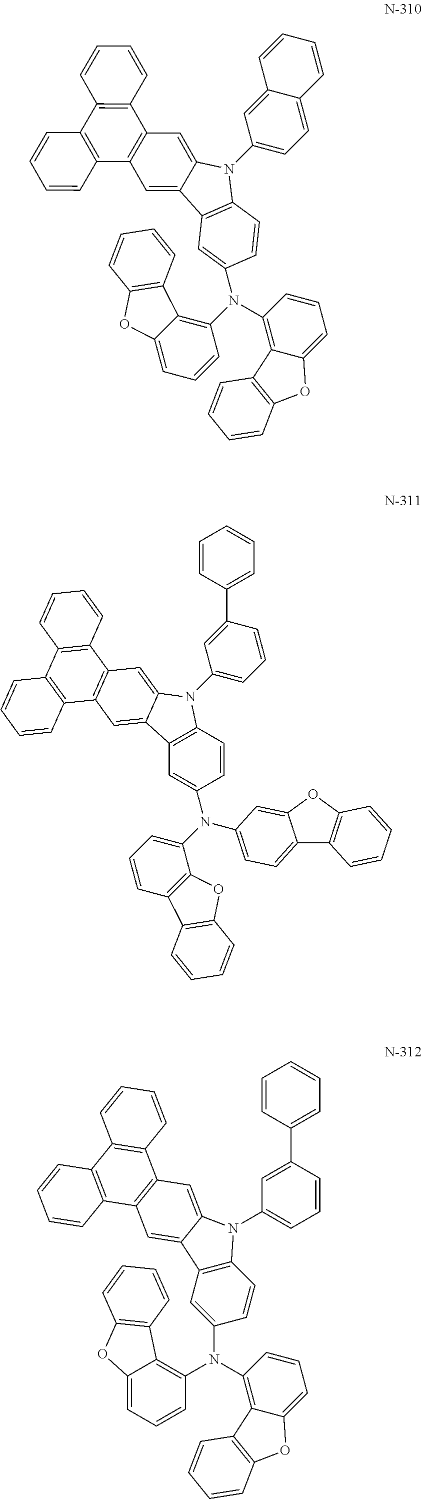 [SMILES: c1ccc2cc(-n3c4ccc(N(c5cccc6oc7ccccc7c56)c5cccc6oc7ccccc7c56)cc4c4cc5c6ccccc6c6ccccc6c5cc43)ccc2c1 ; c1ccc(-c2cccc(-n3c4ccc(N(c5ccc6c(c5)oc5ccccc56)c5cccc6c5oc5ccccc56)cc4c4cc5c6ccccc6c6ccccc6c5cc43)c2)cc1 ; c1ccc(-c2cccc(-n3c4ccc(N(c5cccc6oc7ccccc7c56)c5cccc6oc7ccccc7c56)cc4c4cc5c6ccccc6c6ccccc6c5cc43)c2)cc1]

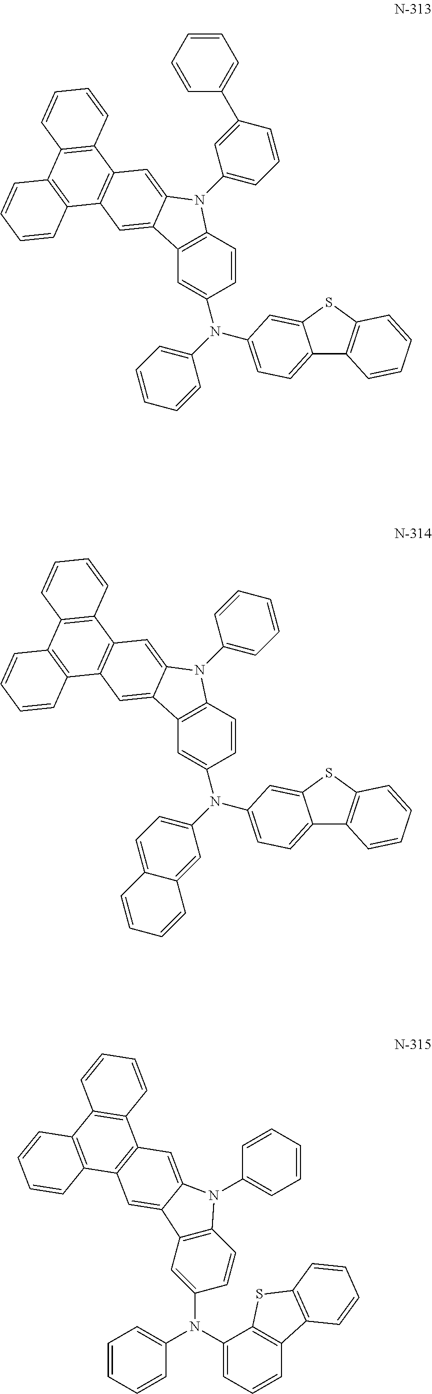 [SMILES: c1ccc(-c2cccc(-n3c4ccc(N(c5ccccc5)c5ccc6c(c5)sc5ccccc56)cc4c4cc5c6ccccc6c6ccccc6c5cc43)c2)cc1 ; c1ccc(-n2c3ccc(N(c4ccc5ccccc5c4)c4ccc5c(c4)sc4ccccc45)cc3c3cc4c5ccccc5c5ccccc5c4cc32)cc1 ; c1ccc(N(c2ccc3c(c2)c2cc4c5ccccc5c5ccccc5c4cc2n3-c2ccccc2)c2cccc3c2sc2ccccc23)cc1]

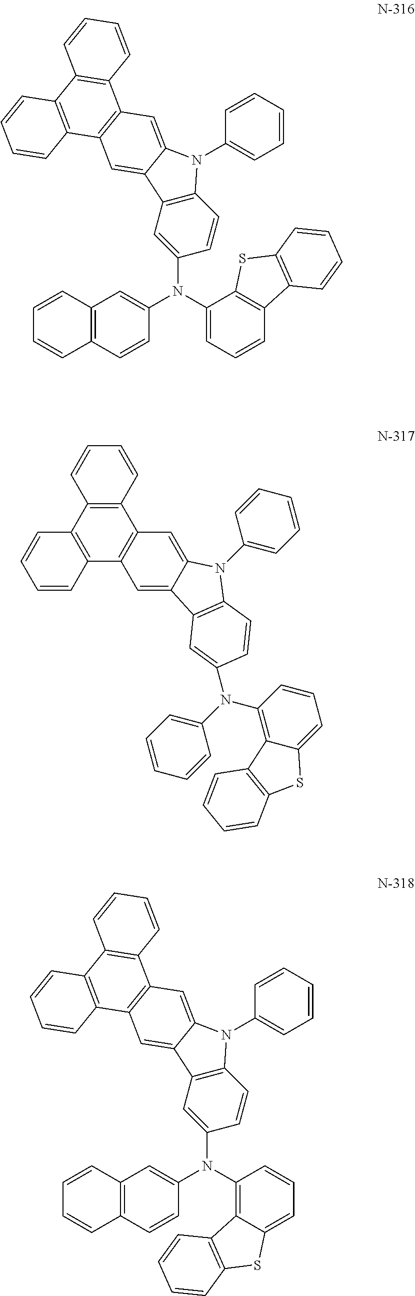 [SMILES: c1ccc(-n2c3ccc(N(c4ccc5ccccc5c4)c4cccc5c4sc4ccccc45)cc3c3cc4c5ccccc5c5ccccc5c4cc32)cc1 ; c1ccc(N(c2ccc3c(c2)c2cc4c5ccccc5c5ccccc5c4cc2n3-c2ccccc2)c2cccc3sc4ccccc4c23)cc1 ; c1ccc(-n2c3ccc(N(c4ccc5ccccc5c4)c4cccc5sc6ccccc6c45)cc3c3cc4c5ccccc5c5ccccc5c4cc32)cc1]

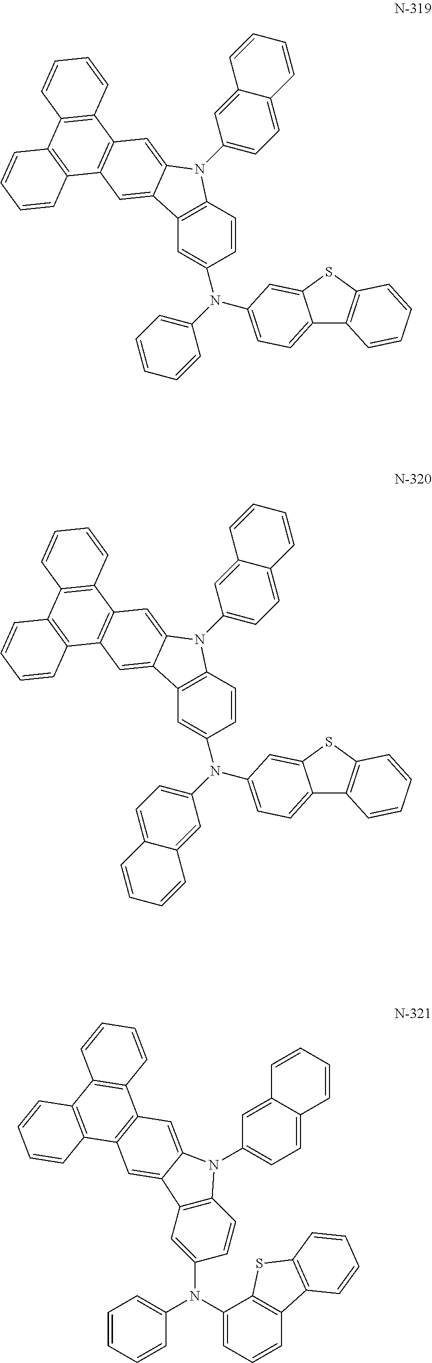 [SMILES: c1ccc(N(c2ccc3c(c2)sc2ccccc23)c2ccc3c(c2)c2cc4c5ccccc5c5ccccc5c4cc2n3-c2ccc3ccccc3c2)cc1 ; c1ccc2cc(N(c3ccc4c(c3)sc3ccccc34)c3ccc4c(c3)c3cc5c6ccccc6c6ccccc6c5cc3n4-c3ccc4ccccc4c3)ccc2c1 ; c1ccc(N(c2ccc3c(c2)c2cc4c5ccccc5c5ccccc5c4cc2n3-c2ccc3ccccc3c2)c2cccc3c2sc2ccccc23)cc1]

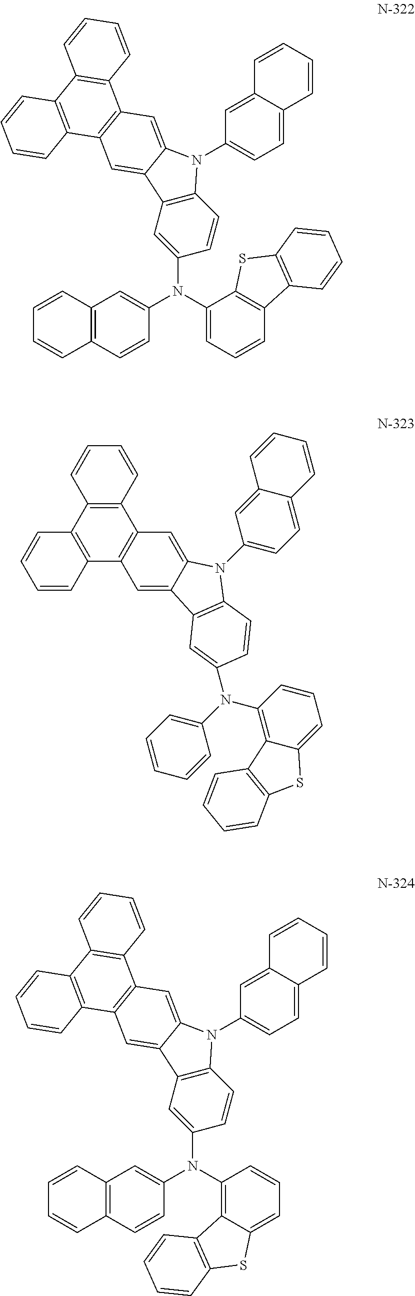 [SMILES: c1ccc2cc(N(c3ccc4c(c3)c3cc5c6ccccc6c6ccccc6c5cc3n4-c3ccc4ccccc4c3)c3cccc4c3sc3ccccc34)ccc2c1 ; c1ccc(N(c2ccc3c(c2)c2cc4c5ccccc5c5ccccc5c4cc2n3-c2ccc3ccccc3c2)c2cccc3sc4ccccc4c23)cc1 ; c1ccc2cc(N(c3ccc4c(c3)c3cc5c6ccccc6c6ccccc6c5cc3n4-c3ccc4ccccc4c3)c3cccc4sc5ccccc5c34)ccc2c1]

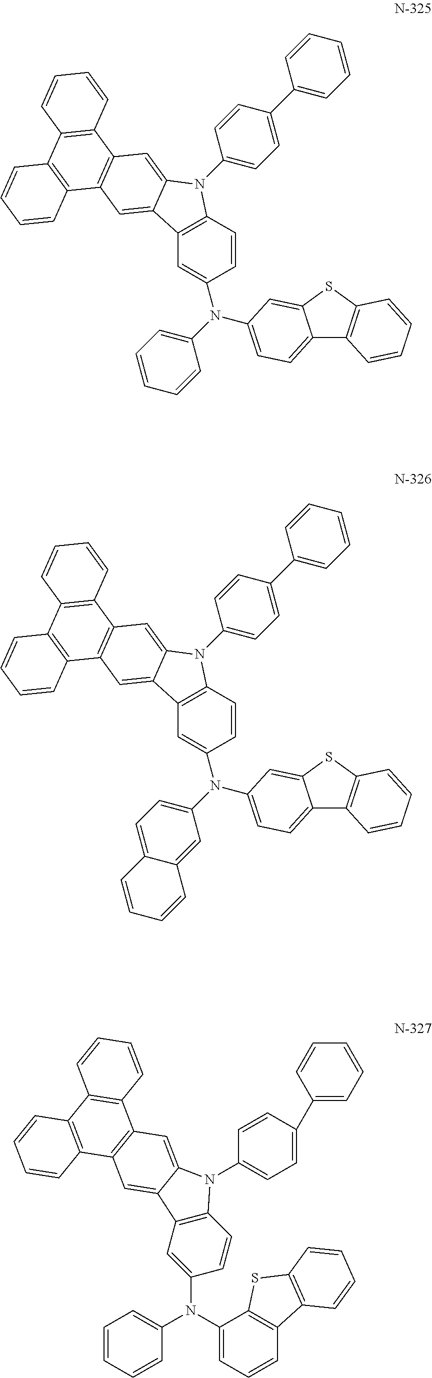 [SMILES: c1ccc(-c2ccc(-n3c4ccc(N(c5ccccc5)c5ccc6c(c5)sc5ccccc56)cc4c4cc5c6ccccc6c6ccccc6c5cc43)cc2)cc1 ; c1ccc(-c2ccc(-n3c4ccc(N(c5ccc6ccccc6c5)c5ccc6c(c5)sc5ccccc56)cc4c4cc5c6ccccc6c6ccccc6c5cc43)cc2)cc1 ; c1ccc(-c2ccc(-n3c4ccc(N(c5ccccc5)c5cccc6c5sc5ccccc56)cc4c4cc5c6ccccc6c6ccccc6c5cc43)cc2)cc1]

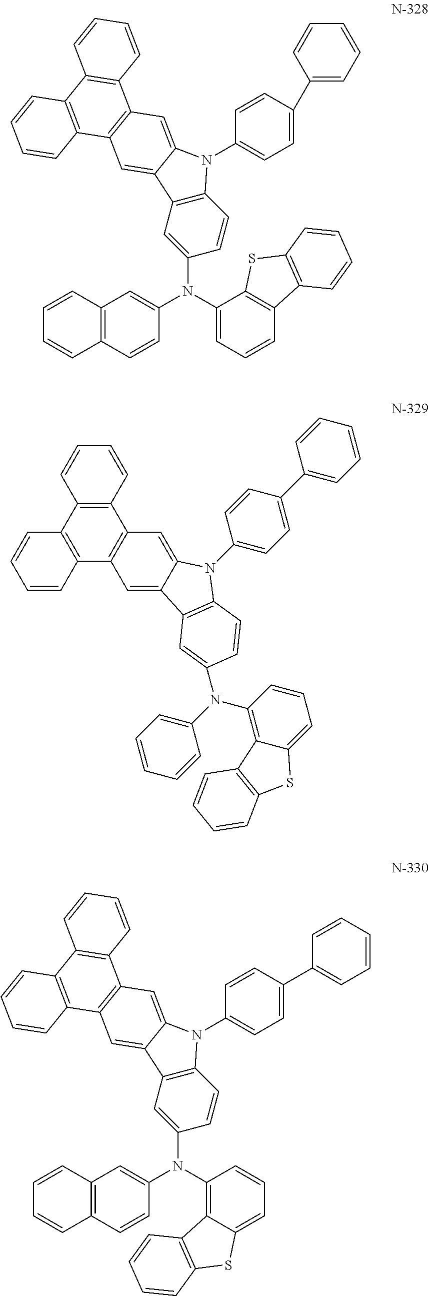 [SMILES: c1ccc(-c2ccc(-n3c4ccc(N(c5ccc6ccccc6c5)c5cccc6c5sc5ccccc56)cc4c4cc5c6ccccc6c6ccccc6c5cc43)cc2)cc1 ; c1ccc(-c2ccc(-n3c4ccc(N(c5ccccc5)c5cccc6sc7ccccc7c56)cc4c4cc5c6ccccc6c6ccccc6c5cc43)cc2)cc1 ; c1ccc(-c2ccc(-n3c4ccc(N(c5ccc6ccccc6c5)c5cccc6sc7ccccc7c56)cc4c4cc5c6ccccc6c6ccccc6c5cc43)cc2)cc1]

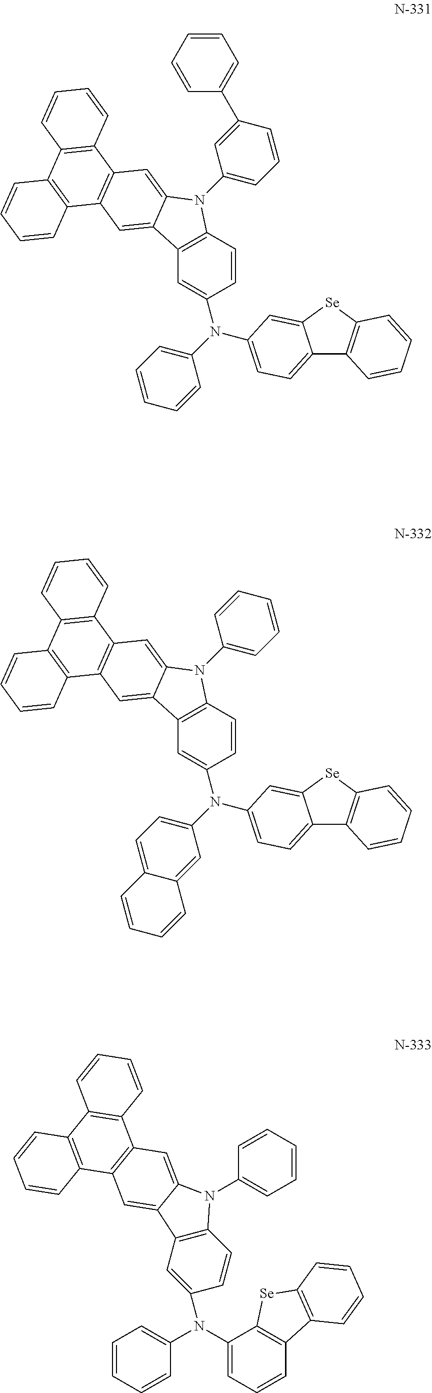 [SMILES: c1ccc(-c2cccc(-n3c4ccc(N(c5ccccc5)c5ccc6c(c5)[se]c5ccccc56)cc4c4cc5c6ccccc6c6ccccc6c5cc43)c2)cc1 ; c1ccc(-n2c3ccc(N(c4ccc5ccccc5c4)c4ccc5c(c4)[se]c4ccccc45)cc3c3cc4c5ccccc5c5ccccc5c4cc32)cc1 ; c1ccc(N(c2ccc3c(c2)c2cc4c5ccccc5c5ccccc5c4cc2n3-c2ccccc2)c2cccc3c2[se]c2ccccc23)cc1]

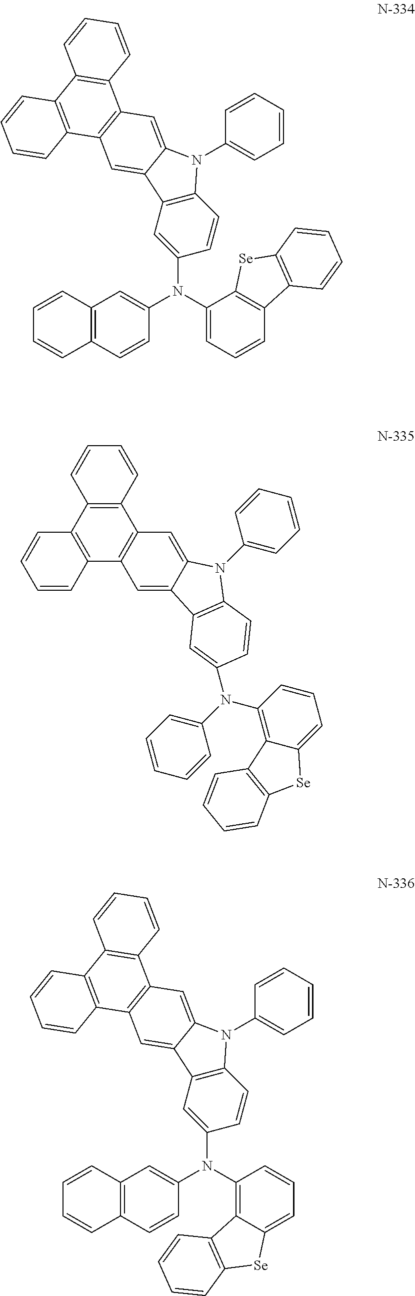 [SMILES: c1ccc(-n2c3ccc(N(c4ccc5ccccc5c4)c4cccc5c4[se]c4ccccc45)cc3c3cc4c5ccccc5c5ccccc5c4cc32)cc1 ; c1ccc(N(c2ccc3c(c2)c2cc4c5ccccc5c5ccccc5c4cc2n3-c2ccccc2)c2cccc3[se]c4ccccc4c23)cc1 ; c1ccc(-n2c3ccc(N(c4ccc5ccccc5c4)c4cccc5[se]c6ccccc6c45)cc3c3cc4c5ccccc5c5ccccc5c4cc32)cc1]

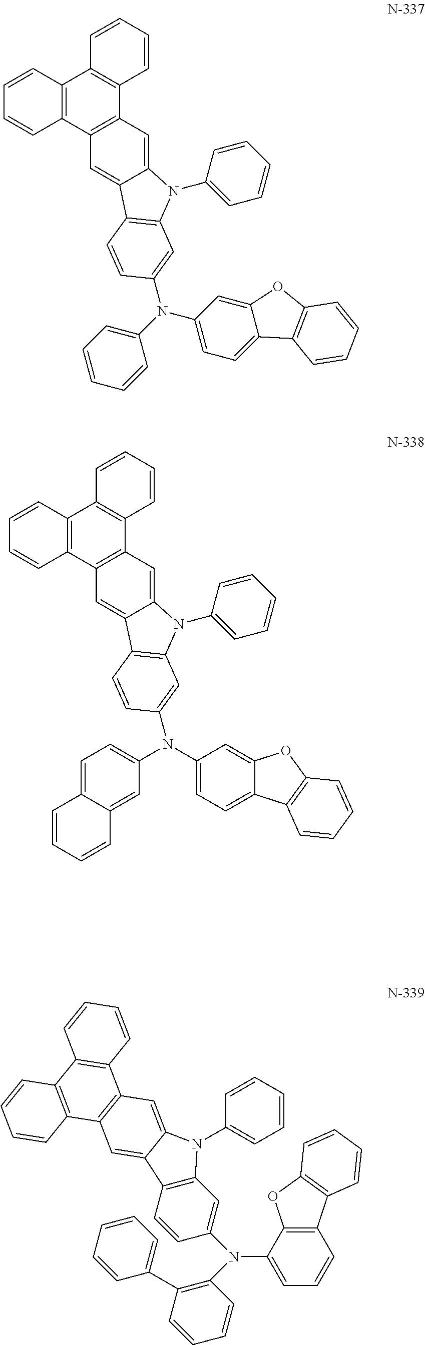 [SMILES: c1ccc(N(c2ccc3c(c2)oc2ccccc23)c2ccc3c4cc5c6ccccc6c6ccccc6c5cc4n(-c4ccccc4)c3c2)cc1 ; c1ccc(-n2c3cc(N(c4ccc5ccccc5c4)c4ccc5c(c4)oc4ccccc45)ccc3c3cc4c5ccccc5c5ccccc5c4cc32)cc1 ; c1ccc(-c2ccccc2N(c2ccc3c4cc5c6ccccc6c6ccccc6c5cc4n(-c4ccccc4)c3c2)c2cccc3c2oc2ccccc23)cc1]

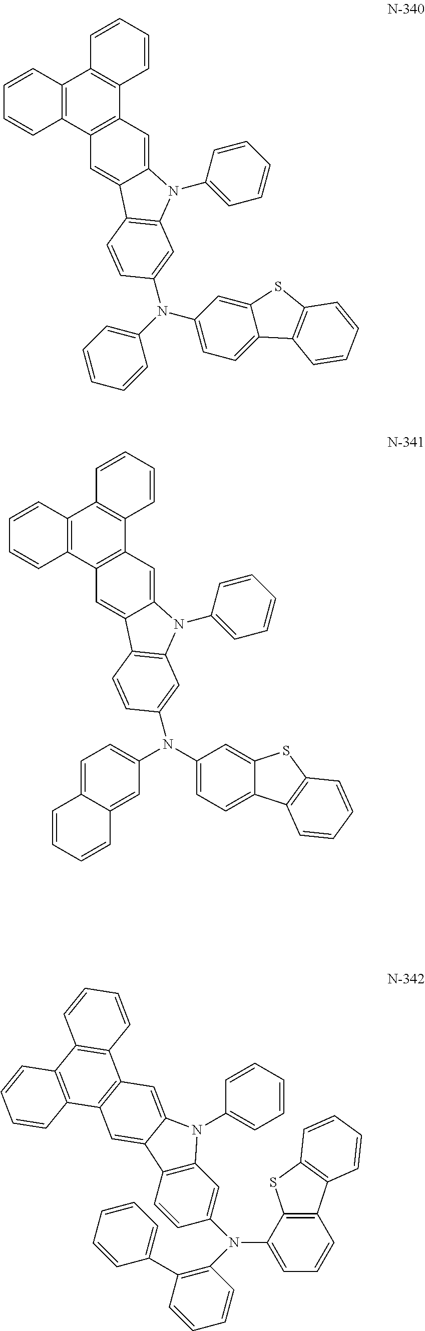 [SMILES: c1ccc(N(c2ccc3c(c2)sc2ccccc23)c2ccc3c4cc5c6ccccc6c6ccccc6c5cc4n(-c4ccccc4)c3c2)cc1 ; c1ccc(-n2c3cc(N(c4ccc5ccccc5c4)c4ccc5c(c4)sc4ccccc45)ccc3c3cc4c5ccccc5c5ccccc5c4cc32)cc1 ; c1ccc(-c2ccccc2N(c2ccc3c4cc5c6ccccc6c6ccccc6c5cc4n(-c4ccccc4)c3c2)c2cccc3c2sc2ccccc23)cc1]

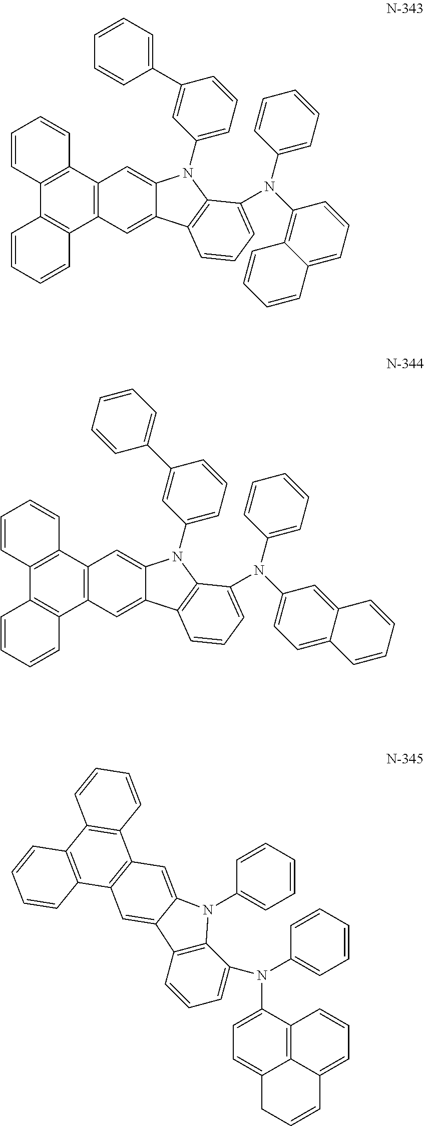 [SMILES: c1ccc(-c2cccc(-n3c4cc5c6ccccc6c6ccccc6c5cc4c4cccc(N(c5ccccc5)c5cccc6ccccc56)c43)c2)cc1 ; c1ccc(-c2cccc(-n3c4cc5c6ccccc6c6ccccc6c5cc4c4cccc(N(c5ccccc5)c5ccc6ccccc6c5)c43)c2)cc1 ; C1=Cc2cccc3c(N(c4ccccc4)c4cccc5c6cc7c8ccccc8c8ccccc8c7cc6n(-c6ccccc6)c45)ccc(c23)C1]

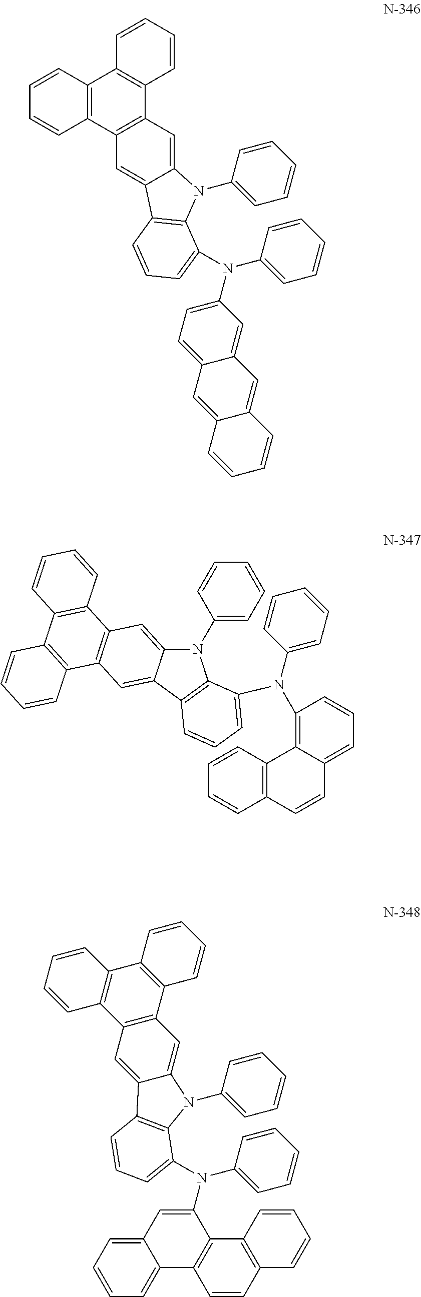 [SMILES: c1ccc(N(c2ccc3cc4ccccc4cc3c2)c2cccc3c4cc5c6ccccc6c6ccccc6c5cc4n(-c4ccccc4)c23)cc1 ; c1ccc(N(c2cccc3ccc4ccccc4c23)c2cccc3c4cc5c6ccccc6c6ccccc6c5cc4n(-c4ccccc4)c23)cc1 ; c1ccc(N(c2cc3ccccc3c3ccc4ccccc4c23)c2cccc3c4cc5c6ccccc6c6ccccc6c5cc4n(-c4ccccc4)c23)cc1]

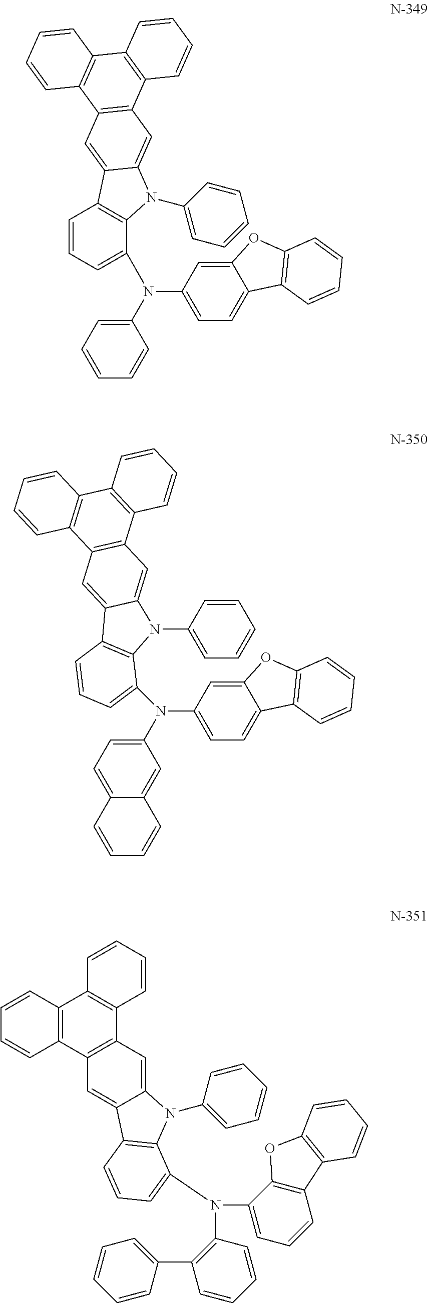 [SMILES: c1ccc(N(c2ccc3c(c2)oc2ccccc23)c2cccc3c4cc5c6ccccc6c6ccccc6c5cc4n(-c4ccccc4)c23)cc1 ; c1ccc(-n2c3cc4c5ccccc5c5ccccc5c4cc3c3cccc(N(c4ccc5ccccc5c4)c4ccc5c(c4)oc4ccccc45)c32)cc1 ; c1ccc(-c2ccccc2N(c2cccc3c2oc2ccccc23)c2cccc3c4cc5c6ccccc6c6ccccc6c5cc4n(-c4ccccc4)c23)cc1]

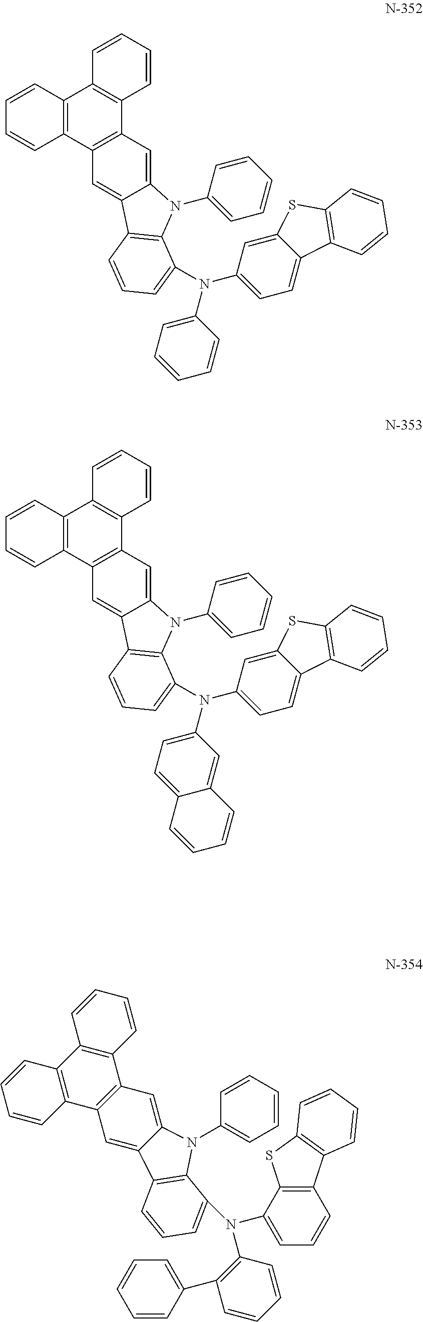 [SMILES: c1ccc(N(c2ccc3c(c2)sc2ccccc23)c2cccc3c4cc5c6ccccc6c6ccccc6c5cc4n(-c4ccccc4)c23)cc1 ; c1ccc(-n2c3cc4c5ccccc5c5ccccc5c4cc3c3cccc(N(c4ccc5ccccc5c4)c4ccc5c(c4)sc4ccccc45)c32)cc1 ; c1ccc(-c2ccccc2N(c2cccc3c2sc2ccccc23)c2cccc3c4cc5c6ccccc6c6ccccc6c5cc4n(-c4ccccc4)c23)cc1]

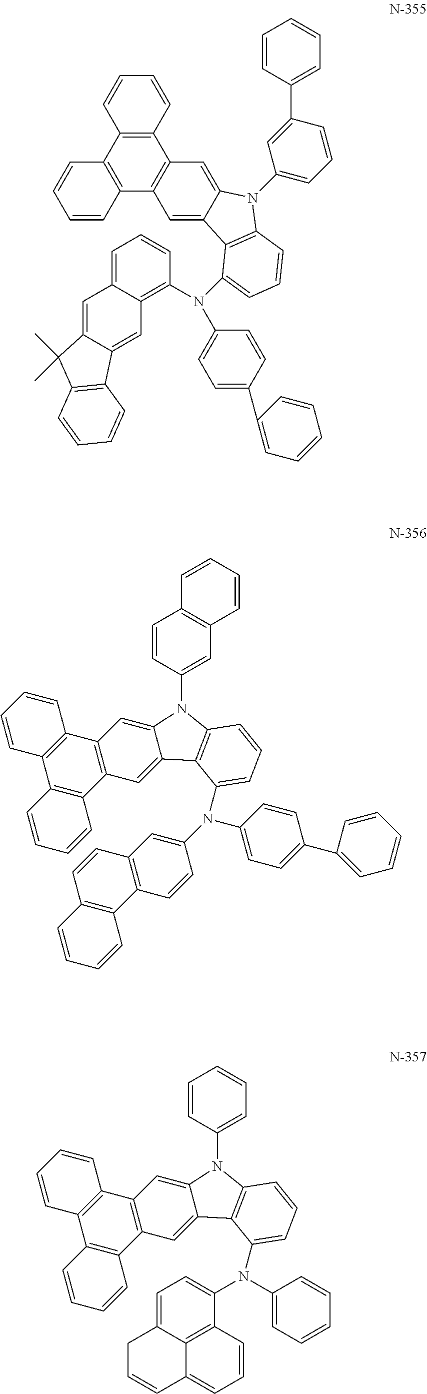 [SMILES: CC1(C)c2ccccc2-c2cc3c(N(c4ccc(-c5ccccc5)cc4)c4cccc5c4c4cc6c7ccccc7c7ccccc7c6cc4n5-c4cccc(-c5ccccc5)c4)cccc3cc21 ; c1ccc(-c2ccc(N(c3ccc4c(ccc5ccccc54)c3)c3cccc4c3c3cc5c6ccccc6c6ccccc6c5cc3n4-c3ccc4ccccc4c3)cc2)cc1 ; C1=Cc2cccc3c(N(c4ccccc4)c4cccc5c4c4cc6c7ccccc7c7ccccc7c6cc4n5-c4ccccc4)ccc(c23)C1]

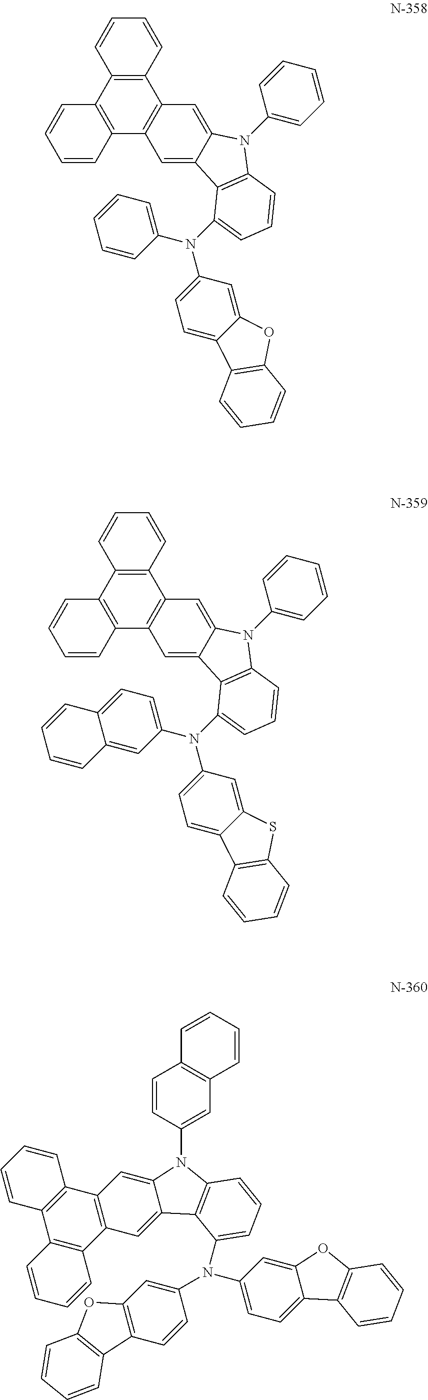 [SMILES: c1ccc(N(c2ccc3c(c2)oc2ccccc23)c2cccc3c2c2cc4c5ccccc5c5ccccc5c4cc2n3-c2ccccc2)cc1 ; c1ccc(-n2c3cc4c5ccccc5c5ccccc5c4cc3c3c(N(c4ccc5ccccc5c4)c4ccc5c(c4)sc4ccccc45)cccc32)cc1 ; c1ccc2cc(-n3c4cc5c6ccccc6c6ccccc6c5cc4c4c(N(c5ccc6c(c5)oc5ccccc56)c5ccc6c(c5)oc5ccccc56)cccc43)ccc2c1]

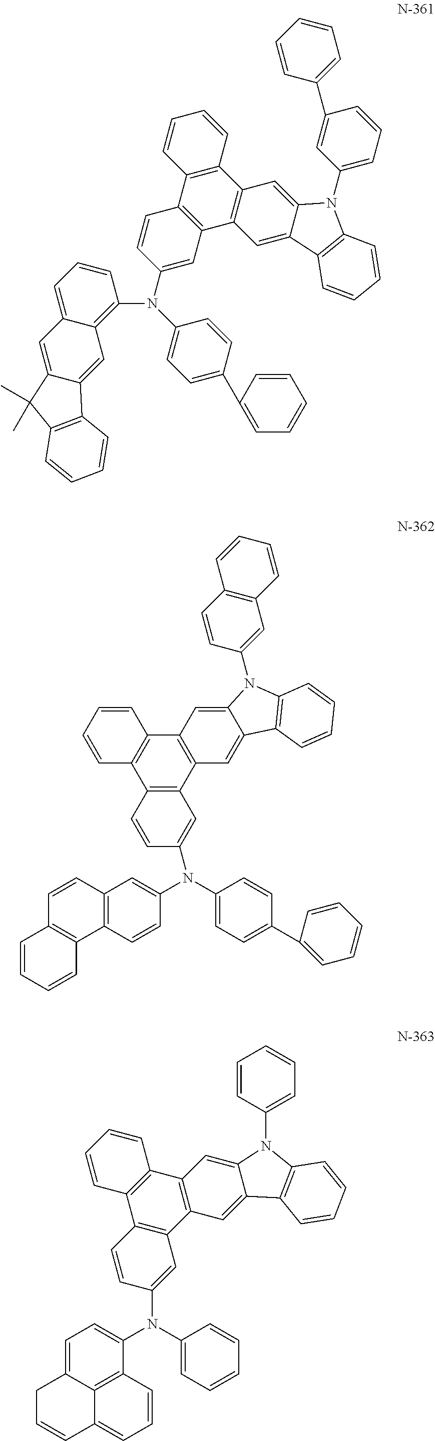 [SMILES: CC1(C)c2ccccc2-c2cc3c(N(c4ccc(-c5ccccc5)cc4)c4ccc5c6ccccc6c6cc7c(cc6c5c4)c4ccccc4n7-c4cccc(-c5ccccc5)c4)cccc3cc21 ; c1ccc(-c2ccc(N(c3ccc4c(ccc5ccccc54)c3)c3ccc4c5ccccc5c5cc6c(cc5c4c3)c3ccccc3n6-c3ccc4ccccc4c3)cc2)cc1 ; C1=Cc2cccc3c(N(c4ccccc4)c4ccc5c6ccccc6c6cc7c(cc6c5c4)c4ccccc4n7-c4ccccc4)ccc(c23)C1]

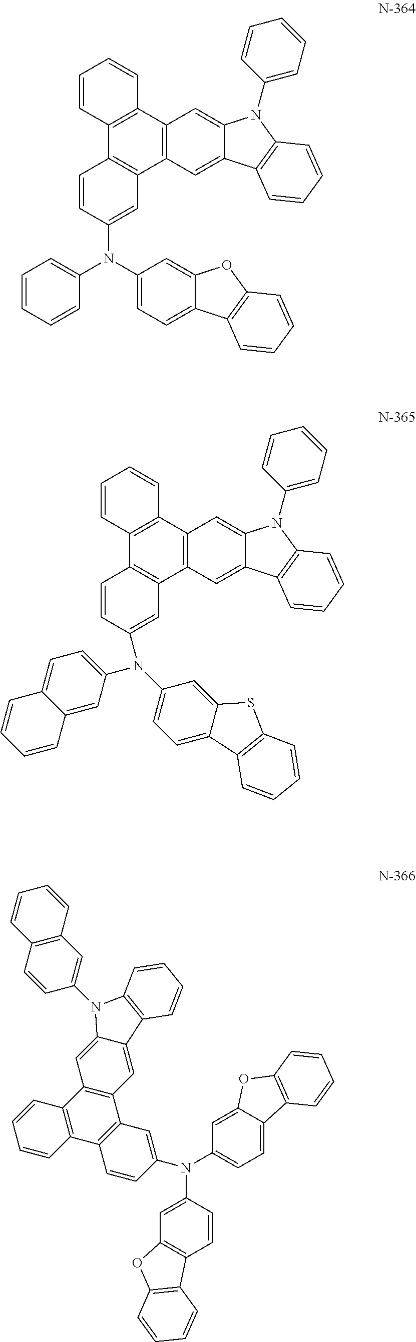 [SMILES: c1ccc(N(c2ccc3c(c2)oc2ccccc23)c2ccc3c4ccccc4c4cc5c(cc4c3c2)c2ccccc2n5-c2ccccc2)cc1 ; c1ccc(-n2c3ccccc3c3cc4c5cc(N(c6ccc7ccccc7c6)c6ccc7c(c6)sc6ccccc67)ccc5c5ccccc5c4cc32)cc1 ; c1ccc2cc(-n3c4ccccc4c4cc5c6cc(N(c7ccc8c(c7)oc7ccccc78)c7ccc8c(c7)oc7ccccc78)ccc6c6ccccc6c5cc43)ccc2c1]

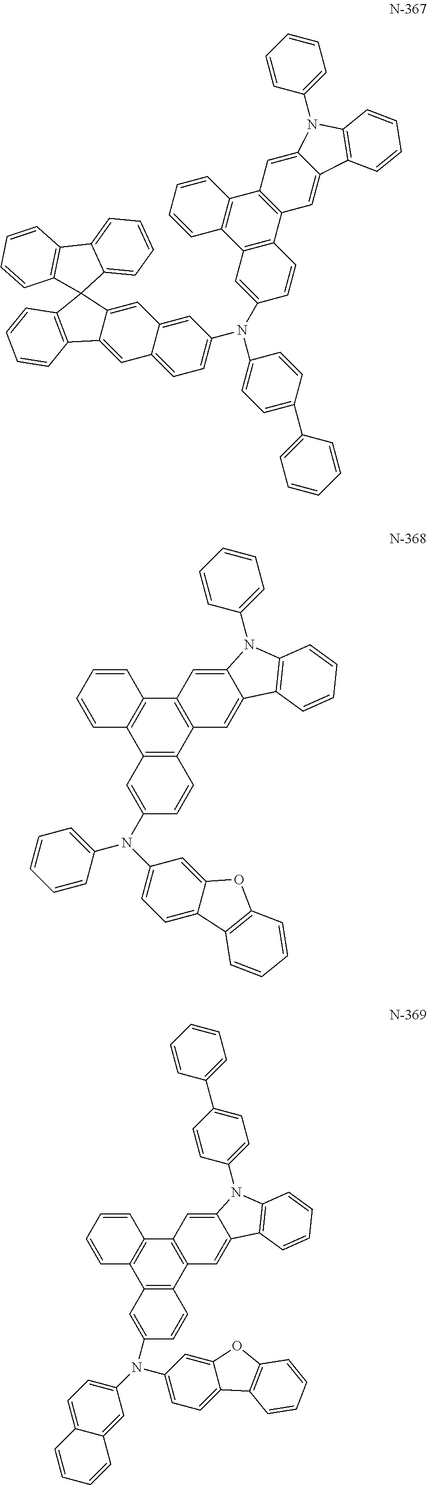 [SMILES: c1ccc(-c2ccc(N(c3ccc4cc5c(cc4c3)C3(c4ccccc4-c4ccccc43)c3ccccc3-5)c3ccc4c(c3)c3ccccc3c3cc5c(cc43)c3ccccc3n5-c3ccccc3)cc2)cc1 ; c1ccc(N(c2ccc3c(c2)oc2ccccc23)c2ccc3c(c2)c2ccccc2c2cc4c(cc32)c2ccccc2n4-c2ccccc2)cc1 ; c1ccc(-c2ccc(-n3c4ccccc4c4cc5c6ccc(N(c7ccc8ccccc8c7)c7ccc8c(c7)oc7ccccc78)cc6c6ccccc6c5cc43)cc2)cc1]

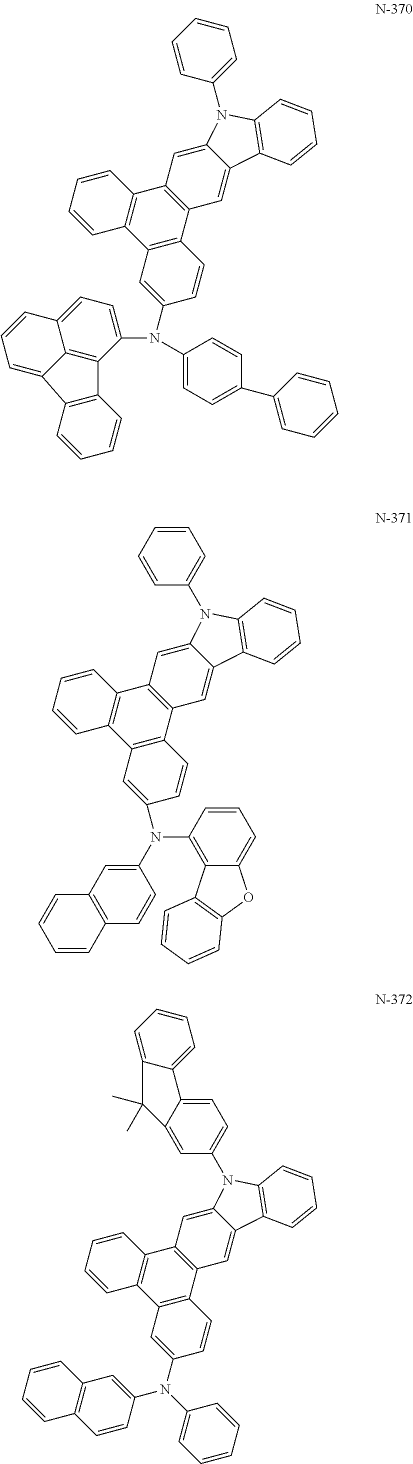 [SMILES: c1ccc(-c2ccc(N(c3ccc4c(c3)c3ccccc3c3cc5c(cc43)c3ccccc3n5-c3ccccc3)c3ccc4cccc5c4c3-c3ccccc3-5)cc2)cc1 ; c1ccc(-n2c3ccccc3c3cc4c5ccc(N(c6ccc7ccccc7c6)c6cccc7oc8ccccc8c67)cc5c5ccccc5c4cc32)cc1 ; CC1(C)c2ccccc2-c2ccc(-n3c4ccccc4c4cc5c6ccc(N(c7ccccc7)c7ccc8ccccc8c7)cc6c6ccccc6c5cc43)cc21]

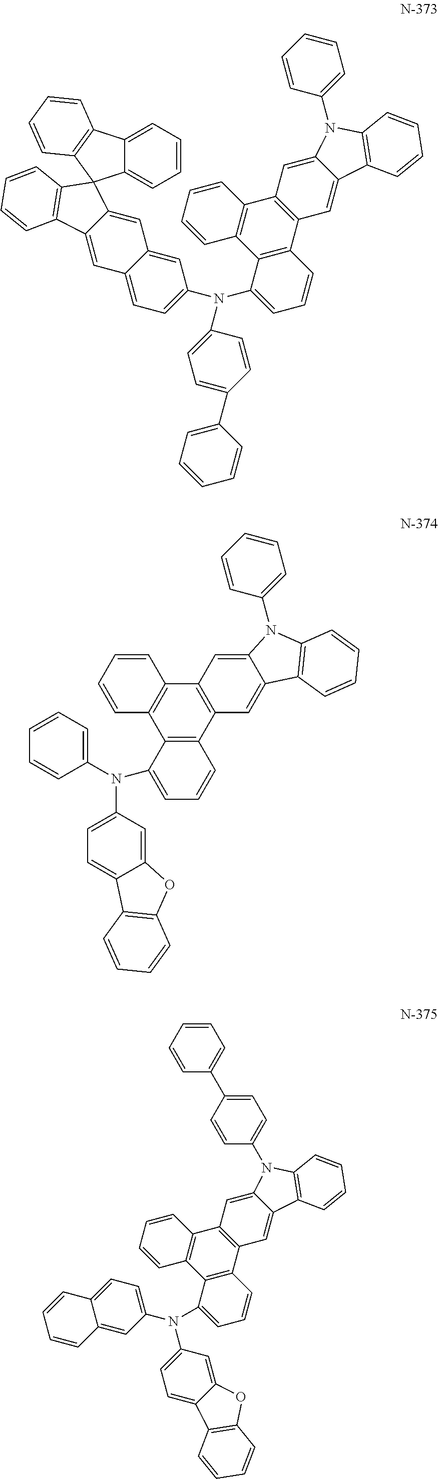 [SMILES: c1ccc(-c2ccc(N(c3ccc4cc5c(cc4c3)C3(c4ccccc4-c4ccccc43)c3ccccc3-5)c3cccc4c5cc6c7ccccc7n(-c7ccccc7)c6cc5c5ccccc5c34)cc2)cc1 ; c1ccc(N(c2ccc3c(c2)oc2ccccc23)c2cccc3c4cc5c6ccccc6n(-c6ccccc6)c5cc4c4ccccc4c23)cc1 ; c1ccc(-c2ccc(-n3c4ccccc4c4cc5c(cc43)c3ccccc3c3c(N(c4ccc6ccccc6c4)c4ccc6c(c4)oc4ccccc46)cccc53)cc2)cc1]

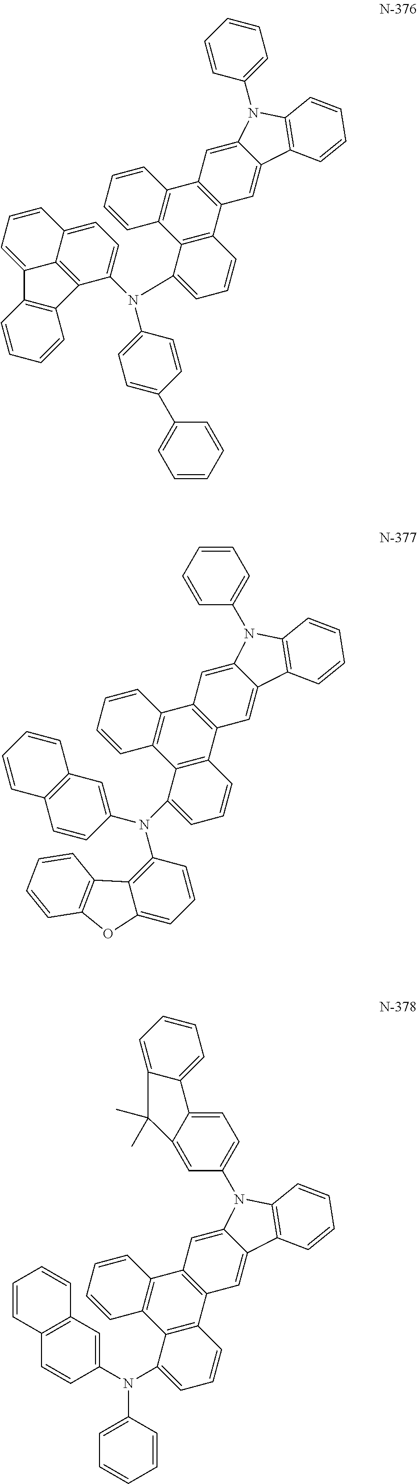 [SMILES: c1ccc(-c2ccc(N(c3ccc4cccc5c4c3-c3ccccc3-5)c3cccc4c5cc6c7ccccc7n(-c7ccccc7)c6cc5c5ccccc5c34)cc2)cc1 ; c1ccc(-n2c3ccccc3c3cc4c(cc32)c2ccccc2c2c(N(c3ccc5ccccc5c3)c3cccc5oc6ccccc6c35)cccc42)cc1 ; CC1(C)c2ccccc2-c2ccc(-n3c4ccccc4c4cc5c(cc43)c3ccccc3c3c(N(c4ccccc4)c4ccc6ccccc6c4)cccc53)cc21]

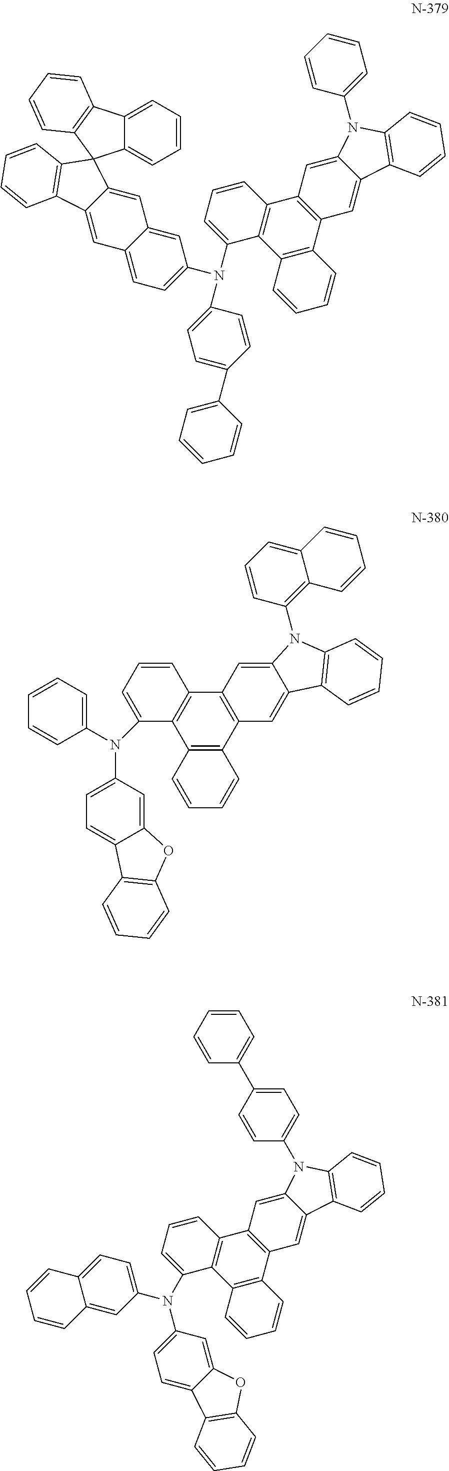 [SMILES: c1ccc(-c2ccc(N(c3ccc4cc5c(cc4c3)C3(c4ccccc4-c4ccccc43)c3ccccc3-5)c3cccc4c5cc6c(cc5c5ccccc5c34)c3ccccc3n6-c3ccccc3)cc2)cc1 ; c1ccc(N(c2ccc3c(c2)oc2ccccc23)c2cccc3c4cc5c(cc4c4ccccc4c23)c2ccccc2n5-c2cccc3ccccc23)cc1 ; c1ccc(-c2ccc(-n3c4ccccc4c4cc5c6ccccc6c6c(N(c7ccc8ccccc8c7)c7ccc8c(c7)oc7ccccc78)cccc6c5cc43)cc2)cc1]

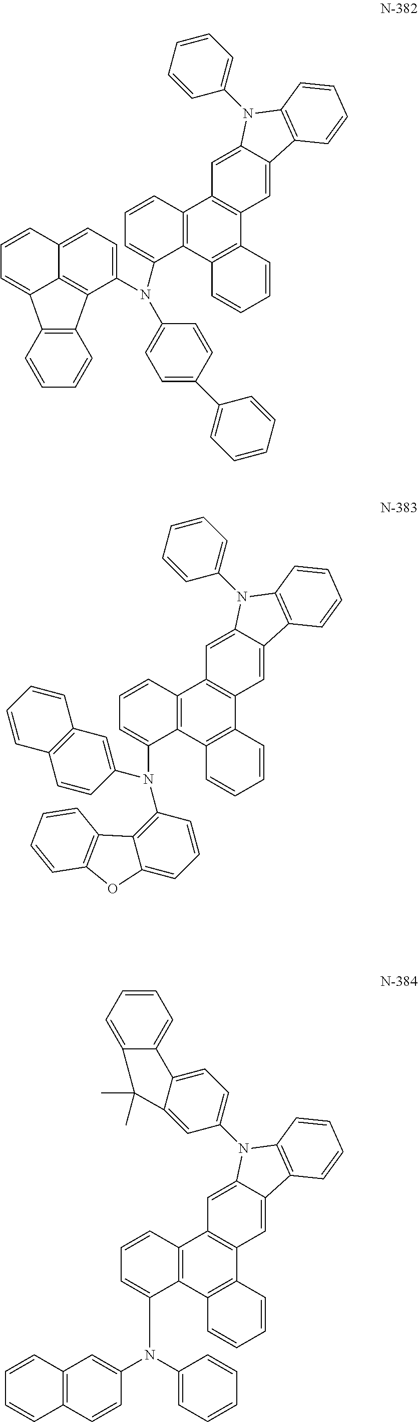 [SMILES: c1ccc(-c2ccc(N(c3ccc4cccc5c4c3-c3ccccc3-5)c3cccc4c5cc6c(cc5c5ccccc5c34)c3ccccc3n6-c3ccccc3)cc2)cc1 ; c1ccc(-n2c3ccccc3c3cc4c5ccccc5c5c(N(c6ccc7ccccc7c6)c6cccc7oc8ccccc8c67)cccc5c4cc32)cc1 ; CC1(C)c2ccccc2-c2ccc(-n3c4ccccc4c4cc5c6ccccc6c6c(N(c7ccccc7)c7ccc8ccccc8c7)cccc6c5cc43)cc21]

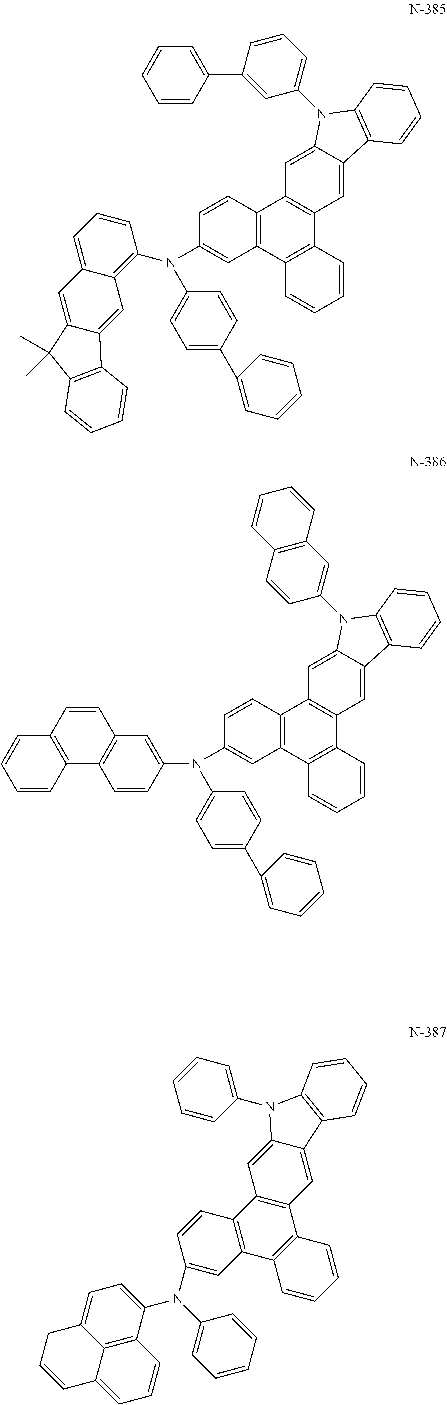 [SMILES: CC1(C)c2ccccc2-c2cc3c(N(c4ccc(-c5ccccc5)cc4)c4ccc5c(c4)c4ccccc4c4cc6c7ccccc7n(-c7cccc(-c8ccccc8)c7)c6cc54)cccc3cc21 ; c1ccc(-c2ccc(N(c3ccc4c(ccc5ccccc54)c3)c3ccc4c(c3)c3ccccc3c3cc5c6ccccc6n(-c6ccc7ccccc7c6)c5cc43)cc2)cc1 ; C1=Cc2cccc3c(N(c4ccccc4)c4ccc5c(c4)c4ccccc4c4cc6c7ccccc7n(-c7ccccc7)c6cc54)ccc(c23)C1]

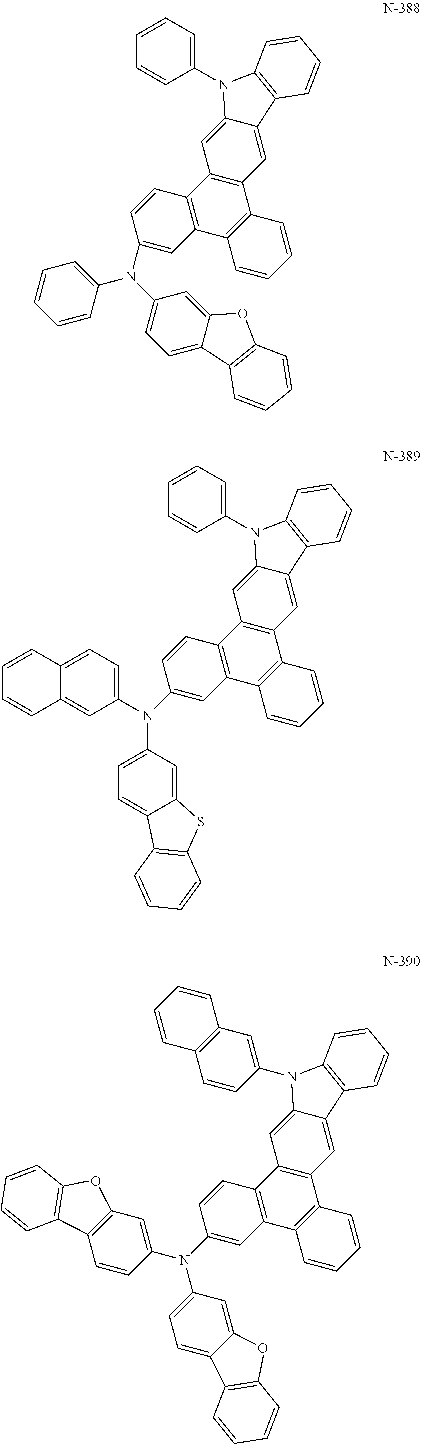 [SMILES: c1ccc(N(c2ccc3c(c2)oc2ccccc23)c2ccc3c(c2)c2ccccc2c2cc4c5ccccc5n(-c5ccccc5)c4cc32)cc1 ; c1ccc(-n2c3ccccc3c3cc4c5ccccc5c5cc(N(c6ccc7ccccc7c6)c6ccc7c(c6)sc6ccccc67)ccc5c4cc32)cc1 ; c1ccc2cc(-n3c4ccccc4c4cc5c6ccccc6c6cc(N(c7ccc8c(c7)oc7ccccc78)c7ccc8c(c7)oc7ccccc78)ccc6c5cc43)ccc2c1]

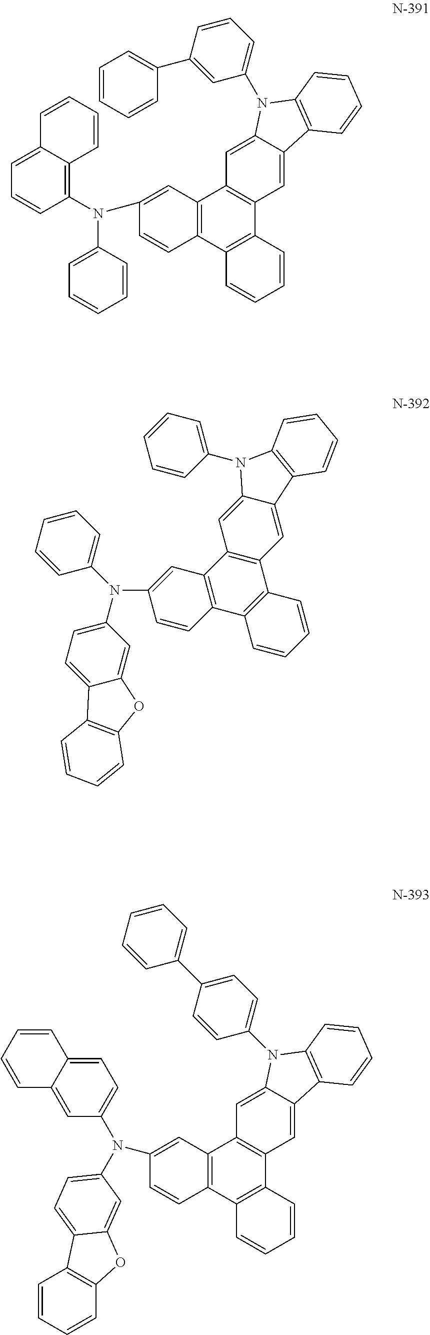 [SMILES: c1ccc(-c2cccc(-n3c4ccccc4c4cc5c6ccccc6c6ccc(N(c7ccccc7)c7cccc8ccccc78)cc6c5cc43)c2)cc1 ; c1ccc(N(c2ccc3c(c2)oc2ccccc23)c2ccc3c4ccccc4c4cc5c6ccccc6n(-c6ccccc6)c5cc4c3c2)cc1 ; c1ccc(-c2ccc(-n3c4ccccc4c4cc5c6ccccc6c6ccc(N(c7ccc8ccccc8c7)c7ccc8c(c7)oc7ccccc78)cc6c5cc43)cc2)cc1]

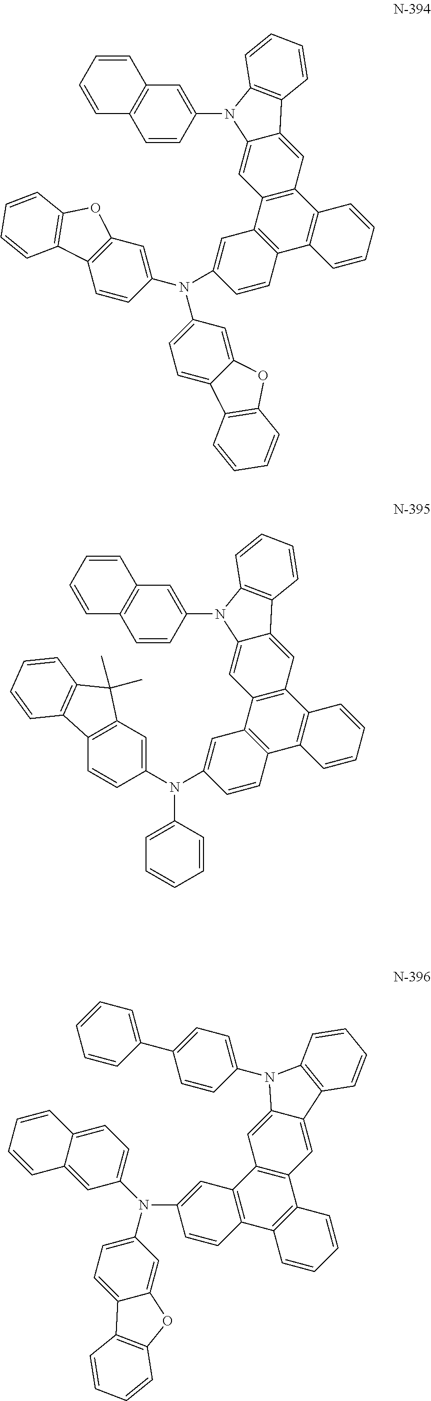 [SMILES: c1ccc2cc(-n3c4ccccc4c4cc5c6ccccc6c6ccc(N(c7ccc8c(c7)oc7ccccc78)c7ccc8c(c7)oc7ccccc78)cc6c5cc43)ccc2c1 ; CC1(C)c2ccccc2-c2ccc(N(c3ccccc3)c3ccc4c5ccccc5c5cc6c7ccccc7n(-c7ccc8ccccc8c7)c6cc5c4c3)cc21 ; c1ccc(-c2ccc(-n3c4ccccc4c4cc5c6ccccc6c6ccc(N(c7ccc8ccccc8c7)c7ccc8c(c7)oc7ccccc78)cc6c5cc43)cc2)cc1]

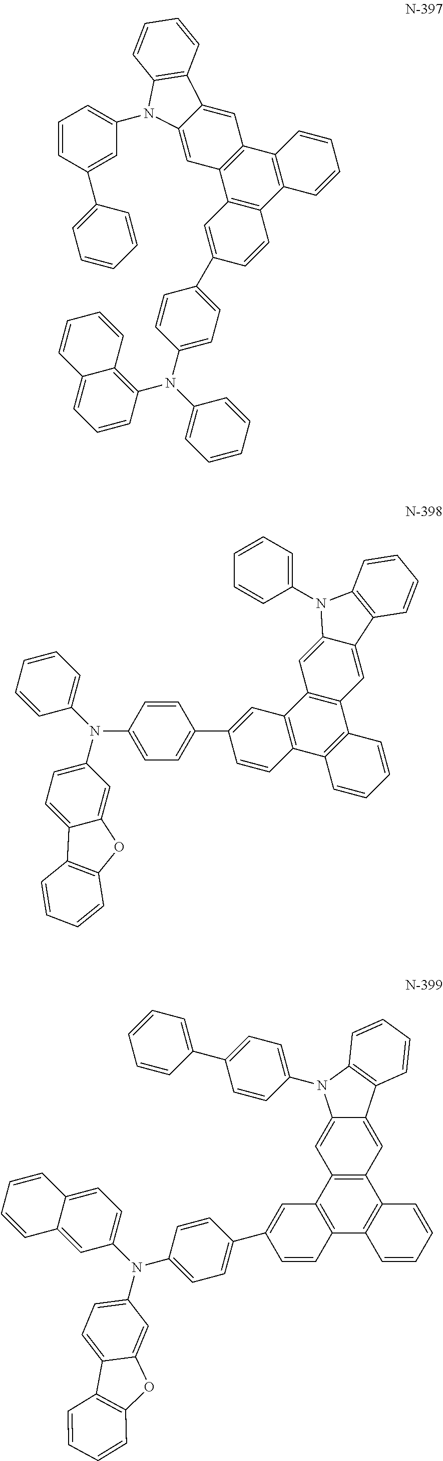 [SMILES: c1ccc(-c2cccc(-n3c4ccccc4c4cc5c6ccccc6c6ccc(-c7ccc(N(c8ccccc8)c8cccc9ccccc89)cc7)cc6c5cc43)c2)cc1 ; c1ccc(N(c2ccc(-c3ccc4c5ccccc5c5cc6c7ccccc7n(-c7ccccc7)c6cc5c4c3)cc2)c2ccc3c(c2)oc2ccccc23)cc1 ; c1ccc(-c2ccc(-n3c4ccccc4c4cc5c6ccccc6c6ccc(-c7ccc(N(c8ccc9ccccc9c8)c8ccc9c(c8)oc8ccccc89)cc7)cc6c5cc43)cc2)cc1]

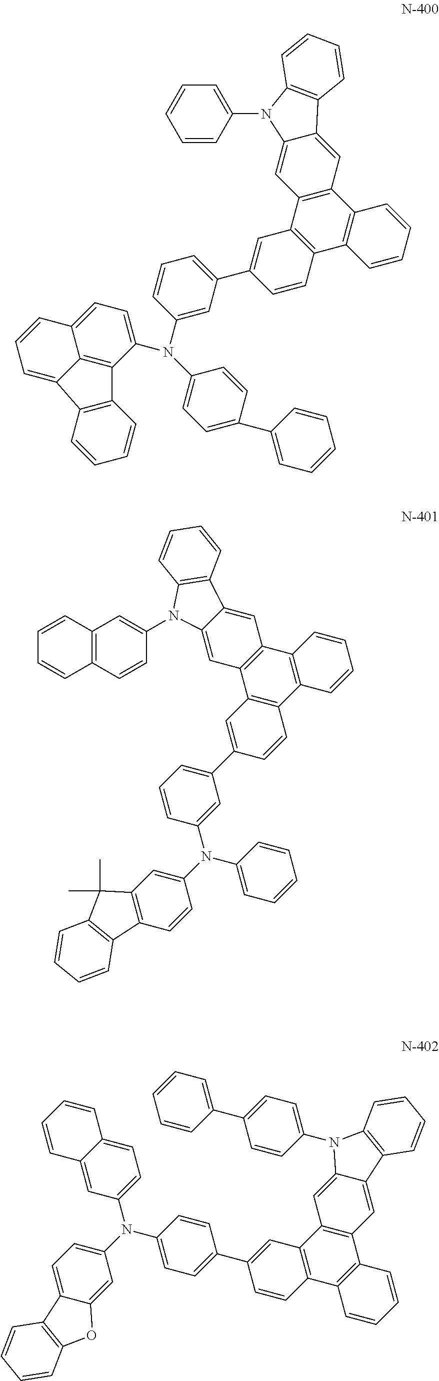 [SMILES: c1ccc(-c2ccc(N(c3cccc(-c4ccc5c6ccccc6c6cc7c8ccccc8n(-c8ccccc8)c7cc6c5c4)c3)c3ccc4cccc5c4c3-c3ccccc3-5)cc2)cc1 ; CC1(C)c2ccccc2-c2ccc(N(c3ccccc3)c3cccc(-c4ccc5c6ccccc6c6cc7c8ccccc8n(-c8ccc9ccccc9c8)c7cc6c5c4)c3)cc21 ; c1ccc(-c2ccc(-n3c4ccccc4c4cc5c6ccccc6c6ccc(-c7ccc(N(c8ccc9ccccc9c8)c8ccc9c(c8)oc8ccccc89)cc7)cc6c5cc43)cc2)cc1]

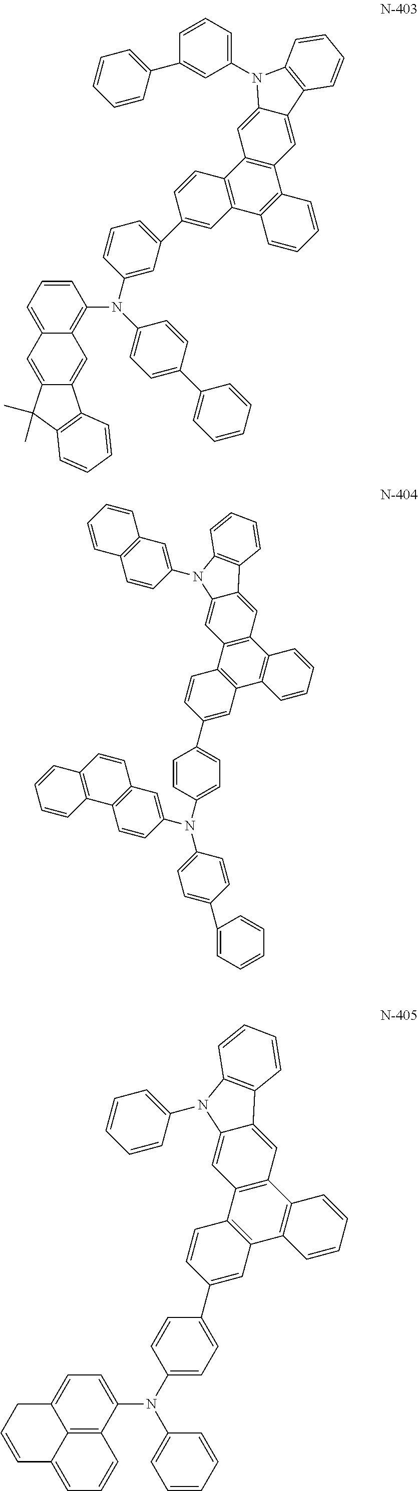

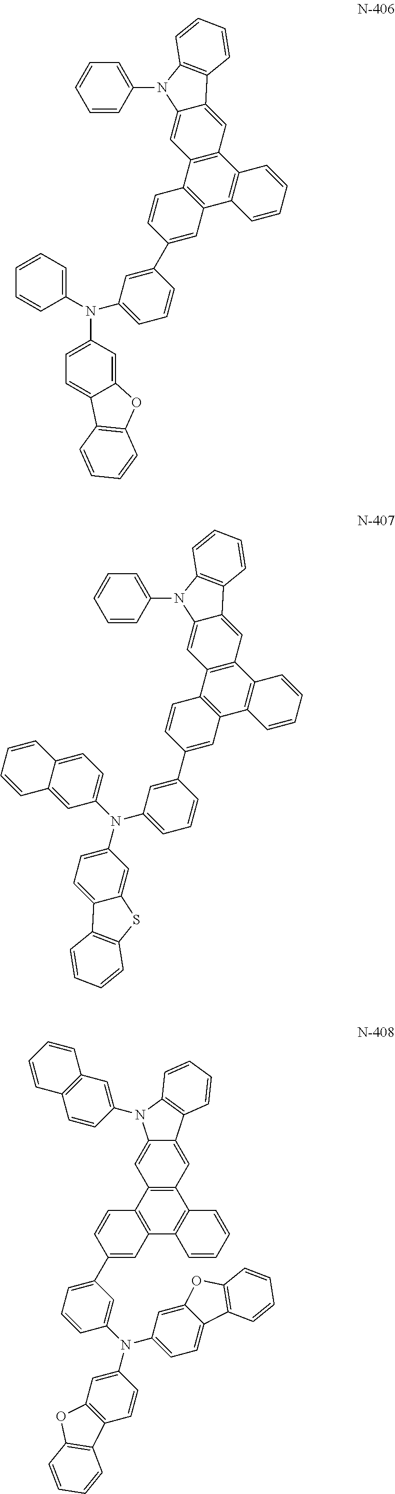 [SMILES: c1ccc(N(c2cccc(-c3ccc4c(c3)c3ccccc3c3cc5c6ccccc6n(-c6ccccc6)c5cc43)c2)c2ccc3c(c2)oc2ccccc23)cc1 ; c1ccc(-n2c3ccccc3c3cc4c5ccccc5c5cc(-c6cccc(N(c7ccc8ccccc8c7)c7ccc8c(c7)sc7ccccc78)c6)ccc5c4cc32)cc1 ; c1cc(-c2ccc3c(c2)c2ccccc2c2cc4c5ccccc5n(-c5ccc6ccccc6c5)c4cc32)cc(N(c2ccc3c(c2)oc2ccccc23)c2ccc3c(c2)oc2ccccc23)c1]

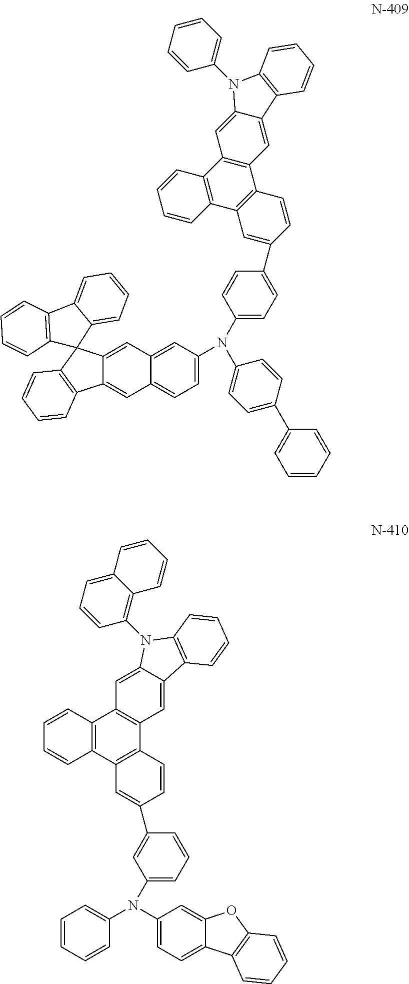 [SMILES: c1ccc(-c2ccc(N(c3ccc(-c4ccc5c(c4)c4ccccc4c4cc6c(cc54)c4ccccc4n6-c4ccccc4)cc3)c3ccc4cc5c(cc4c3)C3(c4ccccc4-c4ccccc43)c3ccccc3-5)cc2)cc1 ; c1ccc(N(c2cccc(-c3ccc4c(c3)c3ccccc3c3cc5c(cc43)c3ccccc3n5-c3cccc4ccccc34)c2)c2ccc3c(c2)oc2ccccc23)cc1]

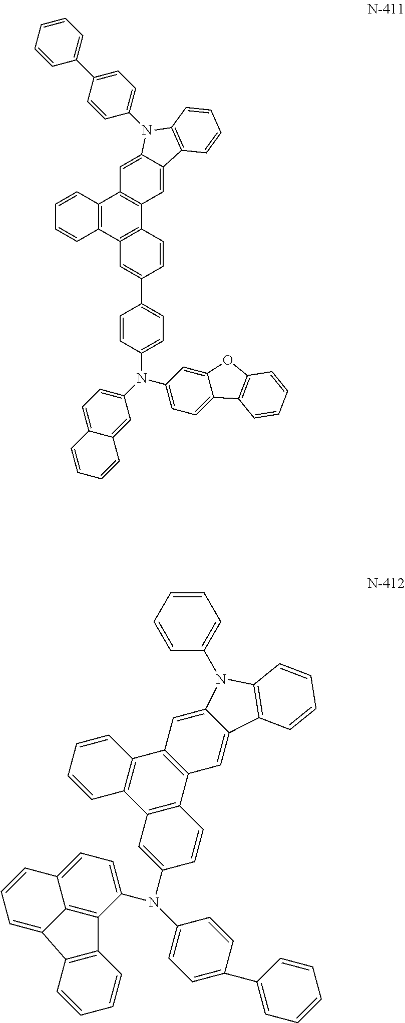 [SMILES: c1ccc(-c2ccc(-n3c4ccccc4c4cc5c6ccc(-c7ccc(N(c8ccc9ccccc9c8)c8ccc9c(c8)oc8ccccc89)cc7)cc6c6ccccc6c5cc43)cc2)cc1 ; c1ccc(-c2ccc(N(c3ccc4c(c3)c3ccccc3c3cc5c(cc43)c3ccccc3n5-c3ccccc3)c3ccc4cccc5c4c3-c3ccccc3-5)cc2)cc1]

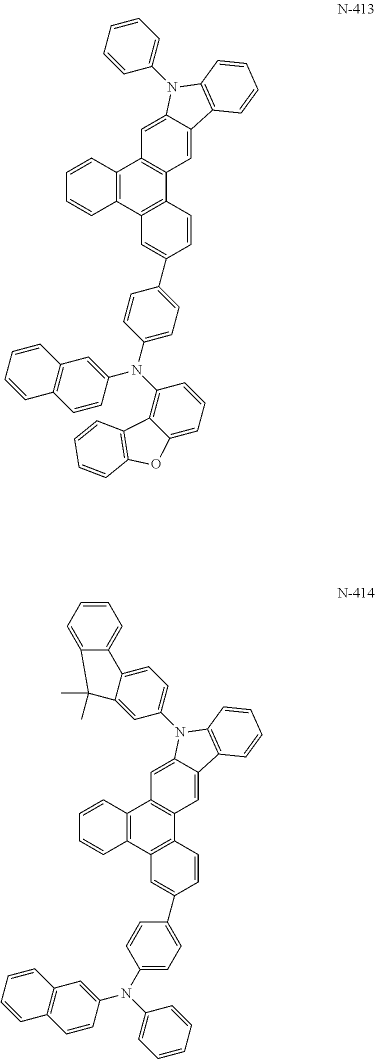 [SMILES: c1ccc(-n2c3ccccc3c3cc4c5ccc(-c6ccc(N(c7ccc8ccccc8c7)c7cccc8oc9ccccc9c78)cc6)cc5c5ccccc5c4cc32)cc1 ; CC1(C)c2ccccc2-c2ccc(-n3c4ccccc4c4cc5c6ccc(-c7ccc(N(c8ccccc8)c8ccc9ccccc9c8)cc7)cc6c6ccccc6c5cc43)cc21]

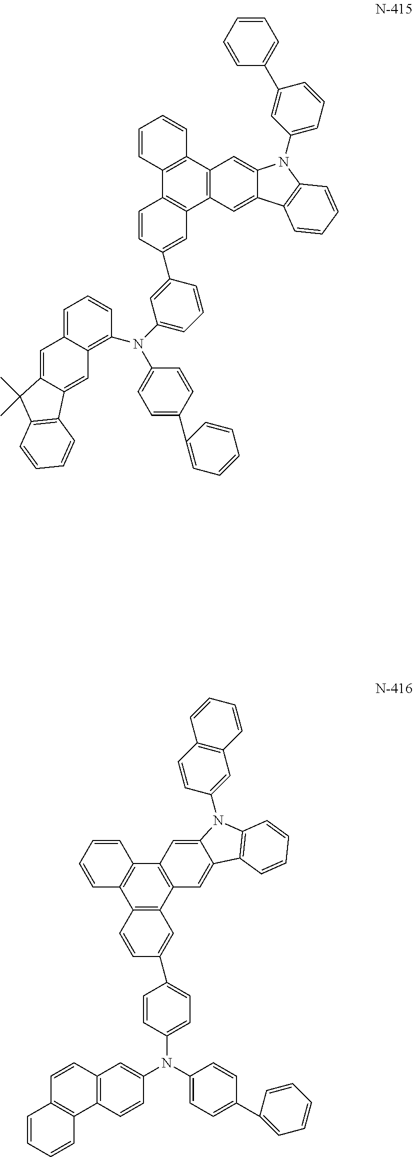 [SMILES: CC1(C)c2ccccc2-c2cc3c(N(c4ccc(-c5ccccc5)cc4)c4cccc(-c5ccc6c7ccccc7c7cc8c(cc7c6c5)c5ccccc5n8-c5cccc(-c6ccccc6)c5)c4)cccc3cc21 ; c1ccc(-c2ccc(N(c3ccc(-c4ccc5c6ccccc6c6cc7c(cc6c5c4)c4ccccc4n7-c4ccc5ccccc5c4)cc3)c3ccc4c(ccc5ccccc54)c3)cc2)cc1]

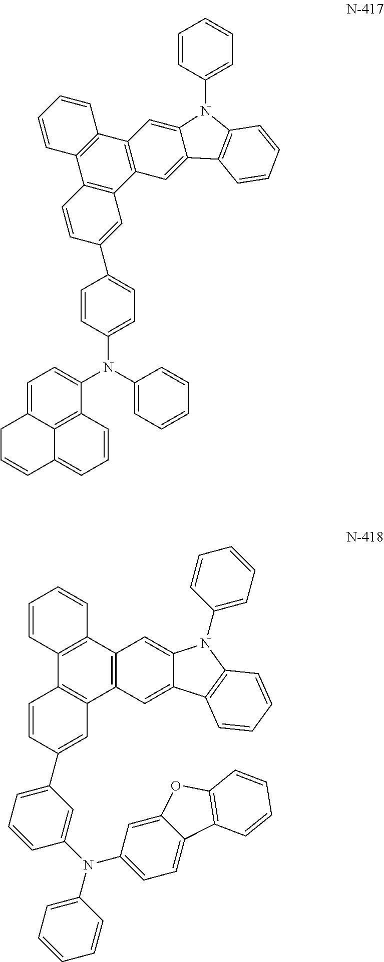 [SMILES: C1=Cc2cccc3c(N(c4ccccc4)c4ccc(-c5ccc6c7ccccc7c7cc8c(cc7c6c5)c5ccccc5n8-c5ccccc5)cc4)ccc(c23)C1 ; c1ccc(N(c2cccc(-c3ccc4c5ccccc5c5cc6c(cc5c4c3)c3ccccc3n6-c3ccccc3)c2)c2ccc3c(c2)oc2ccccc23)cc1]

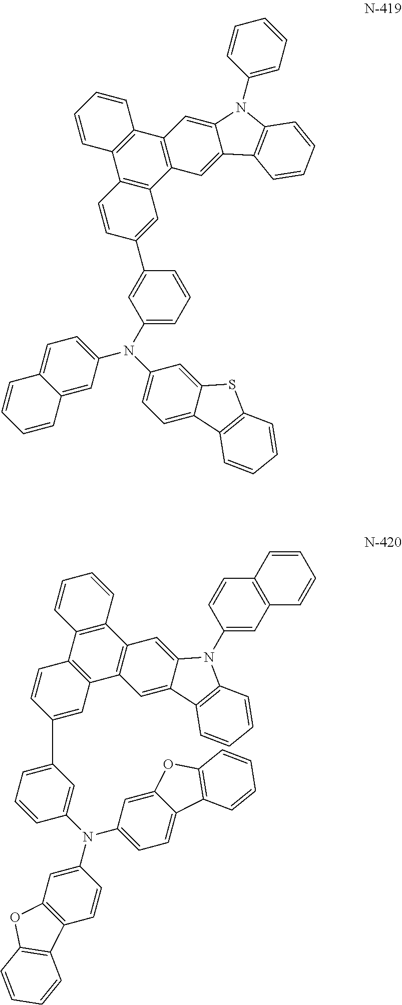 [SMILES: c1ccc(-n2c3ccccc3c3cc4c5cc(-c6cccc(N(c7ccc8ccccc8c7)c7ccc8c(c7)sc7ccccc78)c6)ccc5c5ccccc5c4cc32)cc1 ; c1cc(-c2ccc3c4ccccc4c4cc5c(cc4c3c2)c2ccccc2n5-c2ccc3ccccc3c2)cc(N(c2ccc3c(c2)oc2ccccc23)c2ccc3c(c2)oc2ccccc23)c1]

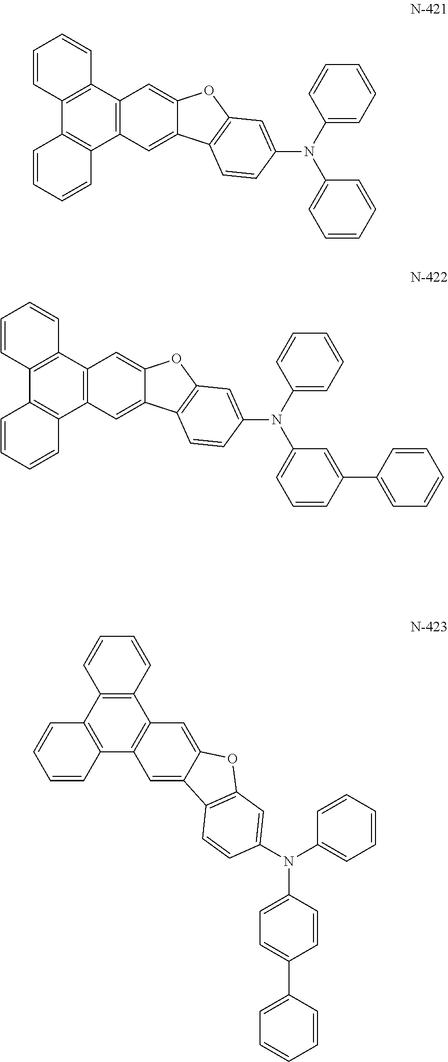 [SMILES: c1ccc(N(c2ccccc2)c2ccc3c(c2)oc2cc4c5ccccc5c5ccccc5c4cc23)cc1 ; c1ccc(-c2cccc(N(c3ccccc3)c3ccc4c(c3)oc3cc5c6ccccc6c6ccccc6c5cc34)c2)cc1 ; c1ccc(-c2ccc(N(c3ccccc3)c3ccc4c(c3)oc3cc5c6ccccc6c6ccccc6c5cc34)cc2)cc1]

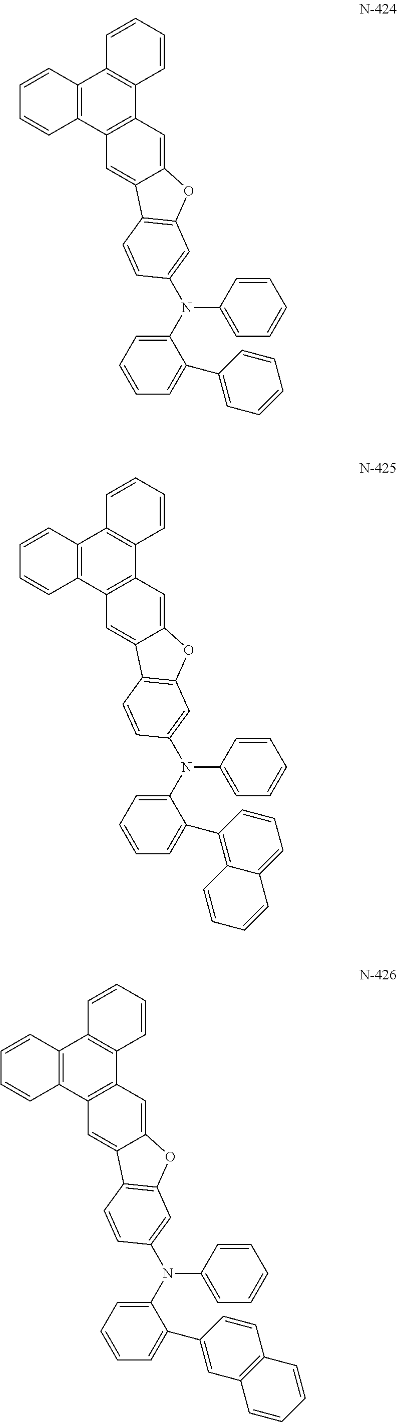 [SMILES: c1ccc(-c2ccccc2N(c2ccccc2)c2ccc3c(c2)oc2cc4c5ccccc5c5ccccc5c4cc23)cc1 ; c1ccc(N(c2ccc3c(c2)oc2cc4c5ccccc5c5ccccc5c4cc23)c2ccccc2-c2cccc3ccccc23)cc1 ; c1ccc(N(c2ccc3c(c2)oc2cc4c5ccccc5c5ccccc5c4cc23)c2ccccc2-c2ccc3ccccc3c2)cc1]

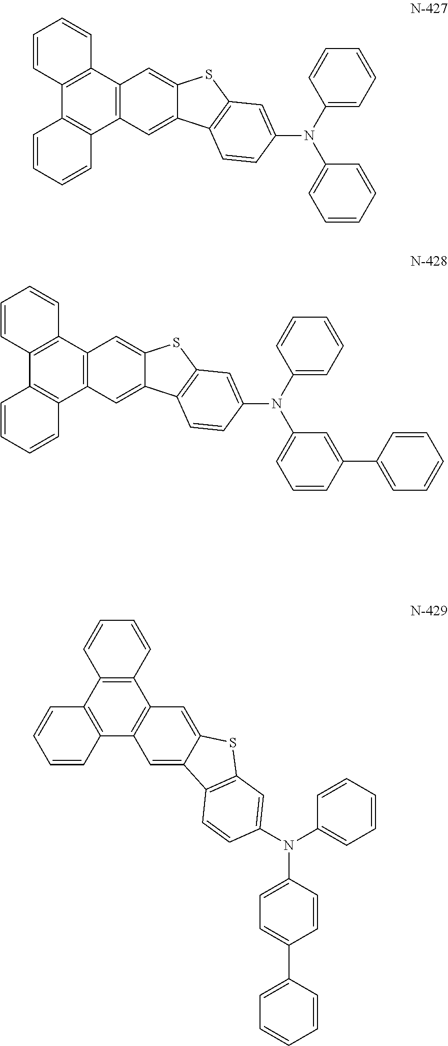 [SMILES: c1ccc(N(c2ccccc2)c2ccc3c(c2)sc2cc4c5ccccc5c5ccccc5c4cc23)cc1 ; c1ccc(-c2cccc(N(c3ccccc3)c3ccc4c(c3)sc3cc5c6ccccc6c6ccccc6c5cc34)c2)cc1 ; c1ccc(-c2ccc(N(c3ccccc3)c3ccc4c(c3)sc3cc5c6ccccc6c6ccccc6c5cc34)cc2)cc1]

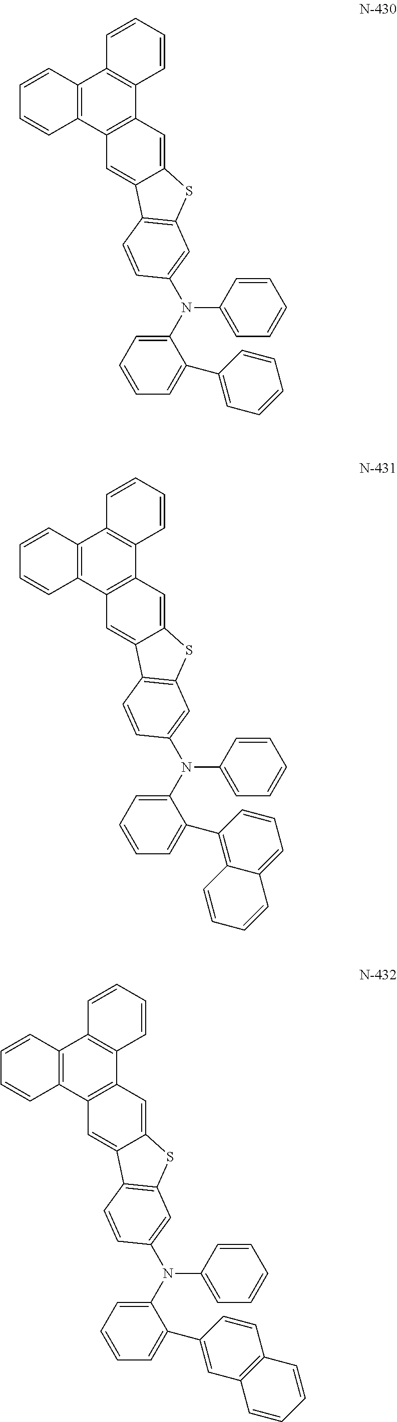 [SMILES: c1ccc(-c2ccccc2N(c2ccccc2)c2ccc3c(c2)sc2cc4c5ccccc5c5ccccc5c4cc23)cc1 ; c1ccc(N(c2ccc3c(c2)sc2cc4c5ccccc5c5ccccc5c4cc23)c2ccccc2-c2cccc3ccccc23)cc1 ; c1ccc(N(c2ccc3c(c2)sc2cc4c5ccccc5c5ccccc5c4cc23)c2ccccc2-c2ccc3ccccc3c2)cc1]

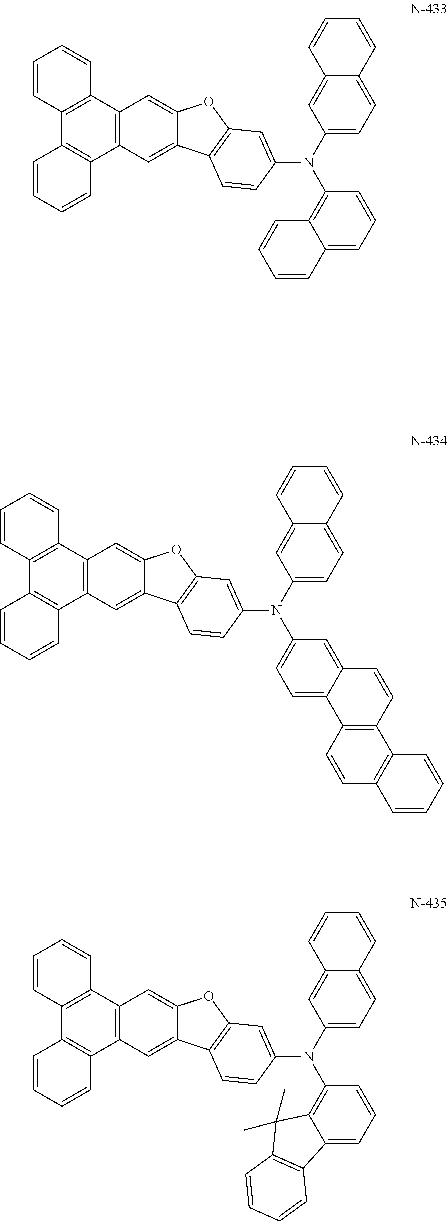 [SMILES: c1ccc2cc(N(c3ccc4c(c3)oc3cc5c6ccccc6c6ccccc6c5cc34)c3cccc4ccccc34)ccc2c1 ; c1ccc2cc(N(c3ccc4c(ccc5c6ccccc6ccc45)c3)c3ccc4c(c3)oc3cc5c6ccccc6c6ccccc6c5cc34)ccc2c1 ; CC1(C)c2ccccc2-c2cccc(N(c3ccc4ccccc4c3)c3ccc4c(c3)oc3cc5c6ccccc6c6ccccc6c5cc34)c21]

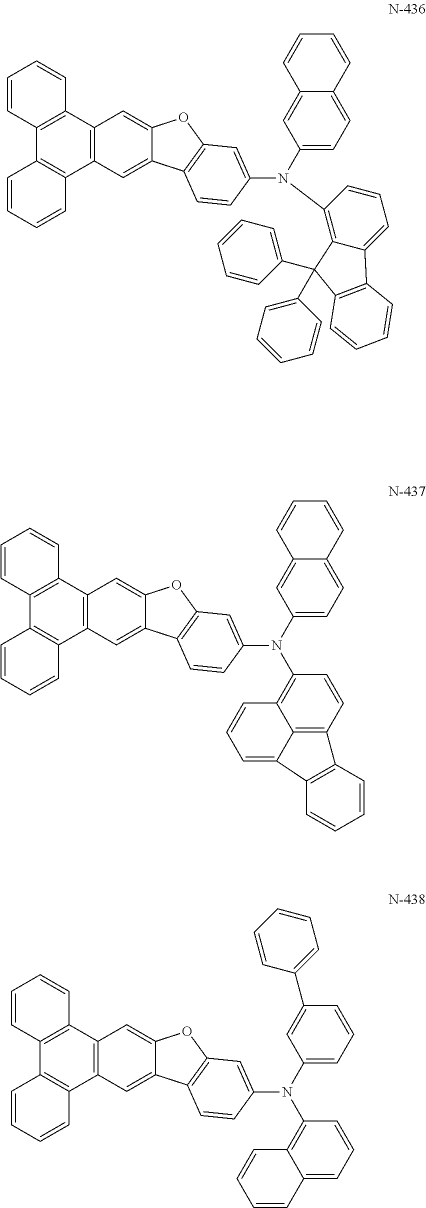 [SMILES: c1ccc(C2(c3ccccc3)c3ccccc3-c3cccc(N(c4ccc5ccccc5c4)c4ccc5c(c4)oc4cc6c7ccccc7c7ccccc7c6cc45)c32)cc1 ; c1ccc2c(c1)-c1cccc3c(N(c4ccc5ccccc5c4)c4ccc5c(c4)oc4cc6c7ccccc7c7ccccc7c6cc45)ccc-2c13 ; c1ccc(-c2cccc(N(c3ccc4c(c3)oc3cc5c6ccccc6c6ccccc6c5cc34)c3cccc4ccccc34)c2)cc1]

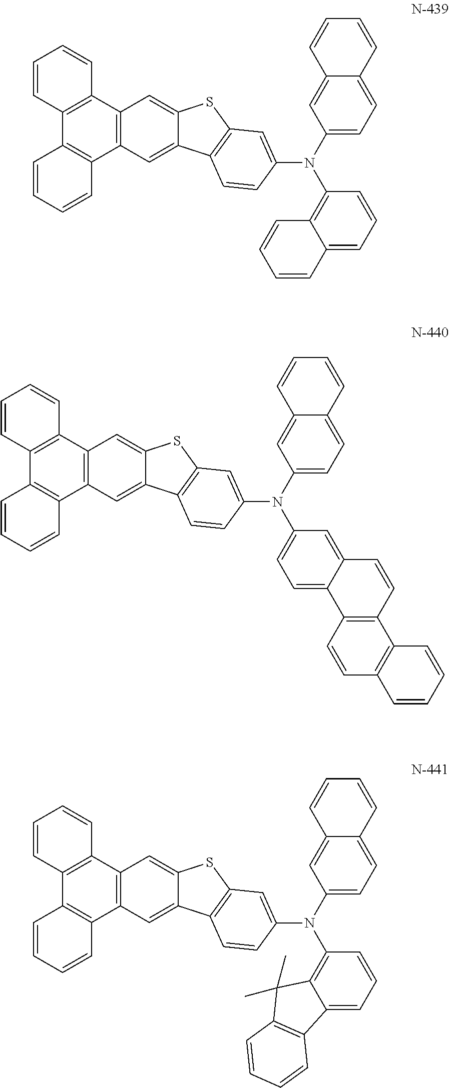 [SMILES: c1ccc2cc(N(c3ccc4c(c3)sc3cc5c6ccccc6c6ccccc6c5cc34)c3cccc4ccccc34)ccc2c1 ; c1ccc2cc(N(c3ccc4c(ccc5c6ccccc6ccc45)c3)c3ccc4c(c3)sc3cc5c6ccccc6c6ccccc6c5cc34)ccc2c1 ; CC1(C)c2ccccc2-c2cccc(N(c3ccc4ccccc4c3)c3ccc4c(c3)sc3cc5c6ccccc6c6ccccc6c5cc34)c21]

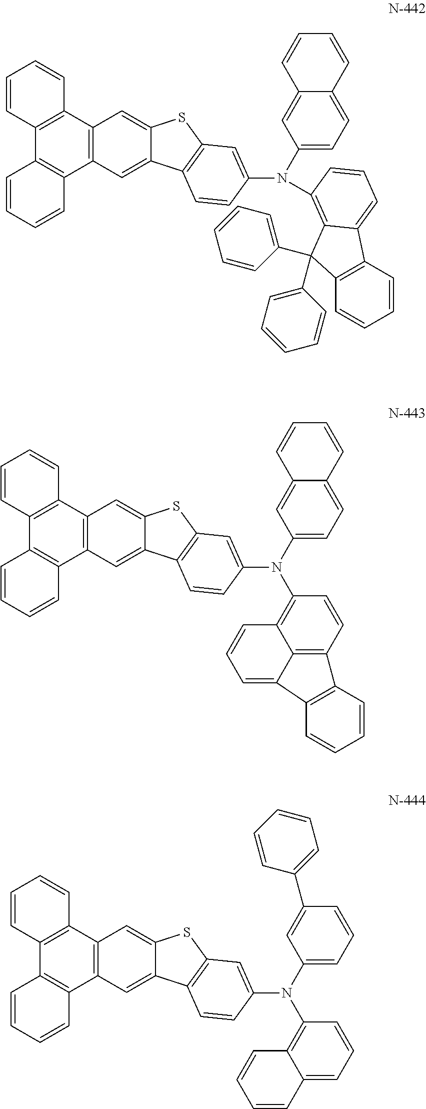 [SMILES: c1ccc(C2(c3ccccc3)c3ccccc3-c3cccc(N(c4ccc5ccccc5c4)c4ccc5c(c4)sc4cc6c7ccccc7c7ccccc7c6cc45)c32)cc1 ; c1ccc2c(c1)-c1cccc3c(N(c4ccc5ccccc5c4)c4ccc5c(c4)sc4cc6c7ccccc7c7ccccc7c6cc45)ccc-2c13 ; c1ccc(-c2cccc(N(c3ccc4c(c3)sc3cc5c6ccccc6c6ccccc6c5cc34)c3cccc4ccccc34)c2)cc1]

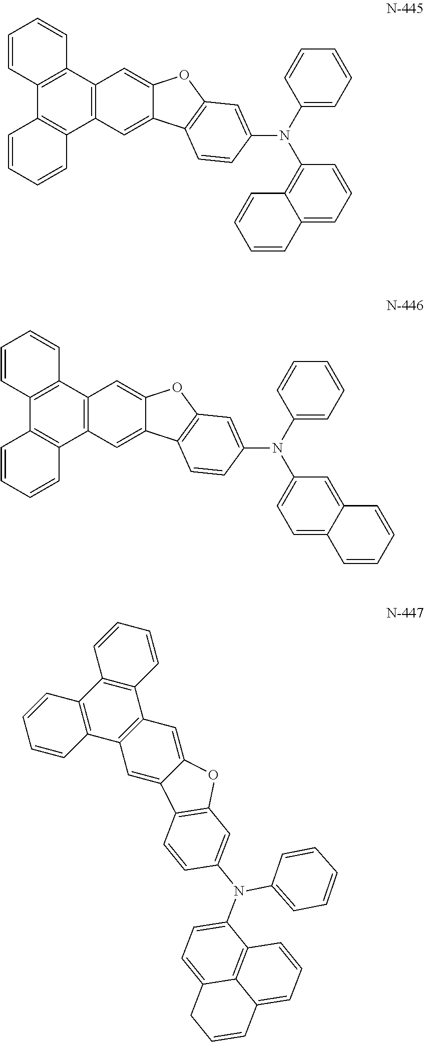 [SMILES: c1ccc(N(c2ccc3c(c2)oc2cc4c5ccccc5c5ccccc5c4cc23)c2cccc3ccccc23)cc1 ; c1ccc(N(c2ccc3ccccc3c2)c2ccc3c(c2)oc2cc4c5ccccc5c5ccccc5c4cc23)cc1 ; C1=Cc2cccc3c(N(c4ccccc4)c4ccc5c(c4)oc4cc6c7ccccc7c7ccccc7c6cc45)ccc(c23)C1]

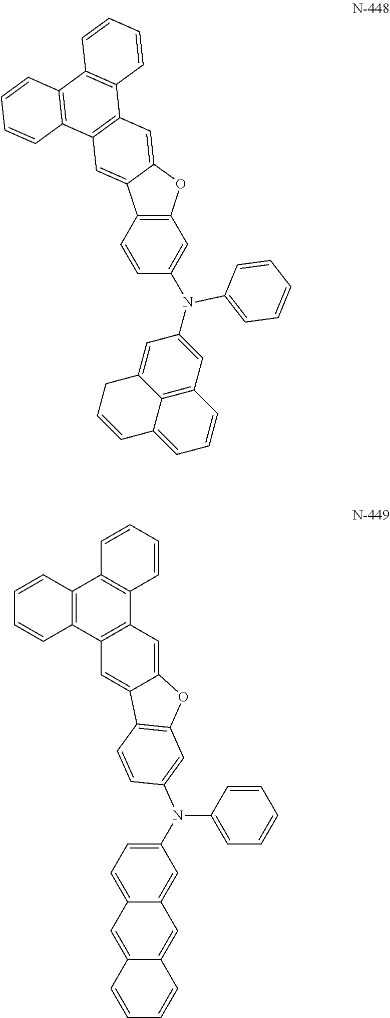 [SMILES: C1=Cc2cccc3cc(N(c4ccccc4)c4ccc5c(c4)oc4cc6c7ccccc7c7ccccc7c6cc45)cc(c23)C1 ; c1ccc(N(c2ccc3cc4ccccc4cc3c2)c2ccc3c(c2)oc2cc4c5ccccc5c5ccccc5c4cc23)cc1]

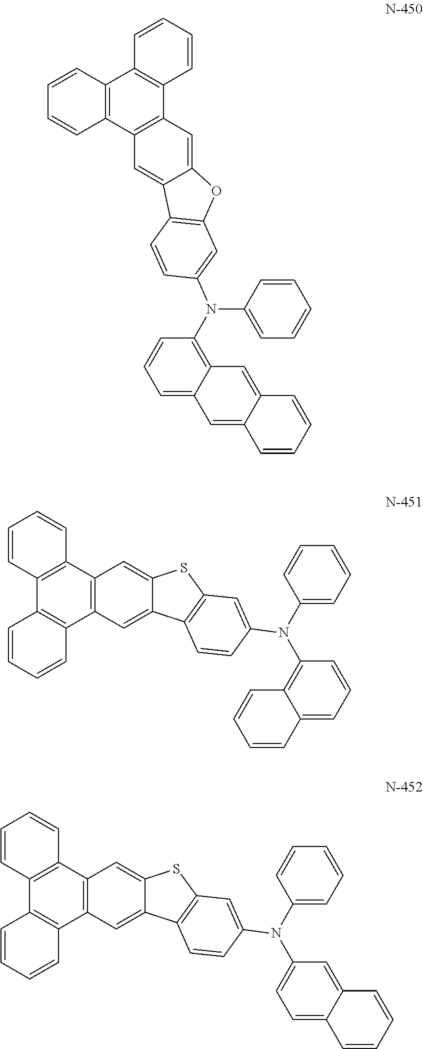 [SMILES: c1ccc(N(c2ccc3c(c2)oc2cc4c5ccccc5c5ccccc5c4cc23)c2cccc3cc4ccccc4cc23)cc1 ; c1ccc(N(c2ccc3c(c2)sc2cc4c5ccccc5c5ccccc5c4cc23)c2cccc3ccccc23)cc1 ; c1ccc(N(c2ccc3ccccc3c2)c2ccc3c(c2)sc2cc4c5ccccc5c5ccccc5c4cc23)cc1]

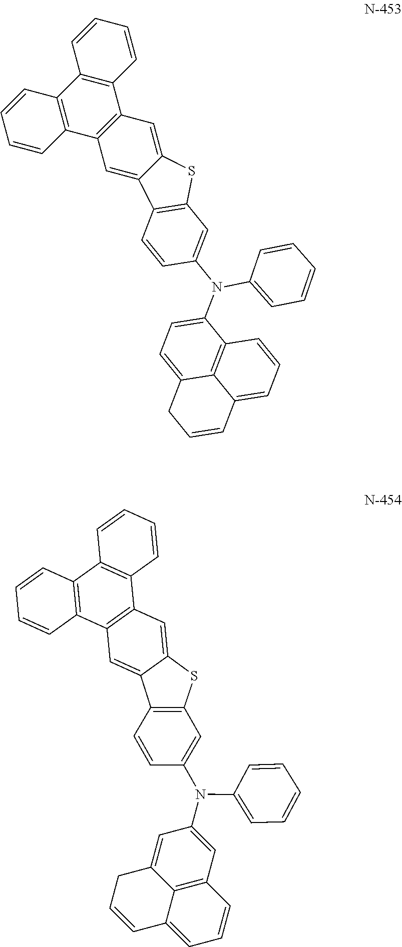 [SMILES: C1=Cc2cccc3c(N(c4ccccc4)c4ccc5c(c4)sc4cc6c7ccccc7c7ccccc7c6cc45)ccc(c23)C1 ; C1=Cc2cccc3cc(N(c4ccccc4)c4ccc5c(c4)sc4cc6c7ccccc7c7ccccc7c6cc45)cc(c23)C1]

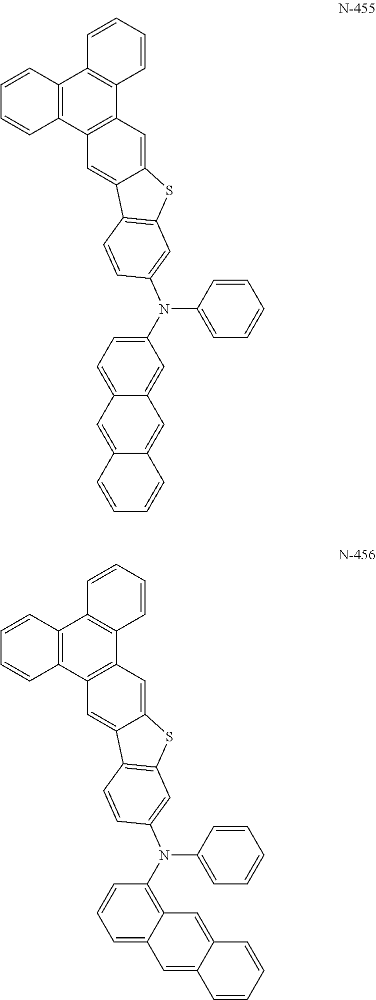 [SMILES: c1ccc(N(c2ccc3cc4ccccc4cc3c2)c2ccc3c(c2)sc2cc4c5ccccc5c5ccccc5c4cc23)cc1 ; c1ccc(N(c2ccc3c(c2)sc2cc4c5ccccc5c5ccccc5c4cc23)c2cccc3cc4ccccc4cc23)cc1]

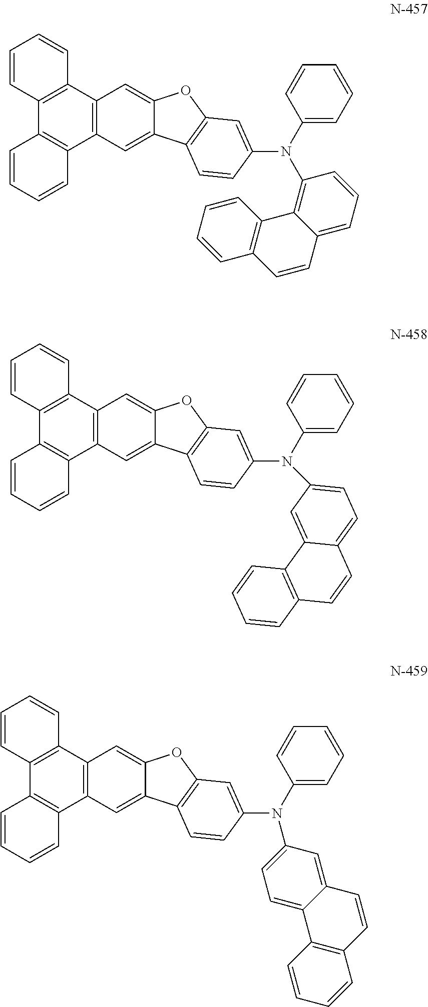 [SMILES: c1ccc(N(c2ccc3c(c2)oc2cc4c5ccccc5c5ccccc5c4cc23)c2cccc3ccc4ccccc4c23)cc1 ; c1ccc(N(c2ccc3c(c2)oc2cc4c5ccccc5c5ccccc5c4cc23)c2ccc3ccc4ccccc4c3c2)cc1 ; c1ccc(N(c2ccc3c(ccc4ccccc43)c2)c2ccc3c(c2)oc2cc4c5ccccc5c5ccccc5c4cc23)cc1]

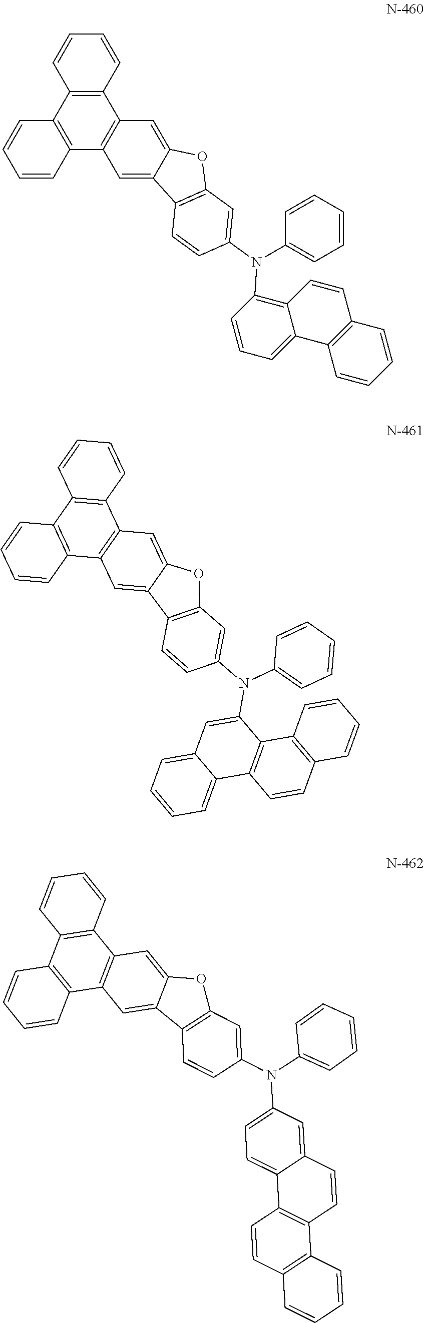 [SMILES: c1ccc(N(c2ccc3c(c2)oc2cc4c5ccccc5c5ccccc5c4cc23)c2cccc3c2ccc2ccccc23)cc1 ; c1ccc(N(c2ccc3c(c2)oc2cc4c5ccccc5c5ccccc5c4cc23)c2cc3ccccc3c3ccc4ccccc4c23)cc1 ; c1ccc(N(c2ccc3c(ccc4c5ccccc5ccc34)c2)c2ccc3c(c2)oc2cc4c5ccccc5c5ccccc5c4cc23)cc1]

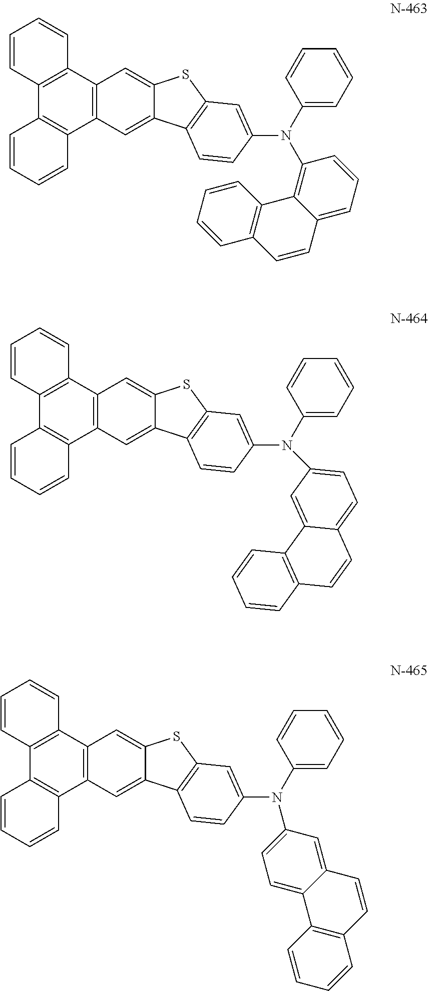 [SMILES: c1ccc(N(c2ccc3c(c2)sc2cc4c5ccccc5c5ccccc5c4cc23)c2cccc3ccc4ccccc4c23)cc1 ; c1ccc(N(c2ccc3c(c2)sc2cc4c5ccccc5c5ccccc5c4cc23)c2ccc3ccc4ccccc4c3c2)cc1 ; c1ccc(N(c2ccc3c(ccc4ccccc43)c2)c2ccc3c(c2)sc2cc4c5ccccc5c5ccccc5c4cc23)cc1]

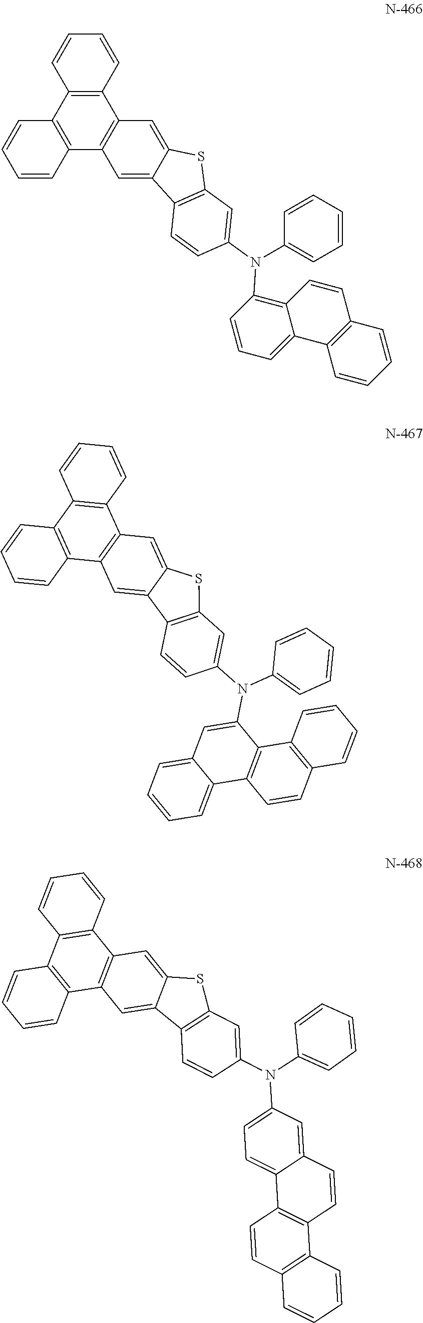 [SMILES: c1ccc(N(c2ccc3c(c2)sc2cc4c5ccccc5c5ccccc5c4cc23)c2cccc3c2ccc2ccccc23)cc1 ; c1ccc(N(c2ccc3c(c2)sc2cc4c5ccccc5c5ccccc5c4cc23)c2cc3ccccc3c3ccc4ccccc4c23)cc1 ; c1ccc(N(c2ccc3c(ccc4c5ccccc5ccc34)c2)c2ccc3c(c2)sc2cc4c5ccccc5c5ccccc5c4cc23)cc1]

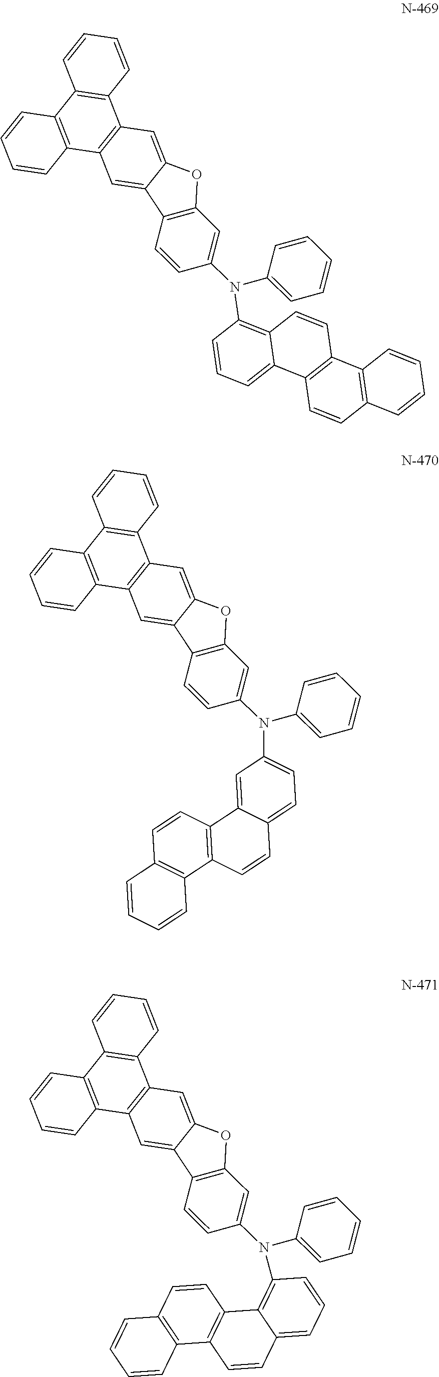 [SMILES: c1ccc(N(c2ccc3c(c2)oc2cc4c5ccccc5c5ccccc5c4cc23)c2cccc3c2ccc2c4ccccc4ccc32)cc1 ; c1ccc(N(c2ccc3c(c2)oc2cc4c5ccccc5c5ccccc5c4cc23)c2ccc3ccc4c5ccccc5ccc4c3c2)cc1 ; c1ccc(N(c2ccc3c(c2)oc2cc4c5ccccc5c5ccccc5c4cc23)c2cccc3ccc4c5ccccc5ccc4c23)cc1]

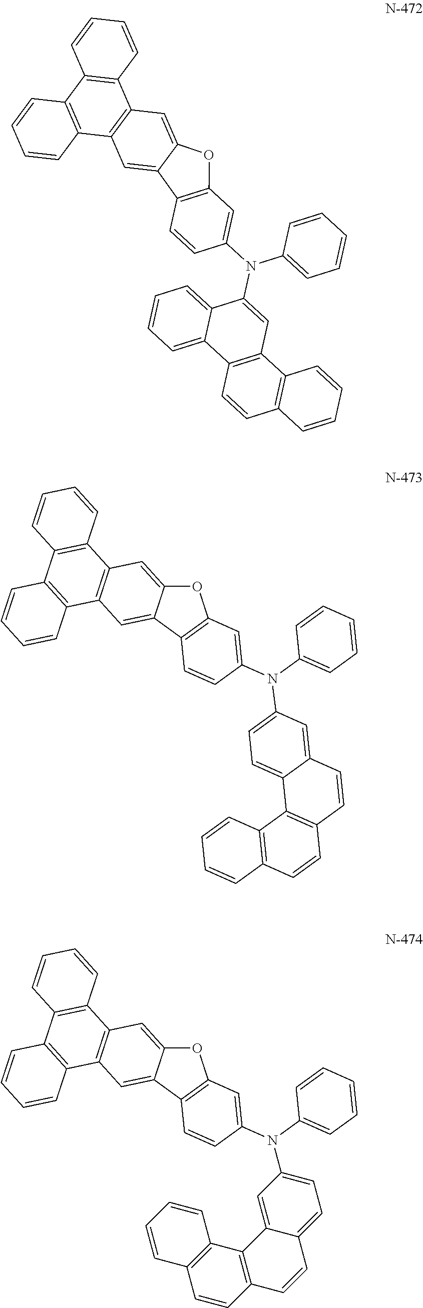 [SMILES: c1ccc(N(c2ccc3c(c2)oc2cc4c5ccccc5c5ccccc5c4cc23)c2cc3c4ccccc4ccc3c3ccccc23)cc1 ; c1ccc(N(c2ccc3c(ccc4ccc5ccccc5c43)c2)c2ccc3c(c2)oc2cc4c5ccccc5c5ccccc5c4cc23)cc1 ; c1ccc(N(c2ccc3c(c2)oc2cc4c5ccccc5c5ccccc5c4cc23)c2ccc3ccc4ccc5ccccc5c4c3c2)cc1]

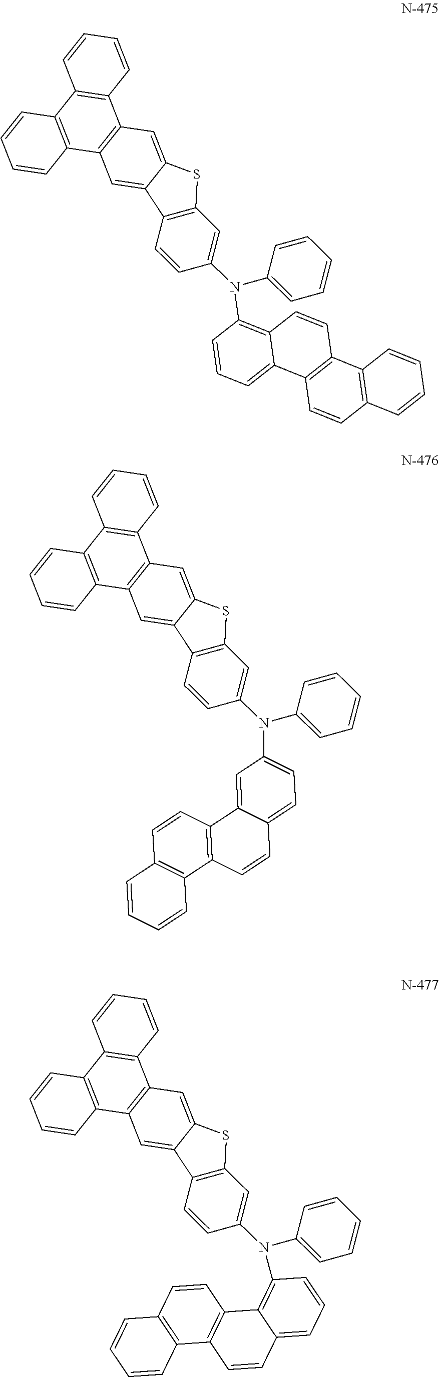 [SMILES: c1ccc(N(c2ccc3c(c2)sc2cc4c5ccccc5c5ccccc5c4cc23)c2cccc3c2ccc2c4ccccc4ccc32)cc1 ; c1ccc(N(c2ccc3c(c2)sc2cc4c5ccccc5c5ccccc5c4cc23)c2ccc3ccc4c5ccccc5ccc4c3c2)cc1 ; c1ccc(N(c2ccc3c(c2)sc2cc4c5ccccc5c5ccccc5c4cc23)c2cccc3ccc4c5ccccc5ccc4c23)cc1]

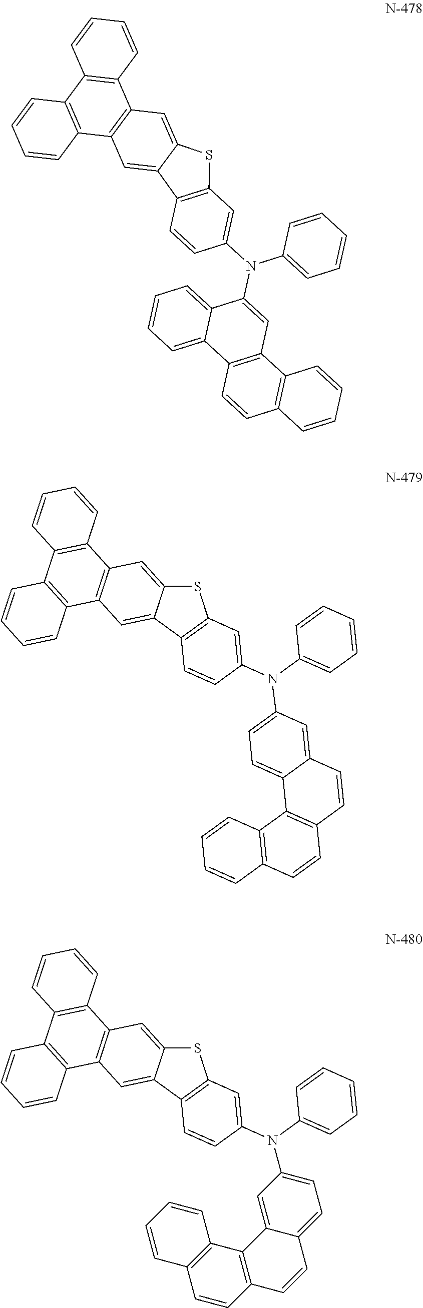 [SMILES: c1ccc(N(c2ccc3c(c2)sc2cc4c5ccccc5c5ccccc5c4cc23)c2cc3c4ccccc4ccc3c3ccccc23)cc1 ; c1ccc(N(c2ccc3c(ccc4ccc5ccccc5c43)c2)c2ccc3c(c2)sc2cc4c5ccccc5c5ccccc5c4cc23)cc1 ; c1ccc(N(c2ccc3c(c2)sc2cc4c5ccccc5c5ccccc5c4cc23)c2ccc3ccc4ccc5ccccc5c4c3c2)cc1]

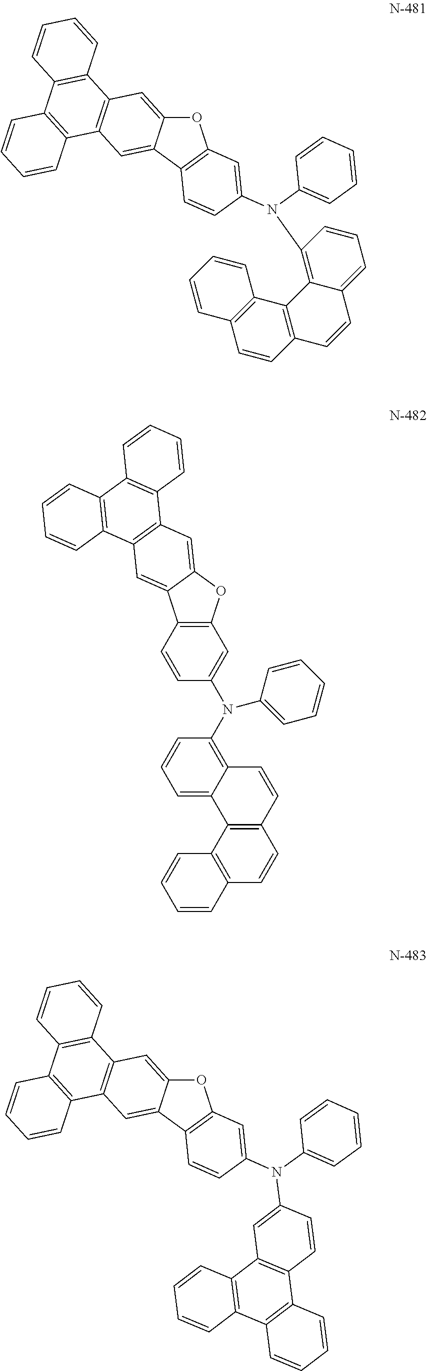 [SMILES: c1ccc(N(c2ccc3c(c2)oc2cc4c5ccccc5c5ccccc5c4cc23)c2cccc3ccc4ccc5ccccc5c4c23)cc1 ; c1ccc(N(c2ccc3c(c2)oc2cc4c5ccccc5c5ccccc5c4cc23)c2cccc3c2ccc2ccc4ccccc4c23)cc1 ; c1ccc(N(c2ccc3c(c2)oc2cc4c5ccccc5c5ccccc5c4cc23)c2ccc3c4ccccc4c4ccccc4c3c2)cc1]

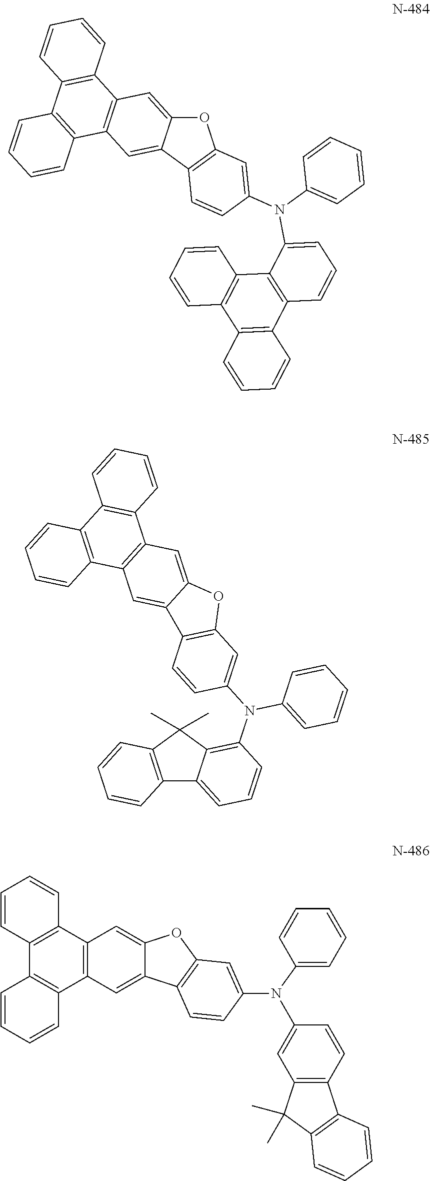 [SMILES: c1ccc(N(c2ccc3c(c2)oc2cc4c5ccccc5c5ccccc5c4cc23)c2cccc3c4ccccc4c4ccccc4c23)cc1 ; CC1(C)c2ccccc2-c2cccc(N(c3ccccc3)c3ccc4c(c3)oc3cc5c6ccccc6c6ccccc6c5cc34)c21 ; CC1(C)c2ccccc2-c2ccc(N(c3ccccc3)c3ccc4c(c3)oc3cc5c6ccccc6c6ccccc6c5cc34)cc21]

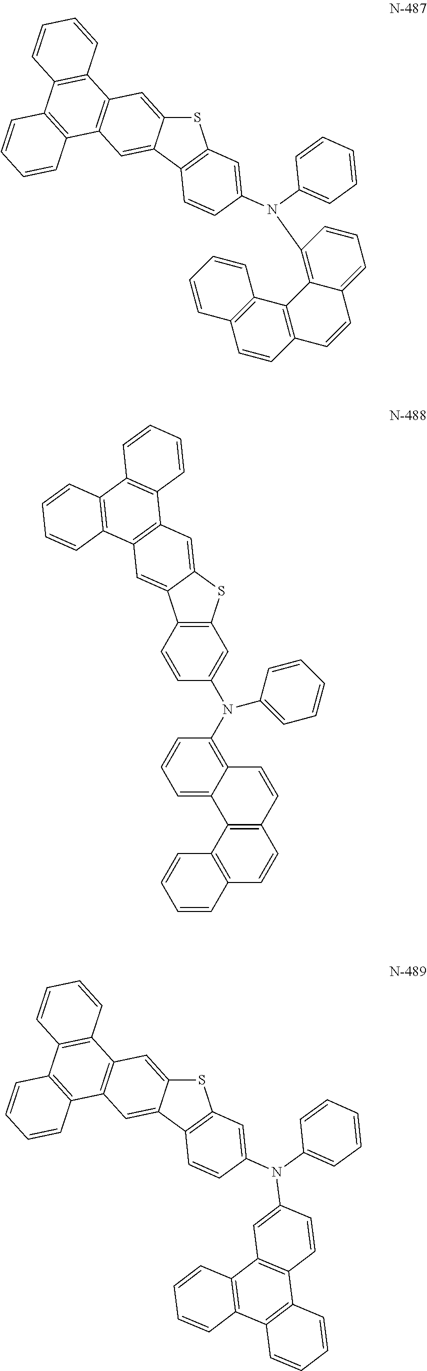 [SMILES: c1ccc(N(c2ccc3c(c2)sc2cc4c5ccccc5c5ccccc5c4cc23)c2cccc3ccc4ccc5ccccc5c4c23)cc1 ; c1ccc(N(c2ccc3c(c2)sc2cc4c5ccccc5c5ccccc5c4cc23)c2cccc3c2ccc2ccc4ccccc4c23)cc1 ; c1ccc(N(c2ccc3c(c2)sc2cc4c5ccccc5c5ccccc5c4cc23)c2ccc3c4ccccc4c4ccccc4c3c2)cc1]

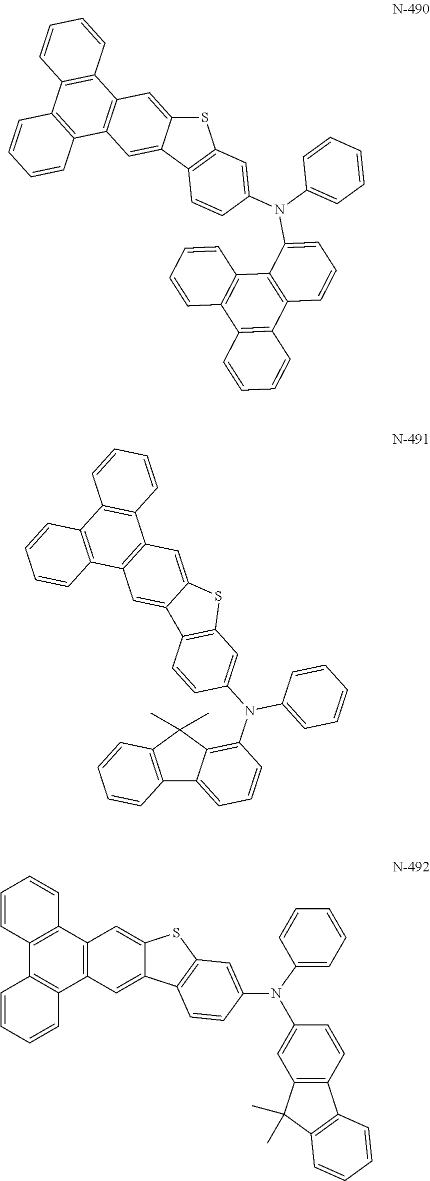 [SMILES: c1ccc(N(c2ccc3c(c2)sc2cc4c5ccccc5c5ccccc5c4cc23)c2cccc3c4ccccc4c4ccccc4c23)cc1 ; CC1(C)c2ccccc2-c2cccc(N(c3ccccc3)c3ccc4c(c3)sc3cc5c6ccccc6c6ccccc6c5cc34)c21 ; CC1(C)c2ccccc2-c2ccc(N(c3ccccc3)c3ccc4c(c3)sc3cc5c6ccccc6c6ccccc6c5cc34)cc21]

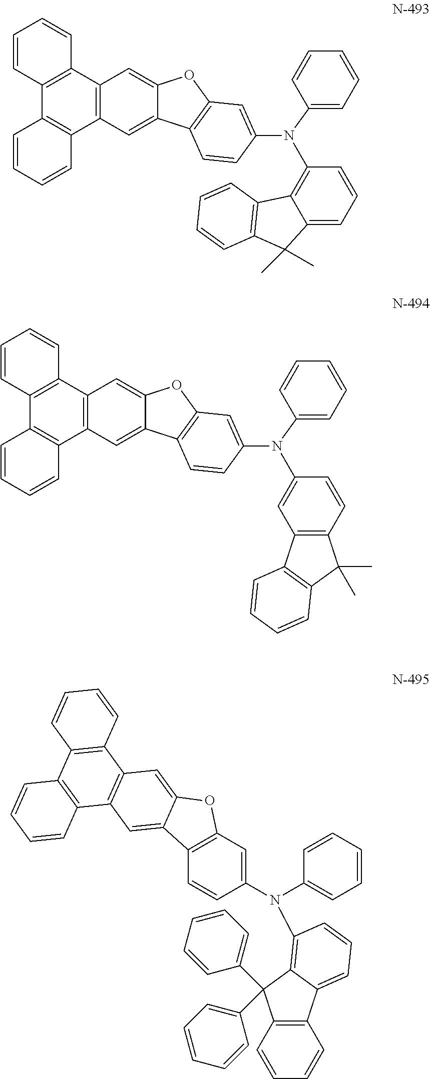 [SMILES: CC1(C)c2ccccc2-c2c(N(c3ccccc3)c3ccc4c(c3)oc3cc5c6ccccc6c6ccccc6c5cc34)cccc21 ; CC1(C)c2ccccc2-c2cc(N(c3ccccc3)c3ccc4c(c3)oc3cc5c6ccccc6c6ccccc6c5cc34)ccc21 ; c1ccc(N(c2ccc3c(c2)oc2cc4c5ccccc5c5ccccc5c4cc23)c2cccc3c2C(c2ccccc2)(c2ccccc2)c2ccccc2-3)cc1]

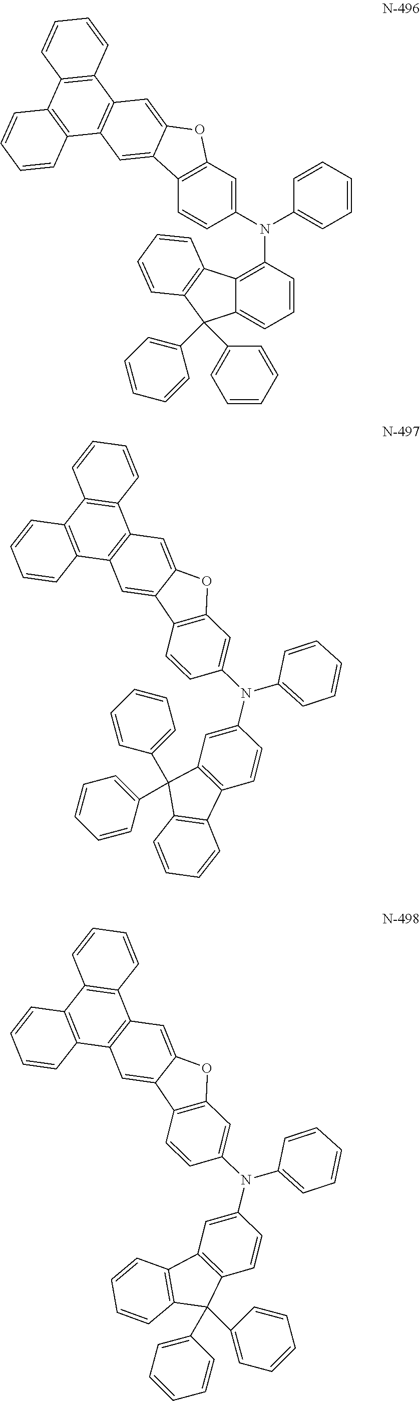 [SMILES: c1ccc(N(c2ccc3c(c2)oc2cc4c5ccccc5c5ccccc5c4cc23)c2cccc3c2-c2ccccc2C3(c2ccccc2)c2ccccc2)cc1 ; c1ccc(N(c2ccc3c(c2)C(c2ccccc2)(c2ccccc2)c2ccccc2-3)c2ccc3c(c2)oc2cc4c5ccccc5c5ccccc5c4cc23)cc1 ; c1ccc(N(c2ccc3c(c2)-c2ccccc2C3(c2ccccc2)c2ccccc2)c2ccc3c(c2)oc2cc4c5ccccc5c5ccccc5c4cc23)cc1]

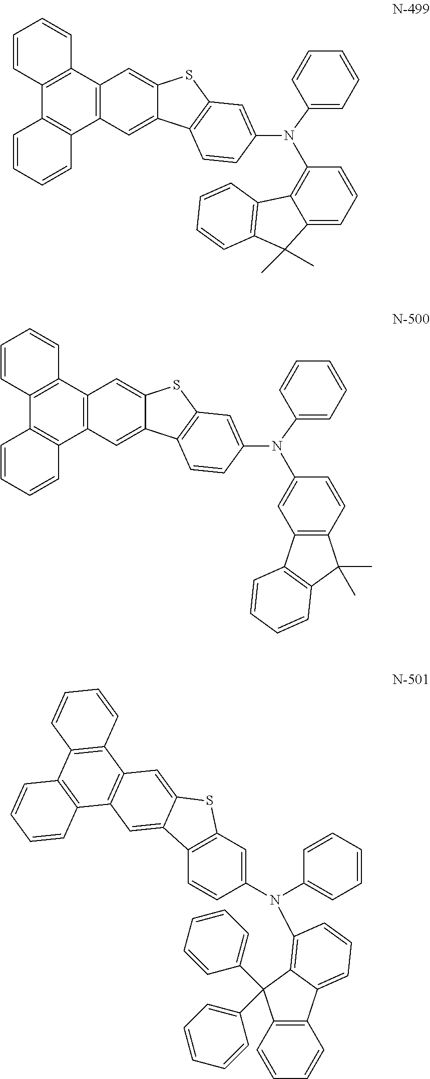 [SMILES: CC1(C)c2ccccc2-c2c(N(c3ccccc3)c3ccc4c(c3)sc3cc5c6ccccc6c6ccccc6c5cc34)cccc21 ; CC1(C)c2ccccc2-c2cc(N(c3ccccc3)c3ccc4c(c3)sc3cc5c6ccccc6c6ccccc6c5cc34)ccc21 ; c1ccc(N(c2ccc3c(c2)sc2cc4c5ccccc5c5ccccc5c4cc23)c2cccc3c2C(c2ccccc2)(c2ccccc2)c2ccccc2-3)cc1]

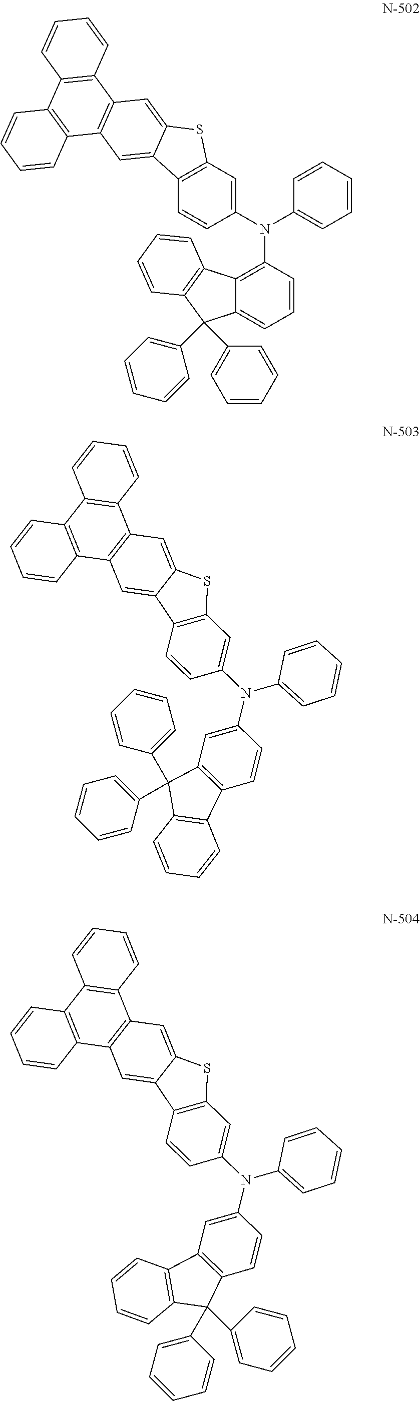 [SMILES: c1ccc(N(c2ccc3c(c2)sc2cc4c5ccccc5c5ccccc5c4cc23)c2cccc3c2-c2ccccc2C3(c2ccccc2)c2ccccc2)cc1 ; c1ccc(N(c2ccc3c(c2)C(c2ccccc2)(c2ccccc2)c2ccccc2-3)c2ccc3c(c2)sc2cc4c5ccccc5c5ccccc5c4cc23)cc1 ; c1ccc(N(c2ccc3c(c2)-c2ccccc2C3(c2ccccc2)c2ccccc2)c2ccc3c(c2)sc2cc4c5ccccc5c5ccccc5c4cc23)cc1]

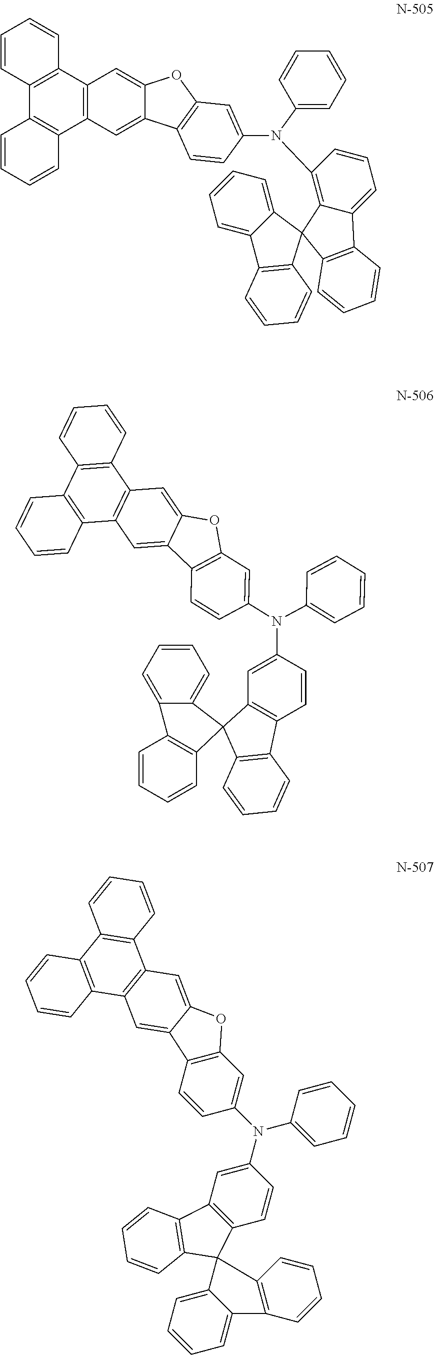 [SMILES: c1ccc(N(c2ccc3c(c2)oc2cc4c5ccccc5c5ccccc5c4cc23)c2cccc3c2C2(c4ccccc4-c4ccccc42)c2ccccc2-3)cc1 ; c1ccc(N(c2ccc3c(c2)C2(c4ccccc4-c4ccccc42)c2ccccc2-3)c2ccc3c(c2)oc2cc4c5ccccc5c5ccccc5c4cc23)cc1 ; c1ccc(N(c2ccc3c(c2)-c2ccccc2C32c3ccccc3-c3ccccc32)c2ccc3c(c2)oc2cc4c5ccccc5c5ccccc5c4cc23)cc1]

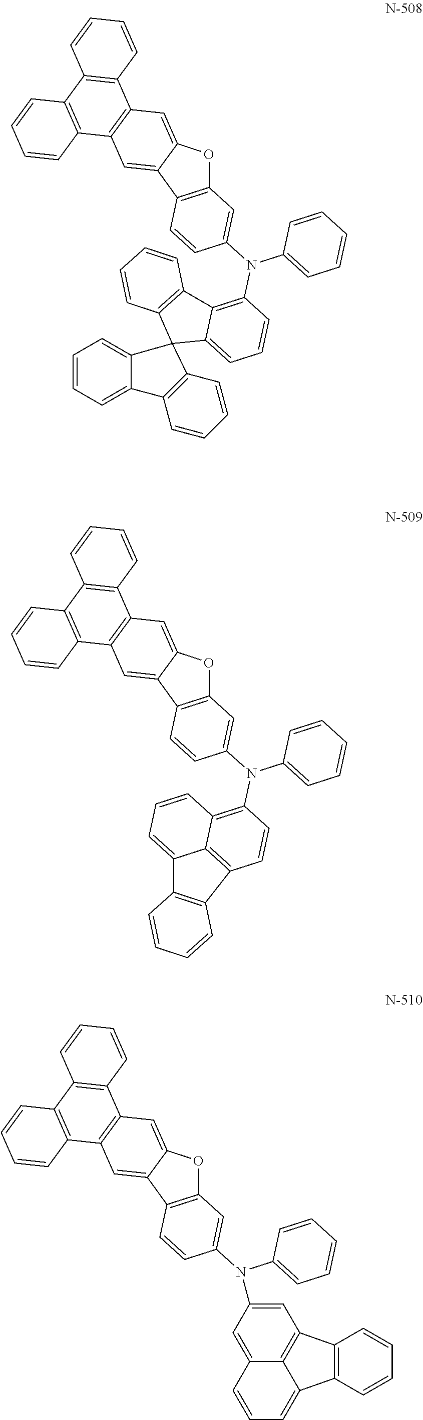 [SMILES: c1ccc(N(c2ccc3c(c2)oc2cc4c5ccccc5c5ccccc5c4cc23)c2cccc3c2-c2ccccc2C32c3ccccc3-c3ccccc32)cc1 ; c1ccc(N(c2ccc3c(c2)oc2cc4c5ccccc5c5ccccc5c4cc23)c2ccc3c4c(cccc24)-c2ccccc2-3)cc1 ; c1ccc(N(c2cc3c4c(cccc4c2)-c2ccccc2-3)c2ccc3c(c2)oc2cc4c5ccccc5c5ccccc5c4cc23)cc1]

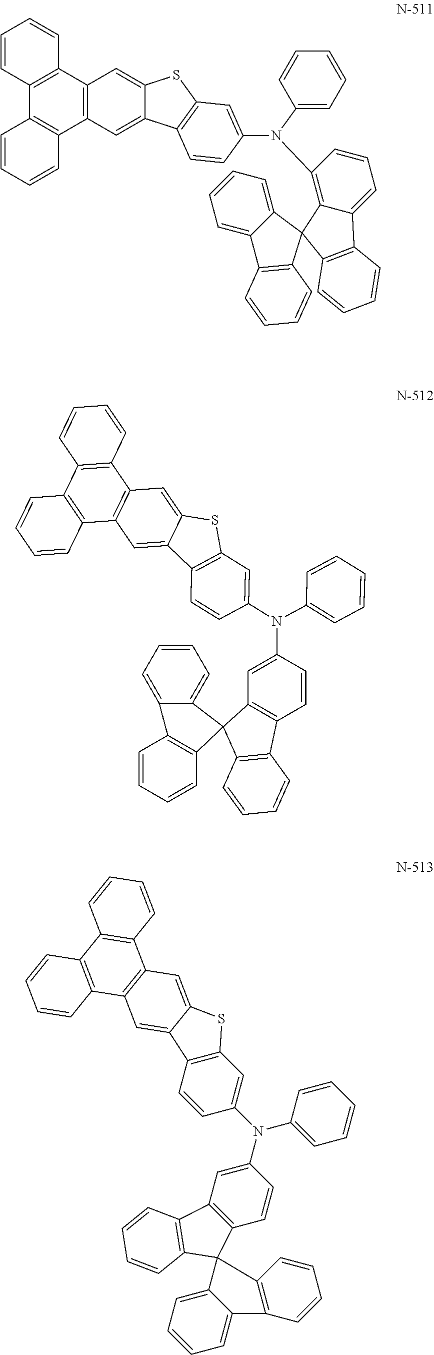 [SMILES: c1ccc(N(c2ccc3c(c2)sc2cc4c5ccccc5c5ccccc5c4cc23)c2cccc3c2C2(c4ccccc4-c4ccccc42)c2ccccc2-3)cc1 ; c1ccc(N(c2ccc3c(c2)C2(c4ccccc4-c4ccccc42)c2ccccc2-3)c2ccc3c(c2)sc2cc4c5ccccc5c5ccccc5c4cc23)cc1 ; c1ccc(N(c2ccc3c(c2)-c2ccccc2C32c3ccccc3-c3ccccc32)c2ccc3c(c2)sc2cc4c5ccccc5c5ccccc5c4cc23)cc1]

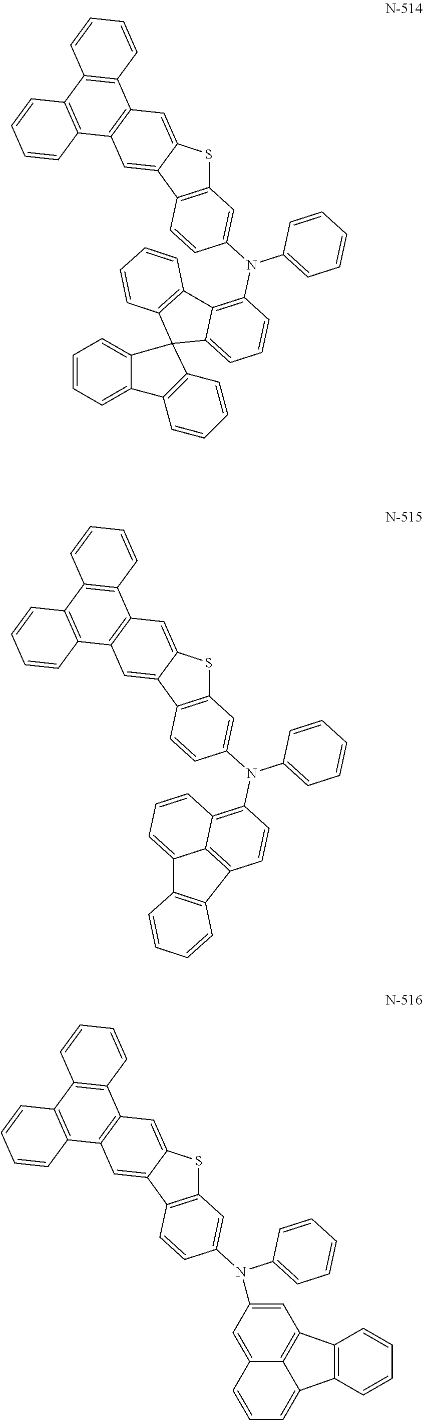 [SMILES: c1ccc(N(c2ccc3c(c2)sc2cc4c5ccccc5c5ccccc5c4cc23)c2cccc3c2-c2ccccc2C32c3ccccc3-c3ccccc32)cc1 ; c1ccc(N(c2ccc3c(c2)sc2cc4c5ccccc5c5ccccc5c4cc23)c2ccc3c4c(cccc24)-c2ccccc2-3)cc1 ; c1ccc(N(c2cc3c4c(cccc4c2)-c2ccccc2-3)c2ccc3c(c2)sc2cc4c5ccccc5c5ccccc5c4cc23)cc1]

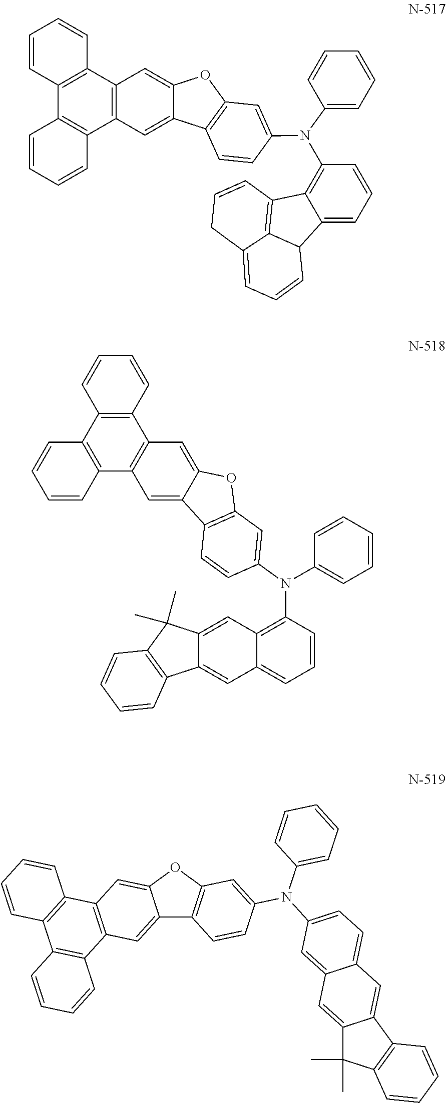 [SMILES: C1=CC2C3=C(C=CCC3=C1)c1c2cccc1N(c1ccccc1)c1ccc2c(c1)oc1cc3c4ccccc4c4ccccc4c3cc12 ; CC1(C)c2ccccc2-c2cc3cccc(N(c4ccccc4)c4ccc5c(c4)oc4cc6c7ccccc7c7ccccc7c6cc45)c3cc21 ; CC1(C)c2ccccc2-c2cc3ccc(N(c4ccccc4)c4ccc5c(c4)oc4cc6c7ccccc7c7ccccc7c6cc45)cc3cc21]

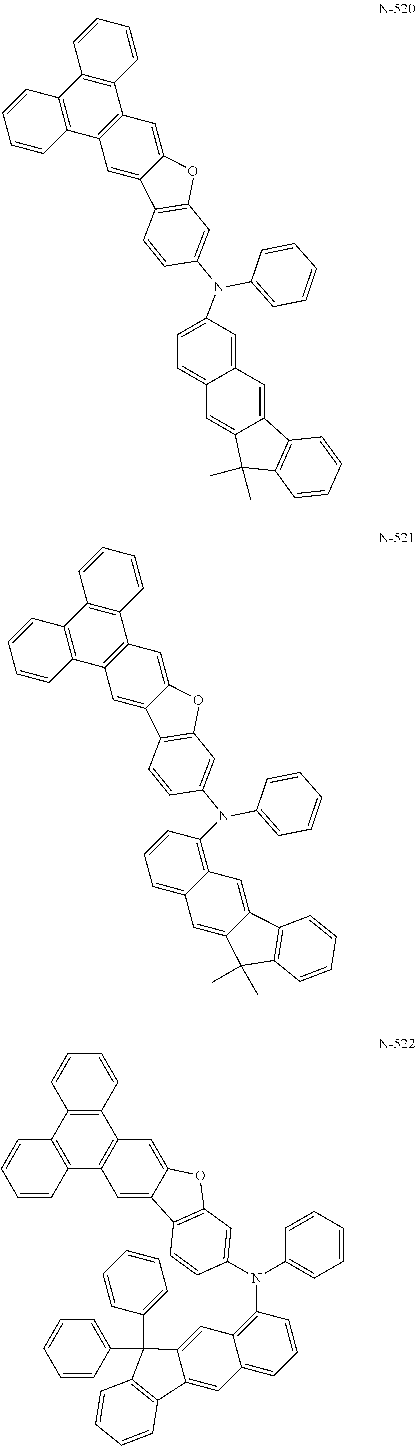 [SMILES: CC1(C)c2ccccc2-c2cc3cc(N(c4ccccc4)c4ccc5c(c4)oc4cc6c7ccccc7c7ccccc7c6cc45)ccc3cc21 ; CC1(C)c2ccccc2-c2cc3c(N(c4ccccc4)c4ccc5c(c4)oc4cc6c7ccccc7c7ccccc7c6cc45)cccc3cc21 ; c1ccc(N(c2ccc3c(c2)oc2cc4c5ccccc5c5ccccc5c4cc23)c2cccc3cc4c(cc23)C(c2ccccc2)(c2ccccc2)c2ccccc2-4)cc1]

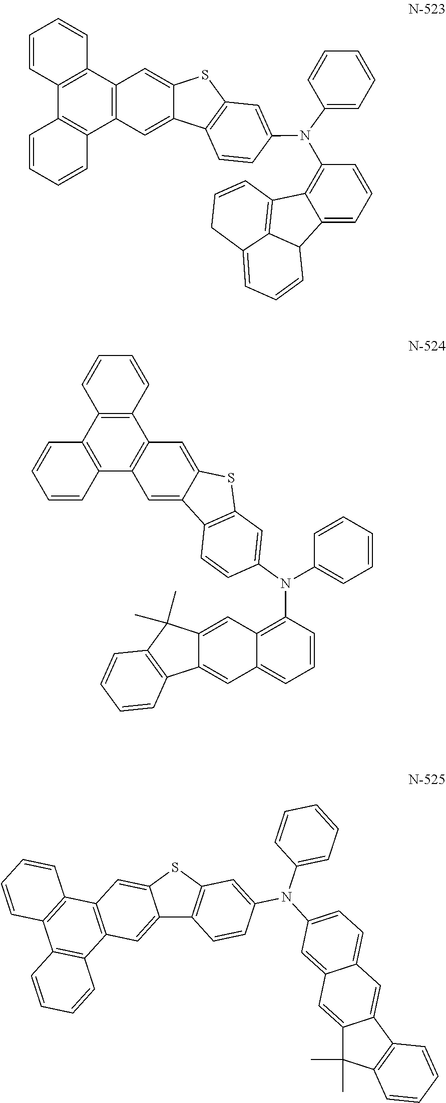 [SMILES: C1=CC2C3=C(C=CCC3=C1)c1c2cccc1N(c1ccccc1)c1ccc2c(c1)sc1cc3c4ccccc4c4ccccc4c3cc12 ; CC1(C)c2ccccc2-c2cc3cccc(N(c4ccccc4)c4ccc5c(c4)sc4cc6c7ccccc7c7ccccc7c6cc45)c3cc21 ; CC1(C)c2ccccc2-c2cc3ccc(N(c4ccccc4)c4ccc5c(c4)sc4cc6c7ccccc7c7ccccc7c6cc45)cc3cc21]

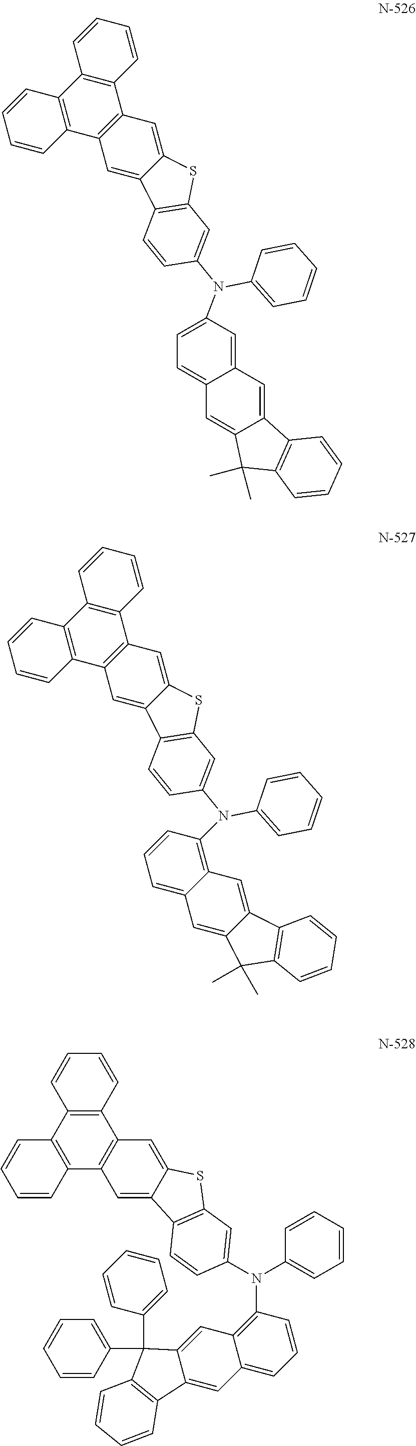 [SMILES: CC1(C)c2ccccc2-c2cc3cc(N(c4ccccc4)c4ccc5c(c4)sc4cc6c7ccccc7c7ccccc7c6cc45)ccc3cc21 ; CC1(C)c2ccccc2-c2cc3c(N(c4ccccc4)c4ccc5c(c4)sc4cc6c7ccccc7c7ccccc7c6cc45)cccc3cc21 ; c1ccc(N(c2ccc3c(c2)sc2cc4c5ccccc5c5ccccc5c4cc23)c2cccc3cc4c(cc23)C(c2ccccc2)(c2ccccc2)c2ccccc2-4)cc1]

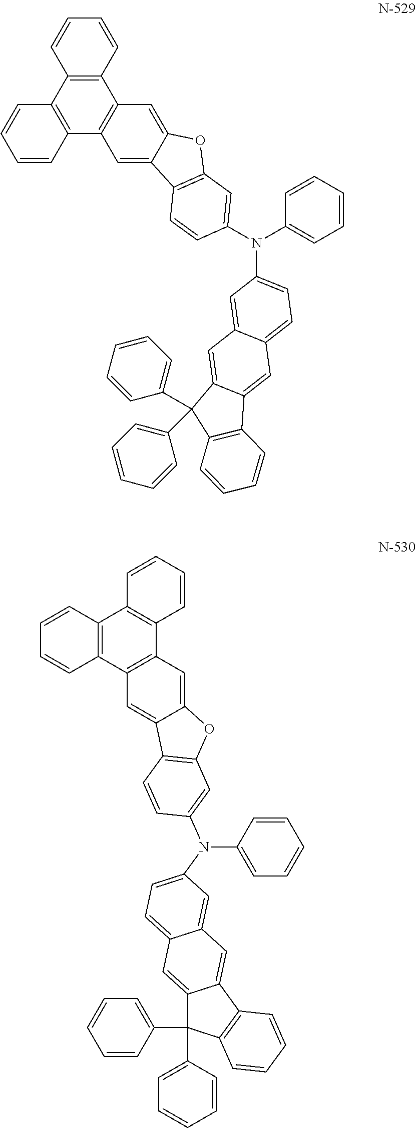 [SMILES: c1ccc(N(c2ccc3cc4c(cc3c2)C(c2ccccc2)(c2ccccc2)c2ccccc2-4)c2ccc3c(c2)oc2cc4c5ccccc5c5ccccc5c4cc23)cc1 ; c1ccc(N(c2ccc3cc4c(cc3c2)-c2ccccc2C4(c2ccccc2)c2ccccc2)c2ccc3c(c2)oc2cc4c5ccccc5c5ccccc5c4cc23)cc1]

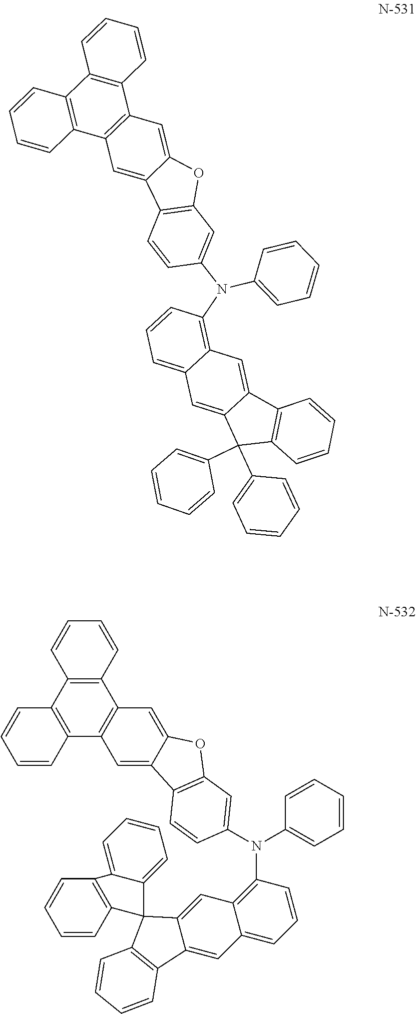 [SMILES: c1ccc(N(c2ccc3c(c2)oc2cc4c5ccccc5c5ccccc5c4cc23)c2cccc3cc4c(cc23)-c2ccccc2C4(c2ccccc2)c2ccccc2)cc1 ; c1ccc(N(c2ccc3c(c2)oc2cc4c5ccccc5c5ccccc5c4cc23)c2cccc3cc4c(cc23)C2(c3ccccc3-c3ccccc32)c2ccccc2-4)cc1]

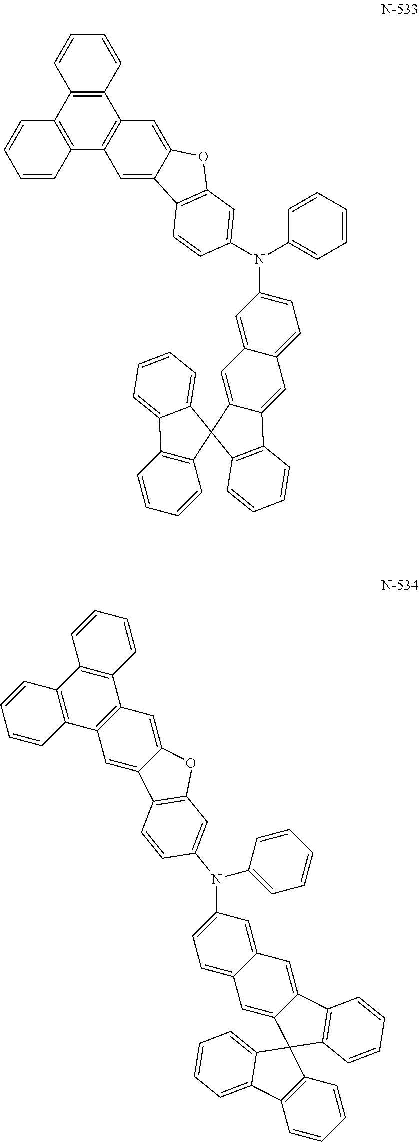 [SMILES: c1ccc(N(c2ccc3cc4c(cc3c2)C2(c3ccccc3-c3ccccc32)c2ccccc2-4)c2ccc3c(c2)oc2cc4c5ccccc5c5ccccc5c4cc23)cc1 ; c1ccc(N(c2ccc3cc4c(cc3c2)-c2ccccc2C42c3ccccc3-c3ccccc32)c2ccc3c(c2)oc2cc4c5ccccc5c5ccccc5c4cc23)cc1]

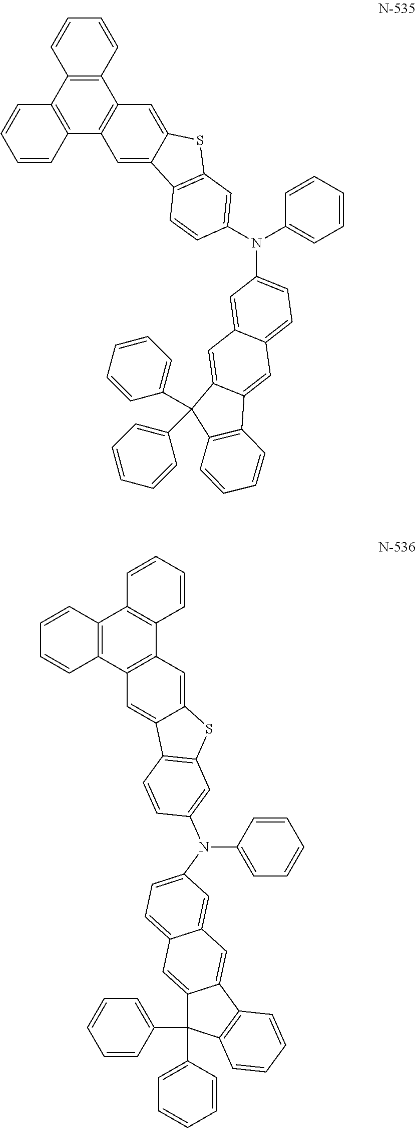 [SMILES: c1ccc(N(c2ccc3cc4c(cc3c2)C(c2ccccc2)(c2ccccc2)c2ccccc2-4)c2ccc3c(c2)sc2cc4c5ccccc5c5ccccc5c4cc23)cc1 ; c1ccc(N(c2ccc3cc4c(cc3c2)-c2ccccc2C4(c2ccccc2)c2ccccc2)c2ccc3c(c2)sc2cc4c5ccccc5c5ccccc5c4cc23)cc1]

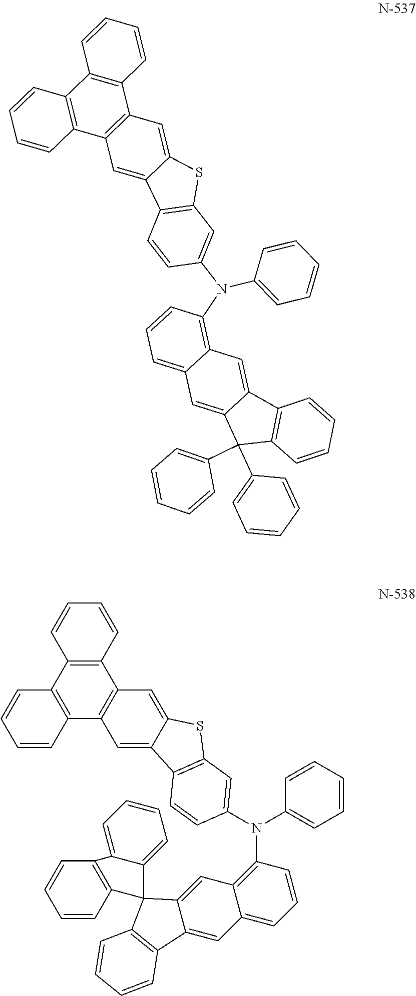 [SMILES: c1ccc(N(c2ccc3c(c2)sc2cc4c5ccccc5c5ccccc5c4cc23)c2cccc3cc4c(cc23)-c2ccccc2C4(c2ccccc2)c2ccccc2)cc1 ; c1ccc(N(c2ccc3c(c2)sc2cc4c5ccccc5c5ccccc5c4cc23)c2cccc3cc4c(cc23)C2(c3ccccc3-c3ccccc32)c2ccccc2-4)cc1]

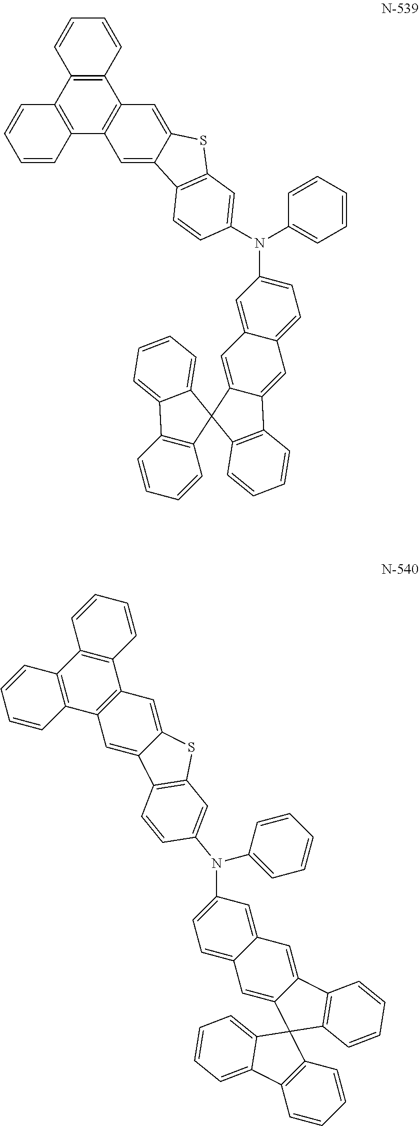 [SMILES: c1ccc(N(c2ccc3cc4c(cc3c2)C2(c3ccccc3-c3ccccc32)c2ccccc2-4)c2ccc3c(c2)sc2cc4c5ccccc5c5ccccc5c4cc23)cc1 ; c1ccc(N(c2ccc3cc4c(cc3c2)-c2ccccc2C42c3ccccc3-c3ccccc32)c2ccc3c(c2)sc2cc4c5ccccc5c5ccccc5c4cc23)cc1]

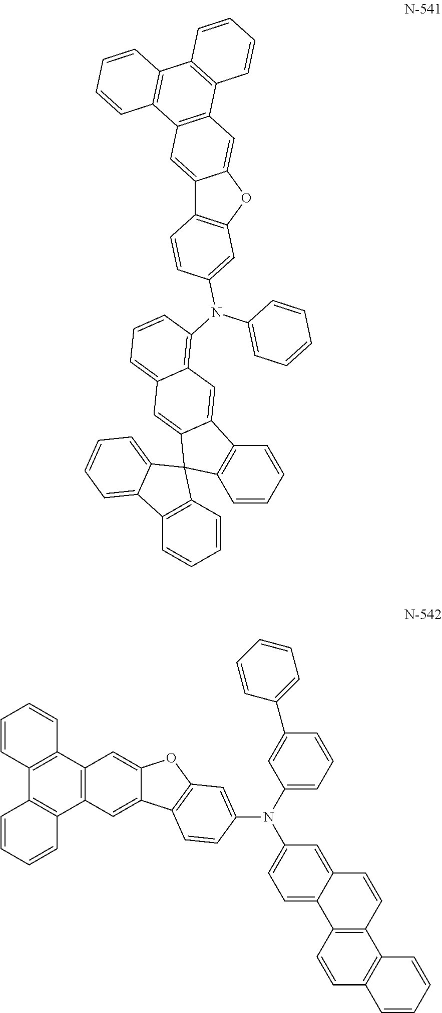 [SMILES: c1ccc(N(c2ccc3c(c2)oc2cc4c5ccccc5c5ccccc5c4cc23)c2cccc3cc4c(cc23)-c2ccccc2C42c3ccccc3-c3ccccc32)cc1 ; c1ccc(-c2cccc(N(c3ccc4c(ccc5c6ccccc6ccc45)c3)c3ccc4c(c3)oc3cc5c6ccccc6c6ccccc6c5cc34)c2)cc1]

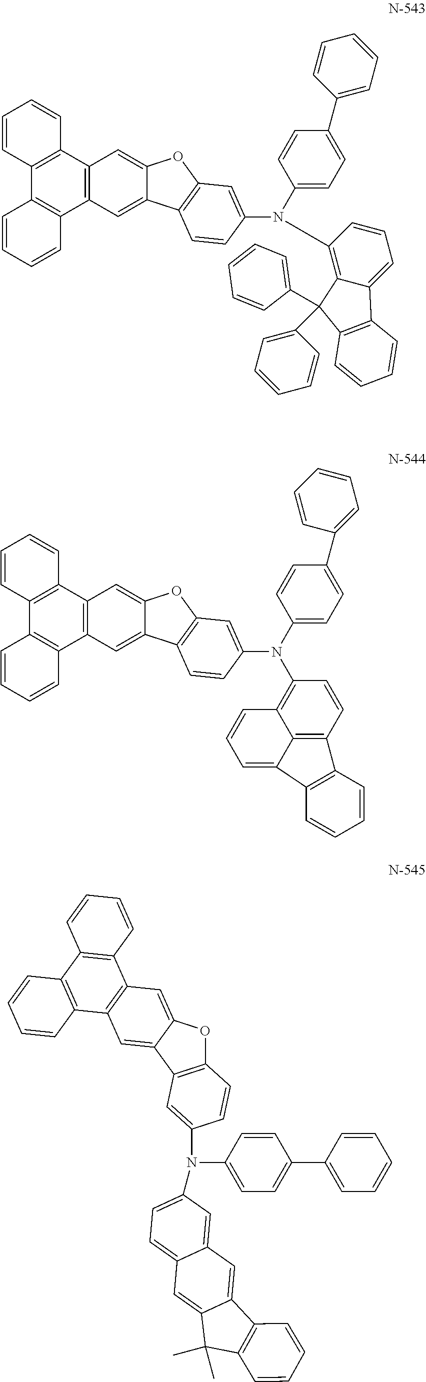 [SMILES: c1ccc(-c2ccc(N(c3ccc4c(c3)oc3cc5c6ccccc6c6ccccc6c5cc34)c3cccc4c3C(c3ccccc3)(c3ccccc3)c3ccccc3-4)cc2)cc1 ; c1ccc(-c2ccc(N(c3ccc4c(c3)oc3cc5c6ccccc6c6ccccc6c5cc34)c3ccc4c5c(cccc35)-c3ccccc3-4)cc2)cc1 ; CC1(C)c2ccccc2-c2cc3cc(N(c4ccc(-c5ccccc5)cc4)c4ccc5oc6cc7c8ccccc8c8ccccc8c7cc6c5c4)ccc3cc21]

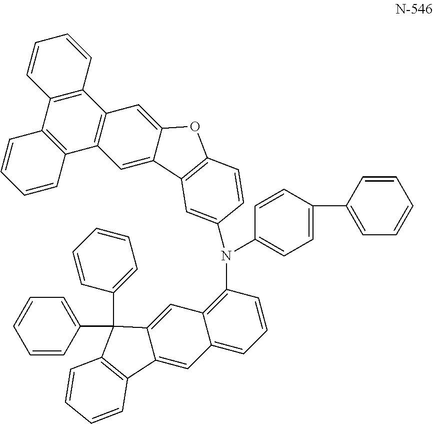 [SMILES: c1ccc(-c2ccc(N(c3ccc4oc5cc6c7ccccc7c7ccccc7c6cc5c4c3)c3cccc4cc5c(cc34)C(c3ccccc3)(c3ccccc3)c3ccccc3-5)cc2)cc1]

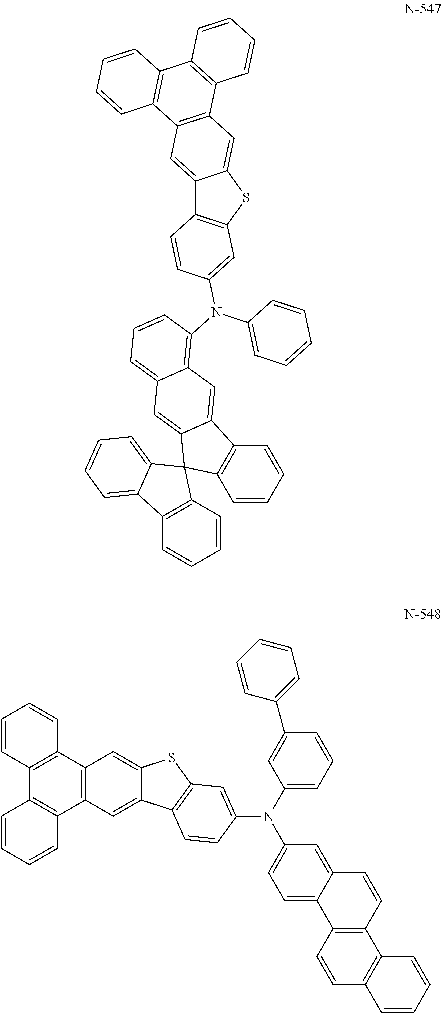 [SMILES: c1ccc(N(c2ccc3c(c2)sc2cc4c5ccccc5c5ccccc5c4cc23)c2cccc3cc4c(cc23)-c2ccccc2C42c3ccccc3-c3ccccc32)cc1 ; c1ccc(-c2cccc(N(c3ccc4c(ccc5c6ccccc6ccc45)c3)c3ccc4c(c3)sc3cc5c6ccccc6c6ccccc6c5cc34)c2)cc1]

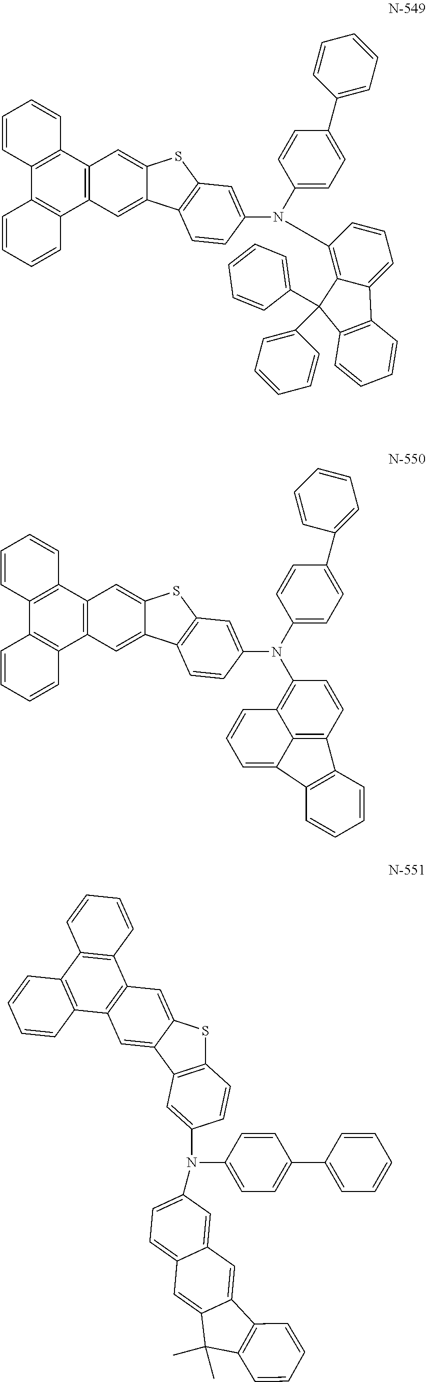 [SMILES: c1ccc(-c2ccc(N(c3ccc4c(c3)sc3cc5c6ccccc6c6ccccc6c5cc34)c3cccc4c3C(c3ccccc3)(c3ccccc3)c3ccccc3-4)cc2)cc1 ; c1ccc(-c2ccc(N(c3ccc4c(c3)sc3cc5c6ccccc6c6ccccc6c5cc34)c3ccc4c5c(cccc35)-c3ccccc3-4)cc2)cc1 ; CC1(C)c2ccccc2-c2cc3cc(N(c4ccc(-c5ccccc5)cc4)c4ccc5sc6cc7c8ccccc8c8ccccc8c7cc6c5c4)ccc3cc21]

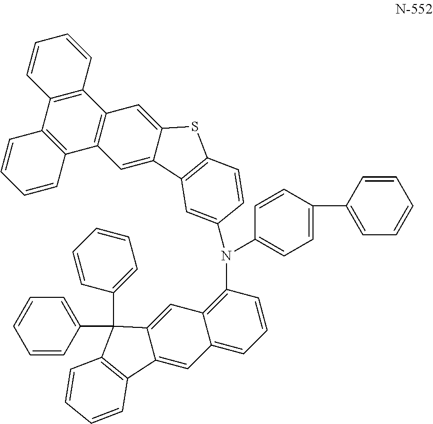 [SMILES: c1ccc(-c2ccc(N(c3ccc4sc5cc6c7ccccc7c7ccccc7c6cc5c4c3)c3cccc4cc5c(cc34)C(c3ccccc3)(c3ccccc3)c3ccccc3-5)cc2)cc1]

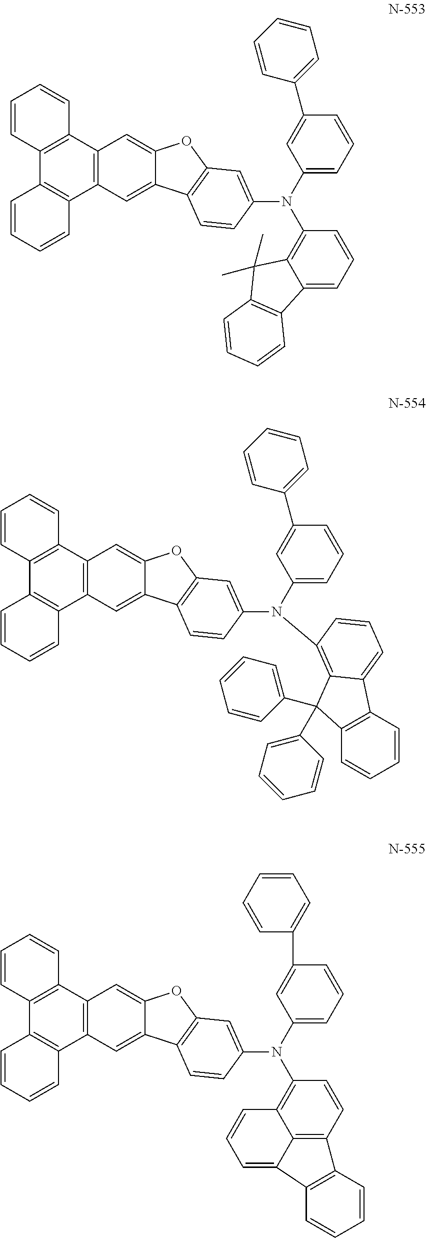 [SMILES: CC1(C)c2ccccc2-c2cccc(N(c3cccc(-c4ccccc4)c3)c3ccc4c(c3)oc3cc5c6ccccc6c6ccccc6c5cc34)c21 ; c1ccc(-c2cccc(N(c3ccc4c(c3)oc3cc5c6ccccc6c6ccccc6c5cc34)c3cccc4c3C(c3ccccc3)(c3ccccc3)c3ccccc3-4)c2)cc1 ; c1ccc(-c2cccc(N(c3ccc4c(c3)oc3cc5c6ccccc6c6ccccc6c5cc34)c3ccc4c5c(cccc35)-c3ccccc3-4)c2)cc1]

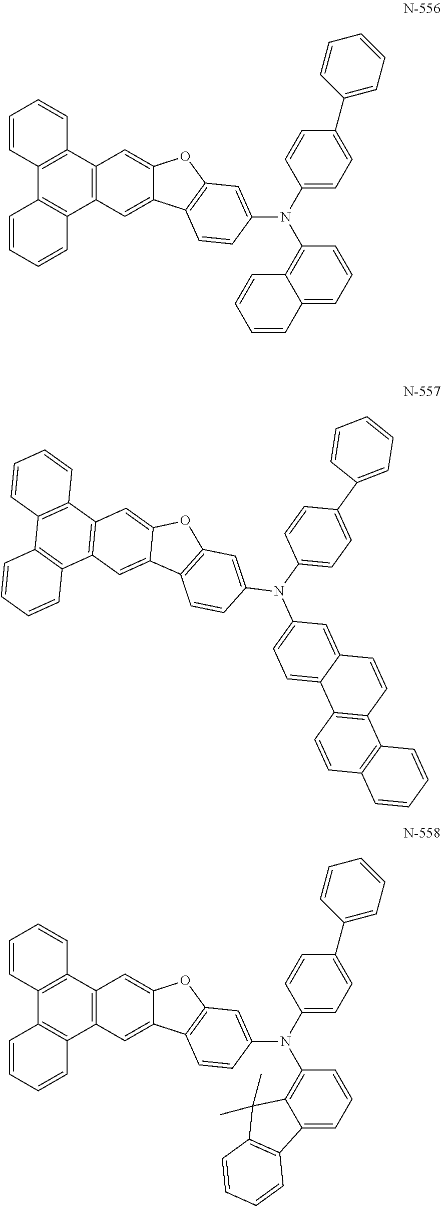 [SMILES: c1ccc(-c2ccc(N(c3ccc4c(c3)oc3cc5c6ccccc6c6ccccc6c5cc34)c3cccc4ccccc34)cc2)cc1 ; c1ccc(-c2ccc(N(c3ccc4c(ccc5c6ccccc6ccc45)c3)c3ccc4c(c3)oc3cc5c6ccccc6c6ccccc6c5cc34)cc2)cc1 ; CC1(C)c2ccccc2-c2cccc(N(c3ccc(-c4ccccc4)cc3)c3ccc4c(c3)oc3cc5c6ccccc6c6ccccc6c5cc34)c21]

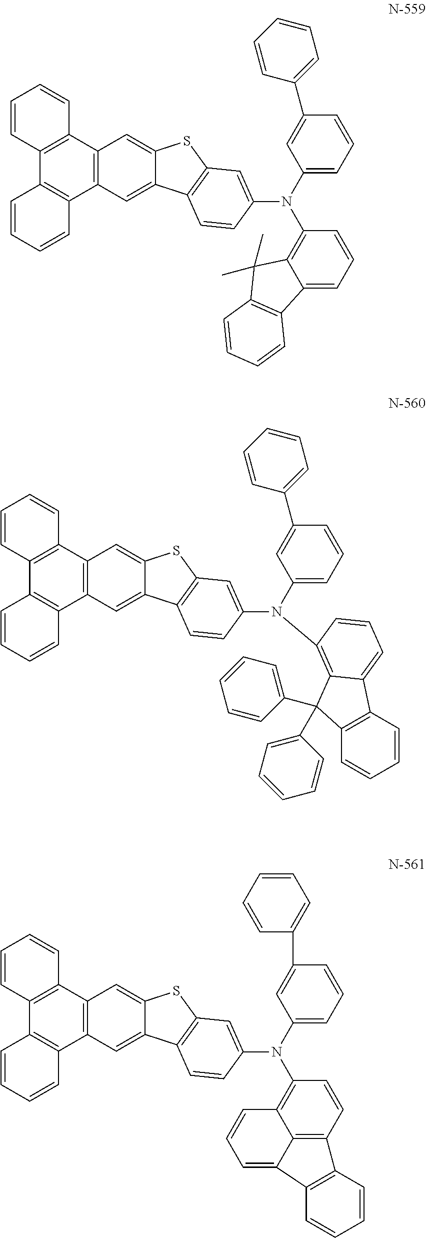 [SMILES: CC1(C)c2ccccc2-c2cccc(N(c3cccc(-c4ccccc4)c3)c3ccc4c(c3)sc3cc5c6ccccc6c6ccccc6c5cc34)c21 ; c1ccc(-c2cccc(N(c3ccc4c(c3)sc3cc5c6ccccc6c6ccccc6c5cc34)c3cccc4c3C(c3ccccc3)(c3ccccc3)c3ccccc3-4)c2)cc1 ; c1ccc(-c2cccc(N(c3ccc4c(c3)sc3cc5c6ccccc6c6ccccc6c5cc34)c3ccc4c5c(cccc35)-c3ccccc3-4)c2)cc1]

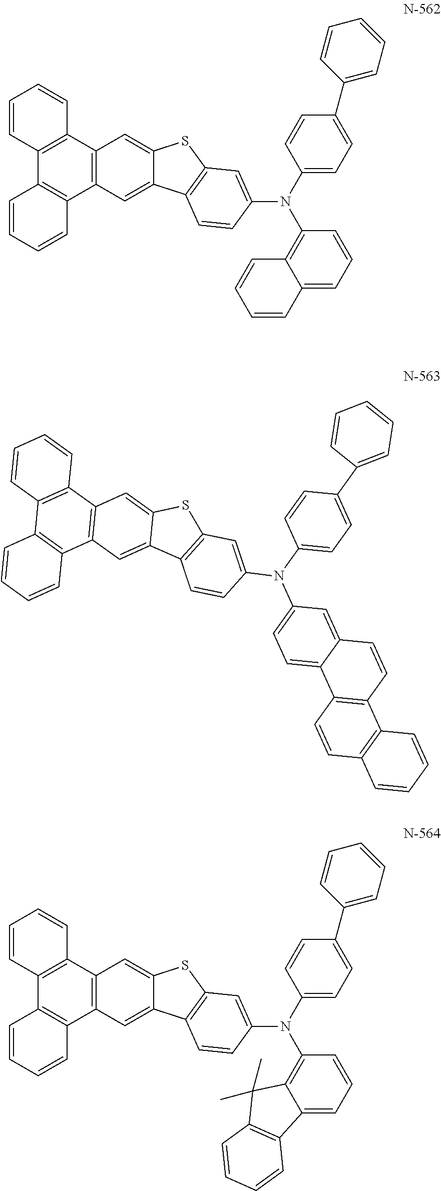 [SMILES: c1ccc(-c2ccc(N(c3ccc4c(c3)sc3cc5c6ccccc6c6ccccc6c5cc34)c3cccc4ccccc34)cc2)cc1 ; c1ccc(-c2ccc(N(c3ccc4c(ccc5c6ccccc6ccc45)c3)c3ccc4c(c3)sc3cc5c6ccccc6c6ccccc6c5cc34)cc2)cc1 ; CC1(C)c2ccccc2-c2cccc(N(c3ccc(-c4ccccc4)cc3)c3ccc4c(c3)sc3cc5c6ccccc6c6ccccc6c5cc34)c21]

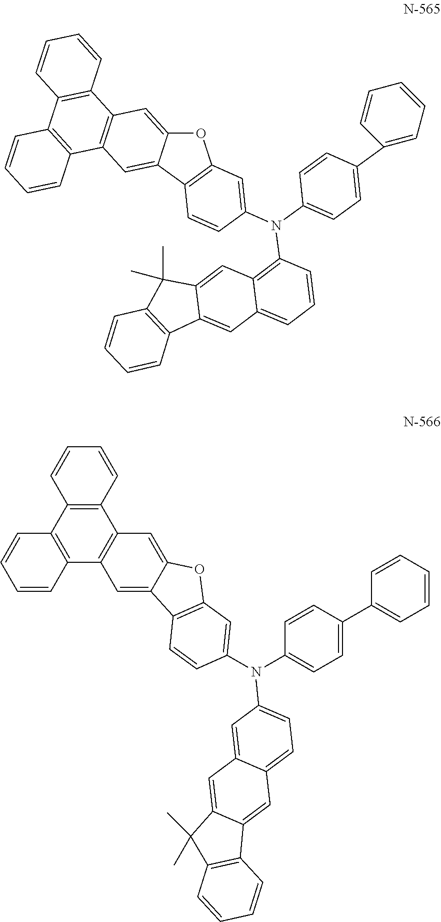 [SMILES: CC1(C)c2ccccc2-c2cc3cccc(N(c4ccc(-c5ccccc5)cc4)c4ccc5c(c4)oc4cc6c7ccccc7c7ccccc7c6cc45)c3cc21 ; CC1(C)c2ccccc2-c2cc3ccc(N(c4ccc(-c5ccccc5)cc4)c4ccc5c(c4)oc4cc6c7ccccc7c7ccccc7c6cc45)cc3cc21]

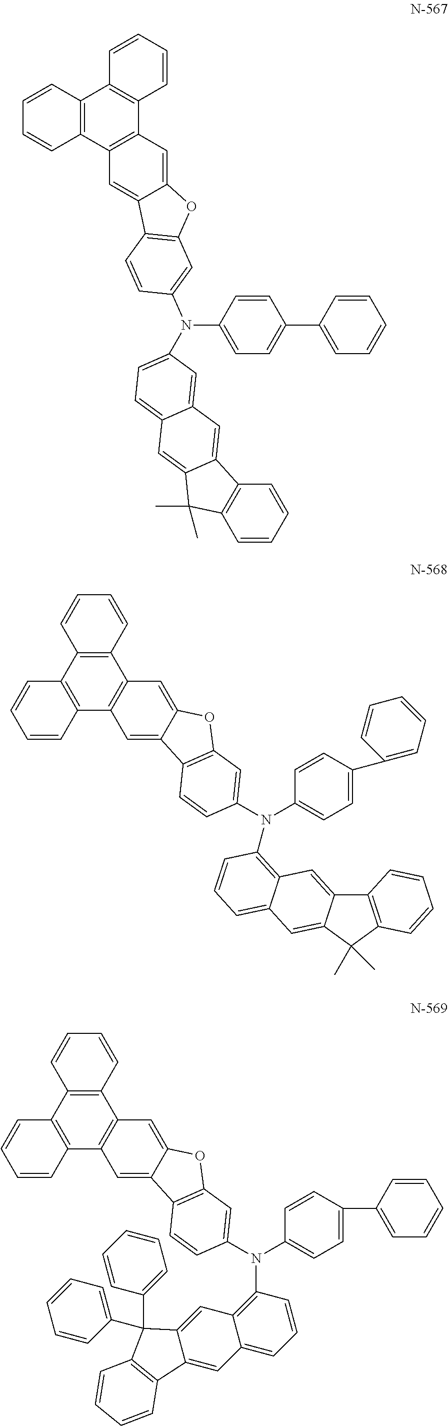 [SMILES: CC1(C)c2ccccc2-c2cc3cc(N(c4ccc(-c5ccccc5)cc4)c4ccc5c(c4)oc4cc6c7ccccc7c7ccccc7c6cc45)ccc3cc21 ; CC1(C)c2ccccc2-c2cc3c(N(c4ccc(-c5ccccc5)cc4)c4ccc5c(c4)oc4cc6c7ccccc7c7ccccc7c6cc45)cccc3cc21 ; c1ccc(-c2ccc(N(c3ccc4c(c3)oc3cc5c6ccccc6c6ccccc6c5cc34)c3cccc4cc5c(cc34)C(c3ccccc3)(c3ccccc3)c3ccccc3-5)cc2)cc1]

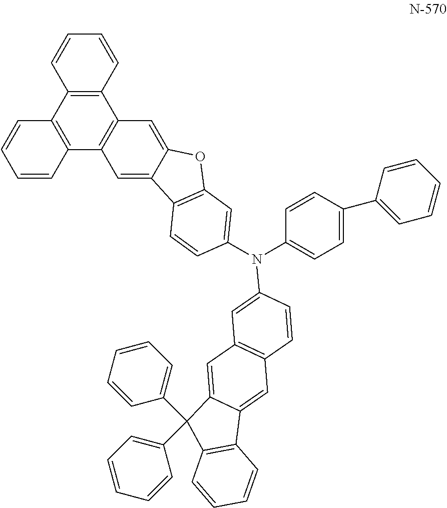 [SMILES: c1ccc(-c2ccc(N(c3ccc4cc5c(cc4c3)C(c3ccccc3)(c3ccccc3)c3ccccc3-5)c3ccc4c(c3)oc3cc5c6ccccc6c6ccccc6c5cc34)cc2)cc1]

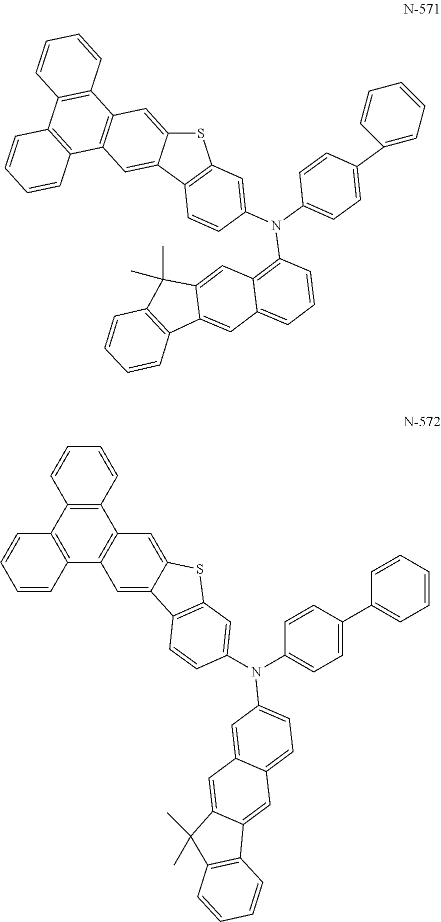 [SMILES: CC1(C)c2ccccc2-c2cc3cccc(N(c4ccc(-c5ccccc5)cc4)c4ccc5c(c4)sc4cc6c7ccccc7c7ccccc7c6cc45)c3cc21 ; CC1(C)c2ccccc2-c2cc3ccc(N(c4ccc(-c5ccccc5)cc4)c4ccc5c(c4)sc4cc6c7ccccc7c7ccccc7c6cc45)cc3cc21]

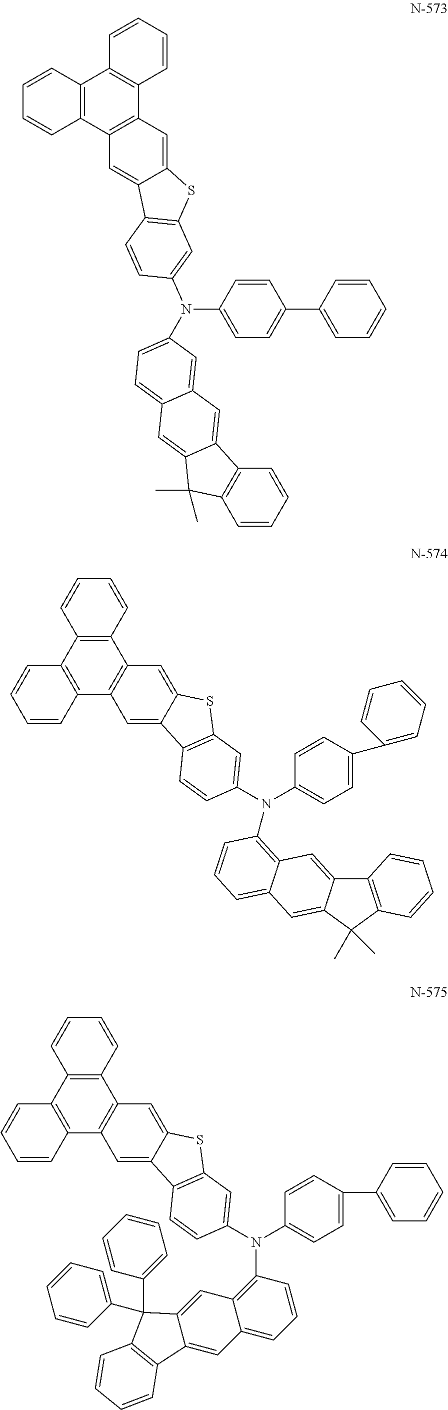 [SMILES: CC1(C)c2ccccc2-c2cc3cc(N(c4ccc(-c5ccccc5)cc4)c4ccc5c(c4)sc4cc6c7ccccc7c7ccccc7c6cc45)ccc3cc21 ; CC1(C)c2ccccc2-c2cc3c(N(c4ccc(-c5ccccc5)cc4)c4ccc5c(c4)sc4cc6c7ccccc7c7ccccc7c6cc45)cccc3cc21 ; c1ccc(-c2ccc(N(c3ccc4c(c3)sc3cc5c6ccccc6c6ccccc6c5cc34)c3cccc4cc5c(cc34)C(c3ccccc3)(c3ccccc3)c3ccccc3-5)cc2)cc1]

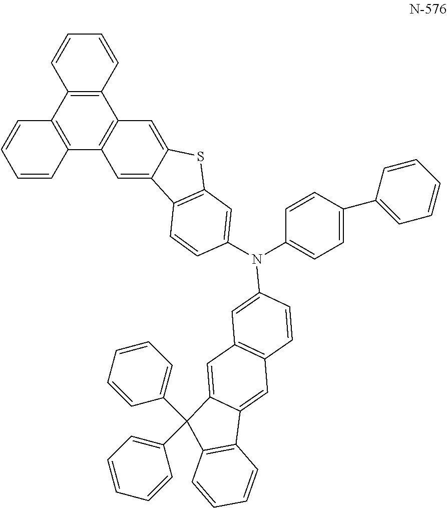 [SMILES: c1ccc(-c2ccc(N(c3ccc4cc5c(cc4c3)C(c3ccccc3)(c3ccccc3)c3ccccc3-5)c3ccc4c(c3)sc3cc5c6ccccc6c6ccccc6c5cc34)cc2)cc1]

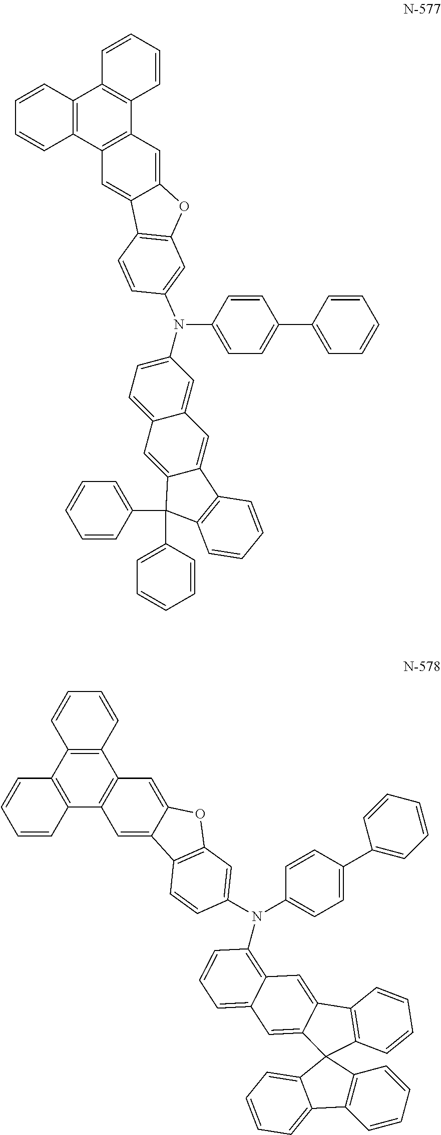 [SMILES: c1ccc(-c2ccc(N(c3ccc4cc5c(cc4c3)-c3ccccc3C5(c3ccccc3)c3ccccc3)c3ccc4c(c3)oc3cc5c6ccccc6c6ccccc6c5cc34)cc2)cc1 ; c1ccc(-c2ccc(N(c3ccc4c(c3)oc3cc5c6ccccc6c6ccccc6c5cc34)c3cccc4cc5c(cc34)-c3ccccc3C53c4ccccc4-c4ccccc43)cc2)cc1]

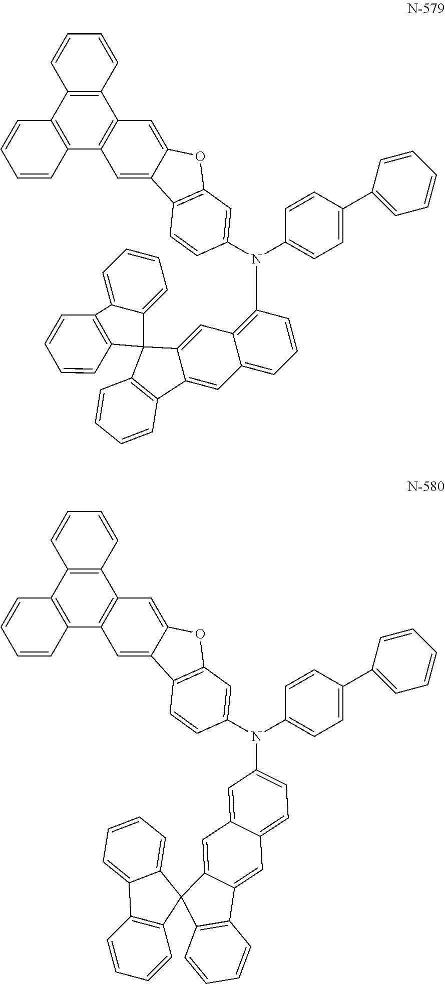 [SMILES: c1ccc(-c2ccc(N(c3ccc4c(c3)oc3cc5c6ccccc6c6ccccc6c5cc34)c3cccc4cc5c(cc34)C3(c4ccccc4-c4ccccc43)c3ccccc3-5)cc2)cc1 ; c1ccc(-c2ccc(N(c3ccc4cc5c(cc4c3)C3(c4ccccc4-c4ccccc43)c3ccccc3-5)c3ccc4c(c3)oc3cc5c6ccccc6c6ccccc6c5cc34)cc2)cc1]

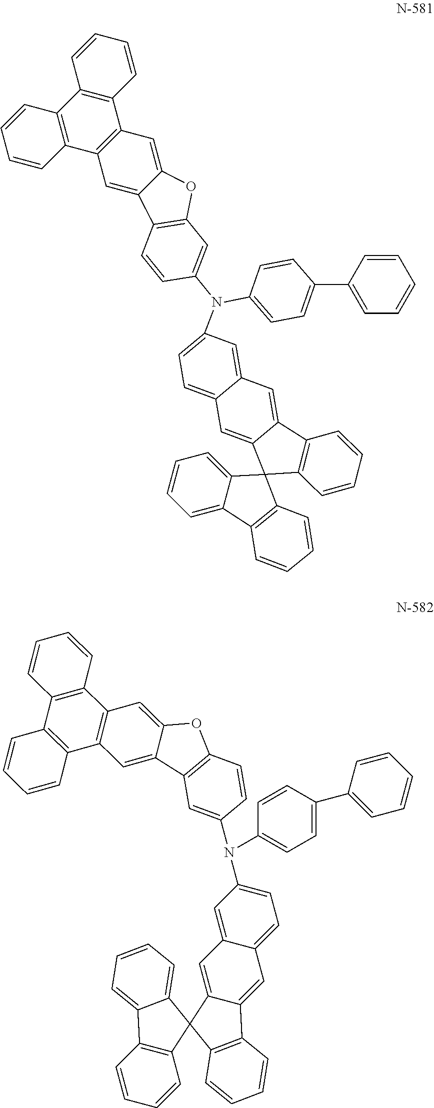 [SMILES: c1ccc(-c2ccc(N(c3ccc4cc5c(cc4c3)-c3ccccc3C53c4ccccc4-c4ccccc43)c3ccc4c(c3)oc3cc5c6ccccc6c6ccccc6c5cc34)cc2)cc1 ; c1ccc(-c2ccc(N(c3ccc4cc5c(cc4c3)C3(c4ccccc4-c4ccccc43)c3ccccc3-5)c3ccc4oc5cc6c7ccccc7c7ccccc7c6cc5c4c3)cc2)cc1]

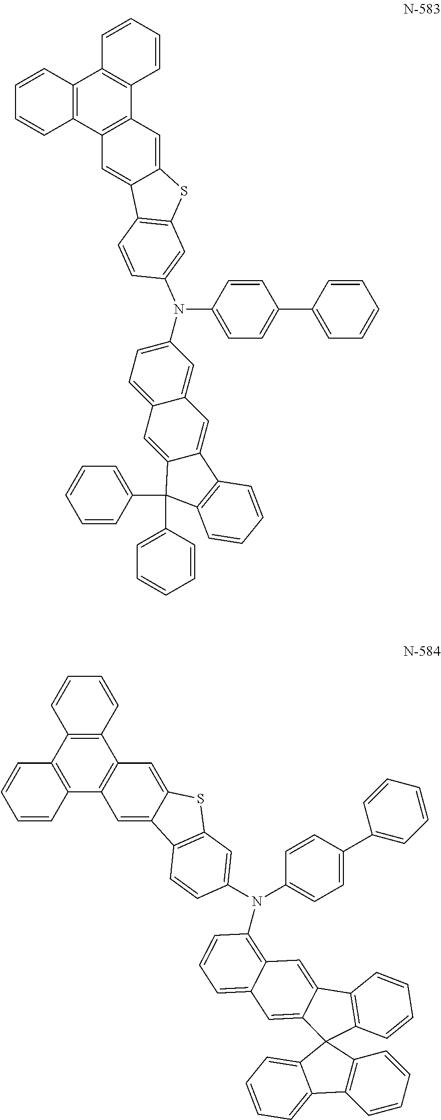 [SMILES: c1ccc(-c2ccc(N(c3ccc4cc5c(cc4c3)-c3ccccc3C5(c3ccccc3)c3ccccc3)c3ccc4c(c3)sc3cc5c6ccccc6c6ccccc6c5cc34)cc2)cc1 ; c1ccc(-c2ccc(N(c3ccc4c(c3)sc3cc5c6ccccc6c6ccccc6c5cc34)c3cccc4cc5c(cc34)-c3ccccc3C53c4ccccc4-c4ccccc43)cc2)cc1]

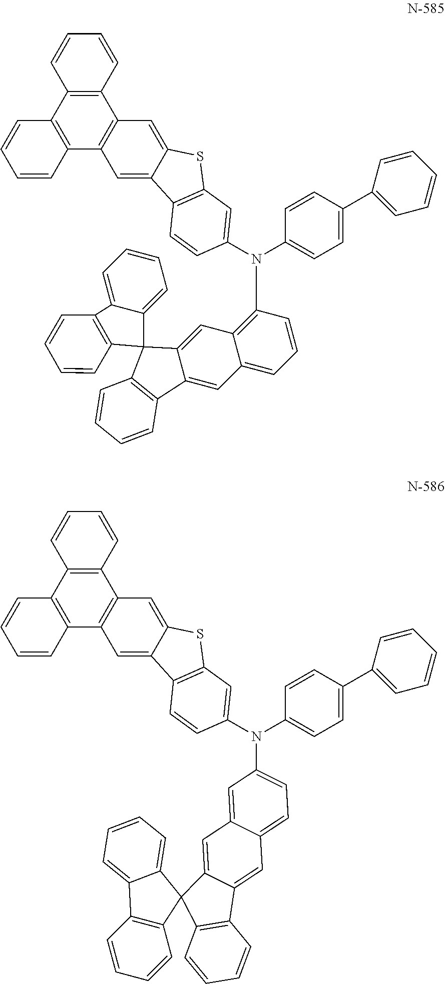 [SMILES: c1ccc(-c2ccc(N(c3ccc4c(c3)sc3cc5c6ccccc6c6ccccc6c5cc34)c3cccc4cc5c(cc34)C3(c4ccccc4-c4ccccc43)c3ccccc3-5)cc2)cc1 ; c1ccc(-c2ccc(N(c3ccc4cc5c(cc4c3)C3(c4ccccc4-c4ccccc43)c3ccccc3-5)c3ccc4c(c3)sc3cc5c6ccccc6c6ccccc6c5cc34)cc2)cc1]

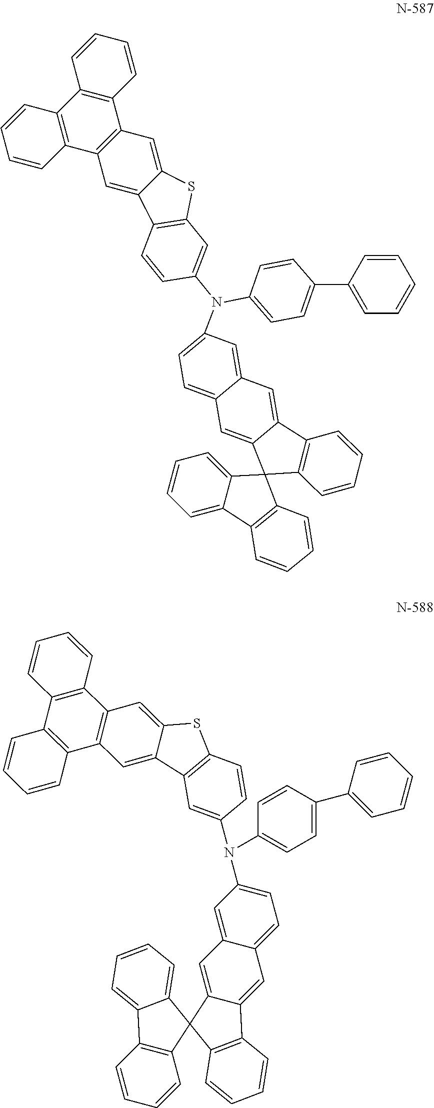 [SMILES: c1ccc(-c2ccc(N(c3ccc4cc5c(cc4c3)-c3ccccc3C53c4ccccc4-c4ccccc43)c3ccc4c(c3)sc3cc5c6ccccc6c6ccccc6c5cc34)cc2)cc1 ; c1ccc(-c2ccc(N(c3ccc4cc5c(cc4c3)C3(c4ccccc4-c4ccccc43)c3ccccc3-5)c3ccc4sc5cc6c7ccccc7c7ccccc7c6cc5c4c3)cc2)cc1]

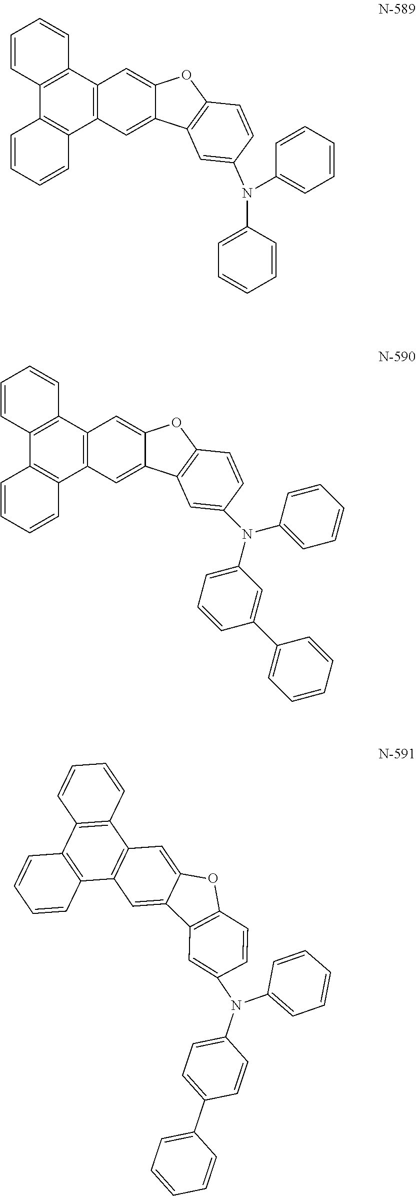 [SMILES: c1ccc(N(c2ccccc2)c2ccc3oc4cc5c6ccccc6c6ccccc6c5cc4c3c2)cc1 ; c1ccc(-c2cccc(N(c3ccccc3)c3ccc4oc5cc6c7ccccc7c7ccccc7c6cc5c4c3)c2)cc1 ; c1ccc(-c2ccc(N(c3ccccc3)c3ccc4oc5cc6c7ccccc7c7ccccc7c6cc5c4c3)cc2)cc1]

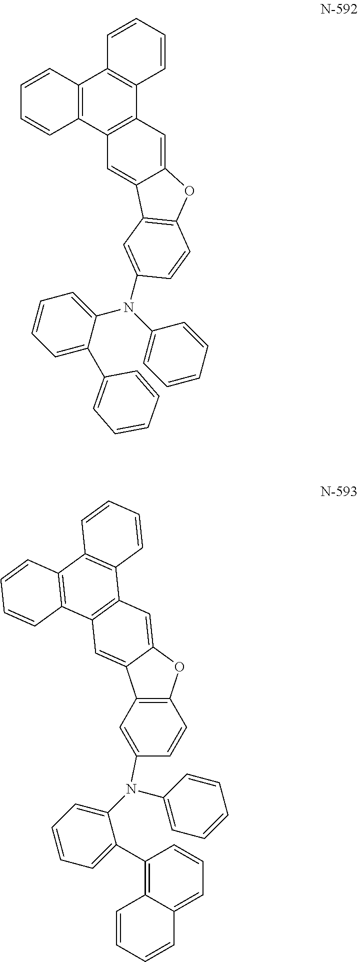 [SMILES: c1ccc(-c2ccccc2N(c2ccccc2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2ccccc2-c2cccc3ccccc23)cc1]

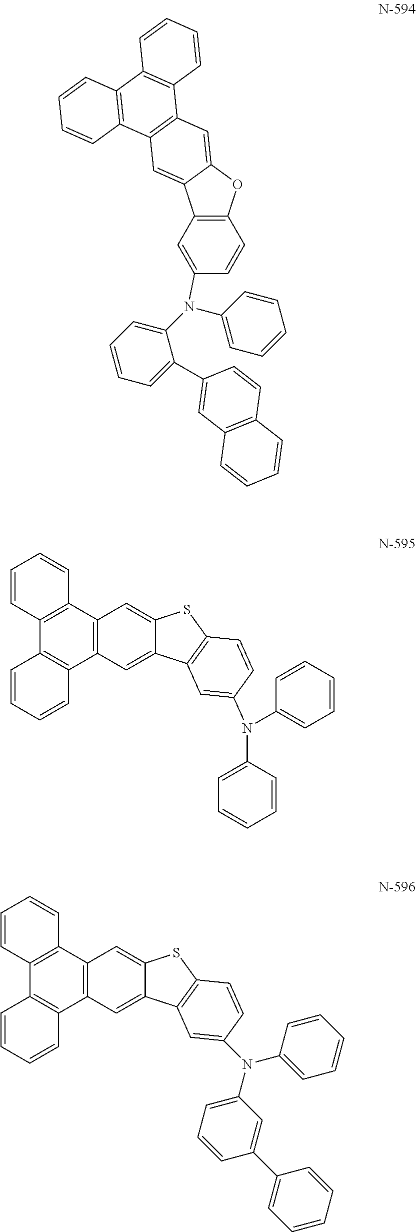 [SMILES: c1ccc(N(c2ccc3oc4cc5c6ccccc6c6ccccc6c5cc4c3c2)c2ccccc2-c2ccc3ccccc3c2)cc1 ; c1ccc(N(c2ccccc2)c2ccc3sc4cc5c6ccccc6c6ccccc6c5cc4c3c2)cc1 ; c1ccc(-c2cccc(N(c3ccccc3)c3ccc4sc5cc6c7ccccc7c7ccccc7c6cc5c4c3)c2)cc1]

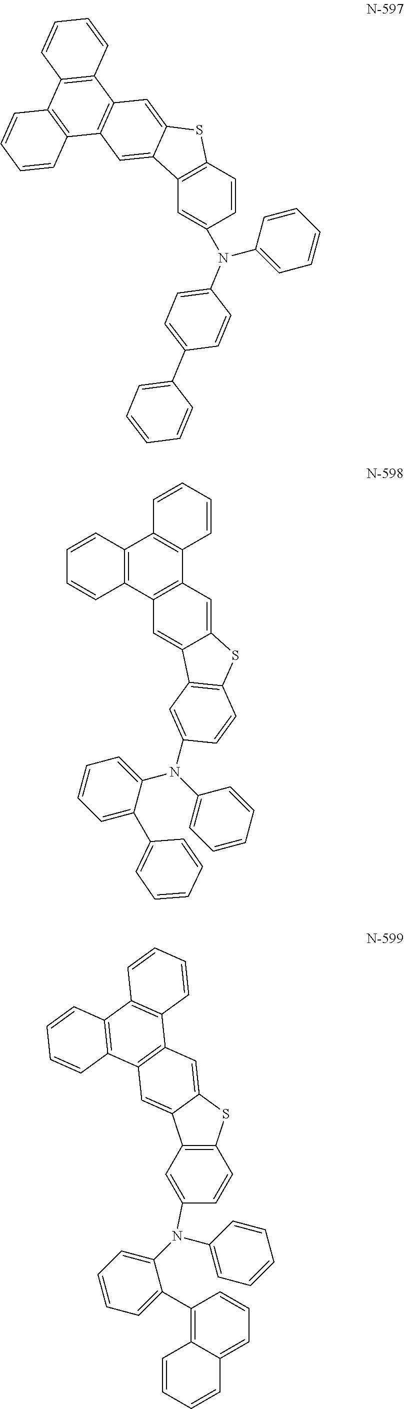

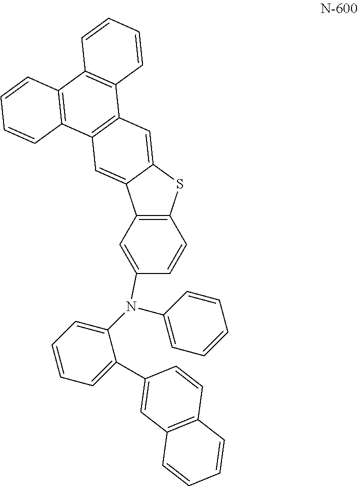 [SMILES: c1ccc(N(c2ccc3sc4cc5c6ccccc6c6ccccc6c5cc4c3c2)c2ccccc2-c2ccc3ccccc3c2)cc1]

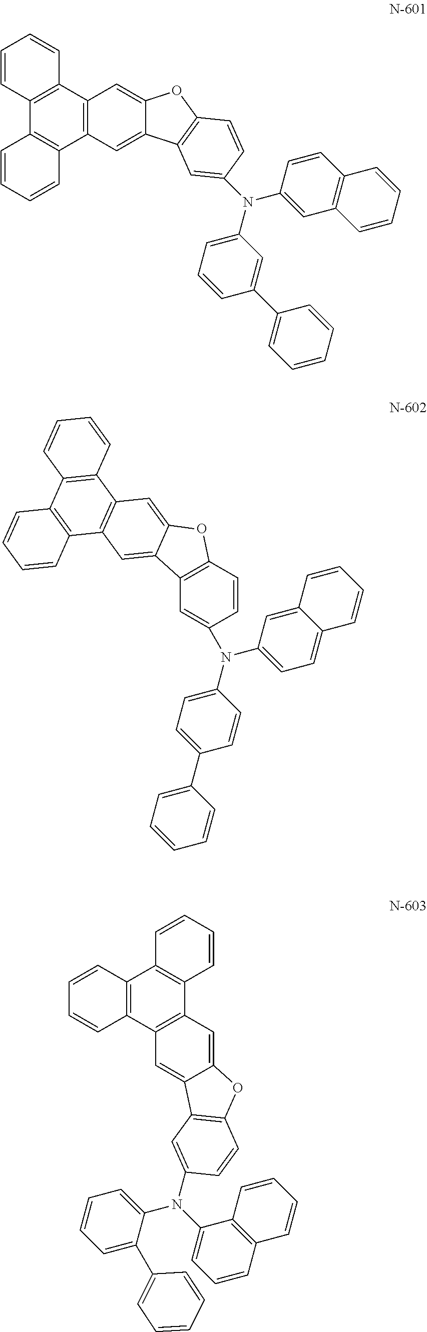 [SMILES: c1ccc(-c2cccc(N(c3ccc4ccccc4c3)c3ccc4oc5cc6c7ccccc7c7ccccc7c6cc5c4c3)c2)cc1 ; c1ccc(-c2ccc(N(c3ccc4ccccc4c3)c3ccc4oc5cc6c7ccccc7c7ccccc7c6cc5c4c3)cc2)cc1 ; c1ccc(-c2ccccc2N(c2ccc3oc4cc5c6ccccc6c6ccccc6c5cc4c3c2)c2cccc3ccccc23)cc1]

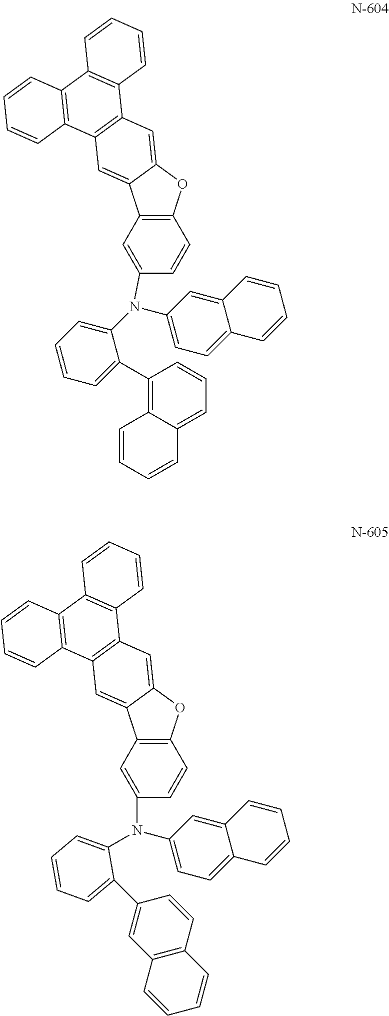 [SMILES: c1ccc(N(c2ccc3ccccc3c2)c2ccc3oc4cc5c6ccccc6c6ccccc6c5cc4c3c2)c(-c2cccc3ccccc23)c1 ; c1ccc(N(c2ccc3ccccc3c2)c2ccc3oc4cc5c6ccccc6c6ccccc6c5cc4c3c2)c(-c2ccc3ccccc3c2)c1]

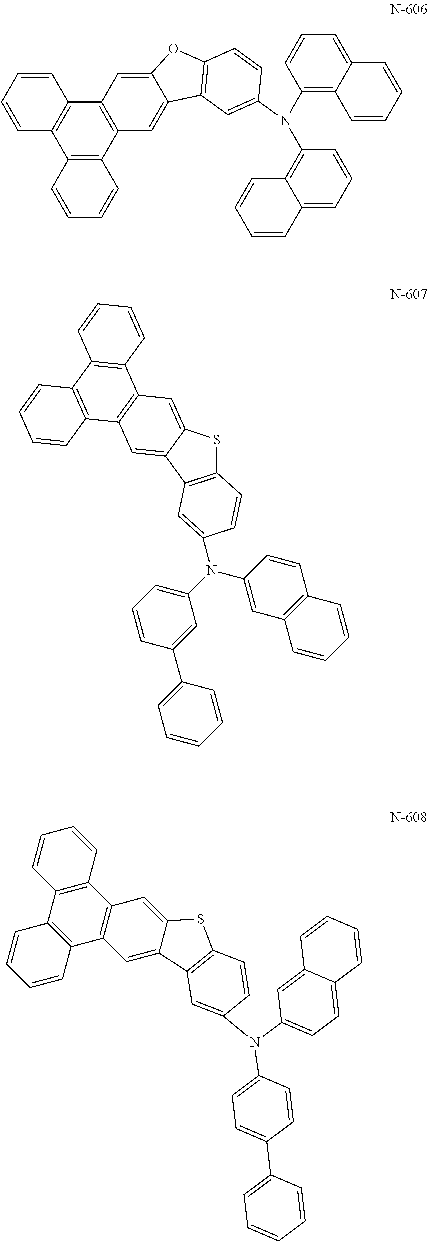 [SMILES: c1ccc2c(N(c3ccc4oc5cc6c7ccccc7c7ccccc7c6cc5c4c3)c3cccc4ccccc34)cccc2c1 ; c1ccc(-c2cccc(N(c3ccc4ccccc4c3)c3ccc4sc5cc6c7ccccc7c7ccccc7c6cc5c4c3)c2)cc1 ; c1ccc(-c2ccc(N(c3ccc4ccccc4c3)c3ccc4sc5cc6c7ccccc7c7ccccc7c6cc5c4c3)cc2)cc1]

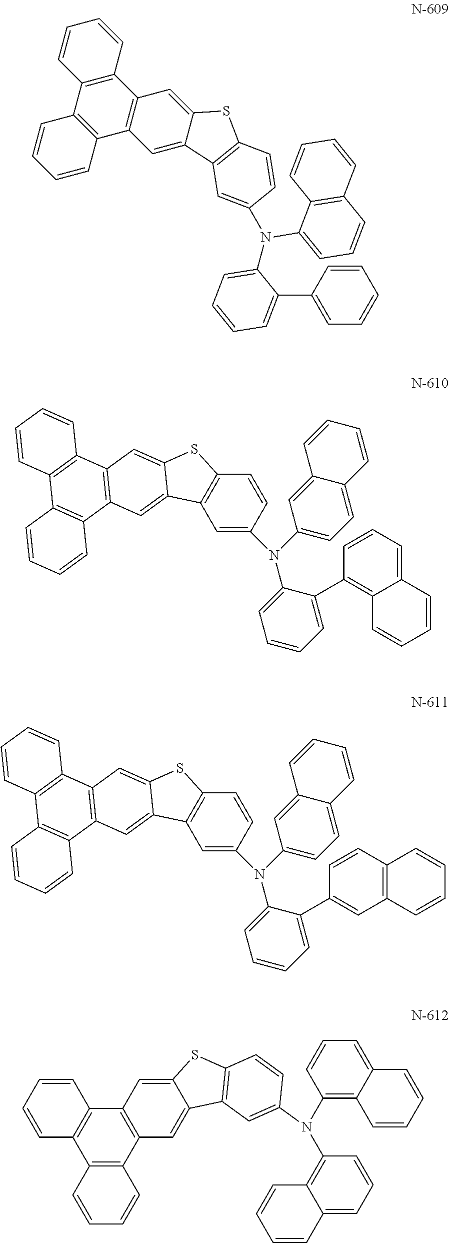 [SMILES: c1ccc(-c2ccccc2N(c2ccc3sc4cc5c6ccccc6c6ccccc6c5cc4c3c2)c2cccc3ccccc23)cc1 ; c1ccc(N(c2ccc3ccccc3c2)c2ccc3sc4cc5c6ccccc6c6ccccc6c5cc4c3c2)c(-c2cccc3ccccc23)c1 ; c1ccc(N(c2ccc3ccccc3c2)c2ccc3sc4cc5c6ccccc6c6ccccc6c5cc4c3c2)c(-c2ccc3ccccc3c2)c1 ; c1ccc2c(N(c3ccc4sc5cc6c7ccccc7c7ccccc7c6cc5c4c3)c3cccc4ccccc34)cccc2c1]

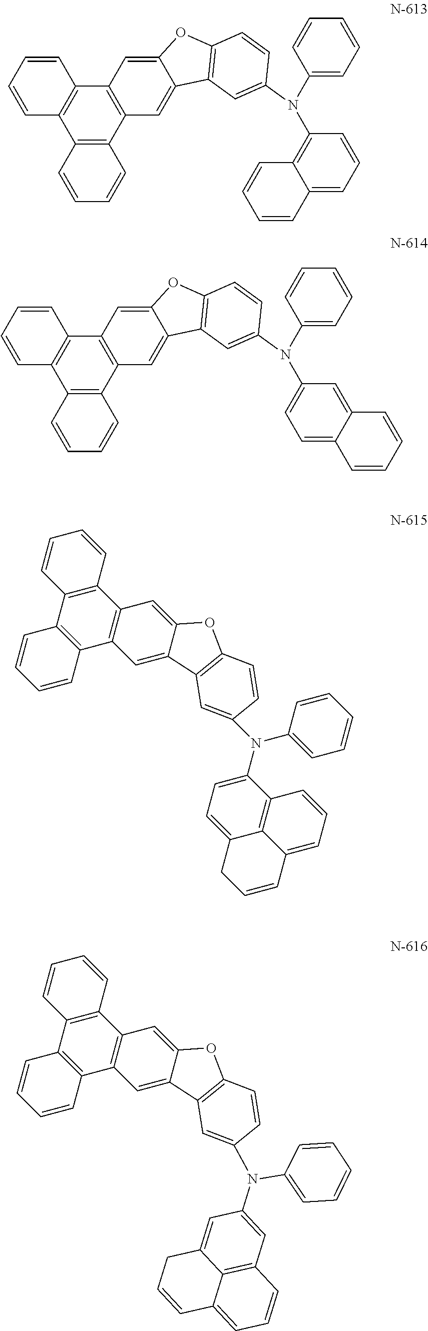 [SMILES: c1ccc(N(c2ccc3oc4cc5c6ccccc6c6ccccc6c5cc4c3c2)c2cccc3ccccc23)cc1 ; c1ccc(N(c2ccc3ccccc3c2)c2ccc3oc4cc5c6ccccc6c6ccccc6c5cc4c3c2)cc1 ; C1=Cc2cccc3c(N(c4ccccc4)c4ccc5oc6cc7c8ccccc8c8ccccc8c7cc6c5c4)ccc(c23)C1 ; C1=Cc2cccc3cc(N(c4ccccc4)c4ccc5oc6cc7c8ccccc8c8ccccc8c7cc6c5c4)cc(c23)C1]

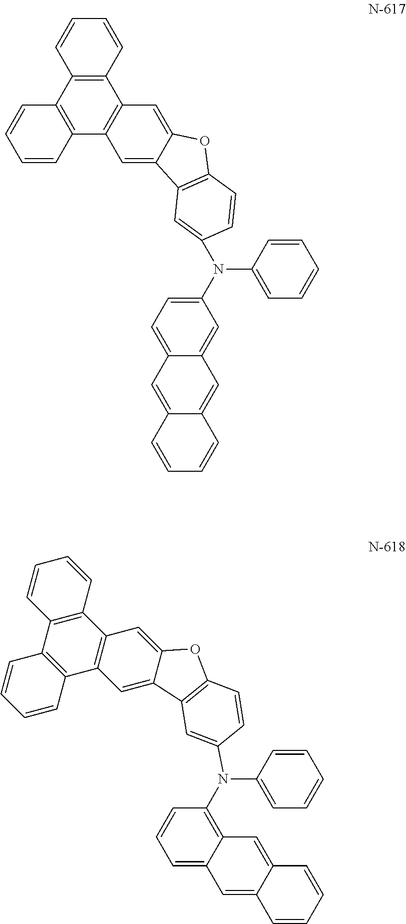 [SMILES: c1ccc(N(c2ccc3cc4ccccc4cc3c2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2cccc3cc4ccccc4cc23)cc1]

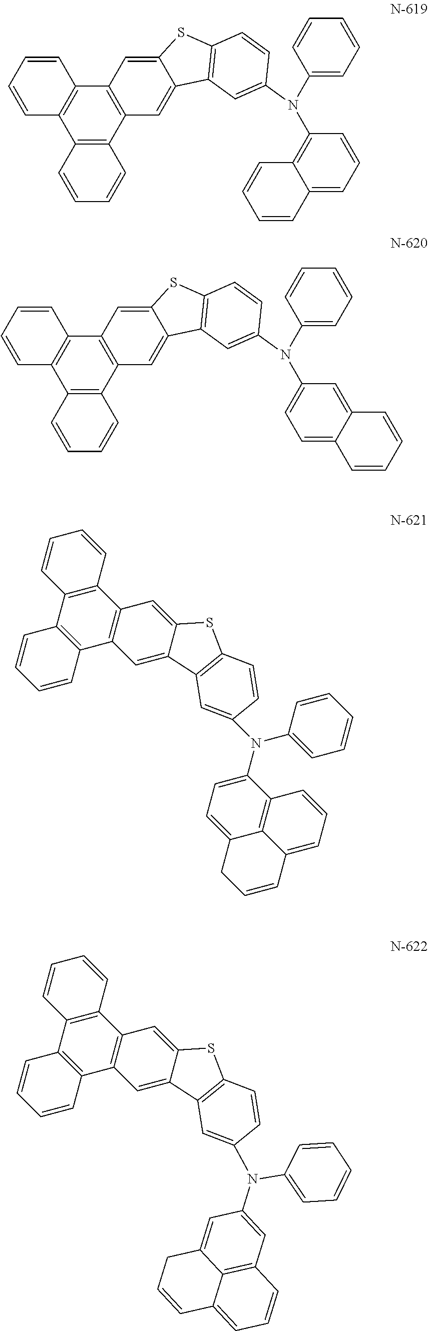 [SMILES: c1ccc(N(c2ccc3sc4cc5c6ccccc6c6ccccc6c5cc4c3c2)c2cccc3ccccc23)cc1 ; c1ccc(N(c2ccc3ccccc3c2)c2ccc3sc4cc5c6ccccc6c6ccccc6c5cc4c3c2)cc1 ; C1=Cc2cccc3c(N(c4ccccc4)c4ccc5sc6cc7c8ccccc8c8ccccc8c7cc6c5c4)ccc(c23)C1 ; C1=Cc2cccc3cc(N(c4ccccc4)c4ccc5sc6cc7c8ccccc8c8ccccc8c7cc6c5c4)cc(c23)C1]

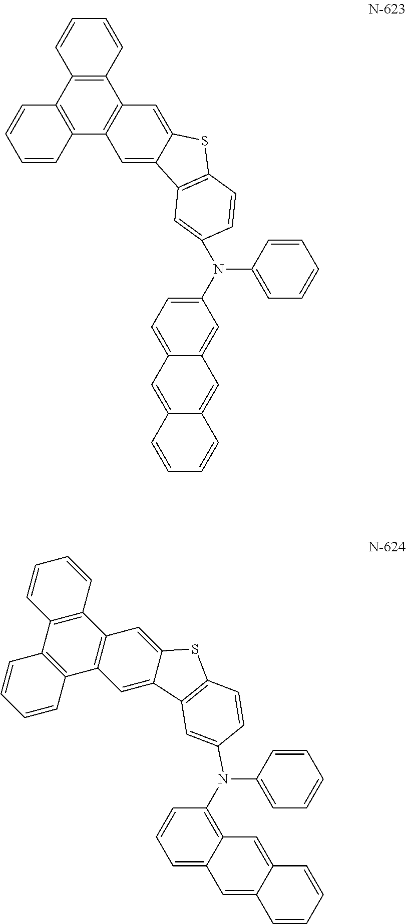 [SMILES: c1ccc(N(c2ccc3cc4ccccc4cc3c2)c2ccc3sc4cc5c6ccccc6c6ccccc6c5cc4c3c2)cc1 ; c1ccc(N(c2ccc3sc4cc5c6ccccc6c6ccccc6c5cc4c3c2)c2cccc3cc4ccccc4cc23)cc1]

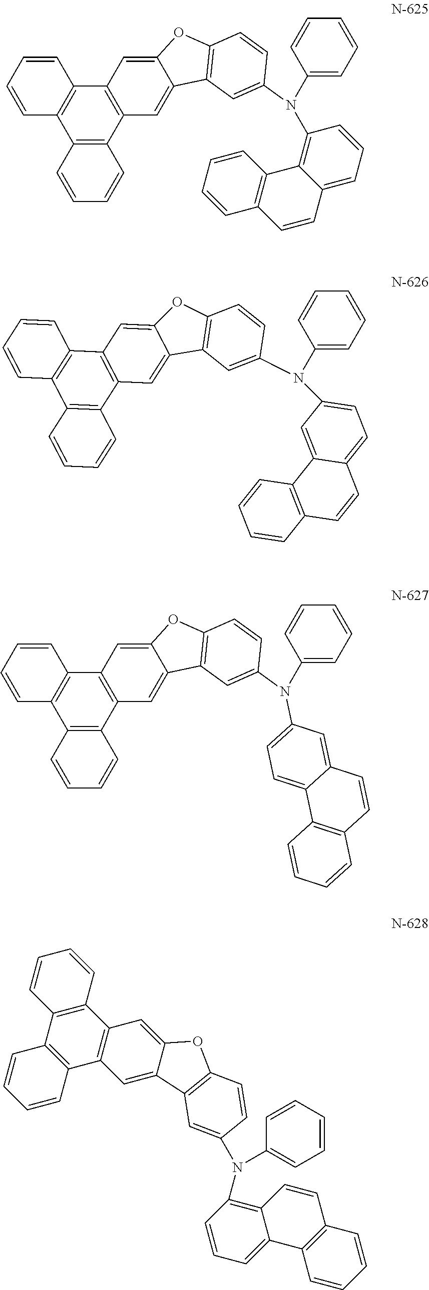 [SMILES: c1ccc(N(c2ccc3oc4cc5c6ccccc6c6ccccc6c5cc4c3c2)c2cccc3ccc4ccccc4c23)cc1 ; c1ccc(N(c2ccc3ccc4ccccc4c3c2)c2ccc3oc4cc5c6ccccc6c6ccccc6c5cc4c3c2)cc1 ; c1ccc(N(c2ccc3c(ccc4ccccc43)c2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2cccc3c2ccc2ccccc23)cc1]

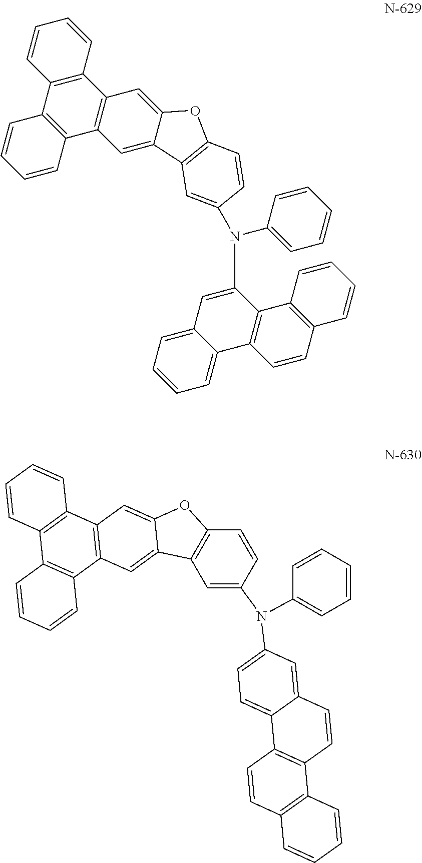 [SMILES: c1ccc(N(c2ccc3oc4cc5c6ccccc6c6ccccc6c5cc4c3c2)c2cc3ccccc3c3ccc4ccccc4c23)cc1 ; c1ccc(N(c2ccc3c(ccc4c5ccccc5ccc34)c2)c2ccc3oc4cc5c6ccccc6c6ccccc6c5cc4c3c2)cc1]

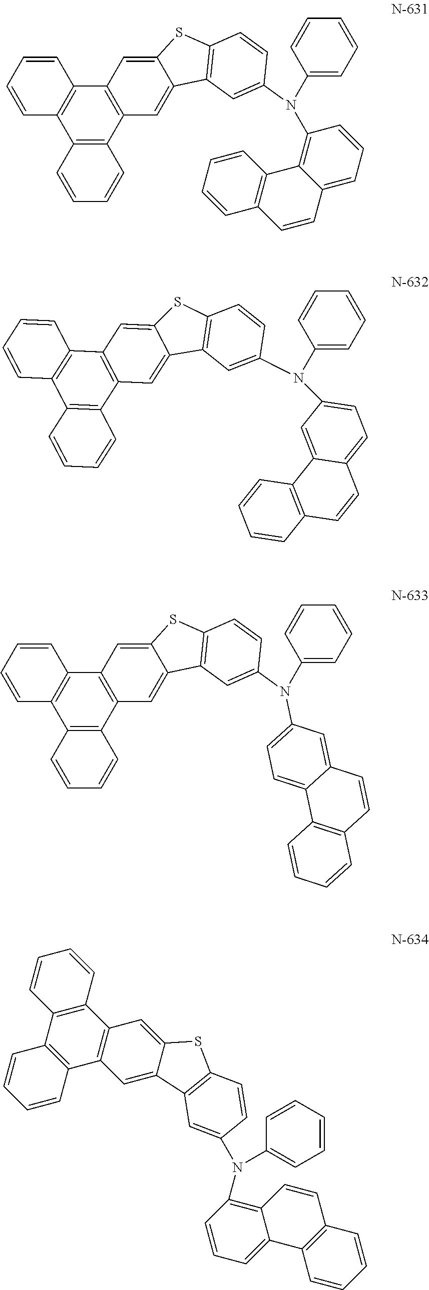 [SMILES: c1ccc(N(c2ccc3sc4cc5c6ccccc6c6ccccc6c5cc4c3c2)c2cccc3ccc4ccccc4c23)cc1 ; c1ccc(N(c2ccc3ccc4ccccc4c3c2)c2ccc3sc4cc5c6ccccc6c6ccccc6c5cc4c3c2)cc1 ; c1ccc(N(c2ccc3c(ccc4ccccc43)c2)c2ccc3sc4cc5c6ccccc6c6ccccc6c5cc4c3c2)cc1 ; c1ccc(N(c2ccc3sc4cc5c6ccccc6c6ccccc6c5cc4c3c2)c2cccc3c2ccc2ccccc23)cc1]

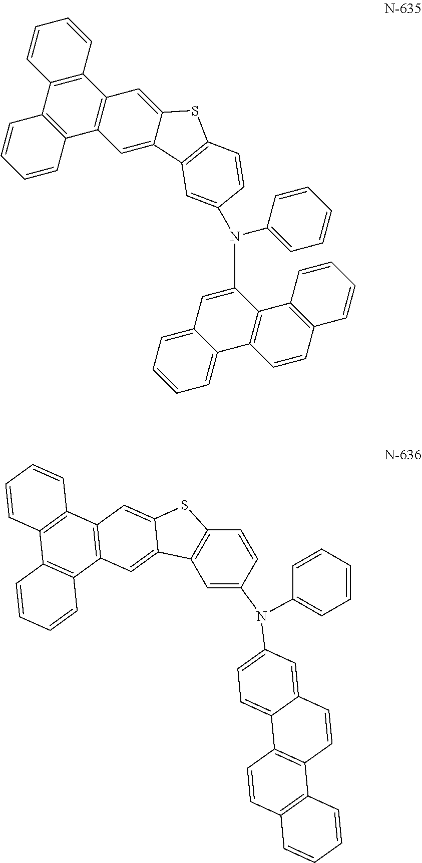 [SMILES: c1ccc(N(c2ccc3sc4cc5c6ccccc6c6ccccc6c5cc4c3c2)c2cc3ccccc3c3ccc4ccccc4c23)cc1 ; c1ccc(N(c2ccc3c(ccc4c5ccccc5ccc34)c2)c2ccc3sc4cc5c6ccccc6c6ccccc6c5cc4c3c2)cc1]

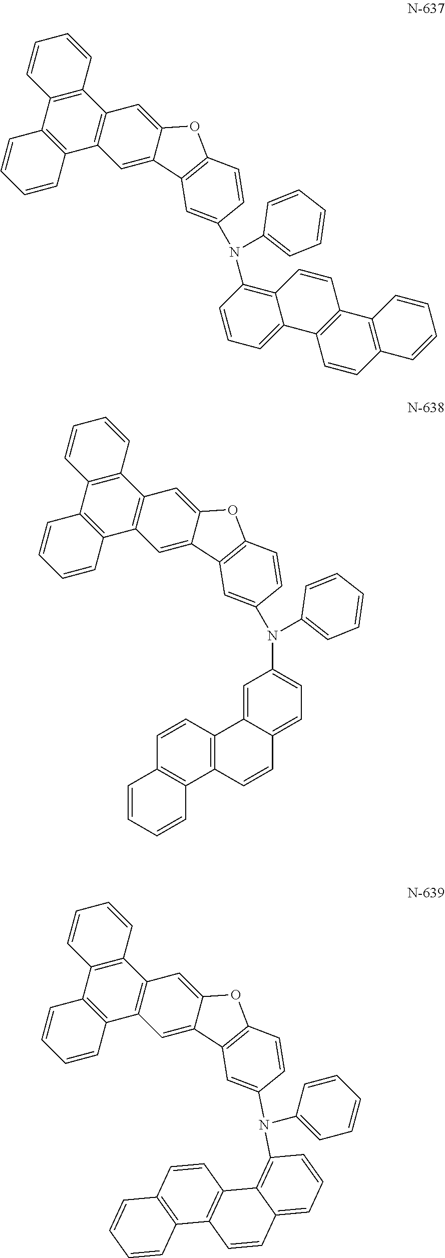 [SMILES: c1ccc(N(c2ccc3oc4cc5c6ccccc6c6ccccc6c5cc4c3c2)c2cccc3c2ccc2c4ccccc4ccc32)cc1 ; c1ccc(N(c2ccc3ccc4c5ccccc5ccc4c3c2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2cccc3ccc4c5ccccc5ccc4c23)cc1]

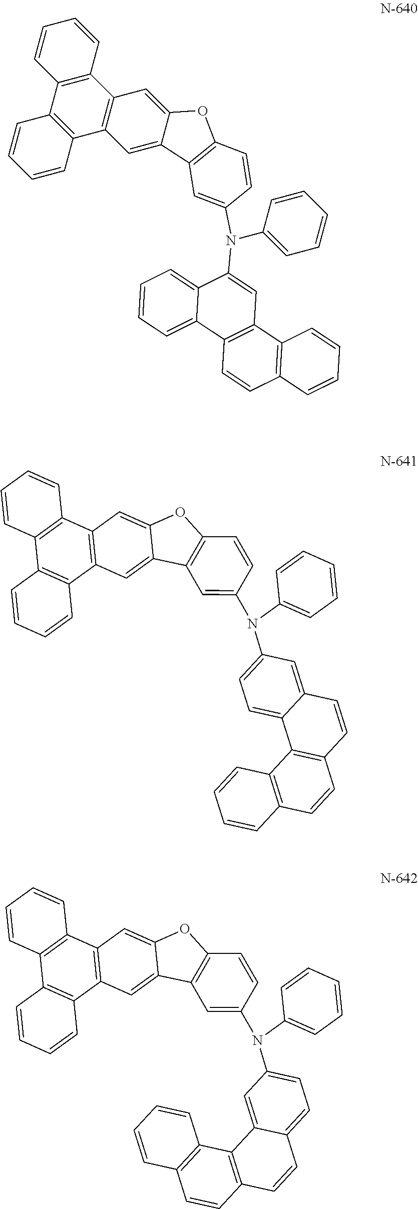 [SMILES: c1ccc(N(c2ccc3oc4cc5c6ccccc6c6ccccc6c5cc4c3c2)c2cc3c4ccccc4ccc3c3ccccc23)cc1 ; c1ccc(N(c2ccc3c(ccc4ccc5ccccc5c43)c2)c2ccc3oc4cc5c6ccccc6c6ccccc6c5cc4c3c2)cc1 ; c1ccc(N(c2ccc3oc4cc5c6ccccc6c6ccccc6c5cc4c3c2)c2ccc3ccc4ccc5ccccc5c4c3c2)cc1]

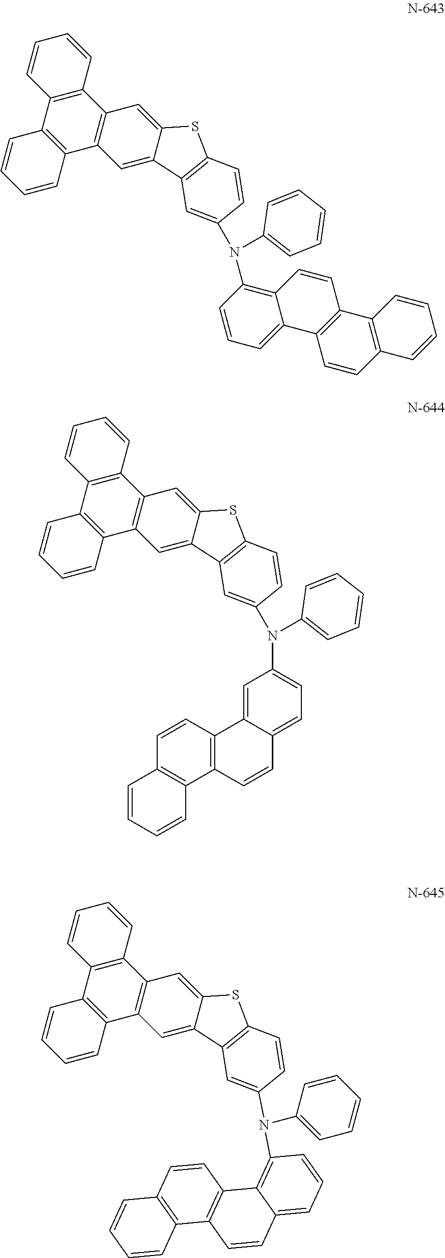 [SMILES: c1ccc(N(c2ccc3sc4cc5c6ccccc6c6ccccc6c5cc4c3c2)c2cccc3c2ccc2c4ccccc4ccc32)cc1 ; c1ccc(N(c2ccc3ccc4c5ccccc5ccc4c3c2)c2ccc3sc4cc5c6ccccc6c6ccccc6c5cc4c3c2)cc1 ; c1ccc(N(c2ccc3sc4cc5c6ccccc6c6ccccc6c5cc4c3c2)c2cccc3ccc4c5ccccc5ccc4c23)cc1]

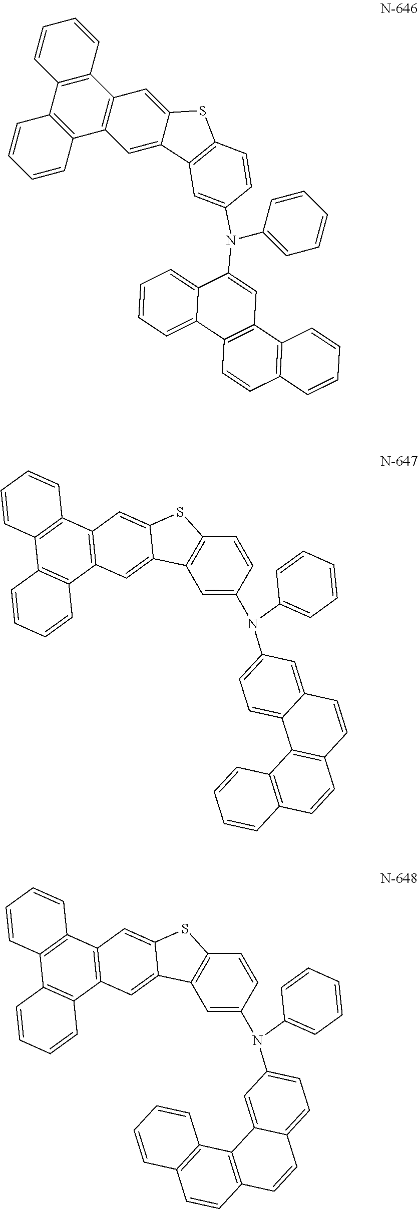 [SMILES: c1ccc(N(c2ccc3sc4cc5c6ccccc6c6ccccc6c5cc4c3c2)c2cc3c4ccccc4ccc3c3ccccc23)cc1 ; c1ccc(N(c2ccc3c(ccc4ccc5ccccc5c43)c2)c2ccc3sc4cc5c6ccccc6c6ccccc6c5cc4c3c2)cc1 ; c1ccc(N(c2ccc3sc4cc5c6ccccc6c6ccccc6c5cc4c3c2)c2ccc3ccc4ccc5ccccc5c4c3c2)cc1]

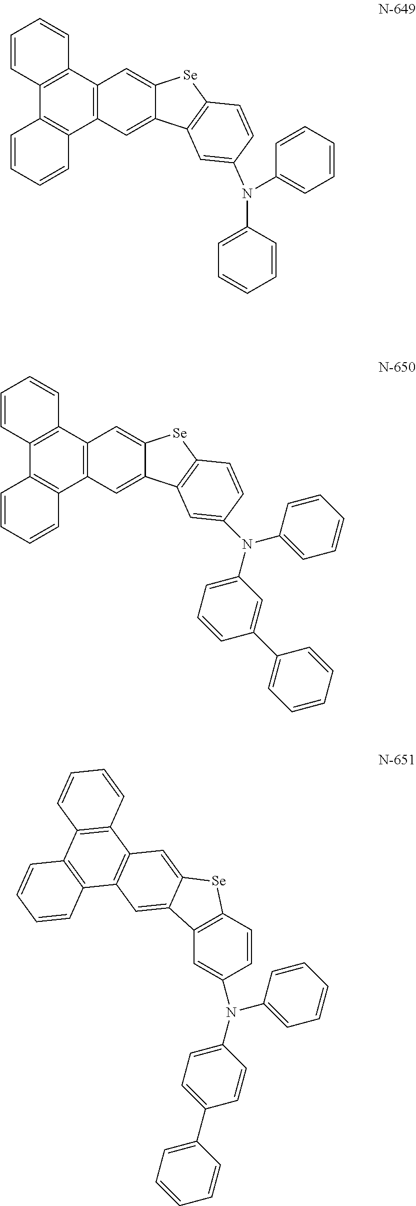 [SMILES: c1ccc(N(c2ccccc2)c2ccc3[se]c4cc5c6ccccc6c6ccccc6c5cc4c3c2)cc1 ; c1ccc(-c2cccc(N(c3ccccc3)c3ccc4[se]c5cc6c7ccccc7c7ccccc7c6cc5c4c3)c2)cc1 ; c1ccc(-c2ccc(N(c3ccccc3)c3ccc4[se]c5cc6c7ccccc7c7ccccc7c6cc5c4c3)cc2)cc1]

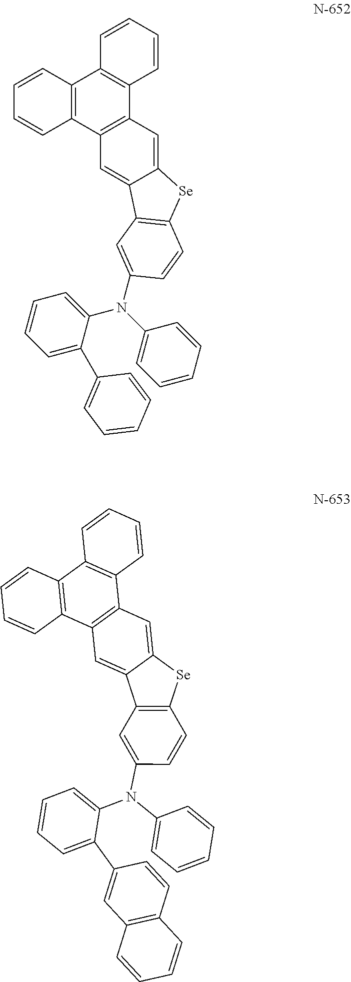 [SMILES: c1ccc(-c2ccccc2N(c2ccccc2)c2ccc3[se]c4cc5c6ccccc6c6ccccc6c5cc4c3c2)cc1 ; c1ccc(N(c2ccc3[se]c4cc5c6ccccc6c6ccccc6c5cc4c3c2)c2ccccc2-c2ccc3ccccc3c2)cc1]

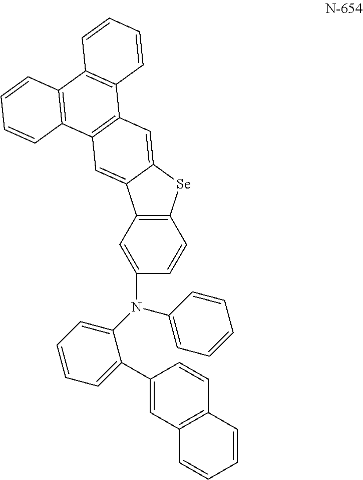 [SMILES: c1ccc(N(c2ccc3[se]c4cc5c6ccccc6c6ccccc6c5cc4c3c2)c2ccccc2-c2ccc3ccccc3c2)cc1]

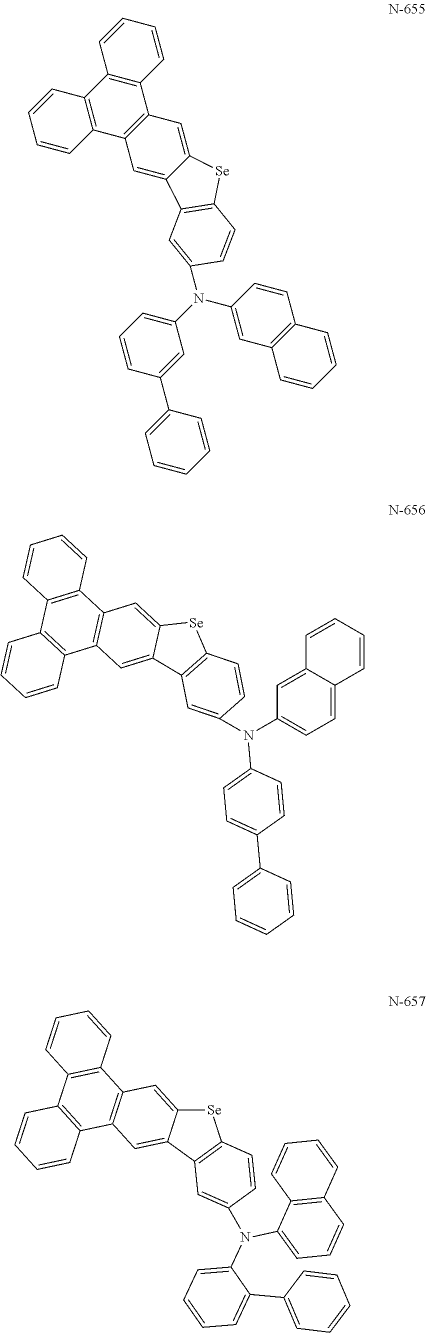 [SMILES: c1ccc(-c2cccc(N(c3ccc4ccccc4c3)c3ccc4[se]c5cc6c7ccccc7c7ccccc7c6cc5c4c3)c2)cc1 ; c1ccc(-c2ccc(N(c3ccc4ccccc4c3)c3ccc4[se]c5cc6c7ccccc7c7ccccc7c6cc5c4c3)cc2)cc1 ; c1ccc(-c2ccccc2N(c2ccc3[se]c4cc5c6ccccc6c6ccccc6c5cc4c3c2)c2cccc3ccccc23)cc1]

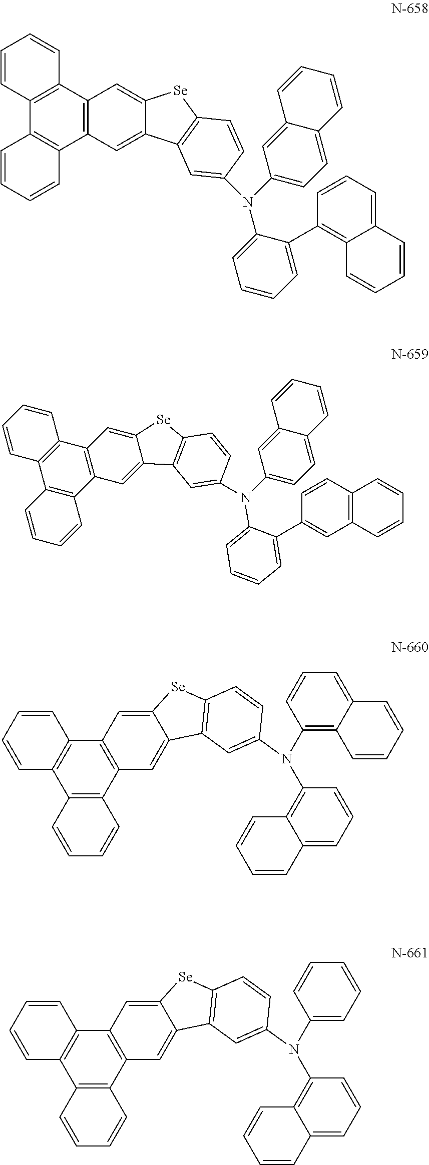 [SMILES: c1ccc(N(c2ccc3ccccc3c2)c2ccc3[se]c4cc5c6ccccc6c6ccccc6c5cc4c3c2)c(-c2cccc3ccccc23)c1 ; c1ccc(N(c2ccc3ccccc3c2)c2ccc3[se]c4cc5c6ccccc6c6ccccc6c5cc4c3c2)c(-c2ccc3ccccc3c2)c1 ; c1ccc2c(N(c3ccc4[se]c5cc6c7ccccc7c7ccccc7c6cc5c4c3)c3cccc4ccccc34)cccc2c1 ; c1ccc(N(c2ccc3[se]c4cc5c6ccccc6c6ccccc6c5cc4c3c2)c2cccc3ccccc23)cc1]

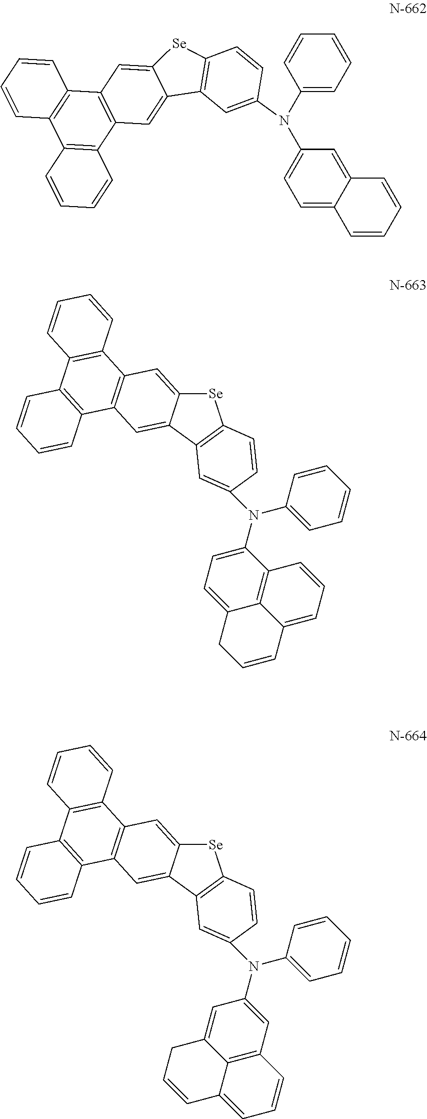 [SMILES: c1ccc(N(c2ccc3ccccc3c2)c2ccc3[se]c4cc5c6ccccc6c6ccccc6c5cc4c3c2)cc1 ; C1=Cc2cccc3c(N(c4ccccc4)c4ccc5[se]c6cc7c8ccccc8c8ccccc8c7cc6c5c4)ccc(c23)C1 ; C1=Cc2cccc3cc(N(c4ccccc4)c4ccc5[se]c6cc7c8ccccc8c8ccccc8c7cc6c5c4)cc(c23)C1]

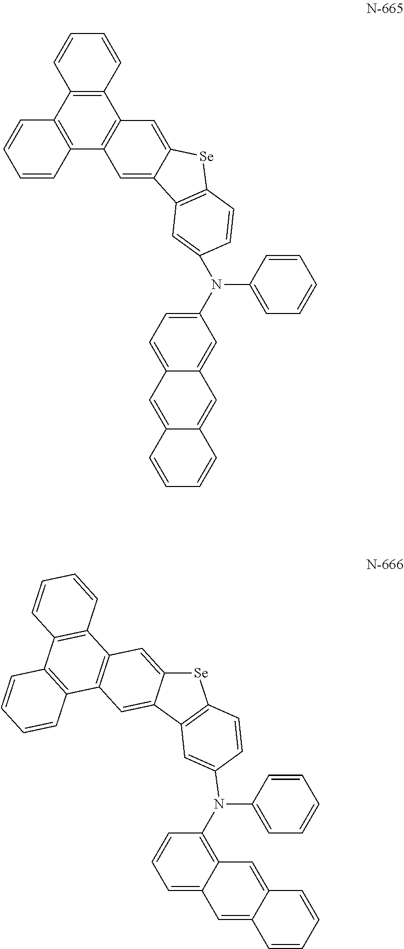 [SMILES: c1ccc(N(c2ccc3cc4ccccc4cc3c2)c2ccc3[se]c4cc5c6ccccc6c6ccccc6c5cc4c3c2)cc1 ; c1ccc(N(c2ccc3[se]c4cc5c6ccccc6c6ccccc6c5cc4c3c2)c2cccc3cc4ccccc4cc23)cc1]

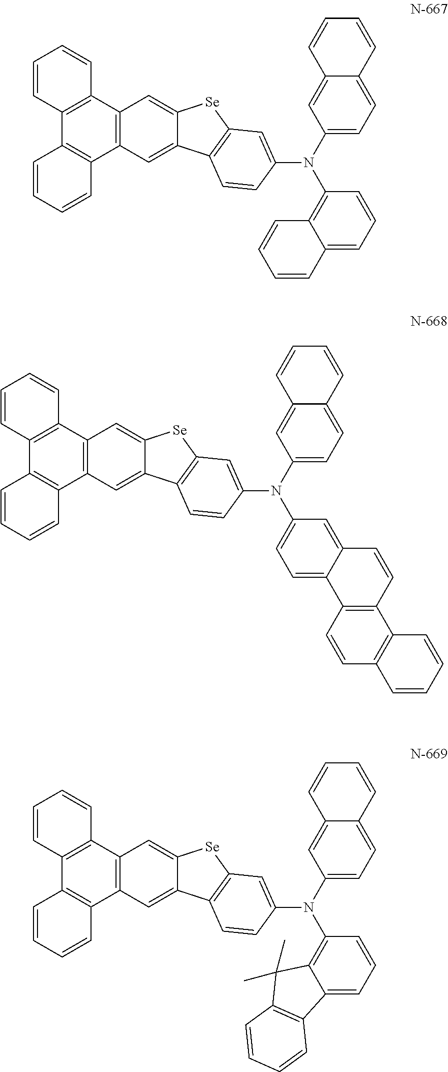 [SMILES: c1ccc2cc(N(c3ccc4c(c3)[se]c3cc5c6ccccc6c6ccccc6c5cc34)c3cccc4ccccc34)ccc2c1 ; c1ccc2cc(N(c3ccc4c(ccc5c6ccccc6ccc45)c3)c3ccc4c(c3)[se]c3cc5c6ccccc6c6ccccc6c5cc34)ccc2c1 ; CC1(C)c2ccccc2-c2cccc(N(c3ccc4ccccc4c3)c3ccc4c(c3)[se]c3cc5c6ccccc6c6ccccc6c5cc34)c21]

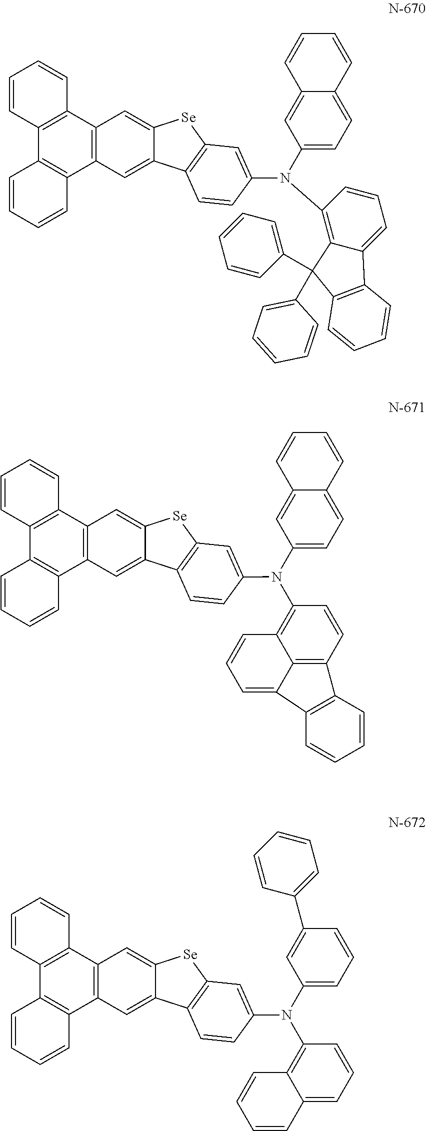 [SMILES: c1ccc(C2(c3ccccc3)c3ccccc3-c3cccc(N(c4ccc5ccccc5c4)c4ccc5c(c4)[se]c4cc6c7ccccc7c7ccccc7c6cc45)c32)cc1 ; c1ccc2c(c1)-c1cccc3c(N(c4ccc5ccccc5c4)c4ccc5c(c4)[se]c4cc6c7ccccc7c7ccccc7c6cc45)ccc-2c13 ; c1ccc(-c2cccc(N(c3ccc4c(c3)[se]c3cc5c6ccccc6c6ccccc6c5cc34)c3cccc4ccccc34)c2)cc1]

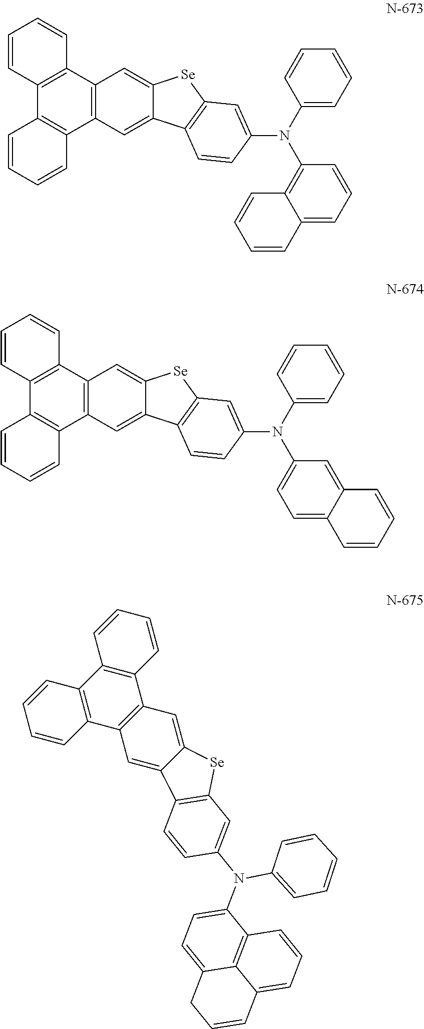 [SMILES: c1ccc(N(c2ccc3c(c2)[se]c2cc4c5ccccc5c5ccccc5c4cc23)c2cccc3ccccc23)cc1 ; c1ccc(N(c2ccc3ccccc3c2)c2ccc3c(c2)[se]c2cc4c5ccccc5c5ccccc5c4cc23)cc1 ; C1=Cc2cccc3c(N(c4ccccc4)c4ccc5c(c4)[se]c4cc6c7ccccc7c7ccccc7c6cc45)ccc(c23)C1]

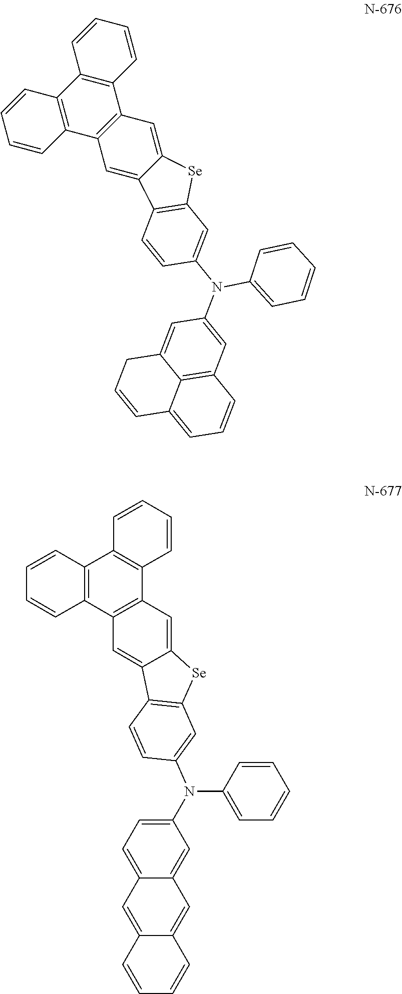 [SMILES: C1=Cc2cccc3cc(N(c4ccccc4)c4ccc5c(c4)[se]c4cc6c7ccccc7c7ccccc7c6cc45)cc(c23)C1 ; c1ccc(N(c2ccc3cc4ccccc4cc3c2)c2ccc3c(c2)[se]c2cc4c5ccccc5c5ccccc5c4cc23)cc1]

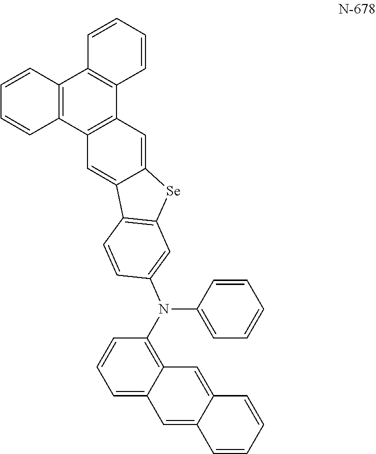 [SMILES: c1ccc(N(c2ccc3c(c2)[se]c2cc4c5ccccc5c5ccccc5c4cc23)c2cccc3cc4ccccc4cc23)cc1]

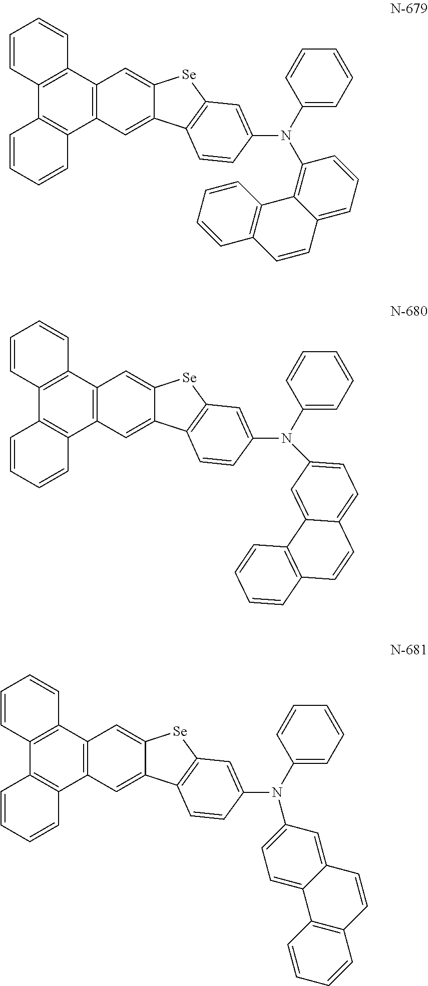 [SMILES: c1ccc(N(c2ccc3c(c2)[se]c2cc4c5ccccc5c5ccccc5c4cc23)c2cccc3ccc4ccccc4c23)cc1 ; c1ccc(N(c2ccc3c(c2)[se]c2cc4c5ccccc5c5ccccc5c4cc23)c2ccc3ccc4ccccc4c3c2)cc1 ; c1ccc(N(c2ccc3c(ccc4ccccc43)c2)c2ccc3c(c2)[se]c2cc4c5ccccc5c5ccccc5c4cc23)cc1]

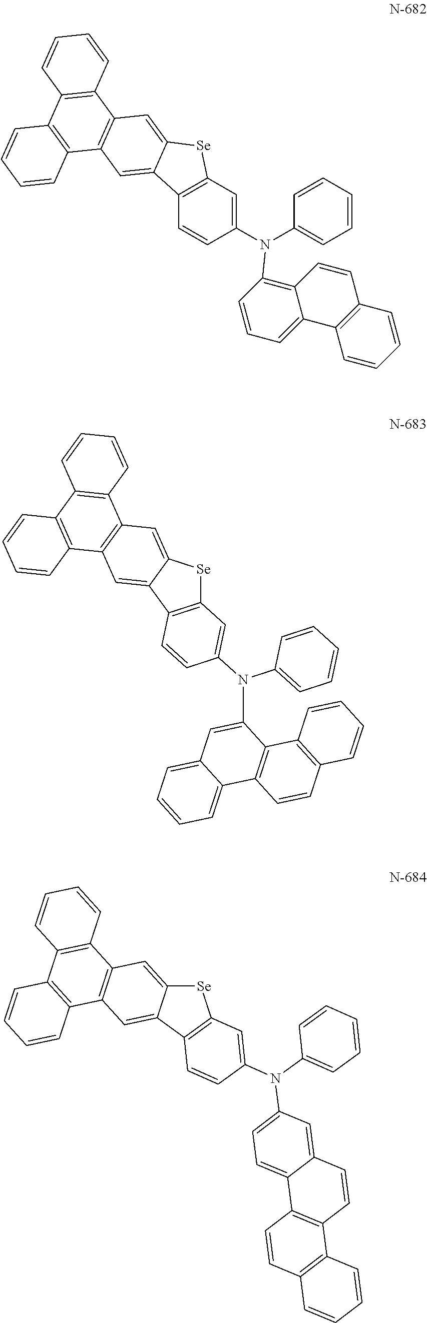 [SMILES: c1ccc(N(c2ccc3c(c2)[se]c2cc4c5ccccc5c5ccccc5c4cc23)c2cccc3c2ccc2ccccc23)cc1 ; c1ccc(N(c2ccc3c(c2)[se]c2cc4c5ccccc5c5ccccc5c4cc23)c2cc3ccccc3c3ccc4ccccc4c23)cc1 ; c1ccc(N(c2ccc3c(ccc4c5ccccc5ccc34)c2)c2ccc3c(c2)[se]c2cc4c5ccccc5c5ccccc5c4cc23)cc1]

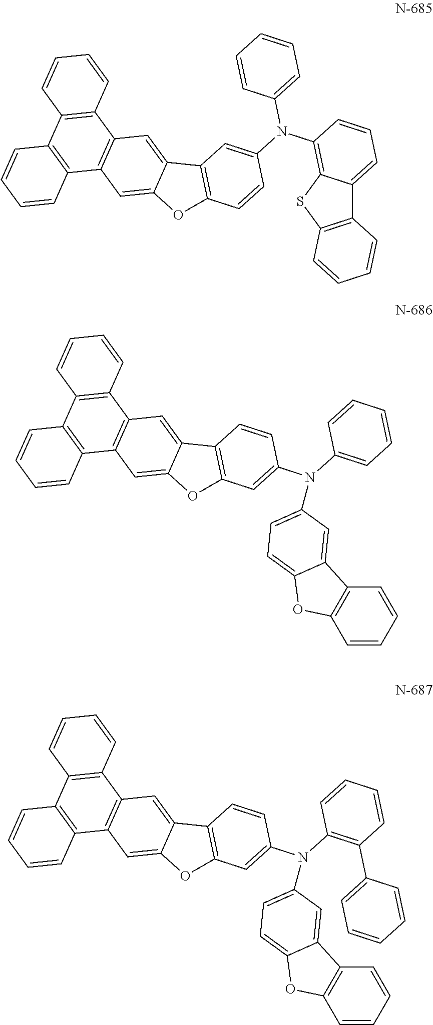 [SMILES: c1ccc(N(c2ccc3oc4cc5c6ccccc6c6ccccc6c5cc4c3c2)c2cccc3c2sc2ccccc23)cc1 ; c1ccc(N(c2ccc3c(c2)oc2cc4c5ccccc5c5ccccc5c4cc23)c2ccc3oc4ccccc4c3c2)cc1 ; c1ccc(-c2ccccc2N(c2ccc3c(c2)oc2cc4c5ccccc5c5ccccc5c4cc23)c2ccc3oc4ccccc4c3c2)cc1]

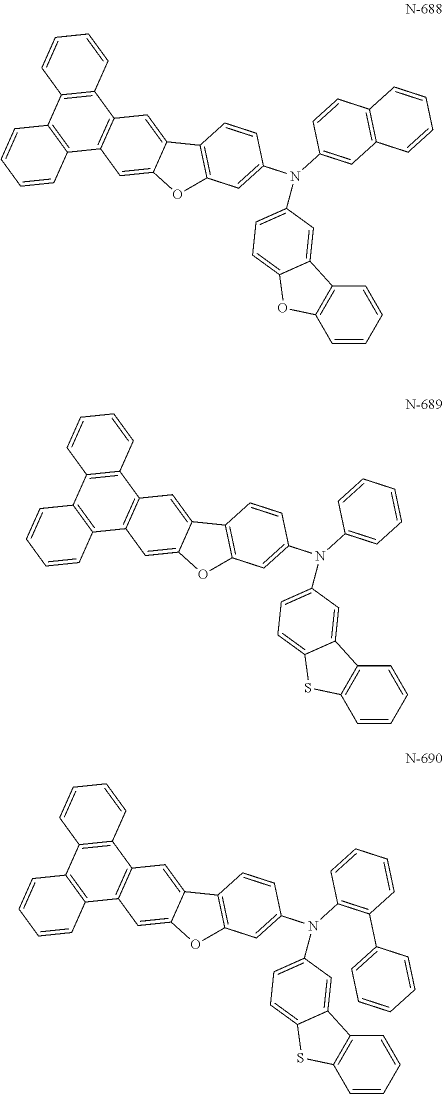 [SMILES: c1ccc2cc(N(c3ccc4c(c3)oc3cc5c6ccccc6c6ccccc6c5cc34)c3ccc4oc5ccccc5c4c3)ccc2c1 ; c1ccc(N(c2ccc3c(c2)oc2cc4c5ccccc5c5ccccc5c4cc23)c2ccc3sc4ccccc4c3c2)cc1 ; c1ccc(-c2ccccc2N(c2ccc3c(c2)oc2cc4c5ccccc5c5ccccc5c4cc23)c2ccc3sc4ccccc4c3c2)cc1]

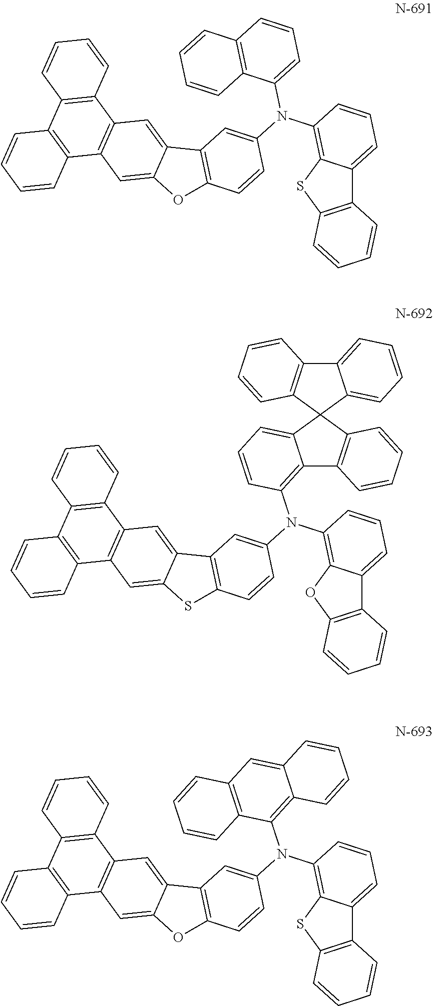 [SMILES: c1ccc2c(N(c3ccc4oc5cc6c7ccccc7c7ccccc7c6cc5c4c3)c3cccc4c3sc3ccccc34)cccc2c1 ; c1ccc2c(c1)-c1ccccc1C21c2ccccc2-c2c(N(c3ccc4sc5cc6c7ccccc7c7ccccc7c6cc5c4c3)c3cccc4c3oc3ccccc34)cccc21 ; c1ccc2c(N(c3ccc4oc5cc6c7ccccc7c7ccccc7c6cc5c4c3)c3cccc4c3sc3ccccc34)c3ccccc3cc2c1]

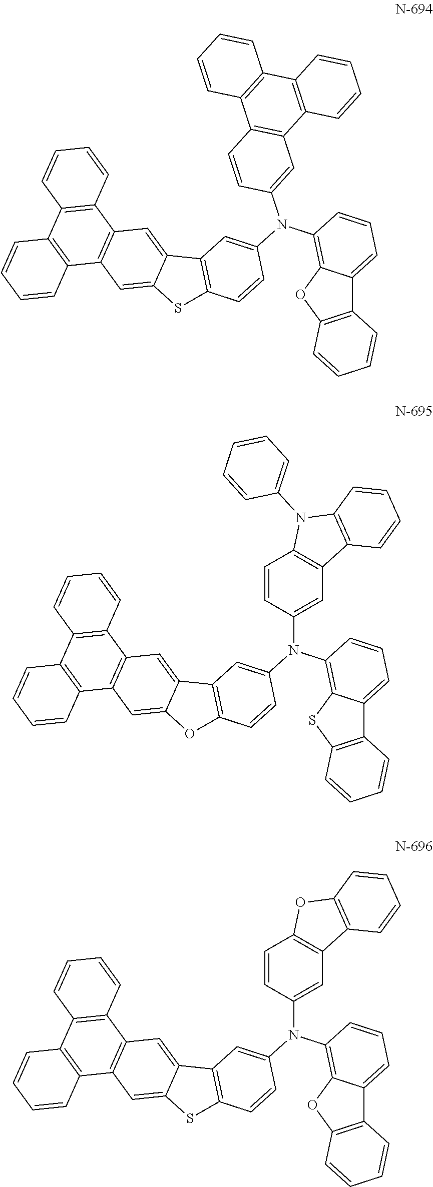 [SMILES: c1ccc2c(c1)oc1c(N(c3ccc4sc5cc6c7ccccc7c7ccccc7c6cc5c4c3)c3ccc4c5ccccc5c5ccccc5c4c3)cccc12 ; c1ccc(-n2c3ccccc3c3cc(N(c4ccc5oc6cc7c8ccccc8c8ccccc8c7cc6c5c4)c4cccc5c4sc4ccccc45)ccc32)cc1 ; c1ccc2c(c1)oc1ccc(N(c3ccc4sc5cc6c7ccccc7c7ccccc7c6cc5c4c3)c3cccc4c3oc3ccccc34)cc12]

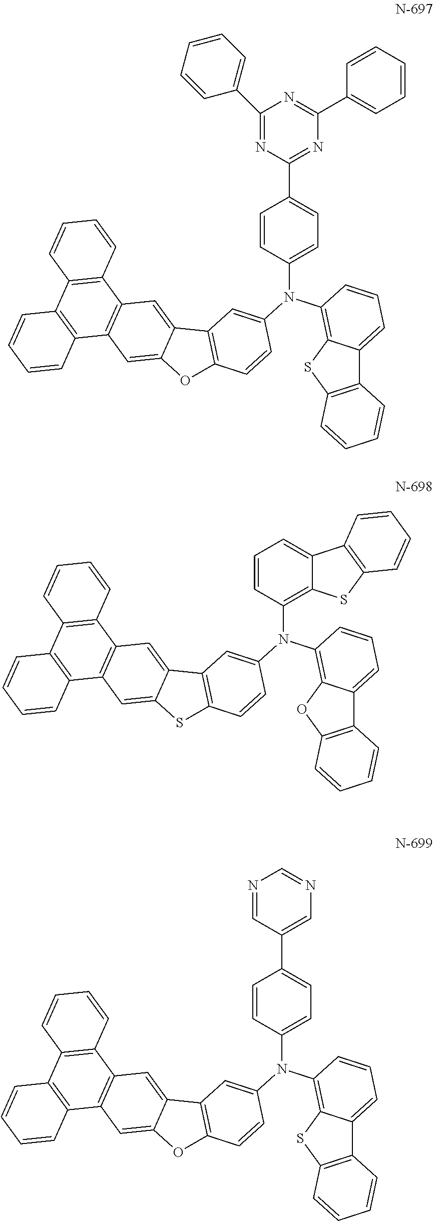 [SMILES: c1ccc(-c2nc(-c3ccccc3)nc(-c3ccc(N(c4ccc5oc6cc7c8ccccc8c8ccccc8c7cc6c5c4)c4cccc5c4sc4ccccc45)cc3)n2)cc1 ; c1ccc2c(c1)oc1c(N(c3ccc4sc5cc6c7ccccc7c7ccccc7c6cc5c4c3)c3cccc4c3sc3ccccc34)cccc12 ; c1ccc2c(c1)sc1c(N(c3ccc(-c4cncnc4)cc3)c3ccc4oc5cc6c7ccccc7c7ccccc7c6cc5c4c3)cccc12]

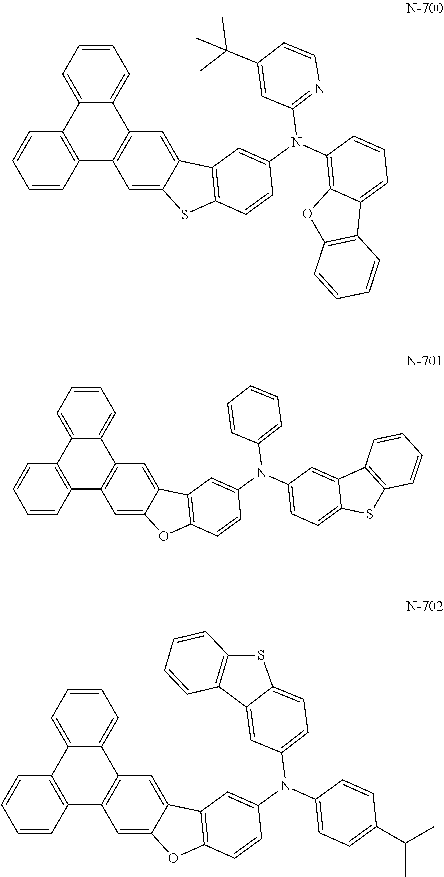 [SMILES: CC(C)(C)c1ccnc(N(c2ccc3sc4cc5c6ccccc6c6ccccc6c5cc4c3c2)c2cccc3c2oc2ccccc23)c1 ; c1ccc(N(c2ccc3oc4cc5c6ccccc6c6ccccc6c5cc4c3c2)c2ccc3sc4ccccc4c3c2)cc1 ; CC(C)c1ccc(N(c2ccc3oc4cc5c6ccccc6c6ccccc6c5cc4c3c2)c2ccc3sc4ccccc4c3c2)cc1]

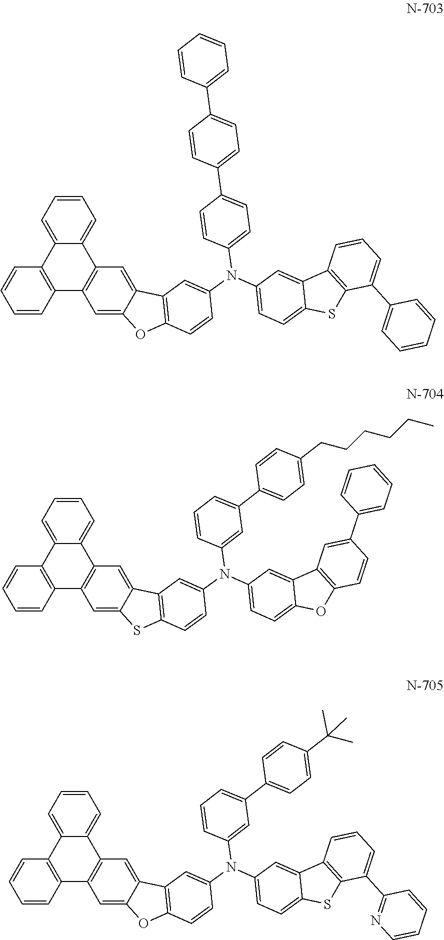 [SMILES: c1ccc(-c2ccc(-c3ccc(N(c4ccc5oc6cc7c8ccccc8c8ccccc8c7cc6c5c4)c4ccc5sc6c(-c7ccccc7)cccc6c5c4)cc3)cc2)cc1 ; CCCCCCc1ccc(-c2cccc(N(c3ccc4oc5ccc(-c6ccccc6)cc5c4c3)c3ccc4sc5cc6c7ccccc7c7ccccc7c6cc5c4c3)c2)cc1 ; CC(C)(C)c1ccc(-c2cccc(N(c3ccc4oc5cc6c7ccccc7c7ccccc7c6cc5c4c3)c3ccc4sc5c(-c6ccccn6)cccc5c4c3)c2)cc1]

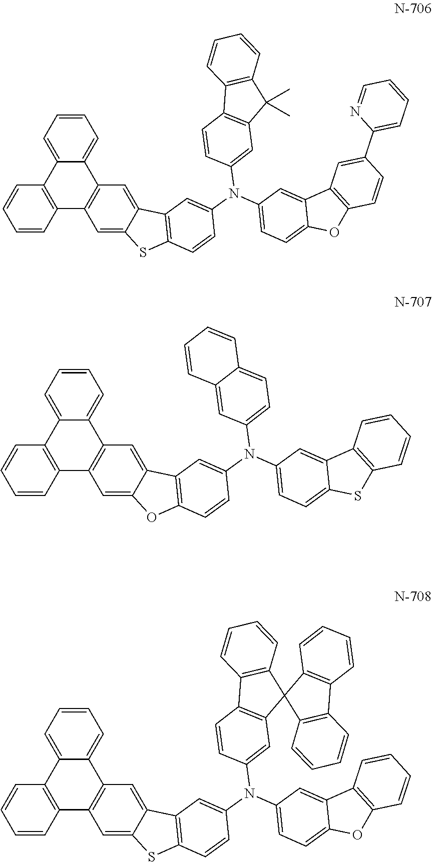 [SMILES: CC1(C)c2ccccc2-c2ccc(N(c3ccc4oc5ccc(-c6ccccn6)cc5c4c3)c3ccc4sc5cc6c7ccccc7c7ccccc7c6cc5c4c3)cc21 ; c1ccc2cc(N(c3ccc4oc5cc6c7ccccc7c7ccccc7c6cc5c4c3)c3ccc4sc5ccccc5c4c3)ccc2c1 ; c1ccc2c(c1)-c1ccccc1C21c2ccccc2-c2ccc(N(c3ccc4oc5ccccc5c4c3)c3ccc4sc5cc6c7ccccc7c7ccccc7c6cc5c4c3)cc21]

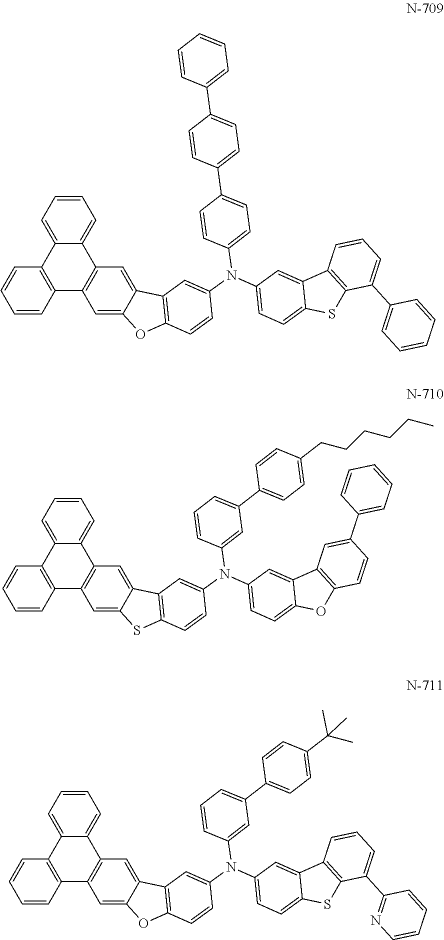 [SMILES: c1ccc(-c2ccc(-c3ccc(N(c4ccc5oc6cc7c8ccccc8c8ccccc8c7cc6c5c4)c4ccc5sc6c(-c7ccccc7)cccc6c5c4)cc3)cc2)cc1 ; CCCCCCc1ccc(-c2cccc(N(c3ccc4oc5ccc(-c6ccccc6)cc5c4c3)c3ccc4sc5cc6c7ccccc7c7ccccc7c6cc5c4c3)c2)cc1 ; CC(C)(C)c1ccc(-c2cccc(N(c3ccc4oc5cc6c7ccccc7c7ccccc7c6cc5c4c3)c3ccc4sc5c(-c6ccccn6)cccc5c4c3)c2)cc1]

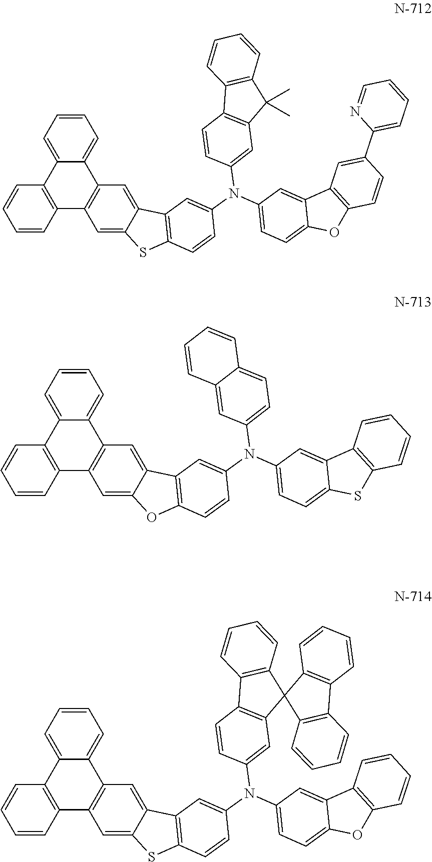 [SMILES: CC1(C)c2ccccc2-c2ccc(N(c3ccc4oc5ccc(-c6ccccn6)cc5c4c3)c3ccc4sc5cc6c7ccccc7c7ccccc7c6cc5c4c3)cc21 ; c1ccc2cc(N(c3ccc4oc5cc6c7ccccc7c7ccccc7c6cc5c4c3)c3ccc4sc5ccccc5c4c3)ccc2c1 ; c1ccc2c(c1)-c1ccccc1C21c2ccccc2-c2ccc(N(c3ccc4oc5ccccc5c4c3)c3ccc4sc5cc6c7ccccc7c7ccccc7c6cc5c4c3)cc21]

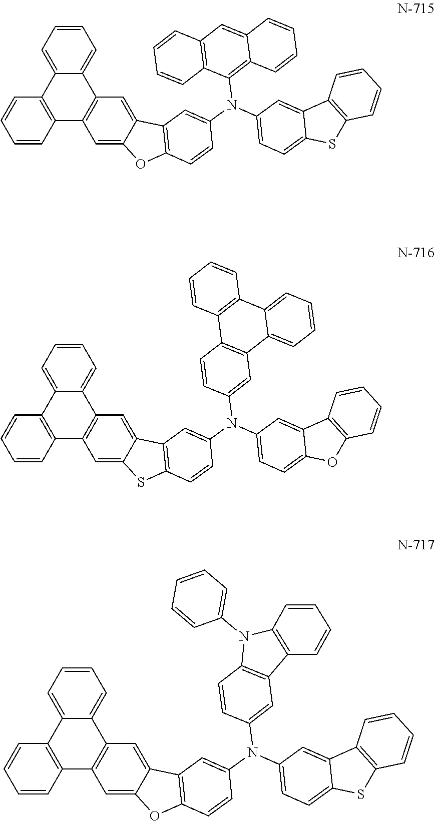 [SMILES: c1ccc2c(N(c3ccc4oc5cc6c7ccccc7c7ccccc7c6cc5c4c3)c3ccc4sc5ccccc5c4c3)c3ccccc3cc2c1 ; c1ccc2c(c1)oc1ccc(N(c3ccc4sc5cc6c7ccccc7c7ccccc7c6cc5c4c3)c3ccc4c5ccccc5c5ccccc5c4c3)cc12 ; c1ccc(-n2c3ccccc3c3cc(N(c4ccc5oc6cc7c8ccccc8c8ccccc8c7cc6c5c4)c4ccc5sc6ccccc6c5c4)ccc32)cc1]

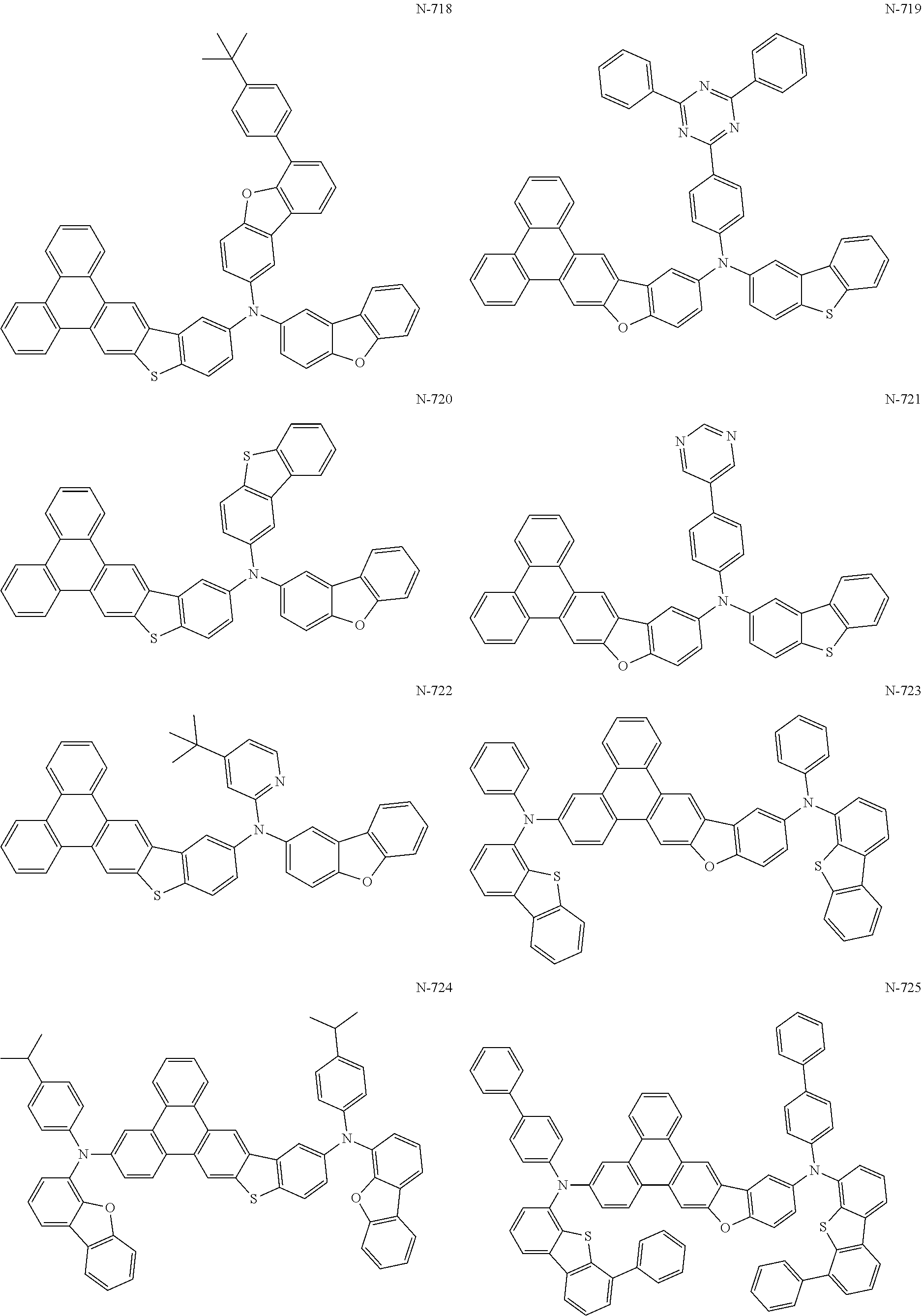

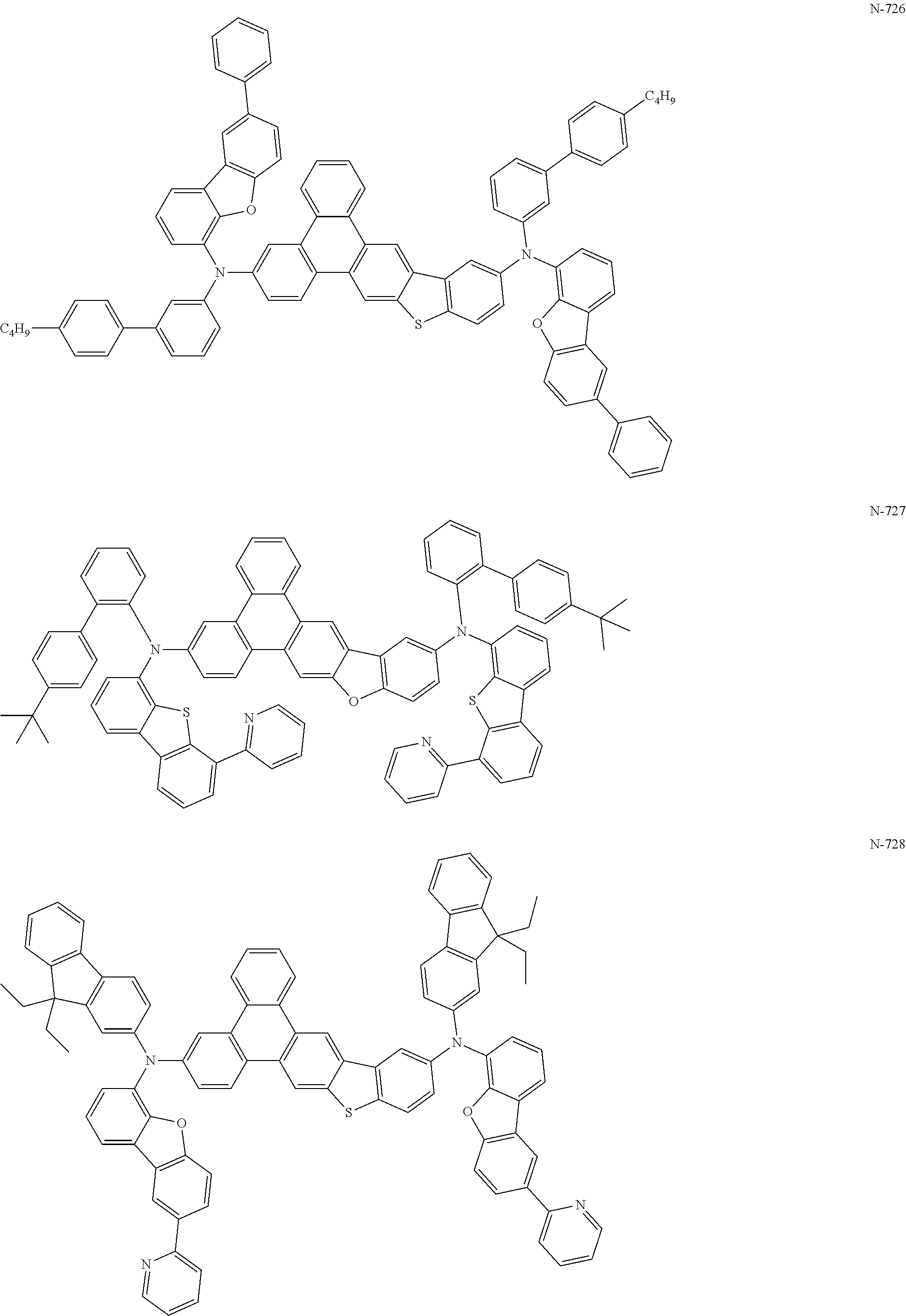

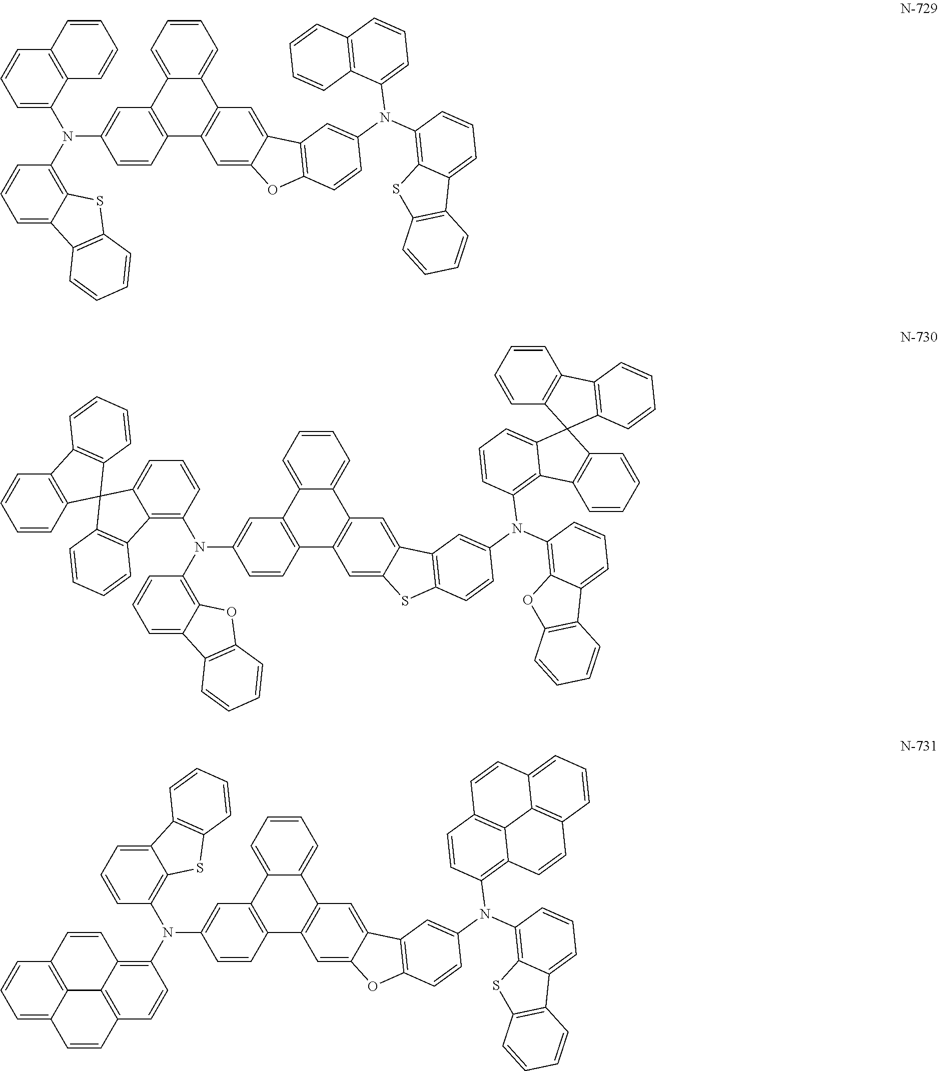 [SMILES: c1ccc2c(N(c3ccc4oc5cc6c7ccc(N(c8cccc9ccccc89)c8cccc9c8sc8ccccc89)cc7c7ccccc7c6cc5c4c3)c3cccc4c3sc3ccccc34)cccc2c1 ; c1ccc2c(c1)-c1ccccc1C21c2ccccc2-c2c(N(c3ccc4sc5cc6c7ccc(N(c8cccc9c8-c8ccccc8C98c9ccccc9-c9ccccc98)c8cccc9c8oc8ccccc89)cc7c7ccccc7c6cc5c4c3)c3cccc4c3oc3ccccc34)cccc21 ; c1cc2ccc3ccc(N(c4ccc5oc6cc7c8ccc(N(c9ccc%10ccc%11cccc%12ccc9c%10c%11%12)c9cccc%10c9sc9ccccc9%10)cc8c8ccccc8c7cc6c5c4)c4cccc5c4sc4ccccc45)c4ccc(c1)c2c34]

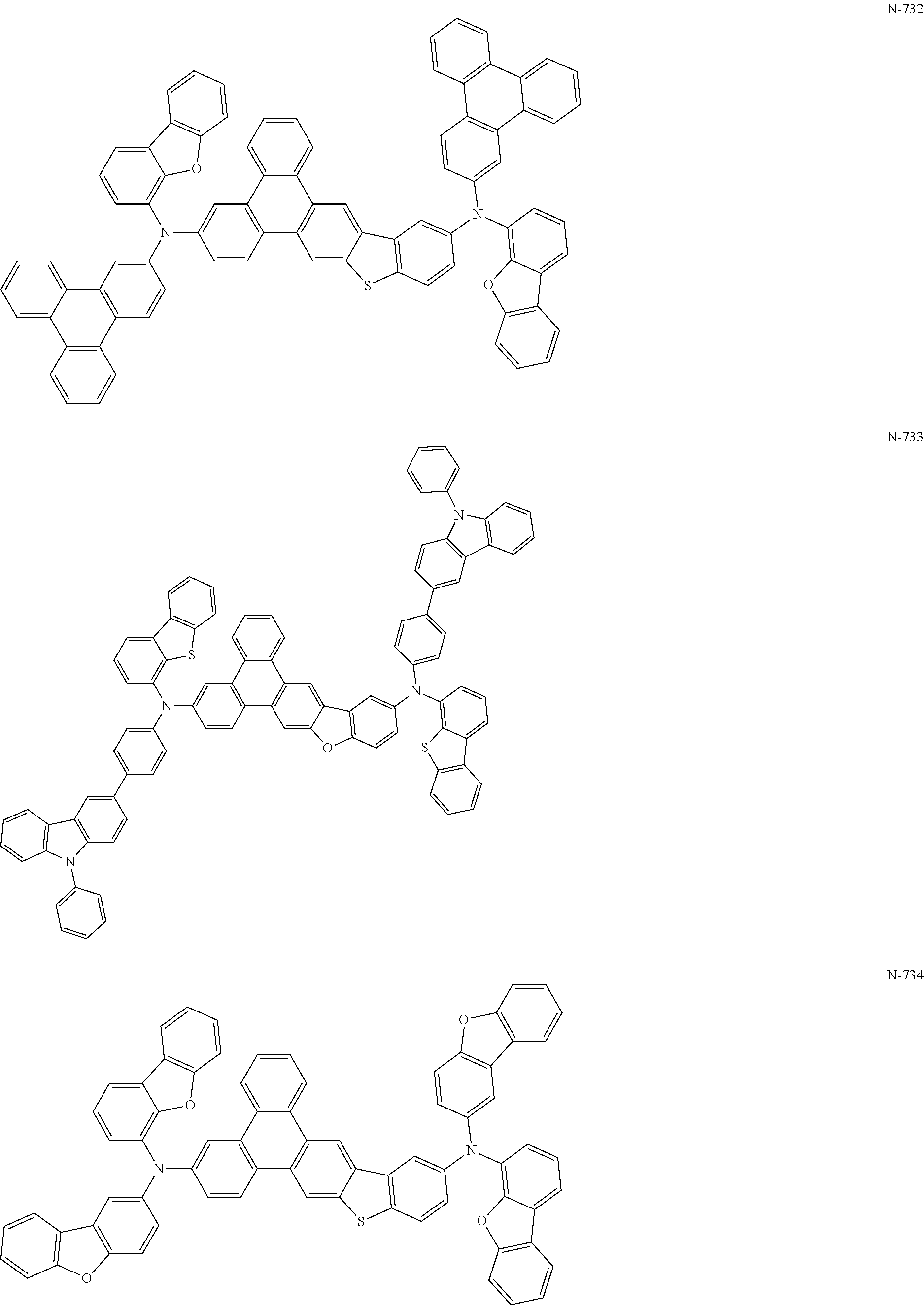 [SMILES: c1ccc2c(c1)oc1c(N(c3ccc4sc5cc6c7ccc(N(c8ccc9c%10ccccc%10c%10ccccc%10c9c8)c8cccc9c8oc8ccccc89)cc7c7ccccc7c6cc5c4c3)c3ccc4c5ccccc5c5ccccc5c4c3)cccc12 ; c1ccc(-n2c3ccccc3c3cc(-c4ccc(N(c5ccc6oc7cc8c9ccc(N(c%10ccc(-c%11ccc%12c(c%11)c%11ccccc%11n%12-c%11ccccc%11)cc%10)c%10cccc%11c%10sc%10ccccc%10%11)cc9c9ccccc9c8cc7c6c5)c5cccc6c5sc5ccccc56)cc4)ccc32)cc1 ; c1ccc2c(c1)oc1ccc(N(c3ccc4sc5cc6c7ccc(N(c8ccc9oc%10ccccc%10c9c8)c8cccc9c8oc8ccccc89)cc7c7ccccc7c6cc5c4c3)c3cccc4c3oc3ccccc34)cc12]

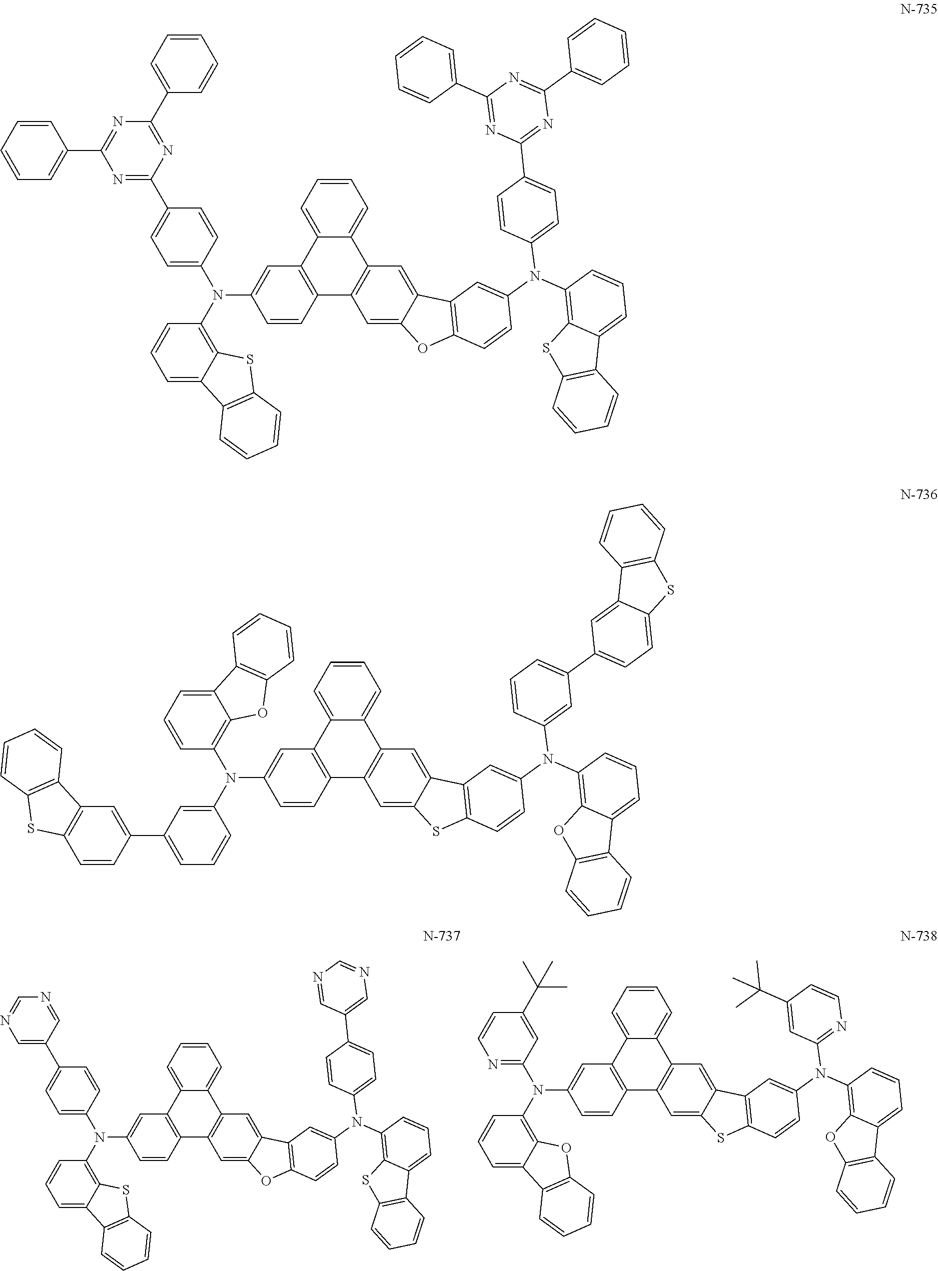

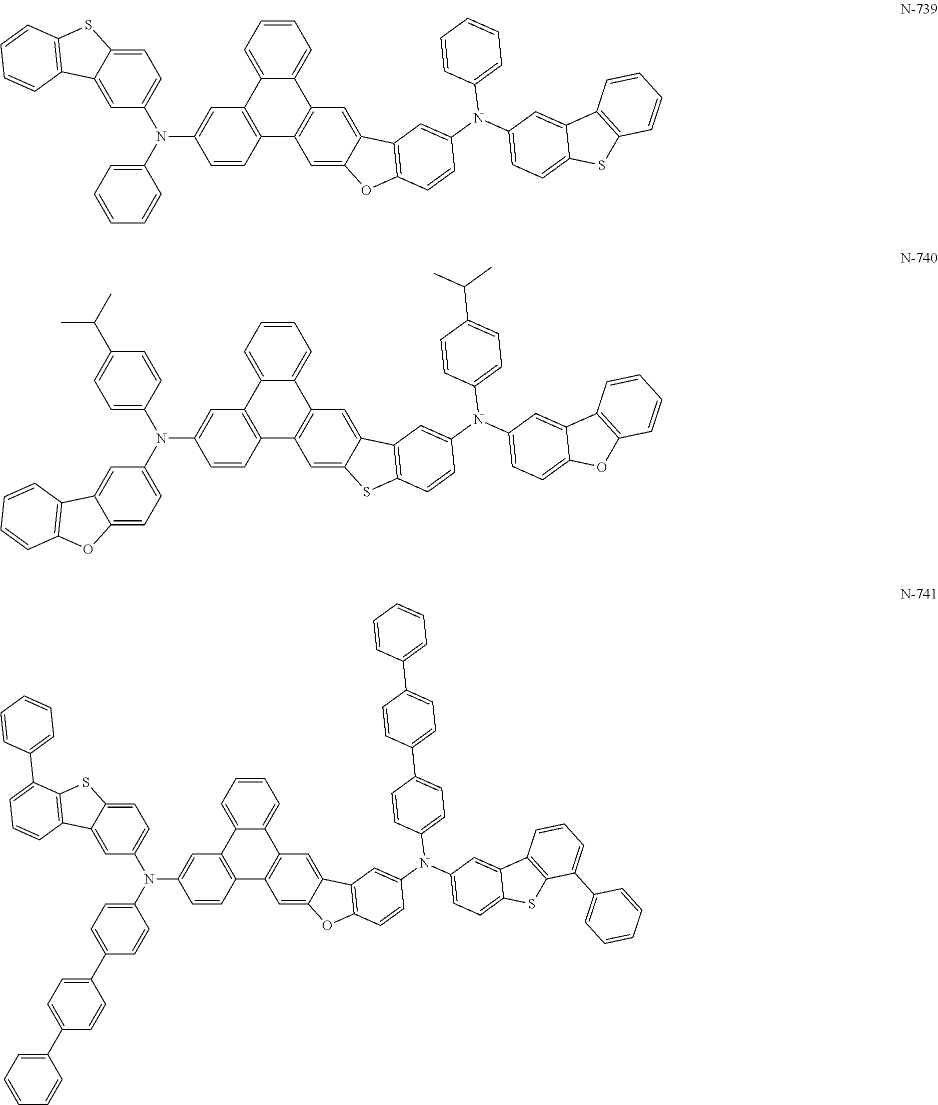 [SMILES: c1ccc(N(c2ccc3oc4cc5c6ccc(N(c7ccccc7)c7ccc8sc9ccccc9c8c7)cc6c6ccccc6c5cc4c3c2)c2ccc3sc4ccccc4c3c2)cc1 ; CC(C)c1ccc(N(c2ccc3oc4ccccc4c3c2)c2ccc3sc4cc5c6ccc(N(c7ccc(C(C)C)cc7)c7ccc8oc9ccccc9c8c7)cc6c6ccccc6c5cc4c3c2)cc1 ; c1ccc(-c2ccc(-c3ccc(N(c4ccc5oc6cc7c8ccc(N(c9ccc(-c%10ccc(-c%11ccccc%11)cc%10)cc9)c9ccc%10sc%11c(-c%12ccccc%12)cccc%11c%10c9)cc8c8ccccc8c7cc6c5c4)c4ccc5sc6c(-c7ccccc7)cccc6c5c4)cc3)cc2)cc1]

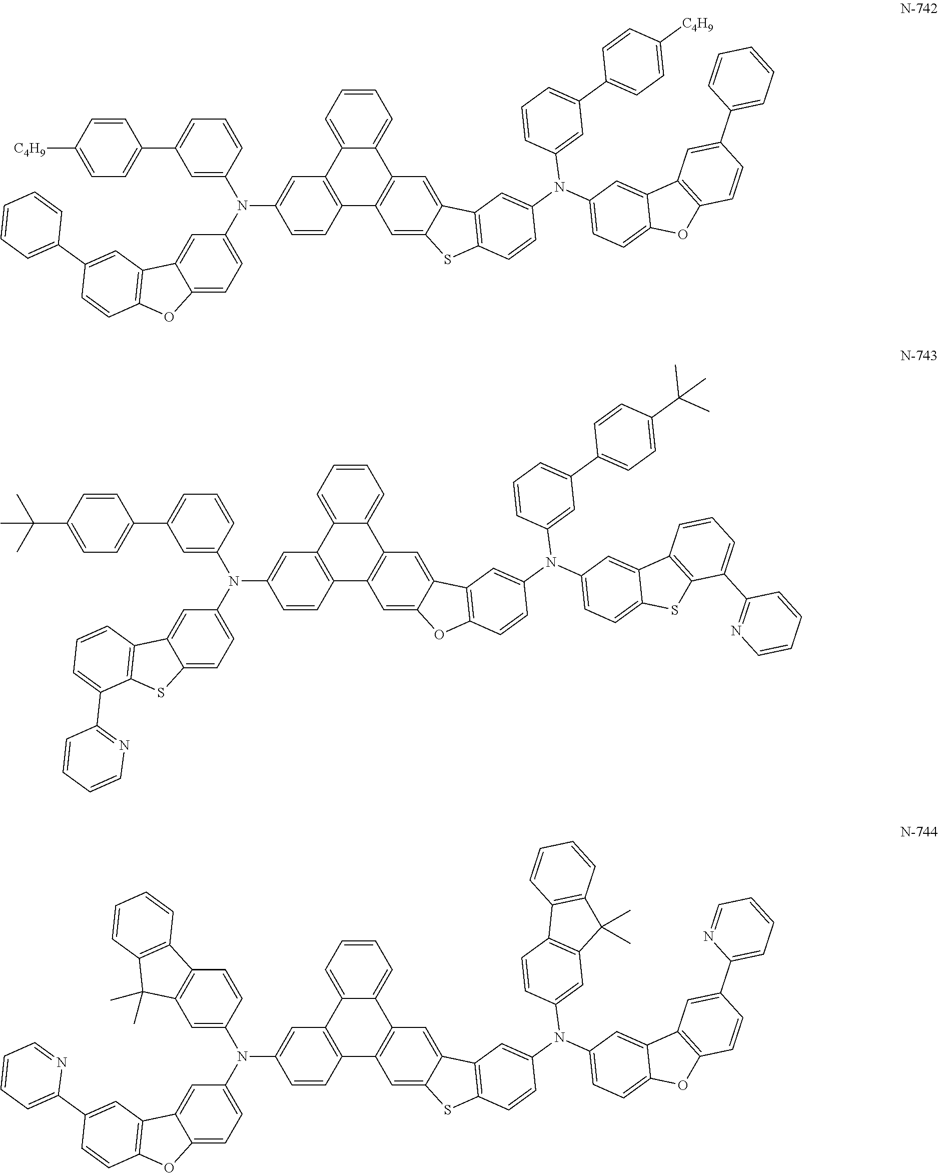

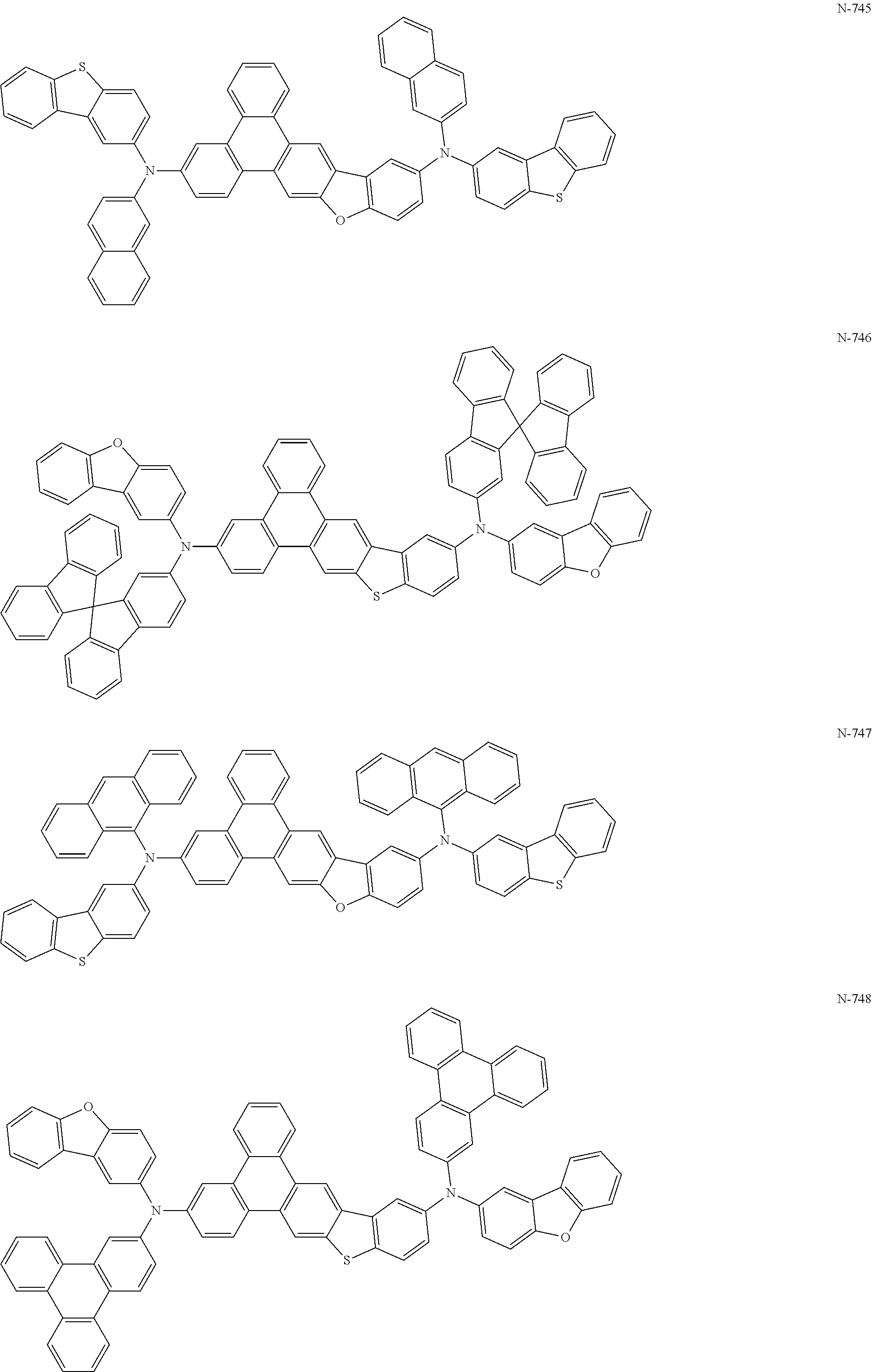

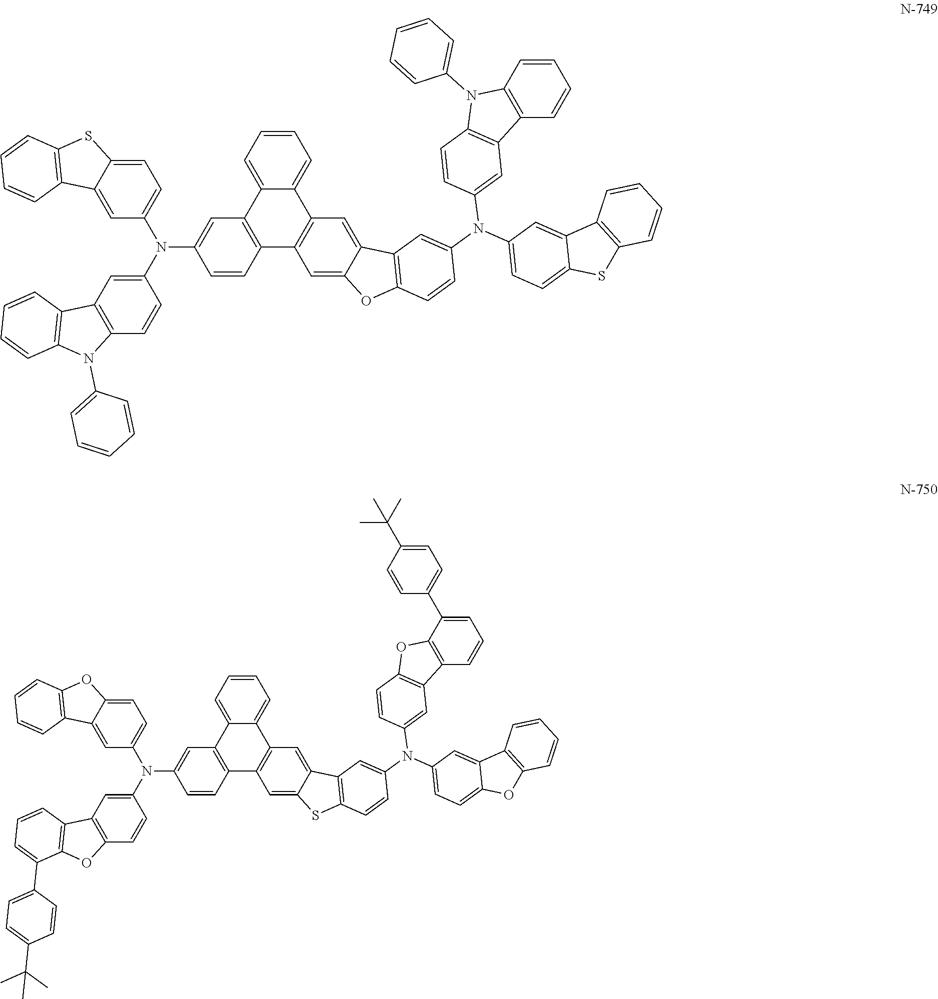 [SMILES: c1ccc(-n2c3ccccc3c3cc(N(c4ccc5oc6cc7c8ccc(N(c9ccc%10sc%11ccccc%11c%10c9)c9ccc%10c(c9)c9ccccc9n%10-c9ccccc9)cc8c8ccccc8c7cc6c5c4)c4ccc5sc6ccccc6c5c4)ccc32)cc1 ; CC(C)(C)c1ccc(-c2cccc3c2oc2ccc(N(c4ccc5oc6ccccc6c5c4)c4ccc5sc6cc7c8ccc(N(c9ccc%10oc%11ccccc%11c%10c9)c9ccc%10oc%11c(-c%12ccc(C(C)(C)C)cc%12)cccc%11c%10c9)cc8c8ccccc8c7cc6c5c4)cc23)cc1]

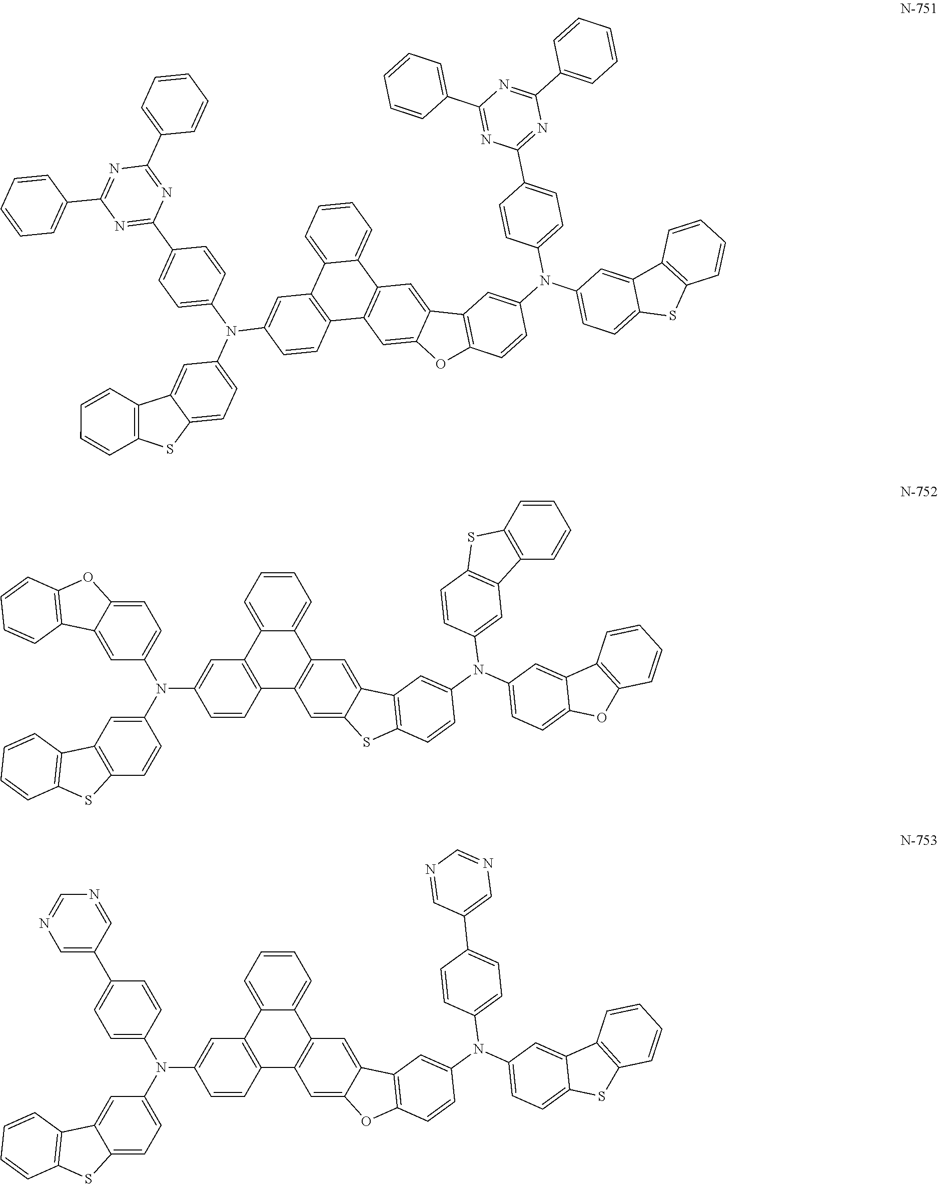 [SMILES: c1ccc(-c2nc(-c3ccccc3)nc(-c3ccc(N(c4ccc5oc6cc7c8ccc(N(c9ccc(-c%10nc(-c%11ccccc%11)nc(-c%11ccccc%11)n%10)cc9)c9ccc%10sc%11ccccc%11c%10c9)cc8c8ccccc8c7cc6c5c4)c4ccc5sc6ccccc6c5c4)cc3)n2)cc1 ; c1ccc2c(c1)oc1ccc(N(c3ccc4sc5ccccc5c4c3)c3ccc4sc5cc6c7ccc(N(c8ccc9oc%10ccccc%10c9c8)c8ccc9sc%10ccccc%10c9c8)cc7c7ccccc7c6cc5c4c3)cc12 ; c1ccc2c(c1)sc1ccc(N(c3ccc(-c4cncnc4)cc3)c3ccc4oc5cc6c7ccc(N(c8ccc(-c9cncnc9)cc8)c8ccc9sc%10ccccc%10c9c8)cc7c7ccccc7c6cc5c4c3)cc12]

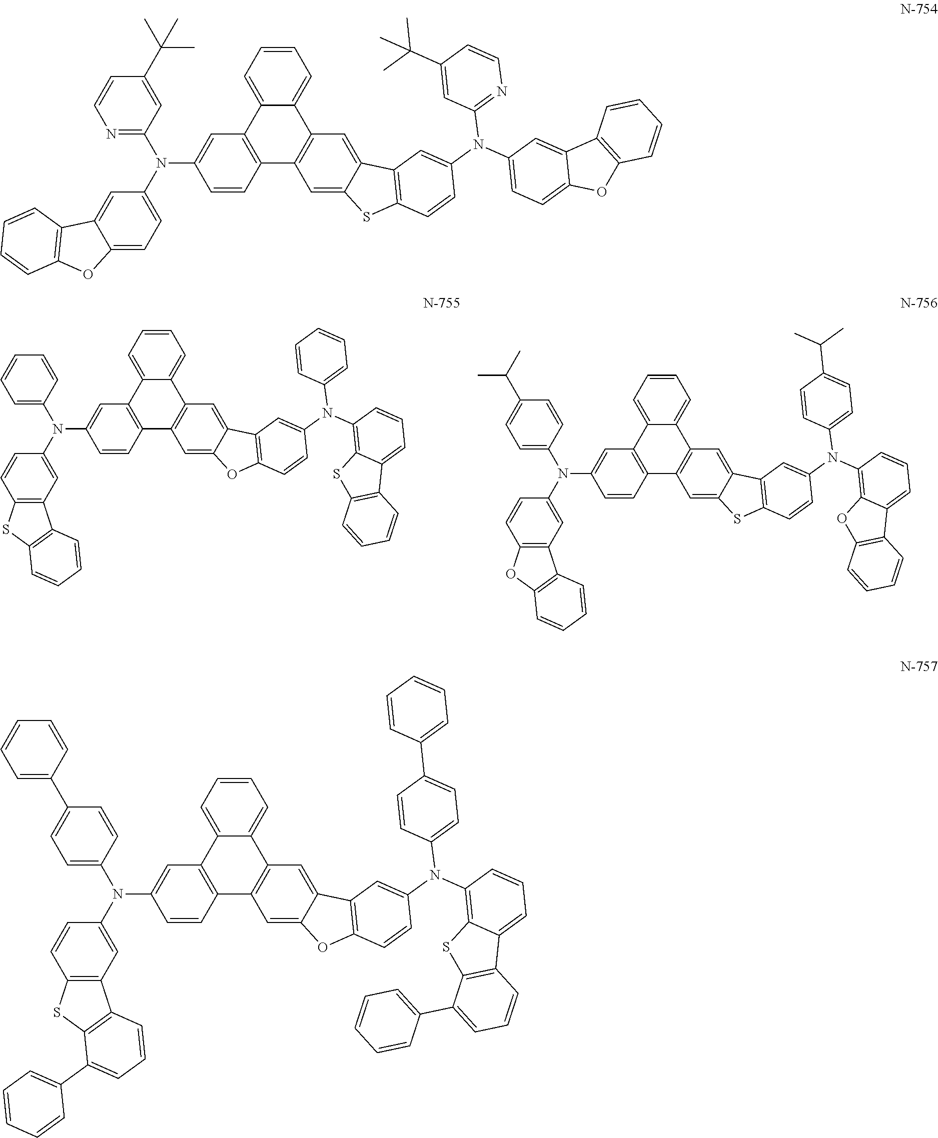 [SMILES: CC(C)(C)c1ccnc(N(c2ccc3oc4ccccc4c3c2)c2ccc3sc4cc5c6ccc(N(c7ccc8oc9ccccc9c8c7)c7cc(C(C)(C)C)ccn7)cc6c6ccccc6c5cc4c3c2)c1 ; c1ccc(N(c2ccc3sc4ccccc4c3c2)c2ccc3c(c2)c2ccccc2c2cc4c(cc32)oc2ccc(N(c3ccccc3)c3cccc5c3sc3ccccc35)cc24)cc1 ; CC(C)c1ccc(N(c2ccc3oc4ccccc4c3c2)c2ccc3c(c2)c2ccccc2c2cc4c(cc32)sc2ccc(N(c3ccc(C(C)C)cc3)c3cccc5c3oc3ccccc35)cc24)cc1 ; c1ccc(-c2ccc(N(c3ccc4sc5c(-c6ccccc6)cccc5c4c3)c3ccc4c(c3)c3ccccc3c3cc5c(cc43)oc3ccc(N(c4ccc(-c6ccccc6)cc4)c4cccc6c4sc4c(-c7ccccc7)cccc46)cc35)cc2)cc1]

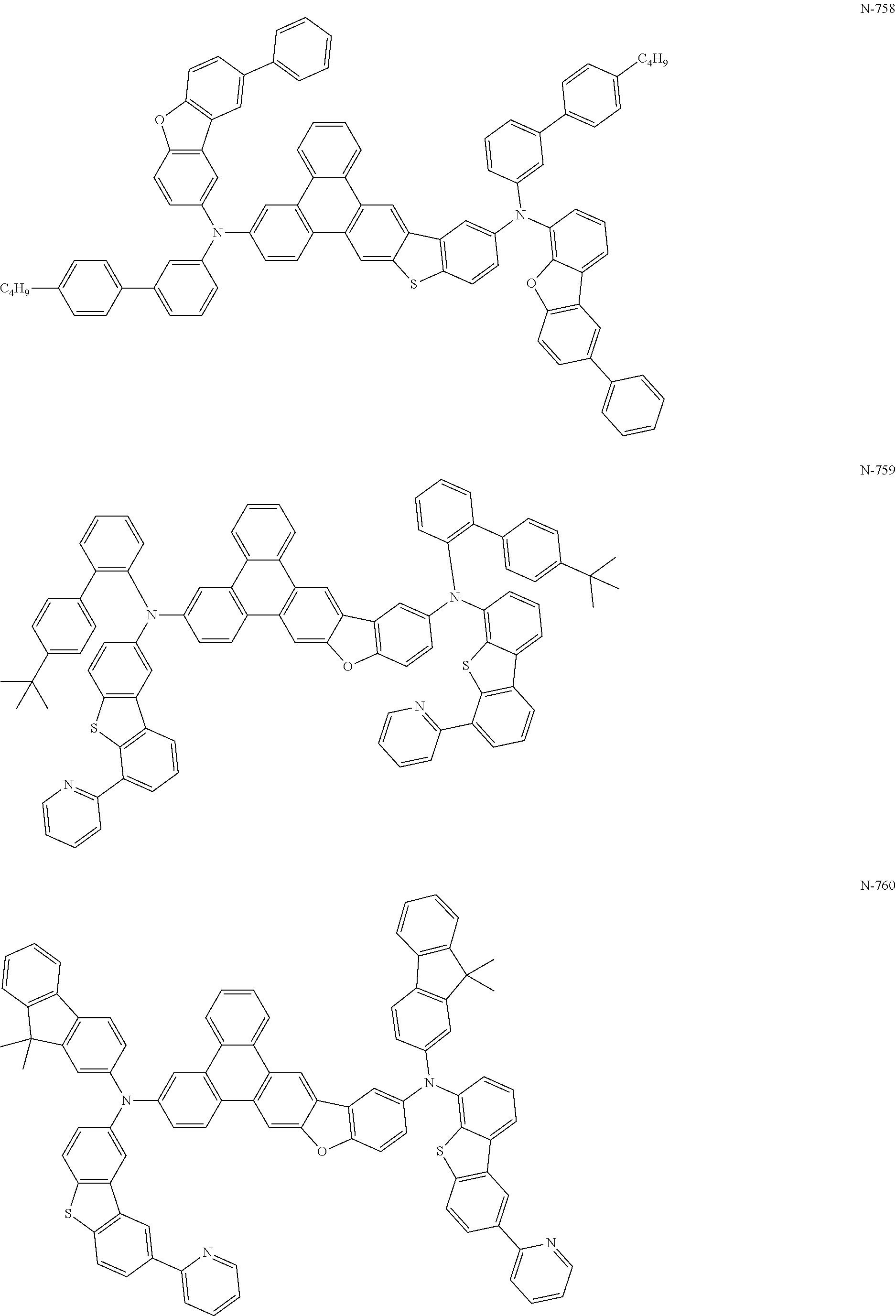

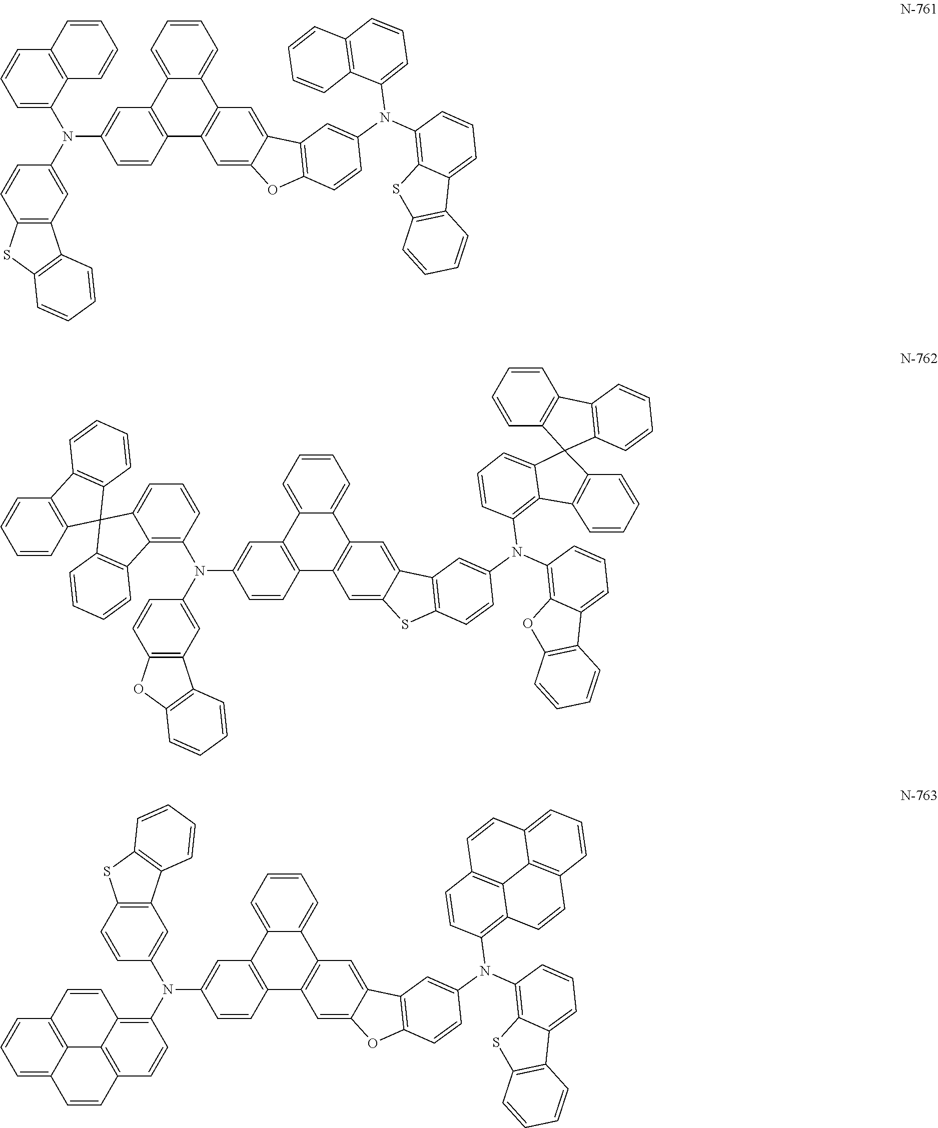 [SMILES: c1ccc2c(N(c3ccc4sc5ccccc5c4c3)c3ccc4c(c3)c3ccccc3c3cc5c(cc43)oc3ccc(N(c4cccc6ccccc46)c4cccc6c4sc4ccccc46)cc35)cccc2c1 ; c1ccc2c(c1)-c1ccccc1C21c2ccccc2-c2c(N(c3ccc4oc5ccccc5c4c3)c3ccc4c(c3)c3ccccc3c3cc5c(cc43)sc3ccc(N(c4cccc6c4-c4ccccc4C64c6ccccc6-c6ccccc64)c4cccc6c4oc4ccccc46)cc35)cccc21 ; c1cc2ccc3ccc(N(c4ccc5sc6ccccc6c5c4)c4ccc5c(c4)c4ccccc4c4cc6c(cc54)oc4ccc(N(c5ccc7ccc8cccc9ccc5c7c89)c5cccc7c5sc5ccccc57)cc46)c4ccc(c1)c2c34]

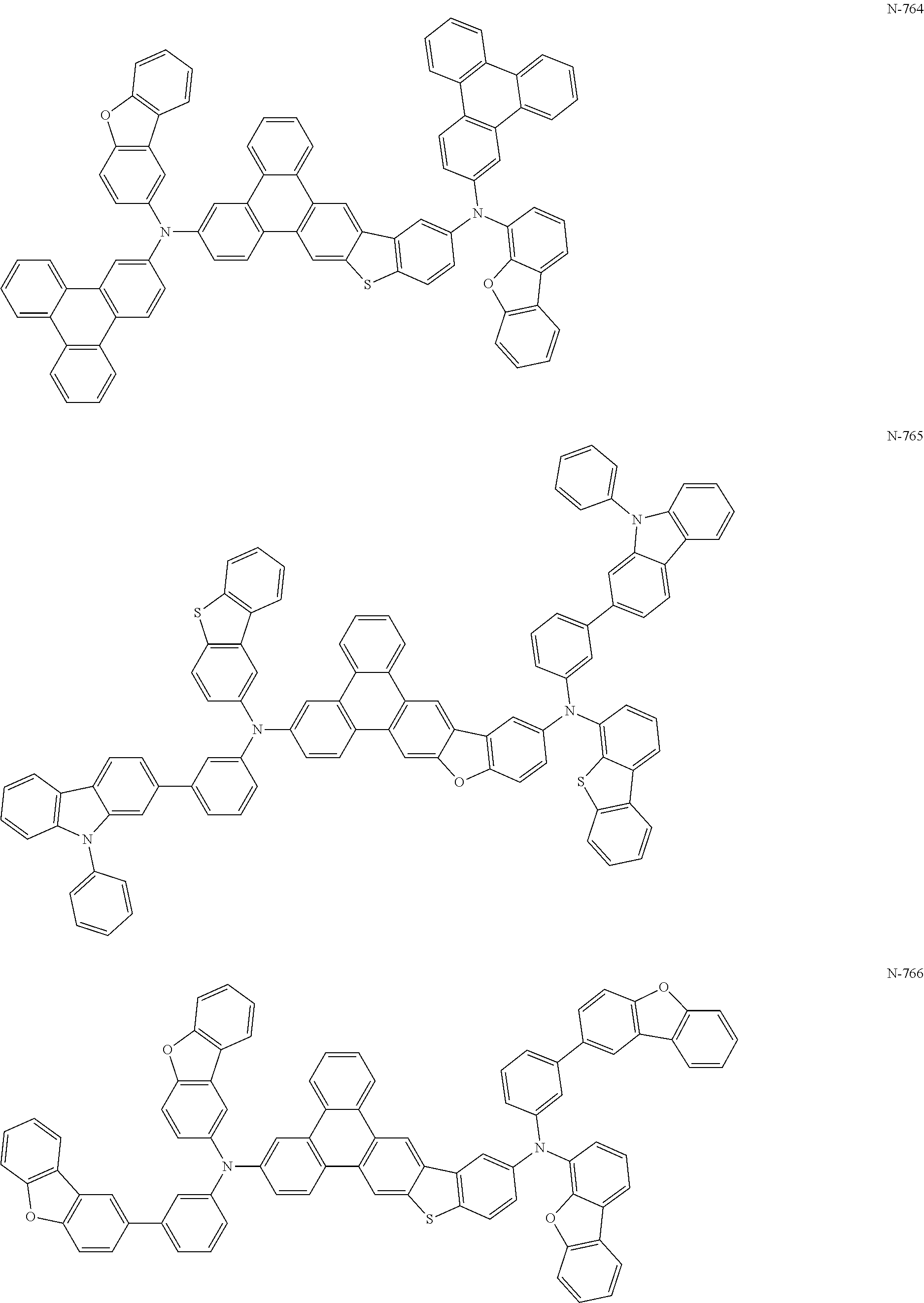 [SMILES: c1ccc2c(c1)oc1ccc(N(c3ccc4c5ccccc5c5ccccc5c4c3)c3ccc4c(c3)c3ccccc3c3cc5c(cc43)sc3ccc(N(c4ccc6c7ccccc7c7ccccc7c6c4)c4cccc6c4oc4ccccc46)cc35)cc12 ; c1ccc(-n2c3ccccc3c3ccc(-c4cccc(N(c5ccc6sc7ccccc7c6c5)c5ccc6c(c5)c5ccccc5c5cc7c(cc65)oc5ccc(N(c6cccc(-c8ccc9c%10ccccc%10n(-c%10ccccc%10)c9c8)c6)c6cccc8c6sc6ccccc68)cc57)c4)cc32)cc1 ; c1cc(-c2ccc3oc4ccccc4c3c2)cc(N(c2ccc3oc4ccccc4c3c2)c2ccc3c(c2)c2ccccc2c2cc4c(cc32)sc2ccc(N(c3cccc(-c5ccc6oc7ccccc7c6c5)c3)c3cccc5c3oc3ccccc35)cc24)c1]

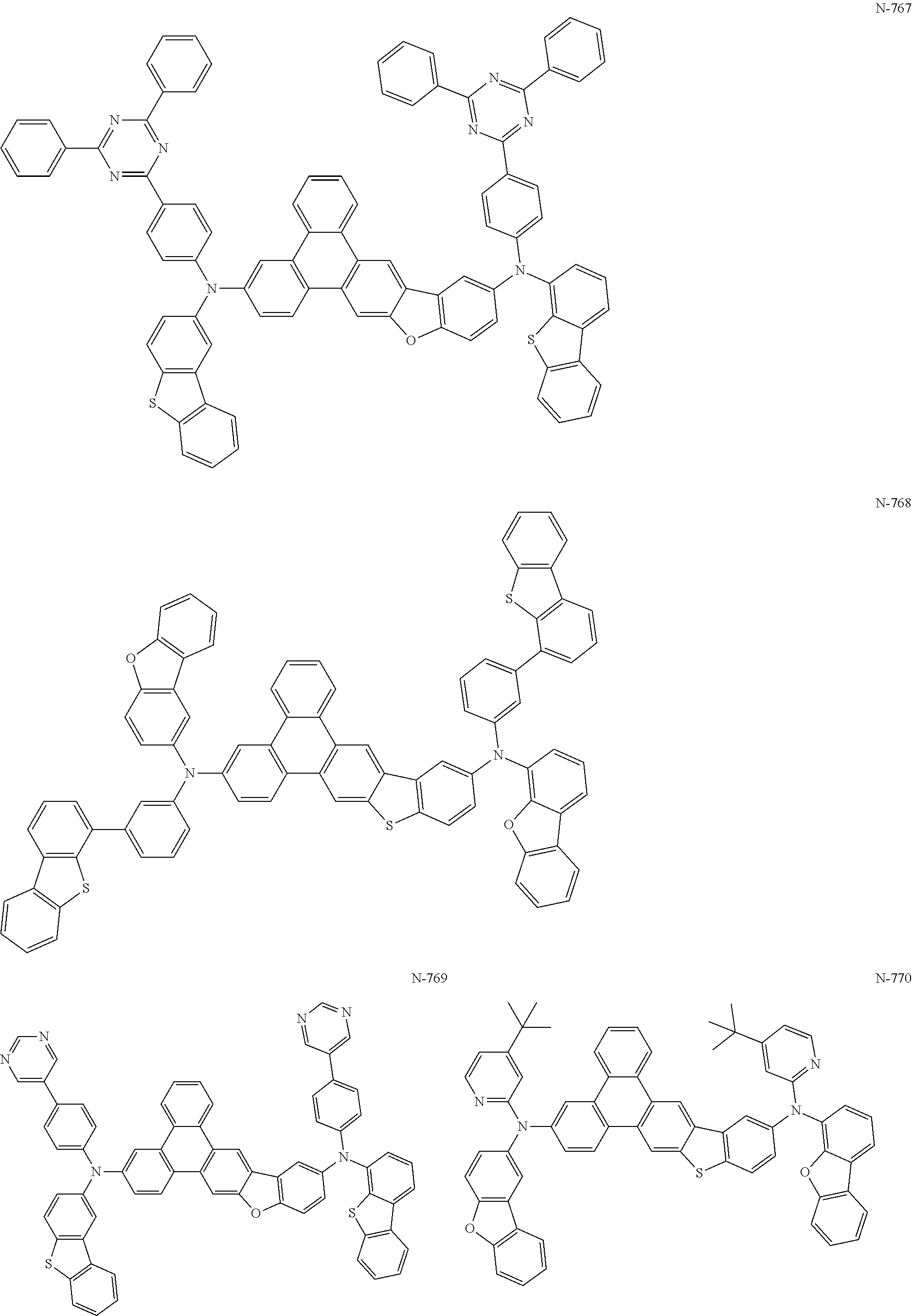

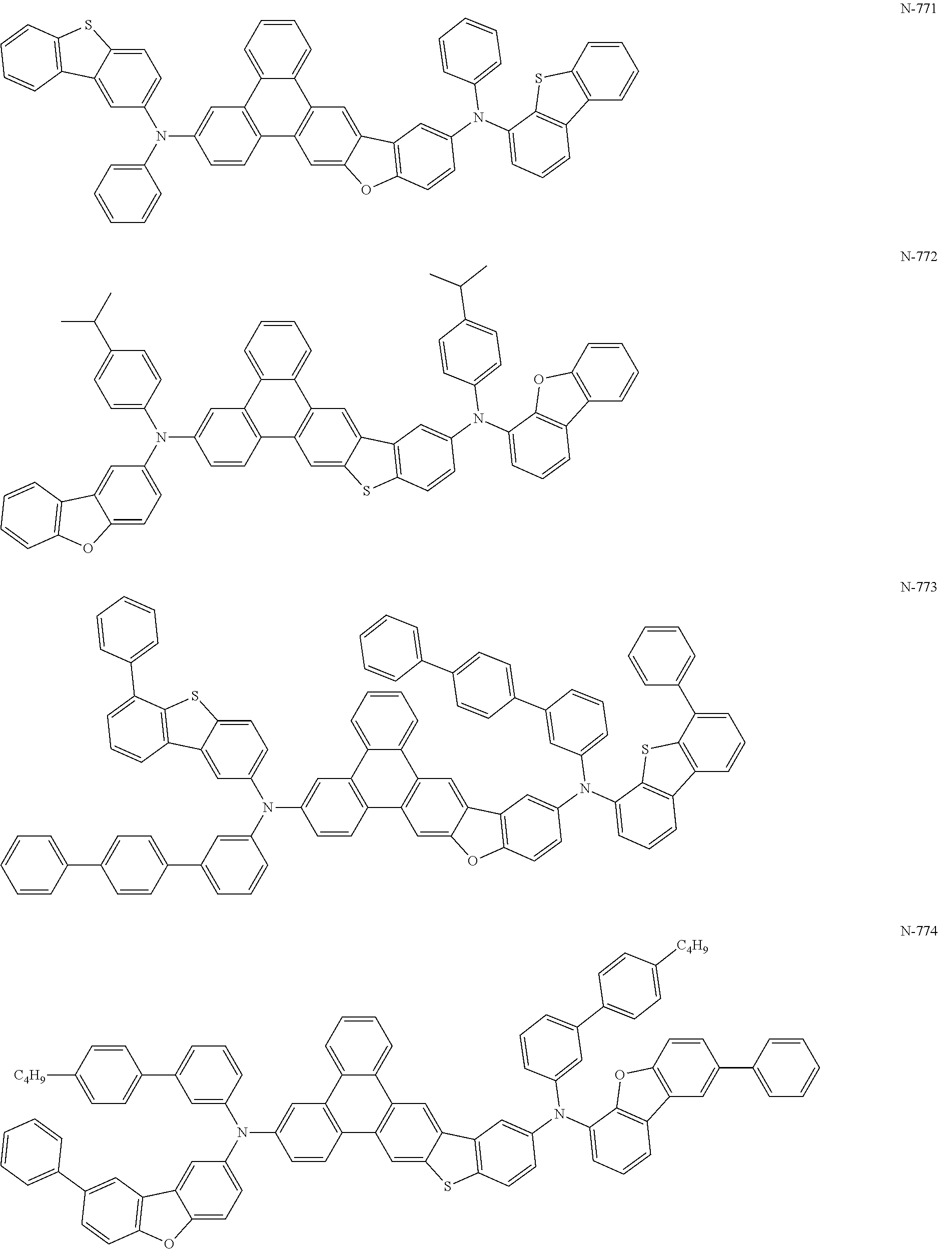

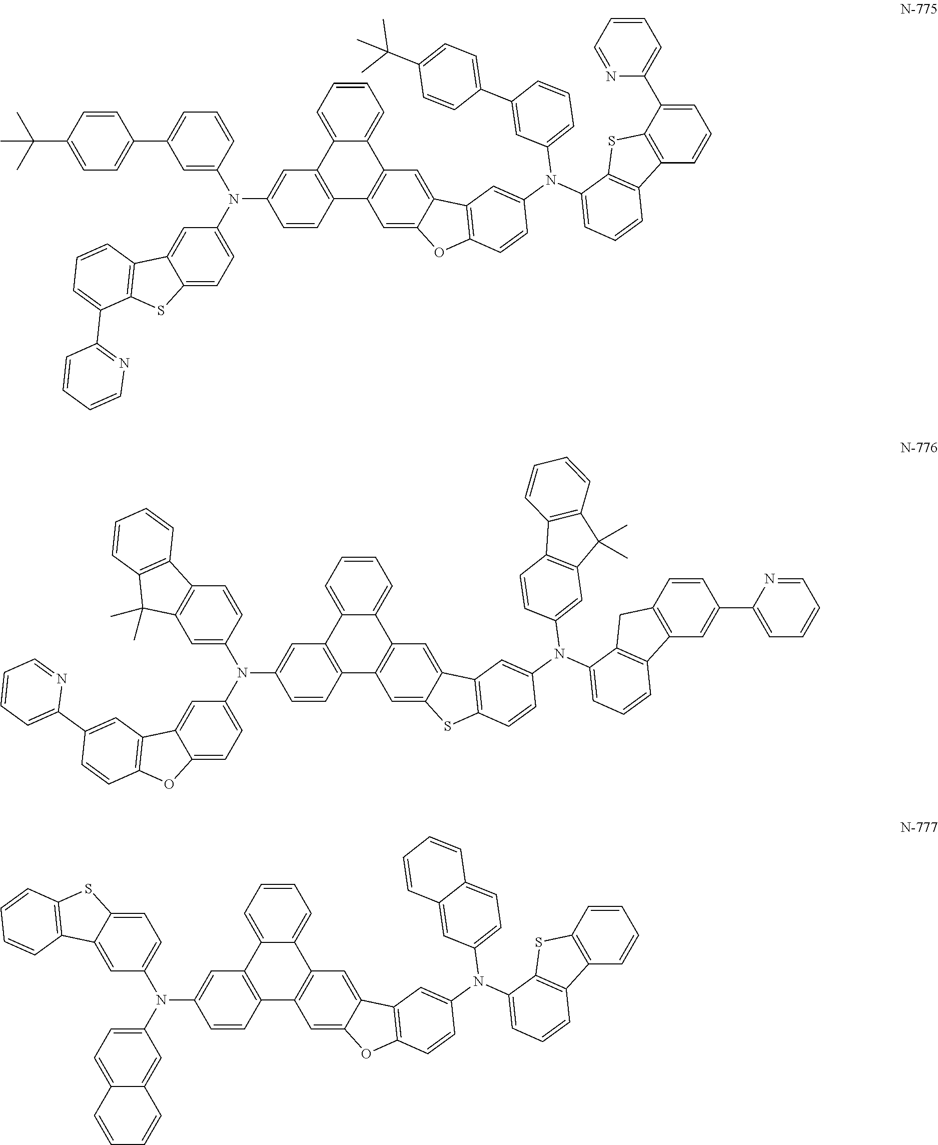 [SMILES: CC(C)(C)c1ccc(-c2cccc(N(c3ccc4sc5c(-c6ccccn6)cccc5c4c3)c3ccc4c(c3)c3ccccc3c3cc5c(cc43)oc3ccc(N(c4cccc(-c6ccc(C(C)(C)C)cc6)c4)c4cccc6c4sc4c(-c7ccccn7)cccc46)cc35)c2)cc1 ; CC1(C)c2ccccc2-c2ccc(N(c3ccc4oc5ccc(-c6ccccn6)cc5c4c3)c3ccc4c(c3)c3ccccc3c3cc5c(cc43)sc3ccc(N(c4ccc6c(c4)C(C)(C)c4ccccc4-6)c4cccc6c4Cc4ccc(-c7ccccn7)cc4-6)cc35)cc21 ; c1ccc2cc(N(c3ccc4sc5ccccc5c4c3)c3ccc4c(c3)c3ccccc3c3cc5c(cc43)oc3ccc(N(c4ccc6ccccc6c4)c4cccc6c4sc4ccccc46)cc35)ccc2c1]

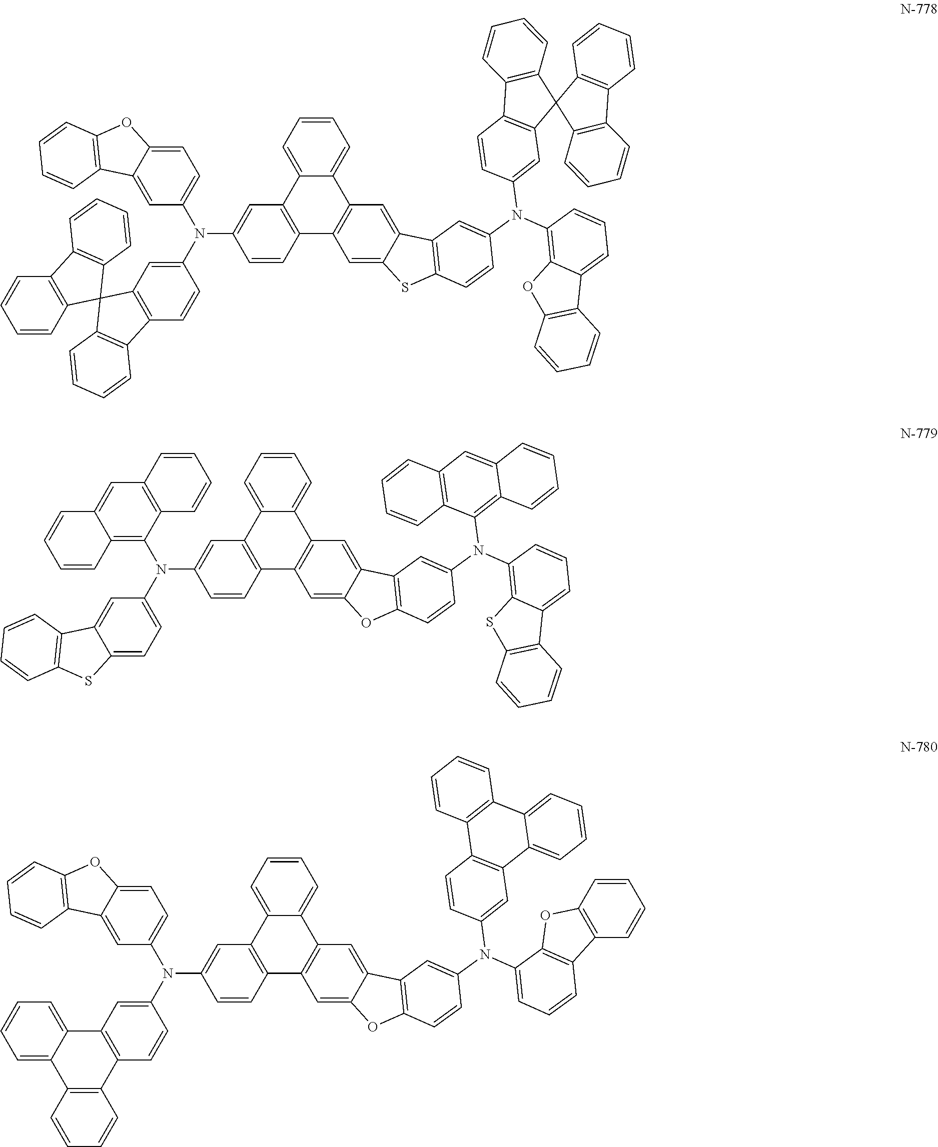 [SMILES: c1ccc2c(c1)-c1ccccc1C21c2ccccc2-c2ccc(N(c3ccc4oc5ccccc5c4c3)c3ccc4c(c3)c3ccccc3c3cc5c(cc43)sc3ccc(N(c4ccc6c(c4)C4(c7ccccc7-c7ccccc74)c4ccccc4-6)c4cccc6c4oc4ccccc46)cc35)cc21 ; c1ccc2c(N(c3ccc4sc5ccccc5c4c3)c3ccc4c(c3)c3ccccc3c3cc5c(cc43)oc3ccc(N(c4c6ccccc6cc6ccccc46)c4cccc6c4sc4ccccc46)cc35)c3ccccc3cc2c1 ; c1ccc2c(c1)oc1ccc(N(c3ccc4c5ccccc5c5ccccc5c4c3)c3ccc4c(c3)c3ccccc3c3cc5c(cc43)oc3ccc(N(c4ccc6c7ccccc7c7ccccc7c6c4)c4cccc6c4oc4ccccc46)cc35)cc12]

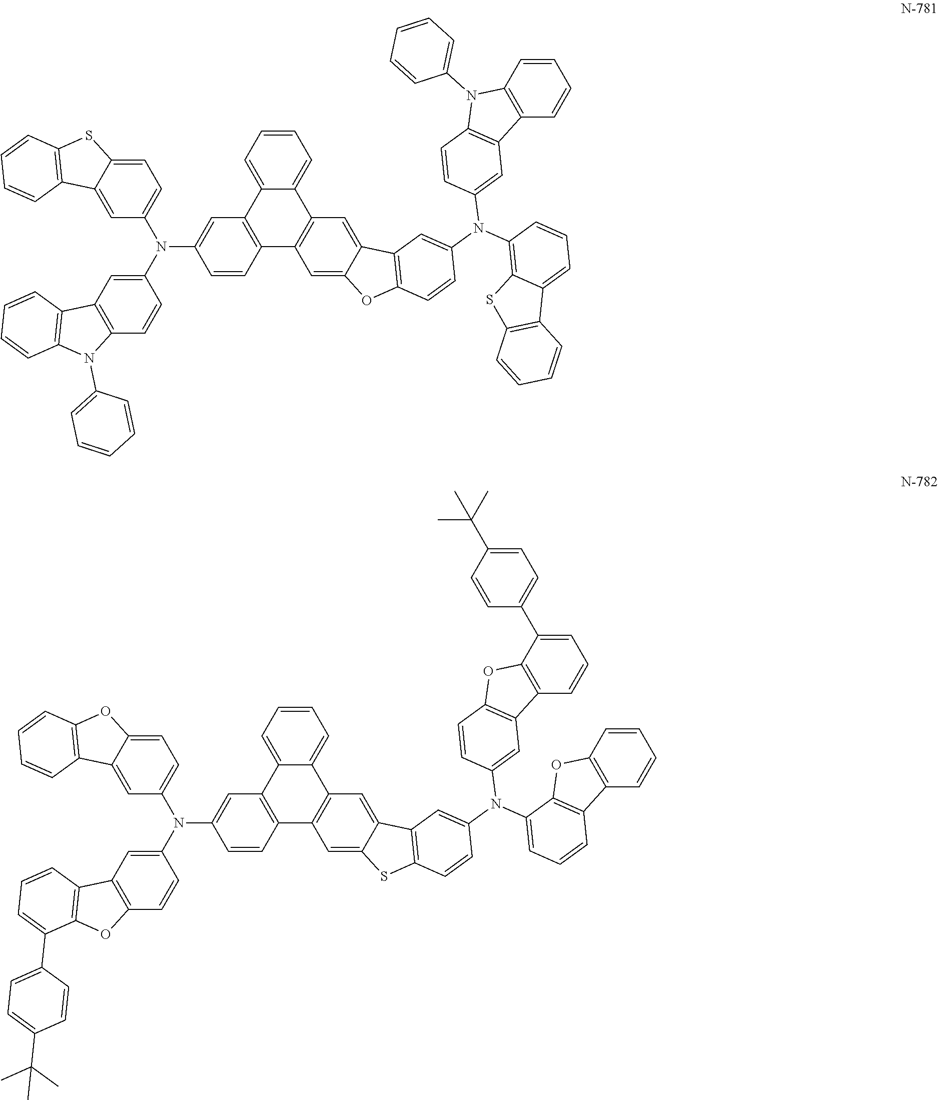 [SMILES: c1ccc(-n2c3ccccc3c3cc(N(c4ccc5sc6ccccc6c5c4)c4ccc5c(c4)c4ccccc4c4cc6c(cc54)oc4ccc(N(c5ccc7c(c5)c5ccccc5n7-c5ccccc5)c5cccc7c5sc5ccccc57)cc46)ccc32)cc1 ; CC(C)(C)c1ccc(-c2cccc3c2oc2ccc(N(c4ccc5oc6ccccc6c5c4)c4ccc5c(c4)c4ccccc4c4cc6c(cc54)sc4ccc(N(c5ccc7oc8c(-c9ccc(C(C)(C)C)cc9)cccc8c7c5)c5cccc7c5oc5ccccc57)cc46)cc23)cc1]

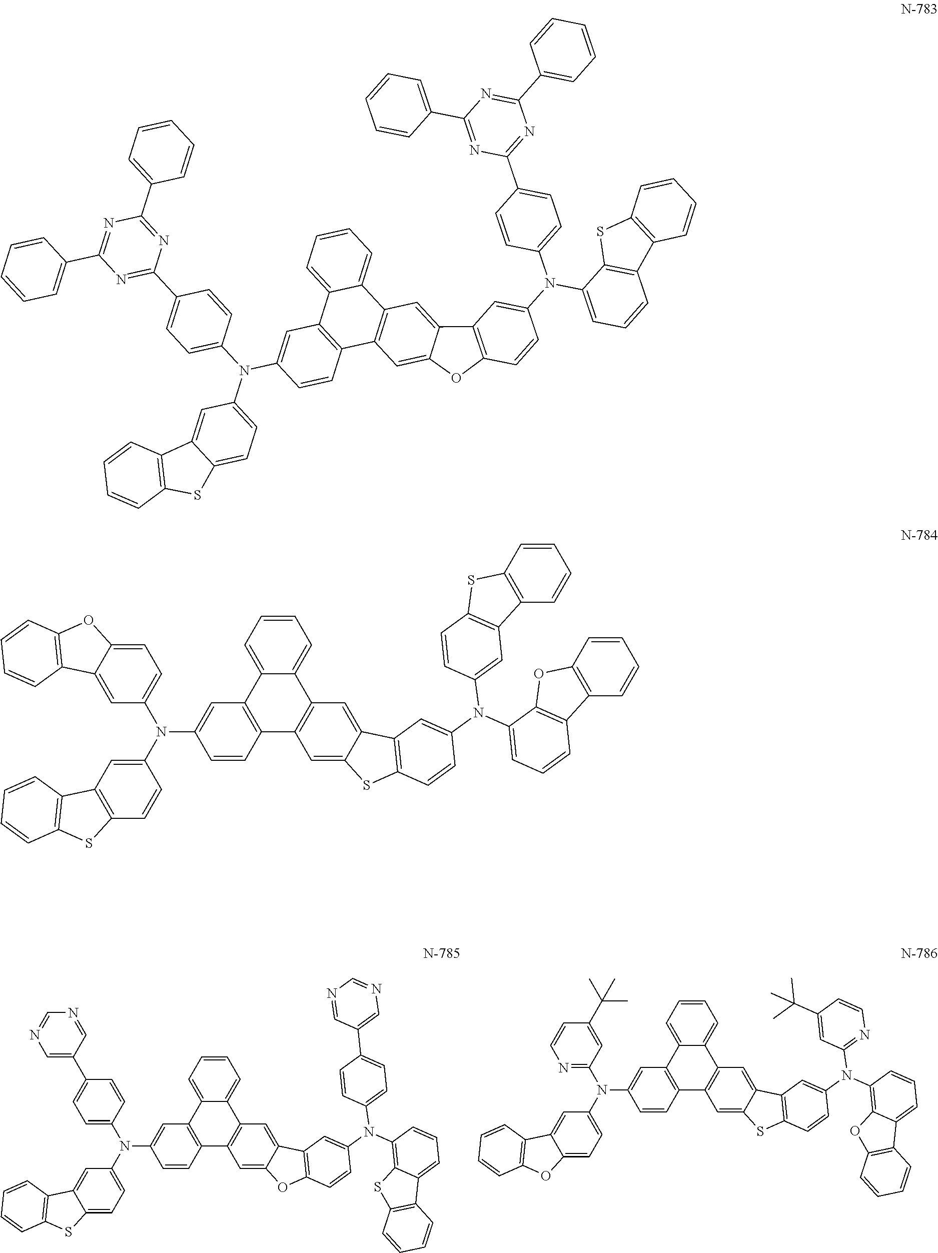

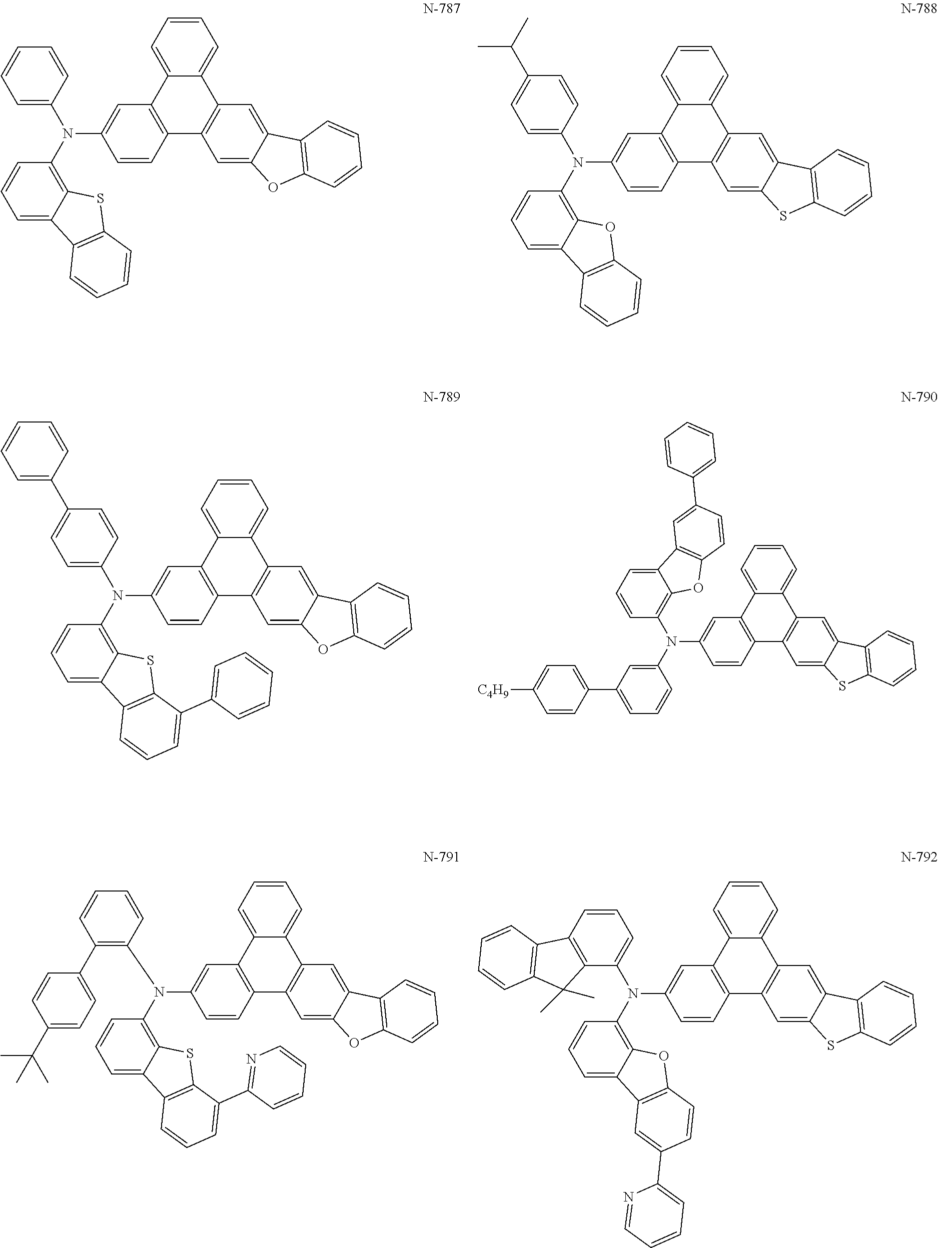

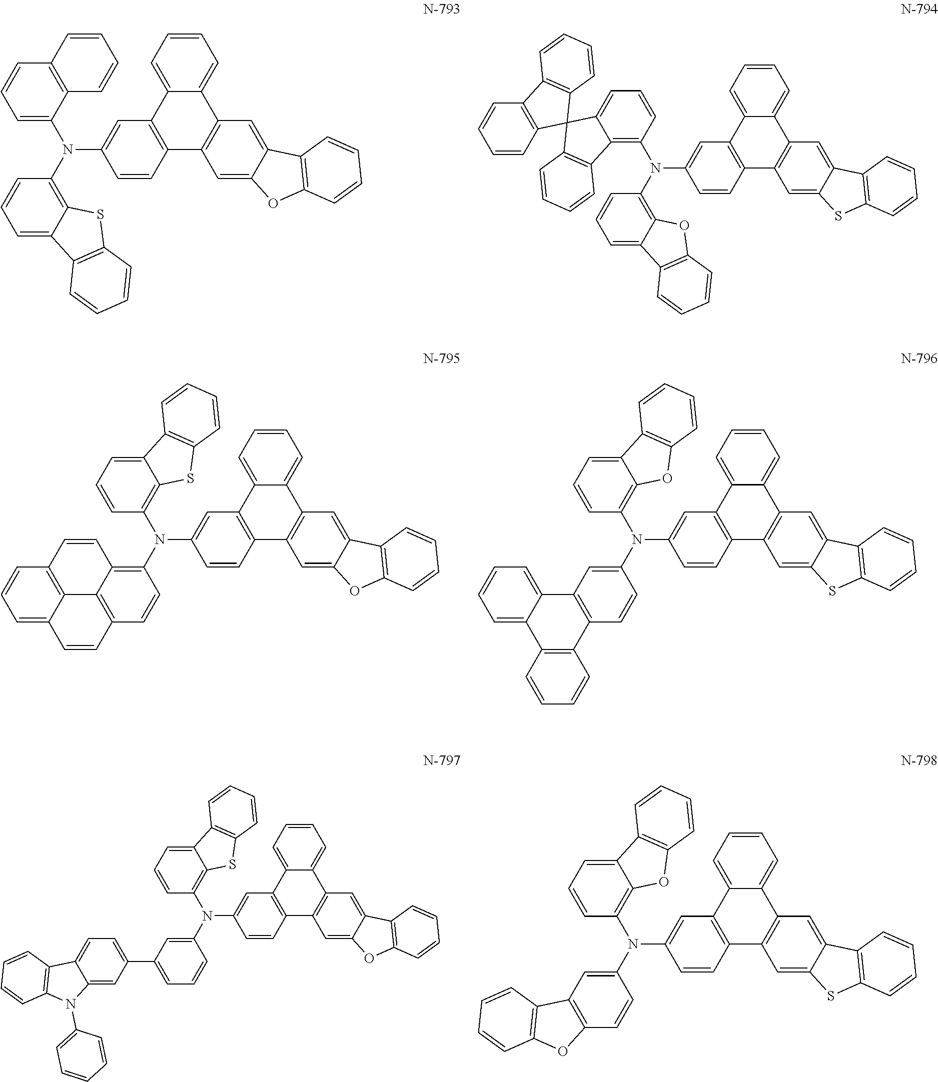

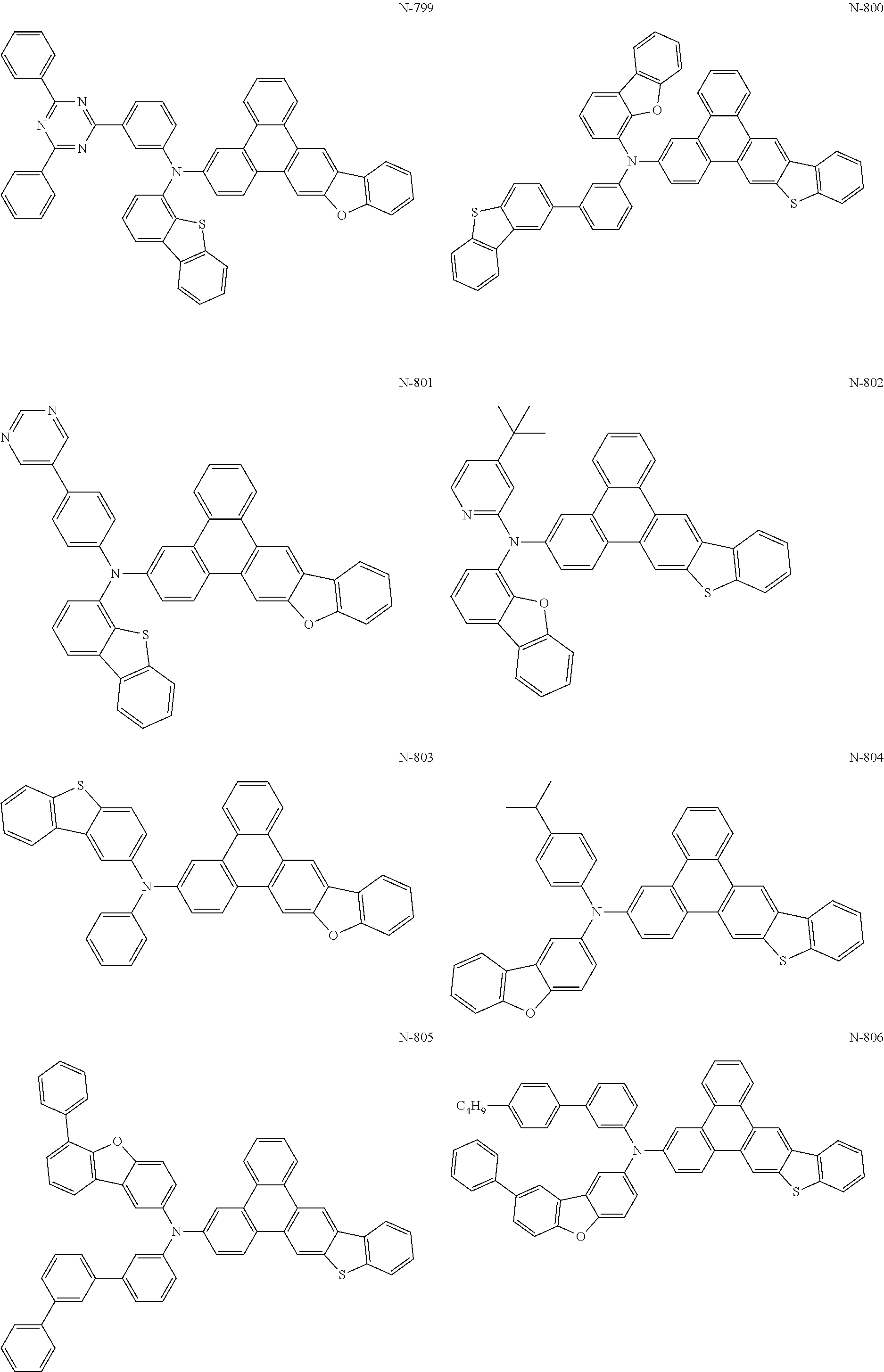

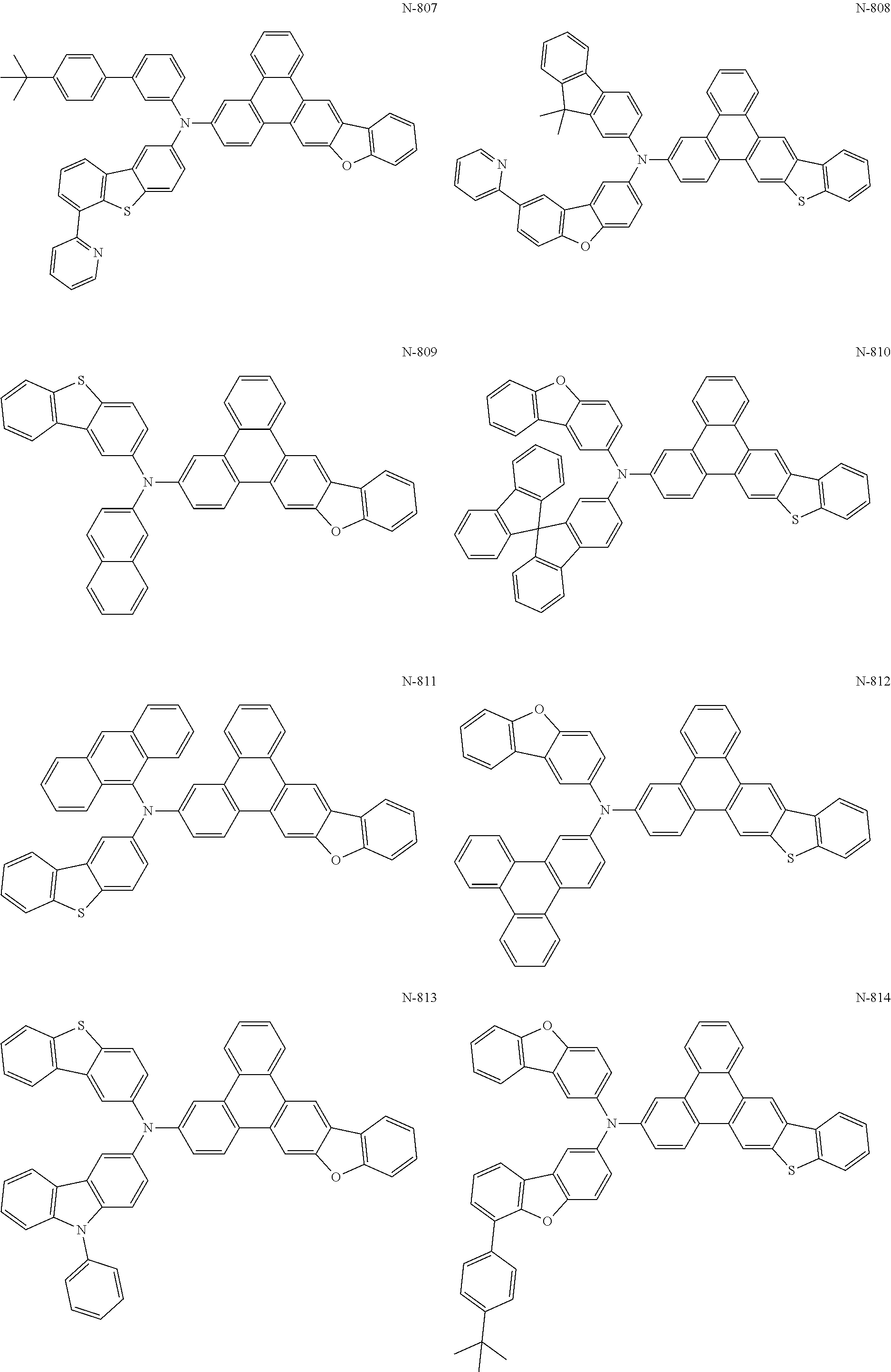

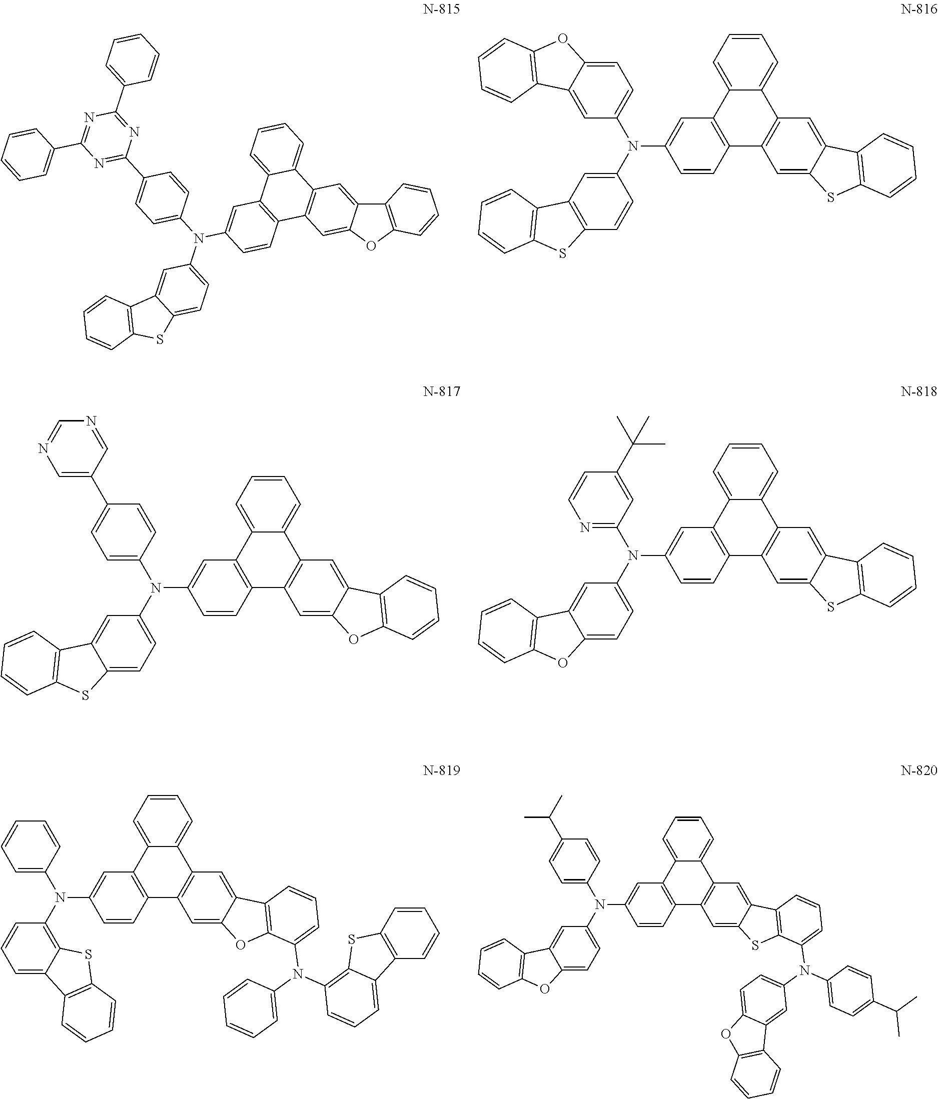

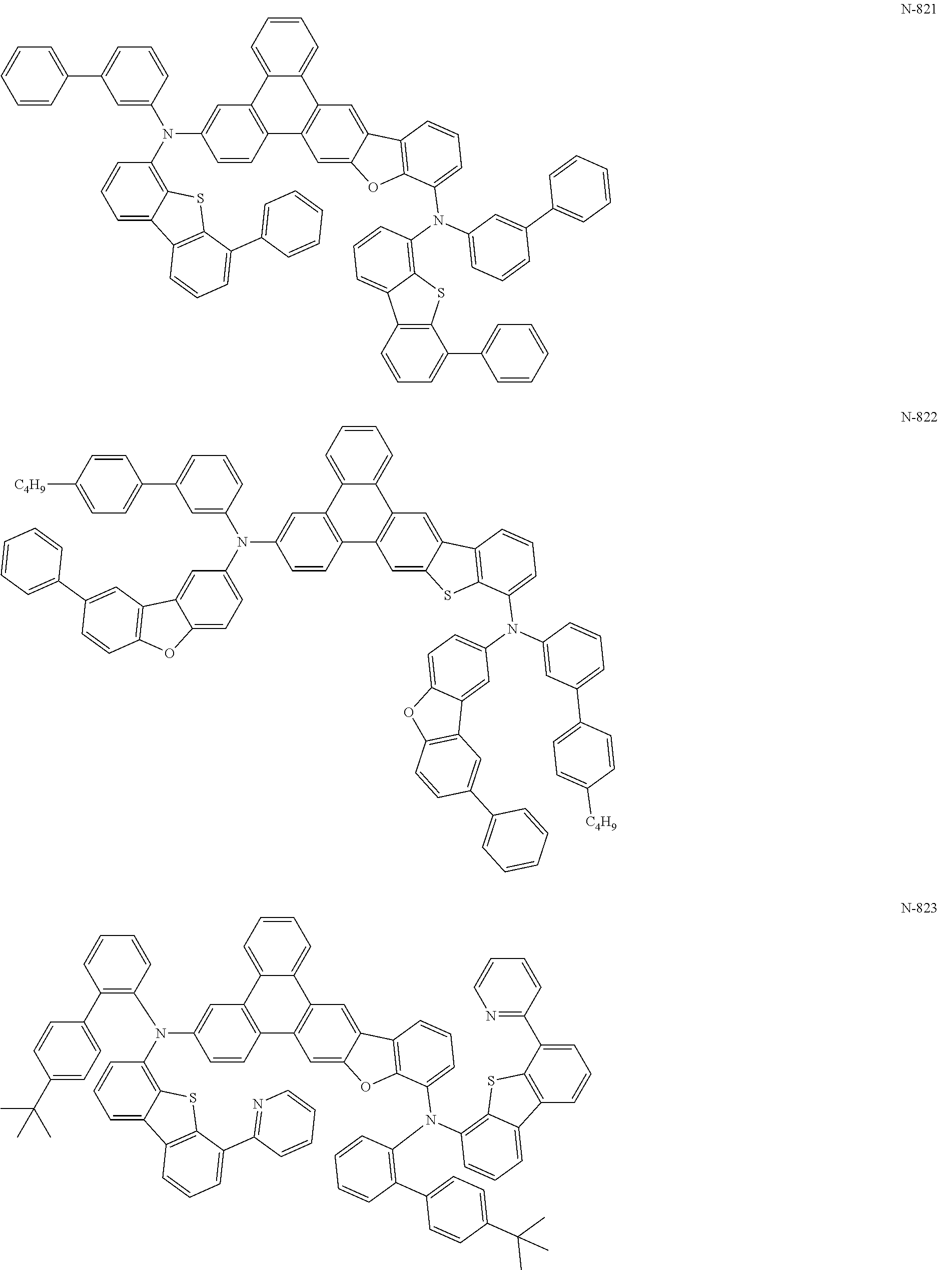

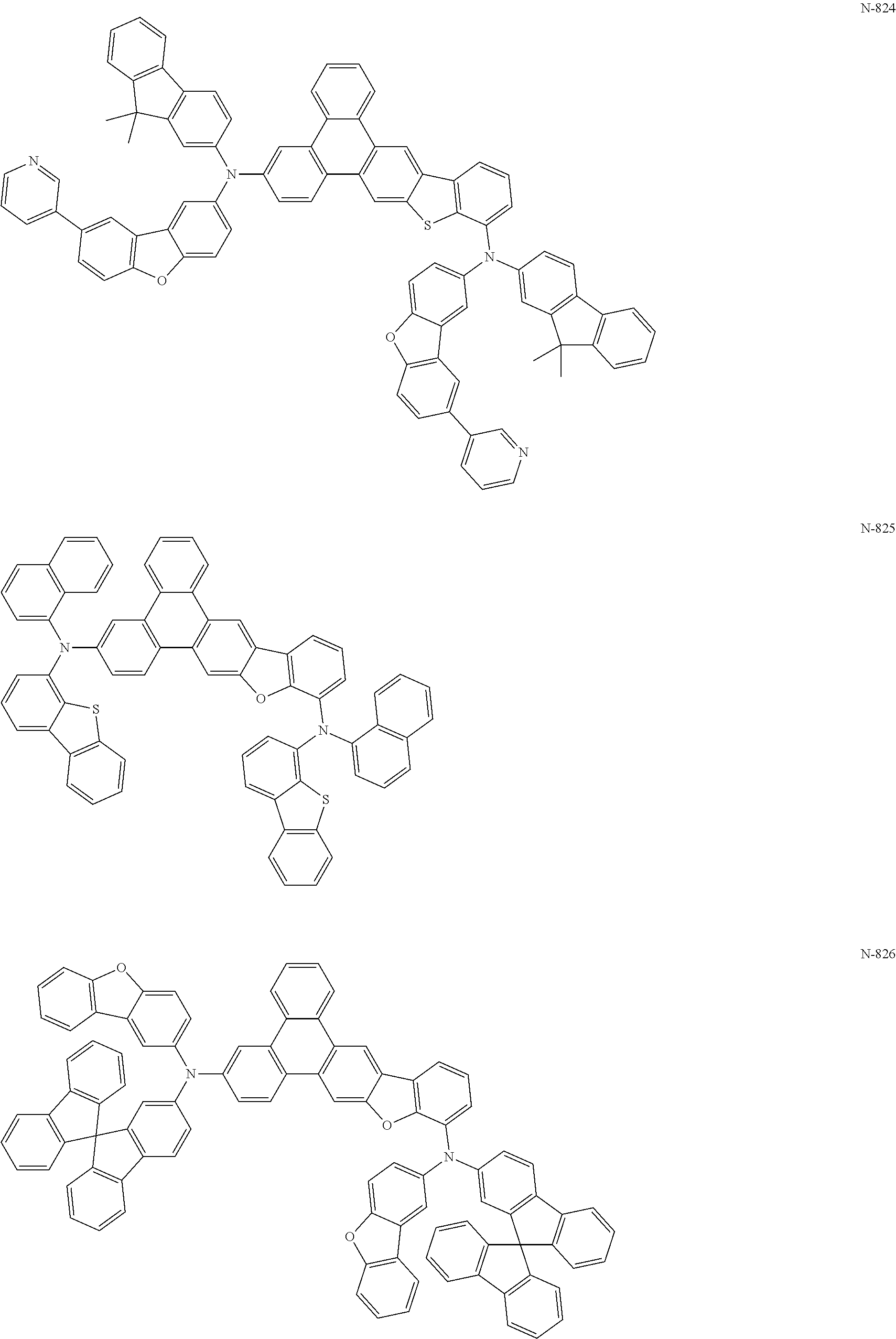 [SMILES: CC1(C)c2ccccc2-c2ccc(N(c3ccc4oc5ccc(-c6cccnc6)cc5c4c3)c3ccc4c(c3)c3ccccc3c3cc5c(cc43)sc3c(N(c4ccc6c(c4)C(C)(C)c4ccccc4-6)c4ccc6oc7ccc(-c8cccnc8)cc7c6c4)cccc35)cc21 ; c1ccc2c(N(c3ccc4c(c3)c3ccccc3c3cc5c(cc43)oc3c(N(c4cccc6ccccc46)c4cccc6c4sc4ccccc46)cccc35)c3cccc4c3sc3ccccc34)cccc2c1 ; c1ccc2c(c1)-c1ccccc1C21c2ccccc2-c2ccc(N(c3ccc4oc5ccccc5c4c3)c3ccc4c(c3)c3ccccc3c3cc5c(cc43)oc3c(N(c4ccc6c(c4)C4(c7ccccc7-c7ccccc74)c4ccccc4-6)c4ccc6oc7ccccc7c6c4)cccc35)cc21]

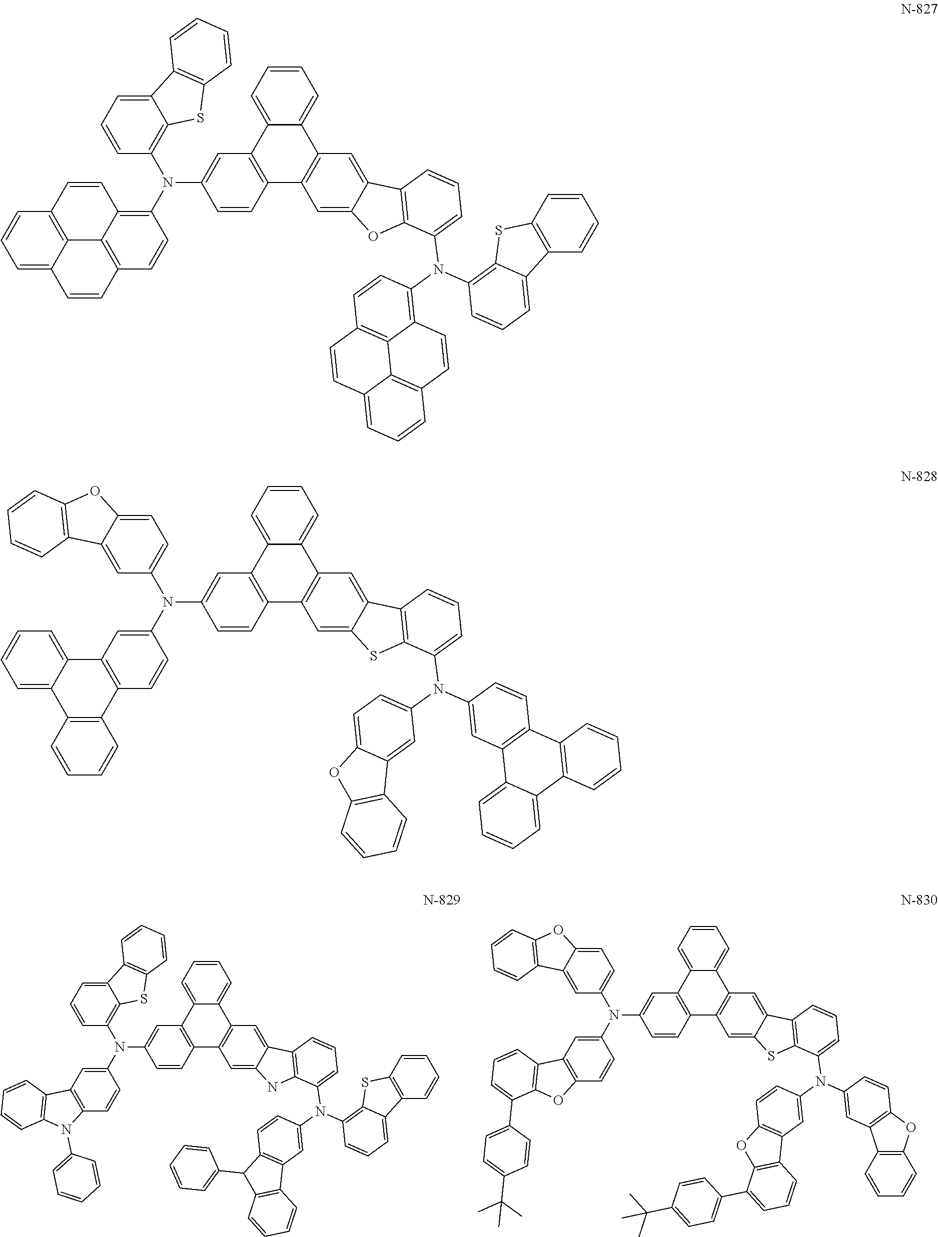

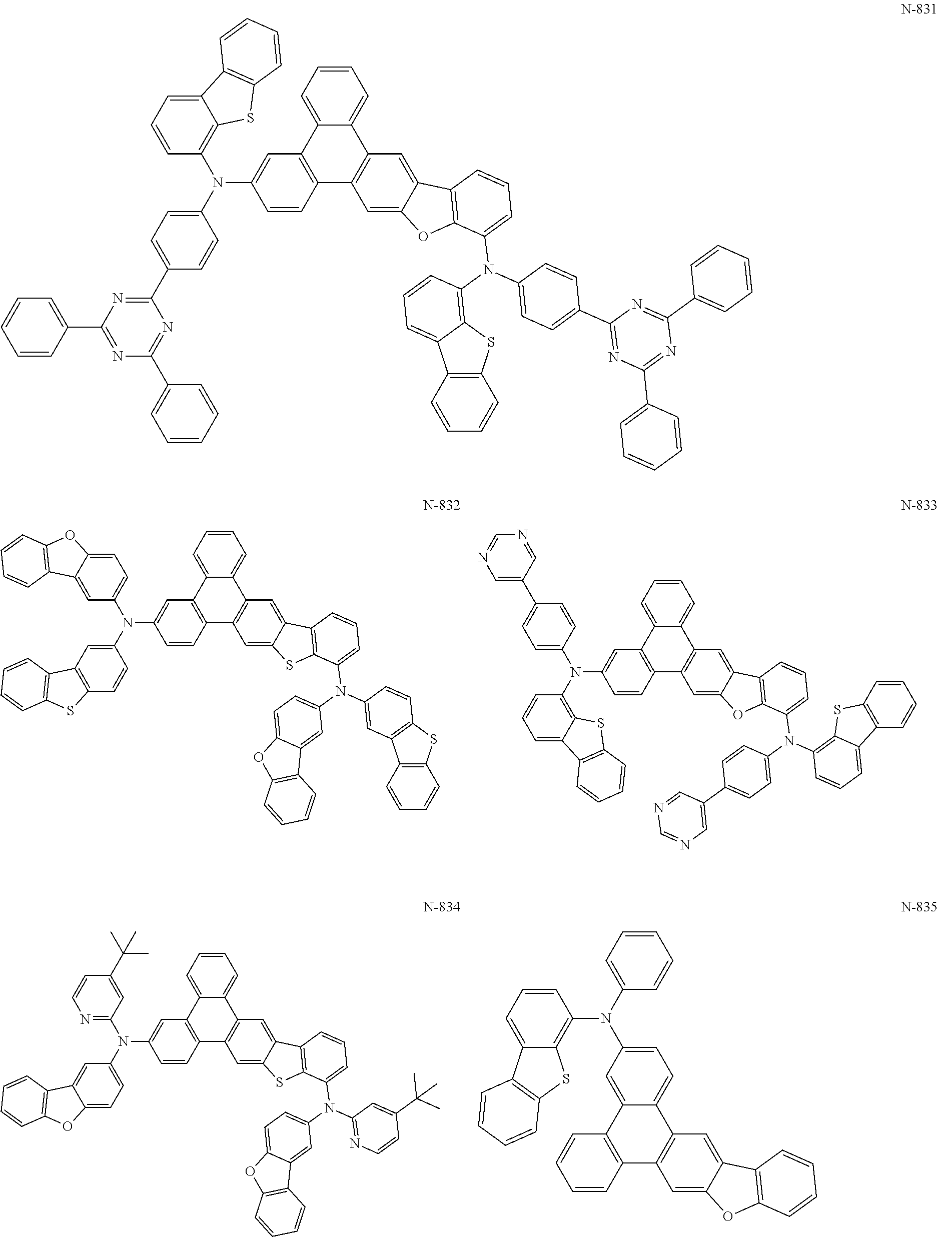

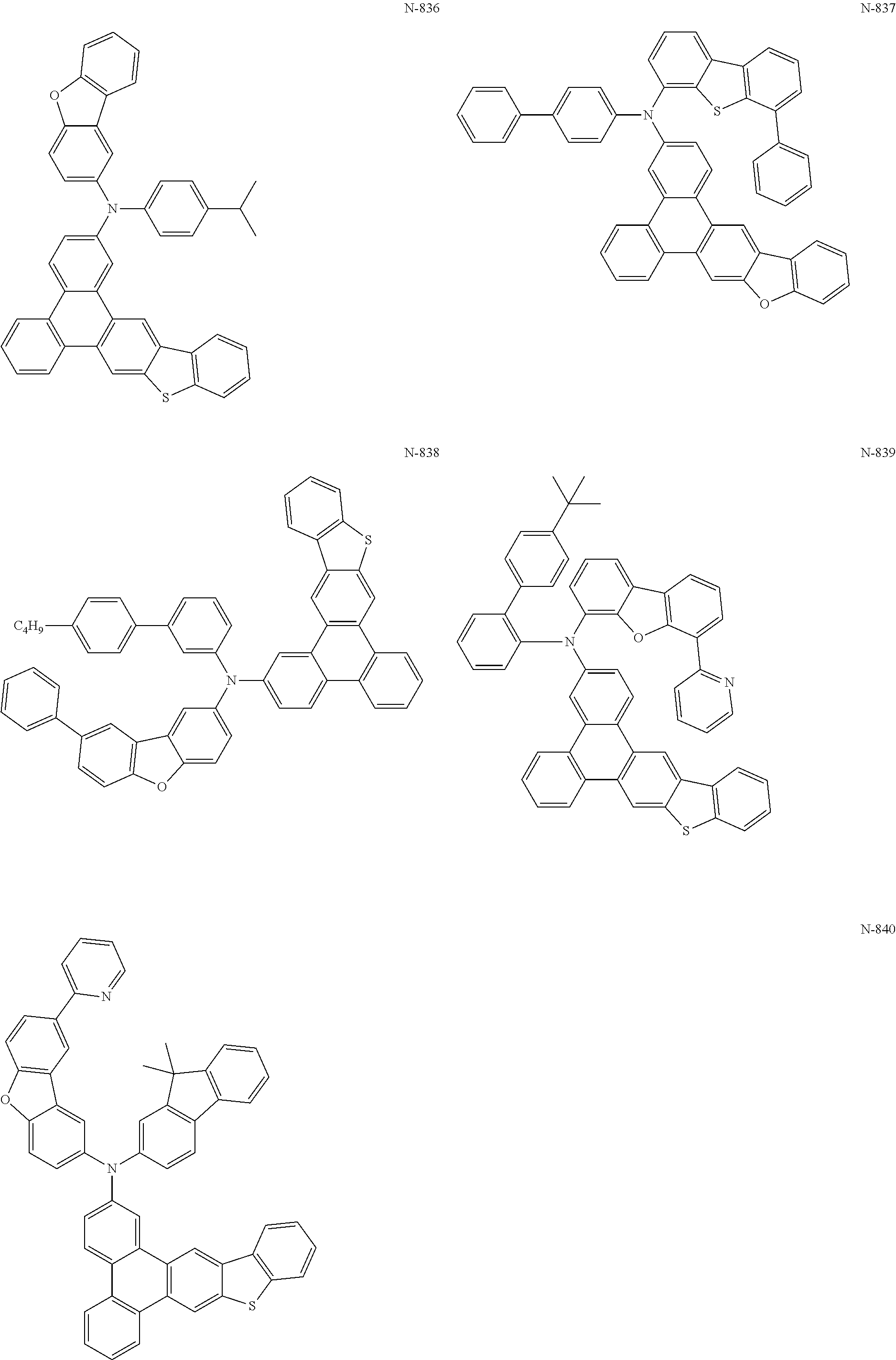

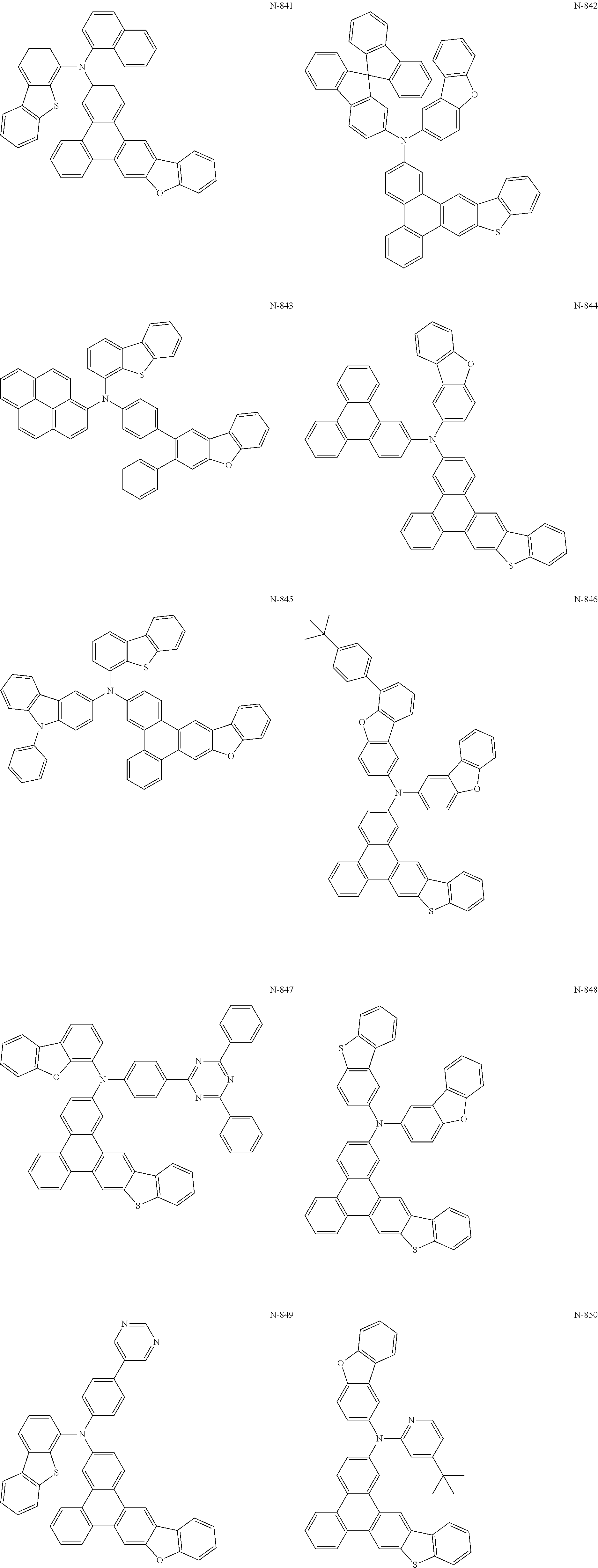

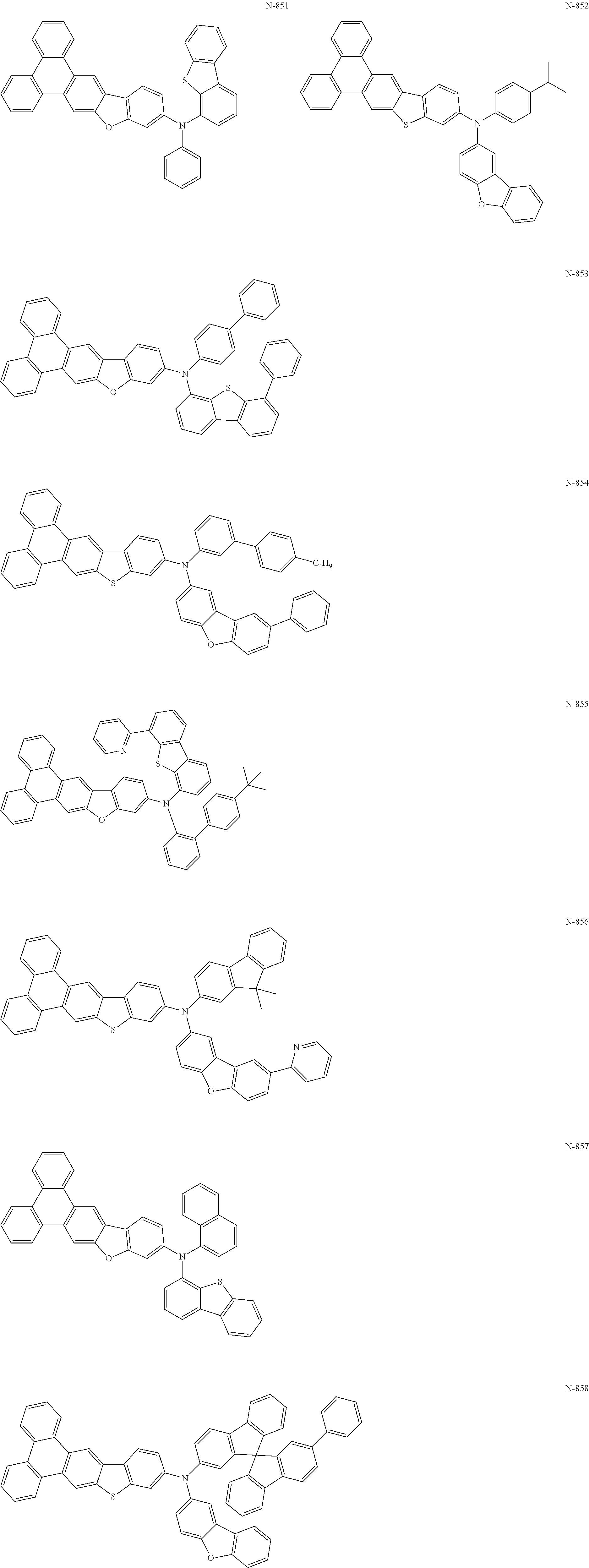

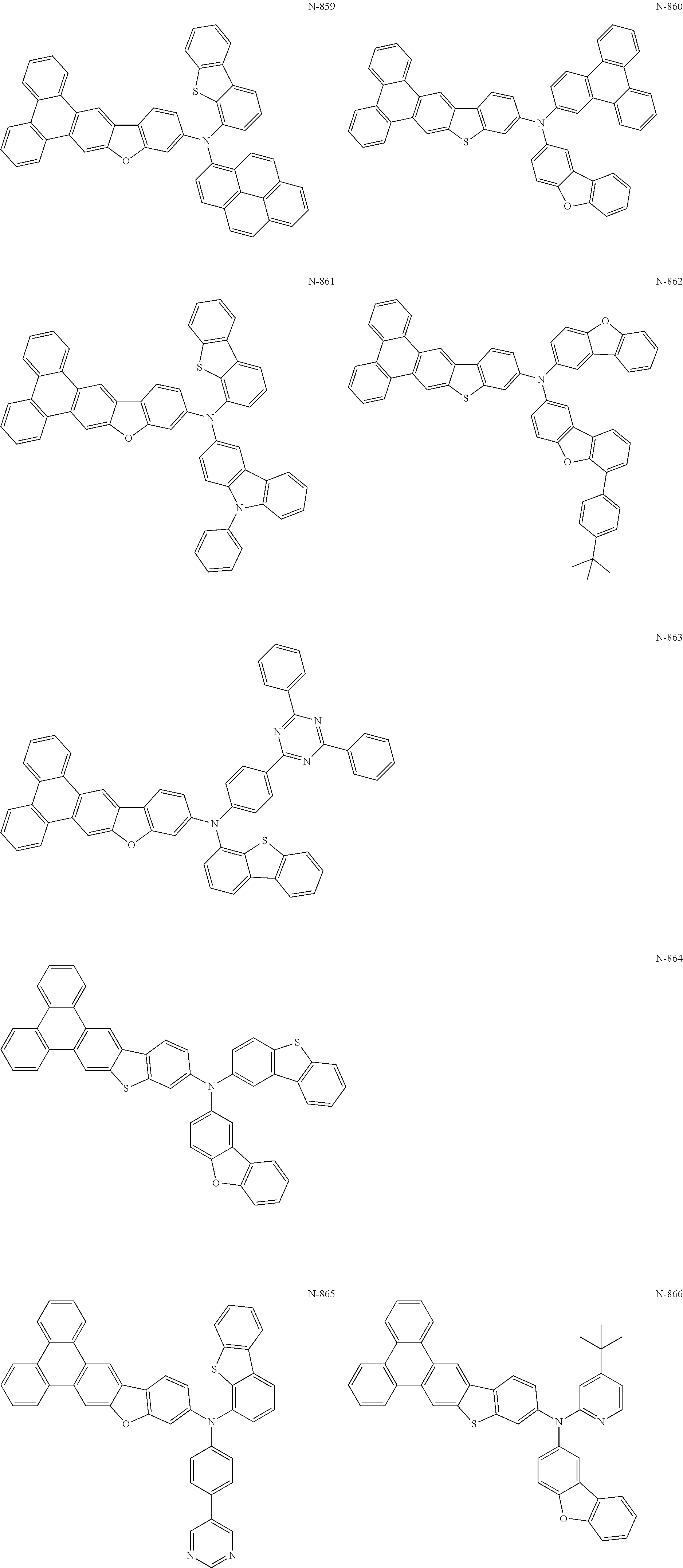

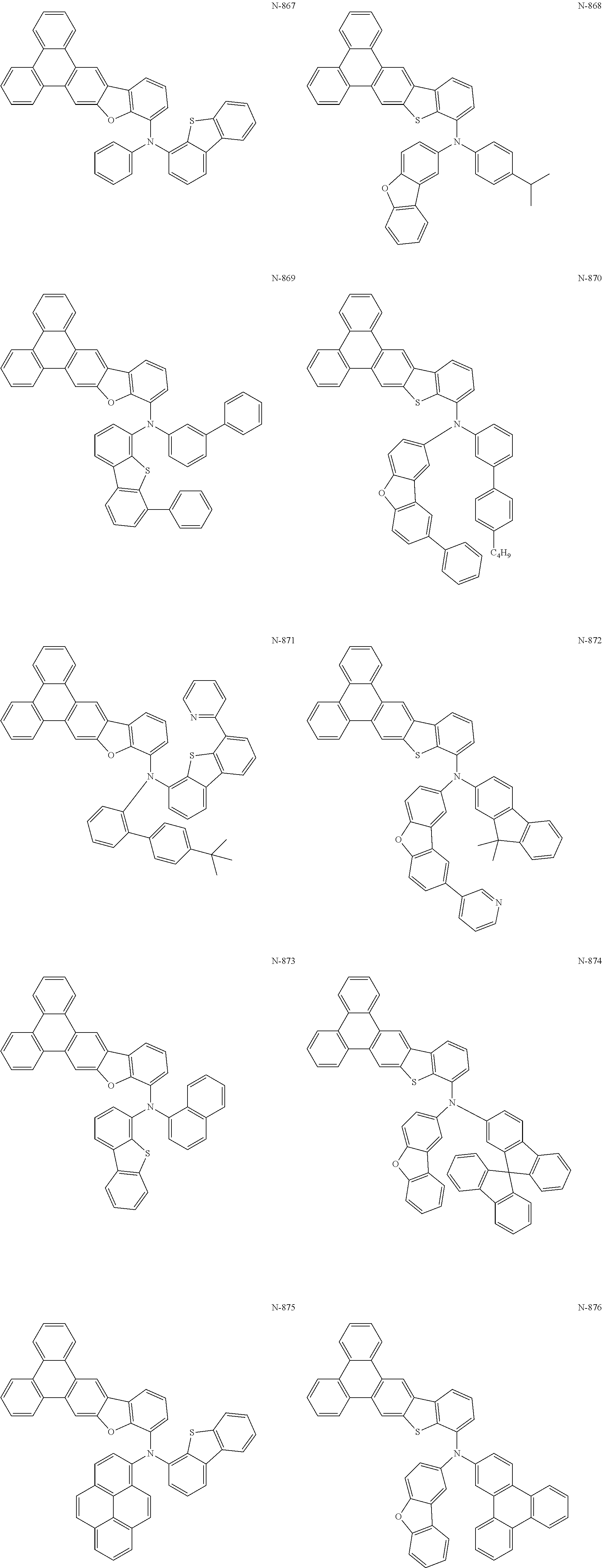

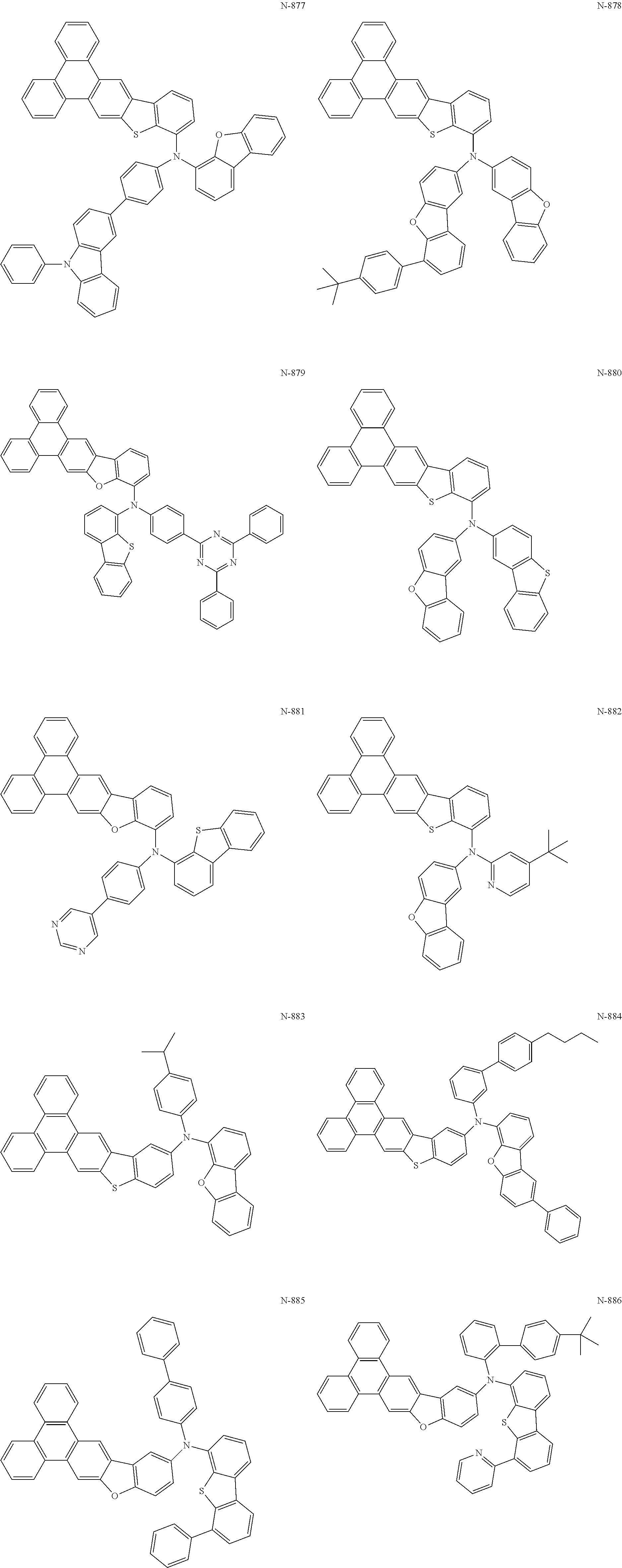

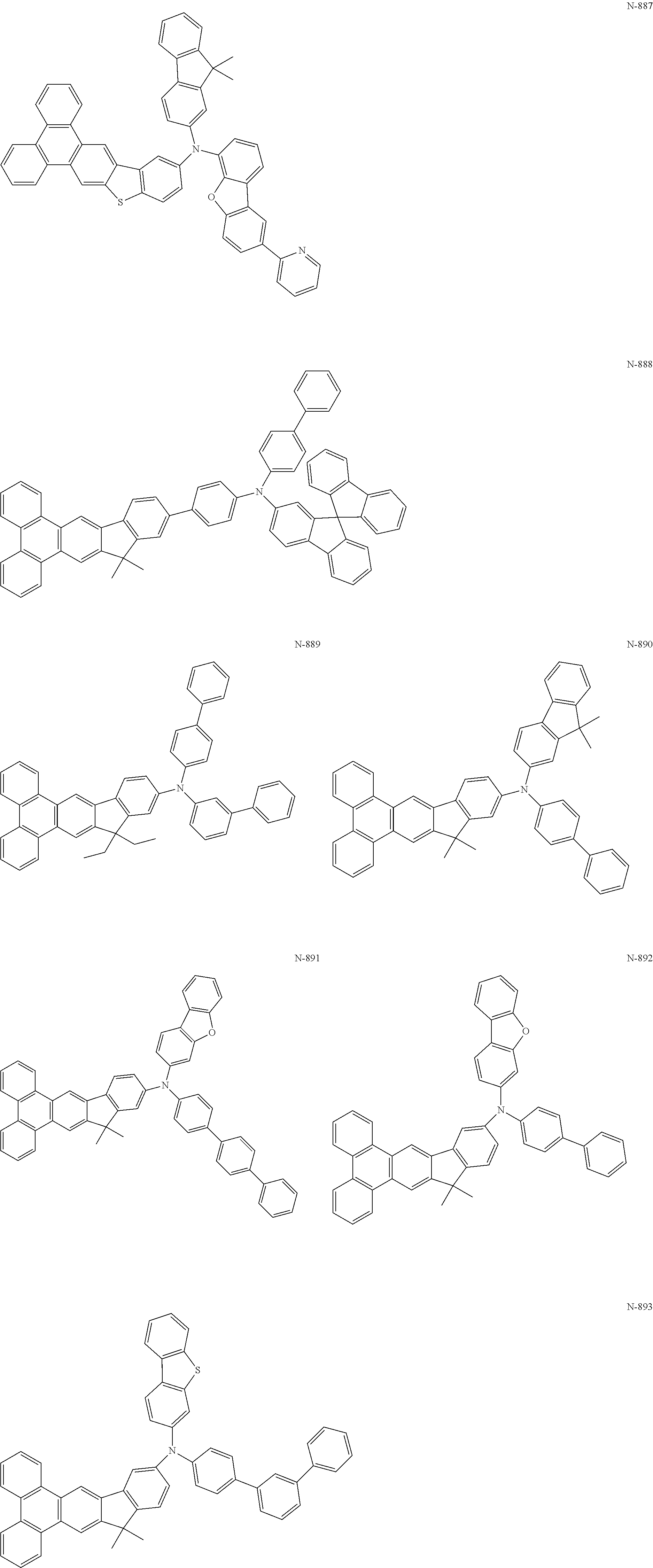

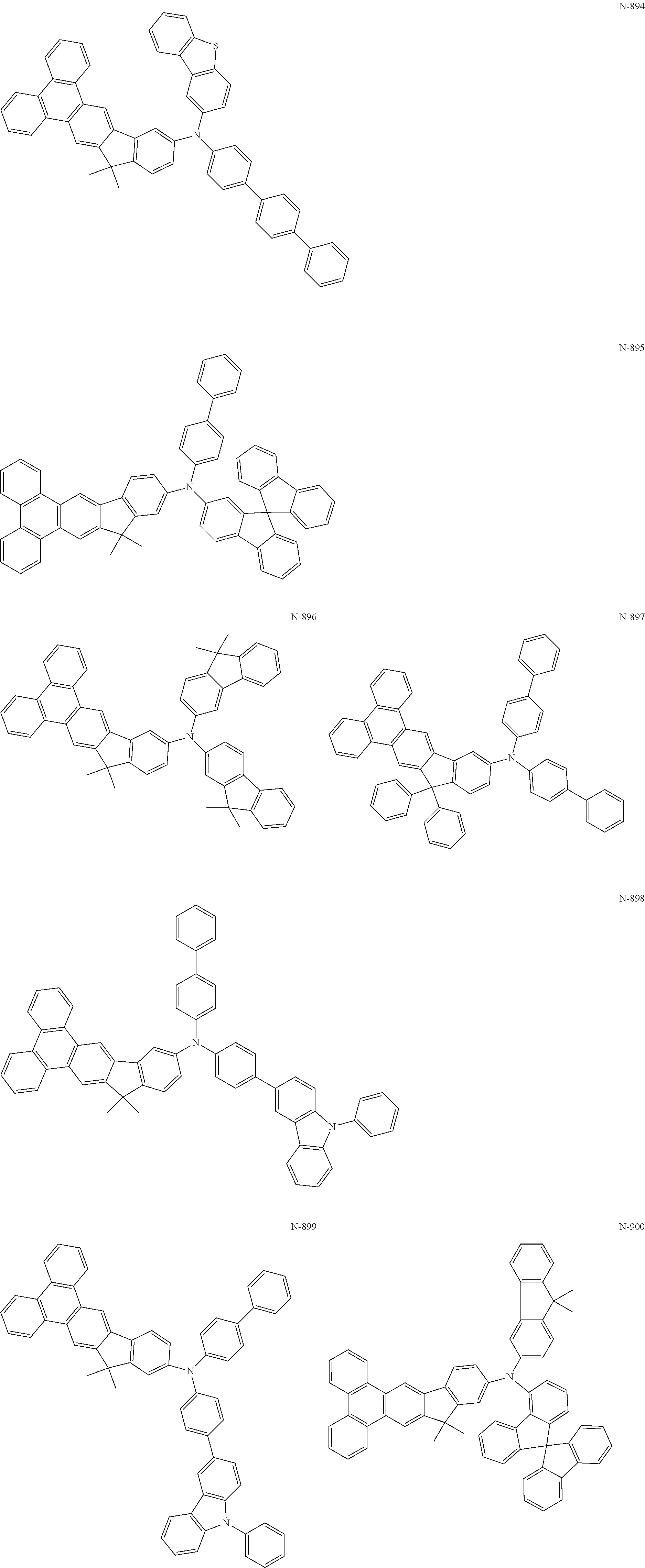

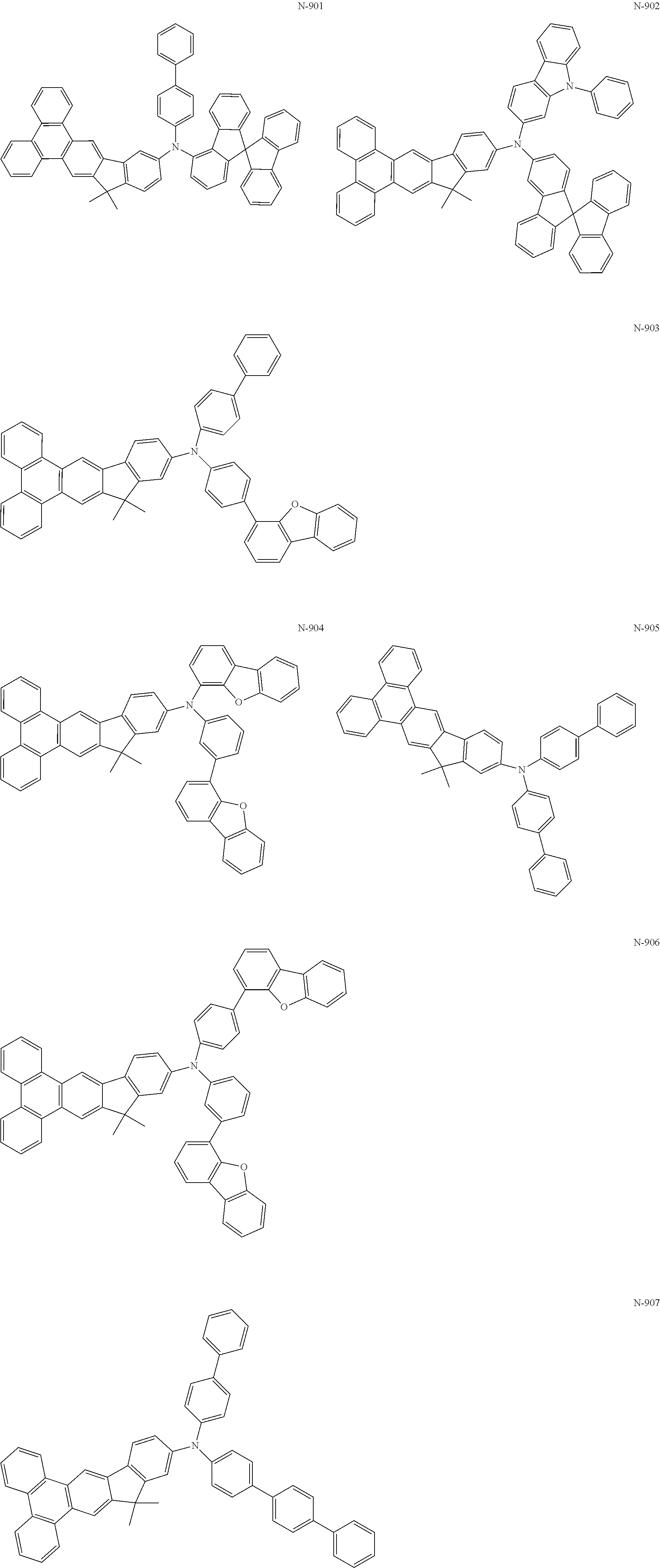

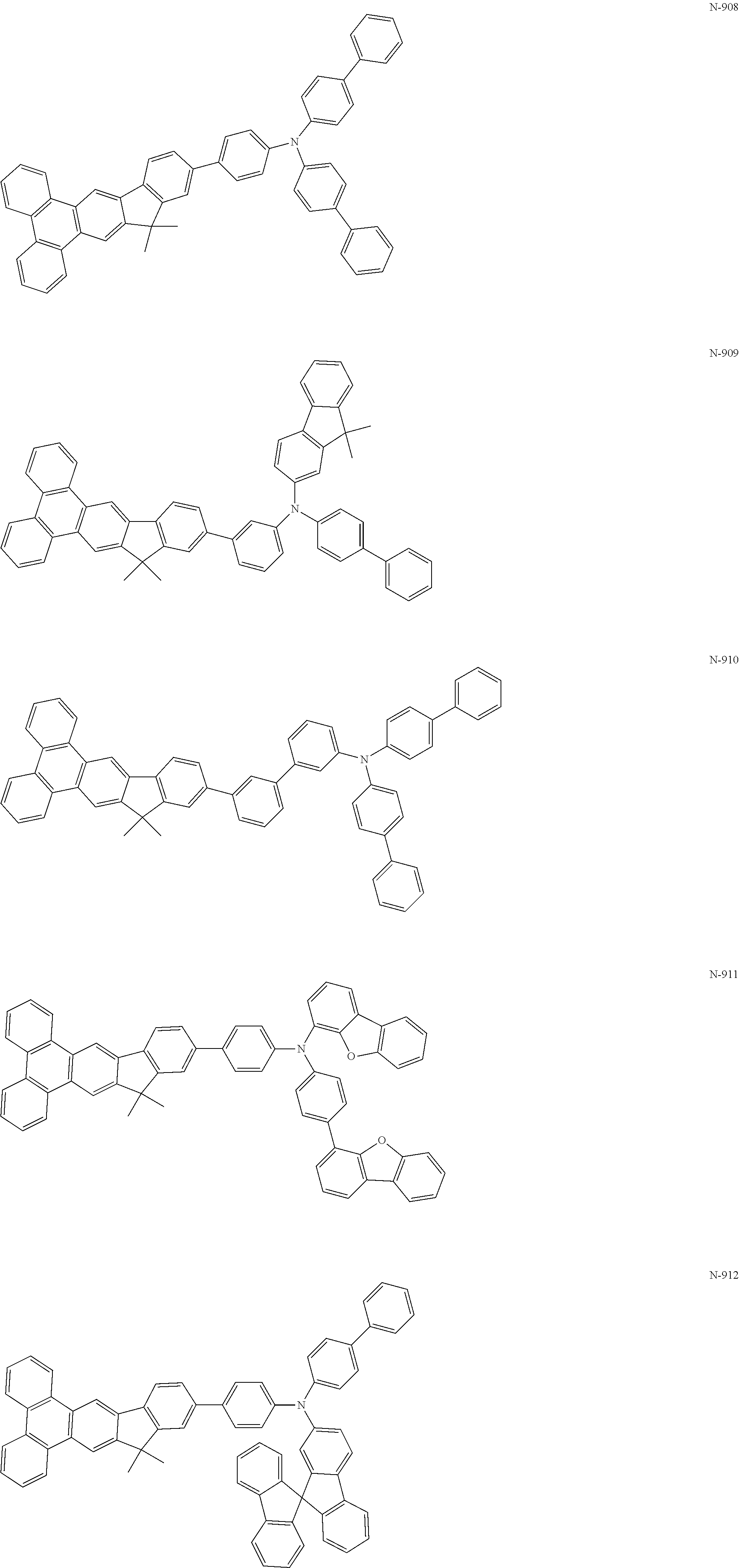

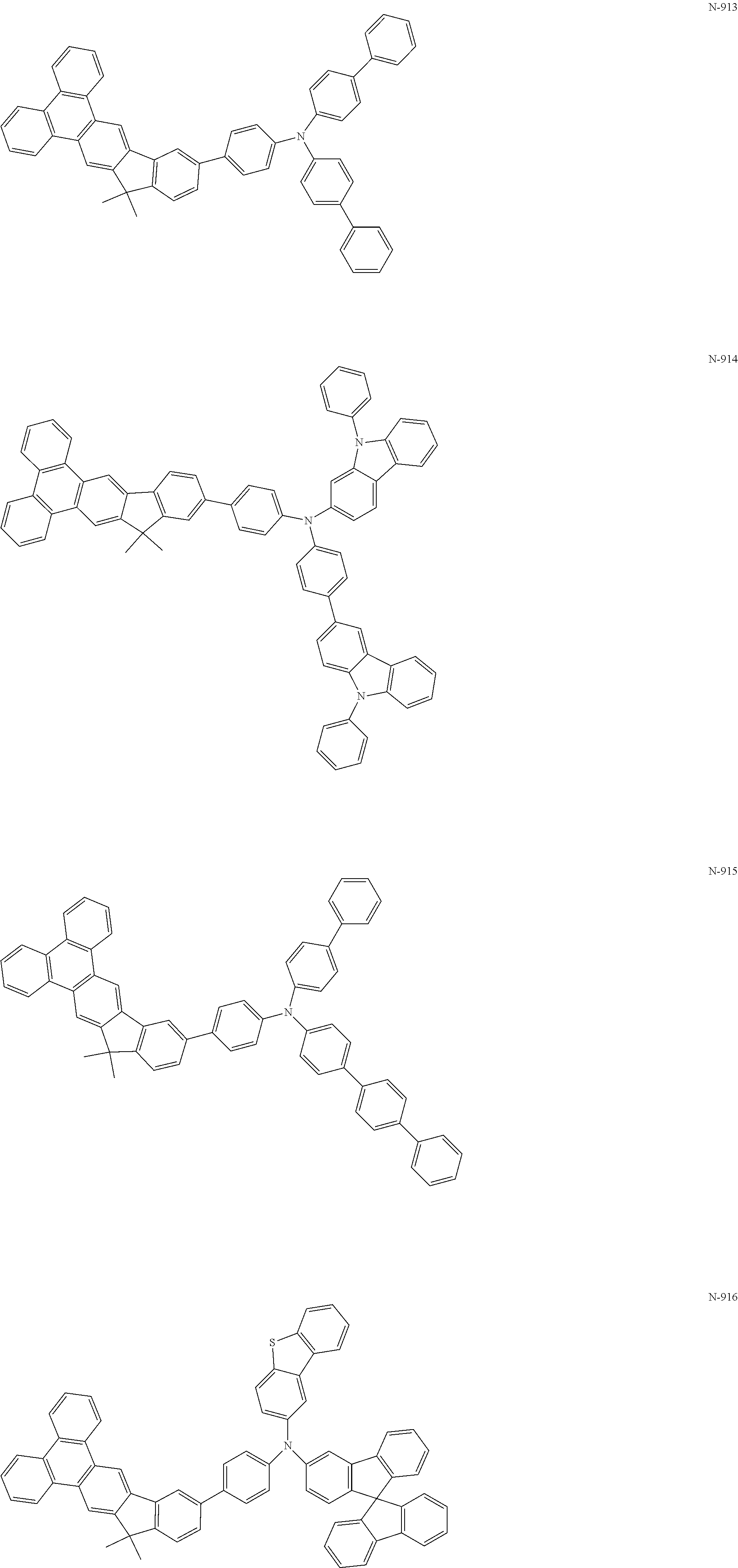 [SMILES: CC1(C)c2ccc(-c3ccc(N(c4ccc(-c5ccccc5)cc4)c4ccc(-c5ccccc5)cc4)cc3)cc2-c2cc3c4ccccc4c4ccccc4c3cc21 ; CC1(C)c2cc(-c3ccc(N(c4ccc(-c5ccc6c(c5)c5ccccc5n6-c5ccccc5)cc4)c4ccc5c6ccccc6n(-c6ccccc6)c5c4)cc3)ccc2-c2cc3c4ccccc4c4ccccc4c3cc21 ; CC1(C)c2ccc(-c3ccc(N(c4ccc(-c5ccccc5)cc4)c4ccc(-c5ccc(-c6ccccc6)cc5)cc4)cc3)cc2-c2cc3c4ccccc4c4ccccc4c3cc21 ; CC1(C)c2ccc(-c3ccc(N(c4ccc5c(c4)-c4ccccc4C54c5ccccc5-c5ccccc54)c4ccc5sc6ccccc6c5c4)cc3)cc2-c2cc3c4ccccc4c4ccccc4c3cc21]

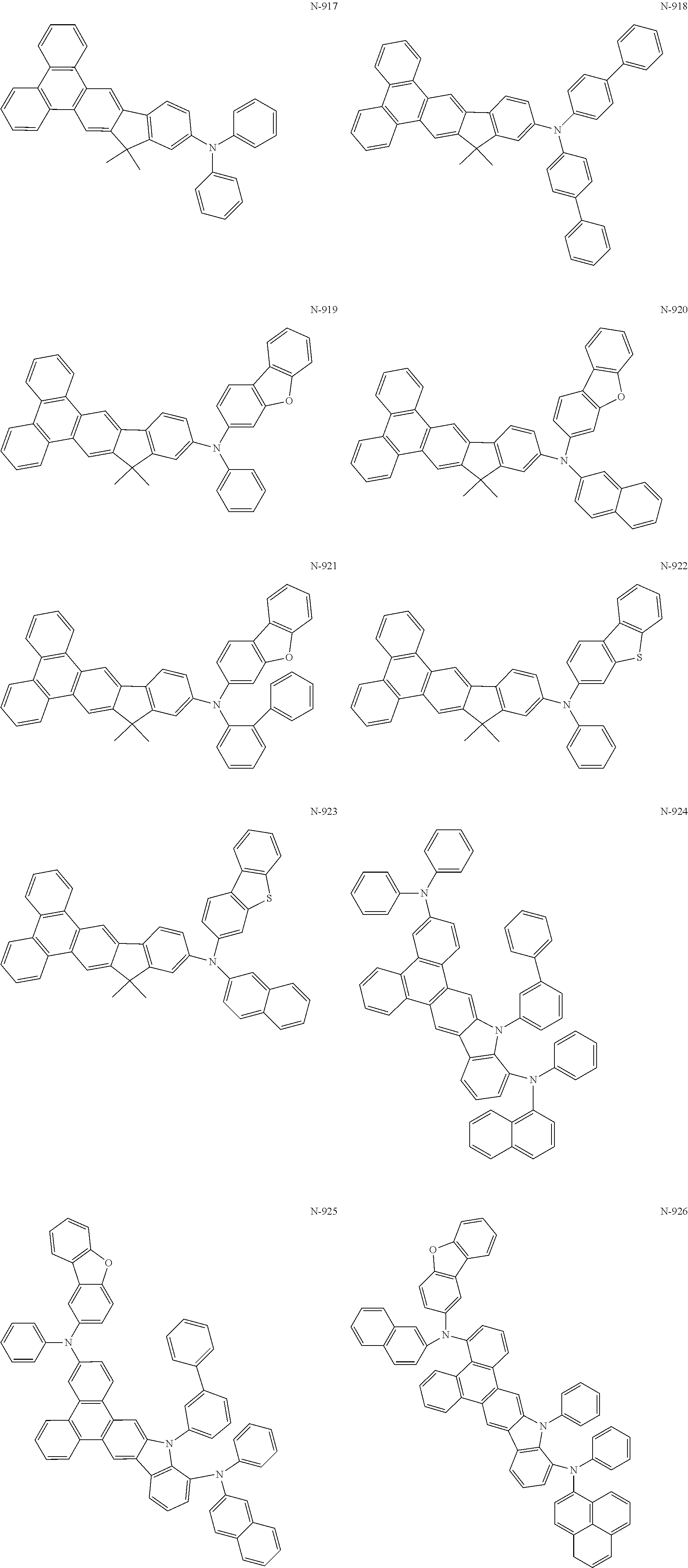

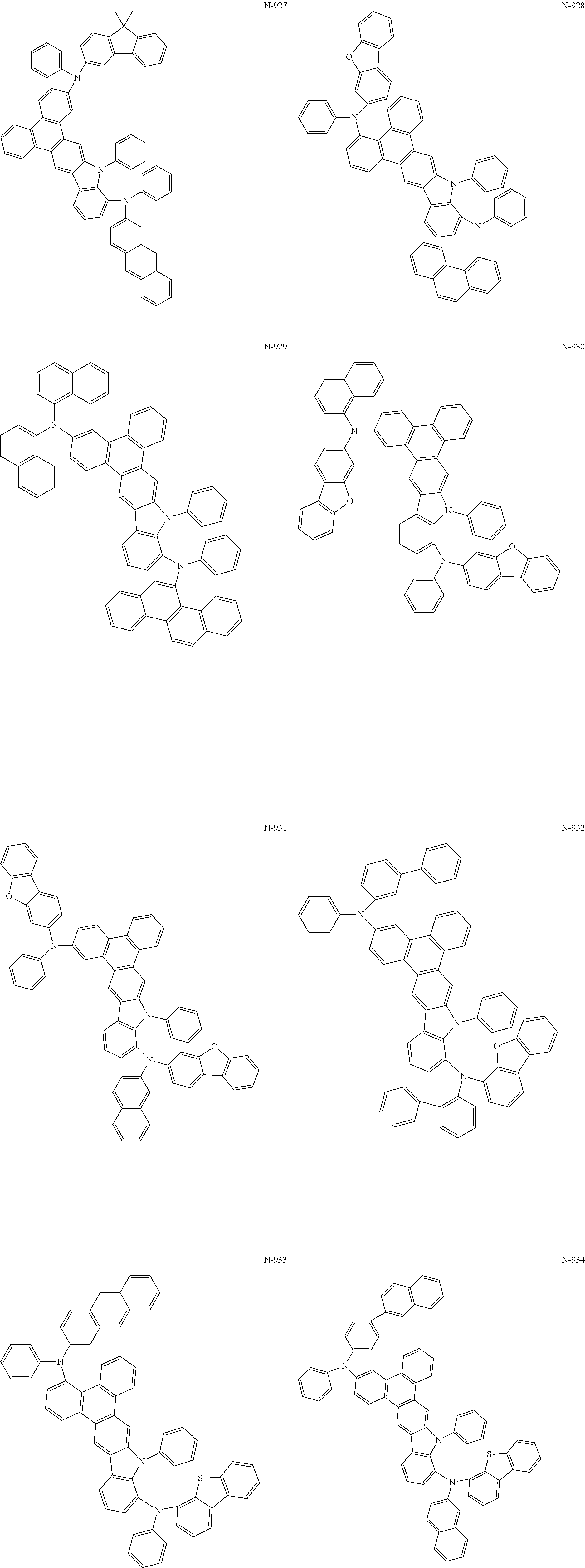

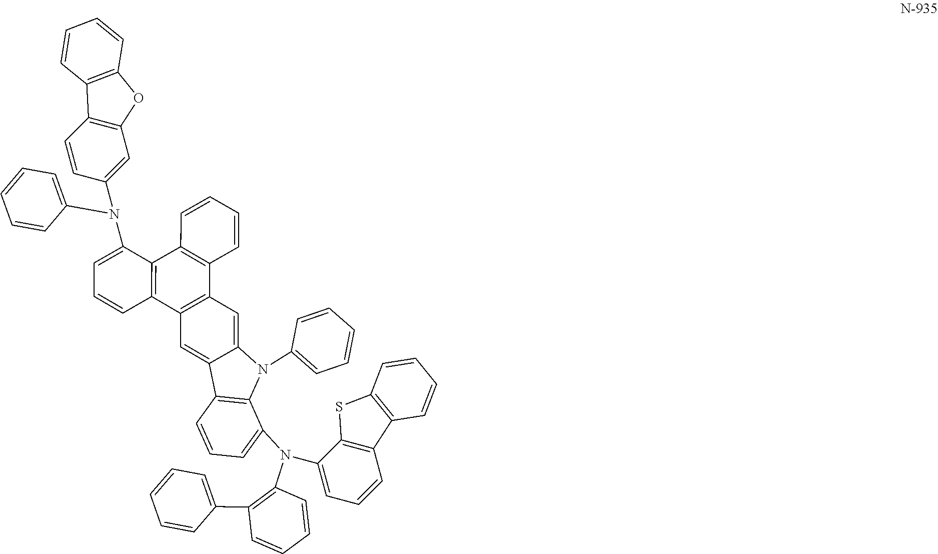 [SMILES: c1ccc(-c2ccccc2N(c2cccc3c2sc2ccccc23)c2cccc3c4cc5c(cc4n(-c4ccccc4)c23)c2ccccc2c2c(N(c3ccccc3)c3ccc4c(c3)oc3ccccc34)cccc52)cc1]

15. The composition according to claim 8, wherein a mass ratio of compound N to compound M is from 9:1 to 1:9; preferably from 2:8 to 8:2; and more preferably from 3:7 to 7:3, and further preferably from 4:6 to 6:4.

16. An application of the organic electroluminescent host material composition according to claim 8 in an optical device.

17. An organic electroluminescent material, comprising the organic electroluminescent compound according to claim 1.

18. An organic electroluminescent device, comprising an anode, a cathode and an organic layer arranged between the anode and the cathode; wherein the organic layer comprises the organic electroluminescent compound according to claim 1.

19. An organic electroluminescent equipment, comprising the organic electroluminescent device according to claim 18.

20. The application of the organic electroluminescent host material composition according to claim 16, wherein the optical device is an organic electroluminescent device.
